(19) United States
(12) Patent Application Publication
(10) Pub. No.: US 2025/0100996 A1
(43) Pub. Date: Mar. 27, 2025

(54) HETEROCYCLOALKYL AND HETEROARYL COMPOUNDS AND PHARMACEUTICAL COMPOSITIONS THAT MODULATE IKZF2

(71) Applicant: Plexium, Inc., San Diego, CA (US)

(72) Inventors: Pengyu YANG, San Diego, CA (US); Simon BAILEY, San Diego, CA (US)

(21) Appl. No.: 18/577,672

(22) PCT Filed: Jul. 8, 2022
(86) PCT No.: PCT/US2022/036520

§ 371 Date: Jan. 8, 2024

Related U.S. Application Data
(60) Provisional application No. 63/220,319, filed on Jul. 9, 2021.
(60) Provisional application No. 63/220,321, filed on Jul. 9, 2021.
(60) Provisional application No. 63/314,994, filed on Feb. 28, 2022.
(60) Provisional application No. 63/317,930, filed on Mar. 8, 2022.

(51) Int. Cl. C07D 401/14; A61K 31/454; A61K 31/4545; A61K 31/4709; A61K 31/497; A61K 31/498; A61K 31/501; A61K 31/506; A61K 31/5377; A61K 31/695; C07D 405/14; C07D 409/14; C07D 413/14; C07D 471/04; C07F 7/10

(52) U.S. Cl. CPC C07D 401/14; A61K 31/454; A61K 31/4545; A61K 31/4709; A61K 31/497; A61K 31/498; A61K 31/501; A61K 31/506; A61K 31/5377; A61K 31/695; C07D 405/14; C07D 409/14; C07D 413/14; C07D 471/04; C07F 7/10

(57) ABSTRACT

Disclosed are compounds I-A and I-B and salts thereof that bind to and modulate cereblon activity. In some embodiments, the binding and modulation of cereblon results in the degradation of IKAROS family zinc finger proteins (e.g., IKZF2).

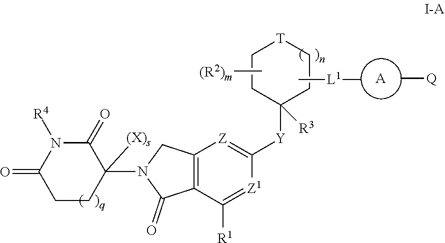

### CROSS-REFERENCE TO RELATED APPLICATIONS

This application claims the benefit under 35 U.S.C. § 119(e) of United States Provisional Application Nos. 63/220,319, filed Jul. 9, 2021, 63/220,321, filed Jul. 9, 2021, 63/314,994, filed Feb. 28, 2022, and 63/317,930, filed Mar. 8, 2022, which are hereby incorporated by reference in their entirety.

### FIELD

This disclosure provides for compounds and salts thereof that bind to cereblon, thereby modulating cereblon activity. In some embodiments, certain compounds described herein bind to cereblon, resulting in a reduction of cellular IKAROS Family Zinc Finger (IKZF) protein levels. In some embodiments, certain compounds described herein bind to cereblon, but do not result in a reduction of cellular IKZF protein levels. In some embodiments, certain compounds disclosed herein bind to cereblon, thereby initiating degradation of IKZF proteins (e.g., IKZF2). Also disclosed are pharmaceutical compositions comprising the compounds, or a salt (e.g., a pharmaceutically acceptable salt) thereof, and methods of using such compounds and/or their salts in the treatment of various IKZF2-mediated diseases or disorders.

### STATE OF THE ART

IKAROS Family Zinc Finger 2 (IKZF2) (also known as Helios) is one of the five members of the Ikaros family of transcription factors found in mammals. IKZF2 is a critical regulator of T cell activity and function. Genetic deletion of Helios resulted in an enhanced anti-tumor immune response (Kim et al., Science 350:334-339 (2015)). Notably, Helios is highly expressed in regulatory T cells (Tregs) (Elkord et al., Expert Opin. Biol. Ther. 12:1423-1425 (2012)), a subpopulation of T cells that restricts the activity of effector T cells. Selective deletion of Helios in regulatory T cells resulted in both loss of suppressive activity and acquisition of effector T cell functions (Najagawa et al., Proc. Natl. Acad. Sci. USA 113:6248-6253 (2016); Yates et al., Proc. Natl. Acad. Sci. USA 115:2162-2167 (2018)). Therefore, Helios is a critical factor in restricting T cell effector function in Tregs.

Helios expression has also been reported to be upregulated in “exhausted” T cells, in the settings of both chronic viral infections (Crawford et al., Immunity 40:289-302 (2014), Doering et al., Immunity 371130-1144 (2012); Scott-Browne et al., Immunity 45:1327-1340 (2016)) and tumors (Martinez et al., Immunity 42:265-278 (2015); Mognol et al., Proc. Natl. Acad. Sci. USA 114:E2776-E2785 (2017); Pereira et al., J. Leukoc. Biol. 102:601-615 (2017); Singer et al., Cell 166:1500-1511 (2016); Schietinger et al., Immunity 45:389-401 (2016)), as well as in dysfunctional chimeric antigen receptor (CAR) T cells (Long et al., Nat. Med. 21:581-590 (2015)) 16). Overexpression or aberrant expression of Helios and various splice isoforms have been reported in several hematological malignancies, including T cell leukemias and lymphomas (Nakase at al., Exp. Hematol. 30:313-317 (2002); Tabayashi et al., Cancer Sci. 98:182-188 (2007); Asanuma et al., Cancer Sci. 104:1097-1106 (2013)). Moreover, knockdown of Helios in a model of mixed lineage leukemia (MLL)-driven myeloid leukemia potently suppressed proliferation and increased cell death (Park et al., J. Clin. Invest. 125:1286-1298 (2015); Park et al., Cell Stem Cell 24:153-165 (2019)).

Currently, anti-CTLA4 antibodies are used in the clinic to target Tregs in tumors. However, targeting CTLA4 often causes systemic activation of T-effector cells, resulting in excessive toxicity and limiting therapeutic utility. Up to 75% of patients treated with a combination of anti-PD1 and anti-CTLA4 have reported grade 3 or higher adverse events (National Cancer Institute, Division of Cancer Treatment & diagnosis, Common Terminology for Adverse Events (CTCAE), https://ctep.cancer.gov/protocolDevelopment/electronic_applications/ctc.htm).

There is a need for therapies that can target Tregs in tumors without causing systemic activation of T-effector cells. Accordingly, an IKZF2-specific modulator or degrader would have the potential to focus an enhanced immune response to areas within or near tumors providing a potentially more tolerable and less toxic therapy for the treatment of diseases mediated by IKZF2.

### SUMMARY

Disclosed are compounds that bind to cereblon, thereby modulating cereblon activity. In some embodiments, certain compounds described herein bind to cereblon, resulting in a reduction of cellular IKAROS Family Zinc Finger (IKZF) protein levels. In some embodiments, certain compounds described herein bind to cereblon, but do not result in a reduction of cellular IKZF protein levels. In some embodiments, certain compounds disclosed herein bind to cereblon, thereby initiating degradation of IKZF proteins (e.g., IKZF2). Also disclosed are pharmaceutical compositions comprising the compounds, or a salt (e.g., a pharmaceutically acceptable salt) thereof, and methods of using such compounds and/or their salts in the treatment of various IKZF2-mediated diseases or disorders, including e.g., cancers.

In one embodiment, the disclosed compounds that bind to and modulate cereblon, and, in some instances, degrade IKZF2, are represented by formula I-A:

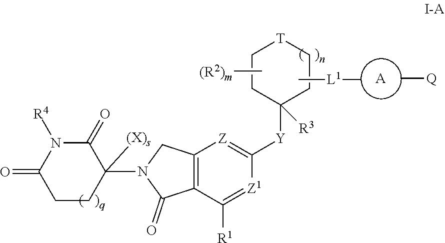

or a pharmaceutically acceptable salt, solvate, stereoisomer, or tautomer thereof, wherein:

m is zero, one, two, or three;

n is zero, one, or two;

q is one, two, or three;

s is one;

L¹ is CH₂ or a covalent bond;

Q is selected from —C(O)NR⁵R⁶ or a 4- to 10-member heterocyclic group having one or more heteroatoms independently selected from NR²⁰, O, S, and SO₂, where R²⁰ is —[C(O)]_{w}R⁷, S(O)₂R⁷, or a covalent bond linking said heterocyclic group to ring A, wherein said heterocyclic group is independently unsubstituted or substituted with C(O)R⁷, or with one to five R⁸, and where w is 0 or 1;

T is CH₂, CH(R²), C(R²)₂, or N, provided that when T is N, L¹ is attached to T;

X is hydrogen, deuterium, or fluoro;

Y is a covalent bond, —O—, or —NR—;

Z and Z¹ are each independently CR¹ or N;

ring A is phenyl or a 4- to 7-membered nitrogen containing heterocyclyl, wherein ring A is independently unsubstituted or substituted with one to five R⁸;

R is hydrogen or C₁-C₄ alkyl;

each R¹ is independently hydrogen, halo, cyano, hydroxy, —N(R⁹)₂, C₁-C₄ alkyl independently unsubstituted or substituted with from one to three R¹⁰, or C₁-C₆ alkoxy independently unsubstituted or substituted with from one to three R¹⁰; or

when Z¹ is CR¹, then two adjacent R¹ together with the carbon atoms to which they are attached form a C₃-C₇ cycloalkyl, a C₆-C₁₀ aryl, a 4- to 7-membered heterocycloalkenyl, or a 5- to 6-membered heteroaryl, wherein each of said cycloalkyl, heterocycloalkenyl, aryl, and heteroaryl are independently substituted with one to three R¹¹;

each R² is independently selected from halo, cyano, hydroxy, —N(R⁹)₂, C₁-C₄ alkyl independently unsubstituted or substituted with from one to three R¹⁰, and C₁-C₄ alkoxy independently unsubstituted or substituted with from one to three R¹⁰;

R³ is selected from the group consisting of hydrogen, amino, halo, cyano, hydroxy, C₁-C₆ alkyl, and C₁-C₆ haloalkyl;

R⁴ is hydrogen or —CH₂—OR¹²;

R⁵ and R⁶, together with the nitrogen to which they are attached, form a 4- to 7-membered heterocyclyl, wherein said heterocyclyl is independently unsubstituted or substituted with one to five R⁸;

R⁷ is hydrogen, C₁-C₆ alkyl, C₁-C₆ alkoxy, C₃-C₇ cycloalkyl, C₂-C₆ alkenyl, C₂-C₆ alkynyl, 4- to 7-membered heterocyclyl, C₆-C₁₀ aryl, 5- to 10-membered heteroaryl, or —N(R⁹)₂; wherein said alkyl, alkoxy, cycloalkyl, alkenyl, alkynyl, heterocyclyl, aryl, or heteroaryl is further independently unsubstituted or substituted with one to five R⁸;

each R⁸ is independently halo, cyano, hydroxy, —SH, —NH₂, —NO₂, —SF₅, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, heterocyclyl, aryl, or heteroaryl;

each R⁹ is independently H or C₁-C₄ alkyl independently unsubstituted or substituted with from 1 to 3 substituents selected from halo, cyano, hydroxy, and C₁-C₄ alkoxy, or two R⁹ together with the nitrogen atom to which they are attached form a 3- to 7-membered heterocycloalkyl or 5- to 6-membered heteroaryl;

each R¹⁰ is independently —N(R⁹)₂, halo, cyano, hydroxy, or C₁-C₄ alkoxy;

each R¹¹ is independently —N(R⁹)₂, halo, cyano, hydroxy, or oxo;

R¹² is —C(O)—R¹³ or —P(O)(OR¹⁴)₂;

R¹³ is C₁-C₄ alkyl or C₁-C₄ alkoxy; and

each R⁴ is independently H or C₁-C₄ alkyl.

In one embodiment, the disclosed compounds that bind to and modulate cereblon, and, in some instances, degrade IKZF2, are represented by formula I-A′:

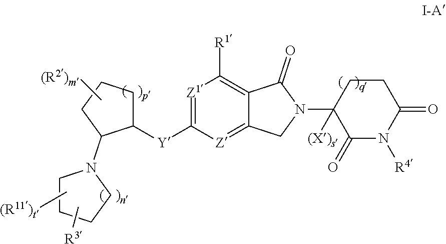

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, n′, p′, q′, s′, and t′ of formula I-A′ are as defined in the detailed description and throughout the specification.

In one embodiment, the disclosed compounds that bind to and modulate cereblon, and, in some instances, degrade IKZF2, are represented by formula II-A′:

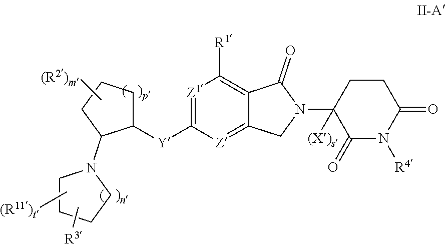

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, n′, p′, s′, and t′ of formula II-A′ are as defined in the detailed description and throughout the specification.

In one embodiment, the disclosed compounds that bind to and modulate cereblon, and, in some instances, degrade IKZF2, are represented by formula III-A′:

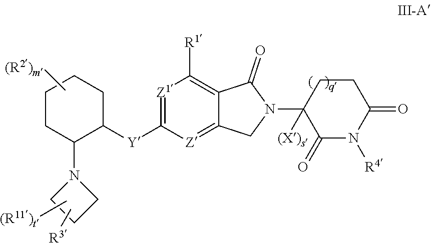

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, q′, s′, and t′ of formula III-A′ are as defined in the detailed description and throughout the specification.

In one embodiment, the disclosed compounds that bind to and modulate cereblon, and, in some instances, degrade IKZF2, are represented by formula IV-A′:

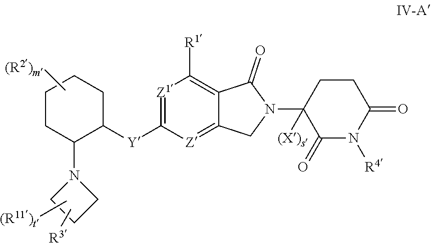

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, s′, and t′ of formula IV-A′ are as defined in the detailed description and throughout the specification.

In some embodiments, the disclosed compounds that bind to and modulate cereblon, and, in some instances, degrade IKZF2, are represented by formula V-A′:

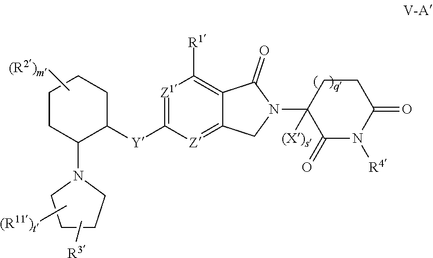

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, q′, s′, and t′ of formula V-A′ are as defined in the detailed description and throughout the specification.

In some embodiments, the disclosed compounds that bind to and modulate cereblon, and, in some instances, degrade IKZF2, are represented by formula VI-A′:

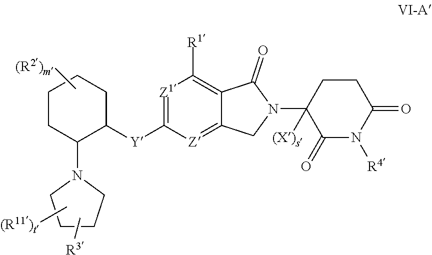

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, s′, and t′ of formula VI-A′ are as defined in the detailed description and throughout the specification.

In one embodiment, the disclosed compounds that bind to and modulate cereblon, and, in some instances, degrade IKZF2, are represented by formula I-B:

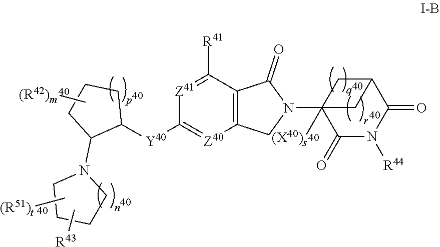

or a pharmaceutically acceptable salt, solvate, stereoisomer, and/or tautomer thereof, wherein R⁴¹, R⁴², R⁴³, R⁴⁴, R⁵¹, X⁴⁰, Y⁴⁰, Z⁴⁰, Z⁴¹, M⁴⁰, n⁴⁰, p⁴⁰, q⁴⁰, r⁴⁰, s⁴⁰, and t⁴⁰ of formula I-B are as defined in the detailed description and throughout the specification.

In one embodiment, the disclosed compounds that bind to and modulate cereblon, and, in some instances, degrade IKZF2, are represented by formula II-B:

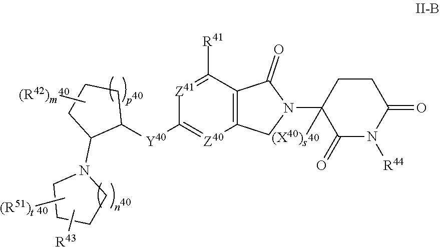

or a pharmaceutically acceptable salt, solvate, stereoisomer, and/or tautomer thereof, wherein R⁴¹, R⁴², R⁴³, R⁴⁴, R⁵¹, X⁴⁰, Y⁴⁰, Z⁴⁰, Z⁴¹, m⁴⁰, n⁴⁰, p⁴⁰, s⁴⁰, and t⁴⁰ of formula II-B are as defined in the detailed description and throughout the specification.

In one embodiment, the disclosed compounds that bind to and modulate cereblon, and, in some instances, degrade IKZF2, are represented by formula III-B:

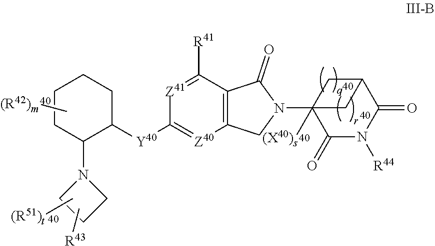

or a pharmaceutically acceptable salt, solvate, stereoisomer, and/or tautomer thereof, wherein R⁴¹, R⁴², R⁴³, R⁴⁴, R⁵¹, X⁴⁰, Y⁴⁰, Z⁴⁰, Z⁴¹, m⁴⁰, q⁴⁰, r⁴⁰, s⁴⁰, and t⁴⁰ of formula III-B are as defined in the detailed description and throughout the specification.

In one embodiment, the disclosed compounds that bind to and modulate cereblon, and, in some instances, degrade IKZF2, are represented by formula IV-B:

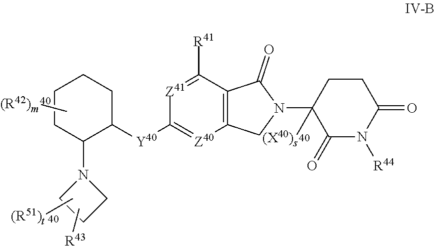

or a pharmaceutically acceptable salt, solvate, stereoisomer, and/or tautomer thereof, wherein R⁴¹, R⁴², R⁴³, R⁴⁴, R⁵¹, X⁴⁰, Y⁴⁰, Z⁴⁰, Z⁴¹, m⁴⁰, s⁴⁰, and t⁴⁰ of formula IV-B are as defined in the detailed description and throughout the specification.

In some embodiments, the disclosed compounds that bind to and modulate cereblon, and, in some instances, degrade IKZF2, are represented by formula V-B:

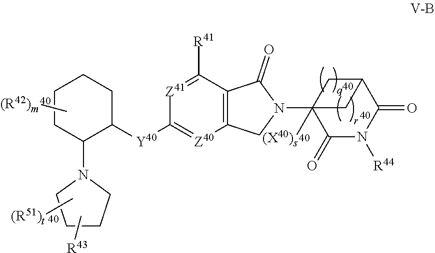

or a pharmaceutically acceptable salt, solvate, stereoisomer, and/or tautomer thereof, wherein R⁴¹, R⁴², R⁴³, R⁴⁴, R⁵¹, X⁴⁰, Y⁴⁰, Z⁴⁰, Z⁴¹, m⁴⁰, q⁴⁰, r⁴⁰, s⁴⁰, and t⁴⁰ of formula V-B are as defined in the detailed description and throughout the specification.

In some embodiments, the disclosed compounds that bind to and modulate cereblon, and, in some instances, degrade IKZF2, are represented by formula VI-B:

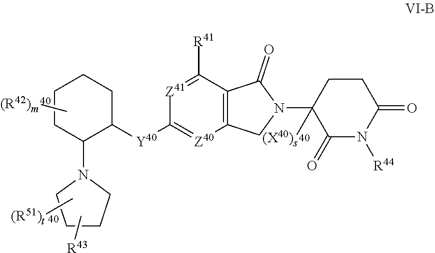

or a pharmaceutically acceptable salt, solvate, stereoisomer, and/or tautomer thereof, wherein R⁴¹, R⁴², R⁴³, R⁴⁴, R⁵¹, X⁴⁰, Y⁴⁰, Z⁴⁰, Z⁴¹, m⁴⁰, s⁴⁰, and t⁴⁰ of formula VI-B are as defined in the detailed description and throughout the specification.

In one embodiment, provided is a compound of formula I-A, formula I-A′, or formula I-B, or a sub-formulae of a compound of formula I-A, formula I-A′, or formula I-B, which selectively modulates IKZF (e.g., over translation termination factor GSPT1 (GI to S phase transition 1 protein)). In one embodiment, provided is a compound of formula I-A, formula I-A′, or formula I-B, or a sub-formulae of a compound of formula I-A, formula I-A′, or formula I-B, which selectively modulates IKZF2 over GSPT1.

In one embodiment, provided is a composition comprising a compound of formula I-A or any sub-formulae thereof, or a pharmaceutically acceptable salt, solvate, stereoisomer, or tautomer of a compound of formula I-A or any sub-formulae thereof. “Compound of formula I-A and sub-formulae thereof” refers to compounds of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, or IXB-A as defined in the detailed description and throughout the specification.

In one embodiment, provided is a composition comprising a compound of formula I-A′ or any sub-formulae thereof, or a pharmaceutically acceptable salt, solvate, stereoisomer, or tautomer of a compound of formula I-A′ or any sub-formulae thereof. “Compound of formula I-A′ and sub-formulae thereof” refers to compounds of formula I-A′, II-A′, III-A′, IV-A′, V-A′, and VI-A′ as defined in the detailed description and throughout the specification.

In one embodiment, provided is a composition comprising a compound of formula I-B or any sub-formulae thereof or a pharmaceutically acceptable salt, solvate, stereoisomer, or tautomer of a compound of formula I-B or any sub-formulae thereof. “Compound of formula I-B and sub-formulae thereof” refers to compounds of formula I-B, II-B, III-B, IV-B, V-B, and VI-B as defined in the detailed description and throughout the specification.

In one embodiment, this disclosure provides for a method for modulating cereblon, which method comprises contacting cereblon with an effective amount of a compound of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, or VI-B, or a pharmaceutically acceptable salt, solvate, stereoisomer, or tautomer thereof under conditions wherein cereblon is modulated.

In one embodiment, this disclosure provides for a method for degrading IKZF2, which method comprises contacting IKZF2 with an effective amount of a compound of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, or VI-B, or a pharmaceutically acceptable salt, solvate, stereoisomer, or tautomer thereof under conditions wherein IKZF2 is degraded.

In one embodiment, there is provided a method to degrade IKZF2 in a subject, which method comprises administering to said subject an effective amount of a compound of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, or VI-B, or a pharmaceutically acceptable salt, solvate, stereoisomer, or tautomer thereof, or administering to said subject a pharmaceutical composition comprising a pharmaceutically acceptable excipient and an effective amount of a compound of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, or VI-B, or a pharmaceutically acceptable salt, solvate, stereoisomer, or tautomer thereof.

Further provided is a method to treat cancer in a subject in need thereof, which method comprises selecting a subject whose cancer is mediated at least in part by IKZF2 and administering to said subject an effective amount of a compound of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, or VI-B, or a pharmaceutically acceptable salt, solvate, stereoisomer, or tautomer thereof, or administering to said subject a pharmaceutical composition comprising a pharmaceutically acceptable excipient and an effective amount of a compound of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, or VI-B, or a pharmaceutically acceptable salt, solvate, stereoisomer, or tautomer thereof.

### DETAILED DESCRIPTION

This disclosure provides for compounds, pharmaceutical compositions comprising such compounds, and methods of using such compounds and compositions to treat diseases, disorders, or conditions mediated, at least in part, by IKZF2 transcription factors. However, prior to providing a detailed description of the disclosure, the following terms will first be defined. If not defined, terms used herein have their generally accepted scientific meaning.

### Definitions

The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

The term “about” when used before a numerical designation, e.g., temperature, time, amount, concentration, and such other, including a range, indicates approximations which may vary by (+) or (−) 10%, 5%, 1%, or any subrange or subvalue there between. In one embodiment, the term “about” when used with regard to a dose amount means that the dose may vary by +/−10%.

“Comprising” or “comprises” is intended to mean that the compositions and methods include the recited elements, but not excluding others.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure.

“Consisting of” shall mean excluding more than trace elements of other ingredients and substantial method steps. Embodiments defined by each of these transition terms are within the scope of this disclosure.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” group (for example, methylenyl, ethylenyl, and propylenyl), an “arylene” group or an “arylenyl” group (for example, phenylenyl or napthylenyl, or quinolinyl for heteroarylene), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of acyl include, e.g., formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein, or R^{y} and R^{z} are taken together to form a cycloalkyl or heterocyclyl; each of which may be unsubstituted or substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be unsubstituted or substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be unsubstituted or substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₄ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be unsubstituted or substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be unsubstituted or substituted, as defined herein.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl, or heteroaryl; each of which may be unsubstituted or substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be unsubstituted or substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl”—used interchangeably with “heterocycloalkyl”—refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system. In some embodiments, the heterocycloalkyl may be substituted with oxo group(s) on a heteroatom (e.g., S═O, S(═O)₂).

“Oxime” refers to the group —CR^{y}(═NOH) wherein R^{y} is hydrogen, alkyl, alkenyl, alkynyl, cycloalkyl, heterocyclyl, aryl, heteroalkyl, or heteroaryl; each of which may be unsubstituted or substituted, as defined herein.

“Oxo” refers to the moiety ═O.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be unsubstituted or substituted, as defined herein. Examples of sulfinyl are methylsulfinyl, ethylsulfinyl, phenylsulfinyl, and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl, or heteroaryl; each of which may be unsubstituted or substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, =NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide, or —Si(R^{y})₃, wherein each R^{y} is independently hydrogen, alkyl, alkenyl, alkynyl, heteroalkyl, cycloalkyl, aryl, heteroaryl, or heterocyclyl.

In certain embodiments, “substituted” includes any of the above alkyl, alkenyl, alkynyl, cycloalkyl, heterocyclyl, aryl, or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(O)R^{h}, —NR^{g}C(O)NR^{g}R^{h}, —NR^{g}C(O)OR^{h}, —NR^{g}S(O)₁₋₂R^{h}, —C(O)R^{g}, —C(O)OR^{g}, —OC(O)OR^{g}, —OC(O)R^{g}, —C(O)NR^{g}R^{h}, —OC(O)NR^{g}R^{h}, —OR^{g}, —SR^{g}, —S(O)R^{g}, —S(O)₂R^{g}, —OS(O)₁₋₂R^{g}, —S(O)₁₋₂OR^{g}, —NR^{g}S(O)₁₋₂NR^{g}R^{h}, ═NSO₂R^{g}, =NOR^{g}, —S(O)₁₋₂NR^{g}R^{h}, —SF₅, —SCF₃, or —OCF₃. In certain embodiments, “substituted” also means any of the above groups in which one or more (e.g., 1 to 5 or 1 to 3) hydrogen atoms are replaced with —C(O)R^{g}, —C(O)OR^{g}, —C(O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxy, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} are taken together with the atoms to which they are attached to form a heterocyclyl ring, unsubstituted or substituted with oxo, halo, or alkyl unsubstituted or substituted with oxo, halo, amino, hydroxy, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number.

Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to absorption, distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, or ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Provided are also or a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### Compounds

In one embodiment, the disclosed compounds that bind to and modulate cereblon, and, in some instances, degrade IKZF2, are represented by formula I-A:

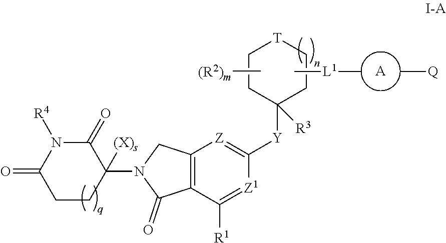

or a pharmaceutically acceptable salt, solvate, stereoisomer, or tautomer thereof, wherein:

m is zero, one, two, or three;

n is zero, one, or two;

q is one, two, or three;

s is one;

L¹ is CH₂ or a covalent bond;

Q is selected from —C(O)NR⁵R⁶ or a 4- to 10-member heterocyclic group having one or more heteroatoms independently selected from NR²⁰, O, S, and SO₂, where R²⁰ is —[C(O)]_{w}R⁷, S(O)₂R⁷, or a covalent bond linking said heterocyclic group to ring A, wherein said heterocyclic group is independently unsubstituted or substituted with C(O)R⁷, or with one to five R⁸, and where w is 0 or 1;

T is CH₂, CH(R²), C(R²)₂, or N, provided that when T is N, L¹ is attached to T;

X is hydrogen, deuterium, or fluoro;

Y is a covalent bond, —O—, or —NR—;

Z and Z¹ are each independently CR¹ or N;

ring A is phenyl or a 4- to 7-membered nitrogen containing heterocyclyl, wherein ring A is independently unsubstituted or substituted with one to five R⁸;

R is hydrogen or C₁-C₄ alkyl;

each R¹ is independently hydrogen, halo, cyano, hydroxy, —N(R⁹)₂, C₁-C₄ alkyl independently unsubstituted or substituted with from one to three R¹⁰, or C₁-C₆ alkoxy independently unsubstituted or substituted with from one to three R¹⁰; or

when Z¹ is CR¹, then two adjacent R¹ together with the carbon atoms to which they are attached form a C₃-C₇ cycloalkyl, a C₆-C₁₀ aryl, a 4- to 7-membered heterocycloalkenyl, or a 5- to 6-membered heteroaryl, wherein each of said cycloalkyl, heterocycloalkenyl, aryl, and heteroaryl are independently substituted with one to three R¹¹;

each R² is independently selected from halo, cyano, hydroxy, —N(R⁹)₂, C₁-C₄ alkyl independently unsubstituted or substituted with from one to three R¹⁰, and C₁-C₄ alkoxy independently unsubstituted or substituted with from one to three R¹⁰;

R³ is selected from the group consisting of hydrogen, amino, halo, cyano, hydroxy, C₁-C₆ alkyl, and C₁-C₆ haloalkyl;

R⁴ is hydrogen or —CH₂—OR¹²;

R⁵ and R⁶, together with the nitrogen to which they are attached, form a 4- to 7-membered heterocyclyl, wherein said heterocyclyl is independently unsubstituted or substituted with one to five R⁸;

R⁷ is hydrogen, C₁-C₆ alkyl, C₁-C₆ alkoxy, C₃-C₇ cycloalkyl, C₂-C₆ alkenyl, C₂-C₆ alkynyl, 4- to 7-membered heterocyclyl, C₆-C₁₀ aryl, 5- to 10-membered heteroaryl, or —N(R⁹)₂; wherein said alkyl, alkoxy, cycloalkyl, alkenyl, alkynyl, heterocyclyl, aryl, or heteroaryl is further independently unsubstituted or substituted with one to five R⁸;

each R⁸ is independently halo, cyano, hydroxy, —SH, —NH₂, —NO₂, —SF₅, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, heterocyclyl, aryl, or heteroaryl;

each R⁹ is independently H or C₁-C₄ alkyl independently unsubstituted or substituted with from 1 to 3 substituents selected from halo, cyano, hydroxy, and C₁-C₄ alkoxy, or two R⁹ together with the nitrogen atom to which they are attached form a 3- to 7-membered heterocycloalkyl or 5- to 6-membered heteroaryl;

each R¹⁰ is independently —N(R⁹)₂, halo, cyano, hydroxy, or C₁-C₄ alkoxy;

each R¹¹ is independently —N(R⁹)₂, halo, cyano, hydroxy, or oxo;

R¹² is —C(O)—R¹³ or —P(O)(OR¹⁴)₂;

R¹³ is C₁-C₄ alkyl or C₁-C₄ alkoxy; and

each R¹⁴ is independently H or C₁-C₄ alkyl.

In some embodiments, Q is

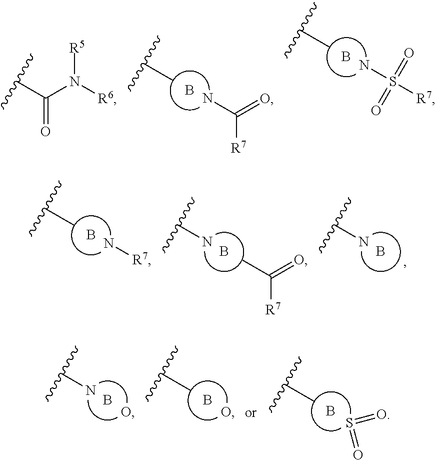

In some embodiments of compounds of formula I-A, T is N and L¹ is CH₂. In some embodiments, ring A is phenyl or piperidinyl. In some embodiments, ring A is phenyl and Q is

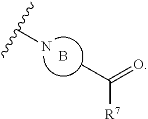

In some embodiments, ring B is defined as in Q above. In some embodiments, ring B is a 4- to 7-membered heterocyclyl containing one or more heteroatoms selected from nitrogen, oxygen, and sulfur, or a combination thereof, wherein ring B is independently unsubstituted or substituted with one to five R⁸.

In some embodiments of compounds of formula I-A, R⁷ is aryl, C₁-C₆ alkyl substituted with C₃-C₇ cycloalkyl, C₁-C₆ alkoxy, 4- to 7-membered heterocyclyl substituted with C₁-C₆ alkyl, or C₃-C₇ cycloalkyl independently unsubstituted or substituted with one or two C₁-C₆ alkyl, halo, or cyano.

It is to be understood that the independent substitution of the R⁷ moiety with C₁-C₆ alkyl, or C₃-C₇ cycloalkyl independently unsubstituted or substituted with one or two C₁-C₆ alkyl, halo, or cyano, may occur at any location in the R⁷ moiety with an appropriate valence for said substitution.

In some embodiments of compounds of formula I-A, T is CH₂, n is 1, and m is zero. In some embodiments of compounds of formula I-A, T is C(R²)₂, n is 1, m is zero, and R² is fluoro. In some embodiments of compounds of formula I-A, T is CH₂, n is 1, m is two, and R² is fluoro. In some embodiments of compounds of formula I-A,

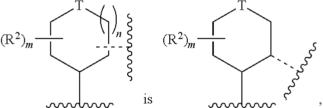

wherein the dotted exocyclic bond is attached to L¹, and the solid exocyclic bond is attached to Y. In some embodiments of compounds of formula I-A,

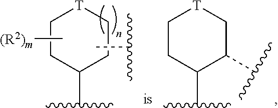

wherein the dotted exocyclic bond is attached to L¹, and the solid exocyclic bond is attached to Y. In some embodiments of compounds of formula I-A,

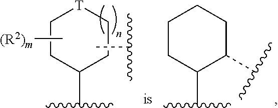

wherein the dotted exocyclic bond is attached to L¹, and the solid exocyclic bond is attached to Y. In some embodiments of compounds of formula I-A,

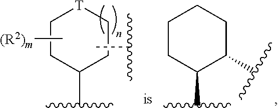

wherein the dotted exocyclic bond is attached to L¹, and the solid exocyclic bond is attached to Y. In some embodiments of compounds of formula I-A,

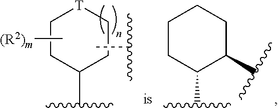

wherein the dotted exocyclic bond is attached to L¹, and the solid exocyclic bond is attached to Y. In some embodiments of compounds of formula I-A,

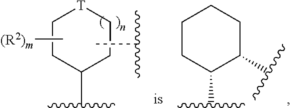

wherein the dotted exocyclic bond is attached to L¹, and the solid exocyclic bond is attached to Y. In some embodiments of compounds of formula I-A,

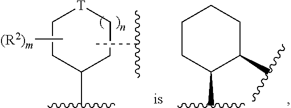

wherein the dotted exocyclic bond is attached to L¹, and the solid exocyclic bond is attached to Y. In some embodiments of compounds of formula I-A,

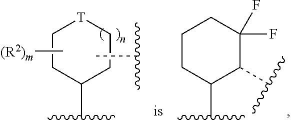

wherein the dotted exocyclic bond is attached to L¹, and the solid exocyclic bond is attached to Y.

In some embodiments, the compound of formula I-A, that binds to and modulates cereblon, and, in some instances, degrades IKZF2, is represented by formula II-A:

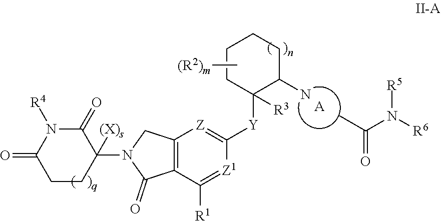

or a pharmaceutically acceptable salt, solvate, stereoisomer, or tautomer thereof, wherein ring A, R¹, R², R³, R⁴, R⁵, R⁶, X, Y, Z, Z¹, m, n, q, and s are as defined herein, and wherein ring A is independently unsubstituted or substituted with one to five R⁸.

It is to be understood that the independent substitution of ring A with one to five R⁸ substituents may occur at any location in ring A with an appropriate valence for said substitution.

In some embodiments, for any compound of formula II-A or sub-formulae thereof, or a pharmaceutically acceptable salt, solvate, stereoisomer, and/or tautomer thereof,

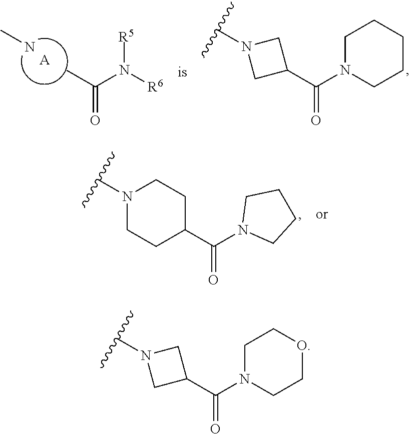

In some embodiments, in a compound of formula I-A or formula II-A, or a pharmaceutically acceptable salt, solvate, stereoisomer, or tautomer thereof, X is hydrogen or deuterium. In some embodiments, X is hydrogen. In some embodiments, X is deuterium.

In some embodiments, in a compound of formula I-A or formula II-A, or a pharmaceutically acceptable salt, solvate, stereoisomer, or tautomer thereof, X is fluoro.

In some embodiments, in a compound of formula I-A or formula II-A, or a pharmaceutically acceptable salt, solvate, stereoisomer, or tautomer thereof, n is 0. In some embodiments, in a compound of formula I-A or formula II-A, or a pharmaceutically acceptable salt, solvate, stereoisomer, or tautomer thereof, n is 1. In some embodiments, n is 2. In some embodiments, n is 3.

In some embodiments, in a compound of formula I-A or formula II-A, or a pharmaceutically acceptable salt, solvate, stereoisomer, or tautomer thereof, R⁴ is hydrogen.

In some embodiments, in a compound of formula I-A or formula II-A, or a pharmaceutically acceptable salt, solvate, stereoisomer, and/or tautomer thereof, Z and Z¹ are each C—R¹. In some embodiments, Z and Z¹ are each CH. In some embodiments, Z and Z¹ are each N. In some embodiments, one of Z or Z¹ is C—R¹ and the other of Z or Z¹ is N. In some embodiments, Z and Z¹ are each C—R¹, wherein one R¹ is hydrogen, and the other R¹ is C₁-C₆ alkoxy. In some embodiments, Z and Z¹ are each C—R¹, wherein one R¹ is hydrogen, and the other R¹ is a halogen, such as bromo, chloro, or fluoro. In some embodiments, one of Z or Z¹ is C—H and the other of Z or Z¹ is N. In some embodiments, R¹ is hydrogen. In some embodiments, Z and Z¹ are each CH and R¹ is hydrogen.

In some embodiments, in a compound of formula I-A or formula II-A, or a pharmaceutically acceptable salt, solvate, stereoisomer, or tautomer thereof, m is zero. In some embodiments, m is 1. In some embodiments, m is 2.

In some embodiments, in a compound of formula I-A or formula II-A, or a pharmaceutically acceptable salt, solvate, stereoisomer, or tautomer thereof, q is 1. In some embodiments, in a compound of formula I-A or formula II-A, or a pharmaceutically acceptable salt, solvate, stereoisomer, or tautomer thereof, q is 2. In some embodiments, in a compound of formula I-A or formula II-A, or a pharmaceutically acceptable salt, solvate, stereoisomer, or tautomer thereof, q is 3.

In some embodiments, the compound of formula II-A, that binds to and modulates cereblon, and, in some instances, degrades IKZF2, is represented by formula IIA-A:

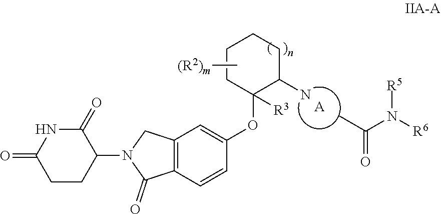

or a pharmaceutically acceptable salt, solvate, stereoisomer, or tautomer thereof, wherein ring A, R², R³, R⁵, R⁶, m, and n are as defined herein, and wherein ring A is independently unsubstituted or substituted with one to five R⁸.

It is to be understood that the independent substitution of ring A with one to five R¹ substituents may occur at any location in ring A with an appropriate valence for said substitution.

In some embodiments, the compound of formula IIA-A, that binds to and modulates cereblon, and, in some instances, degrades IKZF2, is represented by formula IIB-A:

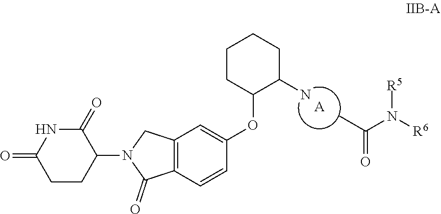

or a pharmaceutically acceptable salt, solvate, stereoisomer, or tautomer thereof, wherein ring A, R⁵, and R⁶ are as defined herein, and wherein ring A is independently unsubstituted or substituted with one to five R⁸.

It is to be understood that the independent substitution of ring A with one to five R⁸ substituents may occur at any location in ring A with an appropriate valence for said substitution.

In some embodiments, the compound of formula IIB-A, that binds to and modulates cereblon, and, in some instances, degrades IKZF2, is represented by formula IIC-A:

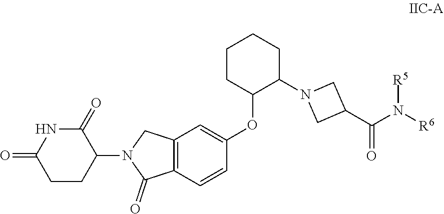

or a pharmaceutically acceptable salt, solvate, stereoisomer, or tautomer thereof, wherein R⁵ and R⁶ are as defined herein.

In some embodiments, this disclosure provides a compound of formula I-A, II-A, IIA-A, IIB-A, or IIC-A, or a pharmaceutically acceptable salt, solvate, stereoisomer, or tautomer thereof, wherein R⁵ and R⁶, together with the nitrogen to which they are attached, form a 4- to 7-membered heterocyclyl group optionally having an additional 1 to 2 heteroatoms selected from N, NR, O, and S, wherein said heterocyclyl is independently unsubstituted or substituted with one to five R⁸. It is to be understood that the independent substitution of the 4- to 7-membered heterocyclyl group with one to five R¹ substituents may occur at any location in the 4- to 7-membered heterocyclyl group with an appropriate valence for said substitution. In some embodiments, NR⁵R⁶ is

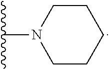 [SMILES: *N1CCCCC1]

In some embodiments, the compound of formula I-A, that binds to and modulates cereblon, and, in some instances, degrades IKZF2, is represented by formula III-A:

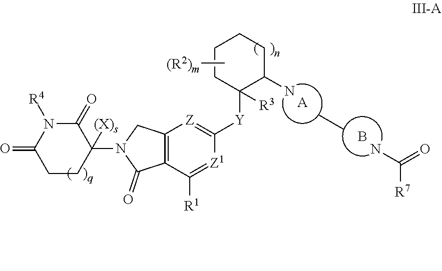

or a pharmaceutically acceptable salt, solvate, stereoisomer, or tautomer thereof, wherein ring A, ring B, R¹, R², R³, R⁴, R⁷, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

It is to be understood that the ring B nitrogen bearing the acyl group can be located at any position in ring B.

In some embodiments, for any compound of formula III-A or sub-formulae thereof, or a pharmaceutically acceptable salt, solvate, stereoisomer, and/or tautomer thereof, R⁷ is C₁-C₆ alkyl. In some embodiments, for any compound of formula III-A or sub-formulae thereof, or a pharmaceutically acceptable salt, solvate, stereoisomer, and/or tautomer thereof, R⁷ is an independently unsubstituted or substituted C₃-C₇ cycloalkyl. In some embodiments, for any compound of formula III-A or sub-formulae thereof, or a pharmaceutically acceptable salt, solvate, stereoisomer, and/or tautomer thereof, R⁷ is an independently unsubstituted or substituted 4- to 7-membered heterocyclyl. In some embodiments, for any compound of formula III-A or sub-formulae thereof, or a pharmaceutically acceptable salt, solvate, stereoisomer, and/or tautomer thereof, R⁷ is an independently unsubstituted or substituted C₆-C₁₀ aryl. In some embodiments, for any compound of formula III-A or sub-formulae thereof, or a pharmaceutically acceptable salt, solvate, stereoisomer, and/or tautomer thereof, R⁷ is an independently unsubstituted or substituted C₁-C₆ alkyl. In some embodiments, for any compound of formula III-A or sub-formulae thereof, or a pharmaceutically acceptable salt, solvate, stereoisomer, and/or tautomer thereof,

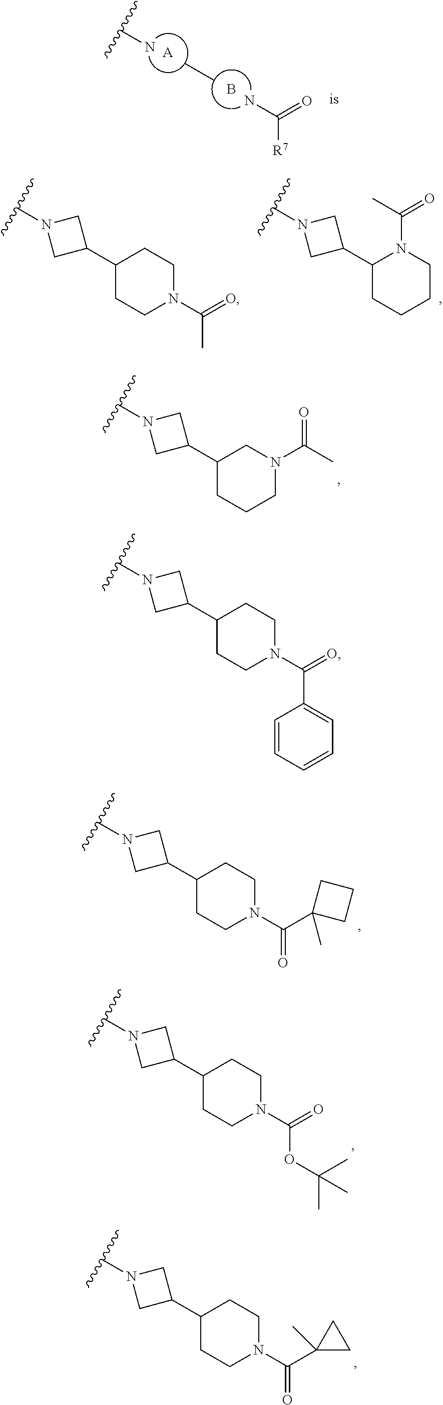

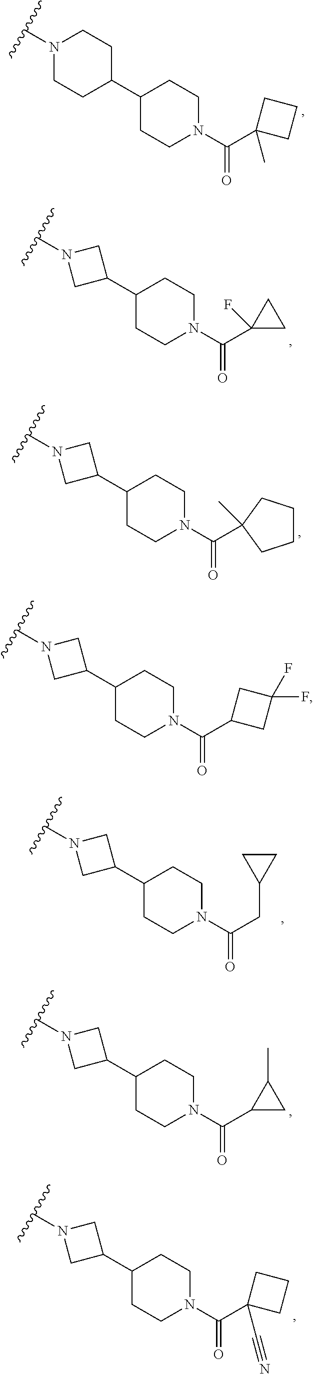

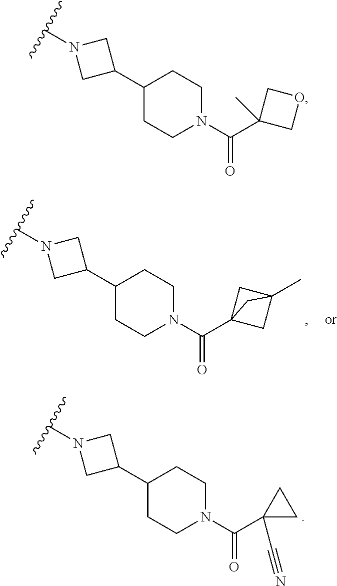 [SMILES: *N1CC(C2CCN(C(=O)C3(C)COC3)CC2)C1 ; *N1CC(C2CCN(C(=O)C34CC(C)(C3)C4)CC2)C1 ; *N1CC(C2CCN(C(=O)C3(C#N)CC3)CC2)C1]

In some embodiments, the compound of formula III-A, that binds to and modulates cereblon, and, in some instances, degrades IKZF2, is represented by formula IIIA-A:

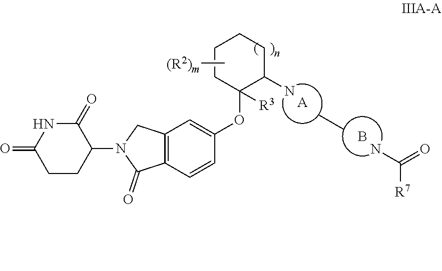

or a pharmaceutically acceptable salt, solvate, stereoisomer, or tautomer thereof, wherein ring A, ring B, R², R³, R⁷, m, and n are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula IIIA-A, that binds to and modulates cereblon, and, in some instances, degrades IKZF2, is represented by formula IIIB-A:

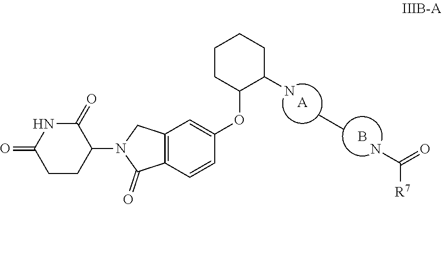

or a pharmaceutically acceptable salt, solvate, stereoisomer, or tautomer thereof, wherein ring A, ring B, and R⁷ are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula IIIB-A, that binds to and modulates cereblon, and, in some instances, degrades IKZF2, is represented by formula IIIC-A:

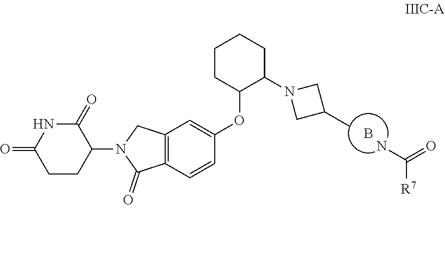

or a pharmaceutically acceptable salt, solvate, stereoisomer, or tautomer thereof, wherein ring B and R⁷ are as defined herein, and wherein ring B is independently unsubstituted or substituted with one to five R⁸.

It is to be understood that the independent substitution of ring B with one to five R⁸ substituents may occur at any location in ring B with an appropriate valence for said substitution.

In some embodiments, the compound of formula IIIC-A, that binds to and modulates cereblon, and, in some instances, degrades IKZF2, is represented by formula IIID-A:

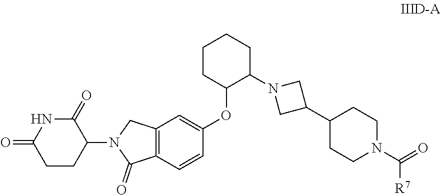

or a pharmaceutically acceptable salt, solvate, stereoisomer, or tautomer thereof, wherein R⁷ is as defined herein.

In some embodiments, this disclosure provides a compound of formula III-A, IIIA-A, IIIB-A, IIIC-A, or IIID-A, or a pharmaceutically acceptable salt, solvate, stereoisomer, or tautomer thereof, wherein R⁷ is C₁-C₆ alkyl, C₁-C₆ alkoxy, C₃-C₇ cycloalkyl, 4- to 7-membered heterocyclyl, or C₆-C₁₀ aryl, wherein said alkyl, cycloalkyl, heterocyclyl, or aryl is further independently unsubstituted or substituted with 1 to 3 substituents selected from halo, cyano, C₁-C₄ alkyl, and C₃-C₇ cycloalkyl. In some embodiments, R⁷ is C₃-C₇ cycloalkyl or 4- to 7-membered heterocyclyl independently unsubstituted or substituted with 1 to 3 substituents selected from halo, cyano, C₁-C₄ alkyl, and C₃-C₇ cycloalkyl.

In some embodiments, the compound of formula I-A, that binds to and modulates cereblon, and, in some instances, degrades IKZF2, is represented by formula IV-A:

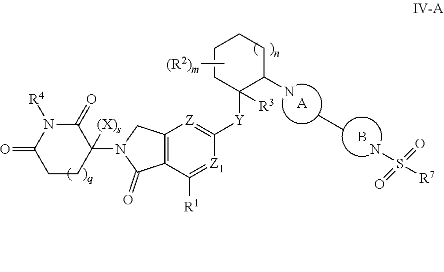

or a pharmaceutically acceptable salt, solvate, stereoisomer, or tautomer thereof, wherein ring A, ring B, R¹, R², R³, R⁴, R⁷, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments of compounds of formula IV-A,

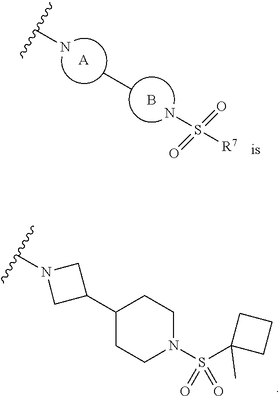

In some embodiments of compounds of formula IV-A,

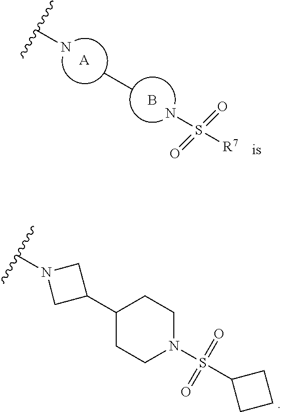

In some embodiments, the compound of formula IV-A, that binds to and modulates cereblon, and, in some instances, degrades IKZF2, is represented by formula IVA-A:

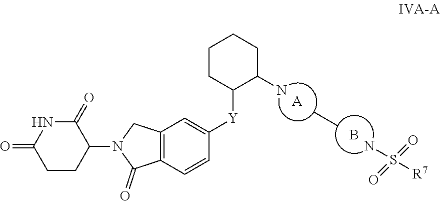

or a pharmaceutically acceptable salt, solvate, stereoisomer, or tautomer thereof, wherein Y, ring A, ring B, and R⁷ are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula IVA-A, that binds to and modulates cereblon, and, in some instances, degrades IKZF2, is represented by formula IVB-A:

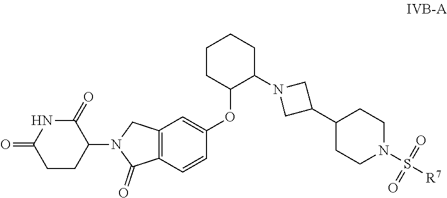

or a pharmaceutically acceptable salt, solvate, stereoisomer, or tautomer thereof, wherein R⁷ is as defined herein.

In some embodiments, this disclosure provides a compound of formula IV-A, IVA-A, or IVB-A, or a pharmaceutically acceptable salt, solvate, stereoisomer, or tautomer thereof, wherein R⁷ is C₃-C₇ cycloalkyl independently unsubstituted or substituted with 1 to 3 substituents selected from halo, cyano, C₁-C₄ alkyl, and C₃-C₇ cycloalkyl.

In some embodiments, the compound of formula I-A, that binds to and modulates cereblon, and, in some instances, degrades IKZF2, is represented by formula V-A:

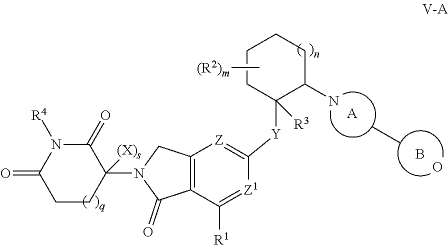

or a pharmaceutically acceptable salt, solvate, stereoisomer, or tautomer thereof, wherein ring A, ring B, R¹, R², R³, R⁴, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, for any compound of formula V-A or sub-formulae thereof, or a pharmaceutically acceptable salt, solvate, stereoisomer, and/or tautomer thereof, R¹ is hydroxy. In some embodiments, for any compound of formula V-A or sub-formulae thereof, or a pharmaceutically acceptable salt, solvate, stereoisomer, and/or tautomer thereof, R⁸ is halo, such as chloro, fluoro, or bromo.

In some embodiments of compounds of formula V-A, the heterocyclyl ring B is a heterocycloalkyl group. In some embodiments of compounds of formula V-A, the heterocyclyl ring B is a heterocycloalkenyl group.

In some embodiments of compounds of formula V-A,

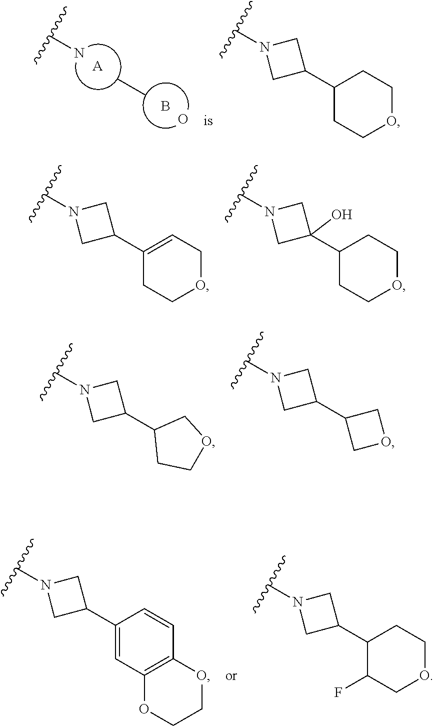

In some embodiments, the compound of formula V-A, that binds to and modulates cereblon, and, in some instances, degrades IKZF2, is represented by formula VA-A:

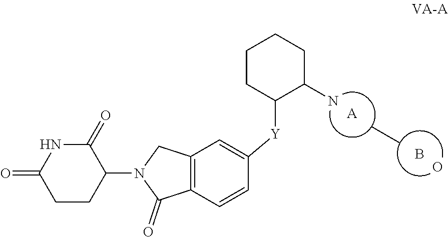

or a pharmaceutically acceptable salt, solvate, stereoisomer, or tautomer thereof, wherein Y, ring A, and ring B are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula V-A, that binds to and modulates cereblon, and, in some instances, degrades IKZF2, is represented by formula VB-A:

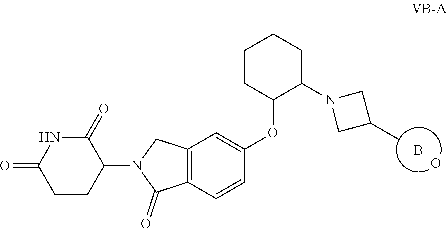

or a pharmaceutically acceptable salt, solvate, stereoisomer, or tautomer thereof, wherein ring B is as defined herein, and wherein ring B is independently unsubstituted or substituted with one to five R⁸.

It is to be understood that the independent substitution of ring B with one to five R¹ substituents may occur at any location in ring B with an appropriate valence for said substitution.

In some embodiments, this disclosure provides a compound of formula V-A, VA-A, or VB-A, or a pharmaceutically acceptable salt, solvate, stereoisomer, or tautomer thereof, wherein R¹ is hydroxy.

In some embodiments, the compound of formula I-A, that binds to and modulates cereblon, and, in some instances, degrades IKZF2, is represented by formula VI-A:

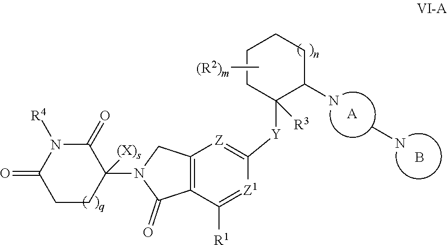

or a pharmaceutically acceptable salt, solvate, stereoisomer, or tautomer thereof, wherein ring A, ring B, R¹, R², R³, R⁴, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, for any compound of formula VI-A or sub-formulae thereof, or a pharmaceutically acceptable salt, solvate, stereoisomer, and/or tautomer thereof, R¹ is C₁-C₆ alkyl. In some embodiments, for any compound of formula VI-A or sub-formulae thereof, or a pharmaceutically acceptable salt, solvate, stereoisomer, and/or tautomer thereof, R⁸ is halo. In some embodiments of compounds of formula VI-A,

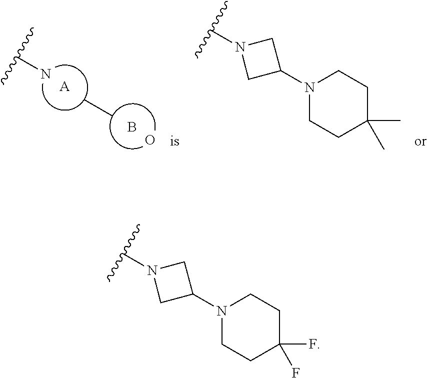

In some embodiments, the compound of formula VI-A, that binds to and modulates cereblon, and, in some instances, degrades IKZF2, is represented by formula VIA-A:

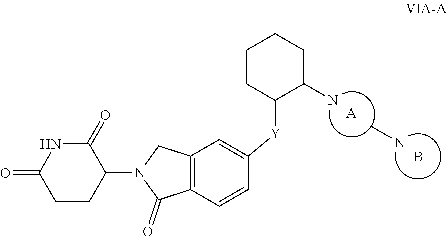

or a pharmaceutically acceptable salt, solvate, stereoisomer, or tautomer thereof, wherein Y, ring A, and ring B are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula VI-A, that binds to and modulates cereblon, and, in some instances, degrades IKZF2, is represented by formula VIB-A:

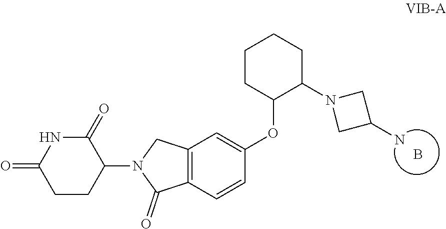

or a pharmaceutically acceptable salt, solvate, stereoisomer, or tautomer thereof, wherein ring B is as defined herein, and wherein ring B is independently unsubstituted or substituted with one to five R⁸.

It is to be understood that the independent substitution of ring B with one to five R⁸ substituents may occur at any location in ring B with an appropriate valence for said substitution.

In some embodiments, this disclosure provides a compound of formula VI-A, VIA-A, or VIB-A, or a pharmaceutically acceptable salt, solvate, stereoisomer, or tautomer thereof, wherein R⁸ is C₁-C₆ alkyl or halo.

In some embodiments, the compound of formula I-A, that binds to and modulates cereblon, and, in some instances, degrades IKZF2, is represented by formula VII-A:

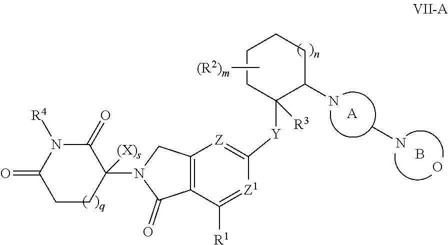

or a pharmaceutically acceptable salt, solvate, stereoisomer, or tautomer thereof, wherein ring A, ring B, R¹, R², R³, R⁴, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments of compounds of formula VII-A,

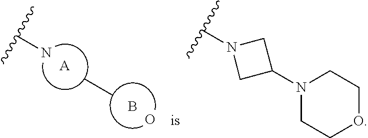

In some embodiments, the compound of formula VII-A, that binds to and modulates cereblon, and, in some instances, degrades IKZF2, is represented by formula VIIA-A:

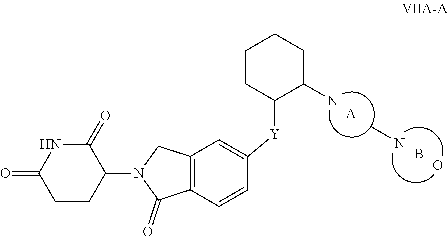

or a pharmaceutically acceptable salt, solvate, stereoisomer, or tautomer thereof, wherein Y, ring A, and ring B are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula VII-A, that binds to and modulates cereblon, and, in some instances, degrades IKZF2, is represented by formula VIIB-A:

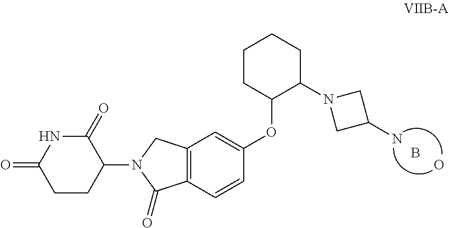

or a pharmaceutically acceptable salt, solvate, stereoisomer, or tautomer thereof, wherein ring B is as defined herein, and wherein ring B is independently unsubstituted or substituted with one to five R⁸.

It is to be understood that the independent substitution of ring B with one to five R⁸ substituents may occur at any location in ring B with an appropriate valence for said substitution.

In some embodiments, the compound of formula I-A, that binds to and modulates cereblon, and, in some instances, degrades IKZF2, is represented by formula VIII-A:

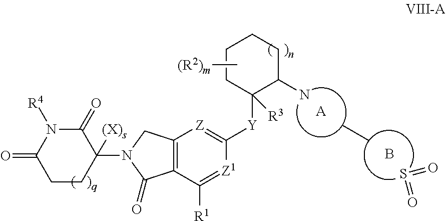

or a pharmaceutically acceptable salt, solvate, stereoisomer, or tautomer thereof, wherein ring A, ring B, R¹, R², R³, R⁴, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments of compounds of formula VIII-A,

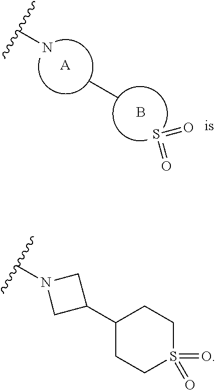

In some embodiments, the compound of formula VIII-A, that binds to and modulates cereblon, and, in some instances, degrades IKZF2, is represented by formula VIIIA-A:

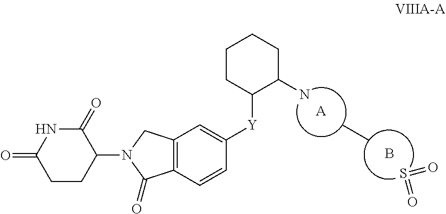

or a pharmaceutically acceptable salt, solvate, stereoisomer, or tautomer thereof, wherein Y, ring A, and ring B are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula VIII-A, that binds to and modulates cereblon, and, in some instances, degrades IKZF2, is represented by formula VIIIB-A:

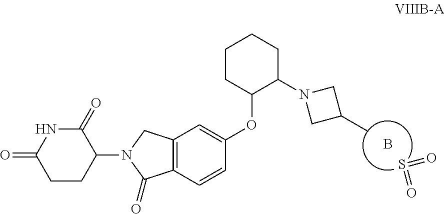

or a pharmaceutically acceptable salt, solvate, stereoisomer, or tautomer thereof, wherein ring B is as defined herein, and wherein ring B is independently unsubstituted or substituted with one to five R⁸.

It is to be understood that the independent substitution of ring B with one to five R⁸ substituents may occur at any location in ring B with an appropriate valence for said substitution.

In some embodiments, the compound of formula I-A, that binds to and modulates cereblon, and, in some instances, degrades IKZF2, is represented by formula IX-A:

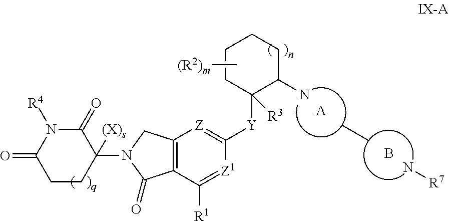

or a pharmaceutically acceptable salt, solvate, stereoisomer, or tautomer thereof, wherein ring A, ring B, R¹, R², R³, R⁴, R⁷, X, Y, Z, Z¹, m, n, q, and s are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

It is to be understood that the ring B nitrogen bearing the R⁷ group can be located at any position in ring B.

In some embodiments, for any compound of formula IX-A or sub-formulae thereof, or a pharmaceutically acceptable salt, solvate, stereoisomer, and/or tautomer thereof, R⁷ is hydrogen. In some embodiments, for any compound of formula IX-A or sub-formulae thereof, or a pharmaceutically acceptable salt, solvate, stereoisomer, and/or tautomer thereof, R⁷ is C₁-C₆ alkyl. In some embodiments, for any compound of formula IX-A or sub-formulae thereof, or a pharmaceutically acceptable salt, solvate, stereoisomer, and/or tautomer thereof, R⁷ is C₃-C₇ cycloalkyl.

In some embodiments of compounds of formula IX-A,

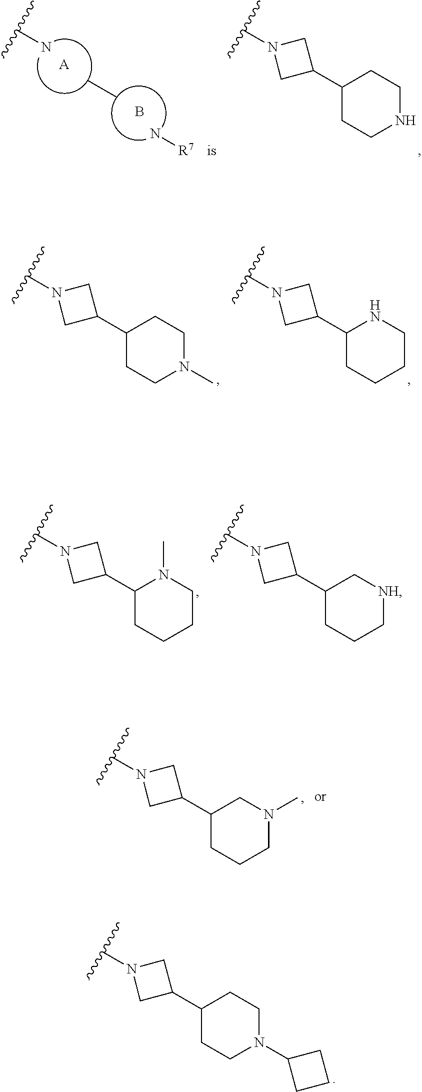

In some embodiments, the compound of formula IX-A, that binds to and modulates cereblon, and, in some instances, degrades IKZF2, is represented by formula IXA-A:

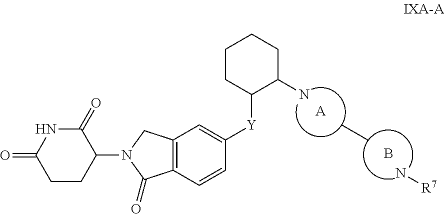

or a pharmaceutically acceptable salt, solvate, stereoisomer, or tautomer thereof, wherein Y, ring A, ring B, and R⁷ are as defined herein, and wherein ring A and ring B are independently unsubstituted or substituted with one to five R⁸.

It is to be understood that the substitution of ring A and ring B with independently one to five R⁸ substituents may occur at any location in ring A and ring B with an appropriate valence for said substitution.

In some embodiments, the compound of formula IX-A, that binds to and modulates cereblon, and, in some instances, degrades IKZF2, is represented by formula IXB-A:

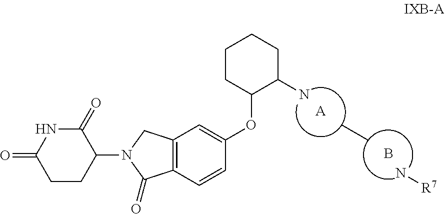

or a pharmaceutically acceptable salt, solvate, stereoisomer, or tautomer thereof, wherein ring B and R⁷ are as defined herein, and wherein ring B is independently unsubstituted or substituted with one to five R⁸.

It is to be understood that the independent substitution of ring B with one to five R⁸ substituents may occur at any location in ring B with an appropriate valence for said substitution.

In some embodiments, this disclosure provides a compound of formula IX-A, IXA-A, or IXB-A, or a pharmaceutically acceptable salt, solvate, stereoisomer, or tautomer thereof, wherein R⁷ is hydrogen, C₁-C₆ alkyl, or C₃-C₇ cycloalkyl.

In some embodiments, provided is a compound that binds to and modulates cereblon, and, in some instances, degrades IKZF2, of formula I-A′:

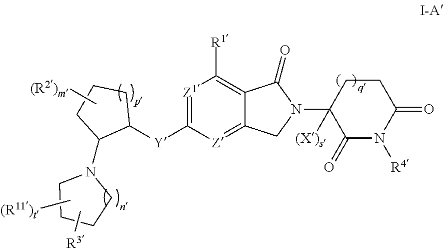

or a pharmaceutically acceptable salt, solvate, stereoisomer, and/or tautomer thereof,

wherein:

m′, n′, and p′ are independently zero, one, two, or three;

q′ is one, two, or three;

s′ is one;

t′ is zero or one;

X′ is hydrogen, deuterium, or fluoro;

Y′ is oxygen or NR′ where R′ is hydrogen or C₁-C₄ alkyl;

Z′ and Z^{1′} are each independently CR^{1′} or N;

each R^{1′} is independently selected from hydrogen, amino, (C₁-C₄ alkyl)amino independently unsubstituted or substituted with from one to three R^{5′} substituents, di-(C₁-C₄ alkyl)amino independently unsubstituted or substituted with from one to three R^{5′} substituents on each alkyl group, cyano, halo, hydroxyl, C₁-C₄ alkyl independently unsubstituted or substituted with from one to three R^{5′} substituents, and C₁-C₆ alkoxy independently unsubstituted or substituted with from one to three R^{5′} substituents; or

when Z^{1′} is CR^{1′}, then two adjacent R^{1′} together with the carbon atoms to which they are attached form a C₃-C₇ cycloalkyl, a C₆-C₁₀ aryl, a 4- to 7-membered heterocycloalkenyl having from one to three heteroatoms selected from oxygen, nitrogen, or sulfur, or a 5- to 6-membered heteroaryl having 1 to 3 heteroatoms selected from oxygen, nitrogen, and sulfur wherein each of said cycloalkyl, heterocycloalkenyl, aryl, and heteroaryl are independently substituted with one to three R^{6′} groups;

each R^{2′} is independently selected from cyano, halo, hydroxyl, amino, C₁-C₄ alkylamino independently unsubstituted or substituted with from one to three R^{5′} substituents, di-(C₁-C₄ alkyl)amino independently unsubstituted or substituted with from one to three R^{5′} substituents on each alkyl group, C₁-C₄ alkyl independently unsubstituted or substituted with from one to three R^{5′} substituents, and C₁-C₄ alkoxy independently unsubstituted or substituted with from one to three R^{5′} substituents;

R^{3′} is C₃-C₁₀ heterocycloalkyl or C₃-C₁₀ heterocycloalkenyl having from 1 to 3 heteroatoms selected from O, NR¹, and/or S and independently unsubstituted or substituted with 1 to 3 R^{7′} substituents;

R^{4′} is selected from hydrogen and —CH₂—OR^{8′} where R^{8′} is C(O)—R^{9′} or —P(O)(OR^{10″})₂, where R^{9′} is C₁-C₄ alkyl, or C₁-C₄ alkoxy, and where each R^{10′} is independently H or C₁-C₄ alkyl;

each R^{5′} is independently hydrogen, amino, (C₁-C₄ alkyl)amino, di-(C₁-C₄ alkyl)amino, cyano, halo, hydroxyl, or C₁-C₄ alkoxy;

each R^{6′} is independently selected from amino, (C₁-C₄ alkyl)amino, di-(C₁-C₄ alkyl)amino, cyano, halo, hydroxyl, and oxo;

each R^{7′} is independently selected from amino, C₁-C₄ alkyl unsubstituted or substituted with 1 to 3 halo, C₁-C₄ alkoxy, (C₁-C₄ alkyl)amino, di-(C₁-C₄ alkyl)amino, cyano, halo, hydroxyl, nitro, oxo, C₅-C₆ heteroaryl having from 1 to 3 heteroatoms selected from O, NR′, and/or S, and —C(CO)CH₃; and

R^{11′} is hydroxyl.

In some embodiments, the compound of formula I-A′, that binds to and modulates cereblon, and, in some instances, degrades IKZF2, has the structure of formula II-A′:

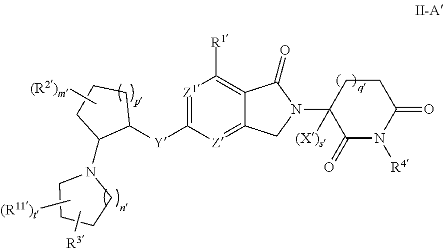

or a pharmaceutically acceptable salt, solvate, stereoisomer, and/or tautomer thereof wherein R^{1′}, R^{2′}, R^{3′}, R^{4′}, X′, Y′, Z′, Z^{1′}, m′, n′, p′, s′, and t′ are as defined herein.

In some embodiments, in a compound of formula I-A′ or formula II-A′, or a pharmaceutically acceptable salt, solvate, stereoisomer, and/or tautomer thereof, X is hydrogen or deuterium. In some embodiments, X′ is hydrogen. In some embodiments, X′ is deuterium. In some embodiments, X′ is tritium.

In some embodiments, in a compound of formula I-A′ or formula II-A′, or a pharmaceutically acceptable salt, solvate, stereoisomer, and/or tautomer thereof, X is fluoro.

In some embodiments, in a compound of formula I-A′ or formula II-A′, or a pharmaceutically acceptable salt, solvate, stereoisomer, and/or tautomer thereof, p′ is 1. In some embodiments, in a compound of formula I-A′ or formula II-A′, or a pharmaceutically acceptable salt, solvate, stereoisomer, and/or tautomer thereof, p′ is 2. In some embodiments, in a compound of formula I-A′ or formula II-A′, or a pharmaceutically acceptable salt, solvate, stereoisomer, and/or tautomer thereof, p′ is 3.

In some embodiments, in a compound of formula I-A′ or formula II-A′, or a pharmaceutically acceptable salt, solvate, stereoisomer, and/or tautomer thereof, n′ is 0. In some embodiments, in a compound of formula I-A′ or formula II-A′, or a pharmaceutically acceptable salt, solvate, stereoisomer, and/or tautomer thereof, n′ is 1. In some embodiments, in a compound of formula I-A′ or formula II-A′, or a pharmaceutically acceptable salt, solvate, stereoisomer, and/or tautomer thereof, n′ is 2. In some embodiments, in a compound of formula I-A′ or formula II-A′, or a pharmaceutically acceptable salt, solvate, stereoisomer, and/or tautomer thereof, n′ is 3.

In some embodiments, in a compound of formula I-A′ or formula II-A′, or a pharmaceutically acceptable salt, solvate, stereoisomer, and/or tautomer thereof, R^{4′} is hydrogen. In some embodiments, in a compound of formula I-A′ or formula II-A′, or a pharmaceutically acceptable salt, solvate, stereoisomer, and/or tautomer thereof, R^{4′} is —CH₂—O—C(O)—R^{9′} or —CH₂—O—P(O)(OR^{10′})₂. In some embodiments, in a compound of formula I-A′ or formula II-A′, or a pharmaceutically acceptable salt, solvate, stereoisomer, and/or tautomer thereof, R^{4′} is —CH₂—O—C(O)—CH₃, —CH₂—O—C(O)—CH₂CH₃, —CH₂—O—C(O)—CH₂CH₂CH₃, or —CH₂—O—C(O)—CH(CH₃)₂. In some embodiments, in a compound of formula I-A′ or formula II-A′, or a pharmaceutically acceptable salt, solvate, stereoisomer, and/or tautomer thereof, R^{4′} is —CH₂—O—P(O)(OCH₃)₂, —CH₂—O—P(O)(OCH₂CH₃)₂, —CH₂—O—P(O)(OCH₂CH₂CH₃)₂, or —CH₂—O—P(O)(O(CH(CH₃)₂)₂.

In some embodiments, in a compound of formula I-A′ or formula II-A′, or a pharmaceutically acceptable salt, solvate, stereoisomer, and/or tautomer thereof, Z′ and Z^{1′} are each C—R^{1′}. In some of such embodiments, Z′ and Z^{1′} are each C—H. In some embodiments, in a compound of formula I-A′ or formula II-A′, or a pharmaceutically acceptable salt, solvate, stereoisomer, and/or tautomer thereof, Z′ and Z^{1′} are each C—R^{1′}, wherein one R^{1′} is hydrogen, and the other R^{1′} is C₁-C₆ alkoxy. In some embodiments, in a compound of formula I-A′ or formula II-A′, or a pharmaceutically acceptable salt, solvate, stereoisomer, and/or tautomer thereof, Z′ and Z″ are each C—R^{1′}, wherein one R^{1′} is hydrogen, and the other R^{1′} is a halogen, such as bromo, chloro, or fluoro. In some embodiments, in a compound of formula I-A′ or formula II-A′, or a pharmaceutically acceptable salt, solvate, stereoisomer, and/or tautomer thereof, Z′ and Z^{1′} are each N. In some embodiments, in a compound of formula I-A′ or formula II-A′, or a pharmaceutically acceptable salt, solvate, stereoisomer, and/or tautomer thereof, one of Z′ or Z^{1′} is C—R^{1′} and the other of Z′ and Z^{1′} is N. In some of such embodiments, one of Z′ or Z^{1′} is C—H and the other of Z′ or Z^{1′} is N. In some embodiments, in a compound of formula I-A′ or formula II-A′, or a pharmaceutically acceptable salt, solvate, stereoisomer, and/or tautomer thereof, R^{1′} is H.

In some embodiments, in a compound of formula I-A′ or formula II-A′, or a pharmaceutically acceptable salt, solvate, stereoisomer, and/or tautomer thereof, m′ is zero. In some embodiments, in a compound of formula I-A′ or formula II-A′, or a pharmaceutically acceptable salt, solvate, stereoisomer, and/or tautomer thereof, m′ is 1. In some embodiments, in a compound of formula I-A′ or formula II-A′, or a pharmaceutically acceptable salt, solvate, stereoisomer, and/or tautomer thereof, m′ is 2.

In some embodiments, in a compound of formula I-A′ or formula II-A′, or a pharmaceutically acceptable salt, solvate, stereoisomer, and/or tautomer thereof, q′ is 1. In some embodiments, in a compound of formula I-A′ or formula II-A′, or a pharmaceutically acceptable salt, solvate, stereoisomer, and/or tautomer thereof, q′ is 2. In some embodiments, in a compound of formula I-A′ or formula II-A′, or a pharmaceutically acceptable salt, solvate, stereoisomer, and/or tautomer thereof, q′ is 3.

In some embodiments, a compound of formula I-A′, that binds to and modulates cereblon, and, in some instances, degrades IKZF2, has the structure of formula III-A′:

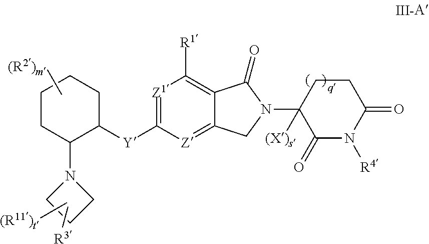

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, q′, s′, and t′ are as defined herein. In some embodiments of formula III-A′, Y′ is O. In some embodiments of formula III-A′, Y′ is NR′. In some embodiments of formula III-A′, Z′ and Z^{1′} are each C—H.

In some embodiments, a compound of formula III-A′, that binds to and modulates cereblon, and, in some instances, degrades IKZF2, has the structure of formula IV-A′:

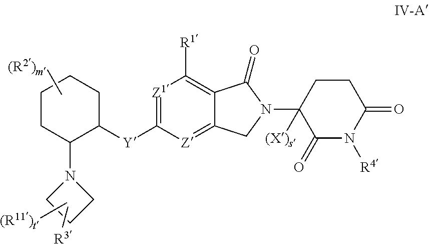

or a pharmaceutically acceptable salt, solvate, stereoisomer, and/or tautomer thereof, wherein R^{1′}, R^{2′}, R^{3′}, R^{4′}, X′, Y′, Z′, Z^{1′}, m′, s′, and t′ are as defined herein. In some embodiments of formula IV-A′, Y′ is O. In some embodiments of formula IV-A′, Y′ is NR′. In some embodiments of formula IV-A′, Z′ and Z^{1′} are each C—H.

In some embodiments, a compound of formula I-A′, that binds to and modulates cereblon, and, in some instances, degrades IKZF2, has the structure of formula V-A′:

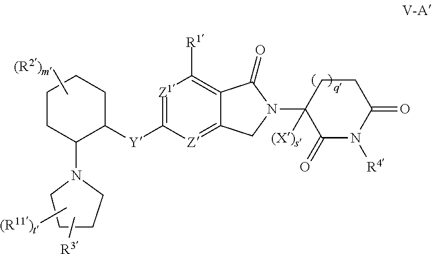

or a pharmaceutically acceptable salt, solvate, stereoisomer, and/or tautomer thereof wherein R^{1′}, R^{2′}, R^{3′}, R^{4′}, X′, Y′, Z′, Z^{1′}, m′, q′, s′, and t′ are as defined herein. In some embodiments of formula V-A′, Y′ is O. In some embodiments of formula V-A′, Y′ is NR′. In some embodiments of formula V-A′, Z′ and Z^{1′} are each C—H.

In some embodiments, a compound of formula V-A′, that binds to and modulates cereblon, and, in some instances, degrades IKZF2, has the structure of formula VI-A′:

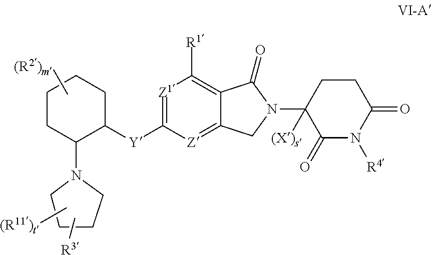

or a pharmaceutically acceptable salt, solvate, stereoisomer, and/or tautomer thereof wherein R^{1′}, R^{2′}, R^{3′}, R^{4′}, X′, Y′, Z′, Z^{1′}, m′, s′, and t′ are as defined herein. In some embodiments of formula VI-A′, Y′ is O. In some embodiments of formula VI-A′, Y is NR′. In some embodiments of formula VI-A′, Z′ and Z^{1′} are each C—H.

In some embodiments, for any compound of formula I-A′ or sub-formulae thereof, or a pharmaceutically acceptable salt, solvate, stereoisomer, and/or tautomer thereof,

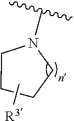

is selected from

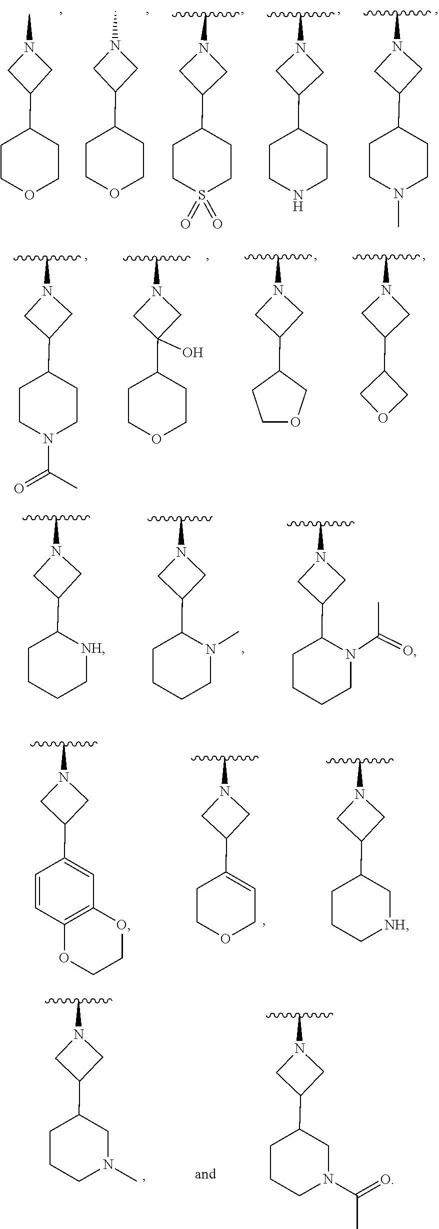

In some embodiments, for any compound of formula I-A′ or sub-formulae thereof, or a pharmaceutically acceptable salt, solvate, stereoisomer, and/or tautomer thereof, Y′ is O. In some embodiments, for any compound of formula I-A′ or sub-formulae thereof, or a pharmaceutically acceptable salt, solvate, stereoisomer, and/or tautomer thereof, Y′ is NR′. In some embodiments, for any compound of formula I-A′ or sub-formulae thereof, or a pharmaceutically acceptable salt, solvate, stereoisomer, and/or tautomer thereof, R^{2′} is halo, e.g., fluoro. In some embodiments, for any compound of formula I-A′ or sub-formulae thereof, or a pharmaceutically acceptable salt, solvate, stereoisomer, and/or tautomer thereof, R^{2′} is C₁-C₄ alkyl, e.g., methyl. In some embodiments, for any compound of formula I-A′ or sub-formulae thereof, or a pharmaceutically acceptable salt, solvate, stereoisomer, and/or tautomer thereof, t′ is zero. In some embodiments, for any compound of formula I-A′ or sub-formulae thereof, or a pharmaceutically acceptable salt, solvate, stereoisomer, and/or tautomer thereof, t′ is 1 and R^{11′} is hydroxyl.

In some embodiments, provided herein is a compound which binds to and modulates cereblon, and, in some instances, degrades IKZF2, of formula I-B:

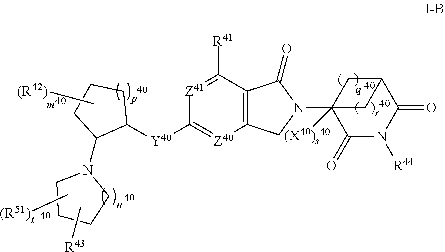

or a pharmaceutically acceptable salt, solvate, stereoisomer, and/or tautomer thereof,
wherein:

m⁴⁰, n⁴⁰ and p⁴⁰ are independently zero, one, two, or three;

q⁴⁰ is one, two or three;

r⁴⁰ is zero, one, or two;

s⁴⁰ is zero when r⁴⁰ is not zero and is one when r⁴⁰ is zero;

t⁴⁰ is zero or one;

X⁴⁰ is hydrogen, deuterium, or fluoro;

Y⁴⁰ is oxygen or NR⁴⁰ where R⁴⁰ is hydrogen or C₁-C₄ alkyl;

Z⁴⁰ and Z⁴¹ are each independently CR⁴¹ or N;

each R⁴¹ is independently selected from hydrogen, amino, (C₁-C₄ alkyl)amino unsubstituted or substituted with from one to three R⁴⁵ substituents, di-(C₁-C₄ alkyl)amino unsubstituted or substituted with from one to three R⁴⁵ substituents on each alkyl group, cyano, halo, hydroxyl, C₁-C₄ alkyl unsubstituted or substituted with from one to three R⁴⁵ substituents, and C₁-C₄ alkoxy unsubstituted or substituted with from one to three R⁴⁵ substituents; or

when Z⁴¹ is CR⁴¹, then two adjacent R⁴¹ together with the carbon atoms to which they are attached form a C₃-C₇ cycloalkyl, a C₆-C₁₀ aryl, a 4- to 7-membered heterocycloalkenyl having from one to three heteroatoms selected from oxygen, nitrogen, or sulfur, or a 5- to 6-membered heteroaryl having 1 to 3 heteroatoms selected from oxygen, nitrogen, or sulfur wherein each of said cycloalkyl, heterocycloalkenyl, aryl, and heteroaryl are independently substituted with one to three R⁴⁶ groups;

each R⁴² is independently selected from cyano, halo, hydroxyl, amino, C₁-C₄ alkylamino unsubstituted or substituted with from one to three R⁴⁵ substituents, di-(C₁-C₄ alkyl)amino unsubstituted or substituted with from one to three R⁴⁵ substituents on each alkyl group, C₁-C₄ alkyl unsubstituted or substituted with from one to three R⁴⁵ substituents, and C₁-C₄ alkoxy unsubstituted or substituted with from one to three R⁴⁵ substituents;

R⁴³ is C₅-C₁₀ heteroaryl having from 1 to 3 heteroatoms selected from O, N, and/or S and unsubstituted or substituted with 1 to 3 R⁴⁷ substituents;

R⁴⁴ is selected from hydrogen and —CH₂—OR⁴⁸ where R⁴⁸ is C(O)—R⁴⁹ or —P(O)(OR⁵⁰)₂, where R⁴⁹ is C₁-C₄ alkyl or C₁-C₄ alkoxy, and where each of R⁵⁰ is independently H or C₁-C₄ alkyl;

each R⁴⁵ is independently amino, (C₁-C₄ alkyl)amino, di-(C₁-C₄ alkyl)amino, cyano, halo, hydroxyl, or C₁-C₄ alkoxy;

each R⁴⁶ is independently selected from amino, (C₁-C₄ alkyl)amino, di-(C₁-C₄ alkyl)amino, cyano, halo, hydroxyl, and oxo;

each R⁴⁷ is independently selected from amino, C₁-C₄ alkyl unsubstituted or substituted with 1 to 3 halo, C₁-C₄ alkoxy, (C₁-C₄ alkyl)amino, di-(C₁-C₄ alkyl)amino, cyano, halo, hydroxyl, nitro, C₅-C₆ heteroaryl having from 1 to 3 heteroatoms selected from O, NR⁴⁰, and/or S, C₃-C₇ cycloalkyl, and —C(O)CH₃; and

R⁵¹ is hydroxyl, cyano, halo, or C₁-C₄ alkyl unsubstituted or substituted with 1 to 3 halo.

In some embodiments, the compound which binds to and modulates cereblon, and, in some instances, degrades IKZF2, of formula I-B has the structure of formula II-B:

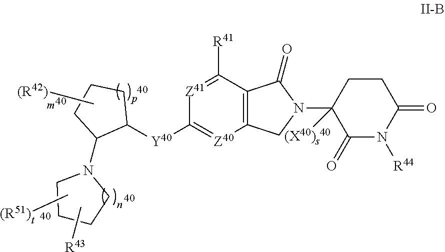

or a pharmaceutically acceptable salt, solvate, stereoisomer, and/or tautomer thereof wherein R⁴¹, R⁴², R⁴³, R⁴⁴, R⁵¹, X⁴⁰, Y⁴⁰, Z⁴⁰, Z⁴¹, m⁴⁰, n⁴⁰, p⁴⁰, s⁴⁰, and t⁴⁰ are as defined herein.

In some embodiments, in a compound of formula I-B or formula II-B, or a pharmaceutically acceptable salt, solvate, stereoisomer, and/or tautomer thereof, X⁴⁰ is hydrogen or deuterium. In some embodiments, X⁴⁰ is hydrogen. In some embodiments, X⁴⁰ is deuterium. In some embodiments, X⁴⁰ is tritium.

In some embodiments, in a compound of formula I-B or formula II-B, or a pharmaceutically acceptable salt, solvate, stereoisomer, and/or tautomer thereof, X⁴⁰ is fluoro.

In some embodiments, in a compound of formula I-B or formula II-B, or a pharmaceutically acceptable salt, solvate, stereoisomer, and/or tautomer thereof, p⁴⁰ is 1. In some embodiments, in a compound of formula I-B or formula II-B, or a pharmaceutically acceptable salt, solvate, stereoisomer, and/or tautomer thereof, p⁴⁰ is 2. In some embodiments, in a compound of formula I-B or formula II-B, or a pharmaceutically acceptable salt, solvate, stereoisomer, and/or tautomer thereof, p⁴⁰ is 3.

In some embodiments, in a compound of formula I-B or formula II-B, or a pharmaceutically acceptable salt, solvate, stereoisomer, and/or tautomer thereof, n⁴⁰ is 0. In some embodiments, in a compound of formula I-B or formula II-B, or a pharmaceutically acceptable salt, solvate, stereoisomer, and/or tautomer thereof, n⁴⁰ is 1. In some embodiments, in a compound of formula I-B or formula II-B, or a pharmaceutically acceptable salt, solvate, stereoisomer, and/or tautomer thereof, n⁴⁰ is 2. In some embodiments, in a compound of formula I-B or formula II-B, or a pharmaceutically acceptable salt, solvate, stereoisomer, and/or tautomer thereof, n⁴⁰ is 3.

In some embodiments, in a compound of formula I-B or formula II-B, or a pharmaceutically acceptable salt, solvate, stereoisomer, and/or tautomer thereof, R⁴⁴ is hydrogen. In some embodiments, in a compound of formula I-B or formula II-B, or a pharmaceutically acceptable salt, solvate, stereoisomer, and/or tautomer thereof, R⁴⁴ is —CH₂—O—C(O)—R⁴⁹ or —CH₂—O—P(O)(OR⁵⁰)₂. In some embodiments, in a compound of formula I-B or formula II-B, or a pharmaceutically acceptable salt, solvate, stereoisomer, and/or tautomer thereof, R⁴⁴ is —CH₂—O—C(O)—CH₃, —CH₂—O—C(O)—CH₂CH₃, —CH₂—O—C(O)—CH₂CH₂CH₃, or —CH₂—O—C(O)—CH(CH₃)₂. In some embodiments, in a compound of formula I-B or formula II-B, or a pharmaceutically acceptable salt, solvate, stereoisomer, and/or tautomer thereof, R⁴⁴ is —CH₂—O—P(O)(OCH₃)₂, —CH₂—O—P(O)(OCH₂CH₃)₂, —CH₂—O—P(O)(OCH₂CH₂CH₃)₂, or —CH₂—O—P(O)(O(CH(CH₃)₂)₂.

In some embodiments, in a compound of formula I-B or formula II-B, or a pharmaceutically acceptable salt, solvate, stereoisomer, and/or tautomer thereof, Z⁴⁰ and Z⁴¹ are each C—R⁴¹. In some of such embodiments, Z⁴⁰ and Z⁴¹ are each C—H. In some embodiments, in a compound of formula I-B or formula II-B, or a pharmaceutically acceptable salt, solvate, stereoisomer, and/or tautomer thereof, Z⁴⁰ and Z⁴¹ are each N. In some embodiments, in a compound of formula I-B or formula II-B, or a pharmaceutically acceptable salt, solvate, stereoisomer, and/or tautomer thereof, one of Z⁴⁰ or Z⁴¹ is C—R⁴¹ and the other of Z⁴⁰ or Z⁴¹ is N. In some of such embodiments, one of Z⁴⁰ or Z⁴¹ is C—H and the other of Z⁴⁰ or Z⁴¹ is N. In some embodiments, in a compound of formula I-B or formula II-B, or a pharmaceutically acceptable salt, solvate, stereoisomer, and/or tautomer thereof, R⁴¹ is H.

In some embodiments, in a compound of formula I-B or formula II-B, or a pharmaceutically acceptable salt, solvate, stereoisomer, and/or tautomer thereof, R⁵¹ is hydroxyl. In some embodiments, in a compound of formula I-B or formula II-B, or a pharmaceutically acceptable salt, solvate, stereoisomer, and/or tautomer thereof, R⁵¹ is cyano. In some embodiments, in a compound of formula I-B or formula II-B, or a pharmaceutically acceptable salt, solvate, stereoisomer, and/or tautomer thereof, R⁵¹ is halo. In some of such embodiments, R⁵¹ is fluoro. In some embodiments, in a compound of formula I-B or formula II-B, or a pharmaceutically acceptable salt, solvate, stereoisomer, and/or tautomer thereof, R⁵¹ is C₁-C₄ alkyl unsubstituted or substituted with 1 to 3 halo. In some of such embodiments, R⁵¹ is difluoromethyl. In other of such embodiments, R⁵¹ is trifluoromethyl.

In some embodiments, in a compound of formula I-B or formula II-B, or a pharmaceutically acceptable salt, solvate, stereoisomer, and/or tautomer thereof, m⁴⁰ is zero. In some embodiments, in a compound of formula I-B or formula II-B, or a pharmaceutically acceptable salt, solvate, stereoisomer, and/or tautomer thereof, m⁴⁰ is 1. In some embodiments, in a compound of formula I-B or formula II-B, or a pharmaceutically acceptable salt, solvate, stereoisomer, and/or tautomer thereof, m⁴⁰ is 2.

In some embodiments, in a compound of formula I-B or formula II-B, or a pharmaceutically acceptable salt, solvate, stereoisomer, and/or tautomer thereof, q⁴⁰ is 1, and r⁴⁰ is 1. In some embodiments, in a compound of formula I-B or formula II-B, or a pharmaceutically acceptable salt, solvate, stereoisomer, and/or tautomer thereof, q⁴⁰ is 1 and r⁴⁰ is 0.

In some embodiments, a compound of formula I-B which binds to and modulates cereblon, and, in some instances, degrades IKZF2, has the structure of formula III-B:

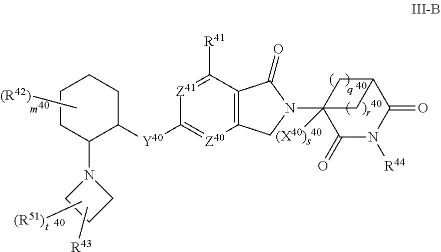

or a pharmaceutically acceptable salt, solvate, stereoisomer, and/or tautomer thereof wherein R⁴¹, R⁴², R⁴³, R⁴⁴, R⁵¹, X⁴⁰, Y⁴⁰, Z⁴⁰, Z⁴¹, m⁴⁰, q⁴⁰, r⁴⁰, s⁴⁰, and t⁴⁰ are as defined herein. In some embodiments of formula III-B, Y⁴⁰ is O. In some embodiments of formula III-B, Y⁴⁰ is NR⁴⁰. In some embodiments of formula III-B, Z⁴⁰ and Z⁴¹ are each C—H.

In some embodiments, a compound of formula III-B which binds to and modulates cereblon, and, in some instances, degrades IKZF2, has the structure of formula IV-B:

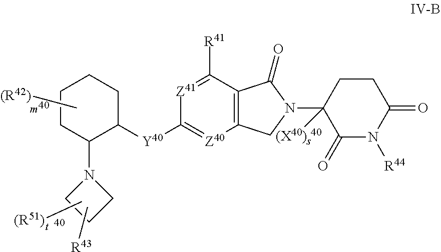

or a pharmaceutically acceptable salt, solvate, stereoisomer, and/or tautomer thereof wherein R⁴¹, R⁴², R⁴³, R⁴⁴, R⁵¹, X⁴⁰, Y⁴⁰, Z⁴⁰, Z⁴¹, m⁴⁰, s⁴⁰, and t⁴⁰ are as defined herein. In some embodiments of formula IV-B, Y⁴⁰ is O. In some embodiments of formula IV-B, Y⁴⁰ is NR⁴⁰. In some embodiments of formula IV-B, Z⁴⁰ and Z⁴¹ are each C—H.

In some embodiments, a compound of formula I-B which binds to and modulates cereblon, and, in some instances, degrades IKZF2, has the structure of formula V-B:

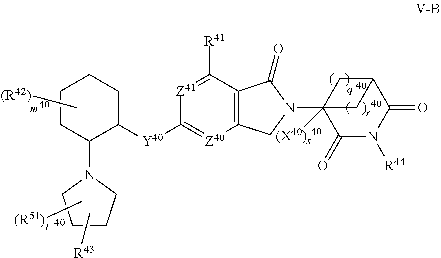

or a pharmaceutically acceptable salt, solvate, stereoisomer, and/or tautomer thereof wherein R⁴¹, R⁴², R⁴³, R⁴⁴, R⁵¹, X⁴⁰, Y⁴⁰, Z⁴⁰, Z⁴¹, m⁴⁰, q⁴⁰, r⁴⁰, s⁴⁰, and t⁴⁰ are as defined herein. In some embodiments of formula V-B, Y⁴⁰ is O. In some embodiments of formula V-B, Y⁴⁰ is NR⁴⁰. In some embodiments of formula V-B, Z⁴⁰ and Z⁴¹ are each C—H.

In some embodiments, a compound of formula V-B which binds to and modulates cereblon, and, in some instances, degrades IKZF2, has the structure of formula VI-B:

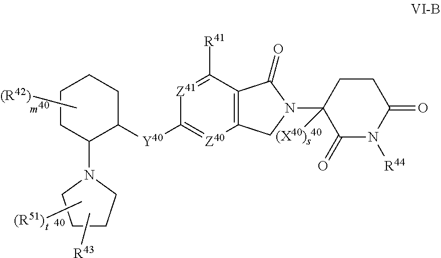

or a pharmaceutically acceptable salt, solvate, stereoisomer, and/or tautomer thereof wherein R⁴¹, R⁴², R⁴³, R⁴⁴, R⁵¹, X⁴⁰, Y⁴⁰, Z⁴⁰, Z⁴¹, m⁴⁰, s⁴⁰, and t⁴⁰ are as defined herein. In some embodiments of formula VI-B, Y⁴⁰ is O. In some embodiments of formula VI-B, Y⁴⁰ is NR⁴⁰. In some embodiments of formula VI-B, Z⁴⁰ and Z⁴¹ are each C—H.

In some embodiments, for any compound of formula I-B or sub-formulae thereof, or a pharmaceutically acceptable salt, solvate, stereoisomer, and/or tautomer thereof,

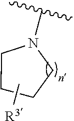

is selected from

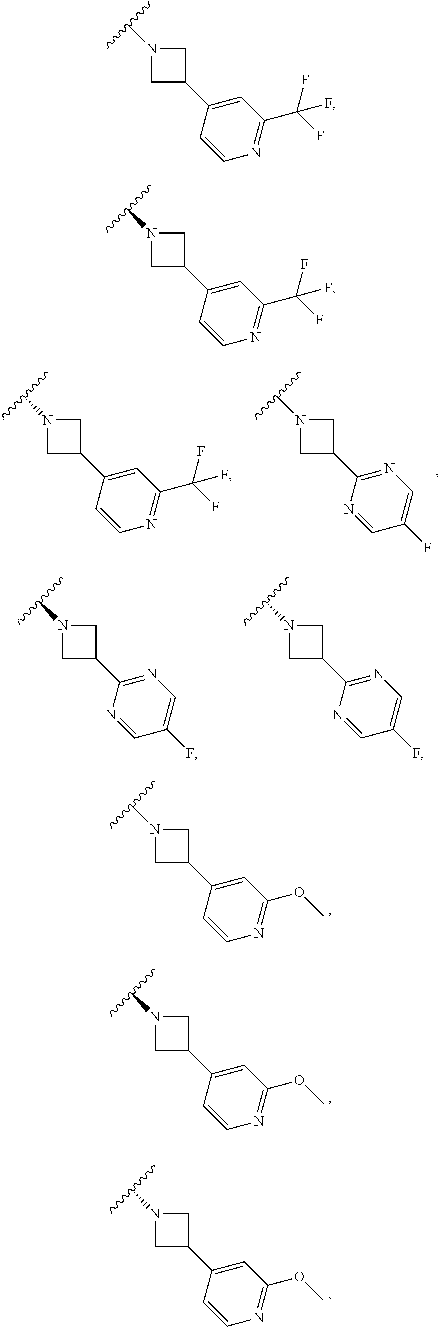

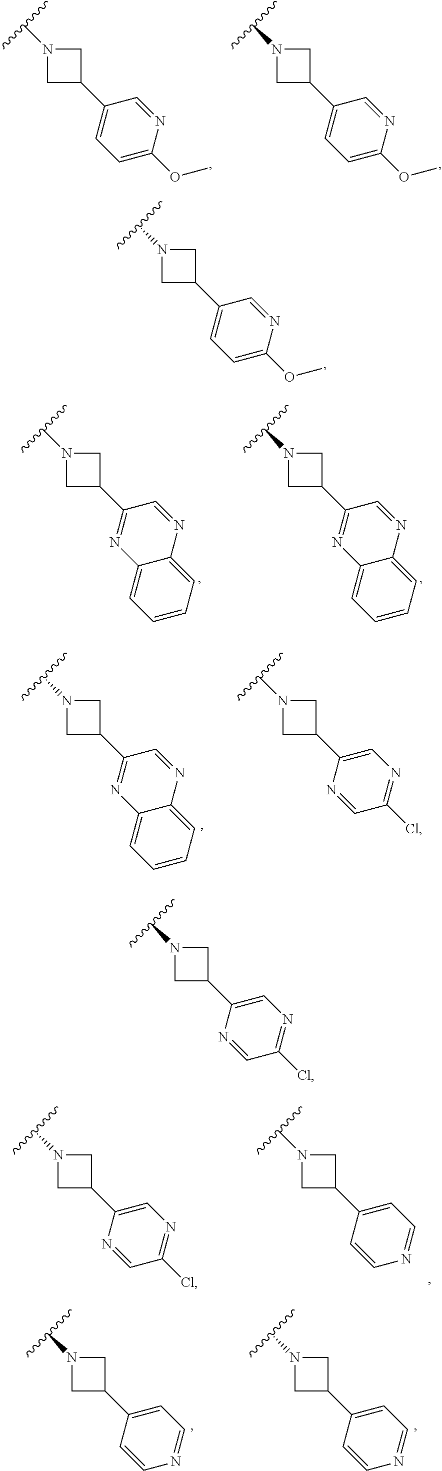

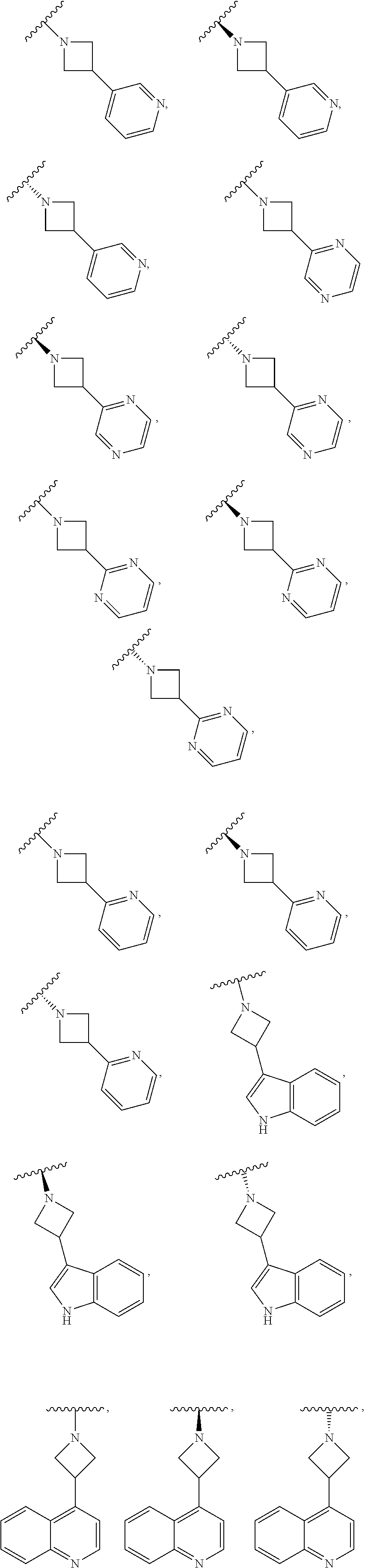

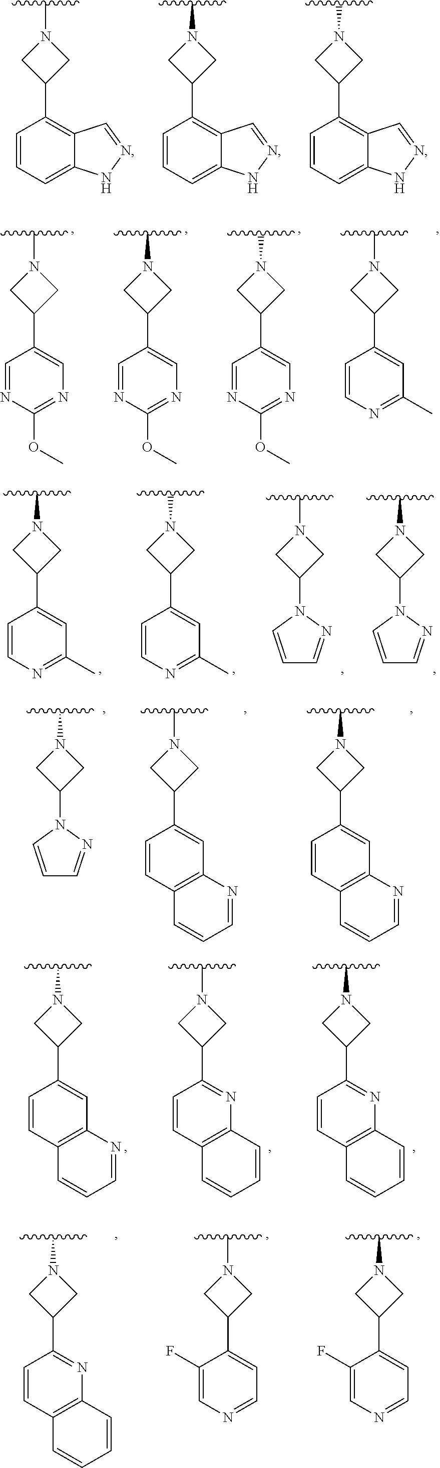

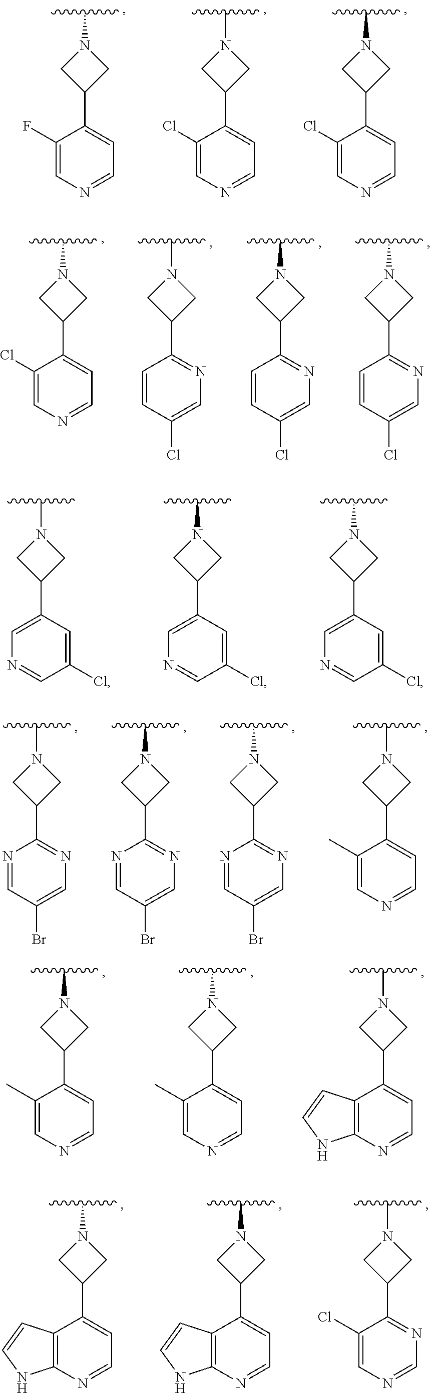

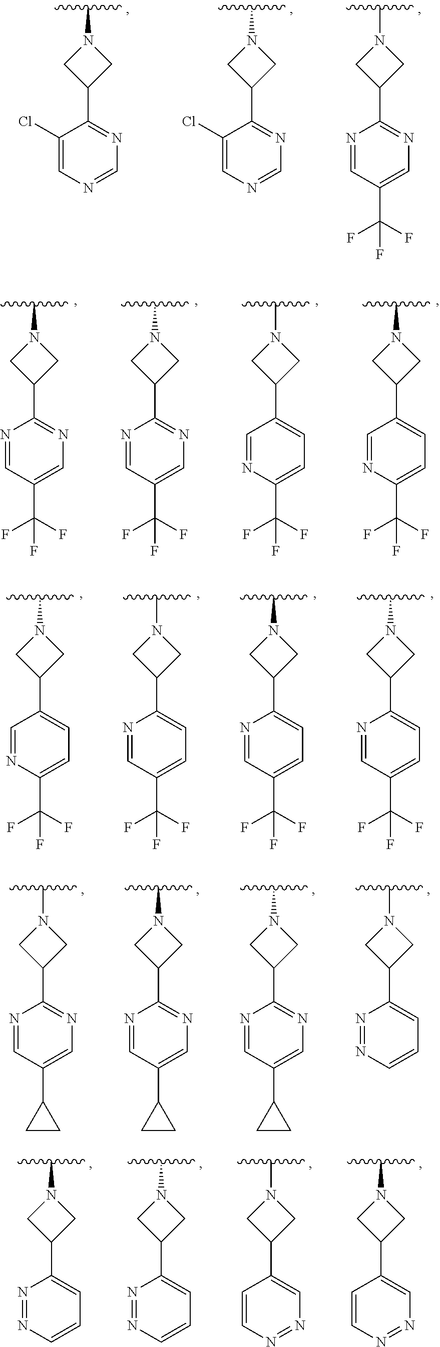

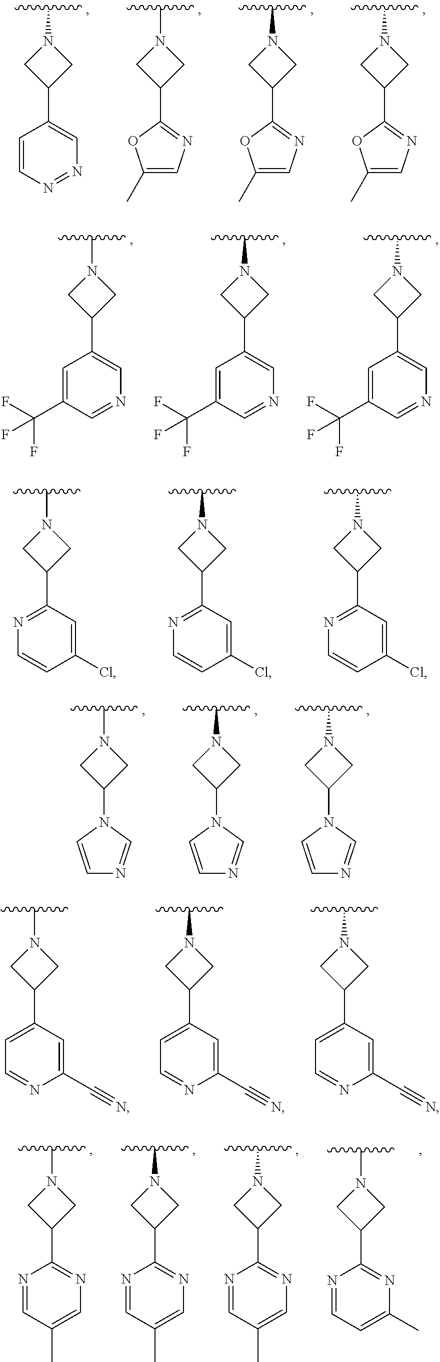

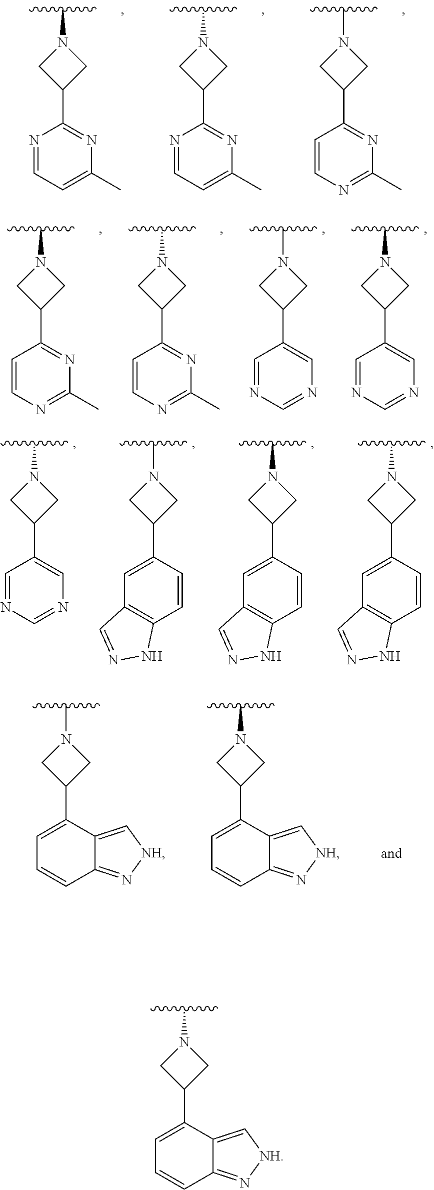

In some embodiments, for any compound of formula I-B or sub-formulae thereof, or a pharmaceutically acceptable salt, solvate, stereoisomer, and/or tautomer thereof, where q⁴⁰ is one, two, or three, and r⁴⁰ is one or two, the moiety

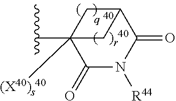

comprises a bridged ring system. In some of such embodiments, q⁴⁰ is one, r⁴⁰ is one, and s⁴⁰ is zero, and the moiety

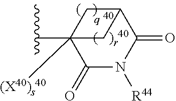

comprises a bridged ring system. In some embodiments, for any compound of formula I-B or sub-formulae thereof, or a pharmaceutically acceptable salt, solvate, stereoisomer, and/or tautomer thereof, where r⁴⁰ is zero, the moiety

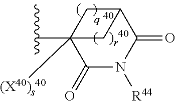

comprises a monocyclic ring and s⁴⁰ is one.

In some embodiments, for any compound of formula I-B or sub-formulae thereof, or a pharmaceutically acceptable salt, solvate, stereoisomer, and/or tautomer thereof, Y⁴⁰ is O. In some embodiments, for any compound of formula I-B or sub-formulae thereof, or a pharmaceutically acceptable salt, solvate, stereoisomer, and/or tautomer thereof, Y⁴⁰ is NR⁴⁰. In some embodiments, for any compound of formula I-B or sub-formulae thereof, or a pharmaceutically acceptable salt, solvate, stereoisomer, and/or tautomer thereof, R⁴² is halo, e.g., fluoro. In some embodiments, for any compound of formula I-B or sub-formulae thereof, or a pharmaceutically acceptable salt, solvate, stereoisomer, and/or tautomer thereof, R⁴² is C₁-C₄ alkyl, e.g., methyl. In some embodiments, for any compound of formula I-B or sub-formulae thereof, or a pharmaceutically acceptable salt, solvate, stereoisomer, and/or tautomer thereof, t⁴⁰ is zero. In some embodiments, for any compound of formula I-B or sub-formulae thereof, or a pharmaceutically acceptable salt, solvate, stereoisomer, and/or tautomer thereof, t⁴⁰ is 1 and R⁵¹ is hydroxyl.

In some embodiments, provided herein is a compound selected from Table 1, or a pharmaceutically acceptable salt, solvate, stereoisomer, and/or tautomer thereof.

TABLE 1

| # | Structure | Nomenclature |
|---|---|---|
| 1-A | 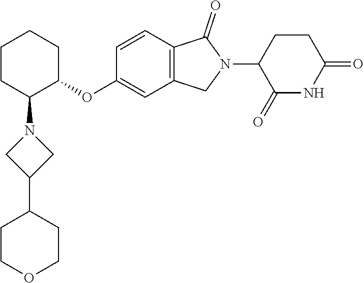 [SMILES: O=C1CCC(N2Cc3cc(O[C@H]4CCCC[C@@H]4N4CC(C5CCOCC5)C4)ccc3C2=O)C(=O)N1] | 3-(1-oxo-5-(((1S,2S)-2-(3- (tetrahydro-2H-pyran-4-yl)azetidin- 1-yl)cyclohexyl)oxy)isoindolin-2- yl)piperidine-2,6-dione |
| 2-A | 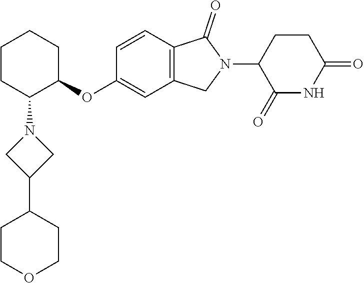 [SMILES: O=C1CCC(N2Cc3cc(O[C@@H]4CCCC[C@H]4N4CC(C5CCOCC5)C4)ccc3C2=O)C(=O)N1] | 3-(1-oxo-5-(((1R,2R)-2-(3- (tetrahydro-2H-pyran-4-yl)azetidin- 1-yl)cyclohexyl)oxy)isoindolin-2- yl)piperidine-2,6-dione |
| 3-A | 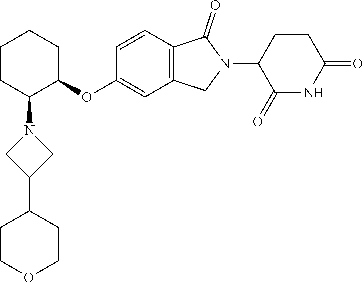 [SMILES: O=C1CCC(N2Cc3cc(O[C@@H]4CCCC[C@@H]4N4CC(C5CCOCC5)C4)ccc3C2=O)C(=O)N1] | 3-(1-oxo-5-(((1R,2S)-2-(3- (tetrahydro-2H-pyran-4-yl)azetidin- 1-yl)cyclohexyl)oxy)isoindolin-2- yl)piperidine-2,6-dione |
| 4-A | 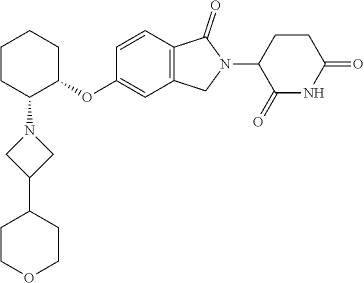 [SMILES: O=C1CCC(N2Cc3cc(O[C@H]4CCCC[C@H]4N4CC(C5CCOCC5)C4)ccc3C2=O)C(=O)N1] | 3-(1-oxo-5-(((1S,2R)-2-(3- (tetrahydro-2H-pyran-4-yl)azetidin- 1-yl)cyclohexyl)oxy)isoindolin-2- yl)piperidine-2,6-dione |
| 5-A | 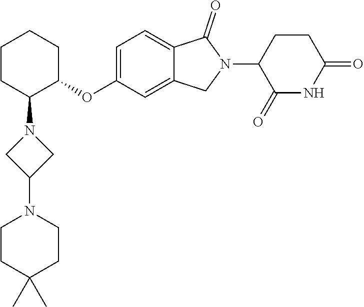 [SMILES: CC1(C)CCN(C2CN([C@H]3CCCC[C@@H]3Oc3ccc4c(c3)CN(C3CCC(=O)NC3=O)C4=O)C2)CC1] | 3-(5-(((1S,2S)-2-(3-(4,4- dimethylpiperidin-1-yl)azetidin-1- yl)cyclohexyl)oxy)-1- oxoisoindolin-2-yl)piperidine-2,6- dione |
| 6-A | 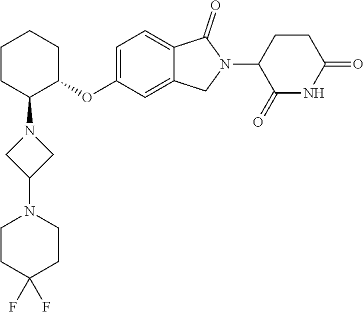 [SMILES: O=C1CCC(N2Cc3cc(O[C@H]4CCCC[C@@H]4N4CC(N5CCC(F)(F)CC5)C4)ccc3C2=O)C(=O)N1] | 3-(5-(((1S,2S)-2-(3-(4,4- difluoropiperidin-1-yl)azetidin-1- yl)cyclohexyl)oxy)-1- oxoisoindolin-2-yl)piperidine-2,6- dione |
| 7-A | 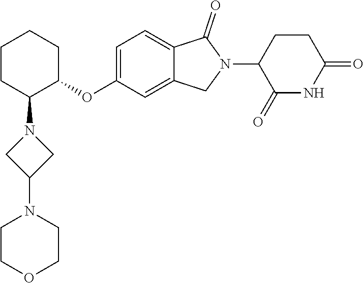 [SMILES: O=C1CCC(N2Cc3cc(O[C@H]4CCCC[C@@H]4N4CC(N5CCOCC5)C4)ccc3C2=O)C(=O)N1] | 3-(5-(((1S,2S)-2-(3- morpholinoazetidin-1- yl)cyclohexyl)oxy)-1- oxoisoindolin-2-yl)piperidine-2,6- dione |
| 8-A | 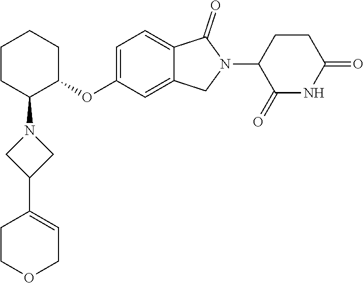 [SMILES: O=C1CCC(N2Cc3cc(O[C@H]4CCCC[C@@H]4N4CC(C5=CCOCC5)C4)ccc3C2=O)C(=O)N1] | 3-(5-(((1S,2S)-2-(3-(3,6-dihydro- 2H-pyran-4-yl)azetidin-1- yl)cyclohexyl)oxy)-1- oxoisoindolin-2-yl)piperidine-2,6- dione |
| 9-A | 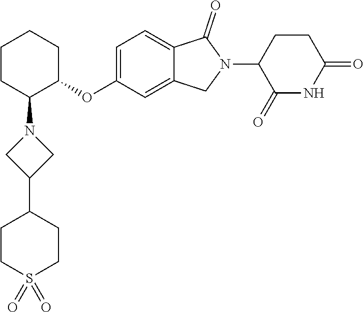 [SMILES: O=C1CCC(N2Cc3cc(O[C@H]4CCCC[C@@H]4N4CC(C5CCS(=O)(=O)CC5)C4)ccc3C2=O)C(=O)N1] | 3-(5-(((1S,2S)-2-(3-(1,1- dioxidotetrahydro-2H-thiopyran-4- yl)azetidin-1-yl)cyclohexyl)oxy)-1- oxoisoindolin-2-yl)piperidine-2,6- dione |
| 10-A | 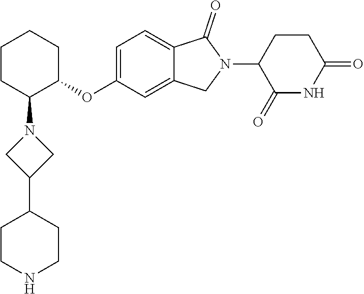 [SMILES: O=C1CCC(N2Cc3cc(O[C@H]4CCCC[C@@H]4N4CC(C5CCNCC5)C4)ccc3C2=O)C(=O)N1] | 3-(1-oxo-5-(((1S,2S)-2-(3- (piperidin-4-yl)azetidin-1- yl)cyclohexyl)oxy)isoindolin-2- yl)piperidine-2,6-dione |
| 11-A | 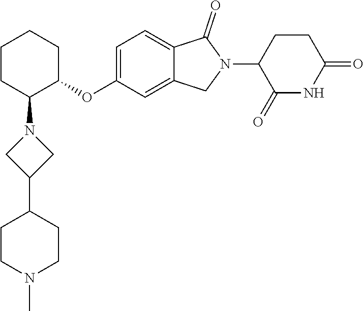 [SMILES: CN1CCC(C2CN([C@H]3CCCC[C@@H]3Oc3ccc4c(c3)CN(C3CCC(=O)NC3=O)C4=O)C2)CC1] | 3-(5-(((1S,2S)-2-(3-(1- methylpiperidin-4-yl)azetidin-1- yl)cyclohexyl)oxy)-1- oxoisoindolin-2-yl)piperidine-2,6- dione |
| 12-A | 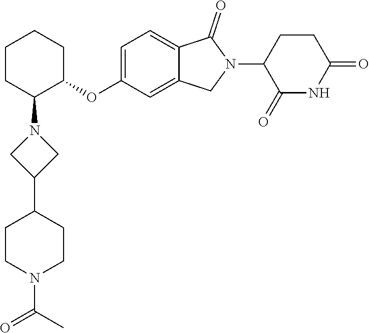 [SMILES: CC(=O)N1CCC(C2CN([C@H]3CCCC[C@@H]3Oc3ccc4c(c3)CN(C3CCC(=O)NC3=O)C4=O)C2)CC1] | 3-(5-(((1S,2S)-2-(3-(1- acetylpiperidin-4-yl)azetidin-1- yl)cyclohexyl)oxy)-1- oxoisoindolin-2-yl)piperidine-2,6- dione |
| 13-A | 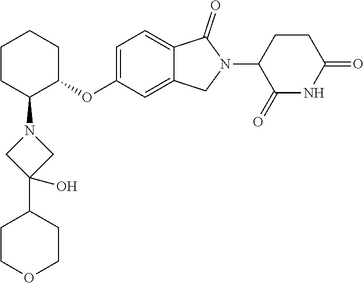 [SMILES: O=C1CCC(N2Cc3cc(O[C@H]4CCCC[C@@H]4N4CC(O)(C5CCOCC5)C4)ccc3C2=O)C(=O)N1] | 3-(5-(((1S,2S)-2-(3-hydroxy-3- (tetrahydro-2H-pyran-4-yl)azetidin- 1-yl)cyclohexyl)oxy)-1- oxoisoindolin-2-yl)piperidine-2,6- dione |
| 14-A | 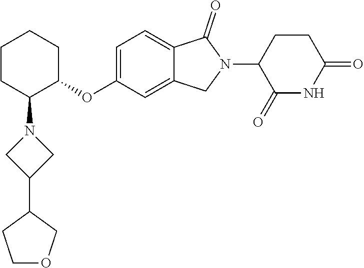 [SMILES: O=C1CCC(N2Cc3cc(O[C@H]4CCCC[C@@H]4N4CC(C5CCOC5)C4)ccc3C2=O)C(=O)N1] | 3-(1-oxo-5-(((1S,2S)-2-(3- (tetrahydrofuran-3-yl)azetidin-1- yl)cyclohexyl)oxy)isoindolin-2- yl)piperidine-2,6-dione |
| 15-A | 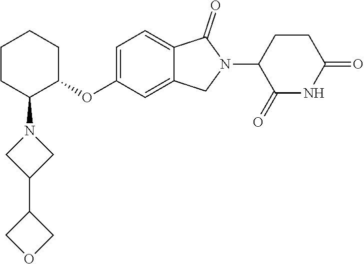 [SMILES: O=C1CCC(N2Cc3cc(O[C@H]4CCCC[C@@H]4N4CC(C5COC5)C4)ccc3C2=O)C(=O)N1] | 3-(5-(((1S,2S)-2-(3-(oxetan-3- yl)azetidin-1-yl)cyclohexyl)oxy)-1- oxoisoindolin-2-yl)piperidine-2,6- dione |
| 16-A | 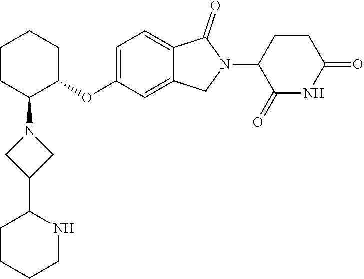 [SMILES: O=C1CCC(N2Cc3cc(O[C@H]4CCCC[C@@H]4N4CC(C5CCCCN5)C4)ccc3C2=O)C(=O)N1] | 3-(1-oxo-5-(((1S,2S)-2-(3- (piperidin-2-yl)azetidin-1- yl)cyclohexyl)oxy)isoindolin-2- yl)piperidine-2,6-dione |
| 17-A | 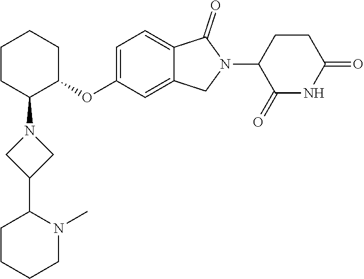 [SMILES: CN1CCCCC1C1CN([C@H]2CCCC[C@@H]2Oc2ccc3c(c2)CN(C2CCC(=O)NC2=O)C3=O)C1] | 3-(5-(((1S,2S)-2-(3-(1- methylpiperidin-2-yl)azetidin-1- yl)cyclohexyl)oxy)-1- oxoisoindolin-2-yl)piperidine-2,6- dione |
| 18-A | 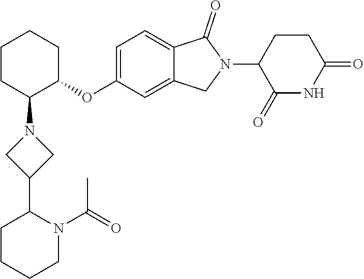 [SMILES: CC(=O)N1CCCCC1C1CN([C@H]2CCCC[C@@H]2Oc2ccc3c(c2)CN(C2CCC(=O)NC2=O)C3=O)C1] | 3-(5-(((1S,2S)-2-(3-(1- acetylpiperidin-2-yl)azetidin-1- yl)cyclohexyl)oxy)-1- oxoisoindolin-2-yl)piperidine-2,6- dione |
| 19-A | 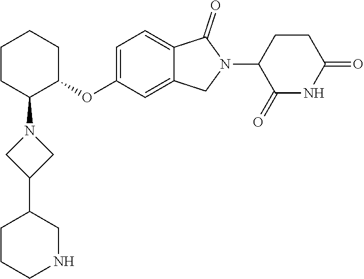 [SMILES: O=C1CCC(N2Cc3cc(O[C@H]4CCCC[C@@H]4N4CC(C5CCCNC5)C4)ccc3C2=O)C(=O)N1] | 3-(1-oxo-5-(((1S,2S)-2-(3- (piperidin-3-yl)azetidin-1- yl)cyclohexyl)oxy)isoindolin-2- yl)piperidine-2,6-dione |
| 20-A | 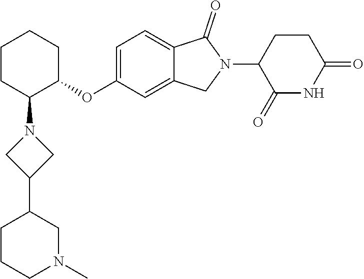 [SMILES: CN1CCCC(C2CN([C@H]3CCCC[C@@H]3Oc3ccc4c(c3)CN(C3CCC(=O)NC3=O)C4=O)C2)C1] | 3-(5-(((1,2S)-2-(3-(1- methylpiperidin-3-yl)azetidin-1- yl)cyclohexyl)oxy)-1- oxoisoindolin-2-yl)piperidine-2,6- dione |
| 21-A | 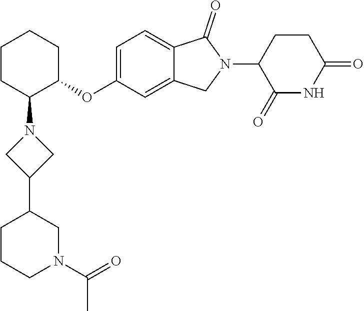 [SMILES: CC(=O)N1CCCC(C2CN([C@H]3CCCC[C@@H]3Oc3ccc4c(c3)CN(C3CCC(=O)NC3=O)C4=O)C2)C1] | 3-(5-(((1S,2S)-2-(3-(1- acetylpiperidin-3-yl)azetidin-1- yl)cyclohexyl)oxy)-1- oxoisoindolin-2-yl)piperidine-2,6- dione |
| 22-A | 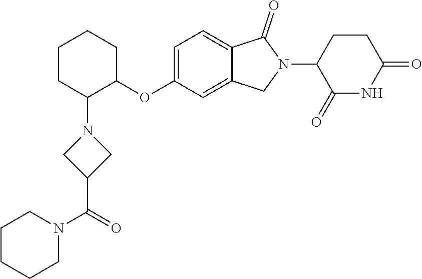 [SMILES: O=C1CCC(N2Cc3cc(OC4CCCCC4N4CC(C(=O)N5CCCCC5)C4)ccc3C2=O)C(=O)N1] | 3-(1-oxo-5-((2-(3-(piperidine-1- carbonyl)azetidin-1- yl)cyclohexyl)oxy)isoindolin-2- yl)piperidine-2,6-dione |
| | Second Eluting Isomer | |
| 23-A | 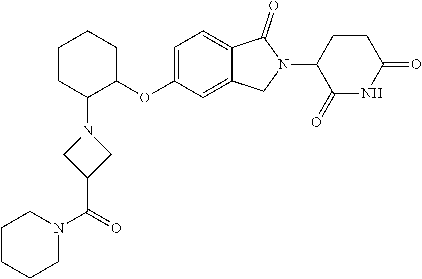 [SMILES: O=C1CCC(N2Cc3cc(OC4CCCCC4N4CC(C(=O)N5CCCCC5)C4)ccc3C2=O)C(=O)N1] | 3-(1-oxo-5-((2-(3-(piperidine-1- carbonyl)azetidin-1- yl)cyclohexyl)oxy)isoindolin-2- yl)piperidine-2,6-dione |
| | First Eluting Isomer | |
| 24-A | 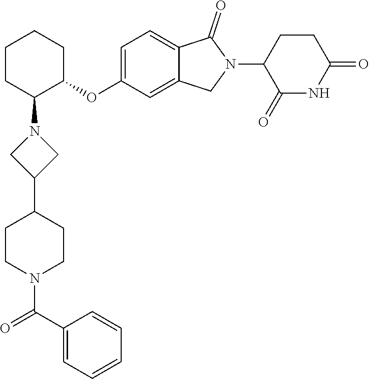 [SMILES: O=C1CCC(N2Cc3cc(O[C@H]4CCCC[C@@H]4N4CC(C5CCN(C(=O)c6ccccc6)CC5)C4)ccc3C2=O)C(=O)N1] | 3-(5-{[(1S,2S)-2-[3-(1- benzoylpiperidin-4-yl)azetidin-1- yl]cyclohexyl]oxy}-1-oxo-2,3- dihydro-1H-isoindol-2- yl)piperidine-2,6-dione |
| 25-A | 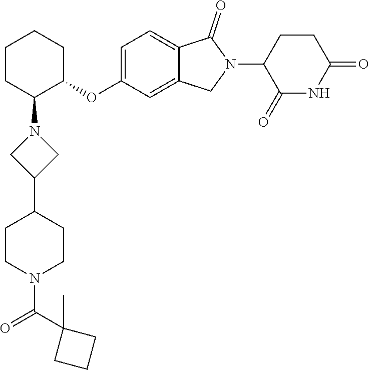 [SMILES: CC1(C(=O)N2CCC(C3CN([C@H]4CCCC[C@@H]4Oc4ccc5c(c4)CN(C4CCC(=O)NC4=O)C5=O)C3)CC2)CCC1] | 3-(5-{[(1S,2S)-2-{3-[1-(1- methylcyclobutanecarbonyl) piperidin-4-yl]azetidin-1- yl}cyclohexyl]oxy}-1-oxo-2,3- dihydro-1H-isoindol-2- yl)piperidine-2,6-dione |
| 26-A | 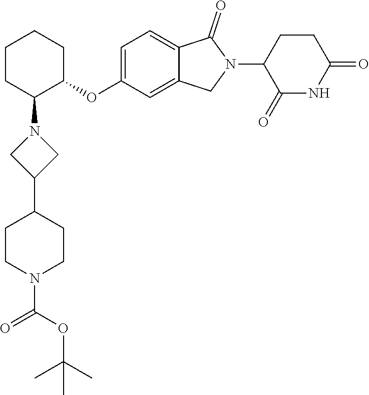 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1] | tert-butyl 4-{1-[(1S,2S)-2-{[2-(2,6- dioxopiperidin-3-yl)-1-oxo-2,3- dihydro-1H-isoindol-5- yl]oxy}cyclohexyl]azetidin-3- yl}piperidine-1-carboxylate |
| 27-A | 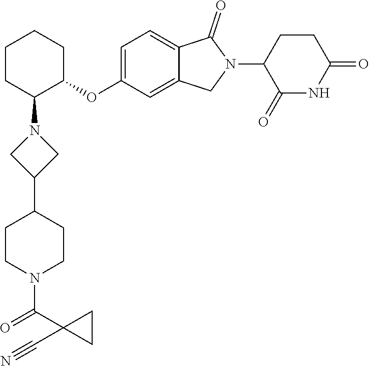 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1] | 1-(4-(1-((1S,2S)-2-((2-(2,6- dioxopiperidin-3-yl)-1- oxoisoindolin-5- yl)oxy)cyclohexyl)azetidin-3- yl)piperidine-1- carbonyl)cyclopropane-1- carbonitrile |
| 28-A | 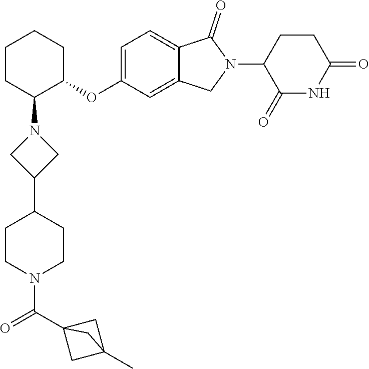 [SMILES: CC12CC(C(=O)N3CCC(C4CN([C@H]5CCCC[C@@H]5Oc5ccc6c(c5)CN(C5CCC(=O)NC5=O)C6=O)C4)CC3)(C1)C2] | 3-(5-(((1S,2S)-2-(3-(1-(3- methylbicyclo[1.1.1]pentane-1- carbonyl)piperidin-4-yl)azetidin-1- yl)cyclohexyl)oxy)-1- oxoisoindolin-2-yl)piperidine-2,6- dione |
| 29-A | 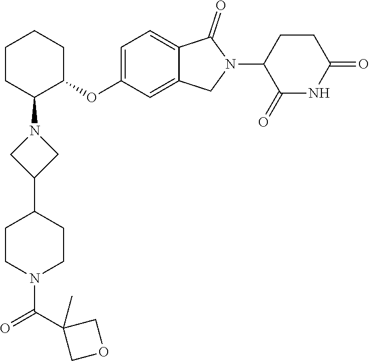 [SMILES: CC1(C(=O)N2CCC(C3CN([C@H]4CCCC[C@@H]4Oc4ccc5c(c4)CN(C4CCC(=O)NC4=O)C5=O)C3)CC2)COC1] | 3-(5-(((1S,2S)-2-(3-(1-(3- methyloxetane-3- carbonyl)piperidin-4-yl)azetidin-1- yl)cyclohexyl)oxy)-1- oxoisoindolin-2-yl)piperidine-2,6- dione |
| 30-A | 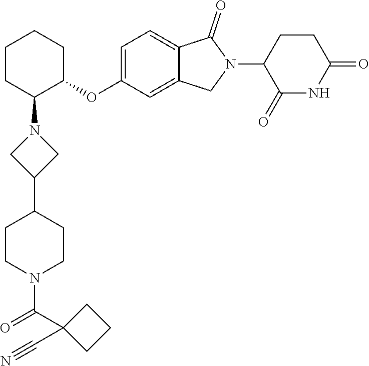 [SMILES: N#CC1(C(=O)N2CCC(C3CN([C@H]4CCCC[C@@H]4Oc4ccc5c(c4)CN(C4CCC(=O)NC4=O)C5=O)C3)CC2)CCC1] | 1-(4-(1-((1S,2S)-2-((2-(2,6- dioxopiperidin-3-yl)-1- oxoisoindolin-5- yl)oxy)cyclohexyl)azetidin-3- yl)piperidine-1- carbonyl)cyclobutane-1-carbonitrile |
| 31-A | 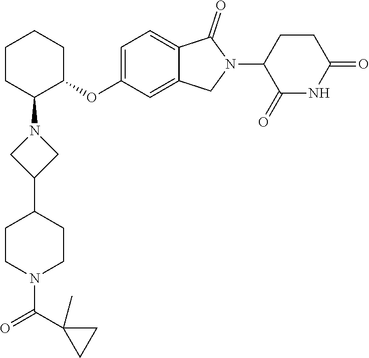 [SMILES: CC1(C(=O)N2CCC(C3CN([C@H]4CCCC[C@@H]4Oc4ccc5c(c4)CN(C4CCC(=O)NC4=O)C5=O)C3)CC2)CC1] | 3-(5-(((1S,2S)-2-(3-(1-(1- methylcyclopropane-1- carbonyl)piperidin-4-yl)azetidin-1- yl)cyclohexyl)oxy)-1- oxoisoindolin-2-yl)piperidine-2,6- dione |
| 32-A | 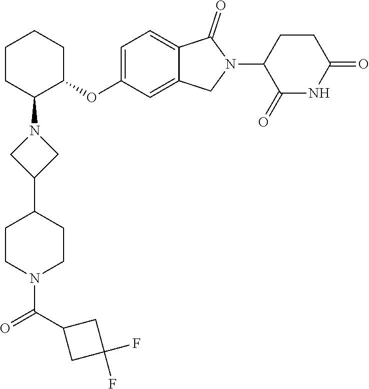 [SMILES: O=C1CCC(N2Cc3cc(O[C@H]4CCCC[C@@H]4N4CC(C5CCN(C(=O)C6CC(F)(F)C6)CC5)C4)ccc3C2=O)C(=O)N1] | 3-(5-(((1S,2S)-2-(3-(1-(3,3- difluorocyclobutane-1- carbonyl)piperidin-4-yl)azetidin-1- yl)cyclohexyl)oxy)-1- oxoisoindolin-2-yl)piperidine-2,6- dione |
| 33-A | 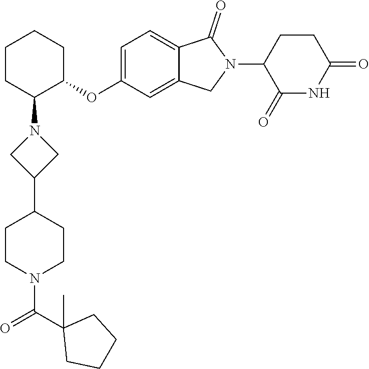 [SMILES: CC1(C(=O)N2CCC(C3CN([C@H]4CCCC[C@@H]4Oc4ccc5c(c4)CN(C4CCC(=O)NC4=O)C5=O)C3)CC2)CCCC1] | 3-(5-(((1S,2S)-2-(3-(1-(1- methylcyclopentane-1- carbonyl)piperidin-4-yl)azetidin-1- yl)cyclohexyl)oxy)-1- oxoisoindolin-2-yl)piperidine-2,6- dione |
| 34-A | 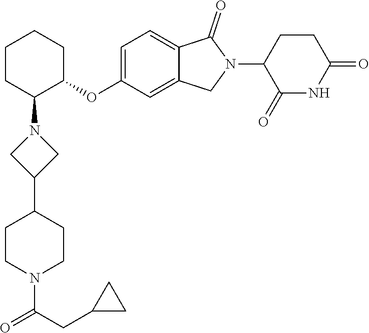 [SMILES: O=C1CCC(N2Cc3cc(O[C@H]4CCCC[C@@H]4N4CC(C5CCN(C(=O)CC6CC6)CC5)C4)ccc3C2=O)C(=O)N1] | 3-(5-(((1S,2S)-2-(3-(1-(2- cyclopropylacetyl)piperidin-4- yl)azetidin-1-yl)cyclohexyl)oxy)-1- oxoisoindolin-2-yl)piperidine-2,6- dione |
| 35-A | 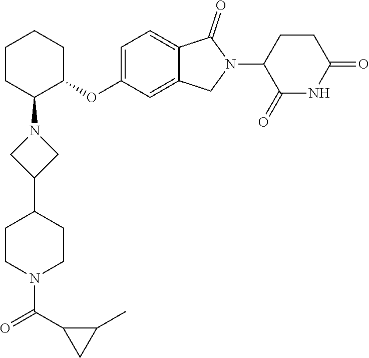 [SMILES: CC1CC1C(=O)N1CCC(C2CN([C@H]3CCCC[C@@H]3Oc3ccc4c(c3)CN(C3CCC(=O)NC3=O)C4=O)C2)CC1] | 3-(5-(((1S,2S)-2-(3-(1-(2- methylcyclopropane-1- carbonyl)piperidin-4-yl)azetidin-1- yl)cyclohexyl)oxy)-1- oxoisoindolin-2-yl)piperidine-2,6- dione |
| 36-A | 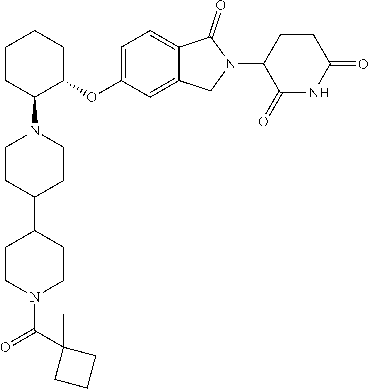 [SMILES: CC1(C(=O)N2CCC(C3CCN([C@H]4CCCC[C@@H]4Oc4ccc5c(c4)CN(C4CCC(=O)NC4=O)C5=O)CC3)CC2)CCC1] | 3-(5-(((1S,2S)-2-(1′-(1- methylcyclobutane-1-carbonyl)- [4,4′-bipiperidin]-1- yl)cyclohexyl)oxy)-1- oxoisoindolin-2-yl)piperidine-2,6- dione |
| 38-A | 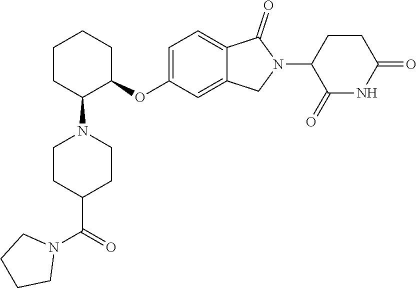 [SMILES: O=C1CCC(N2Cc3cc(O[C@@H]4CCCC[C@@H]4N4CCC(C(=O)N5CCCC5)CC4)ccc3C2=O)C(=O)N1] | 3-(1-oxo-5-(((1R,2S)-2-(4- (pyrrolidine-1-carbonyl)piperidin-1- yl)cyclohexyl)oxy)isoindolin-2- yl)piperidine-2,6-dione |
| 39-A | 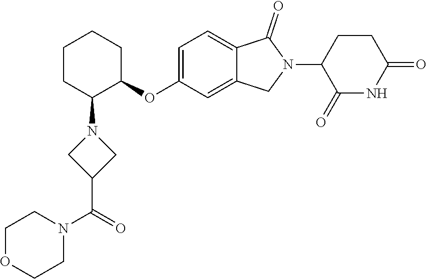 [SMILES: O=C1CCC(N2Cc3cc(O[C@@H]4CCCC[C@@H]4N4CC(C(=O)N5CCOCC5)C4)ccc3C2=O)C(=O)N1] | 3-(5-(((1R,2S)-2-(3-(morpholine-4- carbonyl)azetidin-1- yl)cyclohexyl)oxy)-1- oxoisoindolin-2-yl)piperidine-2,6- dione |
| 40-A | 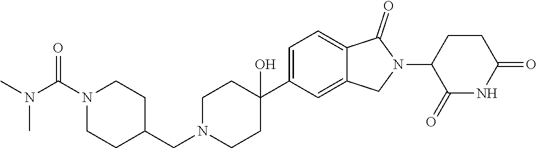 [SMILES: CN(C)C(=O)N1CCC(CN2CCC(O)(c3ccc4c(c3)CN(C3CCC(=O)NC3=O)C4=O)CC2)CC1] | 4-((4-(2-(2,6-dioxopiperidin-3-yl)- 1-oxoisoindolin-5-yl)-4- hydroxypiperidin-1-yl)methyl)- N,N-dimethylpiperidine-1- carboxamide |
| 41-A | 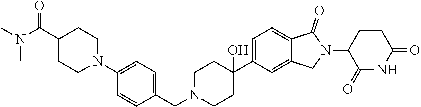 [SMILES: CN(C)C(=O)C1CCN(c2ccc(CN3CCC(O)(c4ccc5c(c4)CN(C4CCC(=O)NC4=O)C5=O)CC3)cc2)CC1] | 1-(4-((4-(2-(2,6-dioxopiperidin-3- yl)-1-oxoisoindolin-5-yl)-4- hydroxypiperidin-1- yl)methyl)phenyl)-N,N- dimethylpiperidine-4-carboxamide |
| 42-A | 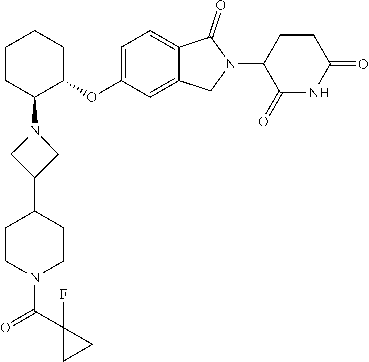 [SMILES: O=C1CCC(N2Cc3cc(O[C@H]4CCCC[C@@H]4N4CC(C5CCN(C(=O)C6(F)CC6)CC5)C4)ccc3C2=O)C(=O)N1] | 3-(5-(((1S,2S)-2-(3-(1-(1- fluorocyclopropane-1- carbonyl)piperidin-4-yl)azetidin-1- yl)cyclohexyl)oxy)-1- oxoisoindolin-2-yl)piperidine-2,6- dione |
| 43-A | 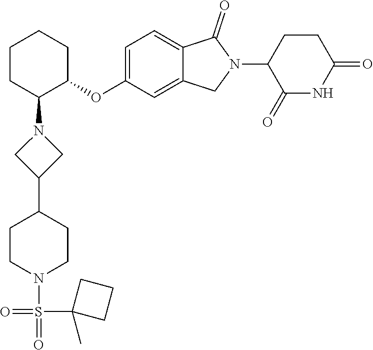 [SMILES: CC1(S(=O)(=O)N2CCC(C3CN([C@H]4CCCC[C@@H]4Oc4ccc5c(c4)CN(C4CCC(=O)NC4=O)C5=O)C3)CC2)CCC1] | 3-(5-(((1S,2S)-2-(3-(1-((1- methylcyclobutyl)sulfonyl) piperidin-4-yl)azetidin-1- yl)cyclohexyl)oxy)-1- oxoisoindolin-2-yl)piperidine-2,6- dione |
| 44-A | 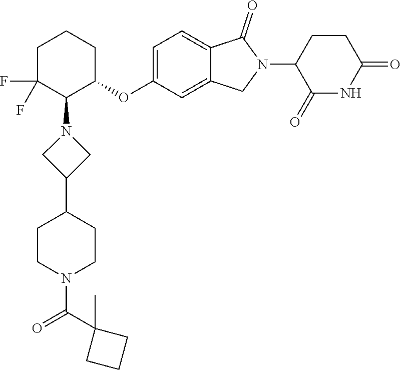 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCC1] | 3-(5-(((1S,2R)-3,3-difluoro-2-(3-(1- (1-methylcyclobutane-1- carbonyl)piperidin-4-yl)azetidin-1- yl)cyclohexyl)oxy)-1- oxoisoindolin-2-yl)piperidine-2,6- dione |
| 45-A | 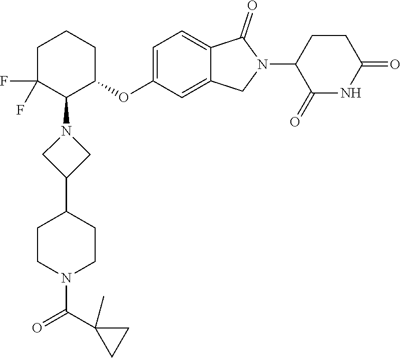 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CC1] | 3-(5-(((1S,2R)-3,3-difluoro-2-(3-(1- (1-methylcyclopropane-1- carbonyl)piperidin-4-yl)azetidin-1- yl)cyclohexyl)oxy)-1- oxoisoindolin-2-yl)piperidine-2,6- dione |
| 46-A | 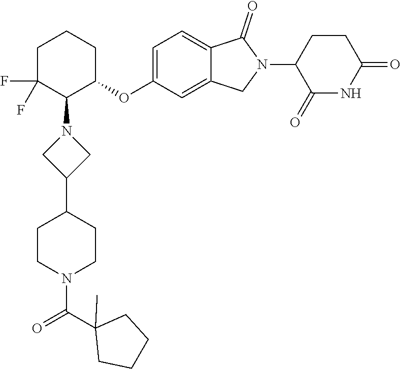 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCCC1] | 3-(5-(((1S,2R)-3,3-difluoro-2-(3-(1- (1-methylcyclopentane-1- carbonyl)piperidin-4-yl)azetidin-1- yl)cyclohexyl)oxy)-1- oxoisoindolin-2-yl)piperidine-2,6- dione |
| 47-A | 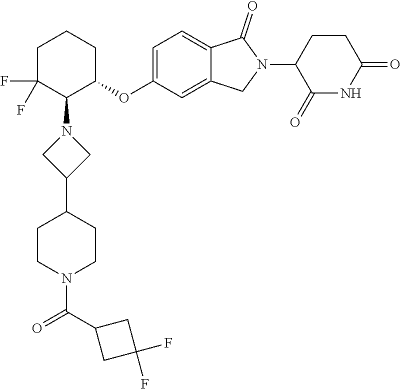 [SMILES: O=C1CCC(N2Cc3cc(O[C@H]4CCCC(F)(F)[C@@H]4N4CC(C5CCN(C(=O)C6CC(F)(F)C6)CC5)C4)ccc3C2=O)C(=O)N1] | 3-(5-(((1S,2R)-2-(3-(1-(3,3- difluorocyclobutane-1- carbonyl)piperidin-4-yl)azetidin-1- yl)-3,3-difluorocyclohexyl)oxy)-1- oxoisoindolin-2-yl)piperidine-2,6- dione |
| 48-A | 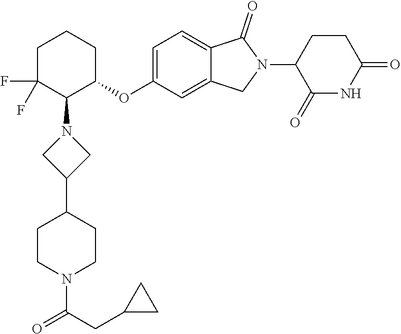 [SMILES: O=C1CCC(N2Cc3cc(O[C@H]4CCCC(F)(F)[C@@H]4N4CC(C5CCN(C(=O)CC6CC6)CC5)C4)ccc3C2=O)C(=O)N1] | 3-(5-(((1S,2R)-2-(3-(1-(2- cyclopropylacetyl)piperidin-4- yl)azetidin-1-yl)-3,3- difluorocyclohexyl)oxy)-1- oxoisoindolin-2-yl)piperidine-2,6- dione |
| 49-A | 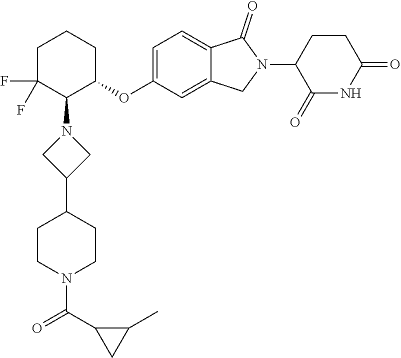 [SMILES: CC1CC1C(=O)N1CCC(C2CN([C@@H]3[C@@H](Oc4ccc5c(c4)CN(C4CCC(=O)NC4=O)C5=O)CCCC3(F)F)C2)CC1] | 3-(5-(((1S,2R)-3,3-difluoro-2-(3-(1- (2-methylcyclopropane-1- carbonyl)piperidin-4-yl)azetidin-1- yl)cyclohexyl)oxy)-1- oxoisoindolin-2-yl)piperidine-2,6- dione |
| 50-A | 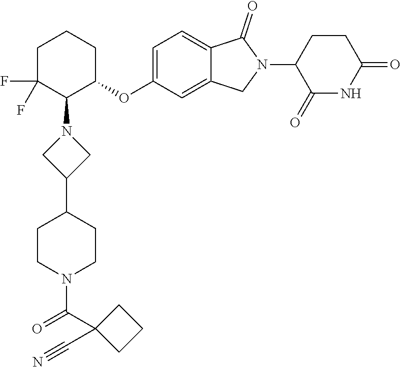 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CCC1] | 1-(4-(1-((1R,6S)-6-((2-(2,6- dioxopiperidin-3-yl)-1- oxoisoindolin-5-yl)oxy)-2,2- difluorocyclohexyl)azetidin-3- yl)piperidine-1- carbonyl)cyclobutane-1-carbonitrile |
| 51-A | 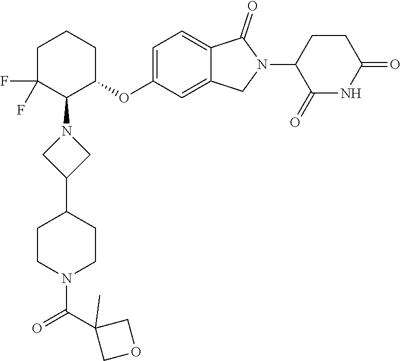 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)COC1] | 3-(5-(((1S,2R)-3,3-difluoro-2-(3-(1- (3-methyloxetane-3- carbonyl)piperidin-4-yl)azetidin-1- yl)cyclohexyl)oxy)-1- oxoisoindolin-2-yl)piperidine-2,6- dione |
| 52-A | 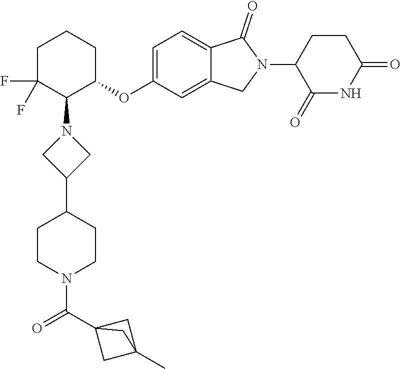 [SMILES: CC12CC(C(=O)N3CCC(C4CN([C@@H]5[C@@H](Oc6ccc7c(c6)CN(C6CCC(=O)NC6=O)C7=O)CCCC5(F)F)C4)CC3)(C1)C2] | 3-(5-(((1S,2R)-3,3-difluoro-2-(3-(1- (3-methylbicyclo[1.1.1]pentane-1- carbonyl)piperidin-4-yl)azetidin-1- yl)cyclohexyl)oxy)-1- oxoisoindolin-2-yl)piperidine-2,6- dione |
| 53-A | 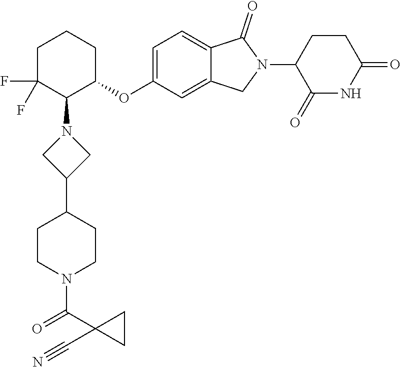 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CC1] | 1-(4-(1-((1R,6S)-6-((2-(2,6- dioxopiperidin-3-yl)-1- oxoisoindolin-5-yl)oxy)-2,2- difluorocyclohexyl)azetidin-3- yl)piperidine-1- carbonyl)cyclopropane-1- carbonitrile |
| 54-A | 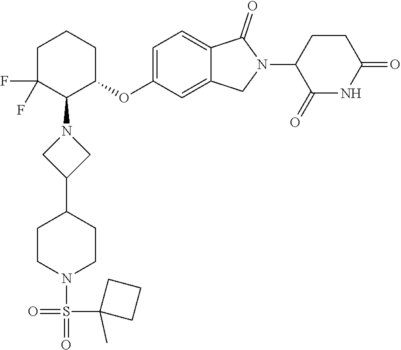 [SMILES: CC1(S(=O)(=O)N2CCC(C3CN([C@@H]4[C@@H](Oc5ccc6c(c5)CN(C5CCC(=O)NC5=O)C6=O)CCCC4(F)F)C3)CC2)CCC1] | 3-(5-(((1S,2R)-3,3-difluoro-2-(3-(1- ((1- methylcyclobutyl)sulfonyl)piperidin- 4-yl)azetidin-1- yl)cyclohexyl)oxy)-1- oxoisoindolin-2-yl)piperidine-2,6- dione |
| 55-A | 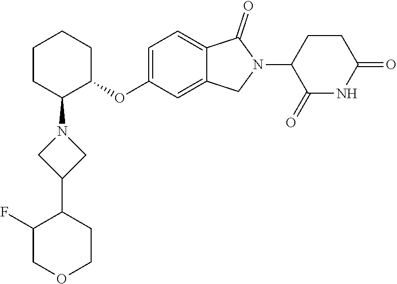 [SMILES: O=C1CCC(N2Cc3cc(O[C@H]4CCCC[C@@H]4N4CC(C5CCOCC5F)C4)ccc3C2=O)C(=O)N1] | 3-(5-(((1S,2S)-2-(3-(3- fluorotetrahydro-2H-pyran-4- yl)azetidin-1-yl)cyclohexyl)oxy)-1- oxoisoindolin-2-yl)piperidine-2,6- dione |
| 56-A | 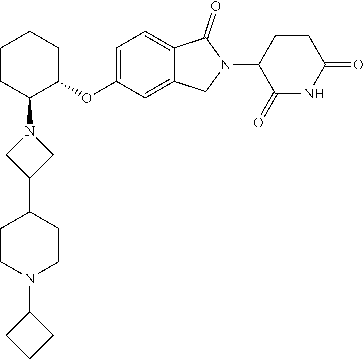 [SMILES: O=C1CCC(N2Cc3cc(O[C@H]4CCCC[C@@H]4N4CC(C5CCN(C6CCC6)CC5)C4)ccc3C2=O)C(=O)N1] | 3-(5-(((1S,2S)-2-(3-(1- cyclobutylpiperidin-4-yl)azetidin-1- yl)cyclohexyl)oxy)-1- oxoisoindolin-2-yl)piperidine-2,6- dione |
| 57-A | 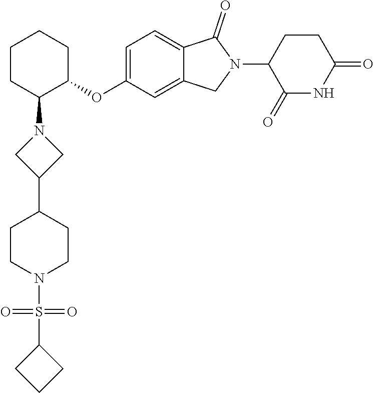 [SMILES: O=C1CCC(N2Cc3cc(O[C@H]4CCCC[C@@H]4N4CC(C5CCN(S(=O)(=O)C6CCC6)CC5)C4)ccc3C2=O)C(=O)N1] | 3-(5-(((1S,2S)-2-(3-(1- (cyclobutylsulfonyl)piperidin-4- yl)azetidin-1-yl)cyclohexyl)oxy)-1- oxoisoindolin-2-yl)piperidine-2,6- dione |
| 58-A | 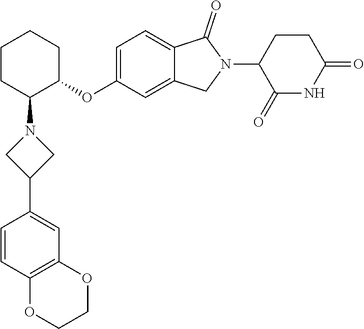 [SMILES: O=C1CCC(N2Cc3cc(O[C@H]4CCCC[C@@H]4N4CC(c5ccc6c(c5)OCCO6)C4)ccc3C2=O)C(=O)N1] | 3-(5-(((1S,2S)-2-(3-(2,3- dihydrobenzo[b][1,4]dioxin-6- yl)azetidin-1-yl)cyclohexyl)oxy)-1- oxoisoindolin-2-yl)piperidine-2,6- dione |
| 1-B | 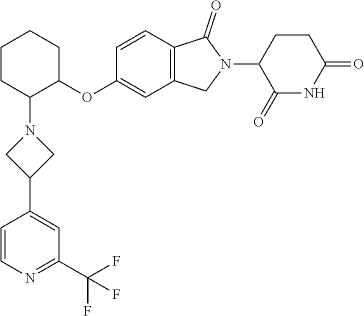 [SMILES: O=C1CCC(N2Cc3cc(OC4CCCCC4N4CC(c5ccnc(C(F)(F)F)c5)C4)ccc3C2=O)C(=O)N1] | 3-(1-oxo-5-((2-(3-(2- (trifluoromethyl)-pyridin-4- yl)azetidin-1-yl)cyclohexyl)- oxy)isoindolin-2-yl)piperidine-2,6- dione |
| 2-B | 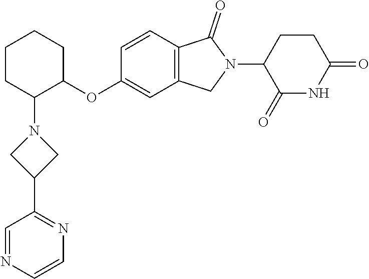 [SMILES: O=C1CCC(N2Cc3cc(OC4CCCCC4N4CC(c5cnccn5)C4)ccc3C2=O)C(=O)N1] | 3-(1-oxo-5-((2-(3-(pyrazin-2- yl)azetidin-1- yl)cyclohexyl)oxy)isoindolin-2- yl)piperidine-2,6-dione |
| 3-B | 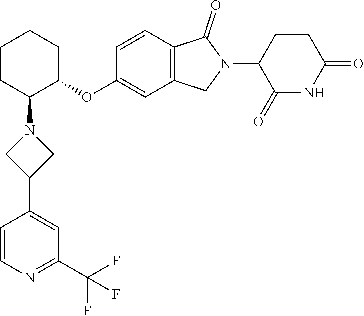 [SMILES: O=C1CCC(N2Cc3cc(O[C@H]4CCCC[C@@H]4N4CC(c5ccnc(C(F)(F)F)c5)C4)ccc3C2=O)C(=O)N1] | 3-(1-oxo-5-(((1S,2S)-2-(3-(2- (trifluoromethyl)pyridin-4- yl)azetidin-1- yl)cyclohexyl)oxy)isoindolin-2- yl)piperidine-2,6-dione |
| 4-B | 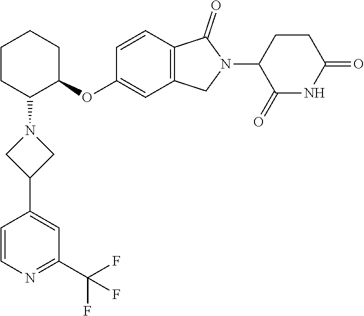 | 3-(1-oxo-5-(((1R,2R)-2-(3-(2- (trifluoromethyl)pyridin-4- yl)azetidin-1- yl)cyclohexyl)oxy)isoindolin-2- yl)piperidine-2,6-dione |
| 5-B | 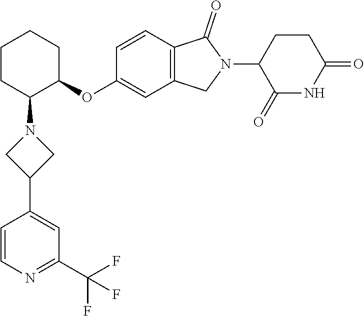 [SMILES: O=C1CCC(N2Cc3cc(O[C@@H]4CCCC[C@@H]4N4CC(c5ccnc(C(F)(F)F)c5)C4)ccc3C2=O)C(=O)N1] | 3-(1-oxo-5-(((1R,2S)-2-(3-(2- (trifluoromethyl)pyridin-4- yl)azetidin-1- yl)cyclohexyl)oxy)isoindolin-2- yl)piperidine-2,6-dione |
| 6-B | 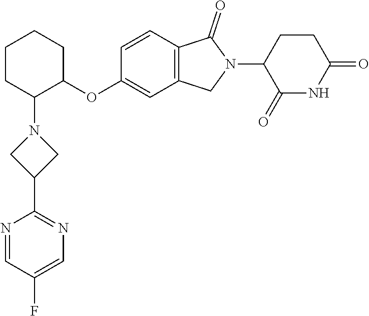 [SMILES: O=C1CCC(N2Cc3cc(OC4CCCCC4N4CC(c5ncc(F)cn5)C4)ccc3C2=O)C(=O)N1] | 3-(5-((2-(3-(5-fluoropyrimidin-2- yl)azetidin-1-yl)cyclohexyl)-oxy)- 1-oxoisoindolin-2-yl)piperidine- 2,6-dione |
| 7-B | 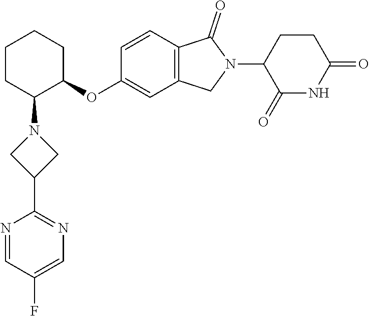 [SMILES: O=C1CCC(N2Cc3cc(O[C@@H]4CCCC[C@@H]4N4CC(c5ncc(F)cn5)C4)ccc3C2=O)C(=O)N1] | 3-(5-(((1R,2S)-2-(3-(5- fluoropyrimidin-2-yl)azetidin-1- yl)cyclohexyl)oxy)-1- oxoisoindolin-2-yl)piperidine-2,6- dione |
| 8-B | 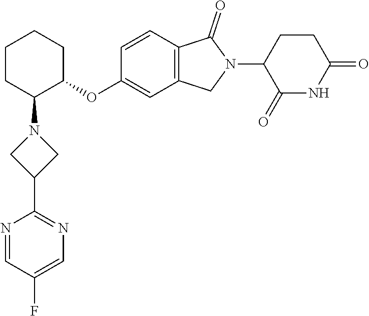 [SMILES: O=C1CCC(N2Cc3cc(O[C@H]4CCCC[C@@H]4N4CC(c5ncc(F)cn5)C4)ccc3C2=O)C(=O)N1] | 3-(5-(((1S,2S)-2-(3-(5- fluoropyrimidin-2-yl)azetidin-1- yl)cyclohexyl)oxy)-1- oxoisoindolin-2-yl)piperidine-2,6- dione |
| 9-B | 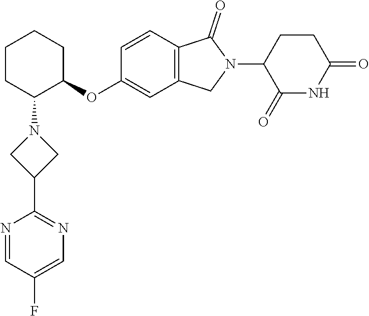 [SMILES: O=C1CCC(N2Cc3cc(O[C@@H]4CCCC[C@H]4N4CC(c5ncc(F)cn5)C4)ccc3C2=O)C(=O)N1] | 3-(5-(((1R,2R)-2-(3-(5- fluoropyrimidin-2-yl)azetidin-1- yl)cyclohexyl)oxy)-1- oxoisoindolin-2-yl)piperidine-2,6- dione |
| 10-B | 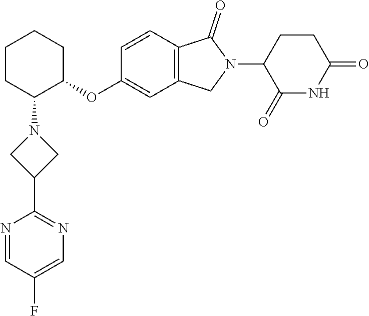 [SMILES: O=C1CCC(N2Cc3cc(O[C@H]4CCCC[C@H]4N4CC(c5ncc(F)cn5)C4)ccc3C2=O)C(=O)N1] | 3-(5-(((1S,2R)-2-(3-(5- fluoropyrimidin-2-yl)azetidin-1- yl)cyclohexyl)oxy)-1- oxoisoindolin-2-yl)piperidine-2,6- dione |
| 11-B | 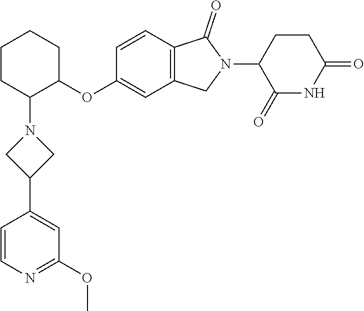 [SMILES: COc1cc(C2CN(C3CCCCC3Oc3ccc4c(c3)CN(C3CCC(=O)NC3=O)C4=O)C2)ccn1] | 3-(5-((2-(3-(2-methoxypyridin-4- yl)azetidin-1-yl)cyclohexyl)oxy)-1- oxoisoindolin-2-yl)piperidine-2,6- dione |
| 12-B | 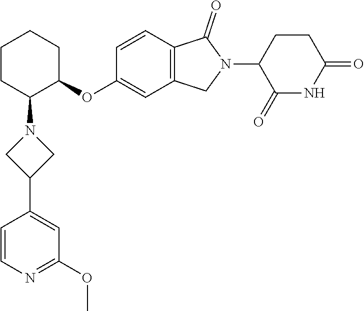 [SMILES: COc1cc(C2CN([C@H]3CCCC[C@H]3Oc3ccc4c(c3)CN(C3CCC(=O)NC3=O)C4=O)C2)ccn1] | 3-(5-(((1R,2S)-2-(3-(2- methoxypyridin-4-yl)azetidin-1- yl)cyclohexyl)oxy)-1- oxoisoindolin-2-yl)piperidine-2,6- dione |
| 13-B | 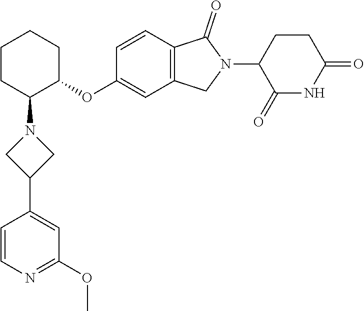 [SMILES: COc1cc(C2CN([C@H]3CCCC[C@@H]3Oc3ccc4c(c3)CN(C3CCC(=O)NC3=O)C4=O)C2)ccn1] | 3-(5-(((1S,2S)-2-(3-(2- methoxypyridin-4-yl)azetidin-1- yl)cyclohexyl)oxy)-1- oxoisoindolin-2-yl)piperidine-2,6- dione |
| 14-B | 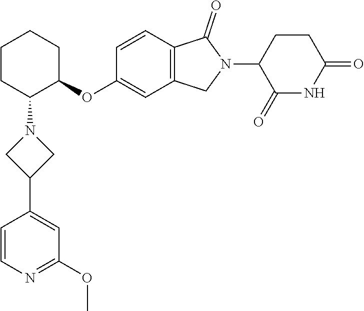 [SMILES: COc1cc(C2CN([C@@H]3CCCC[C@H]3Oc3ccc4c(c3)CN(C3CCC(=O)NC3=O)C4=O)C2)ccn1] | 3-(5-(((1R,2R)-2-(3-(2- methoxypyridin-4-yl)azetidin-1- yl)cyclohexyl)oxy)-1- oxoisoindolin-2-yl)piperidine-2,6- dione |
| 15-B | 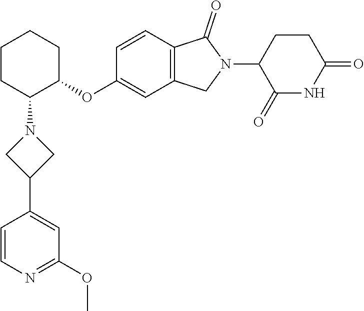 [SMILES: COc1cc(C2CN([C@@H]3CCCC[C@@H]3Oc3ccc4c(c3)CN(C3CCC(=O)NC3=O)C4=O)C2)ccn1] | 3-(5-(((1S,2R)-2-(3-(2- methoxypyridin-4-yl)azetidin-1- yl)cyclohexyl)oxy)-1- oxoisoindolin-2-yl)piperidine-2,6- dione |
| 16-B | 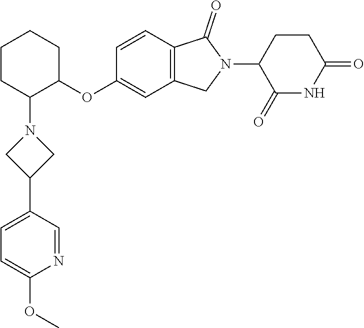 [SMILES: COc1ccc(C2CN(C3CCCCC3Oc3ccc4c(c3)CN(C3CCC(=O)NC3=O)C4=O)C2)cn1] | 3-(5-((2-(3-(6-methoxypyridin-3- yl)azetidin-1-yl)cyclohexyl)oxy)-1- oxoisoindolin-2-yl)piperidine-2,6- dione |
| 17-B | 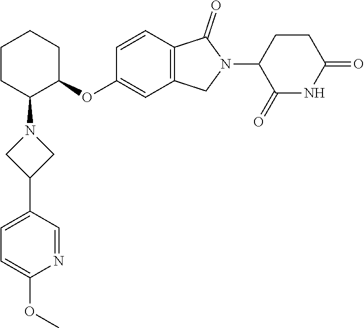 [SMILES: COc1ccc(C2CN([C@H]3CCCC[C@H]3Oc3ccc4c(c3)CN(C3CCC(=O)NC3=O)C4=O)C2)cn1] | 3-(5-(((1R,2S)-2-(3-(6- methoxypyridin-3-yl)azetidin-1- yl)cyclohexyl)oxy)-1- oxoisoindolin-2-yl)piperidine-2,6- dione |
| 18-B | 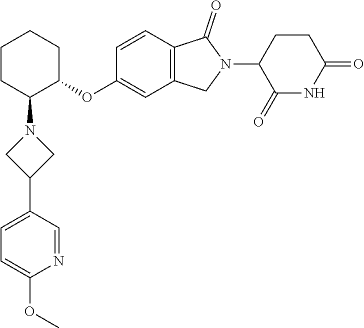 [SMILES: COc1ccc(C2CN([C@H]3CCCC[C@@H]3Oc3ccc4c(c3)CN(C3CCC(=O)NC3=O)C4=O)C2)cn1] | 3-(5-(((1S,2S)-2-(3-(6- methoxypyridin-3-yl)azetidin-1- yl)cyclohexyl)oxy)-1- oxoisoindolin-2-yl)piperidine-2,6- dione |
| 19-B | 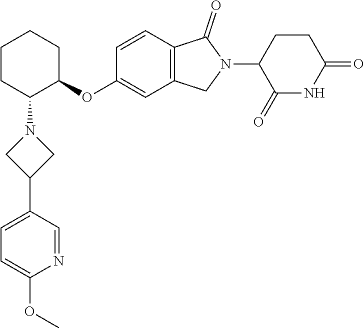 [SMILES: COc1ccc(C2CN([C@@H]3CCCC[C@H]3Oc3ccc4c(c3)CN(C3CCC(=O)NC3=O)C4=O)C2)cn1] | 3-(5-(((1R,2R)-2-(3-(6- methoxypyridin-3-yl)azetidin-1- yl)cyclohexyl)oxy)-1- oxoisoindolin-2-yl)piperidine-2,6- dione |
| 20-B | 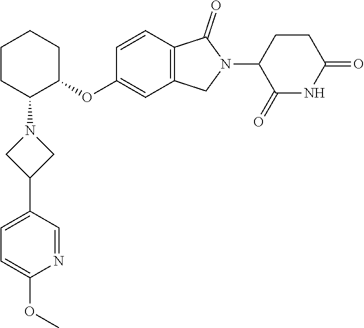 [SMILES: COc1ccc(C2CN([C@@H]3CCCC[C@@H]3Oc3ccc4c(c3)CN(C3CCC(=O)NC3=O)C4=O)C2)cn1] | 3-(5-(((1S,2R)-2-(3-(6- methoxypyridin-3-yl)azetidin-1- yl)cyclohexyl)oxy)-1- oxoisoindolin-2-yl)piperidine-2,6- dione |
| 21-B | 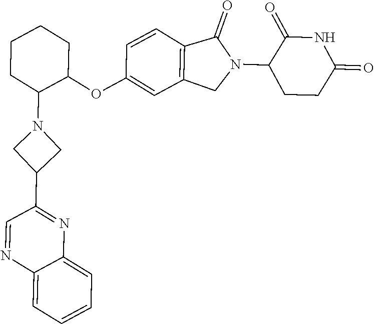 [SMILES: O=C1CCC(N2Cc3cc(OC4CCCCC4N4CC(c5cnc6ccccc6n5)C4)ccc3C2=O)C(=O)N1] | 3-(1-oxo-5-((2-(3-(quinoxalin-2- yl)azetidin-1- yl)cyclohexyl)oxy)isoindolin-2- yl)piperidine-2,6-dione |
| 22-B | 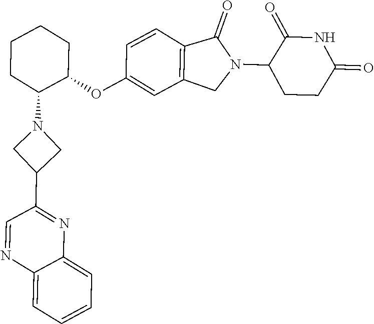 [SMILES: O=C1CCC(N2Cc3cc(O[C@H]4CCCC[C@H]4N4CC(c5cnc6ccccc6n5)C4)ccc3C2=O)C(=O)N1] | 3-(1-oxo-5-(((1S,2R)-2-(3- (quinoxalin-2-yl)azetidin-1- yl)cyclohexyl)oxy)isoindolin-2- yl)piperidine-2,6-dione |
| 23-B | 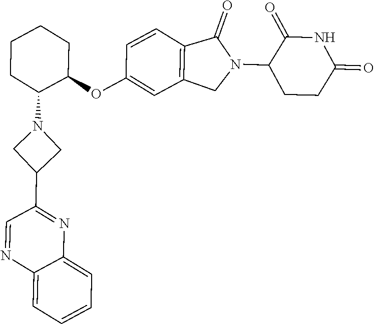 [SMILES: O=C1CCC(N2Cc3cc(O[C@@H]4CCCC[C@H]4N4CC(c5cnc6ccccc6n5)C4)ccc3C2=O)C(=O)N1] | 3-(1-oxo-5-(((1R,2R)-2-(3- (quinoxalin-2-yl)azetidin-1- yl)cyclohexyl)oxy)isoindolin-2- yl)piperidine-2,6-dione |
| 24-B | 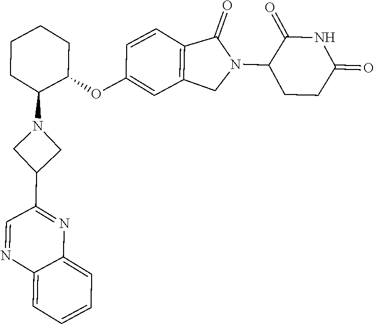 [SMILES: O=C1CCC(N2Cc3cc(O[C@H]4CCCC[C@@H]4N4CC(c5cnc6ccccc6n5)C4)ccc3C2=O)C(=O)N1] | 3-(1-oxo-5-(((1S,2S)-2-(3- (quinoxalin-2-yl)azetidin-1- yl)cyclohexyl)oxy)isoindolin-2- yl)piperidine-2,6-dione |
| 25-B | 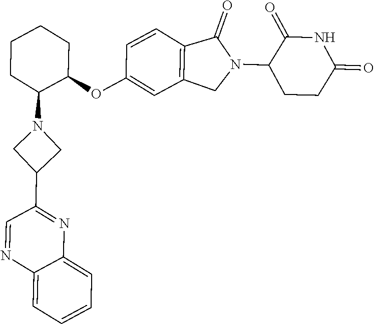 [SMILES: O=C1CCC(N2Cc3cc(O[C@@H]4CCCC[C@@H]4N4CC(c5cnc6ccccc6n5)C4)ccc3C2=O)C(=O)N1] | 3-(1-oxo-5-(((1R,2S)-2-(3- (quinoxalin-2-yl)azetidin-1- yl)cyclohexyl)oxy)isoindolin-2- yl)piperidine-2,6-dione |
| 26-B | 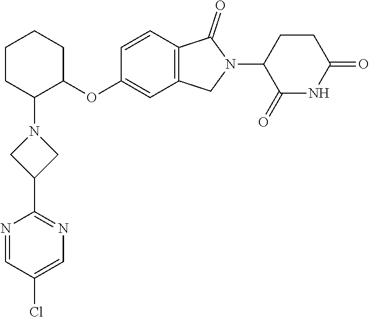 [SMILES: O=C1CCC(N2Cc3cc(OC4CCCCC4N4CC(c5ncc(Cl)cn5)C4)ccc3C2=O)C(=O)N1] | 3-(5-((2-(3-(5-chloropyrimidin-2- yl)azetidin-1-yl)cyclohexyl)oxy)-1- oxoisoindolin-2-yl)piperidine-2,6- dione |
| 27-B | 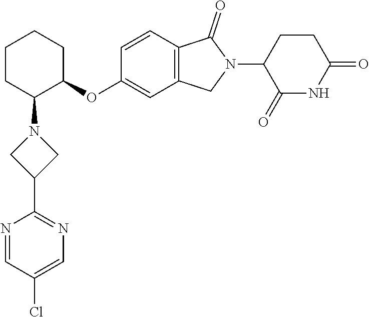 [SMILES: O=C1CCC(N2Cc3cc(O[C@@H]4CCCC[C@@H]4N4CC(c5ncc(Cl)cn5)C4)ccc3C2=O)C(=O)N1] | 3-(5-(((1R,2S)-2-(3-(5- chloropyrimidin-2-yl)azetidin-1- yl)cyclohexyl)oxy)-1- oxoisoindolin-2-yl)piperidine-2,6- dione |
| 28-B | 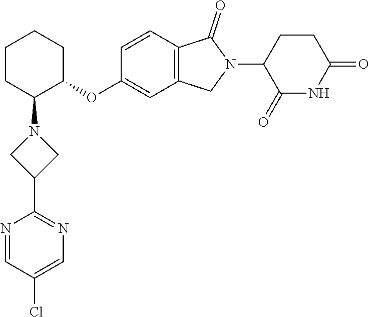 [SMILES: O=C1CCC(N2Cc3cc(O[C@H]4CCCC[C@@H]4N4CC(c5ncc(Cl)cn5)C4)ccc3C2=O)C(=O)N1] | 3-(5-(((1S,2S)-2-(3-(5- chloropyrimidin-2-yl)azetidin-1- yl)cyclohexyl)oxy)-1- oxoisoindolin-2-yl)piperidine-2,6- dione |
| 29-B | 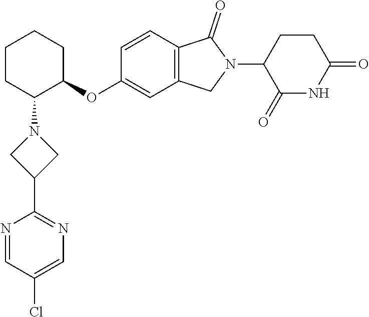 [SMILES: O=C1CCC(N2Cc3cc(O[C@@H]4CCCC[C@H]4N4CC(c5ncc(Cl)cn5)C4)ccc3C2=O)C(=O)N1] | 3-(5-(((1R,2R)-2-(3-(5- chloropyrimidin-2-yl)azetidin-1- yl)cyclohexyl)oxy)-1- oxoisoindolin-2-yl)piperidine-2,6- dione |
| 30-B | 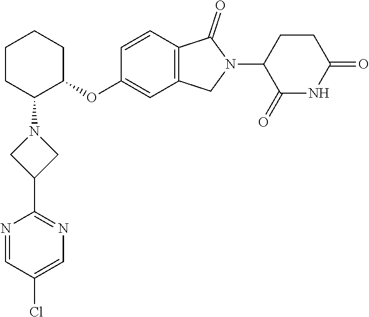 [SMILES: O=C1CCC(N2Cc3cc(O[C@H]4CCCC[C@H]4N4CC(c5ncc(Cl)cn5)C4)ccc3C2=O)C(=O)N1] | 3-(5-(((1S,2R)-2-(3-(5- chloropyrimidin-2-yl)azetidin-1- yl)cyclohexyl)oxy)-1- oxoisoindolin-2-yl)piperidine-2,6- dione |
| 31-B | 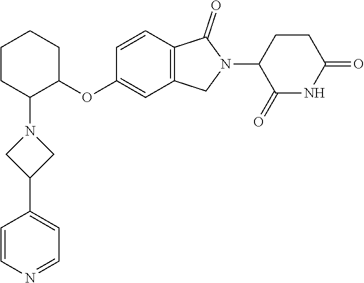 [SMILES: O=C1CCC(N2Cc3cc(OC4CCCCC4N4CC(c5ccncc5)C4)ccc3C2=O)C(=O)N1] | 3-(1-oxo-5-((2-(3-(pyridin-4- yl)azetidin-1- yl)cyclohexyl)oxy)isoindolin-2- yl)piperidine-2,6-dione |
| 32-B | 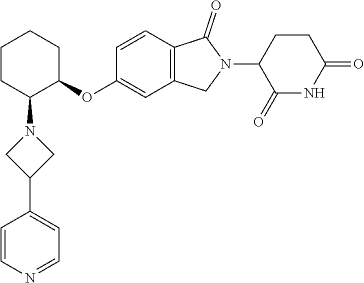 [SMILES: O=C1CCC(N2Cc3cc(O[C@@H]4CCCC[C@@H]4N4CC(c5ccncc5)C4)ccc3C2=O)C(=O)N1] | 3-(1-oxo-5-(((1R,2S)-2-(3-(pyridin- 4-yl)azetidin-1- yl)cyclohexyl)oxy)isoindolin-2- yl)piperidine-2,6-dione |
| 33-B | 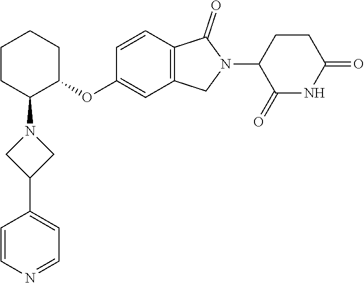 [SMILES: O=C1CCC(N2Cc3cc(O[C@H]4CCCC[C@@H]4N4CC(c5ccncc5)C4)ccc3C2=O)C(=O)N1] | 3-(1-oxo-5-(((1S,2S)-2-(3-(pyridin- 4-yl)azetidin-1- yl)cyclohexyl)oxy)isoindolin-2- yl)piperidine-2,6-dione |
| 34-B | 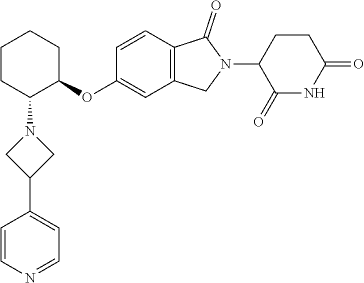 [SMILES: O=C1CCC(N2Cc3cc(O[C@@H]4CCCC[C@H]4N4CC(c5ccncc5)C4)ccc3C2=O)C(=O)N1] | 3-(1-oxo-5-(((1R,2R)-2-(3-(pyridin- 4-yl)azetidin-1- yl)cyclohexyl)oxy)isoindolin-2- yl)piperidine-2,6-dione |
| 35-B | 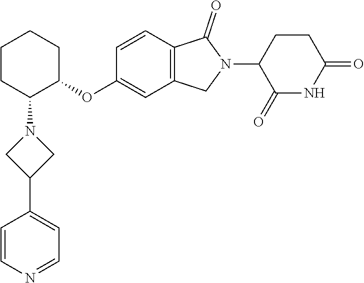 [SMILES: O=C1CCC(N2Cc3cc(O[C@H]4CCCC[C@H]4N4CC(c5ccncc5)C4)ccc3C2=O)C(=O)N1] | 3-(1-oxo-5-(((1S,2R)-2-(3-(pyridin- 4-yl)azetidin-1- yl)cyclohexyl)oxy)isoindolin-2- yl)piperidine-2,6-dione |
| 36-B | 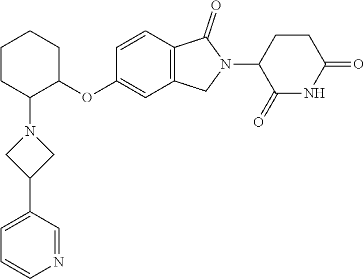 [SMILES: O=C1CCC(N2Cc3cc(OC4CCCCC4N4CC(c5cccnc5)C4)ccc3C2=O)C(=O)N1] | 3-(1-oxo-5-((2-(3-(pyridin-3- yl)azetidin-1- yl)cyclohexyl)oxy)isoindolin-2- yl)piperidine-2,6-dione |
| 37-B | 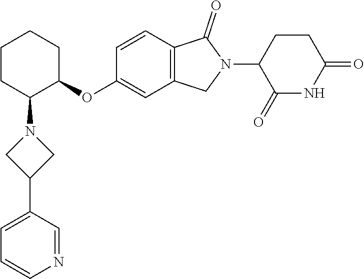 [SMILES: O=C1CCC(N2Cc3cc(O[C@@H]4CCCC[C@@H]4N4CC(c5cccnc5)C4)ccc3C2=O)C(=O)N1] | 3-(1-oxo-5-(((1R,2S)-2-(3-(pyridin- 3-yl)azetidin-1- yl)cyclohexyl)oxy)isoindolin-2- yl)piperidine-2,6-dione |
| 38-B | 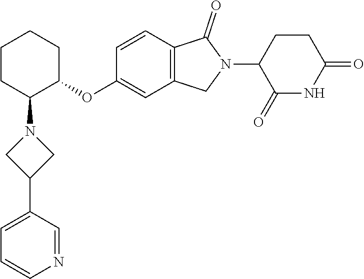 [SMILES: O=C1CCC(N2Cc3cc(O[C@H]4CCCC[C@@H]4N4CC(c5cccnc5)C4)ccc3C2=O)C(=O)N1] | 3-(1-oxo-5-(((1S,2S)-2-(3-(pyridin- 3-yl)azetidin-1- yl)cyclohexyl)oxy)isoindolin-2- yl)piperidine-2,6-dione |
| 39-B | 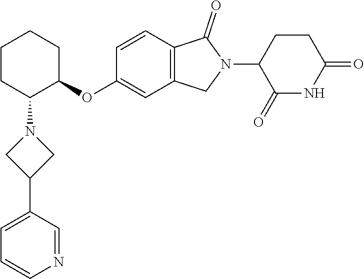 [SMILES: O=C1CCC(N2Cc3cc(O[C@@H]4CCCC[C@H]4N4CC(c5cccnc5)C4)ccc3C2=O)C(=O)N1] | 3-(1-oxo-5-(((1R,2R)-2-(3-(pyridin- 3-yl)azetidin-1- yl)cyclohexyl)oxy)isoindolin-2- yl)piperidine-2,6-dione |
| 40-B | 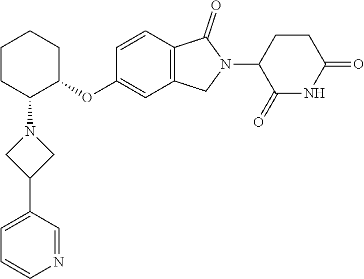 [SMILES: O=C1CCC(N2Cc3cc(O[C@H]4CCCC[C@H]4N4CC(c5cccnc5)C4)ccc3C2=O)C(=O)N1] | 3-(1-oxo-5-(((1S,2R)-2-(3-(pyridin- 3-yl)azetidin-1- yl)cyclohexyl)oxy)isoindolin-2- yl)piperidine-2,6-dione |
| 41-B | 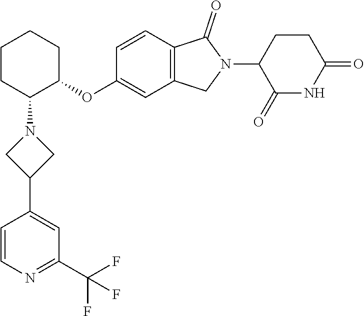 [SMILES: O=C1CCC(N2Cc3cc(O[C@H]4CCCC[C@H]4N4CC(c5ccnc(C(F)(F)F)c5)C4)ccc3C2=O)C(=O)N1] | 3-(1-oxo-5-(((1S,2R)-2-(3-(2- (trifluoromethyl)pyridin-4- yl)azetidin-1- yl)cyclohexyl)oxy)isoindolin-2- yl)piperidine-2,6-dione |
| 42-B | 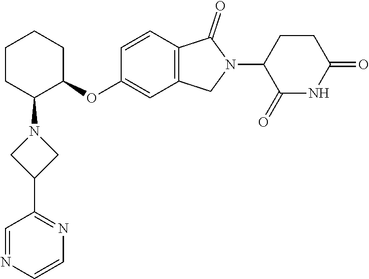 [SMILES: O=C1CCC(N2Cc3cc(O[C@@H]4CCCC[C@@H]4N4CC(c5cnccn5)C4)ccc3C2=O)C(=O)N1] | 3-(1-oxo-5-(((1R,2S)-2-(3-(pyrazin- 2-yl)azetidin-1- yl)cyclohexyl)oxy)isoindolin-2- yl)piperidine-2,6-dione |
| 43-B | 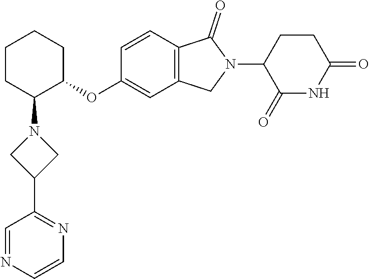 [SMILES: O=C1CCC(N2Cc3cc(O[C@H]4CCCC[C@@H]4N4CC(c5cnccn5)C4)ccc3C2=O)C(=O)N1] | 3-(1-oxo-5-(((1S,2S)-2-(3-(pyrazin- 2-yl)azetidin-1- yl)cyclohexyl)oxy)isoindolin-2- yl)piperidine-2,6-dione |
| 44-B | 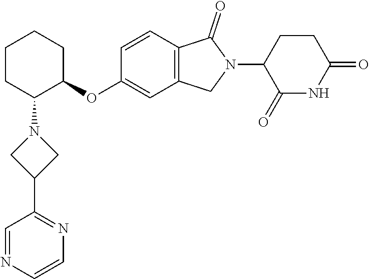 [SMILES: O=C1CCC(N2Cc3cc(O[C@@H]4CCCC[C@H]4N4CC(c5cnccn5)C4)ccc3C2=O)C(=O)N1] | 3-(1-oxo-5-(((1R,2R)-2-(3-(pyrazin- 2-yl)azetidin-1- yl)cyclohexyl)oxy)isoindolin-2- yl)piperidine-2,6-dione |
| 45-B | 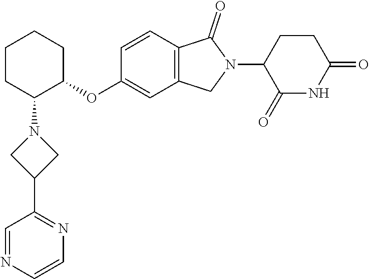 [SMILES: O=C1CCC(N2Cc3cc(O[C@H]4CCCC[C@H]4N4CC(c5cnccn5)C4)ccc3C2=O)C(=O)N1] | 3-(1-oxo-5-(((1S,2R)-2-(3-(pyrazin- 2-yl)azetidin-1- yl)cyclohexyl)oxy)isoindolin-2- yl)piperidine-2,6-dione |
| 46-B | 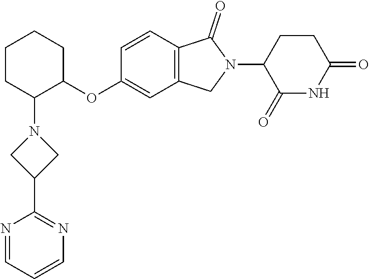 [SMILES: O=C1CCC(N2Cc3cc(OC4CCCCC4N4CC(c5ncccn5)C4)ccc3C2=O)C(=O)N1] | 3-(1-oxo-5-((2-(3-(pyrimidin-2- yl)azetidin-1- yl)cyclohexyl)oxy)isoindolin-2- yl)piperidine-2,6-dione |
| 47-B | 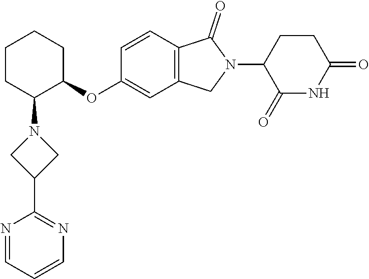 [SMILES: O=C1CCC(N2Cc3cc(O[C@@H]4CCCC[C@@H]4N4CC(c5ncccn5)C4)ccc3C2=O)C(=O)N1] | 3-(1-oxo-5-(((1R,2S)-2-(3- (pyrimidin-2-yl)azetidin-1- yl)cyclohexyl)oxy)isoindolin-2- yl)piperidine-2,6-dione |
| 48-B | 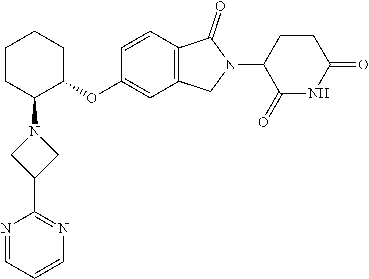 [SMILES: O=C1CCC(N2Cc3cc(O[C@H]4CCCC[C@@H]4N4CC(c5ncccn5)C4)ccc3C2=O)C(=O)N1] | 3-(1-oxo-5-(((1S,2S)-2-(3- (pyrimidin-2-yl)azetidin-1- yl)cyclohexyl)oxy)isoindolin-2- yl)piperidine-2,6-dione |
| 49-B | 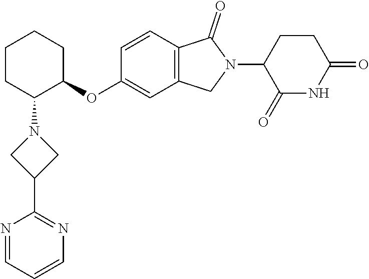 [SMILES: O=C1CCC(N2Cc3cc(O[C@@H]4CCCC[C@H]4N4CC(c5ncccn5)C4)ccc3C2=O)C(=O)N1] | 3-(1-oxo-5-(((1R,2R)-2-(3- (pyrimidin-2-yl)azetidin-1- yl)cyclohexyl)oxy)isoindolin-2- yl)piperidine-2,6-dione |
| 50-B | 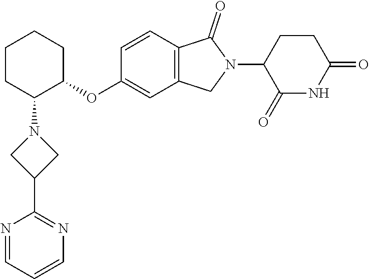 [SMILES: O=C1CCC(N2Cc3cc(O[C@H]4CCCC[C@H]4N4CC(c5ncccn5)C4)ccc3C2=O)C(=O)N1] | 3-(1-oxo-5-(((1S,2R)-2-(3- (pyrimidin-2-yl)azetidin-1- yl)cyclohexyl)oxy)isoindolin-2- yl)piperidine-2,6-dione |
| 51-B | 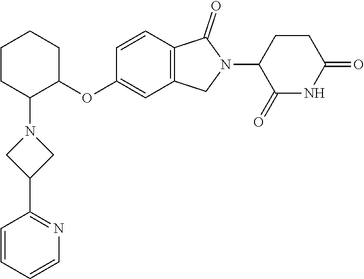 [SMILES: O=C1CCC(N2Cc3cc(OC4CCCCC4N4CC(c5ccccn5)C4)ccc3C2=O)C(=O)N1] | 3-(1-oxo-5-((2-(3-(pyridin-2- yl)azetidin-1- yl)cyclohexyl)oxy)isoindolin-2- yl)piperidine-2,6-dione |
| 52-B | 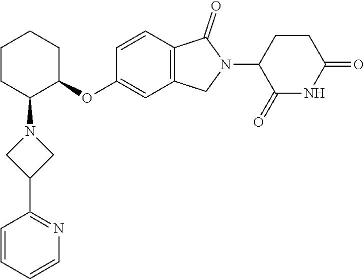 [SMILES: O=C1CCC(N2Cc3cc(O[C@@H]4CCCC[C@@H]4N4CC(c5ccccn5)C4)ccc3C2=O)C(=O)N1] | 3-(1-oxo-5-(((1R,2S)-2-(3-(pyridin- 2-yl)azetidin-1- yl)cyclohexyl)oxy)isoindolin-2- yl)piperidine-2,6-dione |
| 53-B | 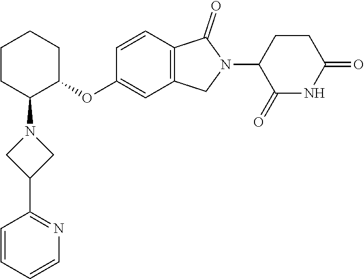 [SMILES: O=C1CCC(N2Cc3cc(O[C@H]4CCCC[C@@H]4N4CC(c5ccccn5)C4)ccc3C2=O)C(=O)N1] | 3-(1-oxo-5-(((1S,2S)-2-(3-(pyridin- 2-yl)azetidin-1- yl)cyclohexyl)oxy)isoindolin-2- yl)piperidine-2,6-dione |
| 54-B | 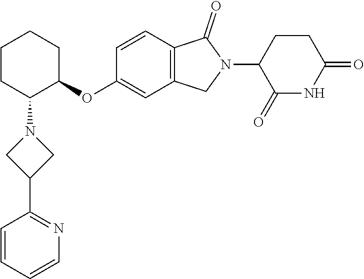 [SMILES: O=C1CCC(N2Cc3cc(O[C@@H]4CCCC[C@H]4N4CC(c5ccccn5)C4)ccc3C2=O)C(=O)N1] | 3-(1-oxo-5-(((1R,2R)-2-(3-(pyridin- 2-yl)azetidin-1- yl)cyclohexyl)oxy)isoindolin-2- yl)piperidine-2,6-dione |
| 55-B | 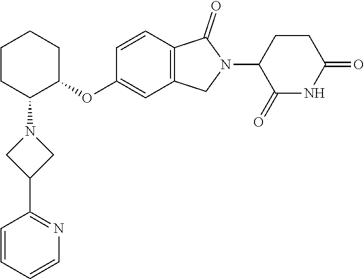 [SMILES: O=C1CCC(N2Cc3cc(O[C@H]4CCCC[C@H]4N4CC(c5ccccn5)C4)ccc3C2=O)C(=O)N1] | 3-(1-oxo-5-(((1S,2R)-2-(3-(pyridin- 2-yl)azetidin-1- yl)cyclohexyl)oxy)isoindolin-2- yl)piperidine-2,6-dione |
| 56-B | 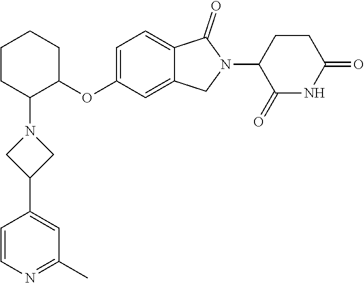 [SMILES: Cc1cc(C2CN(C3CCCCC3Oc3ccc4c(c3)CN(C3CCC(=O)NC3=O)C4=O)C2)ccn1] | 3-(5-((2-(3-(2-methylpyridin-4- yl)azetidin-1-yl)cyclohexyl)oxy)-1- oxoisoindolin-2-yl)piperidine-2,6- dione |
| 57-B | 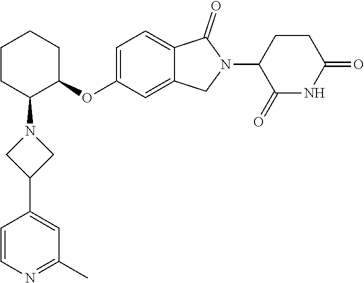 [SMILES: Cc1cc(C2CN([C@H]3CCCC[C@H]3Oc3ccc4c(c3)CN(C3CCC(=O)NC3=O)C4=O)C2)ccn1] | 3-(5-(((1R,2S)-2-(3-(2- methylpyridin-4-yl)azetidin-1- yl)cyclohexyl)oxy)-1- oxoisoindolin-2-yl)piperidine-2,6- dione |
| 58-B | 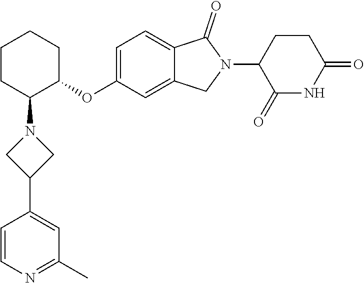 [SMILES: Cc1cc(C2CN([C@H]3CCCC[C@@H]3Oc3ccc4c(c3)CN(C3CCC(=O)NC3=O)C4=O)C2)ccn1] | 3-(5-(((1S,2S)-2-(3-(2- methylpyridin-4-yl)azetidin-1- yl)cyclohexyl)oxy)-1- oxoisoindolin-2-yl)piperidine-2,6- dione |
| 59-B | 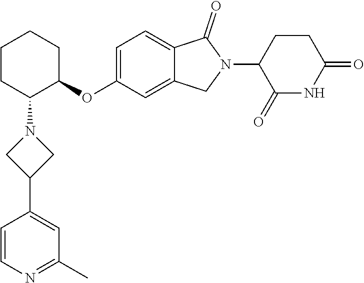 [SMILES: Cc1cc(C2CN([C@@H]3CCCC[C@H]3Oc3ccc4c(c3)CN(C3CCC(=O)NC3=O)C4=O)C2)ccn1] | 3-(5-(((1R,2R)-2-(3-(2- methylpyridin-4-yl)azetidin-1- yl)cyclohexyl)oxy)-1- oxoisoindolin-2-yl)piperidine-2,6- dione |
| 60-B | 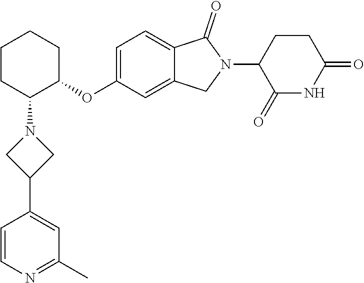 [SMILES: Cc1cc(C2CN([C@@H]3CCCC[C@@H]3Oc3ccc4c(c3)CN(C3CCC(=O)NC3=O)C4=O)C2)ccn1] | 3-(5-(((1S,2R)-2-(3-(2- methylpyridin-4-yl)azetidin-1- yl)cyclohexyl)oxy)-1- oxoisoindolin-2-yl)piperidine-2,6- dione |
| 61-B | 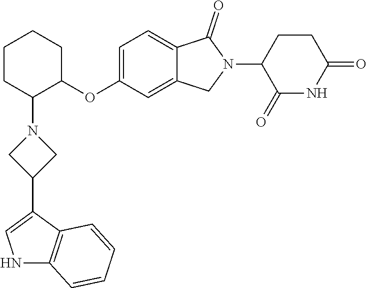 [SMILES: O=C1CCC(N2Cc3cc(OC4CCCCC4N4CC(c5c[nH]c6ccccc56)C4)ccc3C2=O)C(=O)N1] | 3-(5-((2-(3-(1H-indol-3-yl)azetidin- 1-yl)cyclohexyl)oxy)-1- oxoisoindolin-2-yl)piperidine-2,6- dione |
| 62-B | 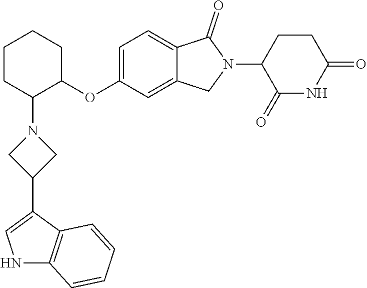 [SMILES: O=C1CCC(N2Cc3cc(OC4CCCCC4N4CC(c5c[nH]c6ccccc56)C4)ccc3C2=O)C(=O)N1] | 3-(5-(((1R,2S)-2-(3-(1H-indol-3- yl)azetidin-1-yl)cyclohexyl)oxy)-1- oxoisoindolin-2-yl)piperidine-2,6- dione |
| 63-B | 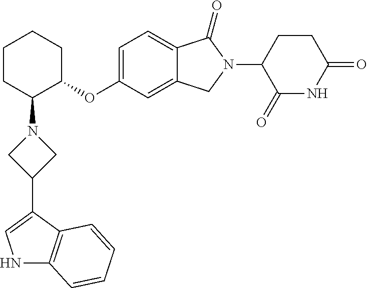 [SMILES: O=C1CCC(N2Cc3cc(O[C@H]4CCCC[C@@H]4N4CC(c5c[nH]c6ccccc56)C4)ccc3C2=O)C(=O)N1] | 3-(5-(((1S,2S)-2-(3-(1H-indol-3- yl)azetidin-1-yl)cyclohexyl)oxy)-1- oxoisoindolin-2-yl)piperidine-2,6- dione |
| 64-B | 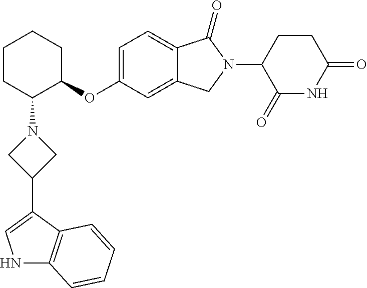 [SMILES: O=C1CCC(N2Cc3cc(O[C@@H]4CCCC[C@H]4N4CC(c5c[nH]c6ccccc56)C4)ccc3C2=O)C(=O)N1] | 3-(5-(((1R,2R)-2-(3-(1H-indol-3- yl)azetidin-1-yl)cyclohexyl)oxy)-1- oxoisoindolin-2-yl)piperidine-2,6- dione |
| 65-B | 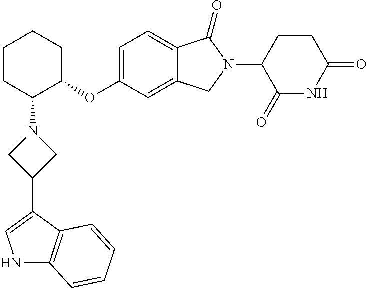 [SMILES: O=C1CCC(N2Cc3cc(O[C@H]4CCCC[C@H]4N4CC(c5c[nH]c6ccccc56)C4)ccc3C2=O)C(=O)N1] | 3-(5-(((1S,2R)-2-(3-(1H-indol-3- yl)azetidin-1-yl)cyclohexyl)oxy)-1- oxoisoindolin-2-yl)piperidine-2,6- dione |
| 66-B | 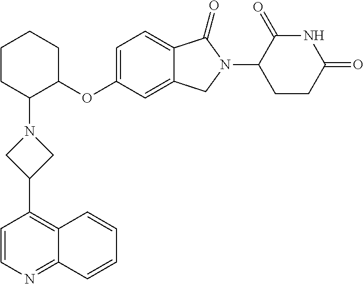 [SMILES: O=C1CCC(N2Cc3cc(OC4CCCCC4N4CC(c5ccnc6ccccc56)C4)ccc3C2=O)C(=O)N1] | 3-(1-oxo-5-((2-(3-(quinolin-4- yl)azetidin-1- yl)cyclohexyl)oxy)isoindolin-2- yl)piperidine-2,6-dione |
| 67-B | 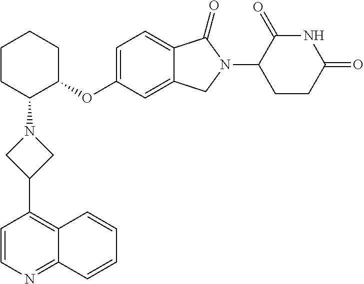 [SMILES: O=C1CCC(N2Cc3cc(O[C@H]4CCCC[C@H]4N4CC(c5ccnc6ccccc56)C4)ccc3C2=O)C(=O)N1] | 3-(1-oxo-5-(((1S,2R)-2-(3- (quinolin-4-yl)azetidin-1- yl)cyclohexyl)oxy)isoindolin-2- yl)piperidine-2,6-dione |
| 68-B | 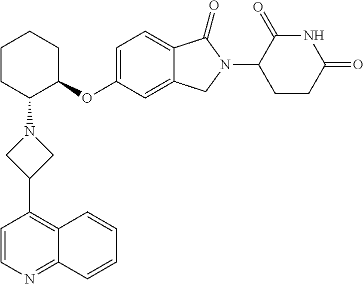 [SMILES: O=C1CCC(N2Cc3cc(O[C@@H]4CCCC[C@H]4N4CC(c5ccnc6ccccc56)C4)ccc3C2=O)C(=O)N1] | 3-(1-oxo-5-(((1R,2R)-2-(3- (quinolin-4-yl)azetidin-1- yl)cyclohexyl)oxy)isoindolin-2- yl)piperidine-2,6-dione |
| 69-B | 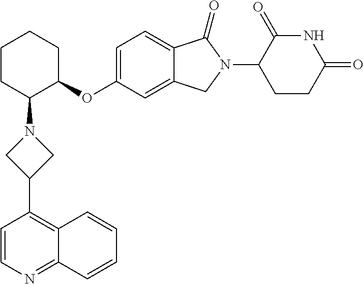 [SMILES: O=C1CCC(N2Cc3cc(O[C@@H]4CCCC[C@@H]4N4CC(c5ccnc6ccccc56)C4)ccc3C2=O)C(=O)N1] | 3-(1-oxo-5-(((1R,2S)-2-(3- (quinolin-4-yl)azetidin-1- yl)cyclohexyl)oxy)isoindolin-2- yl)piperidine-2,6-dione |
| 70-B | 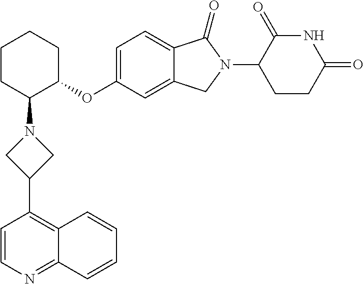 [SMILES: O=C1CCC(N2Cc3cc(O[C@H]4CCCC[C@@H]4N4CC(c5ccnc6ccccc56)C4)ccc3C2=O)C(=O)N1] | 3-(1-oxo-5-(((1S,2S)-2-(3- (quinolin-4-yl)azetidin-1- yl)cyclohexyl)oxy)isoindolin-2- yl)piperidine-2,6-dione |
| 71-B | 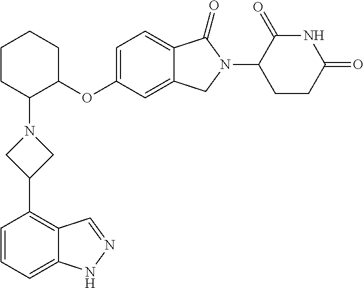 [SMILES: O=C1CCC(N2Cc3cc(OC4CCCCC4N4CC(c5cccc6[nH]ncc56)C4)ccc3C2=O)C(=O)N1] | 3-(5-((2-(3-(1H-indazol-4- yl)azetidin-1-yl)cyclohexyl)oxy)-1- oxoisoindolin-2-yl)piperidine-2,6- dione |
| 72-B | 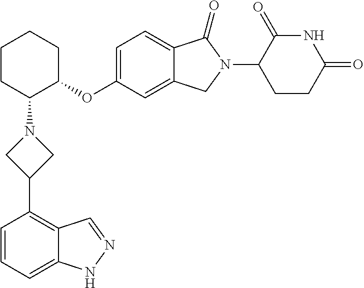 [SMILES: O=C1CCC(N2Cc3cc(O[C@H]4CCCC[C@H]4N4CC(c5cccc6[nH]ncc56)C4)ccc3C2=O)C(=O)N1] | 3-(5-(((1S,2R)-2-(3-(1H-indazol-4- yl)azetidin-1-yl)cyclohexyl)oxy)-1- oxoisoindolin-2-yl)piperidine-2,6- dione |
| 73-B | 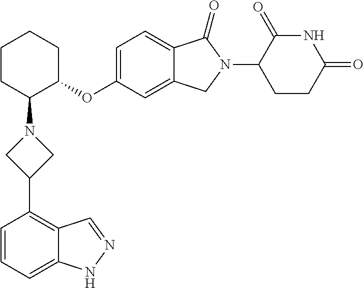 [SMILES: O=C1CCC(N2Cc3cc(O[C@H]4CCCC[C@@H]4N4CC(c5cccc6[nH]ncc56)C4)ccc3C2=O)C(=O)N1] | 3-(5-(((1S,2S)-2-(3-(1H-indazol-4- yl)azetidin-1-yl)cyclohexyl)oxy)-1- oxoisoindolin-2-yl)piperidine-2,6- dione |
| 74-B | 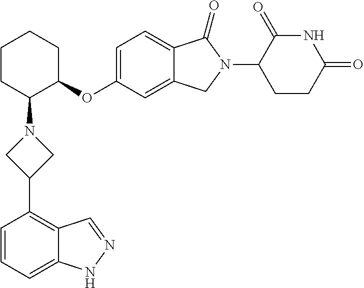 [SMILES: O=C1CCC(N2Cc3cc(O[C@@H]4CCCC[C@@H]4N4CC(c5cccc6[nH]ncc56)C4)ccc3C2=O)C(=O)N1] | 3-(5-(((1R,2S)-2-(3-(1H-indazol-4- yl)azetidin-1-yl)cyclohexyl)oxy)-1- oxoisoindolin-2-yl)piperidine-2,6- dione |
| 75-B | 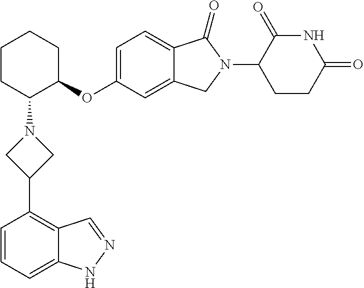 [SMILES: O=C1CCC(N2Cc3cc(O[C@@H]4CCCC[C@H]4N4CC(c5cccc6[nH]ncc56)C4)ccc3C2=O)C(=O)N1] | 3-(5-(((1R,2R)-2-(3-(1H-indazol-4- yl)azetidin-1-yl)cyclohexyl)oxy)-1- oxoisoindolin-2-yl)piperidine-2,6- dione |
| 76-B | 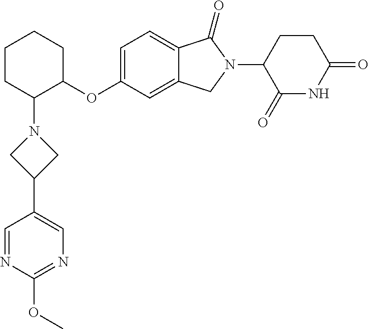 [SMILES: COc1ncc(C2CN(C3CCCCC3Oc3ccc4c(c3)CN(C3CCC(=O)NC3=O)C4=O)C2)cn1] | 3-(5-((2-(3-(2-methoxypyrimidin-5- yl)azetidin-1-yl)cyclohexyl)oxy)-1- oxoisoindolin-2-yl)piperidine-2,6- dione |
| 77-B | 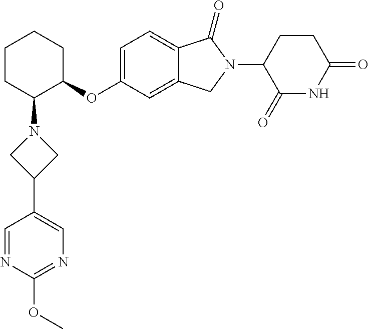 [SMILES: COc1ncc(C2CN([C@H]3CCCC[C@H]3Oc3ccc4c(c3)CN(C3CCC(=O)NC3=O)C4=O)C2)cn1] | 3-(5-(((1R,2S)-2-(3-(2- methoxypyrimidin-5-yl)azetidin-1- yl)cyclohexyl)oxy)-1- oxoisoindolin-2-yl)piperidine-2,6- dione |
| 78-B | 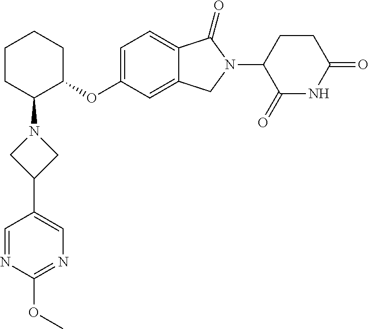 [SMILES: COc1ncc(C2CN([C@H]3CCCC[C@@H]3Oc3ccc4c(c3)CN(C3CCC(=O)NC3=O)C4=O)C2)cn1] | 3-(5-(((1S,2S)-2-(3-(2- methoxypyrimidin-5-yl)azetidin-1- yl)cyclohexyl)oxy)-1- oxoisoindolin-2-yl)piperidine-2,6- dione |
| 79-B | 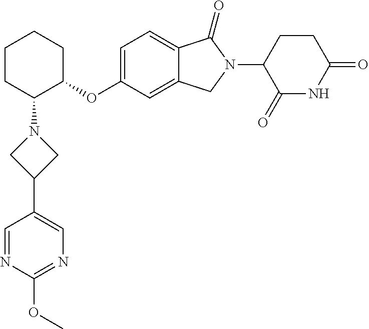 [SMILES: COc1ncc(C2CN([C@@H]3CCCC[C@@H]3Oc3ccc4c(c3)CN(C3CCC(=O)NC3=O)C4=O)C2)cn1] | 3-(5-(((1S,2R)-2-(3-(2- methoxypyrimidin-5-yl)azetidin-1- yl)cyclohexyl)oxy)-1- oxoisoindolin-2-yl)piperidine-2,6- dione |
| 80-B | 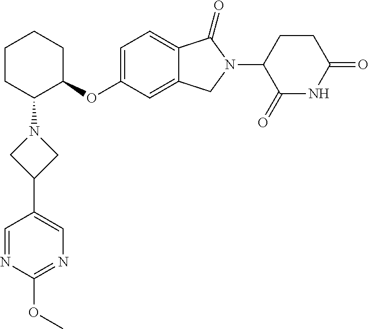 [SMILES: COc1ncc(C2CN([C@@H]3CCCC[C@H]3Oc3ccc4c(c3)CN(C3CCC(=O)NC3=O)C4=O)C2)cn1] | 3-(5-(((1R,2R)-2-(3-(2- methoxypyrimidin-5-yl)azetidin-1- yl)cyclohexyl)oxy)-1- oxoisoindolin-2-yl)piperidine-2,6- dione |
| 81-B | 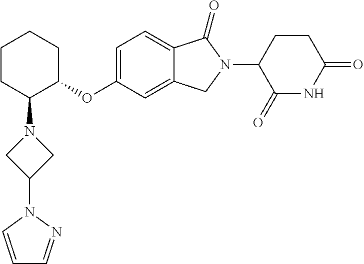 [SMILES: O=C1CCC(N2Cc3cc(O[C@H]4CCCC[C@@H]4N4CC(n5cccn5)C4)ccc3C2=O)C(=O)N1] | 3-(5-(((1S,2S)-2-(3-(1H-pyrazol-1- yl)azetidin-1-yl)cyclohexyl)oxy)-1- oxoisoindolin-2-yl)piperidine-2,6- dione |
| 82-B | 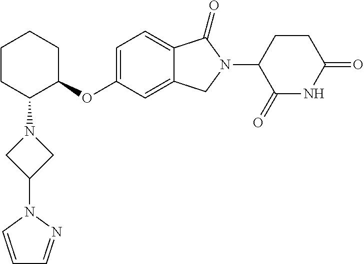 [SMILES: O=C1CCC(N2Cc3cc(O[C@@H]4CCCC[C@H]4N4CC(n5cccn5)C4)ccc3C2=O)C(=O)N1] | 3-(5-(((1R,2R)-2-(3-(1H-pyrazol-1- yl)azetidin-1-yl)cyclohexyl)oxy)-1- oxoisoindolin-2-yl)piperidine-2,6- dione |
| 83-B | 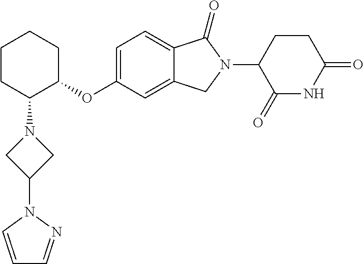 [SMILES: O=C1CCC(N2Cc3cc(O[C@H]4CCCC[C@H]4N4CC(n5cccn5)C4)ccc3C2=O)C(=O)N1] | 3-(5-(((1S,2R)-2-(3-(1H-pyrazol-1- yl)azetidin-1-yl)cyclohexyl)oxy)-1- oxoisoindolin-2-yl)piperidine-2,6- dione |
| 84-B | 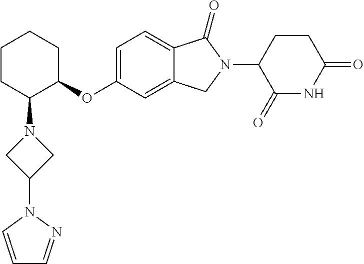 [SMILES: O=C1CCC(N2Cc3cc(O[C@@H]4CCCC[C@@H]4N4CC(n5cccn5)C4)ccc3C2=O)C(=O)N1] | 3-(5-(((1R,2S)-2-(3-(1H-pyrazol-1- yl)azetidin-1-yl)cyclohexyl)oxy)-1- oxoisoindolin-2-yl)piperidine-2,6- dione |
| 85-B | 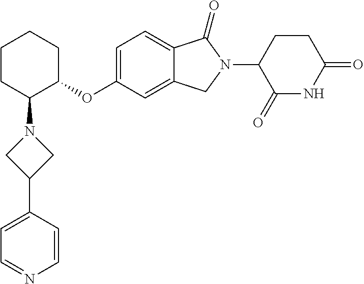 [SMILES: O=C1CCC(N2Cc3cc(O[C@H]4CCCC[C@@H]4N4CC(c5ccncc5)C4)ccc3C2=O)C(=O)N1] | 3-(1-oxo-5-(((1S,2S)-2-(3-(pyridin- 4-yl)azetidin-1- yl)cyclohexyl)oxy)isoindolin-2- yl)piperidine-2,6-dione |
| | Diastereomer 1 | |
| 86-B | 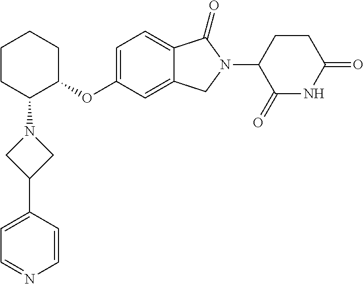 [SMILES: O=C1CCC(N2Cc3cc(O[C@H]4CCCC[C@H]4N4CC(c5ccncc5)C4)ccc3C2=O)C(=O)N1] | 3-(1-oxo-5-(((1S,2R)-2-(3-(pyridin- 4-yl)azetidin-1- yl)cyclohexyl)oxy)isoindolin-2- yl)piperidine-2,6-dione |
| | Diastereomer 2 | |
| 87-B | 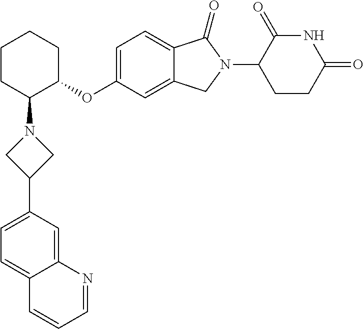 [SMILES: O=C1CCC(N2Cc3cc(O[C@H]4CCCC[C@@H]4N4CC(c5ccc6cccnc6c5)C4)ccc3C2=O)C(=O)N1] | 3-(1-oxo-5-(((1S,2S)-2-(3- (quinolin-7-yl)azetidin-1- yl)cyclohexyl)oxy)isoindolin-2- yl)piperidine-2,6-dione |
| 88-B | 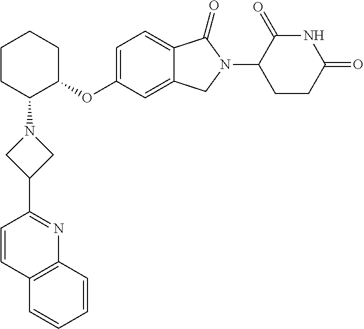 [SMILES: O=C1CCC(N2Cc3cc(O[C@H]4CCCC[C@H]4N4CC(c5ccc6ccccc6n5)C4)ccc3C2=O)C(=O)N1] | 3-(1-oxo-5-(((1S,2R)-2-(3- (quinolin-2-yl)azetidin-1- yl)cyclohexyl)oxy)isoindolin-2- yl)piperidine-2,6-dione |
| 89-B | 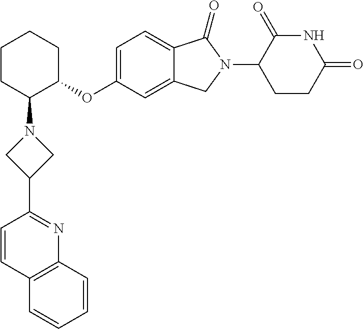 [SMILES: O=C1CCC(N2Cc3cc(O[C@H]4CCCC[C@@H]4N4CC(c5ccc6ccccc6n5)C4)ccc3C2=O)C(=O)N1] | 3-(1-oxo-5-(((1S,2S)-2-(3- (quinolin-2-yl)azetidin-1- yl)cyclohexyl)oxy)isoindolin-2- yl)piperidine-2,6-dione |
| | Diastereomer 1 | |
| 90-B | 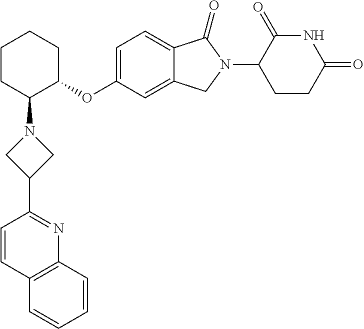 [SMILES: O=C1CCC(N2Cc3cc(O[C@H]4CCCC[C@@H]4N4CC(c5ccc6ccccc6n5)C4)ccc3C2=O)C(=O)N1] | 3-(1-oxo-5-(((1S,2S)-2-(3- (quinolin-2-yl)azetidin-1- yl)cyclohexyl)oxy)isoindolin-2- yl)piperidine-2,6-dione |
| | Diastereomer 2 | |
| 91-B | 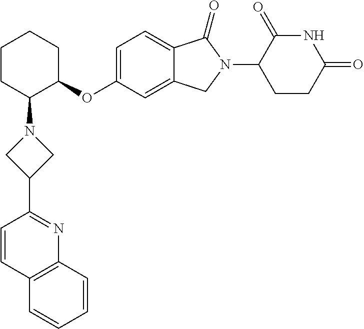 [SMILES: O=C1CCC(N2Cc3cc(O[C@@H]4CCCC[C@@H]4N4CC(c5ccc6ccccc6n5)C4)ccc3C2=O)C(=O)N1] | 3-(1-oxo-5-(((1R,2S)-2-(3- (quinolin-2-yl)azetidin-1- yl)cyclohexyl)oxy)isoindolin-2- yl)piperidine-2,6-dione |
| | Diastereomer 3 | |
| 92-B | 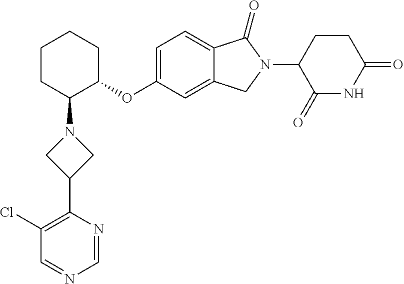 [SMILES: O=C1CCC(N2Cc3cc(O[C@H]4CCCC[C@@H]4N4CC(c5ncncc5Cl)C4)ccc3C2=O)C(=O)N1] | 3-(5-(((1S,2S)-2-(3-(5- chloropyrimidin-4-yl)azetidin-1- yl)cyclohexyl)oxy)-1- oxoisoindolin-2-yl)piperidine-2,6- dione |
| | Diastereomer 1 | |
| 93-B | 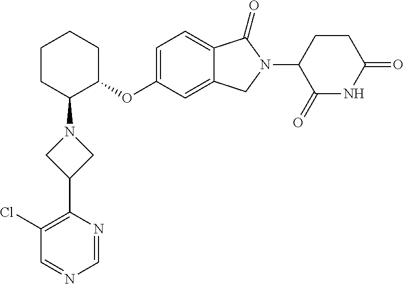 [SMILES: O=C1CCC(N2Cc3cc(O[C@H]4CCCC[C@@H]4N4CC(c5ncncc5Cl)C4)ccc3C2=O)C(=O)N1] | 3-(5-(((1S,2S)-2-(3-(5- chloropyrimidin-4-yl)azetidin-1- yl)cyclohexyl)oxy)-1- oxoisoindolin-2-yl)piperidine-2,6- dione |
| | Diastereomer 2 | |
| 94-B | 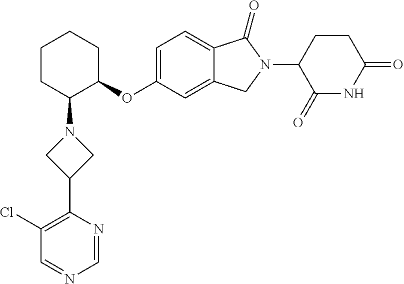 [SMILES: O=C1CCC(N2Cc3cc(O[C@@H]4CCCC[C@@H]4N4CC(c5ncncc5Cl)C4)ccc3C2=O)C(=O)N1] | 3-(5-(((1R,2S)-2-(3-(5- chloropyrimidin-4-yl)azetidin-1- yl)cyclohexyl)oxy)-1- oxoisoindolin-2-yl)piperidine-2,6- dione |
| | Diastereomer 3 | |
| 95-B | 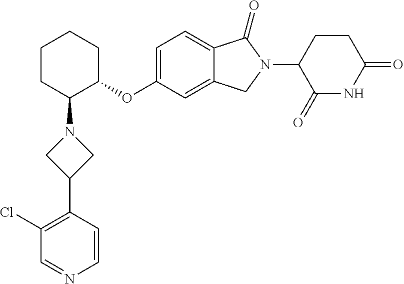 [SMILES: O=C1CCC(N2Cc3cc(O[C@H]4CCCC[C@@H]4N4CC(c5ccncc5Cl)C4)ccc3C2=O)C(=O)N1] | 3-(5-(((1S,2S)-2-(3-(3- chloropyridin-4-yl)azetidin-1- yl)cyclohexyl)oxy)-1- oxoisoindolin-2-yl)piperidine-2,6- dione |
| | Diastereomer 1 | |
| 96-B | 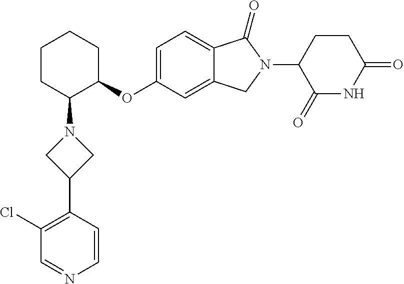 [SMILES: O=C1CCC(N2Cc3cc(O[C@@H]4CCCC[C@@H]4N4CC(c5ccncc5Cl)C4)ccc3C2=O)C(=O)N1] | 3-(5-(((1R,2S)-2-(3-(3- chloropyridin-4-yl)azetidin-1- yl)cyclohexyl)oxy)-1- oxoisoindolin-2-yl)piperidine-2,6- dione |
| | Diastereomer 2 | |
| 97-B | 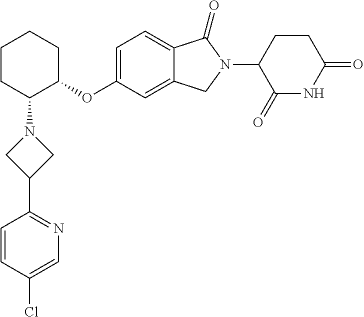 [SMILES: O=C1CCC(N2Cc3cc(O[C@H]4CCCC[C@H]4N4CC(c5ccc(Cl)cn5)C4)ccc3C2=O)C(=O)N1] | 3-(5-(((1S,2R)-2-(3-(5- chloropyridin-2-yl)azetidin-1- yl)cyclohexyl)oxy)-1- oxoisoindolin-2-yl)piperidine-2,6- dione |
| | Diastereomer 1 | |
| 98-B | 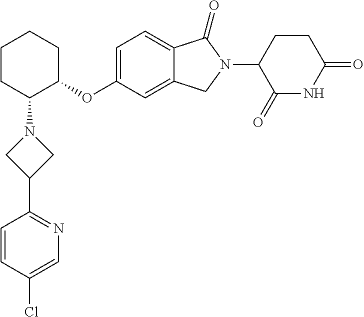 [SMILES: O=C1CCC(N2Cc3cc(O[C@H]4CCCC[C@H]4N4CC(c5ccc(Cl)cn5)C4)ccc3C2=O)C(=O)N1] | 3-(5-(((1S,2R)-2-(3-(5- chloropyridin-2-yl)azetidin-1- yl)cyclohexyl)oxy)-1- oxoisoindolin-2-yl)piperidine-2,6- dione |
| | Diastereomer 2 | |
| 99-B | 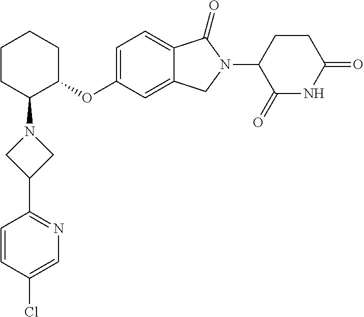 [SMILES: O=C1CCC(N2Cc3cc(O[C@H]4CCCC[C@@H]4N4CC(c5ccc(Cl)cn5)C4)ccc3C2=O)C(=O)N1] | 3-(5-(((1S,2S)-2-(3-(5- chloropyridin-2-yl)azetidin-1- yl)cyclohexyl)oxy)-1- oxoisoindolin-2-yl)piperidine-2,6- dione |
| | Diastereomer 3 | |
| 100-B | 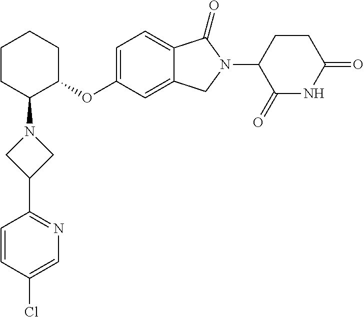 [SMILES: O=C1CCC(N2Cc3cc(O[C@H]4CCCC[C@@H]4N4CC(c5ccc(Cl)cn5)C4)ccc3C2=O)C(=O)N1] | 3-(5-(((1S,2S)-2-(3-(5- chloropyridin-2-yl)azetidin-1- yl)cyclohexyl)oxy)-1- oxoisoindolin-2-yl)piperidine-2,6- dione |
| | Diastereomer 4 | |
| 101-B | 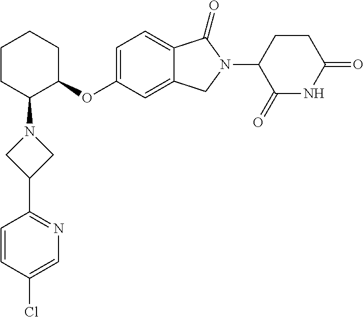 [SMILES: O=C1CCC(N2Cc3cc(O[C@@H]4CCCC[C@@H]4N4CC(c5ccc(Cl)cn5)C4)ccc3C2=O)C(=O)N1] | 3-(5-(((1R,2S)-2-(3-(5- chloropyridin-2-yl)azetidin-1- yl)cyclohexyl)oxy)-1- oxoisoindolin-2-yl)piperidine-2,6- dione |
| | Diastereomer 5 | |
| 102-B | 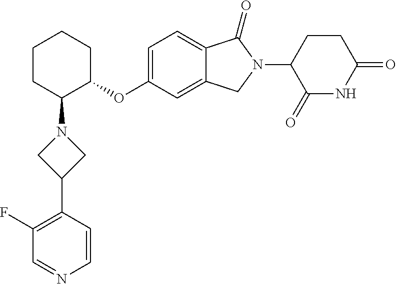 [SMILES: O=C1CCC(N2Cc3cc(O[C@H]4CCCC[C@@H]4N4CC(c5ccncc5F)C4)ccc3C2=O)C(=O)N1] | 3-(5-(((1S,2S)-2-(3-(3- fluoropyridin-4-yl)azetidin-1- yl)cyclohexyl)oxy)-1- oxoisoindolin-2-yl)piperidine-2,6- dione |
| | Diastereomer 1 | |
| 103-B | 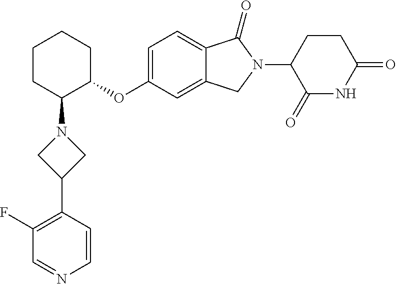 [SMILES: O=C1CCC(N2Cc3cc(O[C@H]4CCCC[C@@H]4N4CC(c5ccncc5F)C4)ccc3C2=O)C(=O)N1] | 3-(5-(((1S,2S)-2-(3-(3- fluoropyridin-4-yl)azetidin-1- yl)cyclohexyl)oxy)-1- oxoisoindolin-2-yl)piperidine-2,6- dione |
| | Diastereomer 2 | |
| 104-B | 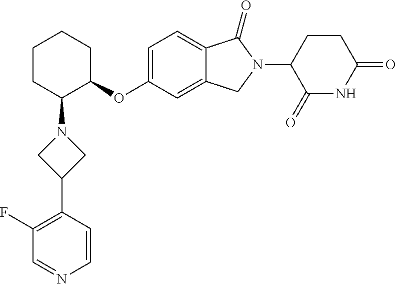 [SMILES: O=C1CCC(N2Cc3cc(O[C@@H]4CCCC[C@@H]4N4CC(c5ccncc5F)C4)ccc3C2=O)C(=O)N1] | 3-(5-(((1R,2S)-2-(3-(3- fluoropyridin-4-yl)azetidin-1- yl)cyclohexyl)oxy)-1- oxoisoindolin-2-yl)piperidine-2,6- dione |
| | Diastereomer 3 | |
| 105-B | 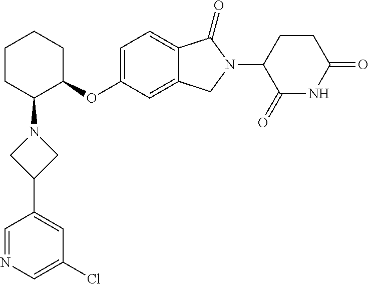 [SMILES: O=C1CCC(N2Cc3cc(O[C@@H]4CCCC[C@@H]4N4CC(c5cncc(Cl)c5)C4)ccc3C2=O)C(=O)N1] | 3-(5-(((1R,2S)-2-(3-(5- chloropyridin-3-yl)azetidin-1- yl)cyclohexyl)oxy)-1- oxoisoindolin-2-yl)piperidine-2,6- dione |
| 106-B | 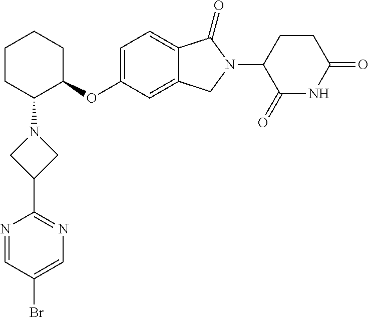 [SMILES: O=C1CCC(N2Cc3cc(O[C@@H]4CCCC[C@H]4N4CC(c5ncc(Br)cn5)C4)ccc3C2=O)C(=O)N1] | 3-(5-(((1R,2R)-2-(3-(5- bromopyrimidin-2-yl)azetidin-1- yl)cyclohexyl)oxy)-1- oxoisoindolin-2-yl)piperidine-2,6- dione |
| | Diastereomer 1 | |
| 107-B | 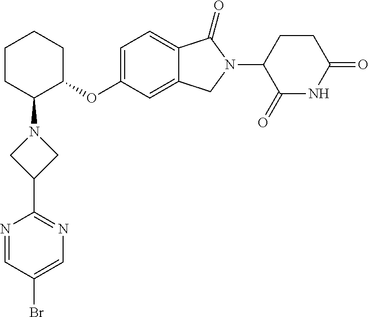 [SMILES: O=C1CCC(N2Cc3cc(O[C@H]4CCCC[C@@H]4N4CC(c5ncc(Br)cn5)C4)ccc3C2=O)C(=O)N1] | 3-(5-(((1S,2S)-2-(3-(5- bromopyrimidin-2-yl)azetidin-1- yl)cyclohexyl)oxy)-1- oxoisoindolin-2-yl)piperidine-2,6- dione |
| | Diastereomer 2 | |
| 108-B | 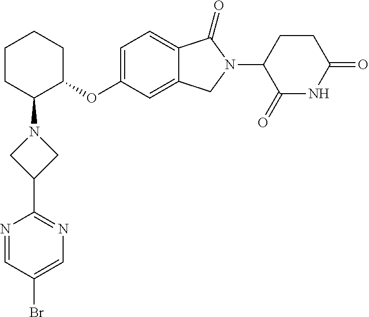 [SMILES: O=C1CCC(N2Cc3cc(O[C@H]4CCCC[C@@H]4N4CC(c5ncc(Br)cn5)C4)ccc3C2=O)C(=O)N1] | 3-(5-(((1S,2S)-2-(3-(5- bromopyrimidin-2-yl)azetidin-1- yl)cyclohexyl)oxy)-1- oxoisoindolin-2-yl)piperidine-2,6- dione |
| | Diastereomer 3 | |
| 109-B | 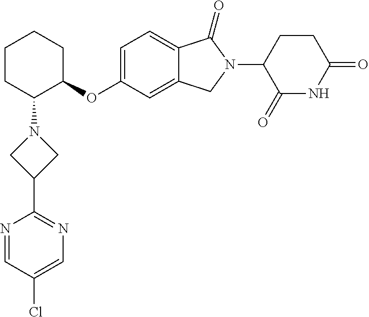 [SMILES: O=C1CCC(N2Cc3cc(O[C@@H]4CCCC[C@H]4N4CC(c5ncc(Cl)cn5)C4)ccc3C2=O)C(=O)N1] | 3-(5-(((1R,2R)-2-(3-(5- chloropyrimidin-2-yl)azetidin-1- yl)cyclohexyl)oxy)-1- oxoisoindolin-2-yl)piperidine-2,6- dione |
| | Diastereomer 1 | |
| 110-B | 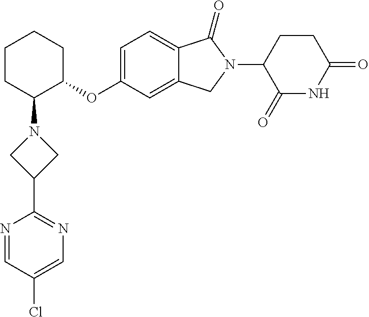 [SMILES: O=C1CCC(N2Cc3cc(O[C@H]4CCCC[C@@H]4N4CC(c5ncc(Cl)cn5)C4)ccc3C2=O)C(=O)N1] | 3-(5-(((1S,2S)-2-(3-(5- chloropyrimidin-2-yl)azetidin-1- yl)cyclohexyl)oxy)-1- oxoisoindolin-2-yl)piperidine-2,6- dione |
| | Diastereomer 2 | |
| 111-B | 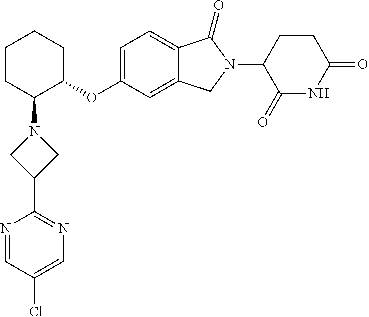 [SMILES: O=C1CCC(N2Cc3cc(O[C@H]4CCCC[C@@H]4N4CC(c5ncc(Cl)cn5)C4)ccc3C2=O)C(=O)N1] | 3-(5-(((1S,2S)-2-(3-(5- chloropyrimidin-2-yl)azetidin-1- yl)cyclohexyl)oxy)-1- oxoisoindolin-2-yl)piperidine-2,6- dione |
| | Diastereomer 3 | |
| 112-B | 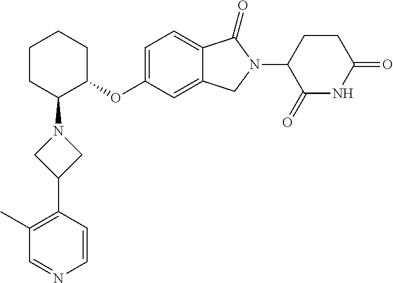 [SMILES: Cc1cnccc1C1CN([C@H]2CCCC[C@@H]2Oc2ccc3c(c2)CN(C2CCC(=O)NC2=O)C3=O)C1] | 3-(5-(((1S,2S)-2-(3-(3- methylpyridin-4-yl)azetidin-1- yl)cyclohexyl)oxy)-1- oxoisoindolin-2-yl)piperidine-2,6- dione |
| | Diastereomer 1 | |
| 113-B | 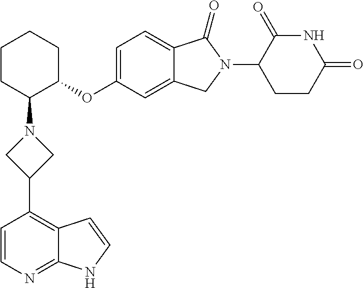 [SMILES: O=C1CCC(N2Cc3cc(O[C@H]4CCCC[C@@H]4N4CC(c5ccnc6[nH]ccc56)C4)ccc3C2=O)C(=O)N1] | 3-(5-(((1S,2S)-2-(3-(1H- pyrrolo[2,3-b]pyridin-4-yl)azetidin- 1-yl)cyclohexyl)oxy)-1- oxoisoindolin-2-yl)piperidine-2,6- dione |
| | Diastereomer 1 | |
| 114-B | 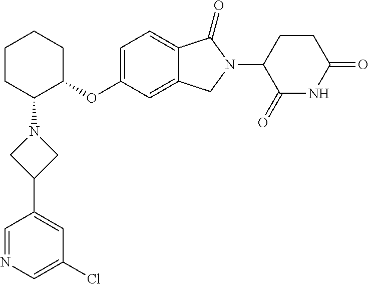 [SMILES: O=C1CCC(N2Cc3cc(O[C@H]4CCCC[C@H]4N4CC(c5cncc(Cl)c5)C4)ccc3C2=O)C(=O)N1] | 3-(5-(((1S,2R)-2-(3-(5- chloropyridin-3-yl)azetidin-1- yl)cyclohexyl)oxy)-1- oxoisoindolin-2-yl)piperidine-2,6- dione |
| | Diastereomer 2 | |
| 115-B | 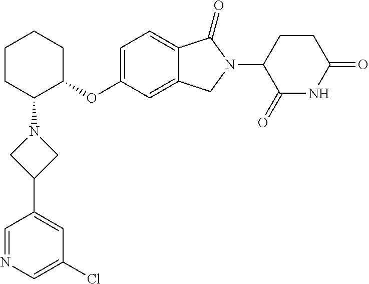 [SMILES: O=C1CCC(N2Cc3cc(O[C@H]4CCCC[C@H]4N4CC(c5cncc(Cl)c5)C4)ccc3C2=O)C(=O)N1] | 3-(5-(((1S,2R)-2-(3-(5- chloropyridin-3-yl)azetidin-1- yl)cyclohexyl)oxy)-1- oxoisoindolin-2-yl)piperidine-2,6- dione |
| | Diastereomer 3 | |
| 116-B | 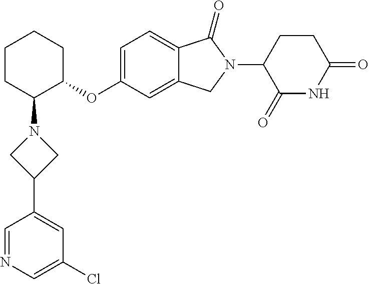 [SMILES: O=C1CCC(N2Cc3cc(O[C@H]4CCCC[C@@H]4N4CC(c5cncc(Cl)c5)C4)ccc3C2=O)C(=O)N1] | 3-(5-(((1S,2S)-2-(3-(5- chloropyridin-3-yl)azetidin-1- yl)cyclohexyl)oxy)-1- oxoisoindolin-2-yl)piperidine-2,6- dione |
| | Diastereomer 4 | |
| 117-B | 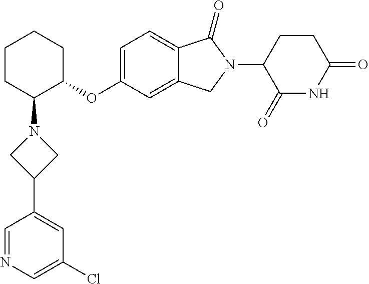 [SMILES: O=C1CCC(N2Cc3cc(O[C@H]4CCCC[C@@H]4N4CC(c5cncc(Cl)c5)C4)ccc3C2=O)C(=O)N1] | 3-(5-(((1S,2S)-2-(3-(5- chloropyridin-3-yl)azetidin-1- yl)cyclohexyl)oxy)-1- oxoisoindolin-2-yl)piperidine-2,6- dione |
| | Diastereomer 5 | |
| 118-B | 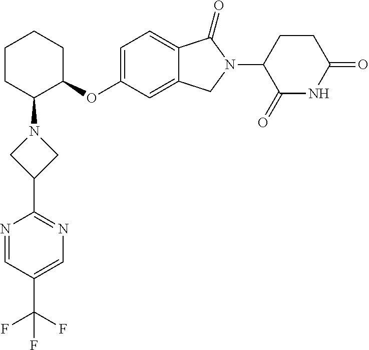 [SMILES: O=C1CCC(N2Cc3cc(O[C@@H]4CCCC[C@@H]4N4CC(c5ncc(C(F)(F)F)cn5)C4)ccc3C2=O)C(=O)N1] | 3-(1-oxo-5-(((1R,2S)-2-(3-(5- (trifluoromethyl)pyrimidin-2- yl)azetidin-1- yl)cyclohexyl)oxy)isoindolin-2- yl)piperidine-2,6-dione |
| | Diastereomer 1 | |
| 119-B | 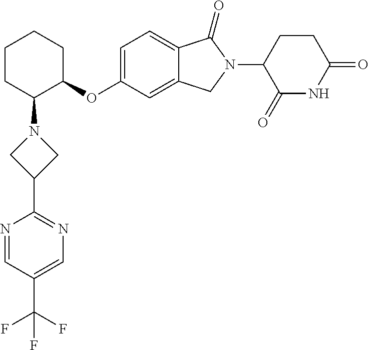 [SMILES: O=C1CCC(N2Cc3cc(O[C@@H]4CCCC[C@@H]4N4CC(c5ncc(C(F)(F)F)cn5)C4)ccc3C2=O)C(=O)N1] | 3-(1-oxo-5-(((1R,2S)-2-(3-(5- (trifluoromethyl)pyrimidin-2- yl)azetidin-1- yl)cyclohexyl)oxy)isoindolin-2- yl)piperidine-2,6-dione |
| | Diastereomer 2 | |
| 120-B | 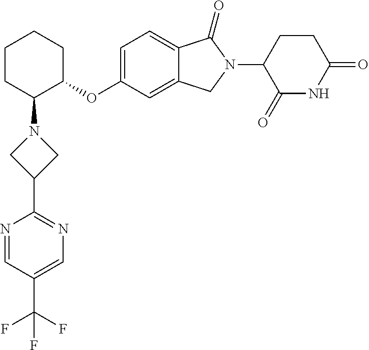 [SMILES: O=C1CCC(N2Cc3cc(O[C@H]4CCCC[C@@H]4N4CC(c5ncc(C(F)(F)F)cn5)C4)ccc3C2=O)C(=O)N1] | 3-(1-oxo-5-(((1S,2S)-2-(3-(5- (trifluoromethyl)pyrimidin-2- yl)azetidin-1- yl)cyclohexyl)oxy)isoindolin-2- yl)piperidine-2,6-dione |
| | Diastereomer 3 | |
| 121-B | 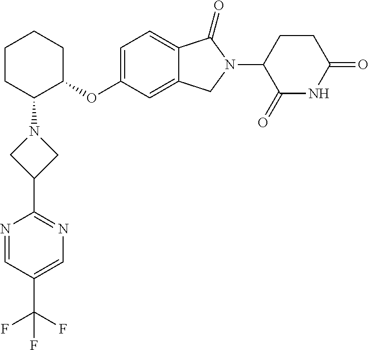 [SMILES: O=C1CCC(N2Cc3cc(O[C@H]4CCCC[C@H]4N4CC(c5ncc(C(F)(F)F)cn5)C4)ccc3C2=O)C(=O)N1] | 3-(1-oxo-5-(((1S,2R)-2-(3-(5- (trifluoromethyl)pyrimidin-2- yl)azetidin-1- yl)cyclohexyl)oxy)isoindolin-2- yl)piperidine-2,6-dione |
| | Diastereomer 4 | |
| 122-B | 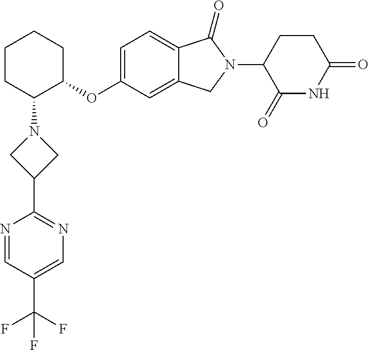 [SMILES: O=C1CCC(N2Cc3cc(O[C@H]4CCCC[C@H]4N4CC(c5ncc(C(F)(F)F)cn5)C4)ccc3C2=O)C(=O)N1] | 3-(1-oxo-5-(((1S,2R)-2-(3-(5- (trifluoromethyl)pyrimidin-2- yl)azetidin-1- yl)cyclohexyl)oxy)isoindolin-2- yl)piperidine-2,6-dione |
| | Diastereomer 5 | |
| 123-B | 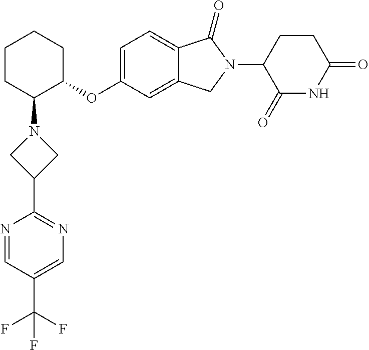 [SMILES: O=C1CCC(N2Cc3cc(O[C@H]4CCCC[C@@H]4N4CC(c5ncc(C(F)(F)F)cn5)C4)ccc3C2=O)C(=O)N1] | 3-(1-oxo-5-(((1S,2S)-2-(3-(5- (trifluoromethyl)pyrimidin-2- yl)azetidin-1- yl)cyclohexyl)oxy)isoindolin-2- yl)piperidine-2,6-dione |
| | Diastereomer 6 | |
| 124-B | 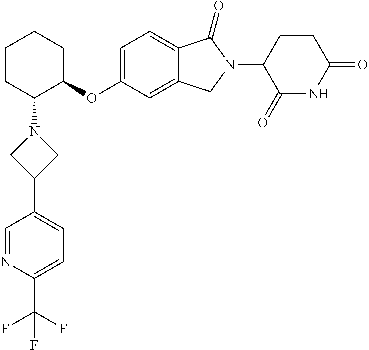 [SMILES: O=C1CCC(N2Cc3cc(O[C@@H]4CCCC[C@H]4N4CC(c5ccc(C(F)(F)F)nc5)C4)ccc3C2=O)C(=O)N1] | 3-(1-oxo-5-(((1R,2R)-2-(3-(6- (trifluoromethyl)pyridin-3- yl)azetidin-1- yl)cyclohexyl)oxy)isoindolin-2- yl)piperidine-2,6-dione |
| | Diastereomer 1 | |
| 125-B | 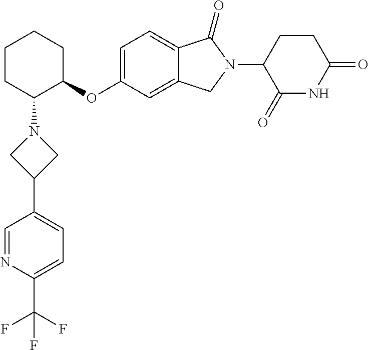 [SMILES: O=C1CCC(N2Cc3cc(O[C@@H]4CCCC[C@H]4N4CC(c5ccc(C(F)(F)F)nc5)C4)ccc3C2=O)C(=O)N1] | 3-(1-oxo-5-(((1R,2R)-2-(3-(6- (trifluoromethyl)pyridin-3- yl)azetidin-1- yl)cyclohexyl)oxy)isoindolin-2- yl)piperidine-2,6-dione |
| | Diastereomer 2 | |
| 126-B | 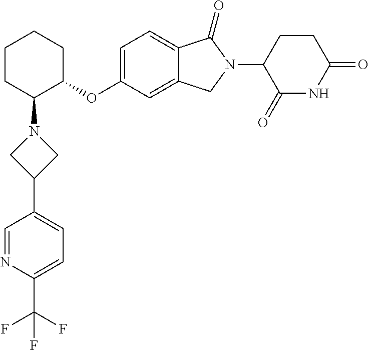 [SMILES: O=C1CCC(N2Cc3cc(O[C@H]4CCCC[C@@H]4N4CC(c5ccc(C(F)(F)F)nc5)C4)ccc3C2=O)C(=O)N1] | 3-(1-oxo-5-(((1S,2S)-2-(3-(6- (trifluoromethyl)pyridin-3- yl)azetidin-1- yl)cyclohexyl)oxy)isoindolin-2- yl)piperidine-2,6-dione |
| | Diastereomer 3 | |
| 127-B | 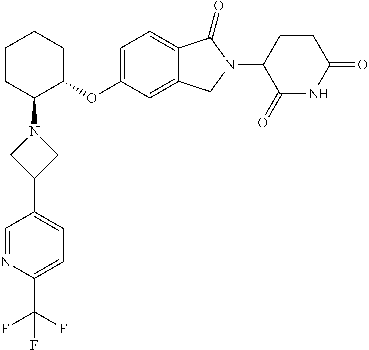 [SMILES: O=C1CCC(N2Cc3cc(O[C@H]4CCCC[C@@H]4N4CC(c5ccc(C(F)(F)F)nc5)C4)ccc3C2=O)C(=O)N1] | 3-(1-oxo-5-(((1S,2S)-2-(3-(6- (trifluoromethyl)pyridin-3- yl)azetidin-1- yl)cyclohexyl)oxy)isoindolin-2- yl)piperidine-2,6-dione |
| | Diastereomer 4 | |
| 128-B | 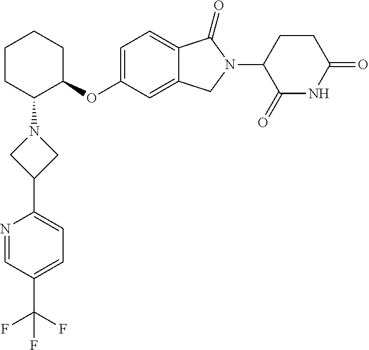 [SMILES: O=C1CCC(N2Cc3cc(O[C@@H]4CCCC[C@H]4N4CC(c5ccc(C(F)(F)F)cn5)C4)ccc3C2=O)C(=O)N1] | 3-(1-oxo-5-(((1R,2R)-2-(3-(5- (trifluoromethyl)pyridin-2- yl)azetidin-1- yl)cyclohexyl)oxy)isoindolin-2- yl)piperidine-2,6-dione |
| | Diastereomer 1 | |
| 129-B | 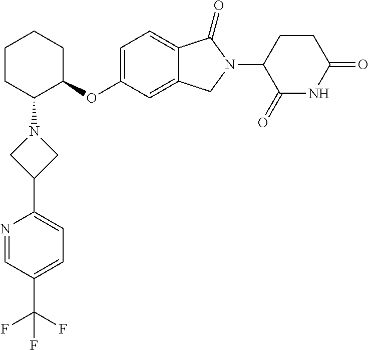 [SMILES: O=C1CCC(N2Cc3cc(O[C@@H]4CCCC[C@H]4N4CC(c5ccc(C(F)(F)F)cn5)C4)ccc3C2=O)C(=O)N1] | 3-(1-oxo-5-(((1R,2R)-2-(3-(5- (trifluoromethyl)pyridin-2- yl)azetidin-1- yl)cyclohexyl)oxy)isoindolin-2- yl)piperidine-2,6-dione |
| | Diastereomer 2 | |
| 130-B | 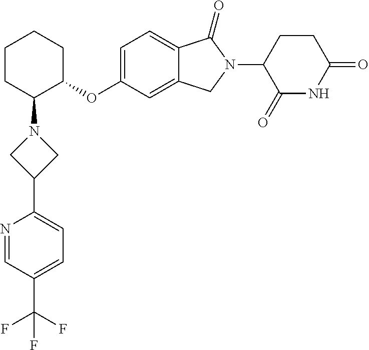 [SMILES: O=C1CCC(N2Cc3cc(O[C@H]4CCCC[C@@H]4N4CC(c5ccc(C(F)(F)F)cn5)C4)ccc3C2=O)C(=O)N1] | 3-(1-oxo-5-(((1S,2S)-2-(3-(5- (trifluoromethyl)pyridin-2- yl)azetidin-1- yl)cyclohexyl)oxy)isoindolin-2- yl)piperidine-2,6-dione |
| | Diastereomer 3 | |
| 131-B | 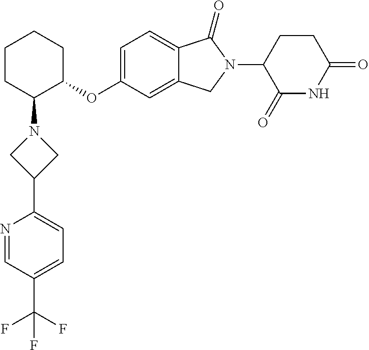 [SMILES: O=C1CCC(N2Cc3cc(O[C@H]4CCCC[C@@H]4N4CC(c5ccc(C(F)(F)F)cn5)C4)ccc3C2=O)C(=O)N1] | 3-(1-oxo-5-(((1S,2S)-2-(3-(5- (trifluoromethyl)pyridin-2- yl)azetidin-1- yl)cyclohexyl)oxy)isoindolin-2- yl)piperidine-2,6-dione |
| | Diastereomer 4 | |
| 132-B | 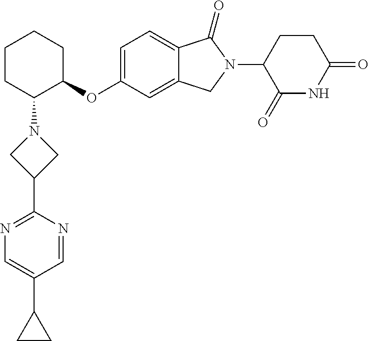 [SMILES: O=C1CCC(N2Cc3cc(O[C@@H]4CCCC[C@H]4N4CC(c5ncc(C6CC6)cn5)C4)ccc3C2=O)C(=O)N1] | 3-(5-(((1R,2R)-2-(3-(5- cyclopropylpyrimidin-2-yl)azetidin- 1-yl)cyclohexyl)oxy)-1- oxoisoindolin-2-yl)piperidine-2,6- dione |
| | Diastereomer 1 | |
| 133-B | 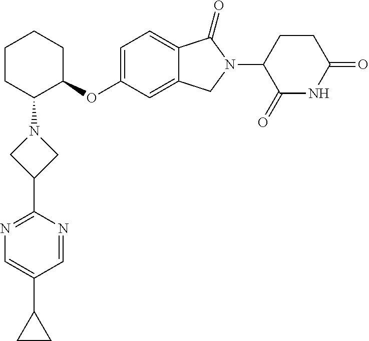 [SMILES: O=C1CCC(N2Cc3cc(O[C@@H]4CCCC[C@H]4N4CC(c5ncc(C6CC6)cn5)C4)ccc3C2=O)C(=O)N1] | 3-(5-(((1R,2R)-2-(3-(5- cyclopropylpyrimidin-2-yl)azetidin- 1-yl)cyclohexyl)oxy)-1- oxoisoindolin-2-yl)piperidine-2,6- dione |
| | Diastereomer 2 | |
| 134-B | 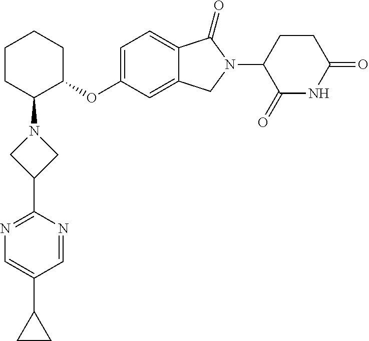 [SMILES: O=C1CCC(N2Cc3cc(O[C@H]4CCCC[C@@H]4N4CC(c5ncc(C6CC6)cn5)C4)ccc3C2=O)C(=O)N1] | 3-(5-(((1S,2S)-2-(3-(5- cyclopropylpyrimidin-2-yl)azetidin- 1-yl)cyclohexyl)oxy)-1- oxoisoindolin-2-yl)piperidine-2,6- dione |
| | Diastereomer 3 | |
| 135-B | 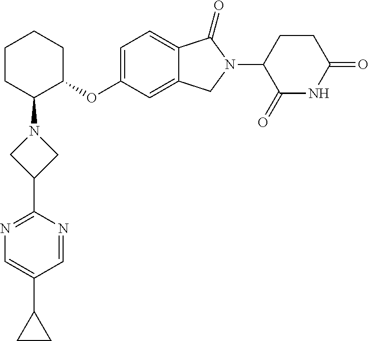 [SMILES: O=C1CCC(N2Cc3cc(O[C@H]4CCCC[C@@H]4N4CC(c5ncc(C6CC6)cn5)C4)ccc3C2=O)C(=O)N1] | 3-(5-(((1S,2S)-2-(3-(5- cyclopropylpyrimidin-2-yl)azetidin- 1-yl)cyclohexyl)oxy)-1- oxoisoindolin-2-yl)piperidine-2,6- dione |
| | Diastereomer 4 | |
| 136-B | 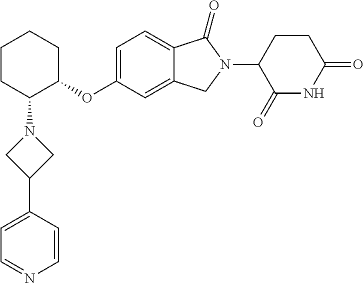 [SMILES: O=C1CCC(N2Cc3cc(O[C@H]4CCCC[C@H]4N4CC(c5ccncc5)C4)ccc3C2=O)C(=O)N1] | 3-(1-oxo-5-(((1S,2R)-2-(3-(pyridin- 4-yl)azetidin-1- yl)cyclohexyl)oxy)isoindolin-2- yl)piperidine-2,6-dione |
| | Diastereomer 3 | |
| 137-B | 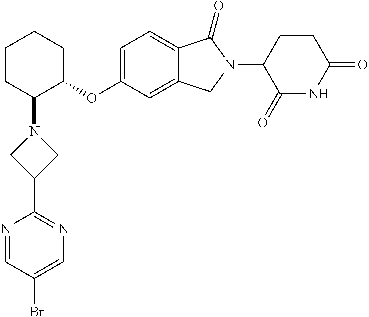 [SMILES: O=C1CCC(N2Cc3cc(O[C@H]4CCCC[C@@H]4N4CC(c5ncc(Br)cn5)C4)ccc3C2=O)C(=O)N1] | Rac-3-(5-(((trans)-2-(3-(5- bromopyrimidin-2-yl)azetidin-1- yl)cyclohexyl)oxy)-1- oxoisoindolin-2-yl)piperidine-2,6- dione |
| 138-B | 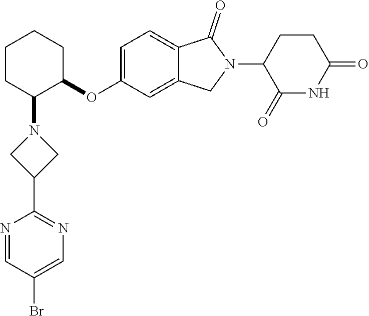 [SMILES: O=C1CCC(N2Cc3cc(O[C@@H]4CCCC[C@@H]4N4CC(c5ncc(Br)cn5)C4)ccc3C2=O)C(=O)N1] | Rac-3-(5-(((cis)-2-(3-(5- bromopyrimidin-2-yl)azetidin-1- yl)cyclohexyl)oxy)-1- oxoisoindolin-2-yl)piperidine-2,6- dione |
| 139-B | 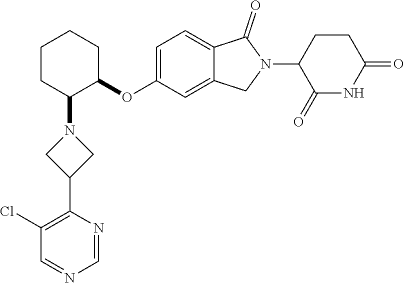 [SMILES: O=C1CCC(N2Cc3cc(O[C@@H]4CCCC[C@@H]4N4CC(c5ncncc5Cl)C4)ccc3C2=O)C(=O)N1] | Rac-3-(5-(((cis)-2-(3-(5- chloropyrimidin-4-yl)azetidin-1- yl)cyclohexyl)oxy)-1- oxoisoindolin-2-yl)piperidine-2,6- dione |
| 140-B | 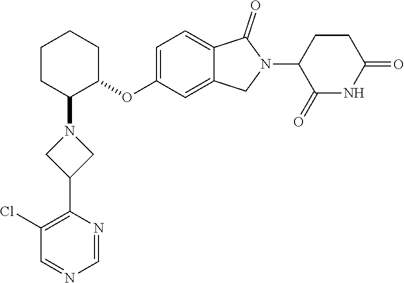 [SMILES: O=C1CCC(N2Cc3cc(O[C@H]4CCCC[C@@H]4N4CC(c5ncncc5Cl)C4)ccc3C2=O)C(=O)N1] | Rac-3-(5-(((trans)-2-(3-(5- chloropyrimidin-4-yl)azetidin-1- yl)cyclohexyl)oxy)-1- oxoisoindolin-2-yl)piperidine-2,6- dione |
| 141-B | 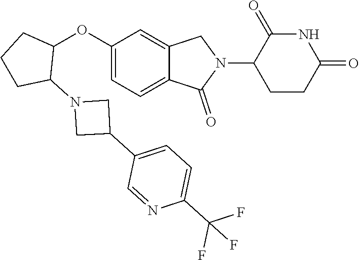 [SMILES: O=C1CCC(N2Cc3cc(OC4CCCC4N4CC(c5ccc(C(F)(F)F)nc5)C4)ccc3C2=O)C(=O)N1] | 3-(1-oxo-5-((2-(3-(6- (trifluoromethyl)pyridin-3- yl)azetidin-1- yl)cyclopentyl)oxy)isoindolin-2- yl)piperidine-2,6-dione |
| 142-B | 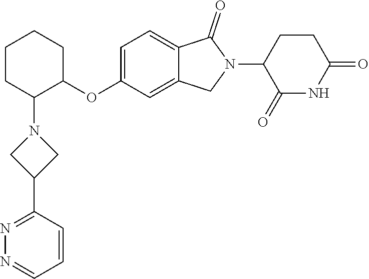 [SMILES: O=C1CCC(N2Cc3cc(OC4CCCCC4N4CC(c5cccnn5)C4)ccc3C2=O)C(=O)N1] | 3-(1-oxo-5-((2-(3-(pyridazin-3- yl)azetidin-1- yl)cyclohexyl)oxy)isoindolin-2- yl)piperidine-2,6-dione |
| 143-B | 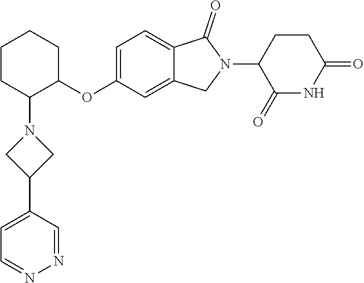 [SMILES: O=C1CCC(N2Cc3cc(OC4CCCCC4N4CC(c5ccnnc5)C4)ccc3C2=O)C(=O)N1] | 3-(1-oxo-5-((2-(3-(pyridazin-4- yl)azetidin-1- yl)cyclohexyl)oxy)isoindolin-2- yl)piperidine-2,6-dione |
| 144-B | 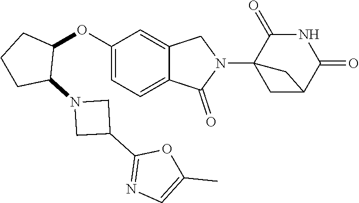 | Rac-1-(5-(((cis)-2-(3-(5- methyloxazol-2-yl)azetidin-1- yl)cyclopentyl)oxy)-1- oxoisoindolin-2-yl)-3- azabicyclo[3.1.1]heptane-2,4-dione |
| 145-B | 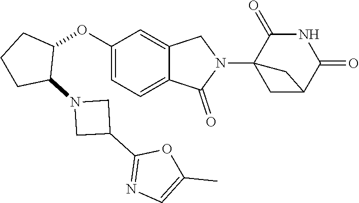 | Rac-1-(5-(((trans)-2-(3-(5- methyloxazol-2-yl)azetidin-1- yl)cyclopentyl)oxy)-1- oxoisoindolin-2-y1)-3- azabicyclo[3.1.1]heptane-2,4-dione |
| 146-B | 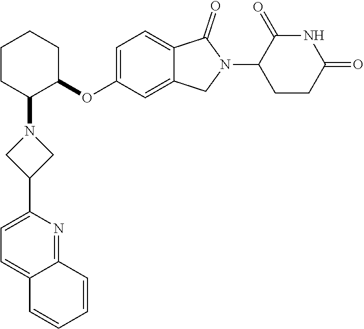 [SMILES: O=C1CCC(N2Cc3cc(O[C@@H]4CCCC[C@@H]4N4CC(c5ccc6ccccc6n5)C4)ccc3C2=O)C(=O)N1] | Rac-3-(1-oxo-5-(((cis)-2-(3- (quinolin-2-yl)azetidin-1- yl)cyclohexyl)oxy)isoindolin-2- yl)piperidine-2,6-dione |
| 147-B | 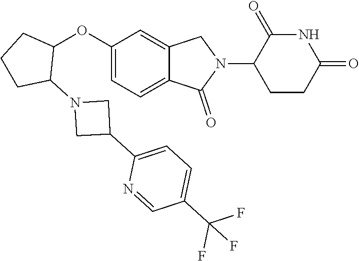 [SMILES: O=C1CCC(N2Cc3cc(OC4CCCC4N4CC(c5ccc(C(F)(F)F)cn5)C4)ccc3C2=O)C(=O)N1] | 3-(1-oxo-5-((2-(3-(5- (trifluoromethyl)pyridin-2- yl)azetidin-1- yl)cyclopentyl)oxy)isoindolin-2- yl)piperidine-2,6-dione |
| 148-B | 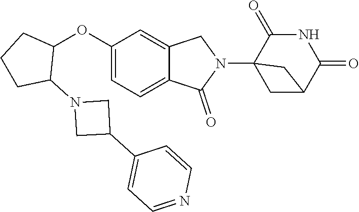 [SMILES: O=C1NC(=O)C2(N3Cc4cc(OC5CCCC5N5CC(c6ccncc6)C5)ccc4C3=O)CC1C2] | 1-(1-oxo-5-((2-(3-(pyridin-4- yl)azetidin-1- yl)cyclopentyl)oxy)isoindolin-2-yl)- 3-azabicyclo[3.1.1]heptane-2,4- dione |
| 149-B | 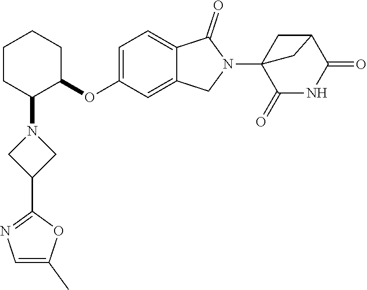 [SMILES: Cc1cnc(C2CN([C@H]3CCCC[C@H]3Oc3ccc4c(c3)CN(C35CC(C3)C(=O)NC5=O)C4=O)C2)o1] | Rac-1-(5-(((cis)-2-(3-(5- methyloxazol-2-yl)azetidin-1- yl)cyclohexyl)oxy)-1- oxoisoindolin-2-yl)-3- azabicyclo[3.1.1]heptane-2,4-dione |
| 150-B | 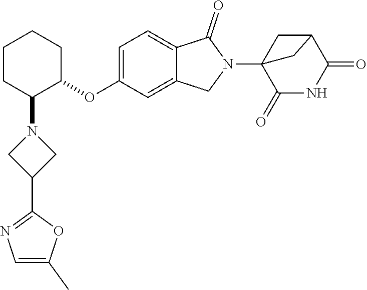 [SMILES: Cc1cnc(C2CN([C@H]3CCCC[C@@H]3Oc3ccc4c(c3)CN(C35CC(C3)C(=O)NC5=O)C4=O)C2)o1] | Rac-1-(5-(((trans)-2-(3-(5- methyloxazol-2-yl)azetidin-1- yl)cyclohexyl)oxy)-1- oxoisoindolin-2-yl)-3- azabicyclo[3.1.1]heptane-2,4-dione |
| 151-B | 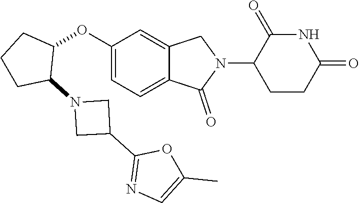 | Rac-3-(5-(((trans)-2-(3-(5- methyloxazol-2-yl)azetidin-1- yl)cyclopentyl)oxy)-1- oxoisoindolin-2-yl)piperidine-2,6- dione |
| 152-B | 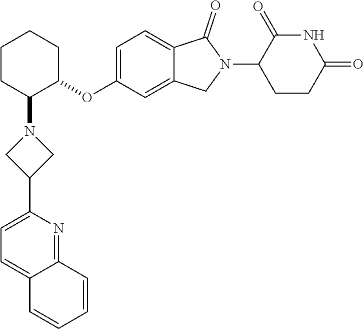 [SMILES: O=C1CCC(N2Cc3cc(O[C@H]4CCCC[C@@H]4N4CC(c5ccc6ccccc6n5)C4)ccc3C2=O)C(=O)N1] | Rac-3-(1-oxo-5-(((trans)-2-(3- (quinolin-2-yl)azetidin-1- yl)cyclohexyl)oxy)isoindolin-2- yl)piperidine-2,6-dione |
| 153-B | 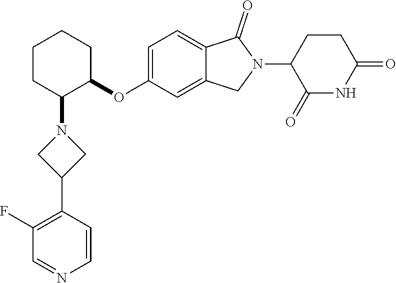 [SMILES: O=C1CCC(N2Cc3cc(O[C@@H]4CCCC[C@@H]4N4CC(c5ccncc5F)C4)ccc3C2=O)C(=O)N1] | Rac-3-(5-(((cis)-2-(3-(3- fluoropyridin-4-yl)azetidin-1- yl)cyclohexyl)oxy)-1- oxoisoindolin-2-yl)piperidine-2,6- dione |
| 154-B | 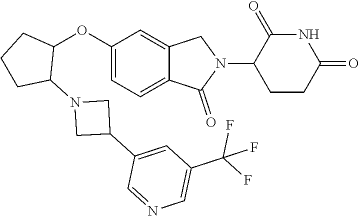 [SMILES: O=C1CCC(N2Cc3cc(OC4CCCC4N4CC(c5cncc(C(F)(F)F)c5)C4)ccc3C2=O)C(=O)N1] | 3-(1-oxo-5-((2-(3-(5- (trifluoromethyl)pyridin-3- yl)azetidin-1- yl)cyclopentyl)oxy)isoindolin-2- yl)piperidine-2,6-dione |
| 155-B | 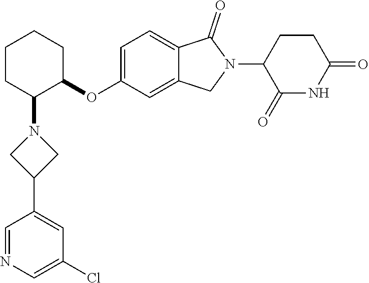 [SMILES: O=C1CCC(N2Cc3cc(O[C@@H]4CCCC[C@@H]4N4CC(c5cncc(Cl)c5)C4)ccc3C2=O)C(=O)N1] | Rac-3-(5-(((cis)-2-(3-(5- chloropyridin-3-yl)azetidin-1- yl)cyclohexyl)oxy)-1- oxoisoindolin-2-yl)piperidine-2,6- dione |
| 156-B | 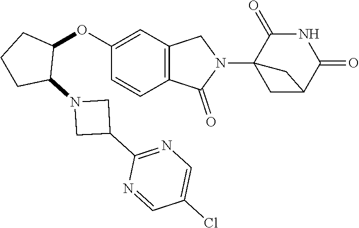 | Rac-1-(5-(((cis)-2-(3-(5- chloropyrimidin-2-yl)azetidin-1- yl)cyclopentyl)oxy)-1- oxoisoindolin-2-yl)-3- azabicyclo[3.1.1]heptane-2,4-dione |
| 157-B | 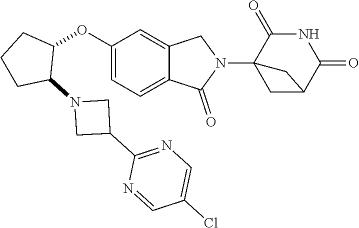 | Rac-1-(5-(((trans)-2-(3-(5- chloropyrimidin-2-yl)azetidin-1- yl)cyclopentyl)oxy)-1- oxoisoindolin-2-yl)-3- azabicyclo[3.1.1]heptane-2,4-dione |
| 158-B | 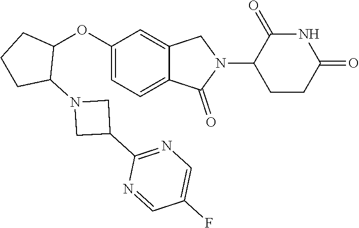 [SMILES: O=C1CCC(N2Cc3cc(OC4CCCC4N4CC(c5ncc(F)cn5)C4)ccc3C2=O)C(=O)N1] | 3-(5-((2-(3-(5-fluoropyrimidin-2- yl)azetidin-1-yl)cyclopentyl)oxy)-1- oxoisoindolin-2-yl)piperidine-2,6- dione |
| 159-B | 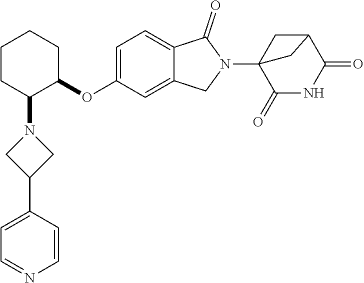 [SMILES: O=C1NC(=O)C2(N3Cc4cc(O[C@@H]5CCCC[C@@H]5N5CC(c6ccncc6)C5)ccc4C3=O)CC1C2] | Rac-1-(1-oxo-5-(((cis)-2-(3- (pyridin-4-yl)azetidin-1- yl)cyclohexyl)oxy)isoindolin-2-yl)- 3-azabicyclo[3.1.1]heptane-2,4- dione |
| 160-B | 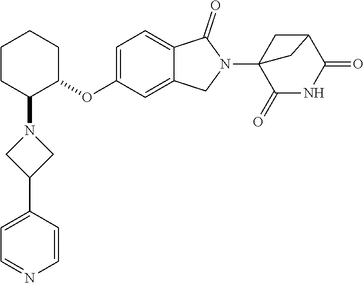 [SMILES: O=C1NC(=O)C2(N3Cc4cc(O[C@H]5CCCC[C@@H]5N5CC(c6ccncc6)C5)ccc4C3=O)CC1C2] | Rac-1-(1-oxo-5-(((trans)-2-(3- (pyridin-4-yl)azetidin-1- yl)cyclohexyl)oxy)isoindolin-2-yl)- 3-azabicyclo[3.1.1]heptane-2,4- dione |
| 161-B | 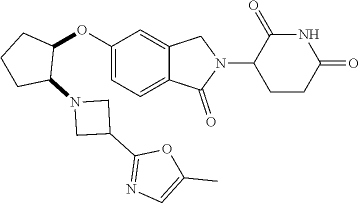 | Rac-3-(5-(((cis)-2-(3-(5- methyloxazol-2-yl)azetidin-1- yl)cyclopentyl)oxy)-1- oxoisoindolin-2-yl)piperidine-2,6- dione |
| 162-B | 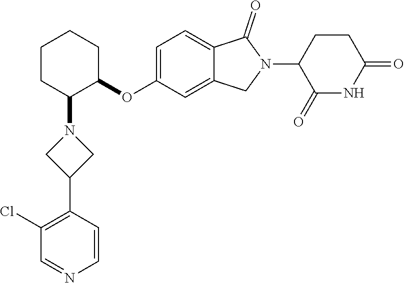 [SMILES: O=C1CCC(N2Cc3cc(O[C@@H]4CCCC[C@@H]4N4CC(c5ccncc5Cl)C4)ccc3C2=O)C(=O)N1] | Rac-3-(5-(((cis)-2-(3-(3- chloropyridin-4-yl)azetidin-1- yl)cyclohexyl)oxy)-1- oxoisoindolin-2-yl)piperidine-2,6- dione |
| 163-B | 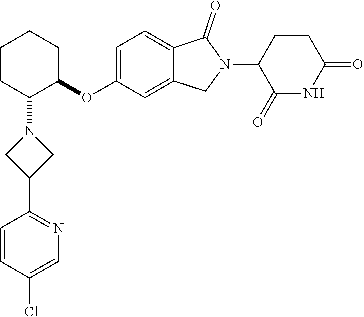 [SMILES: O=C1CCC(N2Cc3cc(O[C@@H]4CCCC[C@H]4N4CC(c5ccc(Cl)cn5)C4)ccc3C2=O)C(=O)N1] | Rac-3-(5-(((trans)-2-(3-(5- chloropyridin-2-yl)azetidin-1- yl)cyclohexyl)oxy)-1- oxoisoindolin-2-yl)piperidine-2,6- dione |
| 164-B | 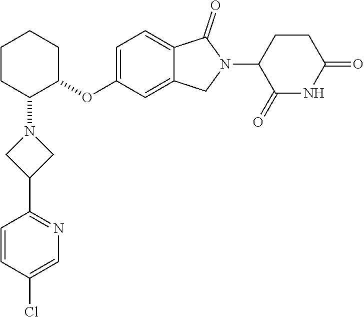 [SMILES: O=C1CCC(N2Cc3cc(O[C@H]4CCCC[C@H]4N4CC(c5ccc(Cl)cn5)C4)ccc3C2=O)C(=O)N1] | Rac-3-(5-(((cis)-2-(3-(5- chloropyridin-2-yl)azetidin-1- yl)cyclohexyl)oxy)-1- oxoisoindolin-2-yl)piperidine-2,6- dione |
| 165-B | 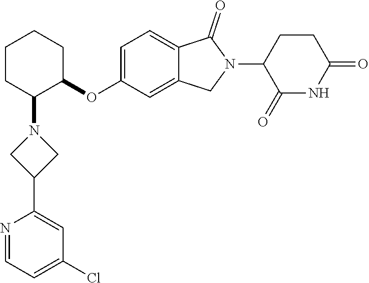 [SMILES: O=C1CCC(N2Cc3cc(O[C@@H]4CCCC[C@@H]4N4CC(c5cc(Cl)ccn5)C4)ccc3C2=O)C(=O)N1] | Rac-3-(5-(((cis)-2-(3-(4- chloropyridin-2-yl)azetidin-1- yl)cyclohexyl)oxy)-1- oxoisoindolin-2-yl)piperidine-2,6- dione |
| 166-B | 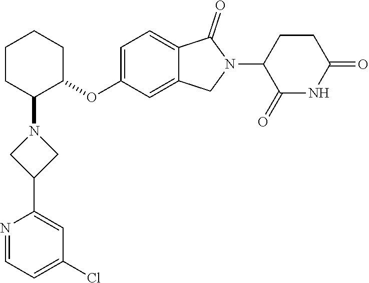 [SMILES: O=C1CCC(N2Cc3cc(O[C@H]4CCCC[C@@H]4N4CC(c5cc(Cl)ccn5)C4)ccc3C2=O)C(=O)N1] | Rac-3-(5-(((trans)-2-(3-(4- chloropyridin-2-yl)azetidin-1- yl)cyclohexyl)oxy)-1- oxoisoindolin-2-yl)piperidine-2,6- dione |
| 167-B | 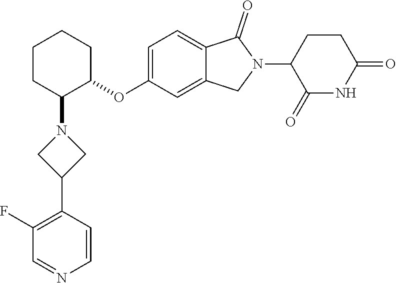 [SMILES: O=C1CCC(N2Cc3cc(O[C@H]4CCCC[C@@H]4N4CC(c5ccncc5F)C4)ccc3C2=O)C(=O)N1] | Rac-3-(5-(((trans)-2-(3-(3- fluoropyridin-4-yl)azetidin-1- yl)cyclohexyl)oxy)-1- oxoisoindolin-2-yl)piperidine-2,6- dione |
| 168-B | 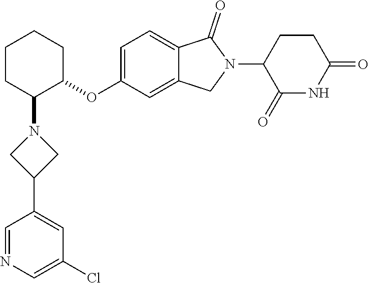 [SMILES: O=C1CCC(N2Cc3cc(O[C@H]4CCCC[C@@H]4N4CC(c5cncc(Cl)c5)C4)ccc3C2=O)C(=O)N1] | Rac-3-(5-(((trans)-2-(3-(5- chloropyridin-3-yl)azetidin-1- yl)cyclohexyl)oxy)-1- oxoisoindolin-2-yl)piperidine-2,6- dione |
| 169-B | 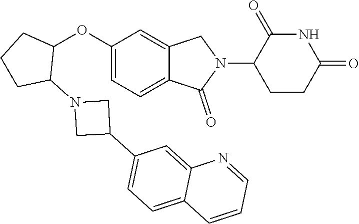 [SMILES: O=C1CCC(N2Cc3cc(OC4CCCC4N4CC(c5ccc6cccnc6c5)C4)ccc3C2=O)C(=O)N1] | 3-(1-oxo-5-((2-(3-(quinolin-7- yl)azetidin-1- yl)cyclopentyl)oxy)isoindolin-2- yl)piperidine-2,6-dione |
| 170-B | 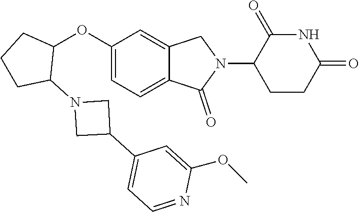 [SMILES: COc1cc(C2CN(C3CCCC3Oc3ccc4c(c3)CN(C3CCC(=O)NC3=O)C4=O)C2)ccn1] | 3-(5-((2-(3-(2-methoxypyridin-4- yl)azetidin-1-yl)cyclopentyl)oxy)-1- oxoisoindolin-2-yl)piperidine-2,6- dione |
| 171-B | 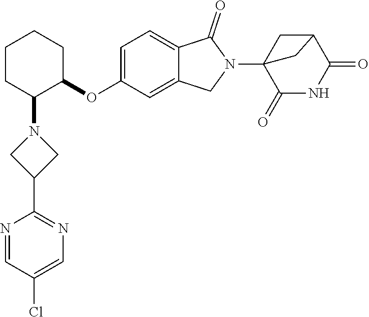 [SMILES: O=C1NC(=O)C2(N3Cc4cc(O[C@@H]5CCCC[C@@H]5N5CC(c6ncc(Cl)cn6)C5)ccc4C3=O)CC1C2] | Rac-1-(5-(((cis)-2-(3-(5- chloropyrimidin-2-yl)azetidin-1- yl)cyclohexyl)oxy)-1- oxoisoindolin-2-yl)-3- azabicyclo[3.1.1]heptane-2,4-dione |
| 172-B | 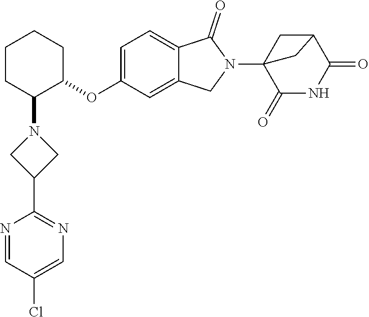 [SMILES: O=C1NC(=O)C2(N3Cc4cc(O[C@H]5CCCC[C@@H]5N5CC(c6ncc(Cl)cn6)C5)ccc4C3=O)CC1C2] | Rac-1-(5-(((trans)-2-(3-(5- chloropyrimidin-2-yl)azetidin-1- yl)cyclohexyl)oxy)-1- oxoisoindolin-2-yl)-3- azabicyclo[3.1.1]heptane-2,4-dione |
| 173-B | 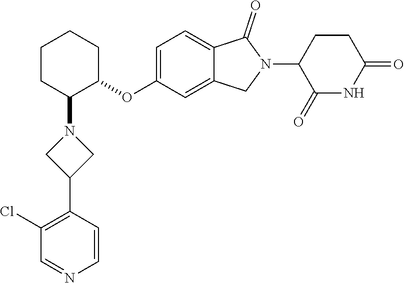 [SMILES: O=C1CCC(N2Cc3cc(O[C@H]4CCCC[C@@H]4N4CC(c5ccncc5Cl)C4)ccc3C2=O)C(=O)N1] | Rac-3-(5-(((trans)-2-(3-(3- chloropyridin-4-yl)azetidin-1- yl)cyclohexyl)oxy)-1- oxoisoindolin-2-yl)piperidine-2,6- dione |
| 174-B | 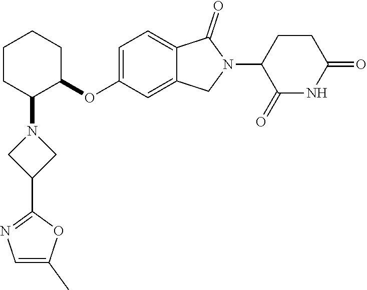 [SMILES: Cc1cnc(C2CN([C@H]3CCCC[C@H]3Oc3ccc4c(c3)CN(C3CCC(=O)NC3=O)C4=O)C2)o1] | Rac-3-(5-(((cis)-2-(3-(5- methyloxazol-2-yl)azetidin-1- yl)cyclohexyl)oxy)-1- oxoisoindolin-2-yl)piperidine-2,6- dione |
| 175-B | 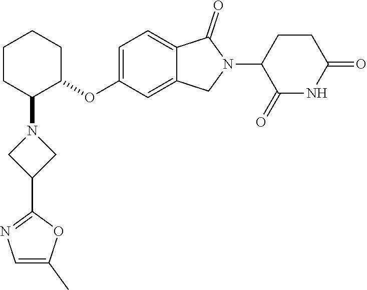 [SMILES: Cc1cnc(C2CN([C@H]3CCCC[C@@H]3Oc3ccc4c(c3)CN(C3CCC(=O)NC3=O)C4=O)C2)o1] | Rac-3-(5-(((trans)-2-(3-(5- methyloxazol-2-yl)azetidin-1- yl)cyclohexyl)oxy)-1- oxoisoindolin-2-yl)piperidine-2,6- dione |
| 176-B | 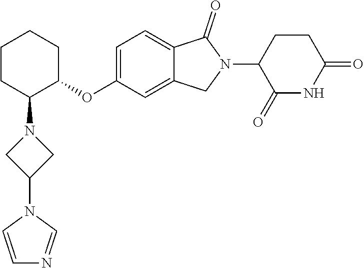 [SMILES: O=C1CCC(N2Cc3cc(O[C@H]4CCCC[C@@H]4N4CC(n5ccnc5)C4)ccc3C2=O)C(=O)N1] | 3-(5-(((1S,2S)-2-(3-(1H-imidazol- 1-yl)azetidin-1-yl)cyclohexyl)oxy)- 1-oxoisoindolin-2-yl)piperidine- 2,6-dione |
| 177-B | 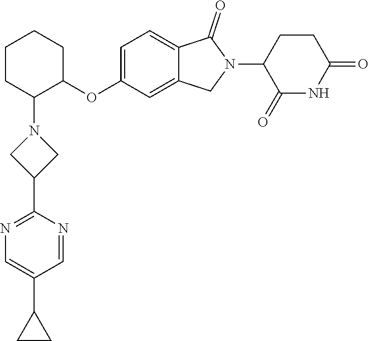 [SMILES: O=C1CCC(N2Cc3cc(OC4CCCCC4N4CC(c5ncc(C6CC6)cn5)C4)ccc3C2=O)C(=O)N1] | 3-(5-((2-(3-(5- cyclopropylpyrimidin-2-yl)azetidin- 1-yl)cyclohexyl)oxy)-1- oxoisoindolin-2-yl)piperidine-2,6- dione |
| 178-B | 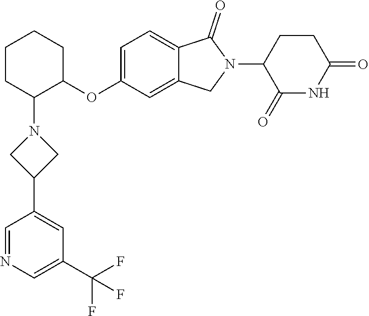 [SMILES: O=C1CCC(N2Cc3cc(OC4CCCCC4N4CC(c5cncc(C(F)(F)F)c5)C4)ccc3C2=O)C(=O)N1] | 3-(1-oxo-5-((2-(3-(5- (trifluoromethyl)pyridin-3- yl)azetidin-1- yl)cyclohexyl)oxy)isoindolin-2- yl)piperidine-2,6-dione |
| 179-B | 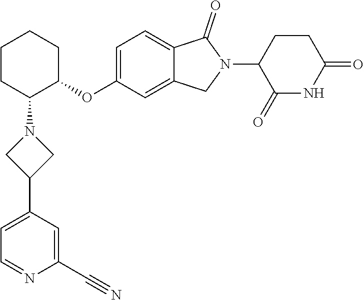 [SMILES: N#Cc1cc(C2CN([C@@H]3CCCC[C@@H]3Oc3ccc4c(c3)CN(C3CCC(=O)NC3=O)C4=O)C2)ccn1] | Rac-4-(1-((cis)-2-((2-(2,6- dioxopiperidin-3-yl)-1- oxoisoindolin-5- yl)oxy)cyclohexyl)azetidin-3- yl)picolinonitrile |
| 180-B | 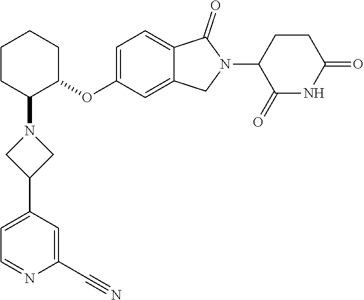 [SMILES: N#Cc1cc(C2CN([C@H]3CCCC[C@@H]3Oc3ccc4c(c3)CN(C3CCC(=O)NC3=O)C4=O)C2)ccn1] | Rac-4-(1-((trans)-2-((2-(2,6- dioxopiperidin-3-yl)-1- oxoisoindolin-5- yl)oxy)cyclohexyl)azetidin-3- yl)picolinonitrile |
| 181-B | 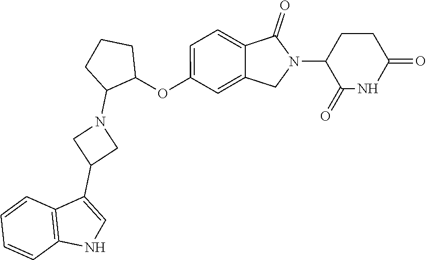 [SMILES: O=C1CCC(N2Cc3cc(OC4CCCC4N4CC(c5c[nH]c6ccccc56)C4)ccc3C2=O)C(=O)N1] | 3-(5-((2-(3-(1H-indol-3-yl)azetidin- 1-yl)cyclopentyl)oxy)-1- oxoisoindolin-2-yl)piperidine-2,6- dione |
| 182-B | 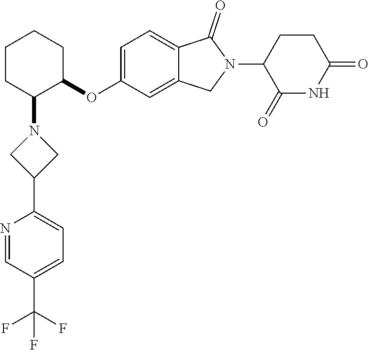 [SMILES: O=C1CCC(N2Cc3cc(O[C@@H]4CCCC[C@@H]4N4CC(c5ccc(C(F)(F)F)cn5)C4)ccc3C2=O)C(=O)N1] | Rac-3-(1-oxo-5-(((cis)-2-(3-(5- (trifluoromethyl)pyridin-2- yl)azetidin-1- yl)cyclohexyl)oxy)isoindolin-2- yl)piperidine-2,6-dione |
| 183-B | 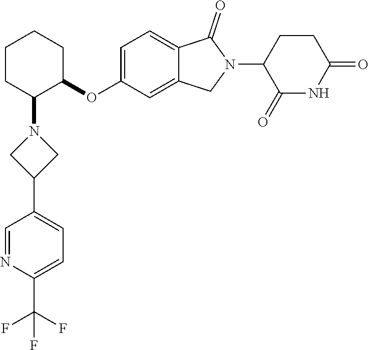 [SMILES: O=C1CCC(N2Cc3cc(O[C@@H]4CCCC[C@@H]4N4CC(c5ccc(C(F)(F)F)nc5)C4)ccc3C2=O)C(=O)N1] | Rac-3-(1-oxo-5-(((cis)-2-(3-(6- (trifluoromethyl)pyridin-3- yl)azetidin-1- yl)cyclohexyl)oxy)isoindolin-2- yl)piperidine-2,6-dione |
| 184-B | 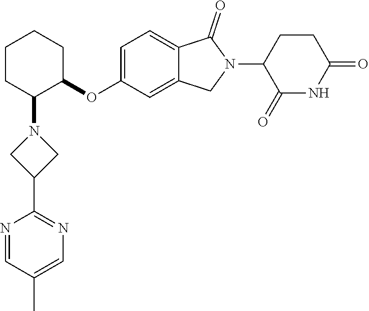 [SMILES: Cc1cnc(C2CN([C@H]3CCCC[C@H]3Oc3ccc4c(c3)CN(C3CCC(=O)NC3=O)C4=O)C2)nc1] | Rac-3-(5-(((cis)-2-(3-(5- methylpyrimidin-2-yl)azetidin-1- yl)cyclohexyl)oxy)-1- oxoisoindolin-2-yl)piperidine-2,6- dione |
| 185-B | 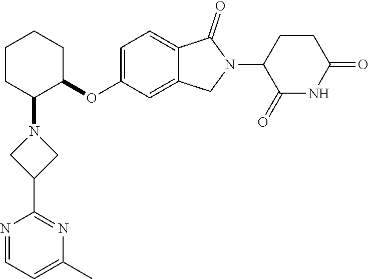 [SMILES: Cc1ccnc(C2CN([C@H]3CCCC[C@H]3Oc3ccc4c(c3)CN(C3CCC(=O)NC3=O)C4=O)C2)n1] | Rac-3-(5-(((cis)-2-(3-(4- methylpyrimidin-2-yl)azetidin-1- yl)cyclohexyl)oxy)-1- oxoisoindolin-2-yl)piperidine-2,6- dione |
| 186-B | 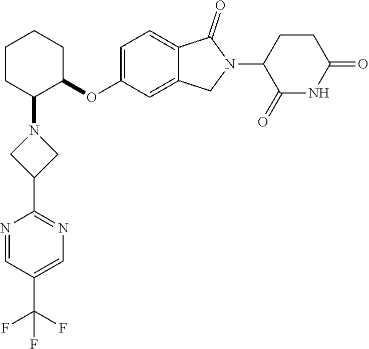 [SMILES: O=C1CCC(N2Cc3cc(O[C@@H]4CCCC[C@@H]4N4CC(c5ncc(C(F)(F)F)cn5)C4)ccc3C2=O)C(=O)N1] | Rac-3-(1-oxo-5-(((cis)-2-(3-(5- (trifluoromethyl)pyrimidin-2- yl)azetidin-1- yl)cyclohexyl)oxy)isoindolin-2- yl)piperidine-2,6-dione |
| 187-B | 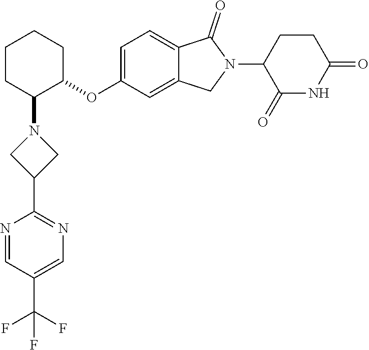 [SMILES: O=C1CCC(N2Cc3cc(O[C@H]4CCCC[C@@H]4N4CC(c5ncc(C(F)(F)F)cn5)C4)ccc3C2=O)C(=O)N1] | Rac-3-(1-oxo-5-(((trans)-2-(3-(5- (trifluoromethyl)pyrimidin-2- yl)azetidin-1- yl)cyclohexyl)oxy)isoindolin-2- yl)piperidine-2,6-dione |
| 188-B | 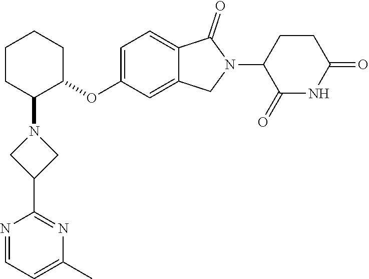 [SMILES: Cc1ccnc(C2CN([C@H]3CCCC[C@@H]3Oc3ccc4c(c3)CN(C3CCC(=O)NC3=O)C4=O)C2)n1] | Rac-3-(5-(((trans)-2-(3-(4- methylpyrimidin-2-yl)azetidin-1- yl)cyclohexyl)oxy)-1- oxoisoindolin-2-yl)piperidine-2,6- dione |
| 189-B | 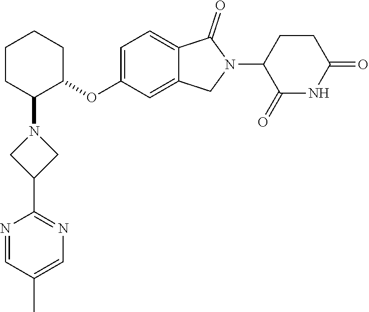 [SMILES: Cc1cnc(C2CN([C@H]3CCCC[C@@H]3Oc3ccc4c(c3)CN(C3CCC(=O)NC3=O)C4=O)C2)nc1] | Rac-3-(5-(((trans)-2-(3-(5- methylpyrimidin-2-yl)azetidin-1- yl)cyclohexyl)oxy)-1- oxoisoindolin-2-yl)piperidine-2,6- dione |
| 190-B | 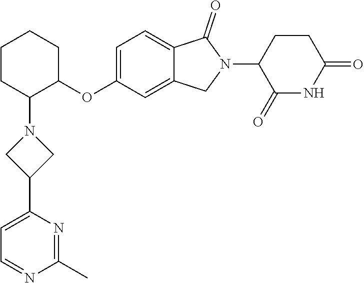 [SMILES: Cc1nccc(C2CN(C3CCCCC3Oc3ccc4c(c3)CN(C3CCC(=O)NC3=O)C4=O)C2)n1] | 3-(5-((2-(3-(2-methylpyrimidin-4- yl)azetidin-1-yl)cyclohexyl)oxy)-1- oxoisoindolin-2-yl)piperidine-2,6- dione |
| 191-B | 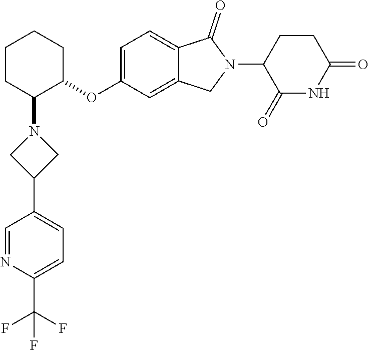 [SMILES: O=C1CCC(N2Cc3cc(O[C@H]4CCCC[C@@H]4N4CC(c5ccc(C(F)(F)F)nc5)C4)ccc3C2=O)C(=O)N1] | Rac-3-(1-oxo-5-(((trans)-2-(3-(6- (trifluoromethyl)pyridin-3- yl)azetidin-1- yl)cyclohexyl)oxy)isoindolin-2- yl)piperidine-2,6-dione |
| 192-B | 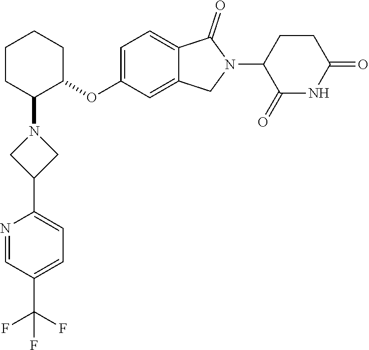 [SMILES: O=C1CCC(N2Cc3cc(O[C@H]4CCCC[C@@H]4N4CC(c5ccc(C(F)(F)F)cn5)C4)ccc3C2=O)C(=O)N1] | Rac-3-(1-oxo-5-(((trans)-2-(3-(5- (trifluoromethyl)pyridin-2- yl)azetidin-1- yl)cyclohexyl)oxy)isoindolin-2- yl)piperidine-2,6-dione |
| 193-B | 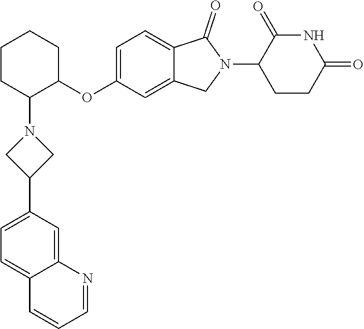 [SMILES: O=C1CCC(N2Cc3cc(OC4CCCCC4N4CC(c5ccc6cccnc6c5)C4)ccc3C2=O)C(=O)N1] | 3-(1-oxo-5-((2-(3-(quinolin-7- yl)azetidin-1- yl)cyclohexyl)oxy)isoindolin-2- yl)piperidine-2,6-dione |
| 194-B | 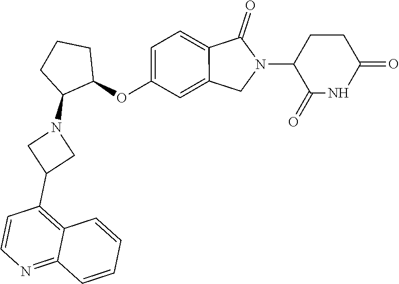 [SMILES: O=C1CCC(N2Cc3cc(O[C@@H]4CCC[C@@H]4N4CC(c5ccnc6ccccc56)C4)ccc3C2=O)C(=O)N1] | 3-(1-oxo-5-(((1R,2S)-2-(3- (quinolin-4-yl)azetidin-1- yl)cyclopentyl)oxy)isoindolin-2- yl)piperidine-2,6-dione |
| 195-B | 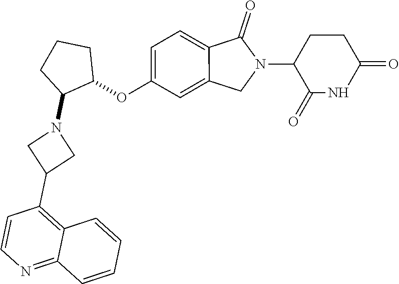 | Rac-3-(1-oxo-5-(((trans)-2-(3- (quinolin-4-yl)azetidin-1- yl)cyclopentyl)oxy)isoindolin-2- yl)piperidine-2,6-dione |
| 196-B | 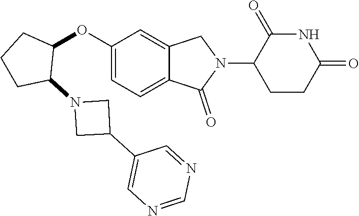 | Rac-3-(1-oxo-5-(((cis)-2-(3- (pyrimidin-5-yl)azetidin-1- yl)cyclopentyl)oxy)isoindolin-2- yl)piperidine-2,6-dione |
| 197-B | 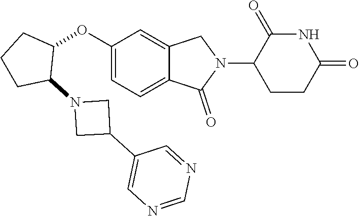 | Rac-3-(1-oxo-5-(((trans)-2-(3- (pyrimidin-5-yl)azetidin-1- yl)cyclopentyl)oxy)isoindolin-2- yl)piperidine-2,6-dione |
| 198-B | 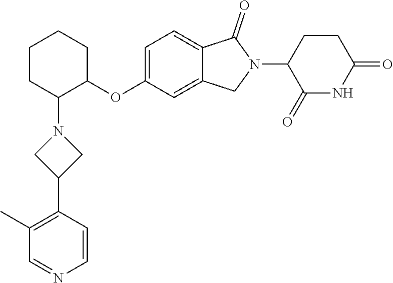 [SMILES: Cc1cnccc1C1CN(C2CCCCC2Oc2ccc3c(c2)CN(C2CCC(=O)NC2=O)C3=O)C1] | 3-(5-((2-(3-(3-methylpyridin-4- yl)azetidin-1-yl)cyclohexyl)oxy)-1- oxoisoindolin-2-yl)piperidine-2,6- dione |
| 199-B | 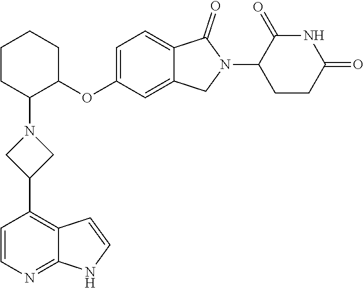 [SMILES: O=C1CCC(N2Cc3cc(OC4CCCCC4N4CC(c5ccnc6[nH]ccc56)C4)ccc3C2=O)C(=O)N1] | 3-(5-((2-(3-(1H-pyrrolo[2,3- b]pyridin-4-yl)azetidin-1- yl)cyclohexyl)oxy)-1- oxoisoindolin-2-yl)piperidine-2,6- dione |
| 200-B | 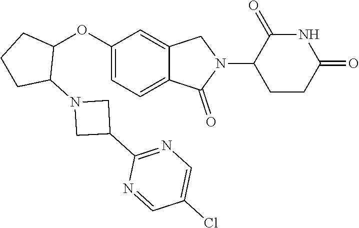 [SMILES: O=C1CCC(N2Cc3cc(OC4CCCC4N4CC(c5ncc(Cl)cn5)C4)ccc3C2=O)C(=O)N1] | 3-(5-((2-(3-(5-chloropyrimidin-2- yl)azetidin-1-yl)cyclopentyl)oxy)-1- oxoisoindolin-2-yl)piperidine-2,6- dione |
| 201-B | 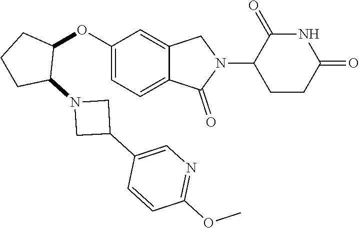 | Rac-3-(5-(((cis)-2-(3-(6- methoxypyridin-3-yl)azetidin-1- yl)cyclopentyl)oxy)-1- oxoisoindolin-2-yl)piperidine-2,6- dione |
| 202-B | 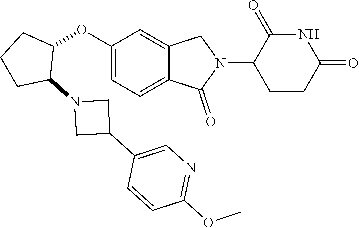 | Rac-3-(5-(((trans)-2-(3-(6- methoxypyridin-3-yl)azetidin-1- yl)cyclopentyl)oxy)-1- oxoisoindolin-2-yl)piperidine-2,6- dione |
| 203-B | 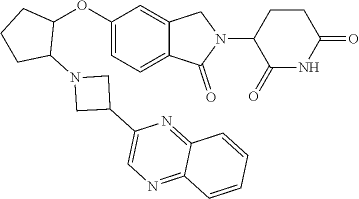 [SMILES: O=C1CCC(N2Cc3cc(OC4CCCC4N4CC(c5cnc6ccccc6n5)C4)ccc3C2=O)C(=O)N1] | 3-(1-oxo-5-((2-(3-(quinoxalin-2- yl)azetidin-1- yl)cyclopentyl)oxy)isoindolin-2- yl)piperidine-2,6-dione |
| 204-B | 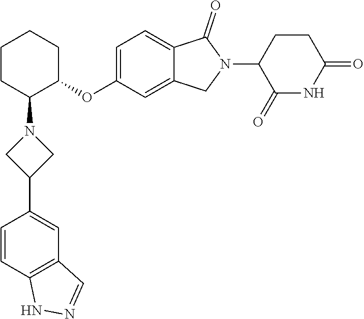 [SMILES: O=C1CCC(N2Cc3cc(O[C@H]4CCCC[C@@H]4N4CC(c5ccc6[nH]ncc6c5)C4)ccc3C2=O)C(=O)N1] | 3-(5-(((1S,2S)-2-(3-(1H-indazol-5- yl)azetidin-1-yl)cyclohexyl)oxy)-1- oxoisoindolin-2-yl)piperidine-2,6- dione |
| 205-B | 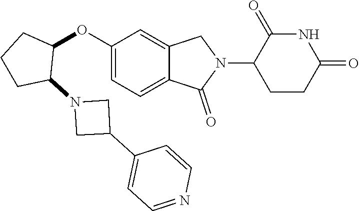 | Rac-3-(1-oxo-5-(((cis)-2-(3- (pyridin-4-yl)azetidin-1- yl)cyclopentyl)oxy)isoindolin-2- yl)piperidine-2,6-dione |
| 206-B | 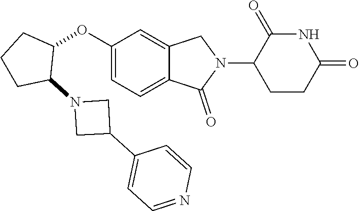 | Rac-3-(1-oxo-5-(((trans)-2-(3- (pyridin-4-yl)azetidin-1- yl)cyclopentyl)oxy)isoindolin-2- yl)piperidine-2,6-dione |
| 207-B | 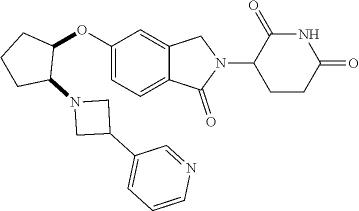 | Rac-3-(1-oxo-5-(((cis)-2-(3- (pyridin-3-yl)azetidin-1- yl)cyclopentyl)oxy)isoindolin-2- yl)piperidine-2,6-dione |
| 208-B | 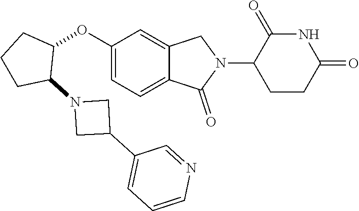 | Rac-3-(1-oxo-5-(((trans)-2-(3- (pyridin-3-yl)azetidin-1- yl)cyclopentyl)oxy)isoindolin-2- yl)piperidine-2,6-dione |
| 209-B | 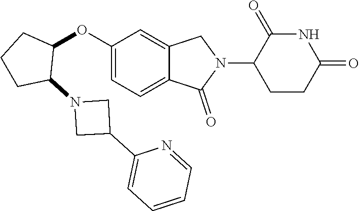 | Rac-3-(1-oxo-5-(((cis)-2-(3- (pyridin-2-yl)azetidin-1- yl)cyclopentyl)oxy)isoindolin-2- yl)piperidine-2,6-dione |
| 210-B | 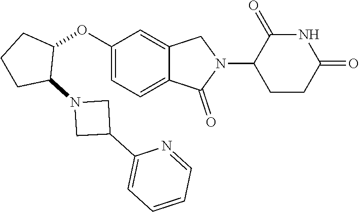 | Rac-3-(1-oxo-5-(((trans)-2-(3- (pyridin-2-yl)azetidin-1- yl)cyclopentyl)oxy)isoindolin-2- yl)piperidine-2,6-dione |
| 211-B | 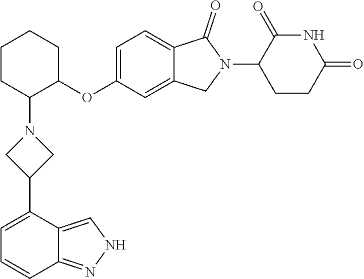 [SMILES: O=C1CCC(N2Cc3cc(OC4CCCCC4N4CC(c5cccc6n[nH]cc56)C4)ccc3C2=O)C(=O)N1] | 3-(5-((2-(3-(2H-indazol-4- yl)azetidin-1-yl)cyclohexyl)oxy)-1- oxoisoindolin-2-yl)piperidine-2,6- dione |
| 212-B | 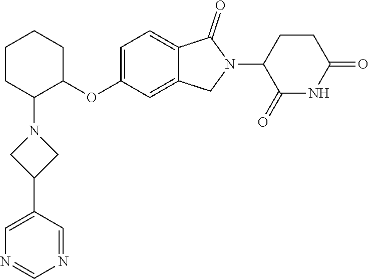 [SMILES: O=C1CCC(N2Cc3cc(OC4CCCCC4N4CC(c5cncnc5)C4)ccc3C2=O)C(=O)N1] | 3-(1-oxo-5-((2-(3-(pyrimidin-5- yl)azetidin-1- yl)cyclohexyl)oxy)isoindolin-2- yl)piperidine-2,6-dione |

In some embodiments, provided herein is a compound which binds cereblon selected from Table 1A, or a pharmaceutically acceptable salt, solvate, stereoisomer, and/or tautomer thereof.

TABLE 1A
Structure

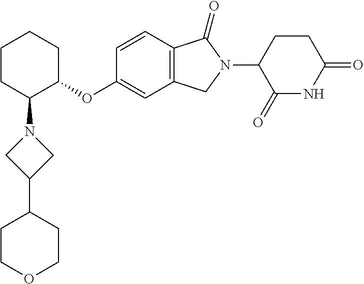 [SMILES: O=C1CCC(N2Cc3cc(O[C@H]4CCCC[C@@H]4N4CC(C5CCOCC5)C4)ccc3C2=O)C(=O)N1]

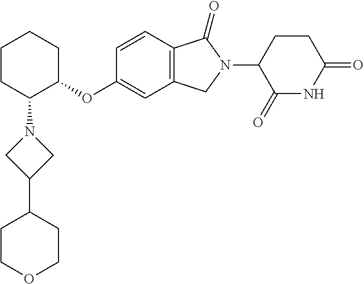 [SMILES: O=C1CCC(N2Cc3cc(O[C@H]4CCCC[C@H]4N4CC(C5CCOCC5)C4)ccc3C2=O)C(=O)N1]

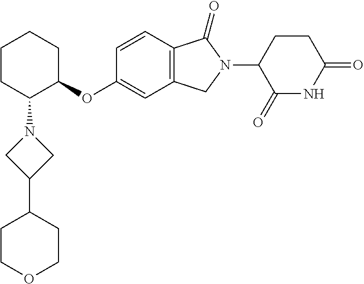 [SMILES: O=C1CCC(N2Cc3cc(O[C@@H]4CCCC[C@H]4N4CC(C5CCOCC5)C4)ccc3C2=O)C(=O)N1]

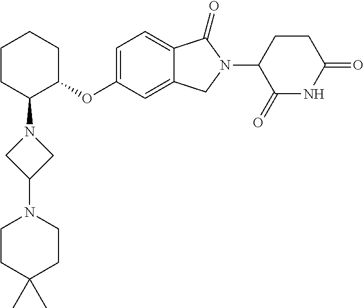 [SMILES: CC1(C)CCN(C2CN([C@H]3CCCC[C@@H]3Oc3ccc4c(c3)CN(C3CCC(=O)NC3=O)C4=O)C2)CC1]

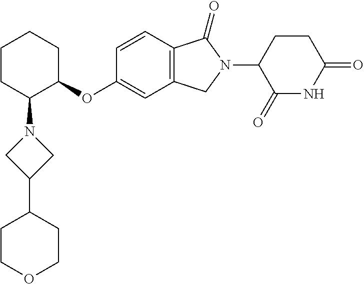 [SMILES: O=C1CCC(N2Cc3cc(O[C@@H]4CCCC[C@@H]4N4CC(C5CCOCC5)C4)ccc3C2=O)C(=O)N1]

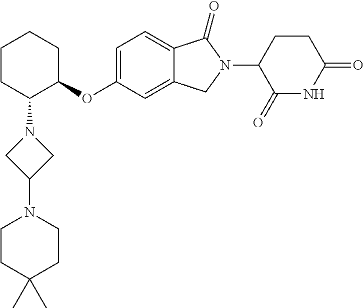 [SMILES: CC1(C)CCN(C2CN([C@@H]3CCCC[C@H]3Oc3ccc4c(c3)CN(C3CCC(=O)NC3=O)C4=O)C2)CC1]

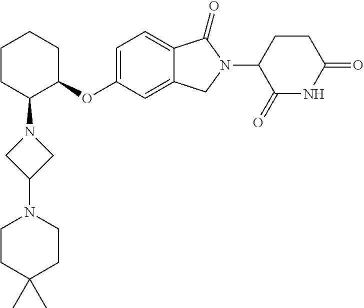 [SMILES: CC1(C)CCN(C2CN([C@H]3CCCC[C@H]3Oc3ccc4c(c3)CN(C3CCC(=O)NC3=O)C4=O)C2)CC1]

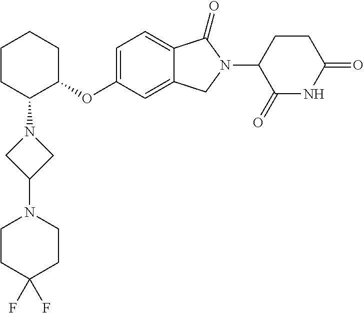 [SMILES: O=C1CCC(N2Cc3cc(O[C@H]4CCCC[C@H]4N4CC(N5CCC(F)(F)CC5)C4)ccc3C2=O)C(=O)N1]

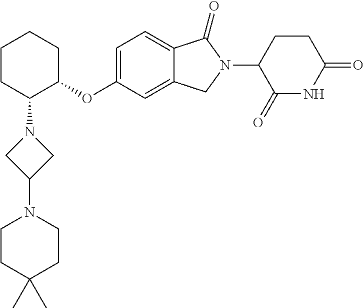 [SMILES: CC1(C)CCN(C2CN([C@@H]3CCCC[C@@H]3Oc3ccc4c(c3)CN(C3CCC(=O)NC3=O)C4=O)C2)CC1]

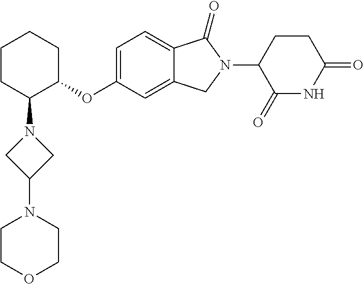 [SMILES: O=C1CCC(N2Cc3cc(O[C@H]4CCCC[C@@H]4N4CC(N5CCOCC5)C4)ccc3C2=O)C(=O)N1]

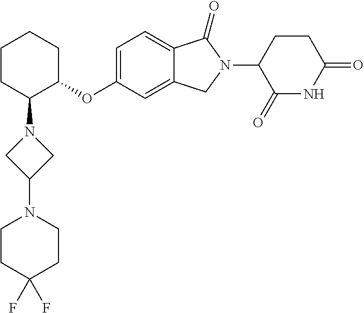 [SMILES: O=C1CCC(N2Cc3cc(O[C@H]4CCCC[C@@H]4N4CC(N5CCC(F)(F)CC5)C4)ccc3C2=O)C(=O)N1]

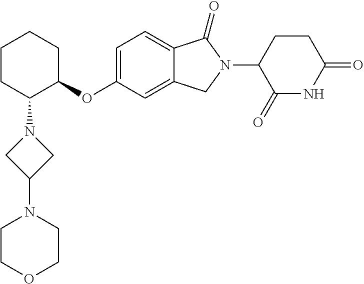 [SMILES: O=C1CCC(N2Cc3cc(O[C@@H]4CCCC[C@H]4N4CC(N5CCOCC5)C4)ccc3C2=O)C(=O)N1]

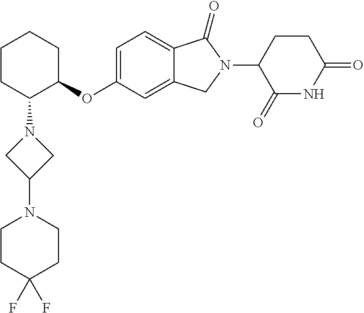 [SMILES: O=C1CCC(N2Cc3cc(O[C@@H]4CCCC[C@H]4N4CC(N5CCC(F)(F)CC5)C4)ccc3C2=O)C(=O)N1]

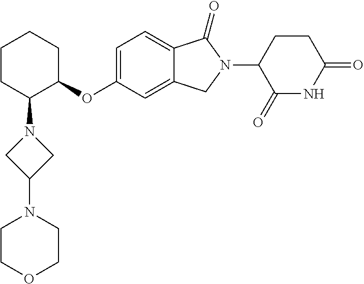 [SMILES: O=C1CCC(N2Cc3cc(O[C@@H]4CCCC[C@@H]4N4CC(N5CCOCC5)C4)ccc3C2=O)C(=O)N1]

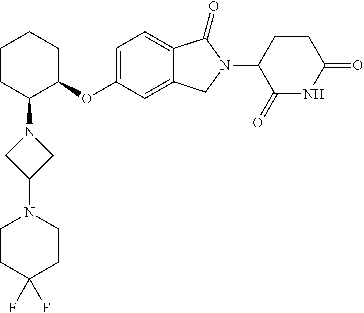 [SMILES: O=C1CCC(N2Cc3cc(O[C@@H]4CCCC[C@@H]4N4CC(N5CCC(F)(F)CC5)C4)ccc3C2=O)C(=O)N1]

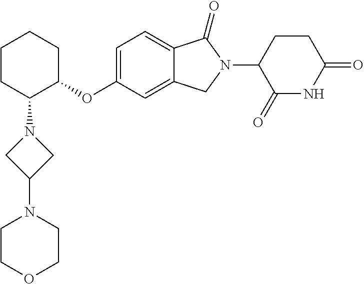 [SMILES: O=C1CCC(N2Cc3cc(O[C@H]4CCCC[C@H]4N4CC(N5CCOCC5)C4)ccc3C2=O)C(=O)N1]

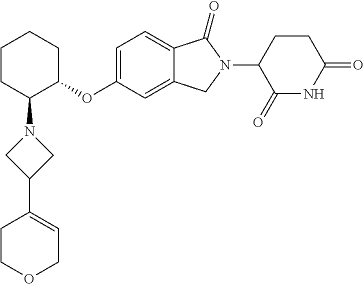 [SMILES: O=C1CCC(N2Cc3cc(O[C@H]4CCCC[C@@H]4N4CC(C5=CCOCC5)C4)ccc3C2=O)C(=O)N1]

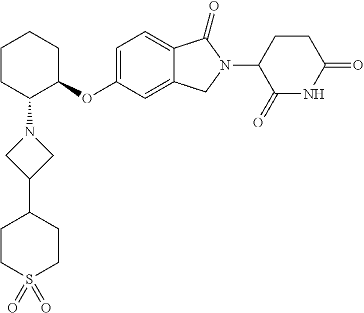 [SMILES: O=C1CCC(N2Cc3cc(O[C@@H]4CCCC[C@H]4N4CC(C5CCS(=O)(=O)CC5)C4)ccc3C2=O)C(=O)N1]

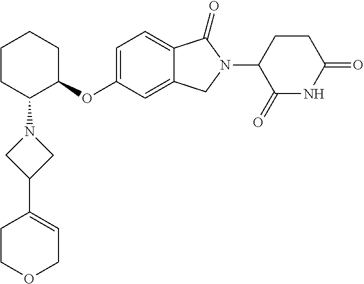 [SMILES: O=C1CCC(N2Cc3cc(O[C@@H]4CCCC[C@H]4N4CC(C5=CCOCC5)C4)ccc3C2=O)C(=O)N1]

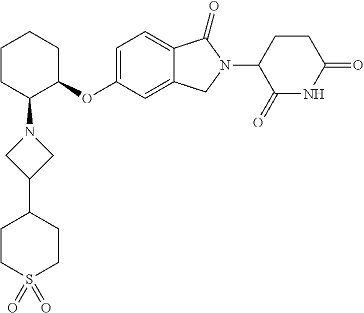 [SMILES: O=C1CCC(N2Cc3cc(O[C@@H]4CCCC[C@@H]4N4CC(C5CCS(=O)(=O)CC5)C4)ccc3C2=O)C(=O)N1]

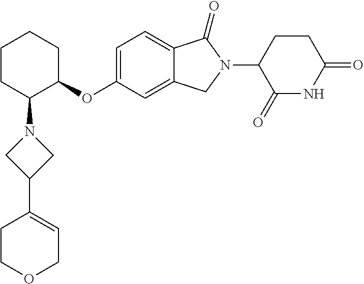 [SMILES: O=C1CCC(N2Cc3cc(O[C@@H]4CCCC[C@@H]4N4CC(C5=CCOCC5)C4)ccc3C2=O)C(=O)N1]

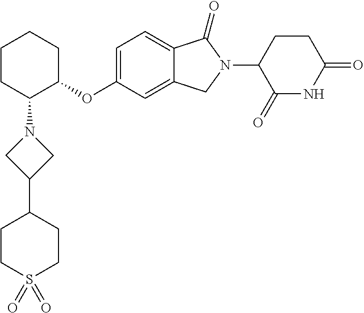 [SMILES: O=C1CCC(N2Cc3cc(O[C@H]4CCCC[C@H]4N4CC(C5CCS(=O)(=O)CC5)C4)ccc3C2=O)C(=O)N1]

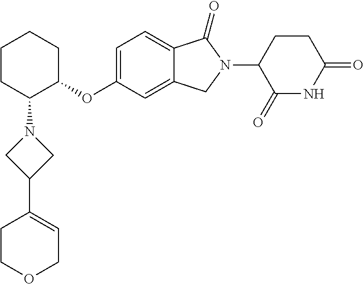 [SMILES: O=C1CCC(N2Cc3cc(O[C@H]4CCCC[C@H]4N4CC(C5=CCOCC5)C4)ccc3C2=O)C(=O)N1]

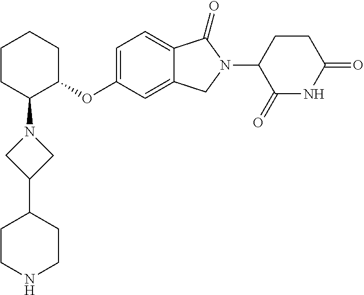 [SMILES: O=C1CCC(N2Cc3cc(O[C@H]4CCCC[C@@H]4N4CC(C5CCNCC5)C4)ccc3C2=O)C(=O)N1]

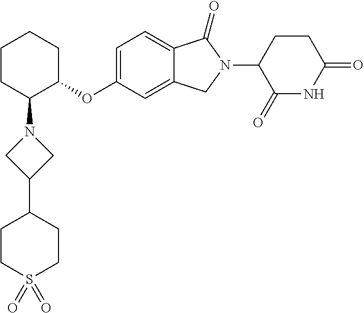 [SMILES: O=C1CCC(N2Cc3cc(O[C@H]4CCCC[C@@H]4N4CC(C5CCS(=O)(=O)CC5)C4)ccc3C2=O)C(=O)N1]

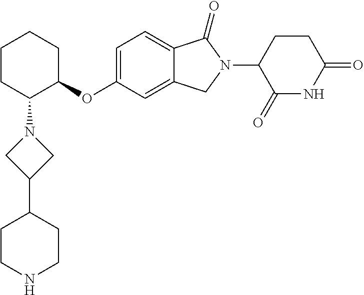 [SMILES: O=C1CCC(N2Cc3cc(O[C@@H]4CCCC[C@H]4N4CC(C5CCNCC5)C4)ccc3C2=O)C(=O)N1]

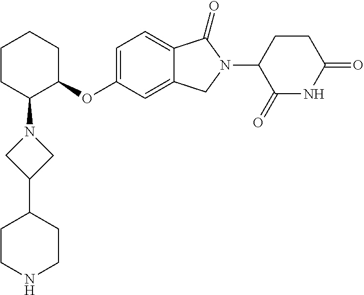 [SMILES: O=C1CCC(N2Cc3cc(O[C@@H]4CCCC[C@@H]4N4CC(C5CCNCC5)C4)ccc3C2=O)C(=O)N1]

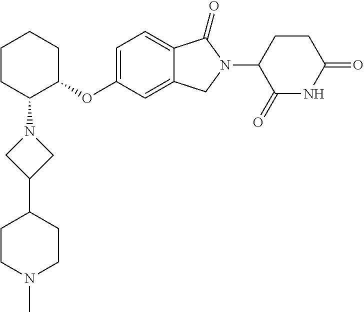 [SMILES: CN1CCC(C2CN([C@@H]3CCCC[C@@H]3Oc3ccc4c(c3)CN(C3CCC(=O)NC3=O)C4=O)C2)CC1]

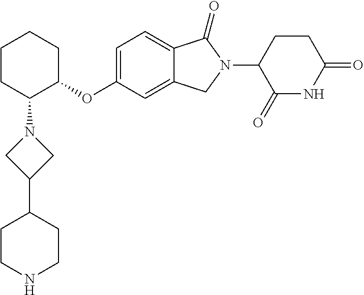 [SMILES: O=C1CCC(N2Cc3cc(O[C@H]4CCCC[C@H]4N4CC(C5CCNCC5)C4)ccc3C2=O)C(=O)N1]

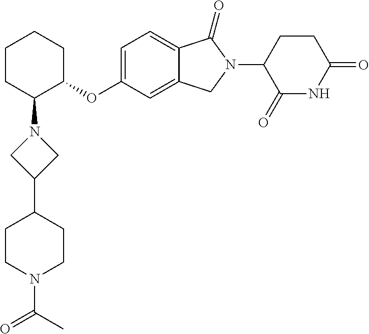 [SMILES: CC(=O)N1CCC(C2CN([C@H]3CCCC[C@@H]3Oc3ccc4c(c3)CN(C3CCC(=O)NC3=O)C4=O)C2)CC1]

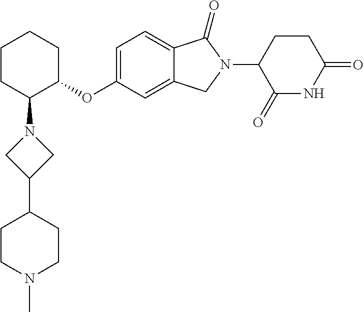 [SMILES: CN1CCC(C2CN([C@H]3CCCC[C@@H]3Oc3ccc4c(c3)CN(C3CCC(=O)NC3=O)C4=O)C2)CC1]

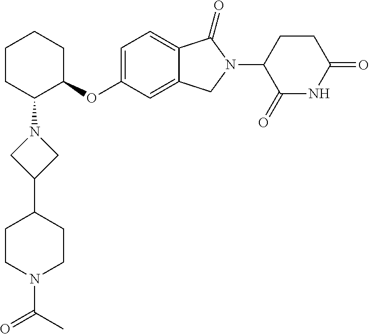 [SMILES: CC(=O)N1CCC(C2CN([C@@H]3CCCC[C@H]3Oc3ccc4c(c3)CN(C3CCC(=O)NC3=O)C4=O)C2)CC1]

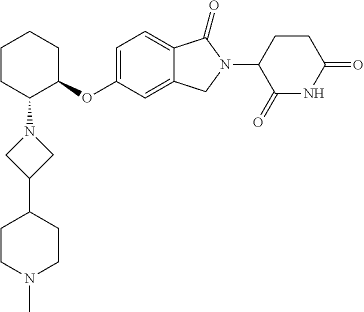 [SMILES: CN1CCC(C2CN([C@@H]3CCCC[C@H]3Oc3ccc4c(c3)CN(C3CCC(=O)NC3=O)C4=O)C2)CC1]

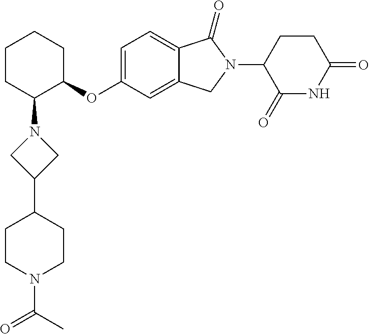 [SMILES: CC(=O)N1CCC(C2CN([C@H]3CCCC[C@H]3Oc3ccc4c(c3)CN(C3CCC(=O)NC3=O)C4=O)C2)CC1]

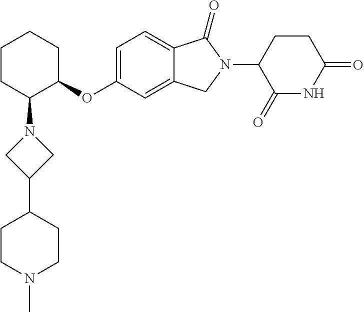 [SMILES: CN1CCC(C2CN([C@H]3CCCC[C@H]3Oc3ccc4c(c3)CN(C3CCC(=O)NC3=O)C4=O)C2)CC1]

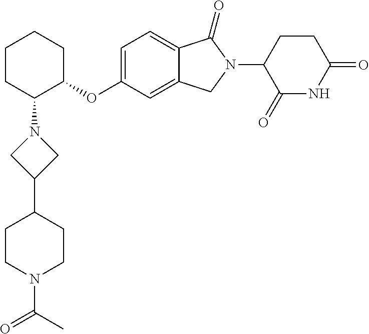 [SMILES: CC(=O)N1CCC(C2CN([C@@H]3CCCC[C@@H]3Oc3ccc4c(c3)CN(C3CCC(=O)NC3=O)C4=O)C2)CC1]

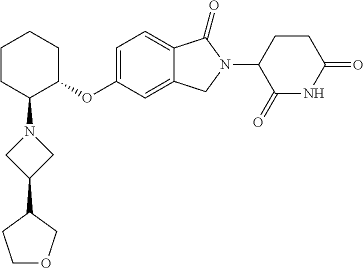

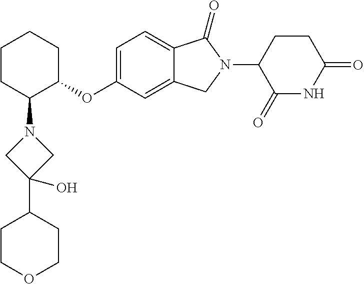 [SMILES: O=C1CCC(N2Cc3cc(O[C@H]4CCCC[C@@H]4N4CC(O)(C5CCOCC5)C4)ccc3C2=O)C(=O)N1]

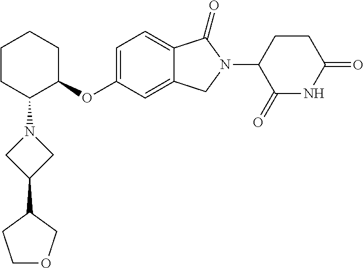

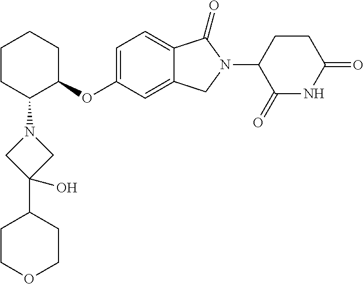 [SMILES: O=C1CCC(N2Cc3cc(O[C@@H]4CCCC[C@H]4N4CC(O)(C5CCOCC5)C4)ccc3C2=O)C(=O)N1]

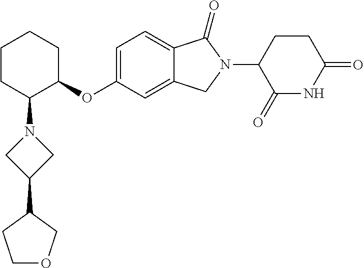

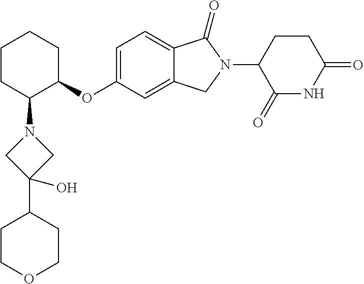 [SMILES: O=C1CCC(N2Cc3cc(O[C@@H]4CCCC[C@@H]4N4CC(O)(C5CCOCC5)C4)ccc3C2=O)C(=O)N1]

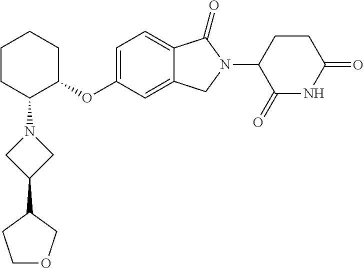

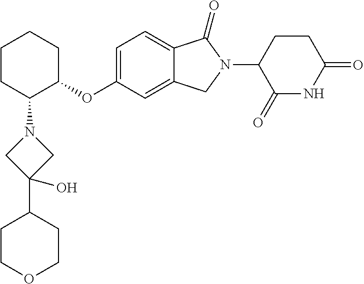 [SMILES: O=C1CCC(N2Cc3cc(O[C@H]4CCCC[C@H]4N4CC(O)(C5CCOCC5)C4)ccc3C2=O)C(=O)N1]

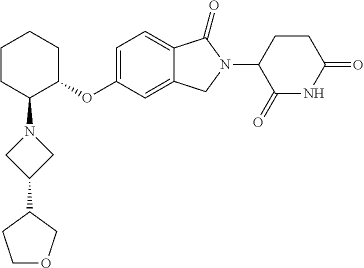

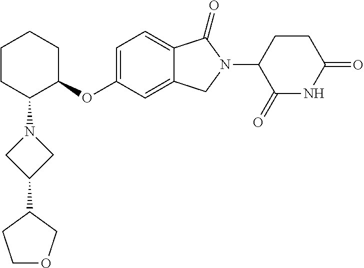

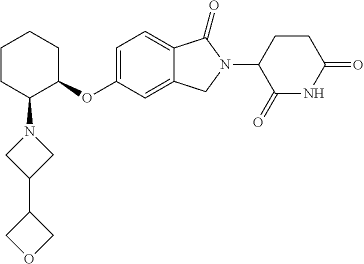 [SMILES: O=C1CCC(N2Cc3cc(O[C@@H]4CCCC[C@@H]4N4CC(C5COC5)C4)ccc3C2=O)C(=O)N1]

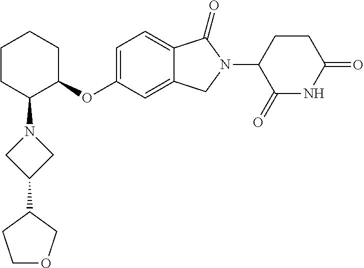

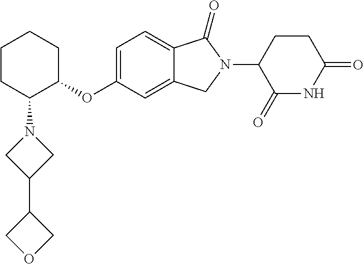 [SMILES: O=C1CCC(N2Cc3cc(O[C@H]4CCCC[C@H]4N4CC(C5COC5)C4)ccc3C2=O)C(=O)N1]

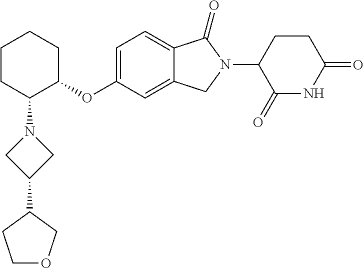

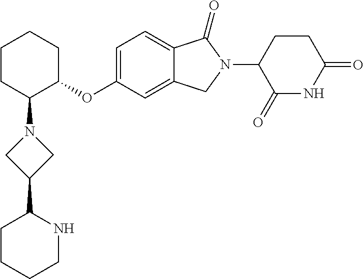

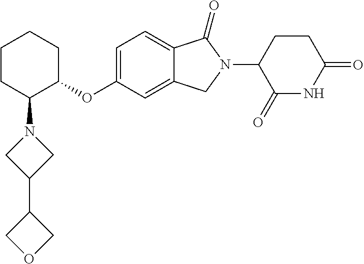 [SMILES: O=C1CCC(N2Cc3cc(O[C@H]4CCCC[C@@H]4N4CC(C5COC5)C4)ccc3C2=O)C(=O)N1]

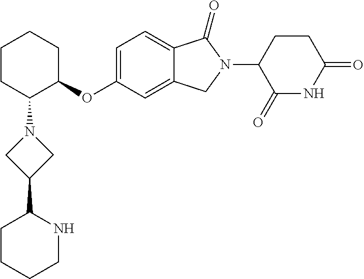

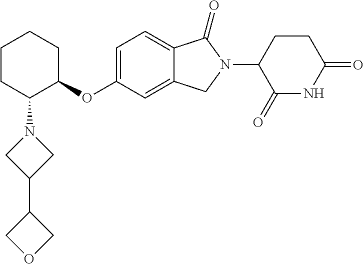 [SMILES: O=C1CCC(N2Cc3cc(O[C@@H]4CCCC[C@H]4N4CC(C5COC5)C4)ccc3C2=O)C(=O)N1]

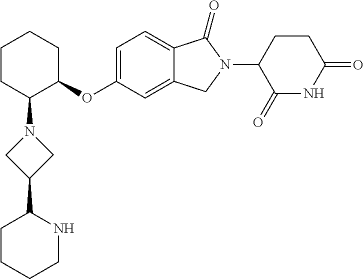

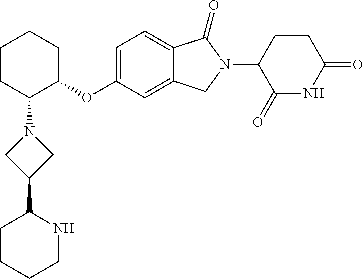

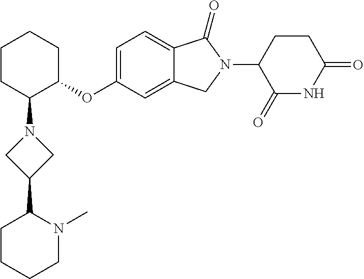

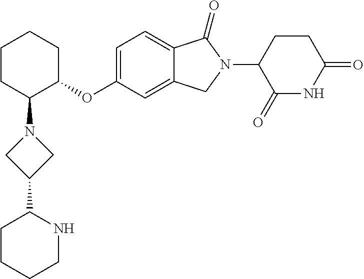

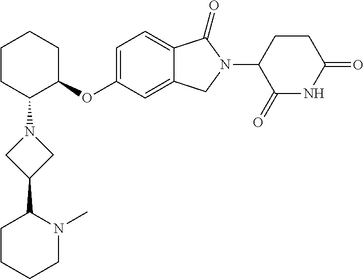

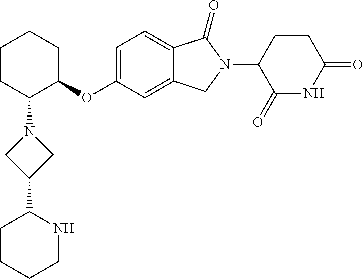

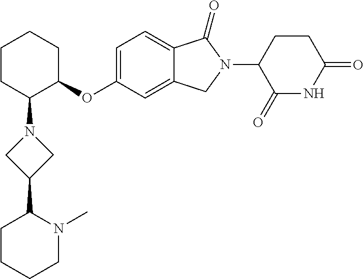

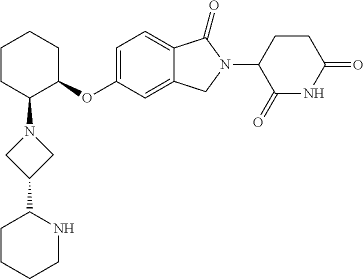

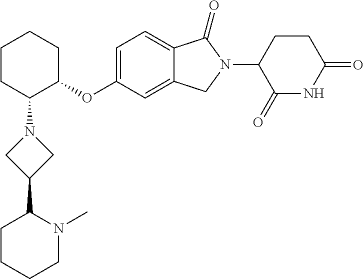

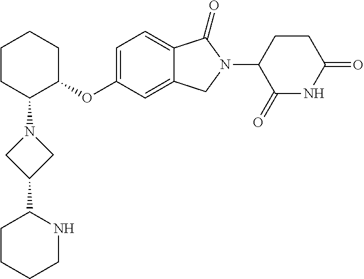

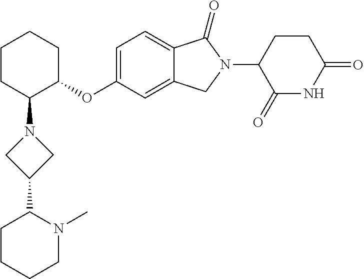

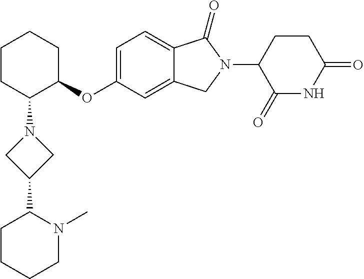

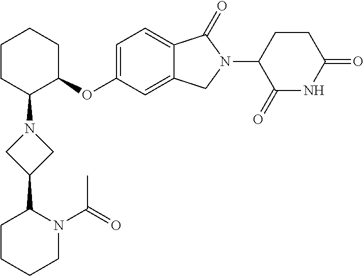

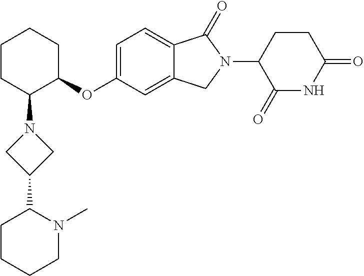

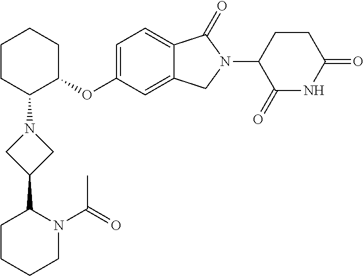

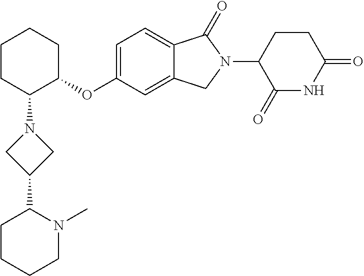

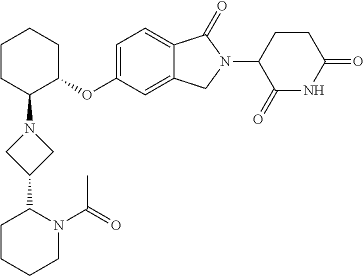

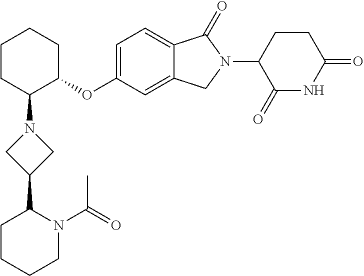

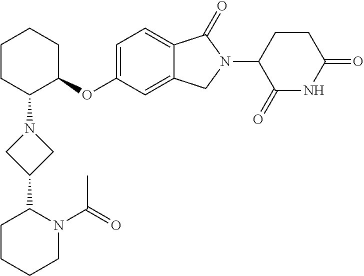

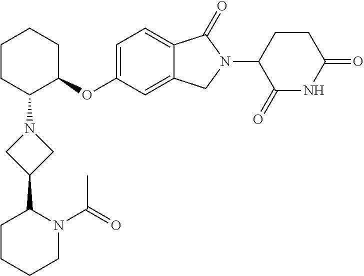

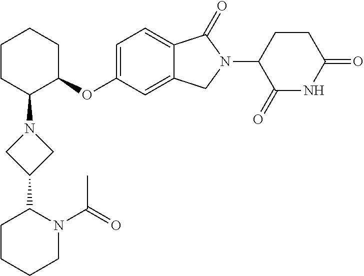

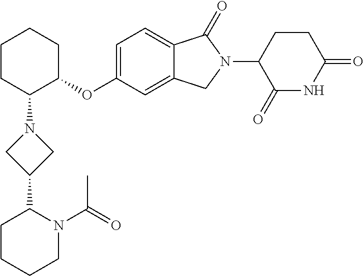

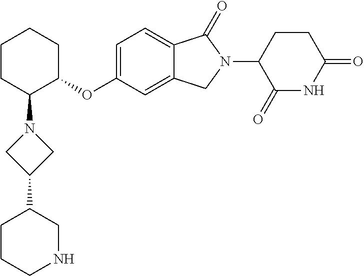

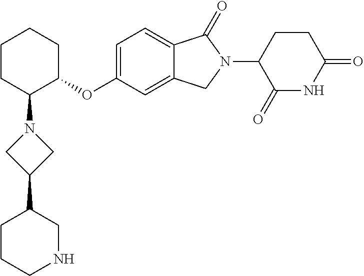

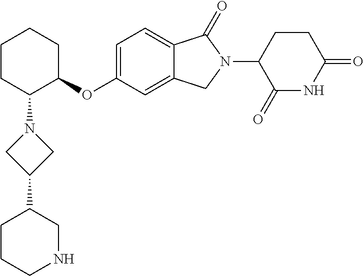

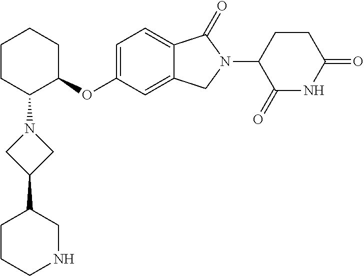

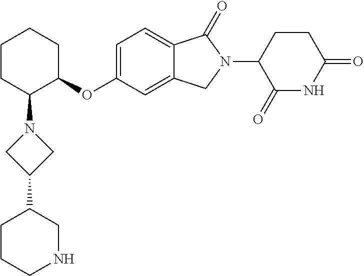

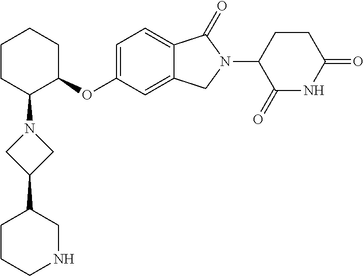

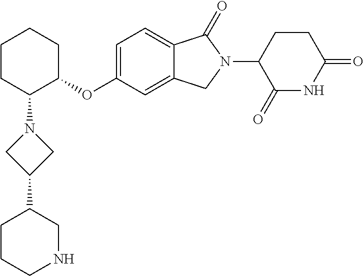

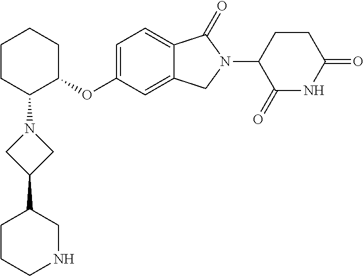

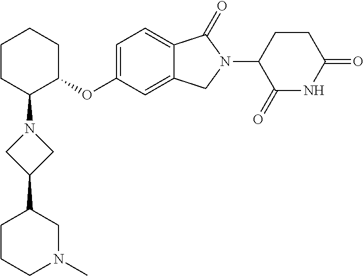

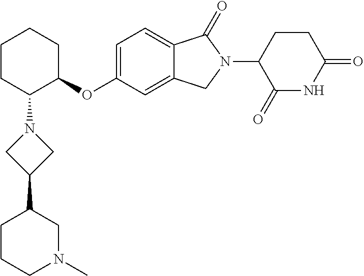

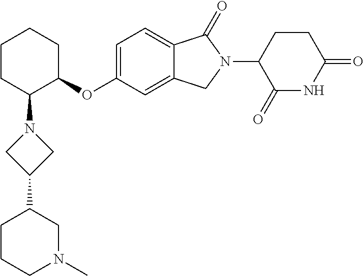

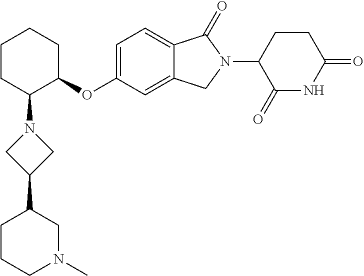

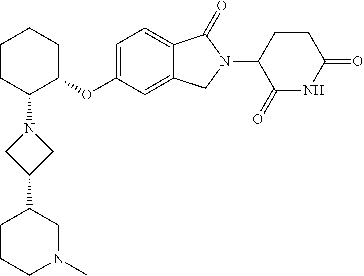

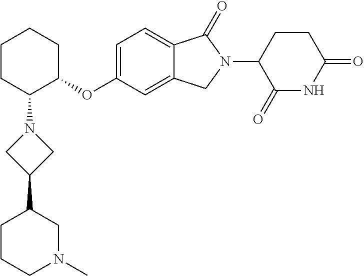

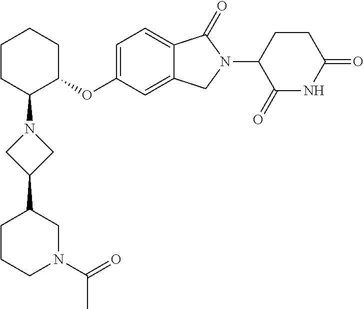

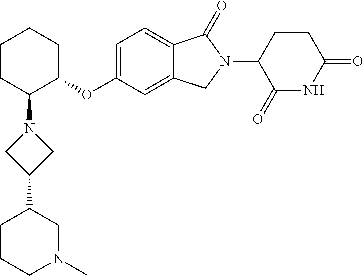

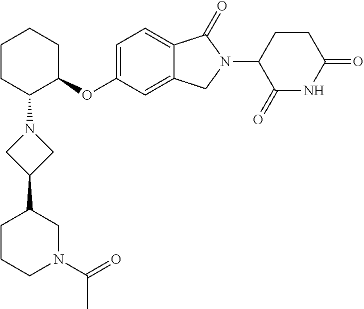

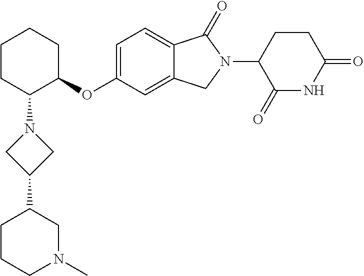

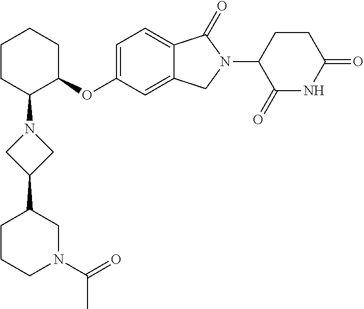

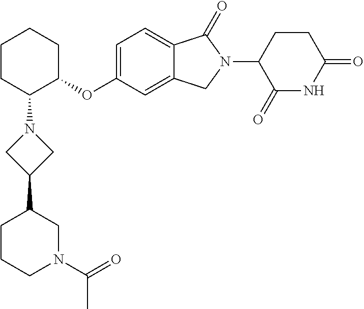

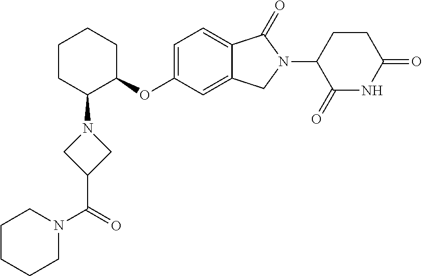 [SMILES: O=C1CCC(N2Cc3cc(O[C@@H]4CCCC[C@@H]4N4CC(C(=O)N5CCCCC5)C4)ccc3C2=O)C(=O)N1]

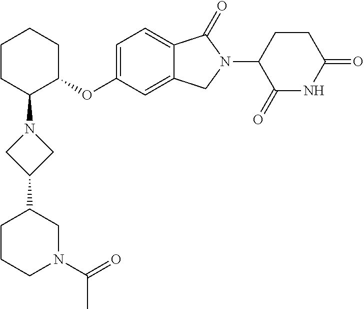

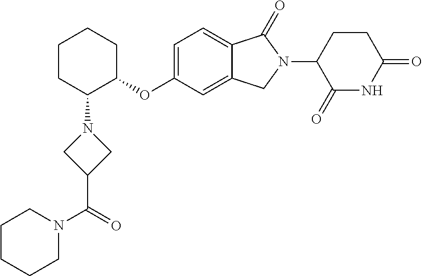 [SMILES: O=C1CCC(N2Cc3cc(O[C@H]4CCCC[C@H]4N4CC(C(=O)N5CCCCC5)C4)ccc3C2=O)C(=O)N1]

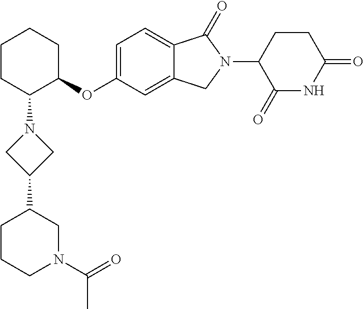

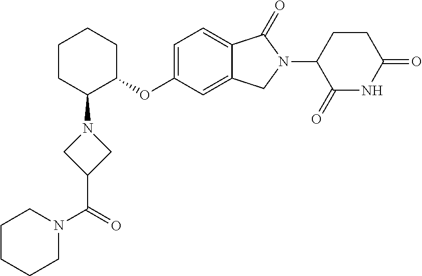 [SMILES: O=C1CCC(N2Cc3cc(O[C@H]4CCCC[C@@H]4N4CC(C(=O)N5CCCCC5)C4)ccc3C2=O)C(=O)N1]

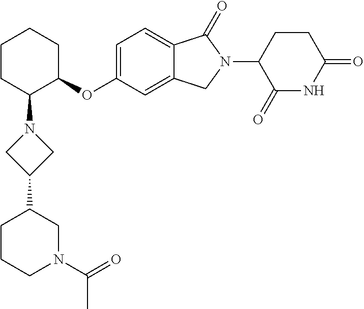

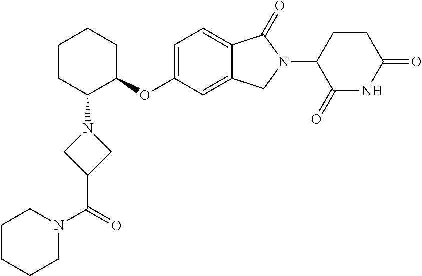 [SMILES: O=C1CCC(N2Cc3cc(O[C@@H]4CCCC[C@H]4N4CC(C(=O)N5CCCCC5)C4)ccc3C2=O)C(=O)N1]

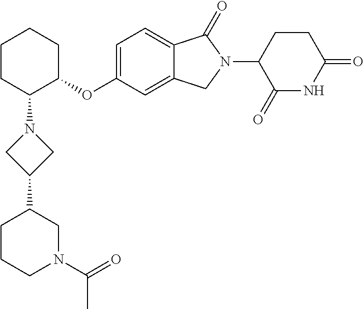

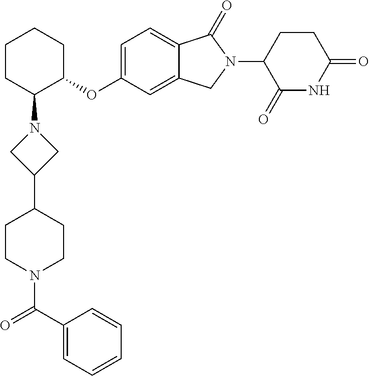 [SMILES: O=C1CCC(N2Cc3cc(O[C@H]4CCCC[C@@H]4N4CC(C5CCN(C(=O)c6ccccc6)CC5)C4)ccc3C2=O)C(=O)N1]

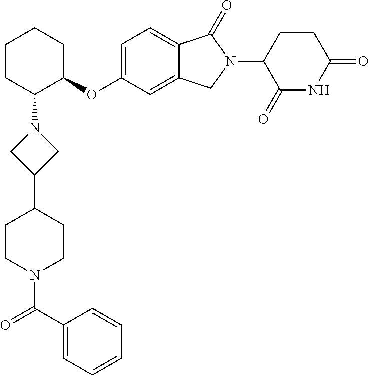 [SMILES: O=C1CCC(N2Cc3cc(O[C@@H]4CCCC[C@H]4N4CC(C5CCN(C(=O)c6ccccc6)CC5)C4)ccc3C2=O)C(=O)N1]

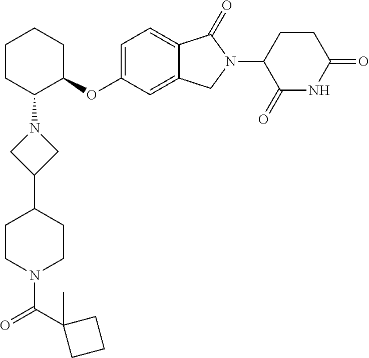 [SMILES: CC1(C(=O)N2CCC(C3CN([C@@H]4CCCC[C@H]4Oc4ccc5c(c4)CN(C4CCC(=O)NC4=O)C5=O)C3)CC2)CCC1]

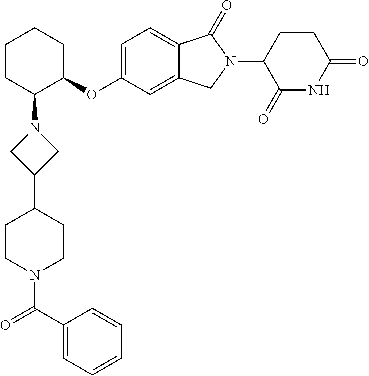 [SMILES: O=C1CCC(N2Cc3cc(O[C@@H]4CCCC[C@@H]4N4CC(C5CCN(C(=O)c6ccccc6)CC5)C4)ccc3C2=O)C(=O)N1]

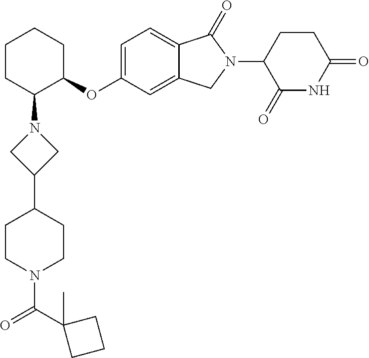 [SMILES: CC1(C(=O)N2CCC(C3CN([C@H]4CCCC[C@H]4Oc4ccc5c(c4)CN(C4CCC(=O)NC4=O)C5=O)C3)CC2)CCC1]

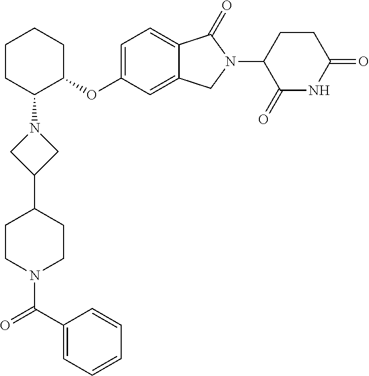 [SMILES: O=C1CCC(N2Cc3cc(O[C@H]4CCCC[C@H]4N4CC(C5CCN(C(=O)c6ccccc6)CC5)C4)ccc3C2=O)C(=O)N1]

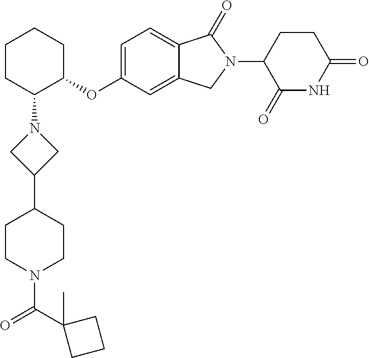 [SMILES: CC1(C(=O)N2CCC(C3CN([C@@H]4CCCC[C@@H]4Oc4ccc5c(c4)CN(C4CCC(=O)NC4=O)C5=O)C3)CC2)CCC1]

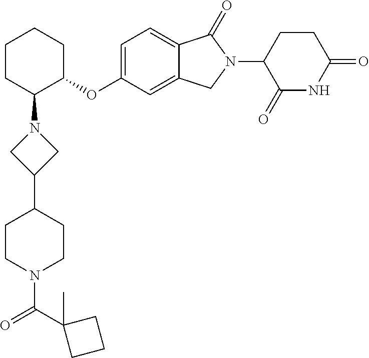 [SMILES: CC1(C(=O)N2CCC(C3CN([C@H]4CCCC[C@@H]4Oc4ccc5c(c4)CN(C4CCC(=O)NC4=O)C5=O)C3)CC2)CCC1]

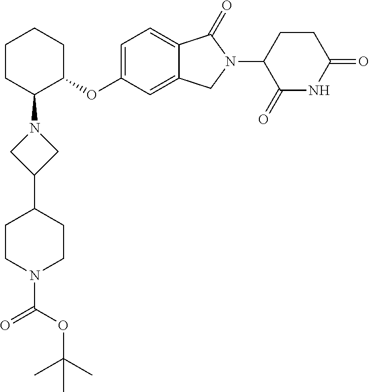 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1]

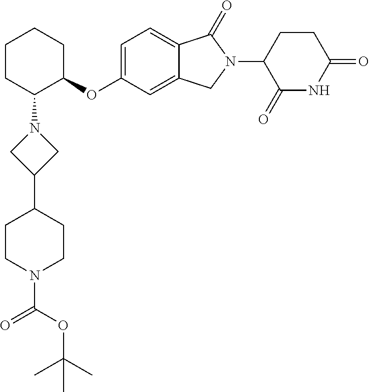 [SMILES: CC(C)(C)OC(=O)N1CCC(C2CN([C@@H]3CCCC[C@H]3Oc3ccc4c(c3)CN(C3CCC(=O)NC3=O)C4=O)C2)CC1]

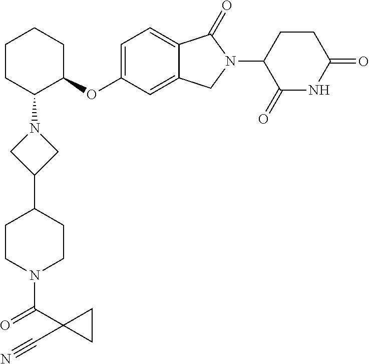 [SMILES: N#CC1(C(=O)N2CCC(C3CN([C@@H]4CCCC[C@H]4Oc4ccc5c(c4)CN(C4CCC(=O)NC4=O)C5=O)C3)CC2)CC1]

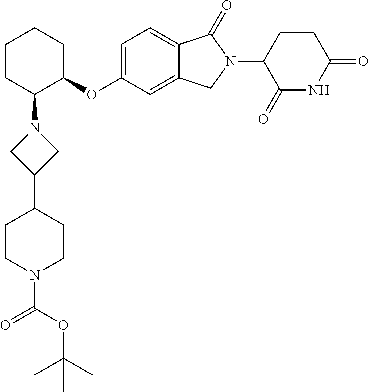 [SMILES: CC(C)(C)OC(=O)N1CCC(C2CN([C@H]3CCCC[C@H]3Oc3ccc4c(c3)CN(C3CCC(=O)NC3=O)C4=O)C2)CC1]

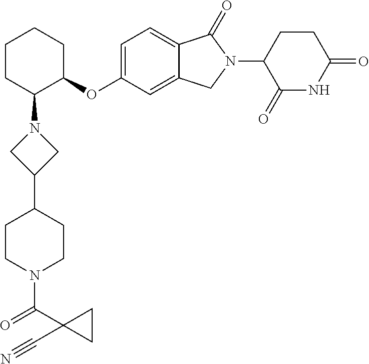 [SMILES: N#CC1(C(=O)N2CCC(C3CN([C@H]4CCCC[C@H]4Oc4ccc5c(c4)CN(C4CCC(=O)NC4=O)C5=O)C3)CC2)CC1]

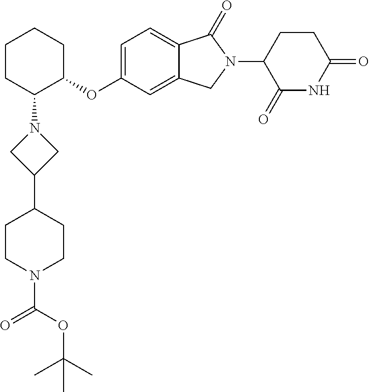 [SMILES: CC(C)(C)OC(=O)N1CCC(C2CN([C@@H]3CCCC[C@@H]3Oc3ccc4c(c3)CN(C3CCC(=O)NC3=O)C4=O)C2)CC1]

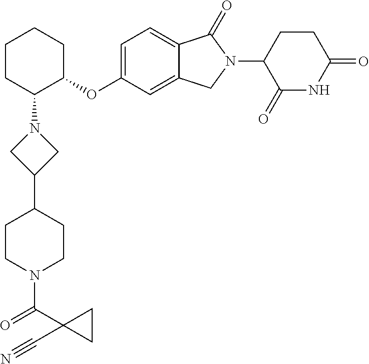 [SMILES: N#CC1(C(=O)N2CCC(C3CN([C@@H]4CCCC[C@@H]4Oc4ccc5c(c4)CN(C4CCC(=O)NC4=O)C5=O)C3)CC2)CC1]

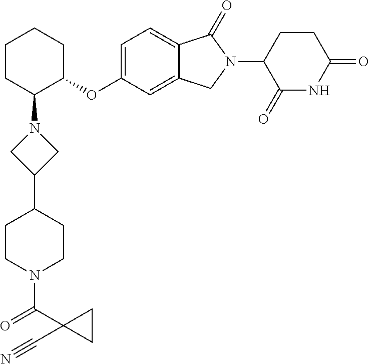 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1]

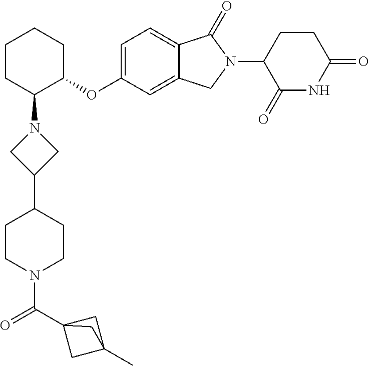 [SMILES: CC12CC(C(=O)N3CCC(C4CN([C@H]5CCCC[C@@H]5Oc5ccc6c(c5)CN(C5CCC(=O)NC5=O)C6=O)C4)CC3)(C1)C2]

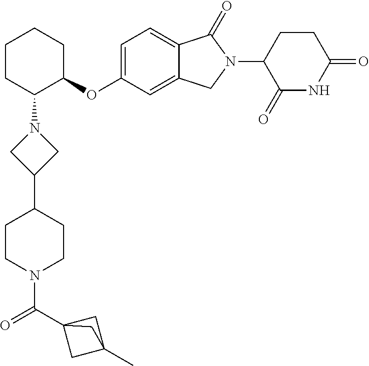 [SMILES: CC12CC(C(=O)N3CCC(C4CN([C@@H]5CCCC[C@H]5Oc5ccc6c(c5)CN(C5CCC(=O)NC5=O)C6=O)C4)CC3)(C1)C2]

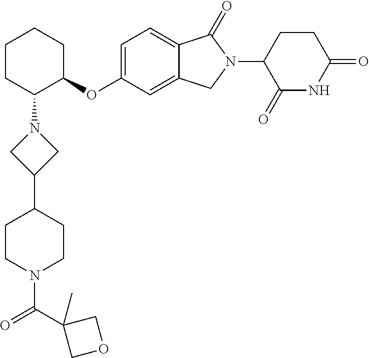 [SMILES: CC1(C(=O)N2CCC(C3CN([C@@H]4CCCC[C@H]4Oc4ccc5c(c4)CN(C4CCC(=O)NC4=O)C5=O)C3)CC2)COC1]

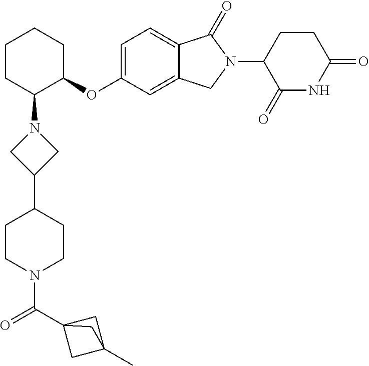 [SMILES: CC12CC(C(=O)N3CCC(C4CN([C@H]5CCCC[C@H]5Oc5ccc6c(c5)CN(C5CCC(=O)NC5=O)C6=O)C4)CC3)(C1)C2]

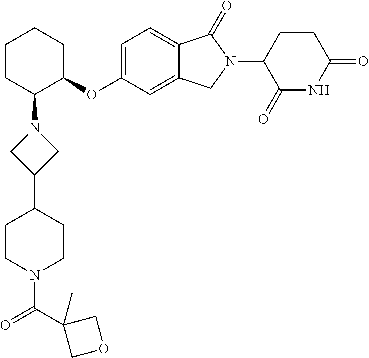 [SMILES: CC1(C(=O)N2CCC(C3CN([C@H]4CCCC[C@H]4Oc4ccc5c(c4)CN(C4CCC(=O)NC4=O)C5=O)C3)CC2)COC1]

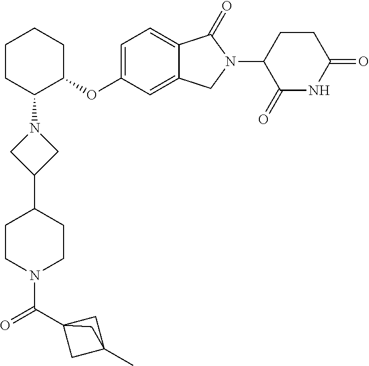 [SMILES: CC12CC(C(=O)N3CCC(C4CN([C@@H]5CCCC[C@@H]5Oc5ccc6c(c5)CN(C5CCC(=O)NC5=O)C6=O)C4)CC3)(C1)C2]

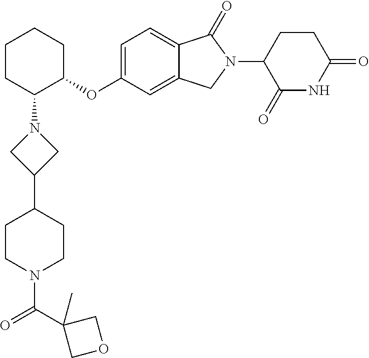 [SMILES: CC1(C(=O)N2CCC(C3CN([C@@H]4CCCC[C@@H]4Oc4ccc5c(c4)CN(C4CCC(=O)NC4=O)C5=O)C3)CC2)COC1]

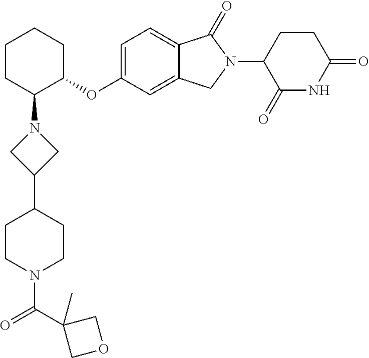 [SMILES: CC1(C(=O)N2CCC(C3CN([C@H]4CCCC[C@@H]4Oc4ccc5c(c4)CN(C4CCC(=O)NC4=O)C5=O)C3)CC2)COC1]

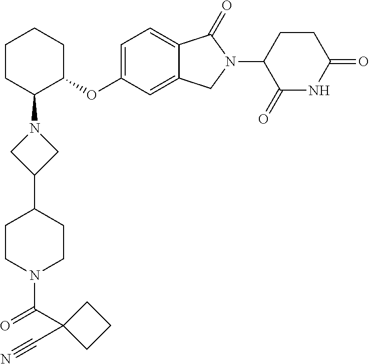 [SMILES: N#CC1(C(=O)N2CCC(C3CN([C@H]4CCCC[C@@H]4Oc4ccc5c(c4)CN(C4CCC(=O)NC4=O)C5=O)C3)CC2)CCC1]

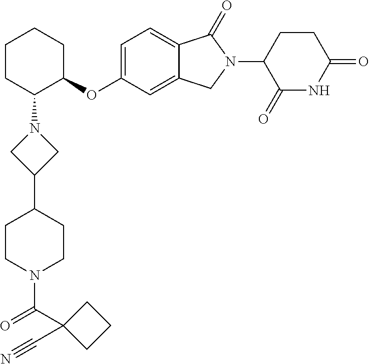 [SMILES: N#CC1(C(=O)N2CCC(C3CN([C@@H]4CCCC[C@H]4Oc4ccc5c(c4)CN(C4CCC(=O)NC4=O)C5=O)C3)CC2)CCC1]

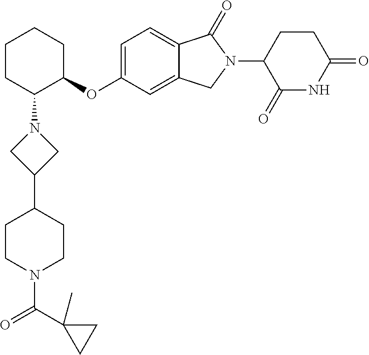 [SMILES: CC1(C(=O)N2CCC(C3CN([C@@H]4CCCC[C@H]4Oc4ccc5c(c4)CN(C4CCC(=O)NC4=O)C5=O)C3)CC2)CC1]

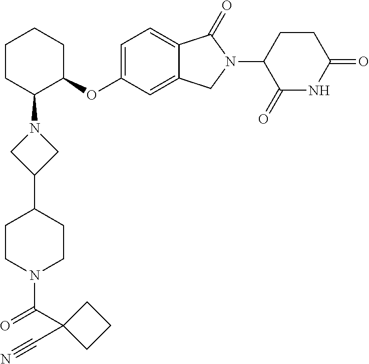 [SMILES: N#CC1(C(=O)N2CCC(C3CN([C@H]4CCCC[C@H]4Oc4ccc5c(c4)CN(C4CCC(=O)NC4=O)C5=O)C3)CC2)CCC1]

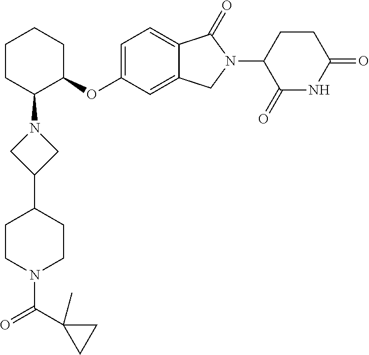 [SMILES: CC1(C(=O)N2CCC(C3CN([C@H]4CCCC[C@H]4Oc4ccc5c(c4)CN(C4CCC(=O)NC4=O)C5=O)C3)CC2)CC1]

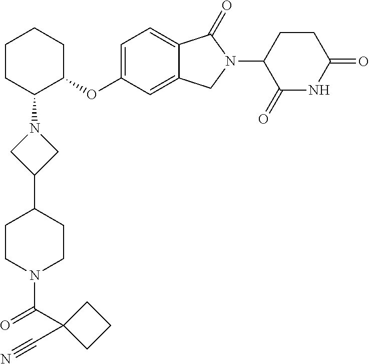 [SMILES: N#CC1(C(=O)N2CCC(C3CN([C@@H]4CCCC[C@@H]4Oc4ccc5c(c4)CN(C4CCC(=O)NC4=O)C5=O)C3)CC2)CCC1]

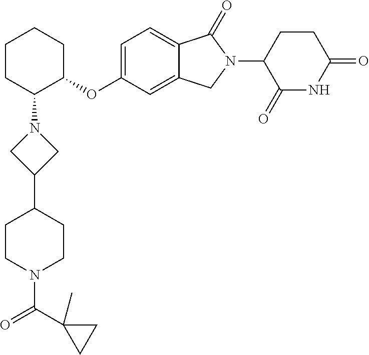 [SMILES: CC1(C(=O)N2CCC(C3CN([C@@H]4CCCC[C@@H]4Oc4ccc5c(c4)CN(C4CCC(=O)NC4=O)C5=O)C3)CC2)CC1]

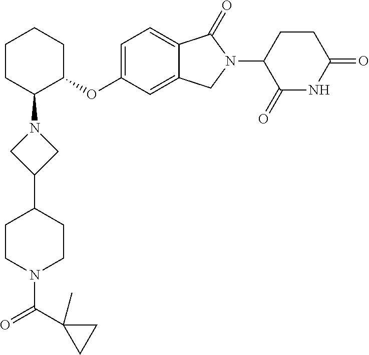 [SMILES: CC1(C(=O)N2CCC(C3CN([C@H]4CCCC[C@@H]4Oc4ccc5c(c4)CN(C4CCC(=O)NC4=O)C5=O)C3)CC2)CC1]

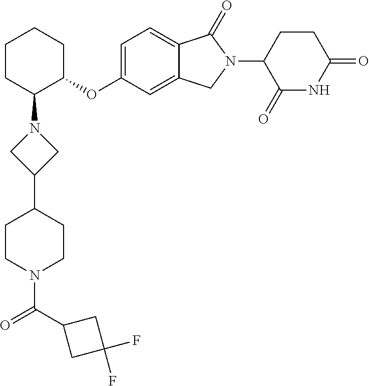 [SMILES: O=C1CCC(N2Cc3cc(O[C@H]4CCCC[C@@H]4N4CC(C5CCN(C(=O)C6CC(F)(F)C6)CC5)C4)ccc3C2=O)C(=O)N1]

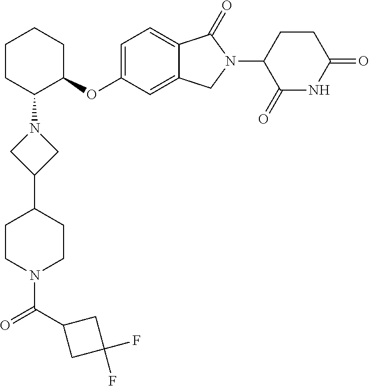 [SMILES: O=C1CCC(N2Cc3cc(O[C@@H]4CCCC[C@H]4N4CC(C5CCN(C(=O)C6CC(F)(F)C6)CC5)C4)ccc3C2=O)C(=O)N1]

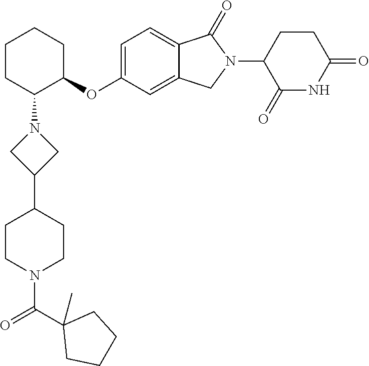 [SMILES: CC1(C(=O)N2CCC(C3CN([C@@H]4CCCC[C@H]4Oc4ccc5c(c4)CN(C4CCC(=O)NC4=O)C5=O)C3)CC2)CCCC1]

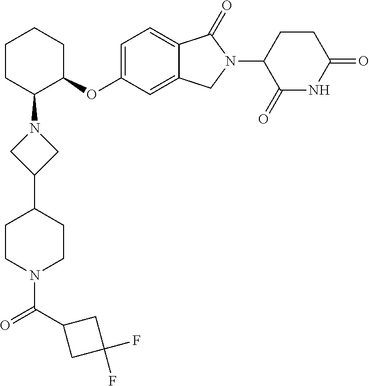 [SMILES: O=C1CCC(N2Cc3cc(O[C@@H]4CCCC[C@@H]4N4CC(C5CCN(C(=O)C6CC(F)(F)C6)CC5)C4)ccc3C2=O)C(=O)N1]

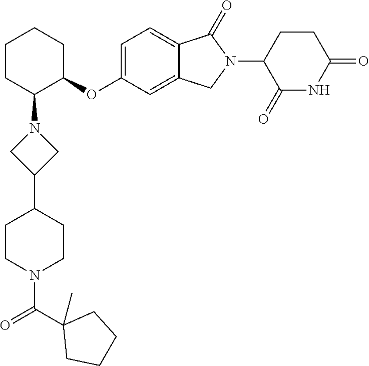 [SMILES: CC1(C(=O)N2CCC(C3CN([C@H]4CCCC[C@H]4Oc4ccc5c(c4)CN(C4CCC(=O)NC4=O)C5=O)C3)CC2)CCCC1]

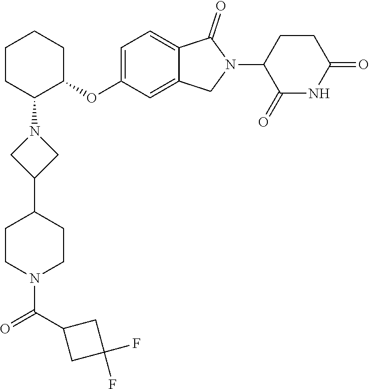 [SMILES: O=C1CCC(N2Cc3cc(O[C@H]4CCCC[C@H]4N4CC(C5CCN(C(=O)C6CC(F)(F)C6)CC5)C4)ccc3C2=O)C(=O)N1]

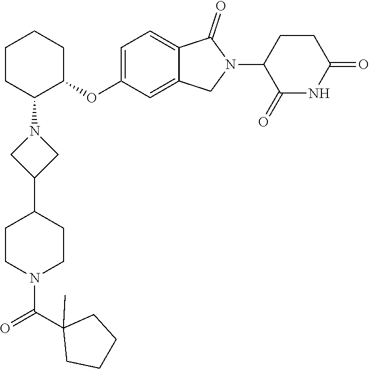 [SMILES: CC1(C(=O)N2CCC(C3CN([C@@H]4CCCC[C@@H]4Oc4ccc5c(c4)CN(C4CCC(=O)NC4=O)C5=O)C3)CC2)CCCC1]

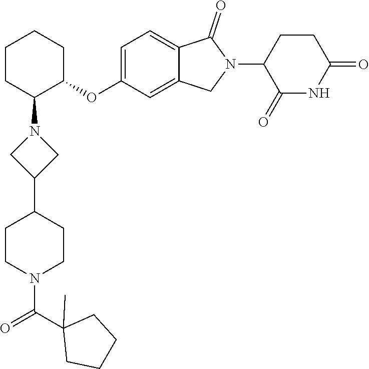 [SMILES: CC1(C(=O)N2CCC(C3CN([C@H]4CCCC[C@@H]4Oc4ccc5c(c4)CN(C4CCC(=O)NC4=O)C5=O)C3)CC2)CCCC1]

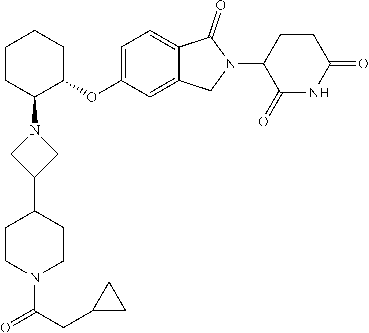 [SMILES: O=C1CCC(N2Cc3cc(O[C@H]4CCCC[C@@H]4N4CC(C5CCN(C(=O)CC6CC6)CC5)C4)ccc3C2=O)C(=O)N1]

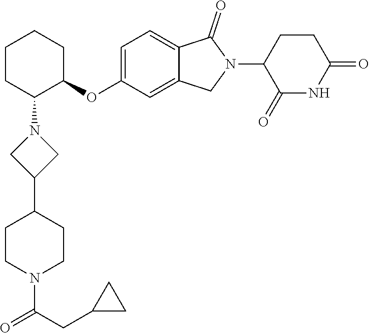 [SMILES: O=C1CCC(N2Cc3cc(O[C@@H]4CCCC[C@H]4N4CC(C5CCN(C(=O)CC6CC6)CC5)C4)ccc3C2=O)C(=O)N1]

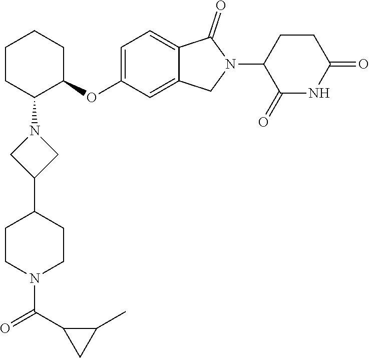 [SMILES: CC1CC1C(=O)N1CCC(C2CN([C@@H]3CCCC[C@H]3Oc3ccc4c(c3)CN(C3CCC(=O)NC3=O)C4=O)C2)CC1]

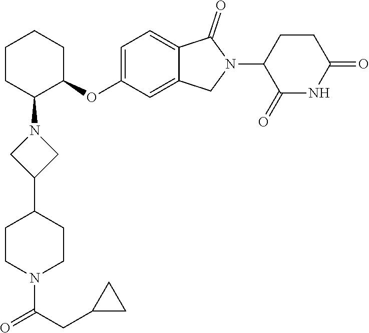 [SMILES: O=C1CCC(N2Cc3cc(O[C@@H]4CCCC[C@@H]4N4CC(C5CCN(C(=O)CC6CC6)CC5)C4)ccc3C2=O)C(=O)N1]

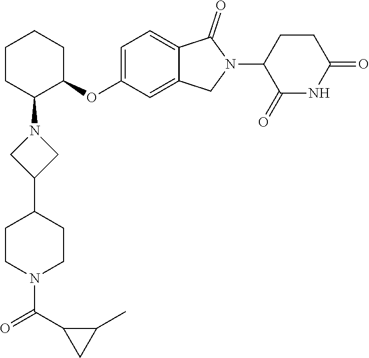 [SMILES: CC1CC1C(=O)N1CCC(C2CN([C@H]3CCCC[C@H]3Oc3ccc4c(c3)CN(C3CCC(=O)NC3=O)C4=O)C2)CC1]

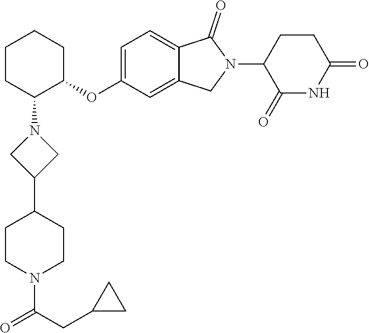 [SMILES: O=C1CCC(N2Cc3cc(O[C@H]4CCCC[C@H]4N4CC(C5CCN(C(=O)CC6CC6)CC5)C4)ccc3C2=O)C(=O)N1]

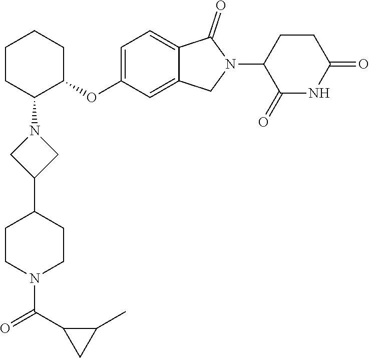 [SMILES: CC1CC1C(=O)N1CCC(C2CN([C@@H]3CCCC[C@@H]3Oc3ccc4c(c3)CN(C3CCC(=O)NC3=O)C4=O)C2)CC1]

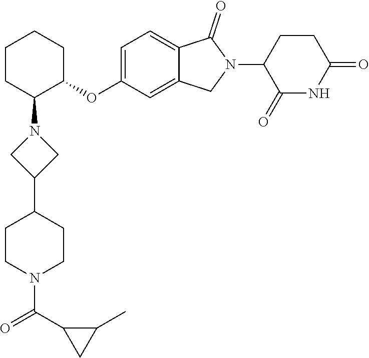 [SMILES: CC1CC1C(=O)N1CCC(C2CN([C@H]3CCCC[C@@H]3Oc3ccc4c(c3)CN(C3CCC(=O)NC3=O)C4=O)C2)CC1]

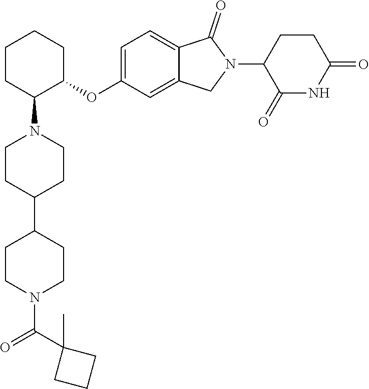 [SMILES: CC1(C(=O)N2CCC(C3CCN([C@H]4CCCC[C@@H]4Oc4ccc5c(c4)CN(C4CCC(=O)NC4=O)C5=O)CC3)CC2)CCC1]

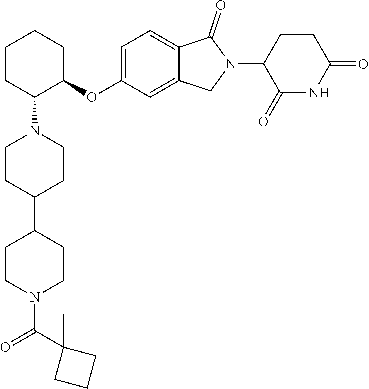 [SMILES: CC1(C(=O)N2CCC(C3CCN([C@@H]4CCCC[C@H]4Oc4ccc5c(c4)CN(C4CCC(=O)NC4=O)C5=O)CC3)CC2)CCC1]

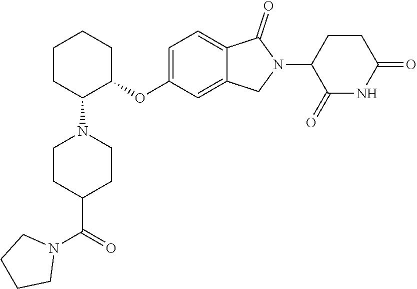 [SMILES: O=C1CCC(N2Cc3cc(O[C@H]4CCCC[C@H]4N4CCC(C(=O)N5CCCC5)CC4)ccc3C2=O)C(=O)N1]

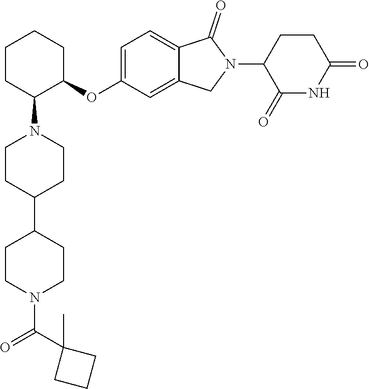 [SMILES: CC1(C(=O)N2CCC(C3CCN([C@H]4CCCC[C@H]4Oc4ccc5c(c4)CN(C4CCC(=O)NC4=O)C5=O)CC3)CC2)CCC1]

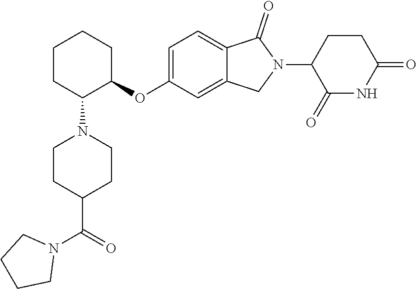 [SMILES: O=C1CCC(N2Cc3cc(O[C@@H]4CCCC[C@H]4N4CCC(C(=O)N5CCCC5)CC4)ccc3C2=O)C(=O)N1]

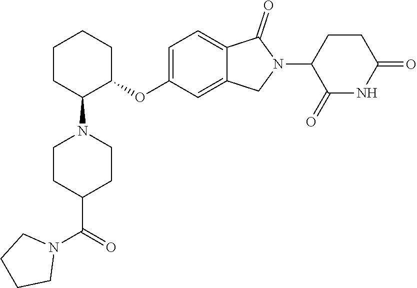 [SMILES: O=C1CCC(N2Cc3cc(O[C@H]4CCCC[C@@H]4N4CCC(C(=O)N5CCCC5)CC4)ccc3C2=O)C(=O)N1]

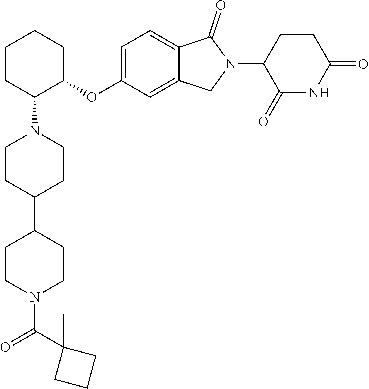 [SMILES: CC1(C(=O)N2CCC(C3CCN([C@@H]4CCCC[C@@H]4Oc4ccc5c(c4)CN(C4CCC(=O)NC4=O)C5=O)CC3)CC2)CCC1]

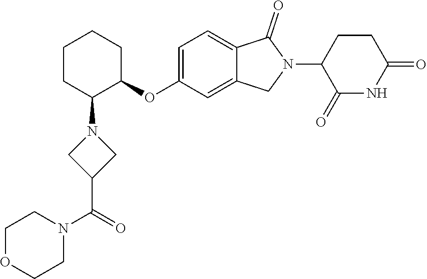 [SMILES: O=C1CCC(N2Cc3cc(O[C@@H]4CCCC[C@@H]4N4CC(C(=O)N5CCOCC5)C4)ccc3C2=O)C(=O)N1]

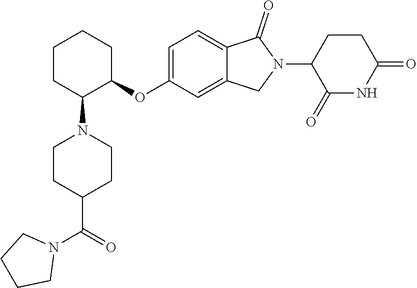 [SMILES: O=C1CCC(N2Cc3cc(O[C@@H]4CCCC[C@@H]4N4CCC(C(=O)N5CCCC5)CC4)ccc3C2=O)C(=O)N1]

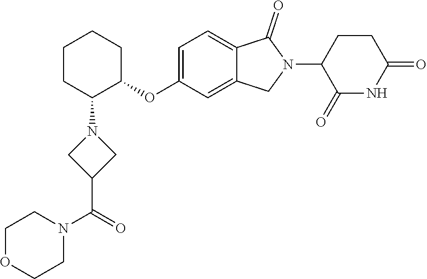 [SMILES: O=C1CCC(N2Cc3cc(O[C@H]4CCCC[C@H]4N4CC(C(=O)N5CCOCC5)C4)ccc3C2=O)C(=O)N1]

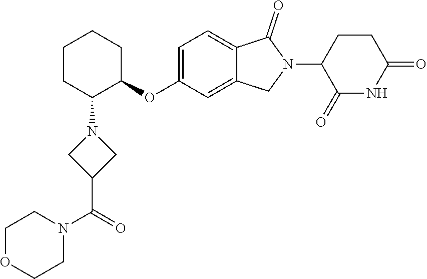 [SMILES: O=C1CCC(N2Cc3cc(O[C@@H]4CCCC[C@H]4N4CC(C(=O)N5CCOCC5)C4)ccc3C2=O)C(=O)N1]

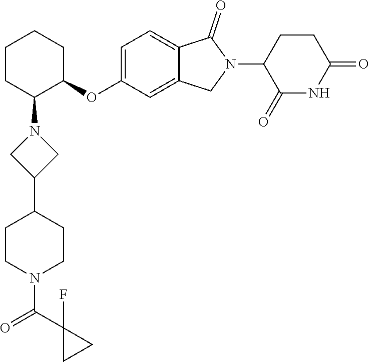 [SMILES: O=C1CCC(N2Cc3cc(O[C@@H]4CCCC[C@@H]4N4CC(C5CCN(C(=O)C6(F)CC6)CC5)C4)ccc3C2=O)C(=O)N1]

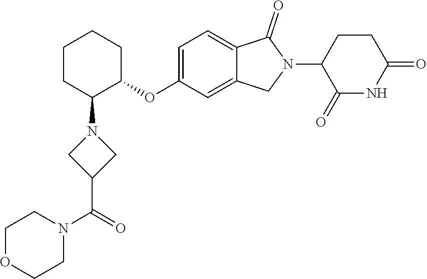 [SMILES: O=C1CCC(N2Cc3cc(O[C@H]4CCCC[C@@H]4N4CC(C(=O)N5CCOCC5)C4)ccc3C2=O)C(=O)N1]

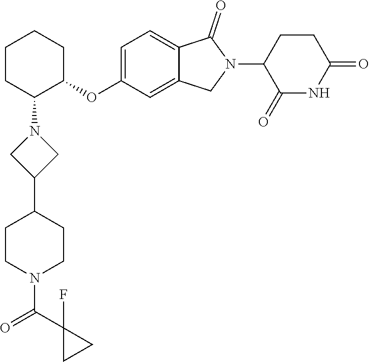 [SMILES: O=C1CCC(N2Cc3cc(O[C@H]4CCCC[C@H]4N4CC(C5CCN(C(=O)C6(F)CC6)CC5)C4)ccc3C2=O)C(=O)N1]

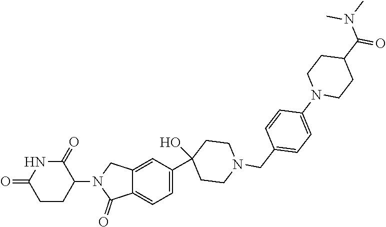 [SMILES: CN(C)C(=O)C1CCN(c2ccc(CN3CCC(O)(c4ccc5c(c4)CN(C4CCC(=O)NC4=O)C5=O)CC3)cc2)CC1]

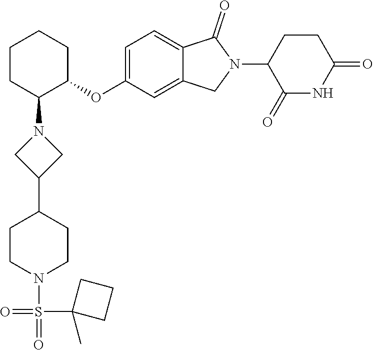 [SMILES: CC1(S(=O)(=O)N2CCC(C3CN([C@H]4CCCC[C@@H]4Oc4ccc5c(c4)CN(C4CCC(=O)NC4=O)C5=O)C3)CC2)CCC1]

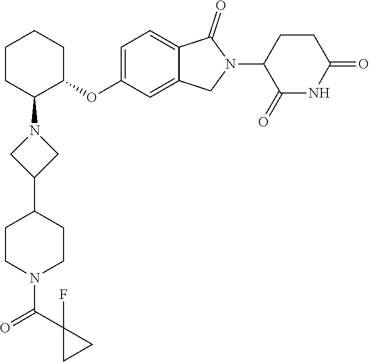 [SMILES: O=C1CCC(N2Cc3cc(O[C@H]4CCCC[C@@H]4N4CC(C5CCN(C(=O)C6(F)CC6)CC5)C4)ccc3C2=O)C(=O)N1]

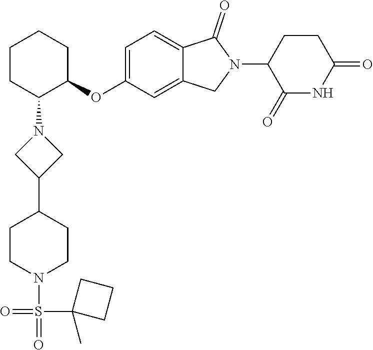 [SMILES: CC1(S(=O)(=O)N2CCC(C3CN([C@@H]4CCCC[C@H]4Oc4ccc5c(c4)CN(C4CCC(=O)NC4=O)C5=O)C3)CC2)CCC1]

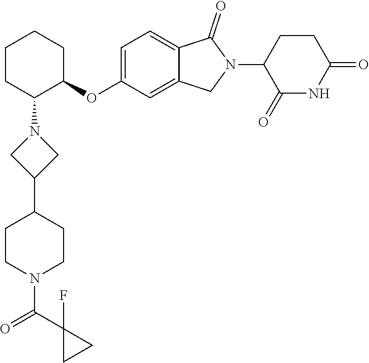 [SMILES: O=C1CCC(N2Cc3cc(O[C@@H]4CCCC[C@H]4N4CC(C5CCN(C(=O)C6(F)CC6)CC5)C4)ccc3C2=O)C(=O)N1]

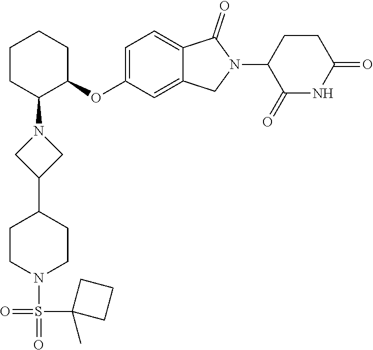 [SMILES: CC1(S(=O)(=O)N2CCC(C3CN([C@H]4CCCC[C@H]4Oc4ccc5c(c4)CN(C4CCC(=O)NC4=O)C5=O)C3)CC2)CCC1]

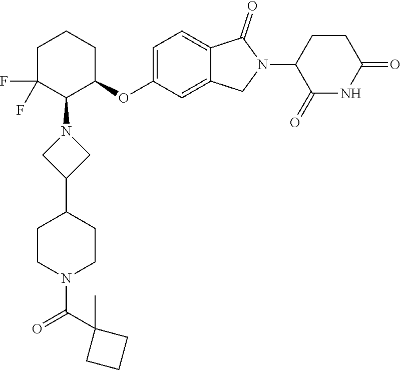 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)CCC1]

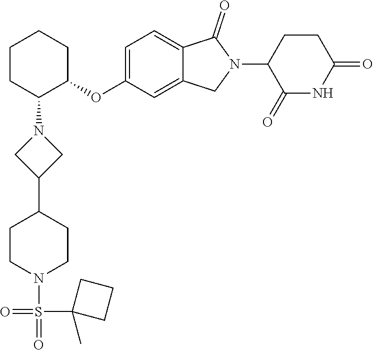 [SMILES: CC1(S(=O)(=O)N2CCC(C3CN([C@@H]4CCCC[C@@H]4Oc4ccc5c(c4)CN(C4CCC(=O)NC4=O)C5=O)C3)CC2)CCC1]

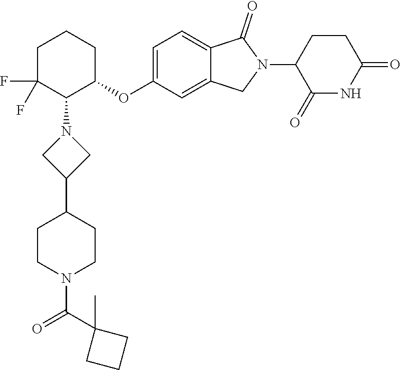 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)CCC1]

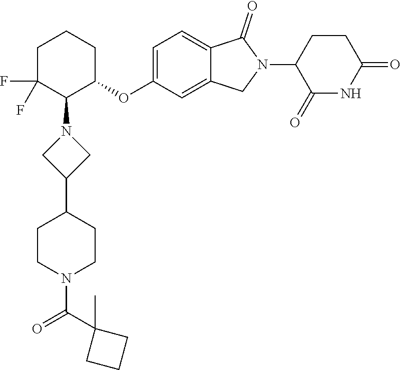 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCC1]

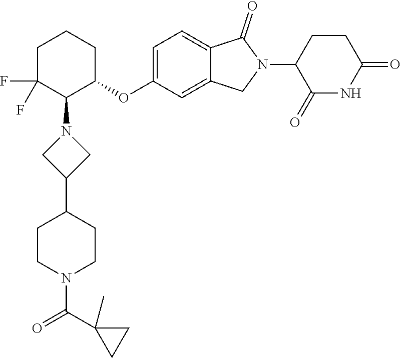 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CC1]

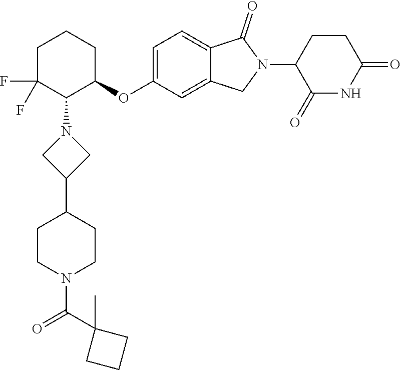 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)CCC1]

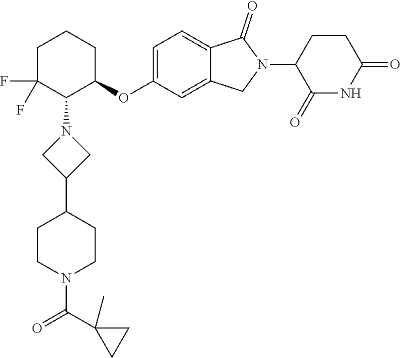 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)CC1]

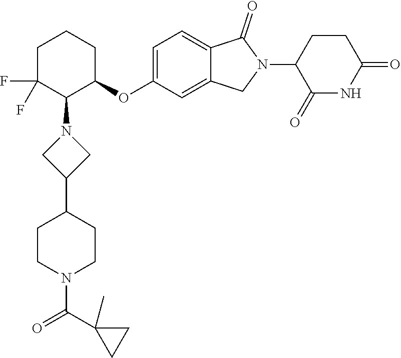 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)CC1]

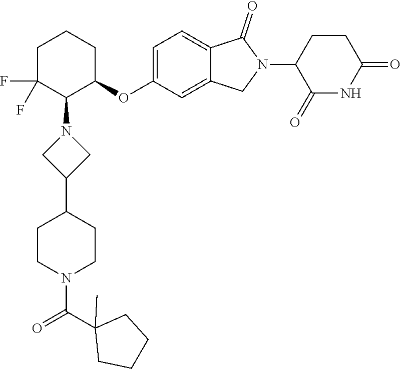 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)CCCC1]

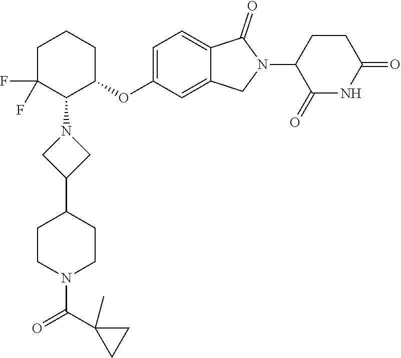 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)CC1]

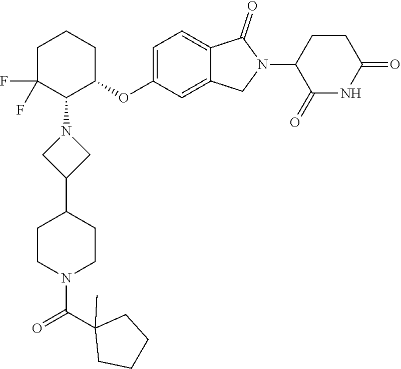 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)CCCC1]

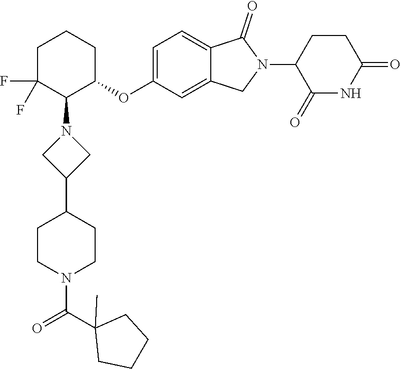 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCCC1]

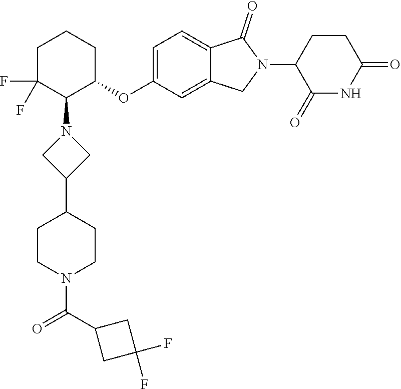 [SMILES: O=C1CCC(N2Cc3cc(O[C@H]4CCCC(F)(F)[C@@H]4N4CC(C5CCN(C(=O)C6CC(F)(F)C6)CC5)C4)ccc3C2=O)C(=O)N1]

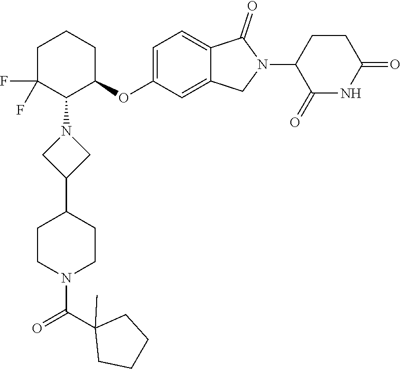 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)CCCC1]

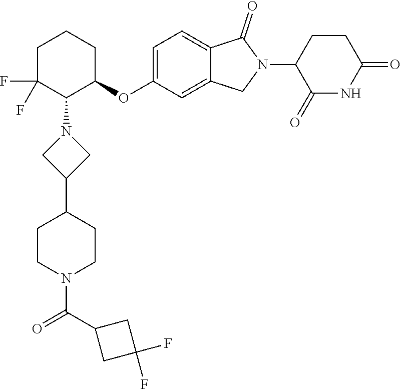 [SMILES: O=C1CCC(N2Cc3cc(O[C@@H]4CCCC(F)(F)[C@H]4N4CC(C5CCN(C(=O)C6CC(F)(F)C6)CC5)C4)ccc3C2=O)C(=O)N1]

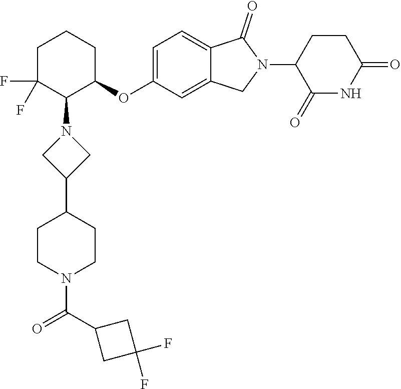 [SMILES: O=C1CCC(N2Cc3cc(O[C@@H]4CCCC(F)(F)[C@@H]4N4CC(C5CCN(C(=O)C6CC(F)(F)C6)CC5)C4)ccc3C2=O)C(=O)N1]

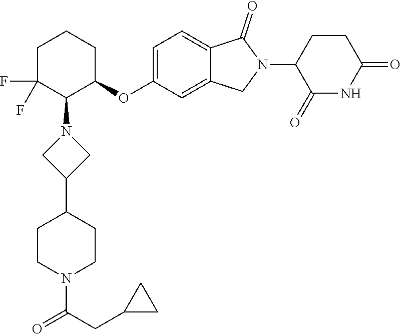 [SMILES: O=C1CCC(N2Cc3cc(O[C@@H]4CCCC(F)(F)[C@@H]4N4CC(C5CCN(C(=O)CC6CC6)CC5)C4)ccc3C2=O)C(=O)N1]

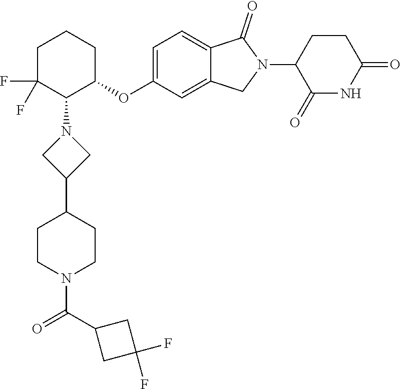 [SMILES: O=C1CCC(N2Cc3cc(O[C@H]4CCCC(F)(F)[C@H]4N4CC(C5CCN(C(=O)C6CC(F)(F)C6)CC5)C4)ccc3C2=O)C(=O)N1]

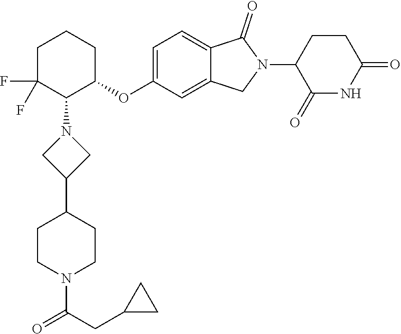 [SMILES: O=C1CCC(N2Cc3cc(O[C@H]4CCCC(F)(F)[C@H]4N4CC(C5CCN(C(=O)CC6CC6)CC5)C4)ccc3C2=O)C(=O)N1]

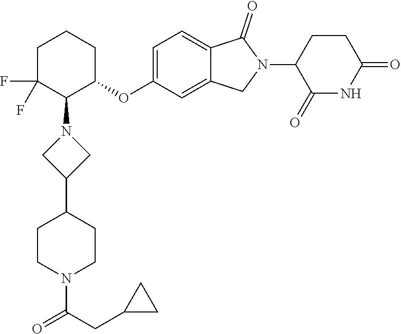 [SMILES: O=C1CCC(N2Cc3cc(O[C@H]4CCCC(F)(F)[C@@H]4N4CC(C5CCN(C(=O)CC6CC6)CC5)C4)ccc3C2=O)C(=O)N1]

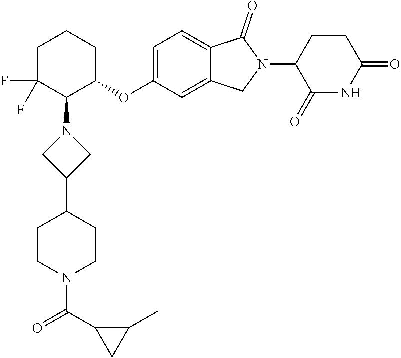 [SMILES: CC1CC1C(=O)N1CCC(C2CN([C@@H]3[C@@H](Oc4ccc5c(c4)CN(C4CCC(=O)NC4=O)C5=O)CCCC3(F)F)C2)CC1]

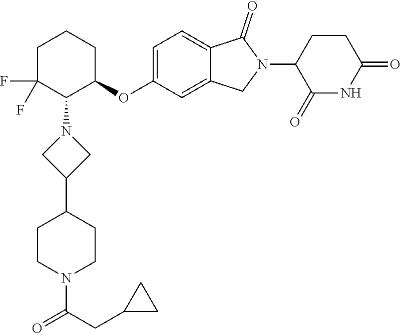 [SMILES: O=C1CCC(N2Cc3cc(O[C@@H]4CCCC(F)(F)[C@H]4N4CC(C5CCN(C(=O)CC6CC6)CC5)C4)ccc3C2=O)C(=O)N1]

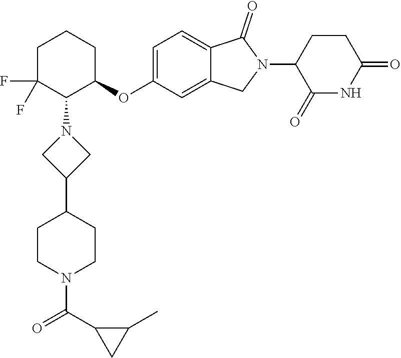 [SMILES: CC1CC1C(=O)N1CCC(C2CN([C@H]3[C@H](Oc4ccc5c(c4)CN(C4CCC(=O)NC4=O)C5=O)CCCC3(F)F)C2)CC1]

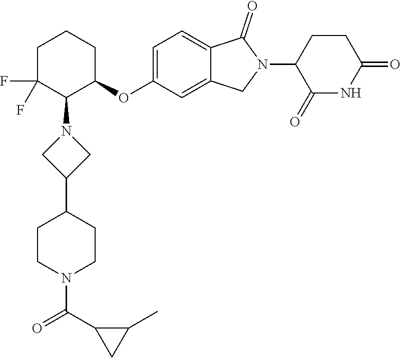 [SMILES: CC1CC1C(=O)N1CCC(C2CN([C@@H]3[C@H](Oc4ccc5c(c4)CN(C4CCC(=O)NC4=O)C5=O)CCCC3(F)F)C2)CC1]

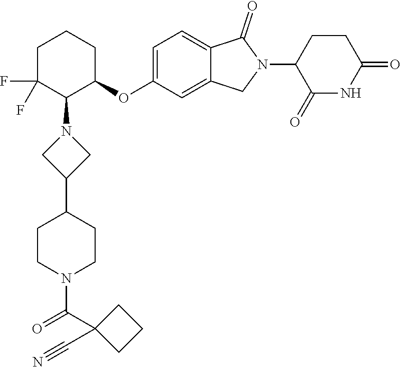 [SMILES: N#CC1(C(=O)N2CCC(C3CN([C@@H]4[C@H](Oc5ccc6c(c5)CN(C5CCC(=O)NC5=O)C6=O)CCCC4(F)F)C3)CC2)CCC1]

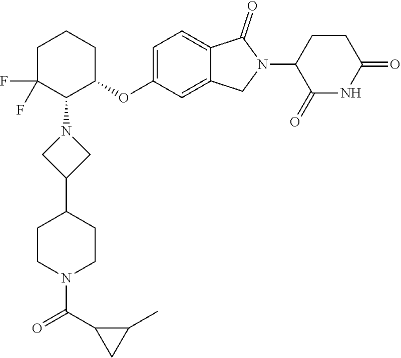 [SMILES: CC1CC1C(=O)N1CCC(C2CN([C@H]3[C@@H](Oc4ccc5c(c4)CN(C4CCC(=O)NC4=O)C5=O)CCCC3(F)F)C2)CC1]

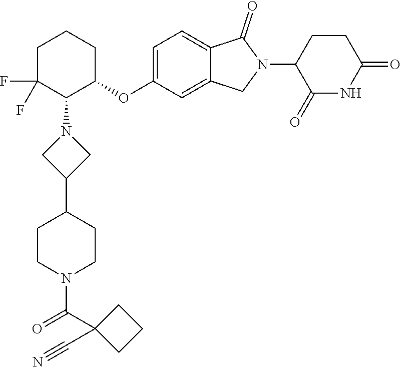 [SMILES: N#CC1(C(=O)N2CCC(C3CN([C@H]4[C@@H](Oc5ccc6c(c5)CN(C5CCC(=O)NC5=O)C6=O)CCCC4(F)F)C3)CC2)CCC1]

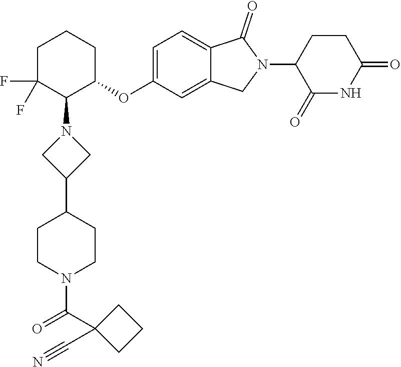 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CCC1]

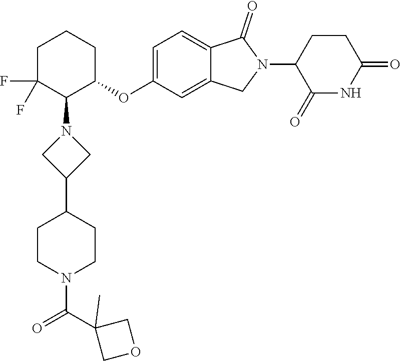 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)COC1]

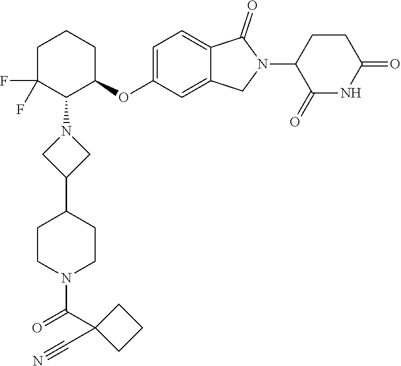 [SMILES: N#CC1(C(=O)N2CCC(C3CN([C@H]4[C@H](Oc5ccc6c(c5)CN(C5CCC(=O)NC5=O)C6=O)CCCC4(F)F)C3)CC2)CCC1]

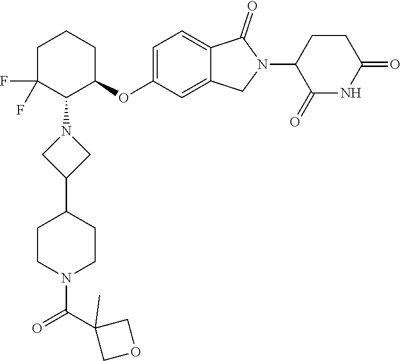 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)COC1]

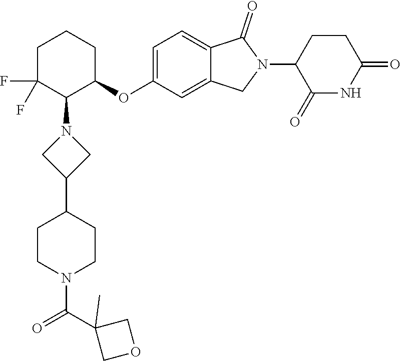 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)COC1]

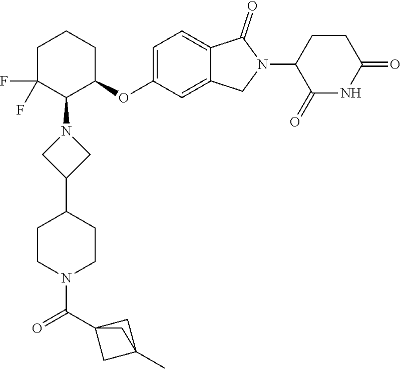 [SMILES: CC12CC(C(=O)N3CCC(C4CN([C@@H]5[C@H](Oc6ccc7c(c6)CN(C6CCC(=O)NC6=O)C7=O)CCCC5(F)F)C4)CC3)(C1)C2]

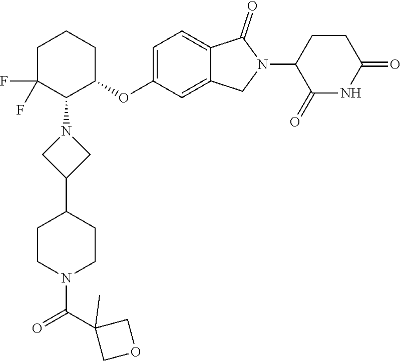 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)COC1]

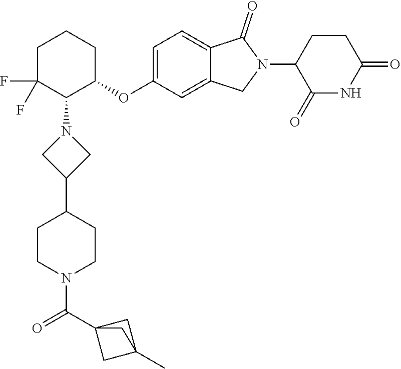 [SMILES: CC12CC(C(=O)N3CCC(C4CN([C@H]5[C@@H](Oc6ccc7c(c6)CN(C6CCC(=O)NC6=O)C7=O)CCCC5(F)F)C4)CC3)(C1)C2]

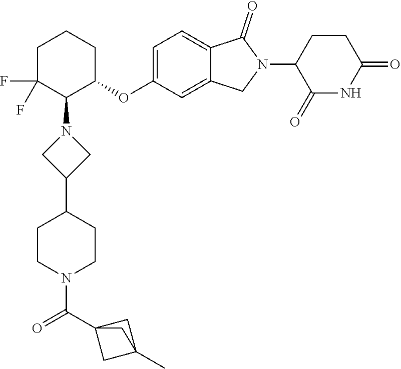 [SMILES: CC12CC(C(=O)N3CCC(C4CN([C@@H]5[C@@H](Oc6ccc7c(c6)CN(C6CCC(=O)NC6=O)C7=O)CCCC5(F)F)C4)CC3)(C1)C2]

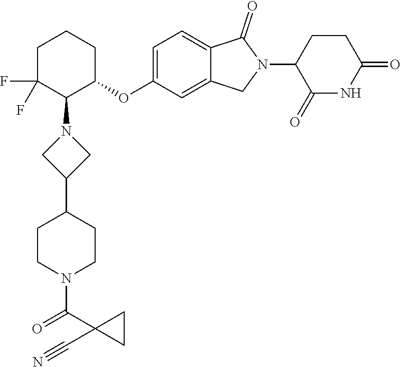 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CC1]

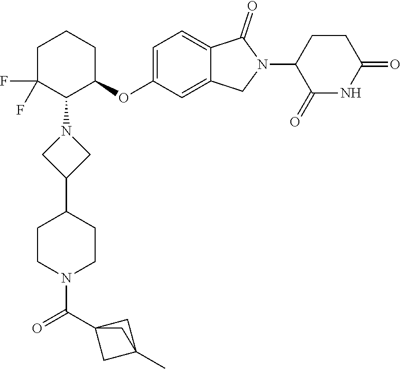 [SMILES: CC12CC(C(=O)N3CCC(C4CN([C@H]5[C@H](Oc6ccc7c(c6)CN(C6CCC(=O)NC6=O)C7=O)CCCC5(F)F)C4)CC3)(C1)C2]

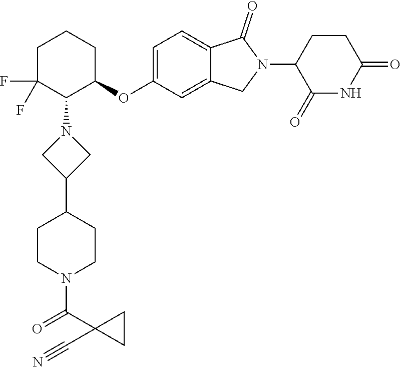 [SMILES: N#CC1(C(=O)N2CCC(C3CN([C@H]4[C@H](Oc5ccc6c(c5)CN(C5CCC(=O)NC5=O)C6=O)CCCC4(F)F)C3)CC2)CC1]

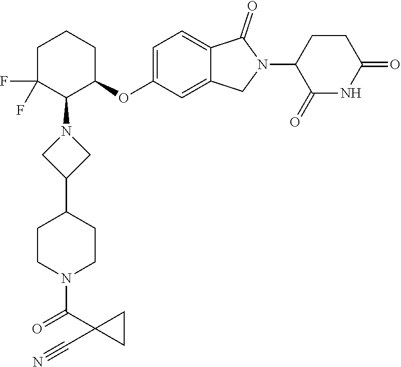 [SMILES: N#CC1(C(=O)N2CCC(C3CN([C@@H]4[C@H](Oc5ccc6c(c5)CN(C5CCC(=O)NC5=O)C6=O)CCCC4(F)F)C3)CC2)CC1]

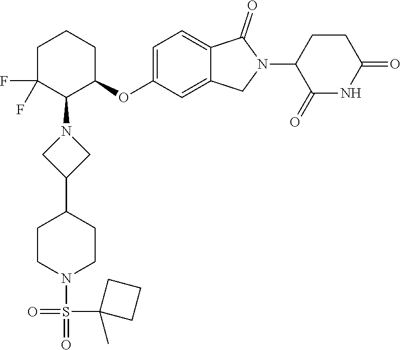 [SMILES: CC1(S(=O)(=O)N2CCC(C3CN([C@@H]4[C@H](Oc5ccc6c(c5)CN(C5CCC(=O)NC5=O)C6=O)CCCC4(F)F)C3)CC2)CCC1]

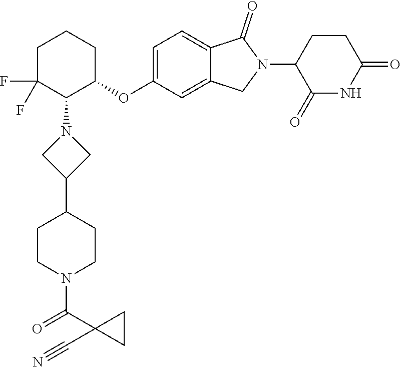 [SMILES: N#CC1(C(=O)N2CCC(C3CN([C@H]4[C@@H](Oc5ccc6c(c5)CN(C5CCC(=O)NC5=O)C6=O)CCCC4(F)F)C3)CC2)CC1]

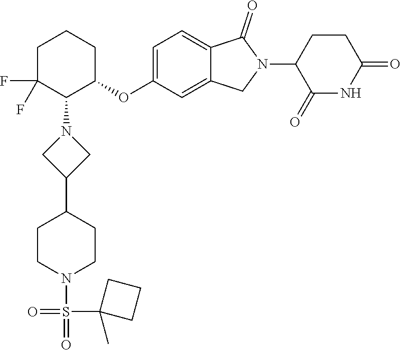 [SMILES: CC1(S(=O)(=O)N2CCC(C3CN([C@H]4[C@@H](Oc5ccc6c(c5)CN(C5CCC(=O)NC5=O)C6=O)CCCC4(F)F)C3)CC2)CCC1]

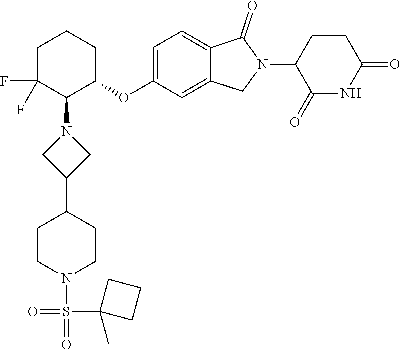 [SMILES: CC1(S(=O)(=O)N2CCC(C3CN([C@@H]4[C@@H](Oc5ccc6c(c5)CN(C5CCC(=O)NC5=O)C6=O)CCCC4(F)F)C3)CC2)CCC1]

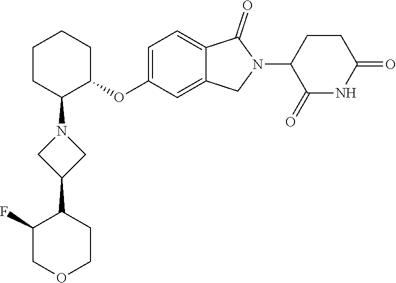

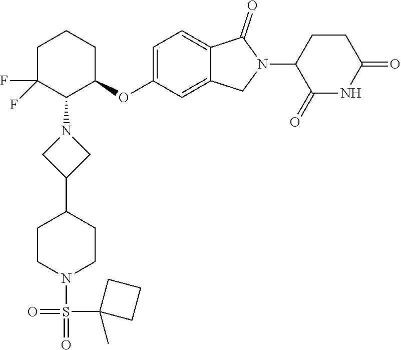 [SMILES: CC1(S(=O)(=O)N2CCC(C3CN([C@H]4[C@H](Oc5ccc6c(c5)CN(C5CCC(=O)NC5=O)C6=O)CCCC4(F)F)C3)CC2)CCC1]

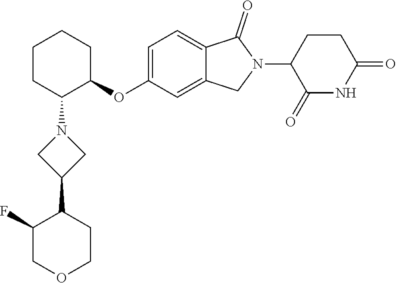

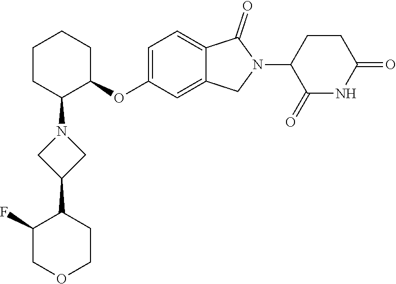

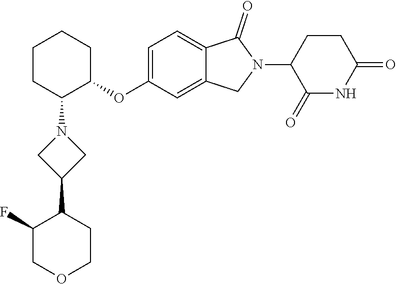

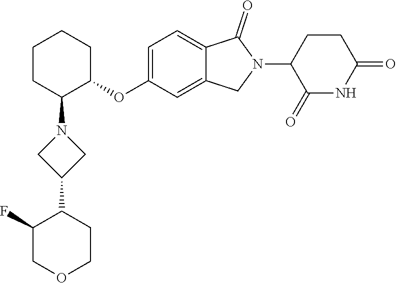

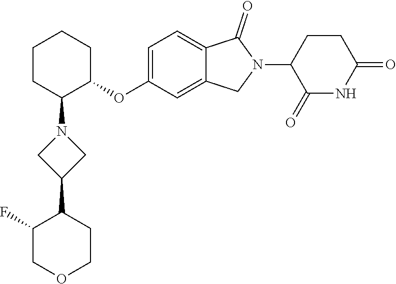

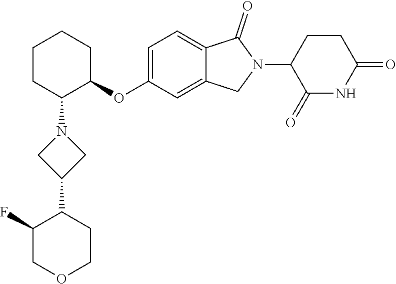

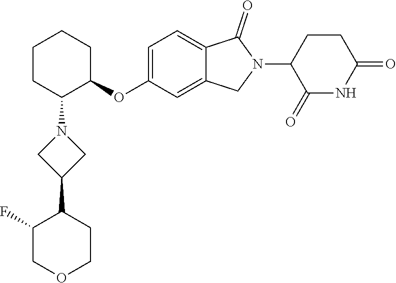

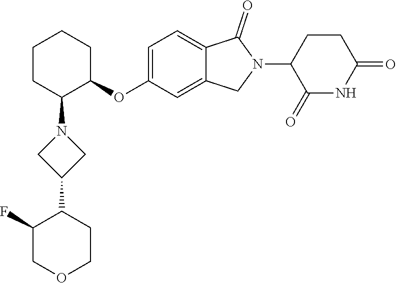

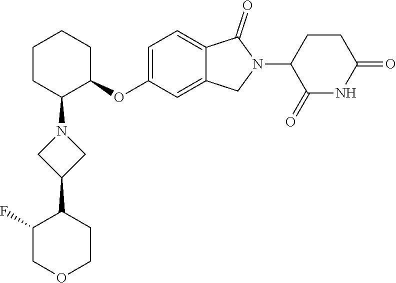

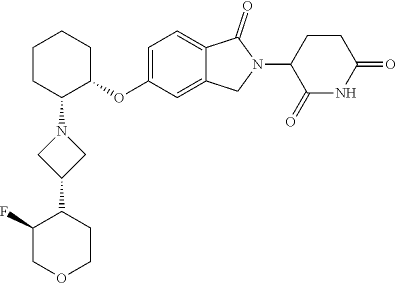

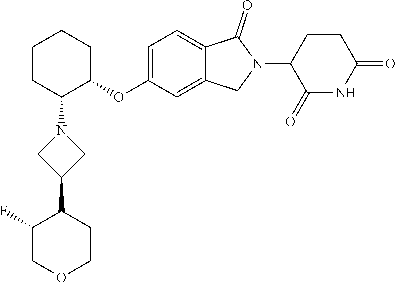

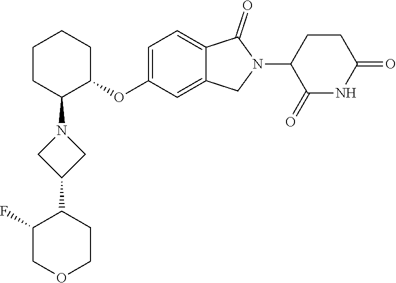

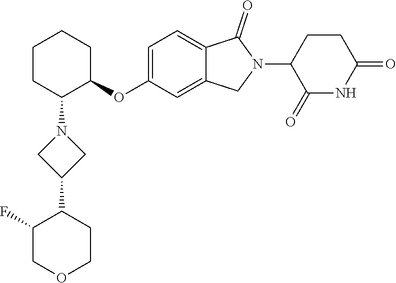

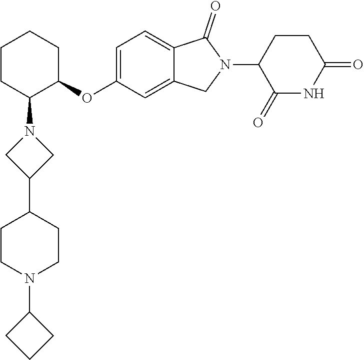 [SMILES: O=C1CCC(N2Cc3cc(O[C@@H]4CCCC[C@@H]4N4CC(C5CCN(C6CCC6)CC5)C4)ccc3C2=O)C(=O)N1]

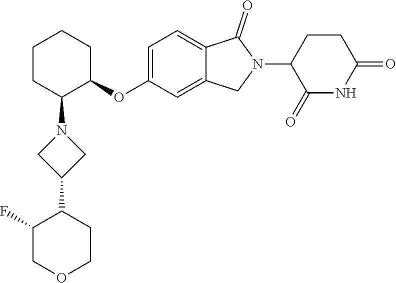

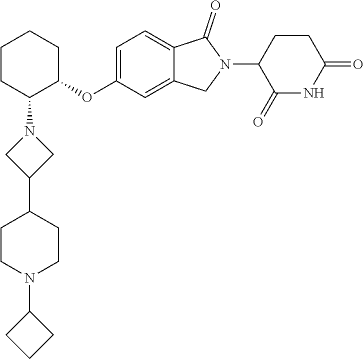 [SMILES: O=C1CCC(N2Cc3cc(O[C@H]4CCCC[C@H]4N4CC(C5CCN(C6CCC6)CC5)C4)ccc3C2=O)C(=O)N1]

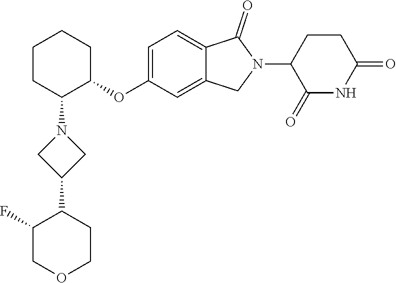

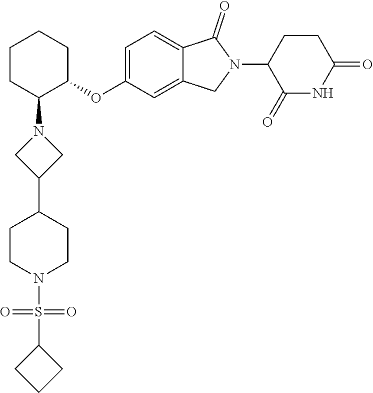 [SMILES: O=C1CCC(N2Cc3cc(O[C@H]4CCCC[C@@H]4N4CC(C5CCN(S(=O)(=O)C6CCC6)CC5)C4)ccc3C2=O)C(=O)N1]

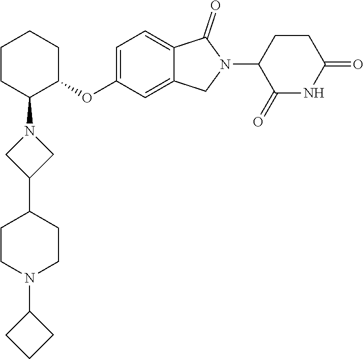 [SMILES: O=C1CCC(N2Cc3cc(O[C@H]4CCCC[C@@H]4N4CC(C5CCN(C6CCC6)CC5)C4)ccc3C2=O)C(=O)N1]

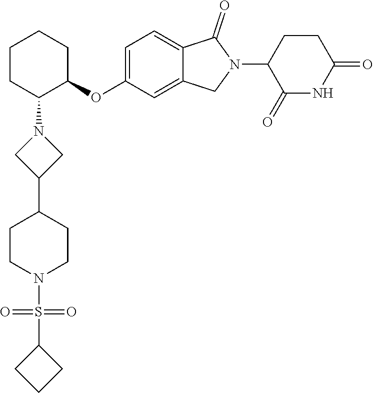 [SMILES: O=C1CCC(N2Cc3cc(O[C@@H]4CCCC[C@H]4N4CC(C5CCN(S(=O)(=O)C6CCC6)CC5)C4)ccc3C2=O)C(=O)N1]

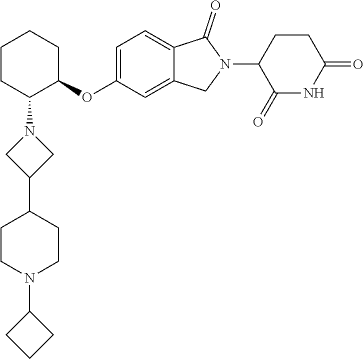 [SMILES: O=C1CCC(N2Cc3cc(O[C@@H]4CCCC[C@H]4N4CC(C5CCN(C6CCC6)CC5)C4)ccc3C2=O)C(=O)N1]

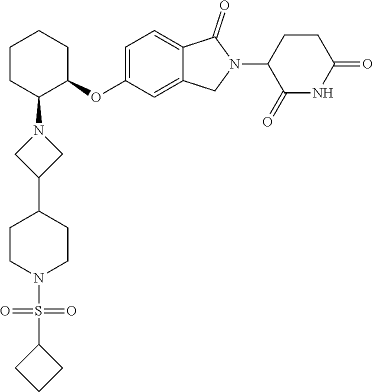 [SMILES: O=C1CCC(N2Cc3cc(O[C@@H]4CCCC[C@@H]4N4CC(C5CCN(S(=O)(=O)C6CCC6)CC5)C4)ccc3C2=O)C(=O)N1]

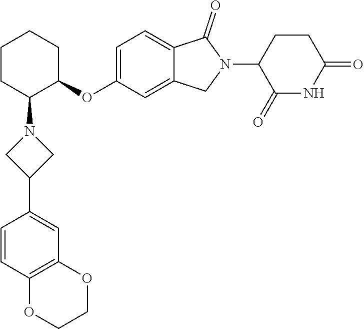 [SMILES: O=C1CCC(N2Cc3cc(O[C@@H]4CCCC[C@@H]4N4CC(c5ccc6c(c5)OCCO6)C4)ccc3C2=O)C(=O)N1]

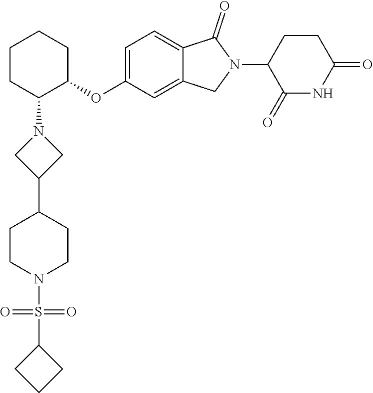 [SMILES: O=C1CCC(N2Cc3cc(O[C@H]4CCCC[C@H]4N4CC(C5CCN(S(=O)(=O)C6CCC6)CC5)C4)ccc3C2=O)C(=O)N1]

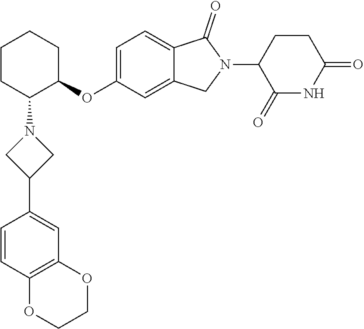 [SMILES: O=C1CCC(N2Cc3cc(O[C@@H]4CCCC[C@H]4N4CC(c5ccc6c(c5)OCCO6)C4)ccc3C2=O)C(=O)N1]

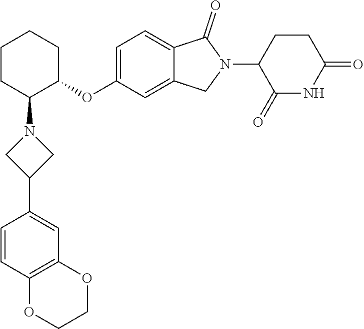 [SMILES: O=C1CCC(N2Cc3cc(O[C@H]4CCCC[C@@H]4N4CC(c5ccc6c(c5)OCCO6)C4)ccc3C2=O)C(=O)N1]

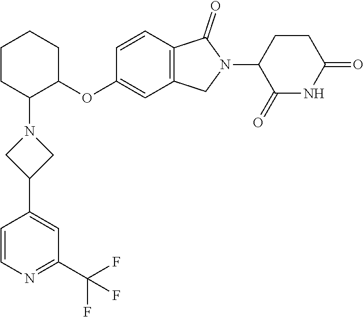 [SMILES: O=C1CCC(N2Cc3cc(OC4CCCCC4N4CC(c5ccnc(C(F)(F)F)c5)C4)ccc3C2=O)C(=O)N1]

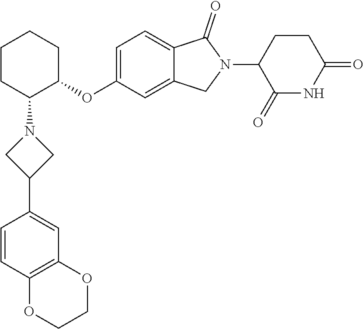 [SMILES: O=C1CCC(N2Cc3cc(O[C@H]4CCCC[C@H]4N4CC(c5ccc6c(c5)OCCO6)C4)ccc3C2=O)C(=O)N1]

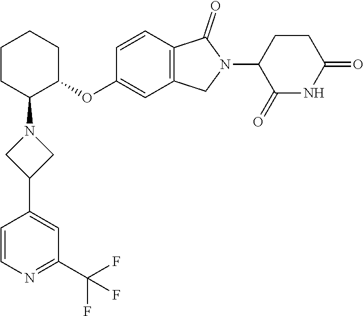 [SMILES: O=C1CCC(N2Cc3cc(O[C@H]4CCCC[C@@H]4N4CC(c5ccnc(C(F)(F)F)c5)C4)ccc3C2=O)C(=O)N1]

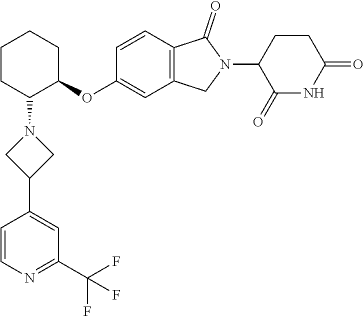

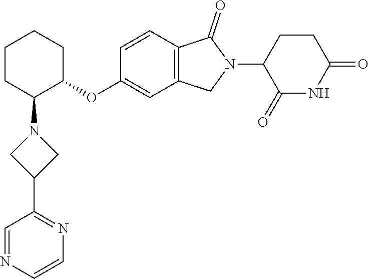 [SMILES: O=C1CCC(N2Cc3cc(O[C@H]4CCCC[C@@H]4N4CC(c5cnccn5)C4)ccc3C2=O)C(=O)N1]

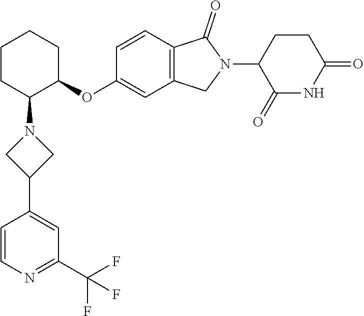 [SMILES: O=C1CCC(N2Cc3cc(O[C@@H]4CCCC[C@@H]4N4CC(c5ccnc(C(F)(F)F)c5)C4)ccc3C2=O)C(=O)N1]

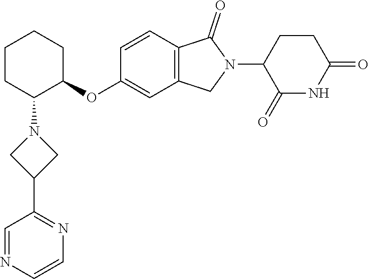 [SMILES: O=C1CCC(N2Cc3cc(O[C@@H]4CCCC[C@H]4N4CC(c5cnccn5)C4)ccc3C2=O)C(=O)N1]

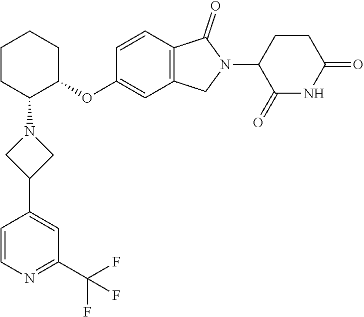 [SMILES: O=C1CCC(N2Cc3cc(O[C@H]4CCCC[C@H]4N4CC(c5ccnc(C(F)(F)F)c5)C4)ccc3C2=O)C(=O)N1]

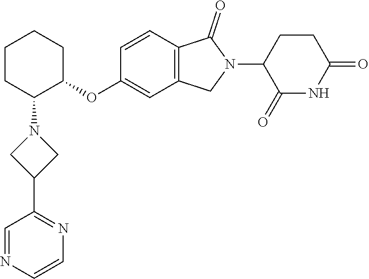 [SMILES: O=C1CCC(N2Cc3cc(O[C@H]4CCCC[C@H]4N4CC(c5cnccn5)C4)ccc3C2=O)C(=O)N1]

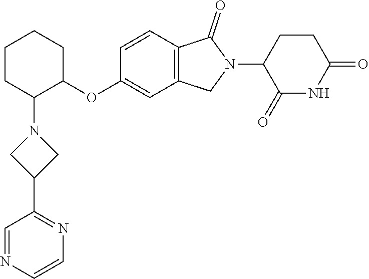 [SMILES: O=C1CCC(N2Cc3cc(OC4CCCCC4N4CC(c5cnccn5)C4)ccc3C2=O)C(=O)N1]

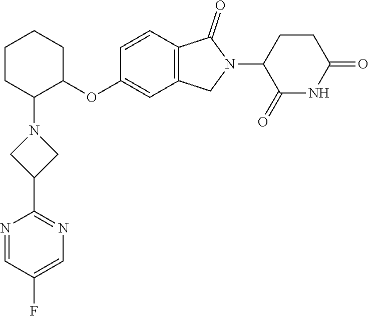 [SMILES: O=C1CCC(N2Cc3cc(OC4CCCCC4N4CC(c5ncc(F)cn5)C4)ccc3C2=O)C(=O)N1]

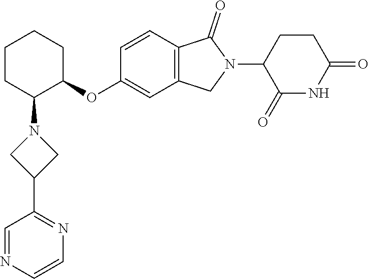 [SMILES: O=C1CCC(N2Cc3cc(O[C@@H]4CCCC[C@@H]4N4CC(c5cnccn5)C4)ccc3C2=O)C(=O)N1]

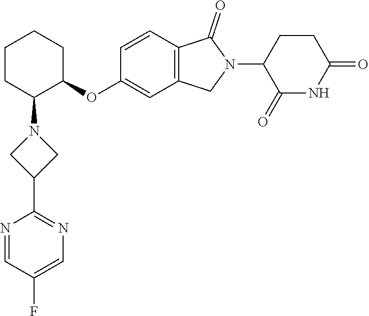 [SMILES: O=C1CCC(N2Cc3cc(O[C@@H]4CCCC[C@@H]4N4CC(c5ncc(F)cn5)C4)ccc3C2=O)C(=O)N1]

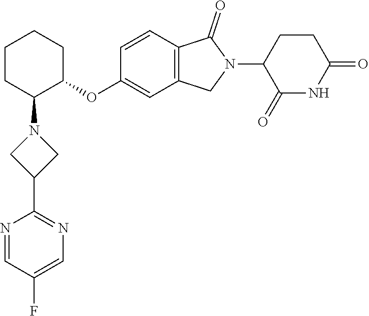 [SMILES: O=C1CCC(N2Cc3cc(O[C@H]4CCCC[C@@H]4N4CC(c5ncc(F)cn5)C4)ccc3C2=O)C(=O)N1]

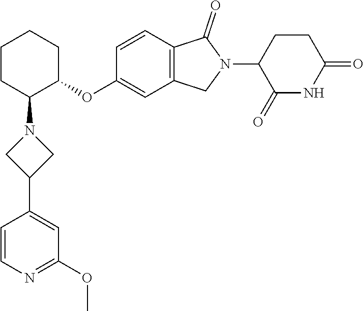 [SMILES: COc1cc(C2CN([C@H]3CCCC[C@@H]3Oc3ccc4c(c3)CN(C3CCC(=O)NC3=O)C4=O)C2)ccn1]

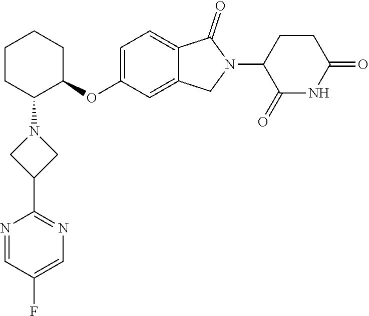 [SMILES: O=C1CCC(N2Cc3cc(O[C@@H]4CCCC[C@H]4N4CC(c5ncc(F)cn5)C4)ccc3C2=O)C(=O)N1]

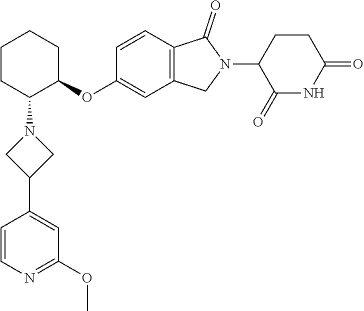 [SMILES: COc1cc(C2CN([C@@H]3CCCC[C@H]3Oc3ccc4c(c3)CN(C3CCC(=O)NC3=O)C4=O)C2)ccn1]

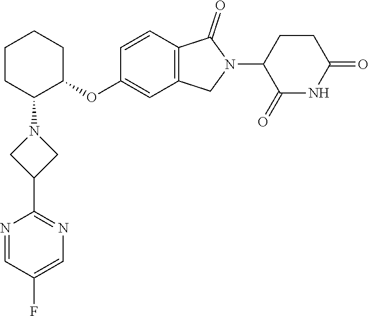 [SMILES: O=C1CCC(N2Cc3cc(O[C@H]4CCCC[C@H]4N4CC(c5ncc(F)cn5)C4)ccc3C2=O)C(=O)N1]

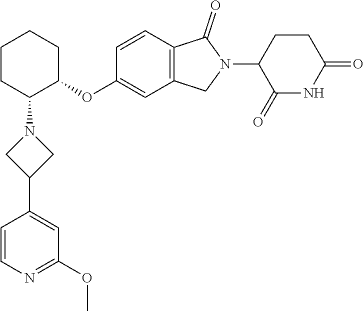 [SMILES: COc1cc(C2CN([C@@H]3CCCC[C@@H]3Oc3ccc4c(c3)CN(C3CCC(=O)NC3=O)C4=O)C2)ccn1]

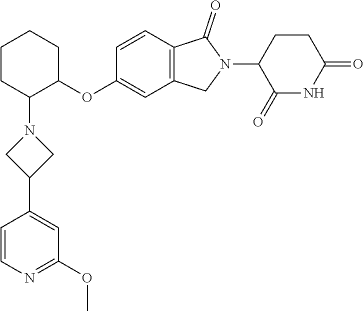 [SMILES: COc1cc(C2CN(C3CCCCC3Oc3ccc4c(c3)CN(C3CCC(=O)NC3=O)C4=O)C2)ccn1]

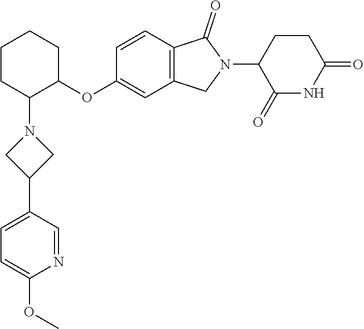 [SMILES: COc1ccc(C2CN(C3CCCCC3Oc3ccc4c(c3)CN(C3CCC(=O)NC3=O)C4=O)C2)cn1]

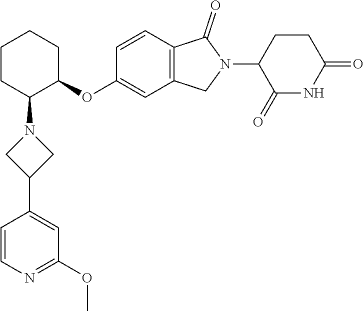 [SMILES: COc1cc(C2CN([C@H]3CCCC[C@H]3Oc3ccc4c(c3)CN(C3CCC(=O)NC3=O)C4=O)C2)ccn1]

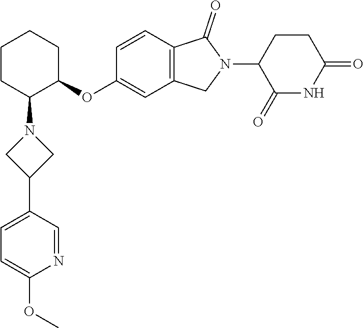 [SMILES: COc1ccc(C2CN([C@H]3CCCC[C@H]3Oc3ccc4c(c3)CN(C3CCC(=O)NC3=O)C4=O)C2)cn1]

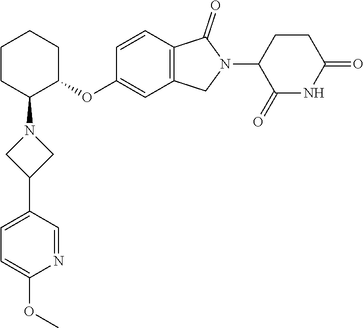 [SMILES: COc1ccc(C2CN([C@H]3CCCC[C@@H]3Oc3ccc4c(c3)CN(C3CCC(=O)NC3=O)C4=O)C2)cn1]

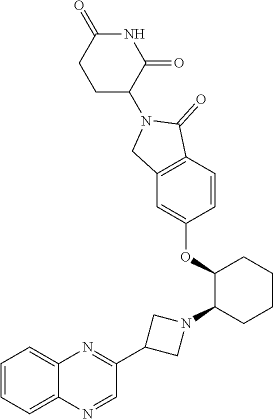 [SMILES: O=C1CCC(N2Cc3cc(O[C@H]4CCCC[C@H]4N4CC(c5cnc6ccccc6n5)C4)ccc3C2=O)C(=O)N1]

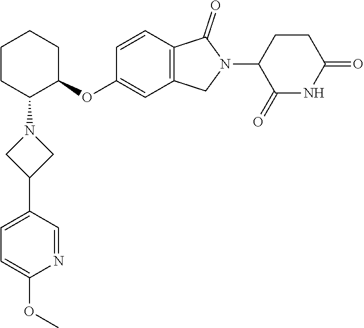 [SMILES: COc1ccc(C2CN([C@@H]3CCCC[C@H]3Oc3ccc4c(c3)CN(C3CCC(=O)NC3=O)C4=O)C2)cn1]

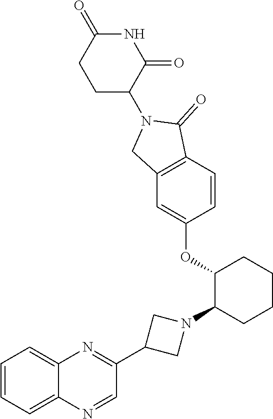 [SMILES: O=C1CCC(N2Cc3cc(O[C@@H]4CCCC[C@H]4N4CC(c5cnc6ccccc6n5)C4)ccc3C2=O)C(=O)N1]

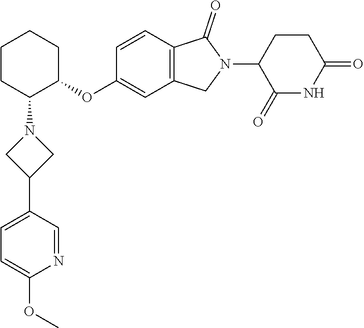 [SMILES: COc1ccc(C2CN([C@@H]3CCCC[C@@H]3Oc3ccc4c(c3)CN(C3CCC(=O)NC3=O)C4=O)C2)cn1]

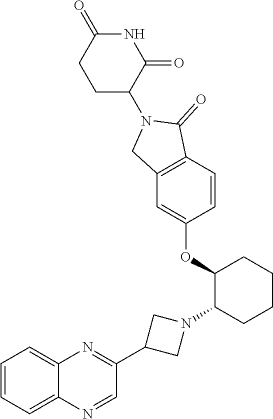 [SMILES: O=C1CCC(N2Cc3cc(O[C@H]4CCCC[C@@H]4N4CC(c5cnc6ccccc6n5)C4)ccc3C2=O)C(=O)N1]

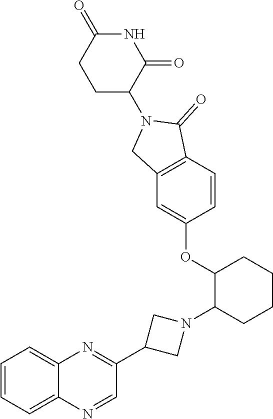 [SMILES: O=C1CCC(N2Cc3cc(OC4CCCCC4N4CC(c5cnc6ccccc6n5)C4)ccc3C2=O)C(=O)N1]

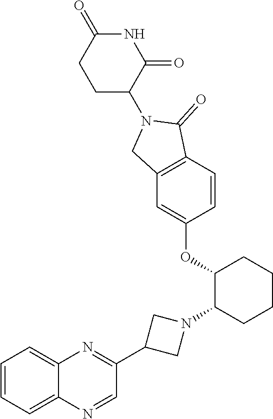 [SMILES: O=C1CCC(N2Cc3cc(O[C@@H]4CCCC[C@@H]4N4CC(c5cnc6ccccc6n5)C4)ccc3C2=O)C(=O)N1]

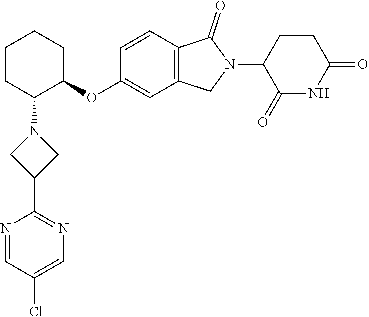 [SMILES: O=C1CCC(N2Cc3cc(O[C@@H]4CCCC[C@H]4N4CC(c5ncc(Cl)cn5)C4)ccc3C2=O)C(=O)N1]

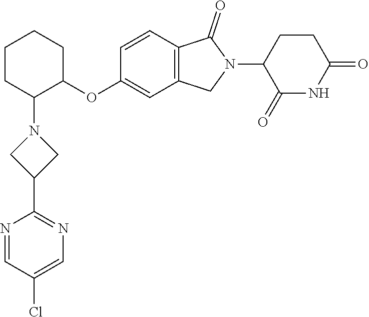 [SMILES: O=C1CCC(N2Cc3cc(OC4CCCCC4N4CC(c5ncc(Cl)cn5)C4)ccc3C2=O)C(=O)N1]

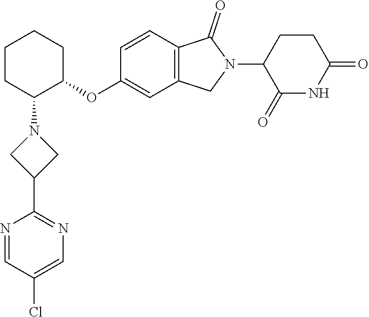 [SMILES: O=C1CCC(N2Cc3cc(O[C@H]4CCCC[C@H]4N4CC(c5ncc(Cl)cn5)C4)ccc3C2=O)C(=O)N1]

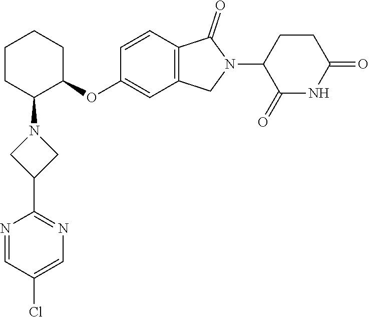 [SMILES: O=C1CCC(N2Cc3cc(O[C@@H]4CCCC[C@@H]4N4CC(c5ncc(Cl)cn5)C4)ccc3C2=O)C(=O)N1]

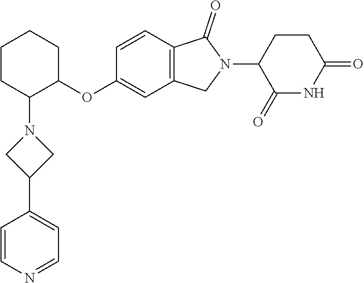 [SMILES: O=C1CCC(N2Cc3cc(OC4CCCCC4N4CC(c5ccncc5)C4)ccc3C2=O)C(=O)N1]

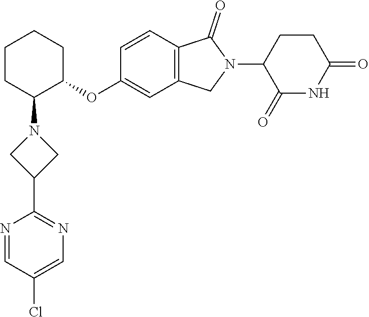 [SMILES: O=C1CCC(N2Cc3cc(O[C@H]4CCCC[C@@H]4N4CC(c5ncc(Cl)cn5)C4)ccc3C2=O)C(=O)N1]

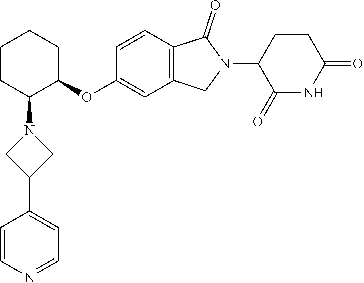 [SMILES: O=C1CCC(N2Cc3cc(O[C@@H]4CCCC[C@@H]4N4CC(c5ccncc5)C4)ccc3C2=O)C(=O)N1]

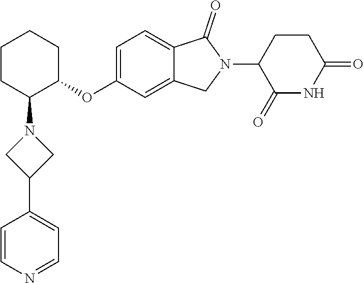 [SMILES: O=C1CCC(N2Cc3cc(O[C@H]4CCCC[C@@H]4N4CC(c5ccncc5)C4)ccc3C2=O)C(=O)N1]

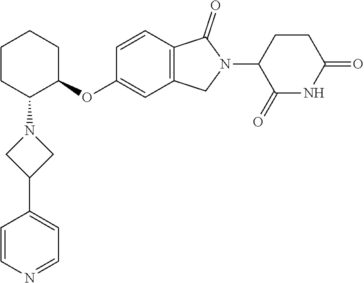 [SMILES: O=C1CCC(N2Cc3cc(O[C@@H]4CCCC[C@H]4N4CC(c5ccncc5)C4)ccc3C2=O)C(=O)N1]

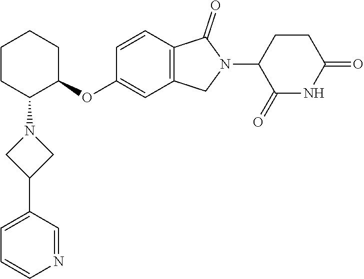 [SMILES: O=C1CCC(N2Cc3cc(O[C@@H]4CCCC[C@H]4N4CC(c5cccnc5)C4)ccc3C2=O)C(=O)N1]

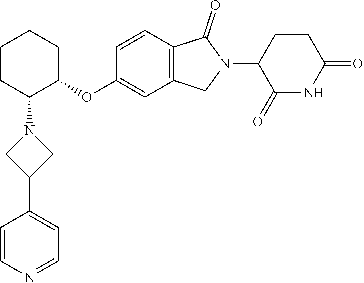 [SMILES: O=C1CCC(N2Cc3cc(O[C@H]4CCCC[C@H]4N4CC(c5ccncc5)C4)ccc3C2=O)C(=O)N1]

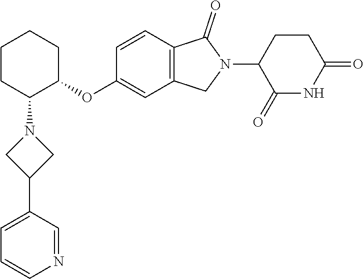 [SMILES: O=C1CCC(N2Cc3cc(O[C@H]4CCCC[C@H]4N4CC(c5cccnc5)C4)ccc3C2=O)C(=O)N1]

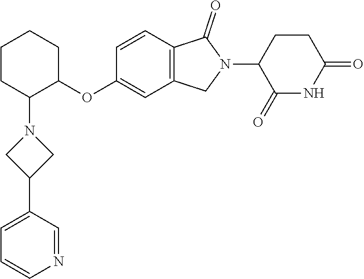 [SMILES: O=C1CCC(N2Cc3cc(OC4CCCCC4N4CC(c5cccnc5)C4)ccc3C2=O)C(=O)N1]

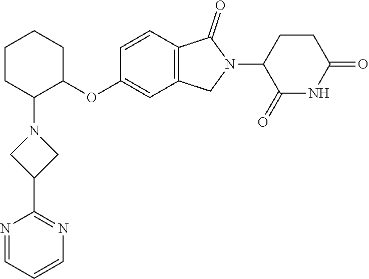 [SMILES: O=C1CCC(N2Cc3cc(OC4CCCCC4N4CC(c5ncccn5)C4)ccc3C2=O)C(=O)N1]

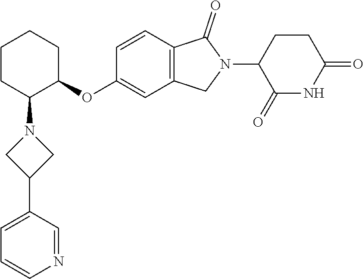 [SMILES: O=C1CCC(N2Cc3cc(O[C@@H]4CCCC[C@@H]4N4CC(c5cccnc5)C4)ccc3C2=O)C(=O)N1]

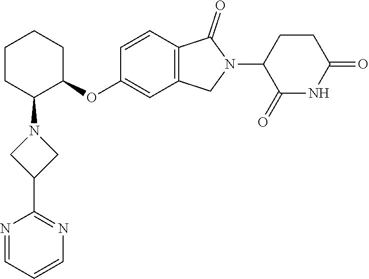 [SMILES: O=C1CCC(N2Cc3cc(O[C@@H]4CCCC[C@@H]4N4CC(c5ncccn5)C4)ccc3C2=O)C(=O)N1]

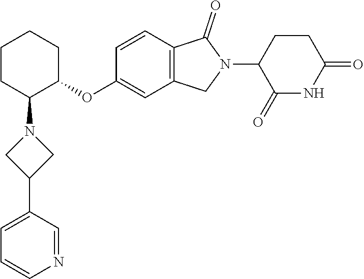 [SMILES: O=C1CCC(N2Cc3cc(O[C@H]4CCCC[C@@H]4N4CC(c5cccnc5)C4)ccc3C2=O)C(=O)N1]

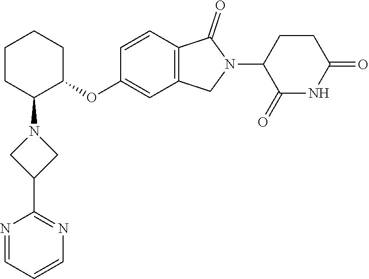 [SMILES: O=C1CCC(N2Cc3cc(O[C@H]4CCCC[C@@H]4N4CC(c5ncccn5)C4)ccc3C2=O)C(=O)N1]

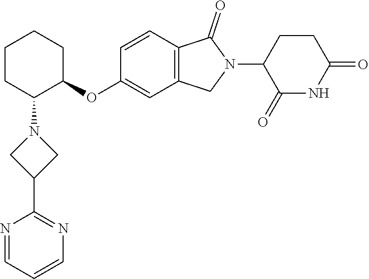 [SMILES: O=C1CCC(N2Cc3cc(O[C@@H]4CCCC[C@H]4N4CC(c5ncccn5)C4)ccc3C2=O)C(=O)N1]

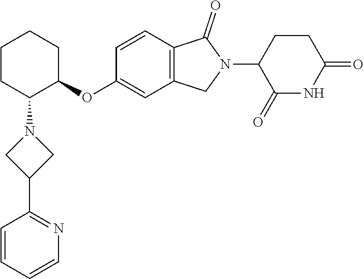 [SMILES: O=C1CCC(N2Cc3cc(O[C@@H]4CCCC[C@H]4N4CC(c5ccccn5)C4)ccc3C2=O)C(=O)N1]

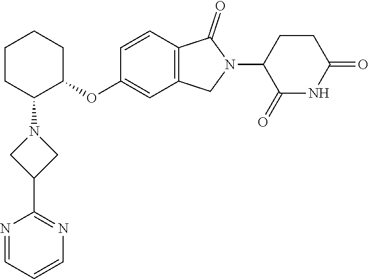 [SMILES: O=C1CCC(N2Cc3cc(O[C@H]4CCCC[C@H]4N4CC(c5ncccn5)C4)ccc3C2=O)C(=O)N1]

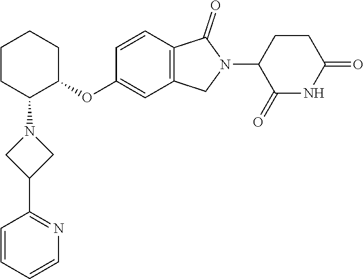 [SMILES: O=C1CCC(N2Cc3cc(O[C@H]4CCCC[C@H]4N4CC(c5ccccn5)C4)ccc3C2=O)C(=O)N1]

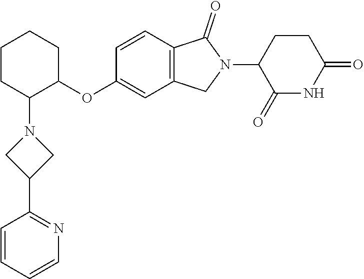 [SMILES: O=C1CCC(N2Cc3cc(OC4CCCCC4N4CC(c5ccccn5)C4)ccc3C2=O)C(=O)N1]

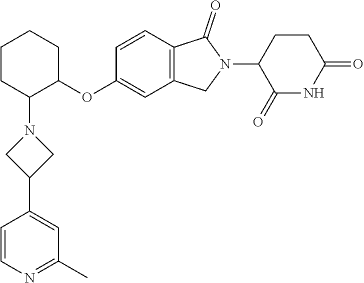 [SMILES: Cc1cc(C2CN(C3CCCCC3Oc3ccc4c(c3)CN(C3CCC(=O)NC3=O)C4=O)C2)ccn1]

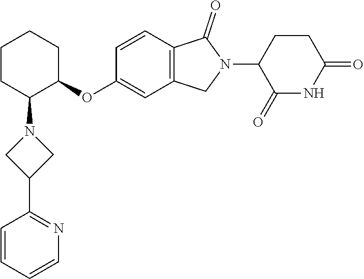 [SMILES: O=C1CCC(N2Cc3cc(O[C@@H]4CCCC[C@@H]4N4CC(c5ccccn5)C4)ccc3C2=O)C(=O)N1]

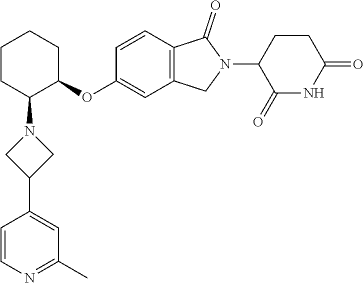 [SMILES: Cc1cc(C2CN([C@H]3CCCC[C@H]3Oc3ccc4c(c3)CN(C3CCC(=O)NC3=O)C4=O)C2)ccn1]

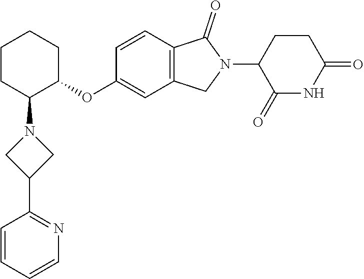 [SMILES: O=C1CCC(N2Cc3cc(O[C@H]4CCCC[C@@H]4N4CC(c5ccccn5)C4)ccc3C2=O)C(=O)N1]

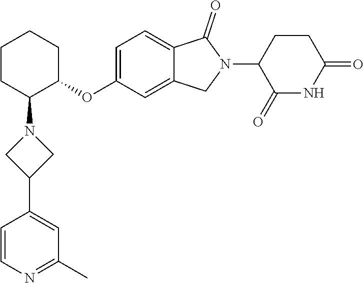 [SMILES: Cc1cc(C2CN([C@H]3CCCC[C@@H]3Oc3ccc4c(c3)CN(C3CCC(=O)NC3=O)C4=O)C2)ccn1]

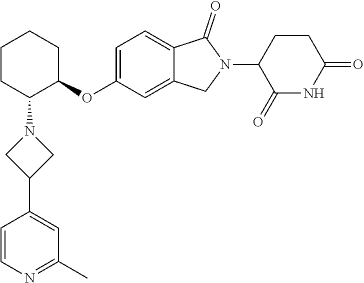 [SMILES: Cc1cc(C2CN([C@@H]3CCCC[C@H]3Oc3ccc4c(c3)CN(C3CCC(=O)NC3=O)C4=O)C2)ccn1]

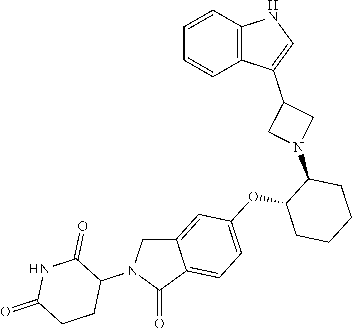 [SMILES: O=C1CCC(N2Cc3cc(O[C@H]4CCCC[C@@H]4N4CC(c5c[nH]c6ccccc56)C4)ccc3C2=O)C(=O)N1]

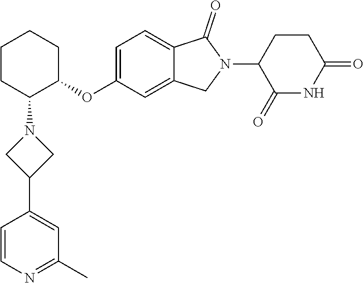 [SMILES: Cc1cc(C2CN([C@@H]3CCCC[C@@H]3Oc3ccc4c(c3)CN(C3CCC(=O)NC3=O)C4=O)C2)ccn1]

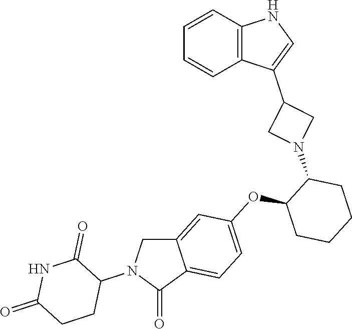 [SMILES: O=C1CCC(N2Cc3cc(O[C@@H]4CCCC[C@H]4N4CC(c5c[nH]c6ccccc56)C4)ccc3C2=O)C(=O)N1]

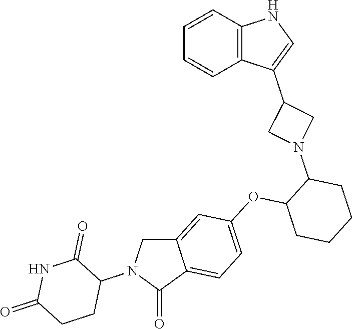 [SMILES: O=C1CCC(N2Cc3cc(OC4CCCCC4N4CC(c5c[nH]c6ccccc56)C4)ccc3C2=O)C(=O)N1]

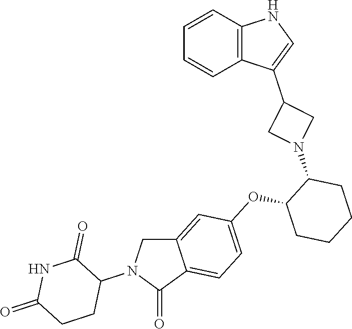 [SMILES: O=C1CCC(N2Cc3cc(O[C@H]4CCCC[C@H]4N4CC(c5c[nH]c6ccccc56)C4)ccc3C2=O)C(=O)N1]

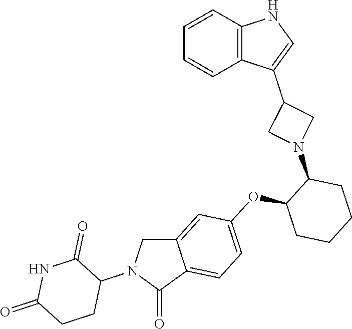 [SMILES: O=C1CCC(N2Cc3cc(O[C@@H]4CCCC[C@@H]4N4CC(c5c[nH]c6ccccc56)C4)ccc3C2=O)C(=O)N1]

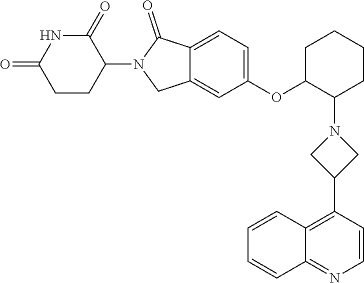 [SMILES: O=C1CCC(N2Cc3cc(OC4CCCCC4N4CC(c5ccnc6ccccc56)C4)ccc3C2=O)C(=O)N1]

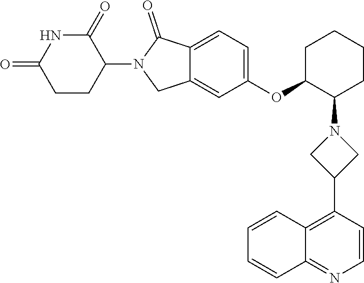 [SMILES: O=C1CCC(N2Cc3cc(O[C@H]4CCCC[C@H]4N4CC(c5ccnc6ccccc56)C4)ccc3C2=O)C(=O)N1]

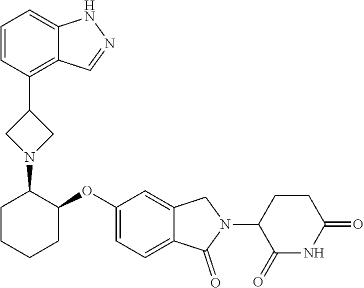 [SMILES: O=C1CCC(N2Cc3cc(O[C@H]4CCCC[C@H]4N4CC(c5cccc6[nH]ncc56)C4)ccc3C2=O)C(=O)N1]

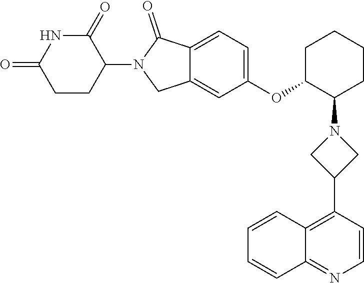 [SMILES: O=C1CCC(N2Cc3cc(O[C@@H]4CCCC[C@H]4N4CC(c5ccnc6ccccc56)C4)ccc3C2=O)C(=O)N1]

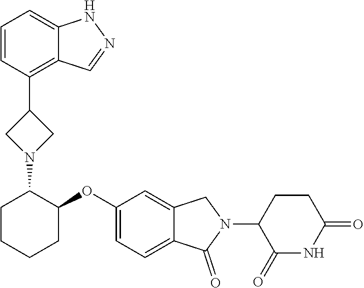 [SMILES: O=C1CCC(N2Cc3cc(O[C@H]4CCCC[C@@H]4N4CC(c5cccc6[nH]ncc56)C4)ccc3C2=O)C(=O)N1]

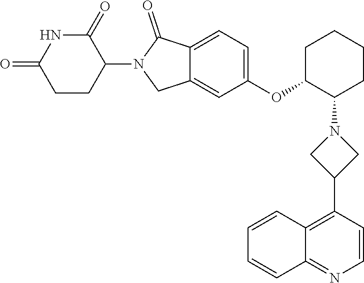 [SMILES: O=C1CCC(N2Cc3cc(O[C@@H]4CCCC[C@@H]4N4CC(c5ccnc6ccccc56)C4)ccc3C2=O)C(=O)N1]

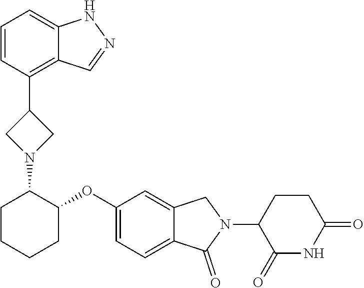 [SMILES: O=C1CCC(N2Cc3cc(O[C@@H]4CCCC[C@@H]4N4CC(c5cccc6[nH]ncc56)C4)ccc3C2=O)C(=O)N1]

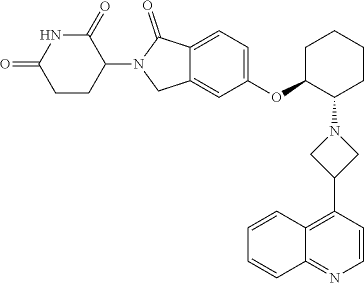 [SMILES: O=C1CCC(N2Cc3cc(O[C@H]4CCCC[C@@H]4N4CC(c5ccnc6ccccc56)C4)ccc3C2=O)C(=O)N1]

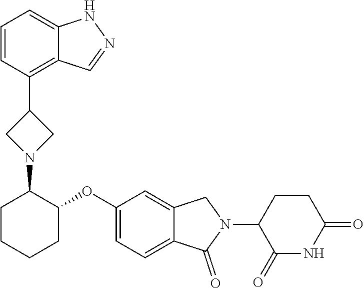 [SMILES: O=C1CCC(N2Cc3cc(O[C@@H]4CCCC[C@H]4N4CC(c5cccc6[nH]ncc56)C4)ccc3C2=O)C(=O)N1]

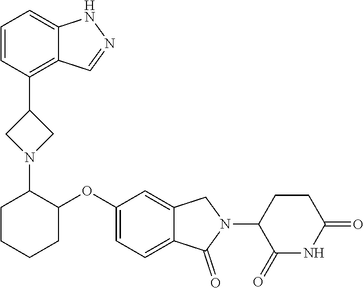 [SMILES: O=C1CCC(N2Cc3cc(OC4CCCCC4N4CC(c5cccc6[nH]ncc56)C4)ccc3C2=O)C(=O)N1]

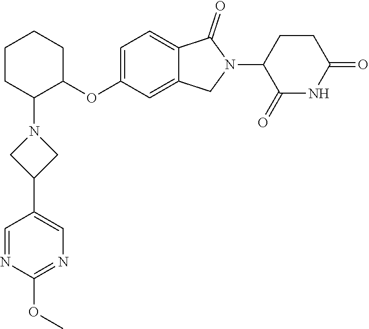 [SMILES: COc1ncc(C2CN(C3CCCCC3Oc3ccc4c(c3)CN(C3CCC(=O)NC3=O)C4=O)C2)cn1]

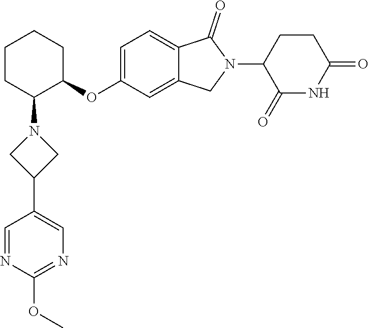 [SMILES: COc1ncc(C2CN([C@H]3CCCC[C@H]3Oc3ccc4c(c3)CN(C3CCC(=O)NC3=O)C4=O)C2)cn1]

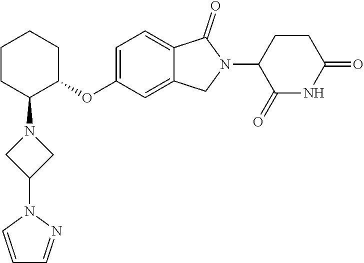 [SMILES: O=C1CCC(N2Cc3cc(O[C@H]4CCCC[C@@H]4N4CC(n5cccn5)C4)ccc3C2=O)C(=O)N1]

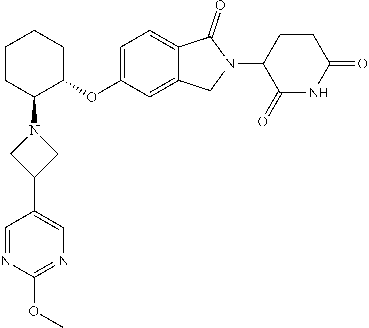 [SMILES: COc1ncc(C2CN([C@H]3CCCC[C@@H]3Oc3ccc4c(c3)CN(C3CCC(=O)NC3=O)C4=O)C2)cn1]

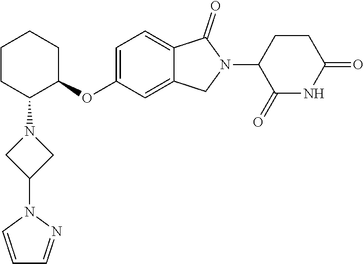 [SMILES: O=C1CCC(N2Cc3cc(O[C@@H]4CCCC[C@H]4N4CC(n5cccn5)C4)ccc3C2=O)C(=O)N1]

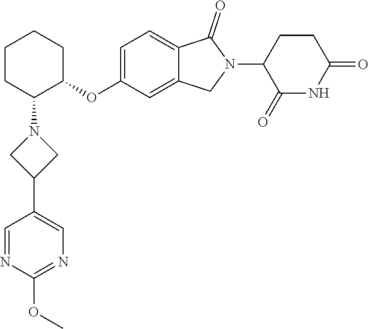 [SMILES: COc1ncc(C2CN([C@@H]3CCCC[C@@H]3Oc3ccc4c(c3)CN(C3CCC(=O)NC3=O)C4=O)C2)cn1]

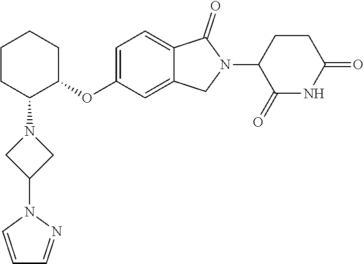 [SMILES: O=C1CCC(N2Cc3cc(O[C@H]4CCCC[C@H]4N4CC(n5cccn5)C4)ccc3C2=O)C(=O)N1]

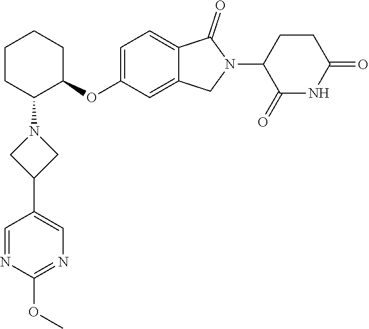 [SMILES: COc1ncc(C2CN([C@@H]3CCCC[C@H]3Oc3ccc4c(c3)CN(C3CCC(=O)NC3=O)C4=O)C2)cn1]

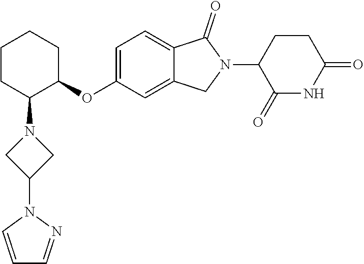 [SMILES: O=C1CCC(N2Cc3cc(O[C@@H]4CCCC[C@@H]4N4CC(n5cccn5)C4)ccc3C2=O)C(=O)N1]

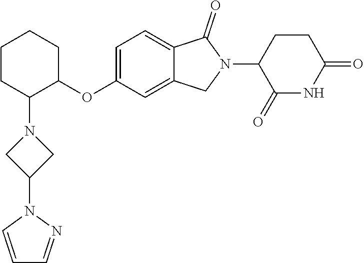 [SMILES: O=C1CCC(N2Cc3cc(OC4CCCCC4N4CC(n5cccn5)C4)ccc3C2=O)C(=O)N1]

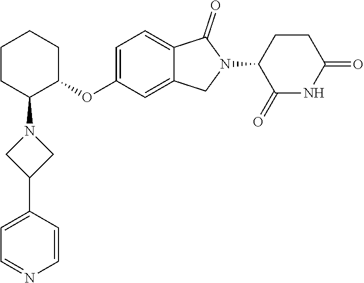 [SMILES: O=C1CC[C@@H](N2Cc3cc(O[C@H]4CCCC[C@@H]4N4CC(c5ccncc5)C4)ccc3C2=O)C(=O)N1]

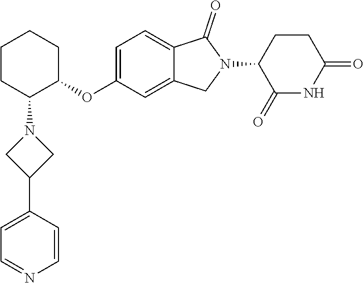 [SMILES: O=C1CC[C@@H](N2Cc3cc(O[C@H]4CCCC[C@H]4N4CC(c5ccncc5)C4)ccc3C2=O)C(=O)N1]

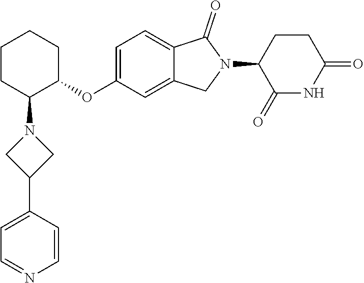 [SMILES: O=C1CC[C@H](N2Cc3cc(O[C@H]4CCCC[C@@H]4N4CC(c5ccncc5)C4)ccc3C2=O)C(=O)N1]

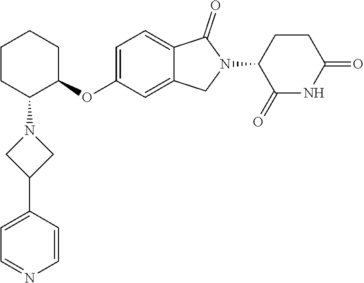 [SMILES: O=C1CC[C@@H](N2Cc3cc(O[C@@H]4CCCC[C@H]4N4CC(c5ccncc5)C4)ccc3C2=O)C(=O)N1]

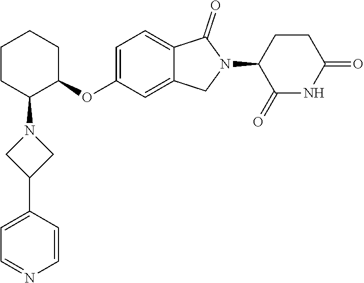 [SMILES: O=C1CC[C@H](N2Cc3cc(O[C@@H]4CCCC[C@@H]4N4CC(c5ccncc5)C4)ccc3C2=O)C(=O)N1]

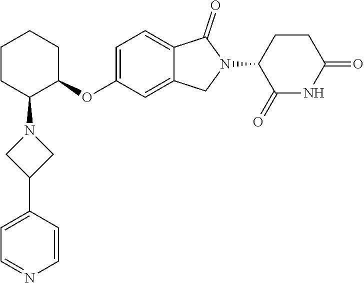 [SMILES: O=C1CC[C@@H](N2Cc3cc(O[C@@H]4CCCC[C@@H]4N4CC(c5ccncc5)C4)ccc3C2=O)C(=O)N1]

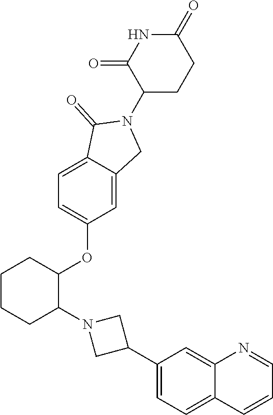 [SMILES: O=C1CCC(N2Cc3cc(OC4CCCCC4N4CC(c5ccc6cccnc6c5)C4)ccc3C2=O)C(=O)N1]

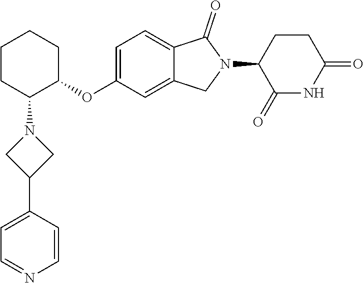 [SMILES: O=C1CC[C@H](N2Cc3cc(O[C@H]4CCCC[C@H]4N4CC(c5ccncc5)C4)ccc3C2=O)C(=O)N1]

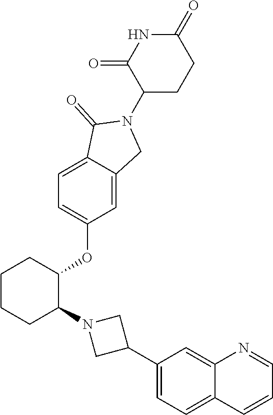 [SMILES: O=C1CCC(N2Cc3cc(O[C@H]4CCCC[C@@H]4N4CC(c5ccc6cccnc6c5)C4)ccc3C2=O)C(=O)N1]

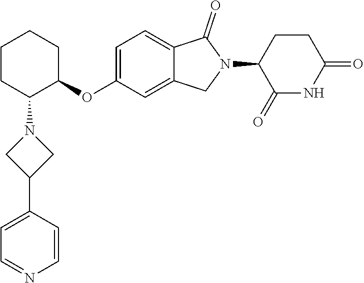 [SMILES: O=C1CC[C@H](N2Cc3cc(O[C@@H]4CCCC[C@H]4N4CC(c5ccncc5)C4)ccc3C2=O)C(=O)N1]

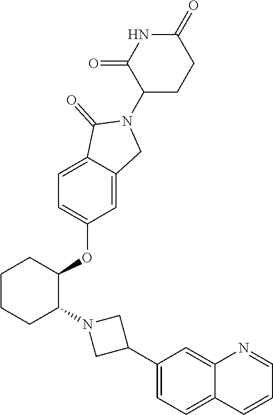 [SMILES: O=C1CCC(N2Cc3cc(O[C@@H]4CCCC[C@H]4N4CC(c5ccc6cccnc6c5)C4)ccc3C2=O)C(=O)N1]

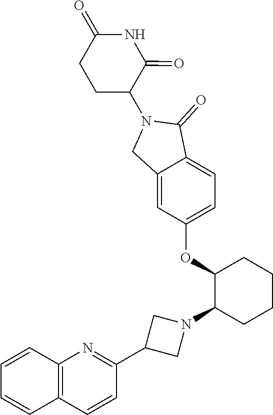 [SMILES: O=C1CCC(N2Cc3cc(O[C@H]4CCCC[C@H]4N4CC(c5ccc6ccccc6n5)C4)ccc3C2=O)C(=O)N1]

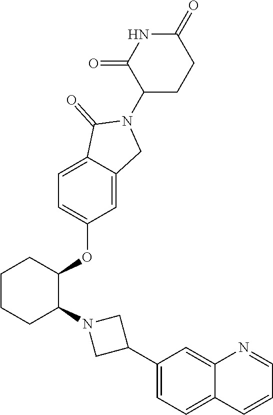 [SMILES: O=C1CCC(N2Cc3cc(O[C@@H]4CCCC[C@@H]4N4CC(c5ccc6cccnc6c5)C4)ccc3C2=O)C(=O)N1]

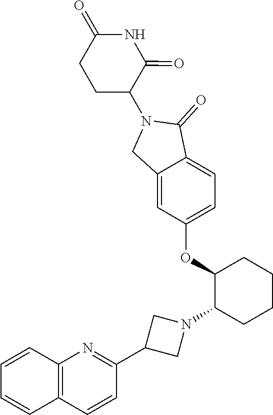 [SMILES: O=C1CCC(N2Cc3cc(O[C@H]4CCCC[C@@H]4N4CC(c5ccc6ccccc6n5)C4)ccc3C2=O)C(=O)N1]

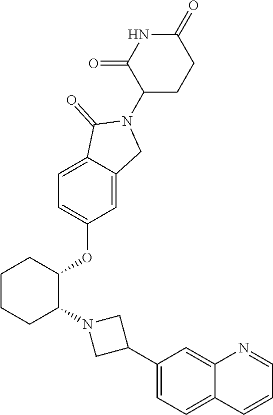 [SMILES: O=C1CCC(N2Cc3cc(O[C@H]4CCCC[C@H]4N4CC(c5ccc6cccnc6c5)C4)ccc3C2=O)C(=O)N1]

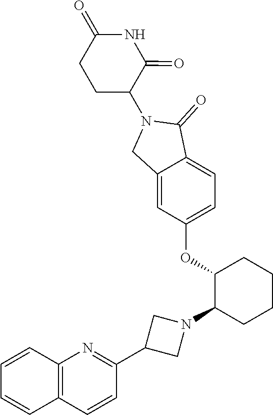 [SMILES: O=C1CCC(N2Cc3cc(O[C@@H]4CCCC[C@H]4N4CC(c5ccc6ccccc6n5)C4)ccc3C2=O)C(=O)N1]

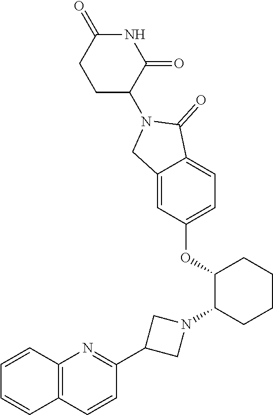 [SMILES: O=C1CCC(N2Cc3cc(O[C@@H]4CCCC[C@@H]4N4CC(c5ccc6ccccc6n5)C4)ccc3C2=O)C(=O)N1]

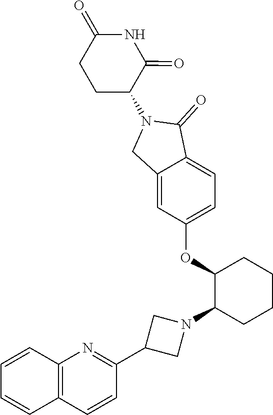 [SMILES: O=C1CC[C@@H](N2Cc3cc(O[C@H]4CCCC[C@H]4N4CC(c5ccc6ccccc6n5)C4)ccc3C2=O)C(=O)N1]

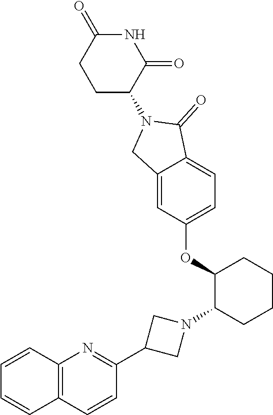 [SMILES: O=C1CC[C@@H](N2Cc3cc(O[C@H]4CCCC[C@@H]4N4CC(c5ccc6ccccc6n5)C4)ccc3C2=O)C(=O)N1]

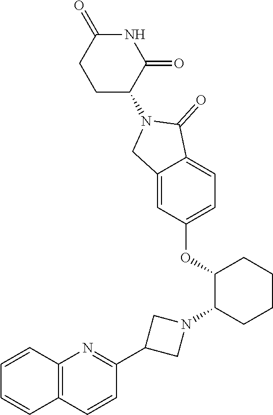 [SMILES: O=C1CC[C@@H](N2Cc3cc(O[C@@H]4CCCC[C@@H]4N4CC(c5ccc6ccccc6n5)C4)ccc3C2=O)C(=O)N1]

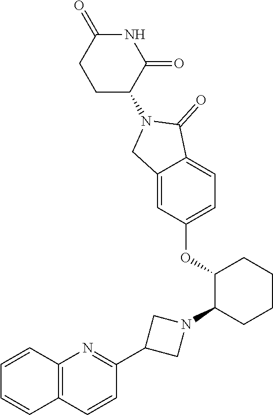 [SMILES: O=C1CC[C@@H](N2Cc3cc(O[C@@H]4CCCC[C@H]4N4CC(c5ccc6ccccc6n5)C4)ccc3C2=O)C(=O)N1]

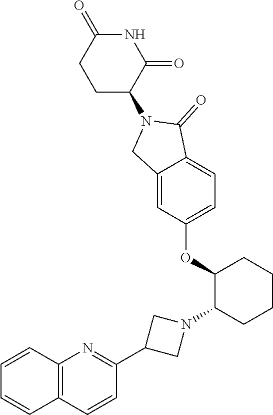 [SMILES: O=C1CC[C@H](N2Cc3cc(O[C@H]4CCCC[C@@H]4N4CC(c5ccc6ccccc6n5)C4)ccc3C2=O)C(=O)N1]

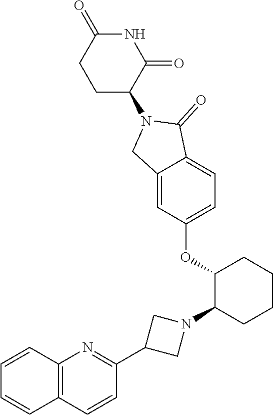 [SMILES: O=C1CC[C@H](N2Cc3cc(O[C@@H]4CCCC[C@H]4N4CC(c5ccc6ccccc6n5)C4)ccc3C2=O)C(=O)N1]

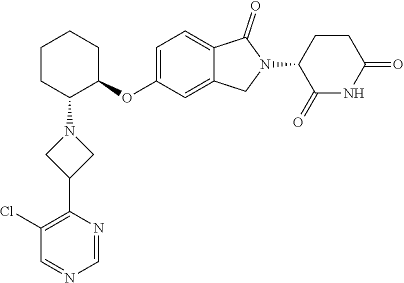 [SMILES: O=C1CC[C@@H](N2Cc3cc(O[C@@H]4CCCC[C@H]4N4CC(c5ncncc5Cl)C4)ccc3C2=O)C(=O)N1]

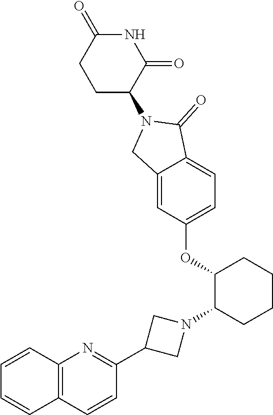 [SMILES: O=C1CC[C@H](N2Cc3cc(O[C@@H]4CCCC[C@@H]4N4CC(c5ccc6ccccc6n5)C4)ccc3C2=O)C(=O)N1]

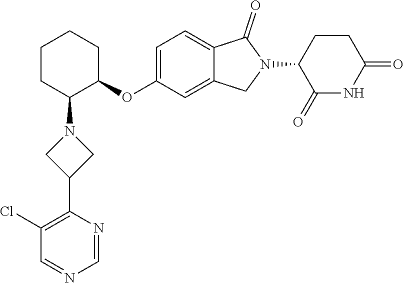 [SMILES: O=C1CC[C@@H](N2Cc3cc(O[C@@H]4CCCC[C@@H]4N4CC(c5ncncc5Cl)C4)ccc3C2=O)C(=O)N1]

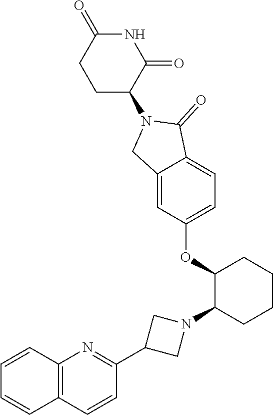 [SMILES: O=C1CC[C@H](N2Cc3cc(O[C@H]4CCCC[C@H]4N4CC(c5ccc6ccccc6n5)C4)ccc3C2=O)C(=O)N1]

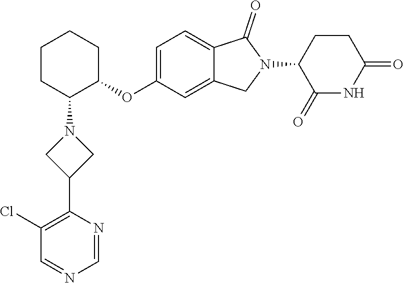 [SMILES: O=C1CC[C@@H](N2Cc3cc(O[C@H]4CCCC[C@H]4N4CC(c5ncncc5Cl)C4)ccc3C2=O)C(=O)N1]

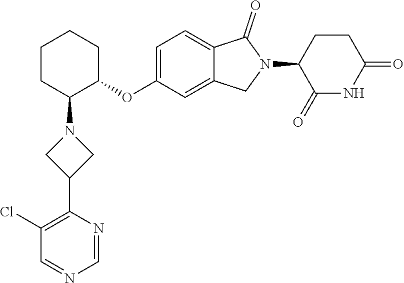 [SMILES: O=C1CC[C@H](N2Cc3cc(O[C@H]4CCCC[C@@H]4N4CC(c5ncncc5Cl)C4)ccc3C2=O)C(=O)N1]

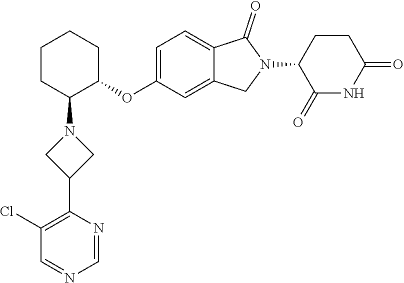 [SMILES: O=C1CC[C@@H](N2Cc3cc(O[C@H]4CCCC[C@@H]4N4CC(c5ncncc5Cl)C4)ccc3C2=O)C(=O)N1]

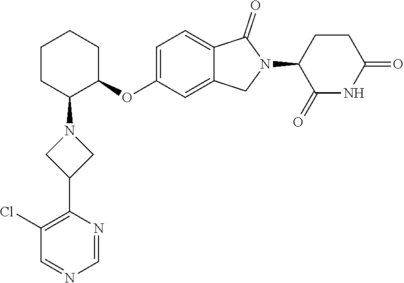 [SMILES: O=C1CC[C@H](N2Cc3cc(O[C@@H]4CCCC[C@@H]4N4CC(c5ncncc5Cl)C4)ccc3C2=O)C(=O)N1]

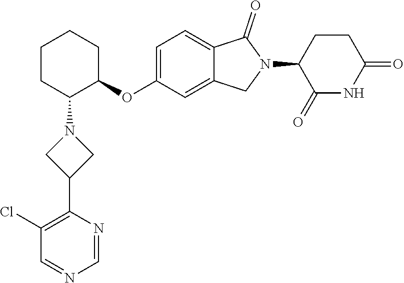 [SMILES: O=C1CC[C@H](N2Cc3cc(O[C@@H]4CCCC[C@H]4N4CC(c5ncncc5Cl)C4)ccc3C2=O)C(=O)N1]

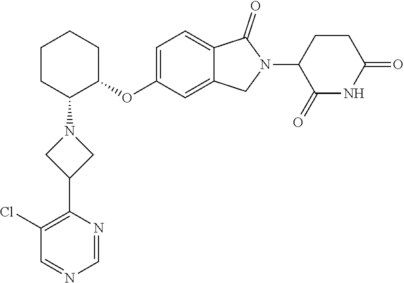 [SMILES: O=C1CCC(N2Cc3cc(O[C@H]4CCCC[C@H]4N4CC(c5ncncc5Cl)C4)ccc3C2=O)C(=O)N1]

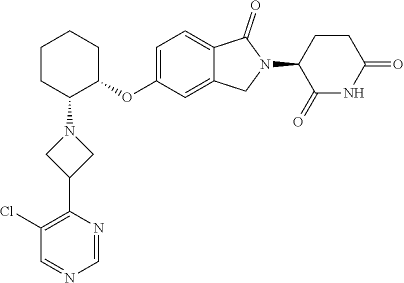 [SMILES: O=C1CC[C@H](N2Cc3cc(O[C@H]4CCCC[C@H]4N4CC(c5ncncc5Cl)C4)ccc3C2=O)C(=O)N1]

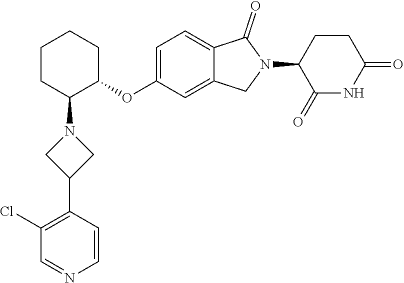 [SMILES: O=C1CC[C@H](N2Cc3cc(O[C@H]4CCCC[C@@H]4N4CC(c5ccncc5Cl)C4)ccc3C2=O)C(=O)N1]

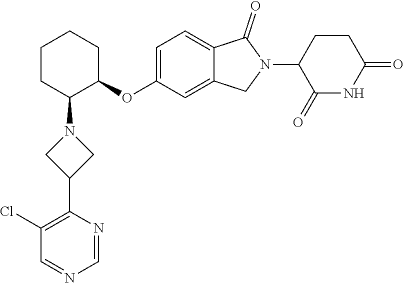 [SMILES: O=C1CCC(N2Cc3cc(O[C@@H]4CCCC[C@@H]4N4CC(c5ncncc5Cl)C4)ccc3C2=O)C(=O)N1]

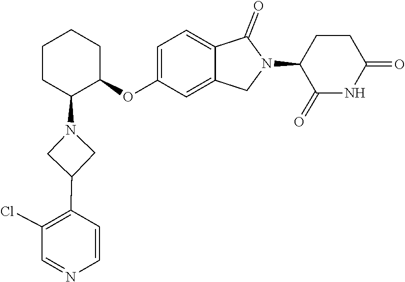 [SMILES: O=C1CC[C@H](N2Cc3cc(O[C@@H]4CCCC[C@@H]4N4CC(c5ccncc5Cl)C4)ccc3C2=O)C(=O)N1]

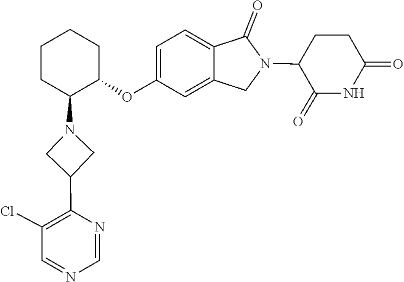 [SMILES: O=C1CCC(N2Cc3cc(O[C@H]4CCCC[C@@H]4N4CC(c5ncncc5Cl)C4)ccc3C2=O)C(=O)N1]

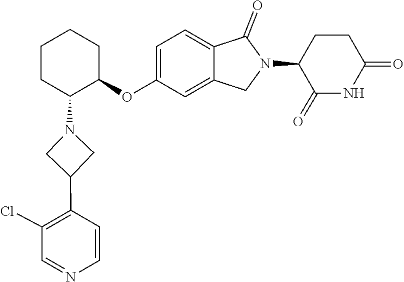 [SMILES: O=C1CC[C@H](N2Cc3cc(O[C@@H]4CCCC[C@H]4N4CC(c5ccncc5Cl)C4)ccc3C2=O)C(=O)N1]

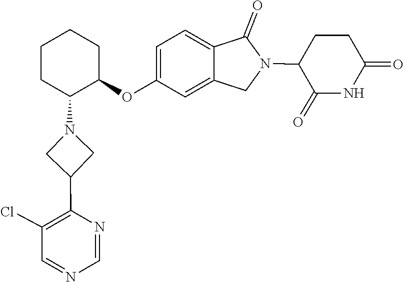 [SMILES: O=C1CCC(N2Cc3cc(O[C@@H]4CCCC[C@H]4N4CC(c5ncncc5Cl)C4)ccc3C2=O)C(=O)N1]

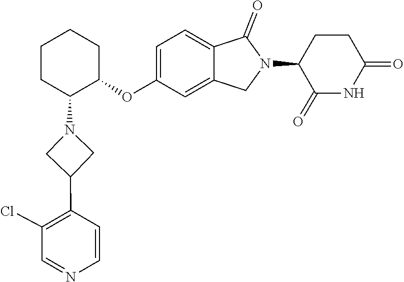 [SMILES: O=C1CC[C@H](N2Cc3cc(O[C@H]4CCCC[C@H]4N4CC(c5ccncc5Cl)C4)ccc3C2=O)C(=O)N1]

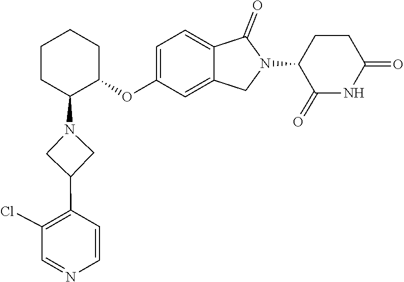 [SMILES: O=C1CC[C@@H](N2Cc3cc(O[C@H]4CCCC[C@@H]4N4CC(c5ccncc5Cl)C4)ccc3C2=O)C(=O)N1]

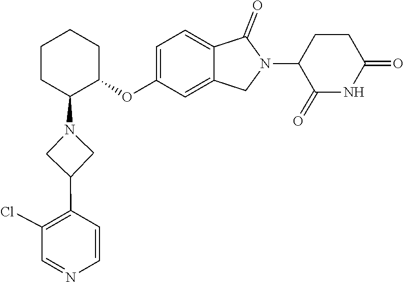 [SMILES: O=C1CCC(N2Cc3cc(O[C@H]4CCCC[C@@H]4N4CC(c5ccncc5Cl)C4)ccc3C2=O)C(=O)N1]

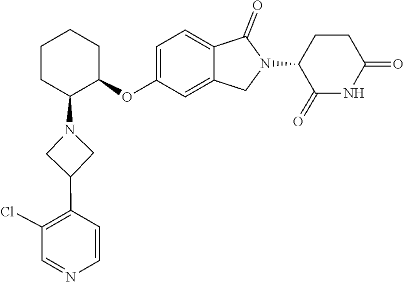 [SMILES: O=C1CC[C@@H](N2Cc3cc(O[C@@H]4CCCC[C@@H]4N4CC(c5ccncc5Cl)C4)ccc3C2=O)C(=O)N1]

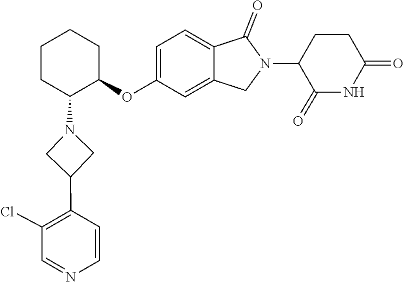 [SMILES: O=C1CCC(N2Cc3cc(O[C@@H]4CCCC[C@H]4N4CC(c5ccncc5Cl)C4)ccc3C2=O)C(=O)N1]

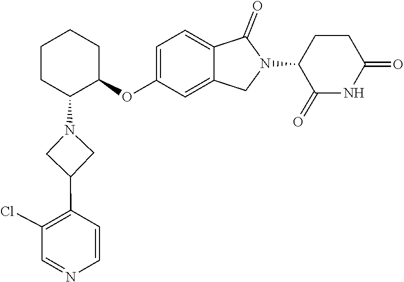 [SMILES: O=C1CC[C@@H](N2Cc3cc(O[C@@H]4CCCC[C@H]4N4CC(c5ccncc5Cl)C4)ccc3C2=O)C(=O)N1]

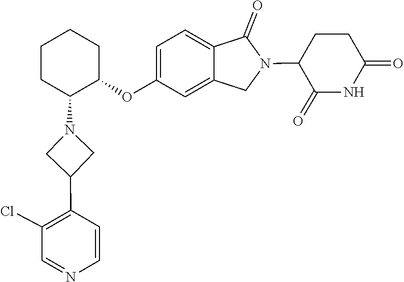 [SMILES: O=C1CCC(N2Cc3cc(O[C@H]4CCCC[C@H]4N4CC(c5ccncc5Cl)C4)ccc3C2=O)C(=O)N1]

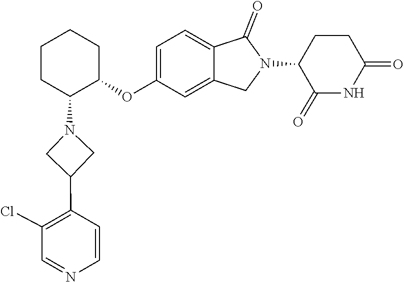 [SMILES: O=C1CC[C@@H](N2Cc3cc(O[C@H]4CCCC[C@H]4N4CC(c5ccncc5Cl)C4)ccc3C2=O)C(=O)N1]

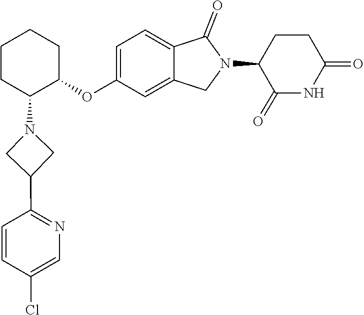 [SMILES: O=C1CC[C@H](N2Cc3cc(O[C@H]4CCCC[C@H]4N4CC(c5ccc(Cl)cn5)C4)ccc3C2=O)C(=O)N1]

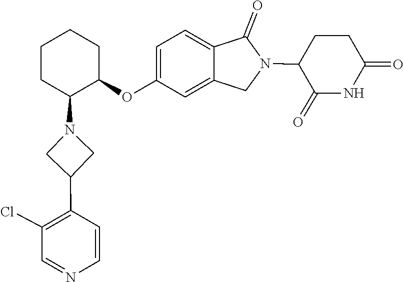 [SMILES: O=C1CCC(N2Cc3cc(O[C@@H]4CCCC[C@@H]4N4CC(c5ccncc5Cl)C4)ccc3C2=O)C(=O)N1]

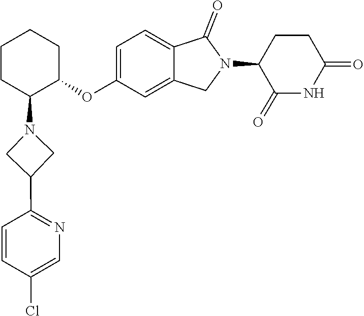 [SMILES: O=C1CC[C@H](N2Cc3cc(O[C@H]4CCCC[C@@H]4N4CC(c5ccc(Cl)cn5)C4)ccc3C2=O)C(=O)N1]

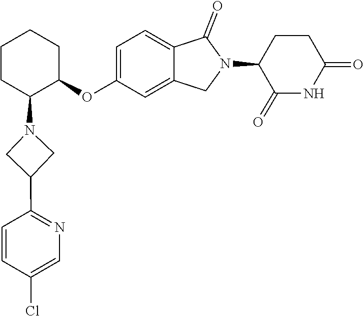 [SMILES: O=C1CC[C@H](N2Cc3cc(O[C@@H]4CCCC[C@@H]4N4CC(c5ccc(Cl)cn5)C4)ccc3C2=O)C(=O)N1]

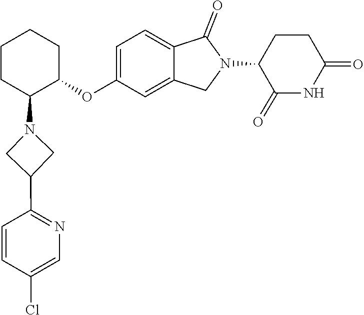 [SMILES: O=C1CC[C@@H](N2Cc3cc(O[C@H]4CCCC[C@@H]4N4CC(c5ccc(Cl)cn5)C4)ccc3C2=O)C(=O)N1]

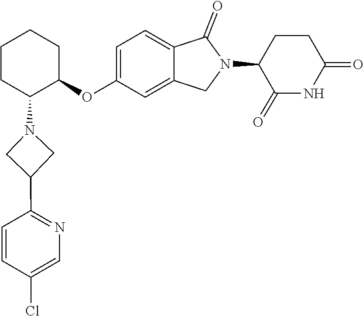 [SMILES: O=C1CC[C@H](N2Cc3cc(O[C@@H]4CCCC[C@H]4N4CC(c5ccc(Cl)cn5)C4)ccc3C2=O)C(=O)N1]

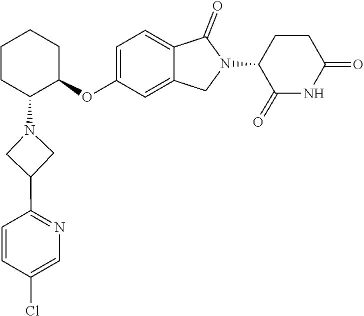 [SMILES: O=C1CC[C@@H](N2Cc3cc(O[C@@H]4CCCC[C@H]4N4CC(c5ccc(Cl)cn5)C4)ccc3C2=O)C(=O)N1]

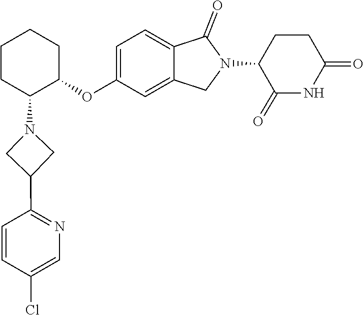 [SMILES: O=C1CC[C@@H](N2Cc3cc(O[C@H]4CCCC[C@H]4N4CC(c5ccc(Cl)cn5)C4)ccc3C2=O)C(=O)N1]

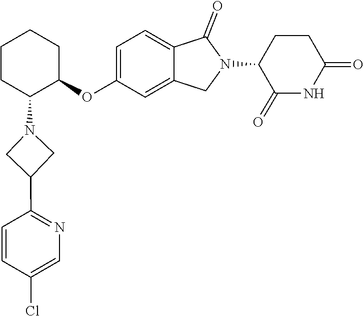 [SMILES: O=C1CC[C@@H](N2Cc3cc(O[C@@H]4CCCC[C@H]4N4CC(c5ccc(Cl)cn5)C4)ccc3C2=O)C(=O)N1]

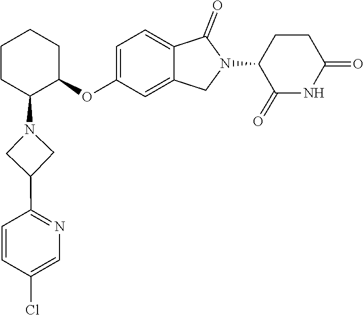 [SMILES: O=C1CC[C@@H](N2Cc3cc(O[C@@H]4CCCC[C@@H]4N4CC(c5ccc(Cl)cn5)C4)ccc3C2=O)C(=O)N1]

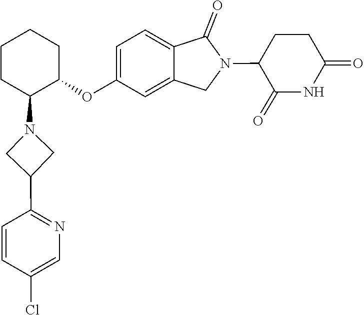 [SMILES: O=C1CCC(N2Cc3cc(O[C@H]4CCCC[C@@H]4N4CC(c5ccc(Cl)cn5)C4)ccc3C2=O)C(=O)N1]

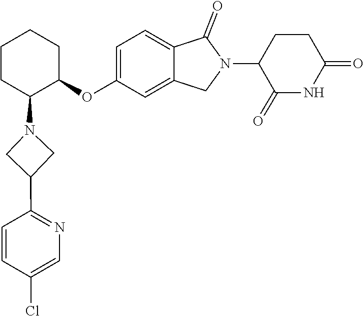 [SMILES: O=C1CCC(N2Cc3cc(O[C@@H]4CCCC[C@@H]4N4CC(c5ccc(Cl)cn5)C4)ccc3C2=O)C(=O)N1]

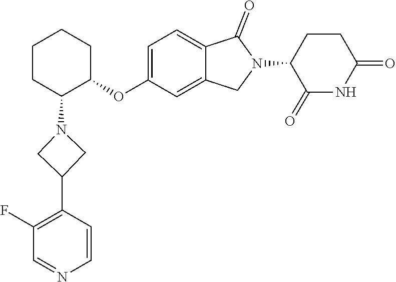 [SMILES: O=C1CC[C@@H](N2Cc3cc(O[C@H]4CCCC[C@H]4N4CC(c5ccncc5F)C4)ccc3C2=O)C(=O)N1]

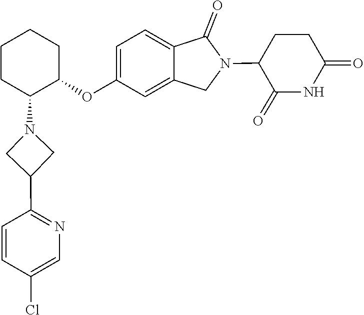 [SMILES: O=C1CCC(N2Cc3cc(O[C@H]4CCCC[C@H]4N4CC(c5ccc(Cl)cn5)C4)ccc3C2=O)C(=O)N1]

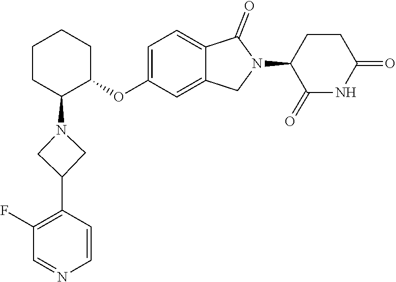 [SMILES: O=C1CC[C@H](N2Cc3cc(O[C@H]4CCCC[C@@H]4N4CC(c5ccncc5F)C4)ccc3C2=O)C(=O)N1]

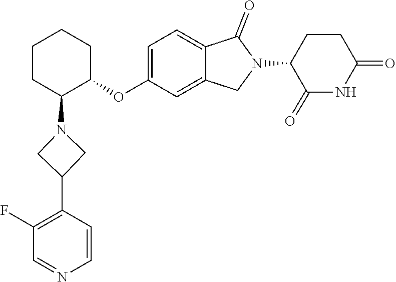 [SMILES: O=C1CC[C@@H](N2Cc3cc(O[C@H]4CCCC[C@@H]4N4CC(c5ccncc5F)C4)ccc3C2=O)C(=O)N1]

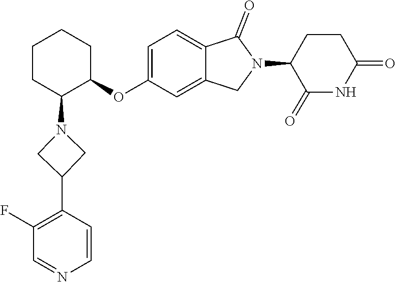 [SMILES: O=C1CC[C@H](N2Cc3cc(O[C@@H]4CCCC[C@@H]4N4CC(c5ccncc5F)C4)ccc3C2=O)C(=O)N1]

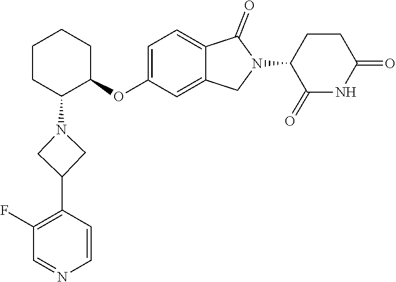 [SMILES: O=C1CC[C@@H](N2Cc3cc(O[C@@H]4CCCC[C@H]4N4CC(c5ccncc5F)C4)ccc3C2=O)C(=O)N1]

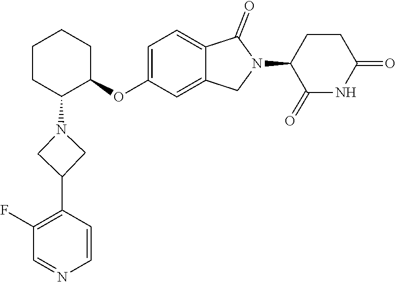 [SMILES: O=C1CC[C@H](N2Cc3cc(O[C@@H]4CCCC[C@H]4N4CC(c5ccncc5F)C4)ccc3C2=O)C(=O)N1]

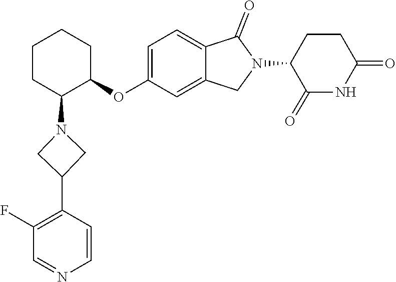 [SMILES: O=C1CC[C@@H](N2Cc3cc(O[C@@H]4CCCC[C@@H]4N4CC(c5ccncc5F)C4)ccc3C2=O)C(=O)N1]

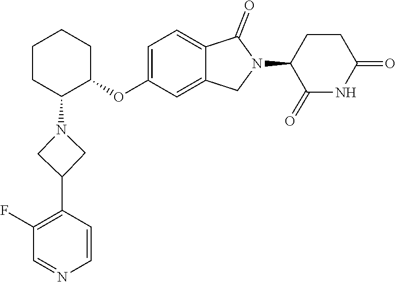 [SMILES: O=C1CC[C@H](N2Cc3cc(O[C@H]4CCCC[C@H]4N4CC(c5ccncc5F)C4)ccc3C2=O)C(=O)N1]

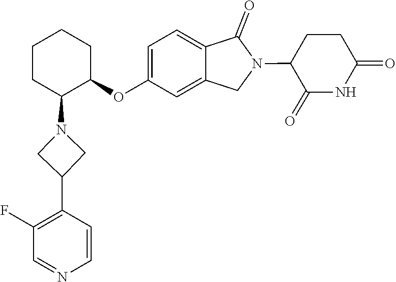 [SMILES: O=C1CCC(N2Cc3cc(O[C@@H]4CCCC[C@@H]4N4CC(c5ccncc5F)C4)ccc3C2=O)C(=O)N1]

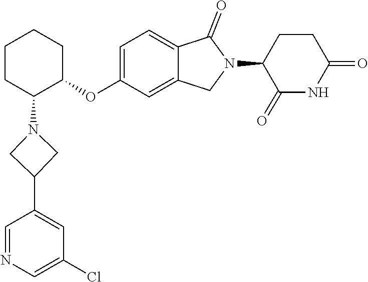 [SMILES: O=C1CC[C@H](N2Cc3cc(O[C@H]4CCCC[C@H]4N4CC(c5cncc(Cl)c5)C4)ccc3C2=O)C(=O)N1]

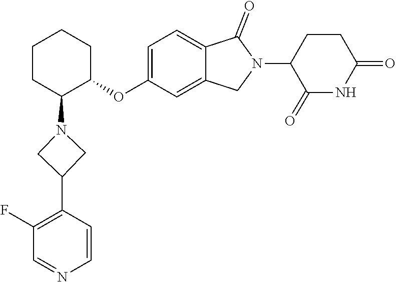 [SMILES: O=C1CCC(N2Cc3cc(O[C@H]4CCCC[C@@H]4N4CC(c5ccncc5F)C4)ccc3C2=O)C(=O)N1]

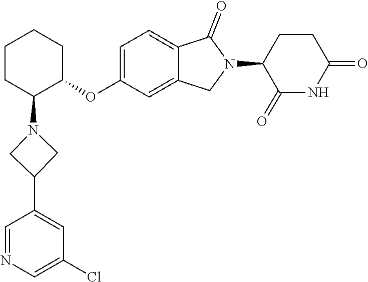 [SMILES: O=C1CC[C@H](N2Cc3cc(O[C@H]4CCCC[C@@H]4N4CC(c5cncc(Cl)c5)C4)ccc3C2=O)C(=O)N1]

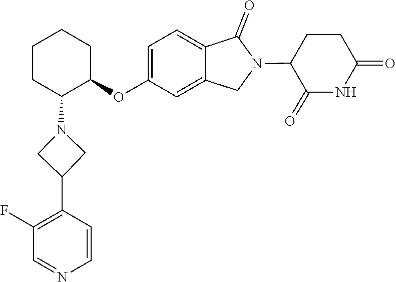 [SMILES: O=C1CCC(N2Cc3cc(O[C@@H]4CCCC[C@H]4N4CC(c5ccncc5F)C4)ccc3C2=O)C(=O)N1]

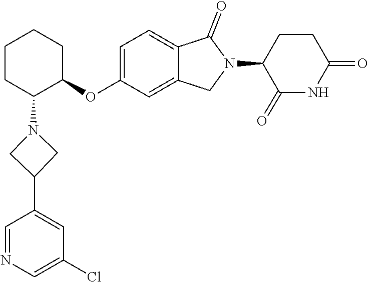 [SMILES: O=C1CC[C@H](N2Cc3cc(O[C@@H]4CCCC[C@H]4N4CC(c5cncc(Cl)c5)C4)ccc3C2=O)C(=O)N1]

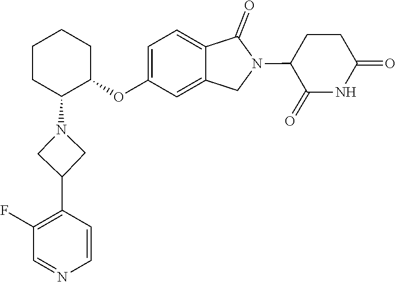 [SMILES: O=C1CCC(N2Cc3cc(O[C@H]4CCCC[C@H]4N4CC(c5ccncc5F)C4)ccc3C2=O)C(=O)N1]

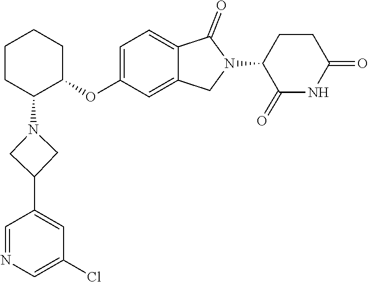 [SMILES: O=C1CC[C@@H](N2Cc3cc(O[C@H]4CCCC[C@H]4N4CC(c5cncc(Cl)c5)C4)ccc3C2=O)C(=O)N1]

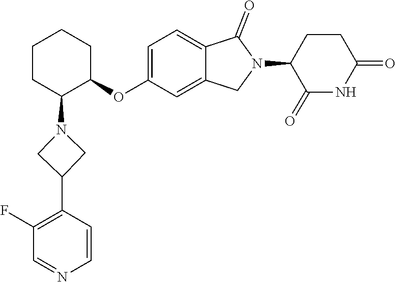 [SMILES: O=C1CC[C@H](N2Cc3cc(O[C@@H]4CCCC[C@@H]4N4CC(c5ccncc5F)C4)ccc3C2=O)C(=O)N1]

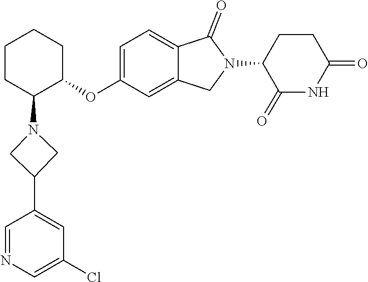 [SMILES: O=C1CC[C@@H](N2Cc3cc(O[C@H]4CCCC[C@@H]4N4CC(c5cncc(Cl)c5)C4)ccc3C2=O)C(=O)N1]

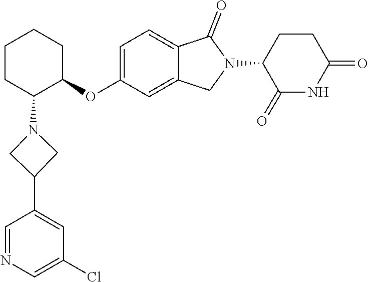 [SMILES: O=C1CC[C@@H](N2Cc3cc(O[C@@H]4CCCC[C@H]4N4CC(c5cncc(Cl)c5)C4)ccc3C2=O)C(=O)N1]

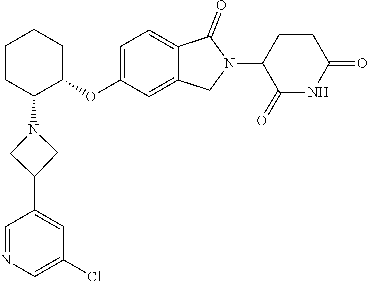 [SMILES: O=C1CCC(N2Cc3cc(O[C@H]4CCCC[C@H]4N4CC(c5cncc(Cl)c5)C4)ccc3C2=O)C(=O)N1]

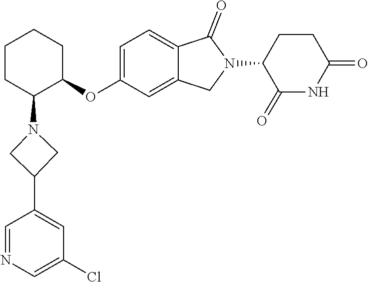 [SMILES: O=C1CC[C@@H](N2Cc3cc(O[C@@H]4CCCC[C@@H]4N4CC(c5cncc(Cl)c5)C4)ccc3C2=O)C(=O)N1]

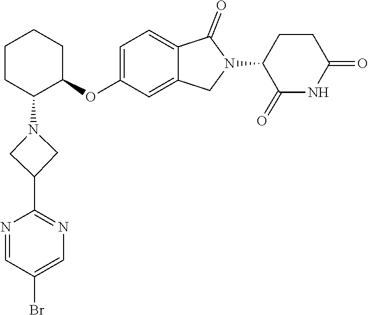 [SMILES: O=C1CC[C@@H](N2Cc3cc(O[C@@H]4CCCC[C@H]4N4CC(c5ncc(Br)cn5)C4)ccc3C2=O)C(=O)N1]

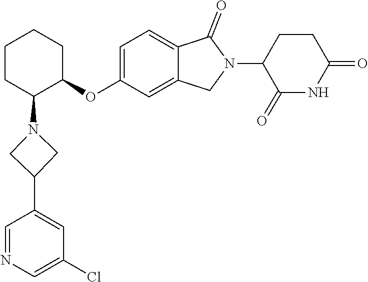 [SMILES: O=C1CCC(N2Cc3cc(O[C@@H]4CCCC[C@@H]4N4CC(c5cncc(Cl)c5)C4)ccc3C2=O)C(=O)N1]

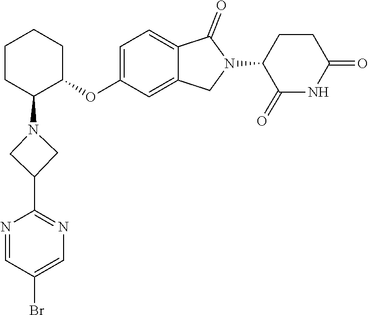 [SMILES: O=C1CC[C@@H](N2Cc3cc(O[C@H]4CCCC[C@@H]4N4CC(c5ncc(Br)cn5)C4)ccc3C2=O)C(=O)N1]

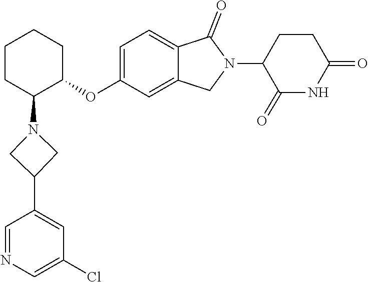 [SMILES: O=C1CCC(N2Cc3cc(O[C@H]4CCCC[C@@H]4N4CC(c5cncc(Cl)c5)C4)ccc3C2=O)C(=O)N1]

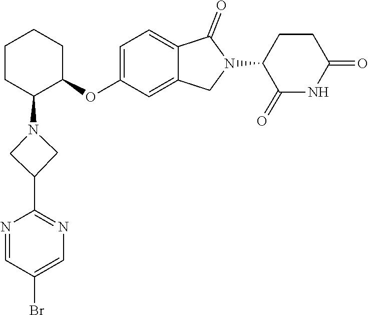 [SMILES: O=C1CC[C@@H](N2Cc3cc(O[C@@H]4CCCC[C@@H]4N4CC(c5ncc(Br)cn5)C4)ccc3C2=O)C(=O)N1]

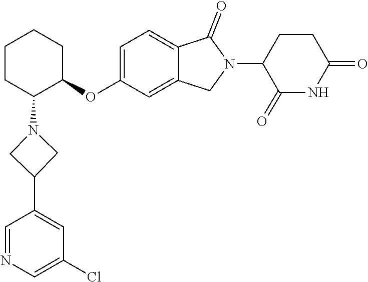 [SMILES: O=C1CCC(N2Cc3cc(O[C@@H]4CCCC[C@H]4N4CC(c5cncc(Cl)c5)C4)ccc3C2=O)C(=O)N1]

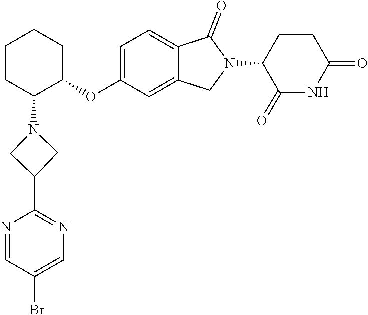 [SMILES: O=C1CC[C@@H](N2Cc3cc(O[C@H]4CCCC[C@H]4N4CC(c5ncc(Br)cn5)C4)ccc3C2=O)C(=O)N1]

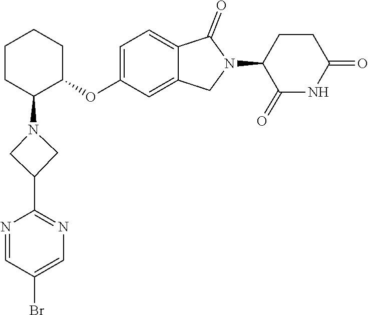 [SMILES: O=C1CC[C@H](N2Cc3cc(O[C@H]4CCCC[C@@H]4N4CC(c5ncc(Br)cn5)C4)ccc3C2=O)C(=O)N1]

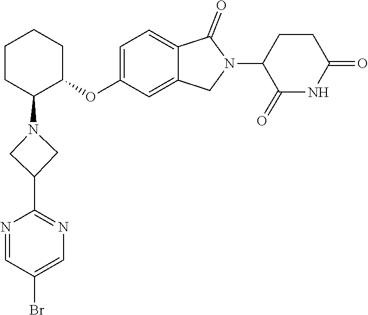 [SMILES: O=C1CCC(N2Cc3cc(O[C@H]4CCCC[C@@H]4N4CC(c5ncc(Br)cn5)C4)ccc3C2=O)C(=O)N1]

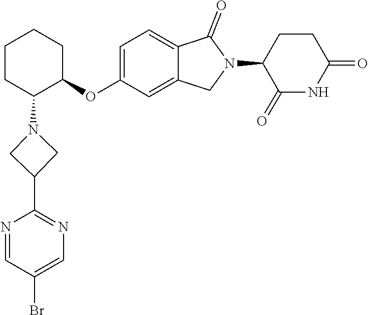 [SMILES: O=C1CC[C@H](N2Cc3cc(O[C@@H]4CCCC[C@H]4N4CC(c5ncc(Br)cn5)C4)ccc3C2=O)C(=O)N1]

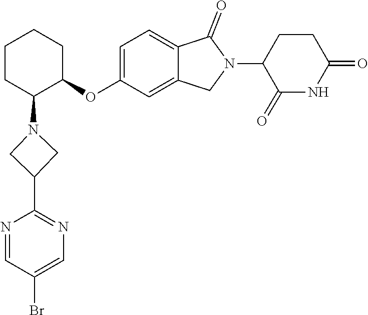 [SMILES: O=C1CCC(N2Cc3cc(O[C@@H]4CCCC[C@@H]4N4CC(c5ncc(Br)cn5)C4)ccc3C2=O)C(=O)N1]

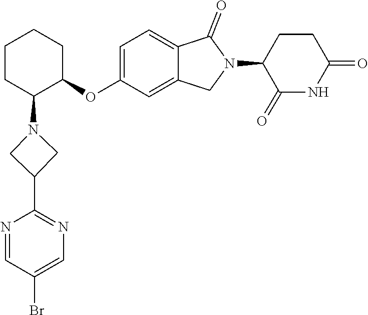 [SMILES: O=C1CC[C@H](N2Cc3cc(O[C@@H]4CCCC[C@@H]4N4CC(c5ncc(Br)cn5)C4)ccc3C2=O)C(=O)N1]

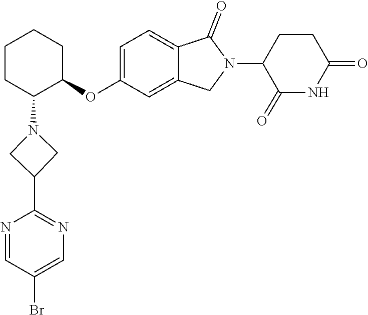 [SMILES: O=C1CCC(N2Cc3cc(O[C@@H]4CCCC[C@H]4N4CC(c5ncc(Br)cn5)C4)ccc3C2=O)C(=O)N1]

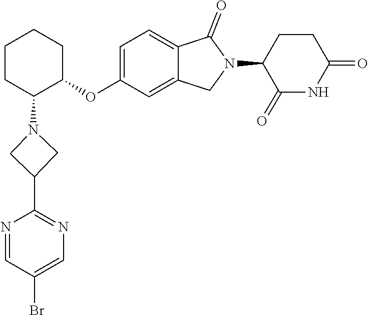 [SMILES: O=C1CC[C@H](N2Cc3cc(O[C@H]4CCCC[C@H]4N4CC(c5ncc(Br)cn5)C4)ccc3C2=O)C(=O)N1]

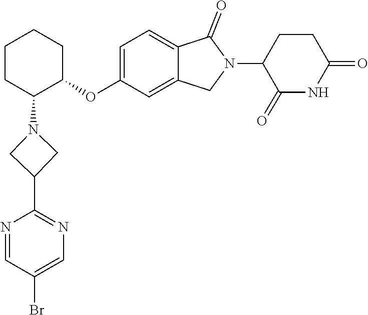 [SMILES: O=C1CCC(N2Cc3cc(O[C@H]4CCCC[C@H]4N4CC(c5ncc(Br)cn5)C4)ccc3C2=O)C(=O)N1]

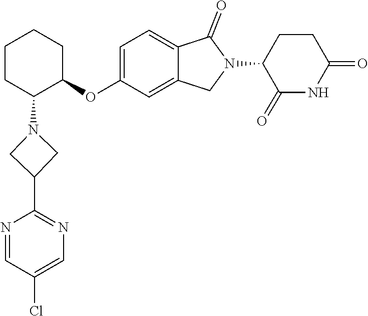 [SMILES: O=C1CC[C@@H](N2Cc3cc(O[C@@H]4CCCC[C@H]4N4CC(c5ncc(Cl)cn5)C4)ccc3C2=O)C(=O)N1]

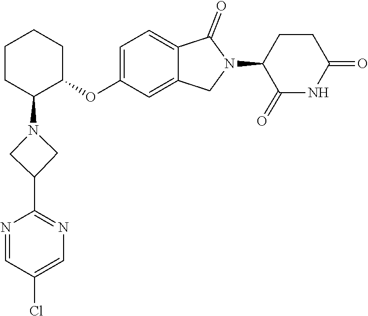 [SMILES: O=C1CC[C@H](N2Cc3cc(O[C@H]4CCCC[C@@H]4N4CC(c5ncc(Cl)cn5)C4)ccc3C2=O)C(=O)N1]

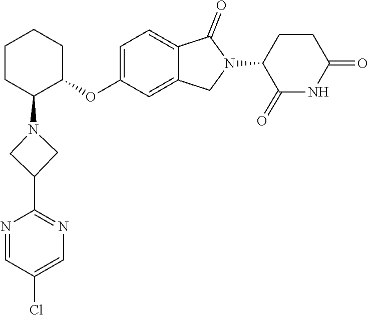 [SMILES: O=C1CC[C@@H](N2Cc3cc(O[C@H]4CCCC[C@@H]4N4CC(c5ncc(Cl)cn5)C4)ccc3C2=O)C(=O)N1]

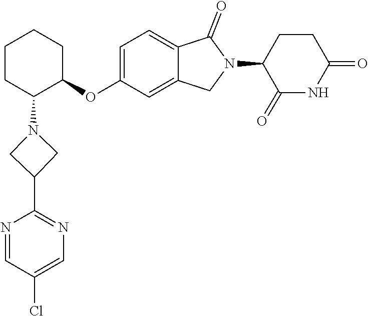 [SMILES: O=C1CC[C@H](N2Cc3cc(O[C@@H]4CCCC[C@H]4N4CC(c5ncc(Cl)cn5)C4)ccc3C2=O)C(=O)N1]

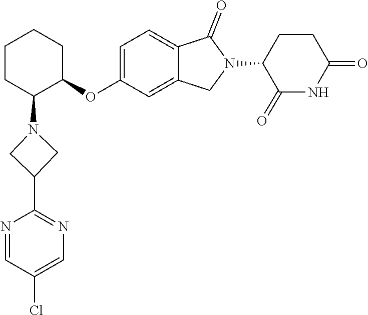 [SMILES: O=C1CC[C@@H](N2Cc3cc(O[C@@H]4CCCC[C@@H]4N4CC(c5ncc(Cl)cn5)C4)ccc3C2=O)C(=O)N1]

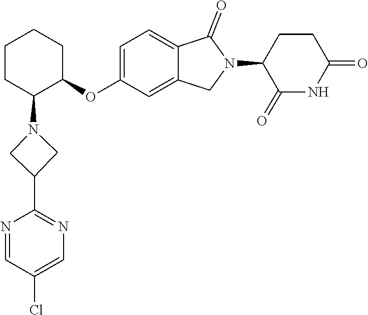 [SMILES: O=C1CC[C@H](N2Cc3cc(O[C@@H]4CCCC[C@@H]4N4CC(c5ncc(Cl)cn5)C4)ccc3C2=O)C(=O)N1]

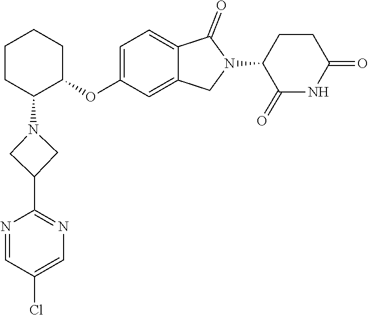 [SMILES: O=C1CC[C@@H](N2Cc3cc(O[C@H]4CCCC[C@H]4N4CC(c5ncc(Cl)cn5)C4)ccc3C2=O)C(=O)N1]

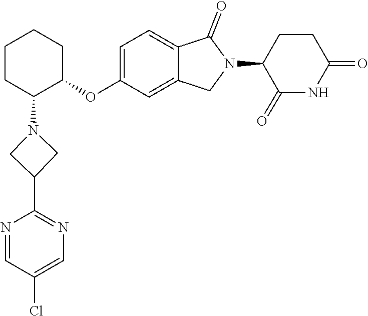 [SMILES: O=C1CC[C@H](N2Cc3cc(O[C@H]4CCCC[C@H]4N4CC(c5ncc(Cl)cn5)C4)ccc3C2=O)C(=O)N1]

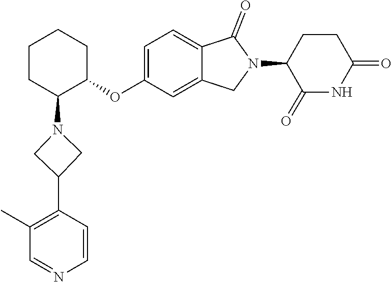 [SMILES: Cc1cnccc1C1CN([C@H]2CCCC[C@@H]2Oc2ccc3c(c2)CN([C@H]2CCC(=O)NC2=O)C3=O)C1]

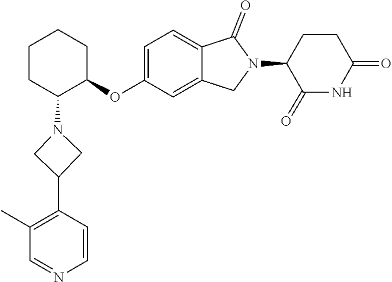 [SMILES: Cc1cnccc1C1CN([C@@H]2CCCC[C@H]2Oc2ccc3c(c2)CN([C@H]2CCC(=O)NC2=O)C3=O)C1]

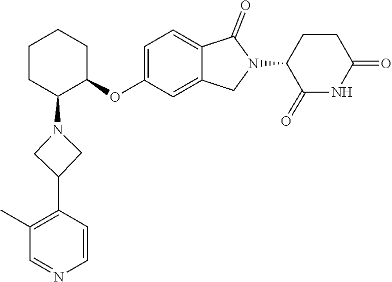 [SMILES: Cc1cnccc1C1CN([C@H]2CCCC[C@H]2Oc2ccc3c(c2)CN([C@@H]2CCC(=O)NC2=O)C3=O)C1]

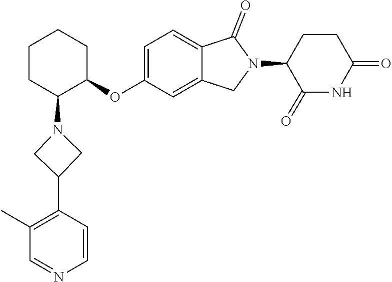 [SMILES: Cc1cnccc1C1CN([C@H]2CCCC[C@H]2Oc2ccc3c(c2)CN([C@H]2CCC(=O)NC2=O)C3=O)C1]

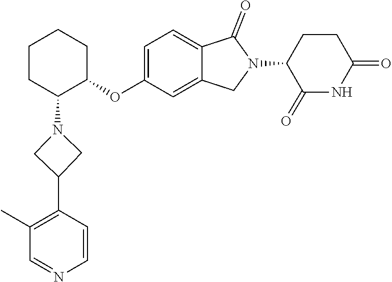 [SMILES: Cc1cnccc1C1CN([C@@H]2CCCC[C@@H]2Oc2ccc3c(c2)CN([C@@H]2CCC(=O)NC2=O)C3=O)C1]

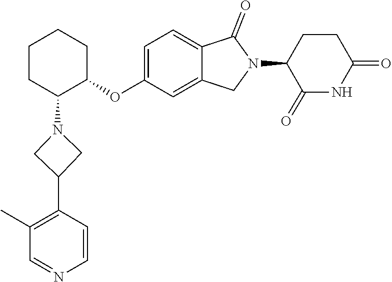 [SMILES: Cc1cnccc1C1CN([C@@H]2CCCC[C@@H]2Oc2ccc3c(c2)CN([C@H]2CCC(=O)NC2=O)C3=O)C1]

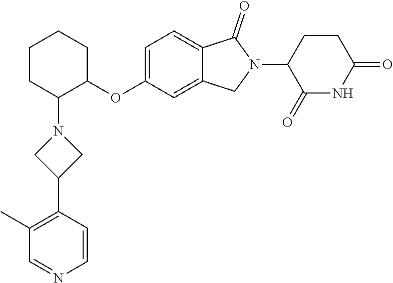 [SMILES: Cc1cnccc1C1CN(C2CCCCC2Oc2ccc3c(c2)CN(C2CCC(=O)NC2=O)C3=O)C1]

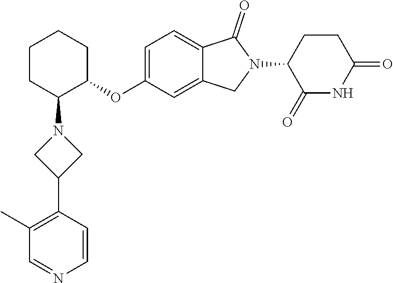 [SMILES: Cc1cnccc1C1CN([C@H]2CCCC[C@@H]2Oc2ccc3c(c2)CN([C@@H]2CCC(=O)NC2=O)C3=O)C1]

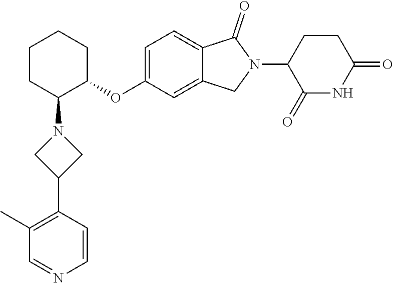 [SMILES: Cc1cnccc1C1CN([C@H]2CCCC[C@@H]2Oc2ccc3c(c2)CN(C2CCC(=O)NC2=O)C3=O)C1]

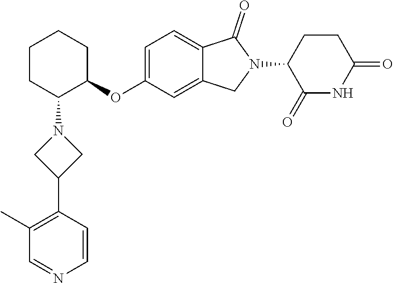 [SMILES: Cc1cnccc1C1CN([C@@H]2CCCC[C@H]2Oc2ccc3c(c2)CN([C@@H]2CCC(=O)NC2=O)C3=O)C1]

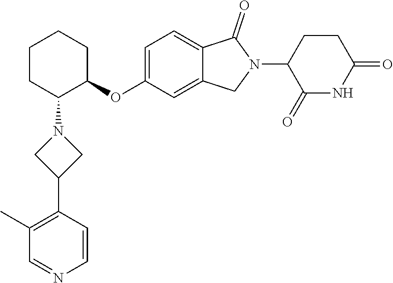 [SMILES: Cc1cnccc1C1CN([C@@H]2CCCC[C@H]2Oc2ccc3c(c2)CN(C2CCC(=O)NC2=O)C3=O)C1]

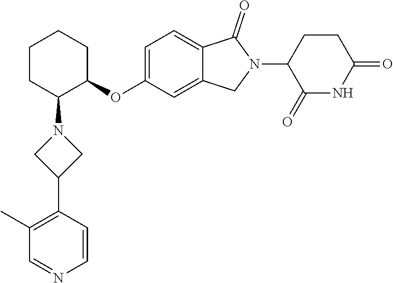 [SMILES: Cc1cnccc1C1CN([C@H]2CCCC[C@H]2Oc2ccc3c(c2)CN(C2CCC(=O)NC2=O)C3=O)C1]

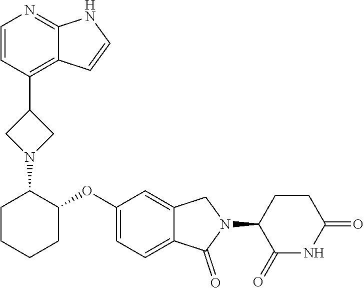 [SMILES: O=C1CC[C@H](N2Cc3cc(O[C@@H]4CCCC[C@@H]4N4CC(c5ccnc6[nH]ccc56)C4)ccc3C2=O)C(=O)N1]

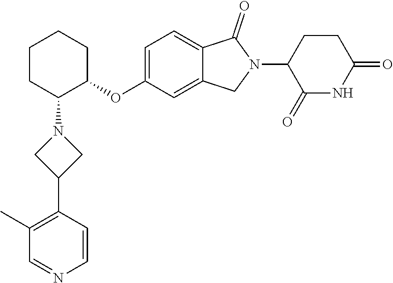 [SMILES: Cc1cnccc1C1CN([C@@H]2CCCC[C@@H]2Oc2ccc3c(c2)CN(C2CCC(=O)NC2=O)C3=O)C1]

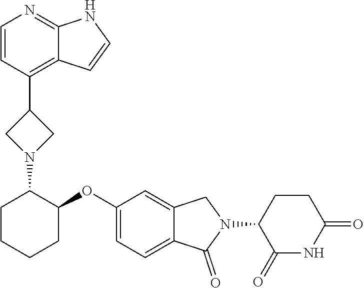 [SMILES: O=C1CC[C@@H](N2Cc3cc(O[C@H]4CCCC[C@@H]4N4CC(c5ccnc6[nH]ccc56)C4)ccc3C2=O)C(=O)N1]

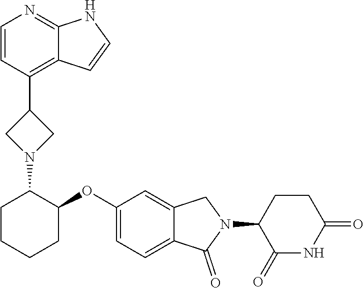 [SMILES: O=C1CC[C@H](N2Cc3cc(O[C@H]4CCCC[C@@H]4N4CC(c5ccnc6[nH]ccc56)C4)ccc3C2=O)C(=O)N1]

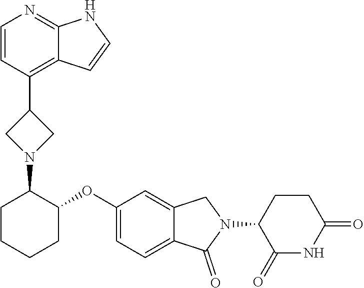 [SMILES: O=C1CC[C@@H](N2Cc3cc(O[C@@H]4CCCC[C@H]4N4CC(c5ccnc6[nH]ccc56)C4)ccc3C2=O)C(=O)N1]

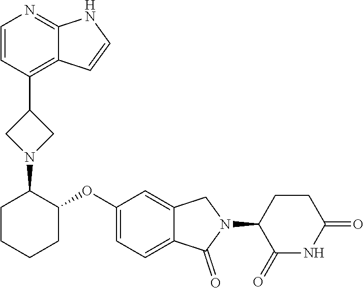 [SMILES: O=C1CC[C@H](N2Cc3cc(O[C@@H]4CCCC[C@H]4N4CC(c5ccnc6[nH]ccc56)C4)ccc3C2=O)C(=O)N1]

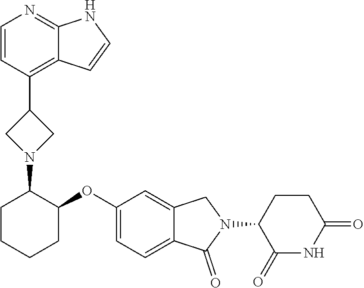 [SMILES: O=C1CC[C@@H](N2Cc3cc(O[C@H]4CCCC[C@H]4N4CC(c5ccnc6[nH]ccc56)C4)ccc3C2=O)C(=O)N1]

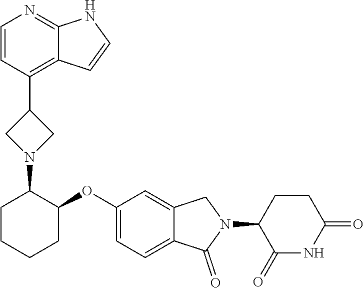 [SMILES: O=C1CC[C@H](N2Cc3cc(O[C@H]4CCCC[C@H]4N4CC(c5ccnc6[nH]ccc56)C4)ccc3C2=O)C(=O)N1]

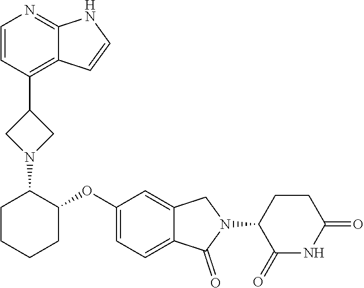 [SMILES: O=C1CC[C@@H](N2Cc3cc(O[C@@H]4CCCC[C@@H]4N4CC(c5ccnc6[nH]ccc56)C4)ccc3C2=O)C(=O)N1]

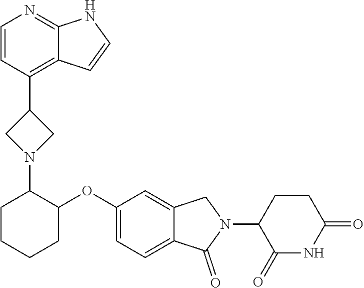 [SMILES: O=C1CCC(N2Cc3cc(OC4CCCCC4N4CC(c5ccnc6[nH]ccc56)C4)ccc3C2=O)C(=O)N1]

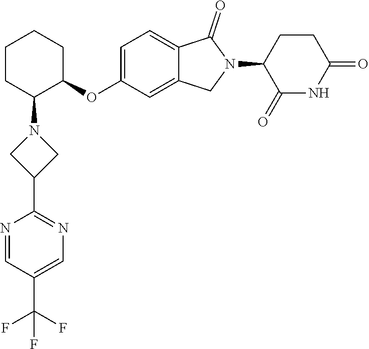 [SMILES: O=C1CC[C@H](N2Cc3cc(O[C@@H]4CCCC[C@@H]4N4CC(c5ncc(C(F)(F)F)cn5)C4)ccc3C2=O)C(=O)N1]

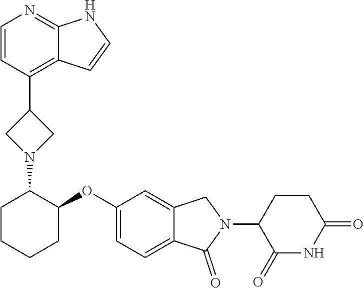 [SMILES: O=C1CCC(N2Cc3cc(O[C@H]4CCCC[C@@H]4N4CC(c5ccnc6[nH]ccc56)C4)ccc3C2=O)C(=O)N1]

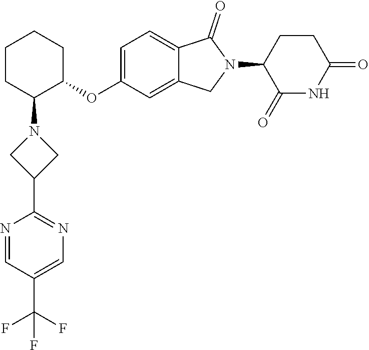 [SMILES: O=C1CC[C@H](N2Cc3cc(O[C@H]4CCCC[C@@H]4N4CC(c5ncc(C(F)(F)F)cn5)C4)ccc3C2=O)C(=O)N1]

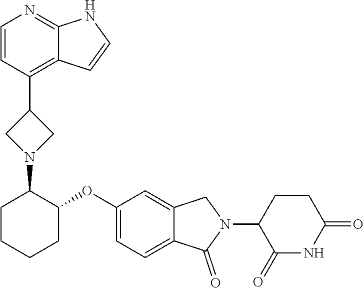 [SMILES: O=C1CCC(N2Cc3cc(O[C@@H]4CCCC[C@H]4N4CC(c5ccnc6[nH]ccc56)C4)ccc3C2=O)C(=O)N1]

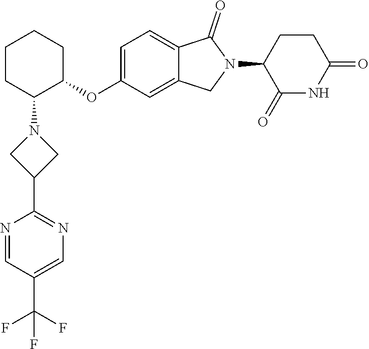 [SMILES: O=C1CC[C@H](N2Cc3cc(O[C@H]4CCCC[C@H]4N4CC(c5ncc(C(F)(F)F)cn5)C4)ccc3C2=O)C(=O)N1]

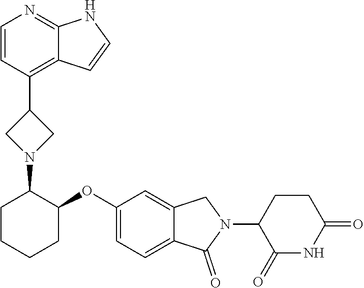 [SMILES: O=C1CCC(N2Cc3cc(O[C@H]4CCCC[C@H]4N4CC(c5ccnc6[nH]ccc56)C4)ccc3C2=O)C(=O)N1]

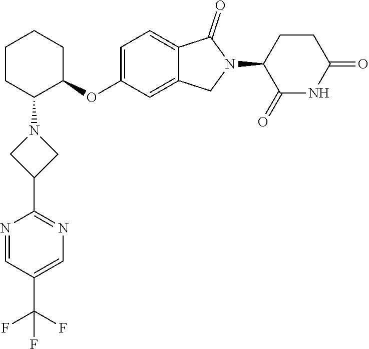 [SMILES: O=C1CC[C@H](N2Cc3cc(O[C@@H]4CCCC[C@H]4N4CC(c5ncc(C(F)(F)F)cn5)C4)ccc3C2=O)C(=O)N1]

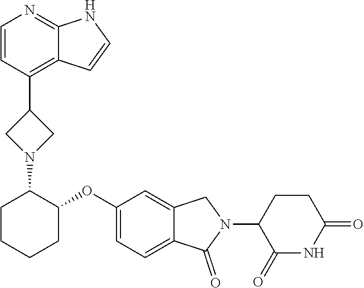 [SMILES: O=C1CCC(N2Cc3cc(O[C@@H]4CCCC[C@@H]4N4CC(c5ccnc6[nH]ccc56)C4)ccc3C2=O)C(=O)N1]

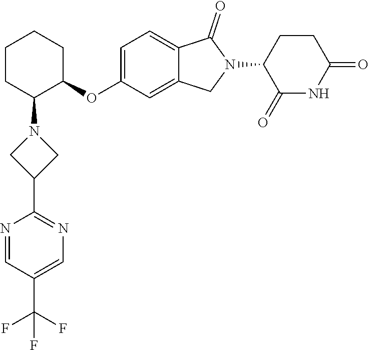 [SMILES: O=C1CC[C@@H](N2Cc3cc(O[C@@H]4CCCC[C@@H]4N4CC(c5ncc(C(F)(F)F)cn5)C4)ccc3C2=O)C(=O)N1]

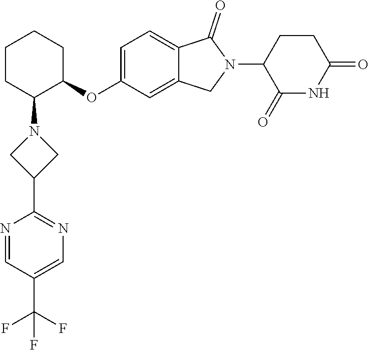 [SMILES: O=C1CCC(N2Cc3cc(O[C@@H]4CCCC[C@@H]4N4CC(c5ncc(C(F)(F)F)cn5)C4)ccc3C2=O)C(=O)N1]

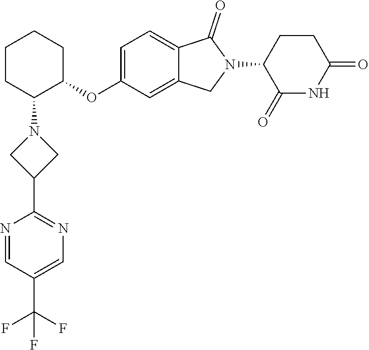 [SMILES: O=C1CC[C@@H](N2Cc3cc(O[C@H]4CCCC[C@H]4N4CC(c5ncc(C(F)(F)F)cn5)C4)ccc3C2=O)C(=O)N1]

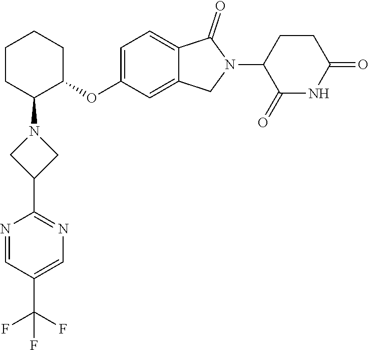 [SMILES: O=C1CCC(N2Cc3cc(O[C@H]4CCCC[C@@H]4N4CC(c5ncc(C(F)(F)F)cn5)C4)ccc3C2=O)C(=O)N1]

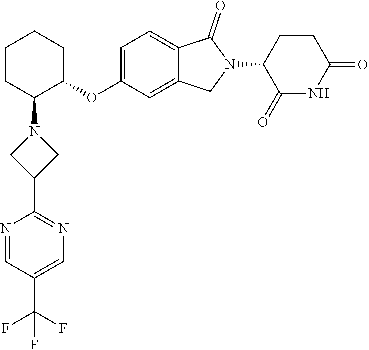 [SMILES: O=C1CC[C@@H](N2Cc3cc(O[C@H]4CCCC[C@@H]4N4CC(c5ncc(C(F)(F)F)cn5)C4)ccc3C2=O)C(=O)N1]

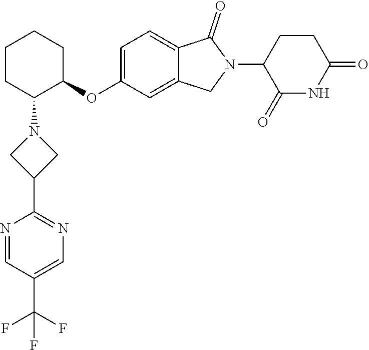 [SMILES: O=C1CCC(N2Cc3cc(O[C@@H]4CCCC[C@H]4N4CC(c5ncc(C(F)(F)F)cn5)C4)ccc3C2=O)C(=O)N1]

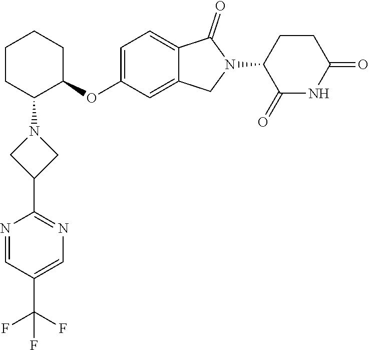 [SMILES: O=C1CC[C@@H](N2Cc3cc(O[C@@H]4CCCC[C@H]4N4CC(c5ncc(C(F)(F)F)cn5)C4)ccc3C2=O)C(=O)N1]

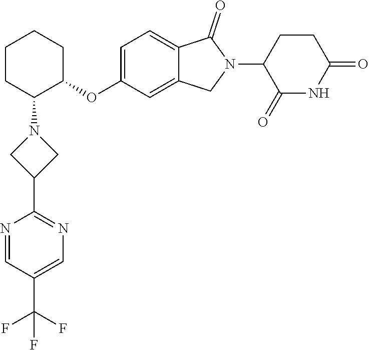 [SMILES: O=C1CCC(N2Cc3cc(O[C@H]4CCCC[C@H]4N4CC(c5ncc(C(F)(F)F)cn5)C4)ccc3C2=O)C(=O)N1]

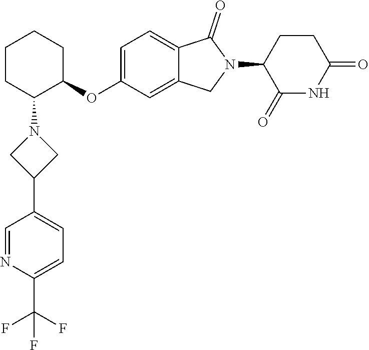 [SMILES: O=C1CC[C@H](N2Cc3cc(O[C@@H]4CCCC[C@H]4N4CC(c5ccc(C(F)(F)F)nc5)C4)ccc3C2=O)C(=O)N1]

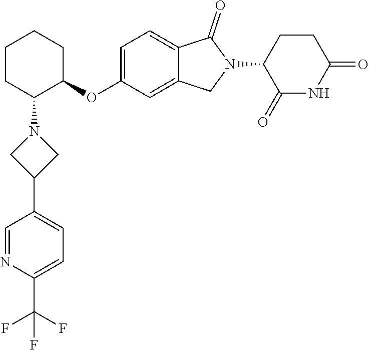 [SMILES: O=C1CC[C@@H](N2Cc3cc(O[C@@H]4CCCC[C@H]4N4CC(c5ccc(C(F)(F)F)nc5)C4)ccc3C2=O)C(=O)N1]

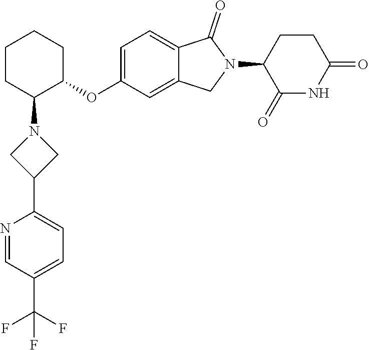 [SMILES: O=C1CC[C@H](N2Cc3cc(O[C@H]4CCCC[C@@H]4N4CC(c5ccc(C(F)(F)F)cn5)C4)ccc3C2=O)C(=O)N1]

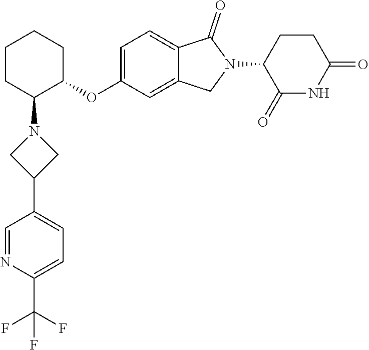 [SMILES: O=C1CC[C@@H](N2Cc3cc(O[C@H]4CCCC[C@@H]4N4CC(c5ccc(C(F)(F)F)nc5)C4)ccc3C2=O)C(=O)N1]

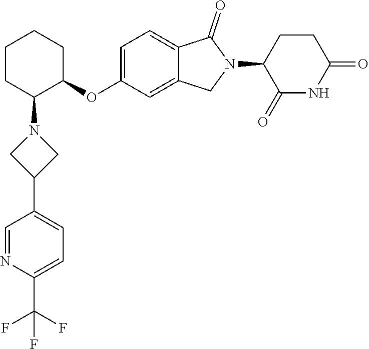 [SMILES: O=C1CC[C@H](N2Cc3cc(O[C@@H]4CCCC[C@@H]4N4CC(c5ccc(C(F)(F)F)nc5)C4)ccc3C2=O)C(=O)N1]

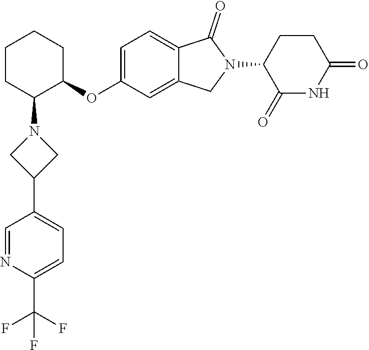 [SMILES: O=C1CC[C@@H](N2Cc3cc(O[C@@H]4CCCC[C@@H]4N4CC(c5ccc(C(F)(F)F)nc5)C4)ccc3C2=O)C(=O)N1]

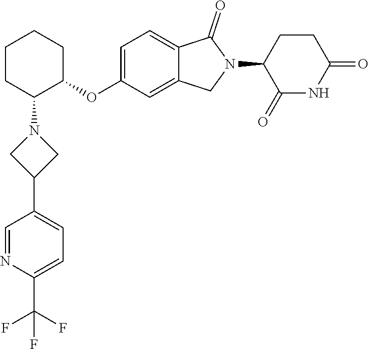 [SMILES: O=C1CC[C@H](N2Cc3cc(O[C@H]4CCCC[C@H]4N4CC(c5ccc(C(F)(F)F)nc5)C4)ccc3C2=O)C(=O)N1]

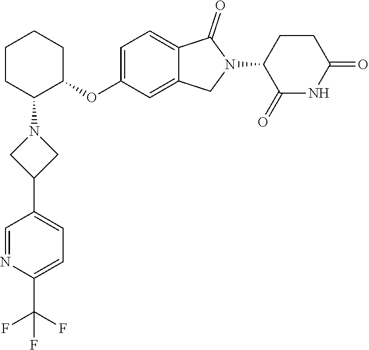 [SMILES: O=C1CC[C@@H](N2Cc3cc(O[C@H]4CCCC[C@H]4N4CC(c5ccc(C(F)(F)F)nc5)C4)ccc3C2=O)C(=O)N1]

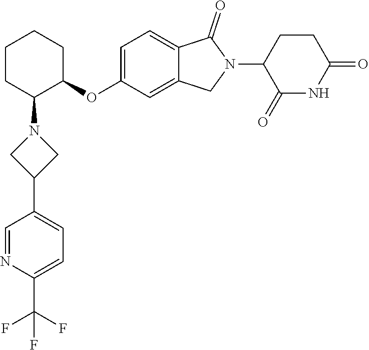 [SMILES: O=C1CCC(N2Cc3cc(O[C@@H]4CCCC[C@@H]4N4CC(c5ccc(C(F)(F)F)nc5)C4)ccc3C2=O)C(=O)N1]

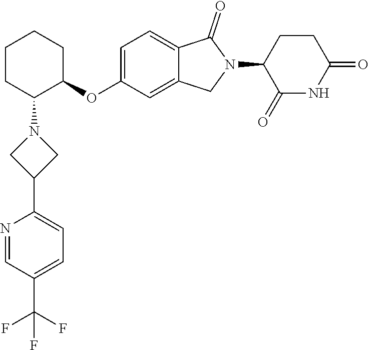 [SMILES: O=C1CC[C@H](N2Cc3cc(O[C@@H]4CCCC[C@H]4N4CC(c5ccc(C(F)(F)F)cn5)C4)ccc3C2=O)C(=O)N1]

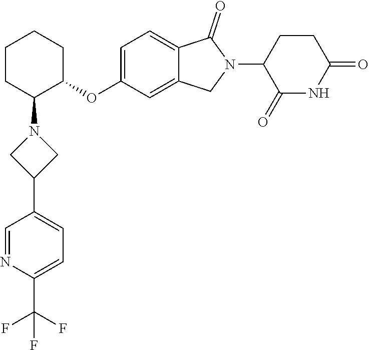 [SMILES: O=C1CCC(N2Cc3cc(O[C@H]4CCCC[C@@H]4N4CC(c5ccc(C(F)(F)F)nc5)C4)ccc3C2=O)C(=O)N1]

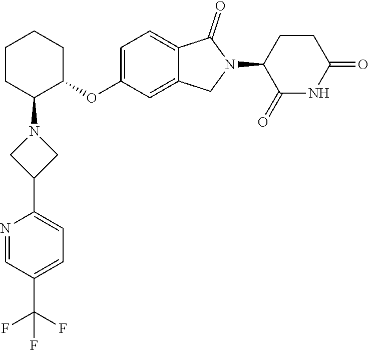 [SMILES: O=C1CC[C@H](N2Cc3cc(O[C@H]4CCCC[C@@H]4N4CC(c5ccc(C(F)(F)F)cn5)C4)ccc3C2=O)C(=O)N1]

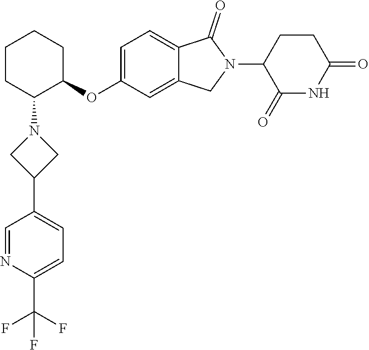 [SMILES: O=C1CCC(N2Cc3cc(O[C@@H]4CCCC[C@H]4N4CC(c5ccc(C(F)(F)F)nc5)C4)ccc3C2=O)C(=O)N1]

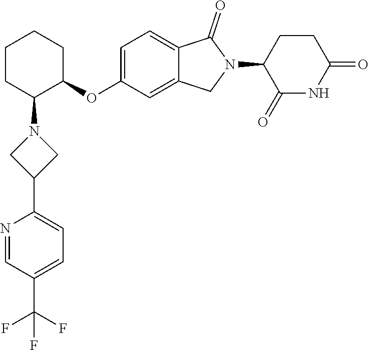 [SMILES: O=C1CC[C@H](N2Cc3cc(O[C@@H]4CCCC[C@@H]4N4CC(c5ccc(C(F)(F)F)cn5)C4)ccc3C2=O)C(=O)N1]

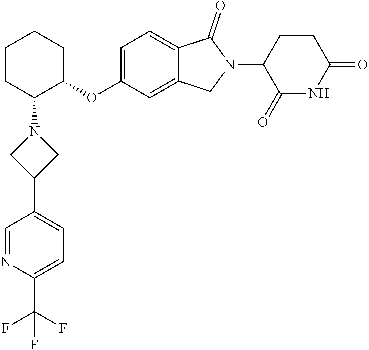 [SMILES: O=C1CCC(N2Cc3cc(O[C@H]4CCCC[C@H]4N4CC(c5ccc(C(F)(F)F)nc5)C4)ccc3C2=O)C(=O)N1]

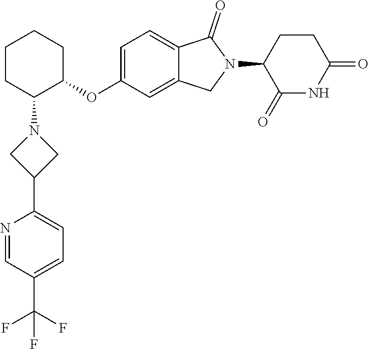 [SMILES: O=C1CC[C@H](N2Cc3cc(O[C@H]4CCCC[C@H]4N4CC(c5ccc(C(F)(F)F)cn5)C4)ccc3C2=O)C(=O)N1]

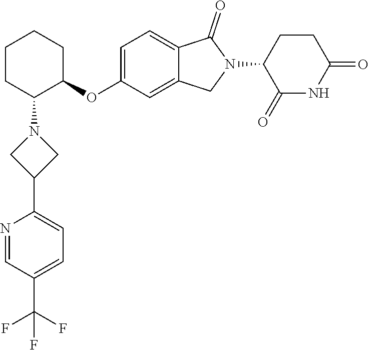 [SMILES: O=C1CC[C@@H](N2Cc3cc(O[C@@H]4CCCC[C@H]4N4CC(c5ccc(C(F)(F)F)cn5)C4)ccc3C2=O)C(=O)N1]

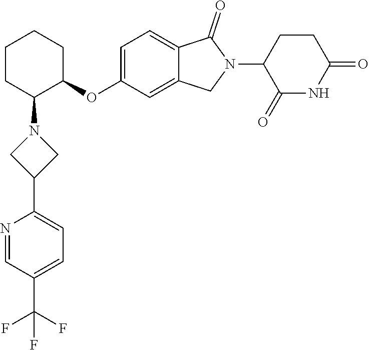 [SMILES: O=C1CCC(N2Cc3cc(O[C@@H]4CCCC[C@@H]4N4CC(c5ccc(C(F)(F)F)cn5)C4)ccc3C2=O)C(=O)N1]

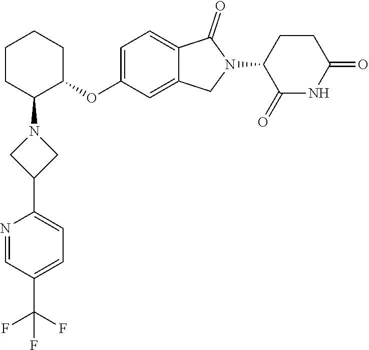 [SMILES: O=C1CC[C@@H](N2Cc3cc(O[C@H]4CCCC[C@@H]4N4CC(c5ccc(C(F)(F)F)cn5)C4)ccc3C2=O)C(=O)N1]

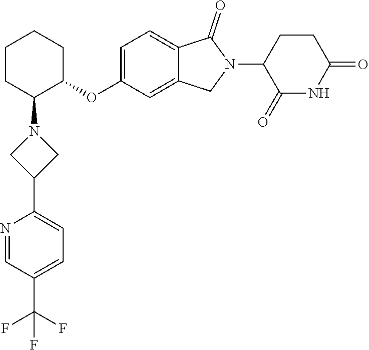 [SMILES: O=C1CCC(N2Cc3cc(O[C@H]4CCCC[C@@H]4N4CC(c5ccc(C(F)(F)F)cn5)C4)ccc3C2=O)C(=O)N1]

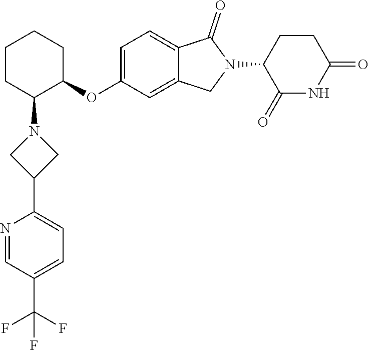 [SMILES: O=C1CC[C@@H](N2Cc3cc(O[C@@H]4CCCC[C@@H]4N4CC(c5ccc(C(F)(F)F)cn5)C4)ccc3C2=O)C(=O)N1]

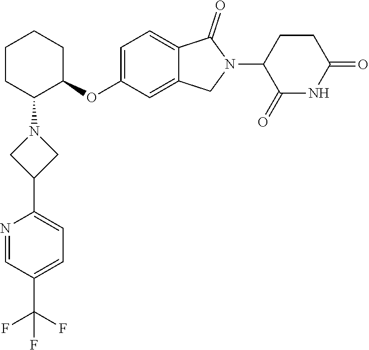 [SMILES: O=C1CCC(N2Cc3cc(O[C@@H]4CCCC[C@H]4N4CC(c5ccc(C(F)(F)F)cn5)C4)ccc3C2=O)C(=O)N1]

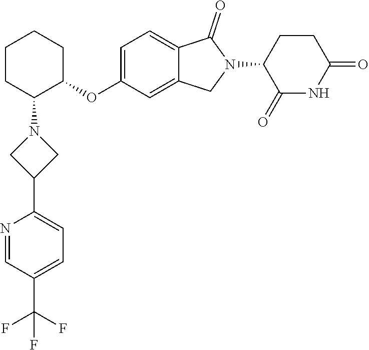 [SMILES: O=C1CC[C@@H](N2Cc3cc(O[C@H]4CCCC[C@H]4N4CC(c5ccc(C(F)(F)F)cn5)C4)ccc3C2=O)C(=O)N1]

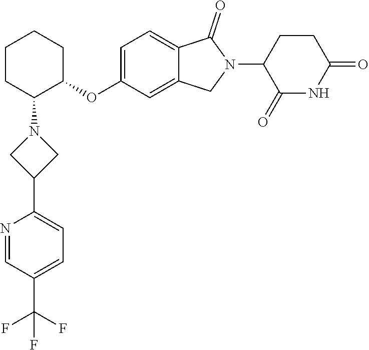 [SMILES: O=C1CCC(N2Cc3cc(O[C@H]4CCCC[C@H]4N4CC(c5ccc(C(F)(F)F)cn5)C4)ccc3C2=O)C(=O)N1]

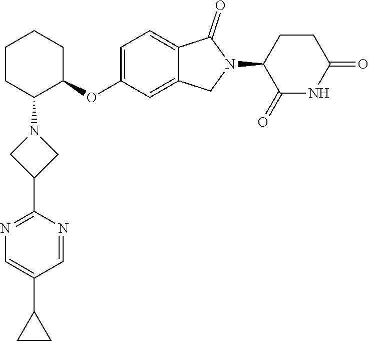 [SMILES: O=C1CC[C@H](N2Cc3cc(O[C@@H]4CCCC[C@H]4N4CC(c5ncc(C6CC6)cn5)C4)ccc3C2=O)C(=O)N1]

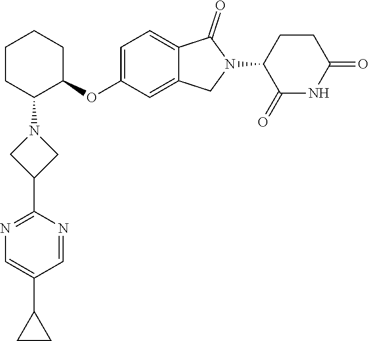 [SMILES: O=C1CC[C@@H](N2Cc3cc(O[C@@H]4CCCC[C@H]4N4CC(c5ncc(C6CC6)cn5)C4)ccc3C2=O)C(=O)N1]

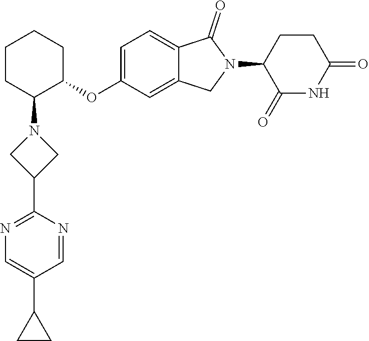 [SMILES: O=C1CC[C@H](N2Cc3cc(O[C@H]4CCCC[C@@H]4N4CC(c5ncc(C6CC6)cn5)C4)ccc3C2=O)C(=O)N1]

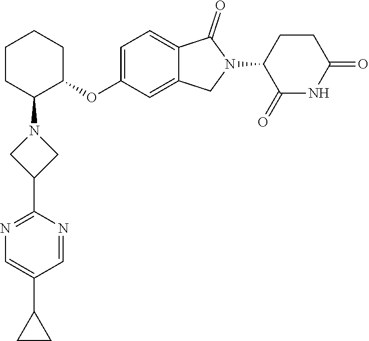 [SMILES: O=C1CC[C@@H](N2Cc3cc(O[C@H]4CCCC[C@@H]4N4CC(c5ncc(C6CC6)cn5)C4)ccc3C2=O)C(=O)N1]

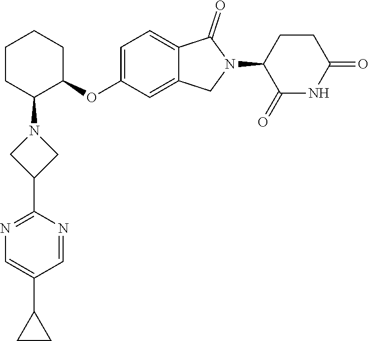 [SMILES: O=C1CC[C@H](N2Cc3cc(O[C@@H]4CCCC[C@@H]4N4CC(c5ncc(C6CC6)cn5)C4)ccc3C2=O)C(=O)N1]

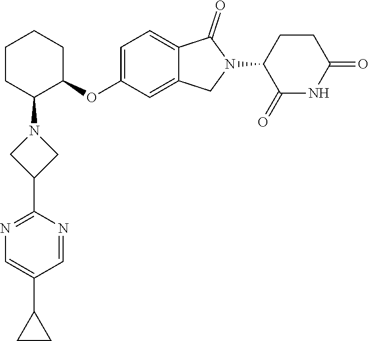 [SMILES: O=C1CC[C@@H](N2Cc3cc(O[C@@H]4CCCC[C@@H]4N4CC(c5ncc(C6CC6)cn5)C4)ccc3C2=O)C(=O)N1]

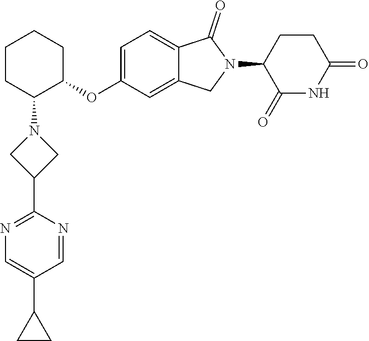 [SMILES: O=C1CC[C@H](N2Cc3cc(O[C@H]4CCCC[C@H]4N4CC(c5ncc(C6CC6)cn5)C4)ccc3C2=O)C(=O)N1]

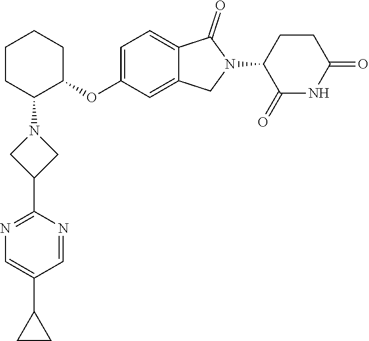 [SMILES: O=C1CC[C@@H](N2Cc3cc(O[C@H]4CCCC[C@H]4N4CC(c5ncc(C6CC6)cn5)C4)ccc3C2=O)C(=O)N1]

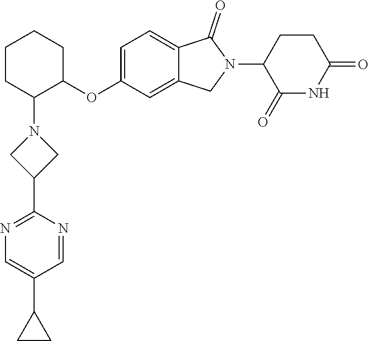 [SMILES: O=C1CCC(N2Cc3cc(OC4CCCCC4N4CC(c5ncc(C6CC6)cn5)C4)ccc3C2=O)C(=O)N1]

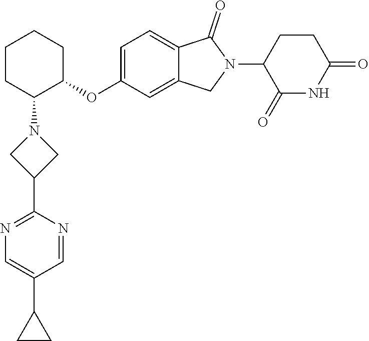 [SMILES: O=C1CCC(N2Cc3cc(O[C@H]4CCCC[C@H]4N4CC(c5ncc(C6CC6)cn5)C4)ccc3C2=O)C(=O)N1]

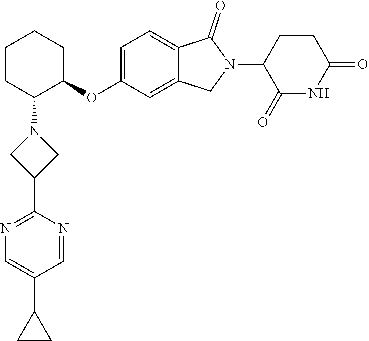 [SMILES: O=C1CCC(N2Cc3cc(O[C@@H]4CCCC[C@H]4N4CC(c5ncc(C6CC6)cn5)C4)ccc3C2=O)C(=O)N1]

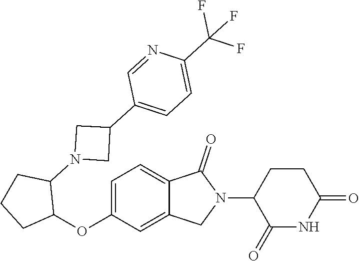 [SMILES: O=C1CCC(N2Cc3cc(OC4CCCC4N4CC(c5ccc(C(F)(F)F)nc5)C4)ccc3C2=O)C(=O)N1]

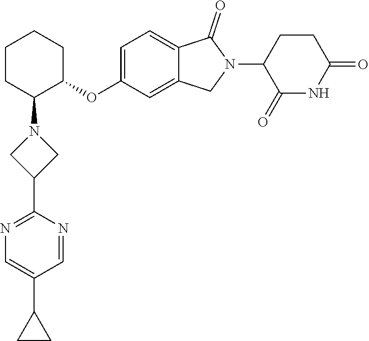 [SMILES: O=C1CCC(N2Cc3cc(O[C@H]4CCCC[C@@H]4N4CC(c5ncc(C6CC6)cn5)C4)ccc3C2=O)C(=O)N1]

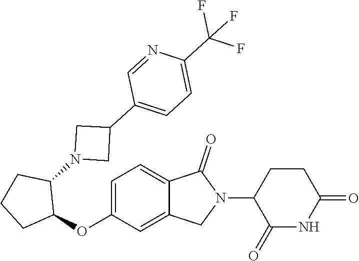 [SMILES: O=C1CCC(N2Cc3cc(O[C@H]4CCC[C@@H]4N4CC(c5ccc(C(F)(F)F)nc5)C4)ccc3C2=O)C(=O)N1]

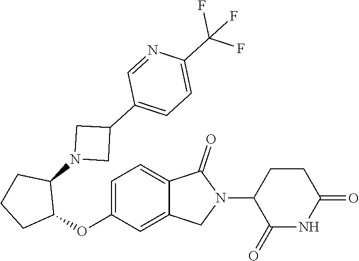 [SMILES: O=C1CCC(N2Cc3cc(O[C@@H]4CCC[C@H]4N4CC(c5ccc(C(F)(F)F)nc5)C4)ccc3C2=O)C(=O)N1]

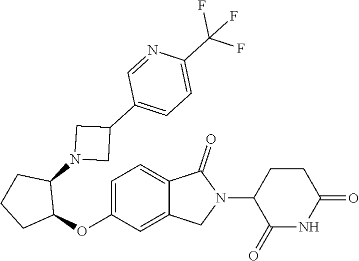 [SMILES: O=C1CCC(N2Cc3cc(O[C@H]4CCC[C@H]4N4CC(c5ccc(C(F)(F)F)nc5)C4)ccc3C2=O)C(=O)N1]

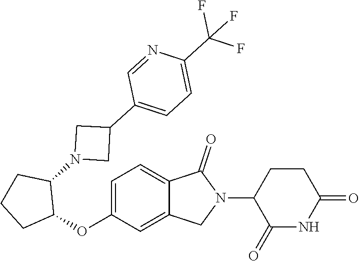 [SMILES: O=C1CCC(N2Cc3cc(O[C@@H]4CCC[C@@H]4N4CC(c5ccc(C(F)(F)F)nc5)C4)ccc3C2=O)C(=O)N1]

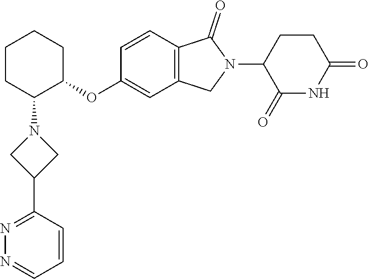 [SMILES: O=C1CCC(N2Cc3cc(O[C@H]4CCCC[C@H]4N4CC(c5cccnn5)C4)ccc3C2=O)C(=O)N1]

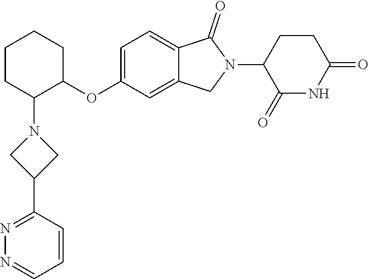 [SMILES: O=C1CCC(N2Cc3cc(OC4CCCCC4N4CC(c5cccnn5)C4)ccc3C2=O)C(=O)N1]

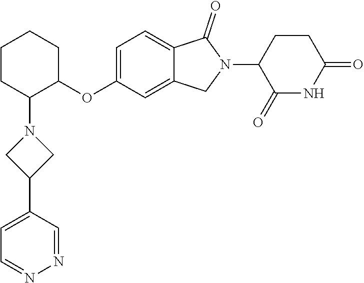 [SMILES: O=C1CCC(N2Cc3cc(OC4CCCCC4N4CC(c5ccnnc5)C4)ccc3C2=O)C(=O)N1]

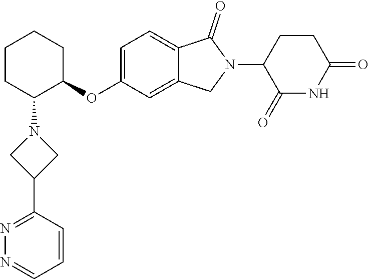 [SMILES: O=C1CCC(N2Cc3cc(O[C@@H]4CCCC[C@H]4N4CC(c5cccnn5)C4)ccc3C2=O)C(=O)N1]

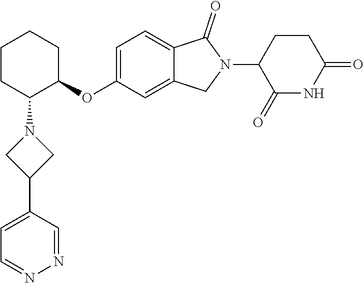 [SMILES: O=C1CCC(N2Cc3cc(O[C@@H]4CCCC[C@H]4N4CC(c5ccnnc5)C4)ccc3C2=O)C(=O)N1]

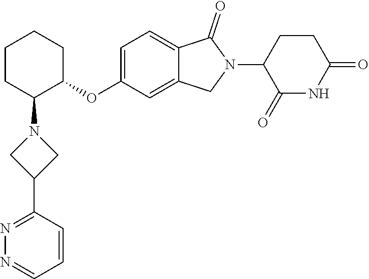 [SMILES: O=C1CCC(N2Cc3cc(O[C@H]4CCCC[C@@H]4N4CC(c5cccnn5)C4)ccc3C2=O)C(=O)N1]

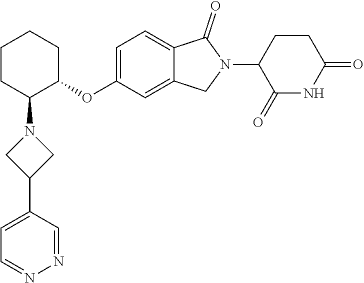 [SMILES: O=C1CCC(N2Cc3cc(O[C@H]4CCCC[C@@H]4N4CC(c5ccnnc5)C4)ccc3C2=O)C(=O)N1]

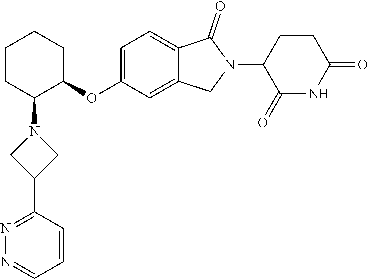 [SMILES: O=C1CCC(N2Cc3cc(O[C@@H]4CCCC[C@@H]4N4CC(c5cccnn5)C4)ccc3C2=O)C(=O)N1]

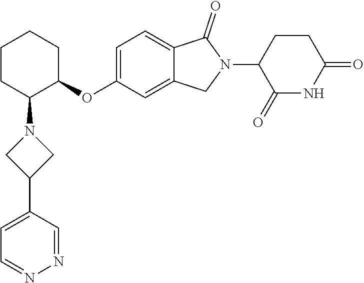 [SMILES: O=C1CCC(N2Cc3cc(O[C@@H]4CCCC[C@@H]4N4CC(c5ccnnc5)C4)ccc3C2=O)C(=O)N1]

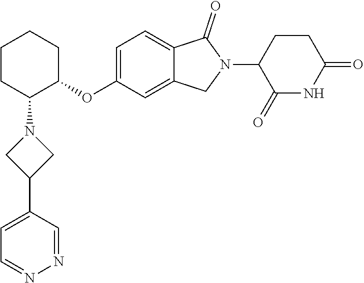 [SMILES: O=C1CCC(N2Cc3cc(O[C@H]4CCCC[C@H]4N4CC(c5ccnnc5)C4)ccc3C2=O)C(=O)N1]

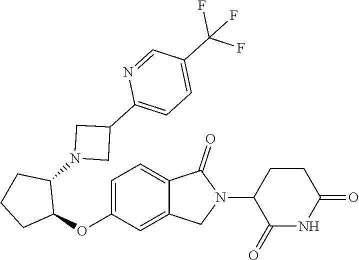 [SMILES: O=C1CCC(N2Cc3cc(O[C@H]4CCC[C@@H]4N4CC(c5ccc(C(F)(F)F)cn5)C4)ccc3C2=O)C(=O)N1]

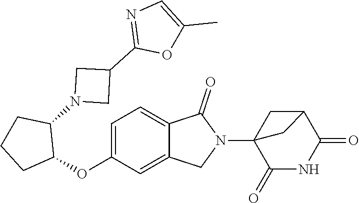 [SMILES: Cc1cnc(C2CN([C@H]3CCC[C@H]3Oc3ccc4c(c3)CN(C35CC(C3)C(=O)NC5=O)C4=O)C2)o1]

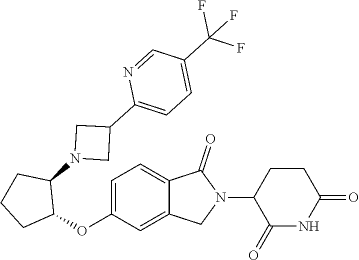 [SMILES: O=C1CCC(N2Cc3cc(O[C@@H]4CCC[C@H]4N4CC(c5ccc(C(F)(F)F)cn5)C4)ccc3C2=O)C(=O)N1]

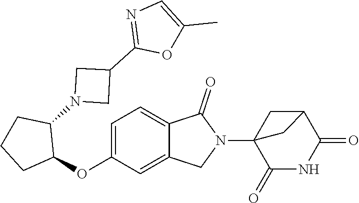 [SMILES: Cc1cnc(C2CN([C@H]3CCC[C@@H]3Oc3ccc4c(c3)CN(C35CC(C3)C(=O)NC5=O)C4=O)C2)o1]

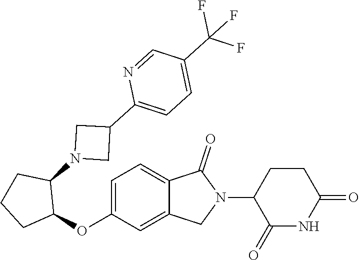 [SMILES: O=C1CCC(N2Cc3cc(O[C@H]4CCC[C@H]4N4CC(c5ccc(C(F)(F)F)cn5)C4)ccc3C2=O)C(=O)N1]

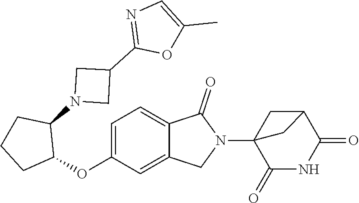 [SMILES: Cc1cnc(C2CN([C@@H]3CCC[C@H]3Oc3ccc4c(c3)CN(C35CC(C3)C(=O)NC5=O)C4=O)C2)o1]

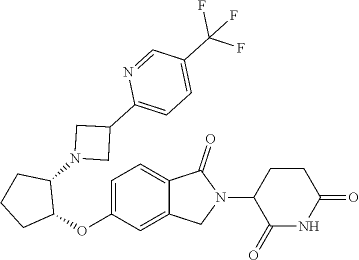 [SMILES: O=C1CCC(N2Cc3cc(O[C@@H]4CCC[C@@H]4N4CC(c5ccc(C(F)(F)F)cn5)C4)ccc3C2=O)C(=O)N1]

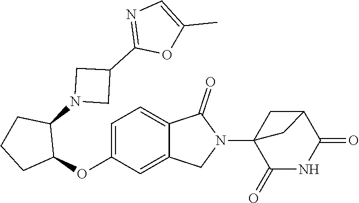 [SMILES: Cc1cnc(C2CN([C@@H]3CCC[C@@H]3Oc3ccc4c(c3)CN(C35CC(C3)C(=O)NC5=O)C4=O)C2)o1]

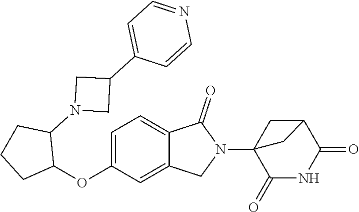 [SMILES: O=C1NC(=O)C2(N3Cc4cc(OC5CCCC5N5CC(c6ccncc6)C5)ccc4C3=O)CC1C2]

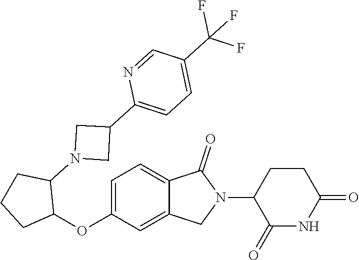 [SMILES: O=C1CCC(N2Cc3cc(OC4CCCC4N4CC(c5ccc(C(F)(F)F)cn5)C4)ccc3C2=O)C(=O)N1]

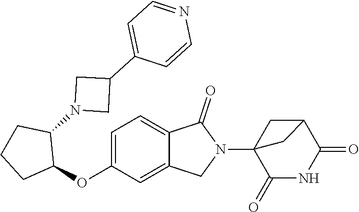 [SMILES: O=C1NC(=O)C2(N3Cc4cc(O[C@H]5CCC[C@@H]5N5CC(c6ccncc6)C5)ccc4C3=O)CC1C2]

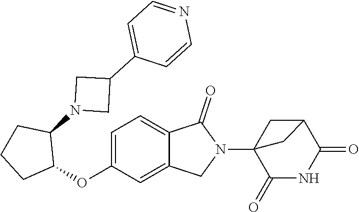 [SMILES: O=C1NC(=O)C2(N3Cc4cc(O[C@@H]5CCC[C@H]5N5CC(c6ccncc6)C5)ccc4C3=O)CC1C2]

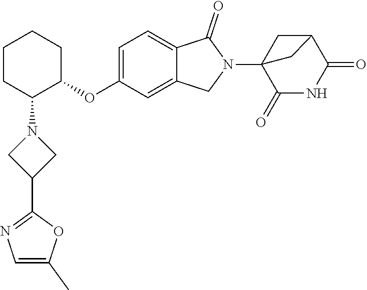 [SMILES: Cc1cnc(C2CN([C@@H]3CCCC[C@@H]3Oc3ccc4c(c3)CN(C35CC(C3)C(=O)NC5=O)C4=O)C2)o1]

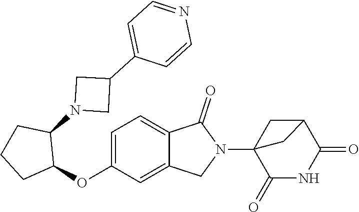 [SMILES: O=C1NC(=O)C2(N3Cc4cc(O[C@H]5CCC[C@H]5N5CC(c6ccncc6)C5)ccc4C3=O)CC1C2]

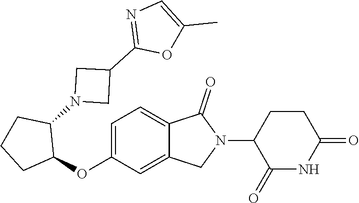 [SMILES: Cc1cnc(C2CN([C@H]3CCC[C@@H]3Oc3ccc4c(c3)CN(C3CCC(=O)NC3=O)C4=O)C2)o1]

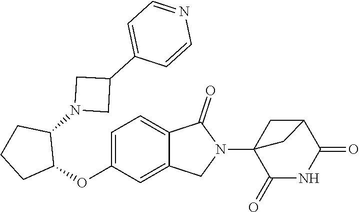 [SMILES: O=C1NC(=O)C2(N3Cc4cc(O[C@@H]5CCC[C@@H]5N5CC(c6ccncc6)C5)ccc4C3=O)CC1C2]

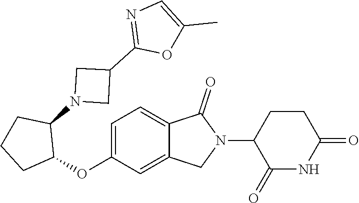 [SMILES: Cc1cnc(C2CN([C@@H]3CCC[C@H]3Oc3ccc4c(c3)CN(C3CCC(=O)NC3=O)C4=O)C2)o1]

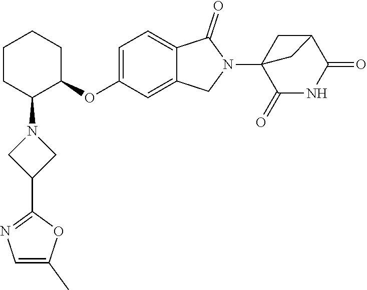 [SMILES: Cc1cnc(C2CN([C@H]3CCCC[C@H]3Oc3ccc4c(c3)CN(C35CC(C3)C(=O)NC5=O)C4=O)C2)o1]

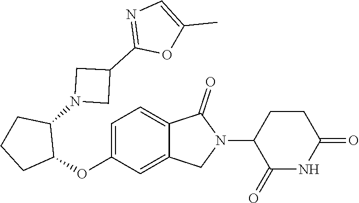 [SMILES: Cc1cnc(C2CN([C@H]3CCC[C@H]3Oc3ccc4c(c3)CN(C3CCC(=O)NC3=O)C4=O)C2)o1]

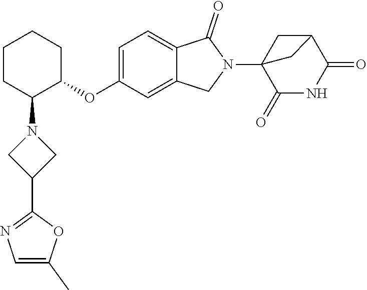 [SMILES: Cc1cnc(C2CN([C@H]3CCCC[C@@H]3Oc3ccc4c(c3)CN(C35CC(C3)C(=O)NC5=O)C4=O)C2)o1]

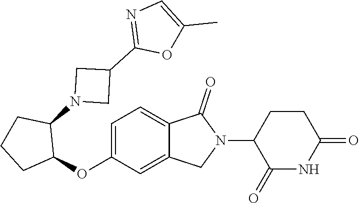 [SMILES: Cc1cnc(C2CN([C@@H]3CCC[C@@H]3Oc3ccc4c(c3)CN(C3CCC(=O)NC3=O)C4=O)C2)o1]

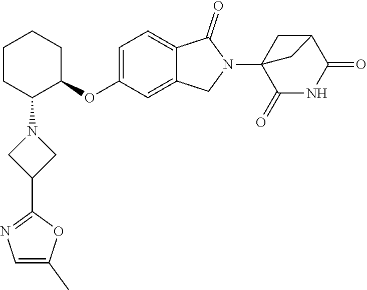 [SMILES: Cc1cnc(C2CN([C@@H]3CCCC[C@H]3Oc3ccc4c(c3)CN(C35CC(C3)C(=O)NC5=O)C4=O)C2)o1]

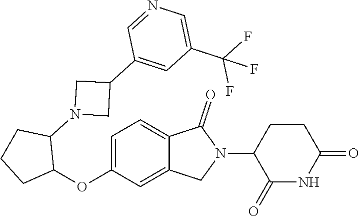 [SMILES: O=C1CCC(N2Cc3cc(OC4CCCC4N4CC(c5cncc(C(F)(F)F)c5)C4)ccc3C2=O)C(=O)N1]

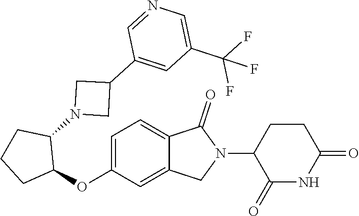 [SMILES: O=C1CCC(N2Cc3cc(O[C@H]4CCC[C@@H]4N4CC(c5cncc(C(F)(F)F)c5)C4)ccc3C2=O)C(=O)N1]

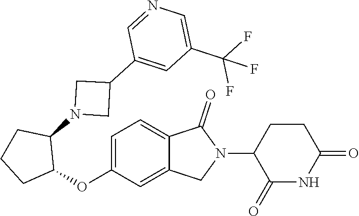 [SMILES: O=C1CCC(N2Cc3cc(O[C@@H]4CCC[C@H]4N4CC(c5cncc(C(F)(F)F)c5)C4)ccc3C2=O)C(=O)N1]

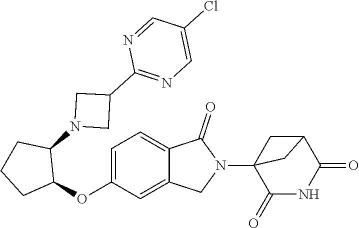 [SMILES: O=C1NC(=O)C2(N3Cc4cc(O[C@H]5CCC[C@H]5N5CC(c6ncc(Cl)cn6)C5)ccc4C3=O)CC1C2]

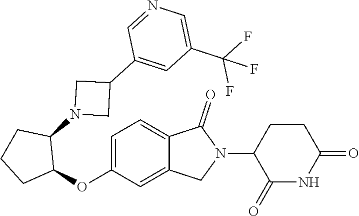 [SMILES: O=C1CCC(N2Cc3cc(O[C@H]4CCC[C@H]4N4CC(c5cncc(C(F)(F)F)c5)C4)ccc3C2=O)C(=O)N1]

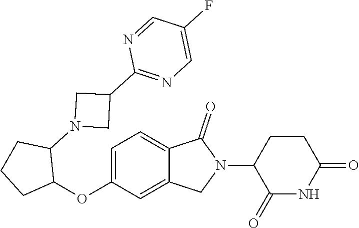 [SMILES: O=C1CCC(N2Cc3cc(OC4CCCC4N4CC(c5ncc(F)cn5)C4)ccc3C2=O)C(=O)N1]

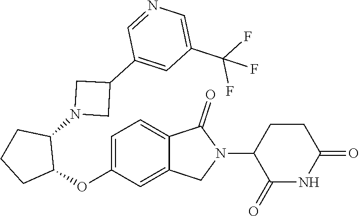 [SMILES: O=C1CCC(N2Cc3cc(O[C@@H]4CCC[C@@H]4N4CC(c5cncc(C(F)(F)F)c5)C4)ccc3C2=O)C(=O)N1]

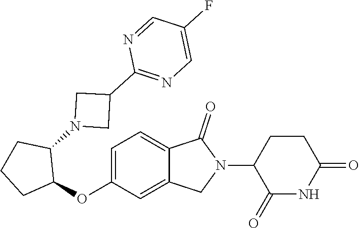 [SMILES: O=C1CCC(N2Cc3cc(O[C@H]4CCC[C@@H]4N4CC(c5ncc(F)cn5)C4)ccc3C2=O)C(=O)N1]

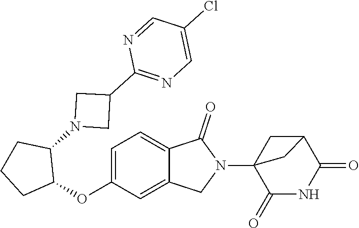 [SMILES: O=C1NC(=O)C2(N3Cc4cc(O[C@@H]5CCC[C@@H]5N5CC(c6ncc(Cl)cn6)C5)ccc4C3=O)CC1C2]

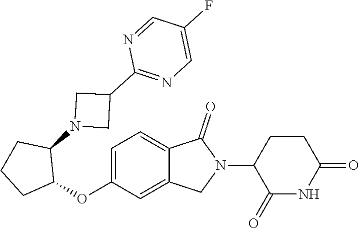 [SMILES: O=C1CCC(N2Cc3cc(O[C@@H]4CCC[C@H]4N4CC(c5ncc(F)cn5)C4)ccc3C2=O)C(=O)N1]

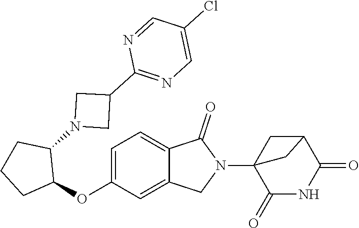 [SMILES: O=C1NC(=O)C2(N3Cc4cc(O[C@H]5CCC[C@@H]5N5CC(c6ncc(Cl)cn6)C5)ccc4C3=O)CC1C2]

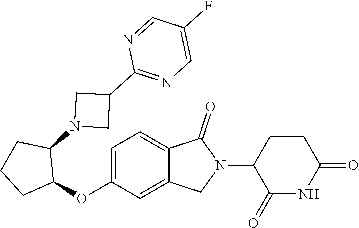 [SMILES: O=C1CCC(N2Cc3cc(O[C@H]4CCC[C@H]4N4CC(c5ncc(F)cn5)C4)ccc3C2=O)C(=O)N1]

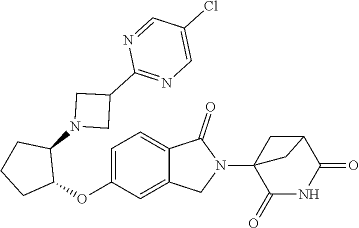 [SMILES: O=C1NC(=O)C2(N3Cc4cc(O[C@@H]5CCC[C@H]5N5CC(c6ncc(Cl)cn6)C5)ccc4C3=O)CC1C2]

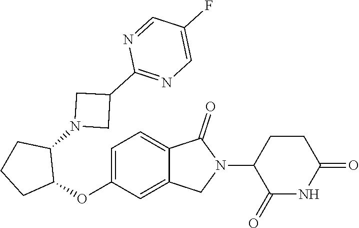 [SMILES: O=C1CCC(N2Cc3cc(O[C@@H]4CCC[C@@H]4N4CC(c5ncc(F)cn5)C4)ccc3C2=O)C(=O)N1]

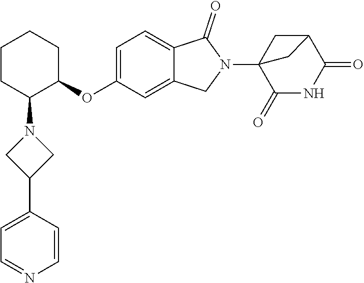 [SMILES: O=C1NC(=O)C2(N3Cc4cc(O[C@@H]5CCCC[C@@H]5N5CC(c6ccncc6)C5)ccc4C3=O)CC1C2]

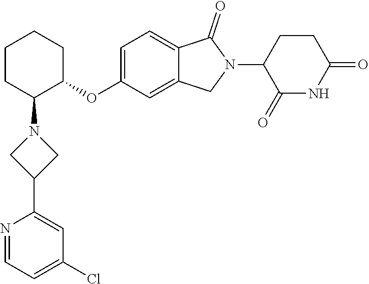 [SMILES: O=C1CCC(N2Cc3cc(O[C@H]4CCCC[C@@H]4N4CC(c5cc(Cl)ccn5)C4)ccc3C2=O)C(=O)N1]

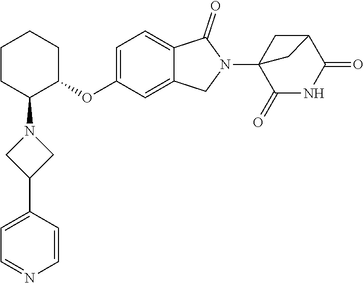 [SMILES: O=C1NC(=O)C2(N3Cc4cc(O[C@H]5CCCC[C@@H]5N5CC(c6ccncc6)C5)ccc4C3=O)CC1C2]

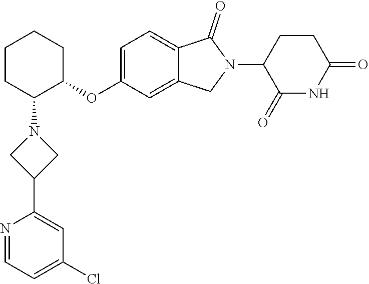 [SMILES: O=C1CCC(N2Cc3cc(O[C@H]4CCCC[C@H]4N4CC(c5cc(Cl)ccn5)C4)ccc3C2=O)C(=O)N1]

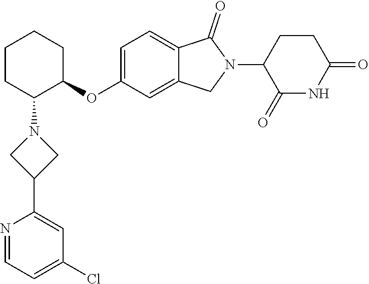 [SMILES: O=C1CCC(N2Cc3cc(O[C@@H]4CCCC[C@H]4N4CC(c5cc(Cl)ccn5)C4)ccc3C2=O)C(=O)N1]

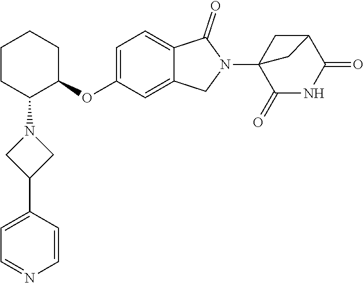 [SMILES: O=C1NC(=O)C2(N3Cc4cc(O[C@@H]5CCCC[C@H]5N5CC(c6ccncc6)C5)ccc4C3=O)CC1C2]

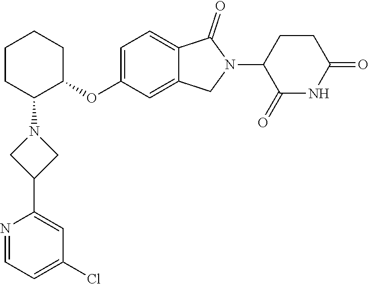 [SMILES: O=C1CCC(N2Cc3cc(O[C@H]4CCCC[C@H]4N4CC(c5cc(Cl)ccn5)C4)ccc3C2=O)C(=O)N1]

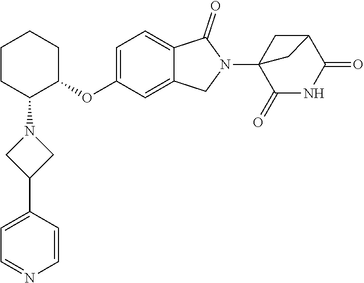 [SMILES: O=C1NC(=O)C2(N3Cc4cc(O[C@H]5CCCC[C@H]5N5CC(c6ccncc6)C5)ccc4C3=O)CC1C2]

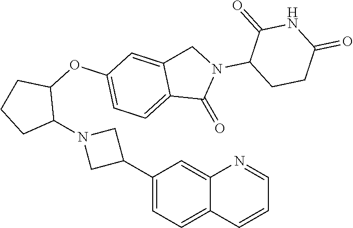 [SMILES: O=C1CCC(N2Cc3cc(OC4CCCC4N4CC(c5ccc6cccnc6c5)C4)ccc3C2=O)C(=O)N1]

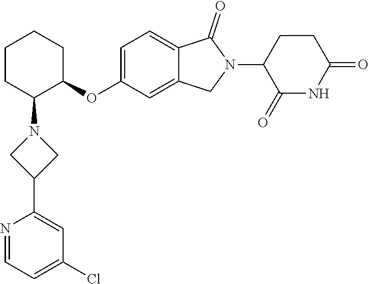 [SMILES: O=C1CCC(N2Cc3cc(O[C@@H]4CCCC[C@@H]4N4CC(c5cc(Cl)ccn5)C4)ccc3C2=O)C(=O)N1]

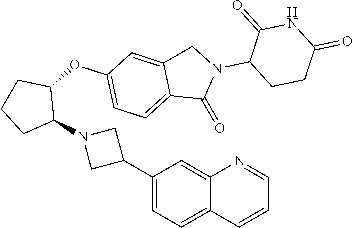 [SMILES: O=C1CCC(N2Cc3cc(O[C@H]4CCC[C@@H]4N4CC(c5ccc6cccnc6c5)C4)ccc3C2=O)C(=O)N1]

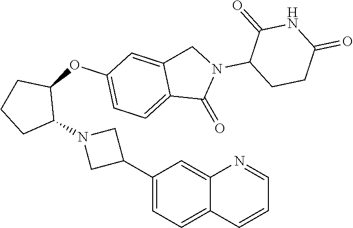 [SMILES: O=C1CCC(N2Cc3cc(O[C@@H]4CCC[C@H]4N4CC(c5ccc6cccnc6c5)C4)ccc3C2=O)C(=O)N1]

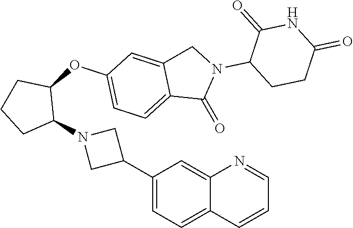 [SMILES: O=C1CCC(N2Cc3cc(O[C@@H]4CCC[C@@H]4N4CC(c5ccc6cccnc6c5)C4)ccc3C2=O)C(=O)N1]

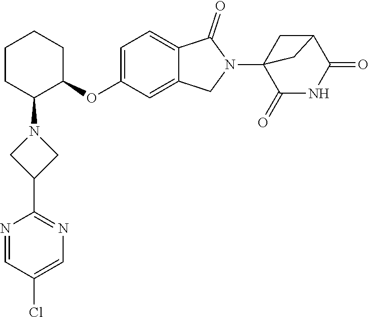 [SMILES: O=C1NC(=O)C2(N3Cc4cc(O[C@@H]5CCCC[C@@H]5N5CC(c6ncc(Cl)cn6)C5)ccc4C3=O)CC1C2]

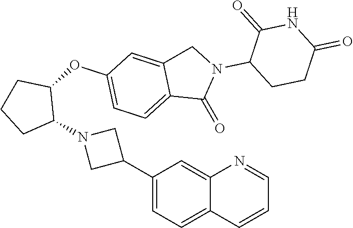 [SMILES: O=C1CCC(N2Cc3cc(O[C@H]4CCC[C@H]4N4CC(c5ccc6cccnc6c5)C4)ccc3C2=O)C(=O)N1]

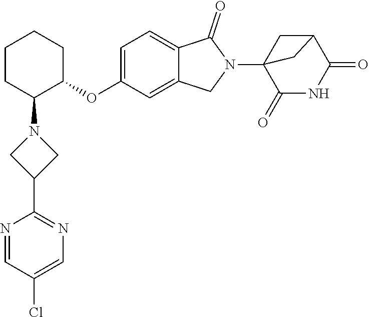 [SMILES: O=C1NC(=O)C2(N3Cc4cc(O[C@H]5CCCC[C@@H]5N5CC(c6ncc(Cl)cn6)C5)ccc4C3=O)CC1C2]

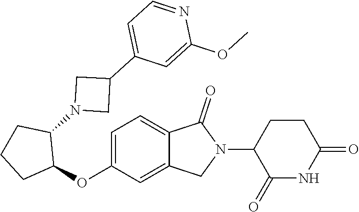 [SMILES: COc1cc(C2CN([C@H]3CCC[C@@H]3Oc3ccc4c(c3)CN(C3CCC(=O)NC3=O)C4=O)C2)ccn1]

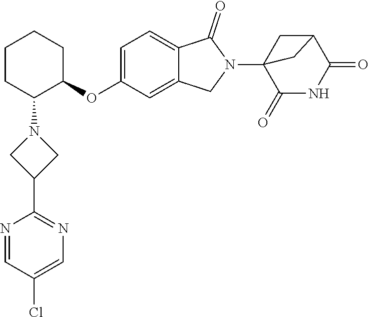 [SMILES: O=C1NC(=O)C2(N3Cc4cc(O[C@@H]5CCCC[C@H]5N5CC(c6ncc(Cl)cn6)C5)ccc4C3=O)CC1C2]

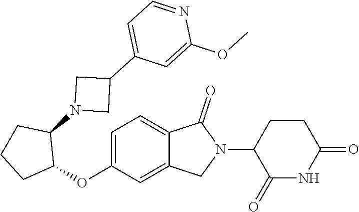 [SMILES: COc1cc(C2CN([C@@H]3CCC[C@H]3Oc3ccc4c(c3)CN(C3CCC(=O)NC3=O)C4=O)C2)ccn1]

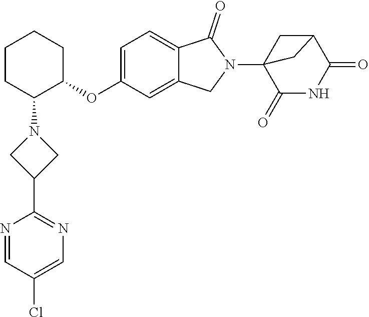 [SMILES: O=C1NC(=O)C2(N3Cc4cc(O[C@H]5CCCC[C@H]5N5CC(c6ncc(Cl)cn6)C5)ccc4C3=O)CC1C2]

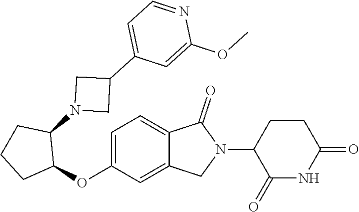 [SMILES: COc1cc(C2CN([C@@H]3CCC[C@@H]3Oc3ccc4c(c3)CN(C3CCC(=O)NC3=O)C4=O)C2)ccn1]

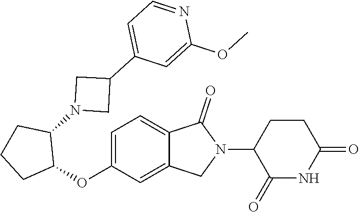 [SMILES: COc1cc(C2CN([C@H]3CCC[C@H]3Oc3ccc4c(c3)CN(C3CCC(=O)NC3=O)C4=O)C2)ccn1]

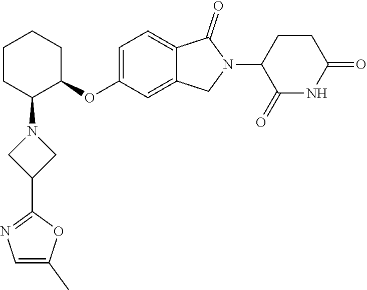 [SMILES: Cc1cnc(C2CN([C@H]3CCCC[C@H]3Oc3ccc4c(c3)CN(C3CCC(=O)NC3=O)C4=O)C2)o1]

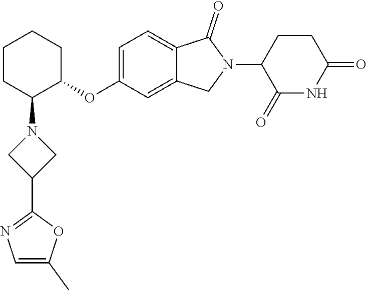 [SMILES: Cc1cnc(C2CN([C@H]3CCCC[C@@H]3Oc3ccc4c(c3)CN(C3CCC(=O)NC3=O)C4=O)C2)o1]

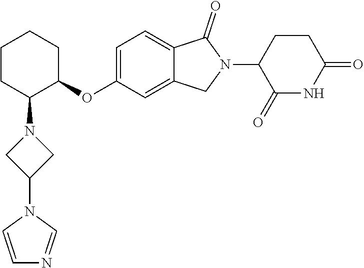 [SMILES: O=C1CCC(N2Cc3cc(O[C@@H]4CCCC[C@@H]4N4CC(n5ccnc5)C4)ccc3C2=O)C(=O)N1]

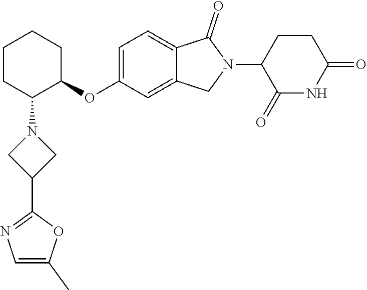 [SMILES: Cc1cnc(C2CN([C@@H]3CCCC[C@H]3Oc3ccc4c(c3)CN(C3CCC(=O)NC3=O)C4=O)C2)o1]

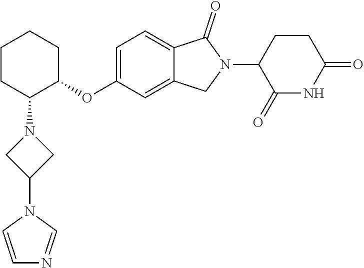 [SMILES: O=C1CCC(N2Cc3cc(O[C@H]4CCCC[C@H]4N4CC(n5ccnc5)C4)ccc3C2=O)C(=O)N1]

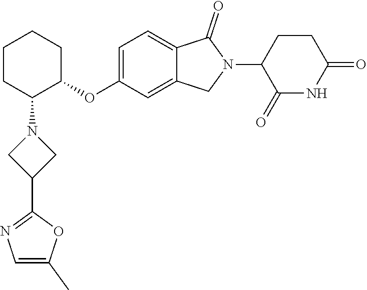 [SMILES: Cc1cnc(C2CN([C@@H]3CCCC[C@@H]3Oc3ccc4c(c3)CN(C3CCC(=O)NC3=O)C4=O)C2)o1]

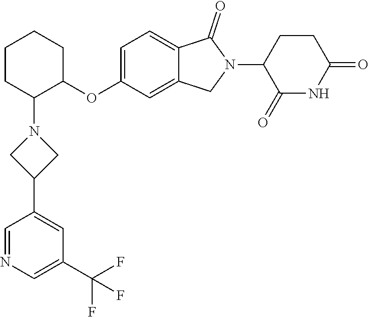 [SMILES: O=C1CCC(N2Cc3cc(OC4CCCCC4N4CC(c5cncc(C(F)(F)F)c5)C4)ccc3C2=O)C(=O)N1]

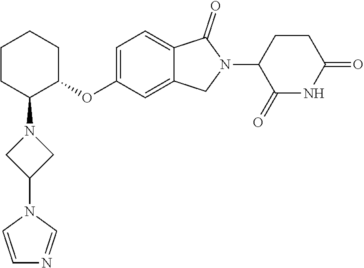 [SMILES: O=C1CCC(N2Cc3cc(O[C@H]4CCCC[C@@H]4N4CC(n5ccnc5)C4)ccc3C2=O)C(=O)N1]

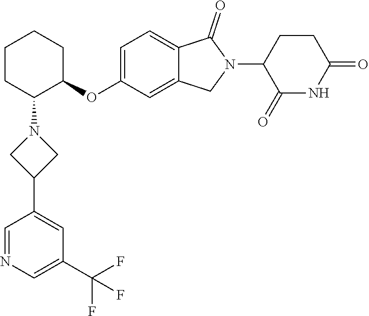 [SMILES: O=C1CCC(N2Cc3cc(O[C@@H]4CCCC[C@H]4N4CC(c5cncc(C(F)(F)F)c5)C4)ccc3C2=O)C(=O)N1]

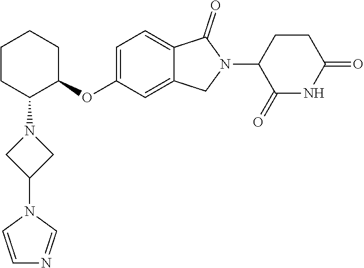 [SMILES: O=C1CCC(N2Cc3cc(O[C@@H]4CCCC[C@H]4N4CC(n5ccnc5)C4)ccc3C2=O)C(=O)N1]

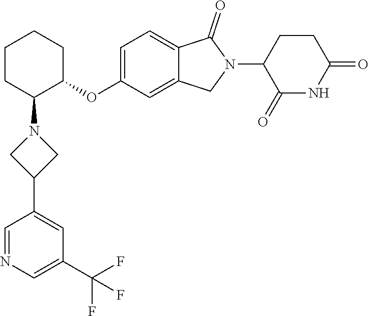 [SMILES: O=C1CCC(N2Cc3cc(O[C@H]4CCCC[C@@H]4N4CC(c5cncc(C(F)(F)F)c5)C4)ccc3C2=O)C(=O)N1]

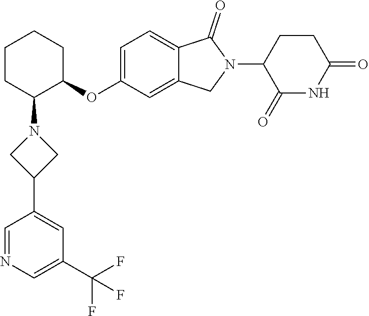 [SMILES: O=C1CCC(N2Cc3cc(O[C@@H]4CCCC[C@@H]4N4CC(c5cncc(C(F)(F)F)c5)C4)ccc3C2=O)C(=O)N1]

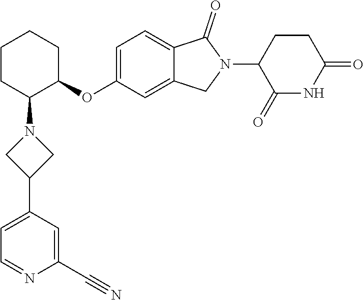 [SMILES: N#Cc1cc(C2CN([C@H]3CCCC[C@H]3Oc3ccc4c(c3)CN(C3CCC(=O)NC3=O)C4=O)C2)ccn1]

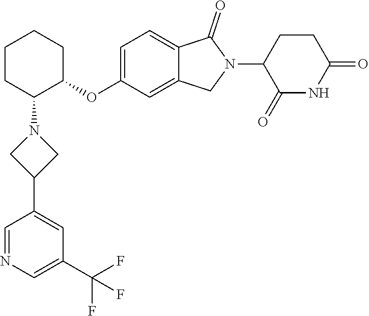 [SMILES: O=C1CCC(N2Cc3cc(O[C@H]4CCCC[C@H]4N4CC(c5cncc(C(F)(F)F)c5)C4)ccc3C2=O)C(=O)N1]

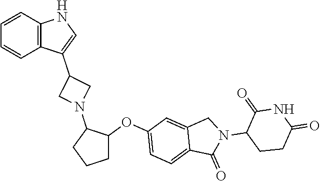 [SMILES: O=C1CCC(N2Cc3cc(OC4CCCC4N4CC(c5c[nH]c6ccccc56)C4)ccc3C2=O)C(=O)N1]

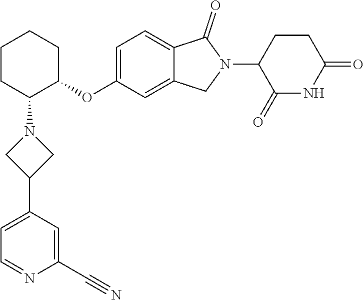 [SMILES: N#Cc1cc(C2CN([C@@H]3CCCC[C@@H]3Oc3ccc4c(c3)CN(C3CCC(=O)NC3=O)C4=O)C2)ccn1]

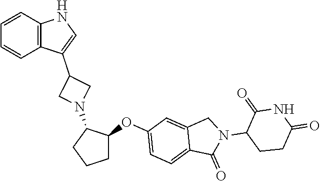 [SMILES: O=C1CCC(N2Cc3cc(O[C@H]4CCC[C@@H]4N4CC(c5c[nH]c6ccccc56)C4)ccc3C2=O)C(=O)N1]

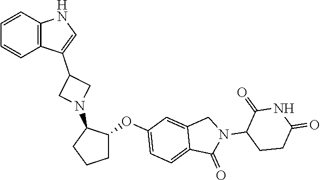 [SMILES: O=C1CCC(N2Cc3cc(O[C@@H]4CCC[C@H]4N4CC(c5c[nH]c6ccccc56)C4)ccc3C2=O)C(=O)N1]

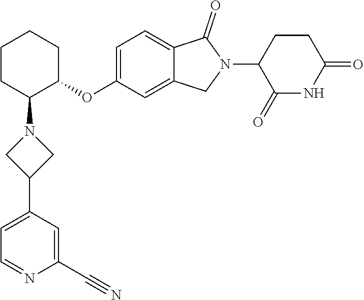 [SMILES: N#Cc1cc(C2CN([C@H]3CCCC[C@@H]3Oc3ccc4c(c3)CN(C3CCC(=O)NC3=O)C4=O)C2)ccn1]

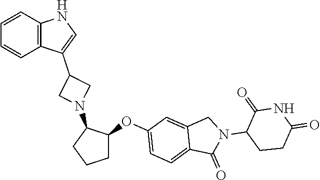 [SMILES: O=C1CCC(N2Cc3cc(O[C@H]4CCC[C@H]4N4CC(c5c[nH]c6ccccc56)C4)ccc3C2=O)C(=O)N1]

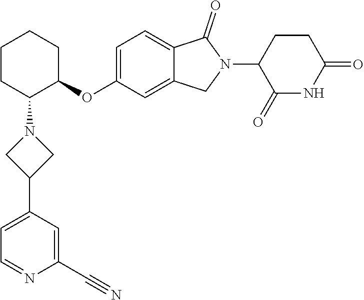 [SMILES: N#Cc1cc(C2CN([C@@H]3CCCC[C@H]3Oc3ccc4c(c3)CN(C3CCC(=O)NC3=O)C4=O)C2)ccn1]

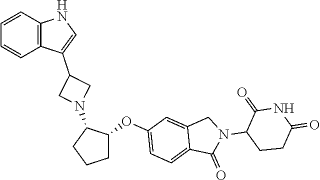 [SMILES: O=C1CCC(N2Cc3cc(O[C@@H]4CCC[C@@H]4N4CC(c5c[nH]c6ccccc56)C4)ccc3C2=O)C(=O)N1]

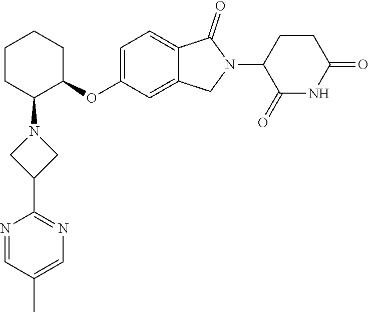 [SMILES: Cc1cnc(C2CN([C@H]3CCCC[C@H]3Oc3ccc4c(c3)CN(C3CCC(=O)NC3=O)C4=O)C2)nc1]

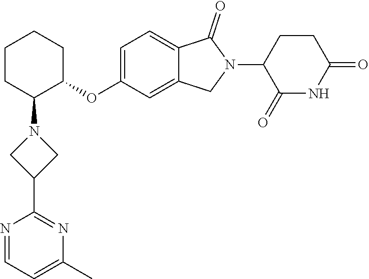 [SMILES: Cc1ccnc(C2CN([C@H]3CCCC[C@@H]3Oc3ccc4c(c3)CN(C3CCC(=O)NC3=O)C4=O)C2)n1]

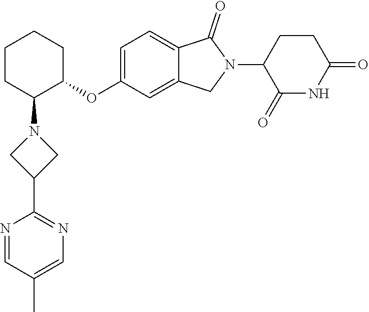 [SMILES: Cc1cnc(C2CN([C@H]3CCCC[C@@H]3Oc3ccc4c(c3)CN(C3CCC(=O)NC3=O)C4=O)C2)nc1]

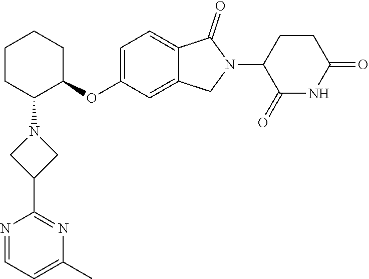 [SMILES: Cc1ccnc(C2CN([C@@H]3CCCC[C@H]3Oc3ccc4c(c3)CN(C3CCC(=O)NC3=O)C4=O)C2)n1]

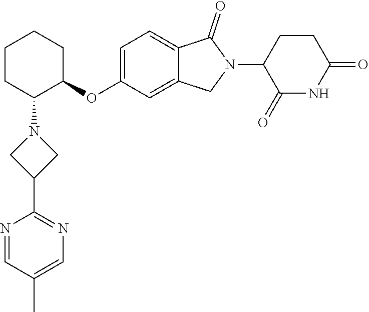 [SMILES: Cc1cnc(C2CN([C@@H]3CCCC[C@H]3Oc3ccc4c(c3)CN(C3CCC(=O)NC3=O)C4=O)C2)nc1]

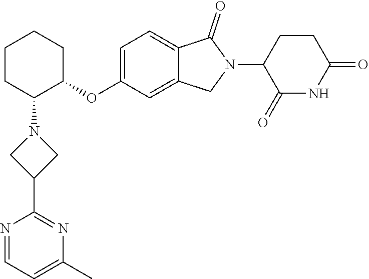 [SMILES: Cc1ccnc(C2CN([C@@H]3CCCC[C@@H]3Oc3ccc4c(c3)CN(C3CCC(=O)NC3=O)C4=O)C2)n1]

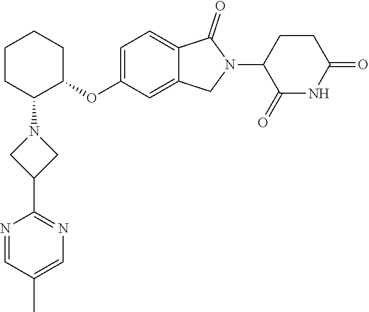 [SMILES: Cc1cnc(C2CN([C@@H]3CCCC[C@@H]3Oc3ccc4c(c3)CN(C3CCC(=O)NC3=O)C4=O)C2)nc1]

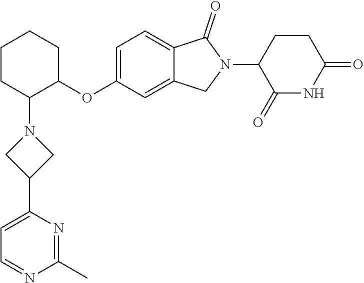 [SMILES: Cc1nccc(C2CN(C3CCCCC3Oc3ccc4c(c3)CN(C3CCC(=O)NC3=O)C4=O)C2)n1]

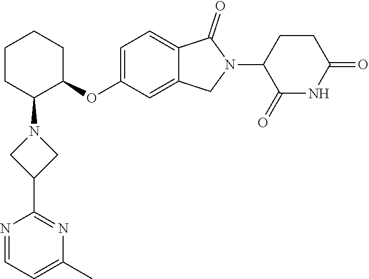 [SMILES: Cc1ccnc(C2CN([C@H]3CCCC[C@H]3Oc3ccc4c(c3)CN(C3CCC(=O)NC3=O)C4=O)C2)n1]

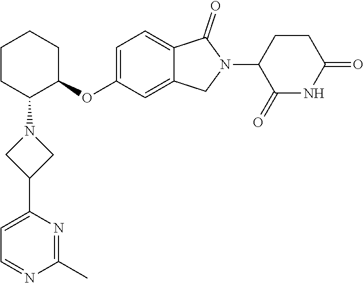 [SMILES: Cc1nccc(C2CN([C@@H]3CCCC[C@H]3Oc3ccc4c(c3)CN(C3CCC(=O)NC3=O)C4=O)C2)n1]

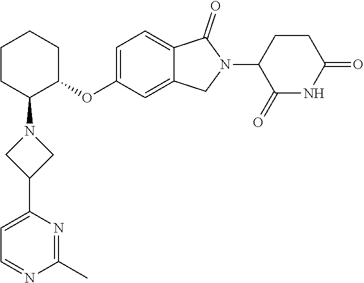 [SMILES: Cc1nccc(C2CN([C@H]3CCCC[C@@H]3Oc3ccc4c(c3)CN(C3CCC(=O)NC3=O)C4=O)C2)n1]

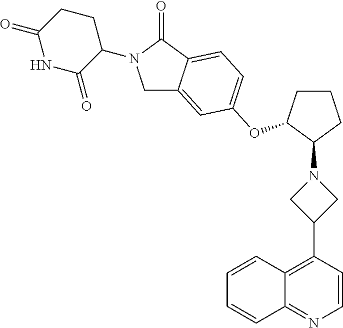 [SMILES: O=C1CCC(N2Cc3cc(O[C@@H]4CCC[C@H]4N4CC(c5ccnc6ccccc56)C4)ccc3C2=O)C(=O)N1]

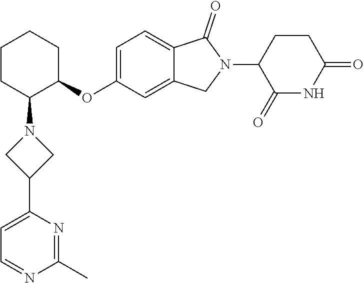 [SMILES: Cc1nccc(C2CN([C@H]3CCCC[C@H]3Oc3ccc4c(c3)CN(C3CCC(=O)NC3=O)C4=O)C2)n1]

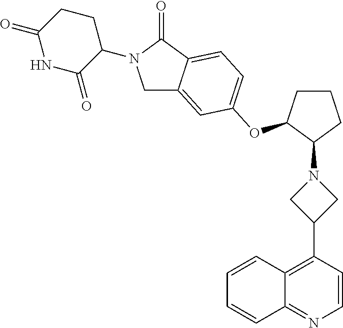 [SMILES: O=C1CCC(N2Cc3cc(O[C@H]4CCC[C@H]4N4CC(c5ccnc6ccccc56)C4)ccc3C2=O)C(=O)N1]

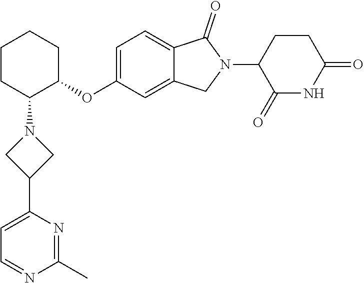 [SMILES: Cc1nccc(C2CN([C@@H]3CCCC[C@@H]3Oc3ccc4c(c3)CN(C3CCC(=O)NC3=O)C4=O)C2)n1]

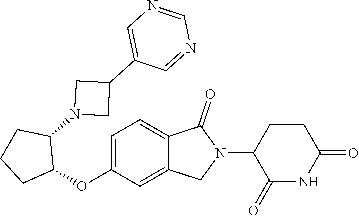 [SMILES: O=C1CCC(N2Cc3cc(O[C@@H]4CCC[C@@H]4N4CC(c5cncnc5)C4)ccc3C2=O)C(=O)N1]

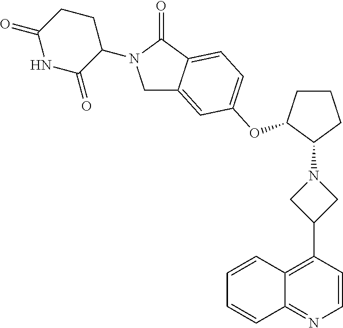 [SMILES: O=C1CCC(N2Cc3cc(O[C@@H]4CCC[C@@H]4N4CC(c5ccnc6ccccc56)C4)ccc3C2=O)C(=O)N1]

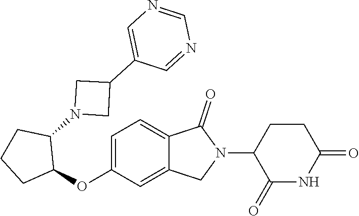 [SMILES: O=C1CCC(N2Cc3cc(O[C@H]4CCC[C@@H]4N4CC(c5cncnc5)C4)ccc3C2=O)C(=O)N1]

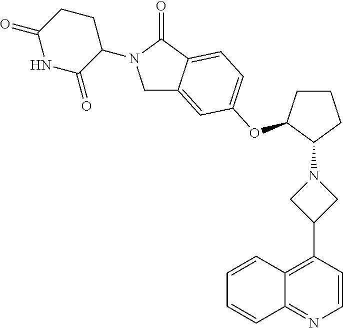 [SMILES: O=C1CCC(N2Cc3cc(O[C@H]4CCC[C@@H]4N4CC(c5ccnc6ccccc56)C4)ccc3C2=O)C(=O)N1]

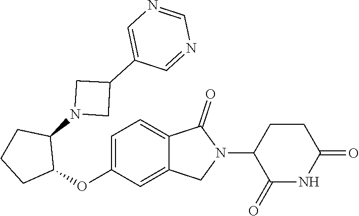 [SMILES: O=C1CCC(N2Cc3cc(O[C@@H]4CCC[C@H]4N4CC(c5cncnc5)C4)ccc3C2=O)C(=O)N1]

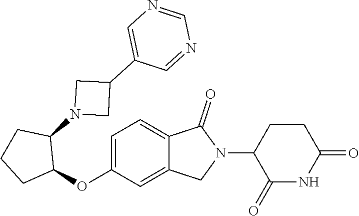 [SMILES: O=C1CCC(N2Cc3cc(O[C@H]4CCC[C@H]4N4CC(c5cncnc5)C4)ccc3C2=O)C(=O)N1]

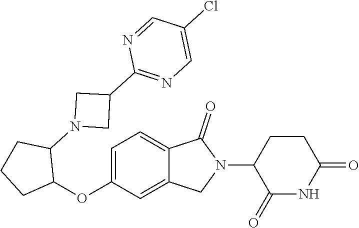 [SMILES: O=C1CCC(N2Cc3cc(OC4CCCC4N4CC(c5ncc(Cl)cn5)C4)ccc3C2=O)C(=O)N1]

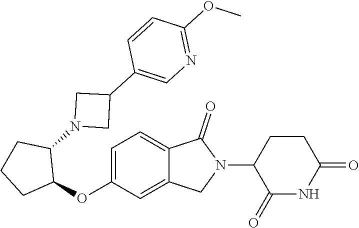 [SMILES: COc1ccc(C2CN([C@H]3CCC[C@@H]3Oc3ccc4c(c3)CN(C3CCC(=O)NC3=O)C4=O)C2)cn1]

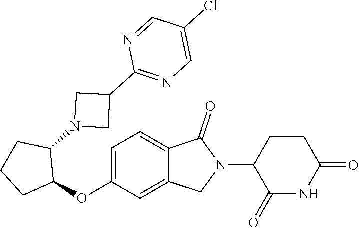 [SMILES: O=C1CCC(N2Cc3cc(O[C@H]4CCC[C@@H]4N4CC(c5ncc(Cl)cn5)C4)ccc3C2=O)C(=O)N1]

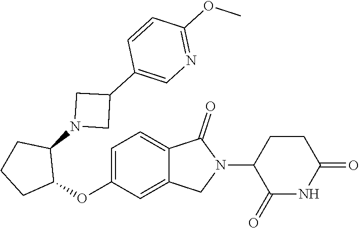 [SMILES: COc1ccc(C2CN([C@@H]3CCC[C@H]3Oc3ccc4c(c3)CN(C3CCC(=O)NC3=O)C4=O)C2)cn1]

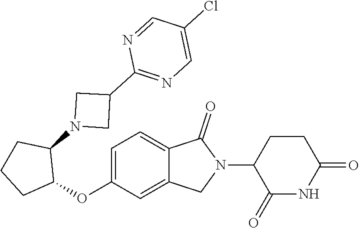 [SMILES: O=C1CCC(N2Cc3cc(O[C@@H]4CCC[C@H]4N4CC(c5ncc(Cl)cn5)C4)ccc3C2=O)C(=O)N1]

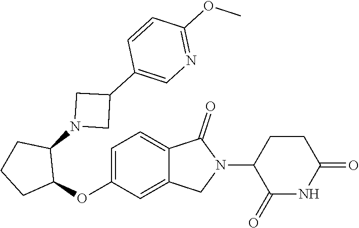 [SMILES: COc1ccc(C2CN([C@@H]3CCC[C@@H]3Oc3ccc4c(c3)CN(C3CCC(=O)NC3=O)C4=O)C2)cn1]

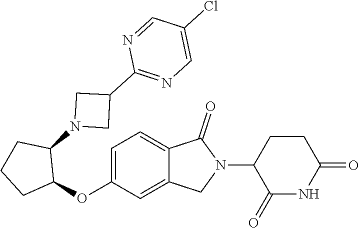 [SMILES: O=C1CCC(N2Cc3cc(O[C@H]4CCC[C@H]4N4CC(c5ncc(Cl)cn5)C4)ccc3C2=O)C(=O)N1]

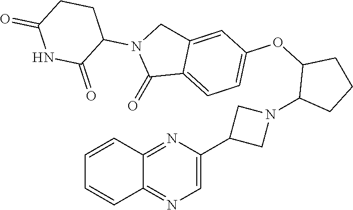 [SMILES: O=C1CCC(N2Cc3cc(OC4CCCC4N4CC(c5cnc6ccccc6n5)C4)ccc3C2=O)C(=O)N1]

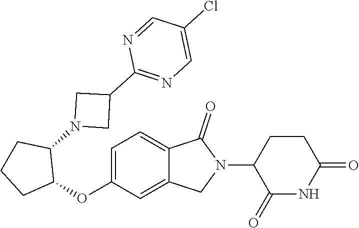 [SMILES: O=C1CCC(N2Cc3cc(O[C@@H]4CCC[C@@H]4N4CC(c5ncc(Cl)cn5)C4)ccc3C2=O)C(=O)N1]

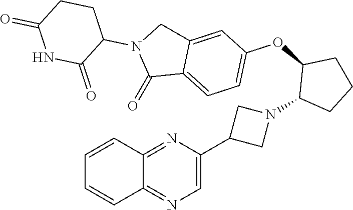 [SMILES: O=C1CCC(N2Cc3cc(O[C@H]4CCC[C@@H]4N4CC(c5cnc6ccccc6n5)C4)ccc3C2=O)C(=O)N1]

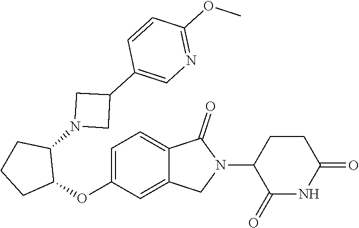 [SMILES: COc1ccc(C2CN([C@H]3CCC[C@H]3Oc3ccc4c(c3)CN(C3CCC(=O)NC3=O)C4=O)C2)cn1]

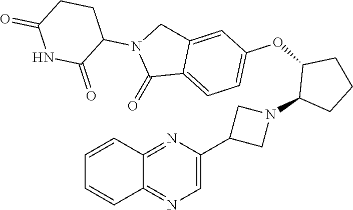 [SMILES: O=C1CCC(N2Cc3cc(O[C@@H]4CCC[C@H]4N4CC(c5cnc6ccccc6n5)C4)ccc3C2=O)C(=O)N1]

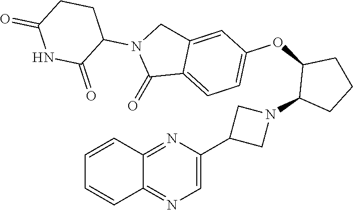 [SMILES: O=C1CCC(N2Cc3cc(O[C@H]4CCC[C@H]4N4CC(c5cnc6ccccc6n5)C4)ccc3C2=O)C(=O)N1]

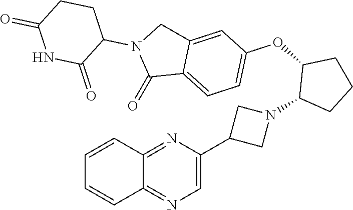 [SMILES: O=C1CCC(N2Cc3cc(O[C@@H]4CCC[C@@H]4N4CC(c5cnc6ccccc6n5)C4)ccc3C2=O)C(=O)N1]

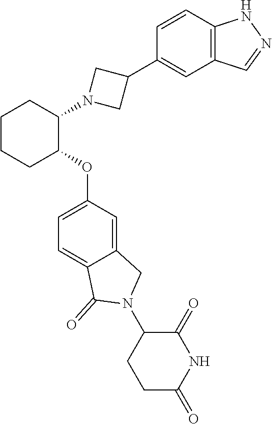 [SMILES: O=C1CCC(N2Cc3cc(O[C@@H]4CCCC[C@@H]4N4CC(c5ccc6[nH]ncc6c5)C4)ccc3C2=O)C(=O)N1]

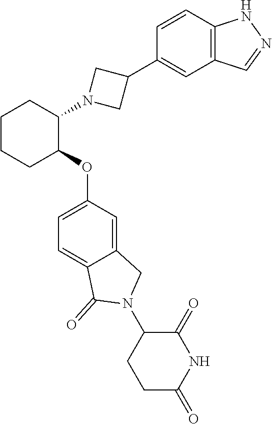 [SMILES: O=C1CCC(N2Cc3cc(O[C@H]4CCCC[C@@H]4N4CC(c5ccc6[nH]ncc6c5)C4)ccc3C2=O)C(=O)N1]

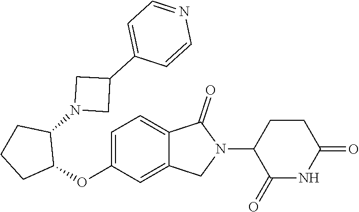 [SMILES: O=C1CCC(N2Cc3cc(O[C@@H]4CCC[C@@H]4N4CC(c5ccncc5)C4)ccc3C2=O)C(=O)N1]

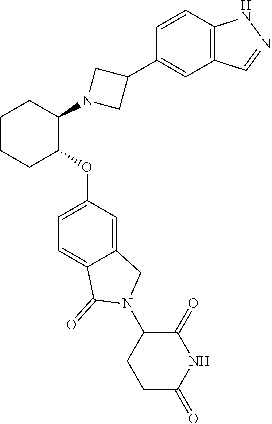 [SMILES: O=C1CCC(N2Cc3cc(O[C@@H]4CCCC[C@H]4N4CC(c5ccc6[nH]ncc6c5)C4)ccc3C2=O)C(=O)N1]

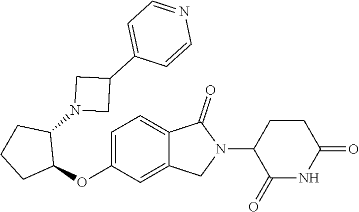 [SMILES: O=C1CCC(N2Cc3cc(O[C@H]4CCC[C@@H]4N4CC(c5ccncc5)C4)ccc3C2=O)C(=O)N1]

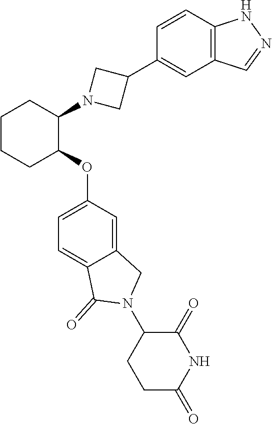 [SMILES: O=C1CCC(N2Cc3cc(O[C@H]4CCCC[C@H]4N4CC(c5ccc6[nH]ncc6c5)C4)ccc3C2=O)C(=O)N1]

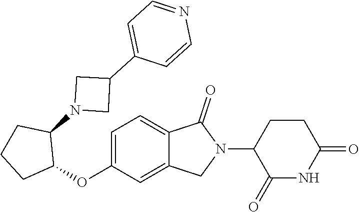 [SMILES: O=C1CCC(N2Cc3cc(O[C@@H]4CCC[C@H]4N4CC(c5ccncc5)C4)ccc3C2=O)C(=O)N1]

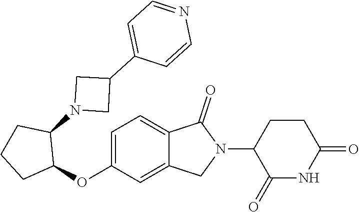 [SMILES: O=C1CCC(N2Cc3cc(O[C@H]4CCC[C@H]4N4CC(c5ccncc5)C4)ccc3C2=O)C(=O)N1]

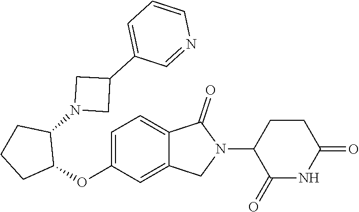 [SMILES: O=C1CCC(N2Cc3cc(O[C@@H]4CCC[C@@H]4N4CC(c5cccnc5)C4)ccc3C2=O)C(=O)N1]

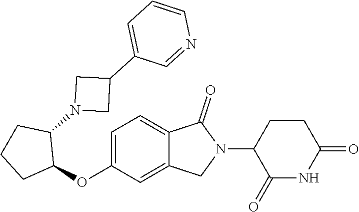 [SMILES: O=C1CCC(N2Cc3cc(O[C@H]4CCC[C@@H]4N4CC(c5cccnc5)C4)ccc3C2=O)C(=O)N1]

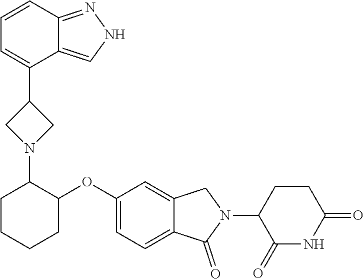 [SMILES: O=C1CCC(N2Cc3cc(OC4CCCCC4N4CC(c5cccc6n[nH]cc56)C4)ccc3C2=O)C(=O)N1]

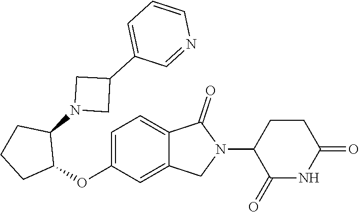 [SMILES: O=C1CCC(N2Cc3cc(O[C@@H]4CCC[C@H]4N4CC(c5cccnc5)C4)ccc3C2=O)C(=O)N1]

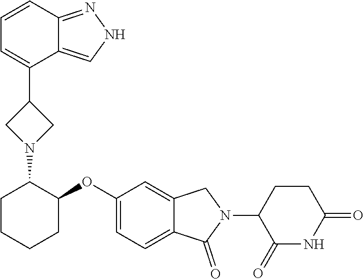 [SMILES: O=C1CCC(N2Cc3cc(O[C@H]4CCCC[C@@H]4N4CC(c5cccc6n[nH]cc56)C4)ccc3C2=O)C(=O)N1]

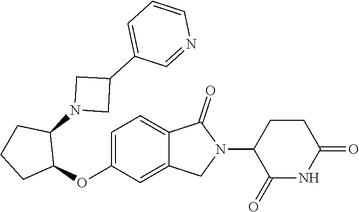 [SMILES: O=C1CCC(N2Cc3cc(O[C@H]4CCC[C@H]4N4CC(c5cccnc5)C4)ccc3C2=O)C(=O)N1]

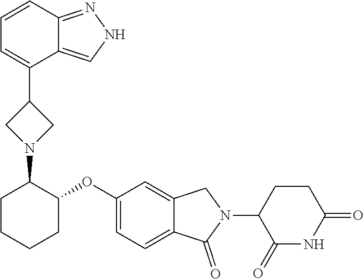 [SMILES: O=C1CCC(N2Cc3cc(O[C@@H]4CCCC[C@H]4N4CC(c5cccc6n[nH]cc56)C4)ccc3C2=O)C(=O)N1]

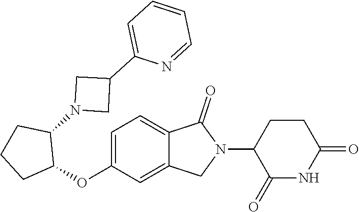 [SMILES: O=C1CCC(N2Cc3cc(O[C@@H]4CCC[C@@H]4N4CC(c5ccccn5)C4)ccc3C2=O)C(=O)N1]

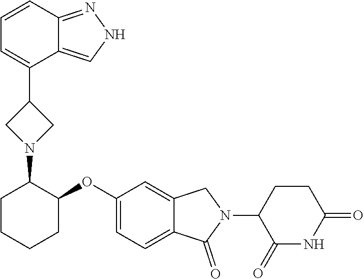 [SMILES: O=C1CCC(N2Cc3cc(O[C@H]4CCCC[C@H]4N4CC(c5cccc6n[nH]cc56)C4)ccc3C2=O)C(=O)N1]

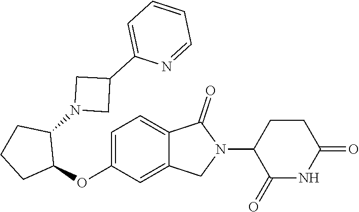 [SMILES: O=C1CCC(N2Cc3cc(O[C@H]4CCC[C@@H]4N4CC(c5ccccn5)C4)ccc3C2=O)C(=O)N1]

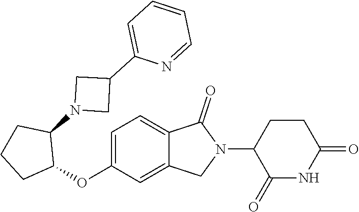 [SMILES: O=C1CCC(N2Cc3cc(O[C@@H]4CCC[C@H]4N4CC(c5ccccn5)C4)ccc3C2=O)C(=O)N1]

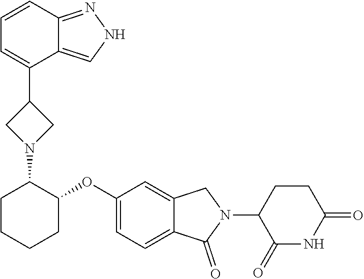 [SMILES: O=C1CCC(N2Cc3cc(O[C@@H]4CCCC[C@@H]4N4CC(c5cccc6n[nH]cc56)C4)ccc3C2=O)C(=O)N1]

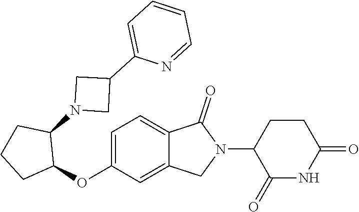 [SMILES: O=C1CCC(N2Cc3cc(O[C@H]4CCC[C@H]4N4CC(c5ccccn5)C4)ccc3C2=O)C(=O)N1]

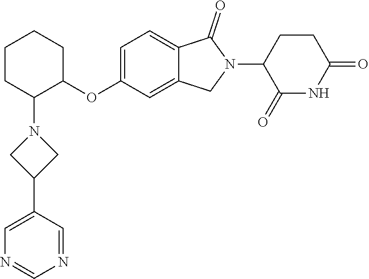 [SMILES: O=C1CCC(N2Cc3cc(OC4CCCCC4N4CC(c5cncnc5)C4)ccc3C2=O)C(=O)N1]

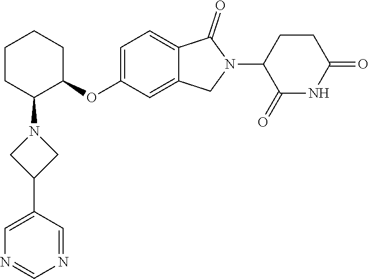 [SMILES: O=C1CCC(N2Cc3cc(O[C@@H]4CCCC[C@@H]4N4CC(c5cncnc5)C4)ccc3C2=O)C(=O)N1]

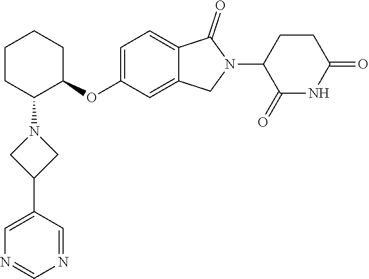 [SMILES: O=C1CCC(N2Cc3cc(O[C@@H]4CCCC[C@H]4N4CC(c5cncnc5)C4)ccc3C2=O)C(=O)N1]

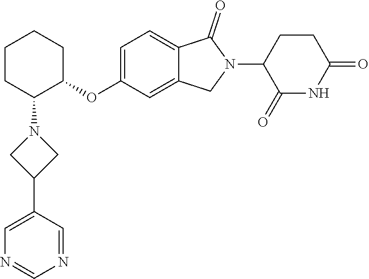 [SMILES: O=C1CCC(N2Cc3cc(O[C@H]4CCCC[C@H]4N4CC(c5cncnc5)C4)ccc3C2=O)C(=O)N1]

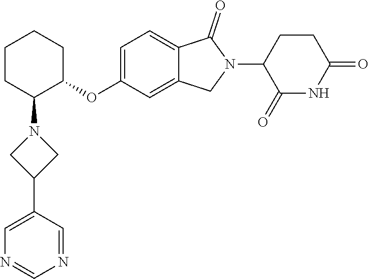 [SMILES: O=C1CCC(N2Cc3cc(O[C@H]4CCCC[C@@H]4N4CC(c5cncnc5)C4)ccc3C2=O)C(=O)N1]

In some embodiments, provided herein is a compound which degrades IKZF2 selected from Table 11B, or a pharmaceutically acceptable salt, solvate, stereoisomer, and/or tautomer thereof.

TABLE 1B

| # | Structure |
|---|---|
| 1-A | 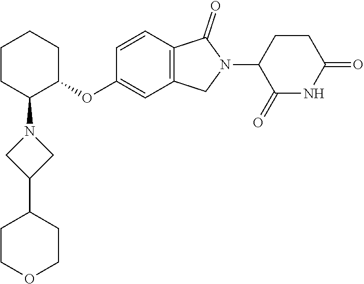 [SMILES: O=C1CCC(N2Cc3cc(O[C@H]4CCCC[C@@H]4N4CC(C5CCOCC5)C4)ccc3C2=O)C(=O)N1] |
| 12-A | 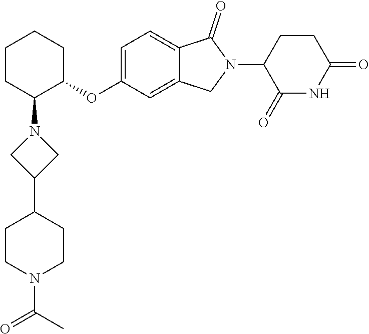 [SMILES: CC(=O)N1CCC(C2CN([C@H]3CCCC[C@@H]3Oc3ccc4c(c3)CN(C3CCC(=O)NC3=O)C4=O)C2)CC1] |
| 14-A | 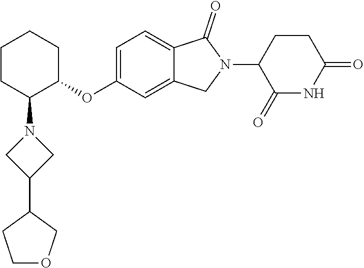 [SMILES: O=C1CCC(N2Cc3cc(O[C@H]4CCCC[C@@H]4N4CC(C5CCOC5)C4)ccc3C2=O)C(=O)N1] |
| 22-A | 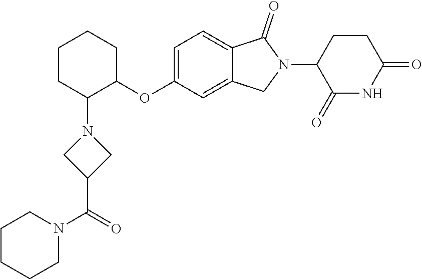 [SMILES: O=C1CCC(N2Cc3cc(OC4CCCCC4N4CC(C(=O)N5CCCCC5)C4)ccc3C2=O)C(=O)N1] |
| | Second Eluting Isomer |
| 24-A | 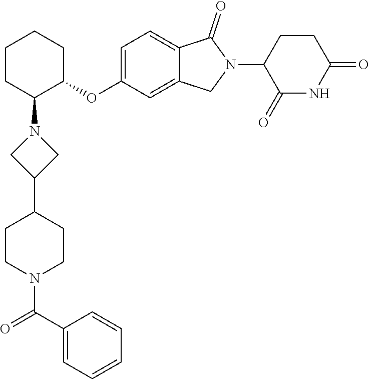 [SMILES: O=C1CCC(N2Cc3cc(O[C@H]4CCCC[C@@H]4N4CC(C5CCN(C(=O)c6ccccc6)CC5)C4)ccc3C2=O)C(=O)N1] |
| 25-A | 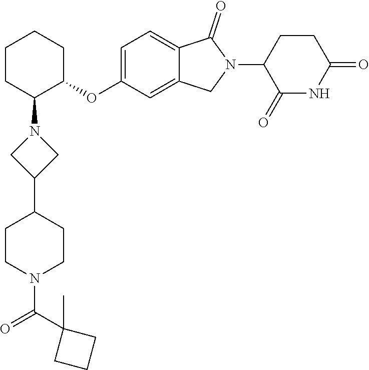 [SMILES: CC1(C(=O)N2CCC(C3CN([C@H]4CCCC[C@@H]4Oc4ccc5c(c4)CN(C4CCC(=O)NC4=O)C5=O)C3)CC2)CCC1] |
| 26-A | 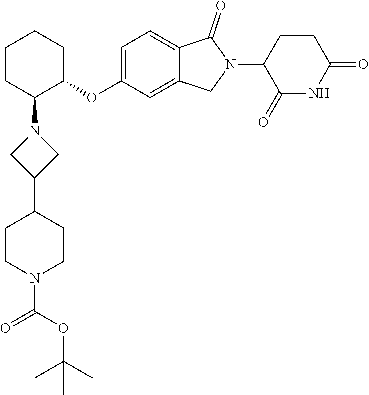 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1] |
| 27-A | 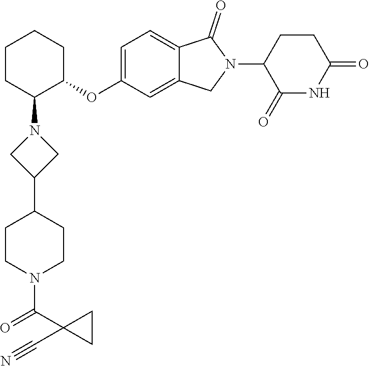 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1] |
| 28-A | 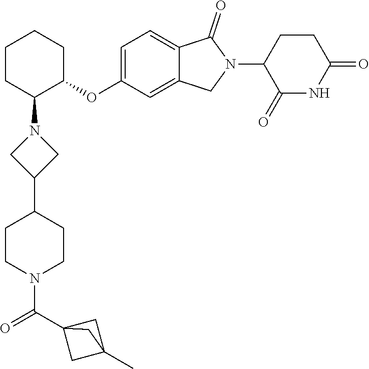 [SMILES: CC12CC(C(=O)N3CCC(C4CN([C@H]5CCCC[C@@H]5Oc5ccc6c(c5)CN(C5CCC(=O)NC5=O)C6=O)C4)CC3)(C1)C2] |
| 29-A | 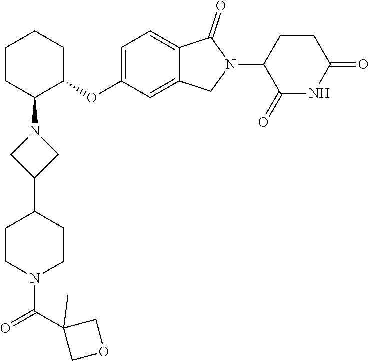 [SMILES: CC1(C(=O)N2CCC(C3CN([C@H]4CCCC[C@@H]4Oc4ccc5c(c4)CN(C4CCC(=O)NC4=O)C5=O)C3)CC2)COC1] |
| 30-A | 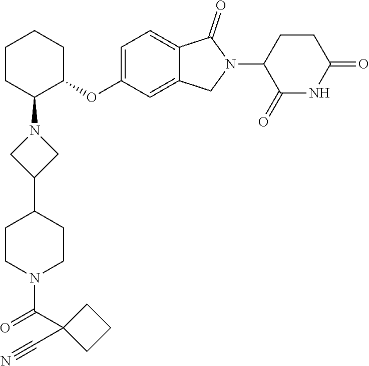 [SMILES: N#CC1(C(=O)N2CCC(C3CN([C@H]4CCCC[C@@H]4Oc4ccc5c(c4)CN(C4CCC(=O)NC4=O)C5=O)C3)CC2)CCC1] |
| 31-A | 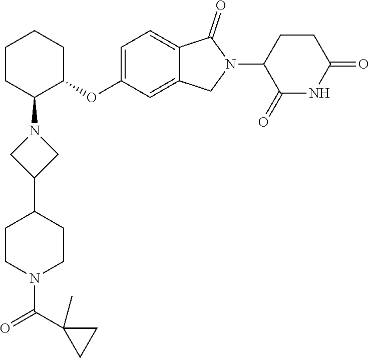 [SMILES: CC1(C(=O)N2CCC(C3CN([C@H]4CCCC[C@@H]4Oc4ccc5c(c4)CN(C4CCC(=O)NC4=O)C5=O)C3)CC2)CC1] |
| 32-A | 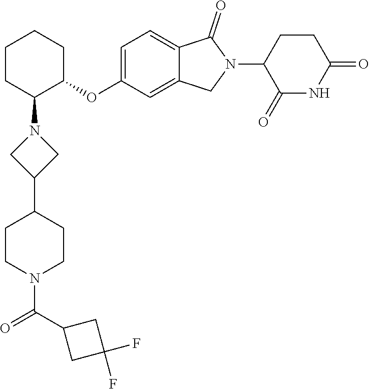 [SMILES: O=C1CCC(N2Cc3cc(O[C@H]4CCCC[C@@H]4N4CC(C5CCN(C(=O)C6CC(F)(F)C6)CC5)C4)ccc3C2=O)C(=O)N1] |
| 33-A | 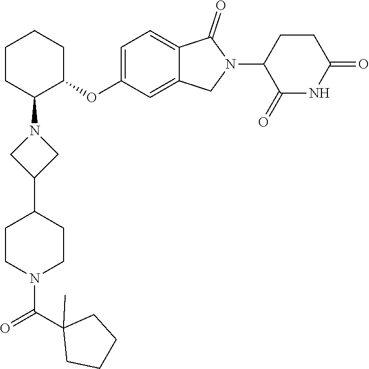 [SMILES: CC1(C(=O)N2CCC(C3CN([C@H]4CCCC[C@@H]4Oc4ccc5c(c4)CN(C4CCC(=O)NC4=O)C5=O)C3)CC2)CCCC1] |
| 34-A | 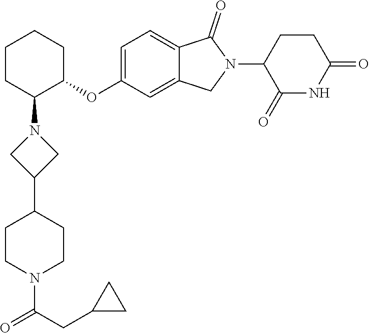 [SMILES: O=C1CCC(N2Cc3cc(O[C@H]4CCCC[C@@H]4N4CC(C5CCN(C(=O)CC6CC6)CC5)C4)ccc3C2=O)C(=O)N1] |
| 35-A | 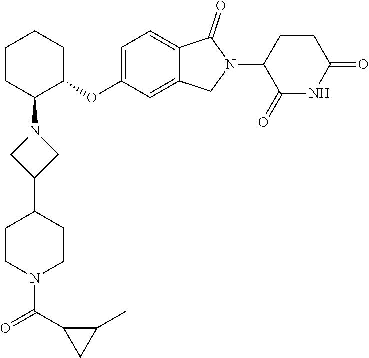 [SMILES: CC1CC1C(=O)N1CCC(C2CN([C@H]3CCCC[C@@H]3Oc3ccc4c(c3)CN(C3CCC(=O)NC3=O)C4=O)C2)CC1] |
| 41-A | 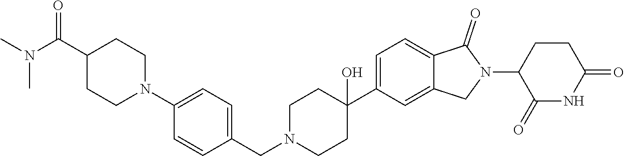 [SMILES: CN(C)C(=O)C1CCN(c2ccc(CN3CCC(O)(c4ccc5c(c4)CN(C4CCC(=O)NC4=O)C5=O)CC3)cc2)CC1] |
| 42-A | 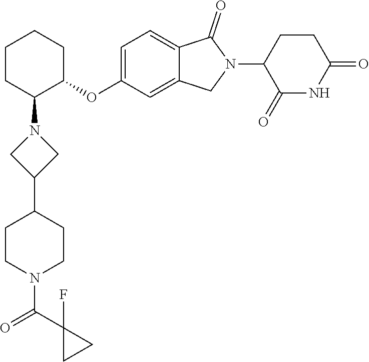 [SMILES: O=C1CCC(N2Cc3cc(O[C@H]4CCCC[C@@H]4N4CC(C5CCN(C(=O)C6(F)CC6)CC5)C4)ccc3C2=O)C(=O)N1] |
| 55-A | 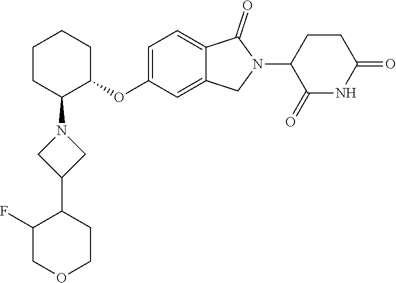 [SMILES: O=C1CCC(N2Cc3cc(O[C@H]4CCCC[C@@H]4N4CC(C5CCOCC5F)C4)ccc3C2=O)C(=O)N1] |
| 56-A | 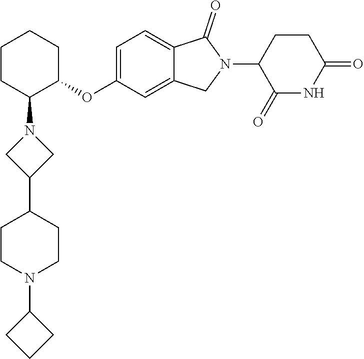 [SMILES: O=C1CCC(N2Cc3cc(O[C@H]4CCCC[C@@H]4N4CC(C5CCN(C6CCC6)CC5)C4)ccc3C2=O)C(=O)N1] |
| 57-A | 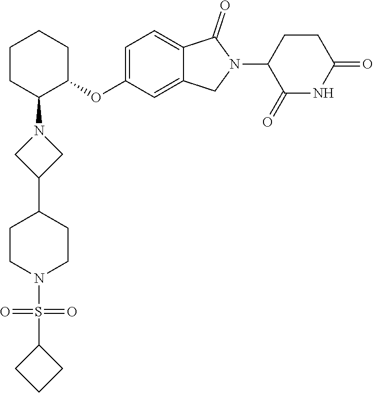 [SMILES: O=C1CCC(N2Cc3cc(O[C@H]4CCCC[C@@H]4N4CC(C5CCN(S(=O)(=O)C6CCC6)CC5)C4)ccc3C2=O)C(=O)N1] |
| 58-A | 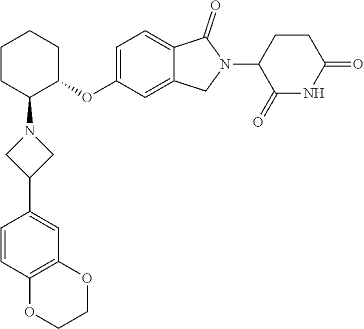 [SMILES: O=C1CCC(N2Cc3cc(O[C@H]4CCCC[C@@H]4N4CC(c5ccc6c(c5)OCCO6)C4)ccc3C2=O)C(=O)N1] |
| 1-B | 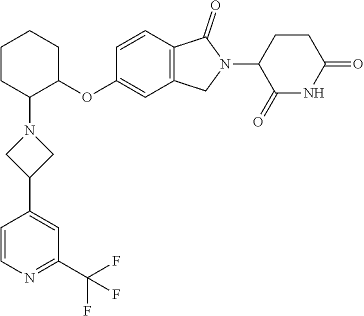 [SMILES: O=C1CCC(N2Cc3cc(OC4CCCCC4N4CC(c5ccnc(C(F)(F)F)c5)C4)ccc3C2=O)C(=O)N1] |
| 2-B | 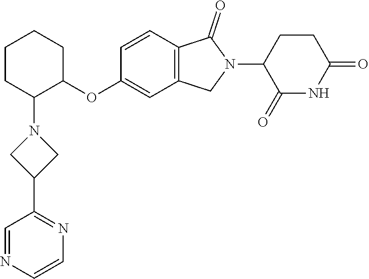 [SMILES: O=C1CCC(N2Cc3cc(OC4CCCCC4N4CC(c5cnccn5)C4)ccc3C2=O)C(=O)N1] |
| 6-B | 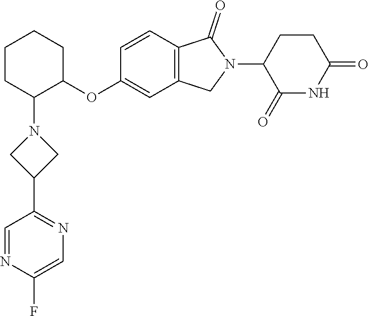 [SMILES: O=C1CCC(N2Cc3cc(OC4CCCCC4N4CC(c5cnc(F)cn5)C4)ccc3C2=O)C(=O)N1] |
| 11-B | 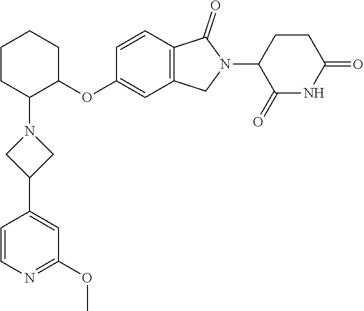 [SMILES: COc1cc(C2CN(C3CCCCC3Oc3ccc4c(c3)CN(C3CCC(=O)NC3=O)C4=O)C2)ccn1] |
| 16-B | 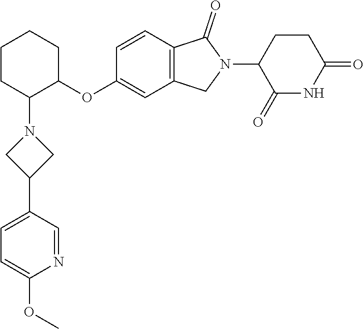 [SMILES: COc1ccc(C2CN(C3CCCCC3Oc3ccc4c(c3)CN(C3CCC(=O)NC3=O)C4=O)C2)cn1] |
| 21-B | 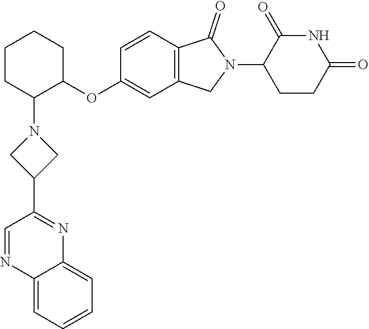 [SMILES: O=C1CCC(N2Cc3cc(OC4CCCCC4N4CC(c5cnc6ccccc6n5)C4)ccc3C2=O)C(=O)N1] |
| 26-B | 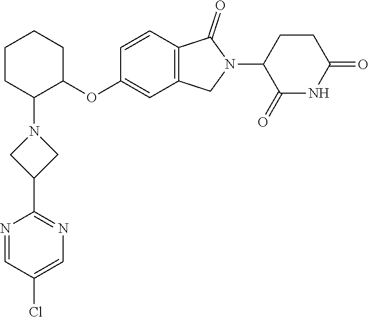 [SMILES: O=C1CCC(N2Cc3cc(OC4CCCCC4N4CC(c5ncc(Cl)cn5)C4)ccc3C2=O)C(=O)N1] |
| 31-B | 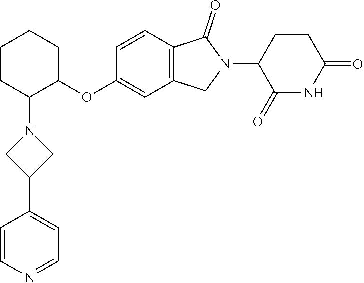 [SMILES: O=C1CCC(N2Cc3cc(OC4CCCCC4N4CC(c5ccncc5)C4)ccc3C2=O)C(=O)N1] |
| 36-B | 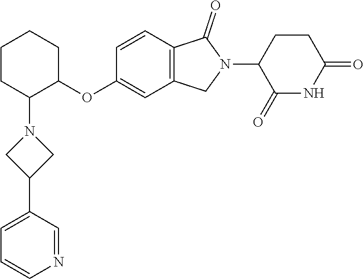 [SMILES: O=C1CCC(N2Cc3cc(OC4CCCCC4N4CC(c5cccnc5)C4)ccc3C2=O)C(=O)N1] |
| 46-B | 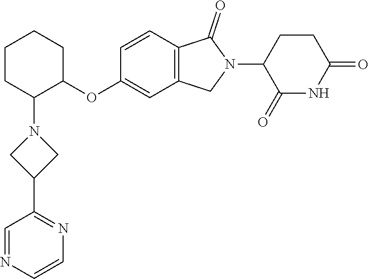 [SMILES: O=C1CCC(N2Cc3cc(OC4CCCCC4N4CC(c5cnccn5)C4)ccc3C2=O)C(=O)N1] |
| 56-B | 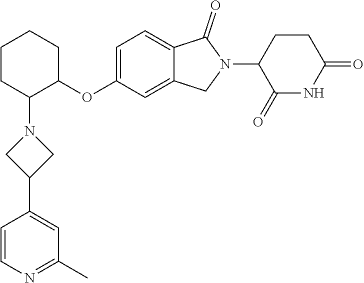 [SMILES: Cc1cc(C2CN(C3CCCCC3Oc3ccc4c(c3)CN(C3CCC(=O)NC3=O)C4=O)C2)ccn1] |
| 61-B | 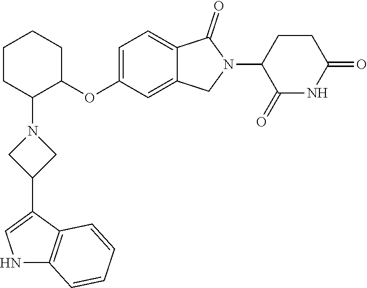 [SMILES: O=C1CCC(N2Cc3cc(OC4CCCCC4N4CC(c5c[nH]c6ccccc56)C4)ccc3C2=O)C(=O)N1] |
| 66-B | 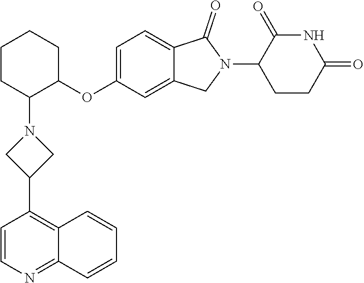 [SMILES: O=C1CCC(N2Cc3cc(OC4CCCCC4N4CC(c5ccnc6ccccc56)C4)ccc3C2=O)C(=O)N1] |
| 71-B | 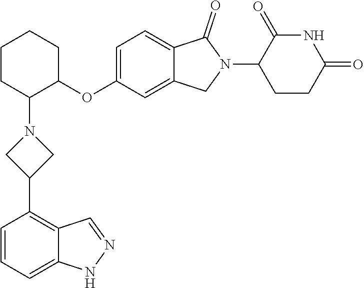 [SMILES: O=C1CCC(N2Cc3cc(OC4CCCCC4N4CC(c5cccc6[nH]ncc56)C4)ccc3C2=O)C(=O)N1] |
| 76-B | 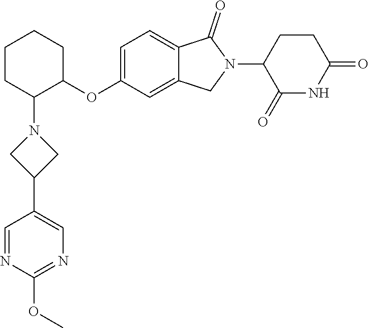 [SMILES: COc1ncc(C2CN(C3CCCCC3Oc3ccc4c(c3)CN(C3CCC(=O)NC3=O)C4=O)C2)cn1] |
| 81-B | 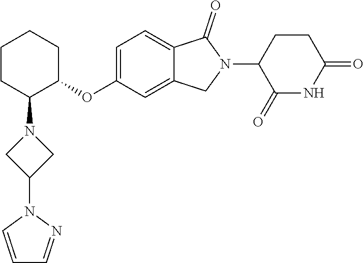 [SMILES: O=C1CCC(N2Cc3cc(O[C@H]4CCCC[C@@H]4N4CC(n5cccn5)C4)ccc3C2=O)C(=O)N1] |
| 87-B | 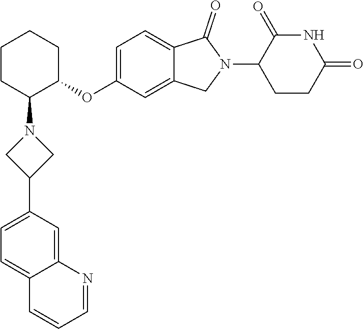 [SMILES: O=C1CCC(N2Cc3cc(O[C@H]4CCCC[C@@H]4N4CC(c5ccc6cccnc6c5)C4)ccc3C2=O)C(=O)N1] |
| 89-B | 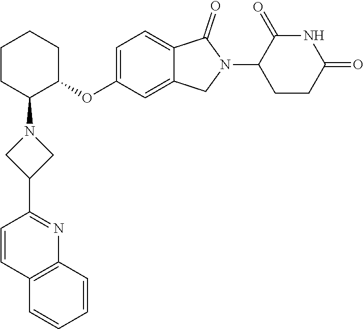 [SMILES: O=C1CCC(N2Cc3cc(O[C@H]4CCCC[C@@H]4N4CC(c5ccc6ccccc6n5)C4)ccc3C2=O)C(=O)N1] |
| | Diastereomer 1 |
| 90-B | 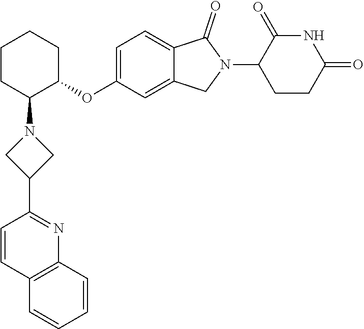 [SMILES: O=C1CCC(N2Cc3cc(O[C@H]4CCCC[C@@H]4N4CC(c5ccc6ccccc6n5)C4)ccc3C2=O)C(=O)N1] |
| | Diastereomer 2 |
| 91-B | 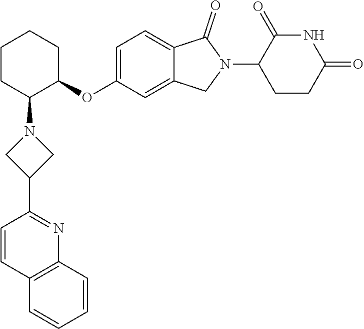 [SMILES: O=C1CCC(N2Cc3cc(O[C@@H]4CCCC[C@@H]4N4CC(c5ccc6ccccc6n5)C4)ccc3C2=O)C(=O)N1] |
| | Diastereomer 3 |
| 92-B | 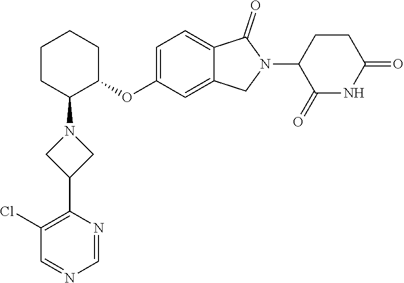 [SMILES: O=C1CCC(N2Cc3cc(O[C@H]4CCCC[C@@H]4N4CC(c5ncncc5Cl)C4)ccc3C2=O)C(=O)N1] |
| | Diastereomer 1 |
| 93-B | 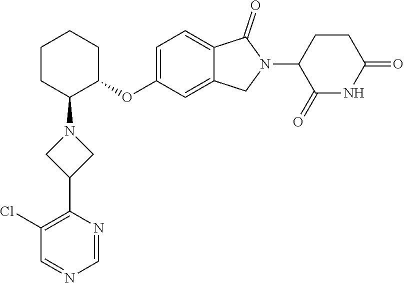 [SMILES: O=C1CCC(N2Cc3cc(O[C@H]4CCCC[C@@H]4N4CC(c5ncncc5Cl)C4)ccc3C2=O)C(=O)N1] |
| | Diastereomer 2 |
| 95-B | 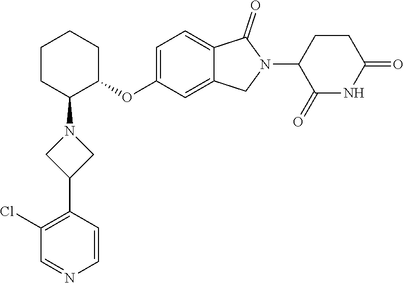 [SMILES: O=C1CCC(N2Cc3cc(O[C@H]4CCCC[C@@H]4N4CC(c5ccncc5Cl)C4)ccc3C2=O)C(=O)N1] |
| | Diastereomer 1 |
| 98-B | 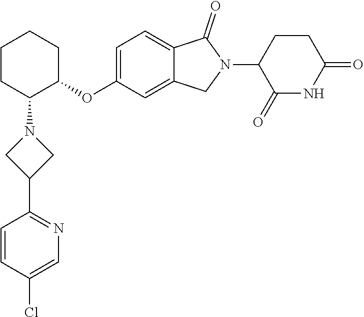 [SMILES: O=C1CCC(N2Cc3cc(O[C@H]4CCCC[C@H]4N4CC(c5ccc(Cl)cn5)C4)ccc3C2=O)C(=O)N1] |
| | Diastereomer 2 |
| 99-B | 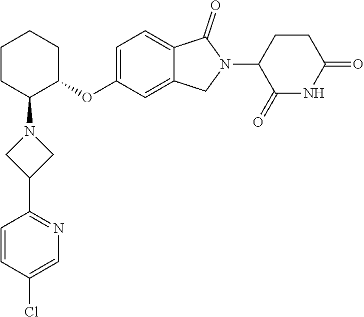 [SMILES: O=C1CCC(N2Cc3cc(O[C@H]4CCCC[C@@H]4N4CC(c5ccc(Cl)cn5)C4)ccc3C2=O)C(=O)N1] |
| | Diastereomer 3 |
| 100-B | 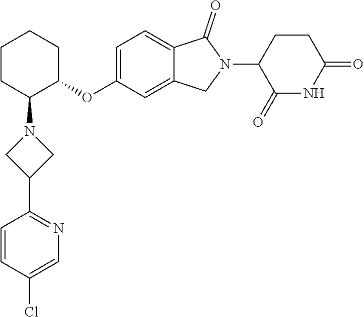 [SMILES: O=C1CCC(N2Cc3cc(O[C@H]4CCCC[C@@H]4N4CC(c5ccc(Cl)cn5)C4)ccc3C2=O)C(=O)N1] |
| | Diastereomer 4 |
| 102-B | 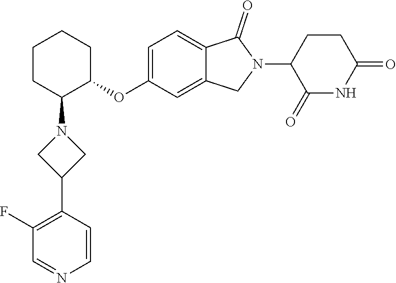 [SMILES: O=C1CCC(N2Cc3cc(O[C@H]4CCCC[C@@H]4N4CC(c5ccncc5F)C4)ccc3C2=O)C(=O)N1] |
| | Diastereomer 1 |
| 103-B | 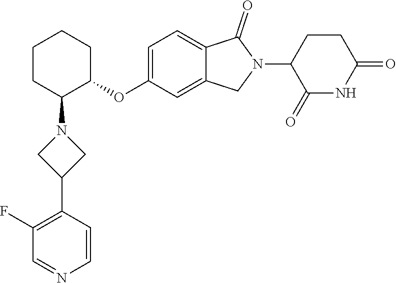 [SMILES: O=C1CCC(N2Cc3cc(O[C@H]4CCCC[C@@H]4N4CC(c5ccncc5F)C4)ccc3C2=O)C(=O)N1] |
| | Diastereomer 2 |
| 108-B | 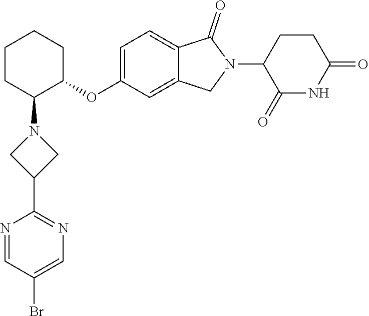 [SMILES: O=C1CCC(N2Cc3cc(O[C@H]4CCCC[C@@H]4N4CC(c5ncc(Br)cn5)C4)ccc3C2=O)C(=O)N1] |
| | Diastereomer 3 |
| 111-B | 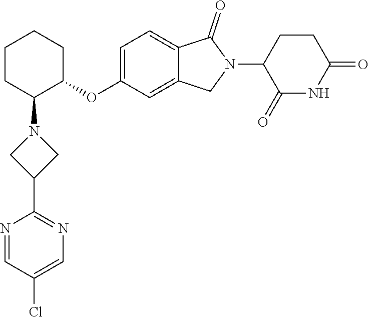 [SMILES: O=C1CCC(N2Cc3cc(O[C@H]4CCCC[C@@H]4N4CC(c5ncc(Cl)cn5)C4)ccc3C2=O)C(=O)N1] |
| | Diastereomer 3 |
| 112-B | 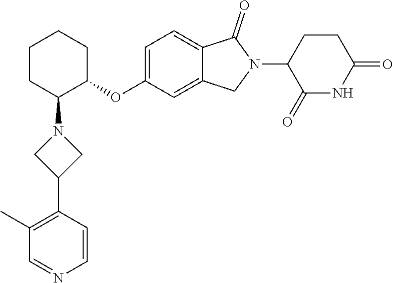 [SMILES: Cc1cnccc1C1CN([C@H]2CCCC[C@@H]2Oc2ccc3c(c2)CN(C2CCC(=O)NC2=O)C3=O)C1] |
| | Diastereomer 1 |
| 116-B | 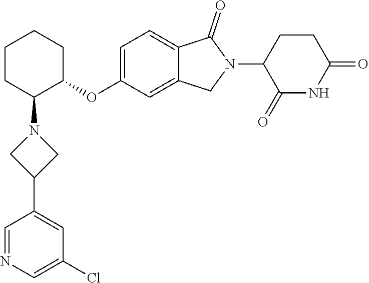 [SMILES: O=C1CCC(N2Cc3cc(O[C@H]4CCCC[C@@H]4N4CC(c5cncc(Cl)c5)C4)ccc3C2=O)C(=O)N1] |
| | Diastereomer 4 |
| 117-B | 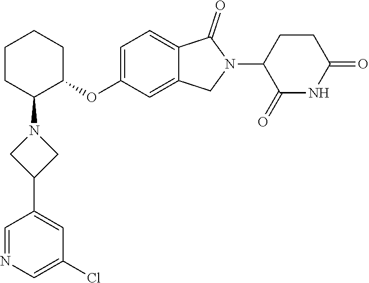 [SMILES: O=C1CCC(N2Cc3cc(O[C@H]4CCCC[C@@H]4N4CC(c5cncc(Cl)c5)C4)ccc3C2=O)C(=O)N1] |
| | Diastereomer 5 |
| 120-B | 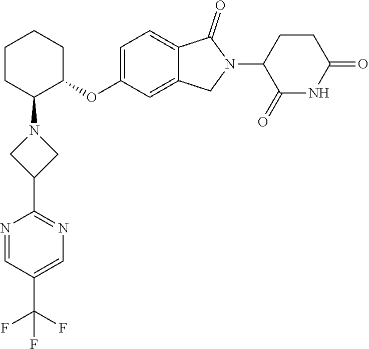 [SMILES: O=C1CCC(N2Cc3cc(O[C@H]4CCCC[C@@H]4N4CC(c5ncc(C(F)(F)F)cn5)C4)ccc3C2=O)C(=O)N1] |
| | Diastereomer 3 |
| 123-B | 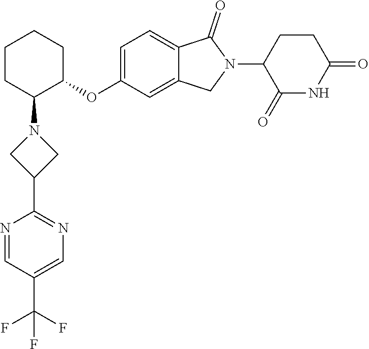 [SMILES: O=C1CCC(N2Cc3cc(O[C@H]4CCCC[C@@H]4N4CC(c5ncc(C(F)(F)F)cn5)C4)ccc3C2=O)C(=O)N1] |
| | Diastereomer 6 |
| 125-B | 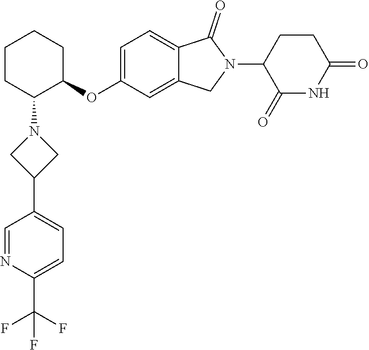 [SMILES: O=C1CCC(N2Cc3cc(O[C@@H]4CCCC[C@H]4N4CC(c5ccc(C(F)(F)F)nc5)C4)ccc3C2=O)C(=O)N1] |
| | Diastereomer 2 |
| 126-B | 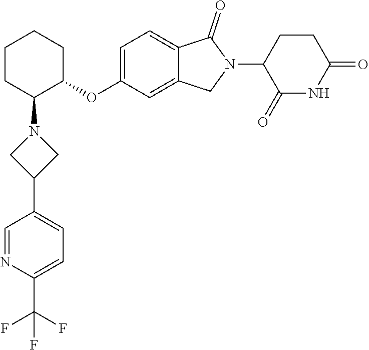 [SMILES: O=C1CCC(N2Cc3cc(O[C@H]4CCCC[C@@H]4N4CC(c5ccc(C(F)(F)F)nc5)C4)ccc3C2=O)C(=O)N1] |
| | Diastereomer 3 |
| 127-B | 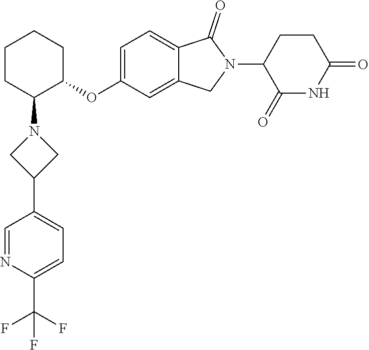 [SMILES: O=C1CCC(N2Cc3cc(O[C@H]4CCCC[C@@H]4N4CC(c5ccc(C(F)(F)F)nc5)C4)ccc3C2=O)C(=O)N1] |
| | Diastereomer 4 |
| 129-B | 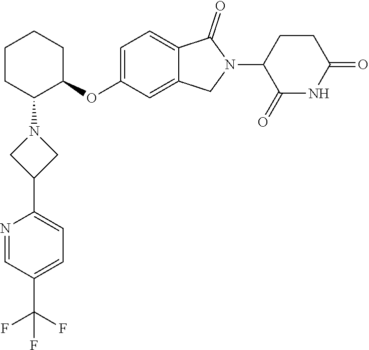 [SMILES: O=C1CCC(N2Cc3cc(O[C@@H]4CCCC[C@H]4N4CC(c5ccc(C(F)(F)F)cn5)C4)ccc3C2=O)C(=O)N1] |
| | Diastereomer 2 |
| 130-B | 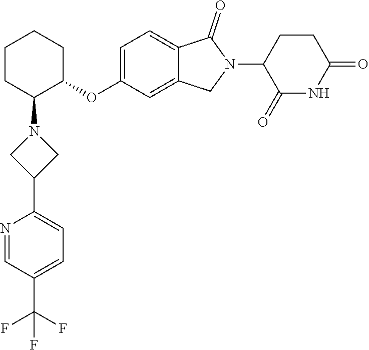 [SMILES: O=C1CCC(N2Cc3cc(O[C@H]4CCCC[C@@H]4N4CC(c5ccc(C(F)(F)F)cn5)C4)ccc3C2=O)C(=O)N1] |
| | Diastereomer 3 |
| 131-B | 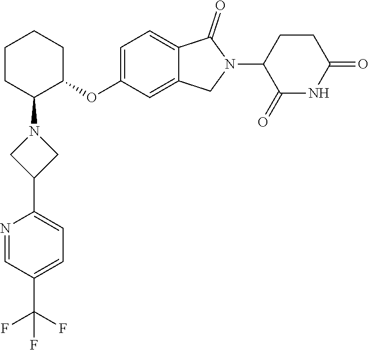 [SMILES: O=C1CCC(N2Cc3cc(O[C@H]4CCCC[C@@H]4N4CC(c5ccc(C(F)(F)F)cn5)C4)ccc3C2=O)C(=O)N1] |
| | Diastereomer 4 |
| 134-B | 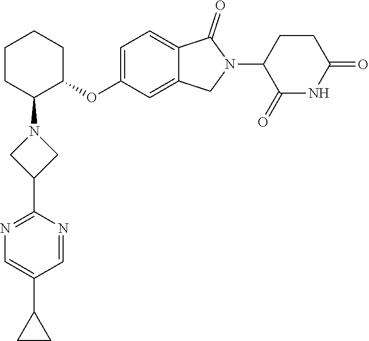 [SMILES: O=C1CCC(N2Cc3cc(O[C@H]4CCCC[C@@H]4N4CC(c5ncc(C6CC6)cn5)C4)ccc3C2=O)C(=O)N1] |
| | Diastereomer 3 |
| 135-B | 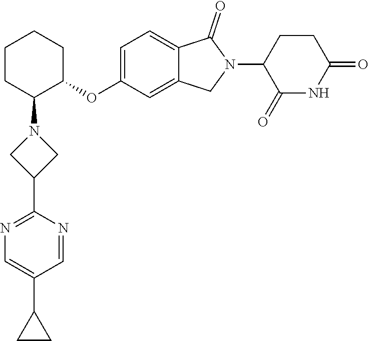 [SMILES: O=C1CCC(N2Cc3cc(O[C@H]4CCCC[C@@H]4N4CC(c5ncc(C6CC6)cn5)C4)ccc3C2=O)C(=O)N1] |
| | Diastereomer 4 |
| 136-B | 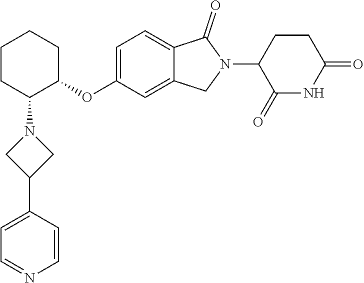 [SMILES: O=C1CCC(N2Cc3cc(O[C@H]4CCCC[C@H]4N4CC(c5ccncc5)C4)ccc3C2=O)C(=O)N1] |
| | Diastereomer 3 |
| 137-B | 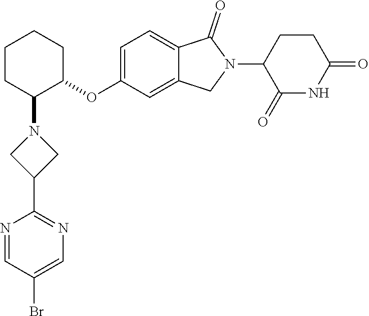 [SMILES: O=C1CCC(N2Cc3cc(O[C@H]4CCCC[C@@H]4N4CC(c5ncc(Br)cn5)C4)ccc3C2=O)C(=O)N1] |
| 139-B | 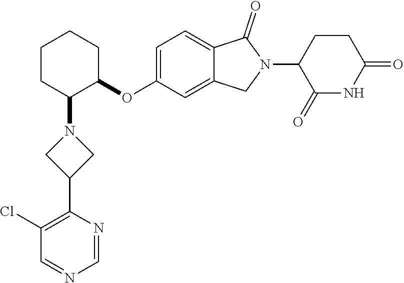 [SMILES: O=C1CCC(N2Cc3cc(O[C@@H]4CCCC[C@@H]4N4CC(c5ncncc5Cl)C4)ccc3C2=O)C(=O)N1] |
| 142-B | 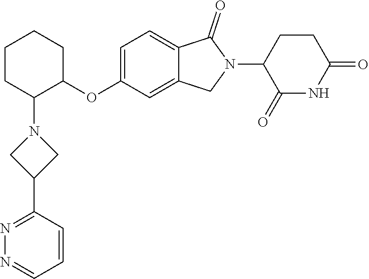 [SMILES: O=C1CCC(N2Cc3cc(OC4CCCCC4N4CC(c5cccnn5)C4)ccc3C2=O)C(=O)N1] |
| 146-B | 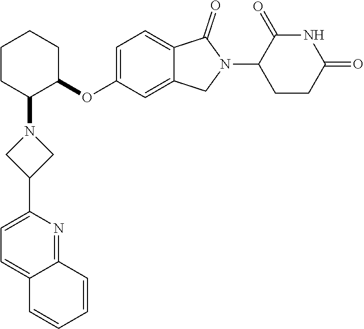 [SMILES: O=C1CCC(N2Cc3cc(O[C@@H]4CCCC[C@@H]4N4CC(c5ccc6ccccc6n5)C4)ccc3C2=O)C(=O)N1] |
| 152-B | 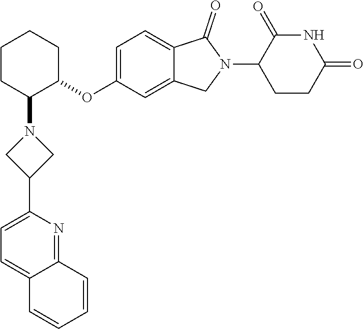 [SMILES: O=C1CCC(N2Cc3cc(O[C@H]4CCCC[C@@H]4N4CC(c5ccc6ccccc6n5)C4)ccc3C2=O)C(=O)N1] |
| 153-B | 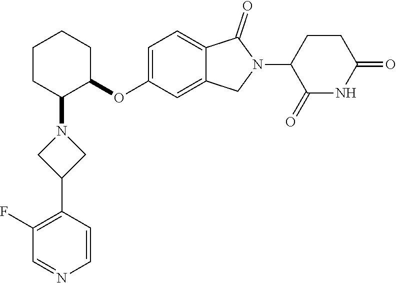 [SMILES: O=C1CCC(N2Cc3cc(O[C@@H]4CCCC[C@@H]4N4CC(c5ccncc5F)C4)ccc3C2=O)C(=O)N1] |
| 155-B | 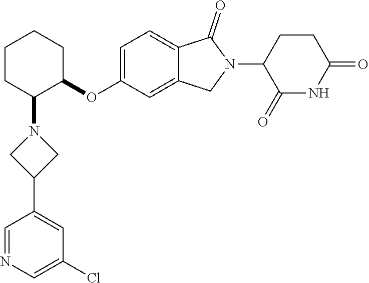 [SMILES: O=C1CCC(N2Cc3cc(O[C@@H]4CCCC[C@@H]4N4CC(c5cncc(Cl)c5)C4)ccc3C2=O)C(=O)N1] |
| 161-B | 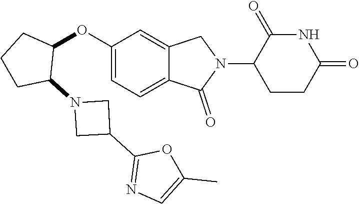 |
| 162-B | 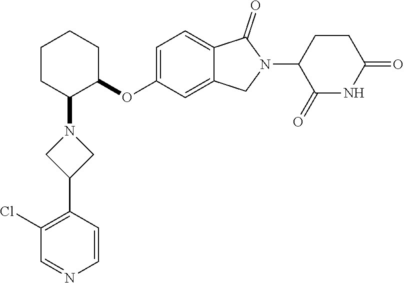 [SMILES: O=C1CCC(N2Cc3cc(O[C@@H]4CCCC[C@@H]4N4CC(c5ccncc5Cl)C4)ccc3C2=O)C(=O)N1] |
| 163-B | 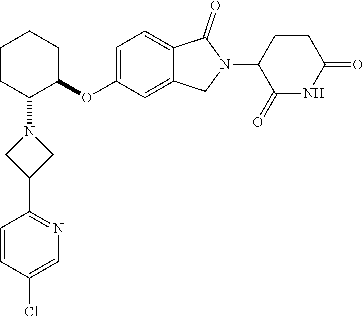 [SMILES: O=C1CCC(N2Cc3cc(O[C@@H]4CCCC[C@H]4N4CC(c5ccc(Cl)cn5)C4)ccc3C2=O)C(=O)N1] |
| 165-B | 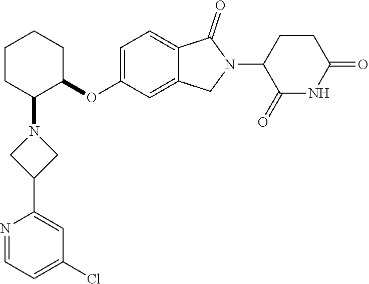 [SMILES: O=C1CCC(N2Cc3cc(O[C@@H]4CCCC[C@@H]4N4CC(c5cc(Cl)ccn5)C4)ccc3C2=O)C(=O)N1] |
| 169-B | 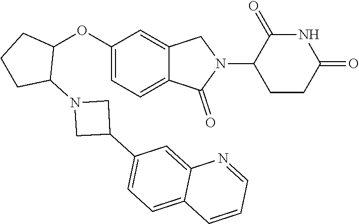 [SMILES: O=C1CCC(N2Cc3cc(OC4CCCC4N4CC(c5ccc6cccnc6c5)C4)ccc3C2=O)C(=O)N1] |
| 170-B | 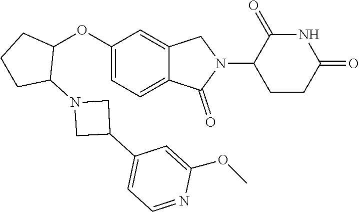 [SMILES: COc1cc(C2CN(C3CCCC3Oc3ccc4c(c3)CN(C3CCC(=O)NC3=O)C4=O)C2)ccn1] |
| 173-B | 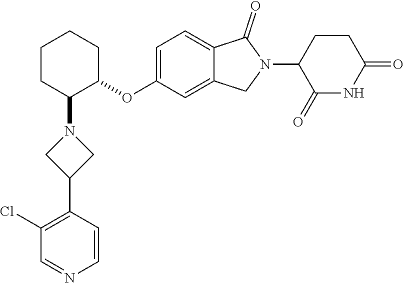 [SMILES: O=C1CCC(N2Cc3cc(O[C@H]4CCCC[C@@H]4N4CC(c5ccncc5Cl)C4)ccc3C2=O)C(=O)N1] |
| 174-B | 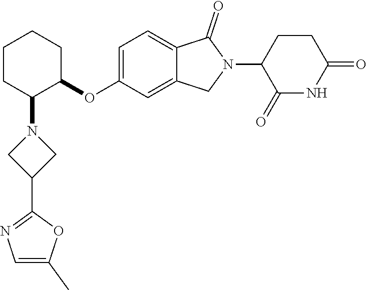 [SMILES: Cc1cnc(C2CN([C@H]3CCCC[C@H]3Oc3ccc4c(c3)CN(C3CCC(=O)NC3=O)C4=O)C2)o1] |
| 177-B | 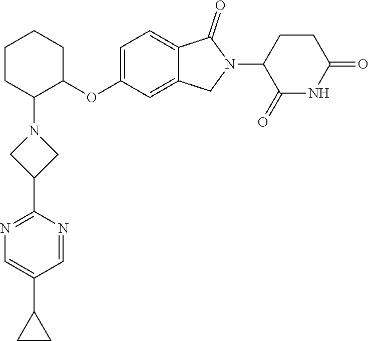 [SMILES: O=C1CCC(N2Cc3cc(OC4CCCCC4N4CC(c5ncc(C6CC6)cn5)C4)ccc3C2=O)C(=O)N1] |
| 179-B | 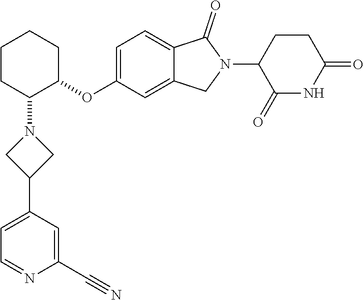 [SMILES: N#Cc1cc(C2CN([C@@H]3CCCC[C@@H]3Oc3ccc4c(c3)CN(C3CCC(=O)NC3=O)C4=O)C2)ccn1] |
| 182-B | 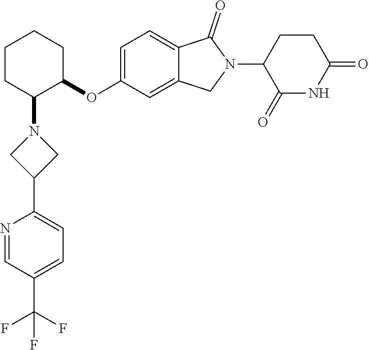 [SMILES: O=C1CCC(N2Cc3cc(O[C@@H]4CCCC[C@@H]4N4CC(c5ccc(C(F)(F)F)cn5)C4)ccc3C2=O)C(=O)N1] |
| 183-B | 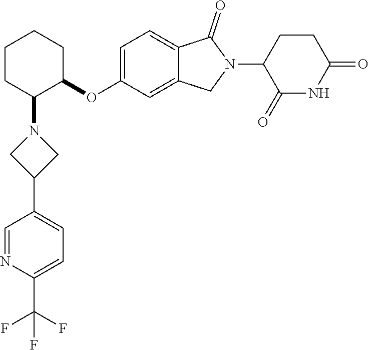 [SMILES: O=C1CCC(N2Cc3cc(O[C@@H]4CCCC[C@@H]4N4CC(c5ccc(C(F)(F)F)nc5)C4)ccc3C2=O)C(=O)N1] |
| 184-B | 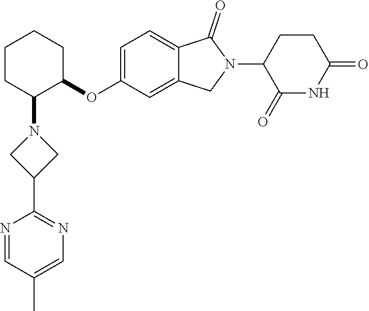 [SMILES: Cc1cnc(C2CN([C@H]3CCCC[C@H]3Oc3ccc4c(c3)CN(C3CCC(=O)NC3=O)C4=O)C2)nc1] |
| 185-B | 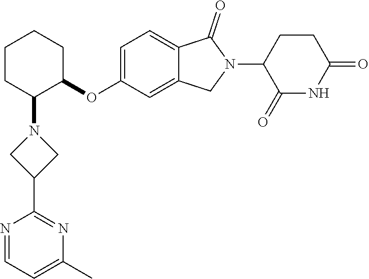 [SMILES: Cc1ccnc(C2CN([C@H]3CCCC[C@H]3Oc3ccc4c(c3)CN(C3CCC(=O)NC3=O)C4=O)C2)n1] |
| 186-B | 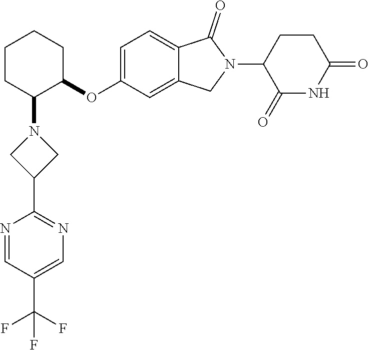 [SMILES: O=C1CCC(N2Cc3cc(O[C@@H]4CCCC[C@@H]4N4CC(c5ncc(C(F)(F)F)cn5)C4)ccc3C2=O)C(=O)N1] |
| 188-B | 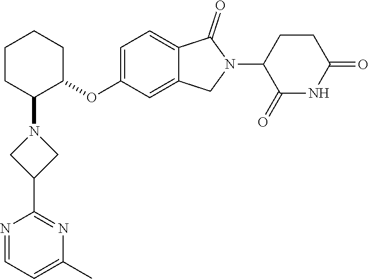 [SMILES: Cc1ccnc(C2CN([C@H]3CCCC[C@@H]3Oc3ccc4c(c3)CN(C3CCC(=O)NC3=O)C4=O)C2)n1] |
| 190-B | 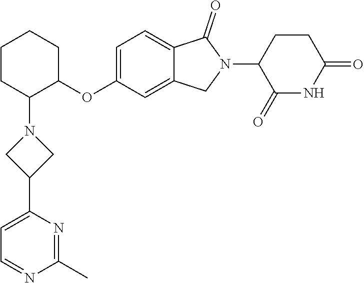 [SMILES: Cc1nccc(C2CN(C3CCCCC3Oc3ccc4c(c3)CN(C3CCC(=O)NC3=O)C4=O)C2)n1] |
| 193-B | 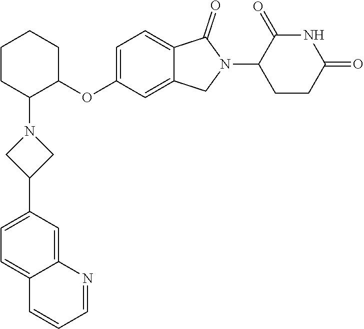 [SMILES: O=C1CCC(N2Cc3cc(OC4CCCCC4N4CC(c5ccc6cccnc6c5)C4)ccc3C2=O)C(=O)N1] |
| 194-B | 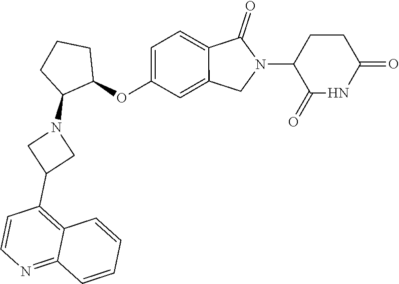 [SMILES: O=C1CCC(N2Cc3cc(O[C@@H]4CCC[C@@H]4N4CC(c5ccnc6ccccc56)C4)ccc3C2=O)C(=O)N1] |
| 198-B | 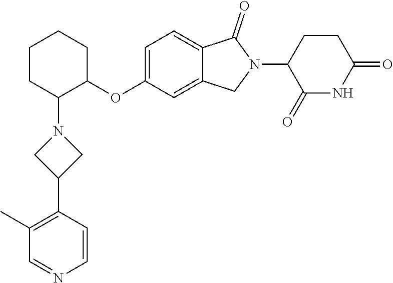 [SMILES: Cc1cnccc1C1CN(C2CCCCC2Oc2ccc3c(c2)CN(C2CCC(=O)NC2=O)C3=O)C1] |
| 199-B | 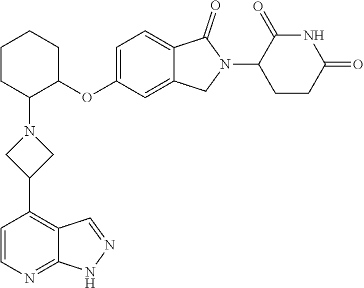 [SMILES: O=C1CCC(N2Cc3cc(OC4CCCCC4N4CC(c5ccnc6[nH]ncc56)C4)ccc3C2=O)C(=O)N1] |
| 201-B | 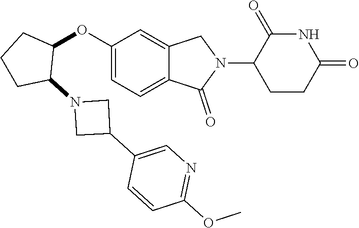 |
| 204-B | 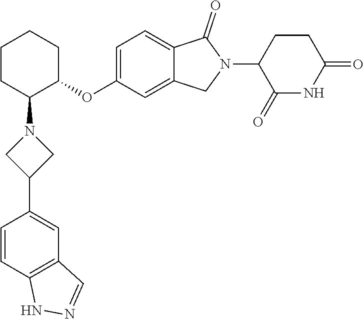 [SMILES: O=C1CCC(N2Cc3cc(O[C@H]4CCCC[C@@H]4N4CC(c5ccc6[nH]ncc6c5)C4)ccc3C2=O)C(=O)N1] |
| 205-B | 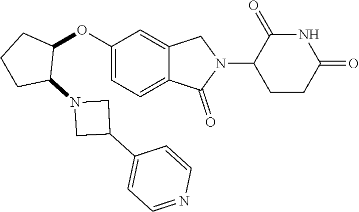 |
| 209-B | 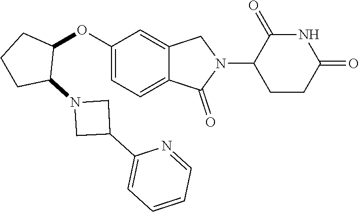 |
| 211-B | 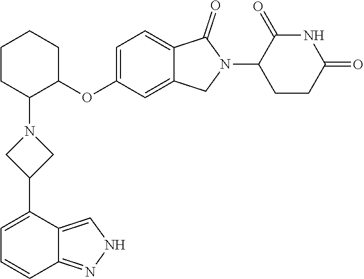 [SMILES: O=C1CCC(N2Cc3cc(OC4CCCCC4N4CC(c5cccc6n[nH]cc56)C4)ccc3C2=O)C(=O)N1] |

### General Synthetic Methods

The compounds of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, and VI-B described herein can be prepared from readily available starting materials using the following general methods and procedures. It will be appreciated that where typical process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

Additionally, as will be apparent to those skilled in the art, intermediate and final compounds obtained as enantiomeric mixtures may be separated into their separate enantiomers by liquid chromatography using a chiral stationary phase to give chiral selectivity. Suitable chiral stationary phases as well as suitable conditions for chiral separation are well known in the art. For example, numerous methods are described in F. Toda, Enantiomeric Separation: Fundamentals and Practical Methods, First Edition, Springer, Dordrecht, 2004, and references cited therein.

The starting materials for the following reactions are generally known compounds or can be prepared by known procedures or obvious modifications thereof. For example, many of the starting materials are available from commercial suppliers such as Sigma Aldrich (St. Louis, Missouri, USA), Bachem (Torrance, California, USA), Emka-Chemce (St. Louis, Missouri, USA). Others may be prepared by procedures, or obvious modifications thereof, described in standard reference texts such as Fieser and Fieser's Reagentsfor Organic Synthesis, Volumes 1-15 (John Wiley, and Sons, 2016), Rodd's Chemistry of Carbon Compounds, Volumes 1-5, and Supplementals (Elsevier Science Publishers, 2001), Organic Reactions, Volumes 1-40 (John Wiley, and Sons, 2019), March's Advanced Organic Chemistry, (John Wiley, and Sons, 8^{th} Edition, 2019), and Larock's Comprehensive Organic Transformations (VCH Publishers Inc., 1989).

### Synthesis of Representative Compounds

The general synthesis of the compounds described herein is set forth in the reaction schemes below. In the Schemes below, substituents R^{1′}, R^{2′}, R^{3′}, R^{4′}, R^{11′}, X′, Y′, Z′, Z^{1′}, m′, n′, p′, q′, s′ and t′ are as defined throughout the specification, and are used generally, such that:

R^{1′} is R^{1′}, R¹, or R⁴¹;

R^{2′} is R^{2′}, R², or R⁴²

R^{3′} is R^{3′}, R³, or R⁴³

R^{4′} is R^{4′}, R⁴, or R⁴⁴;

R^{11′} is R^{11′}, R¹¹, or R⁵¹;

X′ is X′, X, or X⁴⁰.

Y′ is Y′, Y, or Y⁴⁰;

Z′ is Z′, Z, or Z⁴⁰;

Z^{1′} is Z^{1′}, Z¹, or Z⁴¹;

m′ is m′, m, or m⁴⁰.

n′ is n′, n, or n⁴⁰;

p′ is p′, p, or p⁴⁰;

q′ is q′, q, or q⁴¹;

s′ is s′, s, or s⁴⁰;

t′ is t′, t, or t⁴⁰;

Q′ is a leaving group (including, but not limited to, Br, Cl, I, triflate, and the like).

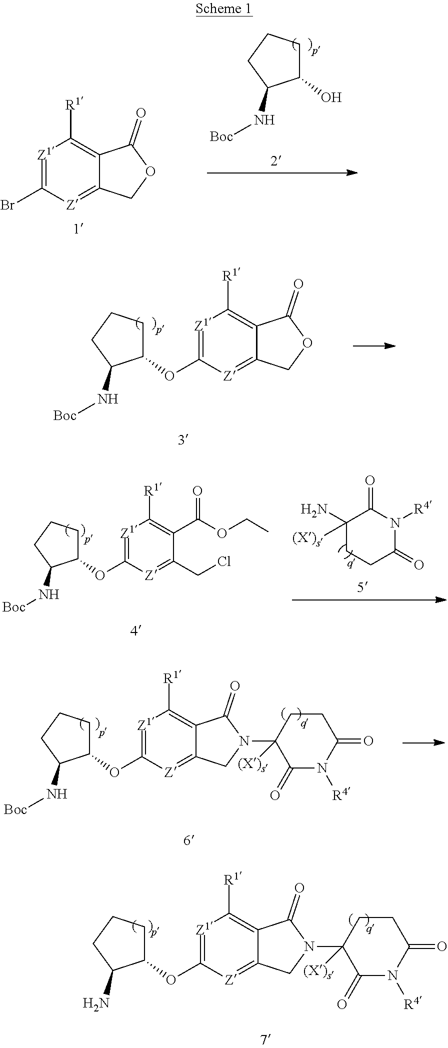

As to the reaction in Scheme 1, in the first step, at least a stoichiometric amount of protected amino alcohol), compound 2′, is combined with compound 1′, CAS #64169-34-2 (where R^{1′}=H; Z′ and Z^{1′} are each CH), in an inert diluent such as THF, MeCN, toluene and the like, typically in the presence of a suitable catalyst such as Ir, Cu(OAc)₂, SMI₂, and the like. The reaction is typically maintained at from 20° to 50° C. until it is substantially complete. Conventional workup of the reaction solution can be followed by isolation/purification processes such as crystallization, chromatography, high performance liquid chromatography (HPLC), and the like to provide for compound 3′.

In the next step, at least a stoichiometric equivalent of thionyl chloride is combined with compound 3′ in a diluent such as methanol, ethanol and the like. The reaction is typically maintained at from 50° to 80° C. until it is substantially complete. Conventional workup of the reaction solution can be followed by isolation/purification processes such as crystallization, chromatography, high performance liquid chromatography (HPLC), and the like to provide for compound 4′.

In the next step, at least a stoichiometric amount of 3-aminopiperidine-2,6-dione.hydrochloride, CAS #24666-56-6 (where R^{4′}═H; X′═H; q′=1; s′=1), compound 5′, is combined with compound 4′ in an inert diluent such as dichloromethane, tetrachloromethane and the like in the presence of a suitable base such as triethylamine, diisopropylethylamine, pyridine and the like. The reaction is typically maintained at from 0° C. to 30° C. until it is substantially complete. Conventional workup of the reaction solution can be followed by isolation/purification processes such as crystallization, chromatography, high performance liquid chromatography (HPLC), and the like, to provide for compound 6′.

In the final step, the t-butoxycarbonyl (BOC) protecting group is removed by conventional conditions. The BOC group is illustrative only and other conventional amino blocking groups such as benzyl, 9-fluorenylmethoxycarbonyl (Fmoc), benzyloxycarbonyl (Cbz), p-nitrobenzyloxycarbonyl and the like could be used. Upon reaction completion, conventional workup of the reaction solution can be followed by isolation/purification processes such as crystallization, chromatography, high performance liquid chromatography (HPLC), and the like to provide for compound 7′, which serves as an intermediate for the synthesis of compounds of formula I-A′, formula I-A, formula I-B, and sub-formulae thereof.

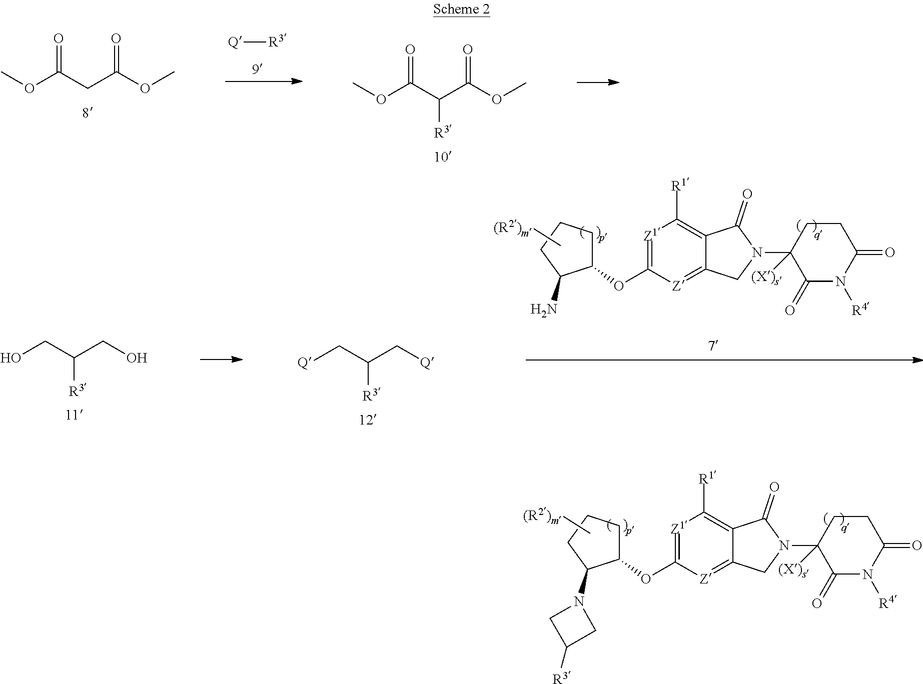

As to the reaction in Scheme 2, the first step is a conventional alkylation reaction wherein at least a stoichiometric equivalent of an alkylating reagent 9′ is combined with dimethylmalonate, compound 8′, in an inert diluent such as DMF, THF, MeCN and the like in the presence of a suitable base such as sodium hydride, LDA, n-BuLi, cesium carbonate and the like. The reaction is typically maintained at from 0° to 70° C. until it is substantially complete. Conventional workup of the reaction solution can be followed by isolation/purification processes such as crystallization, chromatography, high performance liquid chromatography (HPLC), and the like to provide for compound 10′.

In the next step, at least a stoichiometric amount of compound 10′, in an inert diluent such as THF, MeCN, toluene and the like in the presence of a suitable reducing reagent such as lithium aluminum hydride, borane, and the like. The reaction is typically maintained at from 0° to 30° C. until it is substantially complete. Conventional workup of the reaction solution can be followed by isolation/purification processes such as crystallization, chromatography, high performance liquid chromatography (HPLC), and the like, to provide for compound 11′.

In the next step, the diol is converted to a suitable leaving group, at least a stoichiometric amount of tosyl chloride is added to compound 11′, in an inert diluent such as THF, MeCN, toluene and the like in the presence of a suitable base such as triethylamine, diisopropylethylamine, pyridine and the like. The reaction is typically maintained at from 0° to 30° C. until it is substantially complete. The Ts group is illustrative only and other conventional leaving groups such as iodo, bromo, triflate, mesylate and the like could be used. Conventional workup of the reaction solution can be followed by isolation/purification processes such as crystallization, chromatography, high performance liquid chromatography (HPLC), and the like to provide for compound 12′.

In the final step, at least a stoichiometric amount of compound 12′ is added to compound 7′, in an inert diluent such as THF, MeCN, toluene and the like in the presence of a suitable base such as triethylamine, diisopropylethylamine, pyridine and the like. The reaction is typically maintained at from 80° to 120° C. until it is substantially complete. Conventional workup of the reaction solution can be followed by isolation/purification processes such as crystallization, chromatography, high performance liquid chromatography (HPLC), and the like to provide for compounds of formula I-A′, formula I-A, formula I-B, and sub-formulae thereof.

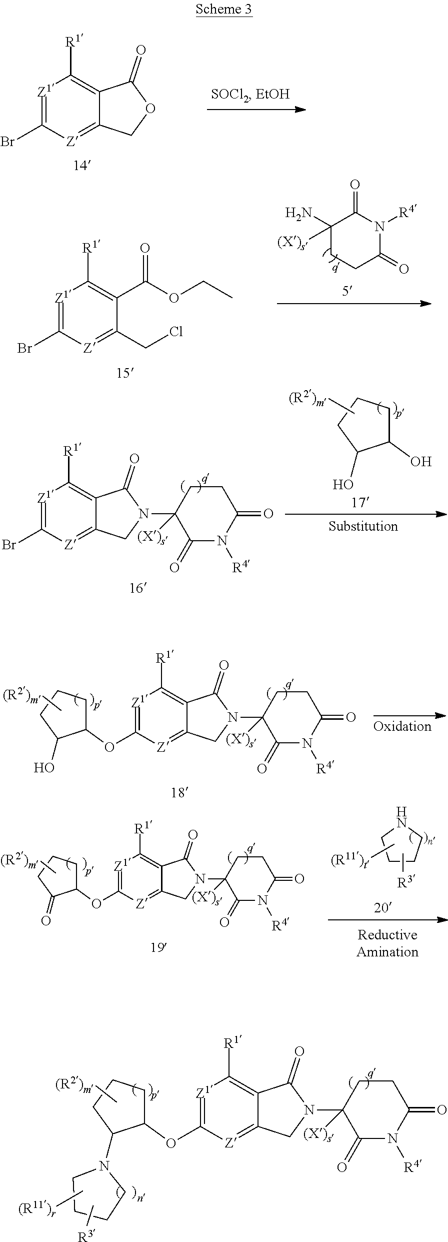

In some embodiments, compounds of formula I-A′, formula I-A, formula I-B, and sub-formulae thereof are prepared as shown in Scheme 3. In Scheme 3, the first step is a conventional esterification and chlorination reaction wherein at least a stoichiometric equivalent of thionyl chloride is combined with 5-bromoisobenzo-1(3H)-one, CAS #64169-34-2 (where R_{1′}═H; Z′ and Z^{1′} are each CH), compound 14′ in a diluent such as methanol, ethanol and the like. The reaction is typically maintained at from 50° to 80° C. until it is substantially complete. Conventional workup of the reaction solution can be followed by isolation/purification processes such as crystallization, chromatography, high performance liquid chromatography (HPLC), and the like to provide for compound 15′.

In the next step, at least a stoichiometric amount of 3-aminopiperidine-2,6-dione.hydrochloride, CAS #24666-56-6 (where R^{4′}═H; X′═H; q′=1; s′=1), compound 5′, is combined with compound 15′ in an inert diluent such as THF, DMF, MeCN, toluene and the like, typically in the presence of a suitable base such as triethylamine, diisopropylamine, DIEA, pyridine and the like. The reaction is typically maintained at from 80° to 100° C. until it is substantially complete. Conventional workup of the reaction solution can be followed by isolation/purification processes such as crystallization, chromatography, high performance liquid chromatography (HPLC), and the like to provide for compound 16′.

In the next step, at least a stoichiometric amount of compound 17′, is combined with compound 16′ in an inert diluent such as THF, MeCN, toluene and the like, typically in the presence of a suitable catalyst such as Ir, Cu(OAc)₂, SmI₂, and the like. The reaction is typically maintained at from 60° to 80° C. until it is substantially complete. Conventional workup of the reaction solution can be followed by isolation/purification processes such as crystallization, chromatography, high performance liquid chromatography (HPLC), and the like to provide for compound 18′.

In the next step, at least a stoichiometric amount of an oxidizing reagent is combined with compound 18′ under conventional oxidation reaction conditions well known in the art including the use of Jones Reagent, meta-chloroperoxybenzoic acid (mCPBA), Dess-Martin periodinane. The reaction is typically conducted in an inert solvent such as MeCN, THF, methylene chloride, toluene, and the like. The reaction is typically conducted at from about 0° to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 19′.

In the final step, at least a stoichiometric amount of a suitable amine, compound 20′ is combined with compound 19′ under conventional reductive amination reaction conditions well known in the art including the use of NaCNBH₃, NaBH(OAc)₃, NaBH₄ and the like. The reaction is typically conducted in an inert solvent such as MeCN, MeOH, THF, and the like. The reaction is typically conducted at from about 0° to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like, to provide compounds of formula I-A′, formula I-A, formula I-B, and sub-formulae thereof.

The general synthesis of the compounds described herein is further set forth in the reaction schemes below. In the schemes below, the variables are as defined throughout the specification. LG is a leaving group (including, but not limited to, Br, Cl, I, triflate, and the like).

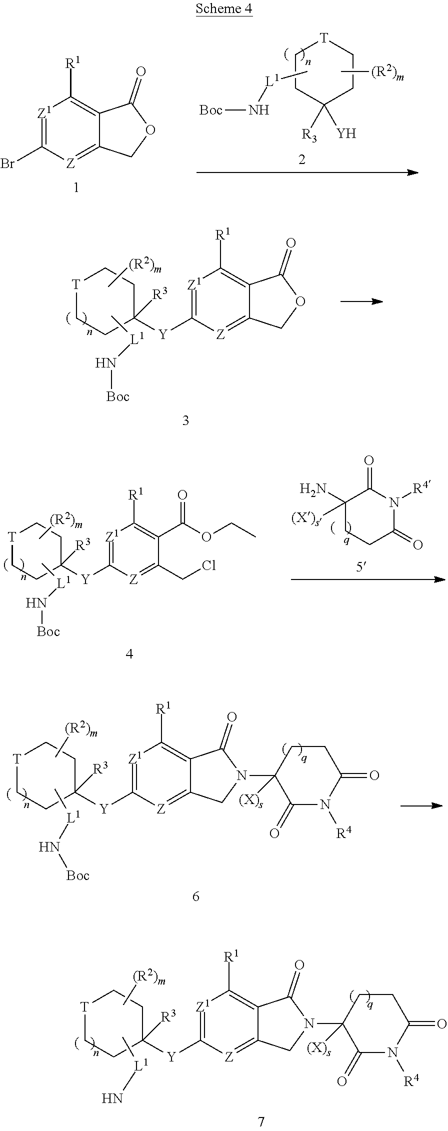

As to the reaction in Scheme 4, in the first step, at least a stoichiometric amount of protected compound 2 (Y is O or NR), is combined with compound 1, CAS #64169-34-2 (where R¹═H; Z and Z¹ are each CH), in an inert diluent such as THF, MeCN, toluene and the like, typically in the presence of a suitable catalyst such as Ir, Cu(OAc)₂, SmI₂, and the like. The reaction is typically maintained at from about 20° C. to about 50° C. until it is substantially complete. Conventional workup of the reaction solution can be followed by isolation/purification processes such as crystallization, chromatography, high performance liquid chromatography (HPLC), and the like to provide for compound 3.

In the next step, at least a stoichiometric equivalent of thionyl chloride is combined with compound 3 in a diluent such as methanol, ethanol and the like. The reaction is typically maintained at from about 50° C. to about 80° C. until it is substantially complete. Conventional workup of the reaction solution can be followed by isolation/purification processes such as crystallization, chromatography, high performance liquid chromatography (HPLC), and the like to provide for compound 4.

In the next step, at least a stoichiometric amount of compound 5, for example, 3-aminopiperidine-2,6-dione.hydrochloride, CAS #24666-56-6 (where R⁴═H; X═H; q=1; r=0; s=1), is combined with compound 4 in an inert diluent such as dichloromethane, tetrachloromethane and the like in the presence of a suitable base such as triethylamine, diisopropylethylamine, pyridine, and the like. The reaction is typically maintained at from about 0° C. to about 30° C. until it is substantially complete. Conventional workup of the reaction solution can be followed by isolation/purification processes such as crystallization, chromatography, high performance liquid chromatography (HPLC), and the like to provide for compound 6.

In the final step, the t-butoxycarbonyl (BOC) protecting group is removed by conventional conditions. The BOC group is illustrative only and other conventional amino blocking groups such as benzyl, 9-fluorenylmethoxycarbonyl (Fmoc), benzyloxycarbonyl (Cbz), p-nitrobenzyloxycarbonyl, and the like could be used. Upon reaction completion, conventional workup of the reaction solution can be followed by isolation/purification processes such as crystallization, chromatography, high performance liquid chromatography (HPLC), and the like to provide for compound 7, which serves as an intermediate for the synthesis of compounds of formula I-A, formula I-A′, formula I-B, and sub-formulae thereof.

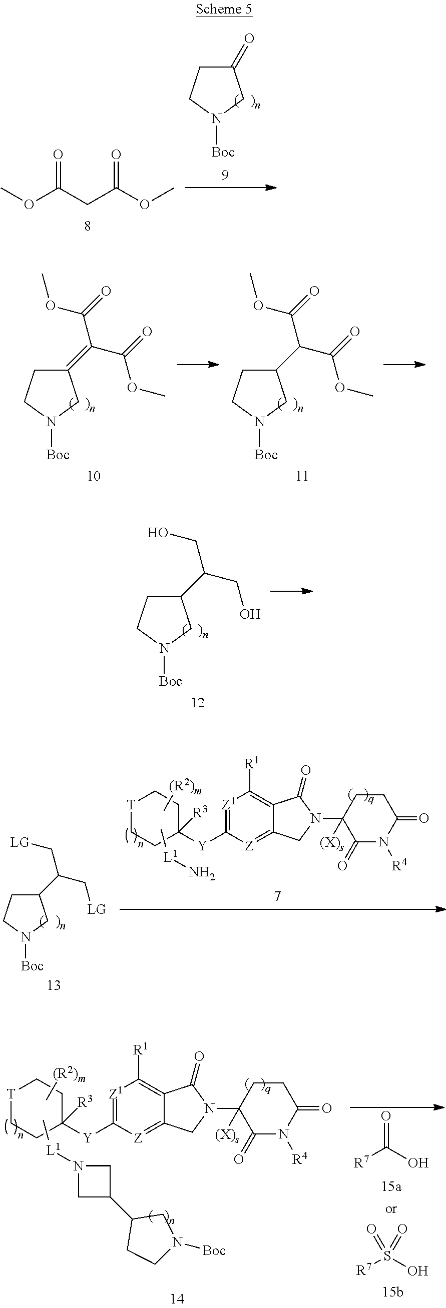

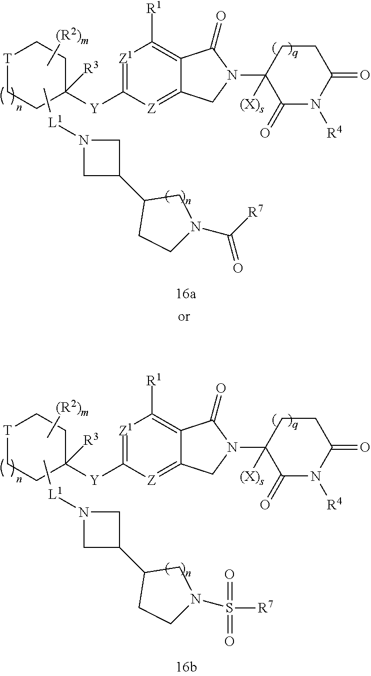

As to the reaction in Scheme 5, the first step is a Knoevenagel condensation reaction wherein at least a stoichiometric equivalent of a protected amino ketone 9 is combined with dimethylmalonate, compound 8, in an inert diluent such as DMF, DCM, MeCN and the like in the presence of a suitable base such as piperidine, pyridine, pyrrolidine and the like. The reaction is typically maintained at from 20° C. to 80° C. until it is substantially complete. Conventional workup of the reaction solution can be followed by isolation/purification processes such as crystallization, chromatography, high performance liquid chromatography (HPLC), and the like to provide for compound 10.

In the next step, compound 10 is reduced under conventional hydrogenation reaction conditions well known in the art, including the use palladium on carbon as catalyst under a hydrogen atmosphere (Organic Syntheses.; Collective Volume, 5, p. 30). Other reducing reagents are well known in the art. The reaction is typically conducted in an inert solvent such as EtOH, ethyl acetate, toluene, and the like. The reaction is typically conducted from about 20° C. to about 60° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 11.

In the next step, at least a stoichiometric amount of compound 11, in an inert diluent such as THF, MeCN, toluene and the like, is reacted with a suitable reducing reagent such as lithium aluminum hydride, borane, and the like. The reaction is typically maintained at from 0° C. to 30° C. until it is substantially complete. Conventional workup of the reaction solution can be followed by isolation/purification processes such as crystallization, chromatography, high performance liquid chromatography (HPLC), and the like to provide for compound 12.

In the next step, the diol is converted to a suitable leaving group, such as a tosyloxy group. At least a stoichiometric amount of tosyl chloride is added to compound 12, in an inert diluent such as THF, MeCN, toluene, and the like in the presence of a suitable base such as triethylamine, diisopropylethylamine, pyridine, and the like. The reaction is typically maintained at from about 0° C. to about 30° C. until it is substantially complete. The tosyl group is illustrative only and other conventional leaving groups such as iodo, bromo, triflate, mesylate and the like could be used. Conventional workup of the reaction solution can be followed by isolation/purification processes such as crystallization, chromatography, high performance liquid chromatography (HPLC), and the like to provide for compound 13.

In the next step, at least a stoichiometric amount of compound 13 is added to compound 7, in an inert diluent such as THF, MeCN, toluene and the like in the presence of a suitable base such as triethylamine, diisopropylethylamine, pyridine and the like. The reaction is typically maintained at from 80° C. to 120° C. until it is substantially complete. Conventional workup of the reaction solution can be followed by isolation/purification processes such as crystallization, chromatography, high performance liquid chromatography (HPLC), and the like to provide for compound 14.

In the next step, the t-butoxycarbonyl (t-BOC) protecting group is removed by conventional conditions to provide the cyclic amine. The t-BOC group is illustrative only and other conventional amino blocking groups such as benzyl, 9-fluorenylmethoxycarbonyl (Fmoc), benzyloxycarbonyl (Cbz), p-nitrobenzyloxycarbonyl and the like. Upon reaction completion, conventional workup of the reaction solution can be followed by isolation/purification processes such as crystallization, chromatography, high performance liquid chromatography (HPLC), and the like.

In the final step, at least a stoichiometric amount of a suitable carboxylic acid, compound 15a, is combined with the cyclic amine from the previous step, under conventional amidation reaction conditions well known in the art, including the use of N,N-dicyclohexylcarbodiimide (DCC) as an activation agent for the carboxyl group. Other activation agents are well known in the art. The reaction is typically conducted in an inert solvent such as chloroform, methylene chloride, toluene, N,N-dimethylformamide, and the like. The reaction is typically conducted at from about 0° C. to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 16a.

Similarly, the final step can be completed by reacting at least a stoichiometric amount of a suitable sulfonyl chloride, compound 15b, with the cyclic amine from the penultimate step, under conventional reaction conditions well known in the art, including the use of a base, including but not limited to sodium hydroxide, pyridine, triethylamine, and the like. The reaction is typically conducted at from about 25° C. to about 50° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 16b.

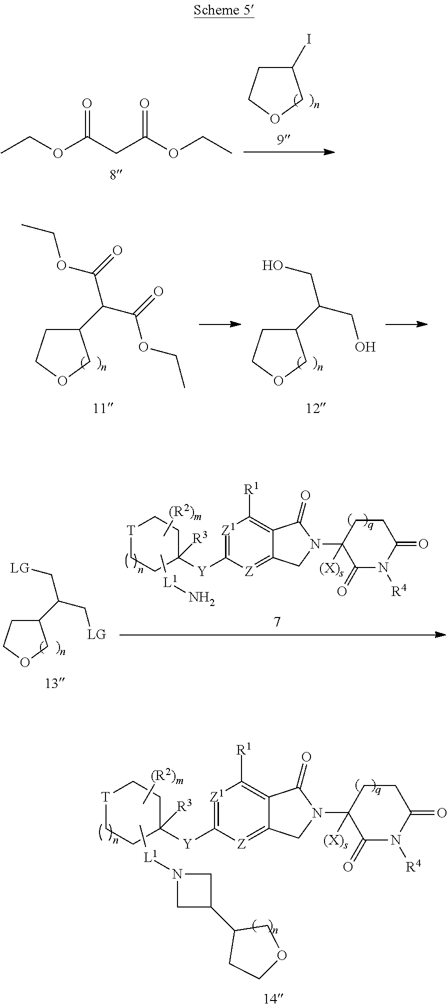

Similarly, in some embodiments, compounds of formula I-A, formula I-A′, and sub-formulae thereof are prepared as shown in Scheme 5′. In the first step, at least a stoichiometric equivalent of an iodo cyclic ether 9″ is combined with diethylmalonate, compound 8″, in the corresponding alcohol and in the presence of a suitable base, such as sodium ethoxide or the like. The reaction is typically maintained at from 50° C. to 80° C. until it is substantially complete. Conventional workup of the reaction solution can be followed by isolation/purification processes such as crystallization, chromatography, high performance liquid chromatography (HPLC), and the like to provide for compound 11″.

In the next step, at least a stoichiometric amount of compound 11″, in an inert diluent such as THF, MeCN, toluene and the like, is reacted with a suitable reducing reagent such as lithium aluminum hydride, borane, and the like. The reaction is typically maintained at from 0° C. to 30° C. until it is substantially complete. Conventional workup of the reaction solution can be followed by isolation/purification processes such as crystallization, chromatography, high performance liquid chromatography (HPLC), and the like to provide for compound 12″.

In the next step, the diol is converted to a suitable leaving group, such as a tosyloxy group. At least a stoichiometric amount of tosyl chloride is added to compound 12″, in an inert diluent such as THF, MeCN, toluene, and the like in the presence of a suitable base such as triethylamine, diisopropylethylamine, pyridine, and the like. The reaction is typically maintained at from about 0° C. to about 30° C. until it is substantially complete. The tosyl group is illustrative only and other conventional leaving groups such as iodo, bromo, triflate, mesylate and the like could be used. Conventional workup of the reaction solution can be followed by isolation/purification processes such as crystallization, chromatography, high performance liquid chromatography (HPLC), and the like to provide for compound 13″.

In the final step, at least a stoichiometric amount of compound 13″ is added to compound 7, in an inert diluent such as THF, MeCN, toluene and the like in the presence of a suitable base such as triethylamine, diisopropylethylamine, pyridine and the like. The reaction is typically maintained at from 80° C. to 120° C. until it is substantially complete. Conventional workup of the reaction solution can be followed by isolation/purification processes such as crystallization, chromatography, high performance liquid chromatography (HPLC), and the like to provide for compound 14″.

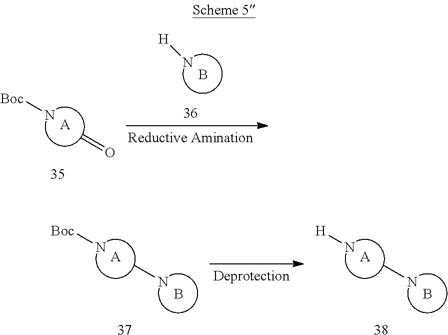

As to the reaction in Scheme 5″, in the first step, at least a stoichiometric amount of a suitable amine, compound 36, is combined with compound 35 under conventional reductive amination reaction conditions well known in the art, including the use of NaCNBH₃, NaBH(OAc)₃, NaBH₄ and the like. The reaction is typically conducted in an inert solvent, such as MeCN, MeOH, THF, and the like. The reaction is typically conducted at from about 0° C. to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like, to provide compound 37.

In the final step, the t-butoxycarbonyl (t-BOC) protecting group is removed by conventional conditions to provide the cyclic amine 38. The t-BOC group is illustrative only, and other conventional amino blocking groups such as benzyl, 9-fluorenylmethoxycarbonyl (Fmoc), benzyloxycarbonyl (Cbz), p-nitrobenzyloxycarbonyl, and the like may be employed using methods well known in the art. Upon reaction completion, conventional workup of the reaction solution can be followed by isolation/purification processes such as crystallization, chromatography, high performance liquid chromatography (HPLC), and the like.

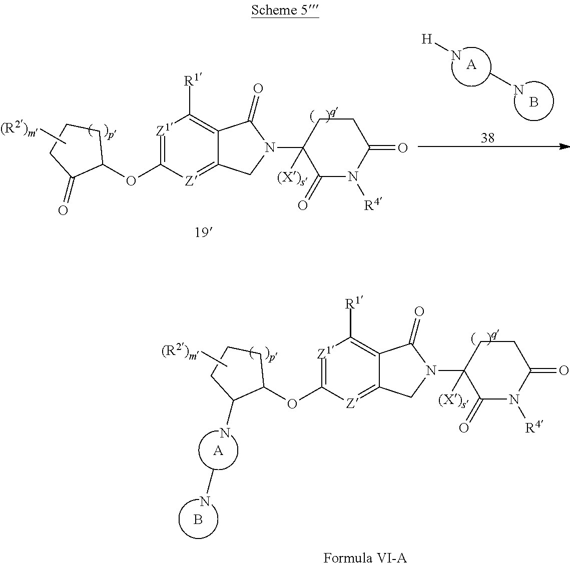

In some embodiments, compounds of formula VI-A, and sub-formulae thereof, are prepared as shown in Scheme 5′″. As to the reaction in Scheme 5′″, compound 38 is prepared as shown in scheme 5″ above. At least a stoichiometric amount of a suitable amine, compound 38, is combined with compound 19′ under conventional reductive amination reaction conditions well known in the art, including the use of NaCNBH₃, NaBH(OAc)₃, NaBH₄ and the like. The reaction is typically conducted in an inert solvent such as MeCN, MeOH, THF, and the like. The reaction is typically conducted at from about 0° C. to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like, to provide compounds of formula VI-A.

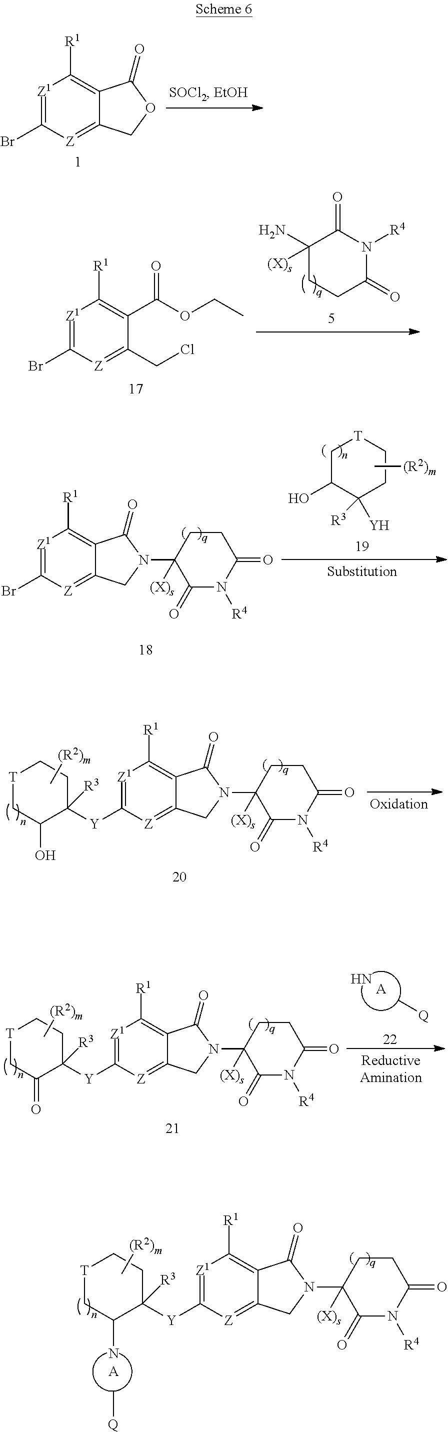

In some embodiments, compounds of formula I-A, formula I-A′, formula I-B, and sub-formulae thereof are prepared as shown in Scheme 6. In Scheme 6, the first step is a conventional esterification and chlorination reaction wherein at least a stoichiometric equivalent of thionyl chloride is combined with 5-bromoisobenzo-1(3H)-one, CAS #64169-34-2 (where R¹═H; Z and Z¹ are each CH), compound 1, in a diluent such as methanol, ethanol and the like. The reaction is typically maintained at from 50° C. to 80° C. until it is substantially complete. Conventional workup of the reaction solution can be followed by isolation/purification processes such as crystallization, chromatography, high performance liquid chromatography (HPLC), and the like to provide for compound 17.

In the next step, at least a stoichiometric amount of 3-aminopiperidine-2,6-dione.hydrochloride, CAS #24666-56-6 (where R⁴═H; X═H; q=1; r=0; s=1), compound 5, is combined with compound 17 in an inert diluent such as THF, DMF, MeCN, toluene, and the like, typically in the presence of a suitable base such as triethylamine, diisopropylamine, DIEA, pyridine, and the like. The reaction is typically maintained at from about 80° C. to about 100° C. until it is substantially complete. Conventional workup of the reaction solution can be followed by isolation/purification processes such as crystallization, chromatography, high performance liquid chromatography (HPLC), and the like to provide for compound 18.

In the next step, at least a stoichiometric amount of compound 19, is combined with compound 18 in an inert diluent such as THF, MeCN, toluene and the like, typically in the presence of a suitable catalyst such as Ir, Cu(OAc)₂, SmI₂, and the like. The reaction is typically maintained at from about 60° C. to about 80° C. until it is substantially complete. Conventional workup of the reaction solution can be followed by isolation/purification processes such as crystallization, chromatography, high performance liquid chromatography (HPLC), and the like to provide for compound 20.

In the next step, at least a stoichiometric amount of an oxidizing reagent is combined with compound 20 under conventional oxidation reaction conditions well known in the art including the use of Jones Reagent, meta-chloroperoxybenzoic acid (mCPBA), or Dess-Martin periodinane. The reaction is typically conducted in an inert solvent such as MeCN, THF, methylene chloride, toluene, and the like. The reaction is typically conducted at from about 0° C. to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 21.

In the final step, at least a stoichiometric amount of a suitable amine, compound 22, is combined with compound 21 under conventional reductive amination reaction conditions well known in the art including the use of NaCNBH₃, NaBH(OAc)₃, NaBH₄ and the like. The reaction is typically conducted in an inert solvent such as MeCN, MeOH, THF, and the like. The reaction is typically conducted at from about 0° C. to about 30° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like, to provide compounds of formula I-A, formula I-A′, formula I-B, and sub-formulae thereof.

The general synthesis of fluorinated compounds of formula I-A and sub-formulae thereof is described herein as set forth in the schemes 7-9 below. In the Schemes below, the variables are as defined throughout the specification. LG is a leaving group (including, but not limited to, Br, Cl, I, triflate, and the like).

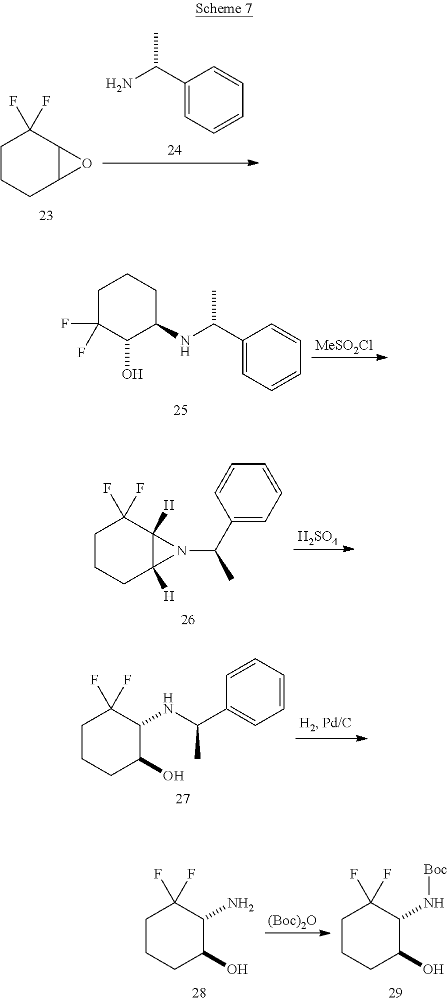

As to the reaction in Scheme 7, in the first step, at least a stoichiometric amount of (R)-1-phenylethan-1-amine, compound 24 is combined with compound 23, CAS #1109284-38-9 in an inert diluent such as dichloromethane, dichloroethane, and the like typically in the presence of a suitable catalyst such as trimethyl aluminum. The reaction is typically maintained at from about 0° C. to about 20° C. until it is substantially complete. Conventional workup of the reaction solution can be followed by isolation/purification processes such as crystallization, chromatography, high performance liquid chromatography (HPLC), and the like to provide for compound 25.

In the next step, at least a stoichiometric equivalent of methanesulfonyl chloride is combined with compound 25 in a diluent such as tetrahydrofuran, dioxane, DMF and the like, typically in the presence of a suitable base such as pyridine, triethylamine and the like. The reaction is typically maintained at from about 20° C. to about 100° C. until it is substantially complete. Conventional workup of the reaction solution can be followed by isolation/purification processes such as crystallization, chromatography, high performance liquid chromatography (HPLC), and the like to provide for compound 26.

In the next step, hydrolysis of compound 26 [WO2009/14620] under acidic conditions well known in the art, including the use sulfuric acid, trifluoroacetic acid and the like. The reaction is typically conducted in water. The reaction is typically conducted at from about 80° C. to about 100° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 27.

In the next step, hydrogenolysis of the of (R)-1-phenylethyl group of compound 27 [Org. Lett., 2003, 5, 761-764] under conventional hydrogenation reaction conditions well known in the art, including the use palladium on carbon as catalyst under a hydrogen atmosphere. The reaction is typically conducted in an inert solvent such as EtOH, ethyl acetate, toluene, and the like. The reaction is typically conducted at from about 20° C. to about 60° C. for a period of time sufficient for substantial completion of the reaction as evidenced by e.g., thin layer chromatography. Upon reaction completion, conventional workup of the reaction solution can be followed by isolation/purification processes such as crystallization, chromatography, high performance liquid chromatography (HPLC), and the like to provide for compound 28.

In the final step, the amine 28 is protected with t-butoxycarbonyl (BOC) group with. At least a stoichiometric amount of a BOC anhydride is combined with compound 28 in an inert diluent such as dichloromethane, dichloroethane, THF, and the like. The reaction is typically maintained at from about 20° C. to about 50° C. until it is substantially complete. Conventional workup of the reaction solution can be followed by isolation/purification processes such as crystallization, chromatography, high performance liquid chromatography (HPLC), and the like to provide for compound 29. The BOC group is illustrative only and other conventional amino blocking groups such as benzyl, 9-fluorenylmethoxycarbonyl (Fmoc), benzyloxycarbonyl (Cbz), p-nitrobenzyloxycarbonyl and the like could be used.

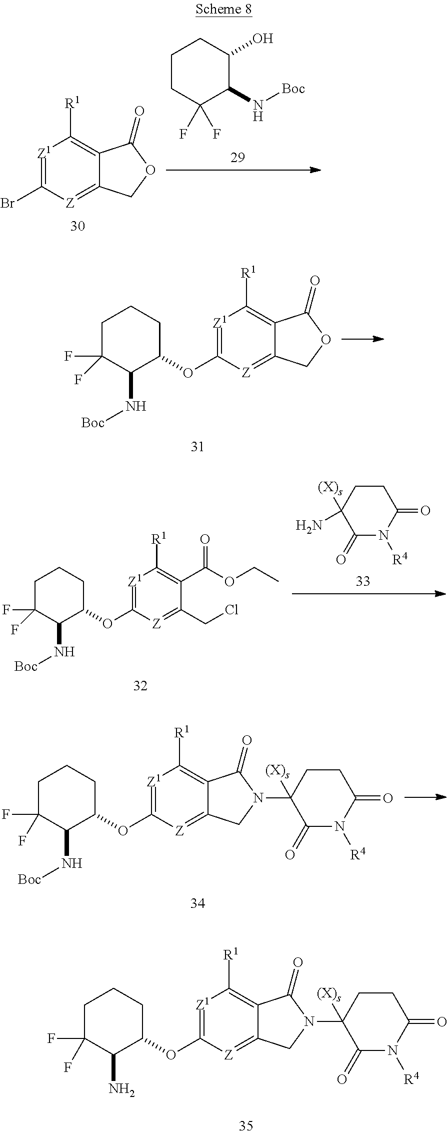

As to the reaction in Scheme 8, in the first step, at least a stoichiometric amount of protected amino alcohol, compound 29, is combined with compound 30, CAS #64169-34-2 (where R¹═H; Z and Z¹ are each CH), in an inert diluent such as THF, MeCN, toluene, and the like, typically in the presence of a suitable catalyst such as Ir, Cu(OAc)₂, SmI₂, and the like. The reaction is typically maintained at from about 20° C. to about 50° C. until it is substantially complete. Conventional workup of the reaction solution can be followed by isolation/purification processes such as crystallization, chromatography, high performance liquid chromatography (HPLC), and the like to provide for compound 31.

In the next step, at least a stoichiometric equivalent of thionyl chloride is combined with compound 31 in a diluent such as methanol, ethanol and the like. The reaction is typically maintained at from about 50° C. to about 80° C. until it is substantially complete. Conventional workup of the reaction solution can be followed by isolation/purification processes such as crystallization, chromatography, high performance liquid chromatography (HPLC), and the like to provide for compound 32.

In the next step, at least a stoichiometric amount of 3-aminopiperidine-2,6-dione.hydrochloride, CAS #24666-56-6 (where R⁴═H; X=H), compound 33, is combined with compound 32 in an inert diluent such as dichloromethane, tetrachloromethane and the like in the presence of a suitable base such as triethylamine, diisopropylethylamine, pyridine and the like. The reaction is typically maintained at from 0° C. to 30° C. until it is substantially complete. Conventional workup of the reaction solution can be followed by isolation/purification processes such as crystallization, chromatography, high performance liquid chromatography (HPLC), and the like to provide for compound 34.

In the final step, the t-butoxycarbonyl (BOC) protecting group is removed by conventional conditions. The BOC group is illustrative only and other conventional amino blocking groups such as benzyl, 9-fluorenylmethoxycarbonyl (Fmoc), benzyloxycarbonyl (Cbz), p-nitrobenzyloxycarbonyl and the like could be used. Upon reaction completion, conventional workup of the reaction solution can be followed by isolation/purification processes such as crystallization, chromatography, high performance liquid chromatography (HPLC), and the like to provide for compound 35, which serves as an intermediate for the synthesis of compounds of formula I-A and sub-formulae thereof.

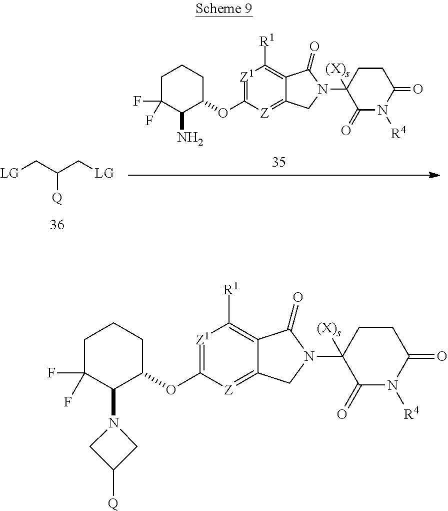

In some embodiments, compounds of formula I-A and sub-formulae thereof are prepared as shown in Scheme 9. As to the reaction in Scheme 9, compound 36 is prepared as shown in scheme 5 above, wherein compound 36 is compound 13, when Q=cyclic protected amine. In the next step, at least a stoichiometric amount of compound 36 is added to compound 35, in an inert diluent such as THF, MeCN, toluene, and the like in the presence of a suitable base such as triethylamine, diisopropylethylamine, pyridine, and the like. The reaction is typically maintained at from about 80° C. to about 120° C. until it is substantially complete. Conventional workup of the reaction solution can be followed by isolation/purification processes such as crystallization, chromatography, high performance liquid chromatography (HPLC), and the like to provide for fluorinated compounds of formula I-A.

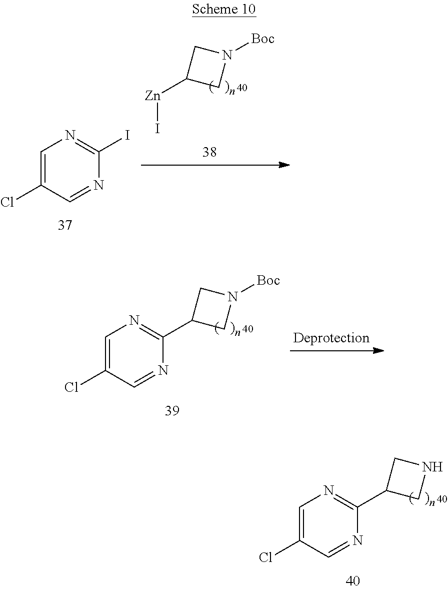

As to reaction of Scheme 10, the first step is a conventional Negishi coupling reaction wherein at least a stoichiometric equivalent of an alkyl zinc reagent, compound 38 is combined with a heteroaryl halide, compound 37, in a diluent such as THF, MeCN, and the like. The reaction is typically maintained at from 0° C. to 50° C. until it is substantially complete. Conventional workup of the reaction solution can be followed by isolation/purification processes such as crystallization, chromatography, high performance liquid chromatography (HPLC), and the like to provide for compound 39.

In the final step, the t-butoxycarbonyl (BOC) protecting group is removed by conventional conditions to provide for compound 40. The BOC group is illustrative only and other conventional amino blocking groups such as benzyl, 9-fluorenylmethoxycarbonyl (Fmoc), benzyloxycarbonyl (Cbz), p-nitrobenzyloxycarbonyl and the like can be used. Upon reaction completion, conventional workup of the reaction solution can be followed by isolation/purification processes such as crystallization, chromatography, high performance liquid chromatography (HPLC), and the like to provide for compound 40. Compound 40 can be used in the reductive amination step shown in Scheme 3, to provide compounds of formula I-B.

Other starting materials used herein are either well known in the art, commercially available, or can be prepared by conventional synthetic methods.

### Methods

In one embodiment, the compounds of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, and VI-B, and compositions described herein are useful in methods for modulating cereblon activity. The methods comprise administering to a subject an effective amount of a compound, or a pharmaceutically acceptable salt, solvate, stereoisomer, and/or tautomer thereof or a pharmaceutical composition comprising said compound, or a pharmaceutically acceptable salt, solvate, stereoisomer, and/or tautomer thereof as described herein.

In one embodiment, the compounds of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, and VI-B, and compositions described herein are useful in methods for treating a IKZF2 dependent disease or disorder or a disease or disorder that is mediated, at least in part by, IKZF2. The methods comprise administering to a subject suffering from a IKZF2 dependent disease or disorder an effective amount of a compound, or a pharmaceutically acceptable salt, solvate, stereoisomer, and/or tautomer thereof or a pharmaceutical composition comprising said compound, or a pharmaceutically acceptable salt, solvate, stereoisomer, and/or tautomer thereof as described herein.

In one embodiment, the compounds of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, and/or VI-B, and compositions described herein selectively modulate IKZF (e.g., over GSPT1). In some embodiments, the compounds of formula I-A, II-A, IIA-A, IIB-A, IIC-A, III-A, IIIA-A, IIIB-A, IIIC-A, IIID-A, IV-A, IVA-A, IVB-A, V-A, VA-A, VB-A, VI-A, VIA-A, VIB-A, VII-A, VIIA-A, VIIB-A, VIII-A, VIIIA-A, VIIIB-A, IX-A, IXA-A, IXB-A, I-A′, II-A′, III-A′, IV-A′, V-A′, VI-A′, I-B, II-B, III-B, IV-B, V-B, and/or VI-B, and compositions described herein selectively modulate IKZF2 over GSPT1.

In one embodiment, there is provided a compound, or a pharmaceutically acceptable salt, solvate, stereoisomer, and/or tautomer thereof or a pharmaceutical composition comprising said compound, or a pharmaceutically acceptable salt, solvate, stereoisomer, and/or tautomer thereof as described herein for use in treating an IKZF2 dependent disease or disorder.

In one embodiment, the method relates to a compound, or a pharmaceutically acceptable salt, solvate, stereoisomer, and/or tautomer thereof or a pharmaceutical composition comprising said compound, or a pharmaceutically acceptable salt, solvate, stereoisomer, and/or tautomer thereof as described herein for use in manufacture of a medicament for reducing IKZF2 protein levels where reduction of such protein levels treats or ameliorates the diseases or disorder.

In one embodiment, the methods described herein comprise use of a prodrug of the compounds described herein.

In one embodiment, the method relates to a compound, or a pharmaceutically acceptable salt, solvate, stereoisomer, and/or tautomer thereof or a pharmaceutical composition comprising said compound, or a pharmaceutically acceptable salt, solvate, stereoisomer, and/or tautomer thereof as described herein for use as described herein, wherein the concentration of compound required for cereblon target engagement dose response IC₅₀ is in the range of about 0.003 μM to about 0.06 μM. The cereblon target engagement dose response IC₅₀ is measured by the assay described in the biological example. In some embodiments, the cereblon binding concentration is from about 0.003 μM to about 0.006 μM, from about 0.005 μM to about 0.008 μM, from about 0.007 μM to about 0.01 μM, from about 0.009 μM to about 0.012 μM, from about 0.012 μM to about 0.015 μM, from about 0.015 μM to about 0.018 μM, from about 0.018 μM to about 0.021 μM, from about 0.021 μM to about 0.024 μM, from about 0.024 μM to about 0.027 μM, or from about 0.027 μM to about 0.030 PM. In some embodiments, the cereblon binding concentration is less than 0.015 μM. In some embodiments, the cereblon binding concentration is less than 0.010 μM. In some embodiments, the cereblon binding concentration is less than 0.005 μM.

In one embodiment, the method relates to a compound, or a pharmaceutically acceptable salt, solvate, stereoisomer, and/or tautomer thereof or a pharmaceutical composition comprising said compound, or a pharmaceutically acceptable salt, solvate, stereoisomer, and/or tautomer thereof as described herein for use as described herein, wherein the IKZF2 degradation at 1 μM concentration of the compounds described herein is in the range of about 25%-99%. The IKZF2 degradation is measured by the assay described in the biological example. In some embodiments, the IKZF2 degradation is from about 25% to about 50%, from about 45% to about 70%, from about 65% to about 90% or from about 75% to about 99%. In some embodiments, the IKZF2 degradation is from about 25% to about 35%, from about 35% to about 45%, from about 45% to about 55%, from about 55% to about 65%, from about 65% to about 75%, from about 75% to about 85%, from about 85% to about 99%. In some embodiments, the IKZF2 degradation is more than 60%. In some embodiments, the IKZF2 degradation is more than 70%. In some embodiments, the IKZF2 degradation is more than 80%. In some embodiments, the IKZF2 degradation is more than 90%.

Non limiting examples of IKZF2 dependent diseases or disorders include proliferative diseases or disorders which may be non-cancerous or cancerous.

Examples of non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcoidosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

In certain embodiments, the compounds or compositions described herein are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer. In certain embodiments, compounds or compositions described herein are active against solid tumors.

In certain embodiments, the compounds or compositions described herein are useful for the treatment of cancer (including, but not limited to, glioblastoma, retinoblastoma, breast cancer, cervical cancer, colon and rectal cancer, leukemia, lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer).

In some embodiments, examples of cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstrom macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), Merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilms' Tumor.

In certain embodiments, the compounds described herein are useful for the treatment of cancer (including, but not limited to, glioblastoma, retinoblastoma, breast cancer, cervical cancer, colon and rectal cancer, leukemia, lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer) and/or any other cancer described herein.

In certain embodiments, the compounds described herein are useful in the treatment of cancers and other proliferative disorders, including, but not limited to breast cancer, cervical cancer, colon and rectal cancer, leukemia, lung cancer, melanoma, multiple myeloma, non-Hodgkin's lymphoma, ovarian cancer, pancreatic cancer, prostate cancer, and gastric cancer. In certain embodiments, the compounds are active against solid tumors.

In certain embodiments, the compounds and compositions described herein are useful in treating IKZF2 dependent diseases or disorders such as liposarcoma, glioblastoma, bladder cancer, adrenocortical cancer, multiple myeloma, colorectal cancer, non-small cell lung cancer, Human Papilloma Virus-associated cervical, oropharyngeal, penis, anal, thyroid, or vaginal cancer or Epstein-Barr Virus-associated nasopharyngeal carcinoma, gastric cancer, rectal cancer, thyroid cancer, Hodgkin lymphoma or diffuse large B-cell lymphoma. The cancer may be selected from prostate cancer, breast carcinoma, lymphomas, leukemia, myeloma, bladder carcinoma, colon cancer, cutaneous melanoma, hepatocellular carcinoma, endometrial cancer, ovarian cancer, cervical cancer, lung cancer, renal cancer, glioblastoma multiform, glioma, thyroid cancer, parathyroid tumor, nasopharyngeal cancer, tongue cancer, pancreatic cancer, esophageal cancer, cholangiocarcinoma, gastric cancer, soft tissue sarcomas, rhabdomyosarcoma (RMS), synovial sarcoma, osteosarcoma, rhabdoid cancers, cancer for which the immune response is deficient, an immunogenic cancer, and Ewing's sarcoma. In one embodiment, the IKZF2-dependent disease or disorder is a disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST). In another embodiment, the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST). In another embodiment, the IKZF2-dependent disease or disorder is a disease or disorder is selected from non-small cell lung cancer (NSCLC), melanoma, triple-negative breast cancer (TNBC), nasopharyngeal cancer (NPC), and microsatellite stable colorectal cancer (mssCRC).

The compounds of the disclosure can be administered in effective amounts to treat or prevent a disorder and/or prevent the development thereof in subjects.

In general, methods of using the compounds of the present application comprise administering to a subject in need thereof a therapeutically effective amount of a compound as described herein.

In certain embodiments, compounds as described herein are useful in the treatment of proliferative diseases (e.g., cancer, benign neoplasms, inflammatory disease, and autoimmune diseases). In certain embodiments, according to the methods of treatment of the present application, levels of cell proteins of interest, e.g., pathogenic and oncogenic proteins are modulated, or their growth is inhibited or the proteins are degraded by contacting said cells with an compound or composition, as described herein. In other embodiments, the compounds are useful in treating cancer.

Thus, in another aspect of the application, methods for the treatment of cancer are provided comprising administering a therapeutically effective amount of compound or composition, as described herein, to a subject in need thereof. In certain embodiments, a method for the treatment of cancer is provided comprising administering a therapeutically effective amount of a compound, or a pharmaceutical composition comprising a compound as described herein to a subject in need thereof, in such amounts and for such time as is necessary to achieve the desired result. In some embodiments, the compounds of present application are administered orally or intravenously. In certain embodiments of the present application a “therapeutically effective amount” of the compound or pharmaceutical composition is that amount effective for killing or inhibiting the growth of tumor cells. The compounds and compositions, according to the method of the present application, may be administered using any amount and any route of administration effective for killing or inhibiting the growth of tumor cells. Thus, the expression “amount effective to kill or inhibit the growth of tumor cells,” as used herein, refers to a sufficient amount of agent to kill or inhibit the growth of tumor cells. The exact amount required will vary from subject to subject, depending on the species, age, and general condition of the subject, the severity of the disease, the particular anticancer agent, its mode of administration, and the like. In certain embodiments of the present application a “therapeutically effective amount” of the-compound or pharmaceutical composition described herein is that amount effective for reducing the levels of target proteins. In certain embodiments of the present application a “therapeutically effective amount” of the compound or pharmaceutical composition is that amount effective to kill or inhibit the growth of skin cells.

In certain embodiments, the method involves the administration of a therapeutically effective amount of the compound or a pharmaceutically acceptable derivative thereof to a subject (including, but not limited to a human or other mammal in need of it.

Additionally, the present application provides pharmaceutically acceptable derivatives of the compounds, and methods of treating a subject using these compounds, pharmaceutical compositions thereof, or either of these in combination with one or more additional therapeutic agents.

Another aspect of the application relates to a method of treating or lessening the severity of a disease or condition associated with a proliferation disorder in a patient, said method comprising a step of administering to said patient, a compound of Formula I-A, Formula I-A′, Formula I-B, or sub-formulae thereof, or a composition comprising a compound of Formula I-A, Formula I-A′, Formula I-B, or sub-formulae thereof.

It will be appreciated that the compounds and compositions, according to the method of the present application, may be administered using any amount and any route of administration effective for the treatment of cancer and/or disorders associated with cell hyperproliferation. For example, when using the compounds for the treatment of cancer, the expression “effective amount” as used herein, refers to a sufficient amount of agent to inhibit cell proliferation, or refers to a sufficient amount to reduce the effects of cancer. The exact amount required will vary from subject to subject, depending on the species, age, and general condition of the subject, the severity of the diseases, the particular anticancer agent, its mode of administration, and the like.

The present application provides methods for the treatment of a proliferative disorder in a subject in need thereof by administering to a subject in need of such treatment, a therapeutically effective amount of a compound of the present application, or a pharmaceutically acceptable salt, solvate, stereoisomer, and/or tautomer thereof. The proliferative disorder can be cancer or a precancerous condition. The present application further provides the use of a compound of the present application, or a pharmaceutically acceptable salt, salt, solvate, stereoisomer, and/or tautomer thereof, for the preparation of a medicament useful for the treatment of a proliferative disorder.

The present application also provides methods of protecting against a proliferative disorder in a subject in need thereof by administering a therapeutically effective amount of compound of the present application, or a pharmaceutically acceptable salt, salt, solvate, stereoisomer, and/or tautomer thereof, to a subject in need of such treatment. The proliferative disorder can be cancer or a precancerous condition. The present application also provides the use of compound of the present application, or a pharmaceutically acceptable salt, salt, solvate, stereoisomer, and/or tautomer thereof, for the preparation of a medicament useful for the prevention of a proliferative disorder.

As used herein, the term “proliferative disorder” refers to conditions in which unregulated or abnormal growth, or both, of cells can lead to the development of an unwanted condition or disease, which may or may not be cancerous. Exemplary proliferative disorders of the application encompass a variety of conditions wherein cell division is deregulated. Exemplary proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A proliferative disorder includes a precancer or a precancerous condition. A proliferative disorder includes cancer. Preferably, the methods provided herein are used to treat or alleviate a symptom of cancer. The term “cancer” includes solid tumors, as well as hematologic tumors and/or malignancies. A “precancer cell” or “precancerous cell” is a cell manifesting a proliferative disorder that is a precancer or a precancerous condition. A “cancer cell” or “cancerous cell” is a cell manifesting a proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

A “proliferative disorder of the hematologic system” is a proliferative disorder involving cells of the hematologic system. A proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A proliferative disorder of the hematologic system can include hyperplasia, dysplasia, and metaplasia of cells of the hematologic system. Preferably, compositions of the present application may be used to treat a cancer selected from the group consisting of a hematologic cancer of the present application or a hematologic proliferative disorder of the present application. A hematologic cancer of the present application can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “proliferative disorder of the lung” is a proliferative disorder involving cells of the lung. Proliferative disorders of the lung can include all forms of proliferative disorders affecting lung cells. Proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions of the present application may be used to treat lung cancer or proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructural heterogeneity (e.g., mixed cell types).

Proliferative disorders of the lung can include all forms of proliferative disorders affecting lung cells. Proliferative disorders of the lung can include lung cancer, precancerous conditions of the lung. Proliferative disorders of the lung can include hyperplasia, metaplasia, and dysplasia of the lung. Proliferative disorders of the lung can include asbestos-induced hyperplasia, squamous metaplasia, and benign reactive mesothelial metaplasia. Proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing proliferative disorders of the lung. Prior lung diseases that may predispose individuals to development of proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “proliferative disorder of the colon” is a proliferative disorder involving cells of the colon. Preferably, the proliferative disorder of the colon is colon cancer. Preferably, compositions of the present application may be used to treat colon cancer or proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Proliferative disorders of the colon can include all forms of proliferative disorders affecting colon cells. Proliferative disorders of the colon can include colon cancer, precancerous conditions of the colon, adenomatous polyps of the colon and metachronous lesions of the colon. A proliferative disorder of the colon can include adenoma. Proliferative disorders of the colon can be characterized by hyperplasia, metaplasia, and dysplasia of the colon. Prior colon diseases that may predispose individuals to development of proliferative disorders of the colon can include prior colon cancer. Current disease that may predispose individuals to development of proliferative disorders of the colon can include Crohn's disease and ulcerative colitis. A proliferative disorder of the colon can be associated with a mutation in a gene selected from the group consisting of p53, ras, FAP and DCC. An individual can have an elevated risk of developing a proliferative disorder of the colon due to the presence of a mutation in a gene selected from the group consisting of p53, ras, FAP and DCC.

A “proliferative disorder of the pancreas” is a proliferative disorder involving cells of the pancreas. Proliferative disorders of the pancreas can include all forms of proliferative disorders affecting pancreatic cells. Proliferative disorders of the pancreas can include pancreas cancer, a precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “proliferative disorder of the prostate” is a proliferative disorder involving cells of the prostate. Proliferative disorders of the prostate can include all forms of proliferative disorders affecting prostate cells. Proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Proliferative disorders of the prostate can include hyperplasia, metaplasia, and dysplasia of the prostate.

A “proliferative disorder of the skin” is a proliferative disorder involving cells of the skin. Proliferative disorders of the skin can include all forms of proliferative disorders affecting skin cells. Proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Proliferative disorders of the skin can include hyperplasia, metaplasia, and dysplasia of the skin.

A “proliferative disorder of the ovary” is a proliferative disorder involving cells of the ovary. Proliferative disorders of the ovary can include all forms of proliferative disorders affecting cells of the ovary. Proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Proliferative disorders of the skin can include hyperplasia, metaplasia, and dysplasia of cells of the ovary.

A “proliferative disorder of the breast” is a proliferative disorder involving cells of the breast. Proliferative disorders of the breast can include all forms of proliferative disorders affecting breast cells. Proliferative disorders of the breast can include breast cancer, a precancer or precancerous condition of the breast, benign growths or lesions of the breast, and malignant growths or lesions of the breast, and metastatic lesions in tissue and organs in the body other than the breast. Proliferative disorders of the breast can include hyperplasia, metaplasia, and dysplasia of the breast.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “proliferative disorder”. A normal cell lacks unregulated or abnormal growth, or both, that can lead to the development of an unwanted condition or disease. Preferably, a normal cell possesses normally functioning cell cycle checkpoint control mechanisms.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Erma et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the application.

In certain embodiments, compounds of the application are useful in the treatment of proliferative diseases (e.g., cancer, benign neoplasms, inflammatory disease, and autoimmune diseases). In certain embodiments, according to the methods of treatment of the present application, levels of cell proteins of interest, e.g., pathogenic and oncogenic proteins are modulated, or their growth is inhibited by contacting said cells with an compound or composition, as described herein. In other embodiments, the compounds are useful in treating cancer.

In certain embodiments, the method involves the administration of a therapeutically effective amount of the compound or a pharmaceutically acceptable derivative thereof to a subject (including, but not limited to a human or animal) in need of it.

Additionally, the present application provides pharmaceutically acceptable derivatives of the compounds, and methods of treating a subject using these compounds, pharmaceutical compositions thereof, or either of these in combination with one or more additional therapeutic agents.

For example, other therapies or anticancer agents that may be used in combination with the compounds disclosed herein including surgery, radiotherapy, endocrine therapy, biologic response modifiers (interferons, interleukins, and tumor necrosis factor (TNF), to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabine, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of overview of cancer therapy see The Merck Manual, Twentieth Ed. 2020, the entire contents of which are hereby incorporated by reference. See also the National Cancer Institute (NCI) website (www.nci.nih.gov) and the Food and Drug Administration (FDA) website for a list of the FDA approved oncology drugs (www.fda.gov/cder/cancer/druglistframe).

In certain embodiments, the pharmaceutical compositions comprising the compounds disclosed herein further comprise one or more additional therapeutically active ingredients (e.g., chemotherapeutic and/or palliative). For purposes of the application, the term “palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

### Administration, Pharmaceutical Compositions

Administration of the disclosed compounds and pharmaceutical compositions can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride, and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes, and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles, and multilamellar vesicles. Liposomes can be formed from a variety of phospholipids, containing cholesterol, stearylamine or phosphatidylcholines.

In some embodiments, a film of lipid components is hydrated with an aqueous solution of drug to a form lipid layer encapsulating the drug, as described in U.S. Pat. No. 5,262,564, which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure is directed to pharmaceutical compositions comprising a compound of Formula I-A, Formula I-A′, or Formula I-B, and a pharmaceutically acceptable carrier. The pharmaceutically acceptable carrier may further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

In one embodiment, the disclosure provides a kit comprising two or more separate pharmaceutical compositions, at least one of which contains a compound of the present disclosure. In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

Pharmaceutical dosage forms of a compound of this disclosure may be manufactured by any of the methods well-known in the art, such as, for example, by conventional mixing, sieving, dissolving, melting, granulating, dragee-making, tableting, suspending, extruding, spray-drying, levigating, emulsifying, (nano-/micro-) encapsulating, entrapping, or lyophilization processes. As noted above, the compositions of this disclosure can include one or more physiologically acceptable inactive ingredients that facilitate processing of active molecules into preparations for pharmaceutical use.

As noted above, the compositions are comprised of, in general, a compound of this disclosure in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semi-solid excipients may be selected from glycerol, propylene glycol, water, ethanol and various oils, including those of petroleum, animal, vegetable or synthetic origin, e.g., peanut oil, soybean oil, mineral oil, sesame oil, etc. In some embodiments, liquid carriers, particularly for injectable solutions, include water, saline, aqueous dextrose, and glycols.

Compressed gases may be used to disperse a compound of this disclosure in an aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The compositions of this disclosure may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compositions comprising a compound of this disclosure that can be formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### FORMULATION EXAMPLES

The following are representative pharmaceutical formulations containing a compound of this disclosure.

### Formulation Example 1—Tablet Formulation

The following ingredients are mixed intimately and pressed into single scored tablets.

| | Quantity per |
|---|---|
| Ingredient | tablet, mg |
| compound of this disclosure | 400 |
| Cornstarch | 50 |
| croscarmellose sodium | 25 |
| Lactose | 120 |
| magnesium stearate | 5 |

### Formulation Example 2—Capsule Formulation

The following ingredients are mixed intimately and loaded into a hard-shell gelatin capsule

| | Quantity per |
|---|---|
| Ingredient | capsule, mg |
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| Flavoring | 0.035 | mL |
| Colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| Compound of this disclosure | 500 mg |
| Witepsol H-15 | balance |

### Dosing

The dosage regimen utilizing the disclosed compound is selected in accordance with a variety of factors including type, species, age, weight, sex, and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

The following synthetic and biological examples are offered to illustrate this disclosure and are not to be construed in any way as limiting the scope of this disclosure. Unless otherwise stated, all temperatures are in degrees Celsius.

### EXAMPLES

This disclosure is further understood by reference to the following examples, which are intended to be purely exemplary of this disclosure. This disclosure is not limited in scope by the exemplified embodiments, which are intended as illustrations of single aspects of this disclosure only. Any methods that are functionally equivalent are within the scope of this disclosure. Various modifications of this disclosure in addition to those described herein will become apparent to those skilled in the art from the foregoing description and accompanying figures. Such modifications fall within the scope of the appended claims.

In the specification and in the examples below, all temperatures are in degrees Celsius. In addition, the following abbreviations have the following meanings. If not defined, these abbreviations have their art recognized meaning.

| Abbreviation | Meaning |
|---|---|
| δ | chemical shift (ppm) |
| ACN or MeCN | acetonitrile |
| Boc | tert-butoxycarbonyl |
| BRET | Bioluminescence Resonance Energy Transfer |
| Cbz | benzyloxycarbonyl |
| DC₅₀ | concentration that resulted in a 50% |
| | targeted protein degradation |
| DCC | N,N-dicyclohexylcarbodiimide |
| DCM | dichloromethane |
| DIEA | diisopropylethylamine |
| DMA | dimethylacetamide |
| DMAP | 4-dimethylaminopyridine |
| DMF | N,N-dimethylformamide |
| DMP | Dess-Martin periodinane |
| DMSO | dimethylsulfoxide |
| d₆-DMSO | deuterated dimethylsulfoxide |
| dtbbpy | 4,4'-di-tert-butyl-2,2'-dipyridyl |
| EDCI | 1-ethyl-3-(3-dimethylaminopropyl)carbodiimide |
| eq. | equivalent(s) |
| ESI | electrospray ionization |
| EtOAc | ethyl acetate |
| EtOH | ethanol |
| FBS | fetal Bovine Serum |
| FITC | fluorescein isothiocyanate |
| Fmoc | fluorenylmethyloxycarbonyl |
| ¹H NMR | proton nuclear magnetic resonance spectroscopy |
| g | grams |
| h | hour(s) |
| HATU | 1-[bis(dimethylamino)methylene]-1H-1,2,3- |
| | triazolo[4,5-b]pyridinium 3-oxide |
| | hexafluorophosphate |
| HOBt | Hydroxybenzotriazole |
| HPLC | high performance liquid chromatography |
| IPA | isopropyl alcohol |
| Ir[(dF(CF₃)- | [4,4′-Bis(1,1-dimethylethyl)-2,2′-bipyridine- |
| ppy)₂dtbbpy]PF₆ | N1,N1′]bis[3,5-difluoro-2-[5-(trifluoromethyl)- |
| | 2-pyridinyl-N]phenyl-C]Iridium(III) |
| | hexafluorophosphate |
| JohnPhos | (2-biphenylyl)di-tert-butylphosphine |
| L | liter |
| LC | liquid chromatography |
| LC-MS | liquid chromatography-mass spectrometry |
| M | molar |
| mCPBA | meta-Chloroperoxybenzoic acid |
| MeOH | methanol |
| mg | milligram |
| mmol | millimole |
| mL | milliliter |
| umol or μmol | micromole |
| μM | micromolar |
| m/z | mass-to-charge ratio |
| min | minute(s) |
| N | normal |
| NiCl₂ glyme | Nickel(II) chloride ethylene glycol |
| | dimethyl ether complex |
| nm | nanometer |
| PBS | Phosphate-buffered saline |
| Pd(OAc)₂ | palladium (II) acetate |
| pM | picomolar |
| rt | room temperature |
| SEM | trimethylsilylethoxymethyl |
| SFC | supercritical fluid chromatography |
| SmI2 | Samarium Iodide |
| t-Bu | tert-butyl |
| TFA | trifluoroacetic acid |
| TFP | tri(2-furyl)phosphine |
| THF | tetrahydrofuran |
| TMP | 2,2,6,6-tetramethylpiperidine |
| TRITC | Tetramethylrhodamine |
| UV | ultraviolet |
| v/v | volume/volume ratio |
| NMR | br = broad |
| abbreviations | d = doublet |
| | dd = doublet of doublets |
| | ddt = doublet of doublet of triplets |
| | dtd = doublet of triplet of doublets |
| | m = multiplet |
| | qd = quartet of doublets |
| | quin = quintet |
| | s = singlet |
| | t = triplet |

### LC-MS Methods (General Method)

Method A: Experiments were performed using a Phenomenex Luna C18 150 mm×30 mm×5 μm, at a flow rate of 20 mL/min, and a mass spectrometer using ESI as ionization source. The solvent A was 4.0 mL of TFA in 4 L of water, and solvent B was 4.0 mL of TFA in 4 L of acetonitrile. The gradient consisted of 10-45% solvent B over 8 minutes, LC column temperature was 40° C. UV absorbance was collected at 220 nm and 254 nm.

Method B: Experiments were performed using a Waters Xbridge C18 150 mm×50 mm×10 μm, at a flow rate of 20 mL/min, and a mass spectrometer using ESI as ionization source. The solvent A was 4.0 mL of TFA in 4 L of water, and solvent B was 4.0 mL of TFA in 4 L of acetonitrile. The gradient consisted of 40-60% solvent B over 10 minutes, LC column temperature was 40° C. UV absorbance was collected at 220 nm and 254 nm.

### Example 1-A

### 3-(1-oxo-5-(((1S,2S)-2-(3-(tetrahydro-2H-pyran-4-yl)azetidin-1-yl)cyclohexyl)oxy)isoindolin-2-yl)piperidine-2,6-dione (Compound 1-A)

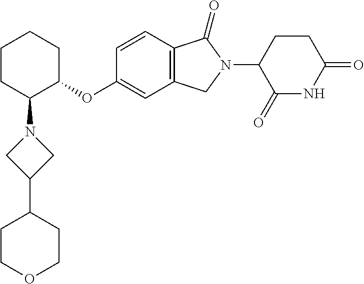 [SMILES: O=C1CCC(N2Cc3cc(O[C@H]4CCCC[C@@H]4N4CC(C5CCOCC5)C4)ccc3C2=O)C(=O)N1]

### Step 1:

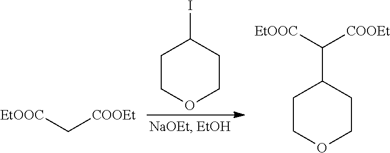 [SMILES: CCOC(=O)CC(=O)OCC ; IC1CCOCC1 ; CCOC(=O)C(C(=O)OCC)C1CCOCC1]

To a mixture of 4-iodotetrahydropyran (1.65 g, 7.80 mmol, 1.25 eq) and diethyl malonate (1 g, 6.24 mmol, 943.40 μL, 1 eq) in EtOH (10 mL) was added NaOEt (2.12 g, 6.24 mmol, 20% EtOH solution, 1 eq). The mixture was stirred at 78° C. for 16 h. The mixture was cooled to 20° C. and then concentrated under reduced pressure to remove EtOH. The residue was diluted with water (10 mL) and extracted with EtOAc (10 mL). The organic layer was washed with brine (5 mL), dried over Na₂SO₄ and concentrated under reduced pressure to give crude product. The crude product was purified by column chromatography (20% Petroleum ether in ethyl acetate) to give diethyl 2-(tetrahydro-2H-pyran-4-yl)malonate. ¹H NMR (400 MHz, CDCl₃) δ 1.27 (t, J=7.13 Hz, 6H), 1.43 (qd, J=12.38, 4.38 Hz, 2H), 1.59-1.68 (m, 2H), 2.25-2.39 (m, 1H), 3.17 (d, J=9.26 Hz, 1H), 3.36-3.49 (m, 2H), 3.95 (br dd, J=11.32, 3.81 Hz, 2H), 4.19 (q, J=7.05 Hz, 4H).

### Step 2:

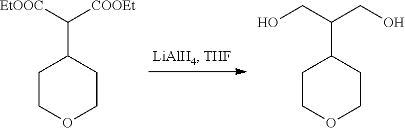 [SMILES: CCOC(=O)C(C(=O)OCC)C1CCOCC1 ; OCC(CO)C1CCOCC1]

To a suspension of LiAlH₄ (158.48 mg, 4.18 mmol, 3 eq) in THF (2 mL) was added diethyl 2-(tetrahydro-2H-pyran-4-yl)malonate (0.34 g, 1.39 mmol, 1 eq) at 0° C. The mixture was stirred at 0° C. for 1 h and then warmed to 20° C. The mixture was stirred at 20° C. for an additional 15 h. The mixture was cooled to 0° C. and then diluted with THF (4 mL). To the mixture was added water (0.16 mL) and a 15% aqueous NaOH solution (0.16 mL) and water (0.48 mL). The mixture was warmed to 20° C. and stirred for 1 h. MgSO₄ was added to the mixture, and the mixture was filtered. The filtrate was concentrated under vacuo to give crude product. The crude product was purified by column chromatography (10% MeOH in DCM) to give 2-(tetrahydro-2H-pyran-4-yl)propane-1,3-diol. ¹H NMR (400 MHz, CDCl₃) δ 1.32-1.42 (m, 2H), 1.49 (ddq, J=10.46, 6.89, 3.43 Hz, 1H), 1.60-1.68 (m, 2H), 1.74 (tdd, J=11.73, 7.82, 3.63 Hz, 1H), 2.58 (br s, 2H), 3.38 (td, J=11.82, 1.88 Hz, 2H), 3.77-3.84 (m, 2H), 3.84-3.90 (m, 2H), 3.97 (dd, J=11.32, 4.31 Hz, 2H).

### Step 3:

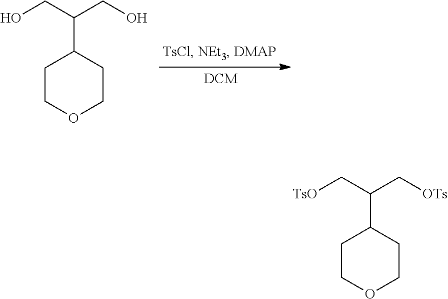 [SMILES: OCC(CO)C1CCOCC1 ; Cc1ccc(S(=O)(=O)OCC(COS(=O)(=O)c2ccc(C)cc2)C2CCOCC2)cc1]

To a solution of 2-(tetrahydro-2H-pyran-4-yl)propane-1,3-diol (100 mg, 624.18 μmol, 1 eq), TEA (189.48 mg, 1.87 mmol, 260.63 μL, 3 eq), and DMAP (15.25 mg, 124.84 μmol, 0.2 eq) in DCM (3 mL) was added TsCl (356.99 mg, 1.87 mmol, 3 eq). The mixture was stirred at 20° C. for 16 h. The mixture was diluted with water (2 mL) and extracted with DCM (3 mL). The organic layer was washed with brine (2 mL), dried over Na₂SO₄ and concentrated under vacuo to give crude product. The crude product was purified by column chromatography (3:1, v/v, Petroleum ether:EtOAc) to give 2-(tetrahydro-2H-pyran-4-yl)propane-1,3-diyl bis(4-methylbenzenesulfonate). ¹H NMR (400 MHz, CDCl₃) δ 0.78-0.91 (m, 1H), 1.23-1.27 (m, 3H), 1.59-1.66 (m, 1H), 1.73-1.82 (m, 1H), 2.46 (s, 6H), 3.25 (td, J=11.73, 1.53 Hz, 2H), 3.88 (dd, J=11.29, 3.84 Hz, 2H), 3.93-4.09 (m, 4H), 7.35 (d, J=8.33 Hz, 4H), 7.73 (d, J=8.11 Hz, 4H).

### Step 4:

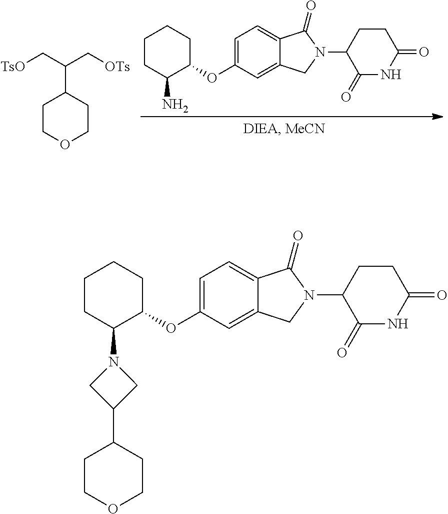

A mixture of 3-(5-(((1S,2S)-2-aminocyclohexyl)oxy)-1-oxoisoindolin-2-yl)piperidine-2,6-dione (60 mg, 167.88 μmol, 1 eq) [prepared according to literature procedure described in PCT Int. Appl. WO2020012334], 2-(tetrahydro-2H-pyran-4-yl)propane-1,3-diyl bis(4-methylbenzenesulfonate) (102.26 mg, 218.24 μmol, 1.3 eq) and DIEA (108.49 mg, 839.39 μmol, 146.21 μL, 5 eq) were taken up into a microwave tube in MeCN (2 mL). The sealed tube was heated at 120° C. for 24 h. The mixture was filtered. The filtrate was collected. The filtrate was purified by prep-HPLC (method B) and lyophilized to give 3-(1-oxo-5-(((1S,2S)-2-(3-(tetrahydro-2H-pyran-4-yl)azetidin-1-yl)cyclohexyl)oxy)isoindolin-2-yl)piperidine-2,6-dione. ¹H NMR (400 MHz, d₄-MeOH) δ 1.09-1.31 (m, 2H), 1.32-1.72 (m, 6H), 1.74-1.98 (m, 3H), 2.11-2.27 (m, 2H), 2.28-2.38 (m, 1H), 2.39-2.55 (m, 1H), 2.57-2.71 (m, 1H), 2.72-2.83 (m, 1H), 2.84-2.99 (m, 1H), 3.34-3.45 (m, 2H), 3.46-3.66 (m, 1H), 3.86-3.97 (m, 2H), 3.97-4.25 (m, 2H), 4.25-4.43 (m, 1H), 4.43-4.68 (m, 3H), 5.07-5.19 (m, 1H), 7.15 (br d, J=8.44 Hz, 1H), 7.23 (br s, 1H), 7.76 (br d, J=8.31 Hz, 1H).

### Example 2-A

### Preparation of 3-(1-oxo-5-((2-oxocyclohexyl)oxy)isoindolin-2-yl)-1-((2-(trimethylsilyl)ethoxy)methyl)piperidine-2,6-dione

### Step 1:

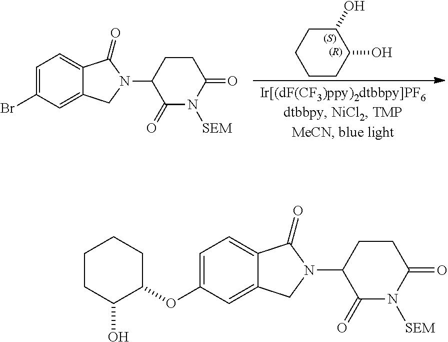

To a mixture of 3-(5-bromo-1-oxoisoindolin-2-yl)-1-((2-(trimethylsilyl)ethoxy)methyl)piperidine-2,6-dione (3.84 g, 33.08 mmol, 1.5 eq) [prepared according to literature procedure described in PCT Int. Appl. WO2020012334], (1R,2S)-cyclohexane-1,2-diol (2.55 g, 22.0 mmol, 1.0 eq), dtbbpy (295.98 mg, 1.10 mmol, 0.05 eq), Ir[(dF(CF₃)ppy)₂dtbbpy]PF₆ (247.44 mg, 220.56 μmol, 0.01 eq), and NiCl₂·glyme (242.30 mg, 1.10 mmol, 0.05 eq) in CH₃CN (100 mL), was added TMP (3.74 g, 26.47 mmol, 4.49 mL, 1.2 eq). The reaction mixture was stirred at 25° C. for 12 hrs. The reaction mixture was filtered and then concentrated in vacuum. The residue was purified by column chromatography (50-100% ethyl acetate in petroleum) to give 3-(5-(((1S,2R)-2-hydroxycyclohexyl)oxy)-1-oxoisoindolin-2-yl)-1-((2-(trimethylsilyl)ethoxy)methyl)piperidine-2,6-dione. ¹H NMR (400 MHz, d₆-DMSO) δ 7.65-7.57 (m, 1H), 7.19 (s, 1H), 7.07 (dd, J=2.1, 8.4 Hz, 1H), 5.18 (dd, J=5.0, 13.4 Hz, 1H), 5.05 (q, J=9.7 Hz, 2H), 4.94 (dd, J=1.2, 4.7 Hz, 1H), 4.47 (d, J=3.6 Hz, 1H), 4.40 (dd, J=4.9, 17.1 Hz, 1H), 4.26-4.12 (m, 2H), 3.61-3.46 (m, 3H), 3.14-2.99 (m, 2H), 2.78 (br dd, J=2.1, 15.6 Hz, 1H), 2.43-2.28 (m, 1H), 2.07-2.01 (m, 2H), 1.94-1.82 (m, 1H), 1.79-1.69 (m, 1H), 1.63 (br d, J=9.6 Hz, 2H), 1.58-1.53 (m, 1H), 1.37-1.27 (m, 3H), 1.13 (br d, J=7.9 Hz, 1H), 0.90-0.78 (m, 2H), 0.02 (s, 9H).

### Step 2:

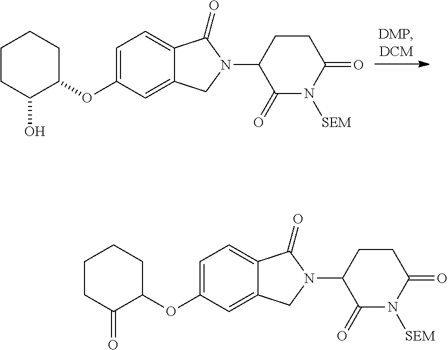 [SMILES: C[Si](C)(C)CCOCN1C(=O)CCC(N2Cc3cc(O[C@H]4CCCC[C@H]4O)ccc3C2=O)C1=O ; C[Si](C)(C)CCOCN1C(=O)CCC(N2Cc3cc(OC4CCCCC4=O)ccc3C2=O)C1=O]

To a mixture of 3-(5-(((1S,2R)-2-hydroxycyclohexyl)oxy)-1-oxoisoindolin-2-yl)-1-((2-(trimethylsilyl)ethoxy)methyl)piperidine-2,6-dione (5 g, 10.23 mmol, 1 eq) in DCM (50 mL), was added DMP (8.68 g, 20.46 mmol, 6.34 mL, 2 eq). The mixture was stirred at 25° C. for 2 hrs. The reaction mixture was filtered and concentrated in vacuum. The residue was purified by column chromatography (50-100% Petroleum ether in Ethyl acetate) to give 3-(1-oxo-5-((2-oxocyclohexyl)oxy)isoindolin-2-yl)-1-((2-(trimethylsilyl)ethoxy)methyl)piperidine-2,6-dione. ¹H NMR (400 MHz, d₆-DMSO) δ 7.59 (d, J=8.4 Hz, 1H), 7.07 (s, 1H), 7.00 (dd, J=1.2, 8.4 Hz, 1H), 5.25-5.14 (m, 2H), 5.09-4.97 (m, 2H), 4.38 (dd, J=5.0, 17.0 Hz, 1H), 4.25-4.15 (m, 1H), 3.72-3.42 (m, 2H), 3.16-3.00 (m, 1H), 2.87-2.73 (m, 1H), 2.71-2.58 (m, 1H), 2.40-2.28 (m, 3H), 2.10-1.99 (m, 2H), 1.93-1.74 (m, 3H), 1.66-1.51 (m, 1H), 0.88-0.79 (m, 2H), 0.02 (d, J=1.4 Hz, 9H).

### Example 2-A′

### Preparation of 3-(1-oxo-5-((2-oxocyclohexyl)oxy)isoindolin-2-yl)-1-((2-(trimethylsilyl)ethoxy)methyl)piperidine-2,6-dione

### Step 1:

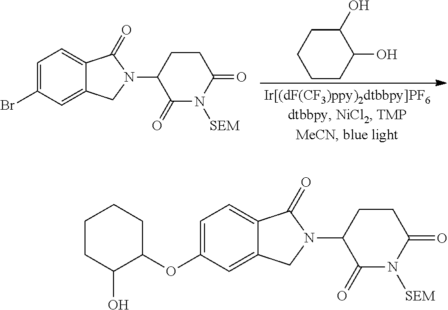 [SMILES: C[Si](C)(C)CCOCN1C(=O)CCC(N2Cc3cc(Br)ccc3C2=O)C1=O ; OC1CCCCC1O ; C[Si](C)(C)CCOCN1C(=O)CCC(N2Cc3cc(OC4CCCCC4O)ccc3C2=O)C1=O]

To a mixture of 3-(5-bromo-1-oxoisoindolin-2-yl)-1-((2-(trimethylsilyl)ethoxy)methyl)piperidine-2,6-dione (19.9 g, 145.6 mmol, 1.5 eq) [prepared according to literature procedure described in PCT Int. Appl. WO2020012334], cyclohexane-1,2-diol (11.3 g, 97.1 mmol, 1.0 eq.), dtbbpy (1.30 g, 4.40 mmol, 0.05 eq), Ir[(dF(CF₃)ppy)₂dtbbpy]PF₆ (1090 mg, 882 μmol, 0.01 eq), and NiCl₂·glyme (1065 mg, 4.40 mmol, 0.05 eq) in CH₃CN (500 mL), was added TMP (16.56 g, 166.47 mmol, 1.2 eq). The reaction mixture was stirred at 25° C. for 12 hrs. The reaction mixture was filtered and then concentrated in vacuum. The residue was purified by column chromatography (50-100% ethyl acetate in petroleum ether) to give 3-(5-((2-hydroxycyclohexyl)oxy)-1-oxoisoindolin-2-yl)-1-((2-(trimethylsilyl)ethoxy)methyl)piperidine-2,6-dione. ¹H NMR (400 MHz, d₆-DMSO) δ 7.65-7.57 (m, 1H), 7.19 (s, 1H), 7.07 (dd, J=2.1, 8.4 Hz, 1H), 5.18 (dd, J=5.0, 13.4 Hz, 1H), 5.05 (q, J=9.7 Hz, 2H), 4.94 (dd, J=1.2, 4.7 Hz, 1H), 4.47 (d, J=3.6 Hz, 1H), 4.40 (dd, J=4.9, 17.1 Hz, 1H), 4.26-4.12 (m, 2H), 3.61-3.46 (m, 3H), 3.14-2.99 (m, 2H), 2.78 (br dd, J=2.1, 15.6 Hz, 1H), 2.43-2.28 (m, 1H), 2.07-2.01 (m, 2H), 1.94-1.82 (m, 1H), 1.79-1.69 (m, 1H), 1.63 (br d, J=9.6 Hz, 2H), 1.58-1.53 (m, 1H), 1.37-1.27 (m, 3H), 1.13 (br d, J=7.9 Hz, 1H), 0.90-0.78 (m, 2H), 0.02 (s, 9H).

One of skill in the art would be able to separate and isolate the individual stereoisomers of the 3-(5-((2-hydroxycyclohexyl)oxy)-1-oxoisoindolin-2-yl)-1-((2-(trimethylsilyl)ethoxy)methyl)piperidine-2,6-dione product reported, using techniques known in the art.

### Step 2:

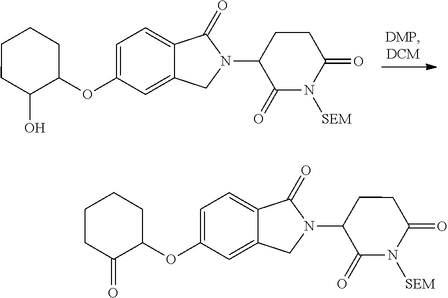 [SMILES: C[Si](C)(C)CCOCN1C(=O)CCC(N2Cc3cc(OC4CCCCC4O)ccc3C2=O)C1=O ; C[Si](C)(C)CCOCN1C(=O)CCC(N2Cc3cc(OC4CCCCC4=O)ccc3C2=O)C1=O]

To a mixture of 3-(5-((2-hydroxycyclohexyl)oxy)-1-oxoisoindolin-2-yl)-1-((2-(trimethylsilyl)ethoxy)methyl)piperidine-2,6-dione (20 g, 40.9 mmol, 1 eq) in DCM (200 mL), was added DMP (34.72 g, 81.84 mmol, 2 eq). The mixture was stirred at 25° C. for 2 hrs. The reaction mixture was filtered and concentrated in vacuum. The residue was purified by column chromatography (50-100% ethyl acetate in petroleum ether) to give 3-(1-oxo-5-((2-oxocyclohexyl)oxy)isoindolin-2-yl)-1-((2-(trimethylsilyl)ethoxy)methyl)piperidine-2,6-dione. ¹H NMR (400 MHz, d₆-DMSO) δ 7.59 (d, J=8.4 Hz, 1H), 7.07 (s, 1H), 7.00 (dd, J=1.2, 8.4 Hz, 1H), 5.25-5.14 (m, 2H), 5.09-4.97 (m, 2H), 4.38 (dd, J=5.0, 17.0 Hz, 1H), 4.25-4.15 (m, 1H), 3.72-3.42 (m, 2H), 3.16-3.00 (m, 1H), 2.87-2.73 (m, 1H), 2.71-2.58 (m, 1H), 2.40-2.28 (m, 3H), 2.10-1.99 (m, 2H), 1.93-1.74 (m, 3H), 1.66-1.51 (m, 1H), 0.88-0.79 (m, 2H), 0.02 (d, J=1.4 Hz, 9H).

One of skill in the art would be able to separate and isolate the individual stereoisomers of the 3-(1-oxo-5-((2-oxocyclohexyl)oxy)isoindolin-2-yl)-1-((2-(trimethylsilyl)ethoxy)methyl)piperidine-2,6-dione product reported, using techniques known in the art.

### Example 3-A

### Synthesis of isomers of 3-(1-oxo-5-((2-(3-(piperidine-1-carbonyl)azetidin-1-yl)cyclohexyl)oxy)isoindolin-2-yl)piperidine-2,6-dione (Compounds 22-A and 23-A)

### Step 1:

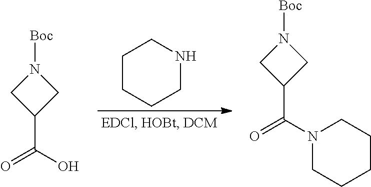 [SMILES: CC(C)(C)OC(=O)N1CC(C(=O)O)C1 ; C1CCNCC1 ; CC(C)(C)OC(=O)N1CC(C(=O)N2CCCCC2)C1]

To a stirred solution of 1-(tert-butoxycarbonyl)azetidine-3-carboxylic acid (1 g, 4.97 mmol, 1 eq), EDCI (1.14 g, 5.96 mmol, 1.2 eq), HOBt (805.83 mg, 5.96 mmol, 1.2 eq), and DIEA (1.93 g, 14.91 mmol, 2.60 mL, 3 eq) in DCM (10 mL), was added piperidine (507.79 mg, 5.96 mmol, 588.94 μL, 1.2 eq). The mixture was stirred at 20° C. for 1 h. The reaction mixture was diluted with water (10 mL), extracted with DCM (20 mL×2), washed with brine (10 mL×3), dried over Na₂SO₄, filtered, and the solvents evaporated in vacuo. The residue was purified by column chromatography (SiO₂, 0 to 25% ethyl acetate in petroleum ether) to give tert-butyl 3-(piperidine-1-carbonyl)azetidine-1-carboxylate. ¹H NMR (400 MHz, CDCl₃) δ 1.43 (s, 9H), 1.49-1.60 (m, 4H), 1.61-1.71 (m, 2H), 3.13-3.26 (m, 2H), 3.40-3.50 (m, 1H), 3.54-3.62 (m, 2H), 4.04 (t, J=8.57 Hz, 2H), 4.11-4.26 (m, 2H).

### Step 2:

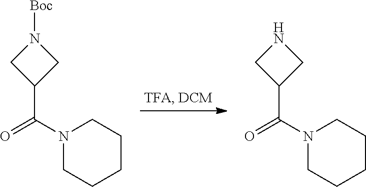 [SMILES: CC(C)(C)OC(=O)N1CC(C(=O)N2CCCCC2)C1 ; O=C(C1CNC1)N1CCCCC1]

To a solution of tert-butyl 3-(piperidine-1-carbonyl)azetidine-1-carboxylate (0.2 g, 745.29 μmol, 1 eq) in DCM (1.5 mL), was added TFA (770.00 mg, 6.75 mmol, 0.5 mL, 9.06 eq). The mixture was stirred at 20° C. for 2 h. The solvents evaporated in vacuo to give azetidin-3-yl(piperidin-1-yl)methanone, which was used or next step without any purification. m/z (ESI⁺) 169.3 (M+H)⁺.

### Step 3:

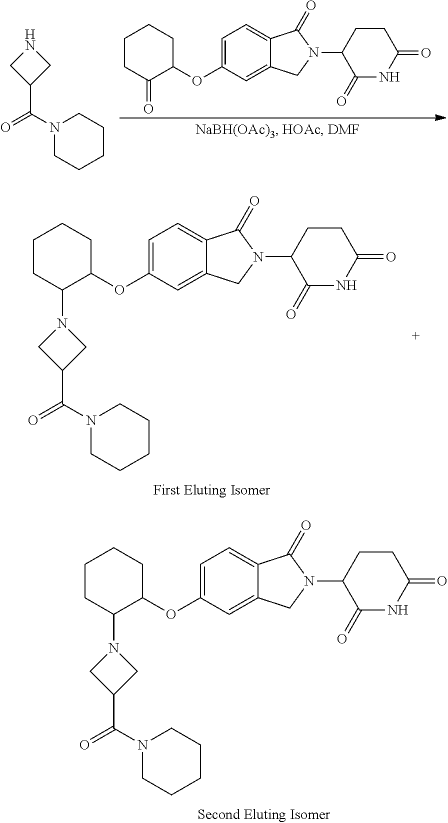 [SMILES: O=C(C1CNC1)N1CCCCC1 ; O=C1CCC(N2Cc3cc(OC4CCCCC4=O)ccc3C2=O)C(=O)N1 ; O=C1CCC(N2Cc3cc(OC4CCCCC4N4CC(C(=O)N5CCCCC5)C4)ccc3C2=O)C(=O)N1 ; O=C1CCC(N2Cc3cc(OC4CCCCC4N4CC(C(=O)N5CCCCC5)C4)ccc3C2=O)C(=O)N1]

To a solution of 3-(1-oxo-5-((2-oxocyclohexyl)oxy)isoindolin-2-yl)piperidine-2,6-dione (50 mg, 140.30 μmol, 1 eq) and azetidin-3-yl(piperidin-1-yl)methanone (59.40 mg, 210.45 μmol, 1.5 eq, TFA) in DMF (1 mL), was added NaBH(OAc)₃ (59.47 mg, 280.61 μmol, 2 eq) and AcOH (12.64 mg, 210.45 μmol, 12.04 μL, 1.5 eq). The mixture was stirred at 20° C. for 12 h. Water (1 mL) was added and the reaction was filtered and the solvents evaporated in vacuo. The residue was purified by prep-HPLC (method B) to give two diastereoisomers.

First eluting isomer of 3-(1-oxo-5-((2-(3-(piperidine-1-carbonyl)azetidin-1-yl)cyclohexyl)oxy)isoindolin-2-yl)piperidine-2,6-dione, Compound 23-A: ¹H NMR (400 MHz, d₄-methanol) δ 1.35-1.58 (m, 8H), 1.63-1.82 (m, 3H), 1.89 (br d, J=9.38 Hz, 2H), 2.12-2.24 (m, 2H), 2.48 (qd, J=13.20, 4.69 Hz, 1H), 2.74-2.82 (m, 1H), 2.85-2.97 (m, 1H), 3.20-3.30 (m, 2H), 3.50-3.58 (m, 2H), 3.78-3.91 (m, 1H), 4.02-4.23 (m, 4H), 4.38-4.53 (m, 2H), 4.89-4.95 (m, 2H), 5.09-5.16 (m, 1H), 7.17-7.22 (m, 1H), 7.25 (s, 1H), 7.75 (d, J=8.50 Hz, 1H).

Second eluting isomer of 3-(1-oxo-5-((2-(3-(piperidine-1-carbonyl)azetidin-1-yl)cyclohexyl)oxy)isoindolin-2-yl)piperidine-2,6-dione, Compound 22-A: ¹H NMR (400 MHz, d₄-methanol) δ 1.16-1.31 (m, 1H), 1.33-1.58 (m, 7H), 1.62-1.71 (m, 2H), 1.77-1.87 (m, 2H), 2.03-2.26 (m, 3H), 2.48 (qd, J=13.20, 4.69 Hz, 1H), 2.74-2.82 (m, 1H), 2.85-2.96 (m, 1H), 2.97-3.05 (m, 1H), 3.29 (br d, J=5.75 Hz, 2H), 3.50-3.57 (m, 2H), 3.70 (quin, J=8.04 Hz, 1H), 3.88 (br t, J=8.13 Hz, 1H), 3.94-4.08 (m, 3H), 4.35-4.52 (m, 3H), 5.07-5.15 (m, 1H), 7.10 (dd, J=8.50, 1.88 Hz, 1H), 7.19 (s, 1H), 7.73 (d, J=8.38 Hz, 1H).

### Example 4-A

### Synthesis of tert-butyl 4-(1-((1S,2S)-2-((2-(2,6-dioxopiperidin-3-yl)-1-oxoisoindolin-5-yl)oxy)cyclohexyl)azetidin-3-yl)piperidine-1-carboxylate (Compound 26-A)

### Step 1:

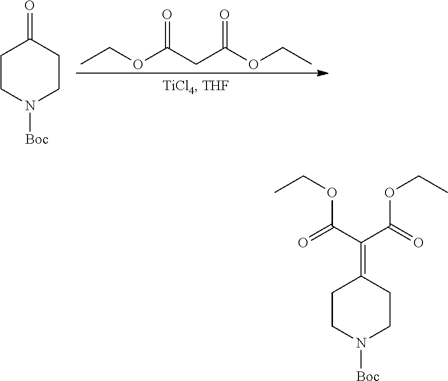

A solution of TiCl₄ (7.14 g, 37.64 mmol, 2.5 eq) in CCl₄ (3 mL) was added to THF (12 mL) at 0° C. A solution tert-butyl 4-oxopiperidine-1-carboxylate (3 g, 15.06 mmol, 1 eq) and diethyl propanedioate (2.41 g, 15.06 mmol, 2.28 mL, 1 eq) in THF (15 mL) was added dropwise at 0° C. and stirred for 15 min. Pyridine (2.98 g, 37.64 mmol, 3.04 mL, 2.5 eq) was added dropwise at 0° C. and the resulting mixture was stirred for 12 h at 25° C. The reaction mixture was diluted with H₂O (20 mL) and extracted with ethyl acetate (15 mL×3). The combined organic layers were washed with saturated brine (5 mL×5), dried over Na₂SO₄, filtered, and the solvents evaporated in vacuo. The residue was purified by column chromatography (SiO₂, 10 to 25% ethyl acetate in petroleum ether) to give diethyl 2-(1-(tert-butoxycarbonyl)piperidin-4-ylidene)malonate. ¹H NMR (400 MHz, CDCl₃) δ 1.30 (t, J=7.13 Hz, 6H), 1.47 (s, 9H), 2.62-2.69 (m, 4H), 3.53 (t, J=5.82 Hz, 4H), 4.25 (q, J=7.13 Hz, 4H).

### Step 2:

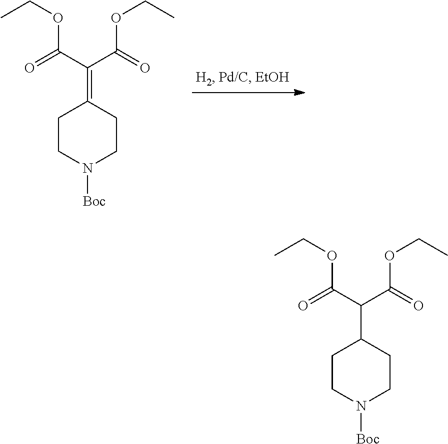 [SMILES: CCOC(=O)C(C(=O)OCC)=C1CCN(C(=O)OC(C)(C)C)CC1 ; CCOC(=O)C(C(=O)OCC)C1CCN(C(=O)OC(C)(C)C)CC1]

To a solution of diethyl 2-(1-(tert-butoxycarbonyl)piperidin-4-ylidene)malonate (2 g, 5.86 mmol, 1 eq) in EtOH (20 mL), was added 10% Pd/C (0.6 g). The suspension was degassed and purged with H₂ three times. The mixture was stirred under a H₂ atmosphere (15 psi) for 12 h. The reaction mixture was filtered and concentrated under reduced pressure to give diethyl 2-(1-(tert-butoxycarbonyl)piperidin-4-yl)malonate, which was used in the next step without further purification. ¹H NMR (400 MHz, CDCl₃) δ 1.27 (t, J=7.13 Hz, 8H), 1.44 (s, 9H), 1.68 (br d, J=12.88 Hz, 2H), 2.16-2.29 (m, 1H), 2.72 (br t, J=10.76 Hz, 2H), 3.16 (d, J=9.01 Hz, 1H), 3.99-4.15 (m, 2H), 4.19 (q, J=7.13 Hz, 4H).

### Step 3:

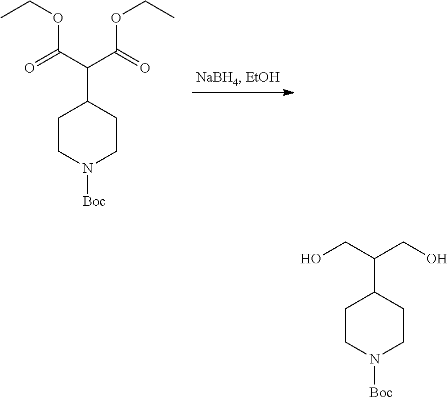 [SMILES: CCOC(=O)C(C(=O)OCC)C1CCN(C(=O)OC(C)(C)C)CC1 ; CC(C)(C)OC(=O)N1CCC(C(CO)CO)CC1]

To a solution of diethyl 2-(1-(tert-butoxycarbonyl)piperidin-4-yl)malonate (2 g, 5.82 mmol, 1 eq) in EtOH (20 mL), was added NaBH₄ (2.20 g, 58.24 mmol, 10 eq) at 0° C. The mixture was slowly warmed to 25° C. and stirred for 2 hours. The reaction was cooled to 0° C. and saturated NH₄Cl solution (10 mL) was added, then the reaction was diluted with H₂O (20 mL) and extracted with ethyl acetate (15 mL×3). The combined organic layers were washed with saturated brine (15 mL×3), dried over Na₂SO₄, filtered and concentrated under reduced pressure to give tert-butyl 4-(1,3-dihydroxypropan-2-yl)piperidine-1-carboxylate, which was used into the next step without further purification. ¹H NMR (400 MHz, CDCl₃) δ 1.14-1.23 (m, 2H), 1.45 (s, 9H), 1.49-1.56 (m, 1H), 1.60-1.76 (m, 3H), 2.54-2.75 (m, 4H), 3.75-3.93 (m, 4H), 4.06-4.20 (m, 2H).

### Step 4:

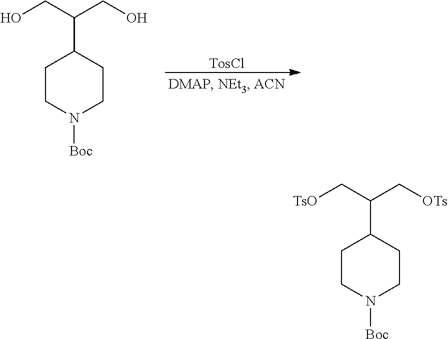

To a solution of tert-butyl 4-(1,3-dihydroxypropan-2-yl)piperidine-1-carboxylate (400 mg, 1.54 mmol, 1 eq) in ACN (5 mL), was added TsCl (1.03 g, 5.40 mmol, 3.5 eq), TEA (624.28 mg, 6.17 mmol, 858.71 μL, 4 eq), and DMAP (376.86 mg, 3.08 mmol, 2 eq). The mixture was stirred at 25° C. for 3 h. The reaction mixture was concentrated under reduced pressure. The residue was purified by column chromatography (SiO₂, 20 to 25% ethyl acetate in petroleum ether) to give tert-butyl 4-(1,3-bis(tosyloxy)propan-2-yl)piperidine-1-carboxylate. ¹H NMR (400 MHz, CDCl₃) δ 1.00-1.13 (m, 2H), 1.44 (s, 9H), 1.47-1.59 (m, 2H), 1.51-1.61 (m, 1H), 1.76-1.85 (m, 1H), 2.47 (s, 6H), 2.51-2.66 (m, 2H), 3.92-3.99 (m, 2H), 4.00-4.09 (m, 4H), 7.37 (d, J=8.13 Hz, 4H), 7.75 (d, J=8.25 Hz, 4H).

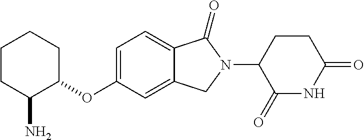 [SMILES: N[C@H]1CCCC[C@@H]1Oc1ccc2c(c1)CN(C1CCC(=O)NC1=O)C2=O]

This intermediate was prepared according to reported literature procedure [ADCOCK, Claire et al., US2020/17461A1].

### Step 5:

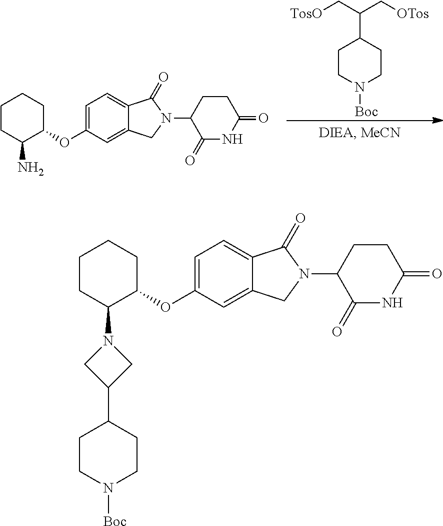

To a solution of 3-(5-(((1S,2S)-2-aminocyclohexyl)oxy)-1-oxoisoindolin-2-yl)piperidine-2,6-dione (50 mg, 139.90 μmol, 1 eq), tert-butyl 4-(1,3-bis(tosyloxy)propan-2-yl)piperidine-1-carboxylate (119.13 mg, 209.85 μmol, 1.5 eq) in MeCN (2 mL), was added DIEA (90.40 mg, 699.49 μmol, 121.84 μL, 5 eq). The mixture was stirred at 120° C. for 12 h. The reaction mixture was concentrated under reduced pressure. The residue was purified by prep-HPLC (method B) to give tert-butyl 4-(1-((1S,2S)-2-((2-(2,6-dioxopiperidin-3-yl)-1-oxoisoindolin-5-yl)oxy)cyclohexyl)azetidin-3-yl)piperidine-1-carboxylate. ¹H NMR (400 MHz, d₄-Methanol) δ 0.86-1.00 (m, 2H), 1.03-1.15 (m, 1H), 1.25-1.40 (m, 3H), 1.43 (s, 9H), 1.50-1.64 (m, 3H), 1.76 (br s, 2H), 1.96 (br d, J=11.26 Hz, 1H), 2.09-2.21 (m, 3H), 2.47 (qd, J=13.05, 4.75 Hz, 2H), 2.62-2.82 (m, 3H), 2.85-2.92 (m, 1H), 2.93-3.01 (m, 1H), 3.19 (br t, J=8.00 Hz, 1H), 3.51 (q, J=7.42 Hz, 2H), 4.04 (br d, J=13.01 Hz, 2H), 4.21-4.30 (m, 1H), 4.37-4.50 (m, 2H), 4.59 (br s, 1H), 5.11 (dd, J=13.26, 5.13 Hz, 1H), 7.05 (dd, J=8.38, 1.88 Hz, 1H), 7.13 (s, 1H), 7.71 (d, J=8.38 Hz, 1H).

### Example 5-A

### Synthesis 3-(5-(((1S,2S)-2-(3-(1-acetylpiperidin-4-yl)azetidin-1-yl)cyclohexyl)oxy)-1-oxoisoindolin-2-yl)piperidine-2,6-dione (Compound 12-A)

### Step 1:

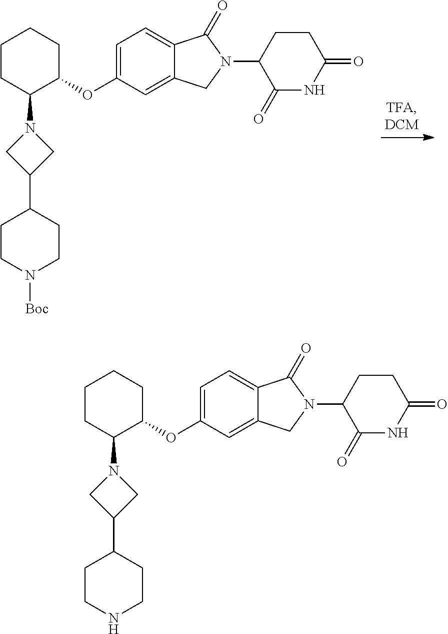 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1 ; O=C1CCC(N2Cc3cc(O[C@H]4CCCC[C@@H]4N4CC(C5CCNCC5)C4)ccc3C2=O)C(=O)N1]

To a solution of tert-butyl 4-(1-((1S,2S)-2-((2-(2,6-dioxopiperidin-3-yl)-1-oxoisoindolin-5-yl)oxy)cyclohexyl)azetidin-3-yl)piperidine-1-carboxylate (70 mg, 120.54 μmol, 1 eq) in DCM (1 mL), was added TFA (412.33 mg, 3.62 mmol, 267.75 μL, 30 eq). The mixture was stirred at 25° C. for 2 h. The reaction mixture was concentrated under reduced pressure to give 3-(1-oxo-5-(((1S,2S)-2-(3-(piperidin-4-yl)azetidin-1-yl)cyclohexyl)oxy)isoindolin-2-yl)piperidine-2,6-dione, which was used in the next step without further purification. ¹H NMR (400 MHz, d₄-Methanol) δ 1.24-1.53 (m, 6H), 1.79-2.04 (m, 5H), 2.12-2.23 (m, 2H), 2.30 (br d, J=10.76 Hz, 1H), 2.41-2.55 (m, 1H), 2.63-2.84 (m, 2H), 2.84-3.03 (m, 3H), 3.40 (br d, J=11.76 Hz, 2H), 3.49 (br d, J=8.25 Hz, 1H), 4.01-4.12 (m, 1H), 4.14-4.32 (m, 2H), 4.38-4.53 (m, 2H), 4.58 (br s, 1H), 5.12 (dd, J=13.26, 5.13 Hz, 1H), 7.14 (br d, J=8.38 Hz, 1H), 7.23 (br s, 1H), 7.74 (d, J=8.25 Hz, 1H).

### Step 2:

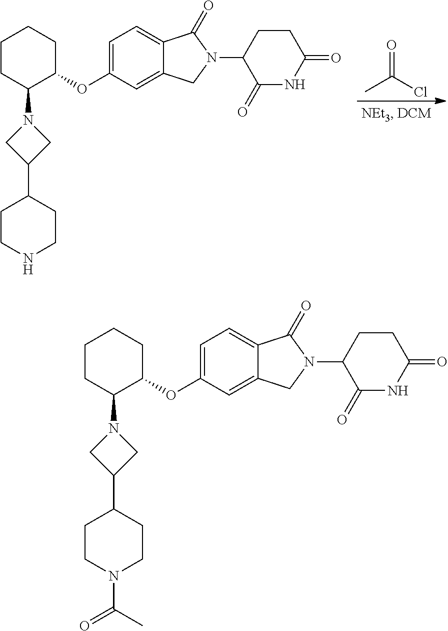

To a solution of 3-(1-oxo-5-(((1S,2S)-2-(3-(piperidin-4-yl)azetidin-1-yl)cyclohexyl)oxy)isoindolin-2-yl)piperidine-2,6-dione (25 mg, 52.02 μmol, 1 eq) and acetyl chloride (7.35 mg, 93.63 μmol, 6.68 μL, 1.8 eq) in DCM (0.3 mL), was added TEA (10.53 mg, 104.04 μmol, 14.48 μL, 2 eq). The mixture was stirred at 25° C. for 12 h. The reaction mixture was concentrated under reduced pressure. The residue was purified by prep-HPLC (method A) to give 3-(5-(((1S,2S)-2-(3-(1-acetylpiperidin-4-yl)azetidin-1-yl)cyclohexyl)oxy)-1-oxoisoindolin-2-yl)piperidine-2,6-dione. ¹H NMR (400 MHz, d₄-Methanol) δ 0.90-1.14 (m, 2H), 1.26-1.53 (m, 4H), 1.69 (br t, J=14.76 Hz, 2H), 1.76-1.92 (m, 3H), 2.07 (s, 3H), 2.16 (ddt, J=9.97, 5.13, 2.58 Hz, 2H), 2.27 (br d, J=11.01 Hz, 1H), 2.41-2.65 (m, 3H), 2.74-2.83 (m, 1H), 2.85-2.97 (m, 1H), 3.02-3.14 (m, 1H), 3.79-4.16 (m, 5H), 4.38-4.58 (m, 4H), 5.12 (dd, J=13.38, 5.13 Hz, 1H), 7.09-7.27 (m, 2H), 7.74 (d, J=8.38 Hz, 1H).

### Example 6-A

### Synthesis 3-(5-(((1S,2S)-2-(3-(1-(1-methylcyclobutane-1-carbonyl)piperidin-4-yl)azetidin-1-yl)cyclohexyl)oxy)-1-oxoisoindolin-2-yl)piperidine-2,6-dione (Compound 25-A)

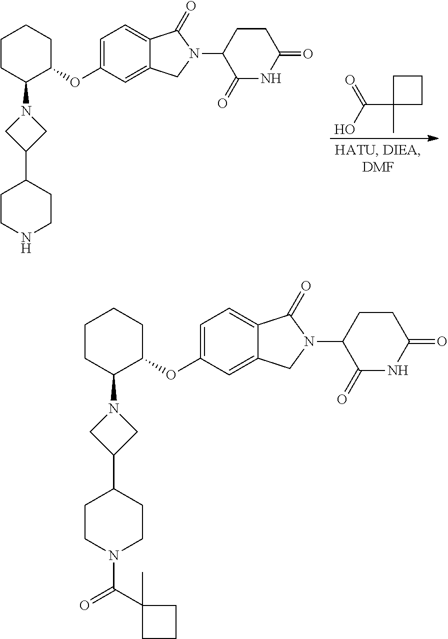

To a solution of 3-(1-oxo-5-(((1S,2S)-2-(3-(piperidin-4-yl)azetidin-1-yl)cyclohexyl)oxy)isoindolin-2-yl)piperidine-2,6-dione (25 mg, 52.02 μmol, 1 eq) in DMF (0.3 mL), was added 1-methylcyclobutanecarboxylic acid (10.69 mg, 93.64 μmol, 10.88 μL, 1.8 eq), DIEA (33.62 mg, 260.10 μmol, 45.30 μL, 5 eq), and HATU (29.67 mg, 78.03 μmol, 1.5 eq). The reaction was stirred at 25° C. for 12 h. The reaction mixture was concentrated under reduced pressure. The residue was purified by prep-HPLC (method A) to give 3-(5-(((1S,2S)-2-(3-(1-(1-methylcyclobutane-1-carbonyl)piperidin-4-yl)azetidin-1-yl)cyclohexyl)oxy)-1-oxoisoindolin-2-yl)piperidine-2,6-dione. ¹H NMR (400 MHz, d₄-Methanol) δ 0.90-1.09 (m, 2H), 1.16-1.30 (m, 1H), 1.33-1.48 (m, 6H), 1.63-1.72 (m, 3H), 1.72-1.78 (m, 1H), 1.78-1.92 (m, 4H), 1.92-2.04 (m, 1H), 2.10 (br d, J=11.13 Hz, 1H), 2.16 (dtd, J=12.69, 5.25, 2.44 Hz, 1H), 2.23 (br d, J=10.26 Hz, 1H), 2.34-2.53 (m, 4H), 2.53-2.63 (m, 1H), 2.74-2.82 (m, 1H), 2.85-2.93 (m, 1H), 2.93-3.08 (m, 2H), 3.54 (br s, 1H), 3.63-3.77 (m, 2H), 3.89 (br d, J=7.25 Hz, 2H), 4.35-4.52 (m, 4H), 5.12 (dd, J=13.26, 5.00 Hz, 1H), 7.10 (br d, J=8.38 Hz, 1H), 7.18 (s, 1H), 7.73 (d, J=8.38 Hz, 1H).

### Example 1-B (Method 1-B)

### Preparation of 3-(1-oxo-5-((2-(3-(2-(trifluoromethyl)pyridin-4-yl)azetidin-1-yl)cyclohexyl)oxy)isoindolin-2-yl)piperidine-2,6-dione (Compound 1-B)

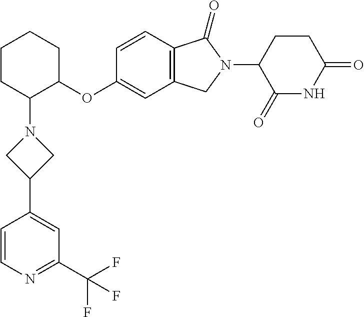 [SMILES: O=C1CCC(N2Cc3cc(OC4CCCCC4N4CC(c5ccnc(C(F)(F)F)c5)C4)ccc3C2=O)C(=O)N1]

### Step 1:

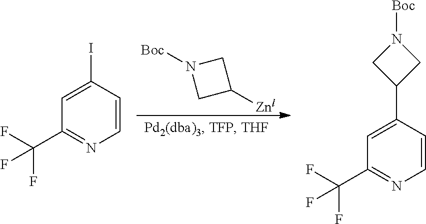

To a solution of 4-iodo-2-(trifluoromethyl)pyridine (2.89 g, 10.60 mmol, 1.00 eq) and (1-tert-butoxycarbonylazetidin-3-yl)-iodo-zinc (5.54 g, 15.90 mmol, 1.5 eq) in THF (30 mL), was added Pd₂(dba)₃ (194.09 mg, 211.96 umol, 0.02 eq) and TFP (246.05 mg, 1.06 mmol, 0.10 eq) under a N₂ atmosphere. The reaction mixture was stirred at 25° C. for 12 h. The reaction mixture was poured into sat. NH₄Cl (50 mL) and extracted with ethyl acetate (3×50 mL), and the combined organic layers were dried with Na₂SO₄ and concentrated in vacuum. The residue was purified by column chromatography (SiO₂, 0 to 50% petroleum ether in ethyl acetate) to give tert-butyl 3-(2-(trifluoromethyl)pyridin-4-yl)azetidine-1-carboxylate. ¹H NMR (400 MHz, d₆-DMSO) δ 8.72 (d, J=5.0 Hz, 1H), 7.85 (s, 1H), 7.72 (d, J=4.8 Hz, 1H), 4.31-4.20 (m, 2H), 4.02-3.86 (m, 3H), 1.39 (s, 9H).

### Step 2:

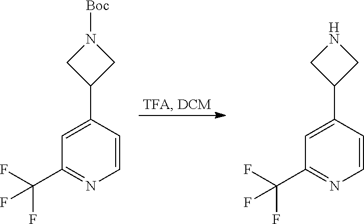

To a solution of tert-butyl 3-(2-(trifluoromethyl)pyridin-4-yl)azetidine-1-carboxylate (1.00 g, 3.31 mmol, 1.00 eq) in DCM (10 mL), was added TFA (4.62 g, 40.52 mmol, 3 mL, 12.25 eq). The reaction mixture was stirred at 25° C. for 2 h. The reaction mixture was concentrated in vacuum and dissolved in H₂O (50 mL), then extracted with DCM (3×10 mL), and the aqueous phase was lyophilized to give 4-(azetidin-3-yl)-2-(trifluoromethyl)pyridine, which was used without purification. ¹H NMR (400 MHz, d₆-DMSO) δ 8.77 (d, J=5.0 Hz, 1H), 7.99 (br d, J=0.9 Hz, 1H), 7.80-7.71 (m, 1H), 4.36-4.24 (m, 3H), 4.23-4.12 (m, 2H).

### Step 3:

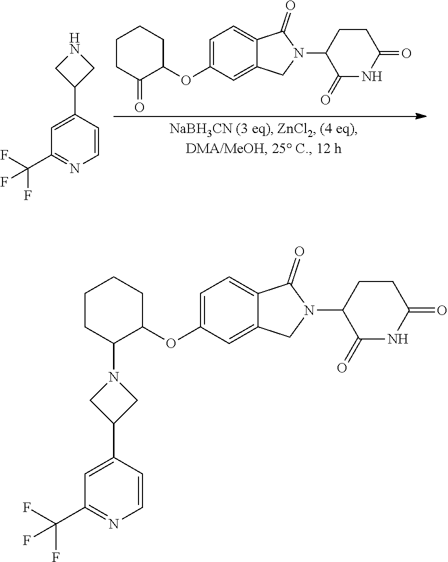

To a solution of 3-(1-oxo-5-((2-oxocyclohexyl)oxy)isoindolin-2-yl)piperidine-2,6-dione (80.00 mg, 224.48 μmol, 1.00 eq) and 4-(azetidin-3-yl)-2-(trifluoromethyl)pyridine (90.77 mg, 448.97 μmol, 2.00 eq) in DMA (1 mL) and MeOH (1 mL), was added ZnCl₂ (122.39 mg, 897.94 μmol, 42.06 μL, 4.00 eq). The reaction mixture was stirred at 25° C. for 10 h. NaBH₃CN (42.32 mg, 673.45 μmol, 3.00 eq) was added. The reaction mixture was stirred at 25° C. for 2 h. The reaction mixture was filtered, and the filtrate was concentrated to give the crude product. The residue was purified by prep-HPLC (method A) to give 3-(1-oxo-5-((2-(3-(2-(trifluoromethyl)pyridin-4-yl)azetidin-1-yl)cyclohexyl)oxy)isoindolin-2-yl)piperidine-2,6-dione. ¹H NMR (400 MHz, d₆-DMSO) δ 10.98 (s, 1H), 8.83-8.75 (m, 1H), 8.06-7.97 (m, 1H), 7.81-7.66 (m, 2H), 7.33-7.26 (m, 1H), 7.24-7.16 (m, 1H), 5.15-4.91 (m, 2H), 4.60-4.13 (m, 7H), 3.86-3.69 (m, 1H), 2.97-2.86 (m, 1H), 2.62 (br d, J=1.4 Hz, 1H), 2.39 (br dd, J=4.5, 13.0 Hz, 1H), 2.11-1.95 (m, 2H), 1.93-1.77 (m, 2H), 1.71-1.59 (m, 1H), 1.51-1.30 (m, 4H). LCMS (ESI): m/z 543.0 (M+H)⁺.

### Example 2-B (Method 2-B)

### Preparation of 3-(1-oxo-5-((2-(3-(pyrazin-2-yl)azetidin-1-yl)cyclohexyl)oxy)isoindolin-2-yl)piperidine-2,6-dione (Compound 2-B)

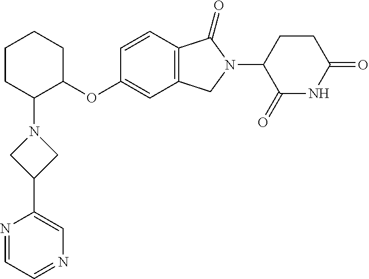 [SMILES: O=C1CCC(N2Cc3cc(OC4CCCCC4N4CC(c5cnccn5)C4)ccc3C2=O)C(=O)N1]

### Step 1:

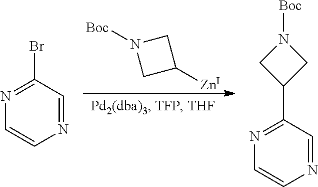

To a mixture of 2-bromopyrazine (1 g, 6.29 mmol, 1 eq) and (1-tert-butoxycarbonylazetidin-3-yl)-iodo-zinc (2.19 g, 6.29 mmol, 1 eq) in THF (30 mL), was added Pd₂(dba)₃ (287.99 mg, 314.50 μmol, 0.05 eq) and TFP (146.03 mg, 628.99 μmol, 0.1 eq) at 25° C. under a N₂ atmosphere. The mixture was stirred at 25° C. for 16 hrs. The mixture was poured into ice-water (100 mL) and stirred for 10 min. The aqueous phase was extracted with ethyl acetate (3×30 mL). The combined organic phase was washed with brine (2×20 mL), dried with anhydrous Na₂SO₄, filtered, and concentrated in vacuum. The residue was purified by silica gel chromatography (0 to 100% ethyl acetate in petroleum ether) to afford tert-butyl 3-(pyrazin-2-yl)azetidine-1-carboxylate. m/z 180.0 (M−56+H)⁺.

### Step 2:

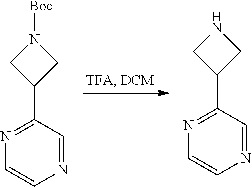 [SMILES: CC(C)(C)OC(=O)N1CC(c2cnccn2)C1 ; c1cnc(C2CNC2)cn1]

To a solution of tert-butyl 3-(pyrazin-2-yl)azetidine-1-carboxylate (1 g, 4.25 mmol, 1 eq) in DCM (9 mL), was added TFA (4.62 g, 40.52 mmol, 3 mL, 9.53 eq.) at 25° C. The mixture was stirred at 25° C. for 2 hrs. The mixture was concentrated in vacuum to afford 2-(azetidin-3-yl)pyrazine. The crude product was used in the next step without further purification. ¹H NMR (400 MHz, d₆-DMSO) δ 8.73 (s, 2H), 8.66-8.60 (m, 2H), 4.38-4.13 (m, 5H).

### Step 3:

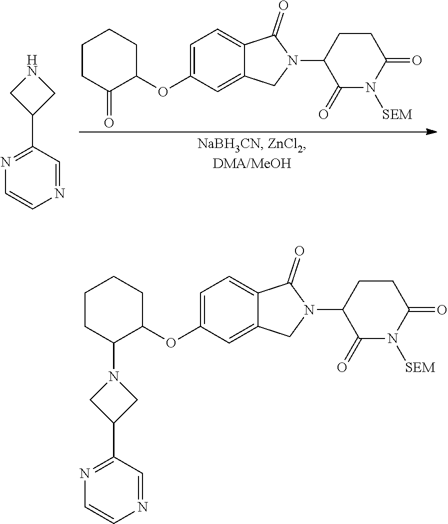

To a solution of 3-[1-oxo-5-(2-oxocyclohexoxy)isoindolin-2-yl]-1-(2-trimethylsilylethoxymethyl)piperidine-2,6-dione (300 mg, 616.48 μmol, 1 eq) and 2-(azetidin-3-yl)pyrazine (184.35 mg, 739.78 μmol, 1.2 eq) in DMA (2 mL) and MeOH (2 mL), was added ZnCl₂ (184.86 mg, 1.36 mmol, 63.53 μL, 2.2 eq) at 20° C. The reaction mixture was stirred at 20° C. for 2 hrs. NaBH₃CN was added (116.22 mg, 1.85 mmol, 3 eq). The reaction mixture was stirred at 20° C. for 3 hrs. Water (10 mL) was added, and the reaction stirred for 0.5 hr. The reaction mixture was extracted with EtOAc (3×30 mL). The combined organic layers were washed with brine (2×10 mL), dried over Na₂SO₄, filtered, and concentrated in vacuo to give 3-(1-oxo-5-((2-(3-(pyrazin-2-yl)azetidin-1-yl)cyclohexyl)oxy)isoindolin-2-yl)-1-((2-(trimethylsilyl)ethoxy)methyl)piperidine-2,6-dione. The crude product was used in the next step without further purification. m/z (ESI⁺): 606.4 (M+H)⁺.

### Step 4:

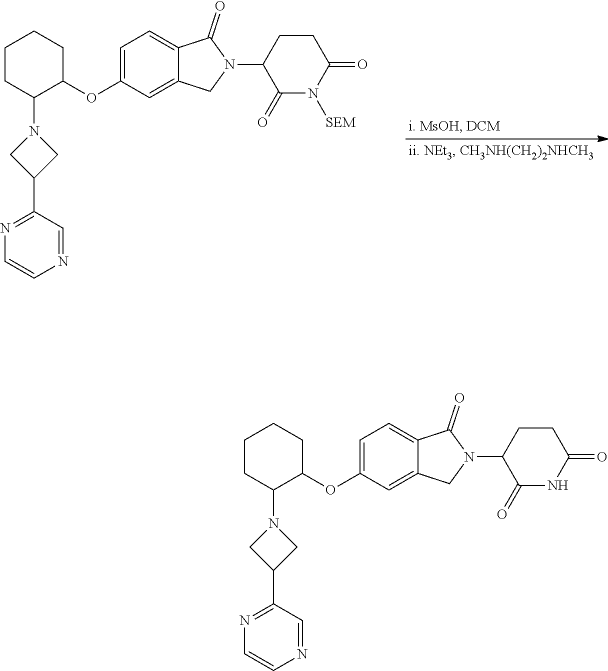 [SMILES: C[Si](C)(C)CCOCN1C(=O)CCC(N2Cc3cc(OC4CCCCC4N4CC(c5cnccn5)C4)ccc3C2=O)C1=O ; O=C1CCC(N2Cc3cc(OC4CCCCC4N4CC(c5cnccn5)C4)ccc3C2=O)C(=O)N1]

To a solution of 3-(1-oxo-5-((2-(3-(pyrazin-2-yl)azetidin-1-yl)cyclohexyl)oxy)isoindolin-2-yl)-1-((2-(trimethylsilyl)ethoxy)methyl)piperidine-2,6-dione (160 mg, 264.11 μmol, 1 eq) in DCM (5 mL), was added MsOH (101.53 mg, 1.06 mmol, 75.21 μL, 4 eq). The reaction mixture was stirred at 20° C. for 2 hrs. Triethylamine (213.80 mg, 2.11 mmol, 294.09 μL, 8 eq) and N,N′-dimethylethane-1,2-diamine (27.94 mg, 316.94 μmol, 34.11 μL, 1.2 eq) were added. The reaction mixture was stirred at 20° C. for 3 hrs. The mixture was concentrated in vacuo. The residue was purified by pre-HPLC (method A) to give 3-(1-oxo-5-((2-(3-(pyrazin-2-yl)azetidin-1-yl)cyclohexyl)oxy)isoindolin-2-yl)piperidine-2,6-dione. m/z (ESI⁺): 476.1 (M+H)⁺. ¹H NMR (400 MHz, d₆-DMSO) δ 10.98 (br s, 1H), 10.65-9.96 (m, 1H), 8.73 (br d, J=1.3 Hz, 1H), 8.70-8.60 (m, 2H), 7.68 (dd, J=8.5, 15.5 Hz, 1H), 7.42-7.07 (m, 2H), 5.14-5.03 (m, 1H), 4.91-4.16 (m, 7H), 3.87-3.57 (m, 1H), 3.00-2.82 (m, 1H), 2.59 (br d, J=17.4 Hz, 1H), 2.48-2.34 (m, 2H), 2.26-1.89 (m, 3H), 1.85-1.61 (m, 2H), 1.55-1.11 (m, 4H).

### Example 3-B (Method 3-B)

### Preparation of 3-(1-oxo-5-(((1S,2S)-2-(3-(quinolin-7-yl)azetidin-1-yl)cyclohexyl)oxy)isoindolin-2-yl)piperidine-2,6-dione (Compound 87-B)

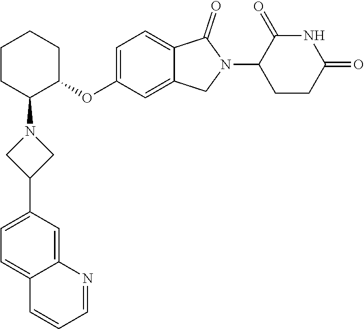 [SMILES: O=C1CCC(N2Cc3cc(O[C@H]4CCCC[C@@H]4N4CC(c5ccc6cccnc6c5)C4)ccc3C2=O)C(=O)N1]

### Step 1:

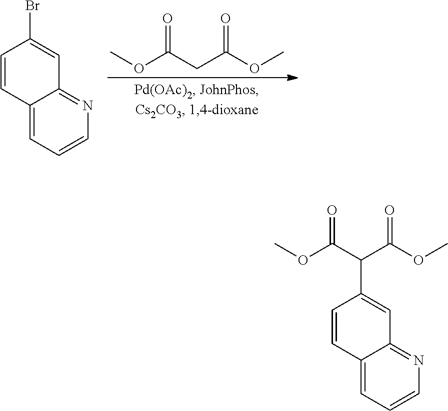

To a mixture of 7-bromoquinoline (2.00 g, 9.61 mmol, 1 eq) and dimethyl malonate (1.90 g, 14.42 mmol, 1.66 mL, 1.5 eq) in dioxane (20 mL), was added Cs₂CO₃ (9.40 g, 28.84 mmol, 3 eq). The reaction mixture was degassed and purged with N₂, and then JohnPhos (573.70 mg, 1.92 mmol, 0.2 eq) and Pd(OAc)₂ (215.82 mg, 961.29 μmol, 0.1 eq) was added. The mixture was stirred at 100° C. for 12 h. The mixture was filtered and concentrated under reduced pressure. The residue was purified by prep-HPLC (method A) to give dimethyl 2-(quinolin-7-yl)malonate. ¹H NMR (400 MHz, d₆-DMSO) δ 9.01 (dd, J=1.5, 4.4 Hz, 1H), 8.54 (br d, J=8.0 Hz, 1H), 8.16-8.02 (m, 2H), 7.80-7.60 (m, 2H), 5.40 (s, 1H), 3.72 (s, 6H).

### Step 2:

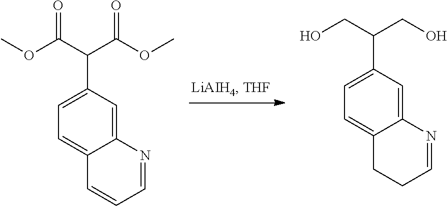

To a mixture of dimethyl 2-(quinolin-7-yl)malonate (1 g, 3.86 mmol, 1 eq) in THF (10 mL) was added LiAlH₄ (439.19 mg, 11.57 mmol, 3 eq) at 0° C. The mixture was stirred at 0° C. for 30 min. Then the mixture was stirred at 25° C. for 15.5 h. The mixture was added to Na₂SO₄·10H₂O (5.00 g) in THF (20.00 mL) at 0° C. and stirred at 0° C. for 10 min. The mixture was filtered and the filtrate was concentrated in vacuum to give 2-(3,4-dihydroquinolin-7-yl)propane-1,3-diol. m/z (ESI⁺) 206.1 (M+H)⁺.

### Step 3:

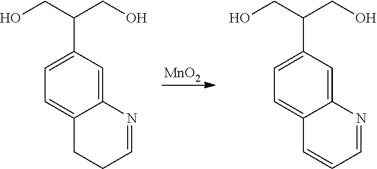

To a mixture of 2-(3,4-dihydroquinolin-7-yl)propane-1,3-diol (800.00 mg, 3.90 mmol, 1 eq) in DCM (10 mL) was added MnO₂ (1.36 g, 15.59 mmol, 4 eq) at 0° C. The mixture was stirred at 0° C. for 30 min. Then the mixture was stirred at 25° C. for 15.5 h. The mixture was filtered and the filtrate was concentrated in vacuum to give 2-(quinolin-7-yl)propane-1,3-diol. m/z (ESI⁺) 204.0 (M+H)⁺.

### Step 4:

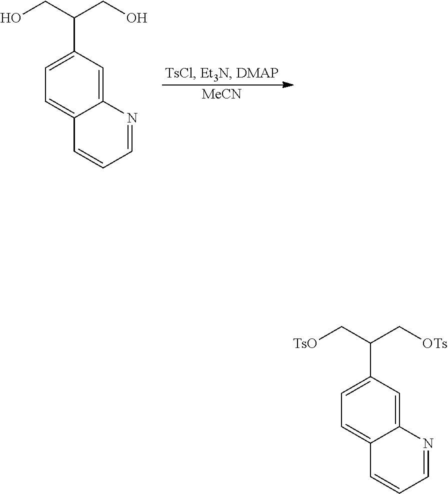

To a mixture of 2-(quinolin-7-yl)propane-1,3-diol (500 mg, 2.46 mmol, 1 eq) in ACN (20 mL) was added 4-methylbenzenesulfonyl chloride (1.64 g, 8.61 mmol, 3.5 eq), DMAP (30.06 mg, 246.02 μmol, 0.1 eq) and TEA (995.78 mg, 9.84 mmol, 1.37 mL, 4 eq) at 0° C. The mixture was stirred at 25° C. for 16 h. The mixture was added to ice-water (30 mL) at 0° C. and stirred at 0° C. for 10 min. The aqueous phase was extracted with ethyl acetate (3×20 mL). The combined organic phase was washed with brine (2×10 mL), dried with anhydrous Na₂SO₄, filtered, and concentrated in vacuum. The residue was purified by silica gel chromatography (0 to 100% ethyl acetate in petroleum ether) to give 2-(quinolin-7-yl)propane-1,3-diyl bis(4-methylbenzenesulfonate). ¹H NMR (400 MHz, d₆-DMSO) δ 8.95-8.86 (m, 1H), 8.39-8.31 (m, 1H), 7.82 (br d, J=8.5 Hz, 1H), 7.71 (s, 1H), 7.63-7.49 (m, 5H), 7.43-7.21 (m, 5H), 4.40-4.27 (m, 4H), 3.60-3.52 (m, 1H), 2.34 (s, 6H).

### Step 5:

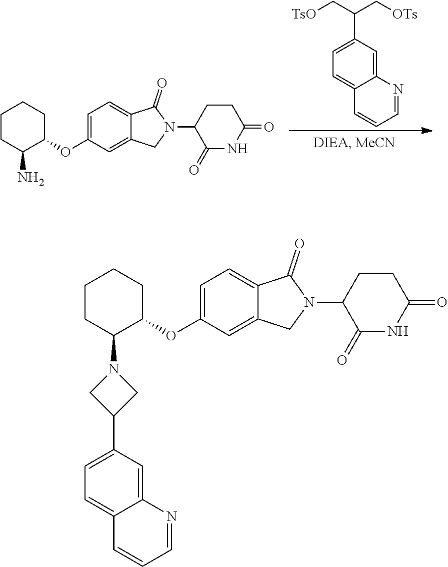 [SMILES: N[C@H]1CCCC[C@@H]1Oc1ccc2c(c1)CN(C1CCC(=O)NC1=O)C2=O ; Cc1ccc(S(=O)(=O)OCC(COS(=O)(=O)c2ccc(C)cc2)c2ccc3cccnc3c2)cc1 ; O=C1CCC(N2Cc3cc(O[C@H]4CCCC[C@@H]4N4CC(c5ccc6cccnc6c5)C4)ccc3C2=O)C(=O)N1]

To a solution of 3-(5-(((1S,2S)-2-aminocyclohexyl)oxy)-1-oxoisoindolin-2-yl)piperidine-2,6-dione prepared according to reported literature procedure [ADCOCK, Claire et al., US2020/17461, 2020, A1](80 mg, 223.84 μmol, 1 eq) and [3-(p-tolylsulfonyloxy)-2-(7-quinolyl)propyl]4-methylbenzenesulfonate (171.78 mg, 335.76 μmol, 1.5 eq) in ACN (5 mL) was added DIEA (115.72 mg, 895.35 μmol, 155.95 μL, 4 eq) at 25° C. The reaction mixture was stirred at 120° C. for 16 h. The mixture was filtered and filtrate was concentrated in vacuum. Then the crude product was purified by SFC (column: DAICEL CHIRALPAK AD (250 mm×30 mm, 10 um); mobile phase: IPA (0.1% IPAm); B %: 54%-54%, 12 min) to give 3-(1-oxo-5-(((1S,2S)-2-(3-(quinolin-7-yl)azetidin-1-yl)cyclohexyl)oxy)isoindolin-2-yl)piperidine-2,6-dione. ¹H NMR (400 MHz, d₆-DMSO-d6) δ 10.97 (s, 1H), 8.87 (br d, J=3.0 Hz, 1H), 8.32 (br d, J=8.3 Hz, 1H), 8.00-7.82 (m, 2H), 7.62 (br dd, J=8.4, 12.1 Hz, 2H), 7.47 (dd, J=4.1, 8.3 Hz, 1H), 7.21 (br s, 1H), 7.14-7.01 (m, 1H), 5.06 (dd, J=5.1, 13.3 Hz, 1H), 4.45-4.19 (m, 4H), 3.80-3.63 (m, 3H), 3.38 (br s, 1H), 3.30-3.24 (m, 1H), 2.96-2.84 (m, 1H), 2.58 (br d, J=17.1 Hz, 1H), 2.44-2.35 (m, 1H), 2.08-1.83 (m, 3H), 1.67 (br s, 2H), 1.46-1.35 (m, 2H), 1.30-1.13 (m, 2H).

### Example 4-B (Method 4-B)

### Preparation of 3-(1-oxo-5-(((1S,2S)-2-(3-(pyridin-4-yl)azetidin-1-yl)cyclohexyl)oxy)isoindolin-2-yl)piperidine-2,6-dione (Compound 85-B)

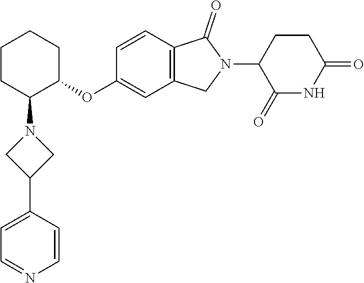 [SMILES: O=C1CCC(N2Cc3cc(O[C@H]4CCCC[C@@H]4N4CC(c5ccncc5)C4)ccc3C2=O)C(=O)N1]

### Step 1:

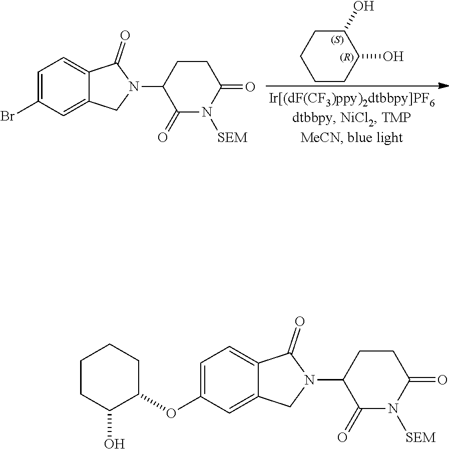

To a mixture of 3-(5-bromo-1-oxoisoindolin-2-yl)-1-((2-(trimethylsilyl)ethoxy)methyl)piperidine-2,6-dione (3.84 g, 33.08 mmol, 1.5 eq) [prepared according to literature procedure described in PCT Int. Appl. WO2020012334], (1R,2S)-cyclohexane-1,2-diol (2.55 g, 22.0 mmol, 1.0 eq), dtbbpy (295.98 mg, 1.10 mmol, 0.05 eq), Ir[(dF(CF₃)ppy)₂dtbbpy]PF₆ (247.44 mg, 220.56 μmol, 0.01 eq), and NiCl₂·glyme (242.30 mg, 1.10 mmol, 0.05 eq) in CH₃CN (100 mL), was added TMP (3.74 g, 26.47 mmol, 4.49 mL, 1.2 eq). The reaction mixture was stirred at 2-25° C. (e.g., 2 5° C., 2° C., 5° C., 10° C., or 25° C.), for 12 hrs. The reaction mixture was filtered and then concentrated in vacuum. The residue was purified by column chromatography (50 to 100% ethyl acetate in petroleum ether) to give 3-(5-(((1S,2R)-2-hydroxycyclohexyl)oxy)-1-oxoisoindolin-2-yl)-1-((2-(trimethylsilyl)ethoxy)methyl)piperidine-2,6-dione. ¹H NMR (400 MHz, d₆-DMSO) δ 7.65-7.57 (m, 1H), 7.19 (s, 1H), 7.07 (dd, J=2.1, 8.4 Hz, 1H), 5.18 (dd, J=5.0, 13.4 Hz, 1H), 5.05 (q, J=9.7 Hz, 2H), 4.94 (dd, J=1.2, 4.7 Hz, 1H), 4.47 (d, J=3.6 Hz, 1H), 4.40 (dd, J=4.9, 17.1 Hz, 1H), 4.26-4.12 (m, 2H), 3.61-3.46 (m, 3H), 3.14-2.99 (m, 2H), 2.78 (br dd, J=2.1, 15.6 Hz, 1H), 2.43-2.28 (m, 1H), 2.07-2.01 (m, 2H), 1.94-1.82 (m, 1H), 1.79-1.69 (m, 1H), 1.63 (br d, J=9.6 Hz, 2H), 1.58-1.53 (m, 1H), 1.37-1.27 (m, 3H), 1.13 (br d, J=7.9 Hz, 1H), 0.90-0.78 (m, 2H), 0.02 (s, 9H).

### Step 2:

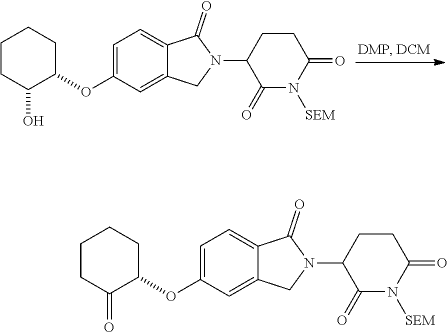

To a mixture of 3-(5-(((1S,2R)-2-hydroxycyclohexyl)oxy)-1-oxoisoindolin-2-yl)-1-((2-(trimethylsilyl)ethoxy)methyl)piperidine-2,6-dione (5 g, 10.23 mmol, 1 eq) in DCM (50 mL), was added DMP (8.68 g, 20.46 mmol, 6.34 mL, 2 eq). The mixture was stirred at 25° C. for 2 hrs. The reaction mixture was filtered and concentrated in vacuum. The residue was purified by column chromatography (50 to 100% ethyl acetate in petroleum ether) to give 3-(1-oxo-5-(((S)-2-oxocyclohexyl)oxy)isoindolin-2-yl)-1-((2-(trimethylsilyl)ethoxy)methyl)piperidine-2,6-dione. ¹H NMR (400 MHz, d₆-DMSO) δ 7.59 (d, J=8.4 Hz, 1H), 7.07 (s, 1H), 7.00 (dd, J=1.2, 8.4 Hz, 1H), 5.25-5.14 (m, 2H), 5.09-4.97 (m, 2H), 4.38 (dd, J=5.0, 17.0 Hz, 1H), 4.25-4.15 (m, 1H), 3.72-3.42 (m, 2H), 3.16-3.00 (m, 1H), 2.87-2.73 (m, 1H), 2.71-2.58 (m, 1H), 2.40-2.28 (m, 3H), 2.10-1.99 (m, 2H), 1.93-1.74 (m, 3H), 1.66-1.51 (m, 1H), 0.88-0.79 (m, 2H), 0.02 (d, J=1.4 Hz, 9H).

### Step 3:

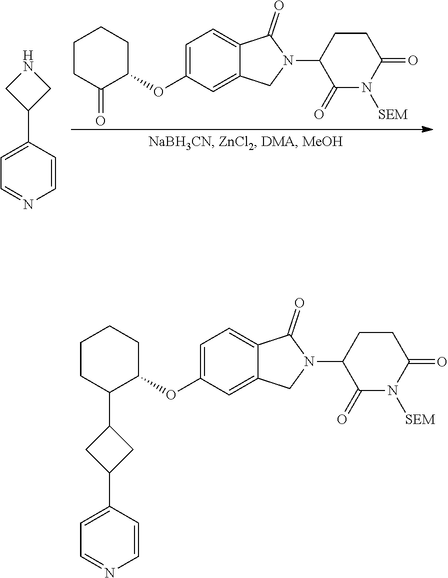

To a solution of 3-(1-oxo-5-(((S)-2-oxocyclohexyl)oxy)isoindolin-2-yl)-1-((2-(trimethylsilyl)ethoxy)methyl)piperidine-2,6-dione (300 mg, 616.48 μmol, 1 eq) and 4-(azetidin-3-yl)pyridine (99 mg, 739.78 μmol, 1.2 eq) in DMA (2 mL) and MeOH (2 mL), was added ZnCl₂ (184.86 mg, 1.36 mmol, 63.53 μL, 2.2 eq) at 20° C. The reaction mixture was stirred at 20° C. for 2 hrs. NaBH₃CN was added (116.22 mg, 1.85 mmol, 3 eq). The reaction mixture was stirred at 20° C. for 5 hrs. Water (10 mL) was added, and the reaction stirred for 0.5 hr. The reaction mixture was extracted with EtOAc (3×30 mL). The combined organic layers were washed with brine (2×10 mL), dried over Na₂SO₄, filtered, and concentrated in vacuo to give 3-(1-oxo-5-(((1S)-2-(3-(pyridin-4-yl)azetidin-1-yl)cyclohexyl)oxy)isoindolin-2-yl)-1-((2-(trimethylsilyl)ethoxy)methyl)piperidine-2,6-dione. The crude product was used in the next step without further purification.

### Step 4:

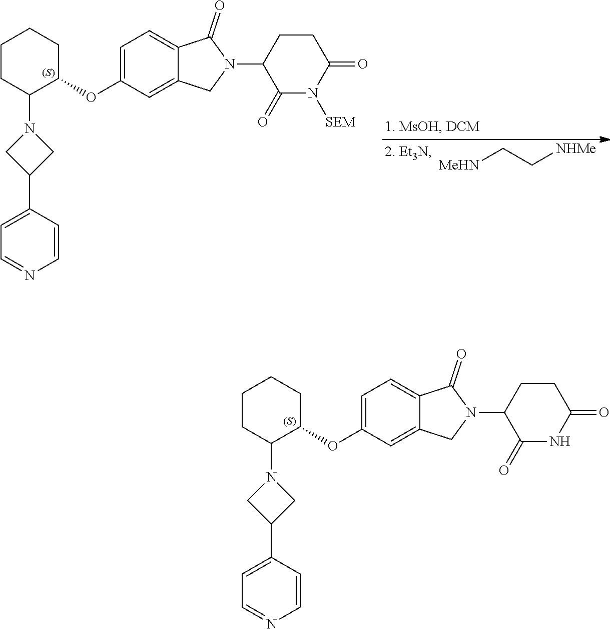

To a solution of 3-(1-oxo-5-(((1S)-2-(3-(pyridin-4-yl)azetidin-1-yl)cyclohexyl)oxy)isoindolin-2-yl)-1-((2-(trimethylsilyl)ethoxy)methyl)piperidine-2,6-dione (400 mg, 661.36 μmol, 1 eq) in DCM (10 mL) was added MsOH (254.26 mg, 2.65 mmol, 188.34 μL) at 25° C. The reaction mixture was stirred at 25° C. for 4 h. TEA (535.39 mg, 5.29 mmol, 736.43 μL) and N,N′-dimethylethane-1,2-diamine (69.96 mg, 793.64 μmol, 85.42 uL, 1.2 eq) was added and the reaction was stirred at 25° C. for 12 h. The mixture was filtered and the filtrate was concentrated in vacuum to give 3-(1-oxo-5-(((1S)-2-(3-(pyridin-4-yl)azetidin-1-yl)cyclohexyl)oxy)isoindolin-2-yl)piperidine-2,6-dione.

### Step 5:

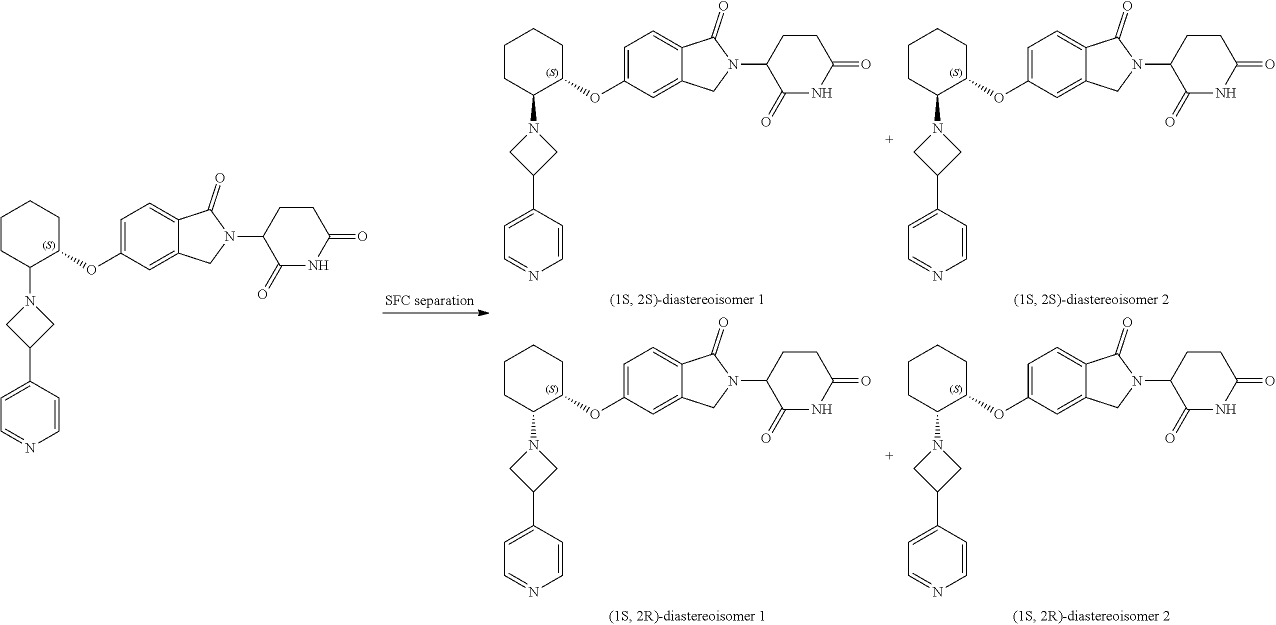

The residue was separation by SFC (column: DAICEL CHIRALPAK AD (250 mm×30 mm, 10 μm); mobile phase: IPA (0.1% IPAm); B %: 54%-54%, 13 min) to give 3-(1-oxo-5-(((1S,2S)-2-(3-(pyridin-4-yl)azetidin-1-yl)cyclohexyl)oxy)isoindolin-2-yl)piperidine-2,6-dione (diastereoisomer 1), 3-(1-oxo-5-(((1 S,2S)-2-(3-(pyridin-4-yl)azetidin-yl)cyclohexyl)oxy)isoindolin-2-yl)piperidine-2,6-dione dione (diastereoisomer 2), 3-(1-oxo-5-(((1S,2R)-2-(3-(pyridin-4-yl)azetidin-1-yl)cyclohexyl)oxy)isoindolin-2-yl)piperidine-2,6-dione (diastereoisomer 1), and 3-(1-oxo-5-(((1S,2R)-2-(3-(pyridin-4-yl)azetidin-1-yl)cyclohexyl)oxy)isoindolin-2-yl)piperidine-2,6-dione (diastereoisomer 2).

Additional compounds can also be prepared following the procedures set forth above, with the exception that the amines or carboxylic acids in the above examples are replaced with an amine or carboxylic acid depicted in the final product.

TABLE 2

| # | ¹H NMR | Method |
|---|---|---|
| 12-A | ¹H NMR (400 MHz, d₄-Methanol) | |
| | δ 0.90-1.14 (m, 2H), 1.26-1.53 (m, 4H), | |
| | 1.69 (br t, J = 14.76 Hz, 2H), 1.76-1.92 (m, | |
| | 3H), 2.07 (s, 3H), 2.16 (ddt, J = 9.97, 5.13, | |
| | 2.58 Hz, 2H), 2.27 (br d, J = 11.01 Hz, 1H), | |
| | 2.41-2.65 (m, 3H), 2.74-2.83 (m, 1H), | |
| | 2.85-2.97 (m, 1H), 3.02-3.14 (m, 1H), | |
| | 3.79-4.16 (m, 5H), 4.38-4.58 (m, 4H), | |
| | 5.12 (dd, J = 13.38, 5.13 Hz, 1H), 7.09-7.27 | |
| | (m, 2H), 7.74 (d, J = 8.38 Hz, 1H). | |
| 14-A | ¹H NMR (400 MHz, d₆-DMSO) δ 1.13-1.53 | |
| | (m, 5H), 1.66-1.82 (m, 2H), 1.86-2.25 (m, | |
| | 5H), 2.56-2.64 (m, 1H), 2.64-2.76 (m, 1H), | |
| | 2.85-2.97 (m, 1H), 3.36 (br s, 2H), 3.51-3.88 | |
| | (m, 6H), 4.03 (br d, J = 6.40 Hz, 2H), 4.23- | |
| | 4.53 (m, 3H), 5.08 (dd, J = 13.18, 5.02 Hz, | |
| | 1H), 7.14 (br dd, J = 6.15, 1.63 Hz, 1H), 7.23- | |
| | 7.30 (m, 1H), 7.67 (d, J = 8.28 Hz, 1H), | |
| | 10.16-10.33 (m, 1H), 10.97 (s, 1H). | |
| 24-A | ¹H NMR (400 MHz, d₄-Methanol) | |
| | δ 0.95-1.17 (m, 3H), 1.26-1.47 (m, 3H), | |
| | 1.58 (br d, J = 10.88 Hz, 1H), 1.65-1.81 (m, | |
| | 4H), 1.96 (br d, J = 10.63 Hz, 1H), 2.09- | |
| | 2.26 (m, 3H), 2.39-2.53 (m, 2H), 2.73- | |
| | 2.95 (m, 3H), 2.96-3.12 (m, 2H), 3.20 (br s, | |
| | 1H), 3.52 (br s, 2H), 3.70 (br d, J = 13.13 | |
| | Hz, 1H), 4.22-4.30 (m, 1H), 4.36-4.50 (m, | |
| | 2H), 4.60 (br d, J = 12.76 Hz, 1H), 5.11 (dd, | |
| | J = 13.26, 5.13 Hz, 1H), 7.00-7.16 (m, 2H), | |
| | 7.33-7.40 (m, 2H), 7.40-7.49 (m, 3H), | |
| | 7.70 (br d, J = 8.38 Hz, 1H) | |
| 25-A | ¹H NMR (400 MHz, d₄-Methanol) | |
| | δ 0.90-1.09 (m, 2H), 1.16-1.30 (m, 1H), | |
| | 1.33-1.48 (m, 6H), 1.63-1.72 (m, 3H), | |
| | 1.72-1.78 (m, 1H), 1.78-1.92 (m, 4H), | |
| | 1.92-2.04 (m, 1H), 2.10 (br d, J = 11.13 Hz, | |
| | 1H), 2.16 (dtd, J = 12.69, 5.25, 2.44 Hz, 1H), | |
| | 2.23 (br d, J = 10.26 Hz, 1H), 2.34-2.53 (m, | |
| | 4H), 2.53-2.63 (m, 1H), 2.74-2.82 (m, | |
| | 1H), 2.85-2.93 (m, 1H), 2.93-3.08 (m, | |
| | 2H), 3.54 (br s, 1H), 3.63-3.77 (m, 2H), | |
| | 3.89 (br d, J = 7.25 Hz, 2H), 4.35-4.52 (m, | |
| | 4H), 5.12 (dd, J = 13.26, 5.00 Hz, 1H), 7.10 | |
| | (br d, J = 8.38 Hz, 1H), 7.18 (s, 1H), 7.73 (d, | |
| | J = 8.38 Hz, 1H) | |
| 26-A | ¹H NMR (400 MHz, d₄-Methanol) | |
| | δ 0.87-1.00 (m, 2 H), 1.03-1.15 (m, 1 H), | |
| | 1.27-1.42 (m, 4 H), 1.44 (s, 9 H), 1.53- | |
| | 1.64 (m, 3 H), 1.76 (br s, 2 H), 1.92-2.01 | |
| | (m, 1 H), 2.08-2.20 (m, 3 H), 2.41-2.54 | |
| | (m, 2 H), 2.64-2.76 (m, 2 H), 2.76-2.82 | |
| | (m, 1 H), 2.85-2.92 (m, 1 H), 2.96 (br dd, J = | |
| | 13.57, 5.94 Hz, 1 H), 3.15-3.22 (m, 1 H), | |
| | 3.46-3.56 (m, 2 H), 4.04 (br d, J = 13.13 | |
| | Hz, 2 H), 4.21-4.30 (m, 1 H), 4.37-4.50 | |
| | (m, 2 H), 5.11 (dd, J = 13.45, 5.19 Hz, 1 H), | |
| | 7.05 (dd, J = 8.50, 2.13 Hz, 1 H), 7.13 (s, 1 | |
| | H), 7.70 (d, J = 8.38 Hz, 1 H). | |
| 27-A | ¹H NMR (400 MHz, d₄-Methanol) | |
| | δ 0.95-1.60 (m, 10H), 1.69-1.91 (m, 5H), | |
| | 2.10-2.31 (m, 3H), 2.48 (qd, J = 12.92, 4.50 | |
| | Hz, 2H), 2.62-2.83 (m, 2H), 2.84-2.96 (m, | |
| | 1H), 3.09-3.28 (m, 2H), 3.68-3.78 (m, | |
| | 1H), 3.91 (br s, 1H), 4.02 (br d, J = 7.50 Hz, | |
| | 2H), 4.33-4.52 (m, 5H), 5.12 (dd, J = 13.26, | |
| | 5.00 Hz, 1H), 7.12 (br d, J = 8.38 Hz, 1H), | |
| | 7.21 (br s, 1H), 7.74 (d, J = 8.38 Hz, 1H). | |
| 28-A | ¹H NMR (400 MHz, d₄-Methanol) | |
| | δ 0.88-1.06 (m, 2H), 1.17 (s, 3H), 1.21- | |
| | 1.52 (m, 4H), 1.61-1.73 (m, 2H), 1.75- | |
| | 1.89 (m, 3H), 1.93-2.03 (m, 6H), 2.07- | |
| | 2.28 (m, 3H), 2.46 (td, J = 13.10, 3.56 Hz, | |
| | 2H), 2.59 (br t, J = 12.26 Hz, 1H), 2.72- | |
| | 2.82 (m, 1H), 2.84-2.96 (m, 1H), 2.97- | |
| | 3.15 (m, 2H), 3.60 (br s, 1H), 3.79 (br s, 1H), | |
| | 3.93 (br s, 2H), 4.24 (br d, J = 12.38 Hz, | |
| | 1H), 4.45 (br s, 4H), 5.12 (br dd, J = 13.07, | |
| | 4.57 Hz, 1H), 7.05-7.22 (m, 2H), 7.72 (br d, | |
| | J = 8.38 Hz, 1H), 8.50 (s, 1H). | |
| 29-A | ¹H NMR (400 MHz, d₄-Methanol) | |
| | δ 0.94-1.11 (m, 2H), 1.20-1.50 (m, 4H), | |
| | 1.62 (s, 3H), 1.69 (br d, J = 12.52 Hz, 2H), | |
| | 1.75-1.90 (m, 3H), 2.09-2.21 (m, 2H), | |
| | 2.26 (br d, J = 10.13 Hz, 1H), 2.48 (qd, J = | |
| | 12.93, 4.59 Hz, 2H), 2.63 (br t, J = 12.46 Hz, | |
| | 1H), 2.73-2.82 (m, 1H), 2.85-2.96 (m, | |
| | 1H), 3.00-3.12 (m, 2H), 3.18 (br s, 1H), | |
| | 3.71 (br s, 1H), 3.88 (br s, 1H), 4.00 (br d, J = | |
| | 6.79 Hz, 2H), 4.33 (br d, J = 5.84 Hz, 2H), | |
| | 4.39-4.53 (m, 4H), 4.91 (br d, J = 5.13 Hz, | |
| | 2H), 5.12 (dd, J = 13.29, 5.07 Hz, 1H), 7.08- | |
| | 7.22 (m, 2H), 7.74 (d, J = 7.99 Hz, 1H), | |
| | 8.46 (br s, 1H). | |
| 30-A | ¹H NMR (400 MHz, d₄-Methanol) | |
| | δ 1.06 (q, J = 11.52 Hz, 1H), 1.13-1.27 (m, | |
| | 1H), 1.26-1.55 (m, 3H), 1.73 (br d, J = | |
| | 11.80 Hz, 1H), 1.81-2.02 (m, 3H), 2.08- | |
| | 2.27 (m, 5H), 2.28-2.36 (m, 1H), 2.41- | |
| | 2.96 (m, 12H), 3.14 (br t, J = 13.17 Hz, 1H), | |
| | 3.46-3.55 (m, 1H), 3.77 (br d, J = 13.35 Hz, | |
| | 1H), 4.05 (br t, J = 9.00 Hz, 1H), 4.22 (br d, | |
| | J = 5.60 Hz, 2H), 4.39-4.58 (m, 3H), 5.09- | |
| | 5.16 (m, 1H), 7.12-7.18 (m, 1H), 7.23 (s, | |
| | 1H), 7.75 (d, J = 8.34 Hz, 1H). | |
| 31-A | ¹H NMR (400 MHz, d₆-DMSO) δ 10.96 (s, | |
| | 1H), 8.15 (s, 1H), 7.61 (d, J = 8.4 Hz, 1H), | |
| | 7.17 (s, 1H), 7.03 (br d, J = 8.4 Hz, 1H), 5.06 | |
| | (dd, J = 5.1, 13.1 Hz, 1H), 4.42-4.33 (m, | |
| | 1H), 4.30-4.17 (m, 4H), 3.05 (br t, J = 6.9 | |
| | Hz, 2H), 2.96-2.86 (m, 2H), 2.73 (br dd, J = | |
| | 5.5, 9.4 Hz, 1H), 2.64-2.52 (m, 3H), 2.43- | |
| | 2.34 (m, 2H), 2.12-1.94 (m, 3H), 1.80 (br d, | |
| | J = 11.6 Hz, 1H), 1.70-1.54 (m, 5H), 1.35 | |
| | (br t, J = 8.3 Hz, 2H), 1.26-1.02 (m, 5H), | |
| | 0.85 (br d, J = 11.4 Hz, 2H), 0.76-0.69 (m, | |
| | 2H), 0.54-0.44 (m, 2H). | |
| 32-A | ¹H NMR (400 MHz, d₆-DMSO) | |
| | δ 10.99 (s, 1H), 8.15 (s, 1H), 7.62 (d, J = 8.4 | |
| | Hz, 1H), 7.18 (br s, 1H), 7.04 (br d, J = 8.4 | |
| | Hz, 1H), 5.07 (dd, J = 5.1, 13.2 Hz, 1H), | |
| | 4.44-4.19 (m, 4H), 3.70 (br d, J = 13.0 Hz, | |
| | 1H), 3.50-3.44 (m, 2H), 3.24-3.12 (m, | |
| | 3H), 3.08-2.98 (m, 1H), 2.96-2.83 (m, | |
| | 2H), 2.79-2.68 (m, 4H), 2.59 (br d, J = 18.9 | |
| | Hz, 2H), 2.42-2.35 (m, 1H), 2.14-1.92 (m, | |
| | 3H), 1.82 (br d, J = 7.8 Hz, 1H), 1.70-1.47 | |
| | (m, 5H), 1.40-1.04 (m, 4H), 0.94-0.74 (m, | |
| | 2H). | |
| 33-A | ¹H NMR (400 MHz, d₆-DMSO) | |
| | δ 10.99 (s, 1H), 8.14 (s, 1H), 7.62 (d, J = 8.3 | |
| | Hz, 1H), 7.18 (s, 1H), 7.04 (br d, J = 8.4 Hz, | |
| | 1H), 5.07 (dd, J = 4.9, 13.3 Hz, 1H), 4.43- | |
| | 4.33 (m, 1H), 4.33-4.21 (m, 3H), 4.20- | |
| | 4.04 (m, 2H), 3.52-3.42 (m, 2H), 3.05- | |
| | 2.94 (m, 1H), 2.93-2.85 (m, 1H), 2.58 (br d, | |
| | J = 19.9 Hz, 2H), 2.41-2.35 (m, 2H), 2.12- | |
| | 1.96 (m, 5H), 1.88-1.76 (m, 1H), 1.63 (br s, | |
| | 2H), 1.57 (br d, J = 14.4 Hz, 5H), 1.51-1.41 | |
| | (m, 5H), 1.39-1.30 (m, 2H), 1.22 (br d, J = | |
| | 6.0 Hz, 1H), 1.19-1.12 (m, 3H), 1.11-1.02 | |
| | (m, 1H), 0.90-0.78 (m, 2H). | |
| 34-A | ¹H NMR (400 MHz, d₆-DMSO) | |
| | δ 10.99 (s, 1H), 8.14 (s, 1H), 7.62 (d, J = 8.4 | |
| | Hz, 1H), 7.18 (s, 1H), 7.05 (br d, J = 8.4 Hz, | |
| | 1H), 5.07 (dd, J = 5.0, 13.3 Hz, 1H), 4.48- | |
| | 4.14 (m, 4H), 3.79 (br d, J = 13.5 Hz, 1H), | |
| | 3.55-3.41 (m, 3H), 3.13-3.02 (m, 1H), | |
| | 2.96-2.82 (m, 2H), 2.59 (br d, J = 17.0 Hz, | |
| | 2H), 2.46-2.35 (m, 3H), 2.20 (br d, J = 5.3 | |
| | Hz, 2H), 2.14-1.93 (m, 3H), 1.84 (br d, J = | |
| | 9.4 Hz, 1H), 1.65-1.50 (m, 4H), 1.41-1.30 | |
| | (m, 2H), 1.29-1.19 (m, 1H), 1.16-1.02 (m, | |
| | 1H), 0.96-0.73 (m, 3H), 0.46-0.37 (m, | |
| | 2H), 0.08 (q, J = 4.8 Hz, 2H). | |
| 35-A | ¹H NMR (400 MHz, d₆-DMSO) | |
| | δ 10.98 (s, 1H), 8.14 (s, 1H), 7.62 (br d, J = | |
| | 8.4 Hz, 1H), 7.18 (br s, 1H), 7.04 (br d, J = | |
| | 8.3 Hz, 1H), 5.07 (br dd, J = 4.8, 13.0 Hz, | |
| | 1H), 4.47-4.10 (m, 6H), 3.49-3.44 (m, | |
| | 3H), 3.19-3.12 (m, 1H), 3.07-2.82 (m, | |
| | 4H), 2.61 (br s, 3H), 2.38 (br d, J = 14.1 Hz, | |
| | 1H), 2.14-1.95 (m, 2H), 1.83 (br d, J = 7.5 | |
| | Hz, 1H), 1.71-1.51 (m, 5H), 1.41-1.19 (m, | |
| | 3H), 1.11-1.02 (m, 4H), 0.98-0.69 (m, | |
| | 3H), 0.57-0.42 (m, 1H). | |
| 41-A | ¹H NMR (400 MHz, d₄-Methanol) | |
| | δ 1.90-2.04 (m, 6 H) 2.12-2.21 (m, 1 H) | |
| | 2.31-2.42 (m, 2 H) 2.49 (br dd, J = 13.14, | |
| | 4.71 Hz, 1 H) 2.73-2.82 (m, 1 H) 2.84- | |
| | 2.91 (m, 1 H) 2.95 (s, 3 H) 2.98-3.08 (m, 1 | |
| | H) 3.16 (s, 3 H) 3.19-3.29 (m, 2 H) 3.39- | |
| | 3.55 (m, 4 H) 3.84 (br d, J = 12.47 Hz, 2 H) | |
| | 4.38 (s, 2 H) 4.43-4.58 (m, 2 H) 5.14 (dd, | |
| | J = 13.33, 5.01 Hz, 1 H) 7.36 (br d, J = 8.44 | |
| | Hz, 2 H) 7.58 (d, J = 8.56 Hz, 2 H) 7.65 (br d, | |
| | J = 7.95 Hz, 1 H) 7.72-7.83 (m, 2 H). | |
| 42-A | ¹H NMR (400 MHz, d₄-Methanol) | |
| | δ 0.98-1.54 (m, 10H), 1.65-1.94 (m, 5H), | |
| | 2.11-2.21 (m, 2H), 2.28 (br d, J = 10.76 Hz, | |
| | 1H), 2.40-2.59 (m, 2H), 2.61-2.83 (m, | |
| | 2H), 2.84-2.98 (m, 1H), 3.04-3.22 (m, | |
| | 1H), 3.35 (s, 1H), 3.85 (br t, J = 8.50 Hz, | |
| | 1H), 3.97-4.16 (m, 3H), 4.32-4.55 (m, | |
| | 5H), 5.12 (dd, J = 13.32, 5.07 Hz, 1H), 7.06- | |
| | 7.26 (m, 2H), 7.74 (d, J = 8.38 Hz, 1H), | |
| | 8.40-8.53 (m, 1H), 8.47 (br s, 1H). | |
| 55-A | ¹H NMR (400 MHz, d₆-DMSO) | |
| | δ 10.96 (s, 1H), 8.19 (s, 1H), 7.61 (d, J = 8.4 | |
| | Hz, 1H), 7.16 (s, 1H), 7.06-6.97 (m, 1H), | |
| | 5.06 (dd, J = 4.6, 13.2 Hz, 1H), 4.53 (br d, J = | |
| | 14.1 Hz, 1H), 4.44-4.33 (m, 1H), 4.25 (br | |
| | dd, J = 6.3, 16.9 Hz, 2H), 3.95-3.78 (m, | |
| | 2H), 3.44 (br dd, J = 4.3, 12.9 Hz, 1H), 3.37- | |
| | 3.28 (m, 4H), 3.07 (br t, J = 6.8 Hz, 1H), | |
| | 2.98 (br t, J = 7.0 Hz, 1H), 2.95-2.80 (m, | |
| | 2H), 2.59 (br d, J = 17.3 Hz, 1H), 2.43-2.31 | |
| | (m, 2H), 2.29-2.18 (m, 1H), 2.03-1.92 (m, | |
| | 2H), 1.87-1.70 (m, 2H), 1.63 (br s, 2H), | |
| | 1.40-1.31 (m, 3H), 1.23 (br d, J = 7.4 Hz, | |
| | 1H), 1.13-0.98 (m, 1H). | |
| 56-A | ¹H NMR (400 MHz, d₄-Methanol) | |
| | δ 1.25-1.55 (m, 7H), 1.77-2.02 (m, 7H), | |
| | 2.12-2.27 (m, 4H), 2.33 (br s, 3H), 2.41- | |
| | 2.55 (m, 1H), 2.66-2.78 (m, 2H), 2.79- | |
| | 2.83 (m, 1H), 2.86-2.96 (m, 1H), 3.47- | |
| | 3.55 (m, 2H), 3.58-3.66 (m, 1H), 4.03- | |
| | 4.34 (m, 4H), 4.39-4.59 (m, 4H), 5.13 (br | |
| | dd, J = 13.45, 4.57 Hz, 1H), 7.11-7.18 (m, | |
| | 1H), 7.24 (br s, 1H), 7.75 (br d, J = 7.50 Hz, | |
| | 1H). | |
| 57-A | ¹H NMR (400 MHz, d₄-Methanol) | |
| | δ 1.04-1.18 (m, 2H), 1.24-1.53 (m, 4H), | |
| | 1.60-1.74 (m, 3H), 1.79-1.92 (m, 2H), | |
| | 1.93-2.01 (m, 1H), 2.02-2.11 (m, 1H), | |
| | 2.11-2.21 (m, 2H), 2.22-2.33 (m, 3H), | |
| | 2.39-2.57 (m, 4H), 2.70-2.83 (m, 3H), | |
| | 2.85-2.96 (m, 1H), 3.35 (br s, 1H), 3.72 (br | |
| | d, J = 12.01 Hz, 2H), 3.84 (br d, J = 7.38 Hz, | |
| | 1H), 3.89-4.16 (m, 4H), 4.38-4.52 (m, | |
| | 3H), 5.08-5.18 (m, 1H), 7.13 (br d, J = 8.38 | |
| | Hz, 1H), 7.22 (br s, 1H), 7.75 (d, J = 8.38 | |
| | Hz, 1H). | |
| 1-B | (400 MHz, d₆-DMSO) δ 10.98 (s, 1H), 8.83- | 1-B |
| | 8.75 (m, 1H), 8.06-7.97 (m, 1H), 7.81- | |
| | 7.66 (m, 2H), 7.33-7.26 (m, 1H), 7.24- | |
| | 7.16 (m, 1H), 5.15-4.91 (m, 2H), 4.60- | |
| | 4.13 (m, 7H), 3.86-3.69 (m, 1H), 2.97- | |
| | 2.86 (m, 1H), 2.62 (br d, J = 1.4 Hz, 1H), | |
| | 2.39 (br dd, J = 4.5, 13.0 Hz, 1H), 2.11-1.95 | |
| | (m, 2H), 1.93-1.77 (m, 2H), 1.71-1.59 (m, | |
| | 1H), 1.51-1.30 (m, 4H). | |
| 2-B | (400 MHz, d₆-DMSO) δ 10.98 (br s, 1H), | 2-B |
| | 10.65-9.96 (m, 1H), 8.73 (br d, J = 1.3 Hz, | |
| | 1H), 8.70-8.60 (m, 2H), 7.68 (dd, J = 8.5, | |
| | 15.5 Hz, 1H), 7.42-7.07 (m, 2H), 5.14-5.03 | |
| | (m, 1H), 4.91-4.16 (m, 7H), 3.87-3.57 (m, | |
| | 1H), 3.00-2.82 (m, 1H), 2.59 (br d, J = 17.4 | |
| | Hz, 1H), 2.48-2.34 (m, 2H), 2.26-1.89 (m, | |
| | 3H), 1.85-1.61 (m, 2H), 1.55-1.11 (m, 4H) | |
| 6-B | (400 MHz, d₆-DMSO) δ 10.99 (s, 1H), 10.71- | 1-B |
| | 10.37 (m, 1H), 9.03-8.87 (m, 2H), 7.76- | |
| | 7.62 (m, 1H), 7.36-7.23 (m, 1H), 7.13 (br | |
| | dd, J = 2.0, 8.3 Hz, 1H), 5.17-4.87 (m, 2H), | |
| | 4.83-4.72 (m, 1H), 4.69-4.20 (m, 7H), 3.82- | |
| | 3.57 (m, 2H), 3.01-2.84 (m, 1H), 2.28- | |
| | 2.08 (m, 2H), 1.98 (td, J = 2.6, 9.9 Hz, 1H), | |
| | 1.86-1.64 (m, 2H), 1.56-1.13 (m, 4H) | |
| 11-B | (400 MHz, d₆-DMSO) δ 10.97 (d, J = 2.6 Hz, | 1-B |
| | 1H), 8.21-8.12 (m, 1H), 7.68 (dd, J = 8.4, | |
| | 16.3 Hz, 1H), 7.29 (br d, J = 5.4 Hz, 1H), | |
| | 7.22-7.13 (m, 1H), 7.08-6.99 (m, 1H), | |
| | 6.96-6.88 (m, 1H), 5.15-4.89 (m, 2H), | |
| | 4.57-4.20 (m, 7H), 4.04-3.98 (m, 1H), | |
| | 3.86-3.83 (m, 3H), 2.97-2.84 (m, 1H), | |
| | 2.63-2.57 (m, 1H), 2.43-2.35 (m, 1H), | |
| | 2.28-2.03 (m, 2H), 1.98 (dt, J = 2.3, 5.1 Hz, | |
| | 1H), 1.89-1.63 (m, 2H), 1.51-1.18 (m, | |
| | 4H). | |
| 16-B | (400 MHz, d₄-MeOH) δ 1.38-1.62 (m, 4H), | 2-B |
| | 1.78-1.90 (m, 1H), 1.92-2.04 (m, 1H), 2.11- | |
| | 2.31 (m, 2H), 2.34-2.56 (m, 2H), 2.74-2.98 | |
| | (m, 2H), 3.66-3.76 (m, 1H), 3.91 (s, 3H), | |
| | 4.07-4.18 (m, 1H), 4.34-4.60 (m, 6H), 5.09- | |
| | 5.19 (m, 2H), 6.86 (d, J = 8.75 Hz, 1H), 7.14- | |
| | 7.32 (m, 2H), 7.74-7.87 (m, 2H), 8.12 (d, | |
| | J = 2.25 Hz, 1H). | |
| 21-B | (400 MHz, d₄-methanol) δ 1.47-1.65 (m, | 2-B |
| | 4H), 1.81-2.03 (m, 2H), 2.05-2.21 (m, 2H), | |
| | 2.24-2.40 (m, 1H), 2.41-2.55 (m, 1H), 2.74- | |
| | 2.83 (m, 1H), 2.84-2.97 (m, 1H), 3.78 (br dd, | |
| | J = 15.62, 2.70 Hz, 1H), 4.41-4.53 (m, 3H), | |
| | 4.55-4.72 (m, 3H), 4.73-4.82 (m, 1H), 5.09- | |
| | 5.18 (m, 2H), 7.22-7.28 (m, 1H), 7.31 (s, | |
| | 1H), 7.78 (d, J = 8.41 Hz, 1H), 7.83-7.94 (m, | |
| | 2H), 8.09-8.19 (m, 2H), 8.80-8.87 (m, 1H) | |
| 26-B | (400 MHz, d₆-DMSO) δ 10.98 (s, 1H), 10.57- | 1-B |
| | 10.35 (m, 1H), 9.13-8.87 (m, 2H), 7.67 (br | |
| | d, J = 8.5 Hz, 1H), 7.33-7.22 (m, 1H), 7.13 | |
| | (br d, J = 8.5 Hz, 1H), 5.08 (br dd, J = 4.4, | |
| | 12.9 Hz, 1H), 4.97-4.02 (m, 8H), 3.68- | |
| | 3.55 (m, 1H), 2.97-2.85 (m, 1H), 2.60 (br d, | |
| | J = 17.5 Hz, 1H), 2.45-2.35 (m, 1H), 2.27- | |
| | 2.04 (m, 2H), 2.03-1.93 (m, 1H), 1.87- | |
| | 1.65 (m, 2H), 1.54-1.17 (m, 4H). | |
| 31-B | (400 MHz, d₆-DMSO) δ 11.03-10.94 (m, | 2-B |
| | 1H), 8.71-8.57 (m, 2H), 7.73-7.64 (m, | |
| | 1H), 7.62-7.55 (m, 1H), 7.54-7.49 (m, | |
| | 1H), 7.33-7.26 (m, 1H), 7.22-7.13 (m, | |
| | 1H), 5.14-5.04 (m, 1H), 4.93 (s, 1H), 4.57- | |
| | 4.22 (m, 7H), 4.14-4.08 (m, 1H), 2.97- | |
| | 2.86 (m, 1H), 2.63-2.57 (m, 1H), 2.43- | |
| | 2.36 (m, 1H), 2.29-2.16 (m, 1H), 2.16- | |
| | 2.04 (m, 1H), 2.03-1.93 (m, 1H), 1.89- | |
| | 1.63 (m, 2H), 1.50-1.16 (m, 4H). | |
| 36-B | (400 MHz, d₆-DMSO) δ 1.26-1.52 (m, 4 H) | 2-B |
| | 1.60-1.73 (m, 1 H) 1.77-1.93 (m, 2 H) | |
| | 1.94-2.03 (m, 1 H) 2.04-2.15 (m, 1 H) | |
| | 2.35-2.46 (m, 1 H) 2.60 (br d, J = 17.12 Hz, | |
| | 1 H) 2.85-2.97 (m, 1 H) 3.80 (br s, 1 H) | |
| | 4.06-4.16 (m, 1 H) 4.26-4.39 (m, 3 H) | |
| | 4.41-4.50 (m, 3 H) 4.97 (br s, 1 H) 5.09 (m, | |
| | 1 H) 7.15-7.22 (m, 1 H) 7.29 (s, 1 H) 7.51- | |
| | 7.59 (m, 1 H) 7.63-7.72 (m, 1 H) 8.03 (br d, | |
| | J = 8.31 Hz, 1 H) 8.55-8.62 (m, 1 H) 8.64- | |
| | 8.73 (m, 1 H) 10.04-10.23 (m, 1 H) 10.98 | |
| | (s, 1 H) | |
| 46-B | (400 MHz, d₆-DMSO) δ 1.25-1.53 (m, 4 H) | 2-B |
| | 1.57-1.84 (m, 2 H) 1.86-2.25 (m, 3 H) | |
| | 2.35-2.45 (m, 1 H) 2.60 (br d, J = 17.12 Hz, | |
| | 2 H) 2.84-2.98 (m, 1 H) 4.18-4.41 (m, 4 | |
| | H) 4.42-4.66 (m, 3 H) 5.01-5.12 (m, 2 H) | |
| | 7.11-7.23 (m, 1 H) 7.25-7.34 (m, 1 H) | |
| | 7.45-7.52 (m, 1 H) 7.64-7.72 (m, 1 H) | |
| | 8.83-8.87 (m, 2 H) 9.91-10.15 (m, 1 H) | |
| | 10.98 (s, 1 H) | |
| 51-B | (400 MHz, do-DMSO) δ 10.98 (s, 1H), 10.14- | 2-B |
| | 9.94 (m, 1H), 8.71-8.58 (m, 1H), 7.82 (dt, | |
| | J = 1.7, 7.7 Hz, 1H), 7.73-7.65 (m, 1H), | |
| | 7.45-7.33 (m, 2H), 7.32-7.25 (m, 1H), | |
| | 7.23-7.14 (m, 1H), 5.14-4.98 (m, 2H), | |
| | 4.57-4.47 (m, 1H), 4.46-4.33 (m, 3H), | |
| | 4.32-4.24 (m, 2H), 4.18-4.13 (m, 1H), | |
| | 3.66-3.62 (m, 1H), 2.99-2.83 (m, 1H), | |
| | 2.59 (br dd, J = 1.1, 17.0 Hz, 1H), 2.42- | |
| | 2.36 (m, 1H), 2.12-1.88 (m, 3H), 1.86- | |
| | 1.74 (m, 1H), 1.73-1.57 (m, 1H), 1.55- | |
| | 1.30 (m, 4H). | |
| 56-B | (400 MHz, d₆-DMSO) δ 10.98 (s, 1H), 10.43 | 2-B |
| | (br d, J = 3.3 Hz, 1H), 8.67 (br t, J = 5.1 Hz, | |
| | 1H), 7.81-7.55 (m, 3H), 7.29 (br s, 1H), | |
| | 7.16 (br dd, J = 8.2, 17.8 Hz, 1H), 5.18- | |
| | 4.66 (m, 2H), 4.63-4.31 (m, 5H), 4.31- | |
| | 4.11 (m, 3H), 2.97-2.85 (m, 1H), 2.65- | |
| | 2.59 (m, 3H), 2.59-2.51 (m, 2H), 2.45- | |
| | 2.34 (m, 1H), 2.28-2.10 (m, 1H), 2.06- | |
| | 1.94 (m, 1H), 1.92-1.80 (m, 1H), 1.77- | |
| | 1.61 (m, 1H), 1.54-1.18 (m, 4H). | |
| 61-B | (400 MHz, d₆-DMSO) δ 1.30 (m, 1 H) 1.37- | 1-B |
| | 1.47 (m, 1 H) 1.51-1.67 (m, 5 H) 1.94- | |
| | 2.09 (m, 2 H) 2.36-2.43 (m, 1 H) 2.60 (br s, | |
| | 2 H) 2.83-2.96 (m, 1 H) 3.12-3.20 (m, 2 | |
| | H) 3.62-3.68 (m, 1 H) 3.72 (br s, 2 H) 4.21- | |
| | 4.28 (m, 1 H) 4.33-4.40 (m, 1 H) 4.64 (br | |
| | d, J = 6.68 Hz, 1 H) 5.06 (m, 1 H) 6.90-6.96 | |
| | (m, 1 H) 7.01-7.08 (m, 2 H) 7.12-7.16 (m, | |
| | 1 H) 7.18 (d, J = 1.91 Hz, 1 H) 7.31 (d, J = 8.11 | |
| | Hz, 1 H) 7.60 (m, 2 H) 10.81 (br s, 1 H) | |
| | 10.96 (s, 1 H) | |
| 66-B | (400 MHz, d₄-MeOH) δ 1.38-1.61 (m, 4H), | 1-B |
| | 1.81-2.00 (m, 2H), 2.03-2.21 (m, 2H), 2.24- | |
| | 2.40 (m, 1H), 2.40-2.57 (m, 1H), 2.73-2.83 | |
| | (m, 1H), 2.85-2.99 (m, 1H), 3.58-3.72 (m, | |
| | 1H), 4.38-4.55 (m, 2H), 4.56-4.80 (m, 3H), | |
| | 4.90-5.00 (m, 2H), 5.03-5.19 (m, 2H), 7.14- | |
| | 7.35 (m, 2H), 7.63-7.86 (m, 3H), 7.90-8.01 | |
| | (m, 2H), 8.16 (br d, J = 8.28 Hz, 1H), 8.98- | |
| | 9.06 (m, 1H) | |
| 71-B | (400 MHz, d₆-DMSO) δ 1.22-1.49 (m, 4 H) | 1-B |
| | 1.62-1.85 (m, 2 H) 1.88-2.09 (m, 2 H) | |
| | 2.12-2.26 (m, 1 H) 2.40 (m, 1 H) 2.60 (br d, | |
| | J = 18.60 Hz, 1 H) 2.92 (m, 1 H) 3.78 (br d, | |
| | J = 1.91 Hz, 1 H) 4.23-4.35 (m, 1 H) 4.36- | |
| | 4.47 (m, 2 H) 4.49-4.60 (m, 3 H) 4.60- | |
| | 4.69 (m, 1 H) 5.00 (br s, 1 H) 5.09 (m, 1 H) | |
| | 7.08 (d, J = 7.15 Hz, 1 H) 7.15-7.24 (m, 1 H) | |
| | 7.31 (br s, 1 H) 7.35-7.42 (m, 1 H) 7.50 (d, | |
| | J = 8.58 Hz, 1 H) 7.65-7.74 (m, 1 H) 8.16 (s, | |
| | 1 H) 10.05-10.61 (m, 1 H) 10.96-11.02 | |
| | (m, 1 H) 13.14-13.29 (m, 1 H). | |
| 76-B | (400 MHz, d₆-DMSO) δ 10.99 (s, 1H), 10.53- | 1-B |
| | 10.28 (m, 1H), 8.82-8.61 (m, 2H), 7.79- | |
| | 7.59 (m, 1H), 7.35-7.24 (m, 1H), 7.24- | |
| | 7.10 (m, 1H), 5.09 (dd, J = 5.0, 13.4 Hz, | |
| | 1H), 4.65-4.39 (m, 3H), 4.39-4.35 (m, | |
| | 1H), 4.35-4.20 (m, 3H), 4.09-3.96 (m, | |
| | 1H), 3.95-3.88 (m, 3H), 3.85-3.71 (m, | |
| | 1H), 3.03-2.81 (m, 1H), 2.56-2.53 (m, | |
| | 2H), 2.30-2.19 (m, 1H), 2.17-2.05 (m, | |
| | 1H), 2.04-1.93 (m, 1H), 1.87-1.70 (m, | |
| | 2H), 1.55-1.12 (m, 4H). | |
| 81-B | (400 MHz, d₆-DMSO) δ 10.97 (s, 1H), 10.81- | 3-B |
| | 10.27 (m, 1H), 9.14-8.71 (m, 1H), 8.49- | |
| | 8.34 (m, 1H), 7.90 (br d, J = 11.1 Hz, 1H), | |
| | 7.74-7.57 (m, 2H), 7.36-7.02 (m, 2H), | |
| | 6.54 (s, 1H), 6.34 (br d, J = 5.4 Hz, 1H), 5.79 | |
| | (s, 1H), 5.44-5.20 (m, 1H), 5.14-4.93 (m, | |
| | 1H), 4.84-4.19 (m, 5H), 3.78-3.60 (m, | |
| | 1H), 2.99-2.84 (m, 1H), 2.61 (br s, 2H), | |
| | 2.43-2.35 (m, 1H), 2.30-2.09 (m, 2H), | |
| | 2.04-1.91 (m, 1H), 1.85-1.66 (m, 2H), | |
| | 1.59-1.13 (m, 4H). | |
| 85-B | (400 MHz, d₆-DMSO) δ 10.96 (s, 1H), 8.46 | 4-B |
| | (d, J = 5.9 Hz, 2H), 7.60 (d, J = 8.4 Hz, 1H), | |
| | 7.31 (d, J = 5.9 Hz, 2H), 7.18 (s, 1H), 7.05 | |
| | (dd, J = 1.8, 8.4 Hz, 1H), 5.06 (dd, J = 4.9, | |
| | 13.3 Hz, 1H), 4.62 (br d, J = 7.8 Hz, 1H), | |
| | 4.42-4.32 (m, 1H), 4.29-4.19 (m, 1H), | |
| | 3.67-3.47 (m, 3H), 3.19-3.05 (m, 2H), | |
| | 2.97-2.83 (m, 1H), 2.63-2.56 (m, 2H), | |
| | 2.37 (br dd, J = 4.3, 13.6 Hz, 1H), 2.06- | |
| | 1.92 (m, 2H), 1.71-1.46 (m, 5H), 1.44- | |
| | 1.24 (m, 2H) | |
| 86-B | (400 MHz, d₆-DMSO) δ 10.97 (s, 1H), 8.46 | 4-B |
| | (d, J = 5.8 Hz, 2H), 8.16 (s, 1H), 7.61 (d, J = | |
| | 8.4 Hz, 1H), 7.38-7.25 (m, 2H), 7.18 (s, | |
| | 1H), 7.06 (dd, J = 1.9, 8.4 Hz, 1H), 5.07 (dd, | |
| | J = 5.1, 13.3 Hz, 1H), 4.64 (br d, J = 7.6 Hz, | |
| | 1H), 4.40-4.32 (m, 1H), 4.29-4.22 (m, | |
| | 1H), 3.65-3.53 (m, 3H), 3.24-3.11 (m, | |
| | 2H), 2.97-2.85 (m, 1H), 2.68-2.56 (m, | |
| | 2H), 2.43-2.33 (m, 1H), 2.10-1.92 (m, | |
| | 2H), 1.70-1.47 (m, 5H), 1.44-1.26 (m, 2H) | |
| 87-B | (400 MHz, d₆-DMSO) δ 10.97 (s, 1H), 8.87 | 3-B |
| | (br d, J = 3.0 Hz, 1H), 8.32 (br d, J = 8.3 Hz, | |
| | 1H), 8.00-7.82 (m, 2H), 7.62 (br dd, J = | |
| | 8.4, 12.1 Hz, 2H), 7.47 (dd, J = 4.1, 8.3 Hz, | |
| | 1H), 7.21 (br s, 1H), 7.14-7.01 (m, 1H), | |
| | 5.06 (dd, J = 5.1, 13.3 Hz, 1H), 4.45-4.19 | |
| | (m, 4H), 3.80-3.63 (m, 3H), 3.38 (br s, 1H), | |
| | 3.30-3.24 (m, 1H), 2.96-2.84 (m, 1H), | |
| | 2.58 (br d, J = 17.1 Hz, 1H), 2.44-2.35 (m, | |
| | 1H), 2.08-1.83 (m, 3H), 1.67 (br s, 2H), | |
| | 1.46-1.35 (m, 2H), 1.30-1.13 (m, 2H). | |
| 88-B | m/z (ESI⁺) 525.1 (M + H)⁺. | 2-B |
| 89-B | m/z (ESI⁺) 525.1 (M + H)⁺. | 2-B |
| 90-B | m/z (ESI⁺) 525.1 (M + H)⁺. | 2-B |
| 91-B | m/z (ESI⁺) 525.1 (M + H)⁺. | 2-B |
| 92-B | m/z (ESI⁺) 510.1 (M + H)⁺. | 2-B |
| 93-B | m/z (ESI⁺) 510.1 (M + H)⁺. | 2-B |
| 94-B | m/z (ESI⁺) 510.1 (M + H)⁺. | 2-B |
| 95-B | m/z (ESI⁺) 509.1 M⁺. | 2-B |
| 96-B | m/z (ESI⁺) 509.1 M⁺. | 2-B |
| 97-B | m/z (ESI⁺) 509.1 M⁺. | 2-B |
| 98-B | m/z (ESI⁺) 509.1 M⁺. | 2-B |
| 99-B | m/z (ESI⁺) 509.1 M⁺. | 2-B |
| 100-B | m/z (ESI⁺) 509.1 M⁺. | 2-B |
| 101-B | m/z (ESI⁺) 509.1 M⁺. | 2-B |
| 102-B | m/z (ESI⁺) 493.1 (M + H)⁺. | 2-B |
| 103-B | m/z (ESI⁺) 493.1 (M + H)⁺. | 2-B |
| 104-B | m/z (ESI⁺) 493.1 (M + H)⁺. | 2-B |
| 105-B | m/z (ESI⁺) 509.1, 511.1 M+. | 2-B |
| 106-B | (400 MHz, d₆-DMSO) δ 10.97 (s, 1H), 10.50- | 1-B |
| | 10.30 (m, 1H), 9.05 (d, J = 4.4 Hz, 2H), | |
| | 7.67 (br d, J = 8.3 Hz, 1H), 7.34-7.22 (m, | |
| | 1H), 7.13 (br d, J = 8.5 Hz, 1H), 5.08 (br dd, | |
| | J = 3.9, 12.9 Hz, 1H), 4.96-4.70 (m, 1H), | |
| | 4.53-4.05 (m, 6H), 3.73-3.53 (m, 1H), | |
| | 2.96-2.86 (m, 1H), 2.62 (br s, 1H), 2.41- | |
| | 2.36 (m, 1H), 2.22-2.08 (m, 2H), 2.03- | |
| | 1.93 (m, 1H), 1.82-1.64 (m, 2H), 1.54- | |
| | 1.15 (m, 5H). | |
| 107-B | (400 MHz, d₆-DMSO) δ 10.98 (s, 1H), 10.15- | 1-B |
| | 9.88 (m, 1H), 9.10-9.00 (m, 2H), 7.72- | |
| | 7.65 (m, 1H), 7.33-7.26 (m, 1H), 7.24- | |
| | 7.13 (m, 1H), 5.14-4.97 (m, 2H), 4.82- | |
| | 4.64 (m, 1H), 4.61-4.53 (m, 1H), 4.49- | |
| | 4.24 (m, 5H), 3.81-3.56 (m, 1H), 2.97- | |
| | 2.87 (m, 1H), 2.60 (br d, J = 18.0 Hz, 1H), | |
| | 2.39 (br dd, J = 4.4, 13.4 Hz, 1H), 2.14- | |
| | 1.87 (m, 3H), 1.83-1.59 (m, 2H), 1.51- | |
| | 1.25 (m, 4H). | |
| 108-B | m/z (ESI⁺) 554.1, 556.0 (M + H)⁺. | 1-B |
| 109-B | m/z (ESI⁺) 554.1, 556.0 (M + H)⁺. | 1-B |
| 110-B | m/z (ESI⁺) 554.1, 556.0 (M + H)⁺. | 1-B |
| 111-B | m/z (ESI⁺) 554.1, 556.0 (M + H)⁺. | 1-B |
| 112-B | (400 MHz, d₆-DMSO) δ 10.97 (s, 1H), 8.41- | 2-B |
| | 8.22 (m, 2H), 8.16 (br s, 1H), 7.61 (d, J = 8.4 | |
| | Hz, 1H), 7.29-7.17 (m, 2H), 7.10-7.03 (m, | |
| | 1H), 5.06 (dd, J = 5.1, 13.3 Hz, 1H), 4.65 (br | |
| | d, J = 7.3 Hz, 1H), 4.41-4.33 (m, 1H), 4.29- | |
| | 4.22 (m, 1H), 3.74-3.68 (m, 3H), 3.31- | |
| | 3.09 (m, 2H), 2.98-2.83 (m, 1H), 2.67- | |
| | 2.59 (m, 1H), 2.57 (br s, 1H), 2.46-2.32 (m, | |
| | 1H), 2.15-2.10 (m, 3H), 2.07-1.80 (m, | |
| | 2H), 1.72-1.48 (m, 4H), 1.44-1.15 (m, | |
| | 2H). | |
| 113-B | (400 MHz, d₆-DMSO) δ 11.57 (br s, 1H), | 2-B |
| | 10.96 (s, 1H), 8.20-8.10 (m, 1H), 7.60 (d, J = | |
| | 8.4 Hz, 1H), 7.38 (br s, 1H), 7.19 (s, 1H), | |
| | 7.09-7.02 (m, 1H), 7.01-6.92 (m, 1H), | |
| | 6.51-6.43 (m, 1H), 5.06 (dd, J = 5.0, 13.3 | |
| | Hz, 1H), 4.66 (br d, J = 7.4 Hz, 1H), 4.40- | |
| | 4.23 (m, 2H), 4.01-3.87 (m, 2H), 3.79 (br | |
| | dd, J = 6.1, 13.9 Hz, 2H), 3.33-3.28 (m, | |
| | 2H), 2.95-2.84 (m, 1H), 2.59 (br d, J = 17.5 | |
| | Hz, 2H), 2.44-2.31 (m, 1H), 2.11-1.83 (m, | |
| | 2H), 1.74-1.47 (m, 4H), 1.42-1.13 (m, | |
| | 2H). | |
| 114-B | m/z (ESI⁺) 510.1, 512.0 (M + H)⁺. | 1-B |
| 115-B | m/z (ESI⁺) 510.1, 512.1 (M + H)⁺. | 1-B |
| 116-B | m/z (ESI⁺) 510.1, 512.0 (M + H)⁺. | 1-B |
| 117-B | m/z (ESI⁺) 510.1, 512.0 (M + H)⁺. | 1-B |
| 118-B | (400 MHz, d₆-DMSO) δ 10.96 (s, 1H), 9.20 | 1-B |
| | (s, 2H), 7.60 (d, J = 8.4 Hz, 1H), 7.17 (s, | |
| | 1H), 7.02 (dd, J = 1.6, 8.4 Hz, 1H), 5.06 (dd, | |
| | J = 5.0, 13.3 Hz, 1H), 4.46-4.31 (m, 1H), | |
| | 4.29-4.20 (m, 2H), 3.94-3.81 (m, 1H), | |
| | 3.73-3.57 (m, 3H), 3.46 (br t, J = 6.8 Hz, | |
| | 1H), 2.99-2.83 (m, 1H), 2.64-2.55 (m, | |
| | 1H), 2.44-2.35 (m, 2H), 2.06-1.92 (m, | |
| | 2H), 1.89-1.79 (m, 1H), 1.65 (br s, 2H), | |
| | 1.42-1.20 (m, 3H), 1.17-1.03 (m, 1H). | |
| 119-B | (400 MHz, d₆-DMSO) δ 10.96 (s, 1H), 9.20 | 1-B |
| | (s, 2H), 7.60 (d, J = 8.4 Hz, 1H), 7.16 (s, | |
| | 1H), 7.02 (dd, J = 1.8, 8.6 Hz, 1H), 5.06 (dd, | |
| | J = 4.9, 13.3 Hz, 1H), 4.45-4.31 (m, 1H), | |
| | 4.32-4.15 (m, 2H), 3.92-3.82 (m, 1H), | |
| | 3.76-3.56 (m, 3H), 3.46 (t, J = 6.9 Hz, 1H), | |
| | 2.97-2.83 (m, 1H), 2.59 (br d, J = 17.6 Hz, | |
| | 1H), 2.45-2.39 (m, 2H), 2.05-1.92 (m, | |
| | 2H), 1.90-1.78 (m, 1H), 1.65 (br s, 2H), | |
| | 1.34-1.20 (m, 3H), 1.15-1.06 (m, 1H). | |
| 120-B | (400 MHz, d₆-DMSO) δ 10.96 (s, 1H), 9.20 | 1-B |
| | (s, 2H), 7.60 (d, J = 8.4 Hz, 1H), 7.16 (s, | |
| | 1H), 7.02 (dd, J = 1.9, 8.5 Hz, 1H), 5.06 (dd, | |
| | J = 5.1, 13.2 Hz, 1H), 4.46-4.33 (m, 1H), | |
| | 4.28-4.18 (m, 2H), 3.95-3.81 (m, 1H), | |
| | 3.75-3.55 (m, 3H), 3.46 (t, J = 7.0 Hz, 1H), | |
| | 2.98-2.82 (m, 1H), 2.59 (br d, J = 16.9 Hz, | |
| | 1H), 2.46-2.38 (m, 2H), 2.05-1.93 (m, | |
| | 2H), 1.88-1.79 (m, 1H), 1.65 (br d, J = 4.4 | |
| | Hz, 2H), 1.43-1.21 (m, 3H), 1.16-1.02 (m, | |
| | 1H). | |
| 121-B | (400 MHz, d₆-DMSO) δ 11.02 (s, 1H), 9.27 | 1-B |
| | (s, 2H), 7.67 (d, J = 8.4 Hz, 1H), 7.26-7.19 | |
| | (m, 1H), 7.11-7.00 (m, 1H), 5.12 (dd, J = | |
| | 5.0, 13.3 Hz, 1H), 4.50-4.39 (m, 1H), 4.36- | |
| | 4.26 (m, 2H), 3.99-3.89 (m, 1H), 3.82- | |
| | 3.64 (m, 3H), 3.57-3.49 (m, 1H), 3.04- | |
| | 2.90 (m, 1H), 2.65 (br d, J = 18.6 Hz, 1H), | |
| | 2.49-2.42 (m, 2H), 2.09-1.87 (m, 3H), | |
| | 1.79-1.64 (m, 2H), 1.46-1.30 (m, 3H), | |
| | 1.20-1.09 (m, 1H). | |
| 122-B | (400 MHz, d₆-DMSO) δ 10.96 (s, 1H), 9.20 | 1-B |
| | (s, 2H), 7.60 (d, J = 8.4 Hz, 1H), 7.16 (s, | |
| | 1H), 7.02 (dd, J = 1.9, 8.5 Hz, 1H), 5.06 (dd, | |
| | J = 4.9, 13.3 Hz, 1H), 4.44-4.32 (m, 1H), | |
| | 4.30-4.16 (m, 2H), 3.97-3.83 (m, 1H), | |
| | 3.72-3.56 (m, 3H), 3.52-3.42 (m, 1H), | |
| | 2.98-2.83 (m, 1H), 2.63-2.55 (m, 1H), | |
| | 2.44-2.36 (m, 2H), 2.05-1.92 (m, 2H), | |
| | 1.91-1.78 (m, 1H), 1.71-1.58 (m, 2H), | |
| | 1.43-1.23 (m, 3H), 1.16-1.04 (m, 1H). | |
| 123-B | (400 MHz, d₆-DMSO) δ 10.96 (s, 1H), 9.20 | 1-B |
| | (s, 2H), 7.60 (d, J = 8.4 Hz, 1H), 7.17 (s, | |
| | 1H), 7.02 (dd, J = 1.6, 8.4 Hz, 1H), 5.06 (dd, | |
| | J = 5.0, 13.3 Hz, 1H), 4.46-4.31 (m, 1H), | |
| | 4.29-4.20 (m, 2H), 3.94-3.81 (m, 1H), | |
| | 3.73-3.57 (m, 3H), 3.46 (br t, J = 6.8 Hz, | |
| | 1H), 2.99-2.83 (m, 1H), 2.64-2.55 (m, | |
| | 1H), 2.44-2.35 (m, 2H), 2.06-1.92 (m, | |
| | 2H), 1.89-1.79 (m, 1H), 1.65 (br s, 2H), | |
| | 1.42-1.20 (m, 3H), 1.17-1.03 (m, 1H). | |
| 124-B | m/z (ESI⁺) 543.1 (M + H)⁺. | 1-B |
| 125-B | m/z (ESI⁺) 543.1 (M + H)⁺. | 1-B |
| 126-B | m/z (ESI⁺) 543.1 (M + H)⁺. | 1-B |
| 127-B | m/z (ESI⁺) 543.1 (M + H)⁺. | 1-B |
| 128-B | (400 MHz, d₆-DMSO) δ 10.96 (s, 1H), 8.91 | 1-B |
| | (s, 1H), 8.12 (br d, J = 7.5 Hz, 1H), 7.59 (t, | |
| | J = 8.8 Hz, 2H), 7.17 (s, 1H), 7.03 (br d, J = | |
| | 7.5 Hz, 1H), 5.06 (dd, J = 5.0, 13.0 Hz, 1H), | |
| | 4.43-4.17 (m, 3H), 3.83-3.73 (m, 1H), | |
| | 3.70-3.58 (m, 2H), 3.53-3.43 (m, 1H), | |
| | 2.97-2.83 (m, 1H), 2.64-2.56 (m, 2H), | |
| | 2.37 (br dd, J = 4.0, 13.0 Hz, 2H), 2.03- | |
| | 1.93 (m, 2H), 1.89-1.80 (m, 1H), 1.66 (br s, | |
| | 2H), 1.43-1.25 (m, 3H), 1.18-1.04 (m, | |
| | 1H). | |
| 129-B | (400 MHz, d₆-DMSO) δ 10.96 (s, 1H), 8.90 | 1-B |
| | (s, 1H), 8.12 (dd, J = 2.0, 8.5 Hz, 1H), 7.59 | |
| | (t, J = 9.0 Hz, 2H), 7.17 (s, 1H), 7.03 (dd, | |
| | J = 2.0, 8.5 Hz, 1H), 5.06 (dd, J = 5.0, 13.5 | |
| | Hz, 1H), 4.45-4.15 (m, 3H), 3.83-3.73 (m, | |
| | 1H), 3.71-3.57 (m, 2H), 3.53-3.43 (m, | |
| | 1H), 2.96-2.83 (m, 1H), 2.59 (br d, J = 18.5 | |
| | Hz, 2H), 2.43-2.33 (m, 2H), 2.06-1.92 (m, | |
| | 2H), 1.89-1.79 (m, 1H), 1.65 (br s, 2H), | |
| | 1.45-1.20 (m, 3H), 1.12 (quin, J = 10.8 Hz, | |
| | 1H). | |
| 130-B | (400 MHz, d₆-DMSO) δ 11.02 (s, 1H), 8.97 | 1-B |
| | (s, 1H), 8.19 (dd, J = 1.8, 8.3 Hz, 1H), 7.72- | |
| | 7.60 (m, 2H), 7.24 (s, 1H), 7.10 (dd, J = 2.0, | |
| | 8.5 Hz, 1H), 5.13 (dd, J = 5.3, 13.3 Hz, 1H), | |
| | 4.51-4.24 (m, 3H), 3.84 (br d, J = 7.0 Hz, | |
| | 1H), 3.77-3.63 (m, 2H), 3.60-3.50 (m, | |
| | 1H), 3.03-2.90 (m, 1H), 2.70-2.62 (m, | |
| | 2H), 2.47-2.40 (m, 1H), 2.10-2.00 (m, | |
| | 2H), 1.91 (br d, J = 9.5 Hz, 1H), 1.72 (br s, | |
| | 2H), 1.50-1.27 (m, 4H), 1.24-1.09 (m, | |
| | 1H). | |
| 131-B | (400 MHz, d₆-DMSO) δ 10.96 (s, 1H), 8.90 | 1-B |
| | (s, 1H), 8.11 (dd, J = 2.3, 8.3 Hz, 1H), 7.59 | |
| | (t, J = 9.5 Hz, 2H), 7.16 (s, 1H), 7.03 (dd, J = | |
| | 1.8, 8.3 Hz, 1H), 5.05 (dd, J = 5.0, 13.0 | |
| | Hz, 1H), 4.42-4.18 (m, 3H), 3.82-3.72 (m, | |
| | 1H), 3.69-3.57 (m, 2H), 3.48 (br t, J = 7.0 | |
| | Hz, 1H), 2.96-2.83 (m, 1H), 2.70-2.59 (m, | |
| | 1H), 2.41-2.29 (m, 2H), 2.06-1.92 (m, | |
| | 2H), 1.89-1.78 (m, 1H), 1.65 (br s, 2H), | |
| | 1.43-1.20 (m, 4H), 1.17-1.00 (m, 1H). | |
| 132-B | (400 MHz, d₆-DMSO) δ 0.77-0.83 (m, 2 H) | 4-B |
| | 0.98-1.05 (m, 2 H) 1.06-1.14 (m, 1 H) | |
| | 1.20-1.32 (m, 2 H) 1.32-1.41 (m, 2 H) | |
| | 1.65 (br s, 2 H) 1.79-1.87 (m, 1 H) 1.87- | |
| | 1.94 (m, 1 H) 1.94-2.03 (m, 2 H) 2.39 (m, 2 | |
| | H) 2.59 (br d, J = 18.01 Hz, 1 H) 2.85-2.95 | |
| | (m, 1 H) 3.45-3.78 (m, 4 H) 4.22-4.31 (m, | |
| | 2 H) 4.32-4.41 (m, 1 H) 5.06 (m, 1 H) 7.02 | |
| | (br d, J = 8.00 Hz, 1 H) 7.16 (br s, 1 H) 7.60 | |
| | (d, J = 8.38 Hz, 1 H) 8.50 (s, 2 H) 10.96 (s, 1 | |
| | H). | |
| 133-B | (400 MHz, d₆-DMSO) δ 0.77-0.83 (m, 2 H) | 4-B |
| | 0.97-1.05 (m, 2 H) 1.05-1.15 (m, 1 H) | |
| | 1.19-1.31 (m, 2 H) 1.32-1.43 (m, 2 H) | |
| | 1.65 (br s, 2 H) 1.78-1.87 (m, 1 H) 1.87- | |
| | 1.93 (m, 1 H) 1.94-2.04 (m, 2 H) 2.38 (m, 2 | |
| | H) 2.59 (br d, J = 16.76 Hz, 1 H) 2.84-2.96 | |
| | (m, 1 H) 3.48-3.77 (m, 4 H) 4.21-4.30 (m, | |
| | 2 H) 4.34-4.42 (m, 1 H) 5.06 (m, 1 H) 7.02 | |
| | (br d, J = 8.00 Hz, 1 H) 7.16 (s, 1 H) 7.60 (d, | |
| | J = 8.38 Hz, 1 H) 8.49 (s, 2 H) 10.96 (s, 1 H). | |
| 134-B | (400 MHz, d₆-DMSO) δ 0.78-0.83 (m, 2 H) | 4-B |
| | 0.98-1.05 (m, 2 H) 1.07-1.15 (m, 1 H) | |
| | 1.20-1.32 (m, 2 H) 1.33-1.42 (m, 2 H) | |
| | 1.65 (br s, 2 H) 1.80-1.87 (m, 1 H) 1.88- | |
| | 1.94 (m, 1 H) 1.95-2.04 (m, 2 H) 2.40 (m, 2 | |
| | H) 2.59 (br d, J = 17.89 Hz, 1 H) 2.86-2.95 | |
| | (m, 1 H) 3.51-3.75 (m, 4 H) 4.21-4.30 (m, | |
| | 2 H) 4.33-4.42 (m, 1 H) 5.07 (m, 1 H) 7.02 | |
| | (br d, J = 8.25 Hz, 1 H) 7.17 (s, 1 H) 7.61 (d, | |
| | J = 8.38 Hz, 1 H) 8.50 (s, 2 H) 10.96 (s, 1 H). | |
| 135-B | (400 MHz, d₆-DMSO) δ 0.78-0.82 (m, 2 H) | 4-B |
| | 0.97-1.04 (m, 2 H) 1.19-1.31 (m, 2 H) | |
| | 1.32-1.40 (m, 2 H) 1.65 (br s, 2 H) 1.80- | |
| | 1.87 (m, 1 H) 1.87-1.93 (m, 1 H) 1.94- | |
| | 2.04 (m, 2 H) 2.38 (m, 2 H) 2.59 (br d, | |
| | J = 16.88 Hz, 1 H) 2.84-2.95 (m, 1 H) 3.24- | |
| | 3.27 (m, 2 H) 3.53-3.65 (m, 2 H) 3.66-3.75 | |
| | (m, 1 H) 4.19-4.30 (m, 2 H) 4.33-4.42 (m, | |
| | 1 H) 5.06 (m, 1 H) 7.02 (m, 1 H) 7.16 (s, 1 | |
| | H) 7.60 (d, J = 8.38 Hz, 1 H) 8.49 (s, 2 H) | |
| | 10.96 (s, 1 H). | |
| 136-B | (400 MHz, d₆-DMSO) δ 10.98 (s, 1H), 8.58- | 1-B |
| | 8.41 (m, 2H), 7.62 (d, J = 8.4 Hz, 1H), 7.38- | |
| | 7.30 (m, 2H), 7.23-7.15 (m, 1H), 7.09- | |
| | 7.02 (m, 1H), 5.08 (br dd, J = 4.9, 13.3 Hz, | |
| | 1H), 4.70-4.57 (m, 1H), 4.44-4.36 (m, | |
| | 1H), 4.30-4.24 (m, 1H), 3.67-3.48 (m, | |
| | 3H), 3.21-3.04 (m, 2H), 2.97-2.87 (m, | |
| | 1H), 2.63 (br s, 2H), 2.41 (br d, J = 4.8 Hz, | |
| | 1H), 2.08-1.94 (m, 2H), 1.70-1.39 (m, | |
| | 5H), 1.35-1.21 (m, 2H). | |
| 137-B | (400 MHz, d₆-DMSO) δ 10.97 (s, 1H), 10.50- | 1-B |
| | 10.30 (m, 1H), 9.05 (d, J = 4.4 Hz, 2H), | |
| | 7.67 (br d, J = 8.3 Hz, 1H), 7.34-7.22 (m, | |
| | 1H), 7.13 (br d, J = 8.5 Hz, 1H), 5.08 (br dd, | |
| | J = 3.9, 12.9 Hz, 1H), 4.96-4.70 (m, 1H), | |
| | 4.53-4.05 (m, 6H), 3.73-3.53 (m, 1H), | |
| | 2.96-2.86 (m, 1H), 2.62 (br s, 1H), 2.41- | |
| | 2.36 (m, 1H), 2.22-2.08 (m, 2H), 2.03- | |
| | 1.93 (m, 1H), 1.82-1.64 (m, 2H), 1.54- | |
| | 1.15 (m, 5H) | |
| 138-B | (400 MHz, d₆-DMSO) δ 10.98 (s, 1H), 10.15- | 1-B |
| | 9.88 (m, 1H), 9.10-9.00 (m, 2H), 7.72- | |
| | 7.65 (m, 1H), 7.33-7.26 (m, 1H), 7.24- | |
| | 7.13 (m, 1H), 5.14-4.97 (m, 2H), 4.82- | |
| | 4.64 (m, 1H), 4.61-4.53 (m, 1H), 4.49- | |
| | 4.24 (m, 5H), 3.81-3.56 (m, 1H), 2.97- | |
| | 2.87 (m, 1H), 2.60 (br d, J = 18.0 Hz, 1H), | |
| | 2.39 (br dd, J = 4.4, 13.4 Hz, 1H), 2.14- | |
| | 1.87 (m, 3H), 1.83-1.59 (m, 2H), 1.51- | |
| | 1.25 (m, 4H) | |
| 139-B | m/z (ESI⁺) 510.1, 512.1 (M + H)⁺. | 1-B |
| 140-B | m/z (ESI⁺) 510.1, 512.1 (M + H)⁺. | 1-B |
| 141-B | (400 MHz, d₆-DMSO) δ 1.61-1.72 (m, 1 H) | 1-B |
| | 1.73-1.91 (m, 3 H) 1.94-2.03 (m, 1 H) | |
| | 2.07-2.20 (m, 2 H) 2.39 (m, 1 H) 2.62 (br s, | |
| | 1 H) 2.84-2.97 (m, 1 H) 4.12-4.34 (m, 3 | |
| | H) 4.36-4.67 (m, 5 H) 5.08 (m, 2 H) 7.16 | |
| | (br d, J = 8.50 Hz, 1 H) 7.25 (s, 1 H) 7.65- | |
| | 7.73 (m, 1 H) 7.95-8.02 (m, 1 H) 8.17- | |
| | 8.26 (m, 1 H) 8.78-8.85 (m, 1 H) 10.15- | |
| | 10.43 (m, 1 H) 10.98 (s, 1 H). | |
| 142-B | m/z (ESI⁺) 476.1 (M + H)⁺. | 1-B |
| 143-B | m/z (ESI⁺) 476.1 (M + H)⁺. | 1-B |
| 144-B | m/z (ESI⁺) 477.1 (M + H)⁺. | 1-B |
| 145-B | m/z (ESI⁺) 477.1 (M + H)⁺. | 1-B |
| 146-B | m/z (ESI⁺) 525.1 (M + H)⁺. | 1-B |
| 147-B | (400 MHz, d₆-DMSO) δ 1.61-1.72 (m, 1 H) | 1-B |
| | 1.74-1.92 (m, 3 H) 1.94-2.03 (m, 1 H) | |
| | 2.05-2.21 (m, 2 H) 2.35-2.45 (m, 1 H) | |
| | 2.62 (br s, 1 H) 2.84-2.98 (m, 1 H) 4.03- | |
| | 4.16 (m, 1 H) 4.23-4.40 (m, 3 H) 4.43- | |
| | 4.62 (m, 4 H) 5.03-5.15 (m, 2 H) 7.10- | |
| | 7.18 (m, 1 H) 7.24 (s, 1 H) 7.61-7.72 (m, 2 | |
| | H) 8.24-8.30 (m, 1 H) 9.01-9.08 (m, 1 H) | |
| | 10.20-10.40 (m, 1 H) 10.97 (s, 1 H) | |
| 148-B | m/z (ESI⁺) 473.1 (M + H)⁺. | 1-B |
| 149-B | m/z (ESI⁺) 491.1 (M + H)⁺. | 1-B |
| 150-B | m/z (ESI⁺) 491.1 (M + H)⁺. | 1-B |
| 151-B | m/z (ESI⁺) 465.1 (M + H)⁺. | 1-B |
| 152-B | m/z (ESI⁺) 525.1 (M + H)⁺. | 1-B |
| 153-B | m/z (ESI⁺) 493.1 (M + H)⁺. | 1-B |
| 154-B | m/z (ESI⁺) 529.1 (M + H)⁺. | 1-B |
| 155-B | m/z (ESI⁺) 509.1, 511.1 (M + H)⁺. | 1-B |
| 156-B | (400 MHz, d₆-DMSO) δ 1.53-1.72 (m, 2 H) | 2-B |
| | 1.73-1.84 (m, 2 H) 2.09-2.31 (m, 2 H) | |
| | 2.82 (s, 4 H) 3.02-3.10 (m, 1 H) 3.94-4.22 | |
| | (m, 1 H) 4.27-4.42 (m, 4 H) 4.44-4.65 (m, | |
| | 3 H) 4.91 (br s, 1 H) 7.08 (br d, J = 9.01 Hz, 1 | |
| | H) 7.15-7.22 (m, 1 H) 7.62 (d, J = 8.50 Hz, 1 | |
| | H) 8.95-9.04 (m, 2 H) 10.43-10.72 (m, 1 | |
| | H) 10.93 (d, J = 2.00 Hz, 1 H). | |
| 157-B | (400 MHz, d₆-DMSO) δ 1.61-1.70 (m, 1 H) | 2-B |
| | 1.74-1.86 (m, 3 H) 2.07-2.19 (m, 2 H) | |
| | 2.82 (s, 4 H) 3.04-3.09 (m, 1 H) 3.99-4.11 | |
| | (m, 1 H) 4.34 (s, 3 H) 4.43-4.51 (m, 2 H) | |
| | 4.53-4.67 (m, 2 H) 5.09 (br d, J = 1.50 Hz, 1 | |
| | H) 7.09-7.17 (m, 1 H) 7.21 (br d, J = 5.50 | |
| | Hz, 1 H) 7.64 (m, 1 H) 9.00 (s, 1 H) 9.03 (s, | |
| | 1 H) 10.14-10.33 (m, 1 H) 10.94 (d, J = 2.00 | |
| | Hz, 1 H). | |
| 158-B | m/z (ESI⁺) 480.3 (M + H)⁺. | 1-B |
| 159-B | (400 MHz, d₆-DMSO) δ 1.09-1.16 (m, 1 H) | 1-B |
| | 1.21-1.30 (m, 1 H) 1.38 (br t, J = 8.76 Hz, 2 | |
| | H) 1.65 (br d, J = 4.50 Hz, 2 H) 1.79-1.88 | |
| | (m, 1 H) 1.95-2.03 (m, 1 H) 2.81 (s, 4 H) | |
| | 3.06 (br s, 1 H) 3.15 (br t, J = 6.50 Hz, 2 H) | |
| | 3.51 (m, 2 H) 3.57-3.62 (m, 1 H) 3.66 (br t, | |
| | J = 6.75 Hz, 1 H) 4.30 (s, 3 H) 7.04 (m, 1 H) | |
| | 7.16 (s, 1 H) 7.33 (d, J = 6.00 Hz, 2 H) 7.55 | |
| | (d, J = 8.50 Hz, 1 H) 8.46 (d, J = 6.00 Hz, 2 H) | |
| | 10.92 (br s, 1 H). | |
| 160-B | (400 MHz, d₆-DMSO) δ 1.30 (br s, 1 H) 1.36- | 1-B |
| | 1.44 (m, 1 H) 1.45-1.53 (m, 1 H) 1.54- | |
| | 1.70 (m, 4 H) 2.02 (m, 1 H) 2.58-2.64 (m, 1 | |
| | H) 2.81 (s, 4 H) 3.03-3.11 (m, 2 H) 3.13- | |
| | 3.18 (m, 1 H) 3.48-3.57 (m, 2 H) 3.58- | |
| | 3.64 (m, 1 H) 4.29 (s, 2 H) 4.62 (br d, J = 7.50 | |
| | Hz, 1 H) 7.05 (m, 1 H) 7.16 (d, J = 1.50 Hz, 1 | |
| | H) 7.29-7.33 (m, 2 H) 7.55 (d, J = 8.50 Hz, 1 | |
| | H) 8.42-8.48 (m, 2 H) 10.92 (br s, 1 H). | |
| 161-B | m/z (ESI⁺) 465.1 (M + H)⁺. | 1-B |
| 162-B | m/z (ESI⁺) 509.1, 511.0 (M + H)⁺. | 1-B |
| 163-B | m/z (ESI⁺) 509.1, 511.1 (M + H)⁺. | 1-B |
| 164-B | m/z (ESI⁺) 509.1, 511.1 (M + H)⁺. | 1-B |
| 165-B | m/z (ESI⁺) 509.1, 511.1 (M + H)⁺. | 1-B |
| 166-B | m/z (ESI⁺) 509.1, 511.1 (M + H)⁺. | 1-B |
| 167-B | m/z (ESI⁺) 493.1 (M + H)⁺. | 1-B |
| 168-B | m/z (ESI⁺) 509.1, 511.1 (M + H)⁺. | 1-B |
| 169-B | (400 MHz, d₆-DMSO) δ 10.97 (s, 1H), 10.60- | 1-B |
| | 10.18 (m, 1H), 9.00-8.87 (m, 1H), 8.39 (br | |
| | d, J = 7.9 Hz, 1H), 8.19-7.98 (m, 2H), 7.72- | |
| | 7.63 (m, 2H), 7.55 (dd, J = 4.2, 8.2 Hz, | |
| | 1H), 7.31-7.05 (m, 2H), 5.14-5.00 (m, | |
| | 1H), 4.97-4.86 (m, 1H), 4.68-4.46 (m, | |
| | 3H), 4.44-4.14 (m, 5H), 2.98-2.83 (m, | |
| | 1H), 2.62 (br s, 2H), 2.42-2.35 (m, 1H), | |
| | 2.25-2.13 (m, 1H), 2.06-1.94 (m, 1H), | |
| | 1.90-1.54 (m, 4H). | |
| 170-B | (400 MHz, d₆-DMSO) δ 10.97 (s, 1H), 10.58- | 1-B |
| | 10.17 (m, 1H), 8.21-8.12 (m, 1H), 7.74- | |
| | 7.57 (m, 1H), 7.21 (s, 1H), 7.10 (br d, J = 8.3 | |
| | Hz, 1H), 7.02 (d, J = 5.4 Hz, 1H), 6.96-6.89 | |
| | (m, 1H), 5.08 (dd, J = 5.0, 13.2 Hz, 1H), | |
| | 4.87 (br d, J = 3.5 Hz, 1H), 4.59-4.21 (m, | |
| | 6H), 4.19-4.00 (m, 2H), 3.85 (s, 3H), 2.98- | |
| | 2.83 (m, 1H), 2.60 (br d, J = 18.6 Hz, 1H), | |
| | 2.39 (br dd, J = 4.3, 13.6 Hz, 1H), 2.31- | |
| | 2.22 (m, 1H), 2.20-2.09 (m, 1H), 2.03- | |
| | 1.94 (m, 1H), 1.83-1.73 (m, 2H), 1.72- | |
| | 1.63 (m, 1H), 1.62-1.52 (m, 1H). | |
| 171-B | (400 MHz, d₆-DMSO) δ 1.24-1.48 (m, 4 H) | 1-B |
| | 1.66-1.84 (m, 2 H) 2.09-2.25 (m, 2 H) | |
| | 2.82 (br d, J = 2.00 Hz, 4 H) 3.07 (br s, 1 H) | |
| | 3.57-3.76 (m, 1 H) 4.03-4.40 (m, 5 H) | |
| | 4.42-4.58 (m, 2 H) 4.59-4.98 (m, 1 H) | |
| | 7.13 (br d, J = 9.01 Hz, 1 H) 7.24 (br d, | |
| | J = 4.00 Hz, 1 H) 7.62 (m, 1 H) 8.99 (d, | |
| | J = 4.50 Hz, 2 H) 10.33-10.57 (m, 1 H) | |
| | 10.93 (s, 1 H) | |
| 172-B | (400 MHz, d₆-DMSO) δ 1.28-1.53 (m, 4 H) | 1-B |
| | 1.58-1.72 (m, 1 H) 1.74-1.85 (m, 1 H) | |
| | 1.86-1.97 (m, 1 H) 1.99-2.10 (m, 1 H) | |
| | 2.83 (br s, 4 H) 3.07 (br s, 1 H) 3.58-3.78 | |
| | (m, 1 H) 4.18-4.36 (m, 4 H) 4.46 (m, 2 H) | |
| | 4.52-4.80 (m, 1 H) 4.98-5.09 (m, 1 H) | |
| | 7.14-7.23 (m, 1 H) 7.25-7.30 (m, 1 H) | |
| | 7.60-7.67 (m, 1 H) 8.98-9.01 (m, 2 H) | |
| | 9.93-10.18 (m, 1 H) 10.94 (d, J = 2.00 Hz, 1 | |
| | H). | |
| 173-B | m/z (ESI⁺) 509.1, 511.1 (M + H)⁺. | 1-B |
| 174-B | m/z (ESI⁺) 479.1 (M + H)⁺. | 1-B |
| 175-B | m/z (ESI⁺) 479.1 (M + H)⁺. | 1-B |
| 176-B | (400 MHz, d₆-DMSO) δ 11.00-10.94 (m, | 2-B |
| | 1H), 9.20-8.82 (m, 1H), 8.06-7.84 (m, | |
| | 1H), 7.73-7.51 (m, 2H), 7.33-7.19 (m, | |
| | 1H), 7.17-7.05 (m, 1H), 5.37-4.97 (m, | |
| | 2H), 4.69-4.14 (m, 5H), 3.01-2.72 (m, | |
| | 2H), 2.59 (br d, J = 19.4 Hz, 2H), 2.42-2.36 | |
| | (m, 1H), 2.27-1.93 (m, 3H), 1.85-1.68 (m, | |
| | 2H), 1.52-1.12 (m, 5H). | |
| 177-B | m/z (ESI⁺) 516.2 (M + H)⁺. | 1-B |
| 178-B | (400 MHz, d₆-DMSO) δ 11.00 (s, 1H), 10.13 | 1-B |
| | (br d, J = 3.1 Hz, 1H), 8.94 (br d, J = 1.8 Hz, | |
| | 2H), 8.44-8.31 (m, 1H), 7.79-7.61 (m, | |
| | 1H), 7.31 (s, 1H), 7.21 (dd, J = 1.8, 8.4 Hz, | |
| | 1H), 5.10 (dd, J = 4.8, 13.4 Hz, 1H), 4.97 (br | |
| | s, 1H), 4.62-4.49 (m, 2H), 4.48-4.36 (m, | |
| | 2H), 4.35-4.16 (m, 3H), 3.82 (br t, J = 9.8 | |
| | Hz, 1H), 3.02-2.85 (m, 1H), 2.53 (d, J = 1.9 | |
| | Hz, 2H), 2.16-1.95 (m, 2H), 1.94-1.78 (m, | |
| | 2H), 1.67 (br d, J = 11.8 Hz, 1H), 1.54-1.24 | |
| | (m, 5H). | |
| 179-B | m/z (ESI⁺) 500.1 (M + H)⁺. | 1-B |
| 180-B | m/z (ESI⁺) 500.1 (M + H)⁺. | 1-B |
| 181-B | (400 MHz, d₆-DMSO) δ 1.57-1.73 (m, 1 H) | 1-B |
| | 1.76-1.92 (m, 2 H) 1.94-2.03 (m, 1 H) | |
| | 2.06-2.25 (m, 1 H) 2.36-2.45 (m, 1 H) | |
| | 2.58-2.64 (m, 2 H) 2.85-2.98 (m, 1 H) | |
| | 4.01-4.16 (m, 1 H) 4.21-4.46 (m, 5 H) | |
| | 4.48-4.67 (m, 2 H) 5.08 (m, 1 H) 6.98- | |
| | 7.06 (m, 1 H) 7.07-7.22 (m, 2 H) 7.24- | |
| | 7.28 (m, 1 H) 7.36-7.42 (m, 1 H) 7.50- | |
| | 7.53 (m, 1 H) 7.58 (d, J = 8.11 Hz, 1 H) 7.66- | |
| | 7.73 (m, 1 H) 10.03-10.27 (m, 1 H) 10.98 | |
| | (s, 1 H) 11.11-11.19 (m, 1 H). | |
| 182-B | m/z (ESI⁺) 543.1 (M + H)⁺. | 1-B |
| 183-B | (400 MHz, d₆-DMSO) δ 10.99 (s, 1H), 10.61- | 1-B |
| | 10.45 (m, 1H), 8.84 (s, 1H), 8.28 (br d, J = | |
| | 8.1 Hz, 1H), 8.04-7.94 (m, 1H), 7.68 (dd, J = | |
| | 1.9, 8.1 Hz, 1H), 7.35-7.28 (m, 1H), 7.23- | |
| | 7.12 (m, 1H), 5.09 (dd, J = 4.8, 13.4 Hz, | |
| | 1H), 4.66-4.48 (m, 3H), 4.44-4.15 (m, | |
| | 5H), 3.87-3.71 (m, 1H), 2.99-2.85 (m, | |
| | 1H), 2.63 (br s, 1H), 2.44-2.37 (m, 1H), | |
| | 2.30-2.07 (m, 2H), 2.05-1.93 (m, 1H), | |
| | 1.88-1.70 (m, 2H), 1.54-1.40 (m, 1H), | |
| | 1.39-1.19 (m, 3H). | |
| 184-B | (400 MHz, d₆-DMSO) δ 10.97 (s, 1H), 10.59- | 1-B |
| | 10.29 (m, 1H), 8.69 (s, 2H), 7.66 (d, J = 8.4 | |
| | Hz, 1H), 7.27 (br d, J = 4.6 Hz, 1H), 7.12 | |
| | (dd, J = 2.0, 8.3 Hz, 1H), 5.15-5.00 (m, | |
| | 1H), 4.81 (s, 1H), 4.67-4.13 (m, 7H), 3.75- | |
| | 3.57 (m, 1H), 2.97-2.84 (m, 1H), 2.64- | |
| | 2.55 (m, 1H), 2.42-2.35 (m, 1H), 2.31- | |
| | 2.25 (m, 3H), 2.24-2.08 (m, 2H), 2.02- | |
| | 1.91 (m, 1H), 1.84-1.66 (m, 2H), 1.49- | |
| | 1.13 (m, 4H). | |
| 185-B | (400 MHz, d₆-DMSO) δ 10.98 (br s, 1H), | 1-B |
| | 10.57-10.24 (m, 1H), 8.69 (d, J = 5.1 Hz, | |
| | 1H), 7.67 (d, J = 8.5 Hz, 1H), 7.39-7.24 (m, | |
| | 2H), 7.17-7.10 (m, 1H), 5.08 (br dd, J = | |
| | 4.4, 13.2 Hz, 1H), 4.95-4.70 (m, 1H), 4.67- | |
| | 4.17 (m, 6H), 4.03 (br dd, J = 4.6, 8.0 Hz, | |
| | 1H), 3.71-3.55 (m, 1H), 3.00-2.84 (m, | |
| | 1H), 2.64-2.56 (m, 2H), 2.47 (br s, 3H), | |
| | 2.43-2.35 (m, 1H), 2.27-2.08 (m, 2H), | |
| | 2.03-1.92 (m, 1H), 1.85-1.66 (m, 2H), | |
| | 1.55-1.12 (m, 4H). | |
| 186-B | (400 MHz, d₆-DMSO) δ 10.96 (s, 1H), 9.20 | 1-B |
| | (s, 2H), 7.60 (d, J = 8.4 Hz, 1H), 7.16 (s, | |
| | 1H), 7.02 (dd, J = 1.9, 8.5 Hz, 1H), 5.06 (dd, | |
| | J = 4.9, 13.3 Hz, 1H), 4.44-4.32 (m, 1H), | |
| | 4.30-4.16 (m, 2H), 3.97-3.83 (m, 1H), | |
| | 3.72-3.56 (m, 3H), 3.52-3.42 (m, 1H), | |
| | 2.98-2.83 (m, 1H), 2.63-2.55 (m, 1H), | |
| | 2.44-2.36 (m, 2H), 2.05-1.92 (m, 2H), | |
| | 1.91-1.78 (m, 1H), 1.71-1.58 (m, 2H), | |
| | 1.43-1.23 (m, 3H), 1.16-1.04 (m, 1H) | |
| 187-B | (400 MHz, d₆-DMSO) δ 10.96 (s, 1H), 9.20 | 1-B |
| | (s, 2H), 7.60 (d, J = 8.4 Hz, 1H), 7.17 (s, | |
| | 1H), 7.02 (dd, J = 1.6, 8.4 Hz, 1H), 5.06 (dd, | |
| | J = 5.0, 13.3 Hz, 1H), 4.46-4.31 (m, 1H), | |
| | 4.29-4.20 (m, 2H), 3.94-3.81 (m, 1H), | |
| | 3.73-3.57 (m, 3H), 3.46 (br t, J = 6.8 Hz, | |
| | 1H), 2.99-2.83 (m, 1H), 2.64-2.55 (m, | |
| | 1H), 2.44-2.35 (m, 2H), 2.06-1.92 (m, | |
| | 2H), 1.89-1.79 (m, 1H), 1.65 (br s, 2H), | |
| | 1.42-1.20 (m, 3H), 1.17-1.03 (m, 1H) | |
| 188-B | (400 MHz, d₆-DMSO) δ 10.99 (s, 1H), 10.21- | 1-B |
| | 9.83 (m, 1H), 8.74-8.60 (m, 1H), 7.74- | |
| | 7.58 (m, 1H), 7.45-7.01 (m, 3H), 5.16- | |
| | 4.98 (m, 2H), 4.79-4.13 (m, 7H), 3.79- | |
| | 3.58 (m, 1H), 2.98-2.85 (m, 1H), 2.60 (br d, | |
| | J = 19.0 Hz, 2H), 2.48 (br s, 3H), 2.39 (br | |
| | dd, J = 4.2, 13.0 Hz, 1H), 2.12-1.85 (m, | |
| | 3H), 1.83-1.58 (m, 2H), 1.53-1.23 (m, | |
| | 4H). | |
| 189-B | (400 MHz, d₆-DMSO) δ 10.97 (s, 1H), 10.24- | 1-B |
| | 9.91 (m, 1H), 8.69 (s, 2H), 7.62 (br d, J = | |
| | 8.2 Hz, 1H), 7.33-7.27 (m, 1H), 7.23-7.13 | |
| | (m, 1H), 5.14-4.99 (m, 2H), 4.64-4.13 (m, | |
| | 7H), 3.70-3.56 (m, 1H), 2.98-2.83 (m, | |
| | 1H), 2.65-2.55 (m, 1H), 2.43-2.35 (m, | |
| | 1H), 2.31-2.26 (m, 3H), 2.15-1.86 (m, | |
| | 3H), 1.84-1.57 (m, 2H), 1.54-1.23 (m, | |
| | 4H). | |
| 190-B | (400 MHz, d₆-DMSO) δ 11.05 (s, 1H), 10.62 | 1-B |
| | (br s, 1H), 8.83-8.71 (m, 1H), 7.80-7.70 | |
| | (m, 1H), 7.51-7.31 (m, 2H), 7.30-7.17 (m, | |
| | 1H), 5.23-5.03 (m, 1H), 4.68-4.53 (m, | |
| | 2H), 4.49 (br t, J = 8.0 Hz, 2H), 4.44 (br d, | |
| | J = 8.1 Hz, 1H), 4.40-4.34 (m, 1H), 4.32 (br | |
| | d, J = 6.4 Hz, 1H), 4.27-4.14 (m, 1H), 3.83- | |
| | 3.66 (m, 1H), 3.05-2.91 (m, 1H), 2.72 (d, | |
| | J = 3.8 Hz, 3H), 2.59 (br d, J = 1.9 Hz, 2H), | |
| | 2.34-2.17 (m, 1H), 2.16-1.94 (m, 2H), | |
| | 1.93-1.65 (m, 2H), 1.62-1.23 (m, 4H). | |
| 191-B | (400 MHz, d₆-DMSO) δ 10.95 (br s, 1H), | 1-B |
| | 8.71 (d, J = 1.4 Hz, 1H), 8.12-8.07 (m, 1H), | |
| | 7.84 (d, J = 8.0 Hz, 1H), 7.60 (d, J = 8.5 Hz, | |
| | 1H), 7.18 (s, 1H), 7.04 (dd, J = 2.0, 8.4 Hz, | |
| | 1H), 5.05 (dd, J = 5.1, 13.3 Hz, 1H), 4.40- | |
| | 4.33 (m, 1H), 4.30-4.22 (m, 2H), 3.72- | |
| | 3.67 (m, 1H), 3.66-3.61 (m, 2H), 3.23- | |
| | 3.16 (m, 3H), 2.95-2.85 (m, 1H), 2.60 (br d, | |
| | J = 2.8 Hz, 1H), 2.39-2.34 (m, 1H), 2.03- | |
| | 1.93 (m, 2H), 1.87-1.81 (m, 1H), 1.66 (br s, | |
| | 2H), 1.39 (br t, J = 8.1 Hz, 2H), 1.27-1.21 | |
| | (m, 1H), 1.18-1.10 (m, 1H). | |
| 192-B | (400 MHz, d₆-DMSO) δ 10.99 (s, 1H), 10.14- | 1-B |
| | 9.91 (m, 1H), 9.07-8.98 (m, 1H), 8.32- | |
| | 8.23 (m, 1H), 7.72-7.68 (m, 1H), 7.64 (d, J = | |
| | 8.1 Hz, 1H), 7.32-7.27 (m, 1H), 7.20 (br | |
| | d, J = 9.1 Hz, 1H), 5.12-5.05 (m, 1H), 5.03 | |
| | (br d, J = 1.0 Hz, 1H), 4.61-4.52 (m, 1H), | |
| | 4.50-4.41 (m, 2H), 4.39 (br d, J = 7.2 Hz, | |
| | 1H), 4.35-4.24 (m, 3H), 3.71-3.60 (m, | |
| | 1H), 2.98-2.86 (m, 1H), 2.62 (br s, 1H), | |
| | 2.39 (br dd, J = 4.3, 12.9 Hz, 1H), 2.13- | |
| | 1.87 (m, 3H), 1.87-1.75 (m, 1H), 1.73- | |
| | 1.59 (m, 1H), 1.54-1.28 (m, 4H). | |
| 193-B | (400 MHz, d₆-DMSO) δ 10.97 (s, 1H), 8.87 | 3-B |
| | (br d, J = 3.0 Hz, 1H), 8.32 (br d, J = 8.3 Hz, | |
| | 1H), 8.00-7.82 (m, 2H), 7.62 (br dd, J = | |
| | 8.4, 12.1 Hz, 2H), 7.47 (dd, J = 4.1, 8.3 Hz, | |
| | 1H), 7.21 (br s, 1H), 7.14-7.01 (m, 1H), | |
| | 5.06 (dd, J = 5.1, 13.3 Hz, 1H), 4.45-4.19 | |
| | (m, 4H), 3.80-3.63 (m, 3H), 3.38 (br s, 1H), | |
| | 3.30-3.24 (m, 1H), 2.96-2.84 (m, 1H), | |
| | 2.58 (br d, J = 17.1 Hz, 1H), 2.44-2.35 (m, | |
| | 1H), 2.08-1.83 (m, 3H), 1.67 (br s, 2H), | |
| | 1.46-1.35 (m, 2H), 1.30-1.13 (m, 2H). | |
| 194-B | m/z (ESI⁺) 511.0 (M + H)⁺. | 1-B |
| 195-B | m/z (ESI⁺) 511.0 (M + H)⁺. | 1-B |
| 196-B | m/z (ESI⁺) 476.1 (M + H)⁺. | 1-B |
| 197-B | m/z (ESI⁺) 476.1 (M + H)⁺. | 1-B |
| 198-B | (400 MHz, d₆-DMSO) δ 10.99 (s, 1H), 8.91- | 1-B |
| | 8.81 (m, 1H), 8.75 (s, 1H), 7.71 (dd, J = 8.5, | |
| | 11.9 Hz, 1H), 7.39-7.28 (m, 1H), 7.21 (br t, | |
| | J = 9.5 Hz, 1H), 5.19-5.01 (m, 1H), 4.96 (br | |
| | s, 1H), 4.54 (br d, J = 3.0 Hz, 2H), 4.49- | |
| | 4.37 (m, 3H), 4.36-4.25 (m, 2H), 3.01- | |
| | 2.86 (m, 2H), 2.34 (d, J = 4.4 Hz, 3H), 2.11- | |
| | 2.07 (m, 2H), 2.04-1.95 (m, 2H), 1.94- | |
| | 1.78 (m, 2H), 1.78-1.63 (m, 1H), 1.53- | |
| | 1.18 (m, 5H). | |
| 199-B | (400 MHz, d₆-DMSO) δ 11.83 (br s, 1H), | 1-B |
| | 11.00 (br d, J = 2.1 Hz, 1H), 8.36-8.24 (m, | |
| | 1H), 7.70 (dd, J = 8.4, 14.5 Hz, 1H), 7.52 (br | |
| | s, 1H), 7.32 (br s, 1H), 7.27-7.07 (m, 2H), | |
| | 6.50 (br s, 1H), 5.19-4.95 (m, 2H), 4.54 (br | |
| | d, J = 6.2 Hz, 4H), 4.45-4.35 (m, 2H), 4.34- | |
| | 4.22 (m, 2H), 3.05-2.85 (m, 1H), 2.41 (br | |
| | dd, J = 4.5, 13.1 Hz, 1H), 2.29-2.05 (m, | |
| | 2H), 2.04-1.88 (m, 2H), 1.85-1.63 (m, | |
| | 2H), 1.55-1.17 (m, 4H). | |
| 200-B | m/z (ESI⁺) 476.1, 498.1 (M + H)⁺. | 1-B |
| 201-B | m/z (ESI⁺) 491.1 (M + H)⁺. | 1-B |
| 202-B | m/z (ESI⁺) 491.1 (M + H)⁺. | 1-B |
| 203-B | m/z (ESI⁺) 512.1 (M + H)⁺. | 1-B |
| 204-B | (400 MHz, d₆-DMSO) δ 1.25-1.39 (m, 4H), | 3-B |
| | 1.43-1.50 (m, 1H), 1.72-1.77 (m, 1H), 1.79- | |
| | 1.84 (m, 1H), 1.94-2.03 (m, 2H), 2.15 (br d, | |
| | J = 2.38 Hz, 1H), 2.24 (br s, 1H), 2.85-3.00 | |
| | (m, 1H), 3.71-3.87 (m, 1H), 4.04-4.16 (m, | |
| | 1H), 4.24-4.31 (m, 1H), 4.33-4.55 (m, 6H), | |
| | 5.03-5.14 (m, 1H), 7.14-7.23 (m, 1H), 7.27- | |
| | 7.33 (m, 1H), 7.43-7.49 (m, 1H), 7.52 (s, | |
| | 1H), 7.64-7.71 (m, 1H), 7.83-7.89 (m, 1H), | |
| | 8.06 (s, 1H), 10.93-11.00 (m, 1H), 13.05- | |
| | 13.15 (m, 1H). | |
| 205-B | m/z (ESI⁺) 461.1 (M + H)⁺. | 1-B |
| 206-B | m/z (ESI⁺) 461.1 (M + H)⁺. | 1-B |
| 207-B | m/z (ESI⁺) 461.1 (M + H)⁺. | 1-B |
| 208-B | m/z (ESI⁺) 461.1 (M + H)⁺. | 1-B |
| 209-B | m/z (ESI⁺) 461.1 (M + H)⁺. | 1-B |
| 210-B | m/z (ESI⁺) 461.1 (M + H)⁺. | 1-B |
| 211-B | (400 MHz, d₆-DMSO) δ 1.22-1.49 (m, 4 H) | 1-B |
| | 1.62-1.85 (m, 2 H) 1.88-2.09 (m, 2 H) | |
| | 2.12-2.26 (m, 1 H) 2.40 (m, 1 H) 2.60 (br d, | |
| | J = 18.60 Hz, 1 H) 2.92 (m, 1 H) 3.78 (br d, | |
| | J = 1.91 Hz, 1 H) 4.23-4.35 (m, 1 H) 4.36- | |
| | 4.47 (m, 2 H) 4.49-4.60 (m, 3 H) 4.60- | |
| | 4.69 (m, 1 H) 5.00 (br s, 1 H) 5.09 (m, 1 H) | |
| | 7.08 (d, J = 7.15 Hz, 1 H) 7.15-7.24 (m, 1 H) | |
| | 7.31 (br s, 1 H) 7.35-7.42 (m, 1 H) 7.50 (d, | |
| | J = 8.58 Hz, 1 H) 7.65-7.74 (m, 1 H) 8.16 (s, | |
| | 1 H) 10.05-10.61 (m, 1 H) 10.96-11.02 | |
| | (m, 1 H) 13.14-13.29 (m, 1 H). | |
| 212-B | (400 MHz, d₄-methanol) | 2-B |
| | δ 1.40-1.57 (m, 4H), 1.77-1.89 (m, 1H), | |
| | 1.91-2.08 (m, 2H), 2.12-2.20 (m, 1H), 2.22- | |
| | 2.32 (m, 1H), 2.41-2.56 (m, 1H), 2.74-2.83 | |
| | (m, 1H), 2.86-2.97 (m, 1H), 3.72 (br d, | |
| | J = 11.01 Hz, 1H), 4.21-4.33 (m, 1H), 4.38- | |
| | 4.50 (m, 3H), 4.51-4.63 (m, 3H), 5.00-5.06 | |
| | (m, 1H), 5.09-5.17 (m, 1H), 7.14-7.26 (m, | |
| | 1H), 7.29 (s, 1H), 7.74-7.81 (m, 1H), 8.86- | |
| | 8.90 (m, 2H), 9.14 (s, 1H). | |

### BIOLOGICAL EXAMPLES

### Cereblon (CRBN) Target Engagement

HEK293T cells were harvested ca. 75% confluent with trypsin and plated (500,000 cells/well) in a 6-well tissue culture plate in 2 mL of Dulbecco's Modified Eagle Medium (DMEM)⁺10% Fetal Bovine Serum (FBS) and incubated overnight at 37° C.

The NanoLuc-CRBN fusion vector (Nluc-CRBN; Promega) contains the coding region of human E3 ligase component cereblon (CRBN) fused to the C-terminus of the NanoLuc luciferase coding region. A mixture of 10 ng Nluc-CRBN and 990 ng DDB1 Expression Vector (Promega) was added to 125 μL Opti-Minimum Essential Medium (Opti-MEM™; Thermo Fisher) along with 2 L P3000 reagent (Thermo Fisher) in a 1.5 mL epppendorf tube. This solution was added to Lipofectamine 3000 transfection reagent (5 μL; Thermo Fisher) in Opti-MEM (125 μL), mixed well, and incubated for 15 minutes at room temperature. The transfection mixture was added dropwise to cells and incubated overnight at 37° C., 5% CO₂. Following transfection, cells were washed once with PBS, and trypsin (250 μL) was added and incubated 30-45 see to dislodge cells. Complete media (2 mL) was added to resuspend cells to form a single cell suspension. Cells were centrifuged at 320×g for 5 min at room temperature, the supernatant was removed, and the cell pellet resuspended in Opti-MEM (3 mL; wash step repeated ×2). After final resuspension in 5 mL Opti-MEM, cells were counted and resuspended at 200,000 cells/mL in Opti-MEM.

Cereblon target engagement was monitored by Bioluminescence Resonance Energy Transfer (BRET) in transfected HEK-293T cells using the NanoBRET TE Intracellular E3 Ligase Assay (Promega). Briefly, 384-well plates (white opaque plates, Corning 3574, low binding surface) were seeded with transfected HEK-293T cells (38 μL/well). 2 μL of 10 μM CRBN tracer (diluted 1:5 in Tracer Dilution Buffer) was added to each well. Plates were centrifuged at 320×g for 1 min at room temperature. Test compounds were added in a 11-point dilution series (typically 10 μM to 100 pM) using a TECAN D300e Digital Dispenser. Plates were shaken for 2 minutes on a microplate shaker to mix compounds. Plates were centrifuged at 320×g for 1 min at room temperature, and subsequently incubated for 2 hours at 37° C.

After incubation, plates were allowed to cool to room temperature for 15 minutes. 20 μL of 3× Complete NanoBRET™ Nano-Glo® Substrate plus Inhibitor Solution (Promega, 1:166 Substrate and 1:500 dilution of Extracellular NanoLuc® Inhibitor diluted in Opti-MEM) were added to each well. Plates were incubated with shaking at room temperature for 3 minutes covered with foil. Plates were read on a CLARIOstar microplate reader (BMG LabTech), measuring at 450 nm (donor emission) and 610 nm (acceptor emission). The IC₅₀ values were determined by regression to best fit four-parameter logistic curves using GraphPad Prism.

### IKZF2 Degradation Assay

### Generation of Stable Cell Lines

Polycistronic plasmids were constructed for the mammalian expression of fluorescent reporter fusions of human transcription factors IKZF1 (Ikaros), IKZF2 (Helios), and IKZF3 (Aiolos). The respective protein sequences had their C-terminal end joined to a GGGGS linker repeated three times followed by mNeonGreen, P2A sequence, and mScarlet. The DNA sequences of the open reading frames are as follows:

### IKZF1-mNeonGreen-P2A-mScarlet Coding Sequence:

ATGGATGCTGATGAGGGTCAAGACATGTCCCAAGTTTCAGGGAAGGAAAG
CCCCCCTGTAAGCGATACTCCAGATGAGGGCGATGAGCCCATGCCGATCC
CCGAGGACCTCTCCACCACCTCGGGAGGACAGCAAAGCTCCAAGAGTGAC
AGAGTCGTGGCCAGTAATGTTAAAGTAGAGACTCAGAGTGATGAAGAGAA
TGGGCGTGCCTGTGAAATGAATGGGGAAGAATGTGCGGAGGATTTACGAA
TGCTTGATGCCTCGGGAGAGAAAATGAATGGCTCCCACAGGGACCAAGGC
AGCTCGGCTTTGTCGGGAGTTGGAGGCATTCGACTTCCTAACGGAAAACT
AAAGTGTGATATCTGTGGGATCATTTGCATCGGGCCCAATGTGCTCATGG
TTCACAAAAGAAGCCACACTGGAGAACGGCCCTTCCAGTGCAATCAGTGC
GGGGCCTCATTCACCCAGAAGGGCAACCTGCTCCGGCACATCAAGCTGCA
TTCCGGGGAGAAGCCCTTCAAATGCCACCTCTGCAACTACGCCTGCCGCC
GGAGGGACGCCCTCACTGGCCACCTGAGGACGCACTCCGTTGGTAAACCT
CACAAATGTGGATATTGTGGCCGAAGCTATAAACAGCGAAGCTCTTTAGA
GGAACATAAAGAGCGCTGCCACAACTACTTGGAAAGCATGGGCCTTCCGG
GCACACTGTACCCAGTCATTAAAGAAGAAACTAATCACAGTGAAATGGCA
GAAGACCTGTGCAAGATAGGATCAGAGAGATCTCTCGTGCTGGACAGACT
AGCAAGTAACGTCGCCAAACGTAAGAGCTCTATGCCTCAGAAATTTCTTG
GGGACAAGGGCCTGTCCGACACGCCCTACGACAGCAGCGCCAGCTACGAG
AAGGAGAACGAAATGATGAAGTCCCACGTGATGGACCAAGCCATCAACAA
CGCCATCAACTACCTGGGGGCCGAGTCCCTGCGCCCGCTGGTGCAGACGC
CCCCGGGCGGTTCCGAGGTGGTCCCGGTCATCAGCCCGATGTACCAGCTG
CACAAGCCGCTCGCGGAGGGCACCCCGCGCTCCAACCACTCGGCCCAGGA
CAGCGCCGTGGAGAACCTGCTGCTGCTCTCCAAGGCCAAGTTGGTGCCCT
CGGAGCGCGAGGCGTCCCCGAGCAACAGCTGCCAAGACTCCACGGACACC
GAGAGCAACAACGAGGAGCAGCGCAGCGGTCTCATCTACCTGACCAACCA
CATCGCCCCGCACGCGCGCAACGGGCTGTCGCTCAAGGAGGAGCACCGCG
CCTACGACCTGCTGCGCGCCGCCTCCGAGAACTCGCAGGACGCGCTCCGC
GTGGTCAGCACCAGCGGGGAGCAGATGAAGGTGTACAAGTGCGAACACTG
CCGGGTGCTCTTCCTGGATCACGTCATGTACACCATCCACATGGGCTGCC
ACGGCTTCCGTGATCCTTTTGAGTGCAACATGTGCGGCTACCACAGCCAG
GACCGGTACGAGTTCTCGTCGCACATAACGCGAGGGGAGCACCGCTTCCA
CATGAGCGGTGGAGGCGGTTCAGGTGGCGGAGGAAGCGGGGGAGGTGGAA
GTATGGTGTCTAAAGGAGAAGAAGATAATATGGCTTCTCTGCCTGCTACA
CACGAACTGCATATTTTCGGATCTATTAATGGCGTGGATTTCGATATGGT
GGGACAGGGAACAGGAAACCCTAACGATGGATACGAAGAACTGAATCTGA
AGTCTACCAAAGGAGATCTGCAATTCTCTCCTTGGATCCTGGTCCCTCAC
ATTGGATATGGATTCCATCAATATCTGCCTTACCCTGACGGAATGTCTCC
TTTCCAAGCTGCTATGGTCGATGGCTCTGGATATCAGGTCCACAGAACAA
TGCAATTTGAAGATGGCGCCTCTCTGACAGTGAATTACAGATATACATAC
GAAGGCTCTCATATTAAAGGCGAAGCCCAAGTGAAAGGCACAGGCTTTCC
TGCCGACGGCCCTGTGATGACCAACTCTCTGACCGCTGCCGATTGGTGCA
GATCTAAGAAGACCTACCCTAATGATAAAACCATTATCTCTACCTTCAAA
TGGTCTTACACAACAGGCAATGGCAAGAGATACAGATCTACCGCCAGAAC
CACCTATACCTTTGCTAAGCCTATGGCTGCCAATTACCTGAAAAATCAGC
CTATGTATGTGTTCAGAAAAACCGAACTGAAACACTCTAAAACCGAACTG
AATTTCAAAGAATGGCAAAAGGCTTTCACAGATGTGATGGGAATGGATGA
ACTGTACAAGGGAAGCGGAGCTACAAATTTTAGTCTTCTTAAACAAGCCG
GTGACGTGGAGGAAAATCCCGGCCCAATGGTGAGCAAGGGCGAGGCCGTG
ATCAAGGAGTTCATGCGCTTCAAGGTGCACATGGAGGGCAGCATGAACGG
CCACGAGTTCGAGATCGAGGGCGAGGGCGAGGGCCGCCCCTACGAGGGCA
CCCAGACCGCCAAGCTGAAGGTGACCAAGGGCGGCCCCCTGCCCTTCTCT
TGGGACATCCTGAGCCCCCAGTTCATGTACGGCAGCCGCGCCTTCACCAA
GCACCCCGCCGACATCCCCGACTACTACAAGCAGAGCTTCCCCGAGGGCT
TCAAGTGGGAGCGCGTGATGAACTTCGAGGACGGCGGCGCCGTGACCGTG
ACCCAGGACACCAGCCTGGAGGACGGCACCCTGATCTACAAGGTGAAGCT
GCGCGGCACCAACTTCCCCCCCGACGGCCCCGTGATGCAGAAGAAGACCA
TGGGCTGGGAGGCCAGCACCGAGCGCCTGTACCCCGAGGACGGCGTGCTG
AAGGGCGACATCAAGATGGCCCTGCGCCTGAAGGACGGCGGCAGATACCT
GGCCGACTTCAAGACCACCTACAAGGCCAAGAAGCCCGTGCAGATGCCCG
GCGCCTACAACGTGGACCGCAAGCTGGACATCACCAGCCACAACGAGGAC
TACACCGTGGTGGAGCAGTACGAGCGCAGCGAGGGCCGCCACAGCACCGG
CGGCATGGACGAGCTGTACAAGTGA

### IKZF2-mNeonGreen-P2A-mScarlet Coding Sequence:

ATGGAAACAGAGGCTATTGATGGCTATATAACGTGTGACAATGAGCTTTC
ACCCGAAAGGGAGCACTCCAATATGGCAATTGACCTCACCTCAAGCACAC
CCAATGGACAGCATGCCTCACCAAGTCACATGACAAGCACAAATTCAGTA
AAGCTAGAAATGCAGAGTGATGAAGAGTGTGACAGGAAACCCCTGAGCCG
TGAAGATGAGATCAGGGGCCATGATGAGGGTAGCAGCCTAGAAGAACCCC
TAATTGAGAGCAGCGAGGTGGCTGACAACAGGAAAGTCCAGGAGCTTCAA
GGCGAGGGAGGAATCCGGCTTCCGAATGGTAAACTGAAATGTGACGTCTG
TGGCATGGTTTGCATTGGGCCCAATGTGCTTATGGTACATAAAAGGAGTC
ACACTGGTGAACGCCCCTTCCACTGTAACCAGTGTGGAGCTTCTTTTACT
CAGAAGGGCAACCTTCTGAGACACATAAAGTTACACTCTGGAGAGAAGCC
GTTCAAATGTCCTTTCTGTAGCTACGCCTGTAGAAGAAGGGACGCCCTCA
CAGGACACCTCAGGACCCATTCTGTGGGTAAACCTCACAAGTGCAACTAC
TGTGGACGAAGCTACAAGCAGCGCAGTTCACTGGAGGAGCACAAGGAACG
CTGCCACAACTATCTCCAGAATGTCAGCATGGAGGCTGCTGGGCAGGTCA
TGAGTCACCATGTACCTCCTATGGAAGATTGTAAGGAACAAGAGCCTATT
ATGGACAACAATATTTCTCTGGTGCCTTTTGAGAGACCTGCTGTCATAGA
GAAGCTCACGGGGAATATGGGAAAACGTAAAAGCTCCACTCCACAAAAGT
TTGTGGGGGAAAAGCTCATGCGATTCAGCTACCCAGATATTCACTTTGAT
ATGAACTTAACATATGAGAAGGAGGCTGAGCTGATGCAGTCTCATATGAT
GGACCAAGCCATCAACAATGCAATCACCTACCTTGGAGCTGAGGCCCTTC
ACCCTCTGATGCAGCACCCGCCAAGCACAATCGCTGAAGTGGCCCCAGTT
ATAAGCTCAGCTTATTCTCAGGTCTATCATCCAAATAGGATAGAAAGACC
CATTAGCAGGGAAACTGCTGATAGTCATGAAAACAACATGGATGGCCCCA
TCTCTCTCATCAGACCAAAGAGTCGACCCCAGGAAAGAGAGGCCTCTCCC
AGCAATAGCTGCCTGGATTCCACTGACTCAGAAAGCAGCCATGATGACCA
CCAGTCCTACCAAGGACACCCTGCCTTAAATCCCAAGAGGAAACAAAGCC
CAGCTTACATGAAGGAGGATGTCAAAGCTTTGGATACTACCAAGGCTCCT
AAGGGCTCTCTGAAGGACATCTACAAGGTCTTCAATGGAGAAGGAGAACA
GATTAGGGCCTTCAAGTGTGAGCACTGCCGAGTCCTTTTCCTAGACCATG
TCATGTACACCATTCACATGGGTTGCCATGGCTACCGGGACCCACTGGAA
TGCAACATCTGTGGCTACAGAAGCCAGGACCGTTATGAGTTTTCATCACA
CATTGTTCGAGGGGAGCACACATTCCACCTCGACGGTGGAGGCGGTTCAG
GTGGCGGAGGAAGCGGGGGAGGTGGAAGTATGGTGTCTAAAGGAGAAGAA
GATAATATGGCTTCTCTGCCTGCTACACACGAACTGCATATTTTCGGATC
TATTAATGGCGTGGATTTCGATATGGTGGGACAGGGAACAGGAAACCCTA
ACGATGGATACGAAGAACTGAATCTGAAGTCTACCAAAGGAGATCTGCAA
TTCTCTCCTTGGATCCTGGTCCCTCACATTGGATATGGATTCCATCAATA
TCTGCCTTACCCTGACGGAATGTCTCCTTTCCAAGCTGCTATGGTCGATG
GCTCTGGATATCAGGTCCACAGAACAATGCAATTTGAAGATGGCGCCTCT
CTGACAGTGAATTACAGATATACATACGAAGGCTCTCATATTAAAGGCGA
AGCCCAAGTGAAAGGCACAGGCTTTCCTGCCGACGGCCCTGTGATGACCA
ACTCTCTGACCGCTGCCGATTGGTGCAGATCTAAGAAGACCTACCCTAAT
GATAAAACCATTATCTCTACCTTCAAATGGTCTTACACAACAGGCAATGG
CAAGAGATACAGATCTACCGCCAGAACCACCTATACCTTTGCTAAGCCTA
TGGCTGCCAATTACCTGAAAAATCAGCCTATGTATGTGTTCAGAAAAACC
GAACTGAAACACTCTAAAACCGAACTGAATTTCAAAGAATGGCAAAAGGC
TTTCACAGATGTGATGGGAATGGATGAACTGTACAAGGGAAGCGGAGCTA
CAAATTTTAGTCTTCTTAAACAAGCCGGTGACGTGGAGGAAAATCCCGGC
CCAATGGTGAGCAAGGGCGAGGCCGTGATCAAGGAGTTCATGCGCTTCAA
GGTGCACATGGAGGGCAGCATGAACGGCCACGAGTTCGAGATCGAGGGCG
AGGGCGAGGGCCGCCCCTACGAGGGCACCCAGACCGCCAAGCTGAAGGTG
ACCAAGGGCGGCCCCCTGCCCTTCTCTTGGGACATCCTGAGCCCCCAGTT
CATGTACGGCAGCCGCGCCTTCACCAAGCACCCCGCCGACATCCCCGACT
ACTACAAGCAGAGCTTCCCCGAGGGCTTCAAGTGGGAGCGCGTGATGAAC
TTCGAGGACGGCGGCGCCGTGACCGTGACCCAGGACACCAGCCTGGAGGA
CGGCACCCTGATCTACAAGGTGAAGCTGCGCGGCACCAACTTCCCCCCCG
ACGGCCCCGTGATGCAGAAGAAGACCATGGGCTGGGAGGCCAGCACCGAG
CGCCTGTACCCCGAGGACGGCGTGCTGAAGGGCGACATCAAGATGGCCCT
GCGCCTGAAGGACGGCGGCAGATACCTGGCCGACTTCAAGACCACCTACA
AGGCCAAGAAGCCCGTGCAGATGCCCGGCGCCTACAACGTGGACCGCAAG
CTGGACATCACCAGCCACAACGAGGACTACACCGTGGTGGAGCAGTACGA
GCGCAGCGAGGGCCGCCACAGCACCGGCGGCATGGACGAGCTGTACAAGT
GA

### IKZF3-mNeonGreen-P2A-mScarlet Coding Sequence:

ATGGAAGATATACAAACAAATGCGGAACTGAAAAGCACTCAGGAGCAGTC
TGTGCCCGCAGAAAGTGCAGCGGTTTTGAATGACTACAGTTTAACCAAAT
CTCATGAAATGGAAAATGTGGACAGTGGAGAAGGCCCAGCCAATGAAGAT
GAAGACATAGGAGATGATTCAATGAAAGTGAAAGATGAATACAGTGAAAG
AGATGAGAATGTTTTAAAGTCAGAACCCATGGGAAATGCAGAAGAGCCTG
AAATCCCTTACAGCTATTCAAGAGAATATAATGAATATGAAAACATTAAG
TTGGAGAGACATGTTGTCTCATTCGATAGTAGCAGGCCAACCAGTGGAAA
GATGAACTGCGATGTGTGTGGATTATCCTGCATCAGCTTCAATGTCTTAA
TGGTTCATAAGCGAAGCCATACTGGTGAACGCCCATTCCAGTGTAATCAG
TGTGGGGCATCTTTTACTCAGAAAGGTAACCTCCTCCGCCACATTAAACT
GCACACAGGGGAAAAACCTTTTAAGTGTCACCTCTGCAACTATGCATGCC
AAAGAAGAGATGCGCTCACGGGGCATCTTAGGACACATTCTGTGGAGAAA
CCCTACAAATGTGAGTTTTGTGGAAGGAGTTACAAGCAGAGAAGTTCCCT
TGAGGAGCACAAGGAGCGCTGCCGTACATTTCTTCAGAGCACTGACCCAG
GGGACACTGCAAGTGCGGAGGCAAGACACATCAAAGCAGAGATGGGAAGT
GAAAGAGCTCTCGTACTGGACAGATTAGCAAGCAATGTGGCAAAACGAAA
AAGCTCAATGCCTCAGAAATTCATTGGTGAGAAGCGCCACTGCTTTGATG
TCAACTATAATTCAAGTTACATGTATGAGAAAGAGAGTGAGCTCATACAG
ACCCGCATGATGGACCAAGCCATCAATAACGCCATCAGCTATCTTGGCGC
CGAAGCCCTGCGCCCCTTGGTCCAGACACCGCCTGCTCCCACCTCGGAGA
TGGTTCCAGTTATCAGCAGCATGTATCCCATAGCCCTCACCCGGGCTGAG
ATGTCAAACGGTGCCCCTCAAGAGCTGGAAAAGAAAAGCATCCACCTTCC
AGAGAAGAGCGTGCCTTCTGAGAGAGGCCTCTCTCCCAACAATAGTGGCC
ACGACTCCACGGACACTGACAGCAACCATGAAGAACGCCAGAATCACATC
TATCAGCAAAATCACATGGTCCTGTCTCGGGCCCGCAATGGGATGCCACT
TCTGAAGGAGGTTCCCCGCTCTTACGAACTCCTCAAGCCCCCGCCCATCT
GCCCAAGAGACTCCGTCAAAGTGATCAACAAGGAAGGGGAGGTGATGGAT
GTGTATCGGTGTGACCACTGCCGCGTCCTCTTCCTGGACTATGTGATGTT
CACGATTCACATGGGCTGCCACGGCTTCCGTGACCCTTTCGAGTGTAACA
TGTGTGGATATCGAAGCCATGATCGGTATGAGTTCTCGTCTCACATAGCC
AGAGGAGAACACAGAGCCCTGCTGAAGCTCGACGGTGGAGGCGGTTCAGG
TGGCGGAGGAAGCGGGGGAGGTGGAAGTATGGTGTCTAAAGGAGAAGAAG
ATAATATGGCTTCTCTGCCTGCTACACACGAACTGCATATTTTCGGATCT
ATTAATGGCGTGGATTTCGATATGGTGGGACAGGGAACAGGAAACCCTAA
CGATGGATACGAAGAACTGAATCTGAAGTCTACCAAAGGAGATCTGCAAT
TCTCTCCTTGGATCCTGGTCCCTCACATTGGATATGGATTCCATCAATAT
CTGCCTTACCCTGACGGAATGTCTCCTTTCCAAGCTGCTATGGTCGATGG
CTCTGGATATCAGGTCCACAGAACAATGCAATTTGAAGATGGCGCCTCTC
TGACAGTGAATTACAGATATACATACGAAGGCTCTCATATTAAAGGCGAA
GCCCAAGTGAAAGGCACAGGCTTTCCTGCCGACGGCCCTGTGATGACCAA
CTCTCTGACCGCTGCCGATTGGTGCAGATCTAAGAAGACCTACCCTAATG
ATAAAACCATTATCTCTACCTTCAAATGGTCTTACACAACAGGCAATGGC
AAGAGATACAGATCTACCGCCAGAACCACCTATACCTTTGCTAAGCCTAT
GGCTGCCAATTACCTGAAAAATCAGCCTATGTATGTGTTCAGAAAAACCG
AACTGAAACACTCTAAAACCGAACTGAATTTCAAAGAATGGCAAAAGGCT
TTCACAGATGTGATGGGAATGGATGAACTGTACAAGGGAAGCGGAGCTAC
AAATTTTAGTCTTCTTAAACAAGCCGGTGACGTGGAGGAAAATCCCGGCC
CAATGGTGAGCAAGGGCGAGGCCGTGATCAAGGAGTTCATGCGCTTCAAG
GTGCACATGGAGGGCAGCATGAACGGCCACGAGTTCGAGATCGAGGGCGA
GGGCGAGGGCCGCCCCTACGAGGGCACCCAGACCGCCAAGCTGAAGGTGA
CCAAGGGCGGCCCCCTGCCCTTCTCTTGGGACATCCTGAGCCCCCAGTTC
ATGTACGGCAGCCGCGCCTTCACCAAGCACCCCGCCGACATCCCCGACTA
CTACAAGCAGAGCTTCCCCGAGGGCTTCAAGTGGGAGCGCGTGATGAACT
TCGAGGACGGCGGCGCCGTGACCGTGACCCAGGACACCAGCCTGGAGGAC
GGCACCCTGATCTACAAGGTGAAGCTGCGCGGCACCAACTTCCCCCCCGA
CGGCCCCGTGATGCAGAAGAAGACCATGGGCTGGGAGGCCAGCACCGAGC
GCCTGTACCCCGAGGACGGCGTGCTGAAGGGCGACATCAAGATGGCCCTG
CGCCTGAAGGACGGCGGCAGATACCTGGCCGACTTCAAGACCACCTACAA
GGCCAAGAAGCCCGTGCAGATGCCCGGCGCCTACAACGTGGACCGCAAGC
TGGACATCACCAGCCACAACGAGGACTACACCGTGGTGGAGCAGTACGAG
CGCAGCGAGGGCCGCCACAGCACCGGCGGCATGGACGAGCTGTACAAGTG
A

IKZF1, IKZF2, and IKZF3 constructs were cloned into the UCOE Hygromycin expression vectors (Millipore Sigma). Reporter constructs were transfected using cationic lipid reagents into adherent HEK 293T cells and stable integrants were selected by treatment with 200 μg/mL hygromycin B. Clonal populations were obtained from the population of stable integrants either by limiting dilution or fluorescence activated cell sorting.

The clonal stable cell lines were maintained under constant 200 μg/mL hygromycin B selection while being passaged for use in the degradation assays. Flow analysis on a BD Accuri C6 showed the HEK 293T CMV-IKZF1 Clone 7 cell line to have an average Fluorescein isothiocyanate mean fluorescence intensity (FITC MFI) of 230,000 and phycoerythrin mean fluorescence intensity (PE MFI) of 33,000. HEK 293T EF1a-IKZF2 Clone 9 had an average FITC MFI of 150,000 and PE MFI of 26,000. HEK 293T EF1a-IKZF3 Clone 9 had an average FITC MFI of 400,000 and PE MFI of 60,000. The fluorescence intensity of the IKZF1/2/3-mNeonGreen (FITC channel) and mScarlet (PE channel) reporters were routinely analyzed by flow cytometry to confirm consistent expression levels between experiments.

### IKZF1/2/3 Reporter Degradation Assay

The IKZF1/IKZF2/IKZF3 degradation assays were carried out by harvesting the HEK 293T reporter cell lines and resuspending the cells in media formulated for reduced background fluorescence (FluoroBrite; Thermo Fisher). The respective cell lines were seeded at a density of 4000 cells/well into black-walled 384-well optical grade assay tissue culture plates. The cells were incubated overnight at 37° C. to allow for attachment to the assay plate. Dilutions of the compounds were prepared in DMSO from 10 mM compound stocks. The assay plates were treated with appropriate concentrations of the compounds by dispensing the DMSO dilutions in quadruplicate wells with an upper limit of 0.5% final DMSO.

After 24 hours incubation with the compounds, the assay plates were imaged on an ImageXpress Pico microscopy system (cells maintained at 37° C. during imaging) to obtain the fluorescent readouts. The assay plates were imaged in the FITC and Tetramethylrhodamine (TRITC) channels to obtain the mNeonGreen fluorescence intensity (reporter degradation data) and mScarlet fluorescence intensity (for cell segmentation). 293T-IKZF1 and 293T-IKZF3 reporter cell lines were imaged with exposures of 500 milliseconds (ms) for both FITC and TRITC channels, while the 293T-IKZF2 reporter cell line was imaged with exposures of 1000 ms for FITC and 1250 ms for TRITC. The resulting data was analyzed with Cell Reporter Xpress software using the 2-channel cell scoring analysis with a “percent positive” readout. The TRITC channel was selected for the “nuclei” segmentation with a threshold of 20 while the FITC channel was selected for the “Marker 1” segmentation and a threshold of 100 for the IKZF1 and IKZF3 reporter lines. The IKZF2 reporter line had a threshold of 120 set for the FITC channel, and 20 for TRITC. The minimum segmentation width was set to 6 micrometers and the maximum segmentation width was set to 15 micrometers for all cell lines. The DC₅₀ calculations were determined by regression to best fit four-parameter logistic curves using GraphPad Prism.

Table 3 shows results from the assays described above.

TABLE 3

| | IKZF1 | IKZF2 | IKZF3 | IKZF2 % |
|---|---|---|---|---|
| | DC50 | DC50 | DC50 | Degradation |
| # | (μM) | (μM) | (μM) | @ 1 μM |
| 1-A | >30.0 | 0.00889 | >30.0 | 83.4 |
| 1-B | >30.0 | 0.525 | >30.0 | 32.1 |
| 2-B | >30.0 | 0.120 | >30.0 | 63.1 |
| 6-B | >30.0 | 0.0299 | >30.0 | 74.9 |
| 11-B | >30.0 | 0.0207 | >30.0 | 78.2 |
| 16-B | >30.0 | 0.0226 | >30.0 | 80.6 |
| 21-B | >30.0 | 0.0382 | >30.0 | 72.5 |
| 26-B | >30.0 | 0.525 | >30.0 | 78.4 |
| 31-B | >30.0 | 0.0478 | >30.0 | 74.0 |
| 36-B | >30.0 | 0.188 | >30.0 | 49.9 |
| 46-B | >30.0 | 0.232 | >30.0 | 39.0 |
| 51-B | >30.0 | 0.321 | >30.0 | 24.0 |
| 56-B | >30.0 | 0.0111 | >30.0 | 84.8 |
| 61-B | >30.0 | 0.0260 | >30.0 | 55.8 |
| 66-B | >30.0 | 0.0132 | >30.0 | 86.4 |
| 71-B | >30.0 | 0.0283 | >30.0 | 79.3 |
| 76-B | >30.0 | 0.0286 | >30.0 | 84.6 |
| 81-B | >30.0 | 0.0965 | >30.0 | 78.0 |

Table 4 shows further results from the assays described above.

TABLE 4

| | CRBN | | | | | | |
|---|---|---|---|---|---|---|---|
| | Binding | IKZF1 | IKZF1 % | IKZF2 | IKZF2 % | IKZF3 | IKZF3 % |
| | EC50 | DC₅₀ | Degradation | DC₅₀ | Degradation | DC₅₀ | Degradation |
| # | (μM) | (μM) | @ 1 μM | (μM) | @ 1 μM | (μM) | @ 1 μM |
| 12-A | 0.052 | — | — | 0.012 | 83.1 | >30.0 | — |
| 14-A | 0.039 | >30.0 | — | 0.028 | 67.4 | >30.0 | — |
| 22-A | 0.031 | — | — | 0.011 | 75.9 | >30.0 | — |
| 24-A | 0.013 | — | — | 0.006 | 76.7 | >30.0 | — |
| 25-A | 0.004 | — | — | 0.001 | 84.4 | >30.0 | — |
| 26-A | 0.001 | — | — | 0.001 | 74.7 | >30.0 | — |
| 27-A | 0.010 | 30.0 | — | 0.008 | 84.6 | 0.11 | 44 |
| 28-A | 0.003 | >30.0 | — | 0.002 | 82.5 | 0.013 | 67.5 |
| 29-A | 0.067 | >30.0 | — | 0.011 | 83.2 | >30.0 | — |
| 30-A | 0.004 | >30.0 | — | 0.0005 | 84.4 | 0.014 | 63.4 |
| 31-A | 0.006 | >30.0 | — | 0.0001 | 88.2 | 0.061 | 26.2 |
| 32-A | 0.005 | >30.0 | — | 0.001 | 84.7 | 0.071 | 24.1 |
| 33-A | 0.007 | >30.0 | — | 0.000 | 88.4 | 0.006 | 60.8 |
| 34-A | 0.010 | >30.0 | — | 0.002 | 88 | 0.544 | 24.9 |
| 35-A | 0.008 | >30.0 | — | 0.001 | 88.2 | 8.07 | 23.5 |
| 41-A | 0.611 | — | — | 0.482 | 37.4 | 0.456 | 18.1 |
| 42-A | 0.003 | >30.0 | — | 0.001 | 87.3 | 0.021 | 31 |
| 55-A | 0.014 | >30.0 | — | 0.005 | 83.8 | >30.0 | — |
| 56-A | 0.050 | — | — | 0.030 | 60.8 | >30.0 | — |
| 57-A | 0.003 | >30.0 | — | 0.002 | 85.4 | >30.0 | — |
| 58-A | 0.003 | >30.0 | — | 0.005 | 64.5 | >30.0 | — |
| 85-B | 0.028 | >30.0 | — | 0.216 | 27.5 | — | — |
| 86-B | 0.076 | >30.0 | — | 0.189 | 22.5 | — | — |
| 87-B | 0.003 | >30.0 | — | 0.004 | 87.5 | >30.0 | — |
| 88-B | 0.010 | — | — | >30.0 | — | >30.0 | — |
| 89-B | 0.003 | — | — | 0.002 | 86.2 | >30.0 | — |
| 90-B | 0.006 | — | — | 0.001 | 87.1 | >30.0 | — |
| 91-B | 0.005 | — | — | 0.029 | 47.3 | >30.0 | — |
| 92-B | 0.012 | — | — | 0.007 | 81.7 | >30.0 | — |
| 93-B | 0.064 | — | — | 0.021 | 78 | >30.0 | — |
| 94-B | 0.029 | — | — | >30.0 | — | — | — |
| 95-B | 0.030 | — | — | 0.023 | 77.8 | >30.0 | — |
| 96-B | 0.030 | — | — | >30.0 | — | — | — |
| 97-B | 0.011 | — | — | >30.0 | — | — | — |
| 98-B | 0.055 | — | — | 0.111 | 41.3 | — | — |
| 99-B | 0.004 | — | — | 0.006 | 81.3 | >30.0 | — |
| 100-B | 0.020 | — | — | 0.005 | 80.5 | >30.0 | — |
| 101-B | 0.011 | — | — | >30.0 | — | — | — |
| 102-B | 0.015 | — | — | 0.010 | 71.1 | >30.0 | — |
| 103-B | 0.059 | — | — | 0.018 | 69.9 | >30.0 | — |
| 104-B | 0.038 | — | — | >30.0 | — | — | — |
| 105-B | 0.029 | — | — | >30.0 | — | — | — |
| 106-B | 0.006 | — | — | >30.0 | — | — | — |
| 107-B | 0.022 | — | — | >30.0 | — | — | — |
| 108-B | 0.006 | — | — | 0.006 | 74.6 | >30.0 | — |
| 109-B | 0.009 | — | — | >30.0 | — | — | — |
| 110-B | 0.032 | — | — | >30.0 | — | — | — |
| 111-B | 0.007 | — | — | 0.004 | 76 | >30.0 | — |
| 112-B | 0.020 | — | — | 0.012 | 67.1 | >30.0 | — |
| 113-B | 0.030 | — | — | 0.062 | 29.7 | — | — |
| 114-B | 0.020 | — | — | >30.0 | — | — | — |
| 115-B | 0.064 | — | — | 0.177 | 21.7 | — | — |
| 116-B | 0.006 | — | — | 0.004 | 73.7 | >30.0 | — |
| 117-B | 0.021 | — | — | 0.025 | 68.4 | >30.0 | — |
| 118-B | 0.004 | — | — | >30.0 | — | — | — |
| 119-B | 0.024 | — | — | >30.0 | — | — | — |
| 120-B | 0.005 | — | — | 0.004 | 69.9 | >30.0 | — |
| 121-B | 0.005 | — | — | >30.0 | — | — | — |
| 122-B | 0.013 | — | — | >30.0 | — | — | — |
| 123-B | 0.001 | — | — | 0.0001 | 69.6 | >30.0 | — |
| 124-B | 0.029 | — | — | >30.0 | — | >30.0 | — |
| 125-B | 0.065 | — | — | 0.083 | 50.9 | >30.0 | — |
| 126-B | 0.007 | — | — | 0.006 | 94.8 | 0.0866 | 39.5 |
| 127-B | 0.026 | — | — | 0.006 | 95.4 | 0.0633 | 39 |
| 128-B | 0.011 | — | — | 0.018 | 24.2 | >30.0 | — |
| 129-B | 0.038 | — | — | 0.020 | 42.4 | >30.0 | — |
| 130-B | 0.010 | — | — | 0.005 | 94.8 | 0.0441 | 37.4 |
| 131-B | 0.021 | — | — | 0.005 | 94.1 | 0.0414 | 34.3 |
| 132-B | 0.003 | — | — | >30.0 | — | — | — |
| 133-B | 0.010 | — | — | >30.0 | — | — | — |
| 134-B | 0.005 | — | — | 0.005 | 70.4 | >30.0 | — |
| 135-B | 0.003 | — | — | 0.013 | 77.7 | >30.0 | — |
| 136-B | 0.034 | — | — | 0.030 | 61.4 | >30.0 | — |
| 137-B | 0.038 | — | — | 0.049 | 59.3 | >30.0 | — |
| 138-B | 0.005 | — | — | >30.0 | — | >30.0 | — |
| 139-B | 0.035 | — | — | 0.033 | 62.4 | >30.0 | — |
| 140-B | 0.034 | — | — | >30.0 | — | >30.0 | — |
| 141-B | 0.037 | — | — | >30.0 | — | >30.0 | — |
| 142-B | 0.167 | — | — | 0.605 | 31.5 | >30.0 | — |
| 143-B | 0.247 | — | — | 0.581 | 20.4 | >30.0 | — |
| 144-B | > 10.0 | — | — | — | — | — | — |
| 145-B | > 10.0 | — | — | — | — | — | — |
| 146-B | 0.012 | — | — | 0.007 | 74.9 | >30.0 | — |
| 147-B | 0.022 | — | — | 0.130 | 28.6 | >30.0 | — |
| 148-B | > 10.0 | — | — | >30.0 | — | >30.0 | — |
| 149-B | > 10.0 | — | — | — | — | — | — |
| 150-B | > 10.0 | — | — | — | — | — | — |
| 151-B | 0.079 | — | — | — | — | — | — |
| 152-B | 0.003 | — | — | 0.041 | 43.9 | >30.0 | — |
| 153-B | 0.032 | — | — | 0.016 | 73.4 | >30.0 | — |
| 154-B | 0.056 | — | — | >30.0 | — | >30.0 | — |
| 155-B | 0.018 | — | — | 0.011 | 75.5 | >30.0 | — |
| 156-B | > 10.0 | — | — | — | — | — | — |
| 157-B | > 10.0 | — | — | — | — | — | — |
| 158-B | 0.088 | — | — | — | — | — | — |
| 159-B | > 10.0 | — | — | — | — | — | — |
| 160-B | > 10.0 | — | — | — | — | — | — |
| 161-B | 0.120 | >30.0 | — | 0.138 | 49.7 | >30.0 | — |
| 162-B | 0.059 | >30.0 | — | 0.022 | 70.4 | >30.0 | — |
| 163-B | 0.011 | >30.0 | — | 0.011 | 71.5 | >30.0 | — |
| 164-B | 0.015 | — | — | >30.0 | — | >30.0 | — |
| 165-B | 0.013 | — | — | 0.009 | 58.6 | >30.0 | — |
| 166-B | 0.031 | >30.0 | — | >30.0 | — | >30.0 | — |
| 167-B | 0.074 | >30.0 | — | 0.241 | 28.8 | >30.0 | — |
| 168-B | 0.048 | >30.0 | — | >30.0 | — | >30.0 | — |
| 169-B | 0.013 | >30.0 | — | 0.046 | 56.3 | >30.0 | — |
| 170-B | 0.109 | >30.0 | — | 0.066 | 54.6 | >30.0 | — |
| 171-B | > 10.0 | 30.0 | — | >30.0 | — | >30.0 | — |
| 172-B | > 10.0 | >30.0 | — | >30.0 | — | >30.0 | — |
| 173-B | 0.052 | >30.0 | — | 0.031 | 66.7 | >30.0 | — |
| 174-B | 0.036 | >30.0 | — | 0.028 | 70.3 | >30.0 | — |
| 175-B | 0.022 | >30.0 | — | >30.0 | — | >30.0 | — |
| 176-B | 0.899 | >30.0 | — | 2.100 | — | >30.0 | — |
| 177-B | 0.002 | >30.0 | — | 0.004 | 70.3 | >30.0 | — |
| 178-B | 0.039 | — | — | >30.0 | — | >30.0 | — |
| 179-B | 0.039 | >30.0 | — | 0.046 | 68.6 | >30.0 | — |
| 180-B | 0.041 | — | — | >30.0 | — | >30.0 | — |
| 181-B | 0.013 | — | — | 0.034 | <30 | >30.0 | — |
| 182-B | 0.009 | >30.0 | — | 0.005 | 78.3 | >30.0 | — |
| 183-B | 0.015 | >30.0 | — | 0.010 | 81.3 | >30.0 | — |
| 184-B | 0.017 | >30.0 | — | 0.021 | 79.6 | >30.0 | — |
| 185-B | 0.030 | >30.0 | — | 0.021 | 80.3 | >30.0 | — |
| 186-B | 0.006 | >30.0 | — | 0.005 | 79.7 | >30.0 | — |
| 187-B | 0.006 | >30.0 | — | 0.085 | 27.4 | >30.0 | — |
| 188-B | 0.044 | >30.0 | — | 0.240 | 42 | >30.0 | — |
| 189-B | 0.013 | >30.0 | — | 0.123 | 21.2 | >30.0 | — |
| 190-B | 0.044 | >30.0 | — | 0.071 | 74.3 | >30.0 | — |
| 191-B | 0.031 | >30.0 | — | >30.0 | — | >30.0 | — |
| 192-B | 0.007 | >30.0 | — | 0.123 | 25.6 | >30.0 | — |
| 193-B | 0.006 | >30.0 | — | 0.006 | 81.9 | >30.0 | — |
| 194-B | 0.017 | >30.0 | — | 0.007 | 79.8 | 0.283 | 26.6 |
| 195-B | 0.024 | >30.0 | — | >30.0 | — | >30.0 | — |
| 196-B | 0.214 | >30.0 | — | 0.601 | 17.6 | >30.0 | — |
| 197-B | 0.138 | >30.0 | — | >30.0 | — | >30.0 | — |
| 198-B | 0.010 | >30.0 | — | 0.011 | 72.3 | >30.0 | — |
| 199-B | 0.021 | >30.0 | — | 0.014 | 75 | >30.0 | — |
| 200-B | 0.020 | >30.0 | — | >30.0 | — | >30.0 | — |
| 201-B | 0.036 | >30.0 | — | 0.078 | 62.7 | >30.0 | — |
| 202-B | 0.029 | >30.0 | — | >30.0 | — | >30.0 | — |
| 203-B | 0.020 | >30.0 | — | 0.130 | 25.1 | >30.0 | — |
| 204-B | 0.037 | >30.0 | — | 0.022 | 75.4 | >30.0 | — |
| 205-B | 0.046 | >30.0 | — | 0.219 | 40.6 | >30.0 | — |
| 206-B | 0.071 | >30.0 | — | >30.0 | — | >30.0 | — |
| 207-B | 0.104 | >30.0 | — | 1.470 | 24.8 | >30.0 | — |
| 208-B | 0.076 | >30.0 | — | >30.0 | — | >30.0 | — |
| 209-B | 0.058 | >30.0 | — | 0.230 | 38.9 | >30.0 | — |
| 210-B | 0.122 | >30.0 | — | >30.0 | — | >30.0 | — |
| 211-B | 0.028 | >30.0 | — | 0.064 | 79.3 | >30.0 | — |
| 212-B | 0.057 | >30.0 | — | 0.425 | 20.3 | >30.0 | — |

### GSPT1 Degradation Assay

### Generation of Stable Cell Lines

HEK293_hGSPTl_HiBiT-tagged cells were generated using CRISPR-Cas12a technology. Briefly, ˜400,000 HEK293 cells were transiently co-transfected with precomplexed ribonuclear proteins (RNPs) consisting of 80 pmol of crRNA (IDT), 62 pmol of Cas12a protein (IDT), 3 μg of ssODN donor (IDT; Alt® modifications), 78 pmol of electroporation enhancer (IDT), and 200 ng of pMaxGFP (Lonza). The transfection was performed via nucleofection (Lonza, 4D-Nucleofector X-unit) using solution P3 and program CM-130 in a (20 L) cuvette. Five days post-nucleofection, cells were single-cell-sorted for GFP+(transfected) cells by FACs in 96-well plates and clonally selected. Clones were screened and verified for the desired modification via targeted deep sequencing using gene-specific primers with partial Illumina adapter overhangs as previously described. In brief, clonal cell pellets were harvested, lysed, and used to generate gene-specific amplicons with partial Illumina adapters in PCR #1. Amplicons were indexed in PCR #2 and pooled with other targeted amplicons for other loci to create sequence diversity. Additionally, 10% PhiX sequencing control V3 (Illumina) was added to the pooled amplicon library prior to running the sample on a Miseq Sequencer System (Illumina) to generate paired 2×250 bp reads. Samples were demultiplexed using the index sequences, fastq files were generated, and NGS analysis was performed using CRIS.py. Final clones were authenticated using the PowerPlex fusion system (Promega) and tested negative for mycoplasma by the MycoAlertTMPlus mycoplasma detection kit (Lonza).

Editing construct sequences and screening primers are outlined below (sequence from 5′ to 3′). hGSPT1Cas12acrRNA, CAGE635.GSPT1.g1: TTTCTCTGGAACCAGTTTCAGAACT; CAGE635.g1.anti.ssODN: ttcctcacagtattgtgcagggtcatcaagaaaatgcttaGCTAATCTTCTTGAACAGCCGC CAGCCGCTCACgtcCttctctggaaccagtttcagaacttttccaattgcaatggtcttacctagaaatgaaattttaa (HiBiT tag and silent blocking modifications to prevent Cas12a recutting after integration are in upper case); CAGE635.hGSPT1.DS.F: GGTTTGGCAGTAAAGCTAGTTAAT; CAGE635.hGSPT1.DS.R: GTGAA GTAGGCTTCTGCAGTC.

### GSPT1 Reporter Degradation Assay

The GSPT1 degradation assay was carried out by harvesting the HEK 293T reporter cell lines and resuspending the cells in media formulated for reduced background fluorescence (FluoroBrite; Thermo Fisher). The respective cell lines were seeded at a density of 8,000 cells/well into white-opaque 384-well optical grade assay tissue culture plates (Greiner 781080-20). The cells were incubated overnight at 37° C. to allow for attachment to the assay plate. Dilutions of the compounds were prepared in DMSO from 10 mM compound stocks. Test compounds were added in a 10-point dilution series (typically 10 μM to 100 pM) using a TECAN D300e Digital Dispenser with an upper limit of 0.5% final DMSO. Plates were centrifuged at 320×g for 2 minutes at room temperature, and subsequently incubated at 37° C.

After 24 hour incubation with the compounds, the plates were allowed to cool to room temperature for 10 minutes. 30 μL of HiBiT lytic buffer+1:50 HiBiT substrate Solution were added to each well. Plates were centrifuged at 320×g for 2 minutes at room temperature and then incubated with shaking at room temperature for 10 minutes covered with foil. Plates were read on a CLARIOstar microplate reader (BMG LabTech), measuring at 450 nm (donor emission) and 610 nm or 630 nm (acceptor emission). The DC₅₀ values were determined by regression to best fit four-parameter logistic curves using GraphPad Prism.

Table 5 shows results from the assays described above for certain compounds described herein demonstrating selectivity.

TABLE 5

| | GSPT1 | GSPT1 |
|---|---|---|
| | Degradation | Degradation |
| # | DC₅₀ (μM) | @ 1 μM ( %) |
| 14-A | >30.0 | — |
| 23-A | >30.0 | — |
| 27-A | >30.0 | — |
| 28-A | >30.0 | — |
| 29-A | >30.0 | — |
| 30-A | >30.0 | — |
| 31-A | >30.0 | — |
| 32-A | >30.0 | — |
| 33-A | >30.0 | — |
| 34-A | >30.0 | — |
| 35-A | >30.0 | — |
| 42-A | >30.0 | — |
| 55-A | >30.0 | — |
| 57-A | >30.0 | — |
| 87-B | >30.0 | — |
| 151-B | >30.0 | — |

It is contemplated that certain compounds of formula I-A, formula I-A′, formula I-B, or sub-formulae thereof described herein selectively modulate IKZF proteins over GSPT1 when compared to compounds having an oxygen-linked heterocycloalkyl or heteroaryl described in the art. Further, it is contemplated that certain compounds of formula I-A, formula I-A′, formula I-B, or sub-formulae thereof described herein selectively modulate IKZF2 over GSPT1.

### Immunoblot Analysis (KG-1 Cells)

Cells were seeded in 6-well plates (5×10\hat{ }5 cells per well). After overnight incubation, the cells were treated with indicated concentrations for 24 hr. The harvested cells were spinned down, washed with PBS, and lysed with RIPA Lysis buffer and Extraction Buffer (Thermo Scientific Cat 89900) per the manufacturer instructions. Protein quantitation was performed using the Pierce Rapid Gold BCA Protein Assay Kit (Cat A53225) using the Microplate Procedure per the manufacturer's instructions. Cell lysates were analyzed with the WES/Jess Simple Western System according to the manufacturer's instructions. The primary antibodies used were anti-IKZF2 (Abcam, ab129434, 1:25), anti-GSPT1 (Abcam, ab49878). Compounds of the disclosure tested in the assay described above induced significant degradation of IKZF2 in KG-1 cells after 24 h of treatment, with no detectable activity against GSPT1. Results were consistent with the degradation data in the IKZF2 GFP reporter and GSPT1 HiBiT-tagged cells.

1. A compound of formula I-A: 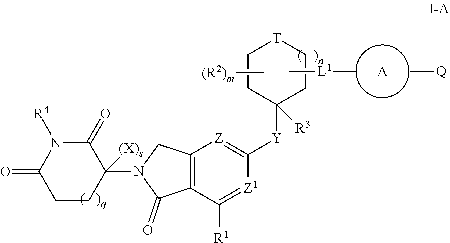
or a pharmaceutically acceptable salt, solvate, stereoisomer, or tautomer thereof, wherein:
m is zero, one, two, or three;
n is zero, one, or two;
q is one, two, or three;
s is one;
L¹ is CH₂ or a covalent bond;
Q is selected from —C(O)NR⁵R⁶ or a 4- to 10-member heterocyclic group having one or more heteroatoms independently selected from NR²⁰, O, S, and SO₂, where R²⁰ is —[C(O)]_{w}R⁷, S(O)₂R⁷, or a covalent bond linking said heterocyclic group to ring A, wherein said heterocyclic group is independently unsubstituted or substituted with C(O)R⁷, or with one to five R⁸, and where w is 0 or 1;
T is CH₂, CH(R²), C(R²)₂, or N, provided that when T is N, L¹ is attached to T;
X is hydrogen, deuterium, or fluoro;
Y is a covalent bond, —O—, or —NR—;
Z and Z¹ are each independently CR¹ or N;
ring A is phenyl or a 4- to 7-membered nitrogen containing heterocyclyl, wherein ring A is independently unsubstituted or substituted with one to five R⁸;
R is hydrogen or C₁-C₄ alkyl;
each R¹ is independently hydrogen, halo, cyano, hydroxy, —N(R⁹)₂, C₁-C₄ alkyl independently unsubstituted or substituted with from one to three R¹⁰, or C₁-C₆ alkoxy independently unsubstituted or substituted with from one to three R¹⁰; or
when Z¹ is CR¹, then two adjacent R¹ together with the carbon atoms to which they are attached form a C₃-C₇ cycloalkyl, a C₆-C₁₀ aryl, a 4- to 7-membered heterocycloalkenyl, or a 5- to 6-membered heteroaryl, wherein each of said cycloalkyl, heterocycloalkenyl, aryl, and heteroaryl are independently substituted with one to three R¹¹;
each R² is independently selected from halo, cyano, hydroxy, —N(R⁹)₂, C₁-C₄ alkyl independently unsubstituted or substituted with from one to three R¹⁰, and C₁-C₄ alkoxy independently unsubstituted or substituted with from one to three R¹⁰;
R³ is selected from the group consisting of hydrogen, amino, halo, cyano, hydroxy, C₁-C₆ alkyl, and C₁-C₆ haloalkyl;
R⁴ is hydrogen or —CH₂—OR¹²;
R⁵ and R⁶, together with the nitrogen to which they are attached, form a 4- to 7-membered heterocyclyl, wherein said heterocyclyl is independently unsubstituted or substituted with one to five R⁸;
R⁷ is hydrogen, C₁-C₆ alkyl, C₁-C₆ alkoxy, C₃-C₇ cycloalkyl, C₂-C₆ alkenyl, C₂-C₆ alkynyl, 4- to 7-membered heterocyclyl, C₆-C₁₀ aryl, 5- to 10-membered heteroaryl, or —N(R⁸)₂; wherein said alkyl, alkoxy, cycloalkyl, alkenyl, alkynyl, heterocyclyl, aryl, or heteroaryl is further independently unsubstituted or substituted with one to five R⁸;
each R⁸ is independently halo, cyano, hydroxy, —SH, —NH₂, —NO₂, —SF₅, C₁-C₆ alkyl, C₂-C₆ alkenyl, C₂-C₆ alkynyl, C₁-C₆ haloalkyl, C₁-C₆ alkoxy, C₁-C₆ haloalkoxy, C₃-C₁₀ cycloalkyl, heterocyclyl, aryl, or heteroaryl;
each R⁹ is independently H or C₁-C₄ alkyl independently unsubstituted or substituted with from 1 to 3 substituents selected from halo, cyano, hydroxy, and C₁-C₄ alkoxy, or two R⁹ together with the nitrogen atom to which they are attached form a 3- to 7-membered heterocycloalkyl or 5- to 6-membered heteroaryl;
each R¹⁰ is independently —N(R⁹)₂, halo, cyano, hydroxy, or C₁-C₄ alkoxy;
each R¹¹ is independently —N(R⁹)₂, halo, cyano, hydroxy, or oxo;
R¹² is —C(O)—R¹³ or —P(O)(OR¹⁴)₂;
R¹³ is C₁-C₄ alkyl or C₁-C₄ alkoxy; and
each R¹⁴ is independently H or C₁-C₄ alkyl.

2. The compound of claim 1, wherein Q is

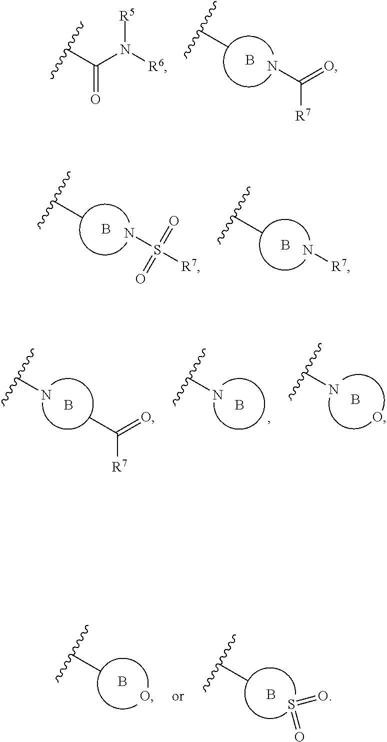

3. The compound of claim 1, wherein T is N and L¹ is CH₂.

4. The compound of claim 3, wherein ring A is phenyl or piperidinyl.

5. The compound of claim 4, wherein ring A is phenyl and Q is

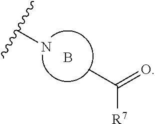

6.-7. (canceled)

8. The compound of claim 1, wherein the compound is represented by formula IIB-A: 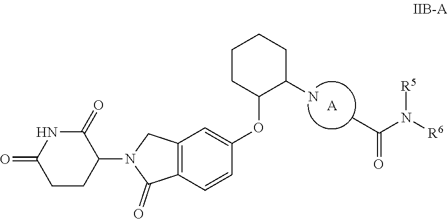
or a pharmaceutically acceptable salt, solvate, stereoisomer, or tautomer thereof.

9. (canceled)

10. The compound of claim 8, wherein R⁵ and R⁶, together with the nitrogen to which they are attached, form a 4- to 7-membered heterocyclyl group optionally having an additional 1 to 2 heteroatoms selected from N, NR, O, and S, wherein said heterocyclyl is independently unsubstituted or substituted with one to five R⁸.

11. The compound of claim 8, wherein NR⁵R⁶ is

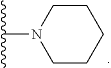 [SMILES: *N1CCCCC1]

12.-13. (canceled)

14. The compound of claim 1, wherein the compound is represented by formula IIIB-A: 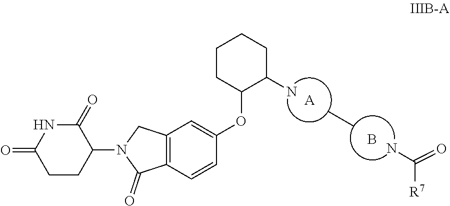
or a pharmaceutically acceptable salt, solvate, stereoisomer, or tautomer thereof.

15.-16. (canceled)

17. The compound of claim 14, wherein R⁷ is C₁-C₆ alkyl, C₁-C₆ alkoxy, C₃-C₇ cycloalkyl, 4- to 7-membered heterocyclyl, or C₆-C₁₀ aryl, wherein said alkyl, cycloalkyl, heterocyclyl, or aryl is further independently unsubstituted or substituted with 1 to 3 substituents selected from halo, cyano, C₁-C₄ alkyl, and C₃-C₇ cycloalkyl.

18.-19. (canceled)

20. The compound of claim 1, wherein the compound is represented by formula IVA-A: 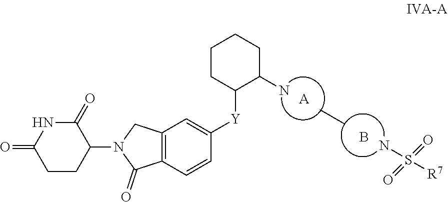
or a pharmaceutically acceptable salt, solvate, stereoisomer, or tautomer thereof.

21. (canceled)

22. The compound of claim 20, wherein R⁷ is C₃-C₇ cycloalkyl independently unsubstituted or substituted with 1 to 3 substituents selected from halo, cyano, C₁-C₄ alkyl, and C₃-C₇ cycloalkyl.

23. (canceled)

24. The compound of claim 1, wherein the compound is represented by formula VA-A: 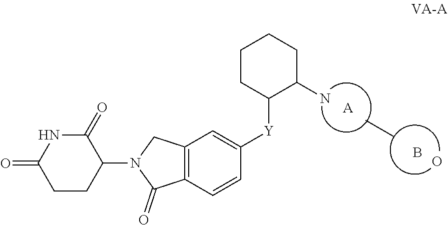
or a pharmaceutically acceptable salt, solvate, stereoisomer, or tautomer thereof.

25. (canceled)

26. The compound of claim 24, wherein R⁸ is hydroxy.

27. (canceled)

28. The compound of claim 1, wherein the compound is represented by formula VIA-A: 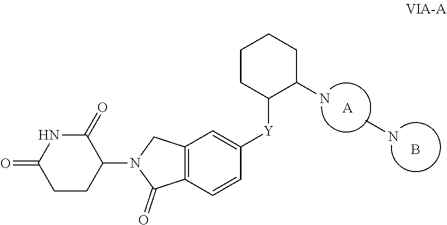
or a pharmaceutically acceptable salt, solvate, stereoisomer, or tautomer thereof.

29. (canceled)

30. The compound of claim 28, wherein R⁸ is C₁-C₆ alkyl or halo.

31. (canceled)

32. The compound of claim 1, wherein the compound is represented by formula VIIA-A: 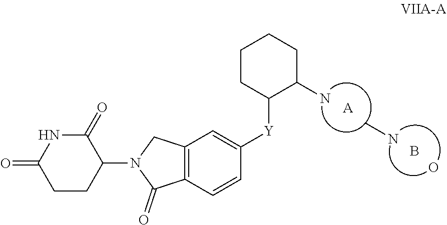
or a pharmaceutically acceptable salt, solvate, stereoisomer, or tautomer thereof.

33.-34. (canceled)

35. The compound of claim 1, wherein the compound is represented by formula VIIIA-A: 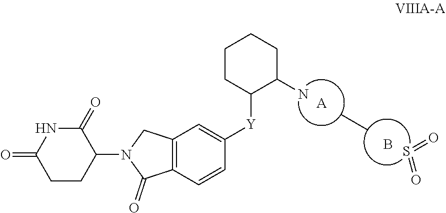
or a pharmaceutically acceptable salt, solvate, stereoisomer, or tautomer thereof.

36.-37. (canceled)

38. The compound of claim 1, wherein the compound is represented by formula IXA-A: 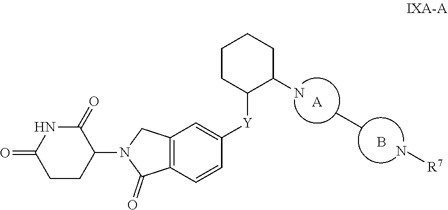
or a pharmaceutically acceptable salt, solvate, stereoisomer, or tautomer thereof.

39. (canceled)

40. The compound of claim 38, wherein R⁷ is hydrogen or C₁-C₆ alkyl.

41. A compound of formula I-B: 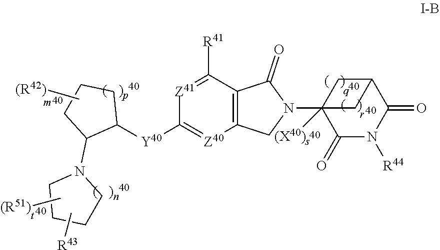
or a pharmaceutically acceptable salt, solvate, stereoisomer, and/or tautomer thereof,
wherein:
m⁴⁰, n⁴⁰ and p⁴⁰ are independently zero, one, two, or three;
q⁴⁰ is one, two or three;
r⁴⁰ is zero, one, or two;
s⁴⁰ is zero when r⁴⁰ is not zero and is one when r⁴⁰ is zero;
t⁴⁰ is zero or one;
X⁴⁰ is hydrogen, deuterium, or fluoro;
Y⁴⁰ is oxygen or NR⁴⁰ where R⁴⁰ is hydrogen or C₁-C₄ alkyl;
Z⁴⁰ and Z⁴¹ are each independently CR⁴¹ or N;
each R⁴¹ is independently selected from hydrogen, amino, (C₁-C₄ alkyl)amino unsubstituted or substituted with from one to three R⁴⁵ substituents, di-(C₁-C₄ alkyl)amino unsubstituted or substituted with from one to three R⁴⁵ substituents on each alkyl group, cyano, halo, hydroxyl, C₁-C₄ alkyl unsubstituted or substituted with from one to three R⁴⁵ substituents, and C₁-C₄ alkoxy unsubstituted or substituted with from one to three R⁴⁵ substituents; or
when Z⁴¹ is CR⁴¹, then two adjacent R⁴¹ together with the carbon atoms to which they are attached form a C₃-C₇ cycloalkyl, a C₆-C₁₀ aryl, a 4- to 7-membered heterocycloalkenyl having from one to three heteroatoms selected from oxygen, nitrogen, or sulfur, or a 5- to 6-membered heteroaryl having 1 to 3 heteroatoms selected from oxygen, nitrogen, or sulfur wherein each of said cycloalkyl, heterocycloalkenyl, aryl, and heteroaryl are independently substituted with one to three R⁴⁶ groups;
each R⁴² is independently selected from cyano, halo, hydroxyl, amino, C₁-C₄ alkylamino unsubstituted or substituted with from one to three R⁴⁵ substituents, di-(C₁-C₄ alkyl)amino unsubstituted or substituted with from one to three R⁴⁵ substituents on each alkyl group, C₁-C₄ alkyl unsubstituted or substituted with from one to three R⁴⁵ substituents, and C₁-C₄ alkoxy unsubstituted or substituted with from one to three R⁴⁵ substituents;
R⁴³ is C₅-C₁₀ heteroaryl having from 1 to 3 heteroatoms selected from O, N, and/or S and unsubstituted or substituted with 1 to 3 R⁴⁷ substituents;
R⁴⁴ is selected from hydrogen and —CH₂—OR⁴⁸ where R⁴⁸ is C(O)—R⁴⁹ or —P(O)(OR⁵⁰)₂, where R⁴⁹ is C₁-C₄ alkyl or C₁-C₄ alkoxy, and where each of R⁵⁰ is independently H or C₁-C₄ alkyl;
each R⁴⁵ is independently amino, (C₁-C₄ alkyl)amino, di-(C₁-C₄ alkyl)amino, cyano, halo, hydroxyl, or C₁-C₄ alkoxy;
each R⁴⁶ is independently selected from amino, (C₁-C₄ alkyl)amino, di-(C₁-C₄ alkyl)amino, cyano, halo, hydroxyl, and oxo;
each R⁴⁷ is independently selected from amino, C₁-C₄ alkyl unsubstituted or substituted with 1 to 3 halo, C₁-C₄ alkoxy, (C₁-C₄ alkyl)amino, di-(C₁-C₄ alkyl)amino, cyano, halo, hydroxyl, nitro, C₅-C₆ heteroaryl having from 1 to 3 heteroatoms selected from O, NR⁴⁰, and/or S, C₃-C₇ cycloalkyl, and —C(O)CH₃; and
R⁵¹ is hydroxyl, cyano, halo, or C₁-C₄ alkyl unsubstituted or substituted with 1 to 3 halo.

42.-62. (canceled)

63. A compound, or a pharmaceutically acceptable salt, solvate, stereoisomer, or tautomer thereof,
wherein the compound is selected from:

| # | Structure | Nomenclature |
|---|---|---|
| 1-A | 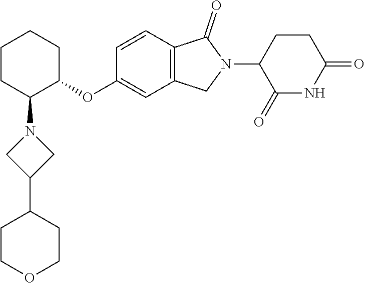 [SMILES: O=C1CCC(N2Cc3cc(O[C@H]4CCCC[C@@H]4N4CC(C5CCOCC5)C4)ccc3C2=O)C(=O)N1] | 3-(1-oxo-5-(((1S,2S)-2-(3- (tetrahydro-2H-pyran-4-yl)- azetidin-1-yl)cyclohexyl)- oxy)isoindolin-2-yl)piperi- dine-2,6-dione |
| 2-A | 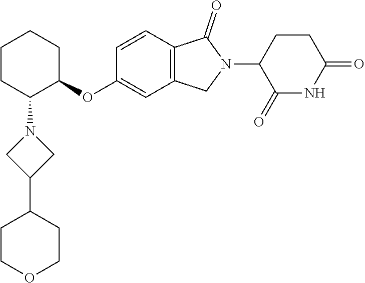 [SMILES: O=C1CCC(N2Cc3cc(O[C@@H]4CCCC[C@H]4N4CC(C5CCOCC5)C4)ccc3C2=O)C(=O)N1] | 3-(1-oxo-5-(((1R,2R)-2-(3- (tetrahydro-2H-pyran-4-yl)- azetidin-1-yl)cyclohexyl)- oxy)isoindolin-2-yl)piperi- dine-2,6-dione |
| 3-A | 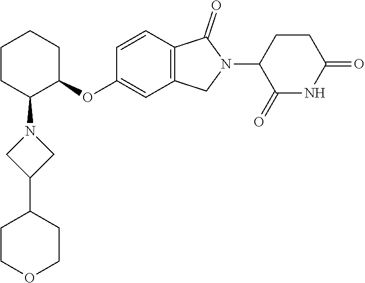 [SMILES: O=C1CCC(N2Cc3cc(O[C@@H]4CCCC[C@@H]4N4CC(C5CCOCC5)C4)ccc3C2=O)C(=O)N1] | 3-(1-oxo-5-(((1R,2S)-2-(3- (tetrahydro-2H-pyran-4-yl)- azetidin-1-yl)cyclohexyl)- oxy)isoindolin-2-yl)piperi- dine-2,6-dione |
| 4-A | 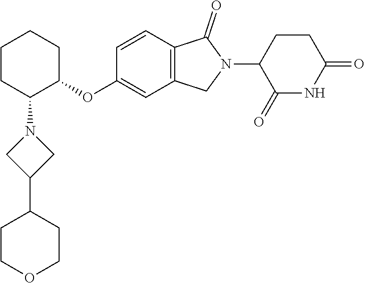 [SMILES: O=C1CCC(N2Cc3cc(O[C@H]4CCCC[C@H]4N4CC(C5CCOCC5)C4)ccc3C2=O)C(=O)N1] | 3-(1-oxo-5-(((1S,2R)-2-(3- (tetrahydro-2H-pyran-4-yl)- azetidin-1-yl)cyclohexyl)- oxy)isoindolin-2-yl)piperi- dine-2,6-dione |
| 5-A | 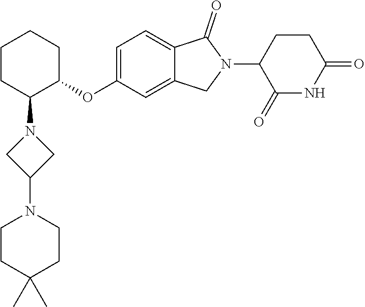 [SMILES: CC1(C)CCN(C2CN([C@H]3CCCC[C@@H]3Oc3ccc4c(c3)CN(C3CCC(=O)NC3=O)C4=O)C2)CC1] | 3-(5-(((1S,2S)-2-(3-(4,4- dimethylpiperidin-1-yl)- azetidin-1-yl)cyclohexyl)- oxy)-1-oxoisoindolin-2-yl)- piperidine-2,6-dione |
| 6-A | 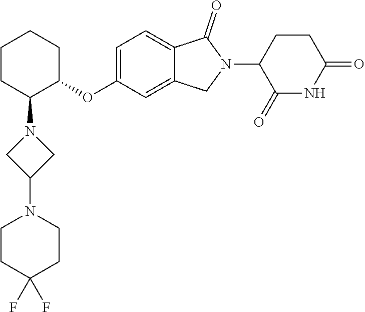 [SMILES: O=C1CCC(N2Cc3cc(O[C@H]4CCCC[C@@H]4N4CC(N5CCC(F)(F)CC5)C4)ccc3C2=O)C(=O)N1] | 3-(5-(((1S,2S)-2-(3-(4,4- difluoropiperidin-1-yl)- azetidin-1-yl)cyclohexyl)- oxy)-1-oxo-isoindolin-2-yl)- piperidine-2,6-dione |
| 7-A | 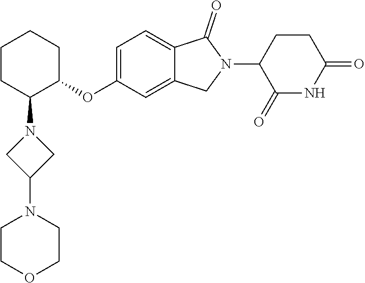 [SMILES: O=C1CCC(N2Cc3cc(O[C@H]4CCCC[C@@H]4N4CC(N5CCOCC5)C4)ccc3C2=O)C(=O)N1] | 3-(5-(((1S,2S)-2-(3-morpho- linoazetidin-1-yl)cyclohex- yl)oxy)-1-oxoisoindolin-2- yl)piperidine-2,6-dione |
| 8-A | 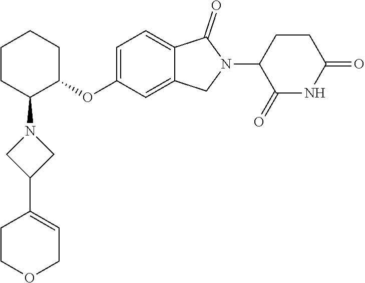 [SMILES: O=C1CCC(N2Cc3cc(O[C@H]4CCCC[C@@H]4N4CC(C5=CCOCC5)C4)ccc3C2=O)C(=O)N1] | 3-(5-(((1S,2S)-2-(3-(3,6- dihydro-2H-pyran-4-yl)- azetidin-1-yl)cyclohexyl)- oxy)-1-oxoisoindolin-2-yl)- piperidine-2,6-dione |
| 9-A | 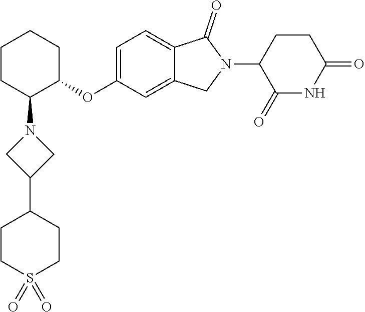 [SMILES: O=C1CCC(N2Cc3cc(O[C@H]4CCCC[C@@H]4N4CC(C5CCS(=O)(=O)CC5)C4)ccc3C2=O)C(=O)N1] | 3-(5-(((1S,2S)-2-(3-(1,1- dioxidotetrahydro-2H-thio- pyran-4-yl)-azetidin-1-yl)- cyclohexyl)oxy)-1-oxoiso- indolin-2-yl)piperidine-2,6- dione |
| 10-A | 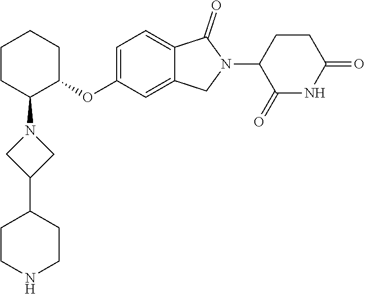 [SMILES: O=C1CCC(N2Cc3cc(O[C@H]4CCCC[C@@H]4N4CC(C5CCNCC5)C4)ccc3C2=O)C(=O)N1] | 3-(1-oxo-5-(((1S,2S)-2-(3- (piperidin-4-yl)azetidin-1- yl)cyclohexyl)oxy)isoin- dolin-2-yl)piperidine-2,6- dione |
| 11-A | 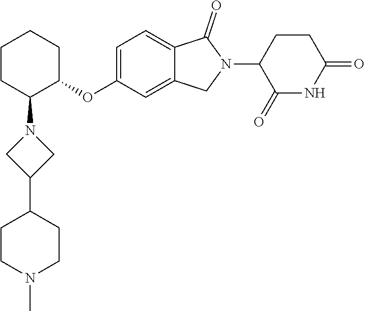 [SMILES: CN1CCC(C2CN([C@H]3CCCC[C@@H]3Oc3ccc4c(c3)CN(C3CCC(=O)NC3=O)C4=O)C2)CC1] | 3-(5-(((1S,2S)-2-(3-(1-meth- ylpiperidin-4-yl)azetidin-1- yl)cyclohexyl)oxy)-1-oxo- isoindolin-2-yl)piperidine- 2,6-dione |
| 12-A | 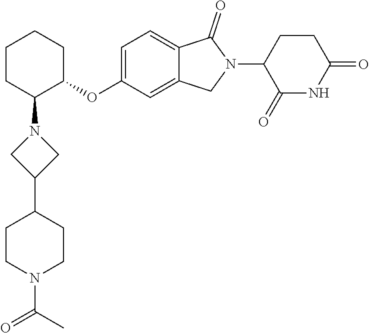 [SMILES: CC(=O)N1CCC(C2CN([C@H]3CCCC[C@@H]3Oc3ccc4c(c3)CN(C3CCC(=O)NC3=O)C4=O)C2)CC1] | 3-(5-(((1S,2S)-2-(3-(1-acet- ylpiperidin-4-yl)azetidin-1- yl)cyclohexyl)oxy)-1-oxo- isoindolin-2-yl)piperidine- 2,6-dione |
| 13-A | 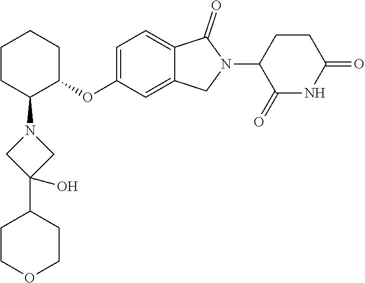 [SMILES: O=C1CCC(N2Cc3cc(O[C@H]4CCCC[C@@H]4N4CC(O)(C5CCOCC5)C4)ccc3C2=O)C(=O)N1] | 3-(5-(((1S,2S)-2-(3-hydroxy- 3-(tetrahydro-2H-pyran-4- yl)azetidin-1-yl)cyclohex- yl)oxy)-1-oxoisoindolin-2- yl)piperidine-2,6-dione |
| 14-A | 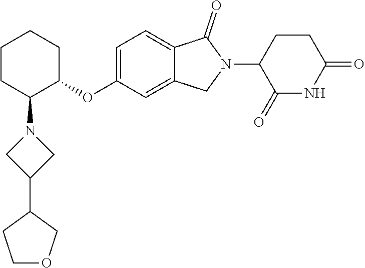 [SMILES: O=C1CCC(N2Cc3cc(O[C@H]4CCCC[C@@H]4N4CC(C5CCOC5)C4)ccc3C2=O)C(=O)N1] | 3-(1-oxo-5-(((1S,2S)-2-(3- (tetrahydrofuran-3-yl)azeti- din-1-yl)cyclohexyl)oxy)- isoindolin-2-yl)piperidine- 2,6-dione |
| 15-A | 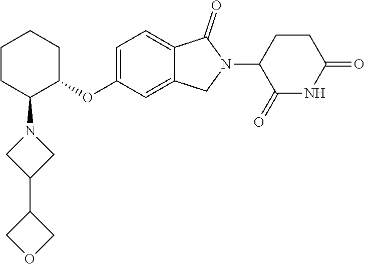 [SMILES: O=C1CCC(N2Cc3cc(O[C@H]4CCCC[C@@H]4N4CC(C5COC5)C4)ccc3C2=O)C(=O)N1] | 3-(5-(((1S,2S)-2-(3-(oxetan- 3-yl)azetidin-1-yl)cyclohex- yl)oxy)-1-oxoisoindolin-2- yl)piperidine-2,6-dione |
| 16-A | 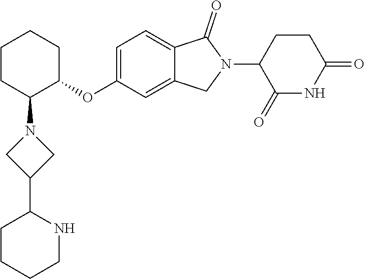 [SMILES: O=C1CCC(N2Cc3cc(O[C@H]4CCCC[C@@H]4N4CC(C5CCCCN5)C4)ccc3C2=O)C(=O)N1] | 3-(1-oxo-5-(((1S,2S)-2-(3- (piperidin-2-yl)azetidin-1- yl)cyclohexyl)oxy)isoin- dolin-2-yl)piperidine-2,6- dione |
| 17-A | 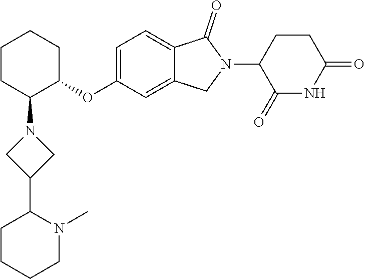 [SMILES: CN1CCCCC1C1CN([C@H]2CCCC[C@@H]2Oc2ccc3c(c2)CN(C2CCC(=O)NC2=O)C3=O)C1] | 3-(5-(((1S,2S)-2-(3-(1-meth- ylpiperidin-2-yl)azetidin-1- yl)cyclohexyl)oxy)-1-oxo- isoindolin-2-yl)piperidine- 2,6-dione |
| 18-A | 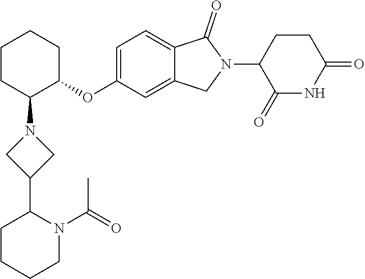 [SMILES: CC(=O)N1CCCCC1C1CN([C@H]2CCCC[C@@H]2Oc2ccc3c(c2)CN(C2CCC(=O)NC2=O)C3=O)C1] | 3-(5-(((1S,2S)-2-(3-(1-acet- ylpiperidin-2-yl)azetidin-1- yl)cyclohexyl)oxy)-1-oxo- isoindolin-2-yl)piperidine- 2,6-dione |
| 19-A | 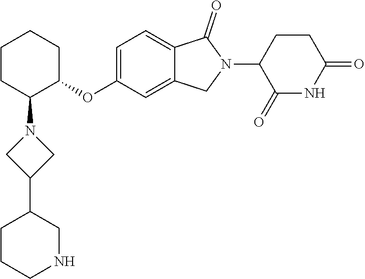 [SMILES: O=C1CCC(N2Cc3cc(O[C@H]4CCCC[C@@H]4N4CC(C5CCCNC5)C4)ccc3C2=O)C(=O)N1] | 3-(1-oxo-5-(((1S,2S)-2-(3- (piperidin-3-yl)azetidin-1- yl)cyclohexyl)oxy)isoindolin- 2-yl)-piperidine-2,6-dione |
| 20-A | 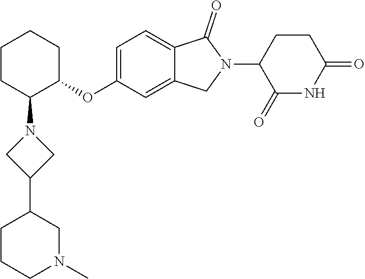 [SMILES: CN1CCCC(C2CN([C@H]3CCCC[C@@H]3Oc3ccc4c(c3)CN(C3CCC(=O)NC3=O)C4=O)C2)C1] | 3-(5-(((1S,2S)-2-(3-(1-meth- ylpiperidin-3-yl)azetidin-1- yl)cyclohexyl)oxy)-1-oxo- isoindolin-2-yl)piperidine- 2,6-dione |
| 21-A | 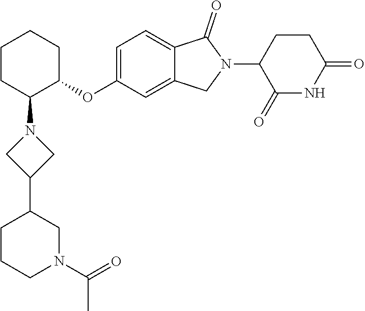 [SMILES: CC(=O)N1CCCC(C2CN([C@H]3CCCC[C@@H]3Oc3ccc4c(c3)CN(C3CCC(=O)NC3=O)C4=O)C2)C1] | 3-(5-(((1S,2S)-2-(3-(1-acet- ylpiperidin-3-yl)azetidin-1- yl)cyclohexyl)oxy)-1-oxo- isoindolin-2-yl)piperidine- 2,6-dione |
| 22-A | 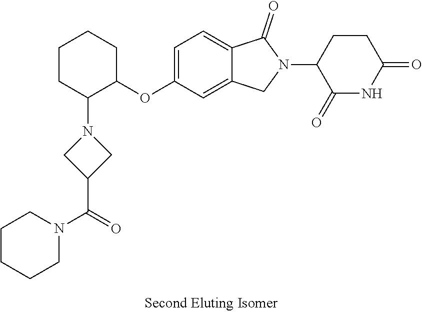 [SMILES: O=C1CCC(N2Cc3cc(OC4CCCCC4N4CC(C(=O)N5CCCCC5)C4)ccc3C2=O)C(=O)N1] | 3-(1-oxo-5-((2-(3-(piperi- dine-1-carbonyl)azetidin-1- yl)cyclohexyl)oxy)isoindolin- 2-yl)piperidine-2,6-dione |
| 23-A | 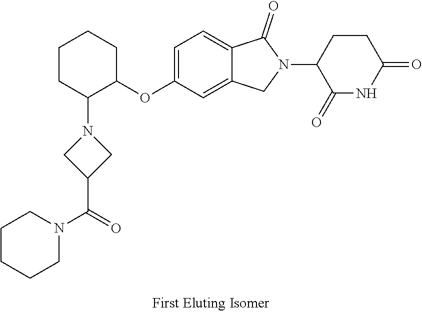 [SMILES: O=C1CCC(N2Cc3cc(OC4CCCCC4N4CC(C(=O)N5CCCCC5)C4)ccc3C2=O)C(=O)N1] | 3-(1-oxo-5-((2-(3-(piperi- dine-1-carbonyl)azetidin-1- yl)cyclohexyl)oxy)isoindolin- 2-yl)piperidine-2,6-dione |
| 24-A | 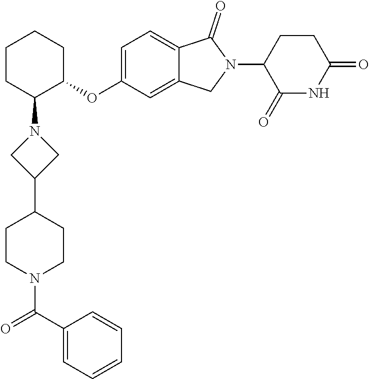 [SMILES: O=C1CCC(N2Cc3cc(O[C@H]4CCCC[C@@H]4N4CC(C5CCN(C(=O)c6ccccc6)CC5)C4)ccc3C2=O)C(=O)N1] | 3-(5-{[(1S,2S)-2-13-(1-benz- oylpiperidin-4-yl)azetidin-1- yl]cyclohexyl]oxy}-1-oxo- 2,3-dihydro-1H-isoindol-2- yl)piperidine-2,6-dione |
| 25-A | 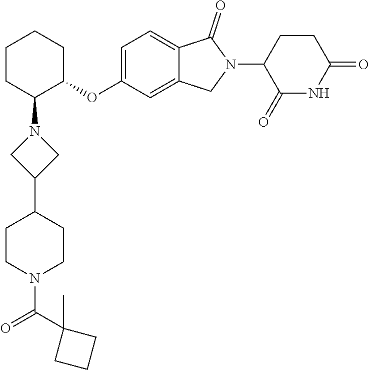 [SMILES: CC1(C(=O)N2CCC(C3CN([C@H]4CCCC[C@@H]4Oc4ccc5c(c4)CN(C4CCC(=O)NC4=O)C5=O)C3)CC2)CCC1] | 3-(5-{[(1S,2S)-2-{3-[1-(1- methylcyclobutanecarbon- yl)piperidin-4-yl]azetidin-1- yl}cyclohexyl]oxy}-1-oxo- 2,3-dihydro-1H-isoindol-2- yl)piperidine-2,6-dione |
| 26-A | 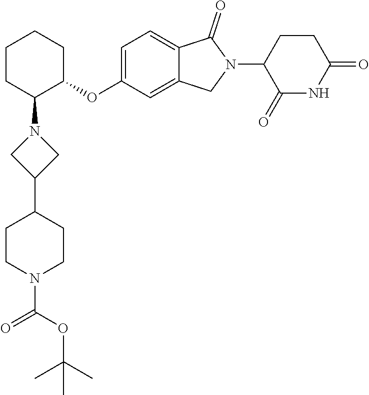 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1] | tert-butyl 4-{1-[(1S,2S)-2- {[2-(2,6-dioxopiperidin-3- yl)-1-oxo-2,3-dihydro-1H- isoindol-5-yl]oxy}cyclohex- yl]azetidin-3-yl}piperidine- 1-carboxylate |
| 27-A | 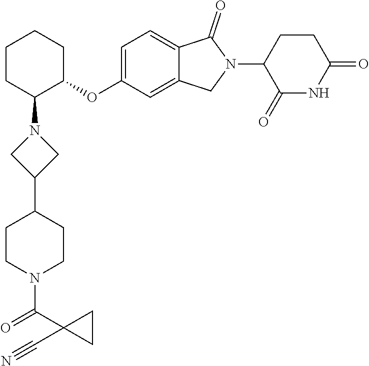 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1] | 1-(4-(1-((1S,2S)-2-((2-(2,6- dioxopiperidin-3-yl)-1-oxo- isoindolin-5-yl)oxy)cyclo- hexyl)azetidin-3-yl)piperi- dine-1-carbonyl)cyclopro- pane-1-carbonitrile |
| 28-A | 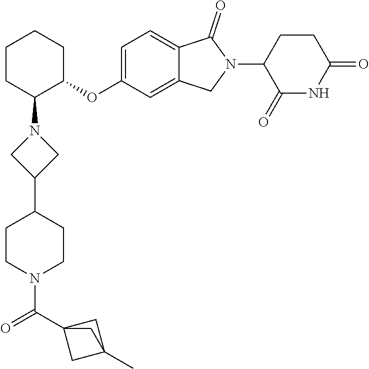 [SMILES: CC12CC(C(=O)N3CCC(C4CN([C@H]5CCCC[C@@H]5Oc5ccc6c(c5)CN(C5CCC(=O)NC5=O)C6=O)C4)CC3)(C1)C2] | 3-(5-(((1S,2S)-2-(3-(1-(3- methylbicyclo[1.1.1]pentane- 1-carbonyl)piperidin-4-yl)- azetidin-1-yl)cyclohexyl)- oxy)-1-oxoisoindolin-2-yl)- piperidine-2,6-dione |
| 29-A | 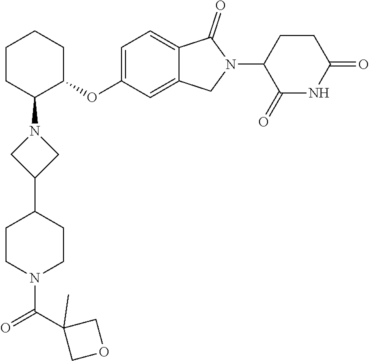 [SMILES: CC1(C(=O)N2CCC(C3CN([C@H]4CCCC[C@@H]4Oc4ccc5c(c4)CN(C4CCC(=O)NC4=O)C5=O)C3)CC2)COC1] | 3-(5-(((1S,2S)-2-(3-(1-(3- methyloxetane-3-carbonyl)- piperidin-4-yl)azetidin-1- yl)cyclohexyl)oxy)-1-oxo- isoindolin-2-yl)piperidine- 2,6-dione |
| 30-A | 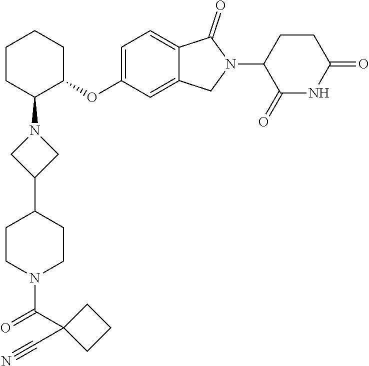 [SMILES: N#CC1(C(=O)N2CCC(C3CN([C@H]4CCCC[C@@H]4Oc4ccc5c(c4)CN(C4CCC(=O)NC4=O)C5=O)C3)CC2)CCC1] | 1-(4-(1-((1S,2S)-2-((2-(2,6- dioxopiperidin-3-yl)-1-oxo- isoindolin-5-yl)oxy)cyclohex- yl)azetidin-3-yl)piperidine-1- carbonyl)cyclobutane-1- carbonitrile |
| 31-A | 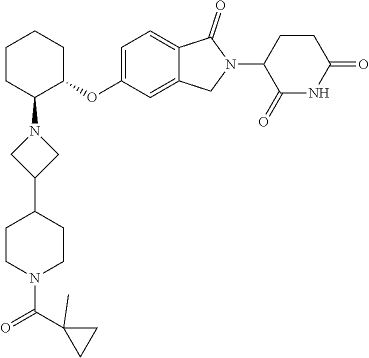 [SMILES: CC1(C(=O)N2CCC(C3CN([C@H]4CCCC[C@@H]4Oc4ccc5c(c4)CN(C4CCC(=O)NC4=O)C5=O)C3)CC2)CC1] | 3-(5-(((1S,2S)-2-(3-(1-(1- methylcyclopropane-1- carbonyl)piperidin-4-yl)- azetidin-1-yl)cyclohexyl)- oxy)-1-oxoisoindolin-2-yl)- piperidine-2,6-dione |
| 32-A | 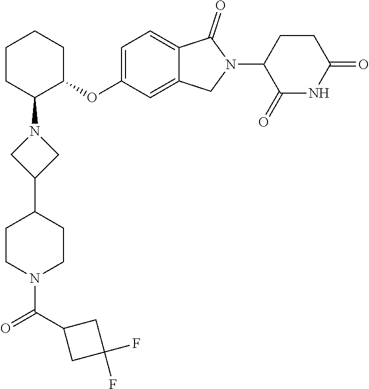 [SMILES: O=C1CCC(N2Cc3cc(O[C@H]4CCCC[C@@H]4N4CC(C5CCN(C(=O)C6CC(F)(F)C6)CC5)C4)ccc3C2=O)C(=O)N1] | 3-(5-(((1S,2S)-2-(3-(1- (3,3-difluorocyclobutane- 1-carbonyl)piperidin-4-yl)- azetidin-1-yl)cyclohexyl)- oxy)-1-oxoisoindolin-2-yl)- piperidine-2,6-dione |
| 33-A | 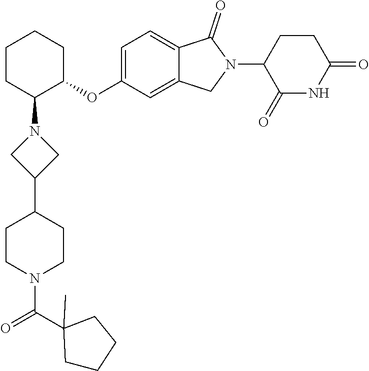 [SMILES: CC1(C(=O)N2CCC(C3CN([C@H]4CCCC[C@@H]4Oc4ccc5c(c4)CN(C4CCC(=O)NC4=O)C5=O)C3)CC2)CCCC1] | 3-(5-(((1S,2S)-2-(3-(1-(1- methylcyclopentane-1- carbonyl)piperidin-4-yl)- azetidin-1-yl)cyclohexyl)- oxy)-1-oxoisoindolin-2-yl)- piperidine-2,6-dione |
| 34-A | 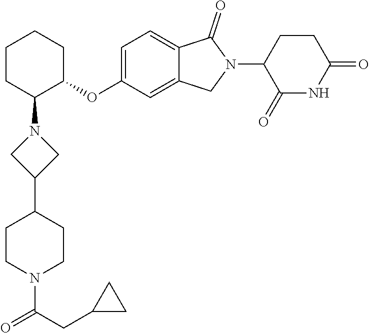 [SMILES: O=C1CCC(N2Cc3cc(O[C@H]4CCCC[C@@H]4N4CC(C5CCN(C(=O)CC6CC6)CC5)C4)ccc3C2=O)C(=O)N1] | 3-(5-(((1S,2S)-2-(3-(1-(2- cyclopropylacetyl)piperidin- 4-yl)azetidin-1-yl)cyclohex- yl)oxy)-1-oxoisoindolin-2- yl)piperidine-2,6-dione |
| 35-A | 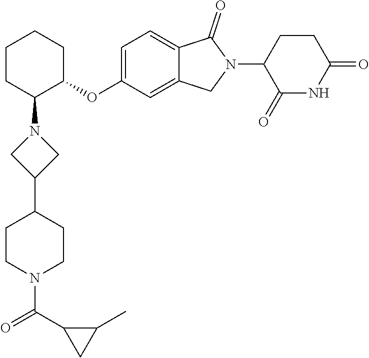 [SMILES: CC1CC1C(=O)N1CCC(C2CN([C@H]3CCCC[C@@H]3Oc3ccc4c(c3)CN(C3CCC(=O)NC3=O)C4=O)C2)CC1] | 3-(5-(((1S,2S)-2-(3-(1-(2- methylcyclopropane-1- carbonyl)piperidin-4-yl)- azetidin-1-yl)cyclohexyl)- oxy)-1-oxoisoindolin-2-yl)- piperidine-2,6-dione |
| 36-A | 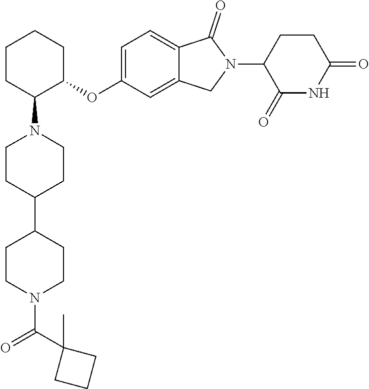 [SMILES: CC1(C(=O)N2CCC(C3CCN([C@H]4CCCC[C@@H]4Oc4ccc5c(c4)CN(C4CCC(=O)NC4=O)C5=O)CC3)CC2)CCC1] | 3-(5-(((1S,2S)-2-(1′-(1-meth- ylcyclobutane-1-carbonyl)- [4,4′-bipiperidin]-1-yl)cyclo- hexyl)oxy)-1-oxoisoindolin- 2-yl)piperidine-2,6-dione |
| 38-A | 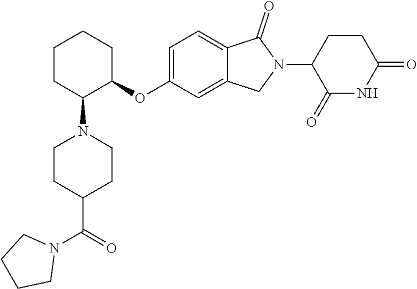 [SMILES: O=C1CCC(N2Cc3cc(O[C@@H]4CCCC[C@@H]4N4CCC(C(=O)N5CCCC5)CC4)ccc3C2=O)C(=O)N1] | 3-(1-oxo-5-(((1R,2S)-2-(4- (pyrrolidine-1-carbonyl)- piperidin-1-yl)cyclohexyl)- oxy)isoindolin-2-yl)piperi- dine-2,6-dione |
| 39-A | 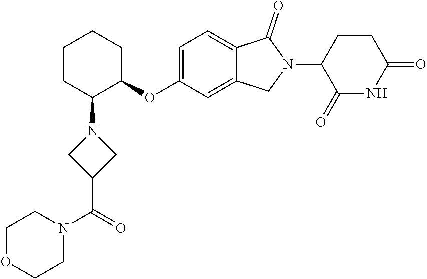 [SMILES: O=C1CCC(N2Cc3cc(O[C@@H]4CCCC[C@@H]4N4CC(C(=O)N5CCOCC5)C4)ccc3C2=O)C(=O)N1] | 3-(5-(((1R,2S)-2-(3-(morph- oline-4-carbonyl)azetidin-1- yl)cyclohexyl)oxy)-1-oxoiso- indolin-2-yl)piperidine-2,6- dione |
| 40-A | 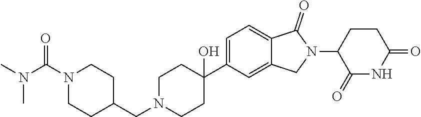 [SMILES: CN(C)C(=O)N1CCC(CN2CCC(O)(c3ccc4c(c3)CN(C3CCC(=O)NC3=O)C4=O)CC2)CC1] | 4-((4-(2-(2,6-dioxopiperidin- 3-yl)-1-oxoisoindolin-5-yl)- 4-hydroxypiperidin-1-yl)- methyl)-N,N-dimethylpiperi- dine-1-carboxamide |
| 41-A | 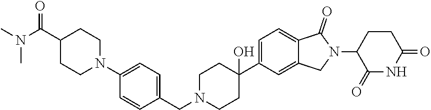 [SMILES: CN(C)C(=O)C1CCN(c2ccc(CN3CCC(O)(c4ccc5c(c4)CN(C4CCC(=O)NC4=O)C5=O)CC3)cc2)CC1] | 1-(4-((4-(2-(2,6-dioxopiperi- din-3-yl)-1-oxoisoindolin-5- yl)-4-hydroxypiperidin-1-yl)- methyl)phenyl)-N,N-dimeth- ylpiperidine-4-carboxamide |
| 42-A | 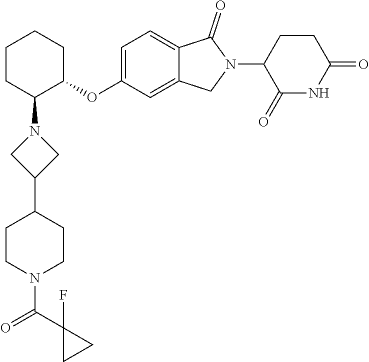 [SMILES: O=C1CCC(N2Cc3cc(O[C@H]4CCCC[C@@H]4N4CC(C5CCN(C(=O)C6(F)CC6)CC5)C4)ccc3C2=O)C(=O)N1] | 3-(5-(((1S,2S)-2-(3-(1-(1- fluorocyclopropane-1- carbonyl)piperidin-4-yl)- azetidin-1-yl)cyclohexyl)- oxy)-1-oxoisoindolin-2- yl)piperidine-2,6-dione |
| 43-A | 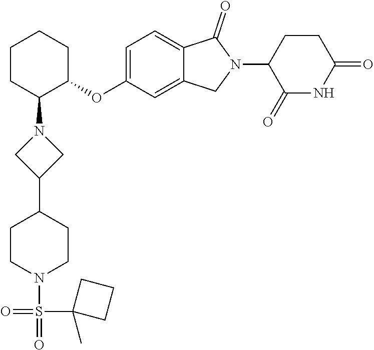 [SMILES: CC1(S(=O)(=O)N2CCC(C3CN([C@H]4CCCC[C@@H]4Oc4ccc5c(c4)CN(C4CCC(=O)NC4=O)C5=O)C3)CC2)CCC1] | 3-(5-(((1S,2S)-2-(3-(1-((1- methylcyclobutyl)sulfonyl)- piperidin-4-yl)azetidin-1- yl)cyclohexyl)oxy)-1-oxo- isoindolin-2-yl)piperidine- 2,6-dione |
| 44-A | 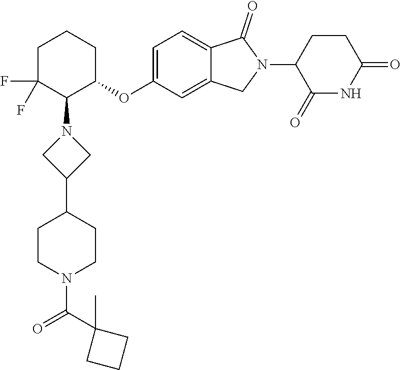 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCC1] | 3-(5-(((1S,2R)-3,3-difluoro- 2-(3-(1-(1-methylcyclo- butane-1-carbonyl)piperidin- 4-yl)azetidin-1-yl)cyclohex- yl)oxy)-1-oxoisoindolin-2- yl)piperidine-2,6-dione |
| 45-A | 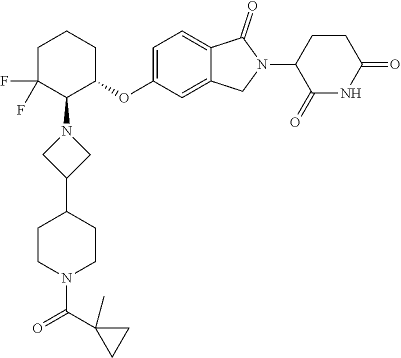 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CC1] | 3-(5-(((1S,2R)-3,3-difluoro- 2-(3-(1-(1-methylcyclopro- pane-1-carbonyl)piperidin-4- yl)azetidin-1-yl)cyclohexyl)- oxy)-1-oxoisoindolin-2-yl)- piperidine-2,6-dione |
| 46-A | 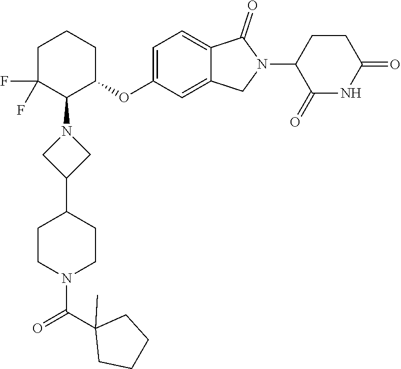 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCCC1] | 3-(5-(((1S.2R)-3,3-difluoro- 2-(3-(1-(1-methylcyclopen- tane-1-carbonyl)piperidin-4- yl)azetidin-1-yl)cyclohexyl)- oxy)-1-oxoisoindolin-2-yl)- piperidine-2,6-dione |
| 47-A | 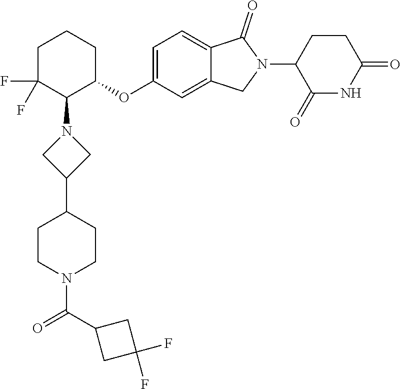 [SMILES: O=C1CCC(N2Cc3cc(O[C@H]4CCCC(F)(F)[C@@H]4N4CC(C5CCN(C(=O)C6CC(F)(F)C6)CC5)C4)ccc3C2=O)C(=O)N1] | 3-(5-(((1S,2R)-2-(3-(1-(3,3- difluorocyclobutane-1- carbonyl)piperidin-4-yl)- azetidin-1-yl)-3,3-difluoro- cyclohexyl)oxy)-1-oxoiso- indolin-2-yl)piperidine-2,6- dione |
| 48-A | 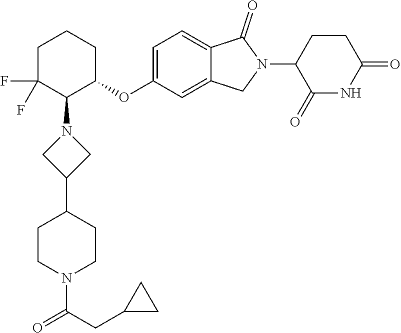 [SMILES: O=C1CCC(N2Cc3cc(O[C@H]4CCCC(F)(F)[C@@H]4N4CC(C5CCN(C(=O)CC6CC6)CC5)C4)ccc3C2=O)C(=O)N1] | 3-(5-(((1S,2R)-2-(3-(1-(2- cyclopropylacetyl)piperi- din-4-yl)azetidin-1-yl)-3,3- difluorocyclohexyl)oxy)- 1-oxoisoindolin-2-yl)- piperidine-2,6-dione |
| 49-A | 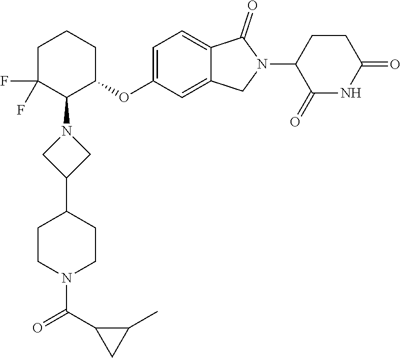 [SMILES: CC1CC1C(=O)N1CCC(C2CN([C@@H]3[C@@H](Oc4ccc5c(c4)CN(C4CCC(=O)NC4=O)C5=O)CCCC3(F)F)C2)CC1] | 3-(5-(((1S,2R)-3,3-difluoro- 2-(3-(1-(2-methylcyclopro- pane-1-carbonyl)piperidin- 4-yl)azetidin-1-yl)cyclohex- yl)oxy)-1-oxoisoindolin-2- yl)piperidine-2,6-dione |
| 50-A | 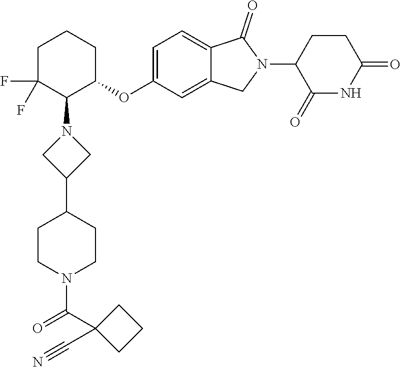 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CCC1] | 1-(4-(1-((1R,6S)-6-((2-(2,6- dioxopiperidin-3-yl)-1- oxoisoindolin-5-yl)oxy)- 2,2-difluorocyclohexyl)azeti- din-3-yl)piperidine-1-carbon- yl)cyclobutane-1-carbonitrile |
| 51-A | 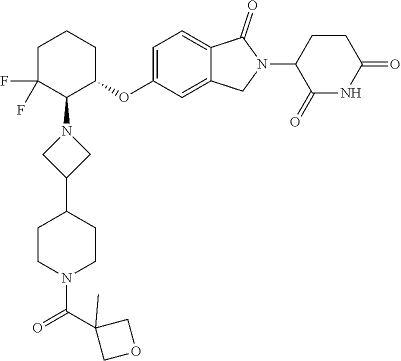 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)COC1] | 3-(5-(((1S,2R)-3,3-difluoro- 2-(3-(1-(3-methyloxetane- 3-carbonyl)piperidin-4-yl)- azetidin-1-yl)cyclohexyl)- oxy)-1-oxoisoindolin-2-yl)- piperidine-2,6-dione |
| 52-A | 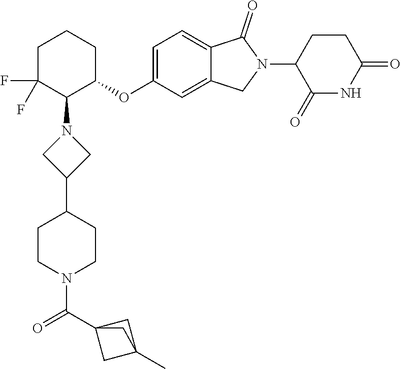 [SMILES: CC12CC(C(=O)N3CCC(C4CN([C@@H]5[C@@H](Oc6ccc7c(c6)CN(C6CCC(=O)NC6=O)C7=O)CCCC5(F)F)C4)CC3)(C1)C2] | 3-(5-(((1S,2R)-3,3-difluoro- 2-(3-(1-(3-methylbicyclo- [1.1.1]pentane-1-carbonyl)- piperidin-4-yl)azetidin-1- yl)cyclohexyl)oxy)-1-oxo- isoindolin-2-yl)piperidine- 2,6-dione |
| 53-A | 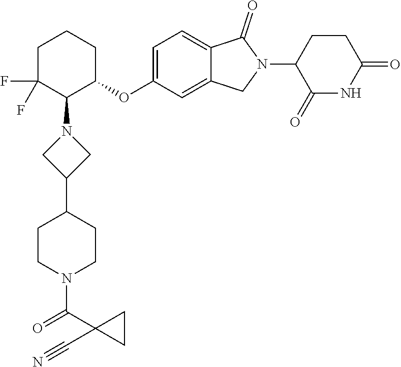 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CC1] | 1-(4-(1-((1R,6S)-6-((2-(2,6- dioxopiperidin-3-yl)-1-oxo- isoindolin-5-yl)oxy)-2,2- difluorocyclohexyl)azetidin- 3-yl)piperidine-1-carbonyl)- cyclopropane-1-carbonitrile |
| 54-A | 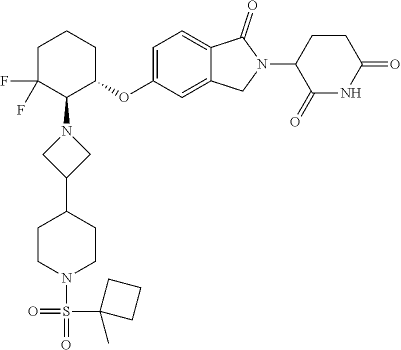 [SMILES: CC1(S(=O)(=O)N2CCC(C3CN([C@@H]4[C@@H](Oc5ccc6c(c5)CN(C5CCC(=O)NC5=O)C6=O)CCCC4(F)F)C3)CC2)CCC1] | 3-(5-(((1S.2R)-3,3-difluoro- 2-(3-(1-((1-methylcyclobut- yl)sulfonyl)piperidin-4-yl)- azetidin-1-yl)cyclohexyl)- oxy)-1-oxoisoindolin-2-yl)- piperidine-2,6-dione |
| 55-A | 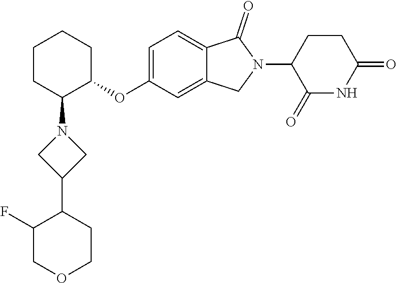 [SMILES: O=C1CCC(N2Cc3cc(O[C@H]4CCCC[C@@H]4N4CC(C5CCOCC5F)C4)ccc3C2=O)C(=O)N1] | 3-(5-(((1S,2S)-2-(3-(3- fluorotetrahydro-2H-pyran- 4-yl)azetidin-1-yl)cyclohex- yl)oxy)-1-oxoisoindolin-2- yl)piperidine-2,6-dione |
| 56-A | 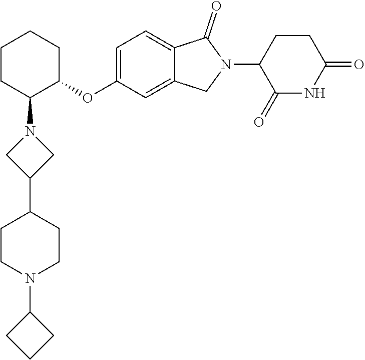 [SMILES: O=C1CCC(N2Cc3cc(O[C@H]4CCCC[C@@H]4N4CC(C5CCN(C6CCC6)CC5)C4)ccc3C2=O)C(=O)N1] | 3-(5-(((1S,2S)-2-(3-(1-cyclo- butylpiperidin-4-yl)-azetidin- 1-yl)cyclohexyl)oxy)-1-oxo- isoindolin-2-yl)piperidine- 2,6-dione |
| 57-A | 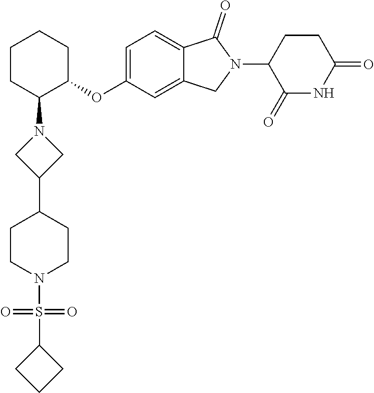 [SMILES: O=C1CCC(N2Cc3cc(O[C@H]4CCCC[C@@H]4N4CC(C5CCN(S(=O)(=O)C6CCC6)CC5)C4)ccc3C2=O)C(=O)N1] | 3-(5-(((1S,2S)-2-(3-(1- (cyclobutylsulfonyl)piperi- din-4-yl)azetidin-1-yl)cyclo- hexyl)oxy)-1-oxoisoindolin- 2-yl)piperidine-2,6-dione |
| 58-A | 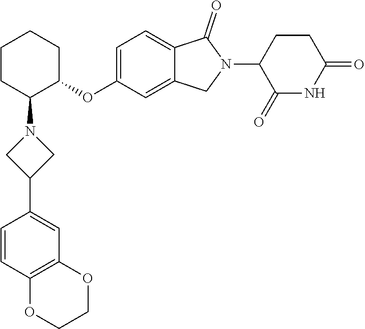 [SMILES: O=C1CCC(N2Cc3cc(O[C@H]4CCCC[C@@H]4N4CC(c5ccc6c(c5)OCCO6)C4)ccc3C2=O)C(=O)N1] | 3-(5-(((1S,2S)-2-(3-(2,3- dihydrobenzo[b][1,4]dioxin- 6-yl)azetidin-1-yl)cyclohex- yl)oxy)-1-oxoisoindolin-2- yl)piperidine-2,6-dione |
| 1-B | 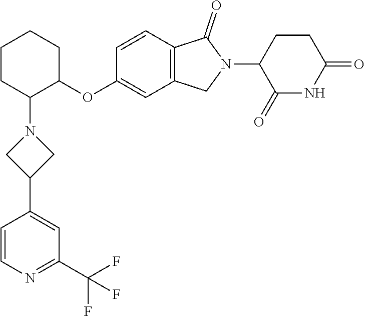 [SMILES: O=C1CCC(N2Cc3cc(OC4CCCCC4N4CC(c5ccnc(C(F)(F)F)c5)C4)ccc3C2=O)C(=O)N1] | 3-(1-oxo-5-((2-(3-(2- (trifluoromethyl)-pyridin-4- yl)azetidin-1-yl)cyclohexyl)- oxy)isoindolin-2-yl)piperi- dine-2,6-dione |
| 2-B | 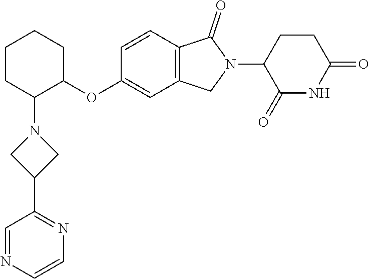 [SMILES: O=C1CCC(N2Cc3cc(OC4CCCCC4N4CC(c5cnccn5)C4)ccc3C2=O)C(=O)N1] | 3-(1-oxo-5-((2-(3-(pyrazin- 2-yl)azetidin-1-yl)cyclohex- yl)oxy)isoindolin-2-yl)- piperidine-2,6-dione |
| 3-B | 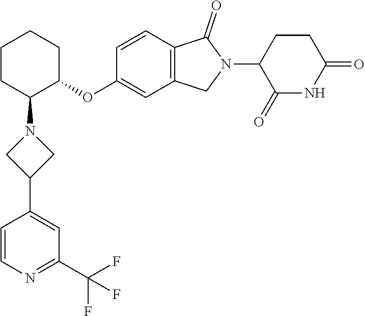 [SMILES: O=C1CCC(N2Cc3cc(O[C@H]4CCCC[C@@H]4N4CC(c5ccnc(C(F)(F)F)c5)C4)ccc3C2=O)C(=O)N1] | 3-(1-oxo-5-(((1S,2S)-2-(3- (2-(trifluoromethyl)pyridin- 4-yl)azetidin-1-yl)cyclohex- yl)oxy)isoindolin-2-yl)piperi- dine-2,6-dione |
| 4-B | 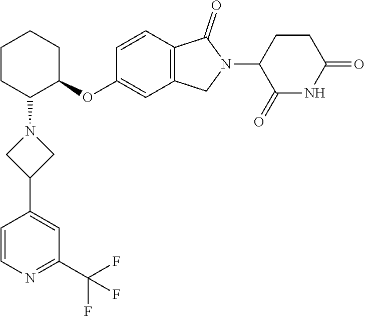 | 3-(1-oxo-5-(((1R,2R)-2-(3- (2-(trifluoromethyl)pyridin- 4-yl)azetidin-1-yl)cyclohex- yl)oxy)isoindolin-2-yl)piperi- dine-2,6-dione |
| 5-B | 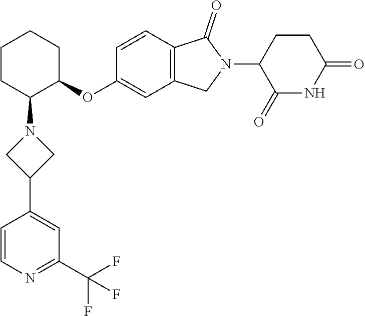 [SMILES: O=C1CCC(N2Cc3cc(O[C@@H]4CCCC[C@@H]4N4CC(c5ccnc(C(F)(F)F)c5)C4)ccc3C2=O)C(=O)N1] | 3-(1-oxo-5-(((1R,2S)-2-(3- (2-(trifluoromethyl)pyridin- 4-yl)azetidin-1-yl)cyclohex- yl)oxy)isoindolin-2-yl)piperi- dine-2,6-dione |
| 6-B | 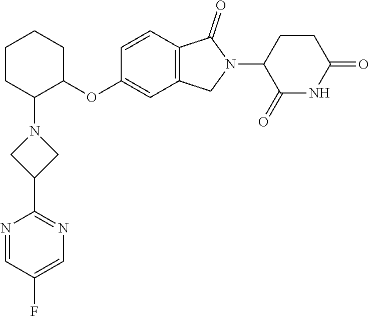 [SMILES: O=C1CCC(N2Cc3cc(OC4CCCCC4N4CC(c5ncc(F)cn5)C4)ccc3C2=O)C(=O)N1] | 3-(5-((2-(3-(5-fluoropyrimi- din-2-yl)azetidin-1-yl)cyclo- hexyl)-oxy)-1-oxoisoindolin- 2-yl)piperidine-2,6-dione |
| 7-B | 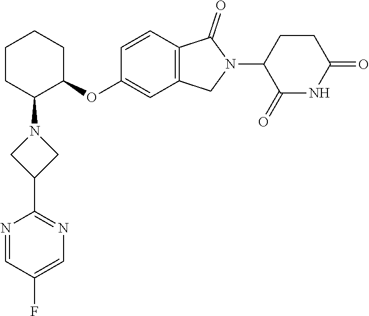 [SMILES: O=C1CCC(N2Cc3cc(O[C@@H]4CCCC[C@@H]4N4CC(c5ncc(F)cn5)C4)ccc3C2=O)C(=O)N1] | 3-(5-(((1R,2S)-2-(3-(5- fluoropyrimidin-2-yl)azeti- din-1-yl)cyclohexyl)oxy)-1- oxoisoindolin-2-yl)piperi- dine-2,6-dione |
| 8-B | 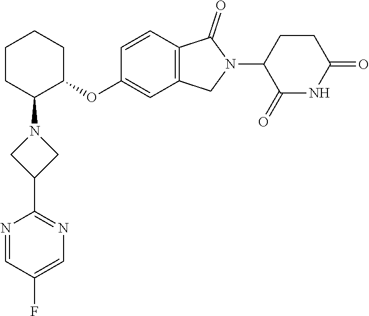 [SMILES: O=C1CCC(N2Cc3cc(O[C@H]4CCCC[C@@H]4N4CC(c5ncc(F)cn5)C4)ccc3C2=O)C(=O)N1] | 3-(5-(((1S,2S)-2-(3-(5- fluoropyrimidin-2-yl)azeti- din-1-yl)cyclohexyl)oxy)-1- oxoisoindolin-2-yl)piperi- dine-2,6-dione |
| 9-B | 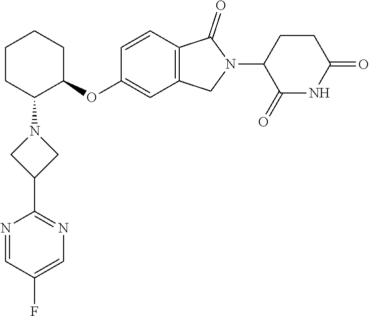 [SMILES: O=C1CCC(N2Cc3cc(O[C@@H]4CCCC[C@H]4N4CC(c5ncc(F)cn5)C4)ccc3C2=O)C(=O)N1] | 3-(5-(((1R,2R)-2-(3-(5- fluoropyrimidin-2-yl)azeti- din-1-yl)cyclohexyl)oxy)-1- oxoisoindolin-2-yl)piperi- dine-2,6-dione |
| 10-B | 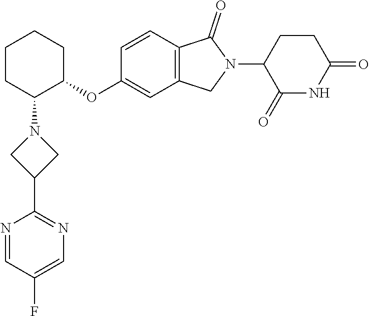 [SMILES: O=C1CCC(N2Cc3cc(O[C@H]4CCCC[C@H]4N4CC(c5ncc(F)cn5)C4)ccc3C2=O)C(=O)N1] | 3-(5-(((1S,2R)-2-(3-(5-fluoro- pyrimidin-2-yl)azetidin-1- yl)cyclohexyl)oxy)-1-oxoiso- indolin-2-yl)piperidine-2,6- dione |
| 11-B | 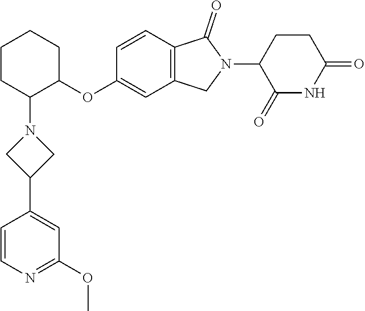 [SMILES: COc1cc(C2CN(C3CCCCC3Oc3ccc4c(c3)CN(C3CCC(=O)NC3=O)C4=O)C2)ccn1] | 3-(5-((2-(3-(2-methoxypyri- din-4-yl)azetidin-1-yl)cyclo- hexyl)oxy)-1-oxoisoindolin- 2-yl)piperidine-2,6-dione |
| 12-B | 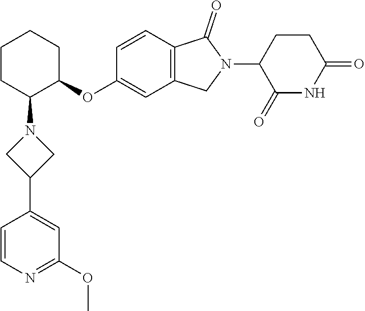 [SMILES: COc1cc(C2CN([C@H]3CCCC[C@H]3Oc3ccc4c(c3)CN(C3CCC(=O)NC3=O)C4=O)C2)ccn1] | 3-(5-(((1R,2S)-2-(3-(2-meth- oxypyridin-4-yl)azetidin-1- yl)cyclohexyl)oxy)-1-oxoiso- indolin-2-yl)piperidine-2,6- dione |
| 13-B | 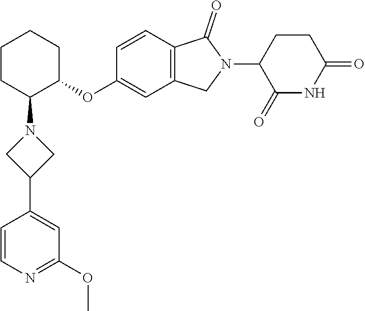 [SMILES: COc1cc(C2CN([C@H]3CCCC[C@@H]3Oc3ccc4c(c3)CN(C3CCC(=O)NC3=O)C4=O)C2)ccn1] | 3-(5-(((1S,2S)-2-(3-(2-meth- oxypyridin-4-yl)azetidin-1- yl)cyclohexyl)oxy)-1-oxoiso- indolin-2-yl)piperidine-2,6- dione |
| 14-B | 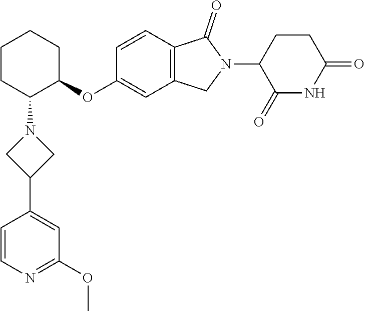 [SMILES: COc1cc(C2CN([C@@H]3CCCC[C@H]3Oc3ccc4c(c3)CN(C3CCC(=O)NC3=O)C4=O)C2)ccn1] | 3-(5-(((1R,2R)-2-(3-(2-meth- oxypyridin-4-yl)azetidin-1- yl)cyclohexyl)oxy)-1-oxoiso- indolin-2-yl)piperidine-2,6- dione |
| 15-B | 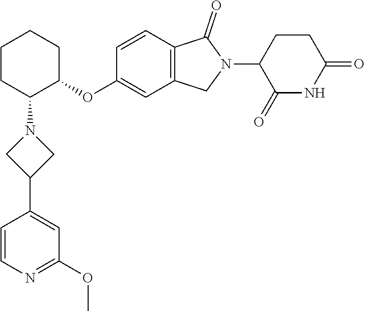 [SMILES: COc1cc(C2CN([C@@H]3CCCC[C@@H]3Oc3ccc4c(c3)CN(C3CCC(=O)NC3=O)C4=O)C2)ccn1] | 3-(5-(((1S,2R)-2-(3-(2-meth- oxypyridin-4-yl)azetidin-1- yl)cyclohexyl)oxy)-1-oxoiso- indolin-2-yl)piperidine-2,6- dione |
| 16-B | 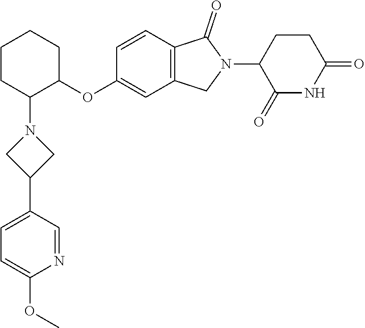 [SMILES: COc1ccc(C2CN(C3CCCCC3Oc3ccc4c(c3)CN(C3CCC(=O)NC3=O)C4=O)C2)cn1] | 3-(5-((2-(3-(6-methoxypyri- din-3-yl)azetidin-1-yl)cyclo- hexyl)oxy)-1-oxoisoindolin- 2-yl)piperidine-2,6-dione |
| 17-B | 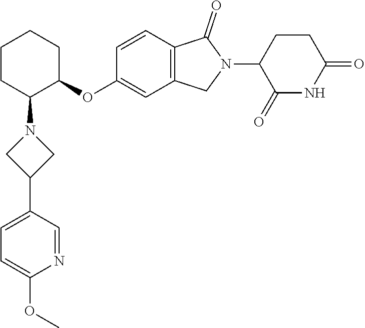 [SMILES: COc1ccc(C2CN([C@H]3CCCC[C@H]3Oc3ccc4c(c3)CN(C3CCC(=O)NC3=O)C4=O)C2)cn1] | 3-(5-(((1R,2S)-2-(3-(6-meth- oxypyridin-3-yl)azetidin-1- yl)cyclohexyl)oxy)-1-oxoiso- indolin-2-yl)piperidine-2,6- dione |
| 18-B | 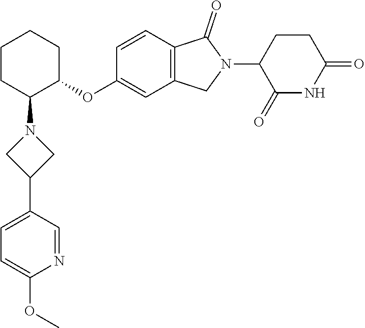 [SMILES: COc1ccc(C2CN([C@H]3CCCC[C@@H]3Oc3ccc4c(c3)CN(C3CCC(=O)NC3=O)C4=O)C2)cn1] | 3-(5-(((1S,2S)-2-(3-(6-meth- oxypyridin-3-yl)azetidin-1- yl)cyclohexyl)oxy)-1-oxoiso- indolin-2-yl)piperidine-2,6- dione |
| 19-B | 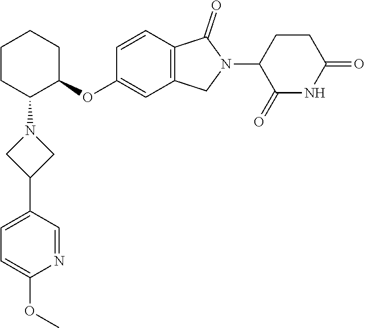 [SMILES: COc1ccc(C2CN([C@@H]3CCCC[C@H]3Oc3ccc4c(c3)CN(C3CCC(=O)NC3=O)C4=O)C2)cn1] | 3-(5-(((1R,2R)-2-(3-(6-meth- oxypyridin-3-yl)azetidin-1- yl)cyclohexyl)oxy)-1-oxoiso- indolin-2-yl)piperidine-2,6- dione |
| 20-B | 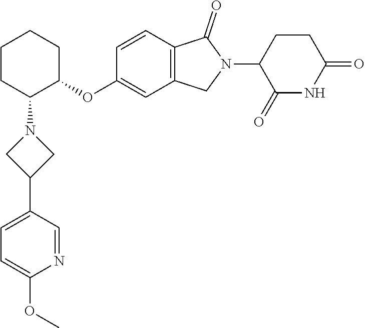 [SMILES: COc1ccc(C2CN([C@@H]3CCCC[C@@H]3Oc3ccc4c(c3)CN(C3CCC(=O)NC3=O)C4=O)C2)cn1] | 3-(5-(((1S,2R)-2-(3-(6-meth- oxypyridin-3-yl)azetidin-1- yl)cyclohexyl)oxy)-1-oxoiso- indolin-2-yl)piperidine-2,6- dione |
| 21-B | 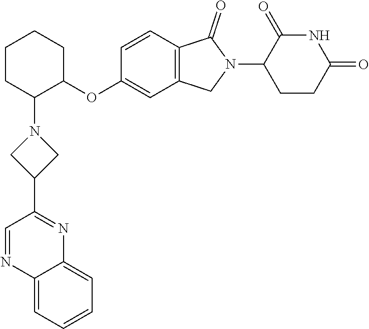 [SMILES: O=C1CCC(N2Cc3cc(OC4CCCCC4N4CC(c5cnc6ccccc6n5)C4)ccc3C2=O)C(=O)N1] | 3-(1-oxo-5-((2-(3-(quinox- alin-2-yl)azetidin-1-yl)cyclo- hexyl)oxy)isoindolin-2-yl)- piperidine-2,6-dione |
| 22-B | 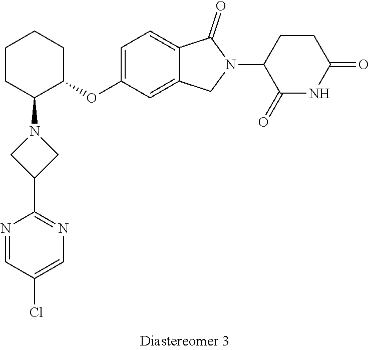 [SMILES: O=C1CCC(N2Cc3cc(O[C@H]4CCCC[C@@H]4N4CC(c5ncc(Cl)cn5)C4)ccc3C2=O)C(=O)N1] | 3-(1-oxo-5-(((1S,2R)-2-(3- (quinoxalin-2-yl)azetidin-1- yl)cyclohexyl)oxy)isoindolin- 2-yl)piperidine-2,6-dione |
| 23-B | 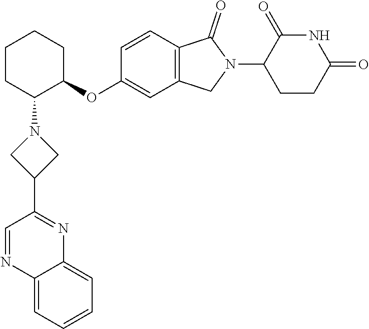 [SMILES: O=C1CCC(N2Cc3cc(O[C@@H]4CCCC[C@H]4N4CC(c5cnc6ccccc6n5)C4)ccc3C2=O)C(=O)N1] | 3-(1-oxo-5-(((1R,2R)-2-(3- (quinoxalin-2-yl)azetidin-1- yl)cyclohexyl)oxy)isoindolin- 2-yl)piperidine-2,6-dione |
| 24-B | 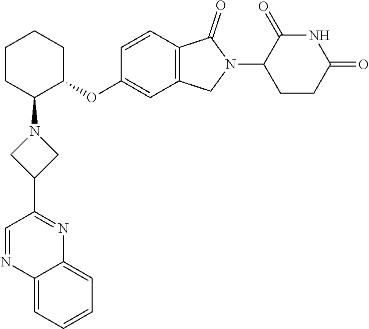 [SMILES: O=C1CCC(N2Cc3cc(O[C@H]4CCCC[C@@H]4N4CC(c5cnc6ccccc6n5)C4)ccc3C2=O)C(=O)N1] | 3-(1-oxo-5-(((1S,2S)-2-(3- (quinoxalin-2-yl)azetidin-1- yl)cyclohexyl)oxy)isoindolin- 2-yl)piperidine-2,6-dione |
| 25-B | 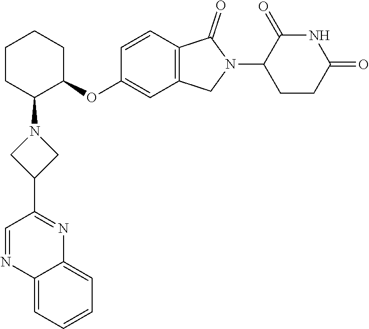 [SMILES: O=C1CCC(N2Cc3cc(O[C@@H]4CCCC[C@@H]4N4CC(c5cnc6ccccc6n5)C4)ccc3C2=O)C(=O)N1] | 3-(1-oxo-5-(((1R,2S)-2-(3- (quinoxalin-2-yl)azetidin-1- yl)cyclohexyl)oxy)isoindolin- 2-yl)piperidine-2,6-dione |
| 26-B | 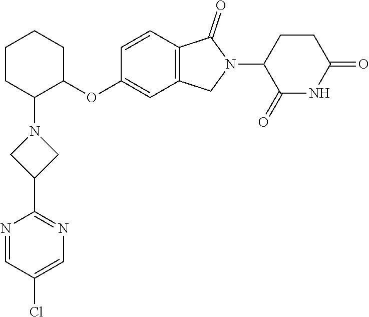 [SMILES: O=C1CCC(N2Cc3cc(OC4CCCCC4N4CC(c5ncc(Cl)cn5)C4)ccc3C2=O)C(=O)N1] | 3-(5-((2-(3-(5-chloropyrimi- din-2-yl)azetidin-1-yl)cyclo- hexyl)oxy)-1-oxoisoindolin- 2-yl)piperidine-2,6-dione |
| 27-B | 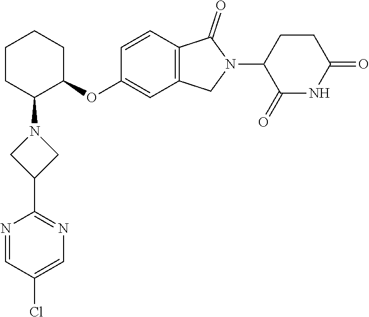 [SMILES: O=C1CCC(N2Cc3cc(O[C@@H]4CCCC[C@@H]4N4CC(c5ncc(Cl)cn5)C4)ccc3C2=O)C(=O)N1] | 3-(5-(((1R,2S)-2-(3-(5-chloro- pyrimidin-2-yl)azetidin-1- yl)cyclohexyl)oxy)-1-oxoiso- indolin-2-yl)piperidine-2,6- dione |
| 28-B | 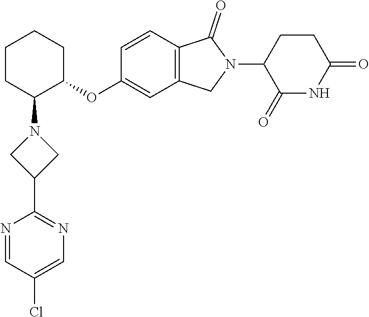 [SMILES: O=C1CCC(N2Cc3cc(O[C@H]4CCCC[C@@H]4N4CC(c5ncc(Cl)cn5)C4)ccc3C2=O)C(=O)N1] | 3-(5-(((1S,2S)-2-(3-(5-chloro- pyrimidin-2-yl)azetidin-1-yl)- cyclohexyl)oxy)-1-oxoiso- indolin-2-yl)piperidine-2,6- dione |
| 29-B | 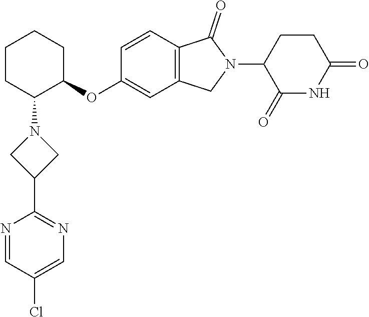 [SMILES: O=C1CCC(N2Cc3cc(O[C@@H]4CCCC[C@H]4N4CC(c5ncc(Cl)cn5)C4)ccc3C2=O)C(=O)N1] | 3-(5-(((1R,2R)-2-(3-(5-chloro- pyrimidin-2-yl)azetidin-1- yl)cyclohexyl)oxy)-1-oxoiso- indolin-2-yl)piperidine-2,6- dione |
| 30-B | 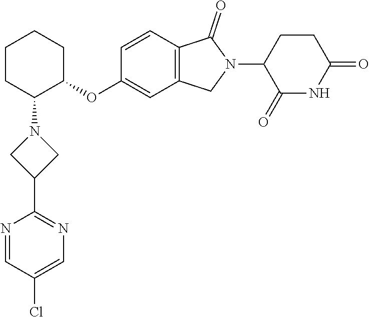 [SMILES: O=C1CCC(N2Cc3cc(O[C@H]4CCCC[C@H]4N4CC(c5ncc(Cl)cn5)C4)ccc3C2=O)C(=O)N1] | 3-(5-(((1S,2R)-2-(3-(5-chloro- pyrimidin-2-yl)azetidin-1-yl)- cyclohexyl)oxy)-1-oxoiso- indolin-2-yl)piperidine-2,6- dione |
| 31-B | 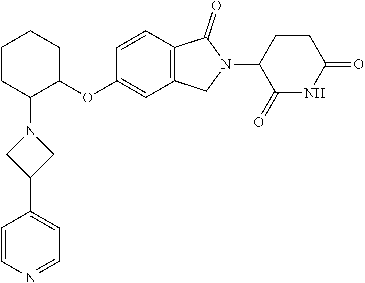 [SMILES: O=C1CCC(N2Cc3cc(OC4CCCCC4N4CC(c5ccncc5)C4)ccc3C2=O)C(=O)N1] | 3-(1-oxo-5-((2-(3-(pyridin-4- yl)azetidin-1-yl)cyclohexyl)- oxy)isoindolin-2-yl)piperi- dine-2,6-dione |
| 32-B | 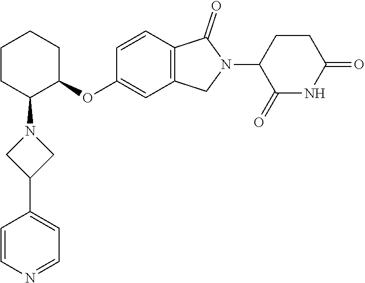 [SMILES: O=C1CCC(N2Cc3cc(O[C@@H]4CCCC[C@@H]4N4CC(c5ccncc5)C4)ccc3C2=O)C(=O)N1] | 3-(1-oxo-5-(((1R,2S)-2-(3- (pyridin-4-yl)azetidin-1-yl)- cyclohexyl)oxy)isoindolin-2- yl)piperidine-2,6-dione |
| 33-B | 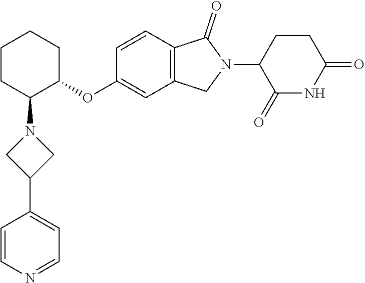 [SMILES: O=C1CCC(N2Cc3cc(O[C@H]4CCCC[C@@H]4N4CC(c5ccncc5)C4)ccc3C2=O)C(=O)N1] | 3-(1-oxo-5-(((1S,2S)-2-(3- (pyridin-4-yl)azetidin-1-yl)- cyclohexyl)oxy)isoindolin-2- yl)piperidine-2,6-dione |
| 34-B | 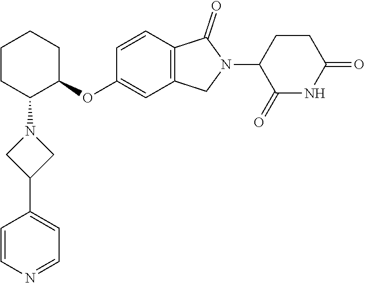 [SMILES: O=C1CCC(N2Cc3cc(O[C@@H]4CCCC[C@H]4N4CC(c5ccncc5)C4)ccc3C2=O)C(=O)N1] | 3-(1-oxo-5-(((1R,2R)-2-(3- (pyridin-4-yl)azetidin-1-yl)- cyclohexyl)oxy)isoindolin-2- yl)piperidine-2,6-dione |
| 35-B | 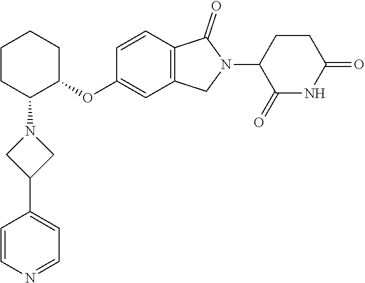 [SMILES: O=C1CCC(N2Cc3cc(O[C@H]4CCCC[C@H]4N4CC(c5ccncc5)C4)ccc3C2=O)C(=O)N1] | 3-(1-oxo-5-(((1S,2R)-2-(3- (pyridin-4-yl)azetidin-1-yl)- cyclohexyl)oxy)isoindolin-2- yl)piperidine-2,6-dione |
| 36-B | 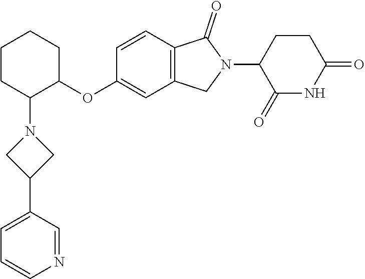 [SMILES: O=C1CCC(N2Cc3cc(OC4CCCCC4N4CC(c5cccnc5)C4)ccc3C2=O)C(=O)N1] | 3-(1-oxo-5-((2-(3-(pyridin-3- yl)azetidin-1-yl)cyclohexyl)- oxy)isoindolin-2-yl)piperi- dine-2,6-dione |
| 37-B | 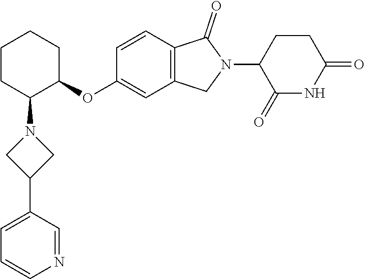 [SMILES: O=C1CCC(N2Cc3cc(O[C@@H]4CCCC[C@@H]4N4CC(c5cccnc5)C4)ccc3C2=O)C(=O)N1] | 3-(1-oxo-5-(((1R,2S)-2-(3- (pyridin-3-yl)azetidin-1-yl)- cyclohexyl)oxy)isoindolin-2- yl)piperidine-2,6-dione |
| 38-B | 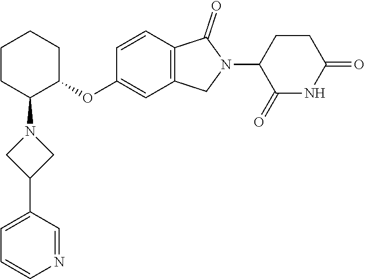 [SMILES: O=C1CCC(N2Cc3cc(O[C@H]4CCCC[C@@H]4N4CC(c5cccnc5)C4)ccc3C2=O)C(=O)N1] | 3-(1-oxo-5-(((1S,2S)-2-(3- (pyridin-3-yl)azetidin-1-yl)- cyclohexyl)oxy)isoindolin-2- yl)piperidine-2,6-dione |
| 39-B | 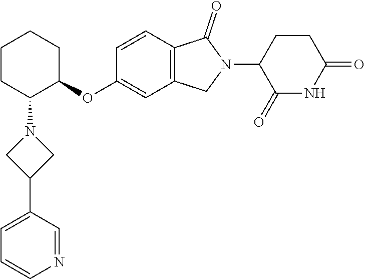 [SMILES: O=C1CCC(N2Cc3cc(O[C@@H]4CCCC[C@H]4N4CC(c5cccnc5)C4)ccc3C2=O)C(=O)N1] | 3-(1-oxo-5-(((1R,2R)-2-(3- (pyridin-3-yl)azetidin-1-yl)- cyclohexyl)oxy)isoindolin-2- yl)piperidine-2,6-dione |
| 40-B | 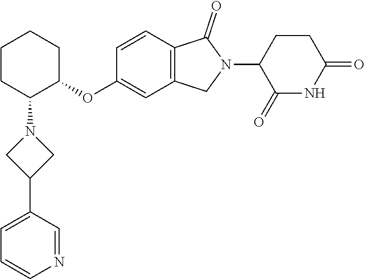 [SMILES: O=C1CCC(N2Cc3cc(O[C@H]4CCCC[C@H]4N4CC(c5cccnc5)C4)ccc3C2=O)C(=O)N1] | 3-(1-oxo-5-(((1S,2R)-2-(3- (pyridin-3-yl)azetidin-1-yl)- cyclohexyl)oxy)isoindolin-2- yl)piperidine-2,6-dione |
| 41-B | 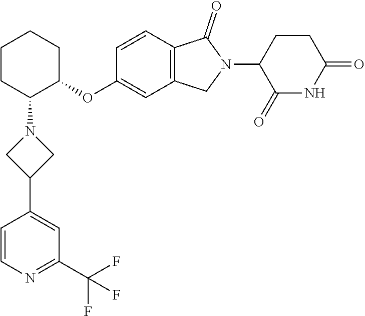 [SMILES: O=C1CCC(N2Cc3cc(O[C@H]4CCCC[C@H]4N4CC(c5ccnc(C(F)(F)F)c5)C4)ccc3C2=O)C(=O)N1] | 3-(1-oxo-5-(((1S,2R)-2-(3- (2-(trifluoromethyl)pyridin- 4-yl)azetidin-1-yl)cyclohex- yl)oxy)isoindolin-2-yl)piperi- dine-2,6-dione |
| 42-B | 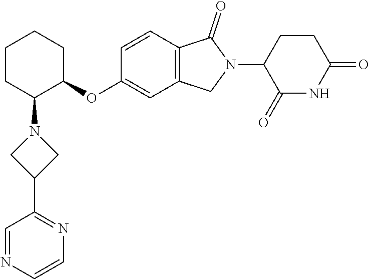 [SMILES: O=C1CCC(N2Cc3cc(O[C@@H]4CCCC[C@@H]4N4CC(c5cnccn5)C4)ccc3C2=O)C(=O)N1] | 3-(1-oxo-5-(((1R,2S)-2-(3- (pyrazin-2-yl)azetidin-1- yl)cyclohexyl)oxy)isoindolin- 2-yl)piperidine-2,6-dione |
| 43-B | 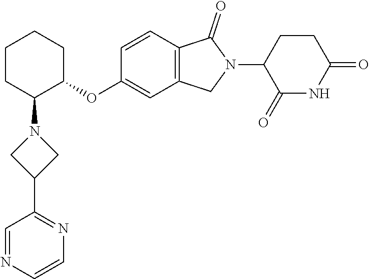 [SMILES: O=C1CCC(N2Cc3cc(O[C@H]4CCCC[C@@H]4N4CC(c5cnccn5)C4)ccc3C2=O)C(=O)N1] | 3-(1-oxo-5-(((1S,2S)-2-(3- (pyrazin-2-yl)azetidin-1-yl)- cyclohexyl)oxy)isoindolin-2- yl)piperidine-2,6-dione |
| 44-B | 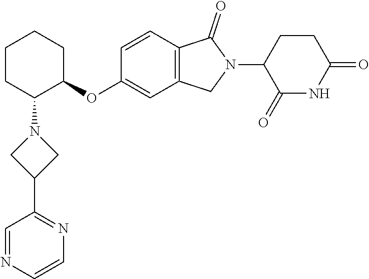 [SMILES: O=C1CCC(N2Cc3cc(O[C@@H]4CCCC[C@H]4N4CC(c5cnccn5)C4)ccc3C2=O)C(=O)N1] | 3-(1-oxo-5-(((1R,2R)-2-(3- (pyrazin-2-yl)azetidin-1-yl)- cyclohexyl)oxy)isoindolin-2- yl)piperidine-2,6-dione |
| 45-B | 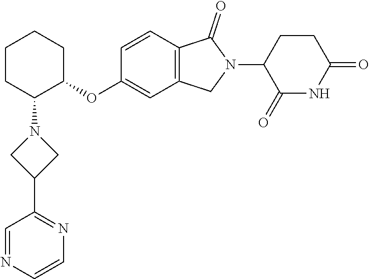 [SMILES: O=C1CCC(N2Cc3cc(O[C@H]4CCCC[C@H]4N4CC(c5cnccn5)C4)ccc3C2=O)C(=O)N1] | 3-(1-oxo-5-(((1S,2R)-2-(3- (pyrazin-2-yl)azetidin-1-yl)- cyclohexyl)oxy)isoindolin-2- yl)piperidine-2,6-dione |
| 46-B | 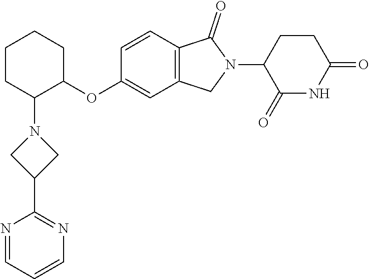 [SMILES: O=C1CCC(N2Cc3cc(OC4CCCCC4N4CC(c5ncccn5)C4)ccc3C2=O)C(=O)N1] | 3-(1-oxo-5-((2-(3-(pyrimidin- 2-yl)azetidin-1-yl)cyclohex- yl)oxy)-isoindolin-2-yl)- piperidine-2,6-dione |
| 47-B | 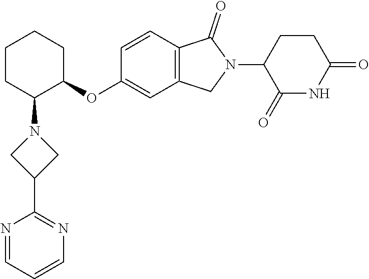 [SMILES: O=C1CCC(N2Cc3cc(O[C@@H]4CCCC[C@@H]4N4CC(c5ncccn5)C4)ccc3C2=O)C(=O)N1] | 3-(1-oxo-5-(((1R,2S)-2-(3- (pyrimidin-2-yl)azetidin-1- yl)cyclohexyl)oxy)isoindolin- 2-yl)piperidine-2,6-dione |
| 48-B | 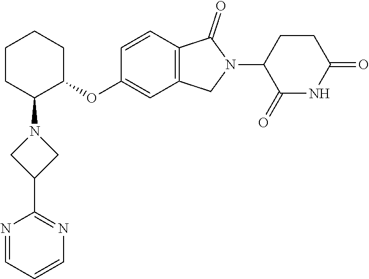 [SMILES: O=C1CCC(N2Cc3cc(O[C@H]4CCCC[C@@H]4N4CC(c5ncccn5)C4)ccc3C2=O)C(=O)N1] | 3-(1-oxo-5-(((1S,2S)-2-(3- (pyrimidin-2-yl)azetidin-1- yl)cyclohexyl)oxy)isoindolin- 2-yl)piperidine-2,6-dione |
| 49-B | 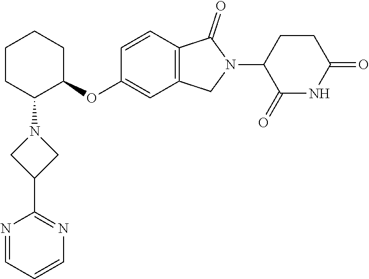 [SMILES: O=C1CCC(N2Cc3cc(O[C@@H]4CCCC[C@H]4N4CC(c5ncccn5)C4)ccc3C2=O)C(=O)N1] | 3-(1-oxo-5-(((1R,2R)-2-(3- (pyrimidin-2-yl)azetidin-1- yl)cyclohexyl)oxy)isoindolin- 2-yl)piperidine-2,6-dione |
| 50-B | 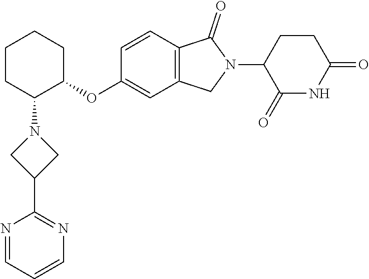 [SMILES: O=C1CCC(N2Cc3cc(O[C@H]4CCCC[C@H]4N4CC(c5ncccn5)C4)ccc3C2=O)C(=O)N1] | 3-(1-oxo-5-(((1S,2R)-2-(3- (pyrimidin-2-yl)azetidin-1- yl)cyclohexyl)oxy)isoindolin- 2-yl)piperidine-2,6-dione |
| 51-B | 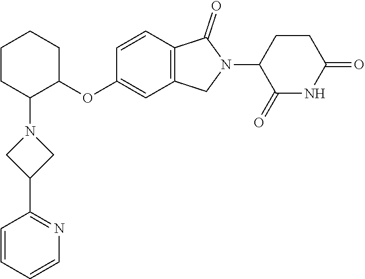 [SMILES: O=C1CCC(N2Cc3cc(OC4CCCCC4N4CC(c5ccccn5)C4)ccc3C2=O)C(=O)N1] | 3-(1-oxo-5-((2-(3-(pyridin-2- yl)azetidin-1-yl)cyclohexyl)- oxy)isoindolin-2-yl)piperi- dine-2,6-dione |
| 52-B | 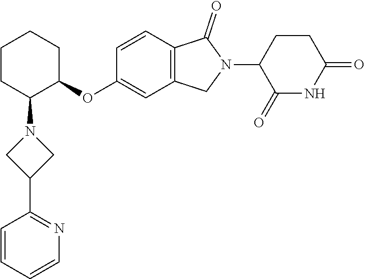 [SMILES: O=C1CCC(N2Cc3cc(O[C@@H]4CCCC[C@@H]4N4CC(c5ccccn5)C4)ccc3C2=O)C(=O)N1] | 3-(1-oxo-5-(((1R,2S)-2-(3- (pyridin-2-yl)azetidin-1-yl)- cyclohexyl)oxy)isoindolin-2- yl)piperidine-2,6-dione |
| 53-B | 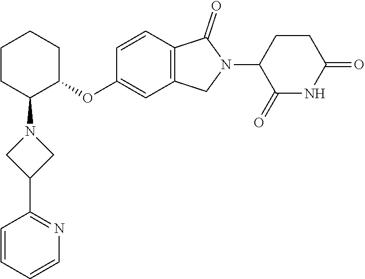 [SMILES: O=C1CCC(N2Cc3cc(O[C@H]4CCCC[C@@H]4N4CC(c5ccccn5)C4)ccc3C2=O)C(=O)N1] | 3-(1-oxo-5-(((1S,2S)-2-(3- (pyridin-2-yl)azetidin-1-yl)- cyclohexyl)oxy)isoindolin-2- yl)piperidine-2,6-dione |
| 54-B | 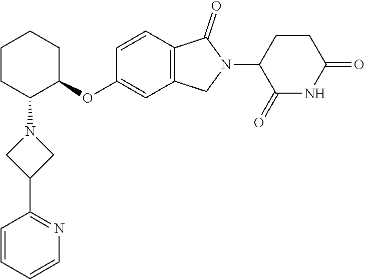 [SMILES: O=C1CCC(N2Cc3cc(O[C@@H]4CCCC[C@H]4N4CC(c5ccccn5)C4)ccc3C2=O)C(=O)N1] | 3-(1-oxo-5-(((1R,2R)-2-(3- (pyridin-2-yl)azetidin-1-yl)- cyclohexyl)oxy)isoindolin-2- yl)piperidine-2,6-dione |
| 55-B | 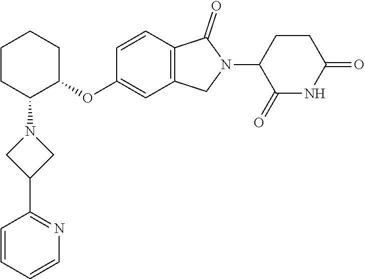 [SMILES: O=C1CCC(N2Cc3cc(O[C@H]4CCCC[C@H]4N4CC(c5ccccn5)C4)ccc3C2=O)C(=O)N1] | 3-(1-oxo-5-(((1S,2R)-2-(3- (pyridin-2-yl)azetidin-1-yl)- cyclohexyl)oxy)isoindolin-2- yl)piperidine-2,6-dione |
| 56-B | 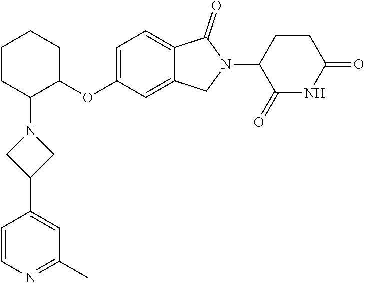 [SMILES: Cc1cc(C2CN(C3CCCCC3Oc3ccc4c(c3)CN(C3CCC(=O)NC3=O)C4=O)C2)ccn1] | 3-(5-((2-(3-(2-methylpyridin- 4-yl)azetidin-1-yl)cyclohex- yl)oxy)-1-oxoisoindolin-2- yl)piperidine-2,6-dione |
| 57-B | 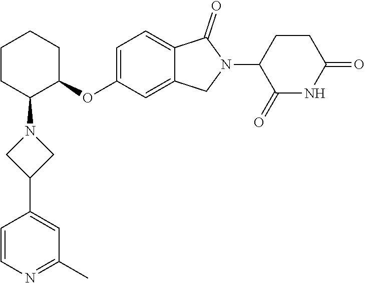 [SMILES: Cc1cc(C2CN([C@H]3CCCC[C@H]3Oc3ccc4c(c3)CN(C3CCC(=O)NC3=O)C4=O)C2)ccn1] | 3-(5-(((1R,2S)-2-(3-(2-meth- ylpyridin-4-yl)azetidin-1-yl)- cyclohexyl)oxy)-1-oxoiso- indolin-2-yl)piperidine-2,6- dione |
| 58-B | 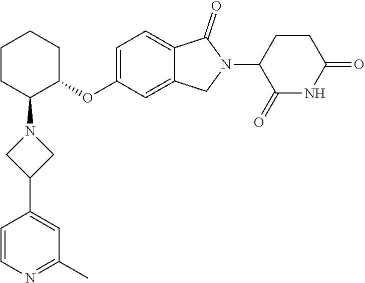 [SMILES: Cc1cc(C2CN([C@H]3CCCC[C@@H]3Oc3ccc4c(c3)CN(C3CCC(=O)NC3=O)C4=O)C2)ccn1] | 3-(5-(((1S,2S)-2-(3-(2-meth- ylpyridin-4-yl)azetidin-1-yl)- cyclohexyl)oxy)-1-oxoiso- indolin-2-yl)piperidine-2,6- dione |
| 59-B | 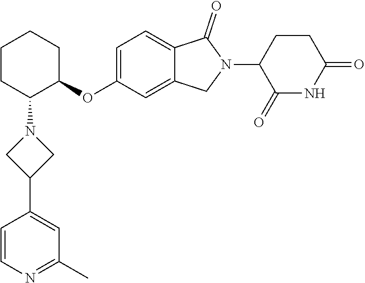 [SMILES: Cc1cc(C2CN([C@@H]3CCCC[C@H]3Oc3ccc4c(c3)CN(C3CCC(=O)NC3=O)C4=O)C2)ccn1] | 3-(5-(((1R,2R)-2-(3-(2-meth- ylpyridin-4-yl)azetidin-1- yl)cyclohexyl)oxy)-1-oxoiso- indolin-2-yl)piperidine-2,6- dione |
| 60-B | 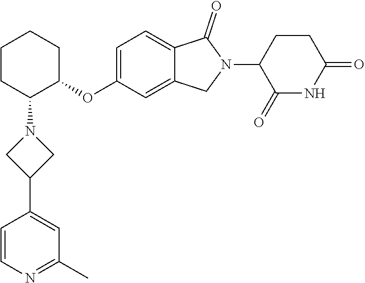 [SMILES: Cc1cc(C2CN([C@@H]3CCCC[C@@H]3Oc3ccc4c(c3)CN(C3CCC(=O)NC3=O)C4=O)C2)ccn1] | 3-(5-(((1S,2R)-2-(3-(2-meth- ylpyridin-4-yl)azetidin-1-yl)- cyclohexyl)oxy)-1-oxoiso- indolin-2-yl)piperidine-2,6- dione |
| 61-B | 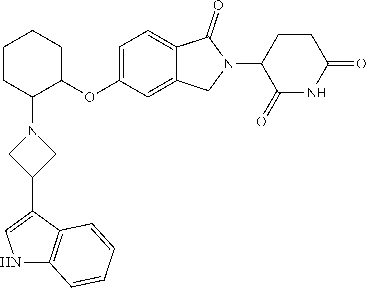 [SMILES: O=C1CCC(N2Cc3cc(OC4CCCCC4N4CC(c5c[nH]c6ccccc56)C4)ccc3C2=O)C(=O)N1] | 3-(5-((2-(3-(1H-indol-3-yl)- azetidin-1-yl)cyclohexyl)- oxy)-1-oxoisoindolin-2-yl)- piperidine-2,6-dione |
| 62-B | 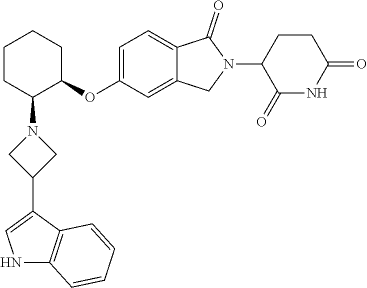 [SMILES: O=C1CCC(N2Cc3cc(O[C@@H]4CCCC[C@@H]4N4CC(c5c[nH]c6ccccc56)C4)ccc3C2=O)C(=O)N1] | 3-(5-(((1R,2S)-2-(3-(1H- indol-3-yl)azetidin-1-yl)cyclo- hexyl)oxy)-1-oxoisoindolin-2- yl)piperidine-2,6-dione |
| 63-B | 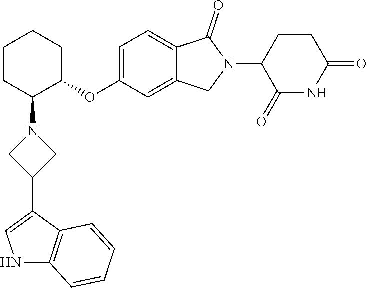 [SMILES: O=C1CCC(N2Cc3cc(O[C@H]4CCCC[C@@H]4N4CC(c5c[nH]c6ccccc56)C4)ccc3C2=O)C(=O)N1] | 3-(5-(((1S,2S)-2-(3-(1H- indol-3-yl)azetidin-1-yl)- cyclohexyl)oxy)-1-oxoiso- indolin-2-yl)piperidine-2,6- dione |
| 64-B | 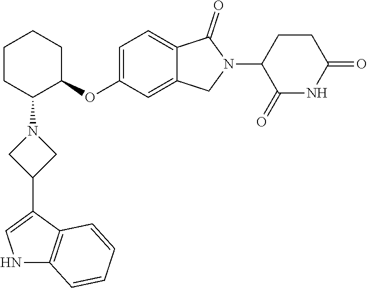 [SMILES: O=C1CCC(N2Cc3cc(O[C@@H]4CCCC[C@H]4N4CC(c5c[nH]c6ccccc56)C4)ccc3C2=O)C(=O)N1] | 3-(5-(((1R,2R)-2-(3-(1H- indol-3-yl)azetidin-1-yl)- cyclohexyl)oxy)-1-oxoiso- indolin-2-yl)piperidine-2,6- dione |
| 65-B | 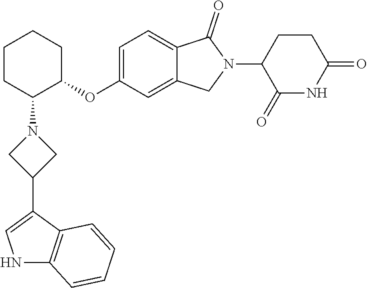 [SMILES: O=C1CCC(N2Cc3cc(O[C@H]4CCCC[C@H]4N4CC(c5c[nH]c6ccccc56)C4)ccc3C2=O)C(=O)N1] | 3-(5-(((1S,2R)-2-(3-(1H- indol-3-yl)azetidin-1-yl)- cyclohexyl)oxy)-1-oxoiso- indolin-2-yl)piperidine-2,6- dione |
| 66-B | 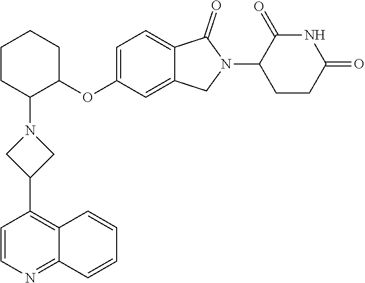 [SMILES: O=C1CCC(N2Cc3cc(OC4CCCCC4N4CC(c5ccnc6ccccc56)C4)ccc3C2=O)C(=O)N1] | 3-(1-oxo-5-((2-(3-(quinolin- 4-yl)azetidin-1-yl)cyclohex- yl)oxy)isoindolin-2-yl)piperi- dine-2,6-dione |
| 67-B | 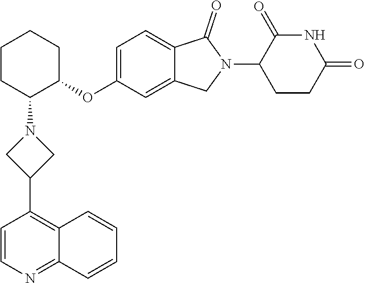 [SMILES: O=C1CCC(N2Cc3cc(O[C@H]4CCCC[C@H]4N4CC(c5ccnc6ccccc56)C4)ccc3C2=O)C(=O)N1] | 3-(1-oxo-5-(((1S,2R)-2-(3- (quinolin-4-yl)azetidin-1-yl)- cyclohexyl)oxy)isoindolin-2- yl)piperidine-2,6-dione |
| 68-B | 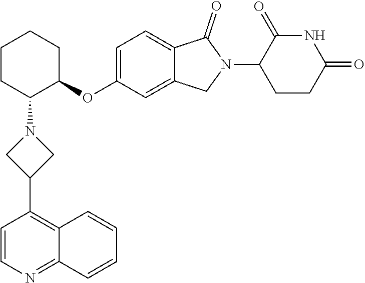 [SMILES: O=C1CCC(N2Cc3cc(O[C@@H]4CCCC[C@H]4N4CC(c5ccnc6ccccc56)C4)ccc3C2=O)C(=O)N1] | 3-(1-oxo-5-(((1R,2R)-2-(3- (quinolin-4-yl)azetidin-1-yl)- cyclohexyl)oxy)isoindolin-2- yl)piperidine-2,6-dione |
| 69-B | 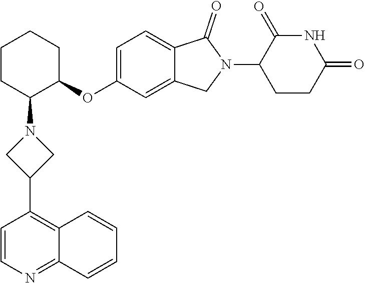 [SMILES: O=C1CCC(N2Cc3cc(O[C@@H]4CCCC[C@@H]4N4CC(c5ccnc6ccccc56)C4)ccc3C2=O)C(=O)N1] | 3-(1-oxo-5-(((1R,2S)-2-(3- (quinolin-4-yl)azetidin-1-yl)- cyclohexyl)oxy)isoindolin-2- yl)piperidine-2,6-dione |
| 70-B | 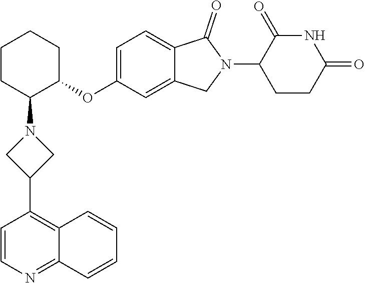 [SMILES: O=C1CCC(N2Cc3cc(O[C@H]4CCCC[C@@H]4N4CC(c5ccnc6ccccc56)C4)ccc3C2=O)C(=O)N1] | 3-(1-oxo-5-(((1S,2S)-2-(3- (quinolin-4-yl)azetidin-1-yl)- cyclohexyl)oxy)isoindolin-2- yl)piperidine-2,6-dione |
| 71-B | 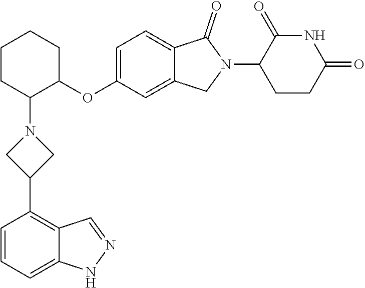 [SMILES: O=C1CCC(N2Cc3cc(OC4CCCCC4N4CC(c5cccc6[nH]ncc56)C4)ccc3C2=O)C(=O)N1] | 3-(5-((2-(3-(1H-indazol-4- yl)azetidin-1-yl)cyclohexyl)- oxy)-1-oxoisoindolin-2-yl)- piperidine-2,6-dione |
| 72-B | 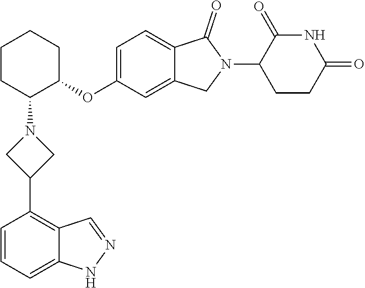 [SMILES: O=C1CCC(N2Cc3cc(O[C@H]4CCCC[C@H]4N4CC(c5cccc6[nH]ncc56)C4)ccc3C2=O)C(=O)N1] | 3-(5-(((1S,2R)-2-(3-(1H- indazol-4-yl)azetidin-1-yl)- cyclohexyl)oxy)-1-oxoiso- indolin-2-yl)piperidine-2,6- dione |
| 73-B | 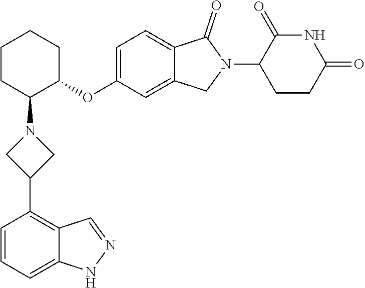 [SMILES: O=C1CCC(N2Cc3cc(O[C@H]4CCCC[C@@H]4N4CC(c5cccc6[nH]ncc56)C4)ccc3C2=O)C(=O)N1] | 3-(5-(((1S,2S)-2-(3-(1H- indazol-4-yl)azetidin-1-yl)- cyclohexyl)oxy)-1-oxoiso- indolin-2-yl)piperidine-2,6- dione |
| 74-B | 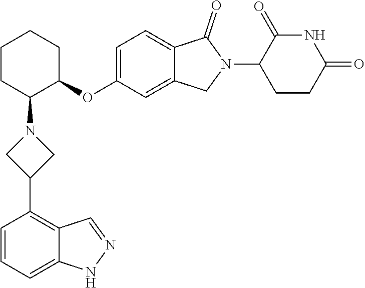 [SMILES: O=C1CCC(N2Cc3cc(O[C@@H]4CCCC[C@@H]4N4CC(c5cccc6[nH]ncc56)C4)ccc3C2=O)C(=O)N1] | 3-(5-(((1R,2S)-2-(3-(1H- indazol-4-yl)azetidin-1-yl)- cyclohexyl)oxy)-1-oxoiso- indolin-2-yl)piperidine-2,6- dione |
| 75-B | 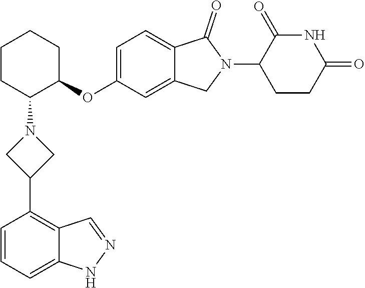 [SMILES: O=C1CCC(N2Cc3cc(O[C@@H]4CCCC[C@H]4N4CC(c5cccc6[nH]ncc56)C4)ccc3C2=O)C(=O)N1] | 3-(5-(((1R,2R)-2-(3-(1H- indazol-4-yl)azetidin-1-yl)- cyclohexyl)oxy)-1-oxoiso- indolin-2-yl)piperidine-2,6- dione |
| 76-B | 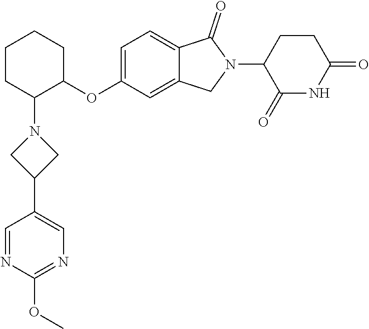 [SMILES: COc1ncc(C2CN(C3CCCCC3Oc3ccc4c(c3)CN(C3CCC(=O)NC3=O)C4=O)C2)cn1] | 3-(5-((2-(3-(2-methoxypyr- imidin-5-yl)azetidin-1-yl)- cyclohexyl)oxy)-1-oxoiso- indolin-2-yl)piperidine-2,6- dione |
| 77-B | 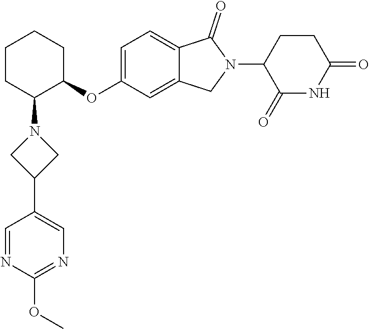 [SMILES: COc1ncc(C2CN([C@H]3CCCC[C@H]3Oc3ccc4c(c3)CN(C3CCC(=O)NC3=O)C4=O)C2)cn1] | 3-(5-(((1R,2S)-2-(3-(2-meth- oxypyrimidin-5-yl)azetidin- 1-yl)cyclohexyl)oxy)-1-oxo- isoindolin-2-yl)piperidine- 2,6-dione |
| 78-B | 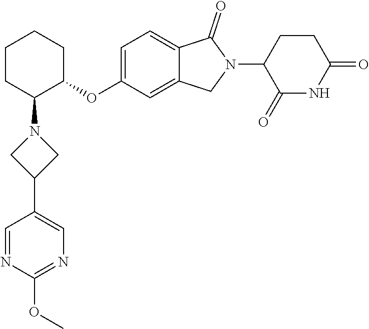 [SMILES: COc1ncc(C2CN([C@H]3CCCC[C@@H]3Oc3ccc4c(c3)CN(C3CCC(=O)NC3=O)C4=O)C2)cn1] | 3-(5-(((1S,2S)-2-(3-(2-meth- oxypyrimidin-5-yl)azetidin- 1-yl)cyclohexyl)oxy)-1-oxo- isoindolin-2-yl)piperidine- 2,6-dione |
| 79-B | 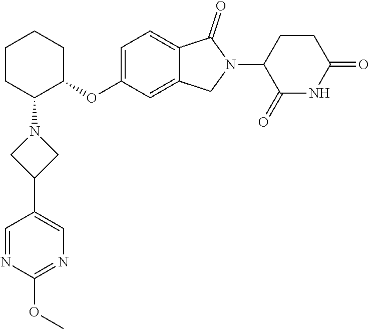 [SMILES: COc1ncc(C2CN([C@@H]3CCCC[C@@H]3Oc3ccc4c(c3)CN(C3CCC(=O)NC3=O)C4=O)C2)cn1] | 3-(5-(((1S,2R)-2-(3-(2-meth- oxypyrimidin-5-yl)azetidin-1- yl)cyclohexyl)oxy)-1-oxo- isoindolin-2-yl)piperidine- 2,6-dione |
| 80-B | 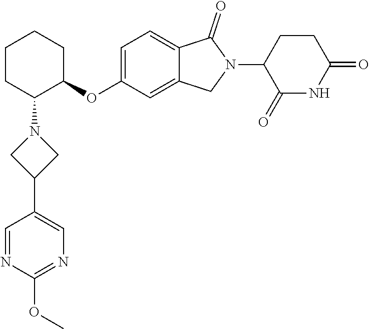 [SMILES: COc1ncc(C2CN([C@@H]3CCCC[C@H]3Oc3ccc4c(c3)CN(C3CCC(=O)NC3=O)C4=O)C2)cn1] | 3-(5-(((1R,2R)-2-(3-(2-meth- oxypyrimidin-5-yl)azetidin- 1-yl)cyclohexyl)oxy)-1-oxo- isoindolin-2-yl)piperidine- 2,6-dione |
| 81-B | 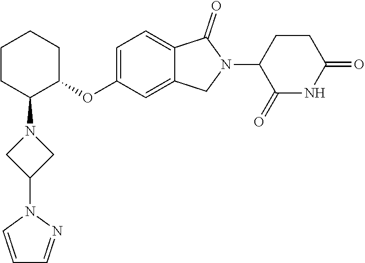 [SMILES: O=C1CCC(N2Cc3cc(O[C@H]4CCCC[C@@H]4N4CC(n5cccn5)C4)ccc3C2=O)C(=O)N1] | 3-(5-(((1S,2S)-2-(3-(1H-pyr- azol-1-yl)azetidin-1-yl)cyclo- hexyl)oxy)-1-oxoisoindolin- 2-yl)piperidine-2,6-dione |
| 82-B | 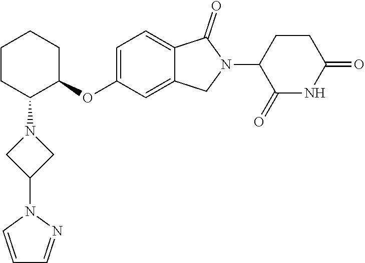 [SMILES: O=C1CCC(N2Cc3cc(O[C@@H]4CCCC[C@H]4N4CC(n5cccn5)C4)ccc3C2=O)C(=O)N1] | 3-(5-(((1R,2R)-2-(3-(1H-pyr- azol-1-yl)azetidin-1-yl)cyclo- hexyl)oxy)-1-oxoisoindolin- 2-yl)piperidine-2,6-dione |
| 83-B | 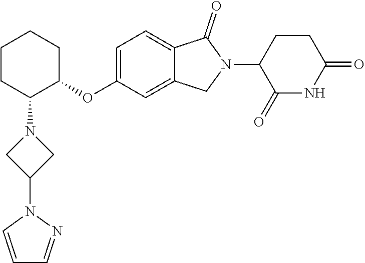 [SMILES: O=C1CCC(N2Cc3cc(O[C@H]4CCCC[C@H]4N4CC(n5cccn5)C4)ccc3C2=O)C(=O)N1] | 3-(5-(((1S,2R)-2-(3-(1H-pyr- azol-1-yl)azetidin-1-yl)cyclo- hexyl)oxy)-1-oxoisoindolin- 2-yl)piperidine-2,6-dione |
| 84-B | 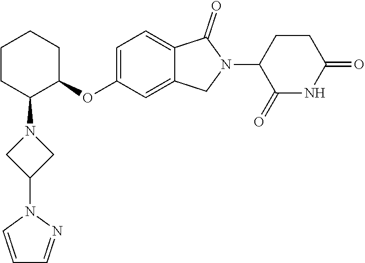 [SMILES: O=C1CCC(N2Cc3cc(O[C@@H]4CCCC[C@@H]4N4CC(n5cccn5)C4)ccc3C2=O)C(=O)N1] | 3-(5-(((1R,2S)-2-(3-(1H-pyr- azol-1-yl)azetidin-1-yl)cyclo- hexyl)oxy)-1-oxoisoindolin- 2-yl)piperidine-2,6-dione |
| 85-B | 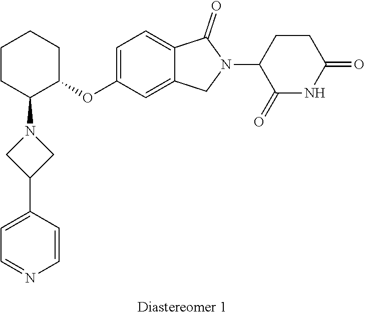 [SMILES: O=C1CCC(N2Cc3cc(O[C@H]4CCCC[C@@H]4N4CC(c5ccncc5)C4)ccc3C2=O)C(=O)N1] | 3-(1-oxo-5-(((1S,2S)-2-(3- (pyridin-4-yl)azetidin-1-yl)- cyclohexyl)oxy)isoindolin-2- yl)piperidine-2,6-dione |
| 86-B | 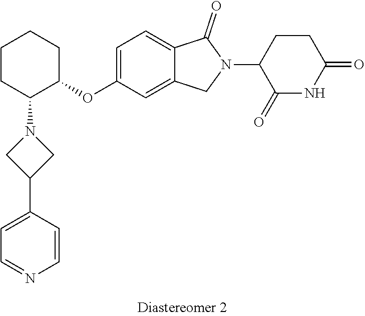 [SMILES: O=C1CCC(N2Cc3cc(O[C@H]4CCCC[C@H]4N4CC(c5ccncc5)C4)ccc3C2=O)C(=O)N1] | 3-(1-oxo-5-(((1S,2R)-2-(3- (pyridin-4-yl)azetidin-1-yl)- cyclohexyl)oxy)isoindolin-2- yl)piperidine-2,6-dione |
| 87-B | 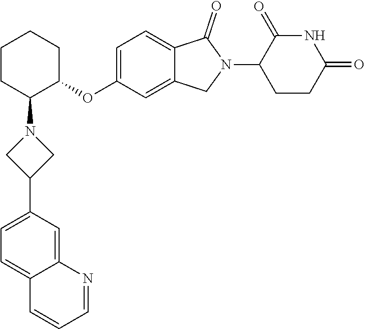 [SMILES: O=C1CCC(N2Cc3cc(O[C@H]4CCCC[C@@H]4N4CC(c5ccc6cccnc6c5)C4)ccc3C2=O)C(=O)N1] | 3-(1-oxo-5-(((1S,2S)-2-(3- (quinolin-7-yl)azetidin-1-yl)- cyclohexyl)oxy)isoindolin-2- yl)piperidine-2,6-dione |
| 88-B | 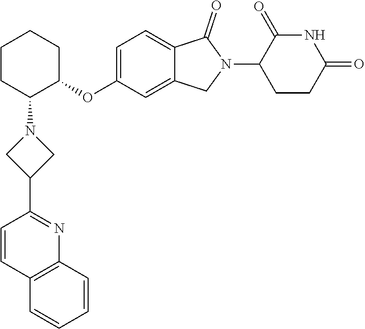 [SMILES: O=C1CCC(N2Cc3cc(O[C@H]4CCCC[C@H]4N4CC(c5ccc6ccccc6n5)C4)ccc3C2=O)C(=O)N1] | 3-(1-oxo-5-(((1S,2R)-2-(3- (quinolin-2-yl)azetidin-1-yl)- cyclohexyl)oxy)isoindolin-2- yl)piperidine-2,6-dione |
| 89-B | 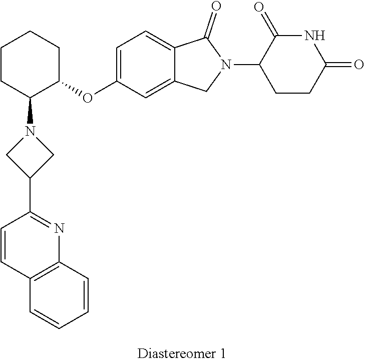 [SMILES: O=C1CCC(N2Cc3cc(O[C@H]4CCCC[C@@H]4N4CC(c5ccc6ccccc6n5)C4)ccc3C2=O)C(=O)N1] | 3-(1-oxo-5-(((1S,2S)-2-(3- (quinolin-2-yl)azetidin-1-yl)- cyclohexyl)oxy)isoindolin-2- yl)piperidine-2,6-dione |
| 90-B | 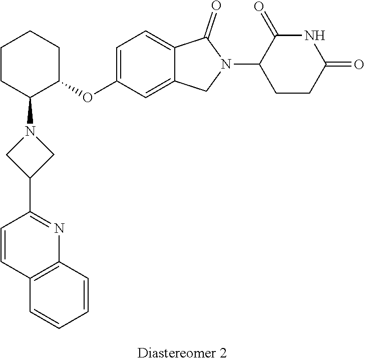 [SMILES: O=C1CCC(N2Cc3cc(O[C@H]4CCCC[C@@H]4N4CC(c5ccc6ccccc6n5)C4)ccc3C2=O)C(=O)N1] | 3-(1-oxo-5-(((1S,2S)-2-(3- (quinolin-2-yl)azetidin-1-yl)- cyclohexyl)oxy)isoindolin-2- yl)piperidine-2,6-dione |
| 91-B | 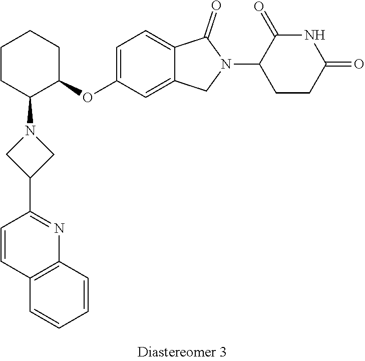 [SMILES: O=C1CCC(N2Cc3cc(O[C@@H]4CCCC[C@@H]4N4CC(c5ccc6ccccc6n5)C4)ccc3C2=O)C(=O)N1] | 3-(1-oxo-5-(((1R,2S)-2-(3- (quinolin-2-yl)azetidin-1- yl)cyclohexyl)oxy)isoindolin- 2-yl)piperidine-2,6-dione |
| 92-B | 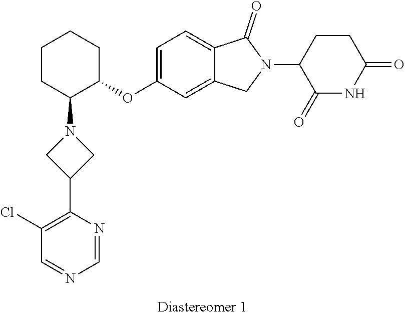 [SMILES: O=C1CCC(N2Cc3cc(O[C@H]4CCCC[C@@H]4N4CC(c5ncncc5Cl)C4)ccc3C2=O)C(=O)N1] | 3-(5-(((1S,2S)-2-(3-(5-chloro- pyrimidin-4-yl)azetidin-1- yl)cyclohexyl)oxy)-1-oxoiso- indolin-2-yl)piperidine-2,6- dione |
| 93-B | 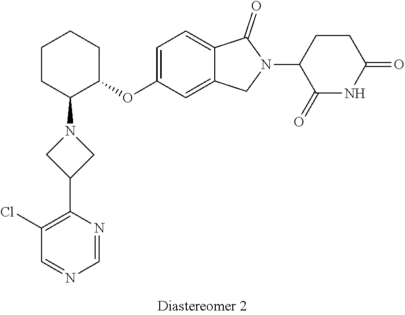 [SMILES: O=C1CCC(N2Cc3cc(O[C@H]4CCCC[C@@H]4N4CC(c5ncncc5Cl)C4)ccc3C2=O)C(=O)N1] | 3-(5-(((1S,2S)-2-(3-(5-chloro- pyrimidin-4-yl)azetidin-1- yl)cyclohexyl)oxy)-1-oxoiso- indolin-2-yl)piperidine-2,6- dione |
| 94-B | 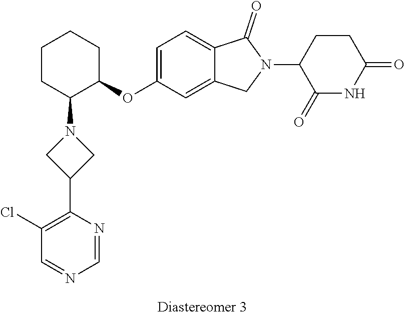 [SMILES: O=C1CCC(N2Cc3cc(O[C@@H]4CCCC[C@@H]4N4CC(c5ncncc5Cl)C4)ccc3C2=O)C(=O)N1] | 3-(5-(((1R,2S)-2-(3-(5-chloro- pyrimidin-4-yl)azetidin-1- yl)cyclohexyl)oxy)-1-oxoiso- indolin-2-yl)piperidine-2,6- dione |
| 95-B | 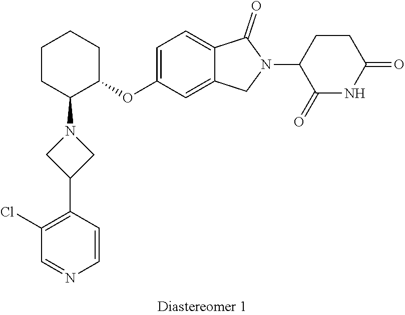 [SMILES: O=C1CCC(N2Cc3cc(O[C@H]4CCCC[C@@H]4N4CC(c5ccncc5Cl)C4)ccc3C2=O)C(=O)N1] | 3-(5-(((1S,2S)-2-(3-(3-chloro- pyridin-4-yl)azetidin-1-yl)- cyclohexyl)oxy)-1-oxoiso- indolin-2-yl)piperidine-2,6- dione |
| 96-B | 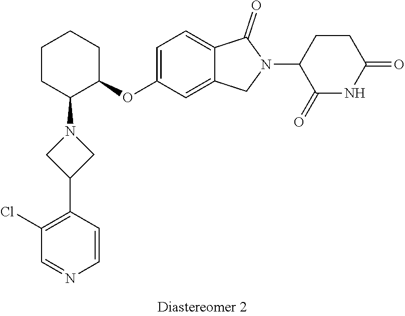 [SMILES: O=C1CCC(N2Cc3cc(O[C@@H]4CCCC[C@@H]4N4CC(c5ccncc5Cl)C4)ccc3C2=O)C(=O)N1] | 3-(5-(((1R,2S)-2-(3-(3-chloro- pyridin-4-yl)azetidin-1-yl)- cyclohexyl)oxy)-1-oxoiso- indolin-2-yl)piperidine-2,6- dione |
| 97-B | 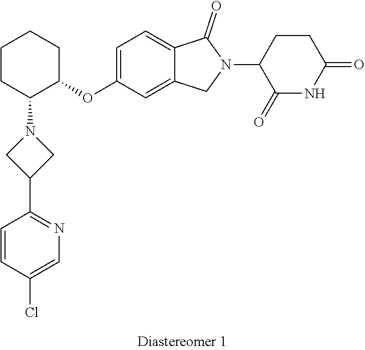 [SMILES: O=C1CCC(N2Cc3cc(O[C@H]4CCCC[C@H]4N4CC(c5ccc(Cl)cn5)C4)ccc3C2=O)C(=O)N1] | 3-(5-(((1S,2R)-2-(3-(5-chloro- pyridin-2-yl)azetidin-1-yl)- cyclohexyl)oxy)-1-oxoiso- indolin-2-yl)piperidine-2,6- dione |
| 98-B | 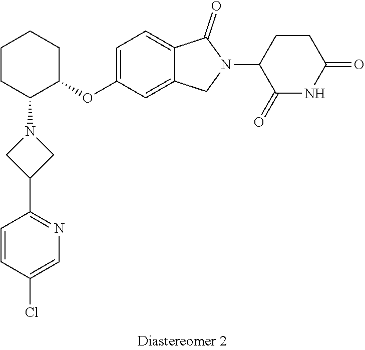 [SMILES: O=C1CCC(N2Cc3cc(O[C@H]4CCCC[C@H]4N4CC(c5ccc(Cl)cn5)C4)ccc3C2=O)C(=O)N1] | 3-(5-(((1S,2R)-2-(3-(5-chloro- pyridin-2-yl)azetidin-1-yl)- cyclohexyl)oxy)-1-oxoiso- indolin-2-yl)piperidine-2,6- dione |
| 99-B | 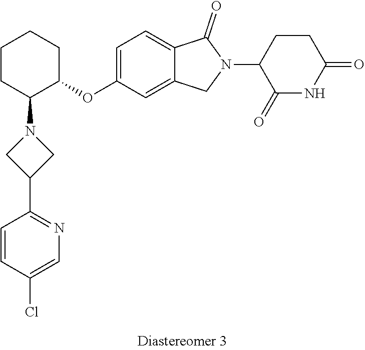 [SMILES: O=C1CCC(N2Cc3cc(O[C@H]4CCCC[C@@H]4N4CC(c5ccc(Cl)cn5)C4)ccc3C2=O)C(=O)N1] | 3-(5-(((1S,2S)-2-(3-(5-chloro- pyridin-2-yl)azetidin-1-yl)- cyclohexyl)oxy)-1-oxoiso- indolin-2-yl)piperidine-2,6- dione |
| 100-B | 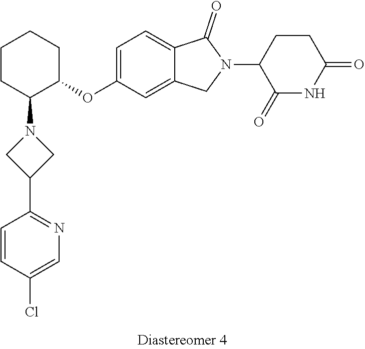 [SMILES: O=C1CCC(N2Cc3cc(O[C@H]4CCCC[C@@H]4N4CC(c5ccc(Cl)cn5)C4)ccc3C2=O)C(=O)N1] | 3-(5-(((1S,2S)-2-(3-(5-chloro- pyridin-2-yl)azetidin-1-yl)- cyclohexyl)oxy)-1-oxoiso- indolin-2-yl)piperidine-2,6- dione |
| 101-B | 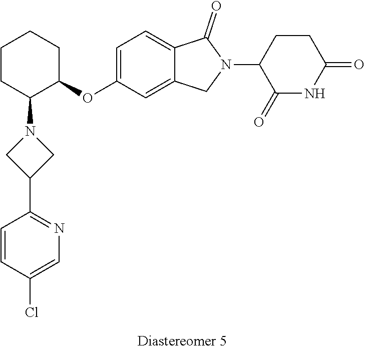 [SMILES: O=C1CCC(N2Cc3cc(O[C@@H]4CCCC[C@@H]4N4CC(c5ccc(Cl)cn5)C4)ccc3C2=O)C(=O)N1] | 3-(5-(((1R,2S)-2-(3-(5-chloro- pyridin-2-yl)azetidin-1-yl)- cyclohexyl)oxy)-1-oxoiso- indolin-2-yl)piperidine-2,6- dione |
| 102-B | 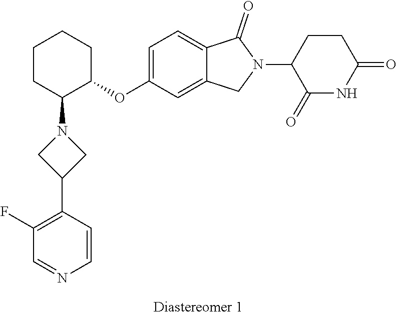 [SMILES: O=C1CCC(N2Cc3cc(O[C@H]4CCCC[C@@H]4N4CC(c5ccncc5F)C4)ccc3C2=O)C(=O)N1] | 3-(5-(((1S,2S)-2-(3-(3-fluoro- pyridin-4-yl)azetidin-1-yl)- cyclohexyl)oxy)-1-oxoiso- indolin-2-yl)piperidine-2,6- dione |
| 103-B | 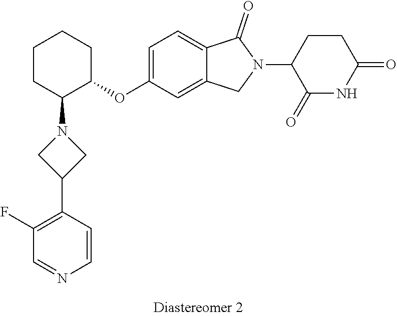 [SMILES: O=C1CCC(N2Cc3cc(O[C@H]4CCCC[C@@H]4N4CC(c5ccncc5F)C4)ccc3C2=O)C(=O)N1] | 3-(5-(((1S,2S)-2-(3-(3-fluoro- pyridin-4-yl)azetidin-1-yl)- cyclohexyl)oxy)-1-oxoiso- indolin-2-yl)piperidine-2,6- dione |
| 104-B | 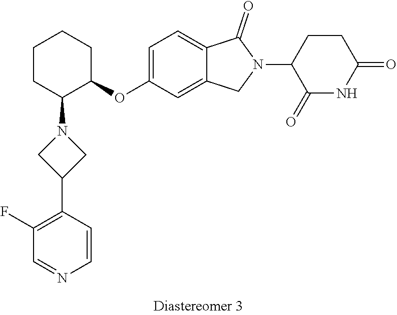 [SMILES: O=C1CCC(N2Cc3cc(O[C@@H]4CCCC[C@@H]4N4CC(c5ccncc5F)C4)ccc3C2=O)C(=O)N1] | 3-(5-(((1R,2S)-2-(3-(3-fluoro- pyridin-4-yl)azetidin-1-yl)- cyclohexyl)oxy)-1-oxoiso- indolin-2-yl)piperidine-2,6- dione |
| 105-B | 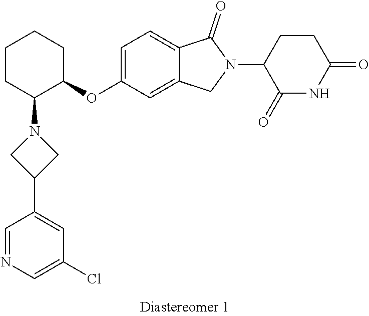 [SMILES: O=C1CCC(N2Cc3cc(O[C@@H]4CCCC[C@@H]4N4CC(c5cncc(Cl)c5)C4)ccc3C2=O)C(=O)N1] | 3-(5-(((1R,2S)-2-(3-(5-chloro- pyridin-3-yl)azetidin-1-yl)- cyclohexyl)oxy)-1-oxoiso- indolin-2-yl)piperidine-2,6- dione |
| 106-B | 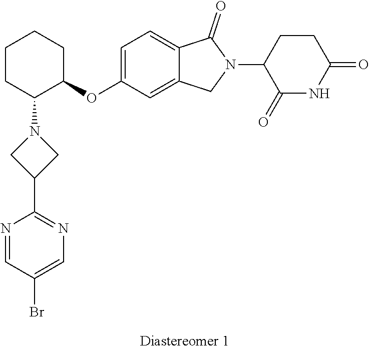 [SMILES: O=C1CCC(N2Cc3cc(O[C@@H]4CCCC[C@H]4N4CC(c5ncc(Br)cn5)C4)ccc3C2=O)C(=O)N1] | 3-(5-(((1R,2R)-2-(3-(5-bromo- pyrimidin-2-yl)azetidin-1- yl)cyclohexyl)oxy)-1-oxoiso- indolin-2-yl)piperidine-2,6- dione |
| 107-B | 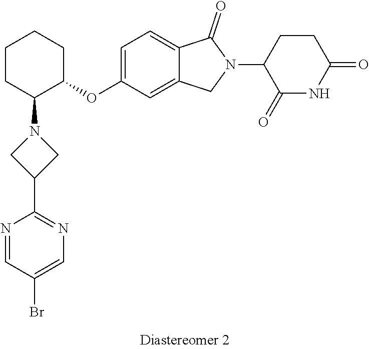 [SMILES: O=C1CCC(N2Cc3cc(O[C@H]4CCCC[C@@H]4N4CC(c5ncc(Br)cn5)C4)ccc3C2=O)C(=O)N1] | 3-(5-(((1S,2S)-2-(3-(5-bromo- pyrimidin-2-yl)azetidin-1-yl)- cyclohexyl)oxy)-1-oxoiso- indolin-2-yl)piperidine-2,6- dione |
| 108-B | 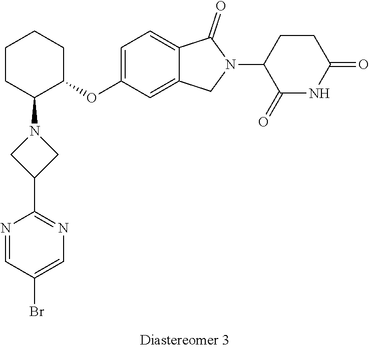 [SMILES: O=C1CCC(N2Cc3cc(O[C@H]4CCCC[C@@H]4N4CC(c5ncc(Br)cn5)C4)ccc3C2=O)C(=O)N1] | 3-(5-(((1S,2S)-2-(3-(5-bromo- pyrimidin-2-yl)azetidin-1- yl)cyclohexyl)oxy)-1-oxoiso- indolin-2-yl)piperidine-2,6- dione |
| 109-B | 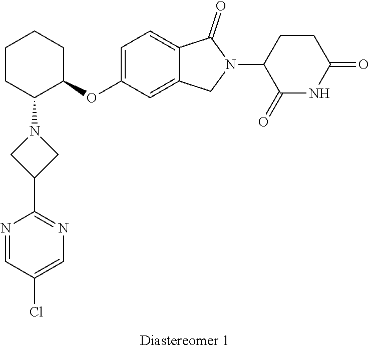 [SMILES: O=C1CCC(N2Cc3cc(O[C@@H]4CCCC[C@H]4N4CC(c5ncc(Cl)cn5)C4)ccc3C2=O)C(=O)N1] | 3-(5-(((1R,2R)-2-(3-(5-chloro- pyrimidin-2-yl)azetidin-1-yl)- cyclohexyl)oxy)-1-oxoiso- indolin-2-yl)piperidine-2,6- dione |
| 110-B | 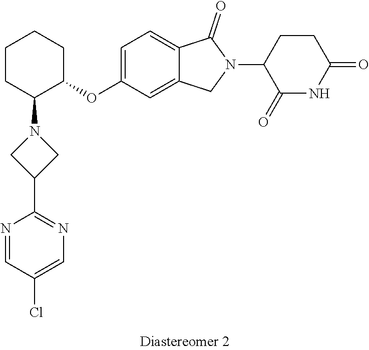 [SMILES: O=C1CCC(N2Cc3cc(O[C@H]4CCCC[C@@H]4N4CC(c5ncc(Cl)cn5)C4)ccc3C2=O)C(=O)N1] | 3-(5-(((1S,2S)-2-(3-(5-chloro- pyrimidin-2-yl)azetidin-1-yl)- cyclohexyl)oxy)-1-oxoiso- indolin-2-yl)piperidine-2,6- dione |
| 111-B | 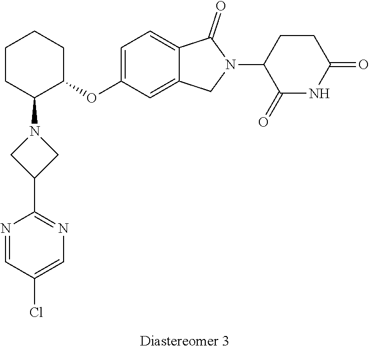 [SMILES: O=C1CCC(N2Cc3cc(O[C@H]4CCCC[C@@H]4N4CC(c5ncc(Cl)cn5)C4)ccc3C2=O)C(=O)N1] | 3-(5-(((1S,2S)-2-(3-(5-chloro- pyrimidin-2-yl)azetidin-1-yl)- cyclohexyl)oxy)-1-oxoiso- indolin-2-yl)piperidine-2,6- dione |
| 112-B | 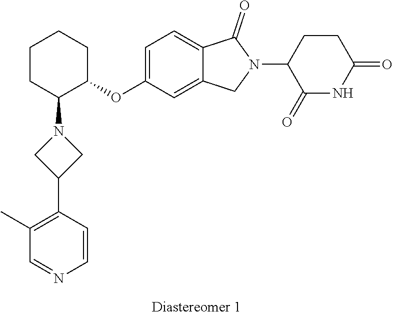 [SMILES: Cc1cnccc1C1CN([C@H]2CCCC[C@@H]2Oc2ccc3c(c2)CN(C2CCC(=O)NC2=O)C3=O)C1] | 3-(5-(((1S,2S)-2-(3-(3-meth- ylpyridin-4-yl)azetidin-1- yl)cyclohexyl)oxy)-1-oxoiso- indolin-2-yl)piperidine-2,6- dione |
| 113-B | 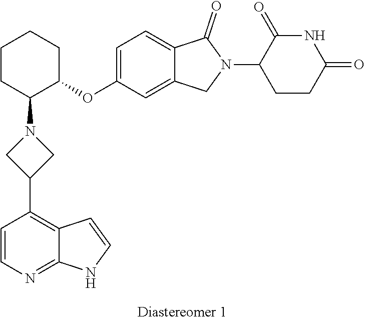 [SMILES: O=C1CCC(N2Cc3cc(O[C@H]4CCCC[C@@H]4N4CC(c5ccnc6[nH]ccc56)C4)ccc3C2=O)C(=O)N1] | 3-(5-(((1S,2S)-2-(3-(1H-pyr- rolo[2,3-b]pyridin-4-yl)azet- idin-1-yl)cyclohexyl)oxy)-1- oxoisoindolin-2-yl)piperidine- 2,6-dione |
| 114-B | 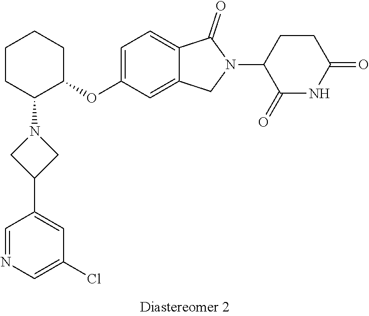 [SMILES: O=C1CCC(N2Cc3cc(O[C@H]4CCCC[C@H]4N4CC(c5cncc(Cl)c5)C4)ccc3C2=O)C(=O)N1] | 3-(5-(((1S,2R)-2-(3-(5-chloro- pyridin-3-yl)azetidin-1-yl)- cyclohexyl)oxy)-1-oxoiso- indolin-2-yl)piperidine-2,6- dione |
| 115-B | 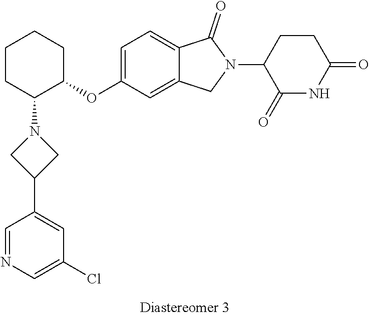 [SMILES: O=C1CCC(N2Cc3cc(O[C@H]4CCCC[C@H]4N4CC(c5cncc(Cl)c5)C4)ccc3C2=O)C(=O)N1] | 3-(5-(((1S,2R)-2-(3-(5-chloro- pyridin-3-yl)azetidin-1-yl)- cyclohexyl)oxy)-1-oxoiso- indolin-2-yl)piperidine-2,6- dione |
| 116-B | 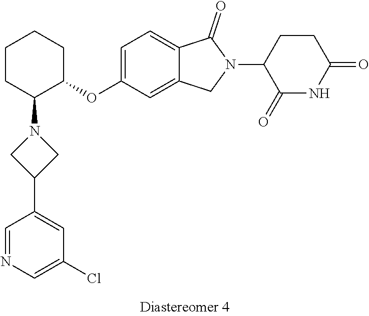 [SMILES: O=C1CCC(N2Cc3cc(O[C@H]4CCCC[C@@H]4N4CC(c5cncc(Cl)c5)C4)ccc3C2=O)C(=O)N1] | 3-(5-(((1S,2S)-2-(3-(5-chloro- pyridin-3-yl)azetidin-1-yl)- cyclohexyl)oxy)-1-oxoiso- indolin-2-yl)piperidine-2,6- dione |
| 117-B | 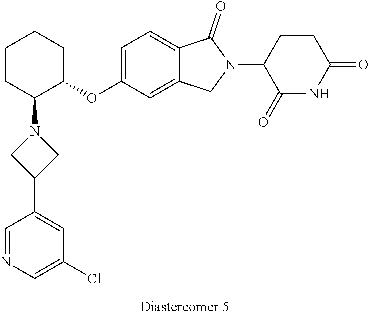 [SMILES: O=C1CCC(N2Cc3cc(O[C@H]4CCCC[C@@H]4N4CC(c5cncc(Cl)c5)C4)ccc3C2=O)C(=O)N1] | 3-(5-(((1S,2S)-2-(3-(5-chloro- pyridin-3-yl)azetidin-1-yl)- cyclohexyl)oxy)-1-oxoiso- indolin-2-yl)piperidine-2,6- dione |
| 118-B | 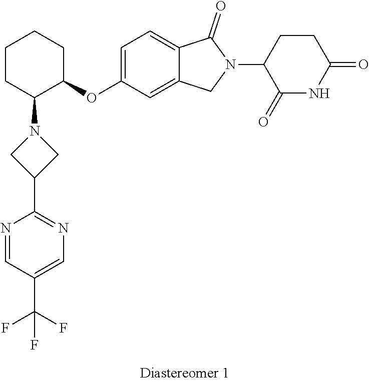 [SMILES: O=C1CCC(N2Cc3cc(O[C@@H]4CCCC[C@@H]4N4CC(c5ncc(C(F)(F)F)cn5)C4)ccc3C2=O)C(=O)N1] | 3-(1-oxo-5-(((1R,2S)-2-(3-(5- (trifluoromethyl)pyrimidin-2- yl)azetidin-1-yl)cyclohexyl)- oxy)isoindolin-2-yl)piperi- dine-2,6-dione |
| 119-B | 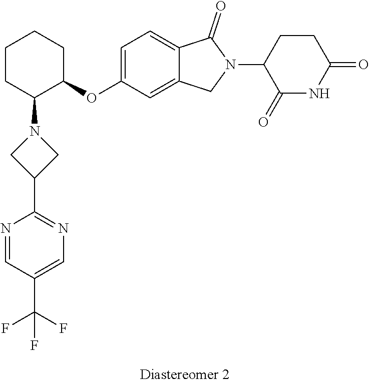 [SMILES: O=C1CCC(N2Cc3cc(O[C@@H]4CCCC[C@@H]4N4CC(c5ncc(C(F)(F)F)cn5)C4)ccc3C2=O)C(=O)N1] | 3-(1-oxo-5-(((1R.2S)-2-(3-(5- (trifluoromethyl)pyrimidin-2- yl)azetidin-1-yl)cyclohexyl)- oxy)isoindolin-2-yl)piperi- dine-2,6-dione |
| 120-B | 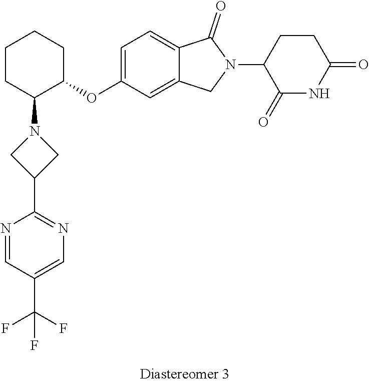 [SMILES: O=C1CCC(N2Cc3cc(O[C@H]4CCCC[C@@H]4N4CC(c5ncc(C(F)(F)F)cn5)C4)ccc3C2=O)C(=O)N1] | 3-(1-oxo-5-(((1S,2S)-2-(3-(5- (trifluoromethyl)pyrimidin-2- yl)azetidin-1-yl)cyclohexyl)- oxy)isoindolin-2-yl)piperidine- 2,6-dione |
| 121-B | 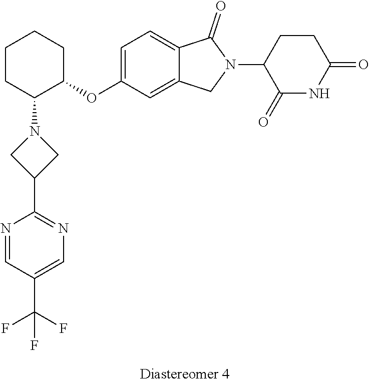 [SMILES: O=C1CCC(N2Cc3cc(O[C@H]4CCCC[C@H]4N4CC(c5ncc(C(F)(F)F)cn5)C4)ccc3C2=O)C(=O)N1] | 3-(1-oxo-5-(((1S,2R)-2-(3-(5- (trifluoromethyl)pyrimidin-2- yl)azetidin-1-yl)cyclohexyl)- oxy)isoindolin-2-yl)piperi- dine-2,6-dione |
| 122-B | 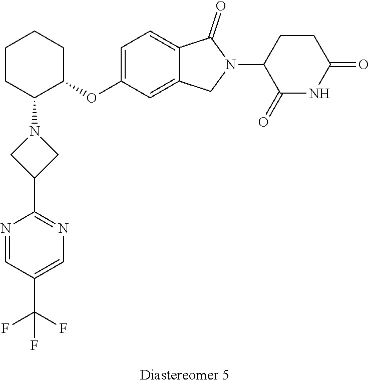 [SMILES: O=C1CCC(N2Cc3cc(O[C@H]4CCCC[C@H]4N4CC(c5ncc(C(F)(F)F)cn5)C4)ccc3C2=O)C(=O)N1] | 3-(1-oxo-5-(((1S,2R)-2-(3-(5- (trifluoromethyl)pyrimidin-2- yl)azetidin-1-yl)cyclohexyl)- oxy)isoindolin-2-yl)piperi- dine-2,6-dione |
| 123-B | 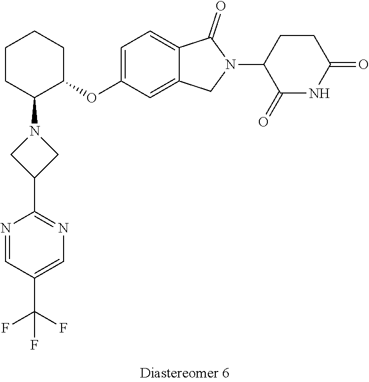 [SMILES: O=C1CCC(N2Cc3cc(O[C@H]4CCCC[C@@H]4N4CC(c5ncc(C(F)(F)F)cn5)C4)ccc3C2=O)C(=O)N1] | 3-(1-oxo-5-(((1S,2S)-2-(3-(5- (trifluoromethyl)pyrimidin-2- yl)azetidin-1-yl)cyclohexyl)- oxy)isoindolin-2-yl)piperi- dine-2,6-dione |
| 124-B | 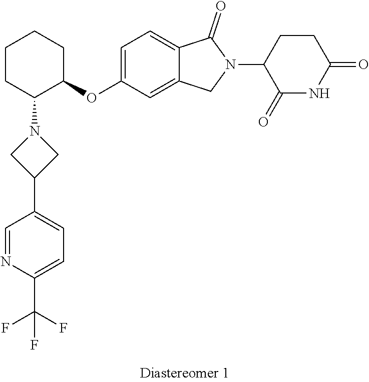 [SMILES: O=C1CCC(N2Cc3cc(O[C@@H]4CCCC[C@H]4N4CC(c5ccc(C(F)(F)F)nc5)C4)ccc3C2=O)C(=O)N1] | 3-(1-oxo-5-(((1R,2R)-2-(3- (6-(trifluoromethyl)pyridin-3- yl)azetidin-1-yl)cyclohexyl)- oxy)isoindolin-2-yl)piperi- dine-2,6-dione |
| 125-B | 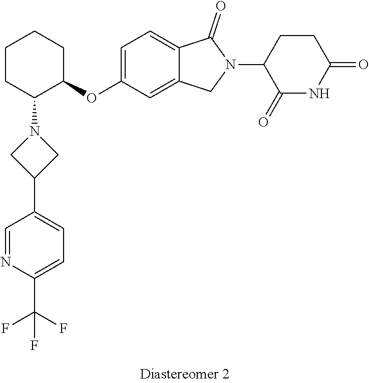 [SMILES: O=C1CCC(N2Cc3cc(O[C@@H]4CCCC[C@H]4N4CC(c5ccc(C(F)(F)F)nc5)C4)ccc3C2=O)C(=O)N1] | 3-(1-oxo-5-(((1R,2R)-2-(3-(6- (trifluoromethyl)pyridin-3- yl)azetidin-1-yl)cyclohexyl)- oxy)isoindolin-2-yl)piperi- dine-2,6-dione |
| 126-B | 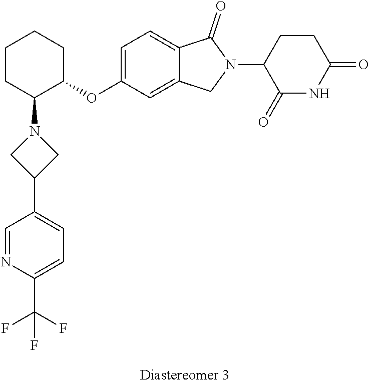 [SMILES: O=C1CCC(N2Cc3cc(O[C@H]4CCCC[C@@H]4N4CC(c5ccc(C(F)(F)F)nc5)C4)ccc3C2=O)C(=O)N1] | 3-(1-oxo-5-(((1S,2S)-2-(3-(6- (trifluoromethyl)pyridin-3- yl)azetidin-1-yl)cyclohexyl)- oxy)isoindolin-2-yl)piperi- dine-2,6-dione |
| 127-B | 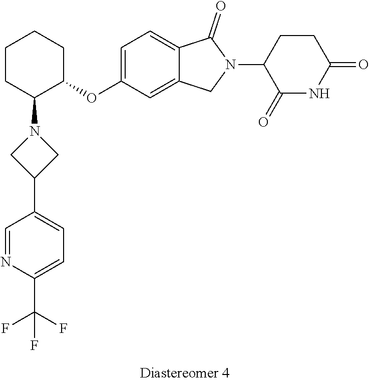 [SMILES: O=C1CCC(N2Cc3cc(O[C@H]4CCCC[C@@H]4N4CC(c5ccc(C(F)(F)F)nc5)C4)ccc3C2=O)C(=O)N1] | 3-(1-oxo-5-(((1S,2S)-2-(3-(6- (trifluoromethyl)pyridin-3- yl)azetidin-1-yl)cyclohexyl)- oxy)isoindolin-2-yl)piperi- dine-2,6-dione |
| 128-B | 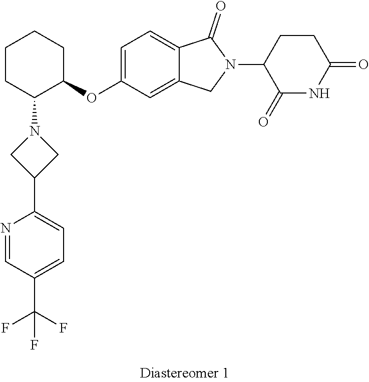 [SMILES: O=C1CCC(N2Cc3cc(O[C@@H]4CCCC[C@H]4N4CC(c5ccc(C(F)(F)F)cn5)C4)ccc3C2=O)C(=O)N1] | 3-(1-oxo-5-(((1R,2R)-2-(3-(5- (trifluoromethyl)pyridin-2- yl)azetidin-1-yl)cyclohexyl)- oxy)isoindolin-2-yl)piperi- dine-2,6-dione |
| 129-B | 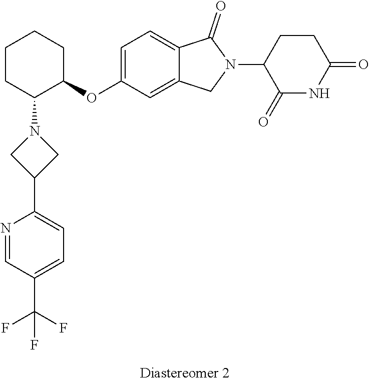 [SMILES: O=C1CCC(N2Cc3cc(O[C@@H]4CCCC[C@H]4N4CC(c5ccc(C(F)(F)F)cn5)C4)ccc3C2=O)C(=O)N1] | 3-(1-oxo-5-(((1R.2R)-2-(3-(5- (trifluoromethyl)pyridin-2- yl)azetidin-1-yl)cyclohexyl)- oxy)isoindolin-2-yl)piperi- dine-2,6-dione |
| 130-B | 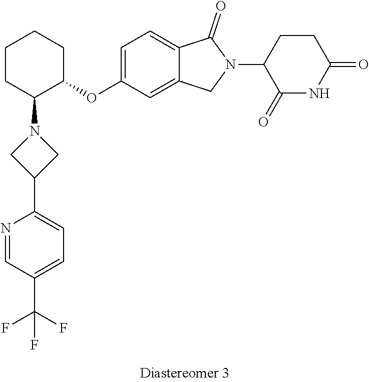 [SMILES: O=C1CCC(N2Cc3cc(O[C@H]4CCCC[C@@H]4N4CC(c5ccc(C(F)(F)F)cn5)C4)ccc3C2=O)C(=O)N1] | 3-(1-oxo-5-(((1S,2S)-2-(3-(5- (trifluoromethyl)pyridin-2- yl)azetidin-1-yl)cyclohexyl)- oxy)isoindolin-2-yl)piperi- dine-2,6-dione |
| 131-B | 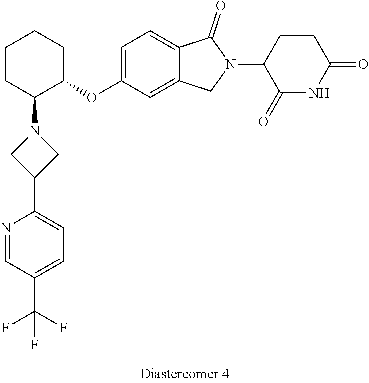 [SMILES: O=C1CCC(N2Cc3cc(O[C@H]4CCCC[C@@H]4N4CC(c5ccc(C(F)(F)F)cn5)C4)ccc3C2=O)C(=O)N1] | 3-(1-oxo-5-(((1S,2S)-2-(3-(5- (trifluoromethyl)pyridin-2- yl)azetidin-1-yl)cyclohexyl)- oxy)isoindolin-2-yl)piperi- dine-2,6-dione |
| 132-B | 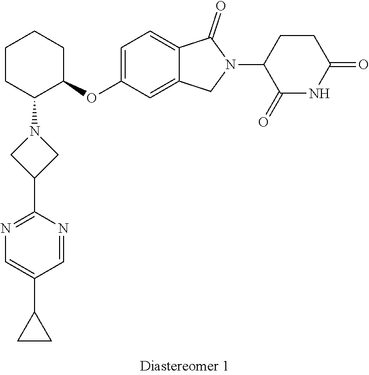 [SMILES: O=C1CCC(N2Cc3cc(O[C@@H]4CCCC[C@H]4N4CC(c5ncc(C6CC6)cn5)C4)ccc3C2=O)C(=O)N1] | 3-(5-(((1R,2R)-2-(3-(5-cyclo- propylpyrimidin-2-yl)azetidin- 1-yl)cyclohexyl)oxy)-1-oxo- isoindolin-2-yl)piperidine-2,6- dione |
| 133-B | 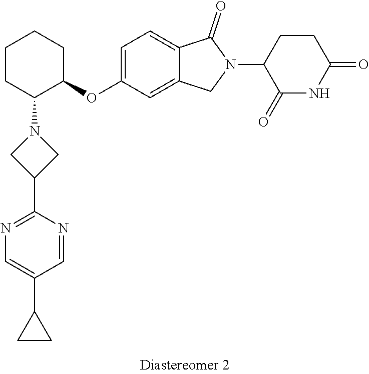 [SMILES: O=C1CCC(N2Cc3cc(O[C@@H]4CCCC[C@H]4N4CC(c5ncc(C6CC6)cn5)C4)ccc3C2=O)C(=O)N1] | 3-(5-(((1R,2R)-2-(3-(5-cyclo- propylpyrimidin-2-yl)azetidin- 1-yl)cyclohexyl)oxy)-1-oxo- isoindolin-2-yl)piperidine-2,6- dione |
| 134-B | 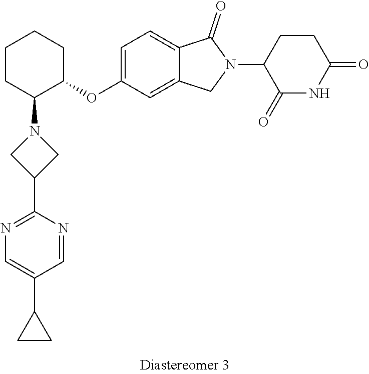 [SMILES: O=C1CCC(N2Cc3cc(O[C@H]4CCCC[C@@H]4N4CC(c5ncc(C6CC6)cn5)C4)ccc3C2=O)C(=O)N1] | 3-(5-(((1S,2S)-2-(3-(5-cyclo- propylpyrimidin-2-yl)azetidin- 1-yl)cyclohexyl)oxy)-1-oxo- isoindolin-2-yl)piperidine-2,6- dione |
| 135-B | 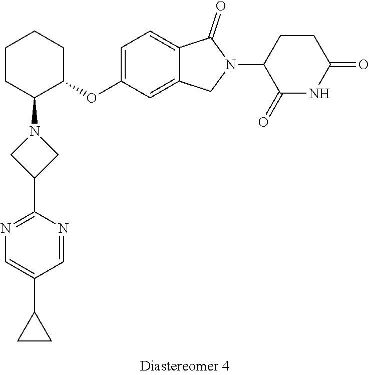 [SMILES: O=C1CCC(N2Cc3cc(O[C@H]4CCCC[C@@H]4N4CC(c5ncc(C6CC6)cn5)C4)ccc3C2=O)C(=O)N1] | 3-(5-(((1S,2S)-2-(3-(5-cyclo- propylpyrimidin-2-yl)azetidin- 1-yl)cyclohexyl)oxy)-1-oxo- isoindolin-2-yl)piperidine-2,6- dione |
| 136-B | 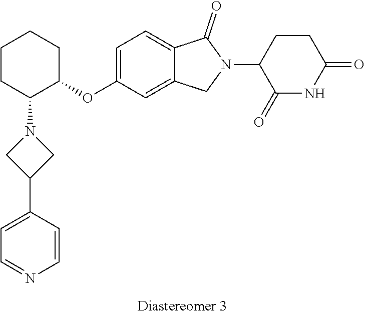 [SMILES: O=C1CCC(N2Cc3cc(O[C@H]4CCCC[C@H]4N4CC(c5ccncc5)C4)ccc3C2=O)C(=O)N1] | 3-(1-oxo-5-(((1S,2R)-2-(3- (pyridin-4-yl)azetidin-1-yl)- cyclohexyl)oxy)isoindolin-2- yl)piperidine-2,6-dione |
| 137-B | 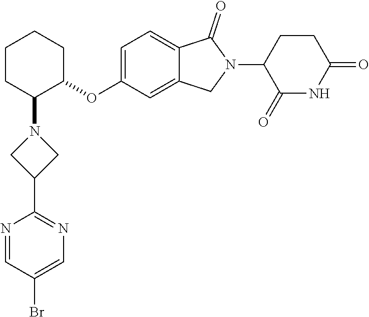 [SMILES: O=C1CCC(N2Cc3cc(O[C@H]4CCCC[C@@H]4N4CC(c5ncc(Br)cn5)C4)ccc3C2=O)C(=O)N1] | Rac-3-(5-(((trans)-2-(3-(5- bromopyrimidin-2-yl)azetidin- 1-yl)cyclohexyl)oxy)-1-oxo- isoindolin-2-yl)piperidine-2,6- dione |
| 138-B | 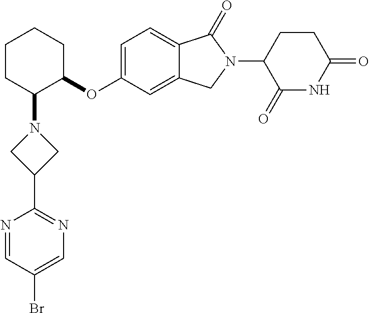 [SMILES: O=C1CCC(N2Cc3cc(O[C@@H]4CCCC[C@@H]4N4CC(c5ncc(Br)cn5)C4)ccc3C2=O)C(=O)N1] | Rac-3-(5-(((cis)-2-(3-(5- bromopyrimidin-2-yl)azetidin- 1-yl)cyclohexyl)oxy)-1-oxo- isoindolin-2-yl)piperidine-2,6- dione |
| 139-B | 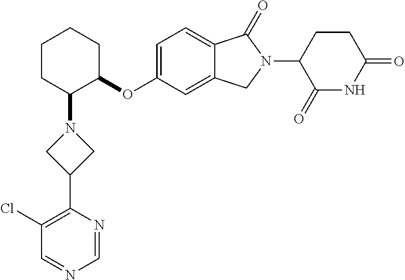 [SMILES: O=C1CCC(N2Cc3cc(O[C@@H]4CCCC[C@@H]4N4CC(c5ncncc5Cl)C4)ccc3C2=O)C(=O)N1] | Rac-3-(5-(((cis)-2-(3-(5- chloropyrimidin-4-yl)azetidin- 1-yl)cyclohexyl)oxy)-1-oxo- isoindolin-2-yl)piperidine-2,6- dione |
| 140-B | 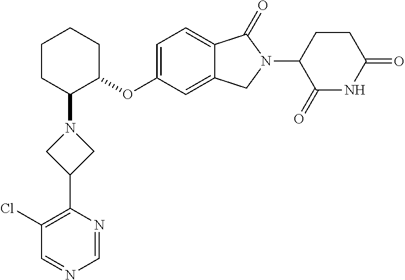 [SMILES: O=C1CCC(N2Cc3cc(O[C@H]4CCCC[C@@H]4N4CC(c5ncncc5Cl)C4)ccc3C2=O)C(=O)N1] | Rac-3-(5-(((trans)-2-(3-(5- chloropyrimidin-4-yl)azetidin- 1-yl)cyclohexyl)oxy)-1-oxo- isoindolin-2-yl)piperidine-2,6- dione |
| 141-B | 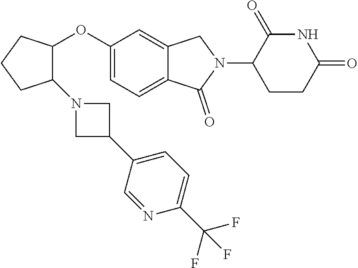 [SMILES: O=C1CCC(N2Cc3cc(OC4CCCC4N4CC(c5ccc(C(F)(F)F)nc5)C4)ccc3C2=O)C(=O)N1] | 3-(1-oxo-5-((2-(3-(6-(trifluoro- methyl)pyridin-3-yl)azetidin- 1-yl)cyclopentyl)oxy)iso- indolin-2-yl)piperidine-2,6- dione |
| 142-B | 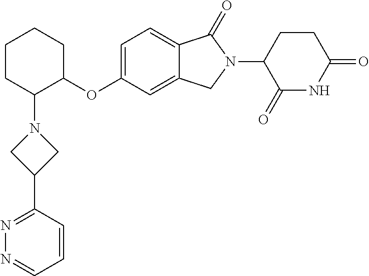 [SMILES: O=C1CCC(N2Cc3cc(OC4CCCCC4N4CC(c5cccnn5)C4)ccc3C2=O)C(=O)N1] | 3-(1-oxo-5-((2-(3-(pyridazin- 3-yl)azetidin-1-yl)cyclohex- yl)oxy)isoindolin-2-yl)piperi- dine-2,6-dione |
| 143-B | 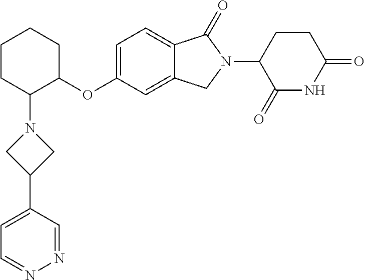 [SMILES: O=C1CCC(N2Cc3cc(OC4CCCCC4N4CC(c5ccnnc5)C4)ccc3C2=O)C(=O)N1] | 3-(1-oxo-5-((2-(3-(pyridazin- 4-yl)azetidin-1-yl)cyclohex- yl)oxy)isoindolin-2-yl)piperi- dine-2,6-dione |
| 144-B | 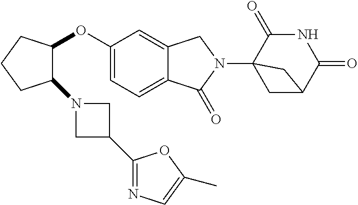 [SMILES: Cc1cnc(C2CN([C@H]3CCC[C@H]3Oc3ccc4c(c3)CN(C35CC(C3)C(=O)NC5=O)C4=O)C2)o1] | Rac-1-(5-(((cis)-2-(3-(5-meth- yloxazol-2-yl)azetidin-1-yl)- cyclopentyl)oxy)-1-oxoiso- indolin-2-yl)-3-azabicyclo [3.1.1]heptane-2,4-dione |
| 145-B | 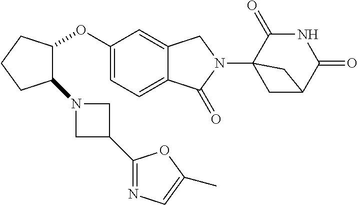 [SMILES: Cc1cnc(C2CN([C@H]3CCC[C@@H]3Oc3ccc4c(c3)CN(C35CC(C3)C(=O)NC5=O)C4=O)C2)o1] | Rac-1-(5-(((trans)-2-(3-(5- methyloxazol-2-yl)azetidin-1- yl)cyclopentyl)oxy)-1-oxoiso- indolin-2-yl)-3-azabicyclo [3.1.1]heptane-2,4-dione |
| 146-B | 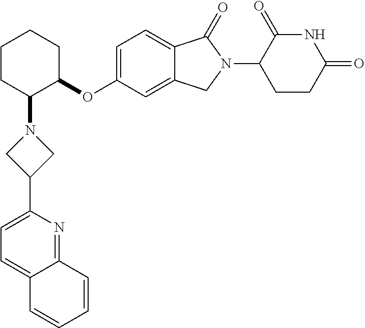 [SMILES: O=C1CCC(N2Cc3cc(O[C@@H]4CCCC[C@@H]4N4CC(c5ccc6ccccc6n5)C4)ccc3C2=O)C(=O)N1] | Rac-3-(1-oxo-5-(((cis)-2-(3- (quinolin-2-yl)azetidin-1-yl)- cyclohexyl)oxy)isoindolin-2- yl)piperidine-2,6-dione |
| 147-B | 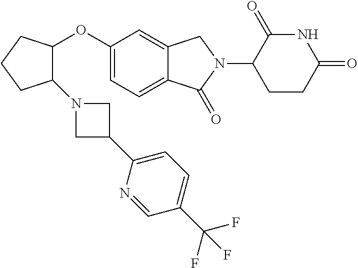 [SMILES: O=C1CCC(N2Cc3cc(OC4CCCC4N4CC(c5ccc(C(F)(F)F)cn5)C4)ccc3C2=O)C(=O)N1] | 3-(1-oxo-5-((2-(3-(5-(trifluoro- methyl)pyridin-2-yl)azetidin- 1-yl)cyclopentyl)oxy)iso- indolin-2-yl)piperidine-2,6- dione |
| 148-B | 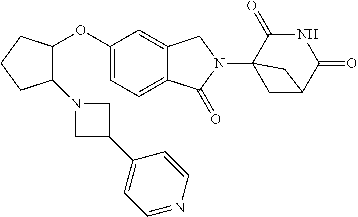 [SMILES: O=C1NC(=O)C2(N3Cc4cc(OC5CCCC5N5CC(c6ccncc6)C5)ccc4C3=O)CC1C2] | 1-(1-oxo-5-((2-(3-(pyridin-4- yl)azetidin-1-yl)cyclopentyl)- oxy)isoindolin-2-yl)-3-aza- bicyclo[3.1.1]heptane-2,4- dione |
| 149-B | 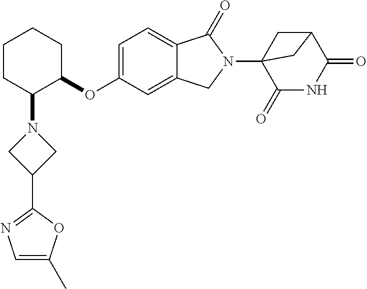 [SMILES: Cc1cnc(C2CN([C@H]3CCCC[C@H]3Oc3ccc4c(c3)CN(C35CC(C3)C(=O)NC5=O)C4=O)C2)o1] | Rac-1-(5-(((cis)-2-(3-(5-meth- yloxazol-2-yl)azetidin-1-yl)- cyclohexyl)oxy)-1-oxoiso- indolin-2-yl)-3-azabicyclo [3.1.1]heptane-2,4-dione |
| 150-B | 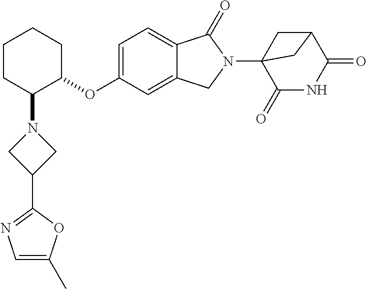 [SMILES: Cc1cnc(C2CN([C@H]3CCCC[C@@H]3Oc3ccc4c(c3)CN(C35CC(C3)C(=O)NC5=O)C4=O)C2)o1] | Rac-1-(5-(((trans)-2-(3-(5- methyloxazol-2-yl)azetidin-1- yl)cyclohexyl)oxy)-1-oxoiso- indolin-2-yl)-3-azabicyclo [3.1.1]heptane-2,4-dione |
| 151-B | 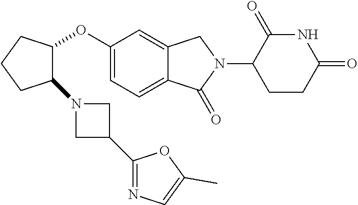 [SMILES: Cc1cnc(C2CN([C@H]3CCC[C@@H]3Oc3ccc4c(c3)CN(C3CCC(=O)NC3=O)C4=O)C2)o1] | Rac-3-(5-(((trans)-2-(3-(5- methyloxazol-2-yl)azetidin-1- yl)cyclopentyl)oxy)-1-oxoiso- indolin-2-yl)piperidine-2,6- dione |
| 152-B | 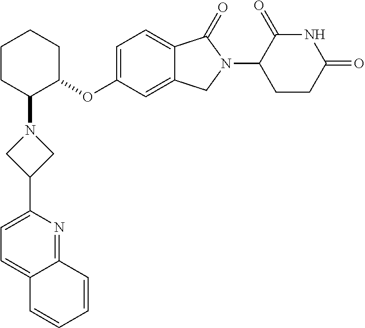 [SMILES: O=C1CCC(N2Cc3cc(O[C@H]4CCCC[C@@H]4N4CC(c5ccc6ccccc6n5)C4)ccc3C2=O)C(=O)N1] | Rac-3-(1-oxo-5-(((trans)-2-(3- (quinolin-2-yl)azetidin-1- yl)cyclohexyl)oxy)isoindolin- 2-yl)piperidine-2,6-dione |
| 153-B | 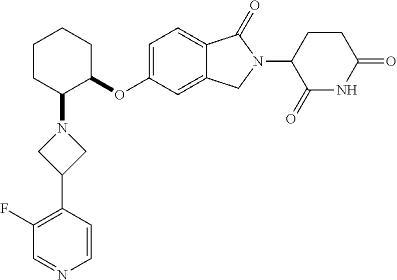 [SMILES: O=C1CCC(N2Cc3cc(O[C@@H]4CCCC[C@@H]4N4CC(c5ccncc5F)C4)ccc3C2=O)C(=O)N1] | Rac-3-(5-(((cis)-2-(3-(3-fluoro- pyridin-4-yl)azetidin-1-yl)- cyclohexyl)oxy)-1-oxoiso- indolin-2-yl)piperidine-2,6- dione |
| 154-B | 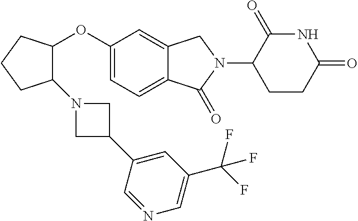 [SMILES: O=C1CCC(N2Cc3cc(OC4CCCC4N4CC(c5cncc(C(F)(F)F)c5)C4)ccc3C2=O)C(=O)N1] | 3-(1-oxo-5-((2-(3-(5-(trifluoro- methyl)pyridin-3-yl)azetidin- 1-yl)cyclopentyl)oxy)iso- indolin-2-yl)piperidine-2,6- dione |
| 155-B | 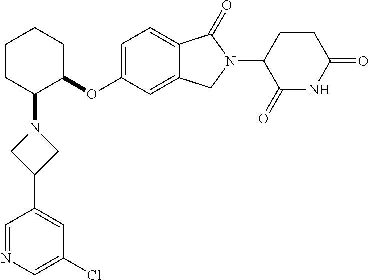 [SMILES: O=C1CCC(N2Cc3cc(O[C@@H]4CCCC[C@@H]4N4CC(c5cncc(Cl)c5)C4)ccc3C2=O)C(=O)N1] | Rac-3-(5-(((cis)-2-(3-(5- chloropyridin-3-yl)azetidin-1- yl)cyclohexyl)oxy)-1-oxoiso- indolin-2-yl)piperidine-2,6- dione |
| 156-B | 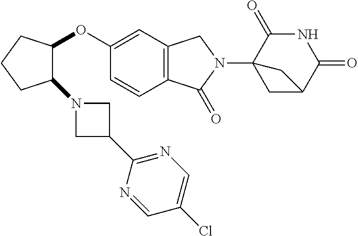 | Rac-1-(5-(((cis)-2-(3-(5- chloropyrimidin-2-yl)azetidin- 1-yl)cyclopentyl)oxy)-1-oxo- isoindolin-2-yl)-3-azabicyclo [3.1.1]heptane-2,4-dione |
| 157-B | 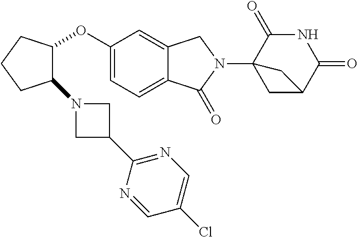 | Rac-1-(5-(((trans)-2-(3-(5- chloropyrimidin-2-yl)azetidin- 1-yl)cyclopentyl)oxy)-1-oxo- isoindolin-2-yl)-3-azabicyclo [3.1.1]heptane-2,4-dione |
| 158-B | 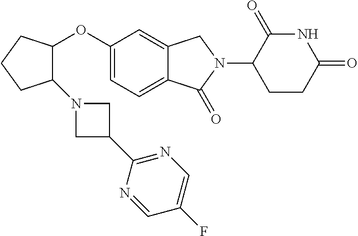 [SMILES: O=C1CCC(N2Cc3cc(OC4CCCC4N4CC(c5ncc(F)cn5)C4)ccc3C2=O)C(=O)N1] | 3-(5-((2-(3-(5-fluoropyrimi- din-2-yl)azetidin-1-yl)cyclo- pentyl)oxy)-1-oxoisoindolin- 2-yl)piperidine-2,6-dione |
| 159-B | 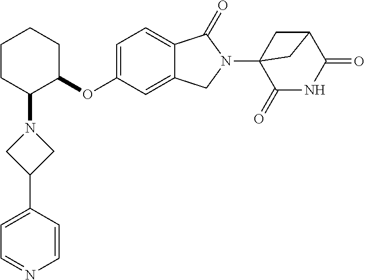 [SMILES: O=C1NC(=O)C2(N3Cc4cc(O[C@@H]5CCCC[C@@H]5N5CC(c6ccncc6)C5)ccc4C3=O)CC1C2] | Rac-1-(1-oxo-5-(((cis)-2-(3- (pyridin-4-yl)azetidin-1-yl)- cyclohexyl)oxy)isoindolin- 2-yl)-3-azabicyclo[3.1.1] heptane-2,4-dione |
| 160-B | 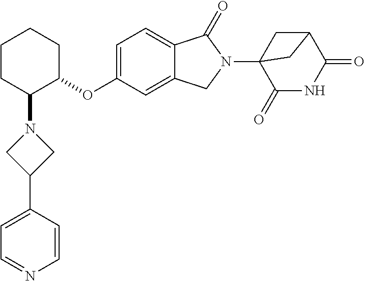 [SMILES: O=C1NC(=O)C2(N3Cc4cc(O[C@H]5CCCC[C@@H]5N5CC(c6ccncc6)C5)ccc4C3=O)CC1C2] | Rac-1-(1-oxo-5-(((trans)-2- (3-(pyridin-4-yl)azetidin-1- yl)cyclohexyl)oxy)isoindolin- 2-yl)-3-azabicyclo[3.1.1] heptane-2,4-dione |
| 161-B | 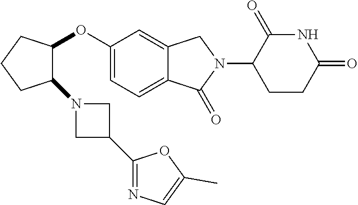 [SMILES: Cc1cnc(C2CN([C@H]3CCC[C@H]3Oc3ccc4c(c3)CN(C3CCC(=O)NC3=O)C4=O)C2)o1] | Rac-3-(5-(((cis)-2-(3-(5- methyloxazol-2-yl)azetidin-1- yl)cyclopentyl)oxy)-1-oxoiso- indolin-2-yl)piperidine-2,6- dione |
| 162-B | 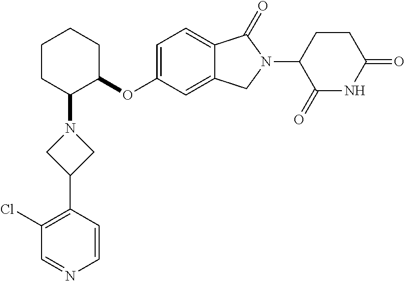 [SMILES: O=C1CCC(N2Cc3cc(O[C@@H]4CCCC[C@@H]4N4CC(c5ccncc5Cl)C4)ccc3C2=O)C(=O)N1] | Rac-3-(5-(((cis)-2-(3-(3- chloropyridin-4-yl)azetidin-1- yl)cyclohexyl)oxy)-1-oxoiso- indolin-2-yl)piperidine-2,6- dione |
| 163-B | 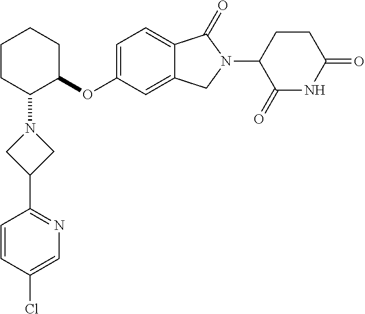 [SMILES: O=C1CCC(N2Cc3cc(O[C@@H]4CCCC[C@H]4N4CC(c5ccc(Cl)cn5)C4)ccc3C2=O)C(=O)N1] | Rac-3-(5-(((trans)-2-(3-(5- chloropyridin-2-yl)azetidin-1- yl)cyclohexyl)oxy)-1-oxoiso- indolin-2-yl)piperidine-2,6- dione |
| 164-B | 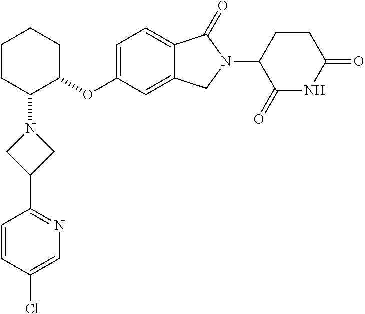 [SMILES: O=C1CCC(N2Cc3cc(O[C@H]4CCCC[C@H]4N4CC(c5ccc(Cl)cn5)C4)ccc3C2=O)C(=O)N1] | Rac-3-(5-(((cis)-2-(3-(5- chloropyridin-2-yl)azetidin-1- yl)cyclohexyl)oxy)-1-oxoiso- indolin-2-yl)piperidine-2,6- dione |
| 165-B | 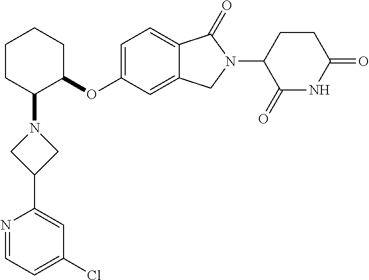 [SMILES: O=C1CCC(N2Cc3cc(O[C@@H]4CCCC[C@@H]4N4CC(c5cc(Cl)ccn5)C4)ccc3C2=O)C(=O)N1] | Rac-3-(5-(((cis)-2-(3-(4- chloropyridin-2-yl)azetidin-1- yl)cyclohexyl)oxy)-1-oxoiso- indolin-2-yl)piperidine-2,6- dione |
| 166-B | 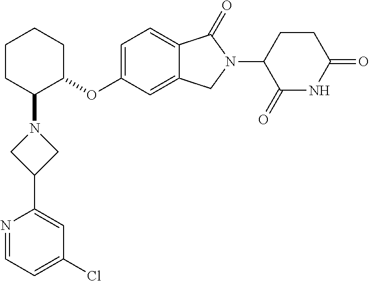 [SMILES: O=C1CCC(N2Cc3cc(O[C@H]4CCCC[C@@H]4N4CC(c5cc(Cl)ccn5)C4)ccc3C2=O)C(=O)N1] | Rac-3-(5-(((trans)-2-(3-(4- chloropyridin-2-yl)azetidin-1- yl)cyclohexyl)oxy)-1-oxoiso- indolin-2-yl)piperidine-2,6- dione |
| 167-B | 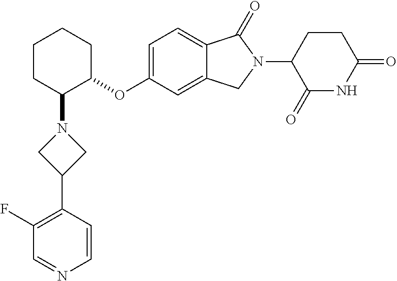 [SMILES: O=C1CCC(N2Cc3cc(O[C@H]4CCCC[C@@H]4N4CC(c5ccncc5F)C4)ccc3C2=O)C(=O)N1] | Rac-3-(5-(((trans)-2-(3-(3- fluoropyridin-4-yl)azetidin-1- yl)cyclohexyl)oxy)-1-oxoiso- indolin-2-yl)piperidine-2,6- dione |
| 168-B | 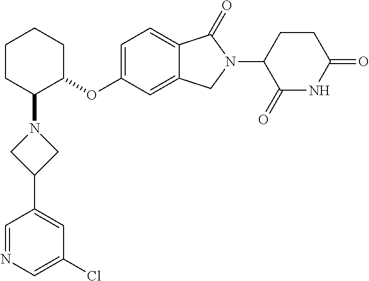 [SMILES: O=C1CCC(N2Cc3cc(O[C@H]4CCCC[C@@H]4N4CC(c5cncc(Cl)c5)C4)ccc3C2=O)C(=O)N1] | Rac-3-(5-(((trans)-2-(3-(5- chloropyridin-3-yl)azetidin-1- yl)cyclohexyl)oxy)-1-oxoiso- indolin-2-yl)piperidine-2,6- dione |
| 169-B | 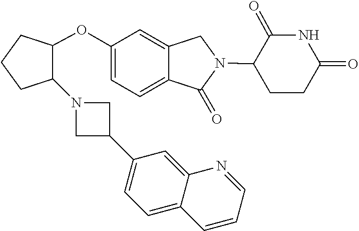 [SMILES: O=C1CCC(N2Cc3cc(OC4CCCC4N4CC(c5ccc6cccnc6c5)C4)ccc3C2=O)C(=O)N1] | 3-(1-oxo-5-((2-(3-(quinolin- 7-yl)azetidin-1-yl)cyclopent- yl)oxy)isoindolin-2-yl)piper- idine-2,6-dione |
| 170-B | 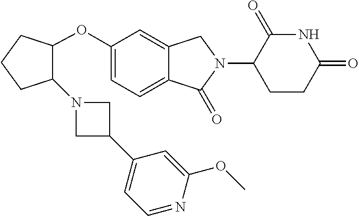 [SMILES: COc1cc(C2CN(C3CCCC3Oc3ccc4c(c3)CN(C3CCC(=O)NC3=O)C4=O)C2)ccn1] | 3-(5-((2-(3-(2-methoxypyridin- 4-yl)azetidin-1-yl)cyclopent- yl)oxy)-1-oxoisoindolin-2- yl)piperidine-2,6-dione |
| 171-B | 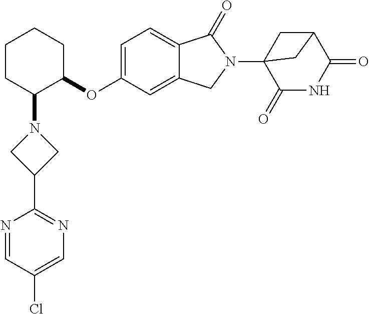 [SMILES: O=C1NC(=O)C2(N3Cc4cc(O[C@@H]5CCCC[C@@H]5N5CC(c6ncc(Cl)cn6)C5)ccc4C3=O)CC1C2] | Rac-1-(5-(((cis)-2-(3-(5-chloro- pyrimidin-2-yl)azetidin-1- yl)cyclohexyl)oxy)-1-oxoiso- indolin-2-yl)-3-azabicyclo [3.1.1]heptane-2,4-dione |
| 172-B | 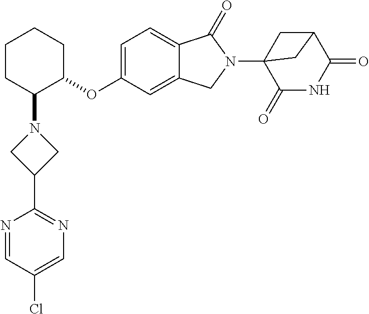 [SMILES: O=C1NC(=O)C2(N3Cc4cc(O[C@H]5CCCC[C@@H]5N5CC(c6ncc(Cl)cn6)C5)ccc4C3=O)CC1C2] | Rac-1-(5-(((trans)-2-(3-(5- chloropyrimidin-2-yl)azetidin- 1-yl)cyclohexyl)oxy)-1-oxo- isoindolin-2-yl)-3-azabicyclo [3.1.1]heptane-2,4-dione |
| 173-B | 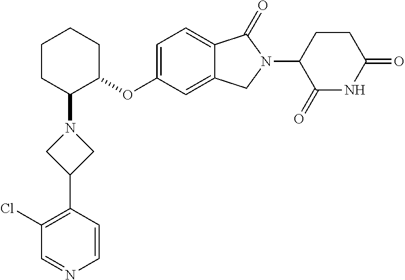 [SMILES: O=C1CCC(N2Cc3cc(O[C@H]4CCCC[C@@H]4N4CC(c5ccncc5Cl)C4)ccc3C2=O)C(=O)N1] | Rac-3-(5-(((trans)-2-(3-(3- chloropyridin-4-yl)azetidin-1- yl)cyclohexyl)oxy)-1-oxoiso- indolin-2-yl)piperidine-2,6- dione |
| 174-B | 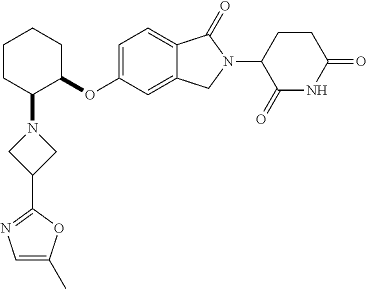 [SMILES: Cc1cnc(C2CN([C@H]3CCCC[C@H]3Oc3ccc4c(c3)CN(C3CCC(=O)NC3=O)C4=O)C2)o1] | Rac-3-(5-(((cis)-2-(3-(5-meth- yloxazol-2-yl)azetidin-1-yl)- cyclohexyl)oxy)-1-oxoiso- indolin-2-yl)piperidine-2,6- dione |
| 175-B | 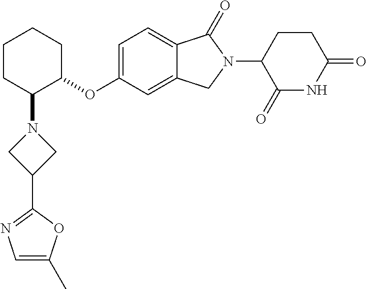 [SMILES: Cc1cnc(C2CN([C@H]3CCCC[C@@H]3Oc3ccc4c(c3)CN(C3CCC(=O)NC3=O)C4=O)C2)o1] | Rac-3-(5-(((trans)-2-(3-(5- methyloxazol-2-yl)azetidin-1- yl)cyclohexyl)oxy)-1-oxoiso- indolin-2-yl)piperidine-2,6- dione |
| 176-B | 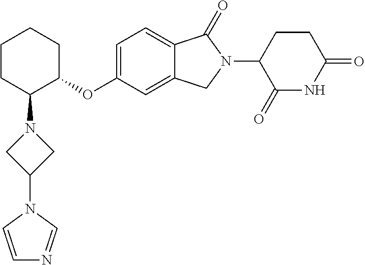 [SMILES: O=C1CCC(N2Cc3cc(O[C@H]4CCCC[C@@H]4N4CC(n5ccnc5)C4)ccc3C2=O)C(=O)N1] | 3-(5-(((1S,2S)-2-(3-(1H- imidazol-1-yl)azetidin-1-yl)- cyclohexyl)oxy)-1-oxoiso- indolin-2-yl)piperidine-2,6- dione |
| 177-B | 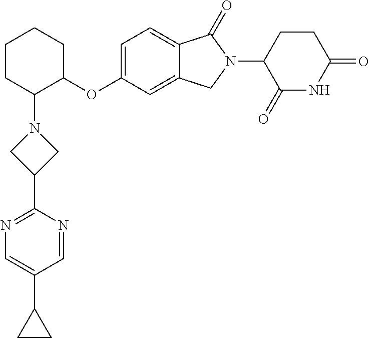 [SMILES: O=C1CCC(N2Cc3cc(OC4CCCCC4N4CC(c5ncc(C6CC6)cn5)C4)ccc3C2=O)C(=O)N1] | 3-(5-((2-(3-(5-cyclopropyl- pyrimidin-2-yl)azetidin-1-yl)- cyclohexyl)oxy)-1-oxoiso- indolin-2-yl)piperidine-2,6- dione |
| 178-B | 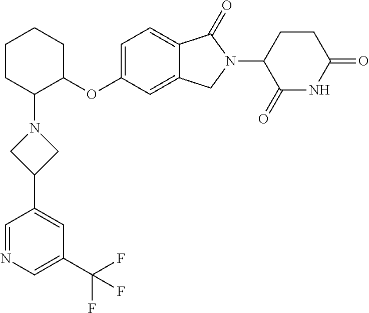 [SMILES: O=C1CCC(N2Cc3cc(OC4CCCCC4N4CC(c5cncc(C(F)(F)F)c5)C4)ccc3C2=O)C(=O)N1] | 3-(1-oxo-5-((2-(3-(5-(trifluoro- methyl)pyridin-3-yl)azetidin- 1-yl)cyclohexyl)oxy)isoin- dolin-2-yl)piperidine-2,6- dione |
| 179-B | 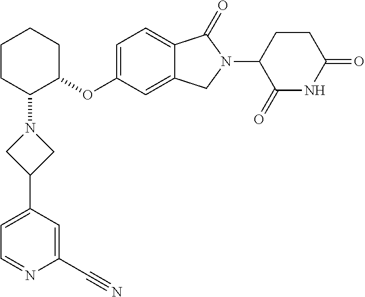 [SMILES: N#Cc1cc(C2CN([C@@H]3CCCC[C@@H]3Oc3ccc4c(c3)CN(C3CCC(=O)NC3=O)C4=O)C2)ccn1] | Rac-4-(1-((cis)-2-((2-(2,6- dioxopiperidin-3-yl)-1-oxo- isoindolin-5-yl)oxy)cyclohex- yl)azetidin-3-yl)picolinonitrile |
| 180-B | 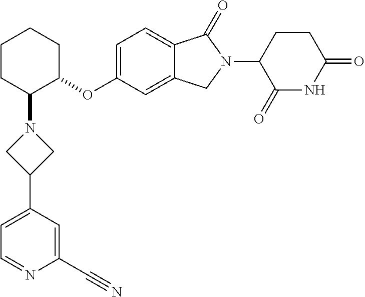 [SMILES: N#Cc1cc(C2CN([C@H]3CCCC[C@@H]3Oc3ccc4c(c3)CN(C3CCC(=O)NC3=O)C4=O)C2)ccn1] | Rac-4-(1-((trans)-2-((2-(2,6- dioxopiperidin-3-yl)-1-oxo- isoindolin-5-yl)oxy)cyclo- hexyl)azetidin-3-yl)picolino- nitrile |
| 181-B | 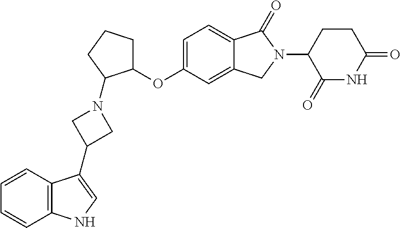 [SMILES: O=C1CCC(N2Cc3cc(OC4CCCC4N4CC(c5c[nH]c6ccccc56)C4)ccc3C2=O)C(=O)N1] | 3-(5-((2-(3-(1H-indol-3-yl)- azetidin-1-yl)cyclopentyl)- oxy)-1-oxoisoindolin-2-yl)- piperidine-2,6-dione |
| 182-B | 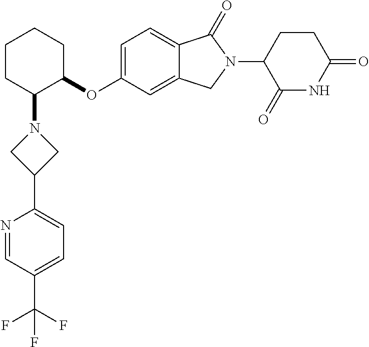 [SMILES: O=C1CCC(N2Cc3cc(O[C@@H]4CCCC[C@@H]4N4CC(c5ccc(C(F)(F)F)cn5)C4)ccc3C2=O)C(=O)N1] | Rac-3-(1-oxo-5-(((cis)-2-(3- (5-(trifluoromethyl)pyridin-2- yl)azetidin-1-yl)cyclohexyl)- oxy)isoindolin-2-yl)piperi- dine-2,6-dione |
| 183-B | 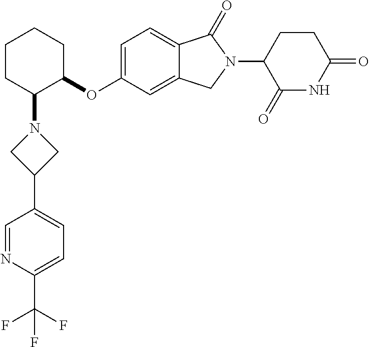 [SMILES: O=C1CCC(N2Cc3cc(O[C@@H]4CCCC[C@@H]4N4CC(c5ccc(C(F)(F)F)nc5)C4)ccc3C2=O)C(=O)N1] | Rac-3-(1-oxo-5-(((cis)-2-(3- (6-(trifluoromethyl)pyridin- 3-yl)azetidin-1-yl)cyclohex- yl)oxy)isoindolin-2-yl)piperi- dine-2,6-dione |
| 184-B | 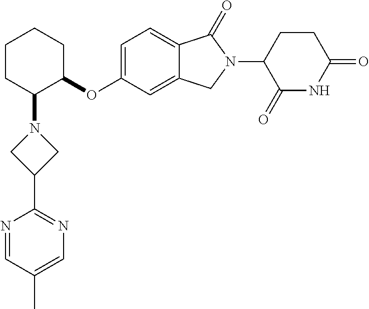 [SMILES: Cc1cnc(C2CN([C@H]3CCCC[C@H]3Oc3ccc4c(c3)CN(C3CCC(=O)NC3=O)C4=O)C2)nc1] | Rac-3-(5-(((cis)-2-(3-(5-meth- ylpyrimidin-2-yl)azetidin-1- yl)cyclohexyl)oxy)-1-oxoiso- indolin-2-yl)piperidine-2,6- dione |
| 185-B | 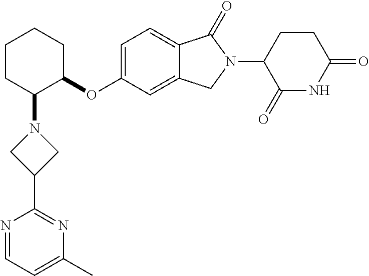 [SMILES: Cc1ccnc(C2CN([C@H]3CCCC[C@H]3Oc3ccc4c(c3)CN(C3CCC(=O)NC3=O)C4=O)C2)n1] | Rac-3-(5-(((cis)-2-(3-(4-meth- ylpyrimidin-2-yl)azetidin-1- yl)cyclohexyl)oxy)-1-oxoiso- indolin-2-yl)piperidine-2,6- dione |
| 186-B | 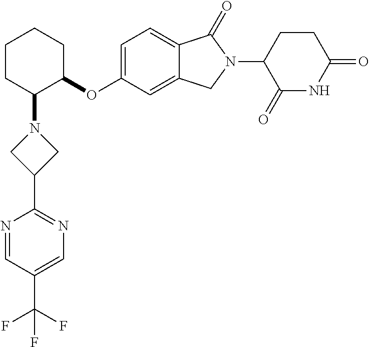 [SMILES: O=C1CCC(N2Cc3cc(O[C@@H]4CCCC[C@@H]4N4CC(c5ncc(C(F)(F)F)cn5)C4)ccc3C2=O)C(=O)N1] | Rac-3-(1-oxo-5-(((cis)-2-(3- (5-(trifluoromethyl)pyrimi- din-2-yl)azetidin-1-yl)cyclo- hexyl)oxy)isoindolin-2-yl)- piperidine-2,6-dione |
| 187-B | 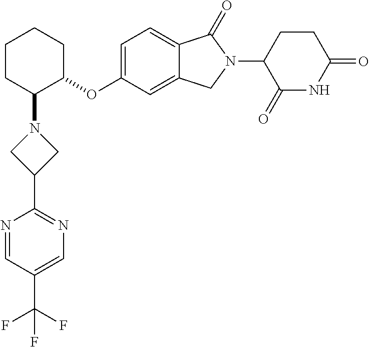 [SMILES: O=C1CCC(N2Cc3cc(O[C@H]4CCCC[C@@H]4N4CC(c5ncc(C(F)(F)F)cn5)C4)ccc3C2=O)C(=O)N1] | Rac-3-(1-oxo-5-(((trans)-2-(3- (5-(trifluoromethyl)pyrimidin- 2-yl)azetidin-1-yl)cyclohex- yl)oxy)isoindolin-2-yl)piperi- dine-2,6-dione |
| 188-B | 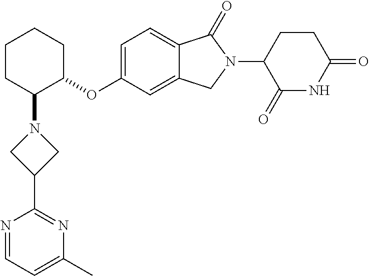 [SMILES: Cc1ccnc(C2CN([C@H]3CCCC[C@@H]3Oc3ccc4c(c3)CN(C3CCC(=O)NC3=O)C4=O)C2)n1] | Rac-3-(5-(((trans)-2-(3-(4- methylpyrimidin-2-yl)azeti- din-1-yl)cyclohexyl)oxy)-1- oxoisoindolin-2-yl)piperidine- 2,6-dione |
| 189-B | 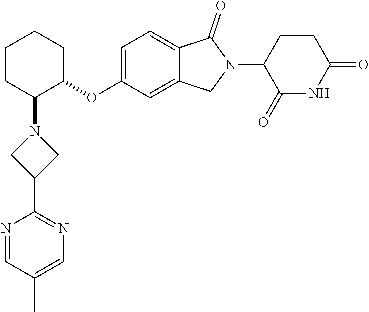 [SMILES: Cc1cnc(C2CN([C@H]3CCCC[C@@H]3Oc3ccc4c(c3)CN(C3CCC(=O)NC3=O)C4=O)C2)nc1] | Rac-3-(5-(((trans)-2-(3-(5- methylpyrimidin-2-yl)azetidin- 1-yl)cyclohexyl)oxy)-1-oxo- isoindolin-2-yl)piperidine-2,6- dione |
| 190-B | 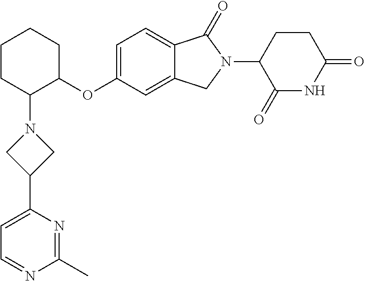 [SMILES: Cc1nccc(C2CN(C3CCCCC3Oc3ccc4c(c3)CN(C3CCC(=O)NC3=O)C4=O)C2)n1] | 3-(5-((2-(3-(2-methylpyrimi- din-4-yl)azetidin-1-yl)cyclo- hexyl)oxy)-1-oxoisoindolin- 2-yl)piperidine-2,6-dione |
| 191-B | 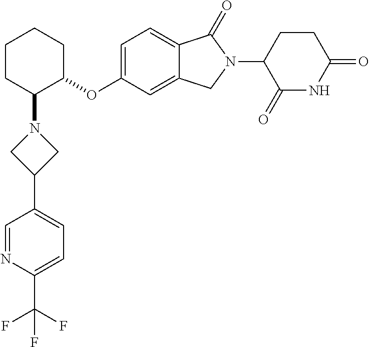 [SMILES: O=C1CCC(N2Cc3cc(O[C@H]4CCCC[C@@H]4N4CC(c5ccc(C(F)(F)F)nc5)C4)ccc3C2=O)C(=O)N1] | Rac-3-(1-oxo-5-(((trans)-2-(3- (6-(trifluoromethyl)pyridin-3- yl)azetidin-1-yl)cyclohexyl)- oxy)isoindolin-2-yl)piperi- dine-2,6-dione |
| 192-B | 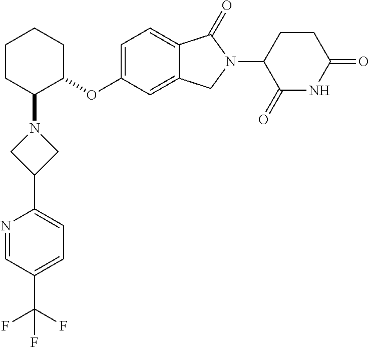 [SMILES: O=C1CCC(N2Cc3cc(O[C@H]4CCCC[C@@H]4N4CC(c5ccc(C(F)(F)F)cn5)C4)ccc3C2=O)C(=O)N1] | Rac-3-(1-oxo-5-(((trans)-2-(3- (5-(trifluoromethyl)pyridin-2- yl)azetidin-1-yl)cyclohexyl)- oxy)isoindolin-2-yl)piperi- dine-2,6-dione |
| 193-B | 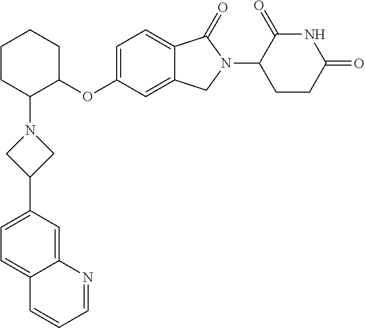 [SMILES: O=C1CCC(N2Cc3cc(OC4CCCCC4N4CC(c5ccc6cccnc6c5)C4)ccc3C2=O)C(=O)N1] | 3-(1-oxo-5-((2-(3-(quinolin-7- yl)azetidin-1-yl)cyclohexyl)- oxy)isoindolin-2-yl)piperi- dine-2,6-dione |
| 194-B | 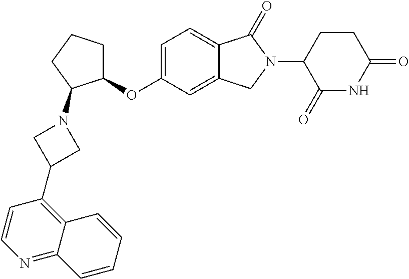 [SMILES: O=C1CCC(N2Cc3cc(O[C@@H]4CCC[C@@H]4N4CC(c5ccnc6ccccc56)C4)ccc3C2=O)C(=O)N1] | 3-(1-oxo-5-(((1R,2S)-2-(3- (quinolin-4-yl)azetidin-1-yl)- cyclopentyl)oxy)isoindolin-2- yl)piperidine-2,6-dione |
| 195-B | 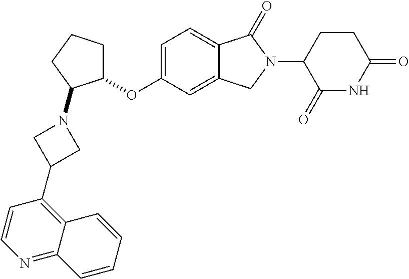 [SMILES: O=C1CCC(N2Cc3cc(O[C@H]4CCC[C@@H]4N4CC(c5ccnc6ccccc56)C4)ccc3C2=O)C(=O)N1] | Rac-3-(1-oxo-5-(((trans)-2- (3-(quinolin-4-yl)azetidin-1- yl)cyclopentyl)oxy)isoindolin- 2-yl)piperidine-2,6-dione |
| 196-B | 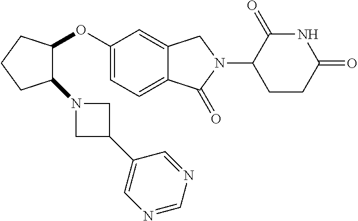 | Rac-3-(1-oxo-5-(((cis)-2-(3- (pyrimidin-5-yl)azetidin-1- yl)cyclopentyl)oxy)isoindolin- 2-yl)piperidine-2,6-dione |
| 197-B | 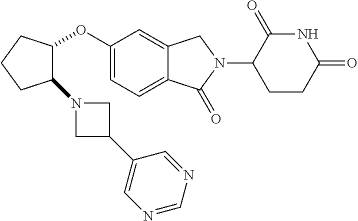 | Rac-3-(1-oxo-5-(((trans)-2-(3- (pyrimidin-5-yl)azetidin-1- yl)cyclopentyl)oxy)isoindolin- 2-yl)piperidine-2,6-dione |
| 198-B | 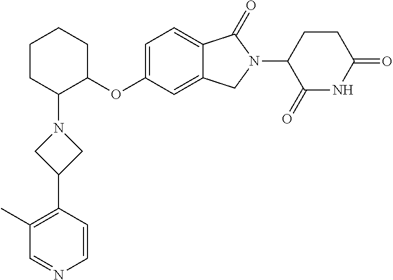 [SMILES: Cc1cnccc1C1CN(C2CCCCC2Oc2ccc3c(c2)CN(C2CCC(=O)NC2=O)C3=O)C1] | 3-(5-((2-(3-(3-methylpyridin- 4-yl)azetidin-1-yl)cyclohex- yl)oxy)-1-oxoisoindolin-2-yl)- piperidine-2,6-dione |
| 199-B | 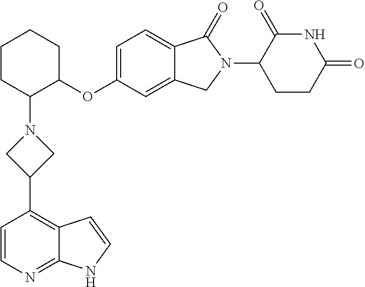 [SMILES: O=C1CCC(N2Cc3cc(OC4CCCCC4N4CC(c5ccnc6[nH]ccc56)C4)ccc3C2=O)C(=O)N1] | 3-(5-((2-(3-(1H-pyrrolo[2,3- b]pyridin-4-yl)azetidin-1-yl)- cyclohexyl)oxy)-1-oxoiso- indolin-2-yl)piperidine-2,6- dione |
| 200-B | 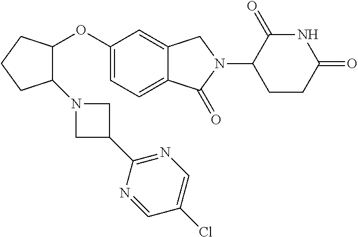 [SMILES: O=C1CCC(N2Cc3cc(OC4CCCC4N4CC(c5ncc(Cl)cn5)C4)ccc3C2=O)C(=O)N1] | 3-(5-((2-(3-(5-chloropyrimi- din-2-yl)azetidin-1-yl)cyclo- pentyl)oxy)-1-oxoisoindolin- 2-yl)piperidine-2,6-dione |
| 201-B | 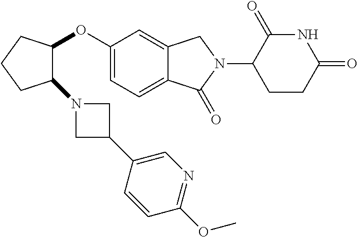 | Rac-3-(5-(((cis)-2-(3-(6-meth- oxypyridin-3-yl)azetidin-1- yl)cyclopentyl)oxy)-1-oxo- isoindolin-2-yl)piperidine- 2,6-dione |
| 202-B | 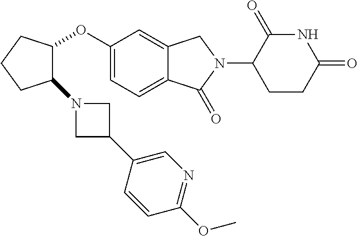 | Rac-3-(5-(((trans)-2-(3-(6- methoxypyridin-3-yl)azetidin- 1-yl)cyclopentyl)oxy)-1-oxo- isoindolin-2-yl)piperidine- 2,6-dione |
| 203-B | 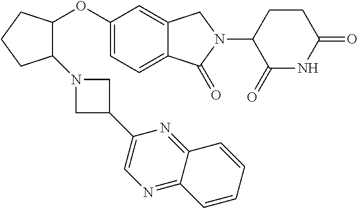 [SMILES: O=C1CCC(N2Cc3cc(OC4CCCC4N4CC(c5cnc6ccccc6n5)C4)ccc3C2=O)C(=O)N1] | 3-(1-oxo-5-((2-(3-(quinoxalin- 2-yl)azetidin-1-yl)cyclopent- yl)oxy)isoindolin-2-yl)piperi- dine-2,6-dione |
| 204-B | 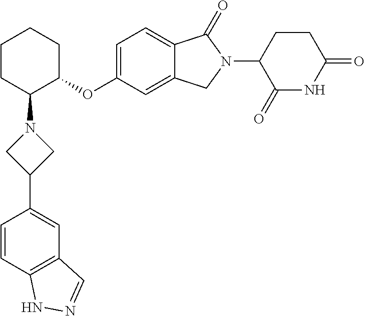 [SMILES: O=C1CCC(N2Cc3cc(O[C@H]4CCCC[C@@H]4N4CC(c5ccc6[nH]ncc6c5)C4)ccc3C2=O)C(=O)N1] | 3-(5-(((1S,2S)-2-(3-(1H- indazol-5-yl)azetidin-1-yl)- cyclohexyl)oxy)-1-oxoiso- indolin-2-yl)piperidine-2,6- dione |
| 205-B | 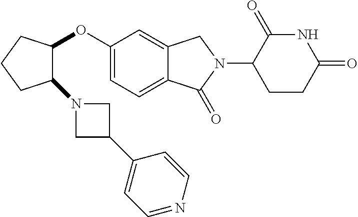 [SMILES: O=C1CCC(N2Cc3cc(O[C@@H]4CCC[C@@H]4N4CC(c5ccncc5)C4)ccc3C2=O)C(=O)N1] | Rac-3-(1-oxo-5-(((cis)-2-(3- (pyridin-4-yl)azetidin-1-yl)- cyclopentyl)oxy)isoindolin-2- yl)piperidine-2,6-dione |
| 206-B | 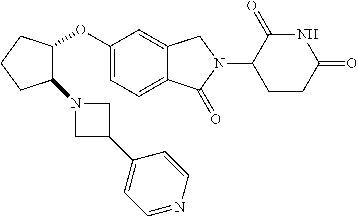 [SMILES: O=C1CCC(N2Cc3cc(O[C@H]4CCC[C@@H]4N4CC(c5ccncc5)C4)ccc3C2=O)C(=O)N1] | Rac-3-(1-oxo-5-(((trans)-2-(3- (pyridin-4-yl)azetidin-1-yl)- cyclopentyl)oxy)isoindolin-2- yl)piperidine-2,6-dione |
| 207-B | 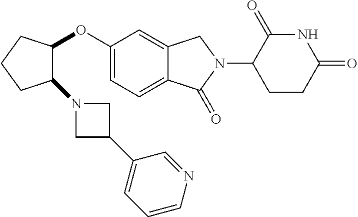 [SMILES: O=C1CCC(N2Cc3cc(O[C@@H]4CCC[C@@H]4N4CC(c5cccnc5)C4)ccc3C2=O)C(=O)N1] | Rac-3-(1-oxo-5-(((cis)-2-(3- (pyridin-3-yl)azetidin-1-yl)- cyclopentyl)oxy)isoindolin-2- yl)piperidine-2,6-dione |
| 208-B | 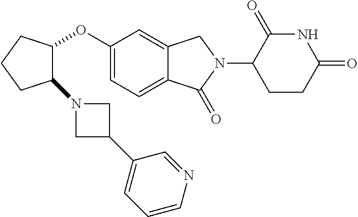 [SMILES: O=C1CCC(N2Cc3cc(O[C@H]4CCC[C@@H]4N4CC(c5cccnc5)C4)ccc3C2=O)C(=O)N1] | Rac-3-(1-oxo-5-(((trans)-2-(3- (pyridin-3-yl)azetidin-1-yl)- cyclopentyl)oxy)isoindolin-2- yl)piperidine-2,6-dione |
| 209-B | 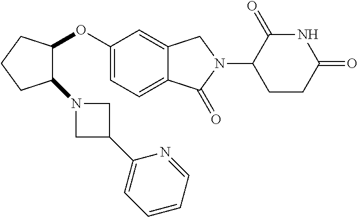 [SMILES: O=C1CCC(N2Cc3cc(O[C@@H]4CCC[C@@H]4N4CC(c5ccccn5)C4)ccc3C2=O)C(=O)N1] | Rac-3-(1-oxo-5-(((cis)-2-(3- (pyridin-2-yl)azetidin-1-yl)- cyclopentyl)oxy)isoindolin-2- yl)piperidine-2,6-dione |
| 210-B | 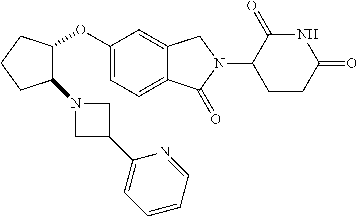 [SMILES: O=C1CCC(N2Cc3cc(O[C@H]4CCC[C@@H]4N4CC(c5ccccn5)C4)ccc3C2=O)C(=O)N1] | Rac-3-(1-oxo-5-(((trans)-2-(3- (pyridin-2-yl)azetidin-1-yl)- cyclopentyl)oxy)isoindolin-2- yl)piperidine-2,6-dione |
| 211-B | 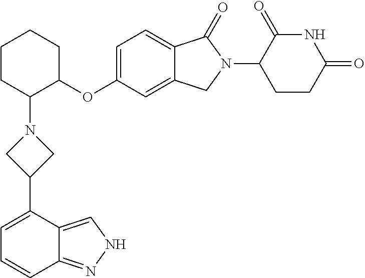 [SMILES: O=C1CCC(N2Cc3cc(OC4CCCCC4N4CC(c5cccc6n[nH]cc56)C4)ccc3C2=O)C(=O)N1] | 3-(5-((2-(3-(2H-indazol-4-yl)- azetidin-1-yl)cyclohexyl)oxy)- 1-oxoisoindolin-2-yl)piperi- dine-2,6-dione |
| 212-B | 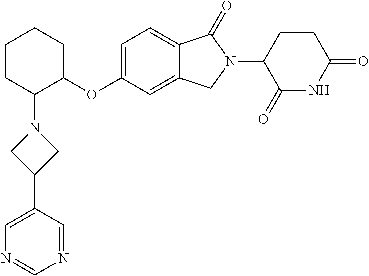 [SMILES: O=C1CCC(N2Cc3cc(OC4CCCCC4N4CC(c5cncnc5)C4)ccc3C2=O)C(=O)N1] | 3-(1-oxo-5-((2-(3-(pyrimidin- 5-yl)azetidin-1-yl)cyclohex- yl)-oxy)isoindolin-2-yl)piperi- dine-2,6-dione |

or wherein the compound is selected from:
Structure 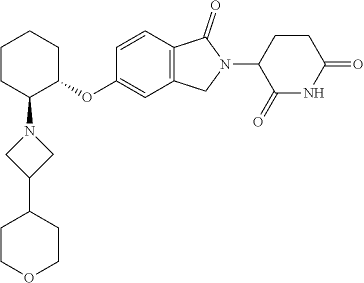 [SMILES: O=C1CCC(N2Cc3cc(O[C@H]4CCCC[C@@H]4N4CC(C5CCOCC5)C4)ccc3C2=O)C(=O)N1] 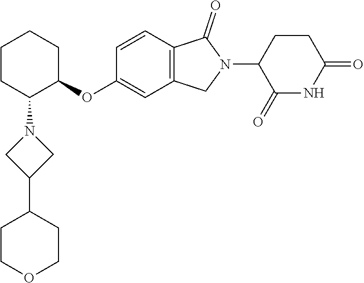 [SMILES: O=C1CCC(N2Cc3cc(O[C@@H]4CCCC[C@H]4N4CC(C5CCOCC5)C4)ccc3C2=O)C(=O)N1] 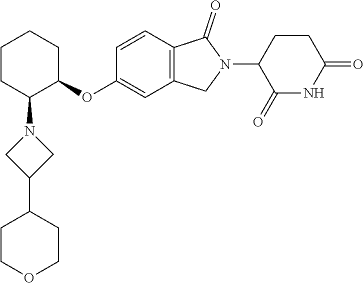 [SMILES: O=C1CCC(N2Cc3cc(O[C@@H]4CCCC[C@@H]4N4CC(C5CCOCC5)C4)ccc3C2=O)C(=O)N1] 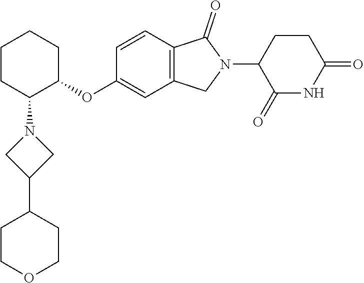 [SMILES: O=C1CCC(N2Cc3cc(O[C@H]4CCCC[C@H]4N4CC(C5CCOCC5)C4)ccc3C2=O)C(=O)N1] 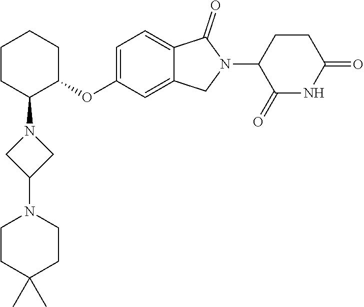 [SMILES: CC1(C)CCN(C2CN([C@H]3CCCC[C@@H]3Oc3ccc4c(c3)CN(C3CCC(=O)NC3=O)C4=O)C2)CC1] 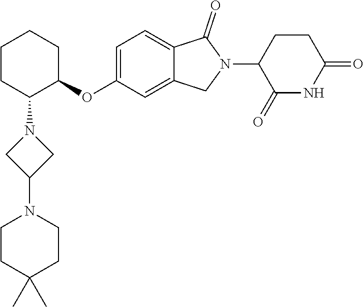 [SMILES: CC1(C)CCN(C2CN([C@@H]3CCCC[C@H]3Oc3ccc4c(c3)CN(C3CCC(=O)NC3=O)C4=O)C2)CC1] 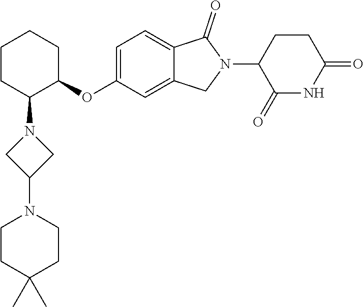 [SMILES: CC1(C)CCN(C2CN([C@H]3CCCC[C@H]3Oc3ccc4c(c3)CN(C3CCC(=O)NC3=O)C4=O)C2)CC1] 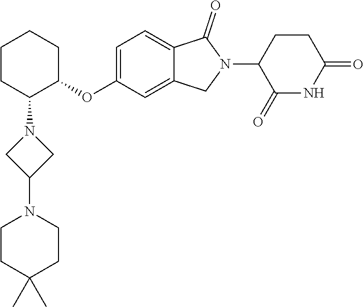 [SMILES: CC1(C)CCN(C2CN([C@@H]3CCCC[C@@H]3Oc3ccc4c(c3)CN(C3CCC(=O)NC3=O)C4=O)C2)CC1] 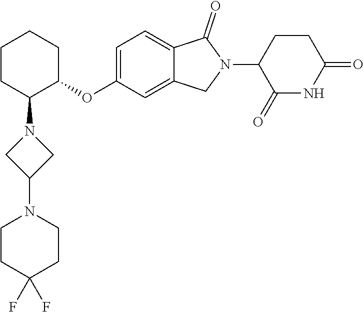 [SMILES: O=C1CCC(N2Cc3cc(O[C@H]4CCCC[C@@H]4N4CC(N5CCC(F)(F)CC5)C4)ccc3C2=O)C(=O)N1] 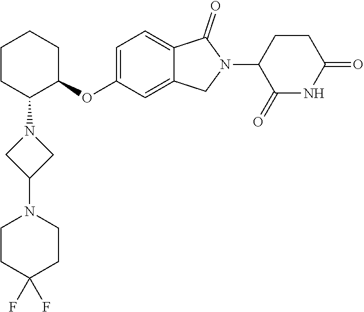 [SMILES: O=C1CCC(N2Cc3cc(O[C@@H]4CCCC[C@H]4N4CC(N5CCC(F)(F)CC5)C4)ccc3C2=O)C(=O)N1] 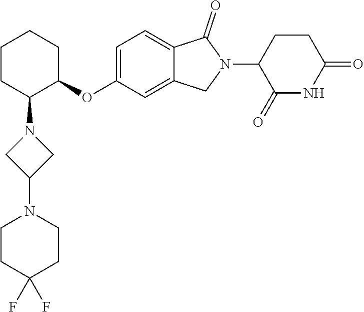 [SMILES: O=C1CCC(N2Cc3cc(O[C@@H]4CCCC[C@@H]4N4CC(N5CCC(F)(F)CC5)C4)ccc3C2=O)C(=O)N1] 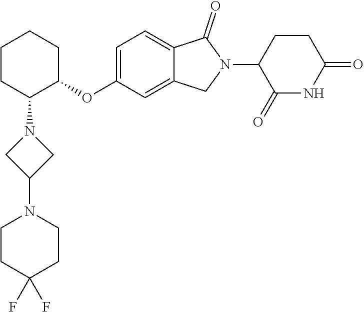 [SMILES: O=C1CCC(N2Cc3cc(O[C@H]4CCCC[C@H]4N4CC(N5CCC(F)(F)CC5)C4)ccc3C2=O)C(=O)N1] 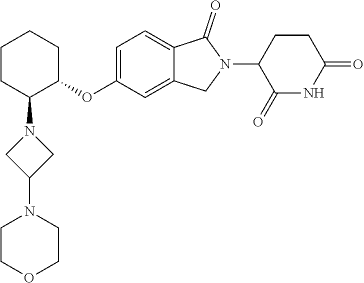 [SMILES: O=C1CCC(N2Cc3cc(O[C@H]4CCCC[C@@H]4N4CC(N5CCOCC5)C4)ccc3C2=O)C(=O)N1] 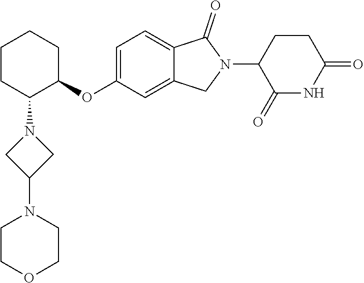 [SMILES: O=C1CCC(N2Cc3cc(O[C@@H]4CCCC[C@H]4N4CC(N5CCOCC5)C4)ccc3C2=O)C(=O)N1] 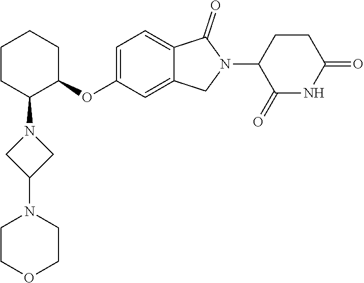 [SMILES: O=C1CCC(N2Cc3cc(O[C@@H]4CCCC[C@@H]4N4CC(N5CCOCC5)C4)ccc3C2=O)C(=O)N1] 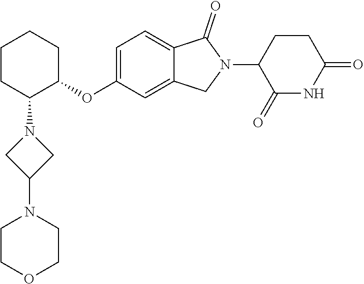 [SMILES: O=C1CCC(N2Cc3cc(O[C@H]4CCCC[C@H]4N4CC(N5CCOCC5)C4)ccc3C2=O)C(=O)N1] 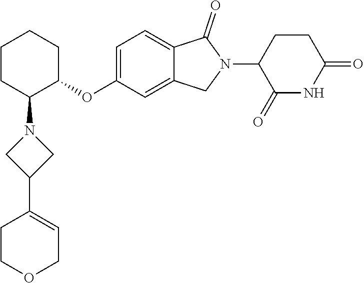 [SMILES: O=C1CCC(N2Cc3cc(O[C@H]4CCCC[C@@H]4N4CC(C5=CCOCC5)C4)ccc3C2=O)C(=O)N1] 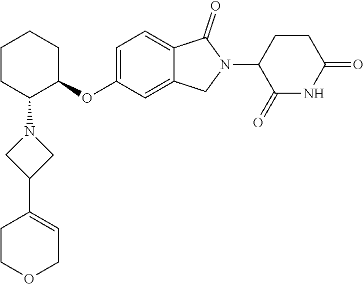 [SMILES: O=C1CCC(N2Cc3cc(O[C@@H]4CCCC[C@H]4N4CC(C5=CCOCC5)C4)ccc3C2=O)C(=O)N1] 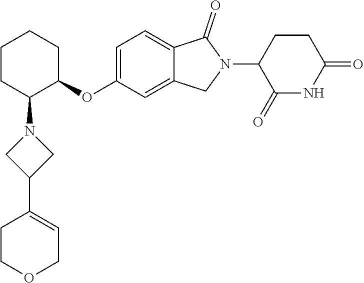 [SMILES: O=C1CCC(N2Cc3cc(O[C@@H]4CCCC[C@@H]4N4CC(C5=CCOCC5)C4)ccc3C2=O)C(=O)N1] 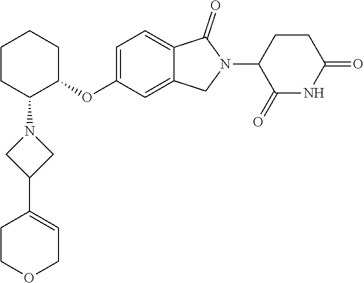 [SMILES: O=C1CCC(N2Cc3cc(O[C@H]4CCCC[C@H]4N4CC(C5=CCOCC5)C4)ccc3C2=O)C(=O)N1] 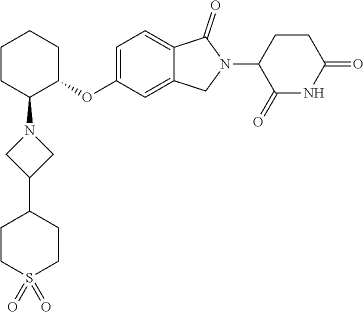 [SMILES: O=C1CCC(N2Cc3cc(O[C@H]4CCCC[C@@H]4N4CC(C5CCS(=O)(=O)CC5)C4)ccc3C2=O)C(=O)N1] 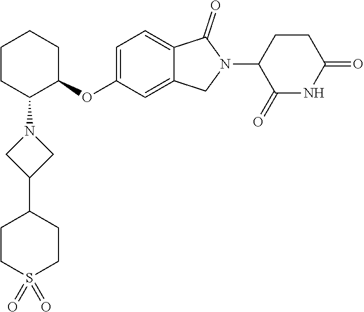 [SMILES: O=C1CCC(N2Cc3cc(O[C@@H]4CCCC[C@H]4N4CC(C5CCS(=O)(=O)CC5)C4)ccc3C2=O)C(=O)N1] 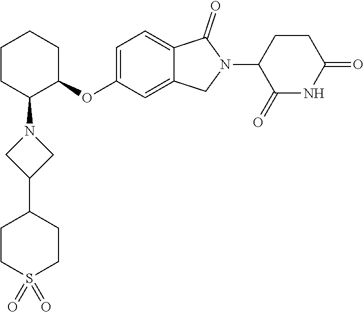 [SMILES: O=C1CCC(N2Cc3cc(O[C@@H]4CCCC[C@@H]4N4CC(C5CCS(=O)(=O)CC5)C4)ccc3C2=O)C(=O)N1] 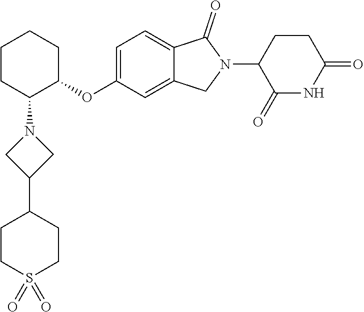 [SMILES: O=C1CCC(N2Cc3cc(O[C@H]4CCCC[C@H]4N4CC(C5CCS(=O)(=O)CC5)C4)ccc3C2=O)C(=O)N1] 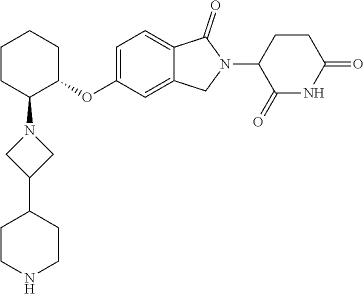 [SMILES: O=C1CCC(N2Cc3cc(O[C@H]4CCCC[C@@H]4N4CC(C5CCNCC5)C4)ccc3C2=O)C(=O)N1] 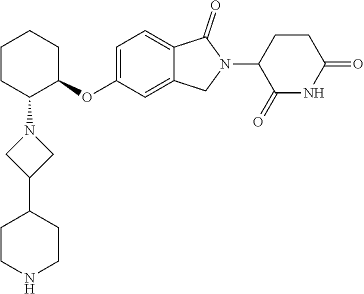 [SMILES: O=C1CCC(N2Cc3cc(O[C@@H]4CCCC[C@H]4N4CC(C5CCNCC5)C4)ccc3C2=O)C(=O)N1] 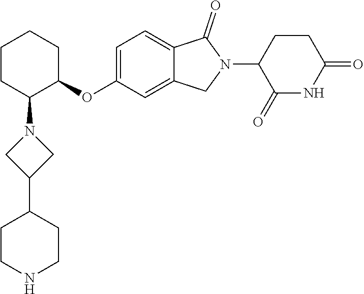 [SMILES: O=C1CCC(N2Cc3cc(O[C@@H]4CCCC[C@@H]4N4CC(C5CCNCC5)C4)ccc3C2=O)C(=O)N1] 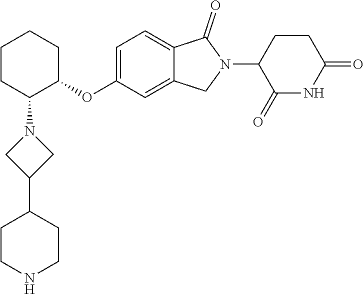 [SMILES: O=C1CCC(N2Cc3cc(O[C@H]4CCCC[C@H]4N4CC(C5CCNCC5)C4)ccc3C2=O)C(=O)N1] 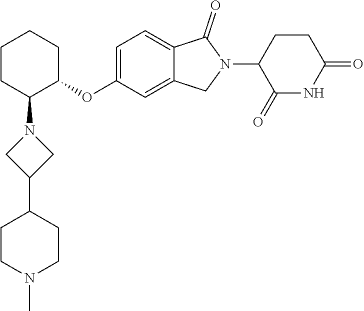 [SMILES: CN1CCC(C2CN([C@H]3CCCC[C@@H]3Oc3ccc4c(c3)CN(C3CCC(=O)NC3=O)C4=O)C2)CC1] 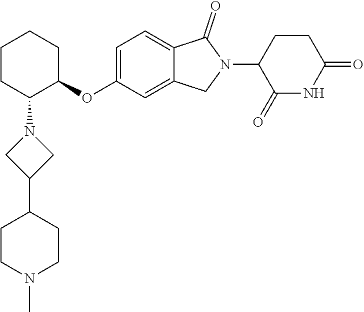 [SMILES: CN1CCC(C2CN([C@@H]3CCCC[C@H]3Oc3ccc4c(c3)CN(C3CCC(=O)NC3=O)C4=O)C2)CC1] 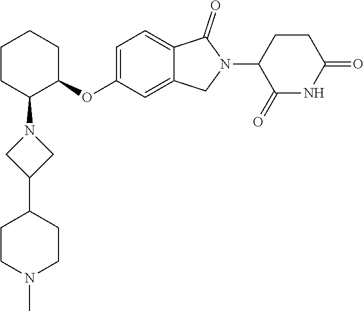 [SMILES: CN1CCC(C2CN([C@H]3CCCC[C@H]3Oc3ccc4c(c3)CN(C3CCC(=O)NC3=O)C4=O)C2)CC1] 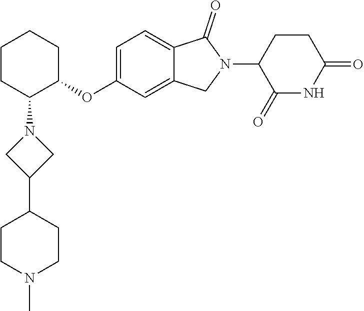 [SMILES: CN1CCC(C2CN([C@@H]3CCCC[C@@H]3Oc3ccc4c(c3)CN(C3CCC(=O)NC3=O)C4=O)C2)CC1] 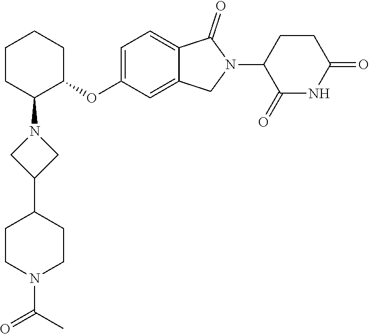 [SMILES: CC(=O)N1CCC(C2CN([C@H]3CCCC[C@@H]3Oc3ccc4c(c3)CN(C3CCC(=O)NC3=O)C4=O)C2)CC1] 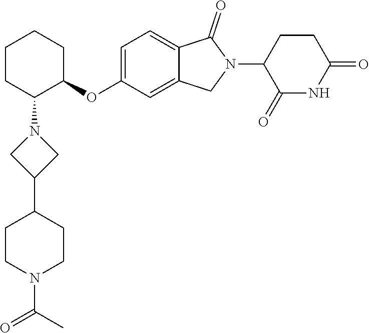 [SMILES: CC(=O)N1CCC(C2CN([C@@H]3CCCC[C@H]3Oc3ccc4c(c3)CN(C3CCC(=O)NC3=O)C4=O)C2)CC1] 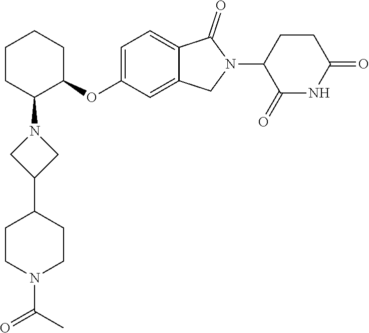 [SMILES: CC(=O)N1CCC(C2CN([C@H]3CCCC[C@H]3Oc3ccc4c(c3)CN(C3CCC(=O)NC3=O)C4=O)C2)CC1] 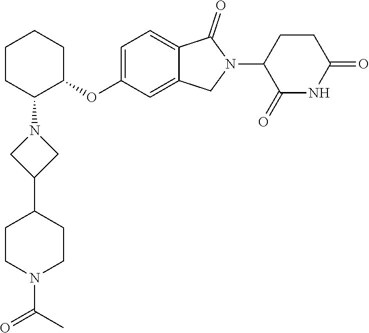 [SMILES: CC(=O)N1CCC(C2CN([C@@H]3CCCC[C@@H]3Oc3ccc4c(c3)CN(C3CCC(=O)NC3=O)C4=O)C2)CC1] 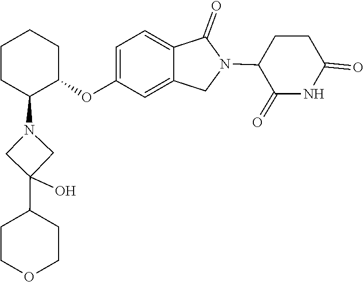 [SMILES: O=C1CCC(N2Cc3cc(O[C@H]4CCCC[C@@H]4N4CC(O)(C5CCOCC5)C4)ccc3C2=O)C(=O)N1] 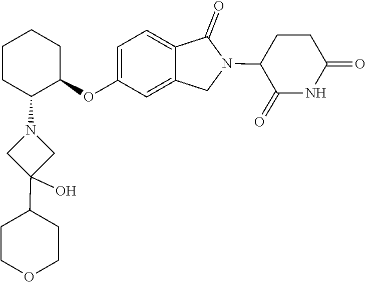 [SMILES: O=C1CCC(N2Cc3cc(O[C@@H]4CCCC[C@H]4N4CC(O)(C5CCOCC5)C4)ccc3C2=O)C(=O)N1] 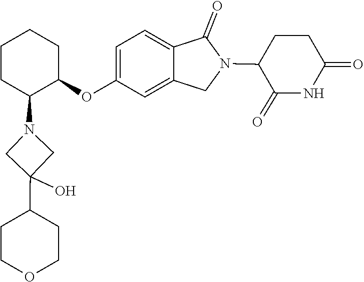 [SMILES: O=C1CCC(N2Cc3cc(O[C@@H]4CCCC[C@@H]4N4CC(O)(C5CCOCC5)C4)ccc3C2=O)C(=O)N1] 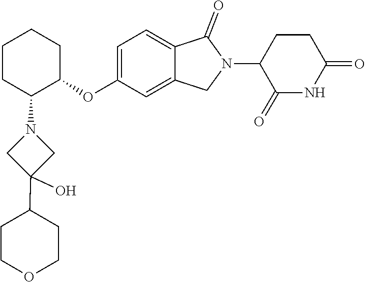 [SMILES: O=C1CCC(N2Cc3cc(O[C@H]4CCCC[C@H]4N4CC(O)(C5CCOCC5)C4)ccc3C2=O)C(=O)N1] 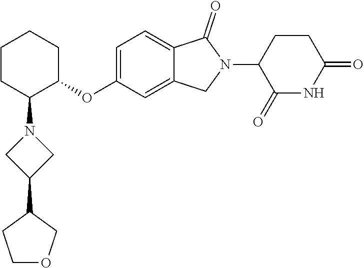 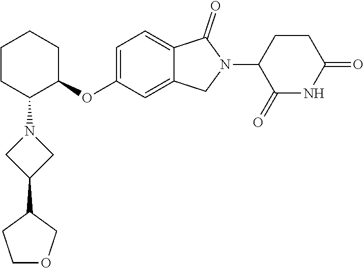 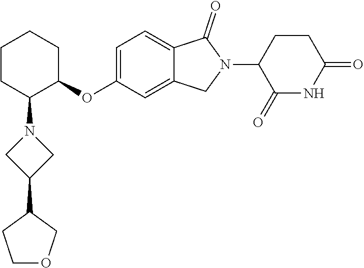 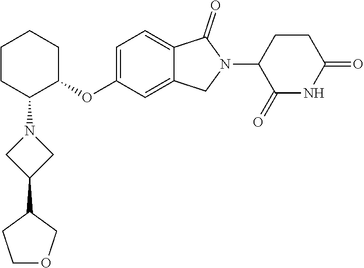 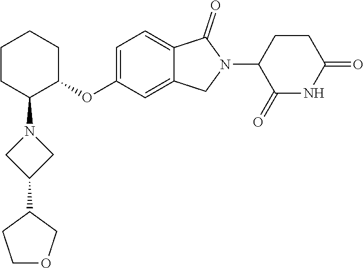 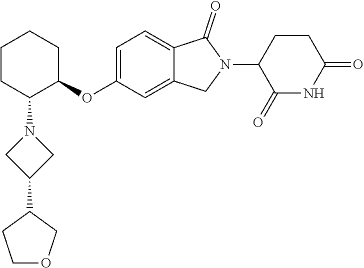 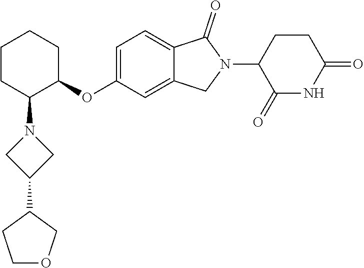 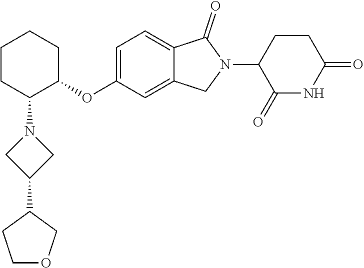 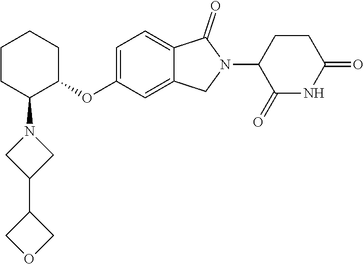 [SMILES: O=C1CCC(N2Cc3cc(O[C@H]4CCCC[C@@H]4N4CC(C5COC5)C4)ccc3C2=O)C(=O)N1] 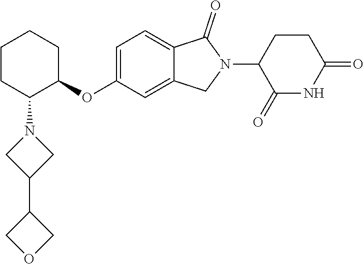 [SMILES: O=C1CCC(N2Cc3cc(O[C@@H]4CCCC[C@H]4N4CC(C5COC5)C4)ccc3C2=O)C(=O)N1] 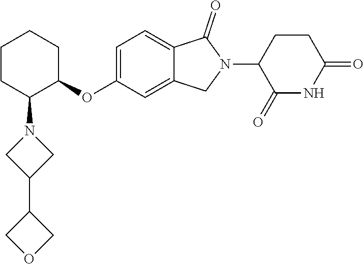 [SMILES: O=C1CCC(N2Cc3cc(O[C@@H]4CCCC[C@@H]4N4CC(C5COC5)C4)ccc3C2=O)C(=O)N1] 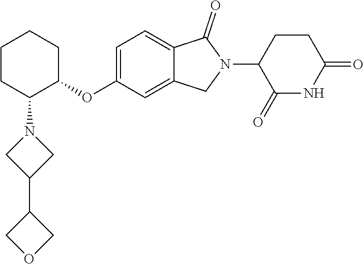 [SMILES: O=C1CCC(N2Cc3cc(O[C@H]4CCCC[C@H]4N4CC(C5COC5)C4)ccc3C2=O)C(=O)N1] 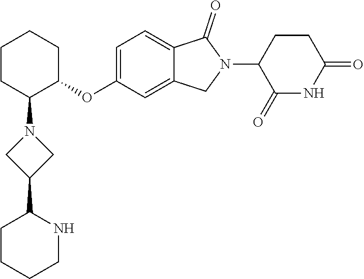 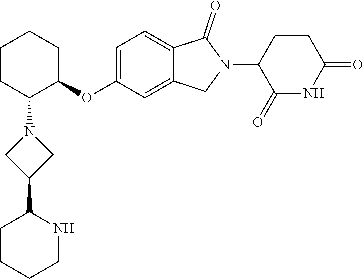 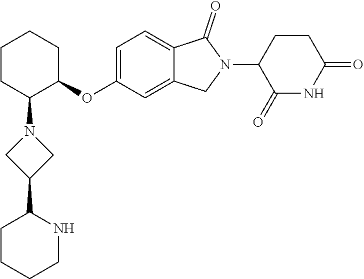 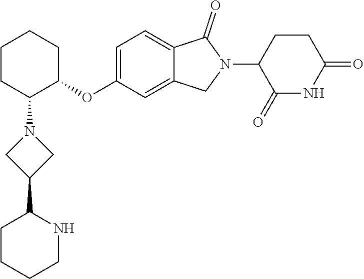 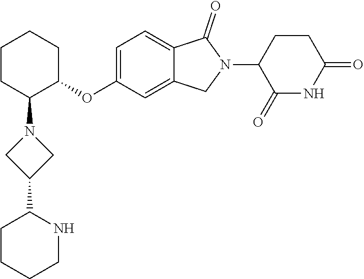 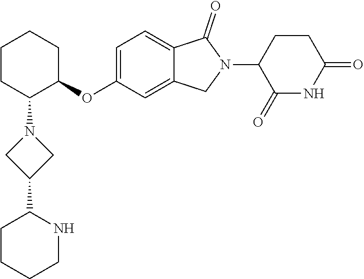 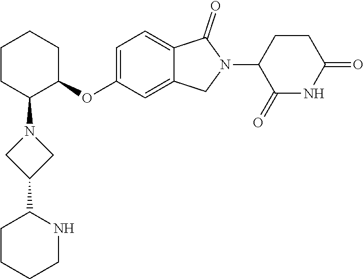 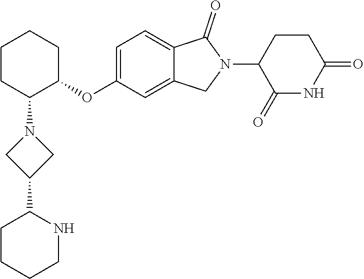 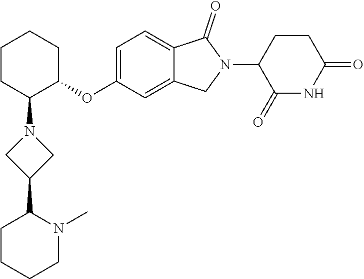 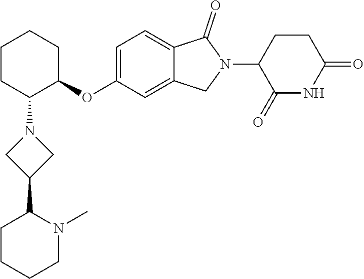 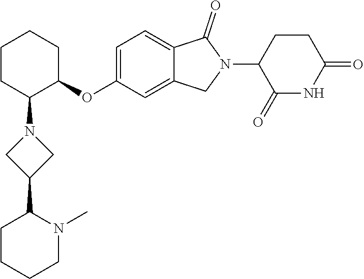 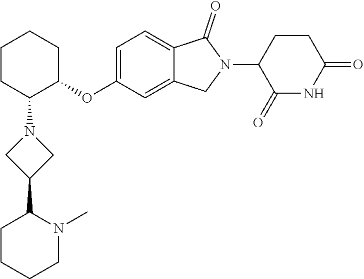 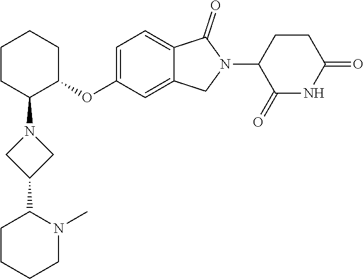 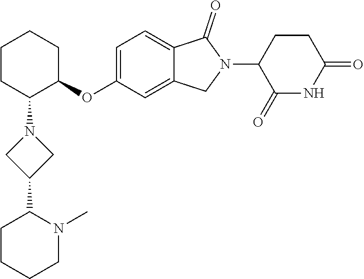 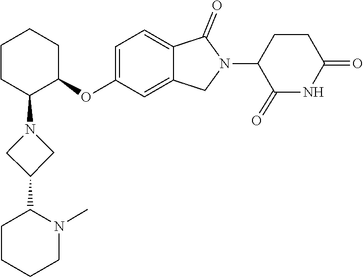 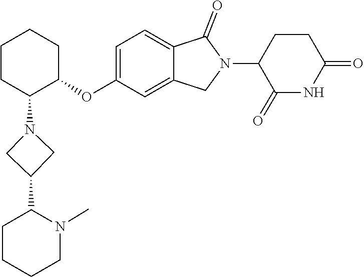 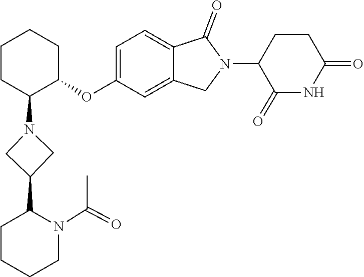 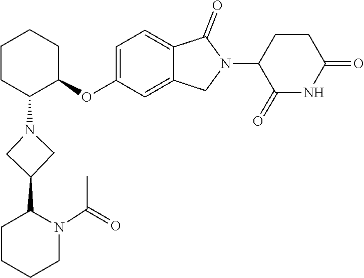 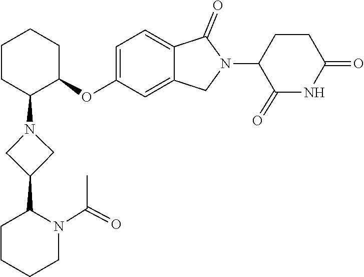 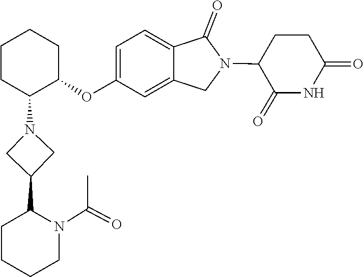 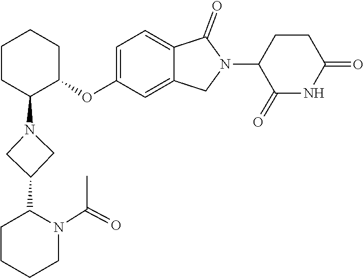 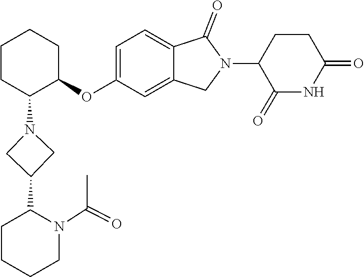 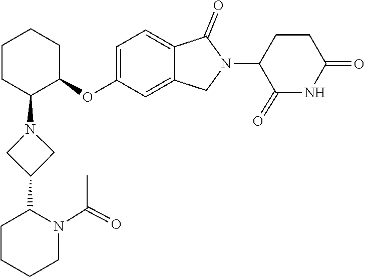 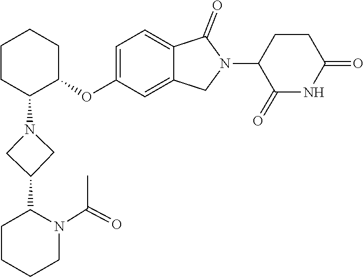 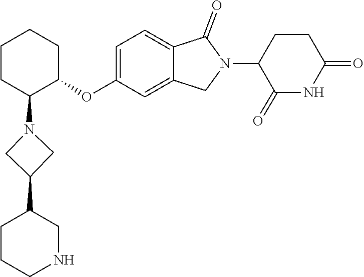 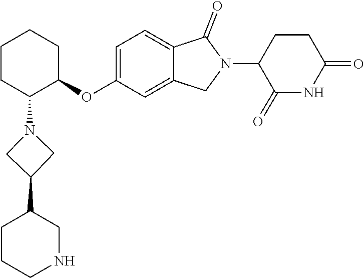 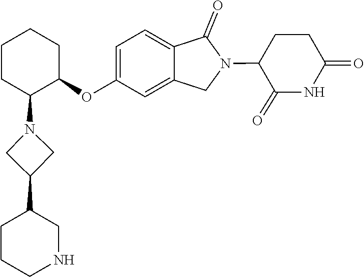 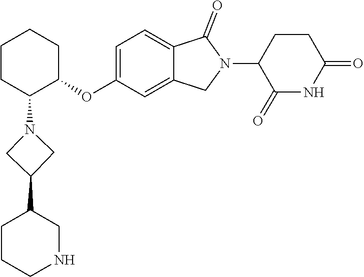 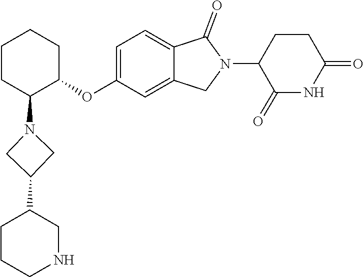 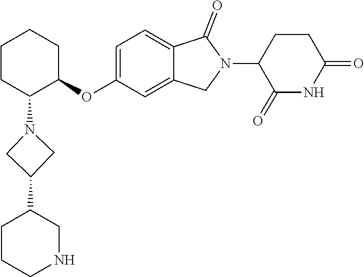 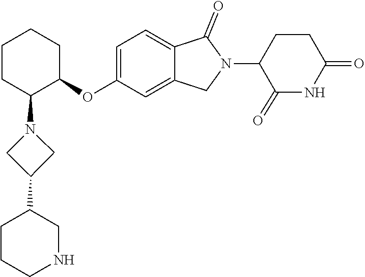 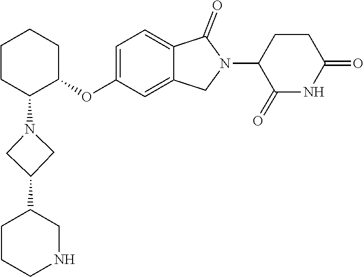 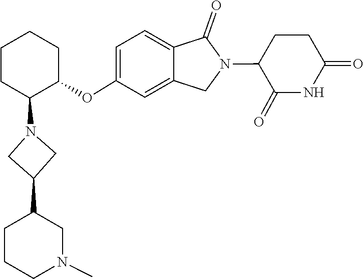 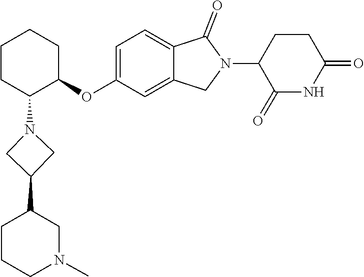 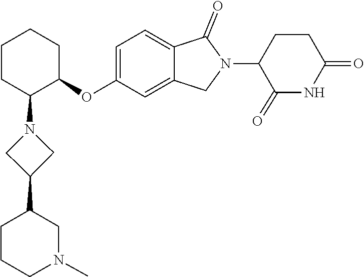 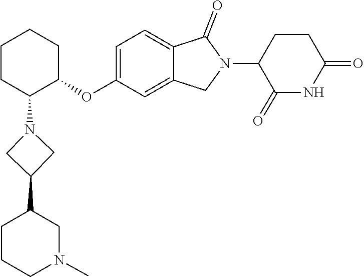 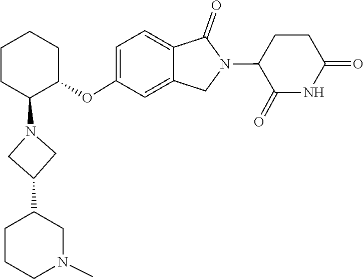 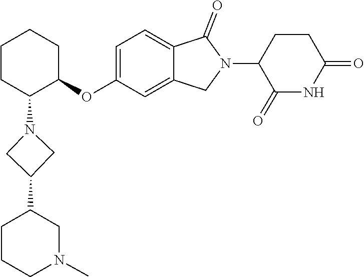 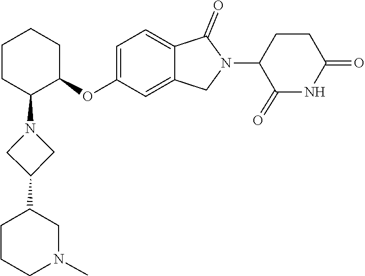 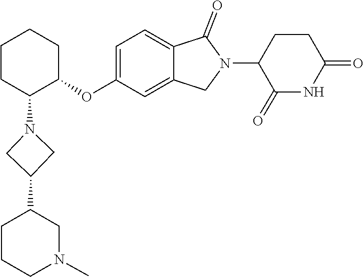 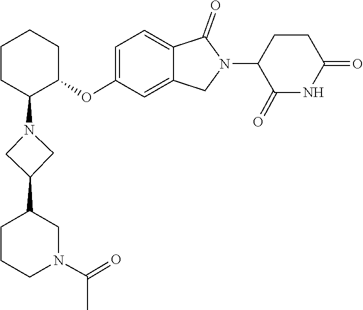 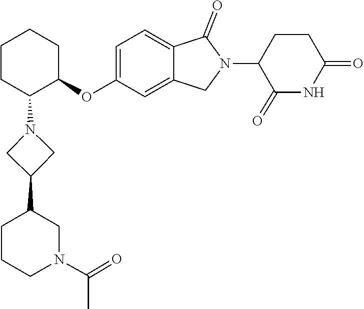 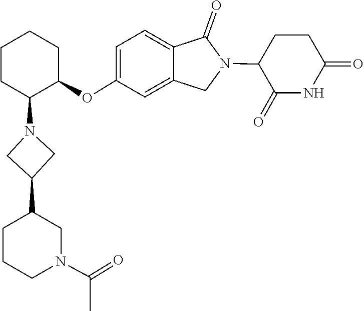 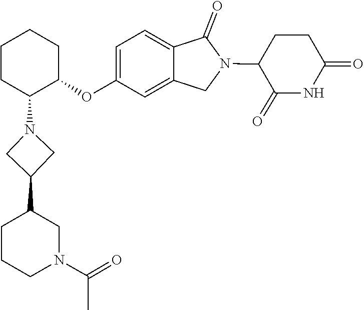 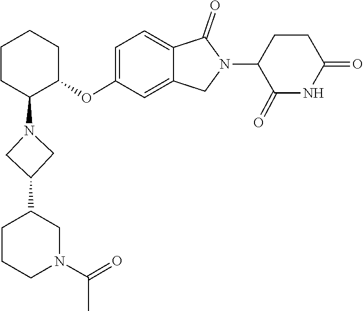 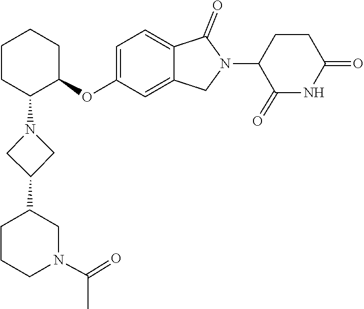 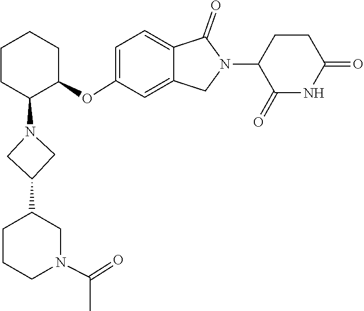 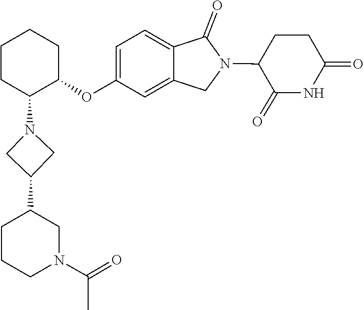 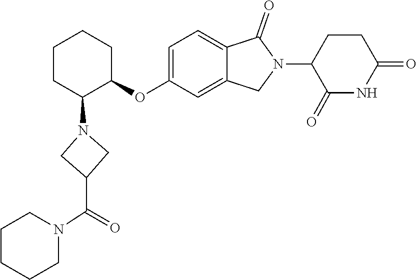 [SMILES: O=C1CCC(N2Cc3cc(O[C@@H]4CCCC[C@@H]4N4CC(C(=O)N5CCCCC5)C4)ccc3C2=O)C(=O)N1] 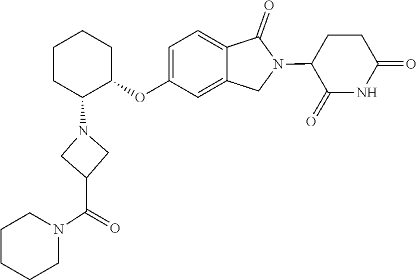 [SMILES: O=C1CCC(N2Cc3cc(O[C@H]4CCCC[C@H]4N4CC(C(=O)N5CCCCC5)C4)ccc3C2=O)C(=O)N1] 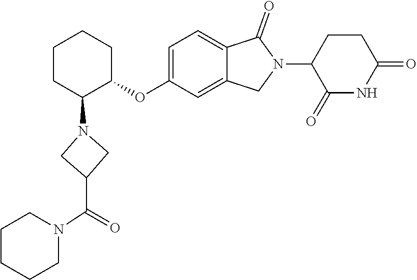 [SMILES: O=C1CCC(N2Cc3cc(O[C@H]4CCCC[C@@H]4N4CC(C(=O)N5CCCCC5)C4)ccc3C2=O)C(=O)N1] 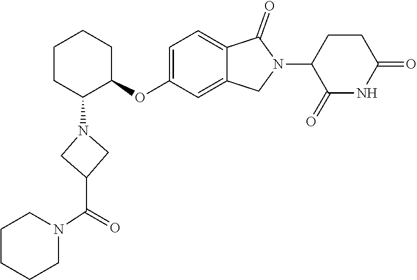 [SMILES: O=C1CCC(N2Cc3cc(O[C@@H]4CCCC[C@H]4N4CC(C(=O)N5CCCCC5)C4)ccc3C2=O)C(=O)N1] 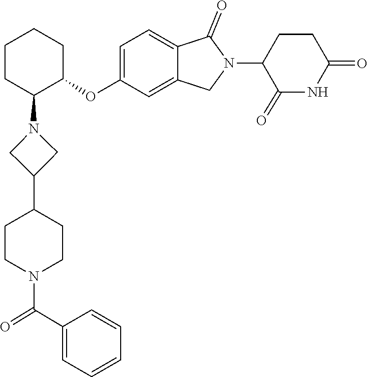 [SMILES: O=C1CCC(N2Cc3cc(O[C@H]4CCCC[C@@H]4N4CC(C5CCN(C(=O)c6ccccc6)CC5)C4)ccc3C2=O)C(=O)N1] 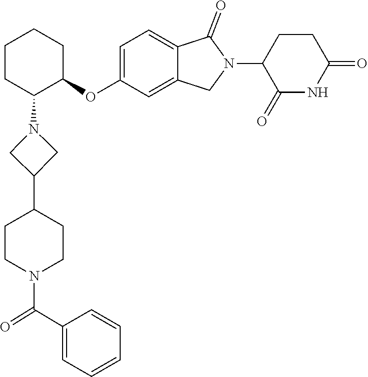 [SMILES: O=C1CCC(N2Cc3cc(O[C@@H]4CCCC[C@H]4N4CC(C5CCN(C(=O)c6ccccc6)CC5)C4)ccc3C2=O)C(=O)N1] 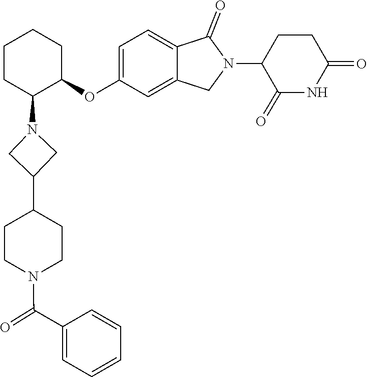 [SMILES: O=C1CCC(N2Cc3cc(O[C@@H]4CCCC[C@@H]4N4CC(C5CCN(C(=O)c6ccccc6)CC5)C4)ccc3C2=O)C(=O)N1] 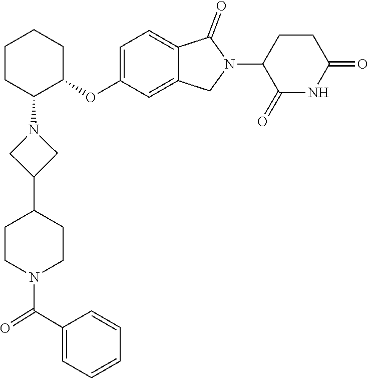 [SMILES: O=C1CCC(N2Cc3cc(O[C@H]4CCCC[C@H]4N4CC(C5CCN(C(=O)c6ccccc6)CC5)C4)ccc3C2=O)C(=O)N1] 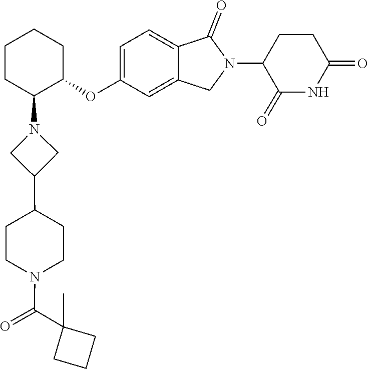 [SMILES: CC1(C(=O)N2CCC(C3CN([C@H]4CCCC[C@@H]4Oc4ccc5c(c4)CN(C4CCC(=O)NC4=O)C5=O)C3)CC2)CCC1] 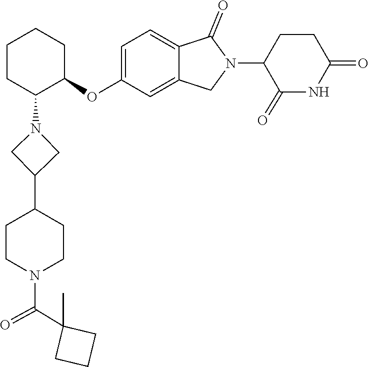 [SMILES: CC1(C(=O)N2CCC(C3CN([C@@H]4CCCC[C@H]4Oc4ccc5c(c4)CN(C4CCC(=O)NC4=O)C5=O)C3)CC2)CCC1] 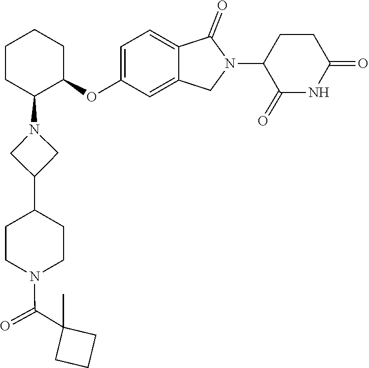 [SMILES: CC1(C(=O)N2CCC(C3CN([C@H]4CCCC[C@H]4Oc4ccc5c(c4)CN(C4CCC(=O)NC4=O)C5=O)C3)CC2)CCC1] 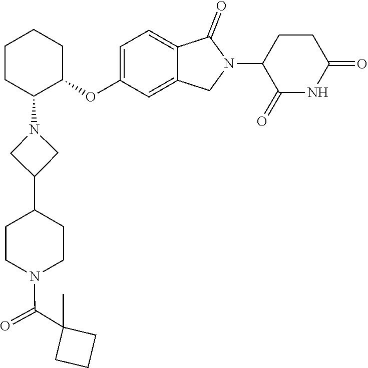 [SMILES: CC1(C(=O)N2CCC(C3CN([C@@H]4CCCC[C@@H]4Oc4ccc5c(c4)CN(C4CCC(=O)NC4=O)C5=O)C3)CC2)CCC1] 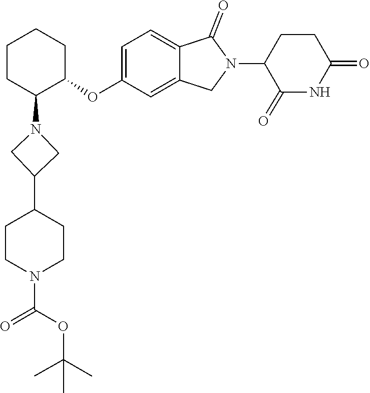 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1] 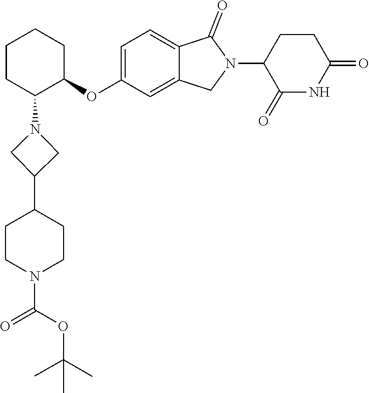 [SMILES: CC(C)(C)OC(=O)N1CCC(C2CN([C@@H]3CCCC[C@H]3Oc3ccc4c(c3)CN(C3CCC(=O)NC3=O)C4=O)C2)CC1] 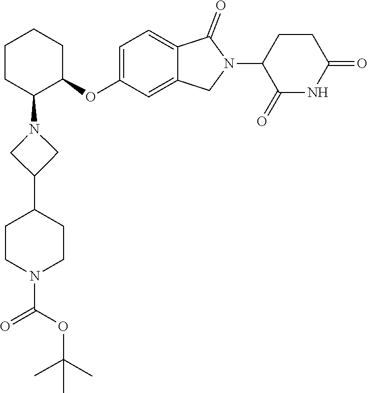 [SMILES: CC(C)(C)OC(=O)N1CCC(C2CN([C@H]3CCCC[C@H]3Oc3ccc4c(c3)CN(C3CCC(=O)NC3=O)C4=O)C2)CC1] 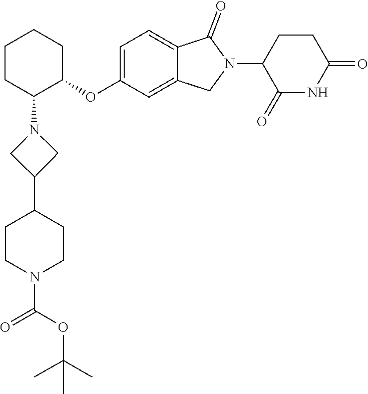 [SMILES: CC(C)(C)OC(=O)N1CCC(C2CN([C@@H]3CCCC[C@@H]3Oc3ccc4c(c3)CN(C3CCC(=O)NC3=O)C4=O)C2)CC1] 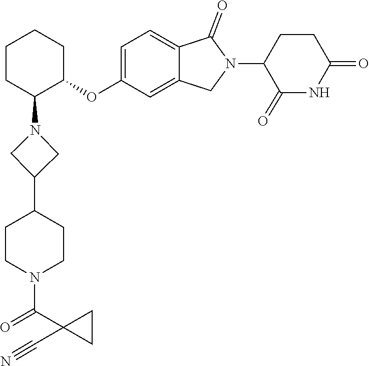 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1] 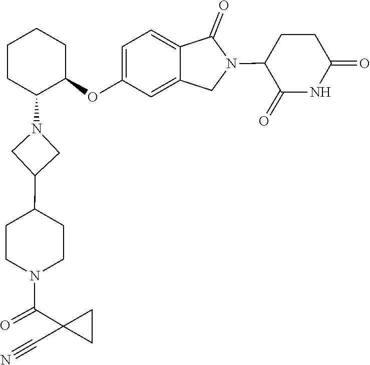 [SMILES: N#CC1(C(=O)N2CCC(C3CN([C@@H]4CCCC[C@H]4Oc4ccc5c(c4)CN(C4CCC(=O)NC4=O)C5=O)C3)CC2)CC1] 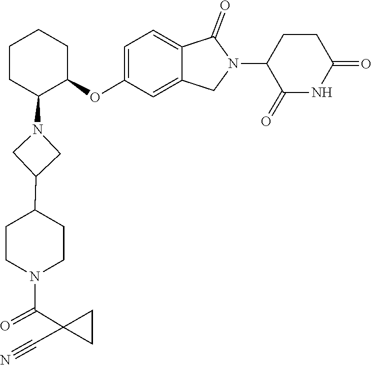 [SMILES: N#CC1(C(=O)N2CCC(C3CN([C@H]4CCCC[C@H]4Oc4ccc5c(c4)CN(C4CCC(=O)NC4=O)C5=O)C3)CC2)CC1] 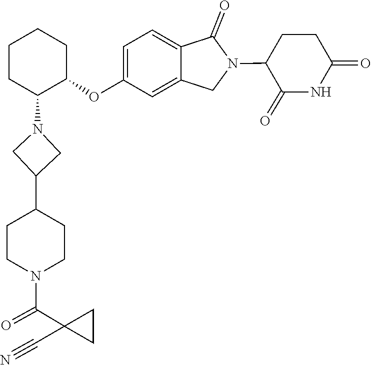 [SMILES: N#CC1(C(=O)N2CCC(C3CN([C@@H]4CCCC[C@@H]4Oc4ccc5c(c4)CN(C4CCC(=O)NC4=O)C5=O)C3)CC2)CC1] 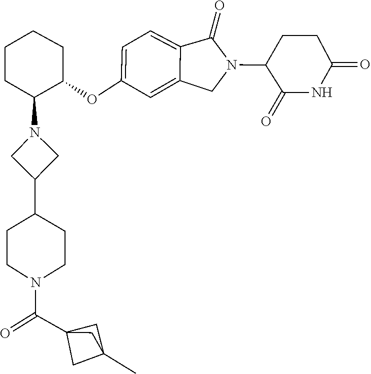 [SMILES: CC12CC(C(=O)N3CCC(C4CN([C@H]5CCCC[C@@H]5Oc5ccc6c(c5)CN(C5CCC(=O)NC5=O)C6=O)C4)CC3)(C1)C2] 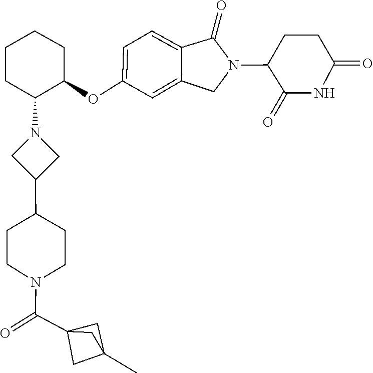 [SMILES: CC12CC(C(=O)N3CCC(C4CN([C@@H]5CCCC[C@H]5Oc5ccc6c(c5)CN(C5CCC(=O)NC5=O)C6=O)C4)CC3)(C1)C2] 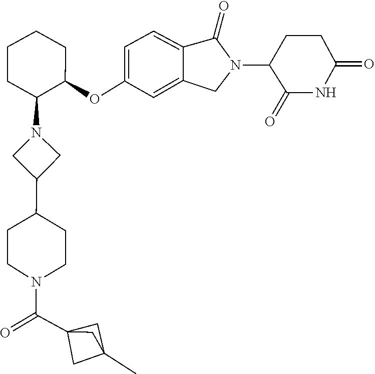 [SMILES: CC12CC(C(=O)N3CCC(C4CN([C@H]5CCCC[C@H]5Oc5ccc6c(c5)CN(C5CCC(=O)NC5=O)C6=O)C4)CC3)(C1)C2] 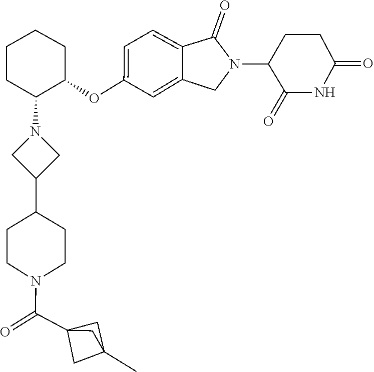 [SMILES: CC12CC(C(=O)N3CCC(C4CN([C@@H]5CCCC[C@@H]5Oc5ccc6c(c5)CN(C5CCC(=O)NC5=O)C6=O)C4)CC3)(C1)C2] 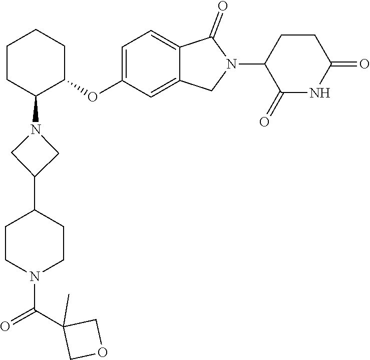 [SMILES: CC1(C(=O)N2CCC(C3CN([C@H]4CCCC[C@@H]4Oc4ccc5c(c4)CN(C4CCC(=O)NC4=O)C5=O)C3)CC2)COC1] 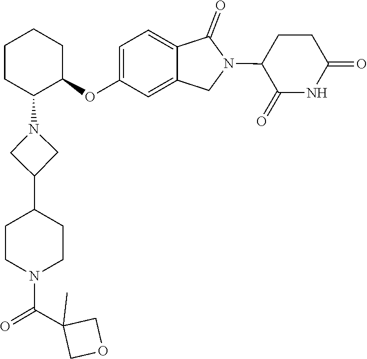 [SMILES: CC1(C(=O)N2CCC(C3CN([C@@H]4CCCC[C@H]4Oc4ccc5c(c4)CN(C4CCC(=O)NC4=O)C5=O)C3)CC2)COC1] 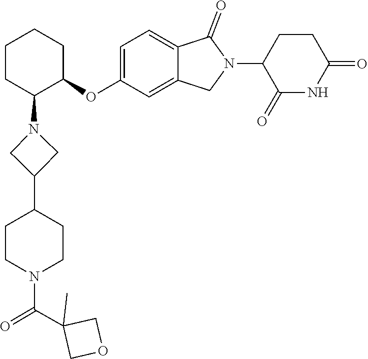 [SMILES: CC1(C(=O)N2CCC(C3CN([C@H]4CCCC[C@H]4Oc4ccc5c(c4)CN(C4CCC(=O)NC4=O)C5=O)C3)CC2)COC1] 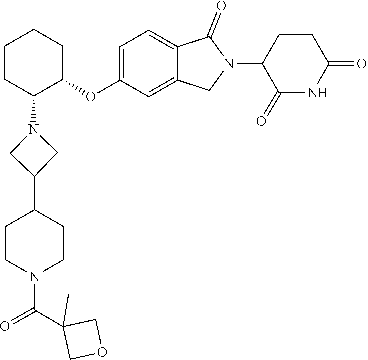 [SMILES: CC1(C(=O)N2CCC(C3CN([C@@H]4CCCC[C@@H]4Oc4ccc5c(c4)CN(C4CCC(=O)NC4=O)C5=O)C3)CC2)COC1] 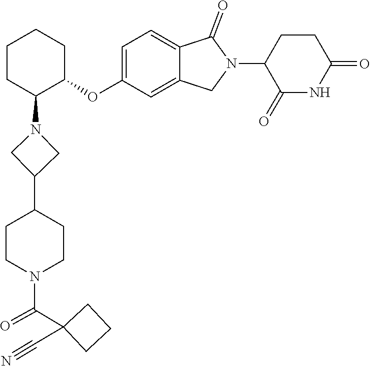 [SMILES: N#CC1(C(=O)N2CCC(C3CN([C@H]4CCCC[C@@H]4Oc4ccc5c(c4)CN(C4CCC(=O)NC4=O)C5=O)C3)CC2)CCC1] 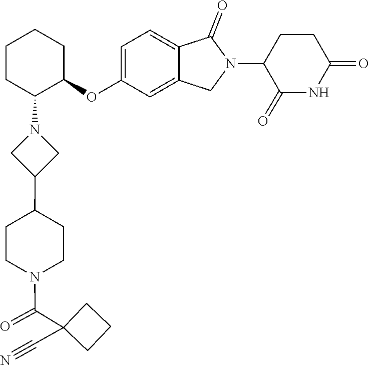 [SMILES: N#CC1(C(=O)N2CCC(C3CN([C@@H]4CCCC[C@H]4Oc4ccc5c(c4)CN(C4CCC(=O)NC4=O)C5=O)C3)CC2)CCC1] 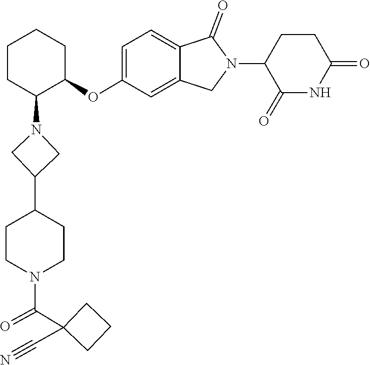 [SMILES: N#CC1(C(=O)N2CCC(C3CN([C@H]4CCCC[C@H]4Oc4ccc5c(c4)CN(C4CCC(=O)NC4=O)C5=O)C3)CC2)CCC1] 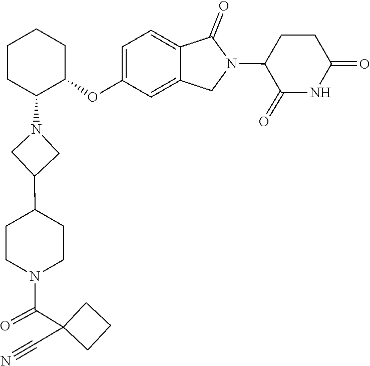 [SMILES: N#CC1(C(=O)N2CCC(C3CN([C@@H]4CCCC[C@@H]4Oc4ccc5c(c4)CN(C4CCC(=O)NC4=O)C5=O)C3)CC2)CCC1] 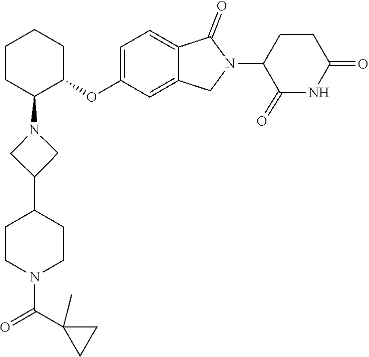 [SMILES: CC1(C(=O)N2CCC(C3CN([C@H]4CCCC[C@@H]4Oc4ccc5c(c4)CN(C4CCC(=O)NC4=O)C5=O)C3)CC2)CC1] 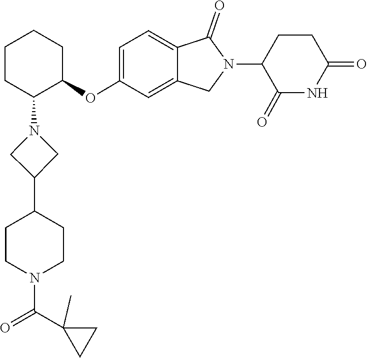 [SMILES: CC1(C(=O)N2CCC(C3CN([C@@H]4CCCC[C@H]4Oc4ccc5c(c4)CN(C4CCC(=O)NC4=O)C5=O)C3)CC2)CC1] 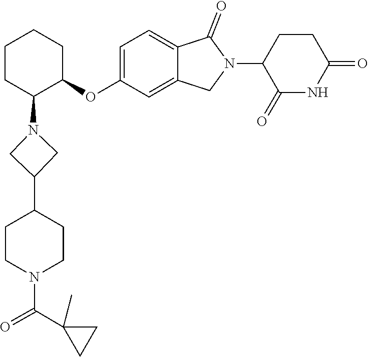 [SMILES: CC1(C(=O)N2CCC(C3CN([C@H]4CCCC[C@H]4Oc4ccc5c(c4)CN(C4CCC(=O)NC4=O)C5=O)C3)CC2)CC1] 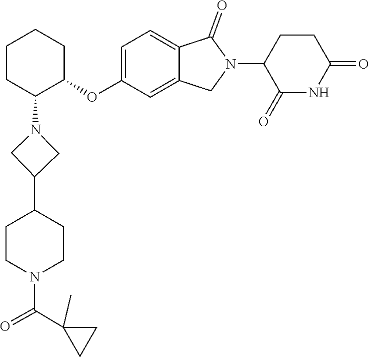 [SMILES: CC1(C(=O)N2CCC(C3CN([C@@H]4CCCC[C@@H]4Oc4ccc5c(c4)CN(C4CCC(=O)NC4=O)C5=O)C3)CC2)CC1] 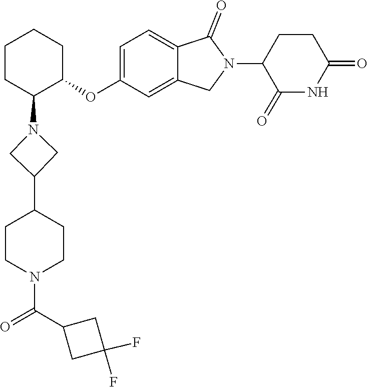 [SMILES: O=C1CCC(N2Cc3cc(O[C@H]4CCCC[C@@H]4N4CC(C5CCN(C(=O)C6CC(F)(F)C6)CC5)C4)ccc3C2=O)C(=O)N1] 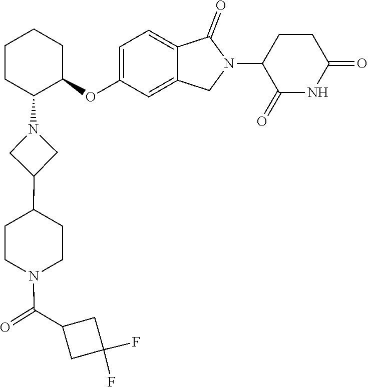 [SMILES: O=C1CCC(N2Cc3cc(O[C@@H]4CCCC[C@H]4N4CC(C5CCN(C(=O)C6CC(F)(F)C6)CC5)C4)ccc3C2=O)C(=O)N1] 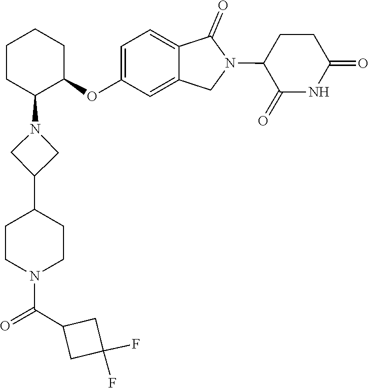 [SMILES: O=C1CCC(N2Cc3cc(O[C@@H]4CCCC[C@@H]4N4CC(C5CCN(C(=O)C6CC(F)(F)C6)CC5)C4)ccc3C2=O)C(=O)N1] 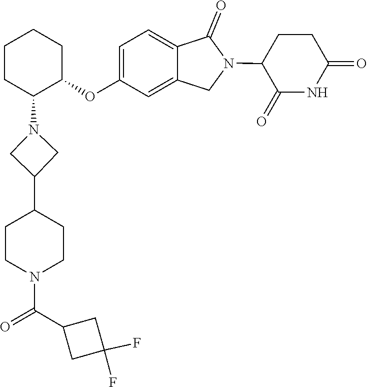 [SMILES: O=C1CCC(N2Cc3cc(O[C@H]4CCCC[C@H]4N4CC(C5CCN(C(=O)C6CC(F)(F)C6)CC5)C4)ccc3C2=O)C(=O)N1] 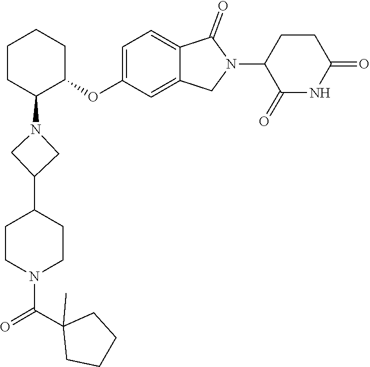 [SMILES: CC1(C(=O)N2CCC(C3CN([C@H]4CCCC[C@@H]4Oc4ccc5c(c4)CN(C4CCC(=O)NC4=O)C5=O)C3)CC2)CCCC1] 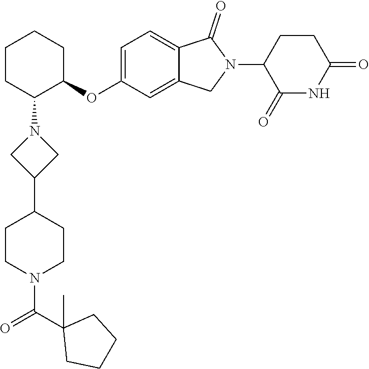 [SMILES: CC1(C(=O)N2CCC(C3CN([C@@H]4CCCC[C@H]4Oc4ccc5c(c4)CN(C4CCC(=O)NC4=O)C5=O)C3)CC2)CCCC1] 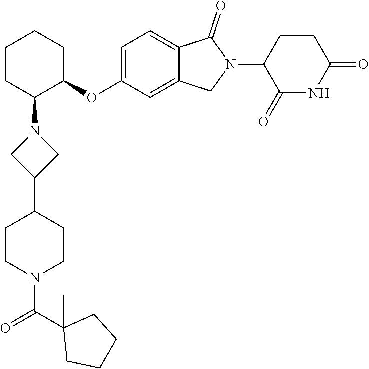 [SMILES: CC1(C(=O)N2CCC(C3CN([C@H]4CCCC[C@H]4Oc4ccc5c(c4)CN(C4CCC(=O)NC4=O)C5=O)C3)CC2)CCCC1] 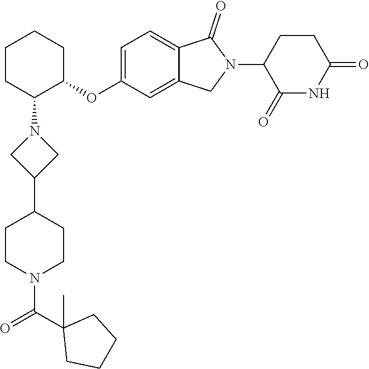 [SMILES: CC1(C(=O)N2CCC(C3CN([C@@H]4CCCC[C@@H]4Oc4ccc5c(c4)CN(C4CCC(=O)NC4=O)C5=O)C3)CC2)CCCC1] 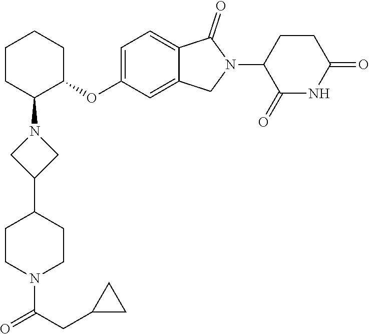 [SMILES: O=C1CCC(N2Cc3cc(O[C@H]4CCCC[C@@H]4N4CC(C5CCN(C(=O)CC6CC6)CC5)C4)ccc3C2=O)C(=O)N1] 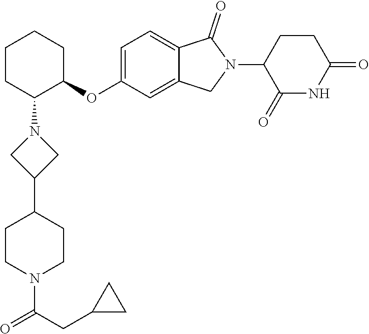 [SMILES: O=C1CCC(N2Cc3cc(O[C@@H]4CCCC[C@H]4N4CC(C5CCN(C(=O)CC6CC6)CC5)C4)ccc3C2=O)C(=O)N1] 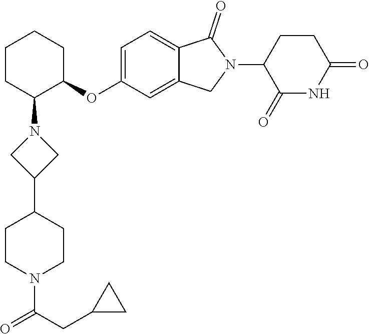 [SMILES: O=C1CCC(N2Cc3cc(O[C@@H]4CCCC[C@@H]4N4CC(C5CCN(C(=O)CC6CC6)CC5)C4)ccc3C2=O)C(=O)N1] 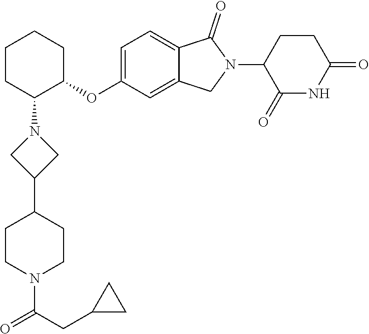 [SMILES: O=C1CCC(N2Cc3cc(O[C@H]4CCCC[C@H]4N4CC(C5CCN(C(=O)CC6CC6)CC5)C4)ccc3C2=O)C(=O)N1] 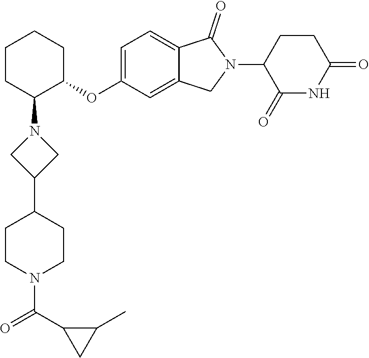 [SMILES: CC1CC1C(=O)N1CCC(C2CN([C@H]3CCCC[C@@H]3Oc3ccc4c(c3)CN(C3CCC(=O)NC3=O)C4=O)C2)CC1] 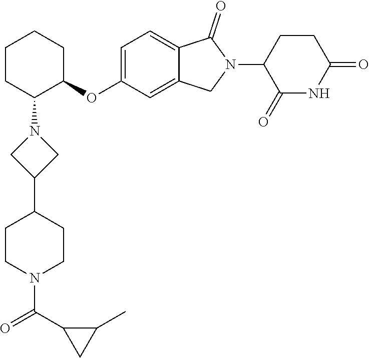 [SMILES: CC1CC1C(=O)N1CCC(C2CN([C@@H]3CCCC[C@H]3Oc3ccc4c(c3)CN(C3CCC(=O)NC3=O)C4=O)C2)CC1] 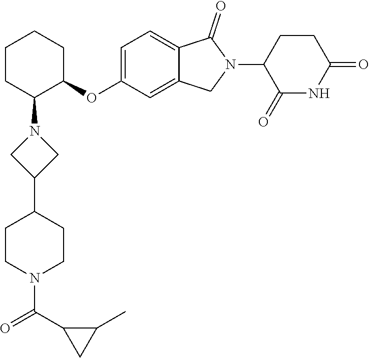 [SMILES: CC1CC1C(=O)N1CCC(C2CN([C@H]3CCCC[C@H]3Oc3ccc4c(c3)CN(C3CCC(=O)NC3=O)C4=O)C2)CC1] 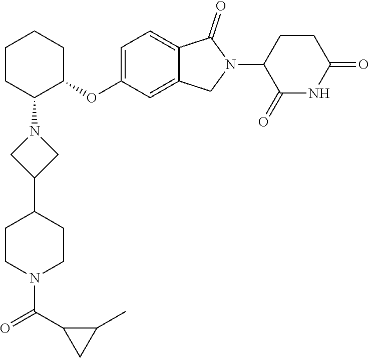 [SMILES: CC1CC1C(=O)N1CCC(C2CN([C@@H]3CCCC[C@@H]3Oc3ccc4c(c3)CN(C3CCC(=O)NC3=O)C4=O)C2)CC1] 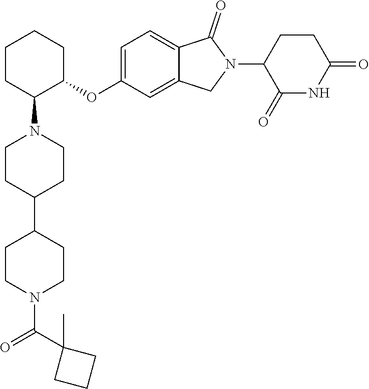 [SMILES: CC1(C(=O)N2CCC(C3CCN([C@H]4CCCC[C@@H]4Oc4ccc5c(c4)CN(C4CCC(=O)NC4=O)C5=O)CC3)CC2)CCC1] 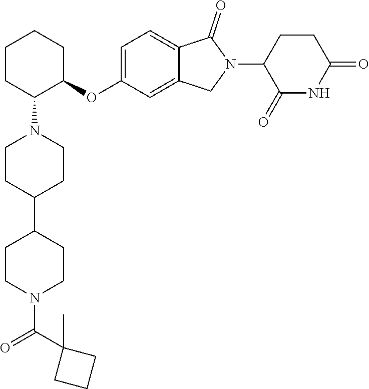 [SMILES: CC1(C(=O)N2CCC(C3CCN([C@@H]4CCCC[C@H]4Oc4ccc5c(c4)CN(C4CCC(=O)NC4=O)C5=O)CC3)CC2)CCC1] 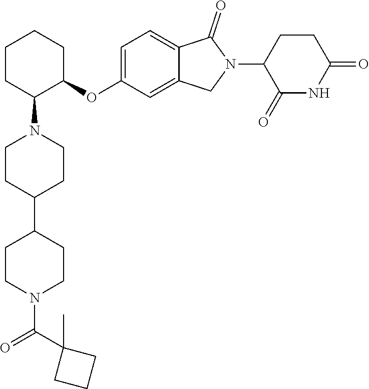 [SMILES: CC1(C(=O)N2CCC(C3CCN([C@H]4CCCC[C@H]4Oc4ccc5c(c4)CN(C4CCC(=O)NC4=O)C5=O)CC3)CC2)CCC1] 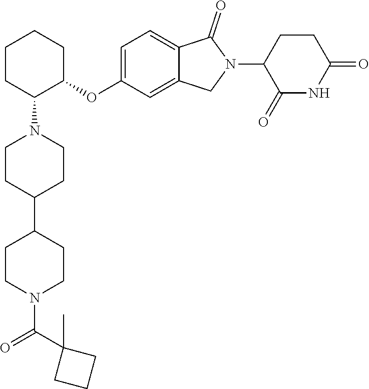 [SMILES: CC1(C(=O)N2CCC(C3CCN([C@@H]4CCCC[C@@H]4Oc4ccc5c(c4)CN(C4CCC(=O)NC4=O)C5=O)CC3)CC2)CCC1] 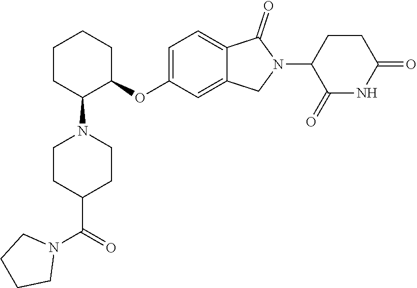 [SMILES: O=C1CCC(N2Cc3cc(O[C@@H]4CCCC[C@@H]4N4CCC(C(=O)N5CCCC5)CC4)ccc3C2=O)C(=O)N1] 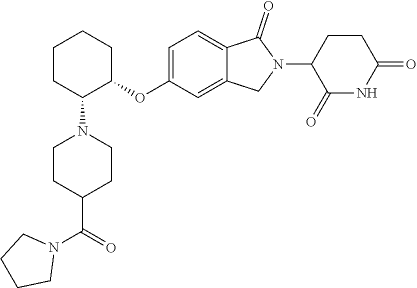 [SMILES: O=C1CCC(N2Cc3cc(O[C@H]4CCCC[C@H]4N4CCC(C(=O)N5CCCC5)CC4)ccc3C2=O)C(=O)N1] 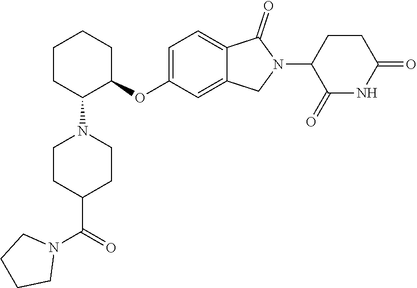 [SMILES: O=C1CCC(N2Cc3cc(O[C@@H]4CCCC[C@H]4N4CCC(C(=O)N5CCCC5)CC4)ccc3C2=O)C(=O)N1] 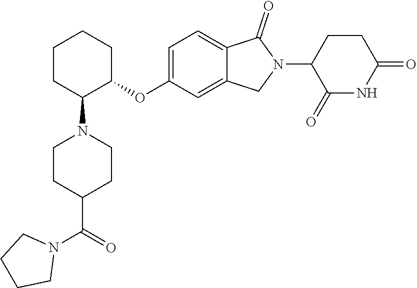 [SMILES: O=C1CCC(N2Cc3cc(O[C@H]4CCCC[C@@H]4N4CCC(C(=O)N5CCCC5)CC4)ccc3C2=O)C(=O)N1] 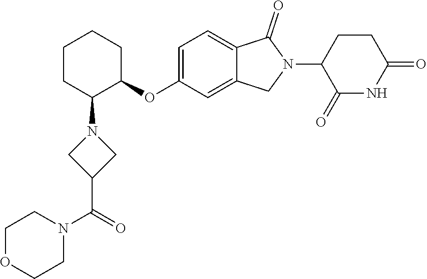 [SMILES: O=C1CCC(N2Cc3cc(O[C@@H]4CCCC[C@@H]4N4CC(C(=O)N5CCOCC5)C4)ccc3C2=O)C(=O)N1] 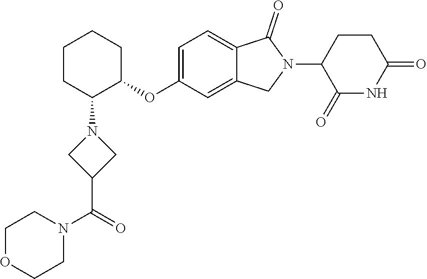 [SMILES: O=C1CCC(N2Cc3cc(O[C@H]4CCCC[C@H]4N4CC(C(=O)N5CCOCC5)C4)ccc3C2=O)C(=O)N1] 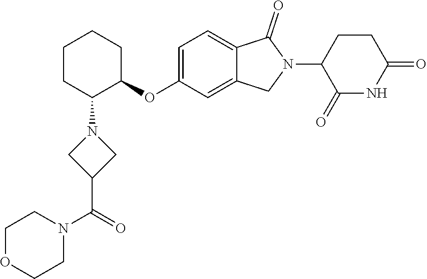 [SMILES: O=C1CCC(N2Cc3cc(O[C@@H]4CCCC[C@H]4N4CC(C(=O)N5CCOCC5)C4)ccc3C2=O)C(=O)N1] 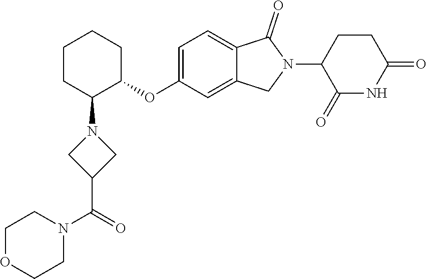 [SMILES: O=C1CCC(N2Cc3cc(O[C@H]4CCCC[C@@H]4N4CC(C(=O)N5CCOCC5)C4)ccc3C2=O)C(=O)N1] 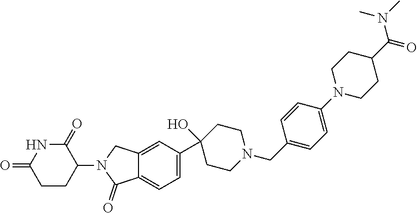 [SMILES: CN(C)C(=O)C1CCN(c2ccc(CN3CCC(O)(c4ccc5c(c4)CN(C4CCC(=O)NC4=O)C5=O)CC3)cc2)CC1] 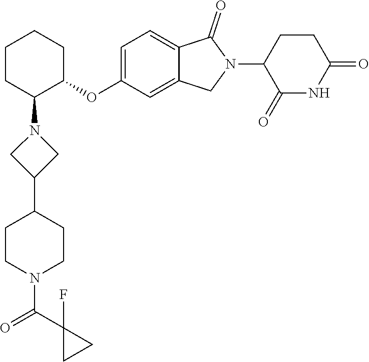 [SMILES: O=C1CCC(N2Cc3cc(O[C@H]4CCCC[C@@H]4N4CC(C5CCN(C(=O)C6(F)CC6)CC5)C4)ccc3C2=O)C(=O)N1] 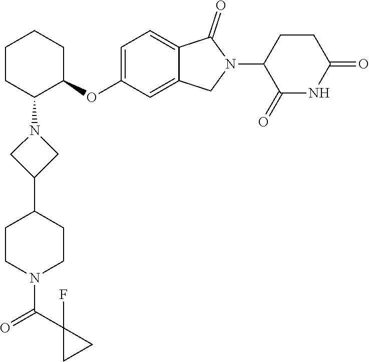 [SMILES: O=C1CCC(N2Cc3cc(O[C@@H]4CCCC[C@H]4N4CC(C5CCN(C(=O)C6(F)CC6)CC5)C4)ccc3C2=O)C(=O)N1] 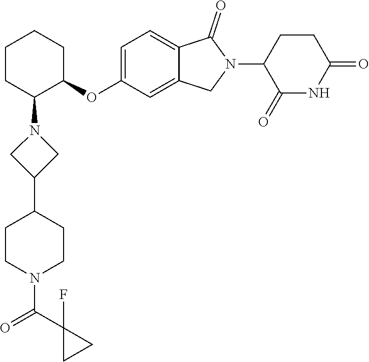 [SMILES: O=C1CCC(N2Cc3cc(O[C@@H]4CCCC[C@@H]4N4CC(C5CCN(C(=O)C6(F)CC6)CC5)C4)ccc3C2=O)C(=O)N1] 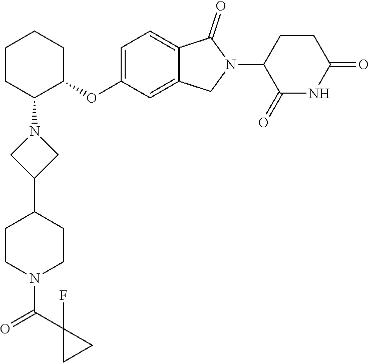 [SMILES: O=C1CCC(N2Cc3cc(O[C@H]4CCCC[C@H]4N4CC(C5CCN(C(=O)C6(F)CC6)CC5)C4)ccc3C2=O)C(=O)N1] 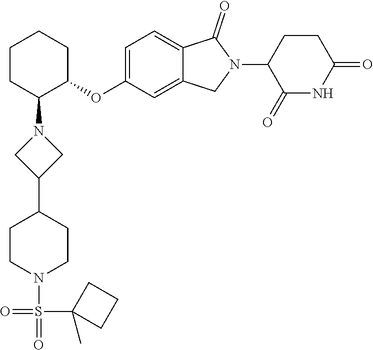 [SMILES: CC1(S(=O)(=O)N2CCC(C3CN([C@H]4CCCC[C@@H]4Oc4ccc5c(c4)CN(C4CCC(=O)NC4=O)C5=O)C3)CC2)CCC1] 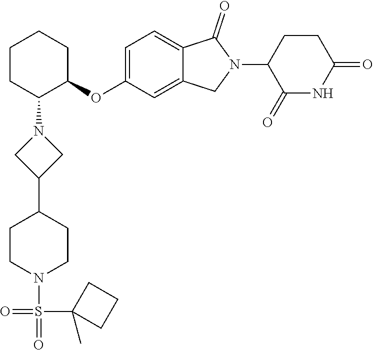 [SMILES: CC1(S(=O)(=O)N2CCC(C3CN([C@@H]4CCCC[C@H]4Oc4ccc5c(c4)CN(C4CCC(=O)NC4=O)C5=O)C3)CC2)CCC1] 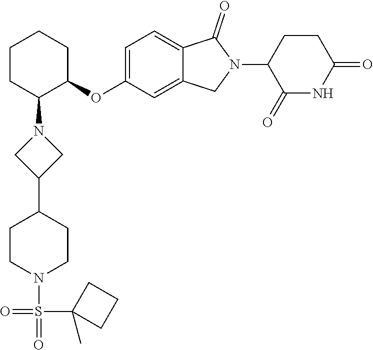 [SMILES: CC1(S(=O)(=O)N2CCC(C3CN([C@H]4CCCC[C@H]4Oc4ccc5c(c4)CN(C4CCC(=O)NC4=O)C5=O)C3)CC2)CCC1] 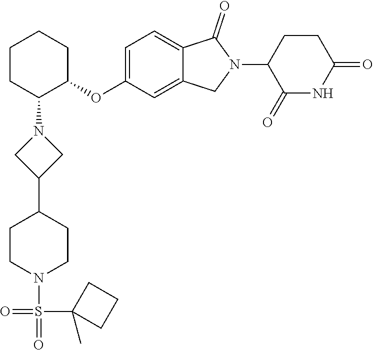 [SMILES: CC1(S(=O)(=O)N2CCC(C3CN([C@@H]4CCCC[C@@H]4Oc4ccc5c(c4)CN(C4CCC(=O)NC4=O)C5=O)C3)CC2)CCC1] 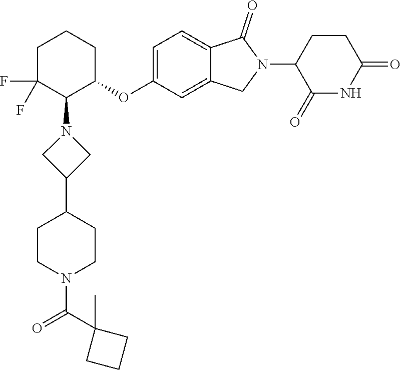 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCC1] 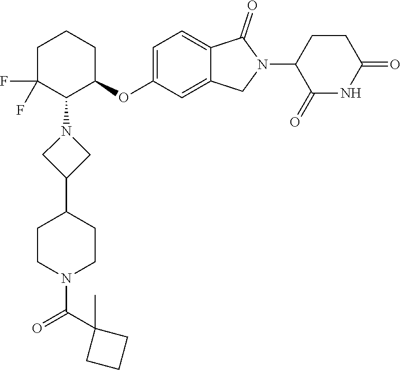 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)CCC1] 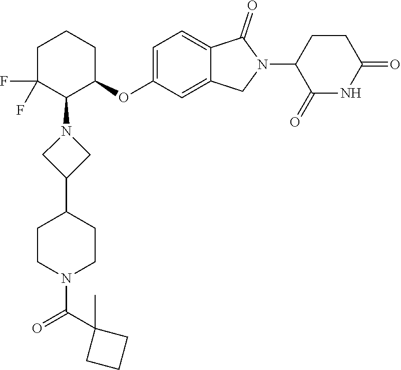 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)CCC1] 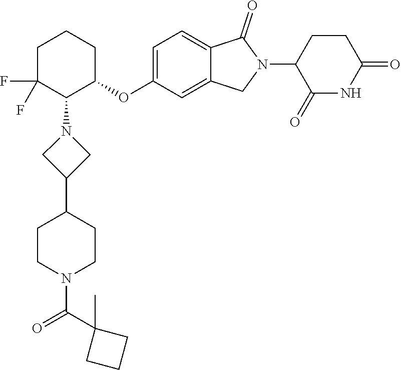 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)CCC1] 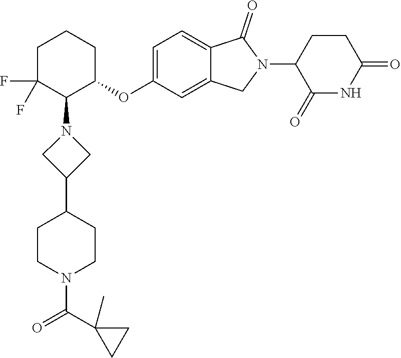 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CC1] 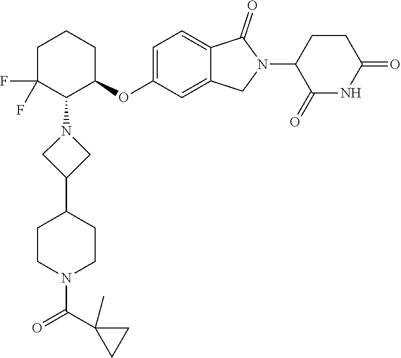 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)CC1] 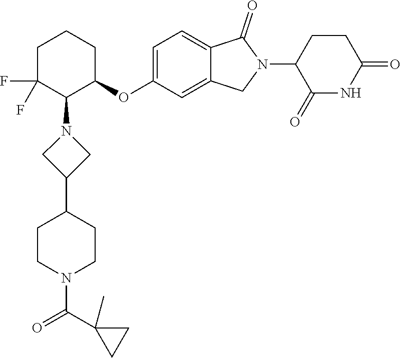 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)CC1] 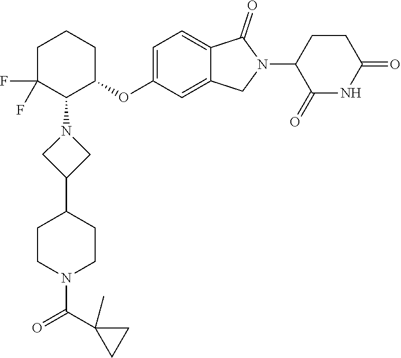 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)CC1] 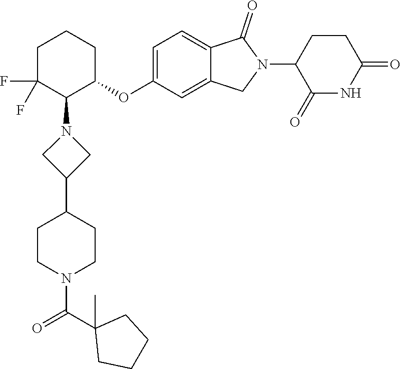 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)CCCC1] 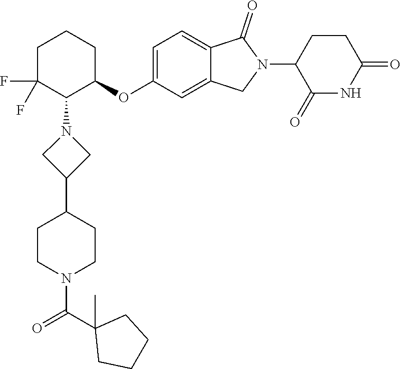 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)CCCC1] 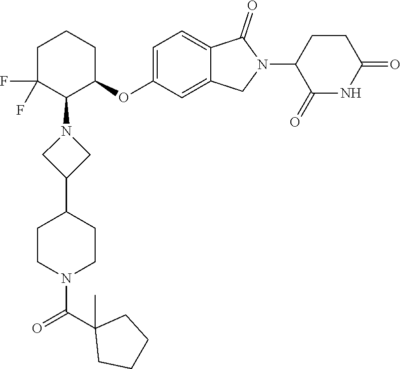 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)CCCC1] 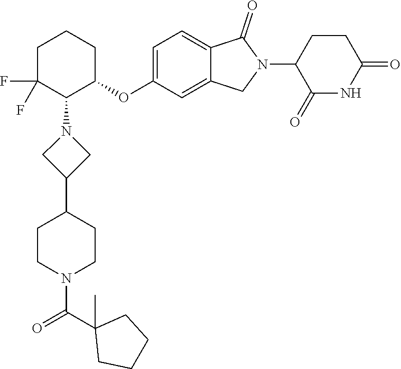 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)CCCC1] 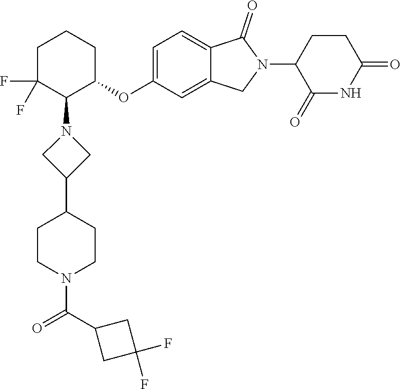 [SMILES: O=C1CCC(N2Cc3cc(O[C@H]4CCCC(F)(F)[C@@H]4N4CC(C5CCN(C(=O)C6CC(F)(F)C6)CC5)C4)ccc3C2=O)C(=O)N1] 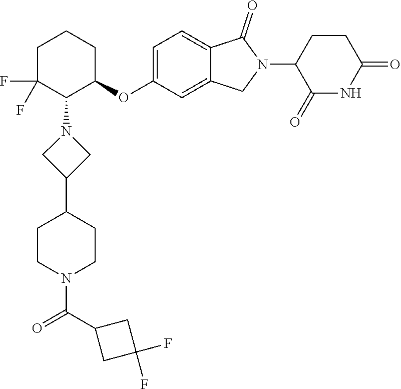 [SMILES: O=C1CCC(N2Cc3cc(O[C@@H]4CCCC(F)(F)[C@H]4N4CC(C5CCN(C(=O)C6CC(F)(F)C6)CC5)C4)ccc3C2=O)C(=O)N1] 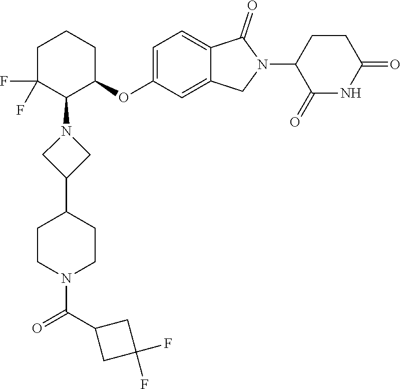 [SMILES: O=C1CCC(N2Cc3cc(O[C@@H]4CCCC(F)(F)[C@@H]4N4CC(C5CCN(C(=O)C6CC(F)(F)C6)CC5)C4)ccc3C2=O)C(=O)N1] 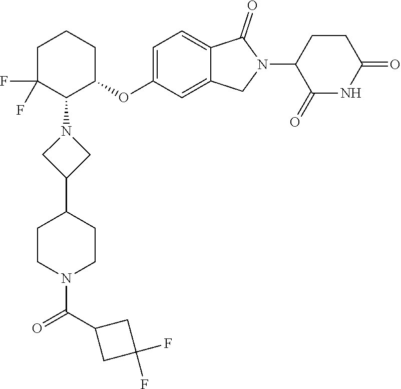 [SMILES: O=C1CCC(N2Cc3cc(O[C@H]4CCCC(F)(F)[C@H]4N4CC(C5CCN(C(=O)C6CC(F)(F)C6)CC5)C4)ccc3C2=O)C(=O)N1] 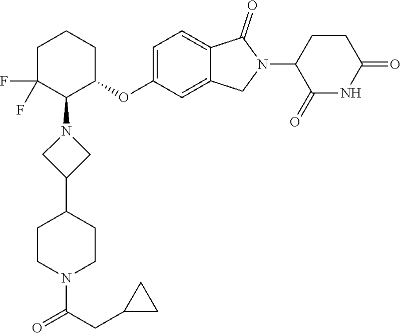 [SMILES: O=C1CCC(N2Cc3cc(O[C@H]4CCCC(F)(F)[C@@H]4N4CC(C5CCN(C(=O)CC6CC6)CC5)C4)ccc3C2=O)C(=O)N1] 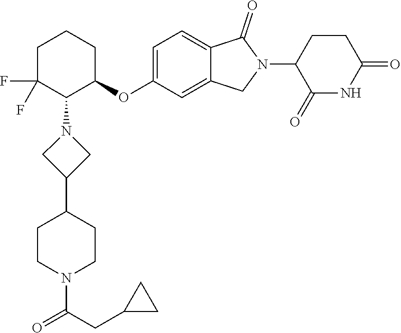 [SMILES: O=C1CCC(N2Cc3cc(O[C@@H]4CCCC(F)(F)[C@H]4N4CC(C5CCN(C(=O)CC6CC6)CC5)C4)ccc3C2=O)C(=O)N1] 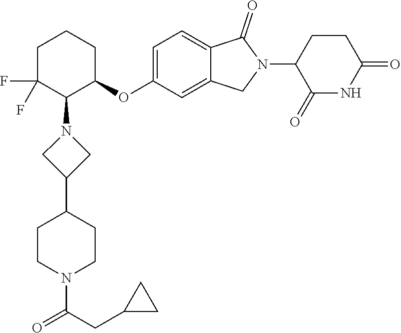 [SMILES: O=C1CCC(N2Cc3cc(O[C@@H]4CCCC(F)(F)[C@@H]4N4CC(C5CCN(C(=O)CC6CC6)CC5)C4)ccc3C2=O)C(=O)N1] 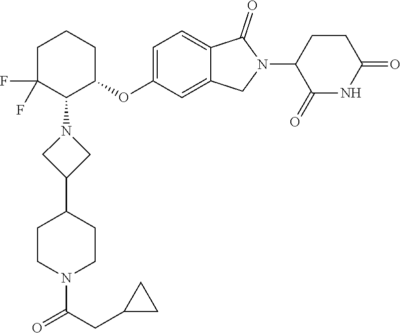 [SMILES: O=C1CCC(N2Cc3cc(O[C@H]4CCCC(F)(F)[C@H]4N4CC(C5CCN(C(=O)CC6CC6)CC5)C4)ccc3C2=O)C(=O)N1] 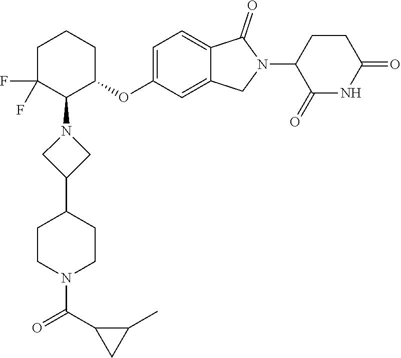 [SMILES: CC1CC1C(=O)N1CCC(C2CN([C@@H]3[C@@H](Oc4ccc5c(c4)CN(C4CCC(=O)NC4=O)C5=O)CCCC3(F)F)C2)CC1] 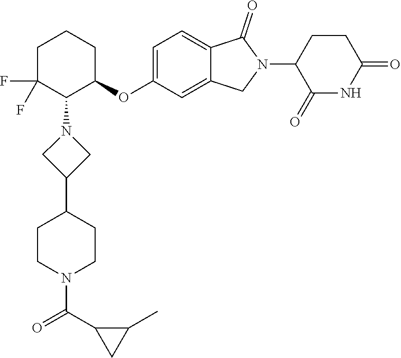 [SMILES: CC1CC1C(=O)N1CCC(C2CN([C@H]3[C@H](Oc4ccc5c(c4)CN(C4CCC(=O)NC4=O)C5=O)CCCC3(F)F)C2)CC1] 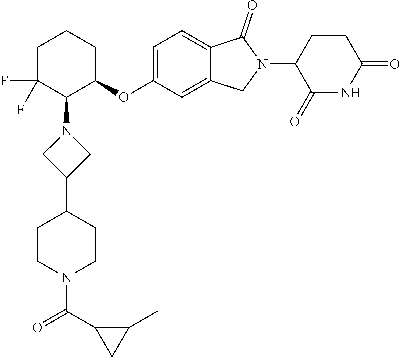 [SMILES: CC1CC1C(=O)N1CCC(C2CN([C@@H]3[C@H](Oc4ccc5c(c4)CN(C4CCC(=O)NC4=O)C5=O)CCCC3(F)F)C2)CC1] 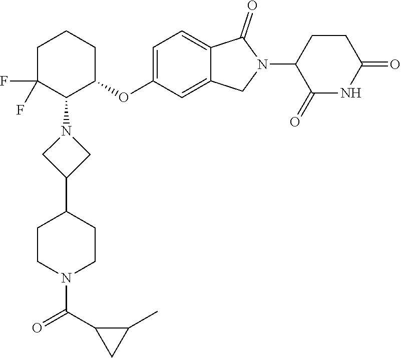 [SMILES: CC1CC1C(=O)N1CCC(C2CN([C@H]3[C@@H](Oc4ccc5c(c4)CN(C4CCC(=O)NC4=O)C5=O)CCCC3(F)F)C2)CC1] 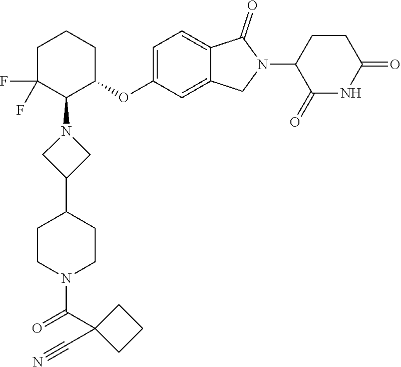 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CCC1] 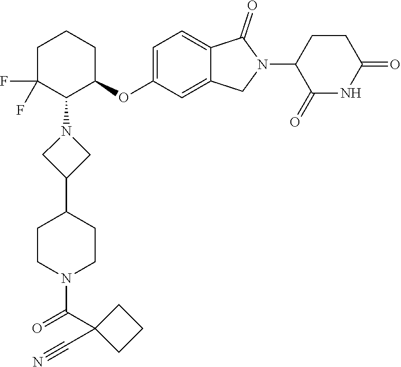 [SMILES: N#CC1(C(=O)N2CCC(C3CN([C@H]4[C@H](Oc5ccc6c(c5)CN(C5CCC(=O)NC5=O)C6=O)CCCC4(F)F)C3)CC2)CCC1] 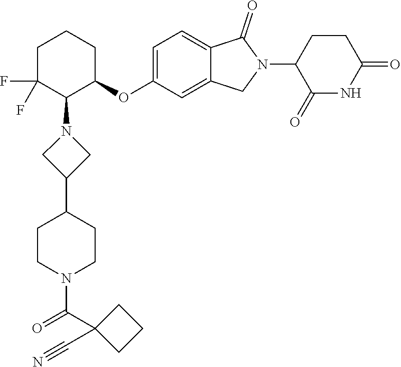 [SMILES: N#CC1(C(=O)N2CCC(C3CN([C@@H]4[C@H](Oc5ccc6c(c5)CN(C5CCC(=O)NC5=O)C6=O)CCCC4(F)F)C3)CC2)CCC1] 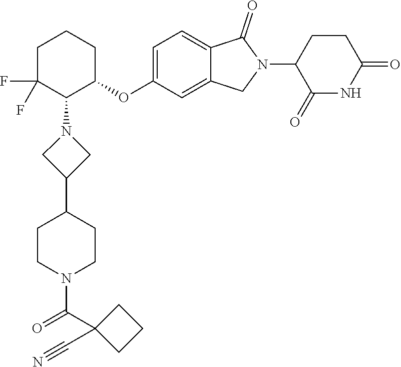 [SMILES: N#CC1(C(=O)N2CCC(C3CN([C@H]4[C@@H](Oc5ccc6c(c5)CN(C5CCC(=O)NC5=O)C6=O)CCCC4(F)F)C3)CC2)CCC1] 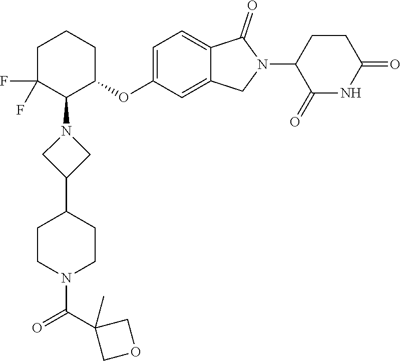 [SMILES: CC1(C(=O)N2CCC(C3CN([C@@H]4[C@@H](Oc5ccc6c(c5)CN(C5CCC(=O)NC5=O)C6=O)CCCC4(F)F)C3)CC2)COC1] 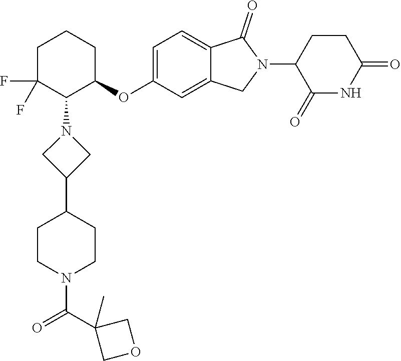 [SMILES: CC1(C(=O)N2CCC(C3CN([C@H]4[C@H](Oc5ccc6c(c5)CN(C5CCC(=O)NC5=O)C6=O)CCCC4(F)F)C3)CC2)COC1] 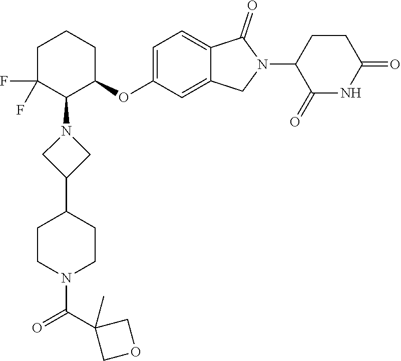 [SMILES: CC1(C(=O)N2CCC(C3CN([C@@H]4[C@H](Oc5ccc6c(c5)CN(C5CCC(=O)NC5=O)C6=O)CCCC4(F)F)C3)CC2)COC1] 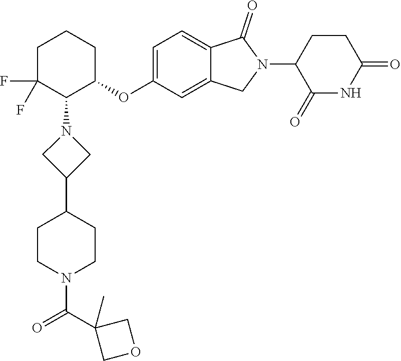 [SMILES: CC1(C(=O)N2CCC(C3CN([C@H]4[C@@H](Oc5ccc6c(c5)CN(C5CCC(=O)NC5=O)C6=O)CCCC4(F)F)C3)CC2)COC1] 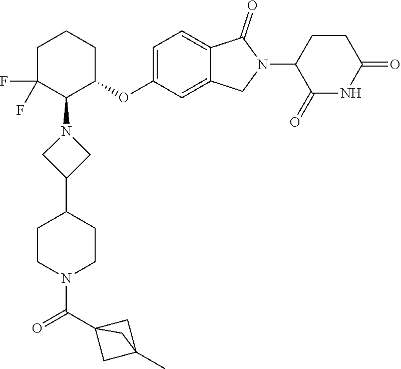 [SMILES: CC12CC(C(=O)N3CCC(C4CN([C@@H]5[C@@H](Oc6ccc7c(c6)CN(C6CCC(=O)NC6=O)C7=O)CCCC5(F)F)C4)CC3)(C1)C2] 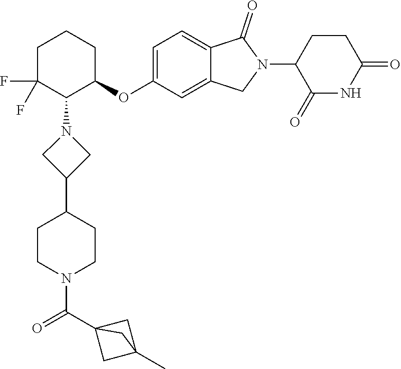 [SMILES: CC12CC(C(=O)N3CCC(C4CN([C@H]5[C@H](Oc6ccc7c(c6)CN(C6CCC(=O)NC6=O)C7=O)CCCC5(F)F)C4)CC3)(C1)C2] 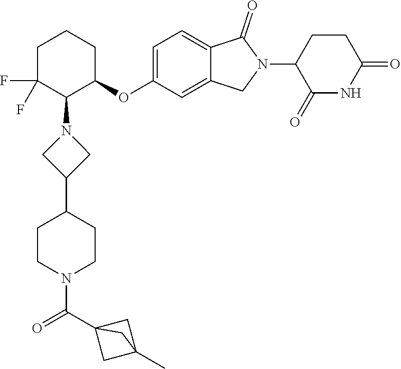 [SMILES: CC12CC(C(=O)N3CCC(C4CN([C@@H]5[C@H](Oc6ccc7c(c6)CN(C6CCC(=O)NC6=O)C7=O)CCCC5(F)F)C4)CC3)(C1)C2] 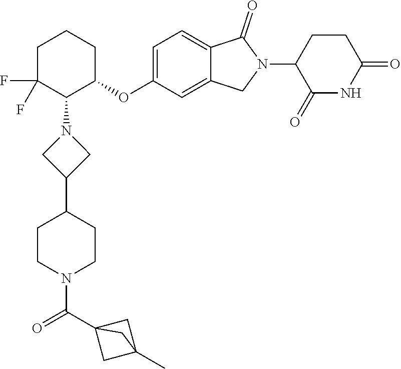 [SMILES: CC12CC(C(=O)N3CCC(C4CN([C@H]5[C@@H](Oc6ccc7c(c6)CN(C6CCC(=O)NC6=O)C7=O)CCCC5(F)F)C4)CC3)(C1)C2] 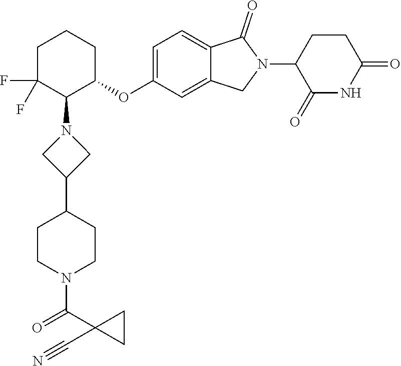 [SMILES: N#CC1(C(=O)N2CCC(C3CN([C@@H]4[C@@H](Oc5ccc6c(c5)CN(C5CCC(=O)NC5=O)C6=O)CCCC4(F)F)C3)CC2)CC1] 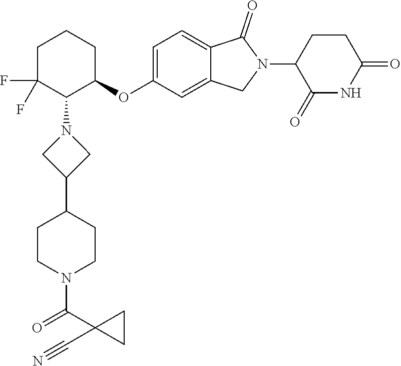 [SMILES: N#CC1(C(=O)N2CCC(C3CN([C@H]4[C@H](Oc5ccc6c(c5)CN(C5CCC(=O)NC5=O)C6=O)CCCC4(F)F)C3)CC2)CC1] 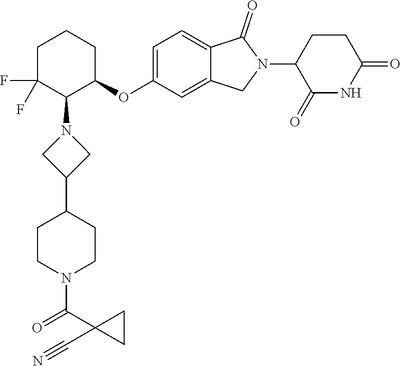 [SMILES: N#CC1(C(=O)N2CCC(C3CN([C@@H]4[C@H](Oc5ccc6c(c5)CN(C5CCC(=O)NC5=O)C6=O)CCCC4(F)F)C3)CC2)CC1] 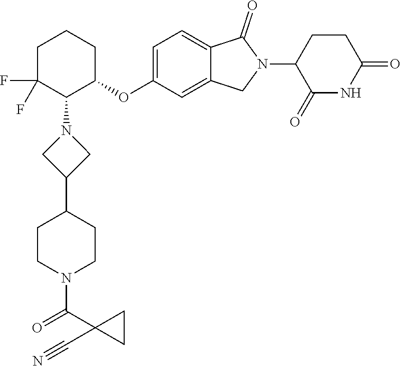 [SMILES: N#CC1(C(=O)N2CCC(C3CN([C@H]4[C@@H](Oc5ccc6c(c5)CN(C5CCC(=O)NC5=O)C6=O)CCCC4(F)F)C3)CC2)CC1] 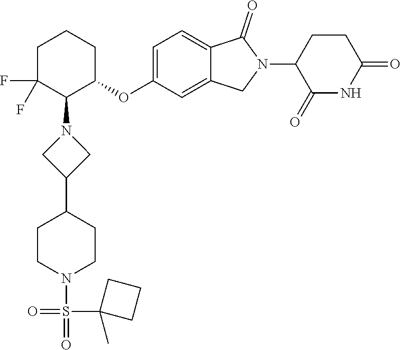 [SMILES: CC1(S(=O)(=O)N2CCC(C3CN([C@@H]4[C@@H](Oc5ccc6c(c5)CN(C5CCC(=O)NC5=O)C6=O)CCCC4(F)F)C3)CC2)CCC1] 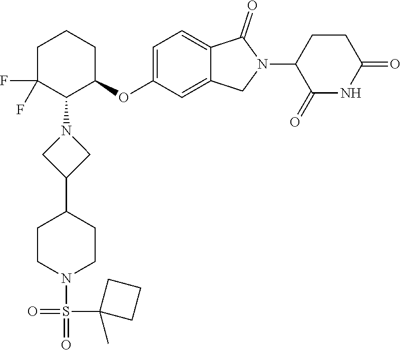 [SMILES: CC1(S(=O)(=O)N2CCC(C3CN([C@H]4[C@H](Oc5ccc6c(c5)CN(C5CCC(=O)NC5=O)C6=O)CCCC4(F)F)C3)CC2)CCC1] 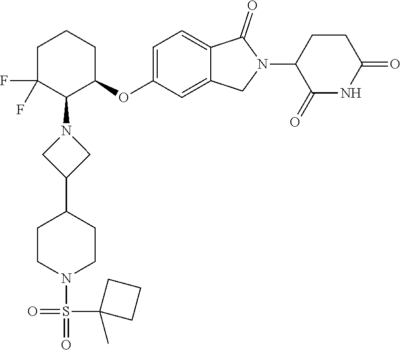 [SMILES: CC1(S(=O)(=O)N2CCC(C3CN([C@@H]4[C@H](Oc5ccc6c(c5)CN(C5CCC(=O)NC5=O)C6=O)CCCC4(F)F)C3)CC2)CCC1] 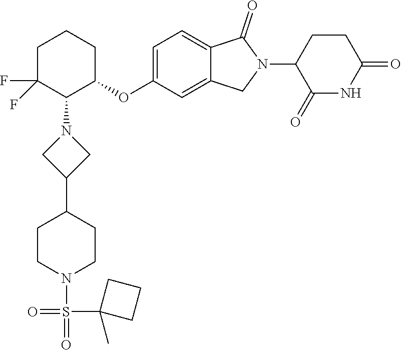 [SMILES: CC1(S(=O)(=O)N2CCC(C3CN([C@H]4[C@@H](Oc5ccc6c(c5)CN(C5CCC(=O)NC5=O)C6=O)CCCC4(F)F)C3)CC2)CCC1] 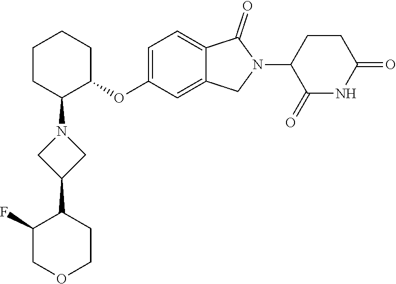 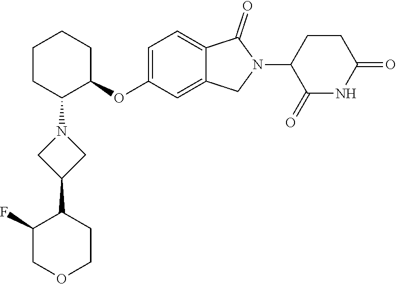 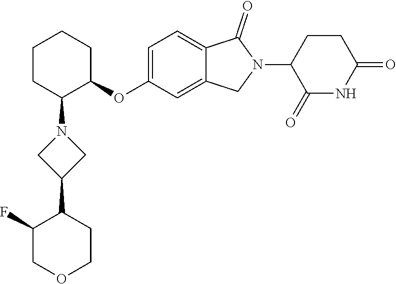 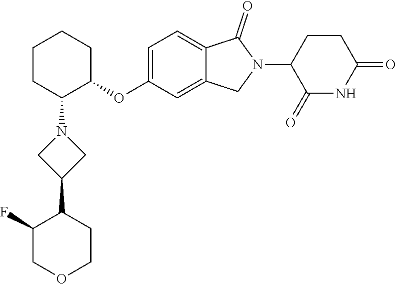 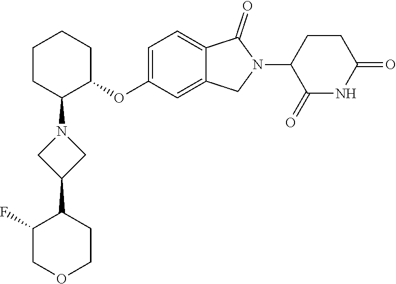 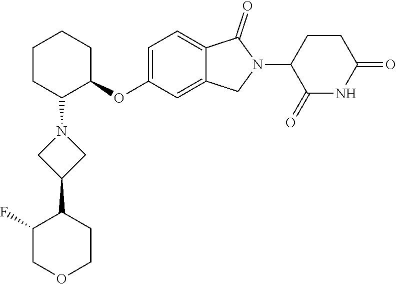 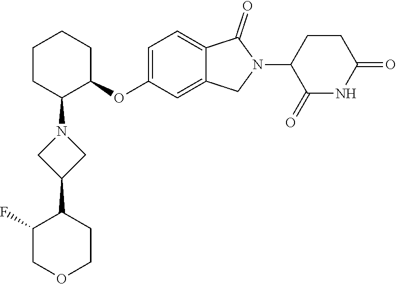 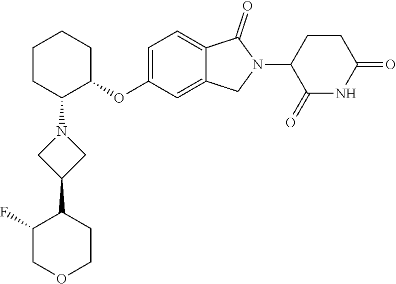 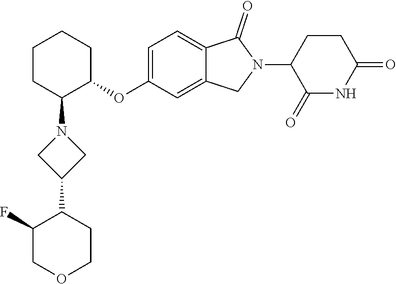 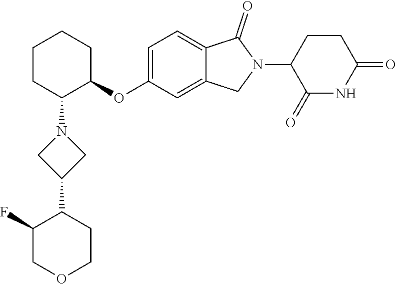 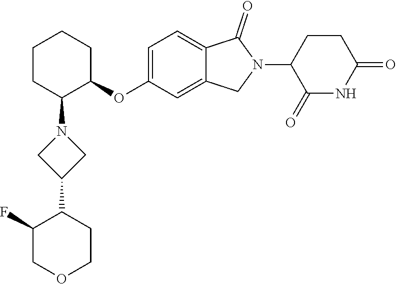 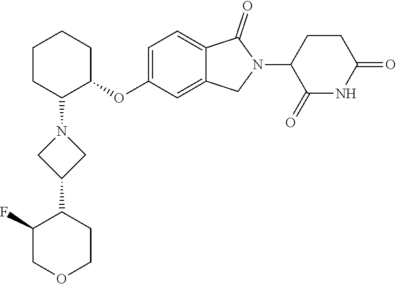 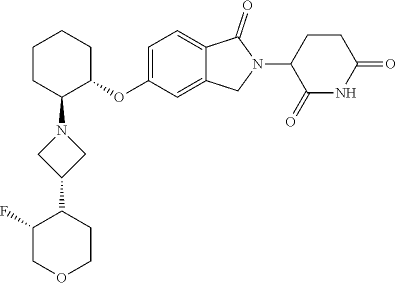 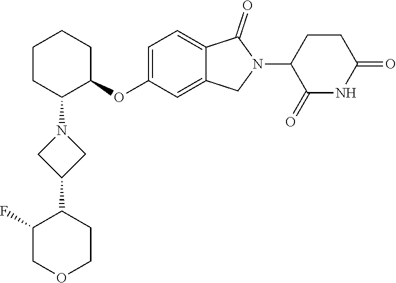 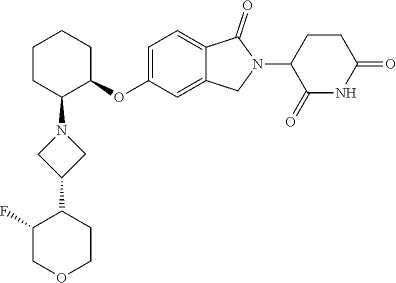 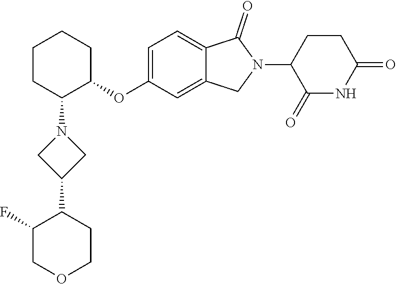 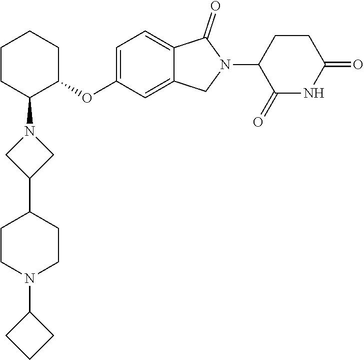 [SMILES: O=C1CCC(N2Cc3cc(O[C@H]4CCCC[C@@H]4N4CC(C5CCN(C6CCC6)CC5)C4)ccc3C2=O)C(=O)N1] 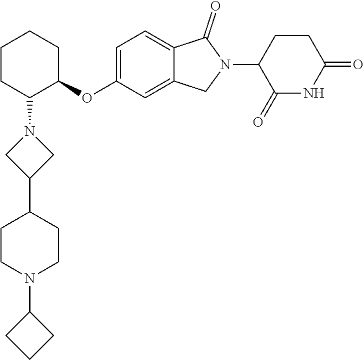 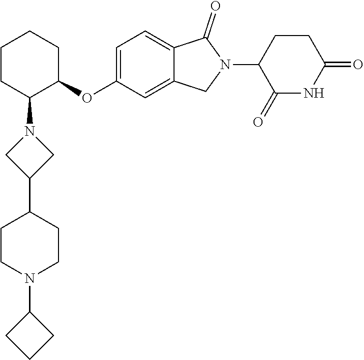 [SMILES: O=C1CCC(N2Cc3cc(O[C@@H]4CCCC[C@@H]4N4CC(C5CCN(C6CCC6)CC5)C4)ccc3C2=O)C(=O)N1] 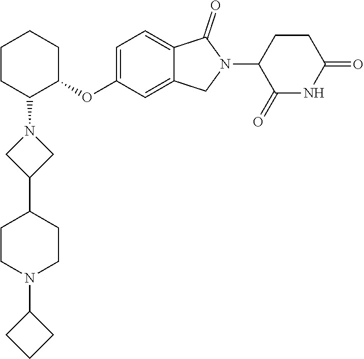 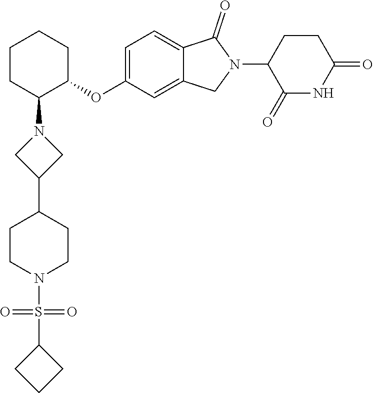 [SMILES: O=C1CCC(N2Cc3cc(O[C@H]4CCCC[C@@H]4N4CC(C5CCN(S(=O)(=O)C6CCC6)CC5)C4)ccc3C2=O)C(=O)N1] 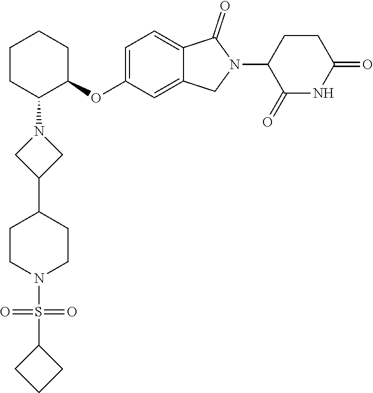 [SMILES: O=C1CCC(N2Cc3cc(O[C@@H]4CCCC[C@H]4N4CC(C5CCN(S(=O)(=O)C6CCC6)CC5)C4)ccc3C2=O)C(=O)N1] 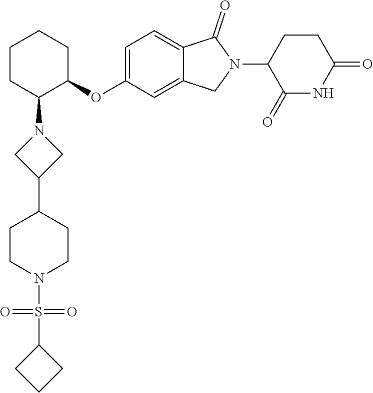 [SMILES: O=C1CCC(N2Cc3cc(O[C@@H]4CCCC[C@@H]4N4CC(C5CCN(S(=O)(=O)C6CCC6)CC5)C4)ccc3C2=O)C(=O)N1] 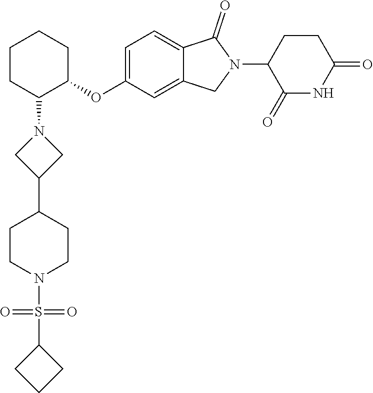 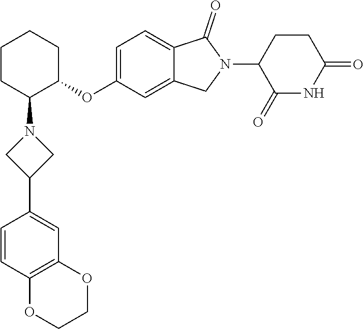 [SMILES: O=C1CCC(N2Cc3cc(O[C@H]4CCCC[C@@H]4N4CC(c5ccc6c(c5)OCCO6)C4)ccc3C2=O)C(=O)N1] 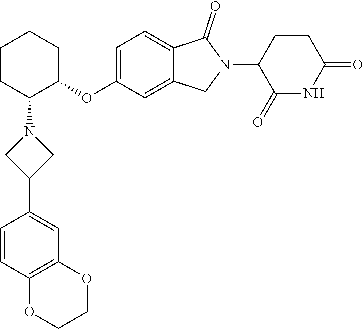 [SMILES: O=C1CCC(N2Cc3cc(O[C@H]4CCCC[C@H]4N4CC(c5ccc6c(c5)OCCO6)C4)ccc3C2=O)C(=O)N1] 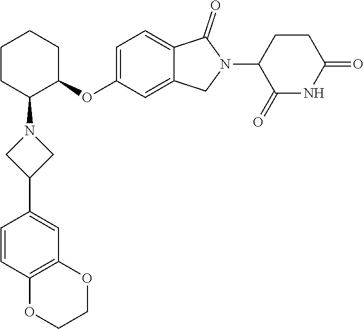 [SMILES: O=C1CCC(N2Cc3cc(O[C@@H]4CCCC[C@@H]4N4CC(c5ccc6c(c5)OCCO6)C4)ccc3C2=O)C(=O)N1] 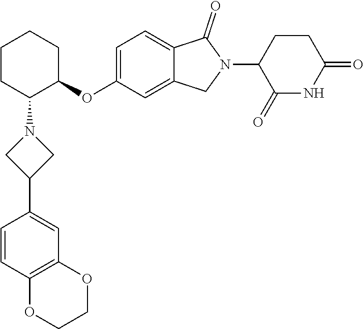 [SMILES: O=C1CCC(N2Cc3cc(O[C@@H]4CCCC[C@H]4N4CC(c5ccc6c(c5)OCCO6)C4)ccc3C2=O)C(=O)N1] 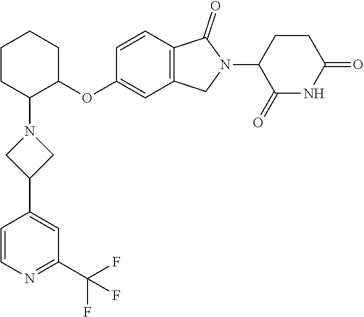 [SMILES: O=C1CCC(N2Cc3cc(OC4CCCCC4N4CC(c5ccnc(C(F)(F)F)c5)C4)ccc3C2=O)C(=O)N1] 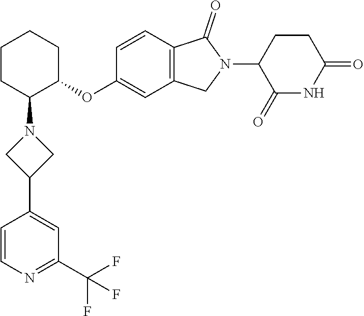 [SMILES: O=C1CCC(N2Cc3cc(O[C@H]4CCCC[C@@H]4N4CC(c5ccnc(C(F)(F)F)c5)C4)ccc3C2=O)C(=O)N1] 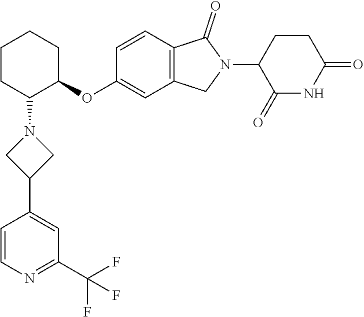 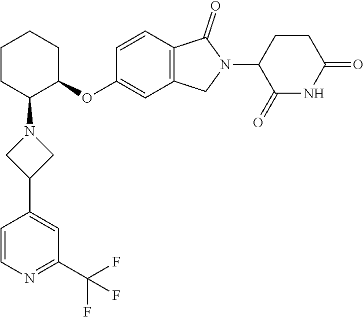 [SMILES: O=C1CCC(N2Cc3cc(O[C@@H]4CCCC[C@@H]4N4CC(c5ccnc(C(F)(F)F)c5)C4)ccc3C2=O)C(=O)N1] 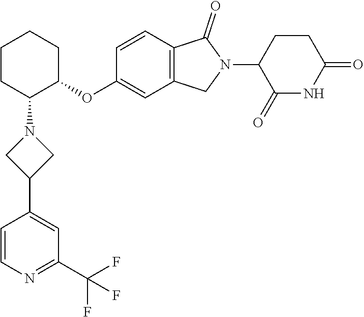 [SMILES: O=C1CCC(N2Cc3cc(O[C@H]4CCCC[C@H]4N4CC(c5ccnc(C(F)(F)F)c5)C4)ccc3C2=O)C(=O)N1] 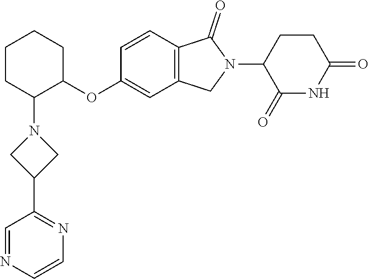 [SMILES: O=C1CCC(N2Cc3cc(OC4CCCCC4N4CC(c5cnccn5)C4)ccc3C2=O)C(=O)N1] 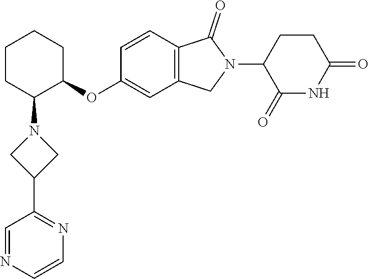 [SMILES: O=C1CCC(N2Cc3cc(O[C@@H]4CCCC[C@@H]4N4CC(c5cnccn5)C4)ccc3C2=O)C(=O)N1] 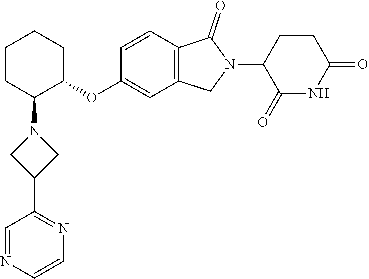 [SMILES: O=C1CCC(N2Cc3cc(O[C@H]4CCCC[C@@H]4N4CC(c5cnccn5)C4)ccc3C2=O)C(=O)N1] 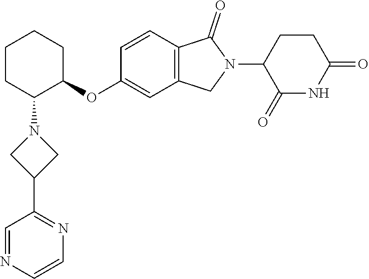 [SMILES: O=C1CCC(N2Cc3cc(O[C@@H]4CCCC[C@H]4N4CC(c5cnccn5)C4)ccc3C2=O)C(=O)N1] 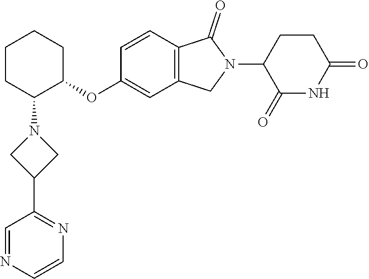 [SMILES: O=C1CCC(N2Cc3cc(O[C@H]4CCCC[C@H]4N4CC(c5cnccn5)C4)ccc3C2=O)C(=O)N1] 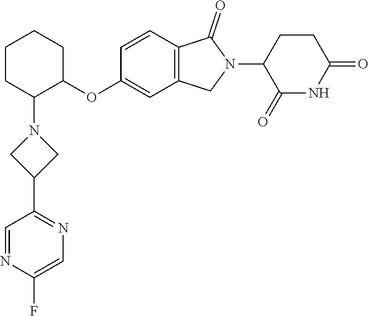 [SMILES: O=C1CCC(N2Cc3cc(OC4CCCCC4N4CC(c5cnc(F)cn5)C4)ccc3C2=O)C(=O)N1] 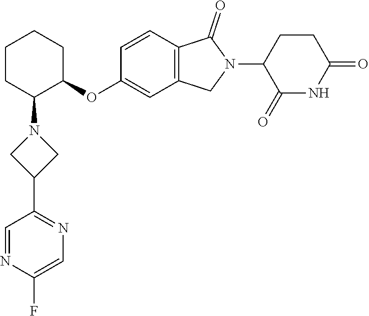 [SMILES: O=C1CCC(N2Cc3cc(O[C@@H]4CCCC[C@@H]4N4CC(c5cnc(F)cn5)C4)ccc3C2=O)C(=O)N1] 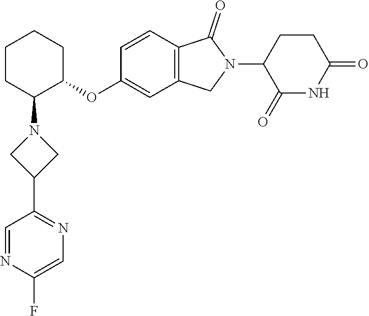 [SMILES: O=C1CCC(N2Cc3cc(O[C@H]4CCCC[C@@H]4N4CC(c5cnc(F)cn5)C4)ccc3C2=O)C(=O)N1] 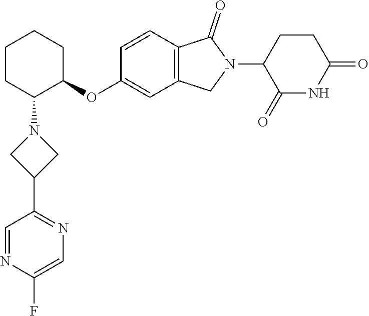 [SMILES: O=C1CCC(N2Cc3cc(O[C@@H]4CCCC[C@H]4N4CC(c5cnc(F)cn5)C4)ccc3C2=O)C(=O)N1] 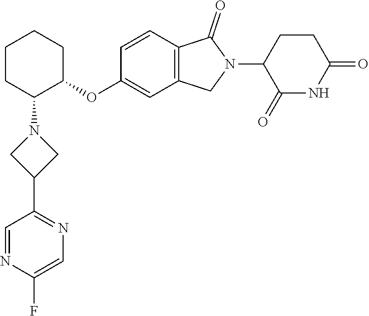 [SMILES: O=C1CCC(N2Cc3cc(O[C@H]4CCCC[C@H]4N4CC(c5cnc(F)cn5)C4)ccc3C2=O)C(=O)N1] 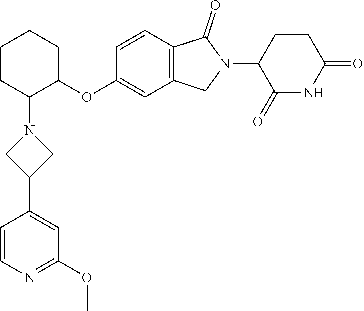 [SMILES: COc1cc(C2CN(C3CCCCC3Oc3ccc4c(c3)CN(C3CCC(=O)NC3=O)C4=O)C2)ccn1] 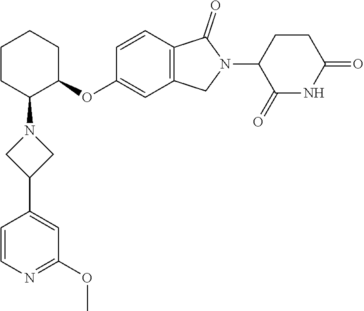 [SMILES: COc1cc(C2CN([C@H]3CCCC[C@H]3Oc3ccc4c(c3)CN(C3CCC(=O)NC3=O)C4=O)C2)ccn1] 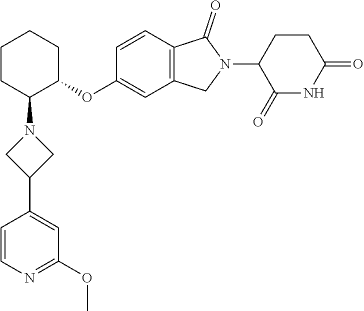 [SMILES: COc1cc(C2CN([C@H]3CCCC[C@@H]3Oc3ccc4c(c3)CN(C3CCC(=O)NC3=O)C4=O)C2)ccn1] 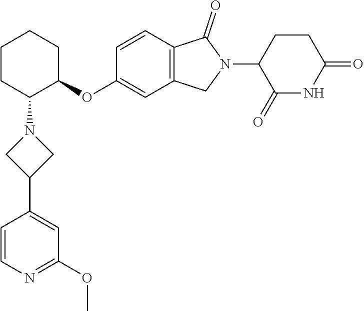 [SMILES: COc1cc(C2CN([C@@H]3CCCC[C@H]3Oc3ccc4c(c3)CN(C3CCC(=O)NC3=O)C4=O)C2)ccn1] 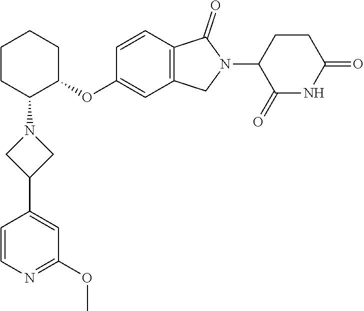 [SMILES: COc1cc(C2CN([C@@H]3CCCC[C@@H]3Oc3ccc4c(c3)CN(C3CCC(=O)NC3=O)C4=O)C2)ccn1] 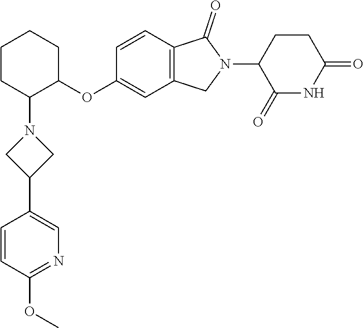 [SMILES: COc1ccc(C2CN(C3CCCCC3Oc3ccc4c(c3)CN(C3CCC(=O)NC3=O)C4=O)C2)cn1] 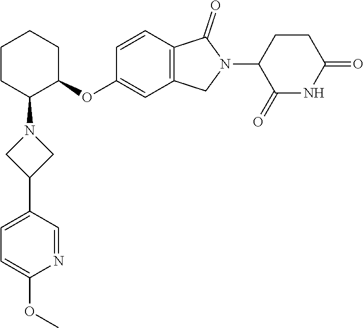 [SMILES: COc1ccc(C2CN([C@H]3CCCC[C@H]3Oc3ccc4c(c3)CN(C3CCC(=O)NC3=O)C4=O)C2)cn1] 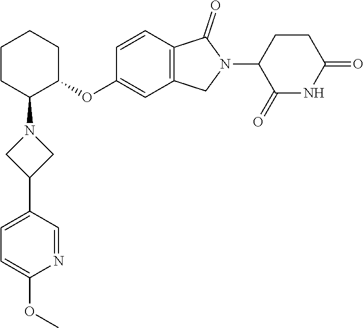 [SMILES: COc1ccc(C2CN([C@H]3CCCC[C@@H]3Oc3ccc4c(c3)CN(C3CCC(=O)NC3=O)C4=O)C2)cn1] 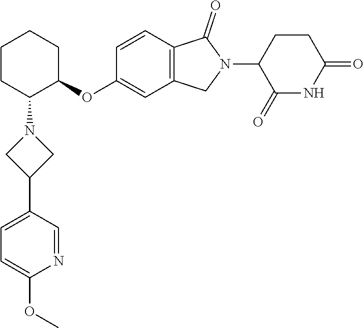 [SMILES: COc1ccc(C2CN([C@@H]3CCCC[C@H]3Oc3ccc4c(c3)CN(C3CCC(=O)NC3=O)C4=O)C2)cn1] 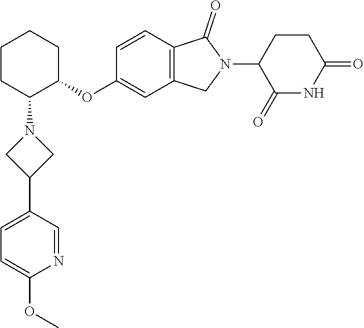 [SMILES: COc1ccc(C2CN([C@@H]3CCCC[C@@H]3Oc3ccc4c(c3)CN(C3CCC(=O)NC3=O)C4=O)C2)cn1] 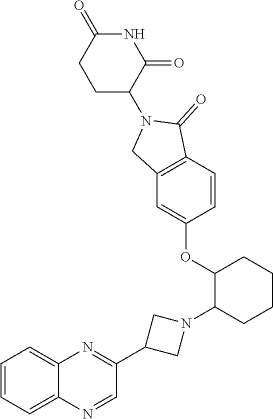 [SMILES: O=C1CCC(N2Cc3cc(OC4CCCCC4N4CC(c5cnc6ccccc6n5)C4)ccc3C2=O)C(=O)N1] 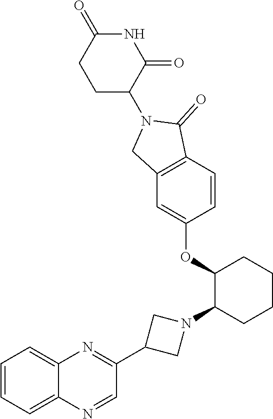 [SMILES: O=C1CCC(N2Cc3cc(O[C@H]4CCCC[C@H]4N4CC(c5cnc6ccccc6n5)C4)ccc3C2=O)C(=O)N1] 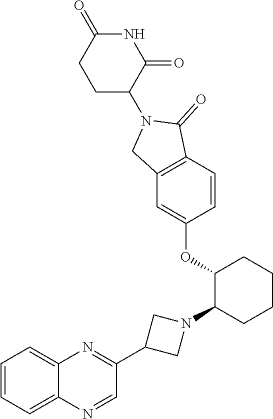 [SMILES: O=C1CCC(N2Cc3cc(O[C@@H]4CCCC[C@H]4N4CC(c5cnc6ccccc6n5)C4)ccc3C2=O)C(=O)N1] 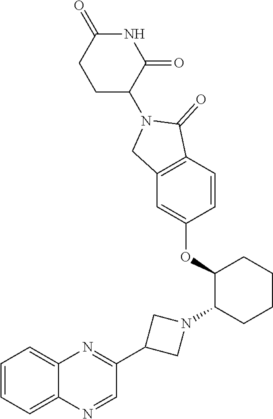 [SMILES: O=C1CCC(N2Cc3cc(O[C@H]4CCCC[C@@H]4N4CC(c5cnc6ccccc6n5)C4)ccc3C2=O)C(=O)N1] 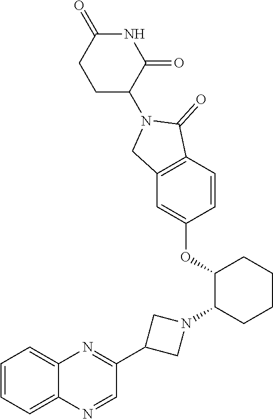 [SMILES: O=C1CCC(N2Cc3cc(O[C@@H]4CCCC[C@@H]4N4CC(c5cnc6ccccc6n5)C4)ccc3C2=O)C(=O)N1] 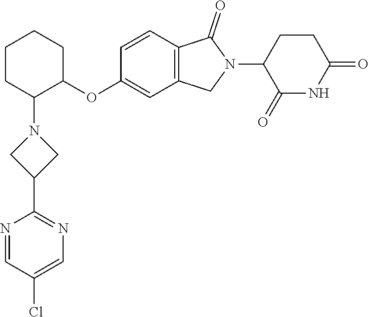 [SMILES: O=C1CCC(N2Cc3cc(OC4CCCCC4N4CC(c5ncc(Cl)cn5)C4)ccc3C2=O)C(=O)N1] 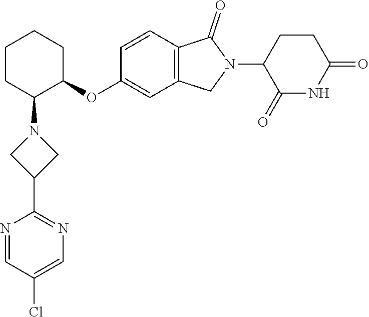 [SMILES: O=C1CCC(N2Cc3cc(O[C@@H]4CCCC[C@@H]4N4CC(c5ncc(Cl)cn5)C4)ccc3C2=O)C(=O)N1] 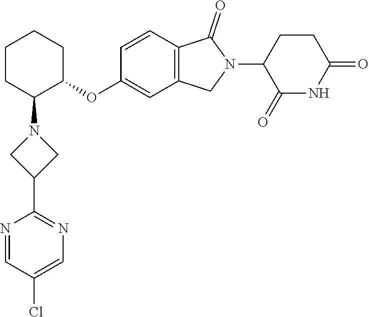 [SMILES: O=C1CCC(N2Cc3cc(O[C@H]4CCCC[C@@H]4N4CC(c5ncc(Cl)cn5)C4)ccc3C2=O)C(=O)N1] 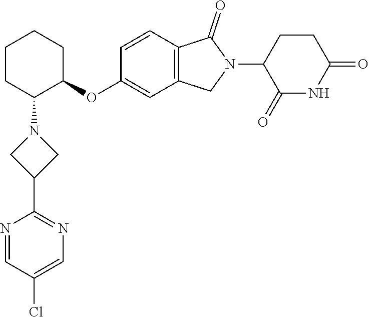 [SMILES: O=C1CCC(N2Cc3cc(O[C@@H]4CCCC[C@H]4N4CC(c5ncc(Cl)cn5)C4)ccc3C2=O)C(=O)N1] 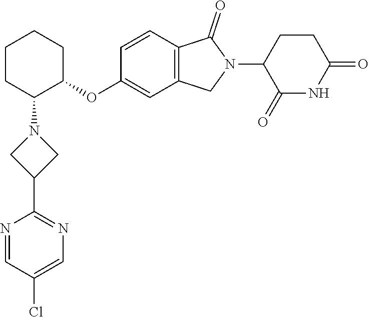 [SMILES: O=C1CCC(N2Cc3cc(O[C@H]4CCCC[C@H]4N4CC(c5ncc(Cl)cn5)C4)ccc3C2=O)C(=O)N1] 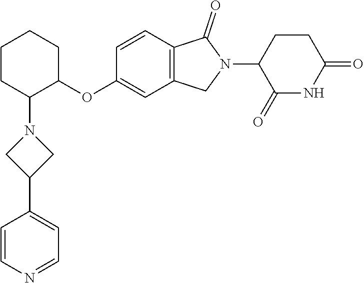 [SMILES: O=C1CCC(N2Cc3cc(OC4CCCCC4N4CC(c5ccncc5)C4)ccc3C2=O)C(=O)N1] 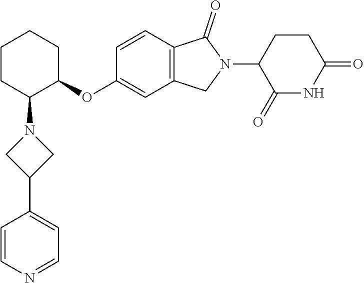 [SMILES: O=C1CCC(N2Cc3cc(O[C@@H]4CCCC[C@@H]4N4CC(c5ccncc5)C4)ccc3C2=O)C(=O)N1] 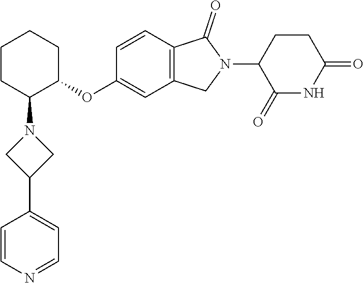 [SMILES: O=C1CCC(N2Cc3cc(O[C@H]4CCCC[C@@H]4N4CC(c5ccncc5)C4)ccc3C2=O)C(=O)N1] 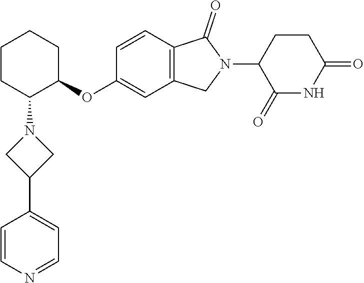 [SMILES: O=C1CCC(N2Cc3cc(O[C@@H]4CCCC[C@H]4N4CC(c5ccncc5)C4)ccc3C2=O)C(=O)N1] 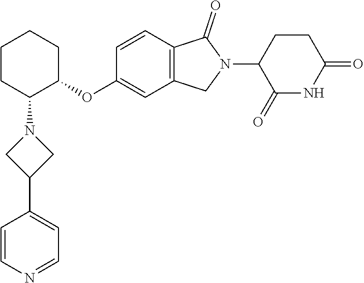 [SMILES: O=C1CCC(N2Cc3cc(O[C@H]4CCCC[C@H]4N4CC(c5ccncc5)C4)ccc3C2=O)C(=O)N1] 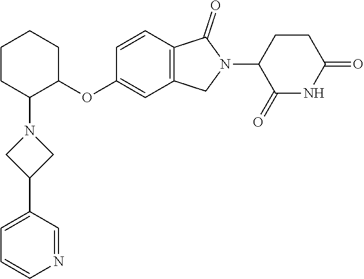 [SMILES: O=C1CCC(N2Cc3cc(OC4CCCCC4N4CC(c5cccnc5)C4)ccc3C2=O)C(=O)N1] 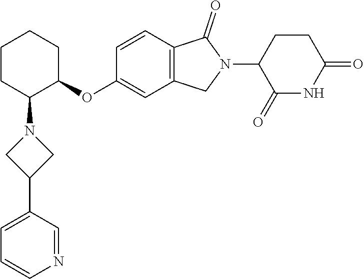 [SMILES: O=C1CCC(N2Cc3cc(O[C@@H]4CCCC[C@@H]4N4CC(c5cccnc5)C4)ccc3C2=O)C(=O)N1] 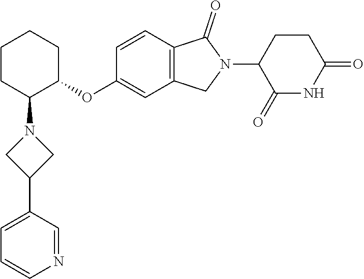 [SMILES: O=C1CCC(N2Cc3cc(O[C@H]4CCCC[C@@H]4N4CC(c5cccnc5)C4)ccc3C2=O)C(=O)N1] 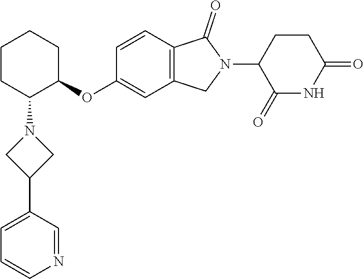 [SMILES: O=C1CCC(N2Cc3cc(O[C@@H]4CCCC[C@H]4N4CC(c5cccnc5)C4)ccc3C2=O)C(=O)N1] 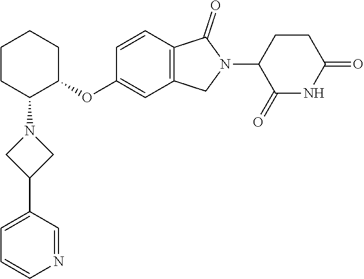 [SMILES: O=C1CCC(N2Cc3cc(O[C@H]4CCCC[C@H]4N4CC(c5cccnc5)C4)ccc3C2=O)C(=O)N1] 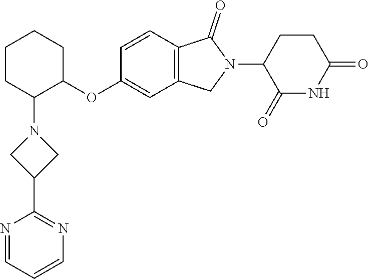 [SMILES: O=C1CCC(N2Cc3cc(OC4CCCCC4N4CC(c5ncccn5)C4)ccc3C2=O)C(=O)N1] 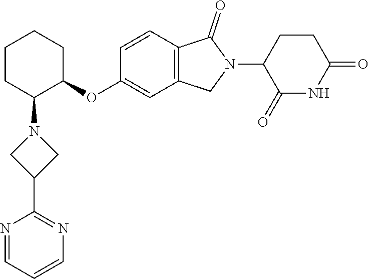 [SMILES: O=C1CCC(N2Cc3cc(O[C@@H]4CCCC[C@@H]4N4CC(c5ncccn5)C4)ccc3C2=O)C(=O)N1] 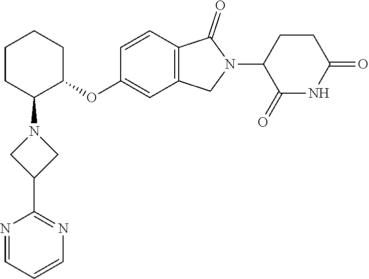 [SMILES: O=C1CCC(N2Cc3cc(O[C@H]4CCCC[C@@H]4N4CC(c5ncccn5)C4)ccc3C2=O)C(=O)N1] 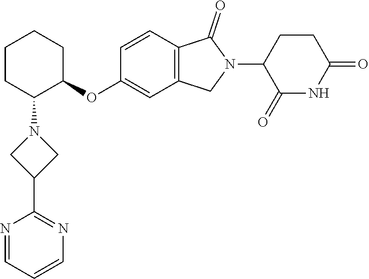 [SMILES: O=C1CCC(N2Cc3cc(O[C@@H]4CCCC[C@H]4N4CC(c5ncccn5)C4)ccc3C2=O)C(=O)N1] 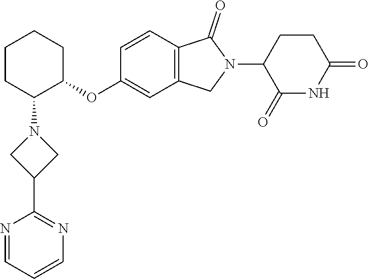 [SMILES: O=C1CCC(N2Cc3cc(O[C@H]4CCCC[C@H]4N4CC(c5ncccn5)C4)ccc3C2=O)C(=O)N1] 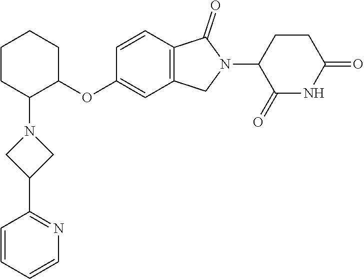 [SMILES: O=C1CCC(N2Cc3cc(OC4CCCCC4N4CC(c5ccccn5)C4)ccc3C2=O)C(=O)N1] 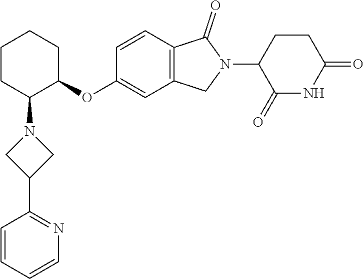 [SMILES: O=C1CCC(N2Cc3cc(O[C@@H]4CCCC[C@@H]4N4CC(c5ccccn5)C4)ccc3C2=O)C(=O)N1] 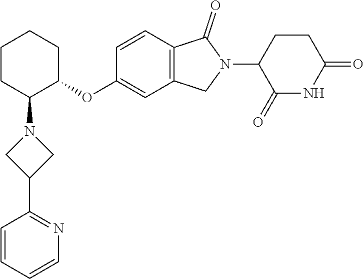 [SMILES: O=C1CCC(N2Cc3cc(O[C@H]4CCCC[C@@H]4N4CC(c5ccccn5)C4)ccc3C2=O)C(=O)N1] 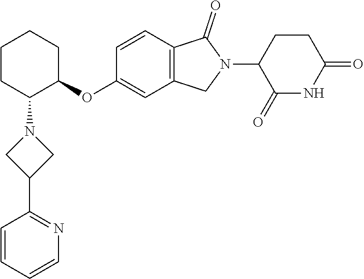 [SMILES: O=C1CCC(N2Cc3cc(O[C@@H]4CCCC[C@H]4N4CC(c5ccccn5)C4)ccc3C2=O)C(=O)N1] 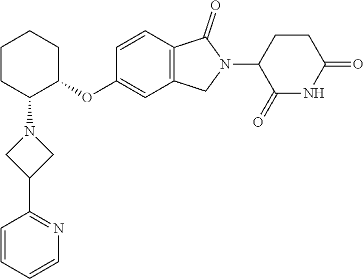 [SMILES: O=C1CCC(N2Cc3cc(O[C@H]4CCCC[C@H]4N4CC(c5ccccn5)C4)ccc3C2=O)C(=O)N1] 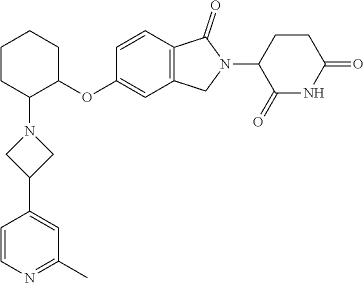 [SMILES: Cc1cc(C2CN(C3CCCCC3Oc3ccc4c(c3)CN(C3CCC(=O)NC3=O)C4=O)C2)ccn1] 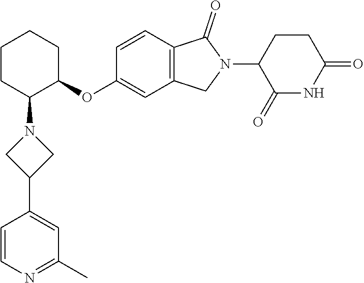 [SMILES: Cc1cc(C2CN([C@H]3CCCC[C@H]3Oc3ccc4c(c3)CN(C3CCC(=O)NC3=O)C4=O)C2)ccn1] 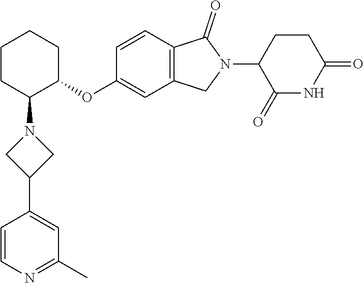 [SMILES: Cc1cc(C2CN([C@H]3CCCC[C@@H]3Oc3ccc4c(c3)CN(C3CCC(=O)NC3=O)C4=O)C2)ccn1] 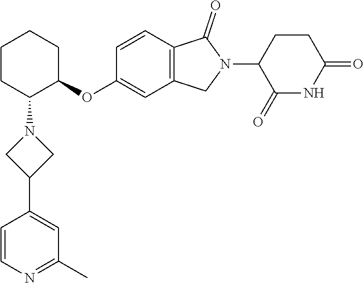 [SMILES: Cc1cc(C2CN([C@@H]3CCCC[C@H]3Oc3ccc4c(c3)CN(C3CCC(=O)NC3=O)C4=O)C2)ccn1] 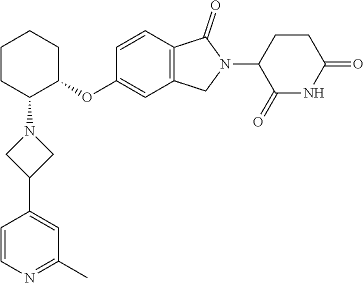 [SMILES: Cc1cc(C2CN([C@@H]3CCCC[C@@H]3Oc3ccc4c(c3)CN(C3CCC(=O)NC3=O)C4=O)C2)ccn1] 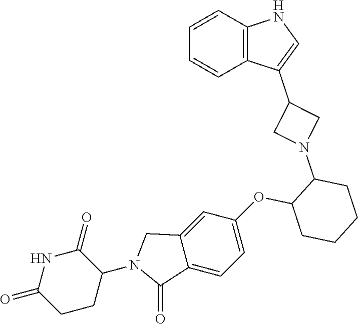 [SMILES: O=C1CCC(N2Cc3cc(OC4CCCCC4N4CC(c5c[nH]c6ccccc56)C4)ccc3C2=O)C(=O)N1] 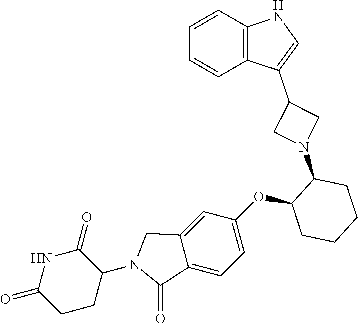 [SMILES: O=C1CCC(N2Cc3cc(O[C@@H]4CCCC[C@@H]4N4CC(c5c[nH]c6ccccc56)C4)ccc3C2=O)C(=O)N1] 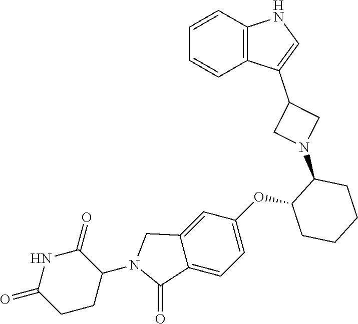 [SMILES: O=C1CCC(N2Cc3cc(O[C@H]4CCCC[C@@H]4N4CC(c5c[nH]c6ccccc56)C4)ccc3C2=O)C(=O)N1] 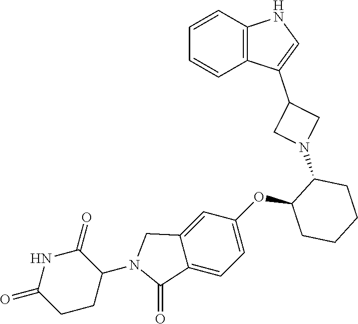 [SMILES: O=C1CCC(N2Cc3cc(O[C@@H]4CCCC[C@H]4N4CC(c5c[nH]c6ccccc56)C4)ccc3C2=O)C(=O)N1] 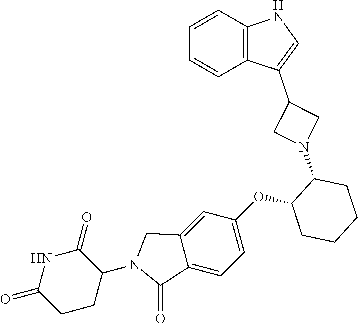 [SMILES: O=C1CCC(N2Cc3cc(O[C@H]4CCCC[C@H]4N4CC(c5c[nH]c6ccccc56)C4)ccc3C2=O)C(=O)N1] 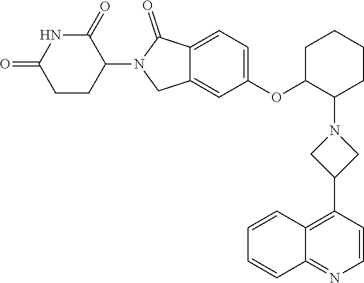 [SMILES: O=C1CCC(N2Cc3cc(OC4CCCCC4N4CC(c5ccnc6ccccc56)C4)ccc3C2=O)C(=O)N1] 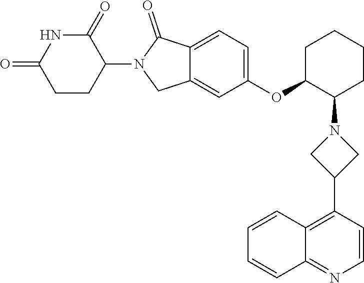 [SMILES: O=C1CCC(N2Cc3cc(O[C@H]4CCCC[C@H]4N4CC(c5ccnc6ccccc56)C4)ccc3C2=O)C(=O)N1] 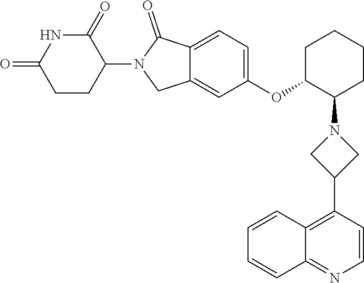 [SMILES: O=C1CCC(N2Cc3cc(O[C@@H]4CCCC[C@H]4N4CC(c5ccnc6ccccc56)C4)ccc3C2=O)C(=O)N1] 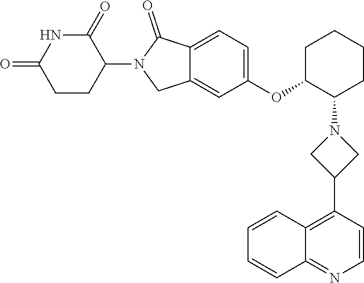 [SMILES: O=C1CCC(N2Cc3cc(O[C@@H]4CCCC[C@@H]4N4CC(c5ccnc6ccccc56)C4)ccc3C2=O)C(=O)N1] 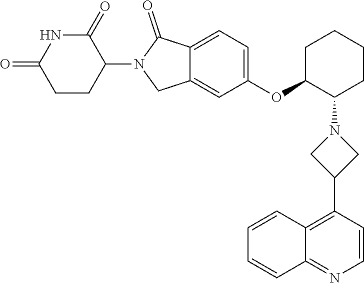 [SMILES: O=C1CCC(N2Cc3cc(O[C@H]4CCCC[C@@H]4N4CC(c5ccnc6ccccc56)C4)ccc3C2=O)C(=O)N1] 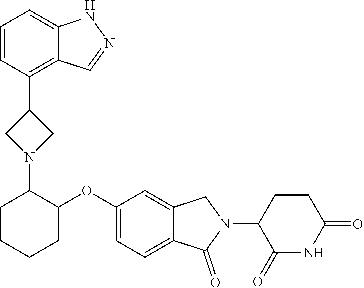 [SMILES: O=C1CCC(N2Cc3cc(OC4CCCCC4N4CC(c5cccc6[nH]ncc56)C4)ccc3C2=O)C(=O)N1] 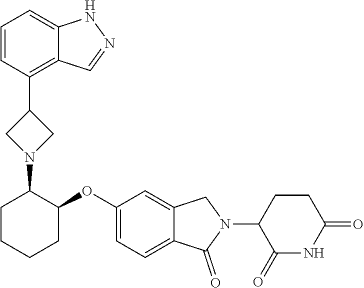 [SMILES: O=C1CCC(N2Cc3cc(O[C@H]4CCCC[C@H]4N4CC(c5cccc6[nH]ncc56)C4)ccc3C2=O)C(=O)N1] 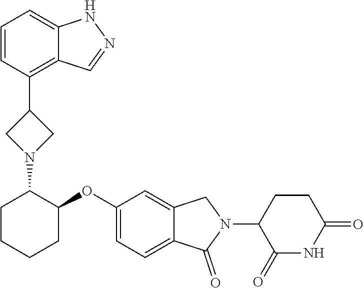 [SMILES: O=C1CCC(N2Cc3cc(O[C@H]4CCCC[C@@H]4N4CC(c5cccc6[nH]ncc56)C4)ccc3C2=O)C(=O)N1] 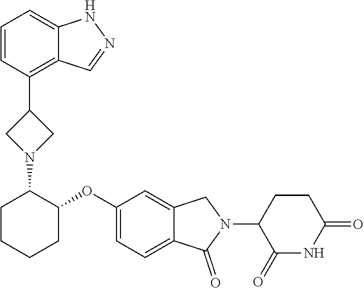 [SMILES: O=C1CCC(N2Cc3cc(O[C@@H]4CCCC[C@@H]4N4CC(c5cccc6[nH]ncc56)C4)ccc3C2=O)C(=O)N1] 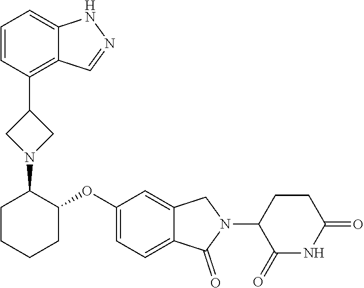 [SMILES: O=C1CCC(N2Cc3cc(O[C@@H]4CCCC[C@H]4N4CC(c5cccc6[nH]ncc56)C4)ccc3C2=O)C(=O)N1] 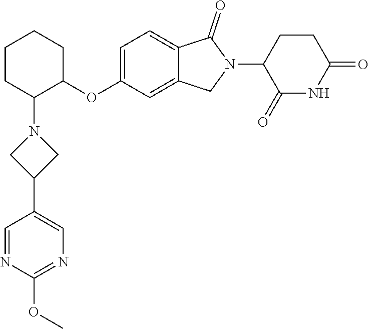 [SMILES: COc1ncc(C2CN(C3CCCCC3Oc3ccc4c(c3)CN(C3CCC(=O)NC3=O)C4=O)C2)cn1] 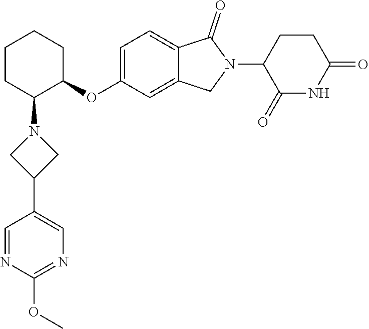 [SMILES: COc1ncc(C2CN([C@H]3CCCC[C@H]3Oc3ccc4c(c3)CN(C3CCC(=O)NC3=O)C4=O)C2)cn1] 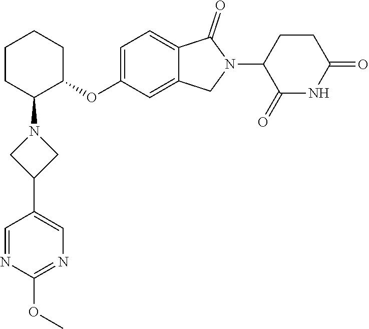 [SMILES: COc1ncc(C2CN([C@H]3CCCC[C@@H]3Oc3ccc4c(c3)CN(C3CCC(=O)NC3=O)C4=O)C2)cn1] 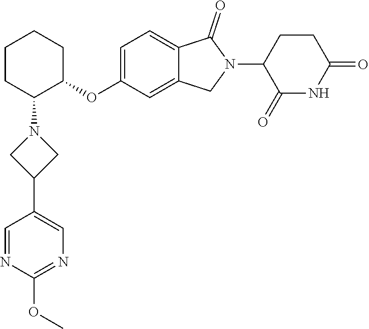 [SMILES: COc1ncc(C2CN([C@@H]3CCCC[C@@H]3Oc3ccc4c(c3)CN(C3CCC(=O)NC3=O)C4=O)C2)cn1] 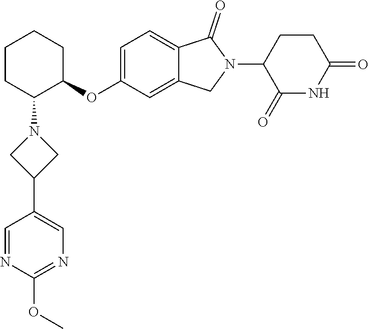 [SMILES: COc1ncc(C2CN([C@@H]3CCCC[C@H]3Oc3ccc4c(c3)CN(C3CCC(=O)NC3=O)C4=O)C2)cn1] 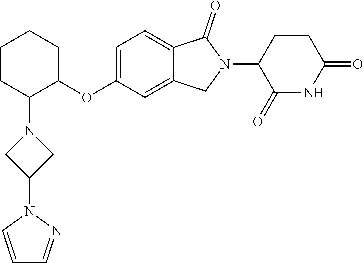 [SMILES: O=C1CCC(N2Cc3cc(OC4CCCCC4N4CC(n5cccn5)C4)ccc3C2=O)C(=O)N1] 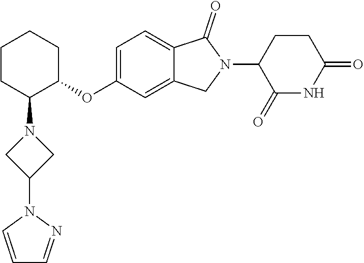 [SMILES: O=C1CCC(N2Cc3cc(O[C@H]4CCCC[C@@H]4N4CC(n5cccn5)C4)ccc3C2=O)C(=O)N1] 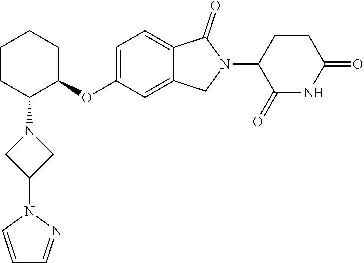 [SMILES: O=C1CCC(N2Cc3cc(O[C@@H]4CCCC[C@H]4N4CC(n5cccn5)C4)ccc3C2=O)C(=O)N1] 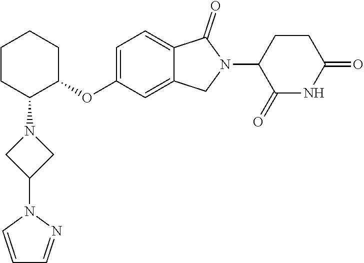 [SMILES: O=C1CCC(N2Cc3cc(O[C@H]4CCCC[C@H]4N4CC(n5cccn5)C4)ccc3C2=O)C(=O)N1] 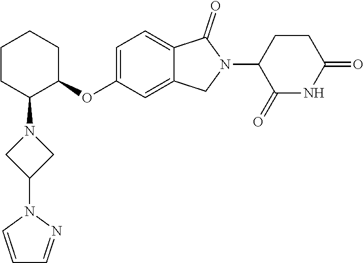 [SMILES: O=C1CCC(N2Cc3cc(O[C@@H]4CCCC[C@@H]4N4CC(n5cccn5)C4)ccc3C2=O)C(=O)N1] 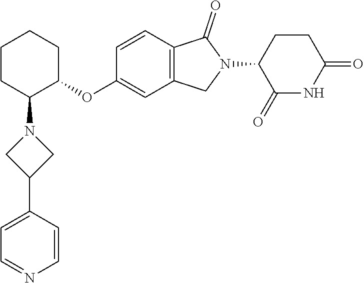 [SMILES: O=C1CC[C@@H](N2Cc3cc(O[C@H]4CCCC[C@@H]4N4CC(c5ccncc5)C4)ccc3C2=O)C(=O)N1] 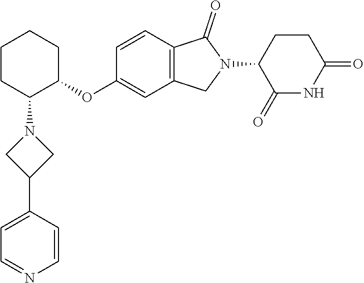 [SMILES: O=C1CC[C@@H](N2Cc3cc(O[C@H]4CCCC[C@H]4N4CC(c5ccncc5)C4)ccc3C2=O)C(=O)N1] 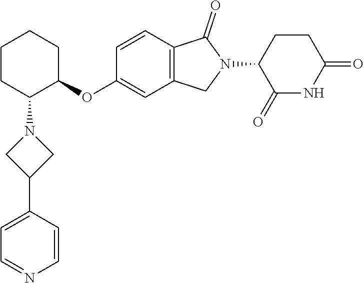 [SMILES: O=C1CC[C@@H](N2Cc3cc(O[C@@H]4CCCC[C@H]4N4CC(c5ccncc5)C4)ccc3C2=O)C(=O)N1] 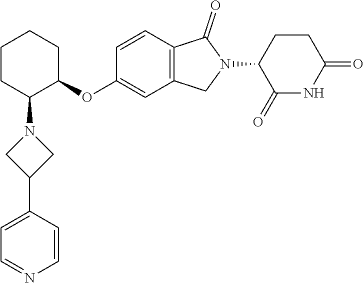 [SMILES: O=C1CC[C@@H](N2Cc3cc(O[C@@H]4CCCC[C@@H]4N4CC(c5ccncc5)C4)ccc3C2=O)C(=O)N1] 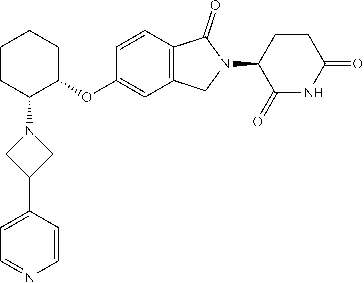 [SMILES: O=C1CC[C@H](N2Cc3cc(O[C@H]4CCCC[C@H]4N4CC(c5ccncc5)C4)ccc3C2=O)C(=O)N1] 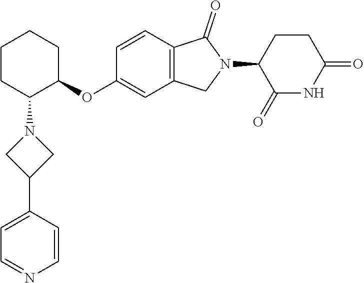 [SMILES: O=C1CC[C@H](N2Cc3cc(O[C@@H]4CCCC[C@H]4N4CC(c5ccncc5)C4)ccc3C2=O)C(=O)N1] 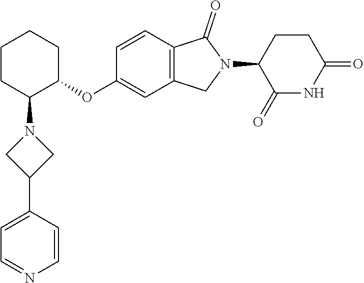 [SMILES: O=C1CC[C@H](N2Cc3cc(O[C@H]4CCCC[C@@H]4N4CC(c5ccncc5)C4)ccc3C2=O)C(=O)N1] 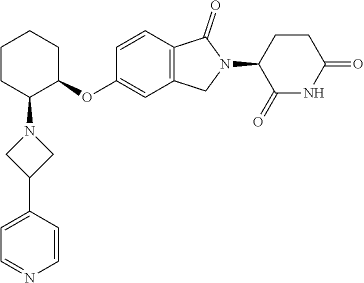 [SMILES: O=C1CC[C@H](N2Cc3cc(O[C@@H]4CCCC[C@@H]4N4CC(c5ccncc5)C4)ccc3C2=O)C(=O)N1] 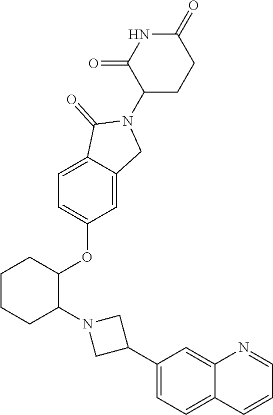 [SMILES: O=C1CCC(N2Cc3cc(OC4CCCCC4N4CC(c5ccc6cccnc6c5)C4)ccc3C2=O)C(=O)N1] 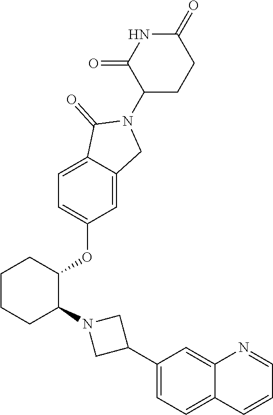 [SMILES: O=C1CCC(N2Cc3cc(O[C@H]4CCCC[C@@H]4N4CC(c5ccc6cccnc6c5)C4)ccc3C2=O)C(=O)N1] 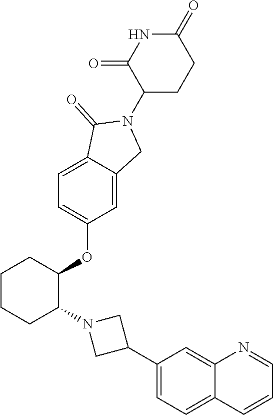 [SMILES: O=C1CCC(N2Cc3cc(O[C@@H]4CCCC[C@H]4N4CC(c5ccc6cccnc6c5)C4)ccc3C2=O)C(=O)N1] 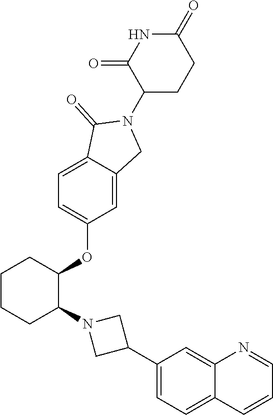 [SMILES: O=C1CCC(N2Cc3cc(O[C@@H]4CCCC[C@@H]4N4CC(c5ccc6cccnc6c5)C4)ccc3C2=O)C(=O)N1] 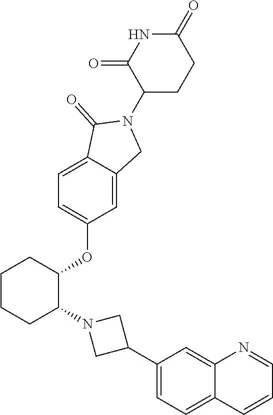 [SMILES: O=C1CCC(N2Cc3cc(O[C@H]4CCCC[C@H]4N4CC(c5ccc6cccnc6c5)C4)ccc3C2=O)C(=O)N1] 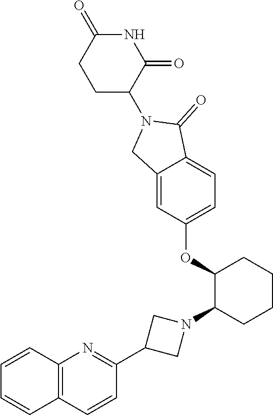 [SMILES: O=C1CCC(N2Cc3cc(O[C@H]4CCCC[C@H]4N4CC(c5ccc6ccccc6n5)C4)ccc3C2=O)C(=O)N1] 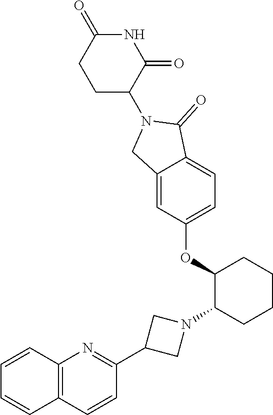 [SMILES: O=C1CCC(N2Cc3cc(O[C@H]4CCCC[C@@H]4N4CC(c5ccc6ccccc6n5)C4)ccc3C2=O)C(=O)N1] 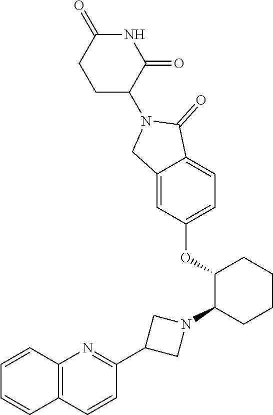 [SMILES: O=C1CCC(N2Cc3cc(O[C@@H]4CCCC[C@H]4N4CC(c5ccc6ccccc6n5)C4)ccc3C2=O)C(=O)N1] 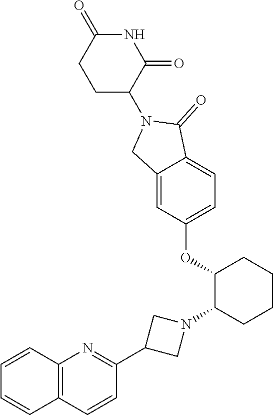 [SMILES: O=C1CCC(N2Cc3cc(O[C@@H]4CCCC[C@@H]4N4CC(c5ccc6ccccc6n5)C4)ccc3C2=O)C(=O)N1] 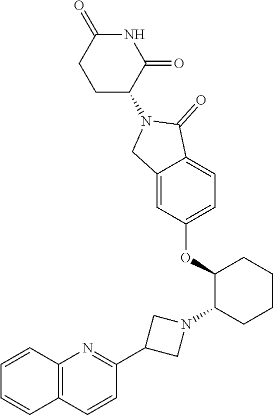 [SMILES: O=C1CC[C@@H](N2Cc3cc(O[C@H]4CCCC[C@@H]4N4CC(c5ccc6ccccc6n5)C4)ccc3C2=O)C(=O)N1] 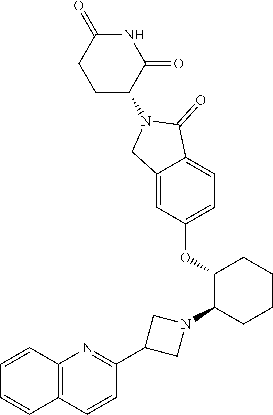 [SMILES: O=C1CC[C@@H](N2Cc3cc(O[C@@H]4CCCC[C@H]4N4CC(c5ccc6ccccc6n5)C4)ccc3C2=O)C(=O)N1] 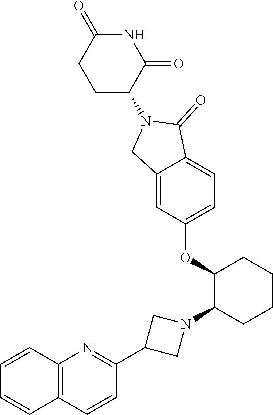 [SMILES: O=C1CC[C@@H](N2Cc3cc(O[C@H]4CCCC[C@H]4N4CC(c5ccc6ccccc6n5)C4)ccc3C2=O)C(=O)N1] 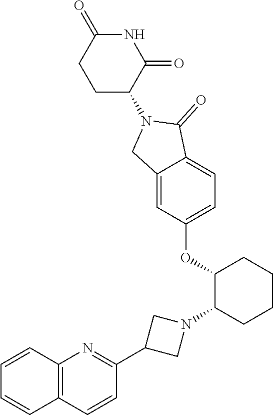 [SMILES: O=C1CC[C@@H](N2Cc3cc(O[C@@H]4CCCC[C@@H]4N4CC(c5ccc6ccccc6n5)C4)ccc3C2=O)C(=O)N1] 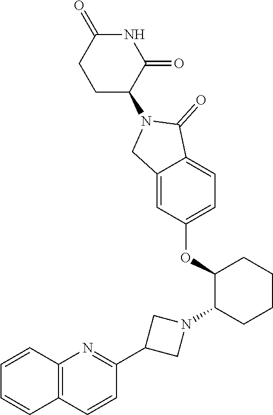 [SMILES: O=C1CC[C@H](N2Cc3cc(O[C@H]4CCCC[C@@H]4N4CC(c5ccc6ccccc6n5)C4)ccc3C2=O)C(=O)N1] 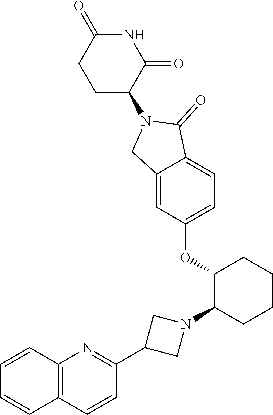 [SMILES: O=C1CC[C@H](N2Cc3cc(O[C@@H]4CCCC[C@H]4N4CC(c5ccc6ccccc6n5)C4)ccc3C2=O)C(=O)N1] 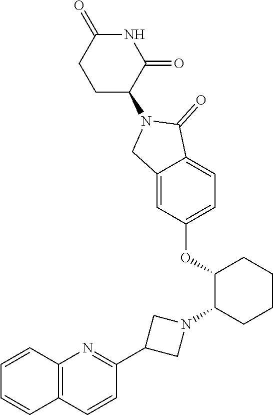 [SMILES: O=C1CC[C@H](N2Cc3cc(O[C@@H]4CCCC[C@@H]4N4CC(c5ccc6ccccc6n5)C4)ccc3C2=O)C(=O)N1] 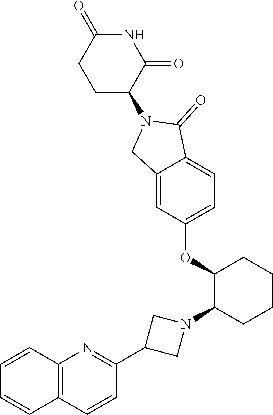 [SMILES: O=C1CC[C@H](N2Cc3cc(O[C@H]4CCCC[C@H]4N4CC(c5ccc6ccccc6n5)C4)ccc3C2=O)C(=O)N1] 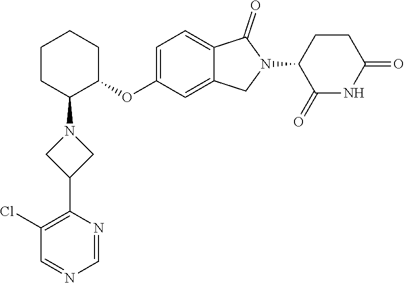 [SMILES: O=C1CC[C@@H](N2Cc3cc(O[C@H]4CCCC[C@@H]4N4CC(c5ncncc5Cl)C4)ccc3C2=O)C(=O)N1] 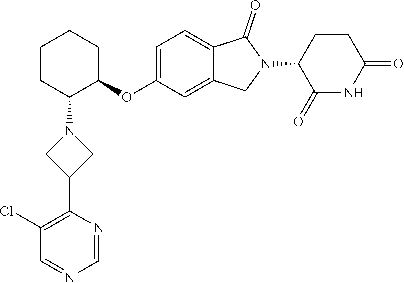 [SMILES: O=C1CC[C@@H](N2Cc3cc(O[C@@H]4CCCC[C@H]4N4CC(c5ncncc5Cl)C4)ccc3C2=O)C(=O)N1] 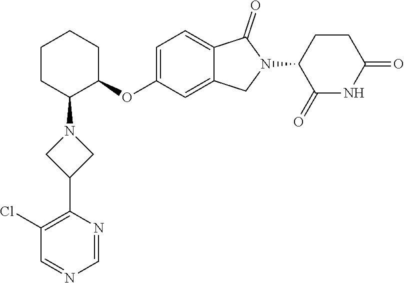 [SMILES: O=C1CC[C@@H](N2Cc3cc(O[C@@H]4CCCC[C@@H]4N4CC(c5ncncc5Cl)C4)ccc3C2=O)C(=O)N1] 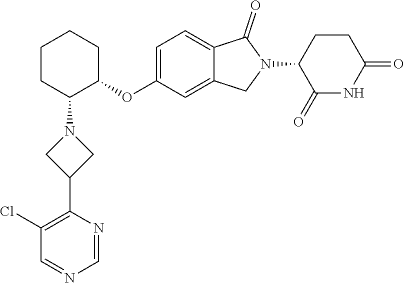 [SMILES: O=C1CC[C@@H](N2Cc3cc(O[C@H]4CCCC[C@H]4N4CC(c5ncncc5Cl)C4)ccc3C2=O)C(=O)N1] 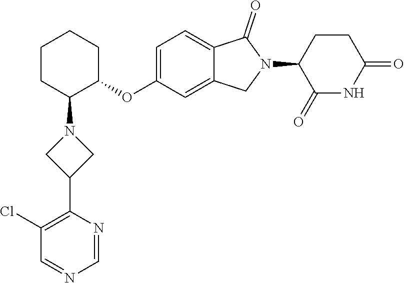 [SMILES: O=C1CC[C@H](N2Cc3cc(O[C@H]4CCCC[C@@H]4N4CC(c5ncncc5Cl)C4)ccc3C2=O)C(=O)N1] 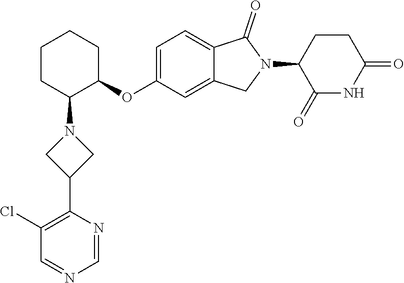 [SMILES: O=C1CC[C@H](N2Cc3cc(O[C@@H]4CCCC[C@@H]4N4CC(c5ncncc5Cl)C4)ccc3C2=O)C(=O)N1] 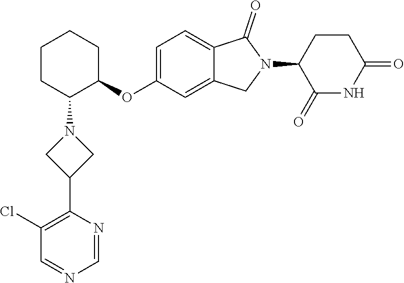 [SMILES: O=C1CC[C@H](N2Cc3cc(O[C@@H]4CCCC[C@H]4N4CC(c5ncncc5Cl)C4)ccc3C2=O)C(=O)N1] 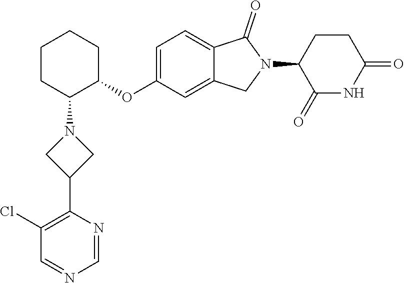 [SMILES: O=C1CC[C@H](N2Cc3cc(O[C@H]4CCCC[C@H]4N4CC(c5ncncc5Cl)C4)ccc3C2=O)C(=O)N1] 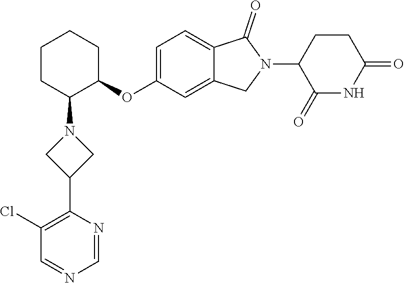 [SMILES: O=C1CCC(N2Cc3cc(O[C@@H]4CCCC[C@@H]4N4CC(c5ncncc5Cl)C4)ccc3C2=O)C(=O)N1] 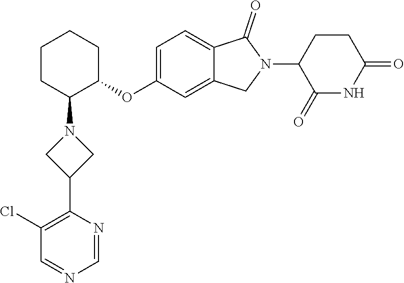 [SMILES: O=C1CCC(N2Cc3cc(O[C@H]4CCCC[C@@H]4N4CC(c5ncncc5Cl)C4)ccc3C2=O)C(=O)N1] 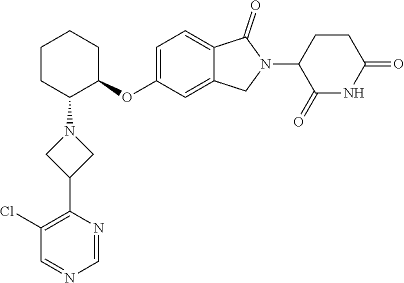 [SMILES: O=C1CCC(N2Cc3cc(O[C@@H]4CCCC[C@H]4N4CC(c5ncncc5Cl)C4)ccc3C2=O)C(=O)N1] 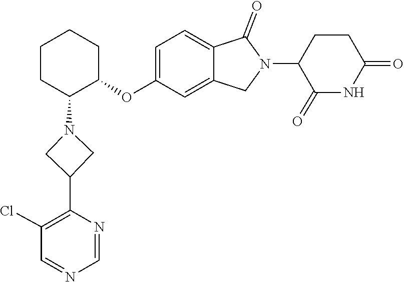 [SMILES: O=C1CCC(N2Cc3cc(O[C@H]4CCCC[C@H]4N4CC(c5ncncc5Cl)C4)ccc3C2=O)C(=O)N1] 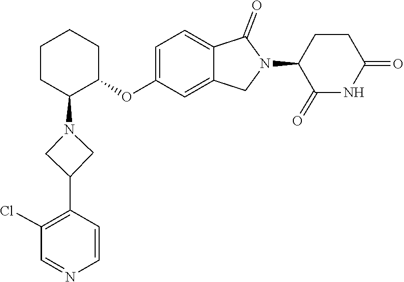 [SMILES: O=C1CC[C@H](N2Cc3cc(O[C@H]4CCCC[C@@H]4N4CC(c5ccncc5Cl)C4)ccc3C2=O)C(=O)N1] 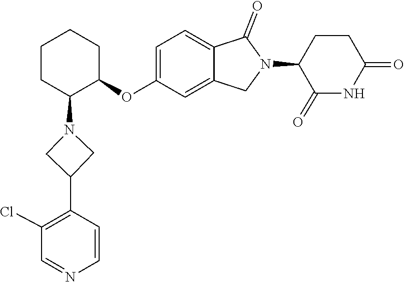 [SMILES: O=C1CC[C@H](N2Cc3cc(O[C@@H]4CCCC[C@@H]4N4CC(c5ccncc5Cl)C4)ccc3C2=O)C(=O)N1] 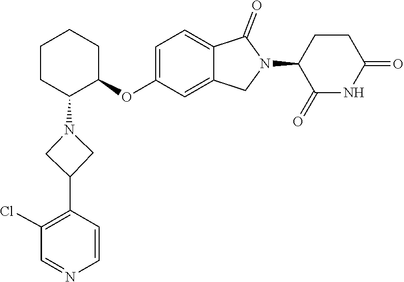 [SMILES: O=C1CC[C@H](N2Cc3cc(O[C@@H]4CCCC[C@H]4N4CC(c5ccncc5Cl)C4)ccc3C2=O)C(=O)N1] 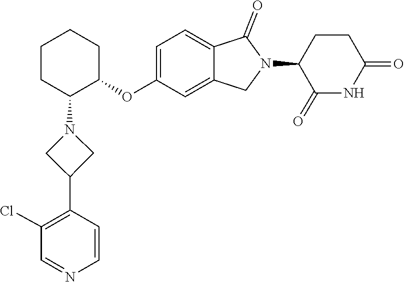 [SMILES: O=C1CC[C@H](N2Cc3cc(O[C@H]4CCCC[C@H]4N4CC(c5ccncc5Cl)C4)ccc3C2=O)C(=O)N1] 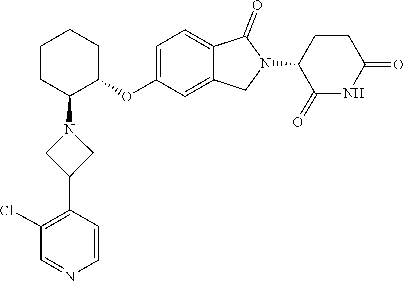 [SMILES: O=C1CC[C@@H](N2Cc3cc(O[C@H]4CCCC[C@@H]4N4CC(c5ccncc5Cl)C4)ccc3C2=O)C(=O)N1] 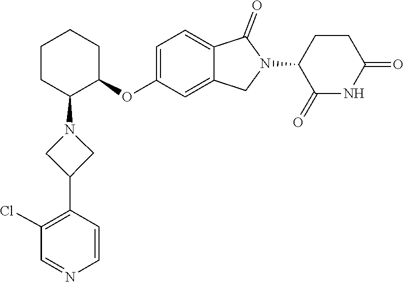 [SMILES: O=C1CC[C@@H](N2Cc3cc(O[C@@H]4CCCC[C@@H]4N4CC(c5ccncc5Cl)C4)ccc3C2=O)C(=O)N1] 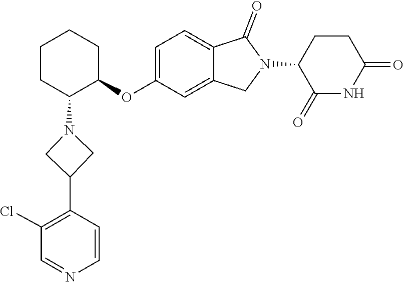 [SMILES: O=C1CC[C@@H](N2Cc3cc(O[C@@H]4CCCC[C@H]4N4CC(c5ccncc5Cl)C4)ccc3C2=O)C(=O)N1] 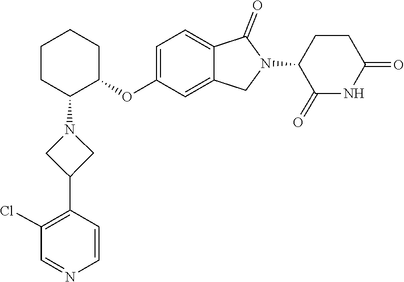 [SMILES: O=C1CC[C@@H](N2Cc3cc(O[C@H]4CCCC[C@H]4N4CC(c5ccncc5Cl)C4)ccc3C2=O)C(=O)N1] 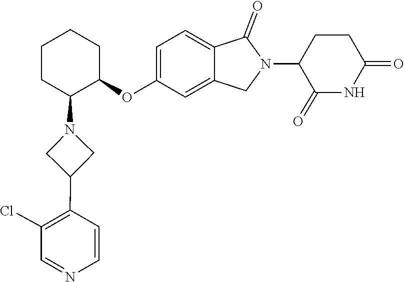 [SMILES: O=C1CCC(N2Cc3cc(O[C@@H]4CCCC[C@@H]4N4CC(c5ccncc5Cl)C4)ccc3C2=O)C(=O)N1] 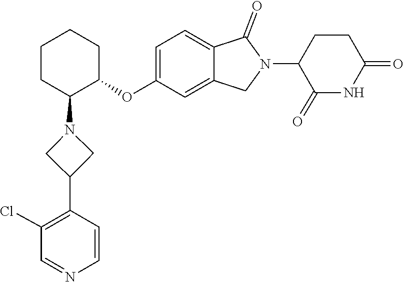 [SMILES: O=C1CCC(N2Cc3cc(O[C@H]4CCCC[C@@H]4N4CC(c5ccncc5Cl)C4)ccc3C2=O)C(=O)N1] 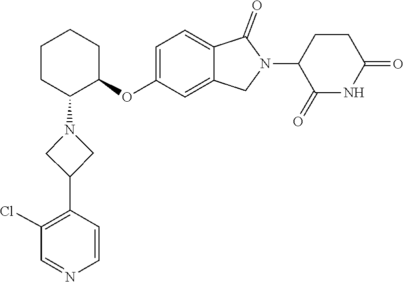 [SMILES: O=C1CCC(N2Cc3cc(O[C@@H]4CCCC[C@H]4N4CC(c5ccncc5Cl)C4)ccc3C2=O)C(=O)N1] 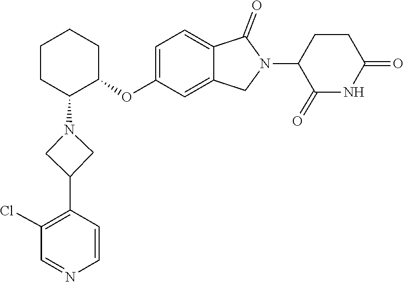 [SMILES: O=C1CCC(N2Cc3cc(O[C@H]4CCCC[C@H]4N4CC(c5ccncc5Cl)C4)ccc3C2=O)C(=O)N1] 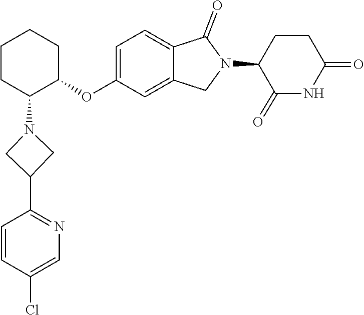 [SMILES: O=C1CC[C@H](N2Cc3cc(O[C@H]4CCCC[C@H]4N4CC(c5ccc(Cl)cn5)C4)ccc3C2=O)C(=O)N1] 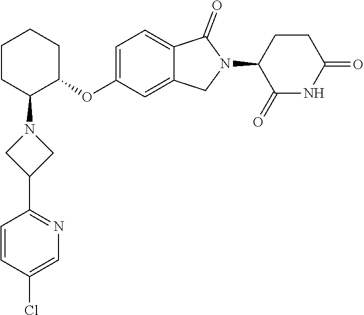 [SMILES: O=C1CC[C@H](N2Cc3cc(O[C@H]4CCCC[C@@H]4N4CC(c5ccc(Cl)cn5)C4)ccc3C2=O)C(=O)N1] 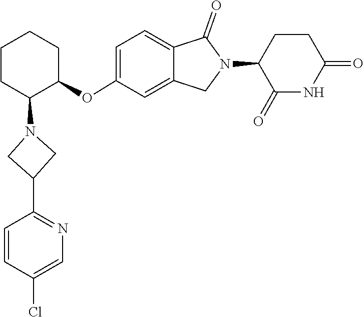 [SMILES: O=C1CC[C@H](N2Cc3cc(O[C@@H]4CCCC[C@@H]4N4CC(c5ccc(Cl)cn5)C4)ccc3C2=O)C(=O)N1] 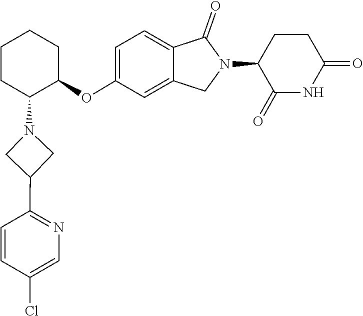 [SMILES: O=C1CC[C@H](N2Cc3cc(O[C@@H]4CCCC[C@H]4N4CC(c5ccc(Cl)cn5)C4)ccc3C2=O)C(=O)N1] 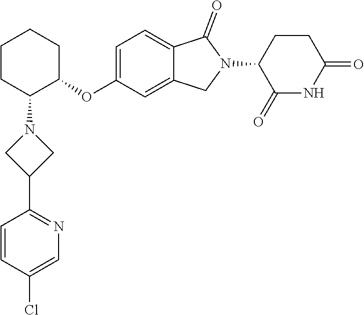 [SMILES: O=C1CC[C@@H](N2Cc3cc(O[C@H]4CCCC[C@H]4N4CC(c5ccc(Cl)cn5)C4)ccc3C2=O)C(=O)N1] 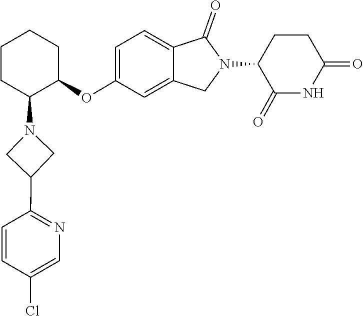 [SMILES: O=C1CC[C@@H](N2Cc3cc(O[C@@H]4CCCC[C@@H]4N4CC(c5ccc(Cl)cn5)C4)ccc3C2=O)C(=O)N1] 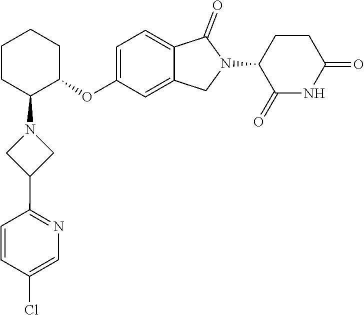 [SMILES: O=C1CC[C@@H](N2Cc3cc(O[C@H]4CCCC[C@@H]4N4CC(c5ccc(Cl)cn5)C4)ccc3C2=O)C(=O)N1] 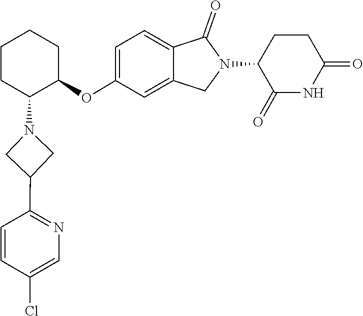 [SMILES: O=C1CC[C@@H](N2Cc3cc(O[C@@H]4CCCC[C@H]4N4CC(c5ccc(Cl)cn5)C4)ccc3C2=O)C(=O)N1] 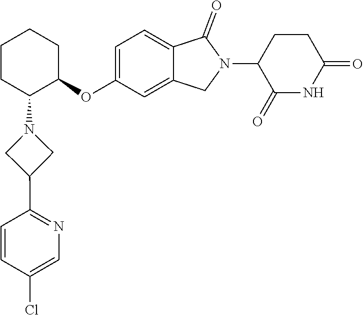 [SMILES: O=C1CCC(N2Cc3cc(O[C@@H]4CCCC[C@H]4N4CC(c5ccc(Cl)cn5)C4)ccc3C2=O)C(=O)N1] 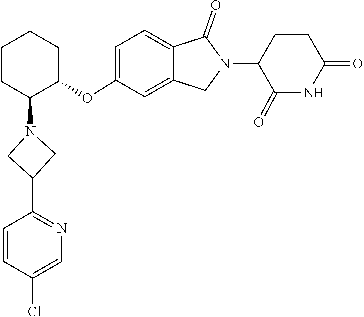 [SMILES: O=C1CCC(N2Cc3cc(O[C@H]4CCCC[C@@H]4N4CC(c5ccc(Cl)cn5)C4)ccc3C2=O)C(=O)N1] 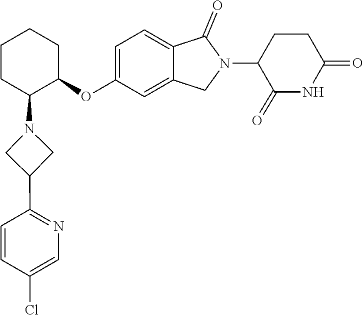 [SMILES: O=C1CCC(N2Cc3cc(O[C@@H]4CCCC[C@@H]4N4CC(c5ccc(Cl)cn5)C4)ccc3C2=O)C(=O)N1] 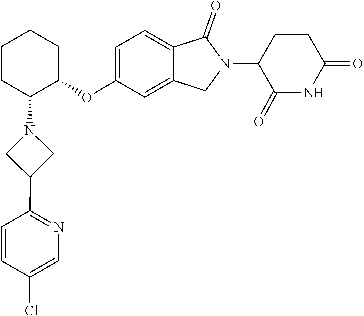 [SMILES: O=C1CCC(N2Cc3cc(O[C@H]4CCCC[C@H]4N4CC(c5ccc(Cl)cn5)C4)ccc3C2=O)C(=O)N1] 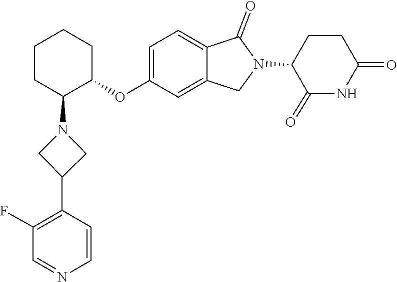 [SMILES: O=C1CC[C@@H](N2Cc3cc(O[C@H]4CCCC[C@@H]4N4CC(c5ccncc5F)C4)ccc3C2=O)C(=O)N1] 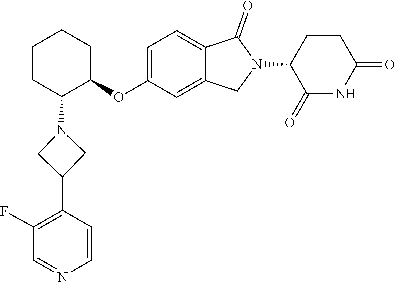 [SMILES: O=C1CC[C@@H](N2Cc3cc(O[C@@H]4CCCC[C@H]4N4CC(c5ccncc5F)C4)ccc3C2=O)C(=O)N1] 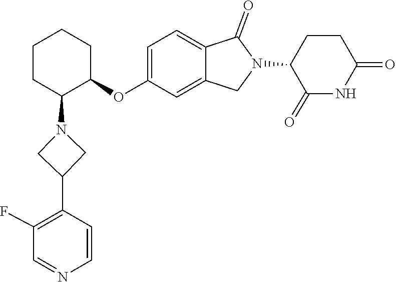 [SMILES: O=C1CC[C@@H](N2Cc3cc(O[C@@H]4CCCC[C@@H]4N4CC(c5ccncc5F)C4)ccc3C2=O)C(=O)N1] 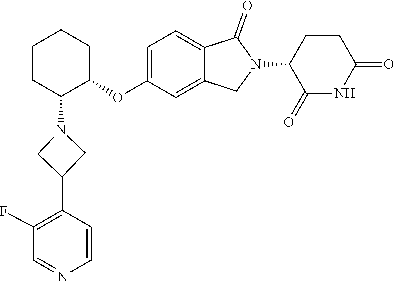 [SMILES: O=C1CC[C@@H](N2Cc3cc(O[C@H]4CCCC[C@H]4N4CC(c5ccncc5F)C4)ccc3C2=O)C(=O)N1] 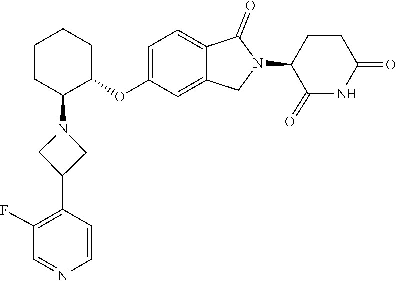 [SMILES: O=C1CC[C@H](N2Cc3cc(O[C@H]4CCCC[C@@H]4N4CC(c5ccncc5F)C4)ccc3C2=O)C(=O)N1] 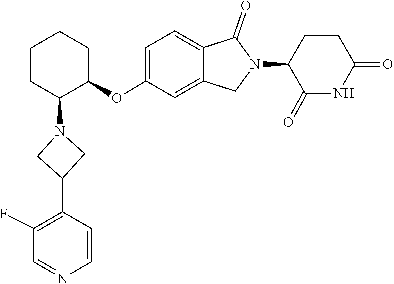 [SMILES: O=C1CC[C@H](N2Cc3cc(O[C@@H]4CCCC[C@@H]4N4CC(c5ccncc5F)C4)ccc3C2=O)C(=O)N1] 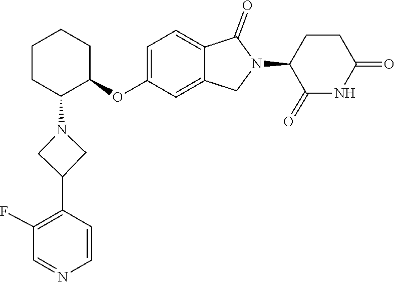 [SMILES: O=C1CC[C@H](N2Cc3cc(O[C@@H]4CCCC[C@H]4N4CC(c5ccncc5F)C4)ccc3C2=O)C(=O)N1] 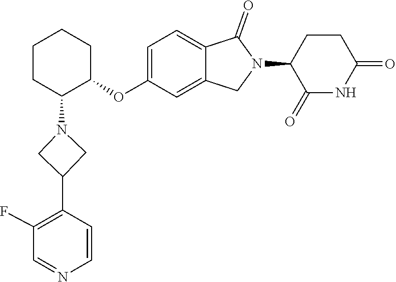 [SMILES: O=C1CC[C@H](N2Cc3cc(O[C@H]4CCCC[C@H]4N4CC(c5ccncc5F)C4)ccc3C2=O)C(=O)N1] 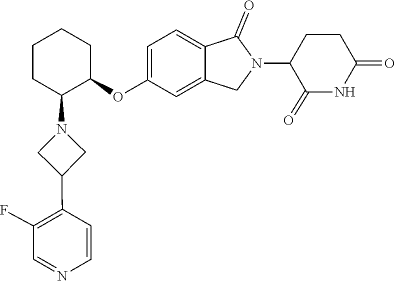 [SMILES: O=C1CCC(N2Cc3cc(O[C@@H]4CCCC[C@@H]4N4CC(c5ccncc5F)C4)ccc3C2=O)C(=O)N1] 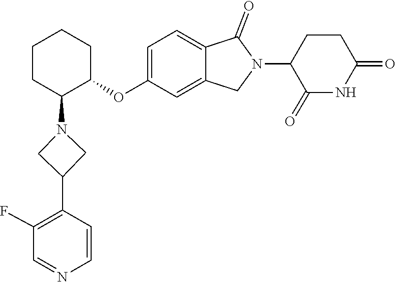 [SMILES: O=C1CCC(N2Cc3cc(O[C@H]4CCCC[C@@H]4N4CC(c5ccncc5F)C4)ccc3C2=O)C(=O)N1] 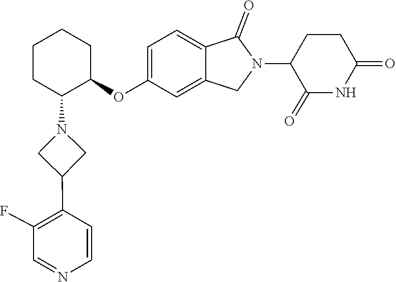 [SMILES: O=C1CCC(N2Cc3cc(O[C@@H]4CCCC[C@H]4N4CC(c5ccncc5F)C4)ccc3C2=O)C(=O)N1] 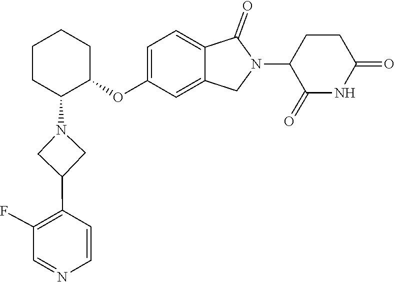 [SMILES: O=C1CCC(N2Cc3cc(O[C@H]4CCCC[C@H]4N4CC(c5ccncc5F)C4)ccc3C2=O)C(=O)N1] 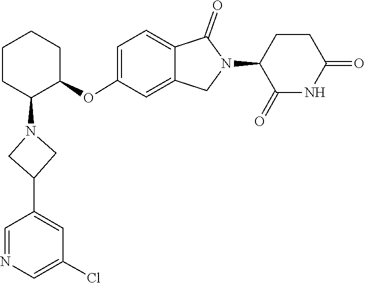 [SMILES: O=C1CC[C@H](N2Cc3cc(O[C@@H]4CCCC[C@@H]4N4CC(c5cncc(Cl)c5)C4)ccc3C2=O)C(=O)N1] 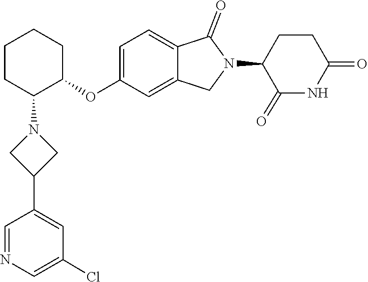 [SMILES: O=C1CC[C@H](N2Cc3cc(O[C@H]4CCCC[C@H]4N4CC(c5cncc(Cl)c5)C4)ccc3C2=O)C(=O)N1] 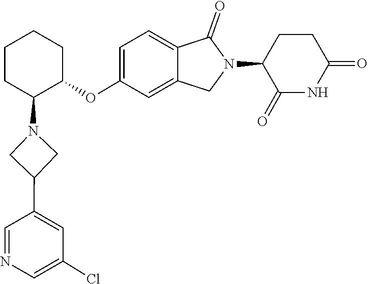 [SMILES: O=C1CC[C@H](N2Cc3cc(O[C@H]4CCCC[C@@H]4N4CC(c5cncc(Cl)c5)C4)ccc3C2=O)C(=O)N1] 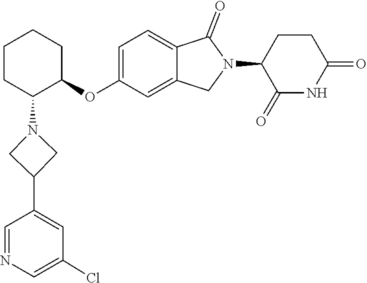 [SMILES: O=C1CC[C@H](N2Cc3cc(O[C@@H]4CCCC[C@H]4N4CC(c5cncc(Cl)c5)C4)ccc3C2=O)C(=O)N1] 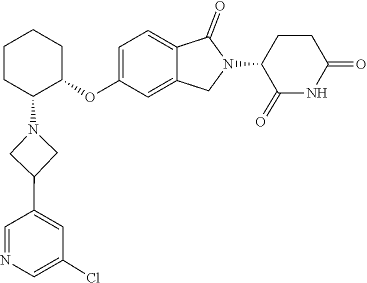 [SMILES: O=C1CC[C@@H](N2Cc3cc(O[C@H]4CCCC[C@H]4N4CC(c5cncc(Cl)c5)C4)ccc3C2=O)C(=O)N1] 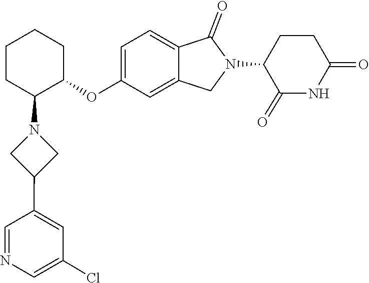 [SMILES: O=C1CC[C@@H](N2Cc3cc(O[C@H]4CCCC[C@@H]4N4CC(c5cncc(Cl)c5)C4)ccc3C2=O)C(=O)N1] 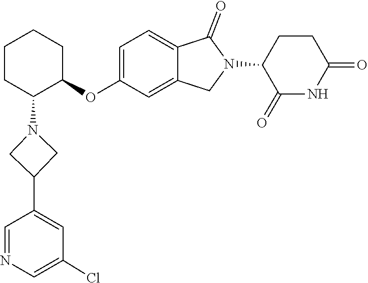 [SMILES: O=C1CC[C@@H](N2Cc3cc(O[C@@H]4CCCC[C@H]4N4CC(c5cncc(Cl)c5)C4)ccc3C2=O)C(=O)N1] 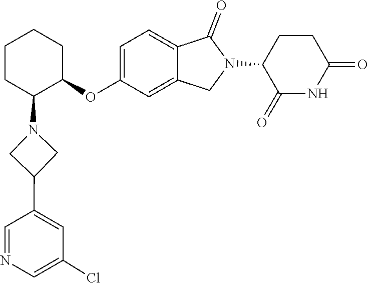 [SMILES: O=C1CC[C@@H](N2Cc3cc(O[C@@H]4CCCC[C@@H]4N4CC(c5cncc(Cl)c5)C4)ccc3C2=O)C(=O)N1] 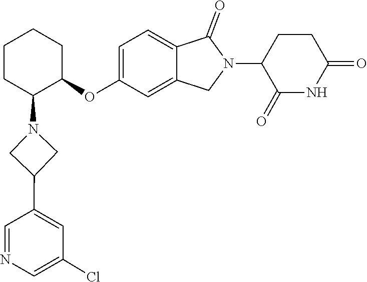 [SMILES: O=C1CCC(N2Cc3cc(O[C@@H]4CCCC[C@@H]4N4CC(c5cncc(Cl)c5)C4)ccc3C2=O)C(=O)N1] 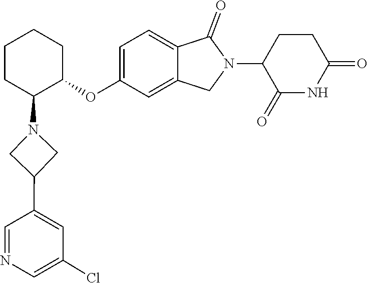 [SMILES: O=C1CCC(N2Cc3cc(O[C@H]4CCCC[C@@H]4N4CC(c5cncc(Cl)c5)C4)ccc3C2=O)C(=O)N1] 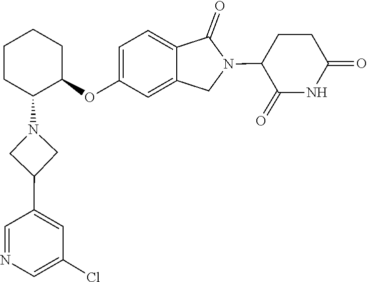 [SMILES: O=C1CCC(N2Cc3cc(O[C@@H]4CCCC[C@H]4N4CC(c5cncc(Cl)c5)C4)ccc3C2=O)C(=O)N1] 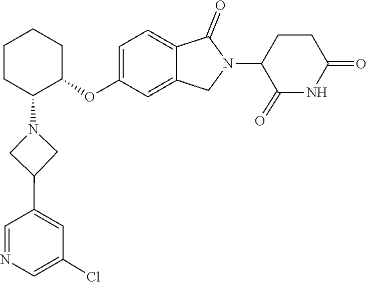 [SMILES: O=C1CCC(N2Cc3cc(O[C@H]4CCCC[C@H]4N4CC(c5cncc(Cl)c5)C4)ccc3C2=O)C(=O)N1] 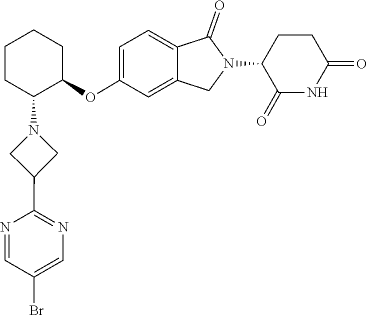 [SMILES: O=C1CC[C@@H](N2Cc3cc(O[C@@H]4CCCC[C@H]4N4CC(c5ncc(Br)cn5)C4)ccc3C2=O)C(=O)N1] 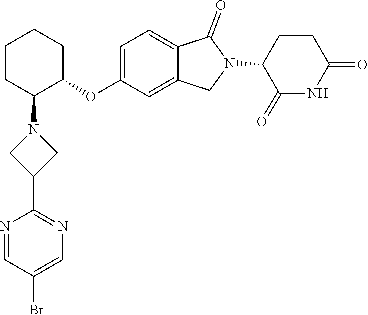 [SMILES: O=C1CC[C@@H](N2Cc3cc(O[C@H]4CCCC[C@@H]4N4CC(c5ncc(Br)cn5)C4)ccc3C2=O)C(=O)N1] 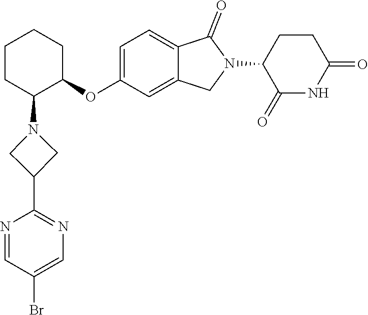 [SMILES: O=C1CC[C@@H](N2Cc3cc(O[C@@H]4CCCC[C@@H]4N4CC(c5ncc(Br)cn5)C4)ccc3C2=O)C(=O)N1] 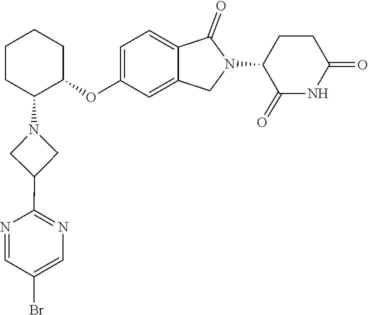 [SMILES: O=C1CC[C@@H](N2Cc3cc(O[C@H]4CCCC[C@H]4N4CC(c5ncc(Br)cn5)C4)ccc3C2=O)C(=O)N1] 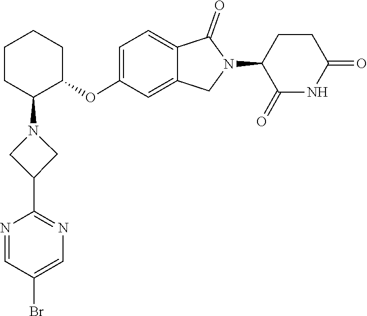 [SMILES: O=C1CC[C@H](N2Cc3cc(O[C@H]4CCCC[C@@H]4N4CC(c5ncc(Br)cn5)C4)ccc3C2=O)C(=O)N1] 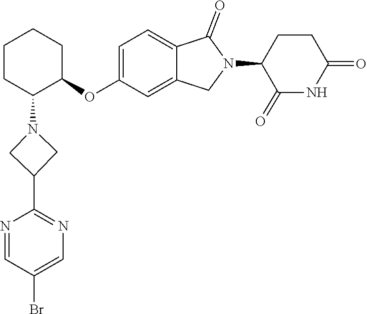 [SMILES: O=C1CC[C@H](N2Cc3cc(O[C@@H]4CCCC[C@H]4N4CC(c5ncc(Br)cn5)C4)ccc3C2=O)C(=O)N1] 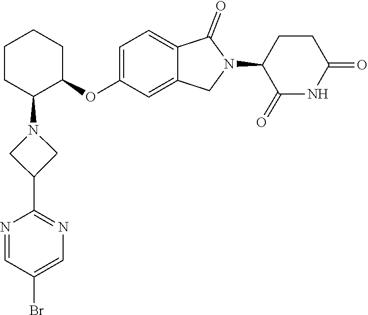 [SMILES: O=C1CC[C@H](N2Cc3cc(O[C@@H]4CCCC[C@@H]4N4CC(c5ncc(Br)cn5)C4)ccc3C2=O)C(=O)N1] 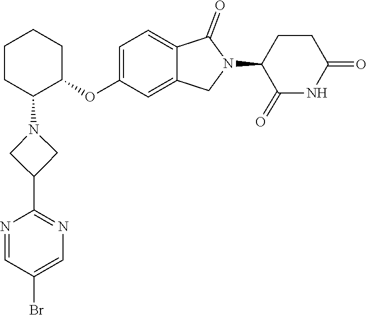 [SMILES: O=C1CC[C@H](N2Cc3cc(O[C@H]4CCCC[C@H]4N4CC(c5ncc(Br)cn5)C4)ccc3C2=O)C(=O)N1] 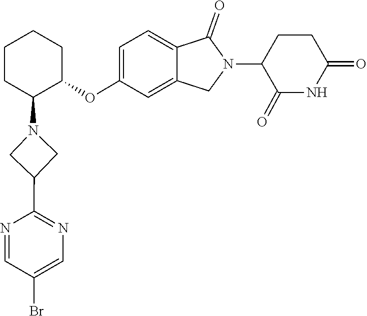 [SMILES: O=C1CCC(N2Cc3cc(O[C@H]4CCCC[C@@H]4N4CC(c5ncc(Br)cn5)C4)ccc3C2=O)C(=O)N1] 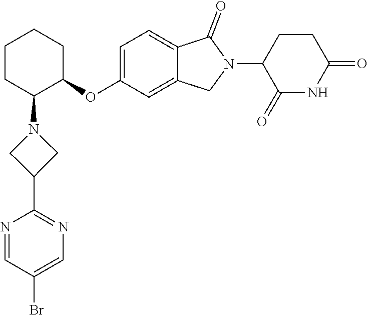 [SMILES: O=C1CCC(N2Cc3cc(O[C@@H]4CCCC[C@@H]4N4CC(c5ncc(Br)cn5)C4)ccc3C2=O)C(=O)N1] 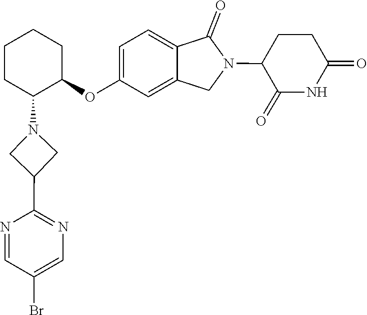 [SMILES: O=C1CCC(N2Cc3cc(O[C@@H]4CCCC[C@H]4N4CC(c5ncc(Br)cn5)C4)ccc3C2=O)C(=O)N1] 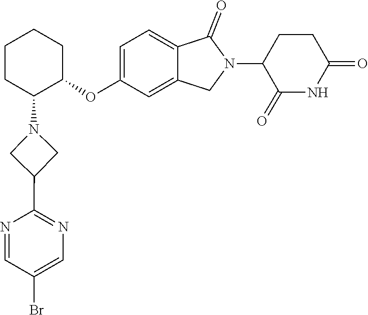 [SMILES: O=C1CCC(N2Cc3cc(O[C@H]4CCCC[C@H]4N4CC(c5ncc(Br)cn5)C4)ccc3C2=O)C(=O)N1] 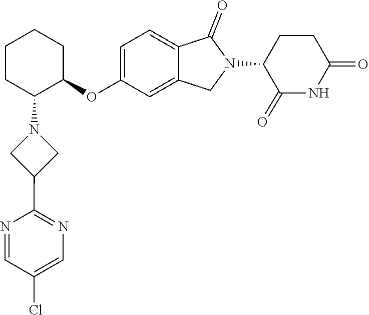 [SMILES: O=C1CC[C@@H](N2Cc3cc(O[C@@H]4CCCC[C@H]4N4CC(c5ncc(Cl)cn5)C4)ccc3C2=O)C(=O)N1] 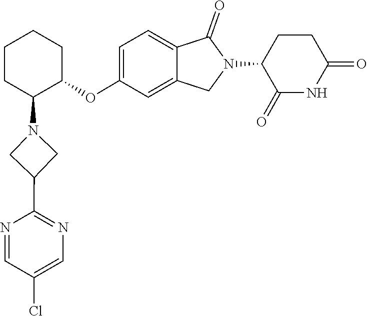 [SMILES: O=C1CC[C@@H](N2Cc3cc(O[C@H]4CCCC[C@@H]4N4CC(c5ncc(Cl)cn5)C4)ccc3C2=O)C(=O)N1] 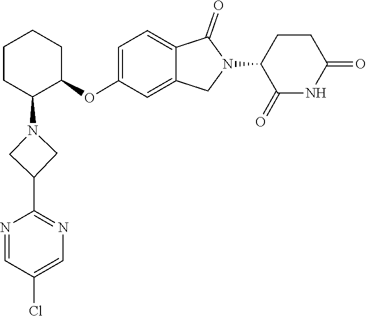 [SMILES: O=C1CC[C@@H](N2Cc3cc(O[C@@H]4CCCC[C@@H]4N4CC(c5ncc(Cl)cn5)C4)ccc3C2=O)C(=O)N1] 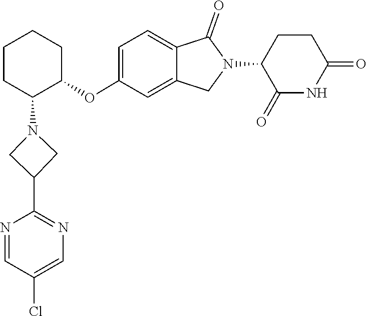 [SMILES: O=C1CC[C@@H](N2Cc3cc(O[C@H]4CCCC[C@H]4N4CC(c5ncc(Cl)cn5)C4)ccc3C2=O)C(=O)N1] 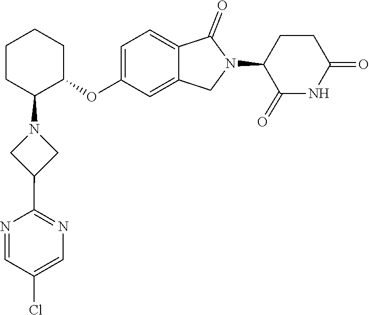 [SMILES: O=C1CC[C@H](N2Cc3cc(O[C@H]4CCCC[C@@H]4N4CC(c5ncc(Cl)cn5)C4)ccc3C2=O)C(=O)N1] 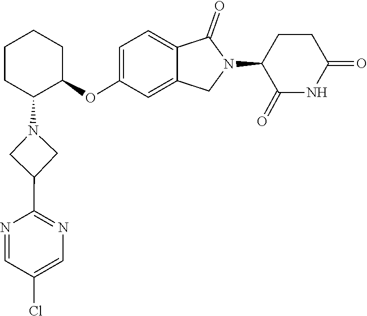 [SMILES: O=C1CC[C@H](N2Cc3cc(O[C@@H]4CCCC[C@H]4N4CC(c5ncc(Cl)cn5)C4)ccc3C2=O)C(=O)N1] 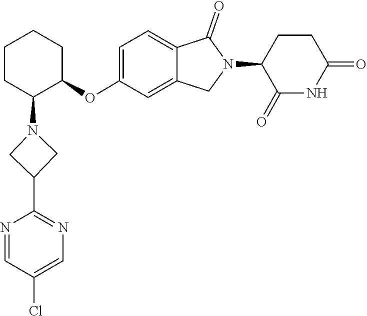 [SMILES: O=C1CC[C@H](N2Cc3cc(O[C@@H]4CCCC[C@@H]4N4CC(c5ncc(Cl)cn5)C4)ccc3C2=O)C(=O)N1] 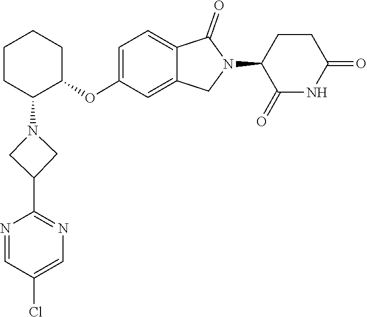 [SMILES: O=C1CC[C@H](N2Cc3cc(O[C@H]4CCCC[C@H]4N4CC(c5ncc(Cl)cn5)C4)ccc3C2=O)C(=O)N1] 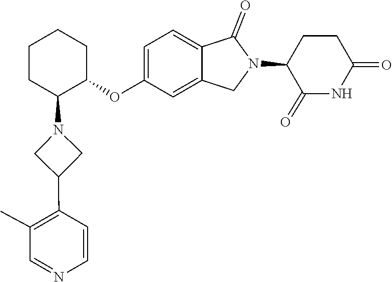 [SMILES: Cc1cnccc1C1CN([C@H]2CCCC[C@@H]2Oc2ccc3c(c2)CN([C@H]2CCC(=O)NC2=O)C3=O)C1] 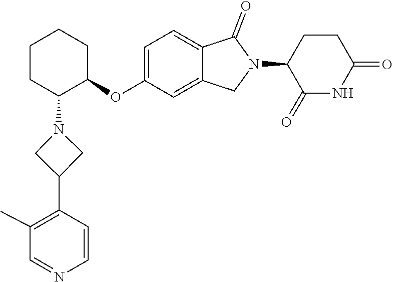 [SMILES: Cc1cnccc1C1CN([C@@H]2CCCC[C@H]2Oc2ccc3c(c2)CN([C@H]2CCC(=O)NC2=O)C3=O)C1] 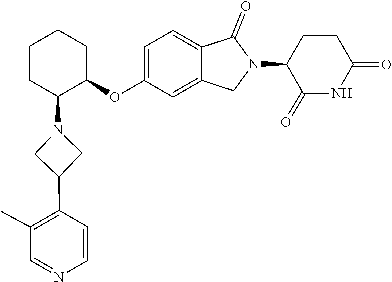 [SMILES: Cc1cnccc1C1CN([C@H]2CCCC[C@H]2Oc2ccc3c(c2)CN([C@H]2CCC(=O)NC2=O)C3=O)C1] 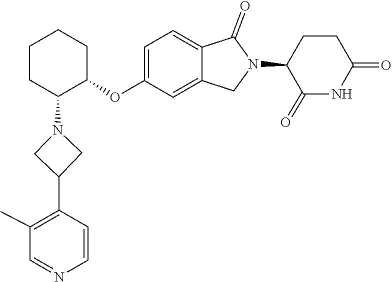 [SMILES: Cc1cnccc1C1CN([C@@H]2CCCC[C@@H]2Oc2ccc3c(c2)CN([C@H]2CCC(=O)NC2=O)C3=O)C1] 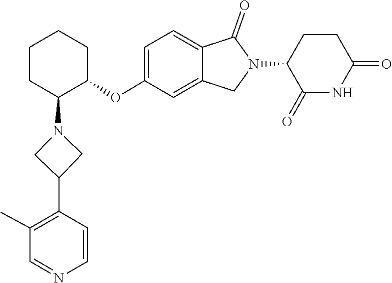 [SMILES: Cc1cnccc1C1CN([C@H]2CCCC[C@@H]2Oc2ccc3c(c2)CN([C@@H]2CCC(=O)NC2=O)C3=O)C1] 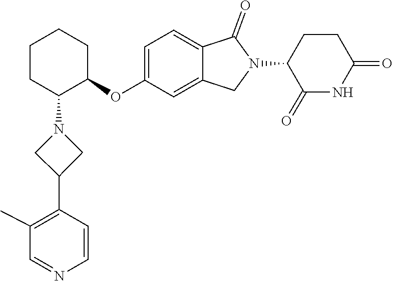 [SMILES: Cc1cnccc1C1CN([C@@H]2CCCC[C@H]2Oc2ccc3c(c2)CN([C@@H]2CCC(=O)NC2=O)C3=O)C1] 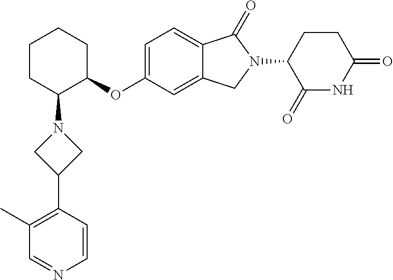 [SMILES: Cc1cnccc1C1CN([C@H]2CCCC[C@H]2Oc2ccc3c(c2)CN([C@@H]2CCC(=O)NC2=O)C3=O)C1] 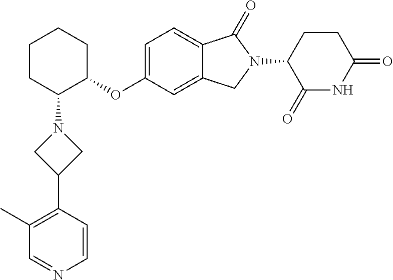 [SMILES: Cc1cnccc1C1CN([C@@H]2CCCC[C@@H]2Oc2ccc3c(c2)CN([C@@H]2CCC(=O)NC2=O)C3=O)C1] 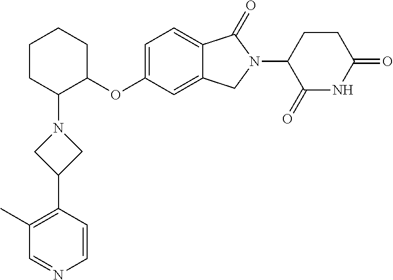 [SMILES: Cc1cnccc1C1CN(C2CCCCC2Oc2ccc3c(c2)CN(C2CCC(=O)NC2=O)C3=O)C1] 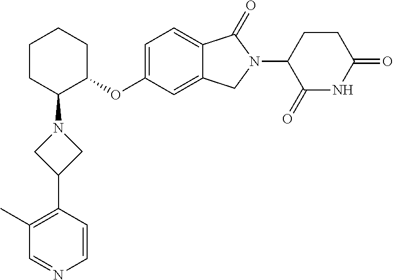 [SMILES: Cc1cnccc1C1CN([C@H]2CCCC[C@@H]2Oc2ccc3c(c2)CN(C2CCC(=O)NC2=O)C3=O)C1] 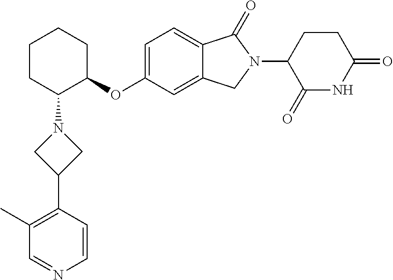 [SMILES: Cc1cnccc1C1CN([C@@H]2CCCC[C@H]2Oc2ccc3c(c2)CN(C2CCC(=O)NC2=O)C3=O)C1] 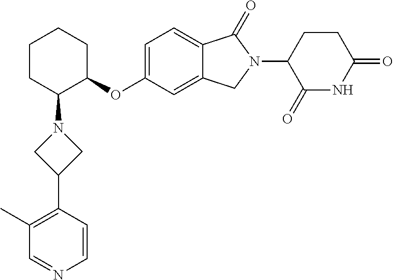 [SMILES: Cc1cnccc1C1CN([C@H]2CCCC[C@H]2Oc2ccc3c(c2)CN(C2CCC(=O)NC2=O)C3=O)C1] 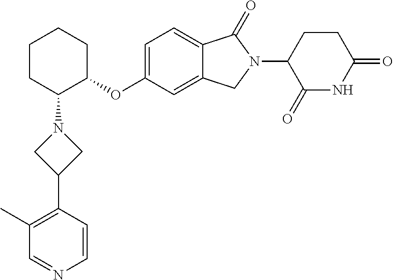 [SMILES: Cc1cnccc1C1CN([C@@H]2CCCC[C@@H]2Oc2ccc3c(c2)CN(C2CCC(=O)NC2=O)C3=O)C1] 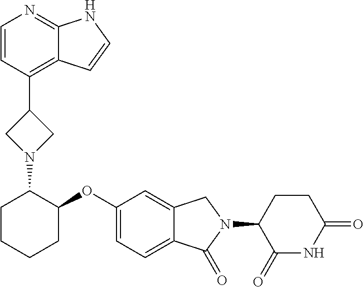 [SMILES: O=C1CC[C@H](N2Cc3cc(O[C@H]4CCCC[C@@H]4N4CC(c5ccnc6[nH]ccc56)C4)ccc3C2=O)C(=O)N1] 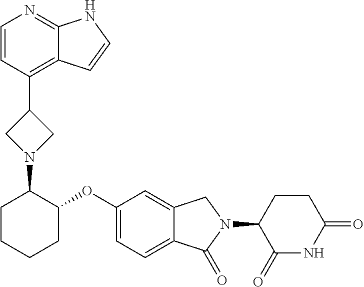 [SMILES: O=C1CC[C@H](N2Cc3cc(O[C@@H]4CCCC[C@H]4N4CC(c5ccnc6[nH]ccc56)C4)ccc3C2=O)C(=O)N1] 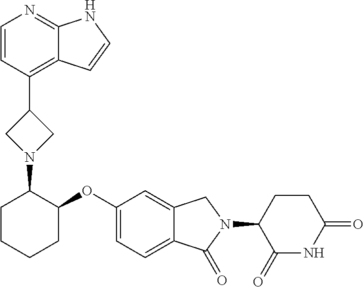 [SMILES: O=C1CC[C@H](N2Cc3cc(O[C@H]4CCCC[C@H]4N4CC(c5ccnc6[nH]ccc56)C4)ccc3C2=O)C(=O)N1] 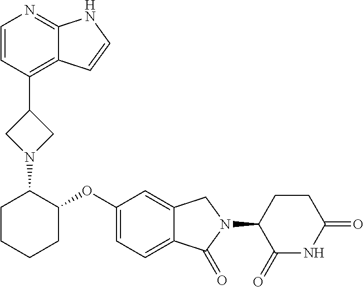 [SMILES: O=C1CC[C@H](N2Cc3cc(O[C@@H]4CCCC[C@@H]4N4CC(c5ccnc6[nH]ccc56)C4)ccc3C2=O)C(=O)N1] 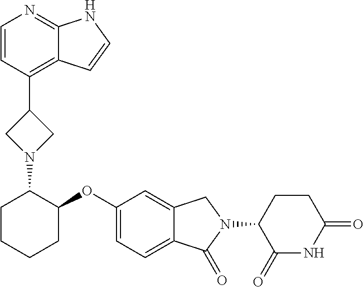 [SMILES: O=C1CC[C@@H](N2Cc3cc(O[C@H]4CCCC[C@@H]4N4CC(c5ccnc6[nH]ccc56)C4)ccc3C2=O)C(=O)N1] 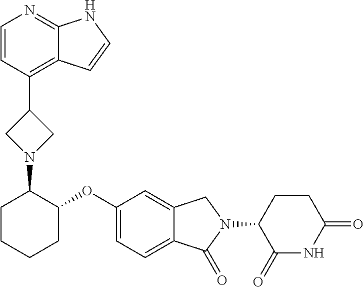 [SMILES: O=C1CC[C@@H](N2Cc3cc(O[C@@H]4CCCC[C@H]4N4CC(c5ccnc6[nH]ccc56)C4)ccc3C2=O)C(=O)N1] 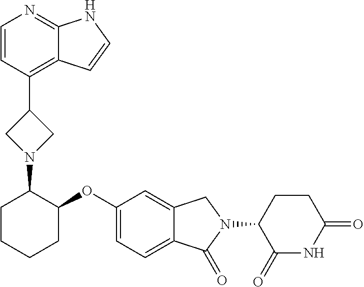 [SMILES: O=C1CC[C@@H](N2Cc3cc(O[C@H]4CCCC[C@H]4N4CC(c5ccnc6[nH]ccc56)C4)ccc3C2=O)C(=O)N1] 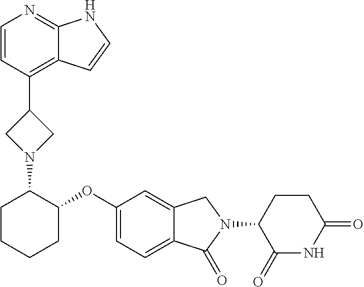 [SMILES: O=C1CC[C@@H](N2Cc3cc(O[C@@H]4CCCC[C@@H]4N4CC(c5ccnc6[nH]ccc56)C4)ccc3C2=O)C(=O)N1] 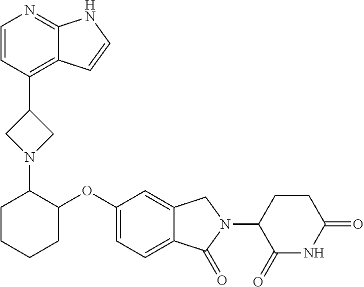 [SMILES: O=C1CCC(N2Cc3cc(OC4CCCCC4N4CC(c5ccnc6[nH]ccc56)C4)ccc3C2=O)C(=O)N1] 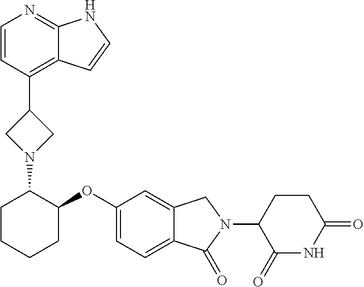 [SMILES: O=C1CCC(N2Cc3cc(O[C@H]4CCCC[C@@H]4N4CC(c5ccnc6[nH]ccc56)C4)ccc3C2=O)C(=O)N1] 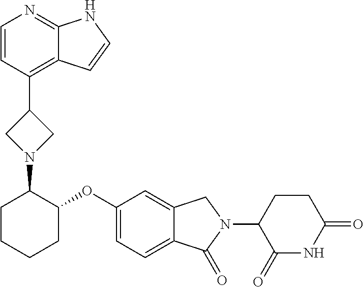 [SMILES: O=C1CCC(N2Cc3cc(O[C@@H]4CCCC[C@H]4N4CC(c5ccnc6[nH]ccc56)C4)ccc3C2=O)C(=O)N1] 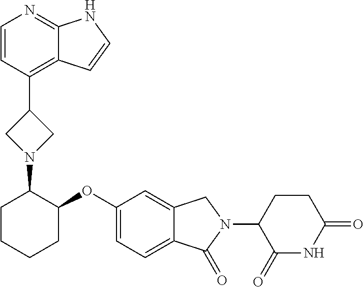 [SMILES: O=C1CCC(N2Cc3cc(O[C@H]4CCCC[C@H]4N4CC(c5ccnc6[nH]ccc56)C4)ccc3C2=O)C(=O)N1] 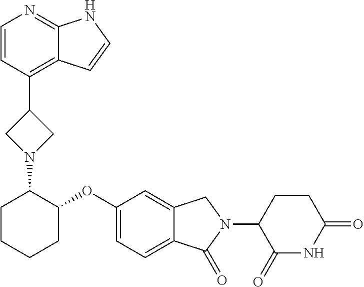 [SMILES: O=C1CCC(N2Cc3cc(O[C@@H]4CCCC[C@@H]4N4CC(c5ccnc6[nH]ccc56)C4)ccc3C2=O)C(=O)N1] 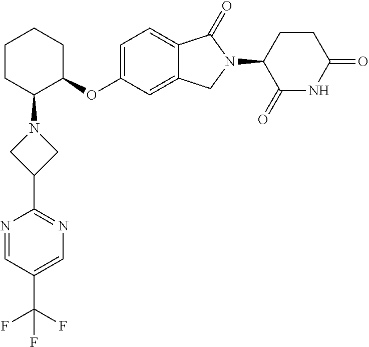 [SMILES: O=C1CC[C@H](N2Cc3cc(O[C@@H]4CCCC[C@@H]4N4CC(c5ncc(C(F)(F)F)cn5)C4)ccc3C2=O)C(=O)N1] 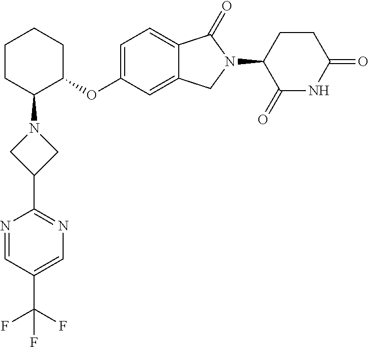 [SMILES: O=C1CC[C@H](N2Cc3cc(O[C@H]4CCCC[C@@H]4N4CC(c5ncc(C(F)(F)F)cn5)C4)ccc3C2=O)C(=O)N1] 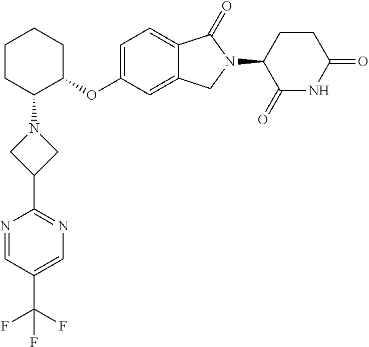 [SMILES: O=C1CC[C@H](N2Cc3cc(O[C@H]4CCCC[C@H]4N4CC(c5ncc(C(F)(F)F)cn5)C4)ccc3C2=O)C(=O)N1] 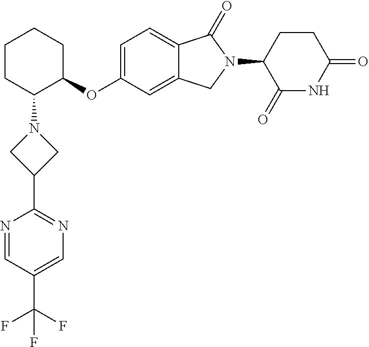 [SMILES: O=C1CC[C@H](N2Cc3cc(O[C@@H]4CCCC[C@H]4N4CC(c5ncc(C(F)(F)F)cn5)C4)ccc3C2=O)C(=O)N1] 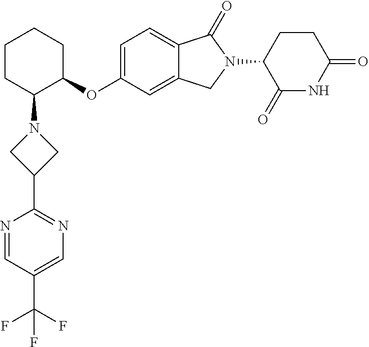 [SMILES: O=C1CC[C@@H](N2Cc3cc(O[C@@H]4CCCC[C@@H]4N4CC(c5ncc(C(F)(F)F)cn5)C4)ccc3C2=O)C(=O)N1] 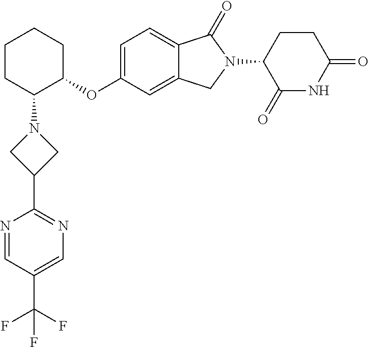 [SMILES: O=C1CC[C@@H](N2Cc3cc(O[C@H]4CCCC[C@H]4N4CC(c5ncc(C(F)(F)F)cn5)C4)ccc3C2=O)C(=O)N1] 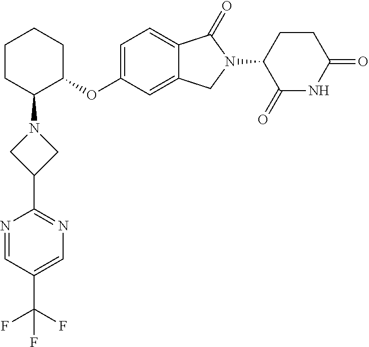 [SMILES: O=C1CC[C@@H](N2Cc3cc(O[C@H]4CCCC[C@@H]4N4CC(c5ncc(C(F)(F)F)cn5)C4)ccc3C2=O)C(=O)N1] 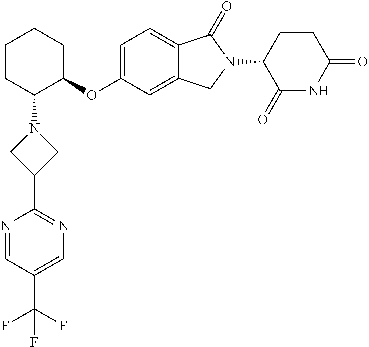 [SMILES: O=C1CC[C@@H](N2Cc3cc(O[C@@H]4CCCC[C@H]4N4CC(c5ncc(C(F)(F)F)cn5)C4)ccc3C2=O)C(=O)N1] 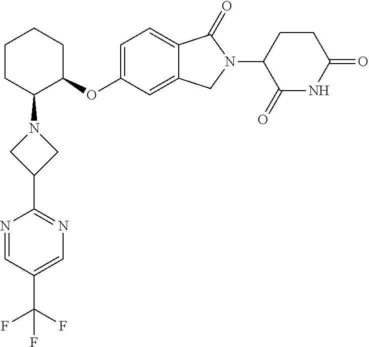 [SMILES: O=C1CCC(N2Cc3cc(O[C@@H]4CCCC[C@@H]4N4CC(c5ncc(C(F)(F)F)cn5)C4)ccc3C2=O)C(=O)N1] 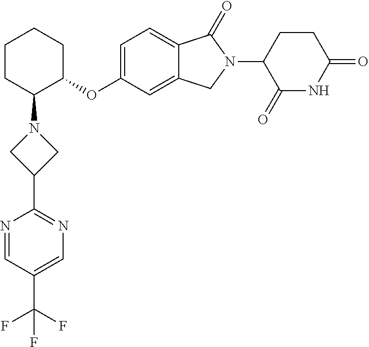 [SMILES: O=C1CCC(N2Cc3cc(O[C@H]4CCCC[C@@H]4N4CC(c5ncc(C(F)(F)F)cn5)C4)ccc3C2=O)C(=O)N1] 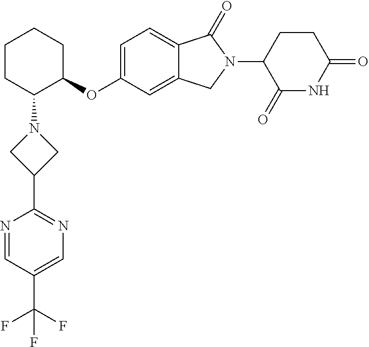 [SMILES: O=C1CCC(N2Cc3cc(O[C@@H]4CCCC[C@H]4N4CC(c5ncc(C(F)(F)F)cn5)C4)ccc3C2=O)C(=O)N1] 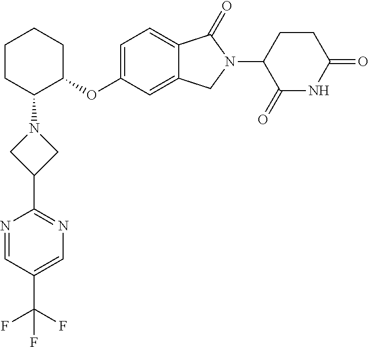 [SMILES: O=C1CCC(N2Cc3cc(O[C@H]4CCCC[C@H]4N4CC(c5ncc(C(F)(F)F)cn5)C4)ccc3C2=O)C(=O)N1] 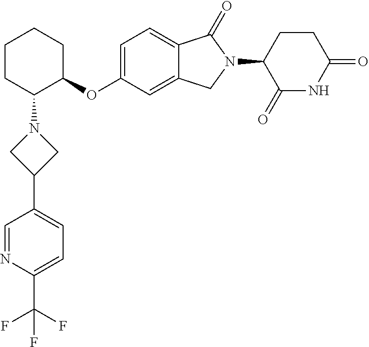 [SMILES: O=C1CC[C@H](N2Cc3cc(O[C@@H]4CCCC[C@H]4N4CC(c5ccc(C(F)(F)F)nc5)C4)ccc3C2=O)C(=O)N1] 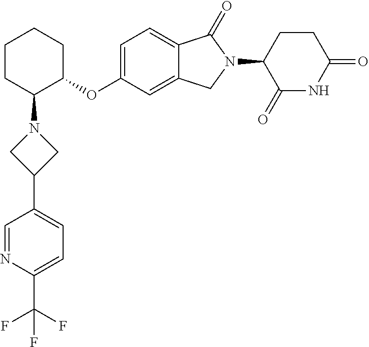 [SMILES: O=C1CC[C@H](N2Cc3cc(O[C@H]4CCCC[C@@H]4N4CC(c5ccc(C(F)(F)F)nc5)C4)ccc3C2=O)C(=O)N1] 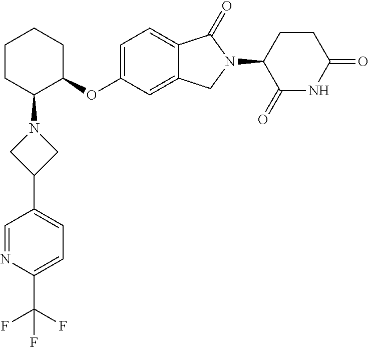 [SMILES: O=C1CC[C@H](N2Cc3cc(O[C@@H]4CCCC[C@@H]4N4CC(c5ccc(C(F)(F)F)nc5)C4)ccc3C2=O)C(=O)N1] 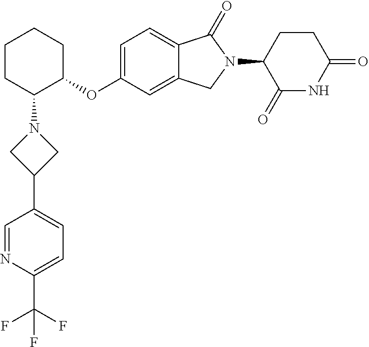 [SMILES: O=C1CC[C@H](N2Cc3cc(O[C@H]4CCCC[C@H]4N4CC(c5ccc(C(F)(F)F)nc5)C4)ccc3C2=O)C(=O)N1] 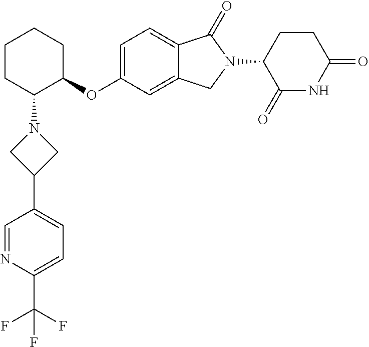 [SMILES: O=C1CC[C@@H](N2Cc3cc(O[C@@H]4CCCC[C@H]4N4CC(c5ccc(C(F)(F)F)nc5)C4)ccc3C2=O)C(=O)N1] 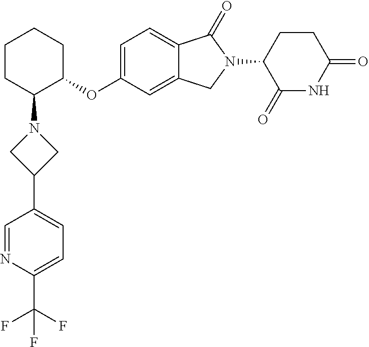 [SMILES: O=C1CC[C@@H](N2Cc3cc(O[C@H]4CCCC[C@@H]4N4CC(c5ccc(C(F)(F)F)nc5)C4)ccc3C2=O)C(=O)N1] 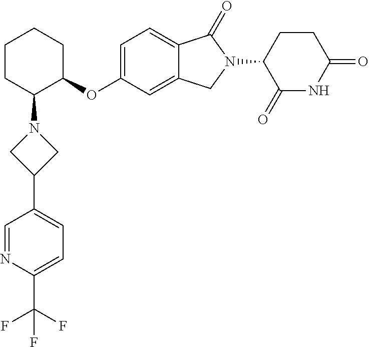 [SMILES: O=C1CC[C@@H](N2Cc3cc(O[C@@H]4CCCC[C@@H]4N4CC(c5ccc(C(F)(F)F)nc5)C4)ccc3C2=O)C(=O)N1] 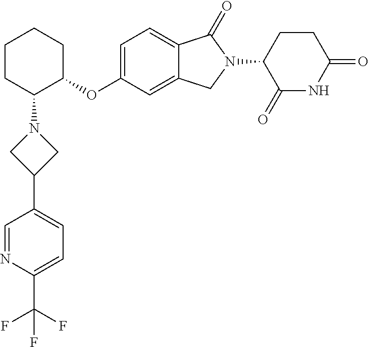 [SMILES: O=C1CC[C@@H](N2Cc3cc(O[C@H]4CCCC[C@H]4N4CC(c5ccc(C(F)(F)F)nc5)C4)ccc3C2=O)C(=O)N1] 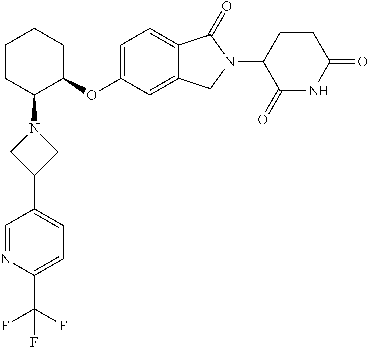 [SMILES: O=C1CCC(N2Cc3cc(O[C@@H]4CCCC[C@@H]4N4CC(c5ccc(C(F)(F)F)nc5)C4)ccc3C2=O)C(=O)N1] 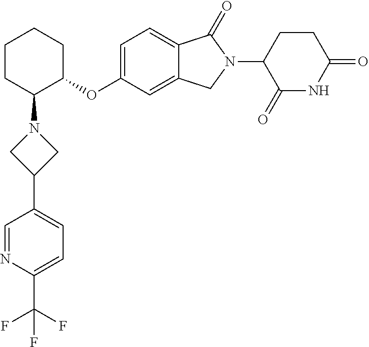 [SMILES: O=C1CCC(N2Cc3cc(O[C@H]4CCCC[C@@H]4N4CC(c5ccc(C(F)(F)F)nc5)C4)ccc3C2=O)C(=O)N1] 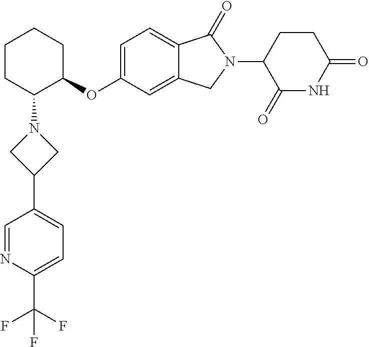 [SMILES: O=C1CCC(N2Cc3cc(O[C@@H]4CCCC[C@H]4N4CC(c5ccc(C(F)(F)F)nc5)C4)ccc3C2=O)C(=O)N1] 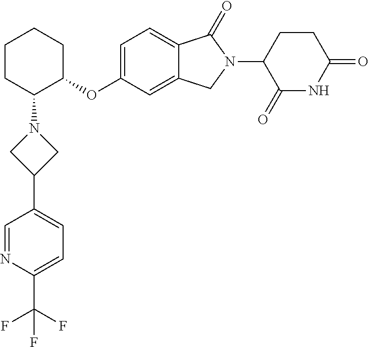 [SMILES: O=C1CCC(N2Cc3cc(O[C@H]4CCCC[C@H]4N4CC(c5ccc(C(F)(F)F)nc5)C4)ccc3C2=O)C(=O)N1] 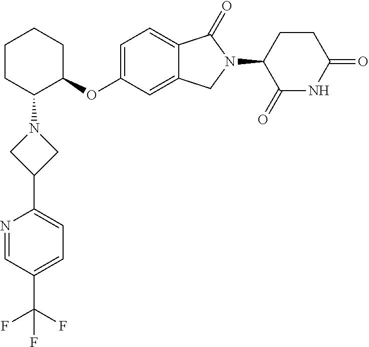 [SMILES: O=C1CC[C@H](N2Cc3cc(O[C@@H]4CCCC[C@H]4N4CC(c5ccc(C(F)(F)F)cn5)C4)ccc3C2=O)C(=O)N1] 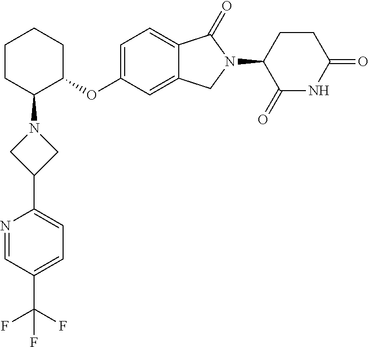 [SMILES: O=C1CC[C@H](N2Cc3cc(O[C@H]4CCCC[C@@H]4N4CC(c5ccc(C(F)(F)F)cn5)C4)ccc3C2=O)C(=O)N1] 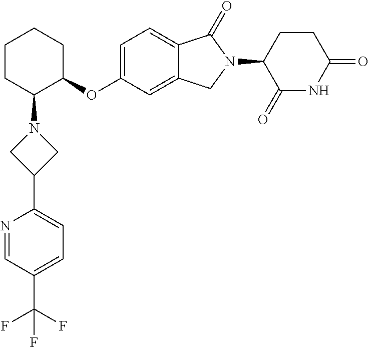 [SMILES: O=C1CC[C@H](N2Cc3cc(O[C@@H]4CCCC[C@@H]4N4CC(c5ccc(C(F)(F)F)cn5)C4)ccc3C2=O)C(=O)N1] 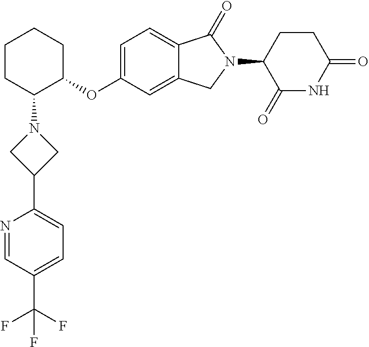 [SMILES: O=C1CC[C@H](N2Cc3cc(O[C@H]4CCCC[C@H]4N4CC(c5ccc(C(F)(F)F)cn5)C4)ccc3C2=O)C(=O)N1] 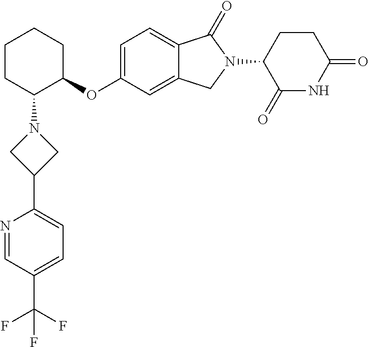 [SMILES: O=C1CC[C@@H](N2Cc3cc(O[C@@H]4CCCC[C@H]4N4CC(c5ccc(C(F)(F)F)cn5)C4)ccc3C2=O)C(=O)N1] 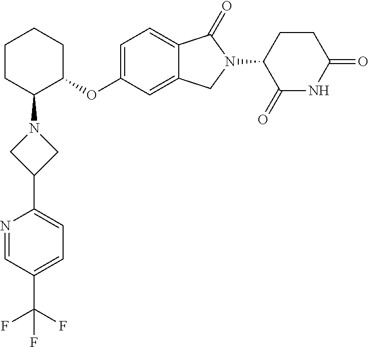 [SMILES: O=C1CC[C@@H](N2Cc3cc(O[C@H]4CCCC[C@@H]4N4CC(c5ccc(C(F)(F)F)cn5)C4)ccc3C2=O)C(=O)N1] 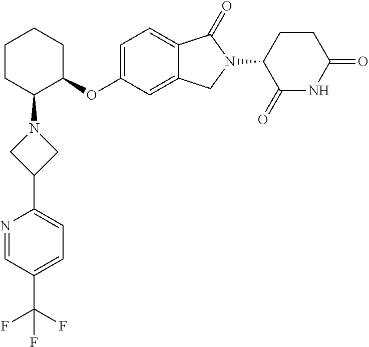 [SMILES: O=C1CC[C@@H](N2Cc3cc(O[C@@H]4CCCC[C@@H]4N4CC(c5ccc(C(F)(F)F)cn5)C4)ccc3C2=O)C(=O)N1] 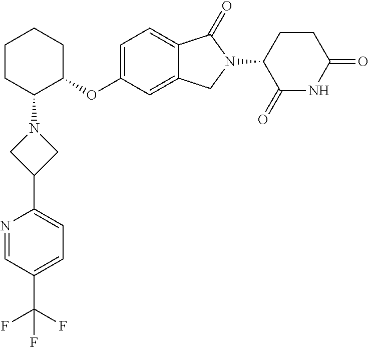 [SMILES: O=C1CC[C@@H](N2Cc3cc(O[C@H]4CCCC[C@H]4N4CC(c5ccc(C(F)(F)F)cn5)C4)ccc3C2=O)C(=O)N1] 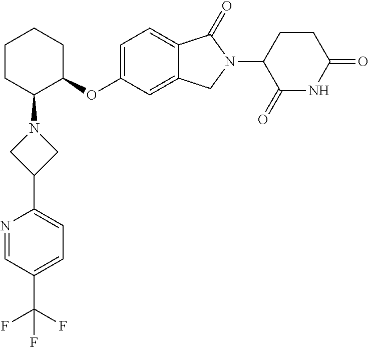 [SMILES: O=C1CCC(N2Cc3cc(O[C@@H]4CCCC[C@@H]4N4CC(c5ccc(C(F)(F)F)cn5)C4)ccc3C2=O)C(=O)N1] 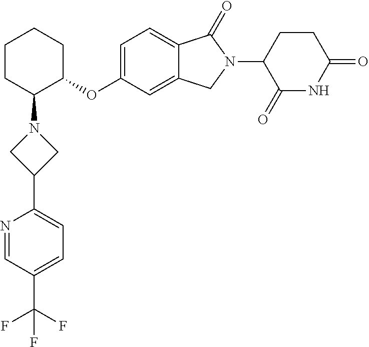 [SMILES: O=C1CCC(N2Cc3cc(O[C@H]4CCCC[C@@H]4N4CC(c5ccc(C(F)(F)F)cn5)C4)ccc3C2=O)C(=O)N1] 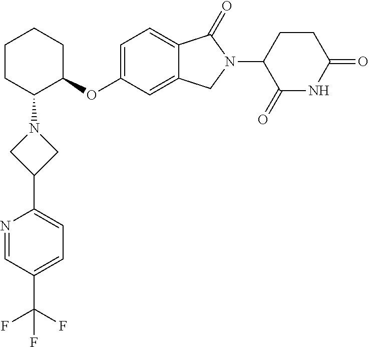 [SMILES: O=C1CCC(N2Cc3cc(O[C@@H]4CCCC[C@H]4N4CC(c5ccc(C(F)(F)F)cn5)C4)ccc3C2=O)C(=O)N1] 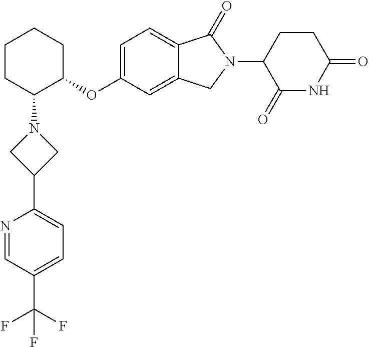 [SMILES: O=C1CCC(N2Cc3cc(O[C@H]4CCCC[C@H]4N4CC(c5ccc(C(F)(F)F)cn5)C4)ccc3C2=O)C(=O)N1] 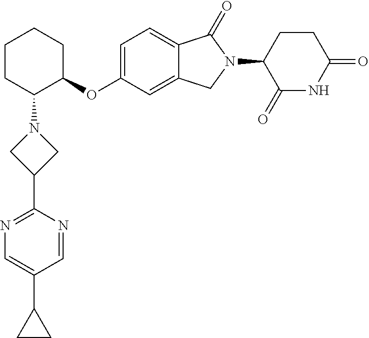 [SMILES: O=C1CC[C@H](N2Cc3cc(O[C@@H]4CCCC[C@H]4N4CC(c5ncc(C6CC6)cn5)C4)ccc3C2=O)C(=O)N1] 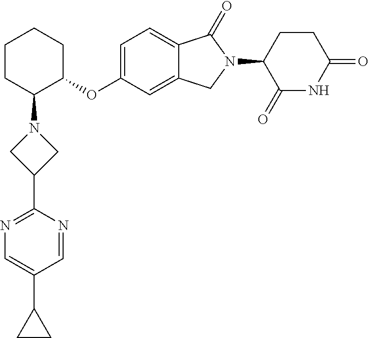 [SMILES: O=C1CC[C@H](N2Cc3cc(O[C@H]4CCCC[C@@H]4N4CC(c5ncc(C6CC6)cn5)C4)ccc3C2=O)C(=O)N1] 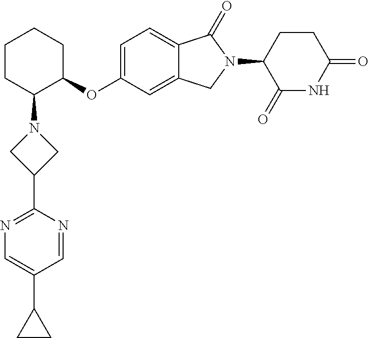 [SMILES: O=C1CC[C@H](N2Cc3cc(O[C@@H]4CCCC[C@@H]4N4CC(c5ncc(C6CC6)cn5)C4)ccc3C2=O)C(=O)N1] 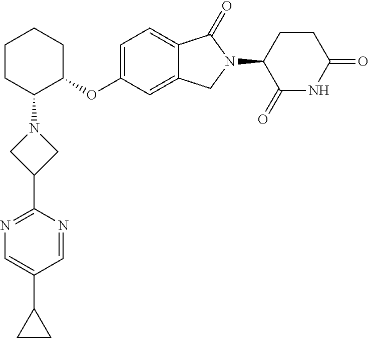 [SMILES: O=C1CC[C@H](N2Cc3cc(O[C@H]4CCCC[C@H]4N4CC(c5ncc(C6CC6)cn5)C4)ccc3C2=O)C(=O)N1] 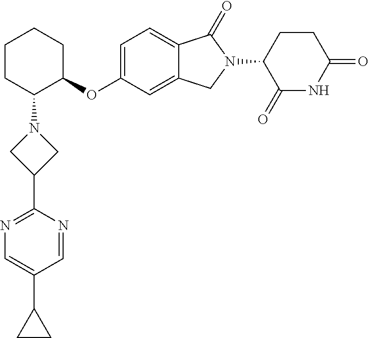 [SMILES: O=C1CC[C@@H](N2Cc3cc(O[C@@H]4CCCC[C@H]4N4CC(c5ncc(C6CC6)cn5)C4)ccc3C2=O)C(=O)N1] 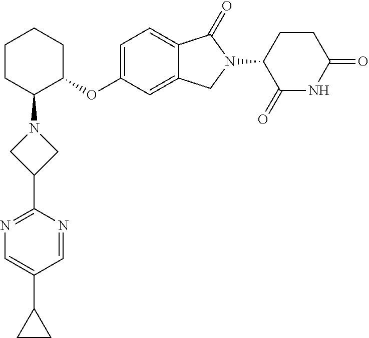 [SMILES: O=C1CC[C@@H](N2Cc3cc(O[C@H]4CCCC[C@@H]4N4CC(c5ncc(C6CC6)cn5)C4)ccc3C2=O)C(=O)N1] 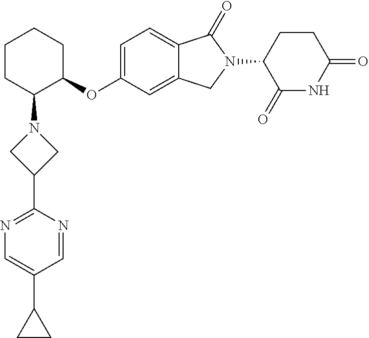 [SMILES: O=C1CC[C@@H](N2Cc3cc(O[C@@H]4CCCC[C@@H]4N4CC(c5ncc(C6CC6)cn5)C4)ccc3C2=O)C(=O)N1] 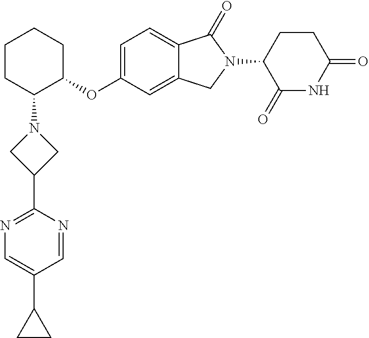 [SMILES: O=C1CC[C@@H](N2Cc3cc(O[C@H]4CCCC[C@H]4N4CC(c5ncc(C6CC6)cn5)C4)ccc3C2=O)C(=O)N1] 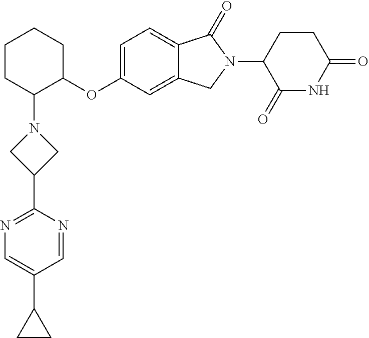 [SMILES: O=C1CCC(N2Cc3cc(OC4CCCCC4N4CC(c5ncc(C6CC6)cn5)C4)ccc3C2=O)C(=O)N1] 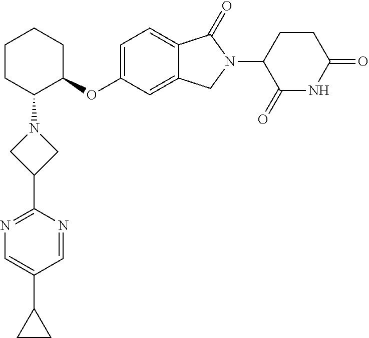 [SMILES: O=C1CCC(N2Cc3cc(O[C@@H]4CCCC[C@H]4N4CC(c5ncc(C6CC6)cn5)C4)ccc3C2=O)C(=O)N1] 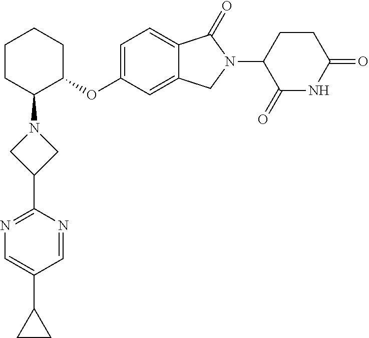 [SMILES: O=C1CCC(N2Cc3cc(O[C@H]4CCCC[C@@H]4N4CC(c5ncc(C6CC6)cn5)C4)ccc3C2=O)C(=O)N1] 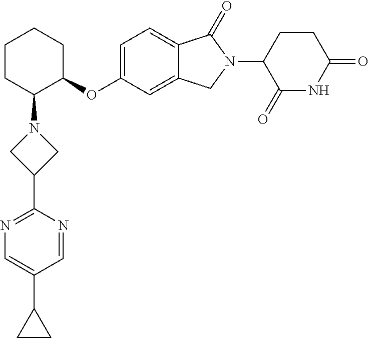 [SMILES: O=C1CCC(N2Cc3cc(O[C@@H]4CCCC[C@@H]4N4CC(c5ncc(C6CC6)cn5)C4)ccc3C2=O)C(=O)N1] 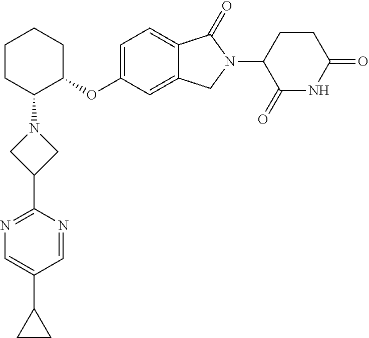 [SMILES: O=C1CCC(N2Cc3cc(O[C@H]4CCCC[C@H]4N4CC(c5ncc(C6CC6)cn5)C4)ccc3C2=O)C(=O)N1] 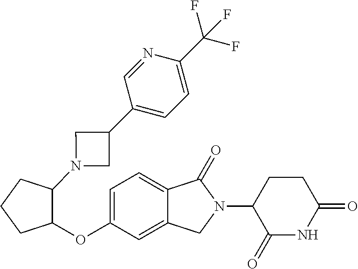 [SMILES: O=C1CCC(N2Cc3cc(OC4CCCC4N4CC(c5ccc(C(F)(F)F)nc5)C4)ccc3C2=O)C(=O)N1] 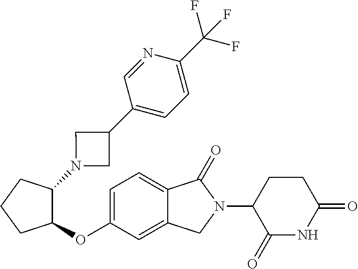 [SMILES: O=C1CCC(N2Cc3cc(O[C@H]4CCC[C@@H]4N4CC(c5ccc(C(F)(F)F)nc5)C4)ccc3C2=O)C(=O)N1] 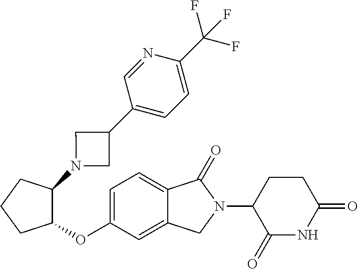 [SMILES: O=C1CCC(N2Cc3cc(O[C@@H]4CCC[C@H]4N4CC(c5ccc(C(F)(F)F)nc5)C4)ccc3C2=O)C(=O)N1] 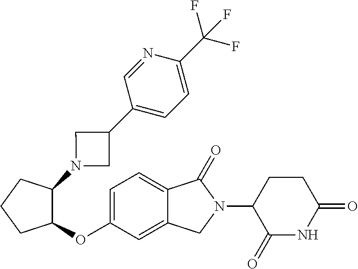 [SMILES: O=C1CCC(N2Cc3cc(O[C@H]4CCC[C@H]4N4CC(c5ccc(C(F)(F)F)nc5)C4)ccc3C2=O)C(=O)N1] 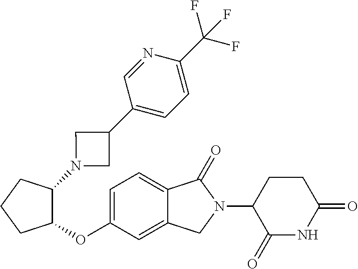 [SMILES: O=C1CCC(N2Cc3cc(O[C@@H]4CCC[C@@H]4N4CC(c5ccc(C(F)(F)F)nc5)C4)ccc3C2=O)C(=O)N1] 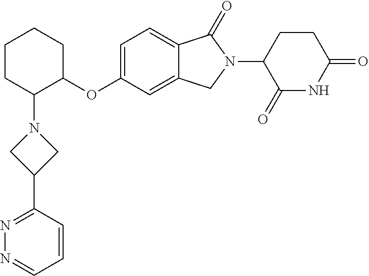 [SMILES: O=C1CCC(N2Cc3cc(OC4CCCCC4N4CC(c5cccnn5)C4)ccc3C2=O)C(=O)N1] 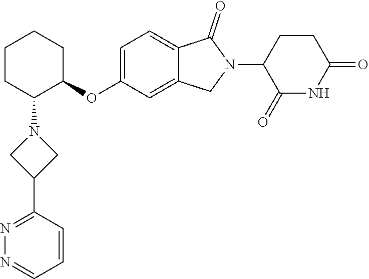 [SMILES: O=C1CCC(N2Cc3cc(O[C@@H]4CCCC[C@H]4N4CC(c5cccnn5)C4)ccc3C2=O)C(=O)N1] 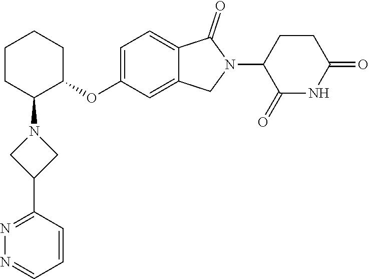 [SMILES: O=C1CCC(N2Cc3cc(O[C@H]4CCCC[C@@H]4N4CC(c5cccnn5)C4)ccc3C2=O)C(=O)N1] 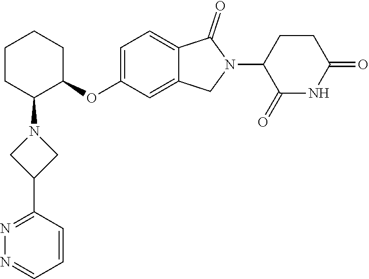 [SMILES: O=C1CCC(N2Cc3cc(O[C@@H]4CCCC[C@@H]4N4CC(c5cccnn5)C4)ccc3C2=O)C(=O)N1] 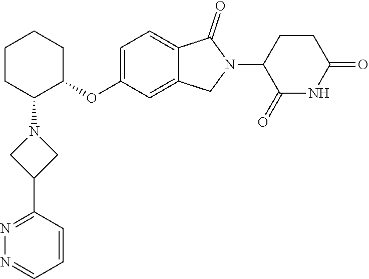 [SMILES: O=C1CCC(N2Cc3cc(O[C@H]4CCCC[C@H]4N4CC(c5cccnn5)C4)ccc3C2=O)C(=O)N1] 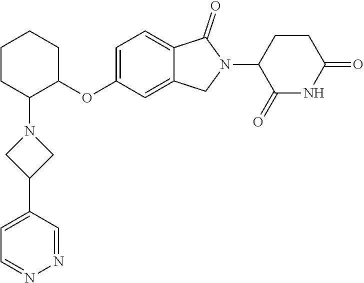 [SMILES: O=C1CCC(N2Cc3cc(OC4CCCCC4N4CC(c5ccnnc5)C4)ccc3C2=O)C(=O)N1] 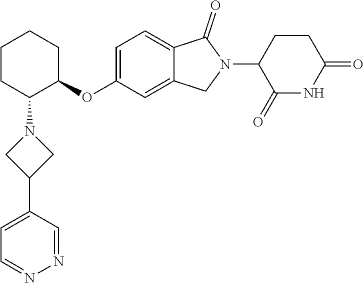 [SMILES: O=C1CCC(N2Cc3cc(O[C@@H]4CCCC[C@H]4N4CC(c5ccnnc5)C4)ccc3C2=O)C(=O)N1] 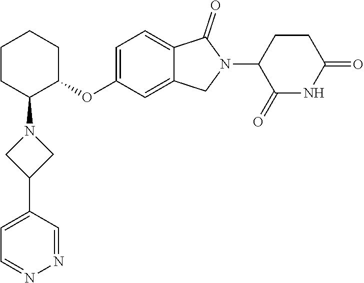 [SMILES: O=C1CCC(N2Cc3cc(O[C@H]4CCCC[C@@H]4N4CC(c5ccnnc5)C4)ccc3C2=O)C(=O)N1] 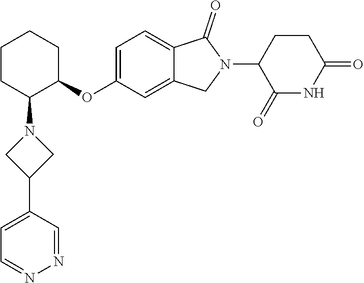 [SMILES: O=C1CCC(N2Cc3cc(O[C@@H]4CCCC[C@@H]4N4CC(c5ccnnc5)C4)ccc3C2=O)C(=O)N1] 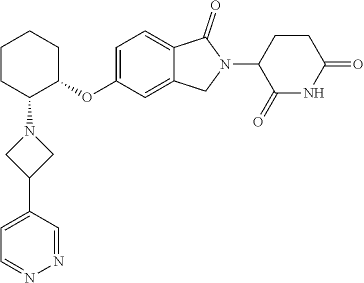 [SMILES: O=C1CCC(N2Cc3cc(O[C@H]4CCCC[C@H]4N4CC(c5ccnnc5)C4)ccc3C2=O)C(=O)N1] 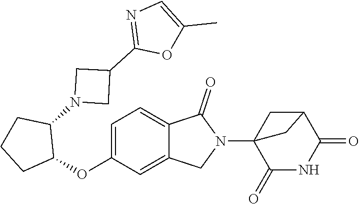 [SMILES: Cc1cnc(C2CN([C@H]3CCC[C@H]3Oc3ccc4c(c3)CN(C35CC(C3)C(=O)NC5=O)C4=O)C2)o1] 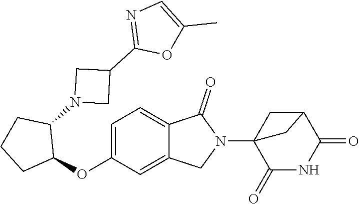 [SMILES: Cc1cnc(C2CN([C@H]3CCC[C@@H]3Oc3ccc4c(c3)CN(C35CC(C3)C(=O)NC5=O)C4=O)C2)o1] 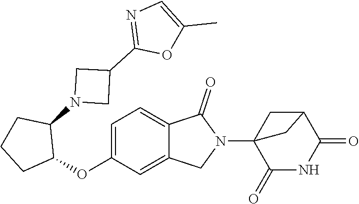 [SMILES: Cc1cnc(C2CN([C@@H]3CCC[C@H]3Oc3ccc4c(c3)CN(C35CC(C3)C(=O)NC5=O)C4=O)C2)o1] 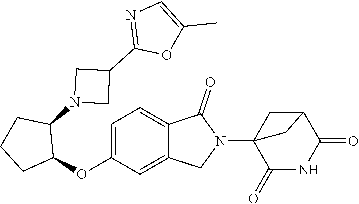 [SMILES: Cc1cnc(C2CN([C@@H]3CCC[C@@H]3Oc3ccc4c(c3)CN(C35CC(C3)C(=O)NC5=O)C4=O)C2)o1] 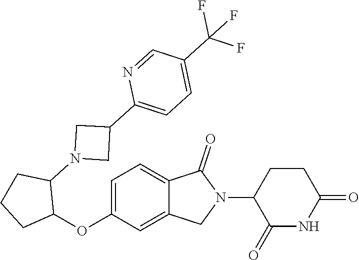 [SMILES: O=C1CCC(N2Cc3cc(OC4CCCC4N4CC(c5ccc(C(F)(F)F)cn5)C4)ccc3C2=O)C(=O)N1] 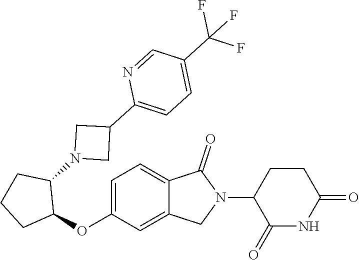 [SMILES: O=C1CCC(N2Cc3cc(O[C@H]4CCC[C@@H]4N4CC(c5ccc(C(F)(F)F)cn5)C4)ccc3C2=O)C(=O)N1] 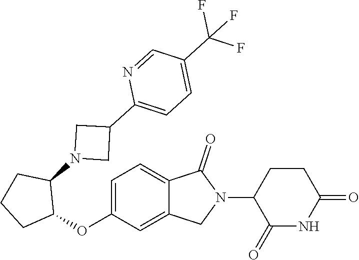 [SMILES: O=C1CCC(N2Cc3cc(O[C@@H]4CCC[C@H]4N4CC(c5ccc(C(F)(F)F)cn5)C4)ccc3C2=O)C(=O)N1] 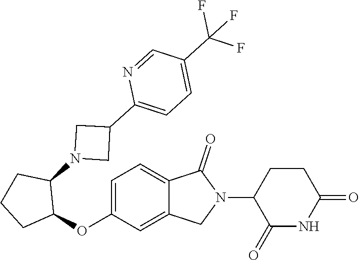 [SMILES: O=C1CCC(N2Cc3cc(O[C@H]4CCC[C@H]4N4CC(c5ccc(C(F)(F)F)cn5)C4)ccc3C2=O)C(=O)N1] 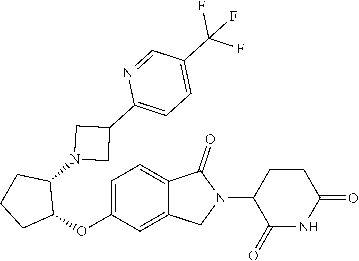 [SMILES: O=C1CCC(N2Cc3cc(O[C@@H]4CCC[C@@H]4N4CC(c5ccc(C(F)(F)F)cn5)C4)ccc3C2=O)C(=O)N1] 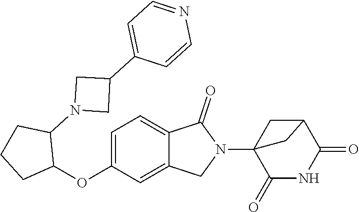 [SMILES: O=C1NC(=O)C2(N3Cc4cc(OC5CCCC5N5CC(c6ccncc6)C5)ccc4C3=O)CC1C2] 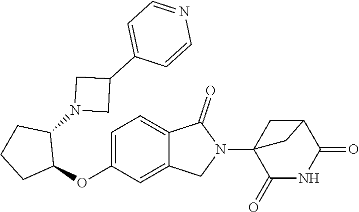 [SMILES: O=C1NC(=O)C2(N3Cc4cc(O[C@H]5CCC[C@@H]5N5CC(c6ccncc6)C5)ccc4C3=O)CC1C2] 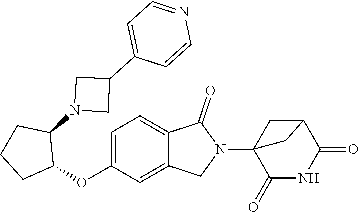 [SMILES: O=C1NC(=O)C2(N3Cc4cc(O[C@@H]5CCC[C@H]5N5CC(c6ccncc6)C5)ccc4C3=O)CC1C2] 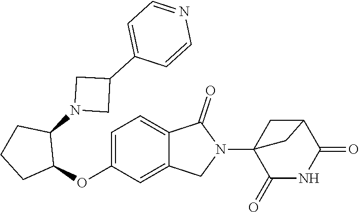 [SMILES: O=C1NC(=O)C2(N3Cc4cc(O[C@H]5CCC[C@H]5N5CC(c6ccncc6)C5)ccc4C3=O)CC1C2] 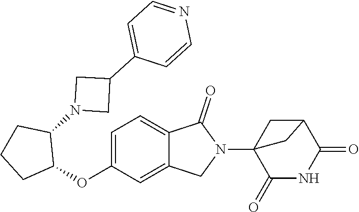 [SMILES: O=C1NC(=O)C2(N3Cc4cc(O[C@@H]5CCC[C@@H]5N5CC(c6ccncc6)C5)ccc4C3=O)CC1C2] 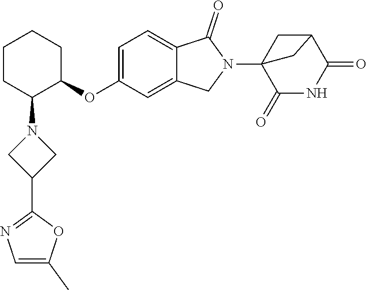 [SMILES: Cc1cnc(C2CN([C@H]3CCCC[C@H]3Oc3ccc4c(c3)CN(C35CC(C3)C(=O)NC5=O)C4=O)C2)o1] 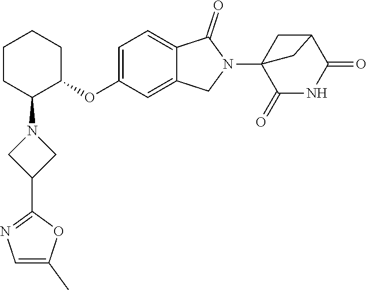 [SMILES: Cc1cnc(C2CN([C@H]3CCCC[C@@H]3Oc3ccc4c(c3)CN(C35CC(C3)C(=O)NC5=O)C4=O)C2)o1] 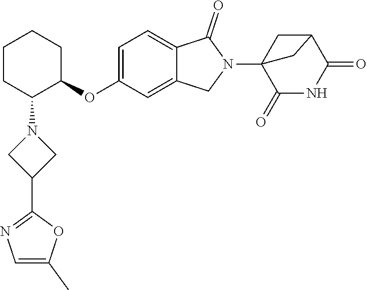 [SMILES: Cc1cnc(C2CN([C@@H]3CCCC[C@H]3Oc3ccc4c(c3)CN(C35CC(C3)C(=O)NC5=O)C4=O)C2)o1] 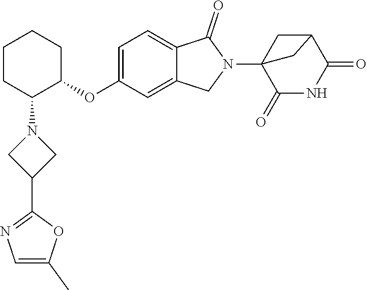 [SMILES: Cc1cnc(C2CN([C@@H]3CCCC[C@@H]3Oc3ccc4c(c3)CN(C35CC(C3)C(=O)NC5=O)C4=O)C2)o1] 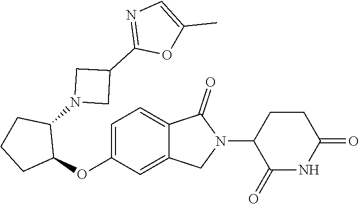 [SMILES: Cc1cnc(C2CN([C@H]3CCC[C@@H]3Oc3ccc4c(c3)CN(C3CCC(=O)NC3=O)C4=O)C2)o1] 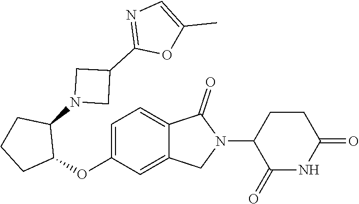 [SMILES: Cc1cnc(C2CN([C@@H]3CCC[C@H]3Oc3ccc4c(c3)CN(C3CCC(=O)NC3=O)C4=O)C2)o1] 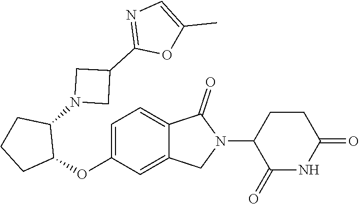 [SMILES: Cc1cnc(C2CN([C@H]3CCC[C@H]3Oc3ccc4c(c3)CN(C3CCC(=O)NC3=O)C4=O)C2)o1] 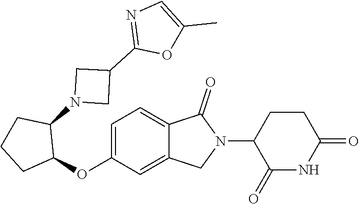 [SMILES: Cc1cnc(C2CN([C@@H]3CCC[C@@H]3Oc3ccc4c(c3)CN(C3CCC(=O)NC3=O)C4=O)C2)o1] 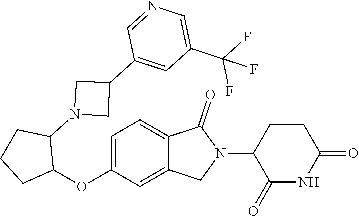 [SMILES: O=C1CCC(N2Cc3cc(OC4CCCC4N4CC(c5cncc(C(F)(F)F)c5)C4)ccc3C2=O)C(=O)N1] 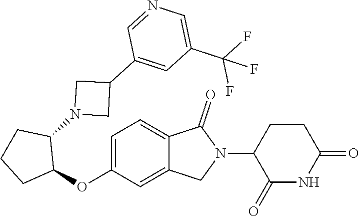 [SMILES: O=C1CCC(N2Cc3cc(O[C@H]4CCC[C@@H]4N4CC(c5cncc(C(F)(F)F)c5)C4)ccc3C2=O)C(=O)N1] 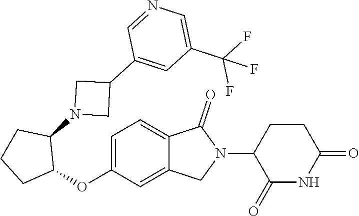 [SMILES: O=C1CCC(N2Cc3cc(O[C@@H]4CCC[C@H]4N4CC(c5cncc(C(F)(F)F)c5)C4)ccc3C2=O)C(=O)N1] 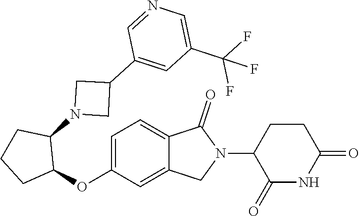 [SMILES: O=C1CCC(N2Cc3cc(O[C@H]4CCC[C@H]4N4CC(c5cncc(C(F)(F)F)c5)C4)ccc3C2=O)C(=O)N1] 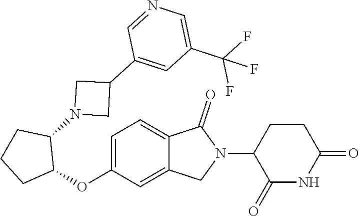 [SMILES: O=C1CCC(N2Cc3cc(O[C@@H]4CCC[C@@H]4N4CC(c5cncc(C(F)(F)F)c5)C4)ccc3C2=O)C(=O)N1] 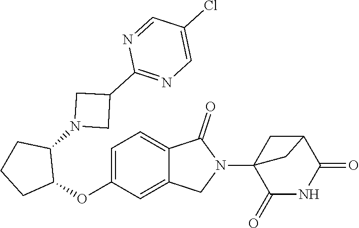 [SMILES: O=C1NC(=O)C2(N3Cc4cc(O[C@@H]5CCC[C@@H]5N5CC(c6ncc(Cl)cn6)C5)ccc4C3=O)CC1C2] 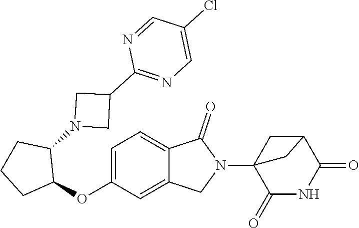 [SMILES: O=C1NC(=O)C2(N3Cc4cc(O[C@H]5CCC[C@@H]5N5CC(c6ncc(Cl)cn6)C5)ccc4C3=O)CC1C2] 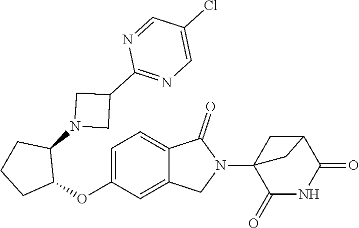 [SMILES: O=C1NC(=O)C2(N3Cc4cc(O[C@@H]5CCC[C@H]5N5CC(c6ncc(Cl)cn6)C5)ccc4C3=O)CC1C2] 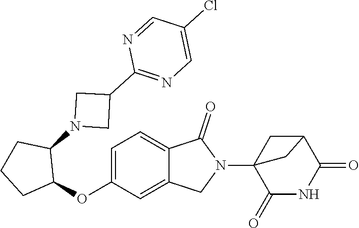 [SMILES: O=C1NC(=O)C2(N3Cc4cc(O[C@H]5CCC[C@H]5N5CC(c6ncc(Cl)cn6)C5)ccc4C3=O)CC1C2] 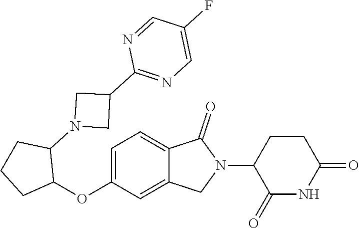 [SMILES: O=C1CCC(N2Cc3cc(OC4CCCC4N4CC(c5ncc(F)cn5)C4)ccc3C2=O)C(=O)N1] 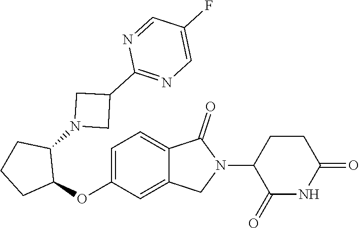 [SMILES: O=C1CCC(N2Cc3cc(O[C@H]4CCC[C@@H]4N4CC(c5ncc(F)cn5)C4)ccc3C2=O)C(=O)N1] 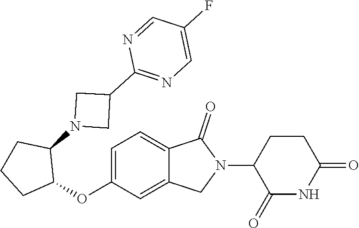 [SMILES: O=C1CCC(N2Cc3cc(O[C@@H]4CCC[C@H]4N4CC(c5ncc(F)cn5)C4)ccc3C2=O)C(=O)N1] 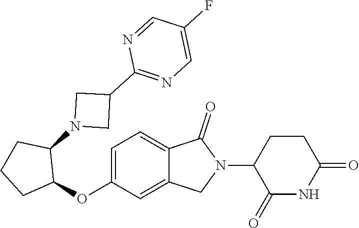 [SMILES: O=C1CCC(N2Cc3cc(O[C@H]4CCC[C@H]4N4CC(c5ncc(F)cn5)C4)ccc3C2=O)C(=O)N1] 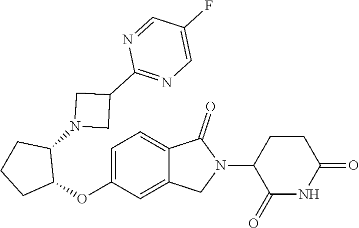 [SMILES: O=C1CCC(N2Cc3cc(O[C@@H]4CCC[C@@H]4N4CC(c5ncc(F)cn5)C4)ccc3C2=O)C(=O)N1] 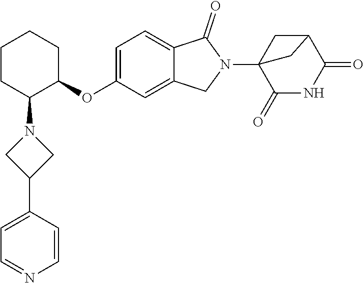 [SMILES: O=C1NC(=O)C2(N3Cc4cc(O[C@@H]5CCCC[C@@H]5N5CC(c6ccncc6)C5)ccc4C3=O)CC1C2] 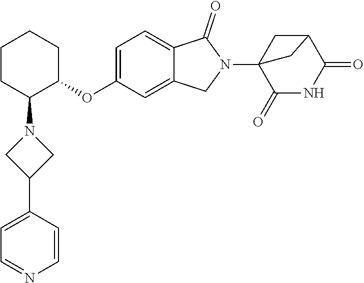 [SMILES: O=C1NC(=O)C2(N3Cc4cc(O[C@H]5CCCC[C@@H]5N5CC(c6ccncc6)C5)ccc4C3=O)CC1C2] 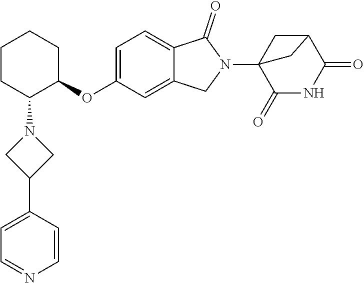 [SMILES: O=C1NC(=O)C2(N3Cc4cc(O[C@@H]5CCCC[C@H]5N5CC(c6ccncc6)C5)ccc4C3=O)CC1C2] 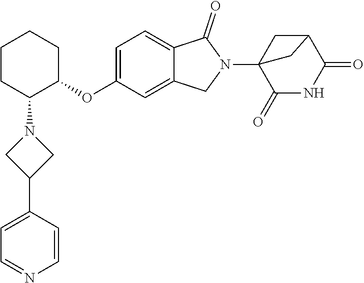 [SMILES: O=C1NC(=O)C2(N3Cc4cc(O[C@H]5CCCC[C@H]5N5CC(c6ccncc6)C5)ccc4C3=O)CC1C2] 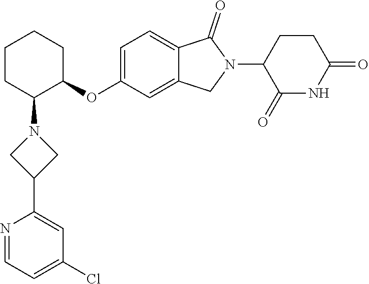 [SMILES: O=C1CCC(N2Cc3cc(O[C@@H]4CCCC[C@@H]4N4CC(c5cc(Cl)ccn5)C4)ccc3C2=O)C(=O)N1] 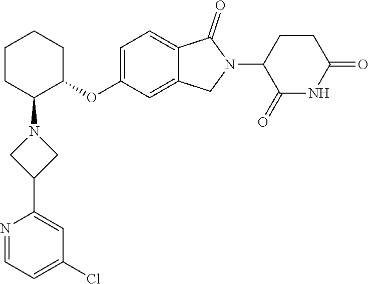 [SMILES: O=C1CCC(N2Cc3cc(O[C@H]4CCCC[C@@H]4N4CC(c5cc(Cl)ccn5)C4)ccc3C2=O)C(=O)N1] 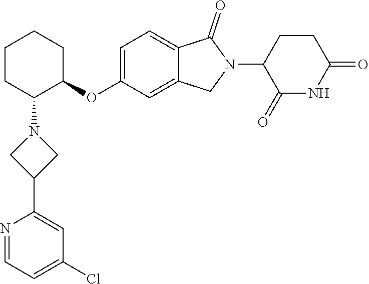 [SMILES: O=C1CCC(N2Cc3cc(O[C@@H]4CCCC[C@H]4N4CC(c5cc(Cl)ccn5)C4)ccc3C2=O)C(=O)N1] 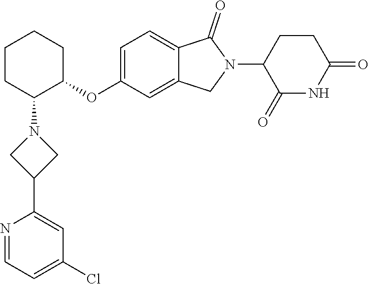 [SMILES: O=C1CCC(N2Cc3cc(O[C@H]4CCCC[C@H]4N4CC(c5cc(Cl)ccn5)C4)ccc3C2=O)C(=O)N1] 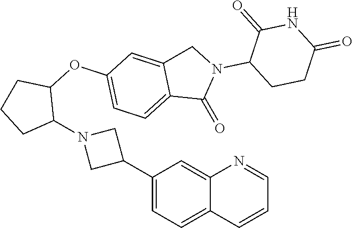 [SMILES: O=C1CCC(N2Cc3cc(OC4CCCC4N4CC(c5ccc6cccnc6c5)C4)ccc3C2=O)C(=O)N1] 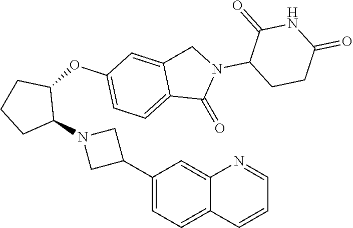 [SMILES: O=C1CCC(N2Cc3cc(O[C@H]4CCC[C@@H]4N4CC(c5ccc6cccnc6c5)C4)ccc3C2=O)C(=O)N1] 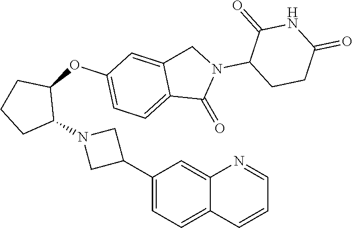 [SMILES: O=C1CCC(N2Cc3cc(O[C@@H]4CCC[C@H]4N4CC(c5ccc6cccnc6c5)C4)ccc3C2=O)C(=O)N1] 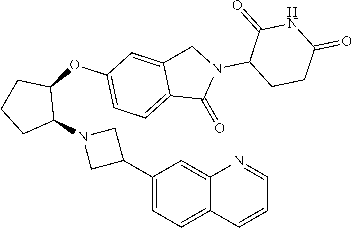 [SMILES: O=C1CCC(N2Cc3cc(O[C@@H]4CCC[C@@H]4N4CC(c5ccc6cccnc6c5)C4)ccc3C2=O)C(=O)N1] 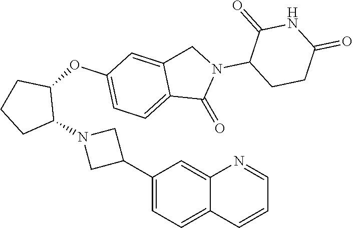 [SMILES: O=C1CCC(N2Cc3cc(O[C@H]4CCC[C@H]4N4CC(c5ccc6cccnc6c5)C4)ccc3C2=O)C(=O)N1] 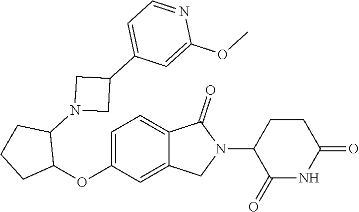 [SMILES: COc1cc(C2CN(C3CCCC3Oc3ccc4c(c3)CN(C3CCC(=O)NC3=O)C4=O)C2)ccn1] 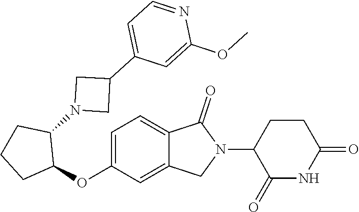 [SMILES: COc1cc(C2CN([C@H]3CCC[C@@H]3Oc3ccc4c(c3)CN(C3CCC(=O)NC3=O)C4=O)C2)ccn1] 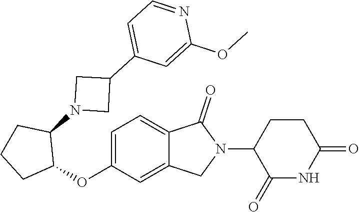 [SMILES: COc1cc(C2CN([C@@H]3CCC[C@H]3Oc3ccc4c(c3)CN(C3CCC(=O)NC3=O)C4=O)C2)ccn1] 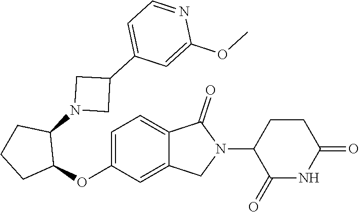 [SMILES: COc1cc(C2CN([C@@H]3CCC[C@@H]3Oc3ccc4c(c3)CN(C3CCC(=O)NC3=O)C4=O)C2)ccn1] 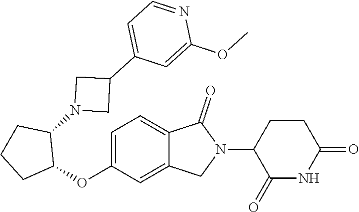 [SMILES: COc1cc(C2CN([C@H]3CCC[C@H]3Oc3ccc4c(c3)CN(C3CCC(=O)NC3=O)C4=O)C2)ccn1] 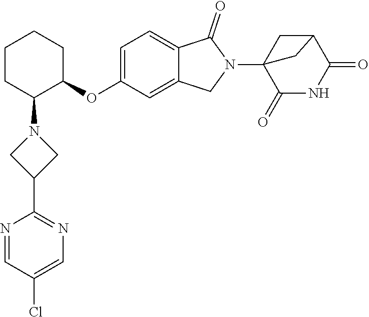 [SMILES: O=C1NC(=O)C2(N3Cc4cc(O[C@@H]5CCCC[C@@H]5N5CC(c6ncc(Cl)cn6)C5)ccc4C3=O)CC1C2] 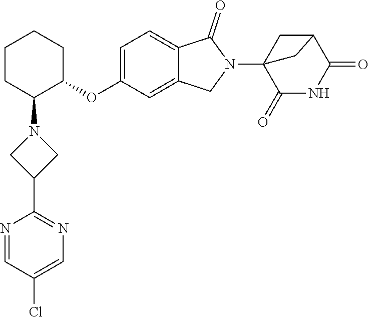 [SMILES: O=C1NC(=O)C2(N3Cc4cc(O[C@H]5CCCC[C@@H]5N5CC(c6ncc(Cl)cn6)C5)ccc4C3=O)CC1C2] 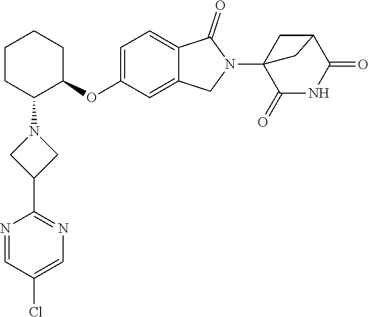 [SMILES: O=C1NC(=O)C2(N3Cc4cc(O[C@@H]5CCCC[C@H]5N5CC(c6ncc(Cl)cn6)C5)ccc4C3=O)CC1C2] 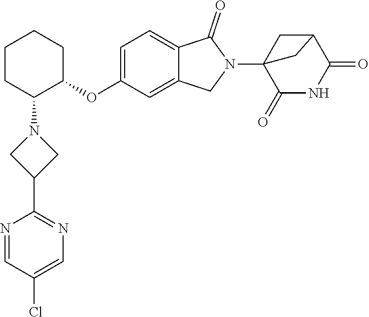 [SMILES: O=C1NC(=O)C2(N3Cc4cc(O[C@H]5CCCC[C@H]5N5CC(c6ncc(Cl)cn6)C5)ccc4C3=O)CC1C2] 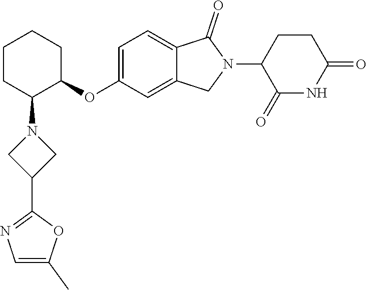 [SMILES: Cc1cnc(C2CN([C@H]3CCCC[C@H]3Oc3ccc4c(c3)CN(C3CCC(=O)NC3=O)C4=O)C2)o1] 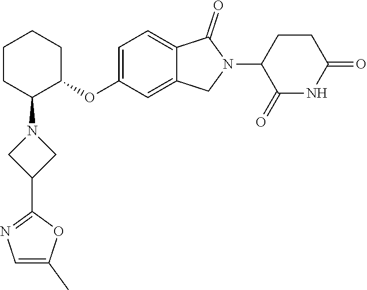 [SMILES: Cc1cnc(C2CN([C@H]3CCCC[C@@H]3Oc3ccc4c(c3)CN(C3CCC(=O)NC3=O)C4=O)C2)o1] 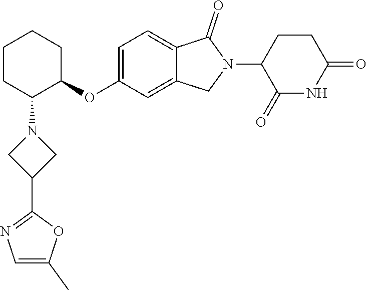 [SMILES: Cc1cnc(C2CN([C@@H]3CCCC[C@H]3Oc3ccc4c(c3)CN(C3CCC(=O)NC3=O)C4=O)C2)o1] 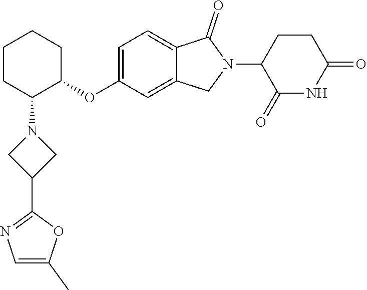 [SMILES: Cc1cnc(C2CN([C@@H]3CCCC[C@@H]3Oc3ccc4c(c3)CN(C3CCC(=O)NC3=O)C4=O)C2)o1] 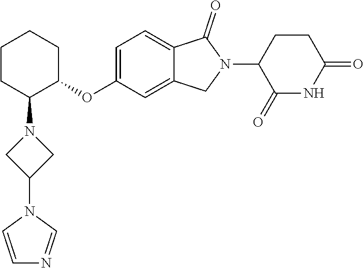 [SMILES: O=C1CCC(N2Cc3cc(O[C@H]4CCCC[C@@H]4N4CC(n5ccnc5)C4)ccc3C2=O)C(=O)N1] 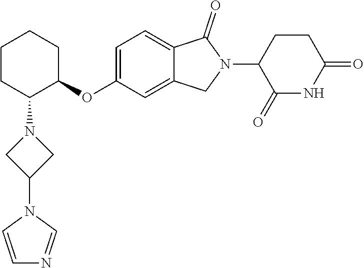 [SMILES: O=C1CCC(N2Cc3cc(O[C@@H]4CCCC[C@H]4N4CC(n5ccnc5)C4)ccc3C2=O)C(=O)N1] 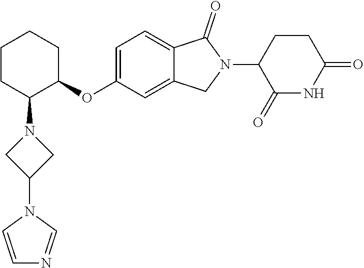 [SMILES: O=C1CCC(N2Cc3cc(O[C@@H]4CCCC[C@@H]4N4CC(n5ccnc5)C4)ccc3C2=O)C(=O)N1] 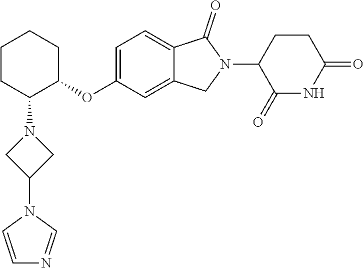 [SMILES: O=C1CCC(N2Cc3cc(O[C@H]4CCCC[C@H]4N4CC(n5ccnc5)C4)ccc3C2=O)C(=O)N1] 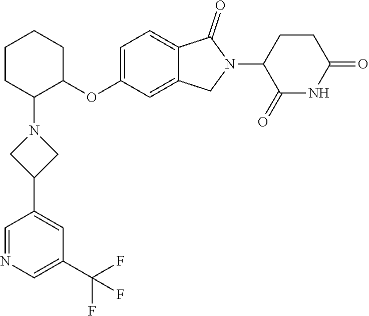 [SMILES: O=C1CCC(N2Cc3cc(OC4CCCCC4N4CC(c5cncc(C(F)(F)F)c5)C4)ccc3C2=O)C(=O)N1] 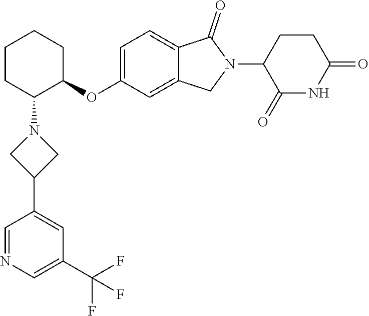 [SMILES: O=C1CCC(N2Cc3cc(O[C@@H]4CCCC[C@H]4N4CC(c5cncc(C(F)(F)F)c5)C4)ccc3C2=O)C(=O)N1] 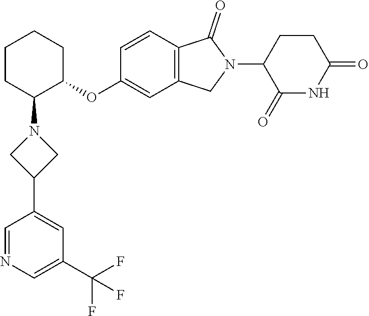 [SMILES: O=C1CCC(N2Cc3cc(O[C@H]4CCCC[C@@H]4N4CC(c5cncc(C(F)(F)F)c5)C4)ccc3C2=O)C(=O)N1] 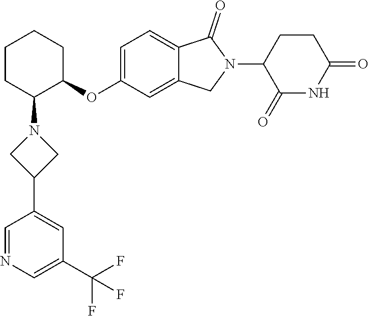 [SMILES: O=C1CCC(N2Cc3cc(O[C@@H]4CCCC[C@@H]4N4CC(c5cncc(C(F)(F)F)c5)C4)ccc3C2=O)C(=O)N1] 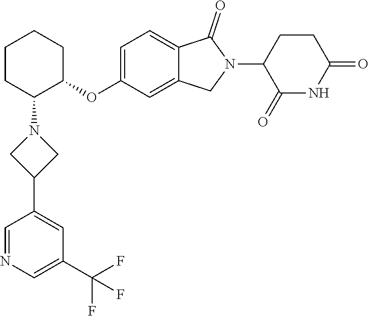 [SMILES: O=C1CCC(N2Cc3cc(O[C@H]4CCCC[C@H]4N4CC(c5cncc(C(F)(F)F)c5)C4)ccc3C2=O)C(=O)N1] 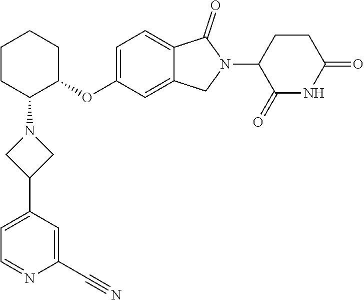 [SMILES: N#Cc1cc(C2CN([C@@H]3CCCC[C@@H]3Oc3ccc4c(c3)CN(C3CCC(=O)NC3=O)C4=O)C2)ccn1] 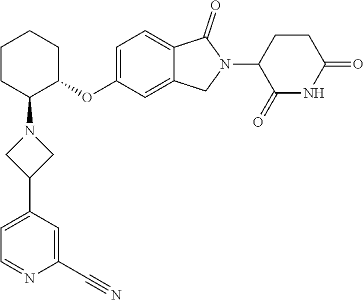 [SMILES: N#Cc1cc(C2CN([C@H]3CCCC[C@@H]3Oc3ccc4c(c3)CN(C3CCC(=O)NC3=O)C4=O)C2)ccn1] 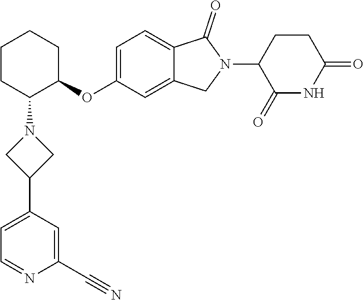 [SMILES: N#Cc1cc(C2CN([C@@H]3CCCC[C@H]3Oc3ccc4c(c3)CN(C3CCC(=O)NC3=O)C4=O)C2)ccn1] 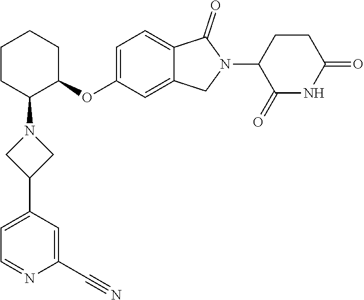 [SMILES: N#Cc1cc(C2CN([C@H]3CCCC[C@H]3Oc3ccc4c(c3)CN(C3CCC(=O)NC3=O)C4=O)C2)ccn1] 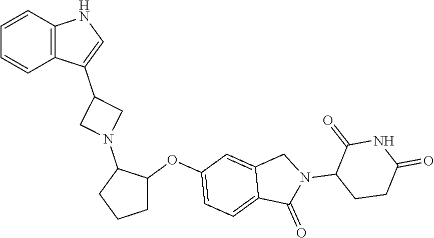 [SMILES: O=C1CCC(N2Cc3cc(OC4CCCC4N4CC(c5c[nH]c6ccccc56)C4)ccc3C2=O)C(=O)N1] 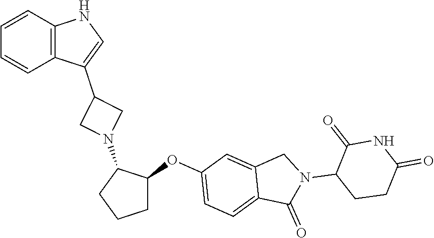 [SMILES: O=C1CCC(N2Cc3cc(O[C@H]4CCC[C@@H]4N4CC(c5c[nH]c6ccccc56)C4)ccc3C2=O)C(=O)N1] 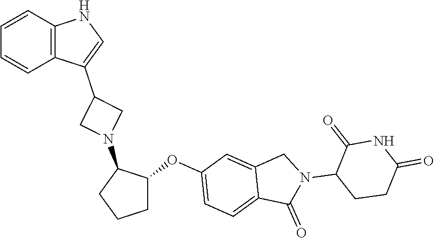 [SMILES: O=C1CCC(N2Cc3cc(O[C@@H]4CCC[C@H]4N4CC(c5c[nH]c6ccccc56)C4)ccc3C2=O)C(=O)N1] 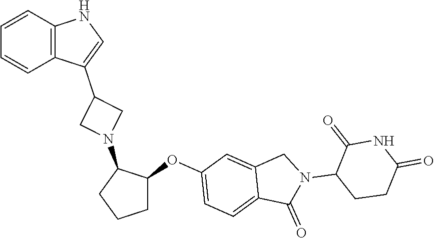 [SMILES: O=C1CCC(N2Cc3cc(O[C@H]4CCC[C@H]4N4CC(c5c[nH]c6ccccc56)C4)ccc3C2=O)C(=O)N1] 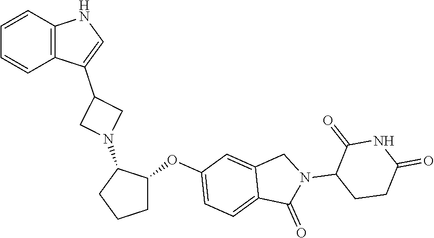 [SMILES: O=C1CCC(N2Cc3cc(O[C@@H]4CCC[C@@H]4N4CC(c5c[nH]c6ccccc56)C4)ccc3C2=O)C(=O)N1] 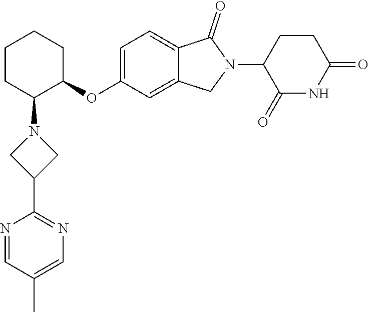 [SMILES: Cc1cnc(C2CN([C@H]3CCCC[C@H]3Oc3ccc4c(c3)CN(C3CCC(=O)NC3=O)C4=O)C2)nc1] 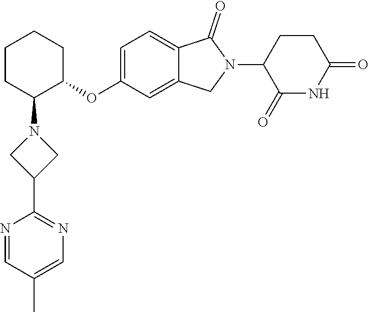 [SMILES: Cc1cnc(C2CN([C@H]3CCCC[C@@H]3Oc3ccc4c(c3)CN(C3CCC(=O)NC3=O)C4=O)C2)nc1] 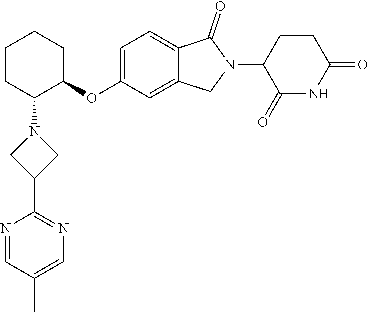 [SMILES: Cc1cnc(C2CN([C@@H]3CCCC[C@H]3Oc3ccc4c(c3)CN(C3CCC(=O)NC3=O)C4=O)C2)nc1] 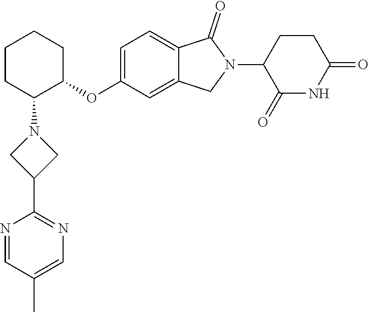 [SMILES: Cc1cnc(C2CN([C@@H]3CCCC[C@@H]3Oc3ccc4c(c3)CN(C3CCC(=O)NC3=O)C4=O)C2)nc1] 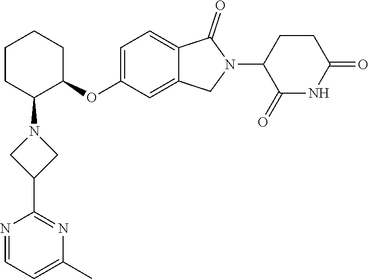 [SMILES: Cc1ccnc(C2CN([C@H]3CCCC[C@H]3Oc3ccc4c(c3)CN(C3CCC(=O)NC3=O)C4=O)C2)n1] 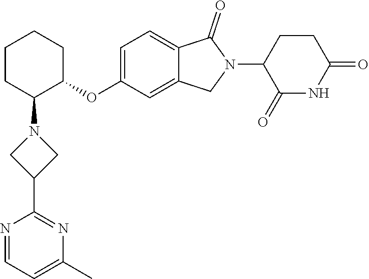 [SMILES: Cc1ccnc(C2CN([C@H]3CCCC[C@@H]3Oc3ccc4c(c3)CN(C3CCC(=O)NC3=O)C4=O)C2)n1] 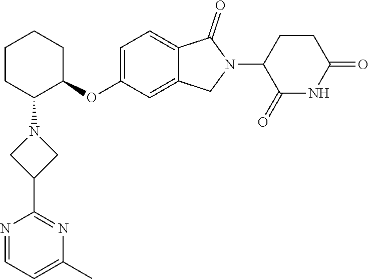 [SMILES: Cc1ccnc(C2CN([C@@H]3CCCC[C@H]3Oc3ccc4c(c3)CN(C3CCC(=O)NC3=O)C4=O)C2)n1] 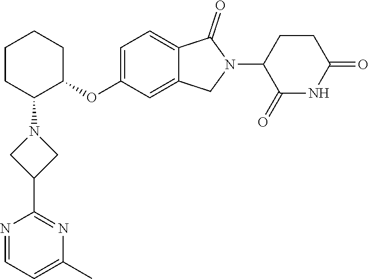 [SMILES: Cc1ccnc(C2CN([C@@H]3CCCC[C@@H]3Oc3ccc4c(c3)CN(C3CCC(=O)NC3=O)C4=O)C2)n1] 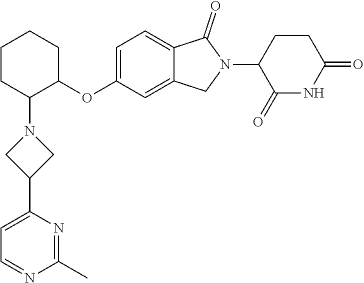 [SMILES: Cc1nccc(C2CN(C3CCCCC3Oc3ccc4c(c3)CN(C3CCC(=O)NC3=O)C4=O)C2)n1] 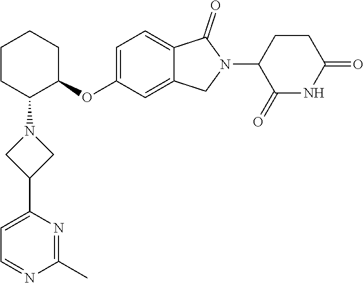 [SMILES: Cc1nccc(C2CN([C@@H]3CCCC[C@H]3Oc3ccc4c(c3)CN(C3CCC(=O)NC3=O)C4=O)C2)n1] 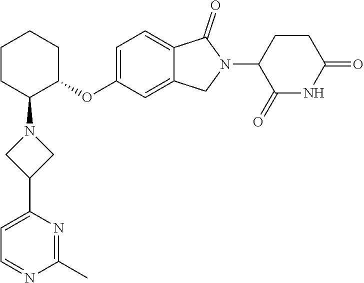 [SMILES: Cc1nccc(C2CN([C@H]3CCCC[C@@H]3Oc3ccc4c(c3)CN(C3CCC(=O)NC3=O)C4=O)C2)n1] 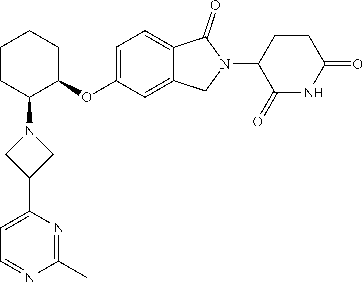 [SMILES: Cc1nccc(C2CN([C@H]3CCCC[C@H]3Oc3ccc4c(c3)CN(C3CCC(=O)NC3=O)C4=O)C2)n1] 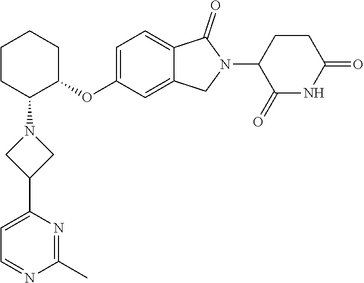 [SMILES: Cc1nccc(C2CN([C@@H]3CCCC[C@@H]3Oc3ccc4c(c3)CN(C3CCC(=O)NC3=O)C4=O)C2)n1] 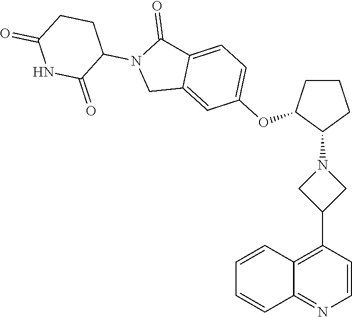 [SMILES: O=C1CCC(N2Cc3cc(O[C@@H]4CCC[C@@H]4N4CC(c5ccnc6ccccc56)C4)ccc3C2=O)C(=O)N1] 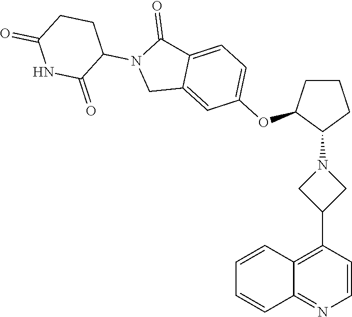 [SMILES: O=C1CCC(N2Cc3cc(O[C@H]4CCC[C@@H]4N4CC(c5ccnc6ccccc56)C4)ccc3C2=O)C(=O)N1] 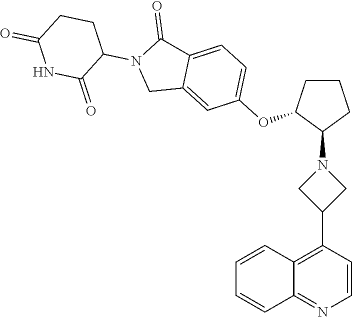 [SMILES: O=C1CCC(N2Cc3cc(O[C@@H]4CCC[C@H]4N4CC(c5ccnc6ccccc56)C4)ccc3C2=O)C(=O)N1] 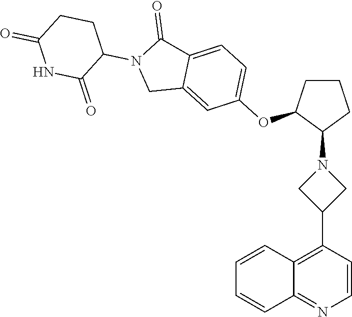 [SMILES: O=C1CCC(N2Cc3cc(O[C@H]4CCC[C@H]4N4CC(c5ccnc6ccccc56)C4)ccc3C2=O)C(=O)N1] 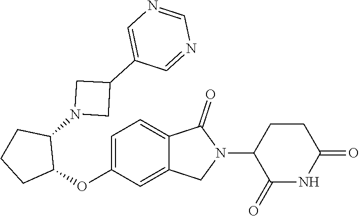 [SMILES: O=C1CCC(N2Cc3cc(O[C@@H]4CCC[C@@H]4N4CC(c5cncnc5)C4)ccc3C2=O)C(=O)N1] 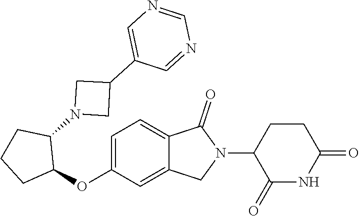 [SMILES: O=C1CCC(N2Cc3cc(O[C@H]4CCC[C@@H]4N4CC(c5cncnc5)C4)ccc3C2=O)C(=O)N1] 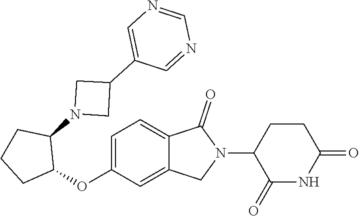 [SMILES: O=C1CCC(N2Cc3cc(O[C@@H]4CCC[C@H]4N4CC(c5cncnc5)C4)ccc3C2=O)C(=O)N1] 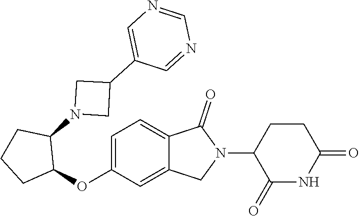 [SMILES: O=C1CCC(N2Cc3cc(O[C@H]4CCC[C@H]4N4CC(c5cncnc5)C4)ccc3C2=O)C(=O)N1] 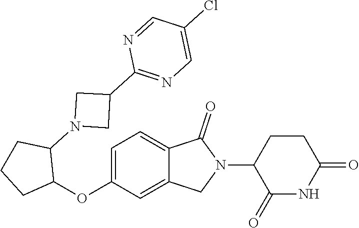 [SMILES: O=C1CCC(N2Cc3cc(OC4CCCC4N4CC(c5ncc(Cl)cn5)C4)ccc3C2=O)C(=O)N1] 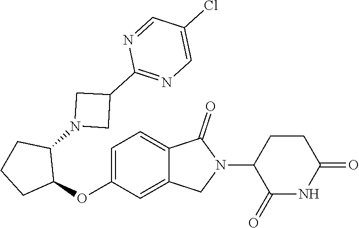 [SMILES: O=C1CCC(N2Cc3cc(O[C@H]4CCC[C@@H]4N4CC(c5ncc(Cl)cn5)C4)ccc3C2=O)C(=O)N1] 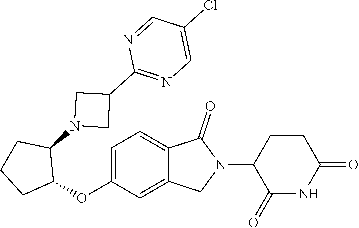 [SMILES: O=C1CCC(N2Cc3cc(O[C@@H]4CCC[C@H]4N4CC(c5ncc(Cl)cn5)C4)ccc3C2=O)C(=O)N1] 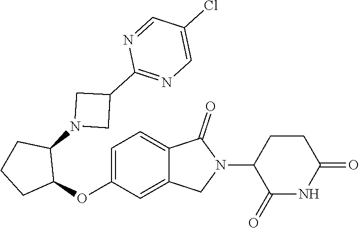 [SMILES: O=C1CCC(N2Cc3cc(O[C@H]4CCC[C@H]4N4CC(c5ncc(Cl)cn5)C4)ccc3C2=O)C(=O)N1] 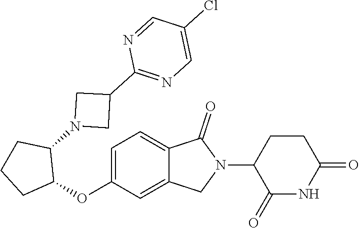 [SMILES: O=C1CCC(N2Cc3cc(O[C@@H]4CCC[C@@H]4N4CC(c5ncc(Cl)cn5)C4)ccc3C2=O)C(=O)N1] 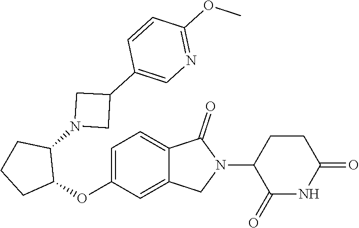 [SMILES: COc1ccc(C2CN([C@H]3CCC[C@H]3Oc3ccc4c(c3)CN(C3CCC(=O)NC3=O)C4=O)C2)cn1] 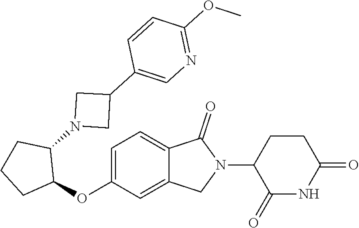 [SMILES: COc1ccc(C2CN([C@H]3CCC[C@@H]3Oc3ccc4c(c3)CN(C3CCC(=O)NC3=O)C4=O)C2)cn1] 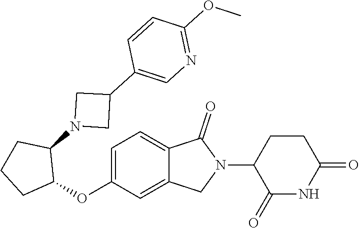 [SMILES: COc1ccc(C2CN([C@@H]3CCC[C@H]3Oc3ccc4c(c3)CN(C3CCC(=O)NC3=O)C4=O)C2)cn1] 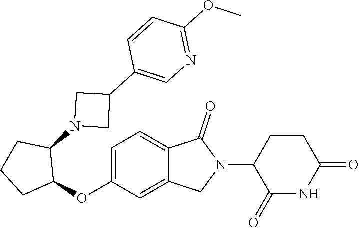 [SMILES: COc1ccc(C2CN([C@@H]3CCC[C@@H]3Oc3ccc4c(c3)CN(C3CCC(=O)NC3=O)C4=O)C2)cn1] 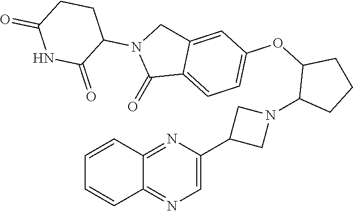 [SMILES: O=C1CCC(N2Cc3cc(OC4CCCC4N4CC(c5cnc6ccccc6n5)C4)ccc3C2=O)C(=O)N1] 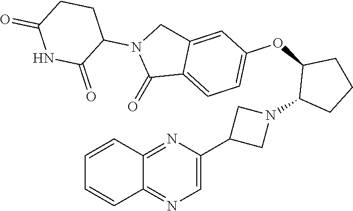 [SMILES: O=C1CCC(N2Cc3cc(O[C@H]4CCC[C@@H]4N4CC(c5cnc6ccccc6n5)C4)ccc3C2=O)C(=O)N1] 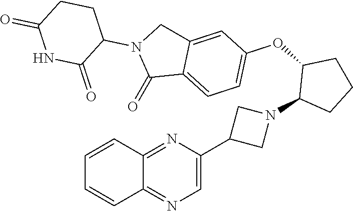 [SMILES: O=C1CCC(N2Cc3cc(O[C@@H]4CCC[C@H]4N4CC(c5cnc6ccccc6n5)C4)ccc3C2=O)C(=O)N1] 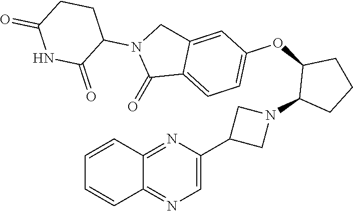 [SMILES: O=C1CCC(N2Cc3cc(O[C@H]4CCC[C@H]4N4CC(c5cnc6ccccc6n5)C4)ccc3C2=O)C(=O)N1] 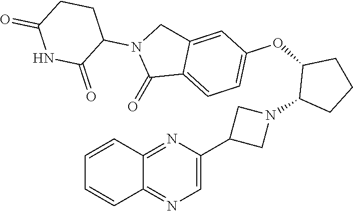 [SMILES: O=C1CCC(N2Cc3cc(O[C@@H]4CCC[C@@H]4N4CC(c5cnc6ccccc6n5)C4)ccc3C2=O)C(=O)N1] 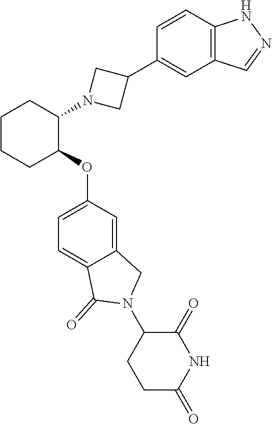 [SMILES: O=C1CCC(N2Cc3cc(O[C@H]4CCCC[C@@H]4N4CC(c5ccc6[nH]ncc6c5)C4)ccc3C2=O)C(=O)N1] 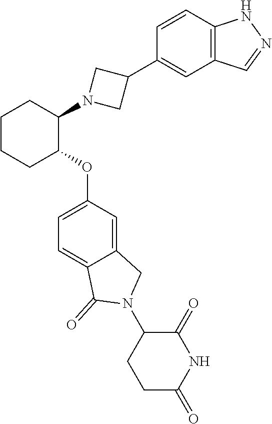 [SMILES: O=C1CCC(N2Cc3cc(O[C@@H]4CCCC[C@H]4N4CC(c5ccc6[nH]ncc6c5)C4)ccc3C2=O)C(=O)N1] 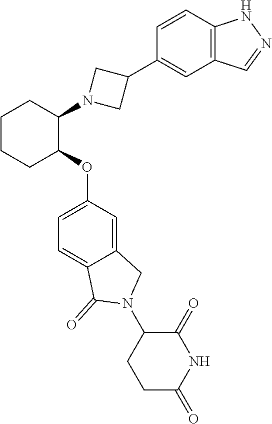 [SMILES: O=C1CCC(N2Cc3cc(O[C@H]4CCCC[C@H]4N4CC(c5ccc6[nH]ncc6c5)C4)ccc3C2=O)C(=O)N1] 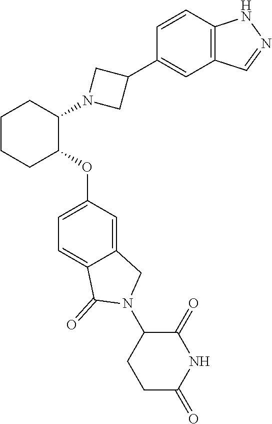 [SMILES: O=C1CCC(N2Cc3cc(O[C@@H]4CCCC[C@@H]4N4CC(c5ccc6[nH]ncc6c5)C4)ccc3C2=O)C(=O)N1] 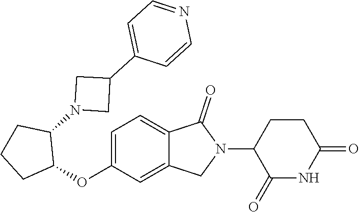 [SMILES: O=C1CCC(N2Cc3cc(O[C@@H]4CCC[C@@H]4N4CC(c5ccncc5)C4)ccc3C2=O)C(=O)N1] 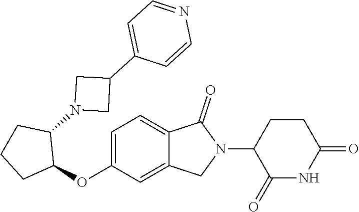 [SMILES: O=C1CCC(N2Cc3cc(O[C@H]4CCC[C@@H]4N4CC(c5ccncc5)C4)ccc3C2=O)C(=O)N1] 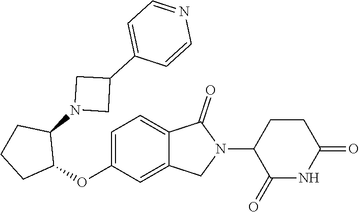 [SMILES: O=C1CCC(N2Cc3cc(O[C@@H]4CCC[C@H]4N4CC(c5ccncc5)C4)ccc3C2=O)C(=O)N1] 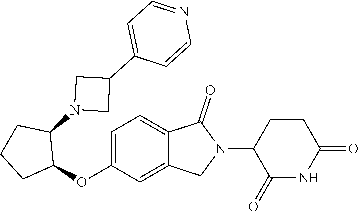 [SMILES: O=C1CCC(N2Cc3cc(O[C@H]4CCC[C@H]4N4CC(c5ccncc5)C4)ccc3C2=O)C(=O)N1] 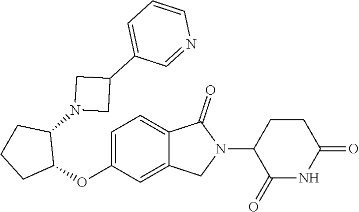 [SMILES: O=C1CCC(N2Cc3cc(O[C@@H]4CCC[C@@H]4N4CC(c5cccnc5)C4)ccc3C2=O)C(=O)N1] 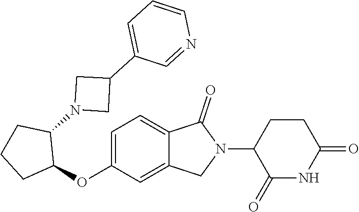 [SMILES: O=C1CCC(N2Cc3cc(O[C@H]4CCC[C@@H]4N4CC(c5cccnc5)C4)ccc3C2=O)C(=O)N1] 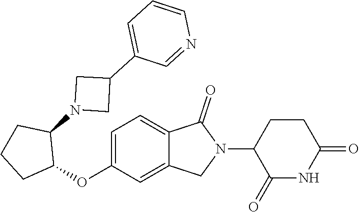 [SMILES: O=C1CCC(N2Cc3cc(O[C@@H]4CCC[C@H]4N4CC(c5cccnc5)C4)ccc3C2=O)C(=O)N1] 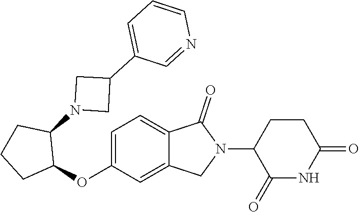 [SMILES: O=C1CCC(N2Cc3cc(O[C@H]4CCC[C@H]4N4CC(c5cccnc5)C4)ccc3C2=O)C(=O)N1] 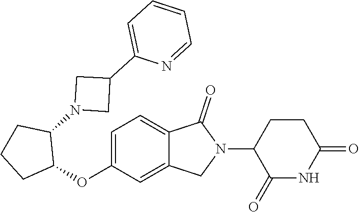 [SMILES: O=C1CCC(N2Cc3cc(O[C@@H]4CCC[C@@H]4N4CC(c5ccccn5)C4)ccc3C2=O)C(=O)N1] 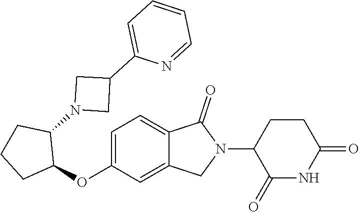 [SMILES: O=C1CCC(N2Cc3cc(O[C@H]4CCC[C@@H]4N4CC(c5ccccn5)C4)ccc3C2=O)C(=O)N1] 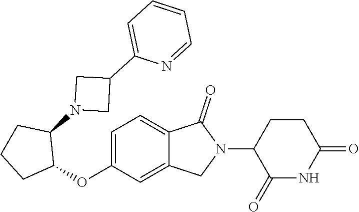 [SMILES: O=C1CCC(N2Cc3cc(O[C@@H]4CCC[C@H]4N4CC(c5ccccn5)C4)ccc3C2=O)C(=O)N1] 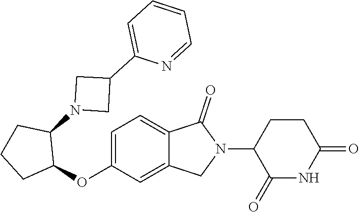 [SMILES: O=C1CCC(N2Cc3cc(O[C@H]4CCC[C@H]4N4CC(c5ccccn5)C4)ccc3C2=O)C(=O)N1] 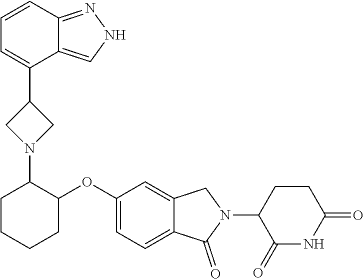 [SMILES: O=C1CCC(N2Cc3cc(OC4CCCCC4N4CC(c5cccc6n[nH]cc56)C4)ccc3C2=O)C(=O)N1] 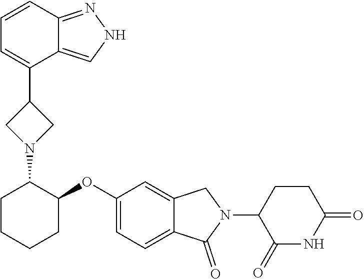 [SMILES: O=C1CCC(N2Cc3cc(O[C@H]4CCCC[C@@H]4N4CC(c5cccc6n[nH]cc56)C4)ccc3C2=O)C(=O)N1] 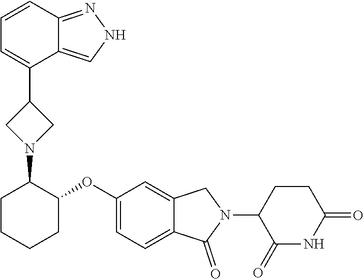 [SMILES: O=C1CCC(N2Cc3cc(O[C@@H]4CCCC[C@H]4N4CC(c5cccc6n[nH]cc56)C4)ccc3C2=O)C(=O)N1] 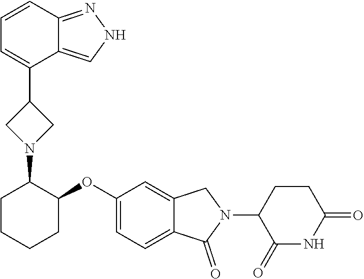 [SMILES: O=C1CCC(N2Cc3cc(O[C@H]4CCCC[C@H]4N4CC(c5cccc6n[nH]cc56)C4)ccc3C2=O)C(=O)N1] 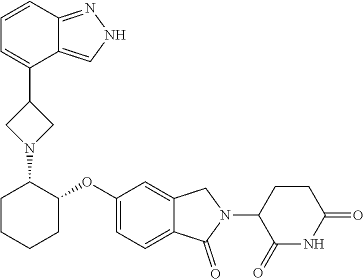 [SMILES: O=C1CCC(N2Cc3cc(O[C@@H]4CCCC[C@@H]4N4CC(c5cccc6n[nH]cc56)C4)ccc3C2=O)C(=O)N1] 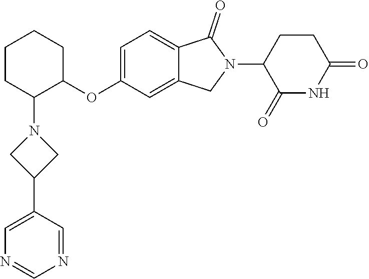 [SMILES: O=C1CCC(N2Cc3cc(OC4CCCCC4N4CC(c5cncnc5)C4)ccc3C2=O)C(=O)N1] 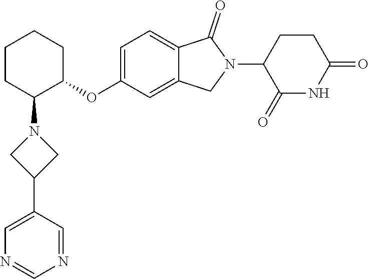 [SMILES: O=C1CCC(N2Cc3cc(O[C@H]4CCCC[C@@H]4N4CC(c5cncnc5)C4)ccc3C2=O)C(=O)N1] 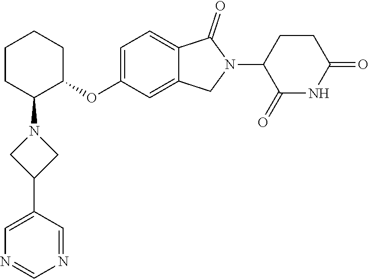 [SMILES: O=C1CCC(N2Cc3cc(O[C@H]4CCCC[C@@H]4N4CC(c5cncnc5)C4)ccc3C2=O)C(=O)N1] 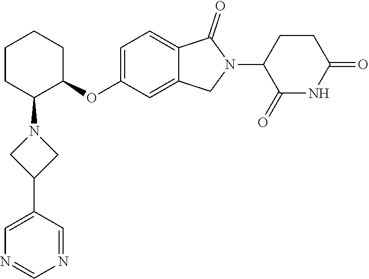 [SMILES: O=C1CCC(N2Cc3cc(O[C@@H]4CCCC[C@@H]4N4CC(c5cncnc5)C4)ccc3C2=O)C(=O)N1] 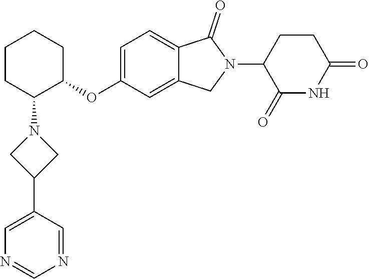 [SMILES: O=C1CCC(N2Cc3cc(O[C@H]4CCCC[C@H]4N4CC(c5cncnc5)C4)ccc3C2=O)C(=O)N1]
or wherein the compound is selected from:

| # | Structure |
|---|---|
| 1-A | 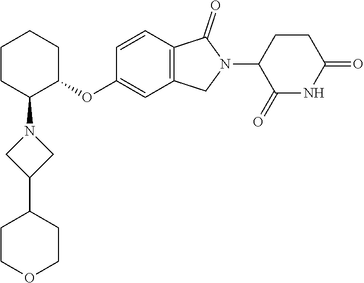 [SMILES: O=C1CCC(N2Cc3cc(O[C@H]4CCCC[C@@H]4N4CC(C5CCOCC5)C4)ccc3C2=O)C(=O)N1] |
| 12-A | 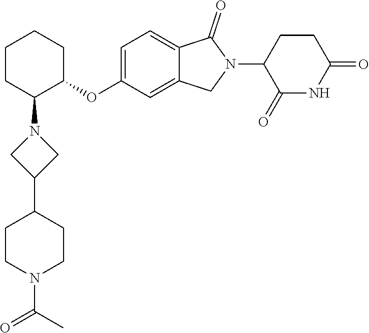 [SMILES: CC(=O)N1CCC(C2CN([C@H]3CCCC[C@@H]3Oc3ccc4c(c3)CN(C3CCC(=O)NC3=O)C4=O)C2)CC1] |
| 14-A | 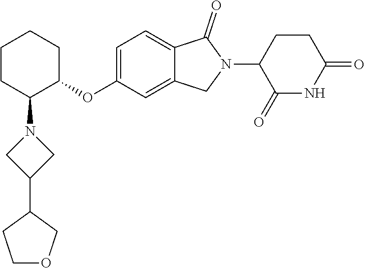 [SMILES: O=C1CCC(N2Cc3cc(O[C@H]4CCCC[C@@H]4N4CC(C5CCOC5)C4)ccc3C2=O)C(=O)N1] |
| 22-A | 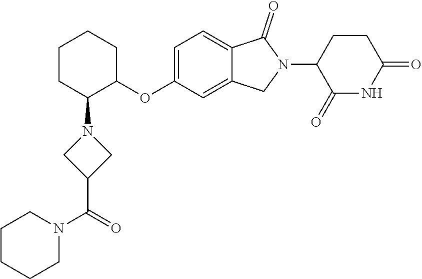 [SMILES: O=C1CCC(N2Cc3cc(OC4CCCC[C@@H]4N4CC(C(=O)N5CCCCC5)C4)ccc3C2=O)C(=O)N1] |
| | Second Eluting Isomer |
| 24-A | 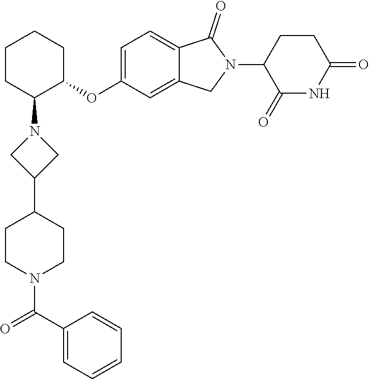 [SMILES: O=C1CCC(N2Cc3cc(O[C@H]4CCCC[C@@H]4N4CC(C5CCN(C(=O)c6ccccc6)CC5)C4)ccc3C2=O)C(=O)N1] |
| 25-A | 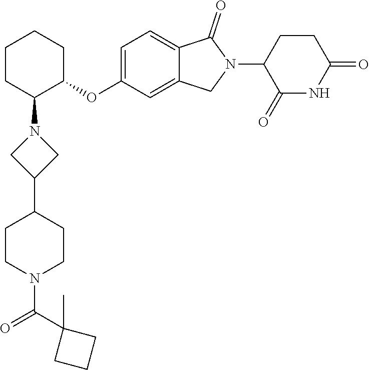 [SMILES: CC1(C(=O)N2CCC(C3CN([C@H]4CCCC[C@@H]4Oc4ccc5c(c4)CN(C4CCC(=O)NC4=O)C5=O)C3)CC2)CCC1] |
| 26-A | 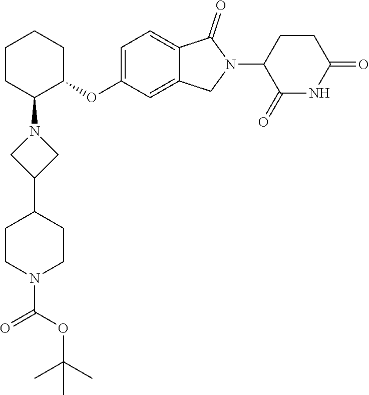 [SMILES: CC(C)(C)OC(=O)N1CCC(C2CN([C@H]3CCCC[C@@H]3Oc3ccc4c(c3)CN(C3CCC(=O)NC3=O)C4=O)C2)CC1] |
| 27-A | 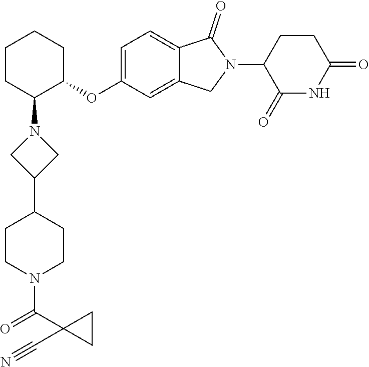 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1] |
| 28-A | 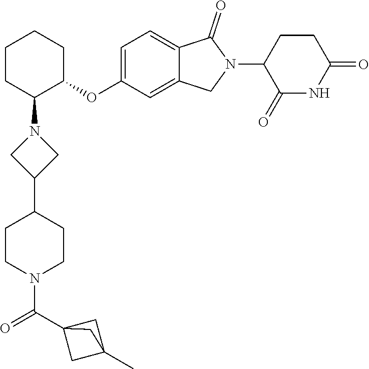 [SMILES: CC12CC(C(=O)N3CCC(C4CN([C@H]5CCCC[C@@H]5Oc5ccc6c(c5)CN(C5CCC(=O)NC5=O)C6=O)C4)CC3)(C1)C2] |
| 29-A | 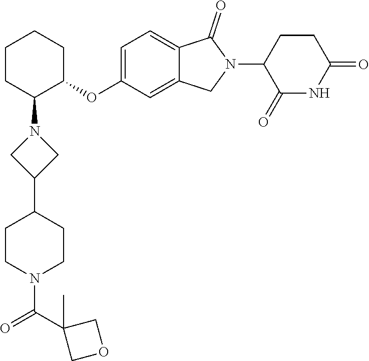 [SMILES: CC1(C(=O)N2CCC(C3CN([C@H]4CCCC[C@@H]4Oc4ccc5c(c4)CN(C4CCC(=O)NC4=O)C5=O)C3)CC2)COC1] |
| 30-A | 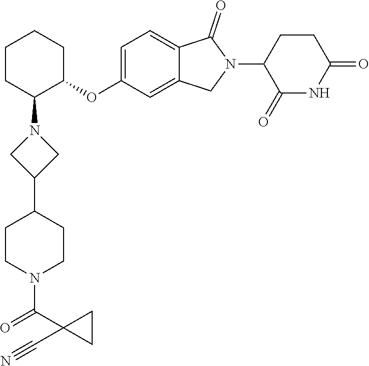 [SMILES: N#CC1(C(=O)N2CCC(C3CN([C@H]4CCCC[C@@H]4Oc4ccc5c(c4)CN(C4CCC(=O)NC4=O)C5=O)C3)CC2)CC1] |
| 31-A | 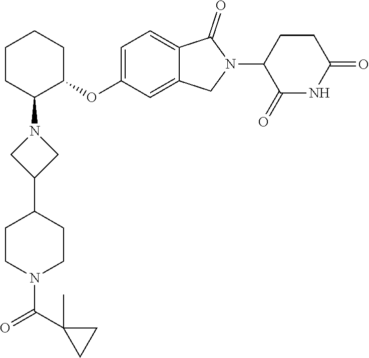 [SMILES: CC1(C(=O)N2CCC(C3CN([C@H]4CCCC[C@@H]4Oc4ccc5c(c4)CN(C4CCC(=O)NC4=O)C5=O)C3)CC2)CC1] |
| 32-A | 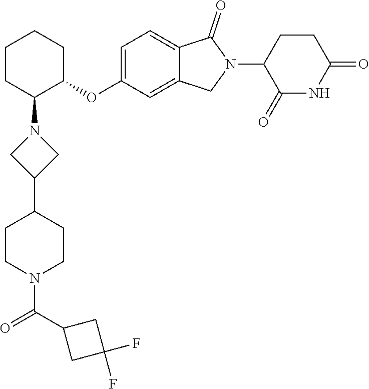 [SMILES: O=C1CCC(N2Cc3cc(O[C@H]4CCCC[C@@H]4N4CC(C5CCN(C(=O)C6CC(F)(F)C6)CC5)C4)ccc3C2=O)C(=O)N1] |
| 33-A | 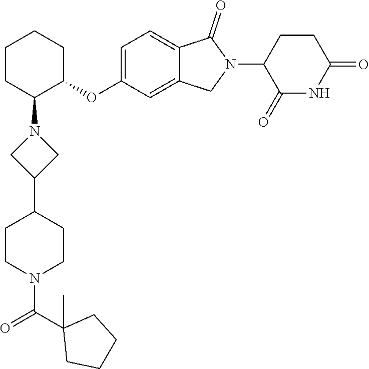 [SMILES: CC1(C(=O)N2CCC(C3CN([C@H]4CCCC[C@@H]4Oc4ccc5c(c4)CN(C4CCC(=O)NC4=O)C5=O)C3)CC2)CCCC1] |
| 34-A | 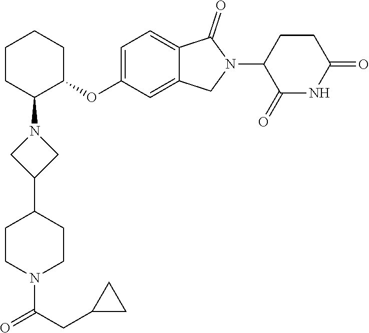 [SMILES: O=C1CCC(N2Cc3cc(O[C@H]4CCCC[C@@H]4N4CC(C5CCN(C(=O)CC6CC6)CC5)C4)ccc3C2=O)C(=O)N1] |
| 35-A | 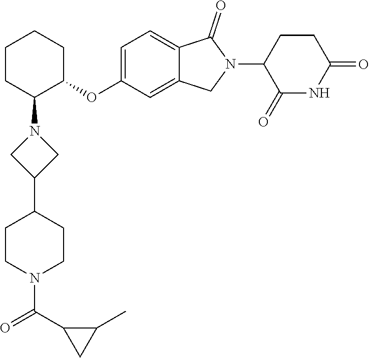 [SMILES: CC1CC1C(=O)N1CCC(C2CN([C@H]3CCCC[C@@H]3Oc3ccc4c(c3)CN(C3CCC(=O)NC3=O)C4=O)C2)CC1] |
| 41-A | 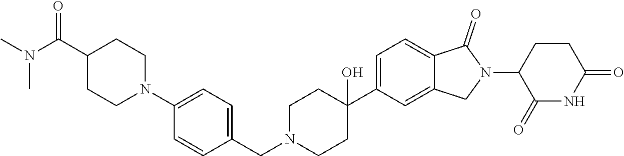 [SMILES: CN(C)C(=O)C1CCN(c2ccc(CN3CCC(O)(c4ccc5c(c4)CN(C4CCC(=O)NC4=O)C5=O)CC3)cc2)CC1] |
| 42-A | 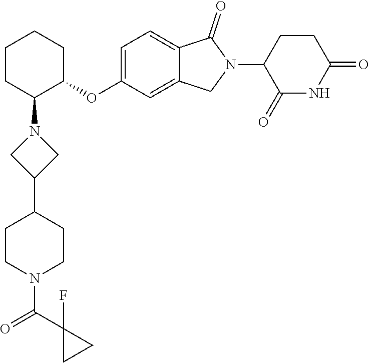 [SMILES: O=C1CCC(N2Cc3cc(O[C@H]4CCCC[C@@H]4N4CC(C5CCN(C(=O)C6(F)CC6)CC5)C4)ccc3C2=O)C(=O)N1] |
| 55-A | 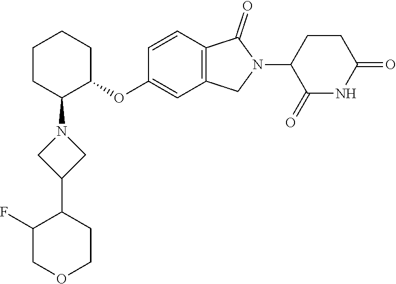 [SMILES: O=C1CCC(N2Cc3cc(O[C@H]4CCCC[C@@H]4N4CC(C5CCOCC5F)C4)ccc3C2=O)C(=O)N1] |
| 56-A | 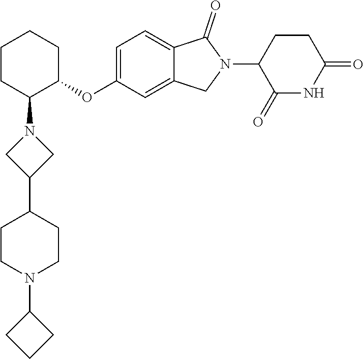 [SMILES: O=C1CCC(N2Cc3cc(O[C@H]4CCCC[C@@H]4N4CC(C5CCN(C6CCC6)CC5)C4)ccc3C2=O)C(=O)N1] |
| 57-A | 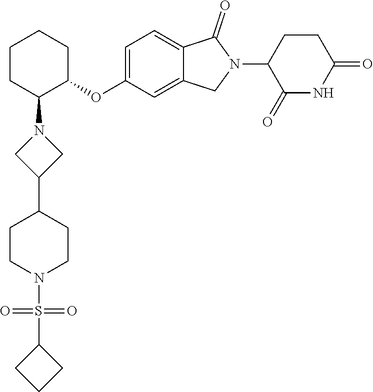 [SMILES: O=C1CCC(N2Cc3cc(O[C@H]4CCCC[C@@H]4N4CC(C5CCN(S(=O)(=O)C6CCC6)CC5)C4)ccc3C2=O)C(=O)N1] |
| 58-A | 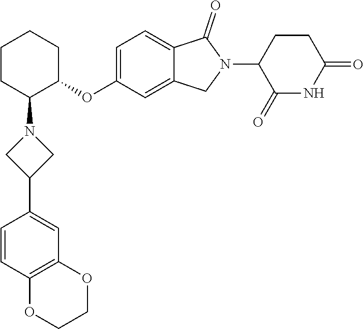 [SMILES: O=C1CCC(N2Cc3cc(O[C@H]4CCCC[C@@H]4N4CC(c5ccc6c(c5)OCCO6)C4)ccc3C2=O)C(=O)N1] |
| 1-B | 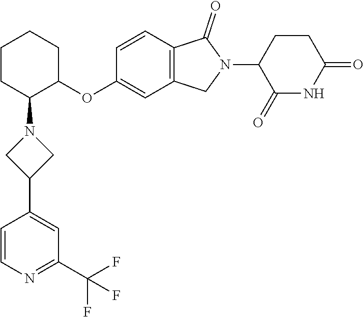 [SMILES: O=C1CCC(N2Cc3cc(OC4CCCC[C@@H]4N4CC(c5ccnc(C(F)(F)F)c5)C4)ccc3C2=O)C(=O)N1] |
| 2-B | 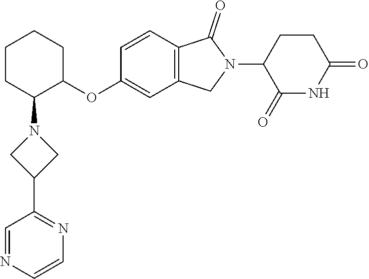 [SMILES: O=C1CCC(N2Cc3cc(OC4CCCC[C@@H]4N4CC(c5cnccn5)C4)ccc3C2=O)C(=O)N1] |
| 6-B | 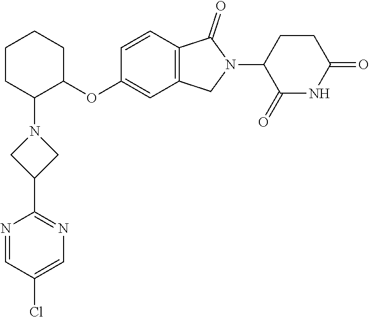 [SMILES: O=C1CCC(N2Cc3cc(OC4CCCCC4N4CC(c5ncc(Cl)cn5)C4)ccc3C2=O)C(=O)N1] |
| 11-B | 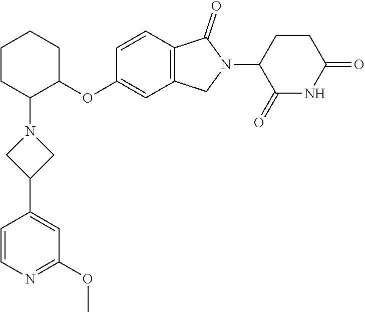 [SMILES: COc1cc(C2CN(C3CCCCC3Oc3ccc4c(c3)CN(C3CCC(=O)NC3=O)C4=O)C2)ccn1] |
| 16-B | 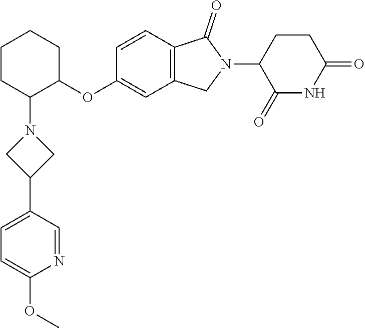 [SMILES: COc1ccc(C2CN(C3CCCCC3Oc3ccc4c(c3)CN(C3CCC(=O)NC3=O)C4=O)C2)cn1] |
| 21-B | 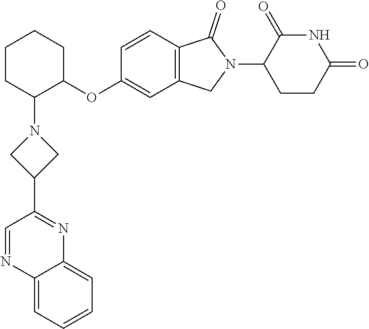 [SMILES: O=C1CCC(N2Cc3cc(OC4CCCCC4N4CC(c5cnc6ccccc6n5)C4)ccc3C2=O)C(=O)N1] |
| 26-B | 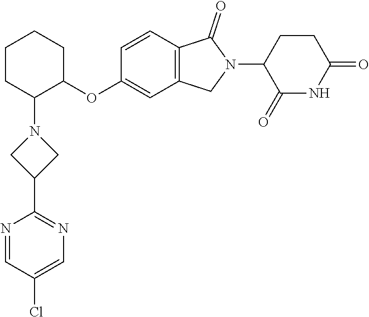 [SMILES: O=C1CCC(N2Cc3cc(OC4CCCCC4N4CC(c5ncc(Cl)cn5)C4)ccc3C2=O)C(=O)N1] |
| 31-B | 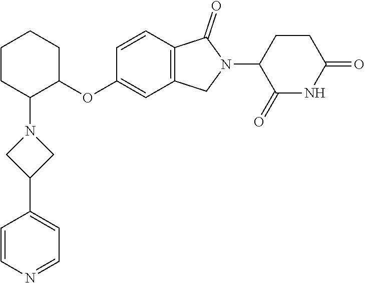 [SMILES: O=C1CCC(N2Cc3cc(OC4CCCCC4N4CC(c5ccncc5)C4)ccc3C2=O)C(=O)N1] |
| 36-B | 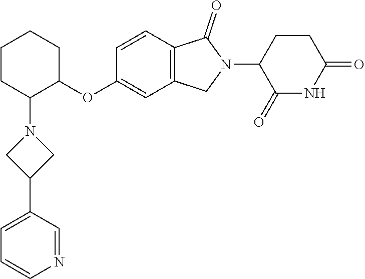 [SMILES: O=C1CCC(N2Cc3cc(OC4CCCCC4N4CC(c5cccnc5)C4)ccc3C2=O)C(=O)N1] |
| 46-B | 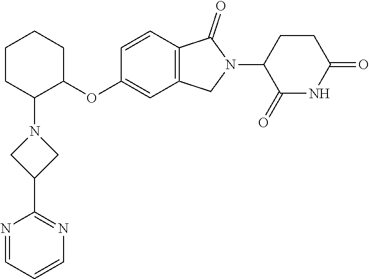 [SMILES: O=C1CCC(N2Cc3cc(OC4CCCCC4N4CC(c5ncccn5)C4)ccc3C2=O)C(=O)N1] |
| 56-B | 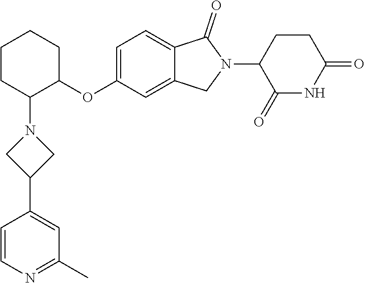 [SMILES: Cc1cc(C2CN(C3CCCCC3Oc3ccc4c(c3)CN(C3CCC(=O)NC3=O)C4=O)C2)ccn1] |
| 61-B | 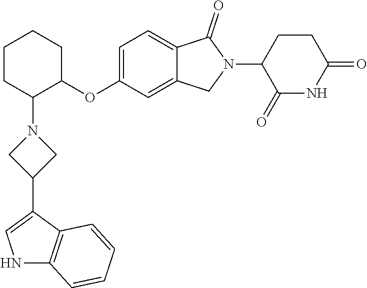 [SMILES: O=C1CCC(N2Cc3cc(OC4CCCCC4N4CC(c5c[nH]c6ccccc56)C4)ccc3C2=O)C(=O)N1] |
| 66-B | 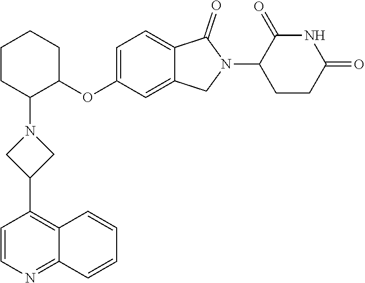 [SMILES: O=C1CCC(N2Cc3cc(OC4CCCCC4N4CC(c5ccnc6ccccc56)C4)ccc3C2=O)C(=O)N1] |
| 71-B | 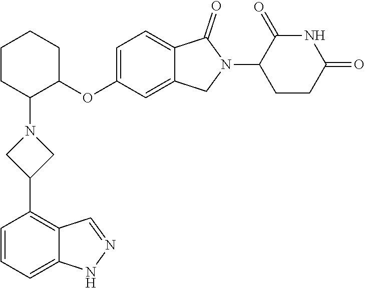 [SMILES: O=C1CCC(N2Cc3cc(OC4CCCCC4N4CC(c5cccc6[nH]ncc56)C4)ccc3C2=O)C(=O)N1] |
| 76-B | 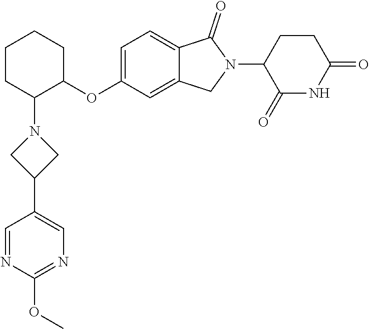 [SMILES: COc1ncc(C2CN(C3CCCCC3Oc3ccc4c(c3)CN(C3CCC(=O)NC3=O)C4=O)C2)cn1] |
| 81-B | 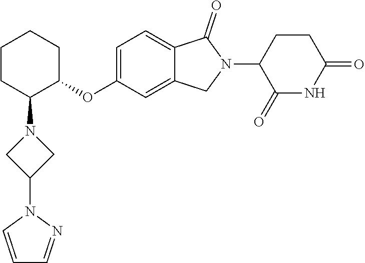 [SMILES: O=C1CCC(N2Cc3cc(O[C@H]4CCCC[C@@H]4N4CC(n5cccn5)C4)ccc3C2=O)C(=O)N1] |
| 87-B | 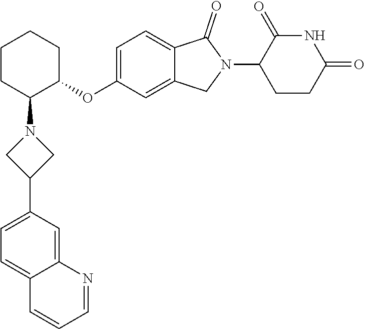 [SMILES: O=C1CCC(N2Cc3cc(O[C@H]4CCCC[C@@H]4N4CC(c5ccc6cccnc6c5)C4)ccc3C2=O)C(=O)N1] |
| 89-B | 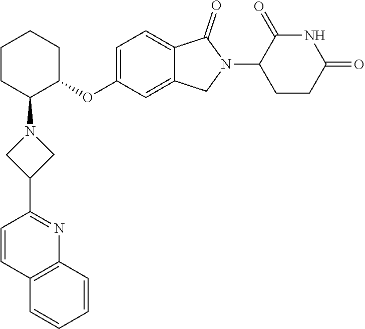 [SMILES: O=C1CCC(N2Cc3cc(O[C@H]4CCCC[C@@H]4N4CC(c5ccc6ccccc6n5)C4)ccc3C2=O)C(=O)N1] |
| | Diastereomer 1 |
| 90-B | 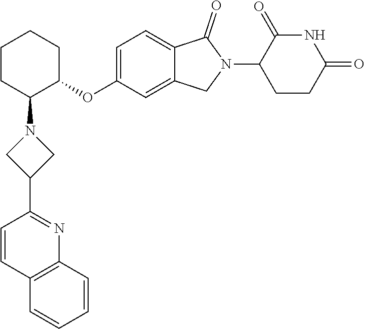 [SMILES: O=C1CCC(N2Cc3cc(O[C@H]4CCCC[C@@H]4N4CC(c5ccc6ccccc6n5)C4)ccc3C2=O)C(=O)N1] |
| | Diastereomer 2 |
| 91-B | 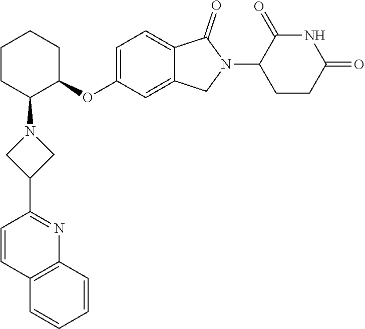 [SMILES: O=C1CCC(N2Cc3cc(O[C@@H]4CCCC[C@@H]4N4CC(c5ccc6ccccc6n5)C4)ccc3C2=O)C(=O)N1] |
| | Diastereomer 3 |
| 92-B | 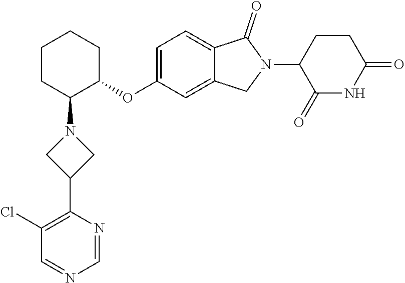 [SMILES: O=C1CCC(N2Cc3cc(O[C@H]4CCCC[C@@H]4N4CC(c5ncncc5Cl)C4)ccc3C2=O)C(=O)N1] |
| | Diastereomer 1 |
| 93-B | 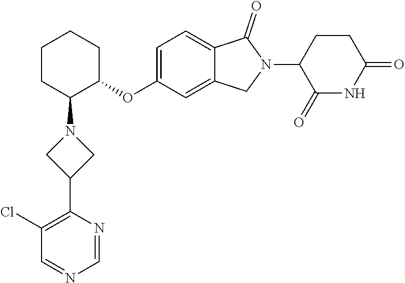 [SMILES: O=C1CCC(N2Cc3cc(O[C@H]4CCCC[C@@H]4N4CC(c5ncncc5Cl)C4)ccc3C2=O)C(=O)N1] |
| | Diastereomer 2 |
| 95-B | 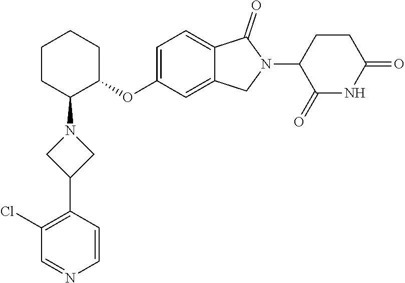 [SMILES: O=C1CCC(N2Cc3cc(O[C@H]4CCCC[C@@H]4N4CC(c5ccncc5Cl)C4)ccc3C2=O)C(=O)N1] |
| | Diastereomer 1 |
| 98-B | 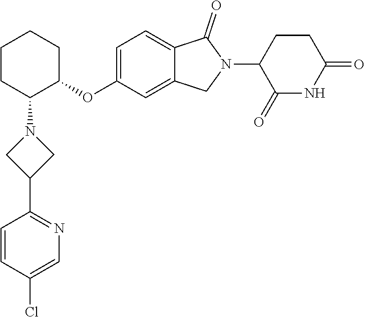 [SMILES: O=C1CCC(N2Cc3cc(O[C@H]4CCCC[C@H]4N4CC(c5ccc(Cl)cn5)C4)ccc3C2=O)C(=O)N1] |
| | Diastereomer 2 |
| 99-B | 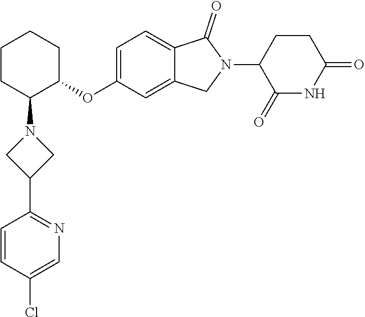 [SMILES: O=C1CCC(N2Cc3cc(O[C@H]4CCCC[C@@H]4N4CC(c5ccc(Cl)cn5)C4)ccc3C2=O)C(=O)N1] |
| | Diastereomer 3 |
| 100-B | 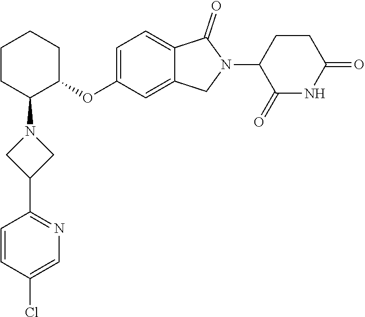 [SMILES: O=C1CCC(N2Cc3cc(O[C@H]4CCCC[C@@H]4N4CC(c5ccc(Cl)cn5)C4)ccc3C2=O)C(=O)N1] |
| | Diastereomer 4 |
| 102-B | 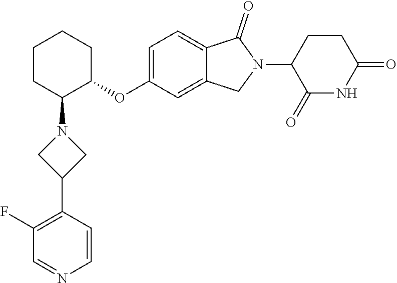 [SMILES: O=C1CCC(N2Cc3cc(O[C@H]4CCCC[C@@H]4N4CC(c5ccncc5F)C4)ccc3C2=O)C(=O)N1] |
| | Diastereomer 1 |
| 103-B | 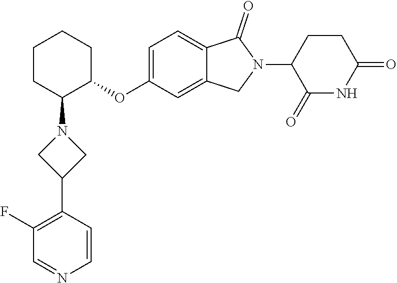 [SMILES: O=C1CCC(N2Cc3cc(O[C@H]4CCCC[C@@H]4N4CC(c5ccncc5F)C4)ccc3C2=O)C(=O)N1] |
| | Diastereomer 2 |
| 108-B | 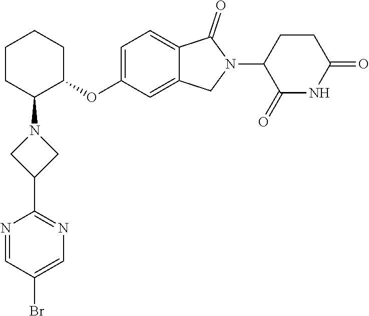 [SMILES: O=C1CCC(N2Cc3cc(O[C@H]4CCCC[C@@H]4N4CC(c5ncc(Br)cn5)C4)ccc3C2=O)C(=O)N1] |
| | Diastereomer 3 |
| 111-B | 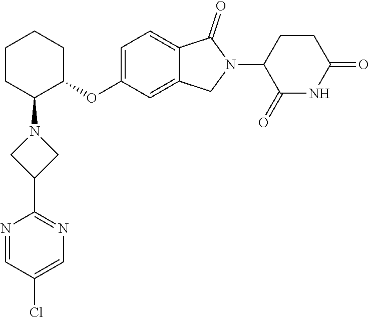 [SMILES: O=C1CCC(N2Cc3cc(O[C@H]4CCCC[C@@H]4N4CC(c5ncc(Cl)cn5)C4)ccc3C2=O)C(=O)N1] |
| | Diastereomer 3 |
| 112-B | 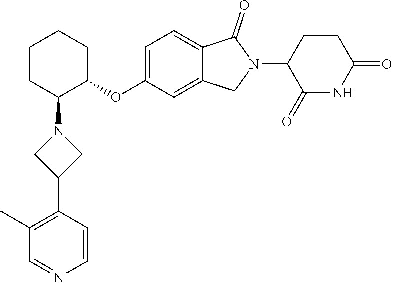 [SMILES: Cc1cnccc1C1CN([C@H]2CCCC[C@@H]2Oc2ccc3c(c2)CN(C2CCC(=O)NC2=O)C3=O)C1] |
| | Diastereomer 1 |
| 116-B | 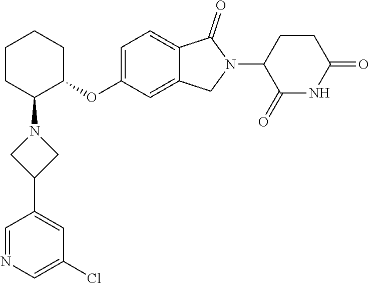 [SMILES: O=C1CCC(N2Cc3cc(O[C@H]4CCCC[C@@H]4N4CC(c5cncc(Cl)c5)C4)ccc3C2=O)C(=O)N1] |
| | Diastereomer 4 |
| 117-B | 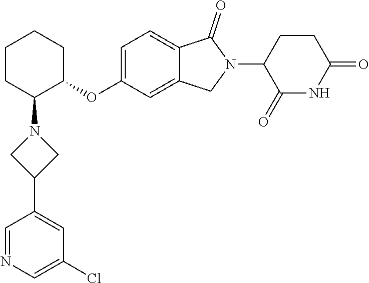 [SMILES: O=C1CCC(N2Cc3cc(O[C@H]4CCCC[C@@H]4N4CC(c5cncc(Cl)c5)C4)ccc3C2=O)C(=O)N1] |
| | Diastereomer 5 |
| 120-B | 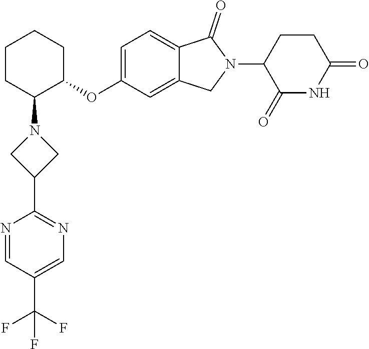 [SMILES: O=C1CCC(N2Cc3cc(O[C@H]4CCCC[C@@H]4N4CC(c5ncc(C(F)(F)F)cn5)C4)ccc3C2=O)C(=O)N1] |
| | Diastereomer 3 |
| 123-B | 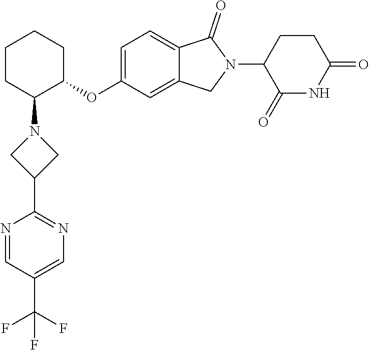 [SMILES: O=C1CCC(N2Cc3cc(O[C@H]4CCCC[C@@H]4N4CC(c5ncc(C(F)(F)F)cn5)C4)ccc3C2=O)C(=O)N1] |
| | Diastereomer 6 |
| 125-B | 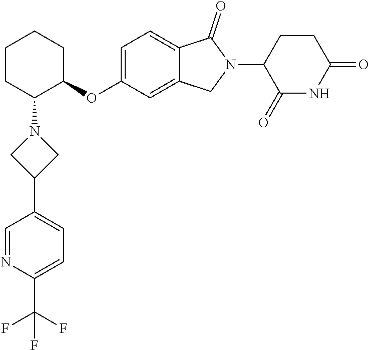 [SMILES: O=C1CCC(N2Cc3cc(O[C@@H]4CCCC[C@H]4N4CC(c5ccc(C(F)(F)F)nc5)C4)ccc3C2=O)C(=O)N1] |
| | Diastereomer 2 |
| 126-B | 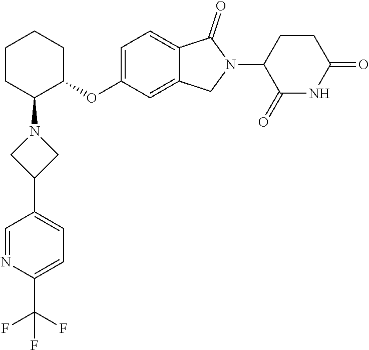 [SMILES: O=C1CCC(N2Cc3cc(O[C@H]4CCCC[C@@H]4N4CC(c5ccc(C(F)(F)F)nc5)C4)ccc3C2=O)C(=O)N1] |
| | Diastereomer 3 |
| 127-B | 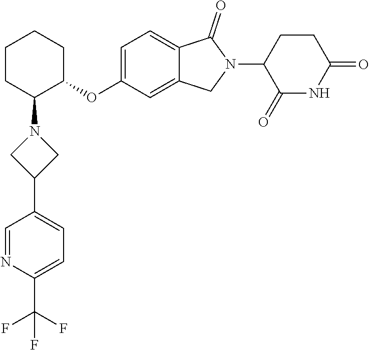 [SMILES: O=C1CCC(N2Cc3cc(O[C@H]4CCCC[C@@H]4N4CC(c5ccc(C(F)(F)F)nc5)C4)ccc3C2=O)C(=O)N1] |
| | Diastereomer 4 |
| 129-B | 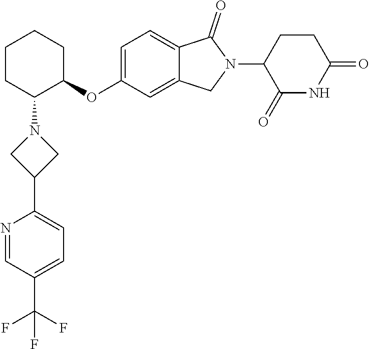 [SMILES: O=C1CCC(N2Cc3cc(O[C@@H]4CCCC[C@H]4N4CC(c5ccc(C(F)(F)F)cn5)C4)ccc3C2=O)C(=O)N1] |
| | Diastereomer 2 |
| 130-B | 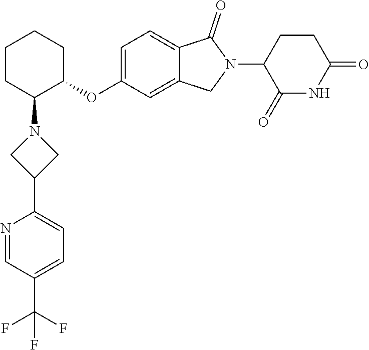 [SMILES: O=C1CCC(N2Cc3cc(O[C@H]4CCCC[C@@H]4N4CC(c5ccc(C(F)(F)F)cn5)C4)ccc3C2=O)C(=O)N1] |
| | Diastereomer 3 |
| 131-B | 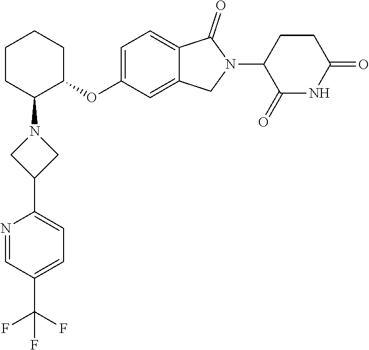 [SMILES: O=C1CCC(N2Cc3cc(O[C@H]4CCCC[C@@H]4N4CC(c5ccc(C(F)(F)F)cn5)C4)ccc3C2=O)C(=O)N1] |
| | Diastereomer 4 |
| 134-B | 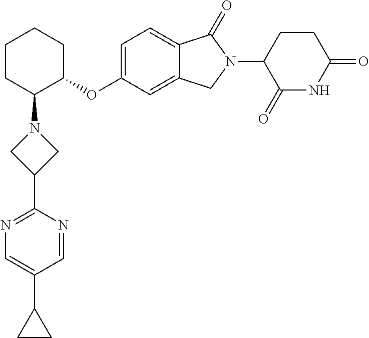 [SMILES: O=C1CCC(N2Cc3cc(O[C@H]4CCCC[C@@H]4N4CC(c5ncc(C6CC6)cn5)C4)ccc3C2=O)C(=O)N1] |
| | Diastereomer 3 |
| 135-B | 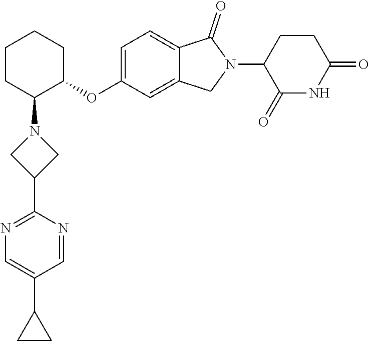 [SMILES: O=C1CCC(N2Cc3cc(O[C@H]4CCCC[C@@H]4N4CC(c5ncc(C6CC6)cn5)C4)ccc3C2=O)C(=O)N1] |
| | Diastereomer 4 |
| 136-B | 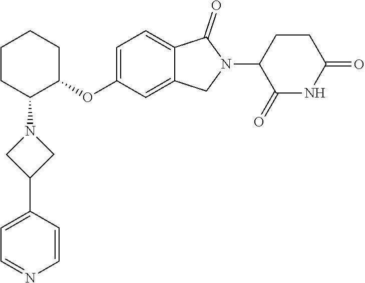 [SMILES: O=C1CCC(N2Cc3cc(O[C@H]4CCCC[C@H]4N4CC(c5ccncc5)C4)ccc3C2=O)C(=O)N1] |
| | Diastereomer 3 |
| 137-B | 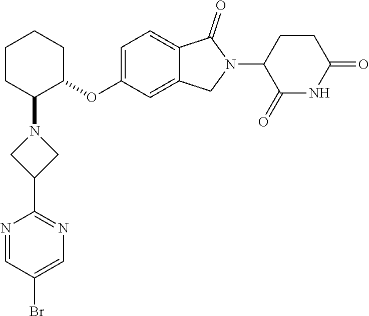 [SMILES: O=C1CCC(N2Cc3cc(O[C@H]4CCCC[C@@H]4N4CC(c5ncc(Br)cn5)C4)ccc3C2=O)C(=O)N1] |
| 139-B | 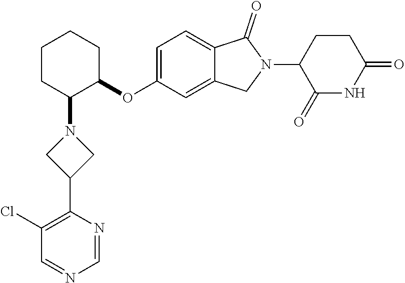 [SMILES: O=C1CCC(N2Cc3cc(O[C@@H]4CCCC[C@@H]4N4CC(c5ncncc5Cl)C4)ccc3C2=O)C(=O)N1] |
| 142-B | 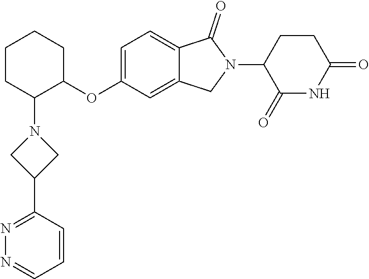 [SMILES: O=C1CCC(N2Cc3cc(OC4CCCCC4N4CC(c5cccnn5)C4)ccc3C2=O)C(=O)N1] |
| 146-B | 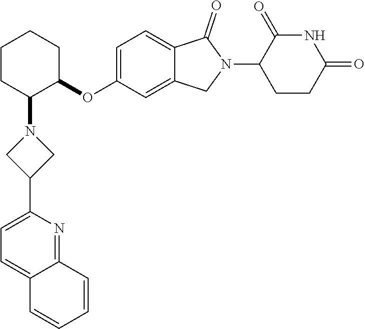 [SMILES: O=C1CCC(N2Cc3cc(O[C@@H]4CCCC[C@@H]4N4CC(c5ccc6ccccc6n5)C4)ccc3C2=O)C(=O)N1] |
| 152-B | 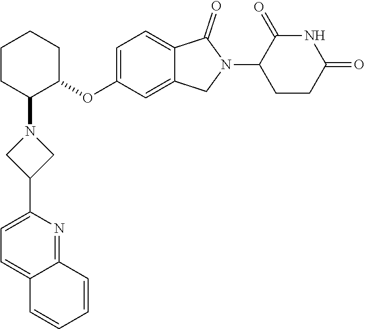 [SMILES: O=C1CCC(N2Cc3cc(O[C@H]4CCCC[C@@H]4N4CC(c5ccc6ccccc6n5)C4)ccc3C2=O)C(=O)N1] |
| 153-B | 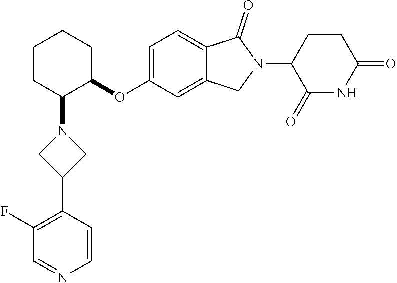 [SMILES: O=C1CCC(N2Cc3cc(O[C@@H]4CCCC[C@@H]4N4CC(c5ccncc5F)C4)ccc3C2=O)C(=O)N1] |
| 155-B | 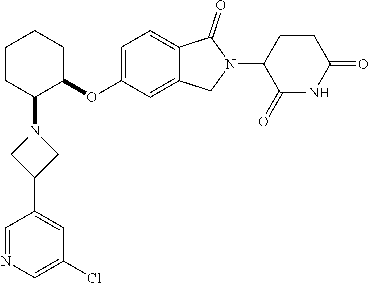 [SMILES: O=C1CCC(N2Cc3cc(O[C@@H]4CCCC[C@@H]4N4CC(c5cncc(Cl)c5)C4)ccc3C2=O)C(=O)N1] |
| 161-B | 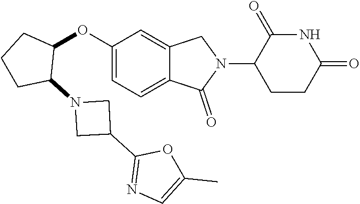 |
| 162-B | 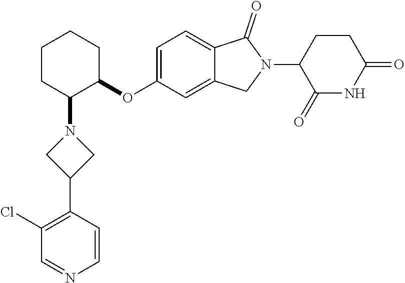 [SMILES: O=C1CCC(N2Cc3cc(O[C@@H]4CCCC[C@@H]4N4CC(c5ccncc5Cl)C4)ccc3C2=O)C(=O)N1] |
| 163-B | 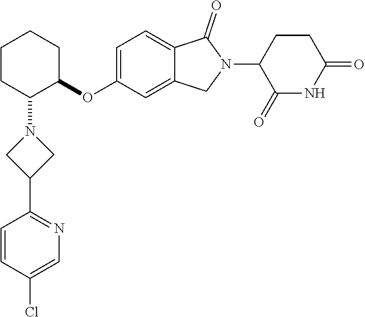 [SMILES: O=C1CCC(N2Cc3cc(O[C@@H]4CCCC[C@H]4N4CC(c5ccc(Cl)cn5)C4)ccc3C2=O)C(=O)N1] |
| 165-B | 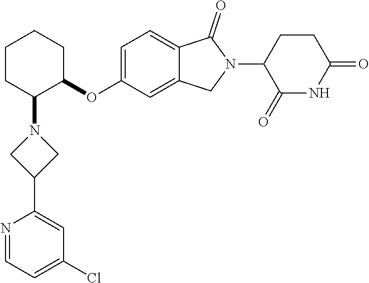 [SMILES: O=C1CCC(N2Cc3cc(O[C@@H]4CCCC[C@@H]4N4CC(c5cc(Cl)ccn5)C4)ccc3C2=O)C(=O)N1] |
| 169-B | 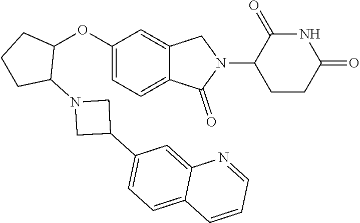 [SMILES: O=C1CCC(N2Cc3cc(OC4CCCC4N4CC(c5ccc6cccnc6c5)C4)ccc3C2=O)C(=O)N1] |
| 170-B | 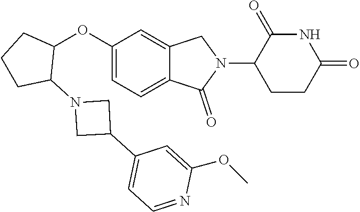 [SMILES: COc1cc(C2CN(C3CCCC3Oc3ccc4c(c3)CN(C3CCC(=O)NC3=O)C4=O)C2)ccn1] |
| 173-B | 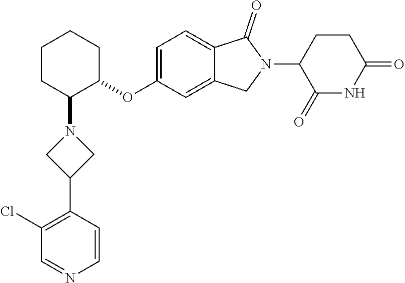 [SMILES: O=C1CCC(N2Cc3cc(O[C@H]4CCCC[C@@H]4N4CC(c5ccncc5Cl)C4)ccc3C2=O)C(=O)N1] |
| 174-B | 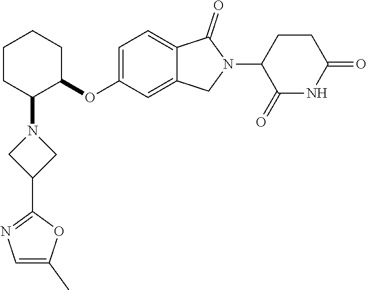 [SMILES: Cc1cnc(C2CN([C@H]3CCCC[C@H]3Oc3ccc4c(c3)CN(C3CCC(=O)NC3=O)C4=O)C2)o1] |
| 177-B | 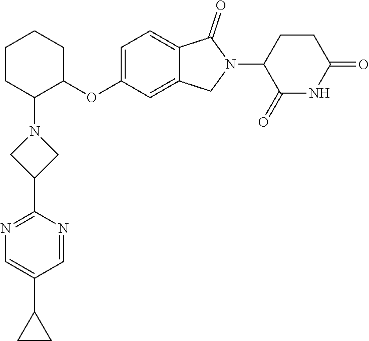 [SMILES: O=C1CCC(N2Cc3cc(OC4CCCCC4N4CC(c5ncc(C6CC6)cn5)C4)ccc3C2=O)C(=O)N1] |
| 179-B | 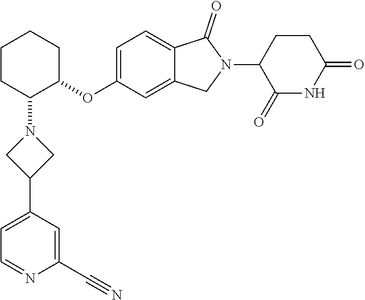 [SMILES: N#Cc1cc(C2CN([C@@H]3CCCC[C@@H]3Oc3ccc4c(c3)CN(C3CCC(=O)NC3=O)C4=O)C2)ccn1] |
| 182-B | 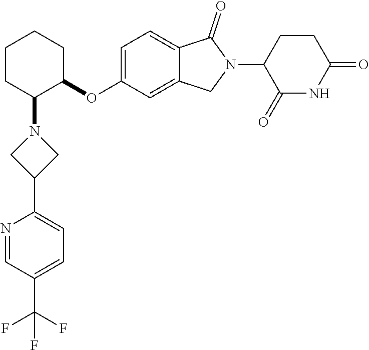 [SMILES: O=C1CCC(N2Cc3cc(O[C@@H]4CCCC[C@@H]4N4CC(c5ccc(C(F)(F)F)cn5)C4)ccc3C2=O)C(=O)N1] |
| 183-B | 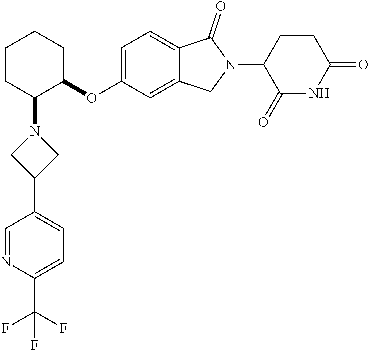 [SMILES: O=C1CCC(N2Cc3cc(O[C@@H]4CCCC[C@@H]4N4CC(c5ccc(C(F)(F)F)nc5)C4)ccc3C2=O)C(=O)N1] |
| 184-B | 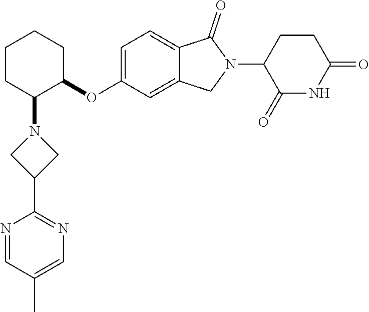 [SMILES: Cc1cnc(C2CN([C@H]3CCCC[C@H]3Oc3ccc4c(c3)CN(C3CCC(=O)NC3=O)C4=O)C2)nc1] |
| 185-B | 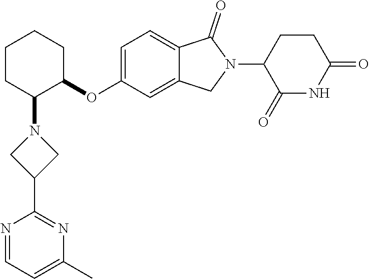 [SMILES: Cc1ccnc(C2CN([C@H]3CCCC[C@H]3Oc3ccc4c(c3)CN(C3CCC(=O)NC3=O)C4=O)C2)n1] |
| 186-B | 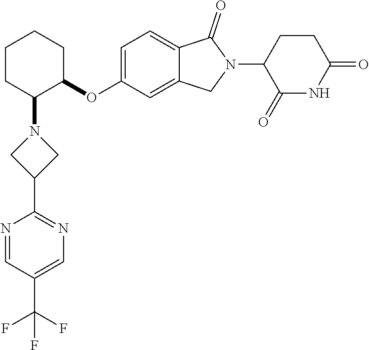 [SMILES: O=C1CCC(N2Cc3cc(O[C@@H]4CCCC[C@@H]4N4CC(c5ncc(C(F)(F)F)cn5)C4)ccc3C2=O)C(=O)N1] |
| 188-B | 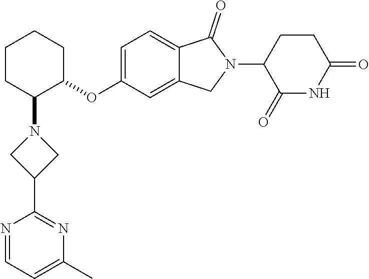 [SMILES: Cc1ccnc(C2CN([C@H]3CCCC[C@@H]3Oc3ccc4c(c3)CN(C3CCC(=O)NC3=O)C4=O)C2)n1] |
| 190-B | 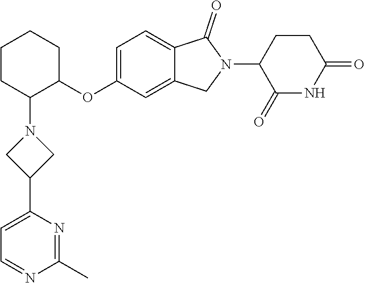 [SMILES: Cc1nccc(C2CN(C3CCCCC3Oc3ccc4c(c3)CN(C3CCC(=O)NC3=O)C4=O)C2)n1] |
| 193-B | 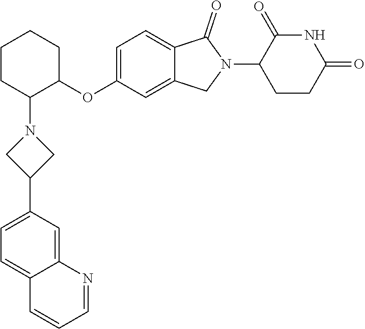 [SMILES: O=C1CCC(N2Cc3cc(OC4CCCCC4N4CC(c5ccc6cccnc6c5)C4)ccc3C2=O)C(=O)N1] |
| 194-B | 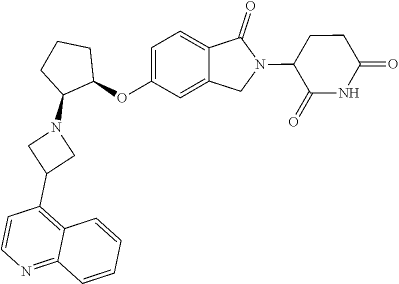 [SMILES: O=C1CCC(N2Cc3cc(O[C@@H]4CCC[C@@H]4N4CC(c5ccnc6ccccc56)C4)ccc3C2=O)C(=O)N1] |
| 198-B | 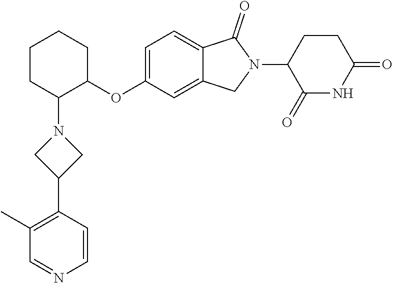 [SMILES: Cc1cnccc1C1CN(C2CCCCC2Oc2ccc3c(c2)CN(C2CCC(=O)NC2=O)C3=O)C1] |
| 199-B | 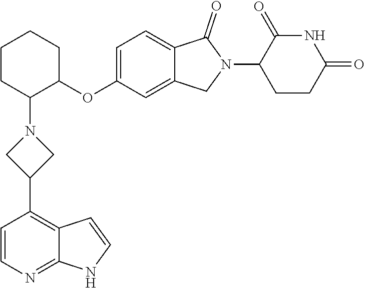 [SMILES: O=C1CCC(N2Cc3cc(OC4CCCCC4N4CC(c5ccnc6[nH]ccc56)C4)ccc3C2=O)C(=O)N1] |
| 201-B | 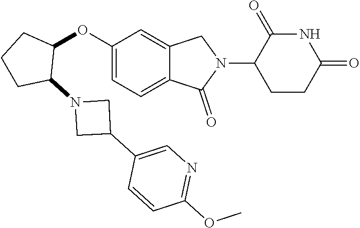 |
| 204-B | 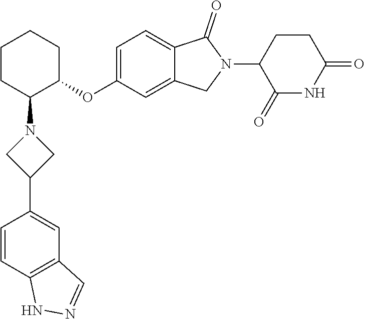 [SMILES: O=C1CCC(N2Cc3cc(O[C@H]4CCCC[C@@H]4N4CC(c5ccc6[nH]ncc6c5)C4)ccc3C2=O)C(=O)N1] |
| 205-B | 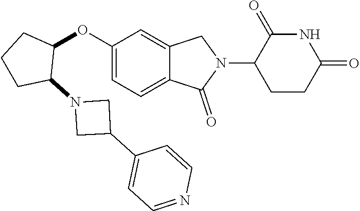 |
| 209-B | 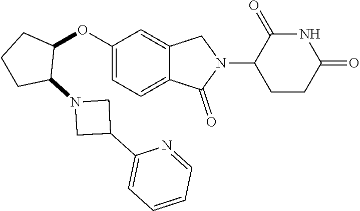 |
| 211-B | 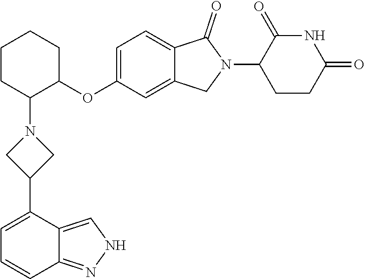 [SMILES: O=C1CCC(N2Cc3cc(OC4CCCCC4N4CC(c5cccc6n[nH]cc56)C4)ccc3C2=O)C(=O)N1] |

64.-65. (canceled)

66. A pharmaceutical composition, comprising a therapeutically effective amount of the compound or a pharmaceutically acceptable salt, solvate, stereoisomer, or tautomer thereof of claim 1, and a pharmaceutically acceptable carrier.

67. A method for modulating cereblon activity, which method comprises contacting cereblon with an effective amount of a compound of claim 1, or a pharmaceutically acceptable salt, solvate, stereoisomer, or tautomer thereof, under conditions wherein cereblon is modulated.

68. A method for degrading IKZF2, which method comprises contacting IKZF2 with an effective amount of a compound of claim 1, or a pharmaceutically acceptable salt, solvate, stereoisomer, or tautomer thereof, under conditions wherein IKZF2 is degraded.

69. A method for degrading IKZF2 in a subject, which method comprises administering to said subject an effective amount of a compound according to claim 1, or a pharmaceutically acceptable salt, solvate, stereoisomer, or tautomer thereof.

70. A method for treating cancer in a subject in need thereof, which method comprises administering to said subject an effective amount of a compound of claim 1, or a pharmaceutically acceptable salt, solvate, stereoisomer, or tautomer thereof.

71. A method for treating cancer in a subject in need thereof, which method comprises administering to said subject an effective amount of a pharmaceutical composition comprising a pharmaceutically acceptable excipient and an effective amount of a compound according to claim 1, or a pharmaceutically acceptable salt, solvate, stereoisomer, or tautomer thereof.


SEQUENCE LISTING (3 sequences)

SEQ ID NO: 1 - DNA, 3075 bp, synthetic construct
  source 1..3075: mol_type = other DNA
atggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagcggtggaggcggttcaggtggcggaggaagcgggggaggtggaagtatggtgtctaaaggagaagaagataatatggcttctctgcctgctacacacgaactgcatattttcggatctattaatggcgtggatttcgatatggtgggacagggaacaggaaaccctaacgatggatacgaagaactgaatctgaagtctaccaaaggagatctgcaattctctccttggatcctggtccctcacattggatatggattccatcaatatctgccttaccctgacggaatgtctcctttccaagctgctatggtcgatggctctggatatcaggtccacagaacaatgcaatttgaagatggcgcctctctgacagtgaattacagatatacatacgaaggctctcatattaaaggcgaagcccaagtgaaaggcacaggctttcctgccgacggccctgtgatgaccaactctctgaccgctgccgattggtgcagatctaagaagacctaccctaatgataaaaccattatctctaccttcaaatggtcttacacaacaggcaatggcaagagatacagatctaccgccagaaccacctatacctttgctaagcctatggctgccaattacctgaaaaatcagcctatgtatgtgttcagaaaaaccgaactgaaacactctaaaaccgaactgaatttcaaagaatggcaaaaggctttcacagatgtgatgggaatggatgaactgtacaagggaagcggagctacaaattttagtcttcttaaacaagccggtgacgtggaggaaaatcccggcccaatggtgagcaagggcgaggccgtgatcaaggagttcatgcgcttcaaggtgcacatggagggcagcatgaacggccacgagttcgagatcgagggcgagggcgagggccgcccctacgagggcacccagaccgccaagctgaaggtgaccaagggcggccccctgcccttctcttgggacatcctgagcccccagttcatgtacggcagccgcgccttcaccaagcaccccgccgacatccccgactactacaagcagagcttccccgagggcttcaagtgggagcgcgtgatgaacttcgaggacggcggcgccgtgaccgtgacccaggacaccagcctggaggacggcaccctgatctacaaggtgaagctgcgcggcaccaacttcccccccgacggccccgtgatgcagaagaagaccatgggctgggaggccagcaccgagcgcctgtaccccgaggacggcgtgctgaagggcgacatcaagatggccctgcgcctgaaggacggcggcagatacctggccgacttcaagaccacctacaaggccaagaagcccgtgcagatgcccggcgcctacaacgtggaccgcaagctggacatcaccagccacaacgaggactacaccgtggtggagcagtacgagcgcagcgagggccgccacagcaccggcggcatggacgagctgtacaagtga

SEQ ID NO: 2 - DNA, 3102 bp, synthetic construct
  source 1..3102: mol_type = other DNA
atggaaacagaggctattgatggctatataacgtgtgacaatgagctttcacccgaaagggagcactccaatatggcaattgacctcacctcaagcacacccaatggacagcatgcctcaccaagtcacatgacaagcacaaattcagtaaagctagaaatgcagagtgatgaagagtgtgacaggaaacccctgagccgtgaagatgagatcaggggccatgatgagggtagcagcctagaagaacccctaattgagagcagcgaggtggctgacaacaggaaagtccaggagcttcaaggcgagggaggaatccggcttccgaatggtaaactgaaatgtgacgtctgtggcatggtttgcattgggcccaatgtgcttatggtacataaaaggagtcacactggtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctgtgggtaaacctcacaagtgcaactactgtggacgaagctacaagcagcgcagttcactggaggagcacaaggaacgctgccacaactatctccagaatgtcagcatggaggctgctgggcaggtcatgagtcaccatgtacctcctatggaagattgtaaggaacaagagcctattatggacaacaatatttctctggtgccttttgagagacctgctgtcatagagaagctcacggggaatatgggaaaacgtaaaagctccactccacaaaagtttgtgggggaaaagctcatgcgattcagctacccagatattcactttgatatgaacttaacatatgagaaggaggctgagctgatgcagtctcatatgatggaccaagccatcaacaatgcaatcacctaccttggagctgaggcccttcaccctctgatgcagcacccgccaagcacaatcgctgaagtggccccagttataagctcagcttattctcaggtctatcatccaaataggatagaaagacccattagcagggaaactgctgatagtcatgaaaacaacatggatggccccatctctctcatcagaccaaagagtcgaccccaggaaagagaggcctctcccagcaatagctgcctggattccactgactcagaaagcagccatgatgaccaccagtcctaccaaggacaccctgccttaaatcccaagaggaaacaaagcccagcttacatgaaggaggatgtcaaagctttggatactaccaaggctcctaagggctctctgaaggacatctacaaggtcttcaatggagaaggagaacagattagggccttcaagtgtgagcactgccgagtccttttcctagaccatgtcatgtacaccattcacatgggttgccatggctaccgggacccactggaatgcaacatctgtggctacagaagccaggaccgttatgagttttcatcacacattgttcgaggggagcacacattccacctcgacggtggaggcggttcaggtggcggaggaagcgggggaggtggaagtatggtgtctaaaggagaagaagataatatggcttctctgcctgctacacacgaactgcatattttcggatctattaatggcgtggatttcgatatggtgggacagggaacaggaaaccctaacgatggatacgaagaactgaatctgaagtctaccaaaggagatctgcaattctctccttggatcctggtccctcacattggatatggattccatcaatatctgccttaccctgacggaatgtctcctttccaagctgctatggtcgatggctctggatatcaggtccacagaacaatgcaatttgaagatggcgcctctctgacagtgaattacagatatacatacgaaggctctcatattaaaggcgaagcccaagtgaaaggcacaggctttcctgccgacggccctgtgatgaccaactctctgaccgctgccgattggtgcagatctaagaagacctaccctaatgataaaaccattatctctaccttcaaatggtcttacacaacaggcaatggcaagagatacagatctaccgccagaaccacctatacctttgctaagcctatggctgccaattacctgaaaaatcagcctatgtatgtgttcagaaaaaccgaactgaaacactctaaaaccgaactgaatttcaaagaatggcaaaaggctttcacagatgtgatgggaatggatgaactgtacaagggaagcggagctacaaattttagtcttcttaaacaagccggtgacgtggaggaaaatcccggcccaatggtgagcaagggcgaggccgtgatcaaggagttcatgcgcttcaaggtgcacatggagggcagcatgaacggccacgagttcgagatcgagggcgagggcgagggccgcccctacgagggcacccagaccgccaagctgaaggtgaccaagggcggccccctgcccttctcttgggacatcctgagcccccagttcatgtacggcagccgcgccttcaccaagcaccccgccgacatccccgactactacaagcagagcttccccgagggcttcaagtgggagcgcgtgatgaacttcgaggacggcggcgccgtgaccgtgacccaggacaccagcctggaggacggcaccctgatctacaaggtgaagctgcgcggcaccaacttcccccccgacggccccgtgatgcagaagaagaccatgggctgggaggccagcaccgagcgcctgtaccccgaggacggcgtgctgaagggcgacatcaagatggccctgcgcctgaaggacggcggcagatacctggccgacttcaagaccacctacaaggccaagaagcccgtgcagatgcccggcgcctacaacgtggaccgcaagctggacatcaccagccacaacgaggactacaccgtggtggagcagtacgagcgcagcgagggccgccacagcaccggcggcatggacgagctgtacaagtga

SEQ ID NO: 3 - DNA, 3051 bp, synthetic construct
  source 1..3051: mol_type = other DNA
atggaagatatacaaacaaatgcggaactgaaaagcactcaggagcagtctgtgcccgcagaaagtgcagcggttttgaatgactacagtttaaccaaatctcatgaaatggaaaatgtggacagtggagaaggcccagccaatgaagatgaagacataggagatgattcaatgaaagtgaaagatgaatacagtgaaagagatgagaatgttttaaagtcagaacccatgggaaatgcagaagagcctgaaatcccttacagctattcaagagaatataatgaatatgaaaacattaagttggagagacatgttgtctcattcgatagtagcaggccaaccagtggaaagatgaactgcgatgtgtgtggattatcctgcatcagcttcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctcacggggcatcttaggacacattctgtggagaaaccctacaaatgtgagttttgtggaaggagttacaagcagagaagttcccttgaggagcacaaggagcgctgccgtacatttcttcagagcactgacccaggggacactgcaagtgcggaggcaagacacatcaaagcagagatgggaagtgaaagagctctcgtactggacagattagcaagcaatgtggcaaaacgaaaaagctcaatgcctcagaaattcattggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagctcgacggtggaggcggttcaggtggcggaggaagcgggggaggtggaagtatggtgtctaaaggagaagaagataatatggcttctctgcctgctacacacgaactgcatattttcggatctattaatggcgtggatttcgatatggtgggacagggaacaggaaaccctaacgatggatacgaagaactgaatctgaagtctaccaaaggagatctgcaattctctccttggatcctggtccctcacattggatatggattccatcaatatctgccttaccctgacggaatgtctcctttccaagctgctatggtcgatggctctggatatcaggtccacagaacaatgcaatttgaagatggcgcctctctgacagtgaattacagatatacatacgaaggctctcatattaaaggcgaagcccaagtgaaaggcacaggctttcctgccgacggccctgtgatgaccaactctctgaccgctgccgattggtgcagatctaagaagacctaccctaatgataaaaccattatctctaccttcaaatggtcttacacaacaggcaatggcaagagatacagatctaccgccagaaccacctatacctttgctaagcctatggctgccaattacctgaaaaatcagcctatgtatgtgttcagaaaaaccgaactgaaacactctaaaaccgaactgaatttcaaagaatggcaaaaggctttcacagatgtgatgggaatggatgaactgtacaagggaagcggagctacaaattttagtcttcttaaacaagccggtgacgtggaggaaaatcccggcccaatggtgagcaagggcgaggccgtgatcaaggagttcatgcgcttcaaggtgcacatggagggcagcatgaacggccacgagttcgagatcgagggcgagggcgagggccgcccctacgagggcacccagaccgccaagctgaaggtgaccaagggcggccccctgcccttctcttgggacatcctgagcccccagttcatgtacggcagccgcgccttcaccaagcaccccgccgacatccccgactactacaagcagagcttccccgagggcttcaagtgggagcgcgtgatgaacttcgaggacggcggcgccgtgaccgtgacccaggacaccagcctggaggacggcaccctgatctacaaggtgaagctgcgcggcaccaacttcccccccgacggccccgtgatgcagaagaagaccatgggctgggaggccagcaccgagcgcctgtaccccgaggacggcgtgctgaagggcgacatcaagatggccctgcgcctgaaggacggcggcagatacctggccgacttcaagaccacctacaaggccaagaagcccgtgcagatgcccggcgcctacaacgtggaccgcaagctggacatcaccagccacaacgaggactacaccgtggtggagcagtacgagcgcagcgagggccgccacagcaccggcggcatggacgagctgtacaagtga